(19) United States
(12) Patent Application Publication
(10) Pub. No.: US 2025/0214977 A1
(43) Pub. Date: Jul. 3, 2025

(54) STAT6 INHIBITORS AND USES THEREOF

(71) Applicant: Kymera Therapeutics, Inc., Watertown, MA (US)

(72) Inventors: Huijun Dong, Watertown, MA (US); Bin Yang, Watertown, MA (US); Bruce C. Follows, Watertown, MA (US); Xiao Zhu, Watertown, MA (US); Matthew M. Weiss, Watertown, MA (US); Xiaozhang Zheng, Watertown, MA (US); Xin Huang, Watertown, MA (US); Lijing Su, Watertown, MA (US); Nello Mainolfi, Watertown, MA (US); Thijs Beuming, Cambridge, MA (US); Xue Fei, Watertown, MA (US); Philip Collier, Watertown, MA (US); Yi Zhang, Watertown, MA (US); Christopher L. Carroll, Watertown, MA (US)

(21) Appl. No.: 18/943,357

(22) Filed: Nov. 11, 2024

Related U.S. Application Data
(60) Provisional application No. 63/649,869, filed on May 20, 2024.
(60) Provisional application No. 63/617,310, filed on Jan. 3, 2024.

(51) Int. Cl. C07D 405/14; A61K 31/443; A61K 31/4433; A61K 31/4436; A61K 31/4439; A61K 31/444; A61K 31/4525; A61K 31/454; A61K 31/4545; A61K 31/4709; A61K 31/4725; A61K 31/496; A61K 31/497; A61K 31/498; A61K 31/4985; A61K 31/501; A61K 31/502; A61K 31/5025; A61K 31/506; A61K 31/517; A61K 31/519; A61K 31/52; A61K 31/5377; A61K 31/538; A61K 31/5383; A61K 31/5386; A61K 31/55; A61K 31/551; A61K 31/553; C07D 401/04; C07D 401/14; C07D 409/14; C07D 413/14; C07D 417/14; C07D 471/04; C07D 487/04; C07D 487/08; C07D 487/10; C07D 491/048; C07D 491/107; C07D 491/22; C07D 495/04; C07D 498/04; C07D 498/08; C07D 498/20

(52) U.S. Cl. CPC C07D 405/14; A61K 31/443; A61K 31/4433; A61K 31/4436; A61K 31/4439; A61K 31/444; A61K 31/4525; A61K 31/454; A61K 31/4545; A61K 31/4709; A61K 31/4725; A61K 31/496; A61K 31/497; A61K 31/498; A61K 31/4985; A61K 31/501; A61K 31/502; A61K 31/5025; A61K 31/506; A61K 31/517; A61K 31/519; A61K 31/52; A61K 31/5377; A61K 31/538; A61K 31/5383; A61K 31/5386; A61K 31/55; A61K 31/551; A61K 31/553; C07D 401/04; C07D 401/14; C07D 409/14; C07D 413/14; C07D 417/14; C07D 471/04; C07D 487/04; C07D 487/08; C07D 487/10; C07D 491/048; C07D 491/107; C07D 491/22; C07D 495/04; C07D 498/04; C07D 498/08; C07D 498/20

(57) ABSTRACT

The present invention provides compounds, compositions, and methods of using the same.

### CROSS-REFERENCE TO RELATED APPLICATIONS

This application claims the benefit of priority to U.S. Provisional Application No. 63/617,310, filed Jan. 3, 2024, and U.S. Provisional Application No. 63/649,869, filed May 20, 2024.

### TECHNICAL FIELD OF THE INVENTION

The present invention relates to inhibitor compounds and methods useful for the inhibition of signal transducer and activator of transcription 6 (“STAT6”). The invention also provides pharmaceutically acceptable compositions comprising inhibitor compounds of the present invention and methods of using said compositions in the treatment of various disorders.

### BACKGROUND OF THE INVENTION

Signal transducer and activator of transcription 6 (STAT6 or Interleukin-4-Stat/IL4-STAT) is an undruggable transcription factor belonging to the structurally conserved Signal Transducer and Activator of Transcription (STAT) family of proteins (STAT1 through STAT6). Activation of STAT6, like other STAT proteins, is triggered upon binding of hormones, immunomodulatory cytokines or growth factors to specific receptors on the cell surface. Once activated, the phosphorylation of a C-terminal tyrosine residue occurs, leading to translocation and transmission of signals from the cytosol to the nucleus, resulting in activation of gene expression.

STAT6 is implicated in driving Type 2 immunity, allergies. It may participate in IL-4/IL-13-mediated allergic reaction, and play a vital role in the differentiation of T-helper type 2 (Th2) cells (Hebenstreit et al. “Signaling mechanisms, interaction partners, and target genes of STAT6.” Cytokine & growth factor reviews 17.3 (2006): 173-188; Chapoval et al. “Regulation of the T helper cell type 2 (Th2)/T regulatory cell (Treg) balance by IL-4 and STAT6.” Journal of leukocyte biology 87.6 (2010): 1011-1018). STAT6 is a key node primarily activated in the Janus Kinase (JAK) pathway by inflammatory cytokines, interleukin-4 (IL4) and interleukin-13 (IL13) and their cognate receptors, which are produced by Th2 cells, mast cells and basophils. Human STAT6 mutations have been associated with severe allergies such as asthma and eczema (Goenka and Kaplan. “Transcriptional regulation by STAT6.” Immunologic research 50.1 (2011): 87-96.). There is a need to discover and develop STAT6 drugs, for example to treat allergic/inflammatory diseases (Glosson et al. “Wheezing and itching: The requirement for STAT proteins in allergic inflammation.” Jak-Stat 1.1 (2012): 3-15; Loh et al. “Signal transducer and activator of transcription (STATs) proteins in cancer and inflammation: functions and therapeutic implication.” Frontiers in oncology 9 (2019): 48). Accordingly, there remains a need to find STAT6 inhibitors useful as therapeutic agents.

### SUMMARY OF THE INVENTION

It has now been found that compounds described herein, and pharmaceutically acceptable compositions thereof, are effective as inhibitors of STAT6 protein.

In some embodiments, the present invention provides inhibitor compounds of formula I′:

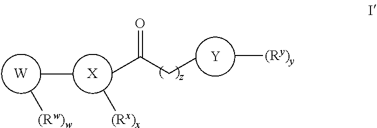

or a pharmaceutically acceptable salt thereof, wherein each variable is as defined and described herein.

Inhibitor compounds described herein, and pharmaceutically acceptable compositions thereof, are useful for treating a variety of diseases, disorders or conditions, associated with regulation of signaling pathways implicating STAT6 protein. Such diseases, disorders, or conditions include those described herein.

In some embodiments, the present invention provides methods of use related to an inhibitor compound of formula I:

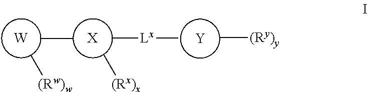

or a pharmaceutically acceptable salt thereof, wherein each variable is as defined and described herein.

Inhibitor compounds described herein are also useful for the study of STAT6 protein in biological and pathological phenomena; the study of intracellular signal transduction pathways occurring in bodily tissues; and the comparative evaluation of new STAT6 inhibitors or other regulators of cell cycling, metastasis, angiogenesis, and immune cell evasion, in vitro or in vivo.

### DETAILED DESCRIPTION OF CERTAIN EMBODIMENTS

### 1. General Description of Certain Embodiments of the Invention

Inhibitor compounds described herein, and compositions thereof, are useful as inhibitors of STAT6 protein which includes phosphorylated or activated STAT6 protein, e.g., pSTAT6. In some embodiments, a provided inhibitor compound inhibits and/or modulates STAT6, pSTAT6, or STAT6 and pSTAT6.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. In some embodiments, a carbocyclic ring may be a 5-12 membered bicyclic, bridged bicyclic, or spirocyclic ring. A carbocyclic ring may include one or more oxo (═O) or thioxo (═S) substituent. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

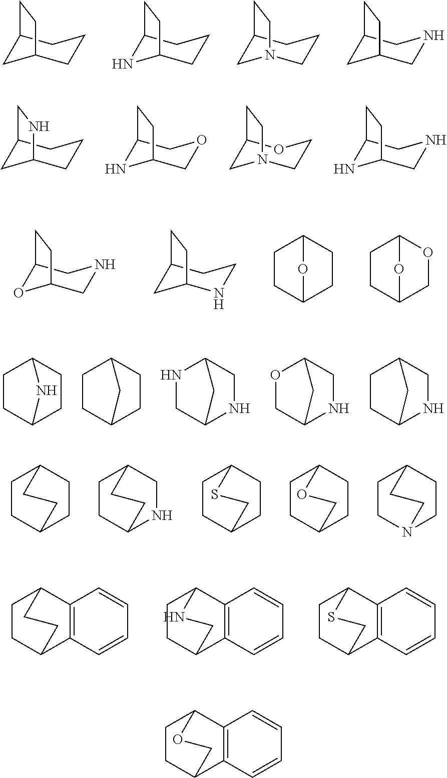

The term “lower alkyl” refers to a C₁₋₄ straight or branched alkyl group. Exemplary lower alkyl groups are methyl, ethyl, propyl, isopropyl, butyl, isobutyl, and tert-butyl.

The term “haloalkyl” refers to a C₁₋₆ straight or branched alkyl group that is substituted with one or more halogen atoms and “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

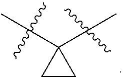 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “arylenyl” refers to bivalent aryl groups (e.g., phenylenyl).

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic, bridged bicyclic, or spirocyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted. The term “heteroarylenyl” refers to bivalent heteroaryl groups (e.g., pyridylenyl).

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. In some embodiments, a heterocyclic ring may be a 5-12 membered bicyclic, bridged bicyclic, or spirocyclic ring. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “substituted” moieties. In general, the term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR^{◯}; —(CH₂)₀₋₄SC(O)R^{◯}; —(CH₂)₀₋₄C(O)NR^{◯}₂; —C(S)NR^{◯}₂; —C(S)SR^{◯}; —SC(S)SR^{◯}, —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —(CH₂)₀₋₄P(O)₂R^{◯}; —(CH₂)₀₋₄P(O)R^{◯}₂; —(CH₂)_{0- 4}OP(O)R^{◯}₂; —(CH₂)₀₋₄OP(O)(OR^{◯})₂; SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}3, —OSiR^{●}3, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃₀—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}2, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₃ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. In some embodiments, the provided compounds are purified in salt form for convenience and/or ease of purification, e.g., using an acidic or basic mobile phase during chromatography. Salts forms of the provided compounds formed during chromatographic purification are contemplated herein (e.g., diammonium salts) and are readily apparent to those having skill in the art.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention

As used herein, the term “provided compound” refers to any genus, subgenus, and/or species set forth herein.

The terms “measurable affinity” and “measurably inhibit,” as used herein, means a measurable change in STAT6 protein activity between a sample comprising a compound of the present invention, or composition thereof, and STAT6 protein, and an equivalent sample comprising STAT6 protein, in the absence of said compound, or composition thereof.

As used herein, the term “reference” describes a standard or control relative to which a comparison is performed. In some embodiments, a “reference” sample or subject is one that is sufficiently similar to a particular sample or subject of interest to permit a relevant comparison. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

As used herein, an “inhibitor compound” is a compound that binds a protein of interest. In some embodiments, an inhibitor compound binds a protein of interest and decreases its activity. In some embodiments, binding of an inhibitor compound to a protein of interest does not result in degradation of the protein of interest. In another embodiment, an inhibitor compound does not bind an E3 ligase in a manner that results in degradation of the protein of interest. In some embodiments, an inhibitor compound binds STAT6. In some embodiments, an inhibitor compound binds STAT6 and decreases its activity. In some embodiments, binding of an inhibitor compound to STAT6 does not result in degradation of STAT6. In another embodiment, an inhibitor compound does not bind an E3 ligase in a manner that results in degradation of the STAT6. In some embodiments, an inhibitor compound binds a protein of interest (e.g., STAT6) but does not bind an E3 ligase. In some embodiments, an E3 ligase is selected from a cereblon E3 ubiquitin ligase, a VHL E3 ubiquitin ligase, a DCAF E3 ubiquitin ligase, (e.g., a DCAF1 E3 ubiquitin ligase, a DCAF15 E3 ubiquitin ligase, or a DCAF16 E3 ubiquitin ligase), an IAP E3 ubiquitin ligase, an MDM2 E3 ligase, or a DC2 E3 ubiquitin ligase. It will be understood that, throughout this disclosure, reference to a “compound” or a “provided compound” refers to an inhibitor compound as defined above.

In some embodiments, an E3 ligase is a cereblon E3 ubiquitin ligase. In some embodiments, the inhibitor compound does not comprise a structure of formula I-aa′:

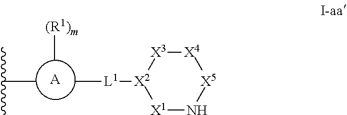

or a pharmaceutically acceptable salt thereof, wherein:
X¹ and X⁵ are independently a covalent bond, —CR₂—, —SO₂—, —S(O)—, —P(O)R—, —P(O)OR—, —P(O)N(R)₂—, —C(O)—, —C(S)—, or

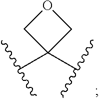 [SMILES: *C1(*)COC1]

X² is N, C—R^{B}, Si—R, or P=O;
X³ and X⁴ are independently a covalent bond, —CR₂—, —CF₂—, —O—, —S—, or X³—X⁴ is —CR═CR—;
each R¹ is independently —H, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R; or:
   two R¹ groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur;
each R^{B} is independently, hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic;
L¹ is a covalent bond or a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂;
Ring A is phenylenyl, naphthalenyl, pyridinylenyl, a 4-7 membered saturated or partially unsaturated carbocyclylenyl, a 4-7 membered saturated or partially unsaturated heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, an 8-10 membered bicyclic heteroarylenyl having 1-5 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same or adjacent atoms or R^{B} and an R group are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur.

In some embodiments, the inhibitor compound does not comprise

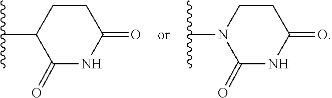 [SMILES: *C1CCC(=O)NC1=O ; *N1CCC(=O)NC1=O]

In some embodiments, an E3 ligase is a VHL E3 ubiquitin ligase. In some embodiments, the inhibitor compound does not comprise a structure of formula I-ccc-3′:

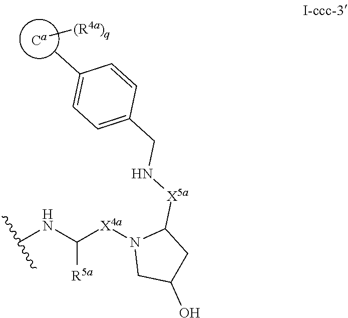

or a pharmaceutically acceptable salt thereof, wherein:
each of X^{4a} and X^{5a} is independently a bivalent moiety selected from —CH₂—, —C(O)—, —C(S)—, or

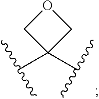 [SMILES: *C1(*)COC1]

each R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R; or:
R^{a} is hydrogen or C₁₋₆ aliphatic;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, a 5-6 membered heteroaryl ring having 1-4 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur;
Ring C^{a} is a selected from 6-membered aryl containing 0-2 nitrogen atoms or a 5-membered heteroaryl with 1-3 heteroatoms independently selected from nitrogen, oxygen or sulfur;
q is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, the inhibitor compound does not comprise

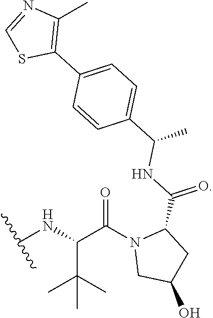 [SMILES: *N[C@H](C(=O)N1C[C@H](O)C[C@H]1C(=O)N[C@@H](C)c1ccc(-c2scnc2C)cc1)C(C)(C)C]

In some embodiments, an E3 ligase is an MDM2 E3 ubiquitin ligase. In some embodiments, the inhibitor compound does not comprise a structure of I-aaa-1, I-aaa-2, I-aaa-3, I-aaa-4, I-aaa-5, I-aaa-6, I-aaa-7, I-aaa-8, I-aaa-9, I-aaa-10, I-aaa-11, I-aaa-12, I-aaa-13, I-aaa-14, I-aaa-15, I-aaa-16, I-aaa-17, or I-aaa-18 respectively:

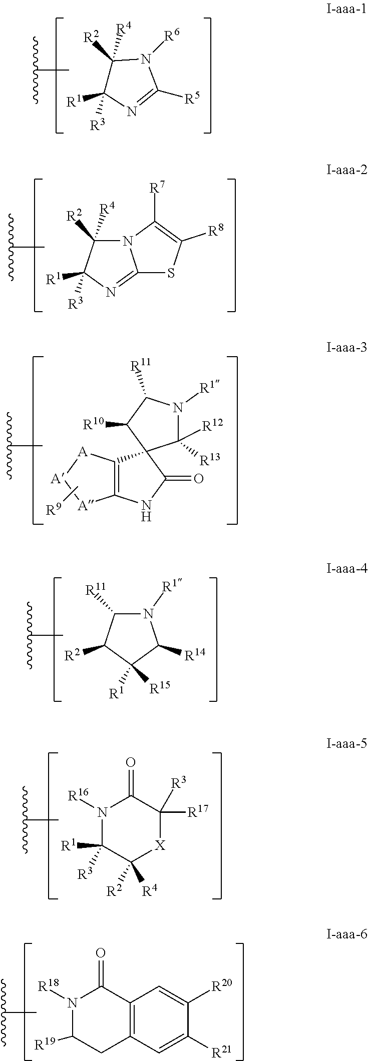

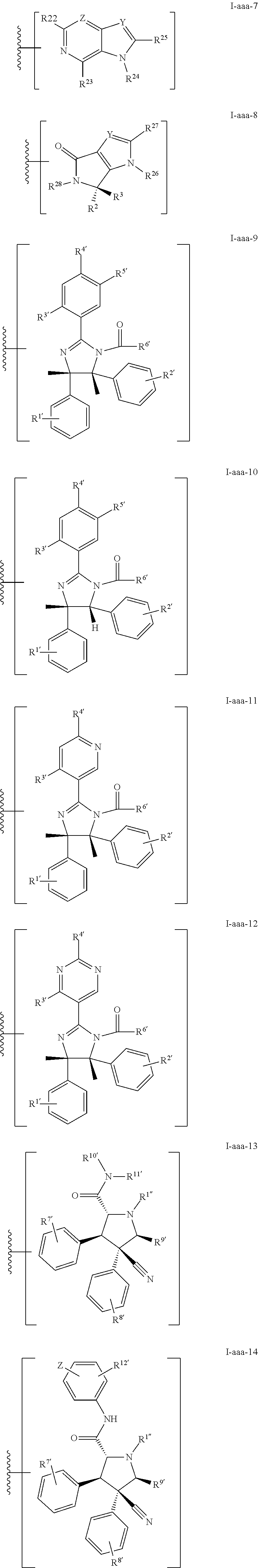

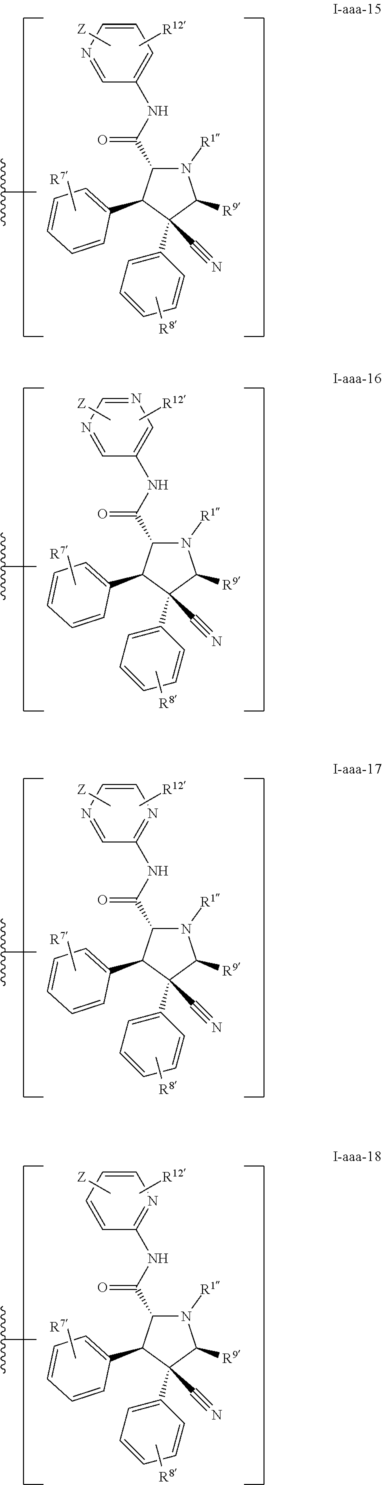

or a pharmaceutically acceptable salt thereof, wherein:
X is selected from —CR₂—, —O—, —S—, —S(O)—, —S(O)₂—, and —NR—;
each R is independently hydrogen or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form a 4-7 membered saturated, partially unsaturated, or heteroaryl ring having 0-3 heteroatoms, in addition to the atom from which they are attached, independently selected from nitrogen, oxygen, and sulfur.
Y and Z are independently selected from —CR═ and —N=;
Ring W is fused ring selected from benzo and a 5-6 membered heteroaryl with 1-4 heteroatoms independently selected from nitrogen, oxygen, and sulfur;
R¹ and R² are independently an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R³ and R⁴ are independently selected from hydrogen and C₁₋₆ alkyl;
R⁵ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R⁶ is selected from hydrogen, —C(O)R, —C(O)OR, and —C(O)NR₂;
R⁷ is selected from hydrogen and R^{A};
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R⁸ is selected from —C(O)R and R^{A};
R⁹ is a mono-, bis-, or tri-substituent on Ring W, wherein each of the substituents are independently selected from halogen and an optionally substituted C₁₋₆ aliphatic;
R¹⁰ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R¹¹ is —C(O)OR or —C(O)NR₂;
R¹² and R¹³ are independently selected from hydrogen and R^{A}, or:
   R¹² and R¹³ are optionally taken together with their intervening atoms to form an optionally substituted 3-8 membered saturated, partially unsaturated, carbocyclic or heterocyclic ring having 1-3 heteroatoms independently selected from nitrogen, oxygen, and sulfur;
R¹⁴ is R^{A};
R¹⁵ is —CN;
R¹⁶ is selected from R^{A}, —OR, —(CR₂)₀₋₆—C(O)R, —(CR₂)₀₋₆—C(O)OR, —(CR₂)₀₋₆—C(O)NR₂, —(CR₂)₀₋₆—S(O)₂R, —(CR₂)₀₋₆—N(R)S(O)₂R, —(CR₂)₀₋₆—S(O)₂NR₂;
R¹⁷ is selected from —(CR₂)₀₋₆—C(O)NR₂;
R¹⁸ and R¹⁹ are independently selected from hydrogen and R^{A};
R²⁰ and R²¹ are independently selected from hydrogen, R^{A}, halogen, and —OR, or:
   R²⁰ and R²¹ are optionally taken together with their intervening atoms to form a fused 5-7 membered partially unsaturated carbocyclic or heterocyclic ring having 1-3 heteroatoms independently selected from nitrogen, oxygen, and sulfur, or a fused 5-6 membered heteroaryl ring having 1-3 heteroatoms independently selected from nitrogen, oxygen, and sulfur;
R²², R²³, R²⁵, and R²⁷ are independently selected from hydrogen, R^{A}, halogen, —C(O)R, —C(O)OR, —C(O)NR₂, —NR₂, —OR, —S(O)R, —S(O)₂R, —S(O)₂NR₂;
R²⁴, R²⁶, and R²⁸ are independently selected from hydrogen, R^{A}, —C(O)R, —C(O)OR, —C(O)NR₂, —S(O)R, —S(O)₂R, and —S(O)₂NR₂;
R^{1′} and R^{2′} are independently selected from halogen, —C≡CR, —CN, —CF₃, and —NO₂;
R^{3′} is —OR;
R^{4′}, R^{5′}, R^{6′} are independently selected from hydrogen, halogen, R^{A}, —CN, —CF₃, —NR₂, —OR, —SR, and —S(O)₂R;
R^{7′} is a mono-, bis-, or tri-substituent, wherein each of the substituents are independently selected from halogen;
R^{8′} is a mono-, bis-, or tri-substituent, wherein each of the substituents are independently selected from hydrogen, halogen, R^{A}, —CN, —C≡CR, —NO₂, and —OR;
R^{9′} is R^{A};
Z¹ is selected from hydrogen, halogen, and —OR;
R^{10′} and R^{11′} are independently selected from hydrogen and R^{A};
R^{12′} is selected from —C(O)R, —C(O)OR, —C(O)NR₂, —OR, —S(O)₂R, —S(O)₂NR₂, and —S(O)R; and
R^{1″} is selected from hydrogen and R^{A}.

In some embodiments, an E3 ligase is a DCAF E3 ubiquitin ligase (e.g., DCAF1 E3 ubiquitin ligase). In some embodiments, the inhibitor compound does not comprise a structure of formula I-b-a or I-b-b:

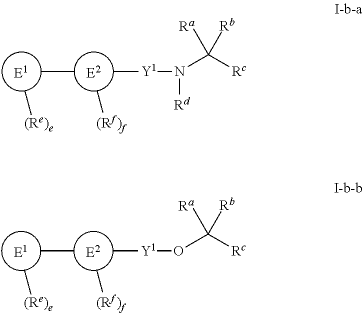

or a pharmaceutically acceptable salt thereof, wherein:
Ring E¹ is phenyl, naphthyl, a 4-9 membered partially unsaturated monocyclic, bicyclic, or bridged bicyclic carbocyclyl or heterocyclyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur;
Ring F¹ is a 5-membered monocyclic heteroarylenyl with 1-3 heteroatoms independently selected from nitrogen, oxygen and sulfur.
Y¹ is a C₁₋₃ hydrocarbon chain wherein each methylene is optionally replaced with —CR₂—, —CR(OR)—, —C(O)—, —C(NR)—, —C(NOR)—, —S(O)—, or —S(O)₂—; or —C(OR)═ in formula I-b-a where R^{d} is absent;
R^{a} is hydrogen, an optionally substituted C₁₋₆ aliphatic, or

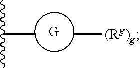

Ring G is phenyl, a 5-7 membered saturated or partially unsaturated carbocyclyl heterocyclyl with 1-3 heteroatoms independently selected from nitrogen, oxygen, and sulfur, or a 5-6 membered heteroaryl with 1-4 heteroatoms independently selected from nitrogen, oxygen and sulfur;
R^{b} is hydrogen, an optionally substituted C₁₋₆ aliphatic, phenyl, or a 5-6 membered heteroaryl with 1-4 heteroatoms independently selected from nitrogen, oxygen and sulfur, or:
   R^{a} and R^{b} are taken together with their intervening atoms to form an optionally substituted 9-10 membered saturated or partially unsaturated bicyclic ring having 0-3 heteroatoms independently selected from nitrogen, oxygen, and sulfur, or:
   when Y¹ is —C(NR)—, R^{b} is taken together with R of —C(NR)— with their intervening atoms to form a 5-7 membered partially unsaturated heterocyclyl with 0-1 heteroatoms, in addition to the 2 heteroatoms within the heterocyclyl, independently selected from nitrogen, oxygen, and sulfur;
R^{c} is —CO₂R, —CONR₂, —CR₂CF₂R, —CR₂CONR₂, —CR₂C(O)R, —CR₂CO₂R, —CR₂NR₂, —CR₂OR, —CR₂SO₂NR₂, —CR₂S(O)R, —CR₂SO₂R, —CR₂S(O)(NR)R, —CR₂CN, —CR₂CR₂NR₂, —CR₂CR₂OR, —CR₂CR═NOR, —CR₂CR(OR)CR₂OR, or an optionally substituted group selected from phenyl; a 4-7 membered saturated or partially unsaturated carbocyclyl or heterocyclyl with 1-3 heteroatoms independently selected from nitrogen, oxygen, and sulfur; a 5-9 membered monocyclic or bicyclic heteroaryl with 1-4 heteroatoms independently selected from nitrogen, oxygen and sulfur; or:
   —(CR₂)₁₋₂—X^{a}, wherein X^{a} is halogen or an optionally substituted ring selected from phenyl; a 4-7 membered saturated or partially unsaturated carbocyclyl or heterocyclyl with 1-3 heteroatoms independently selected from nitrogen, oxygen, and sulfur; and a 5-9 membered monocyclic or bicyclic heteroaryl with 1-4 heteroatoms independently selected from nitrogen, oxygen and sulfur; or:
   R^{b} and R^{c} are taken together with their intervening atoms to form an optionally substituted 4-6 membered saturated or partially unsaturated carbocyclyl or heterocyclyl with 1-3 heteroatoms independently selected from nitrogen, oxygen, and sulfur; or:
   R^{a} is absent and R^{b} and R^{c} are taken together with their intervening atoms to form an optionally substituted phenyl; or:
   when Y¹ is —C(OR)=, R^{c} is taken together with R of —C(OR)═ with their intervening atoms to form a 5-7 membered partially unsaturated heterocyclyl with 0-1 heteroatoms, in addition to the 2 heteroatoms within the heterocyclyl, independently selected from nitrogen, oxygen, and sulfur;
R^{d} is hydrogen or an optionally substituted C₁₋₆ aliphatic, or:
   when R^{c} is —CR₂CONR₂, R^{d} is taken together with a single R of —CR₂CONR₂ with their intervening atoms to form a 5-7 membered saturated or partially unsaturated heterocyclyl with 0-3 heteroatoms, in addition to the nitrogen atom to which R^{d} is attached, independently selected from nitrogen, oxygen, and sulfur;
R^{e}, R^{f}, and R^{g} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —C(NOR)R, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, naphthyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form an optionally substituted 3-7 membered saturated or partially unsaturated ring having 0-3 heteroatoms, in addition to the atom to which they are attached, independently selected from nitrogen, oxygen, and sulfur; and
each of e, f, and g are independently 0, 1, 2, 3, or 4.

In some embodiments, the inhibitor compound does not comprise a structure of formula I-b-c:

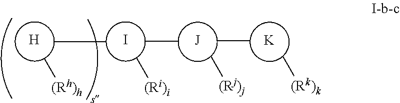

or a pharmaceutically acceptable salt thereof, wherein:
Ring H is a 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
Ring I is phenylenyl or a 5-10 membered monocyclic or bicyclic heteroarylenyl with 1-4 heteroatoms independently selected from nitrogen, oxygen and sulfur;
Ring J is a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
Ring K is phenyl, naphthyl, or a 5-13 membered monocyclic, bicyclic, or tricyclic heteroarylenyl with 1-5 heteroatoms independently selected from nitrogen, oxygen and sulfur;
R^{h}, Rⁱ, R^{j}, and R^{k} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂, or:
   an Rⁱ group on Ring I and an R^{j} group or Ring J are optionally taken together with their intervening atoms to form a 5-8 membered saturated, partially unsaturated, or aromatic ring having 0-3 heteroatoms independently selected from nitrogen, oxygen, and sulfur, or:
each of X¹ and X² are independently a covalent bond, spiro-fusion between the two rings that X¹ or X² connect, or a bivalent, saturated or unsaturated, straight or branched C₁₋₆ hydrocarbon chain, wherein 0-4 methylene units of X¹ and X² are independently replaced by —CR₂—, —CR(OR)—, —CRF—, —CF₂—, —C(NR)—, —C(O)—, —O—, —N(R)—, —S—, —S(O)—, or —S(O)₂—;
s″ is 0 or 1; and
each of w, x, y, and z are independently 0, 1, 2, 3, or 4.

In some embodiments, an E3 ligase is a DC2 E3 ubiquitin ligase. In some embodiments, the inhibitor compound does not comprise a structure of formula I-c-a-1 or I-c-a-2:

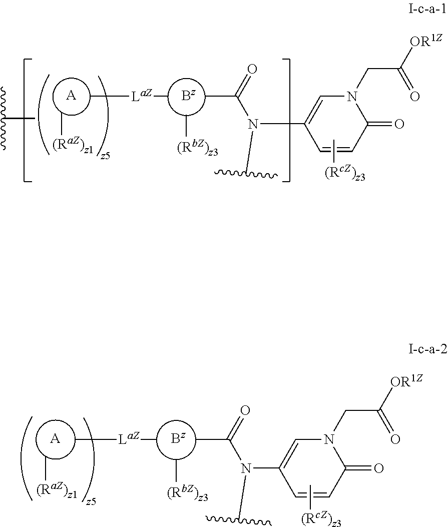

or a pharmaceutically acceptable salt thereof, wherein:
SBM and L are as defined and described above and herein;
R^{1Z} is hydrogen or optionally substituted C₁₋₆ aliphatic;
each R^{aZ}, R^{Z}, and R^{cZ} are independently hydrogen, R^{AZ}, halogen, —CN, —NO₂, —OR^{Z}—SR^{Z}—NR^{Z}₂, —S(O)₂R^{Z}, —S(O)₂NR^{Z}₂, —S(O)R^{Z}—S(O)(NR^{Z})R^{Z}, —P(O)(OR^{Z})₂, —P(O)(NR^{Z}₂)₂, —CFR^{Z}₂, —CR^{Z}F₂, —CF₃, —CR^{Z}₂(OR^{Z}), —CR^{Z}₂(NR^{Z}₂), —C(O)R^{Z}, —C(O)OR^{Z}, or —C(O)NR^{Z}₂;
each R^{AZ} is independently an optionally substituted group selected from C₁₋₁₀ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R^{Z} is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R^{Z} groups on the same atom are optionally taken together with their intervening atom to form an optionally substituted 4-11 membered saturated or partially unsaturated carbocyclic or
heterocyclic monocyclic, bicyclic, bridged bicyclic, spirocyclic, or heteroaryl ring having 0-3 heteroatoms, in addition to the atom to which they are attached, independently selected from nitrogen, oxygen, and sulfur;
each Ring A^{Z} is independently a bivalent ring selected from phenylenyl, naphthylenyl, a 4-10 membered saturated or partially unsaturated monocyclic or bicyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
each Ring B^{Z} is independently a bivalent ring selected from phenylenyl, a 3-10 membered saturated or partially unsaturated monocyclic, bicyclic, bridged bicyclic, or spirocyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
L^{aZ} is absent, a covalent bond, or a C₁₋₃ bivalent straight or branched saturated or unsaturated hydrocarbon chain, wherein 1-3 methylene units of the chain are independently and optionally replaced with —O—, —C(O)—, —C(S)—, —C(R^{Z})₂—, —CH(R^{Z})—, —CF(R^{Z})—, —C(F)₂, —N(R^{Z})—, —S—, —S(O)₂— or —CR^{Z}=CR^{Z}—;
z1, z2, and z3 are each independently 0, 1, 2, 3 or 4;
each of z4 and z5 is independently 0 or 1.

In some embodiments, an inhibitor compound binds to and/or inhibits STAT6 protein with measurable affinity. In certain embodiments, an inhibitor compound has an IC₅₀ and/or binding constant of less than about 50 μM, less than about 1 μM, less than about 500 nM, less than about 100 nM, less than about 10 nM, or less than about 1 nM.

3a. Description of Exemplary Embodiments Related to Methods of Use:

In some embodiments, the present invention provides methods of use related to an inhibitor compound of formula I:

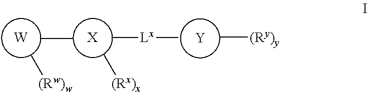

or a pharmaceutically acceptable salt thereof, wherein:
Ring W and its R^{w} substituents is

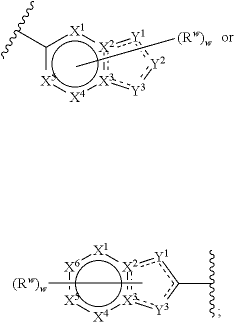

each of X¹, X⁴, X⁵ and X⁶ is independently CH, CR^{w}, or N;
each of X² and X³ is independently C or N,
   wherein at most one of X¹, X², X³, X⁴, X⁵ and X⁶ is N;
each of Y¹, Y², and Y³ is independently CH, CR^{w}, CH₂, CH(R^{w}), C(R^{w})₂, NH, NR^{w}, N, O, or S, wherein at least one of Y² and Y³ is CH, CR^{w}, CH₂, CH(R^{w}), or C(R^{w})₂;
each [symbol] represents a single bond or a double bond;
Ring X and its R^{x} substituents is

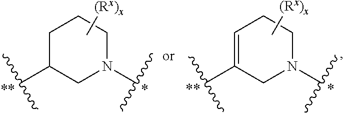

wherein

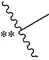

represents a bond to Ring W, and

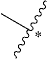

represents a bond to L^{x};
Ring Y is a ring selected from phenyl, naphthyl, 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-10 membered monocyclic or bicyclic heteroaryl with 1-4 heteroatoms independently selected from nitrogen, oxygen and sulfur;
L^{x} is

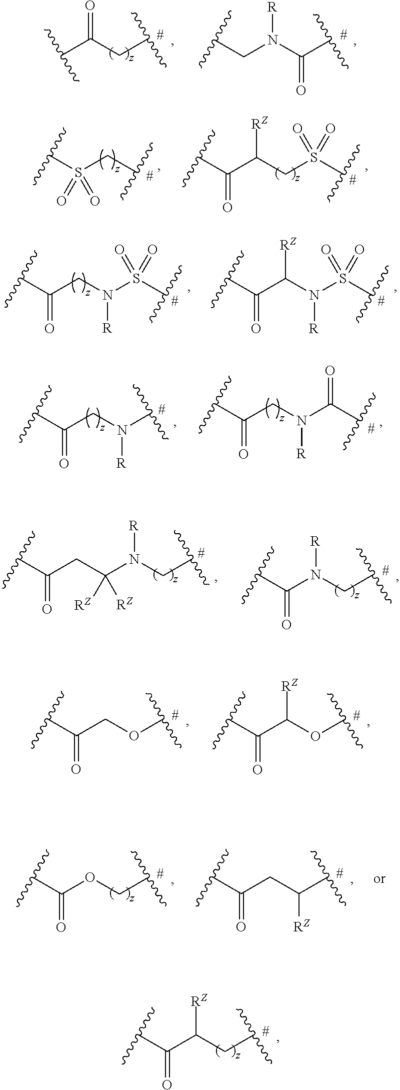

wherein

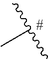

represents a bond to Ring Y;
R^{w} is selected from hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
R^{x} and R^{y} are independently selected from hydrogen, R^{A}, halogen, —CN, —NO₂, —OR, —SR, NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
R^{Z} is R^{A}, —OR, or NR₂;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R^{B} is independently -L^{B}-Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂—, —S(O)(NR)—, or —CR═CR—;
each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, a 5-10 membered monocyclic or bicyclic arylenyl or heteroarylenyl having 1-4 heteroatoms independently selected from nitrogen, oxygen, and sulfur; or a 5-15 membered saturated or partially saturated monocyclic, bicyclic, or tricyclic carbocyclylenyl, heterocyclylenyl, arylenyl, or heteroarylenyl having 1-4 heteroatoms independently selected from nitrogen, oxygen, and sulfur;
each Cy^{B2} is independently an optionally substituted ring selected from phenyl, a 3-10 membered saturated or partially unsaturated monocyclic or bicyclic carbocyclic or heterocyclic ring having 1-2 heteroatoms independently selected from nitrogen, oxygen, and sulfur, a 5-10 membered monocyclic or bicyclic aryl or heteroaryl with 1-4 heteroatoms independently selected from nitrogen, oxygen and sulfur; or a 5-15 membered saturated or partially saturated monocyclic, bicyclic, or tricyclic carbocyclyl, heterocyclyl, aryl, or heteroaryl having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom or adjacent atoms are optionally taken together with their intervening atoms to form a 3-7 membered saturated or partially unsaturated ring having 0-3 heteroatoms, in addition to the atom or adjacent atoms to which they are attached, independently selected from nitrogen, oxygen, and sulfur; and
each of w, x, y, and z are independently 0, 1, 2, 3, or 4.

It will be understood that formula I cannot extend beyond the formulae described within this section (3a) below and herein. It will be understood that all references to “defined and described herein” solely refer to this section (3a) of the application. Additionally, all chemical formulae and embodiments within section 3a can only apply to and be combined with formulae and embodiments from this section (3a).

In some embodiments, the present invention provides methods of use related to an inhibitor compound of formula I, wherein the inhibitor compound is of formula I′:

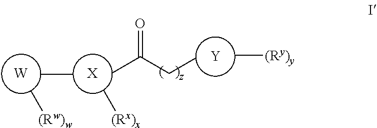

or a pharmaceutically acceptable salt thereof, wherein:
Ring W and its R^{w} substituents is

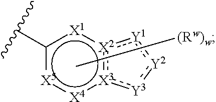

each of X¹, X⁴, and X⁵ is independently CH, CR^{w}, or N;
each of X² and X³ is independently C or N, wherein at most one of X¹, X², X³, X⁴, and X⁵ is N;
each of Y¹, Y², and Y³ is independently CH, CR^{w}, CH₂, CH(R^{w}), C(R^{w})₂, NH, NR^{w}, N, O, or S, wherein at least one of Y² and Y³ is CH, CR^{w}, CH₂, CH(R^{w}), or C(R^{w})₂;
each [symbol] represents a single bond or a double bond;
Ring X and its R^{x} substituents is

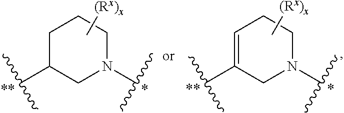

wherein

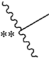

represents a bond to Ring W, and

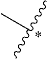

represents a bond to the rest of the molecule;
Ring Y is a ring selected from phenyl, 3-8 membered saturated or partially unsaturated monocyclic carbocyclyl or heterocyclyl with 1-3 heteroatoms independently selected from nitrogen, oxygen, and sulfur, and 5-6 membered monocyclic heteroaryl with 1-4 heteroatoms independently selected from nitrogen, oxygen and sulfur;
R^{w} is selected from hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R^{B} is independently -L^{B}-Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, a 5-10 membered monocyclic or bicyclic arylenyl or heteroarylenyl having 1-4 heteroatoms independently selected from nitrogen, oxygen, and sulfur; or a 5-15 membered saturated or partially saturated monocyclic, bicyclic, or tricyclic carbocyclylenyl, heterocyclylenyl, arylenyl, or heteroarylenyl having 1-4 heteroatoms independently selected from nitrogen, oxygen, and sulfur;
each Cy^{B2} is independently an optionally substituted ring selected from phenyl, a 3-10 membered saturated or partially unsaturated monocyclic or bicyclic carbocyclic or heterocyclic ring having 1-2 heteroatoms independently selected from nitrogen, oxygen, and sulfur, a 5-10 membered monocyclic or bicyclic aryl or heteroaryl with 1-4 heteroatoms independently selected from nitrogen, oxygen and sulfur; or a 5-15 membered saturated or partially saturated monocyclic, bicyclic, or tricyclic carbocyclyl, heterocyclyl, aryl, or heteroaryl having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom or adjacent atoms are optionally taken together with their intervening atoms to form a 3-7 membered saturated or partially unsaturated ring having 0-3 heteroatoms, in addition to the atom or adjacent atoms to which they are attached, independently selected from nitrogen, oxygen, and sulfur; and
each of w, x, y, and z are independently 0, 1, 2, 3, or 4.

In some embodiments, the present invention provides methods of use related to an inhibitor compound of formula I, wherein the inhibitor compound is of formula II:

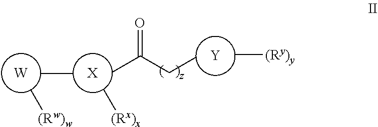

or a pharmaceutically acceptable salt thereof, wherein:
Ring W and its R^{w} substituents is

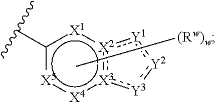

each of X¹, X⁴, and X⁵ is independently CH, CR^{w}, or N;
each of X² and X³ is independently C or N,
   wherein at most one of X¹, X², X³, X⁴, and X⁵ is N;
each of Y¹, Y², and Y³ is independently CH, CR^{w}, CH₂, CH(R^{w}), C(R^{w})₂, NH, NR^{w}, N, O, or S,
   wherein at least one of Y² and Y³ is CH, CR^{w}, CH₂, CH(R^{w}), or C(R^{w})₂;
each [symbol] represents a single bond or a double bond;
Ring X and its R^{x} substituents is

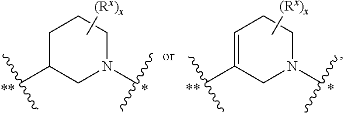

wherein

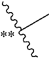

represents a bond to Ring W, and

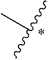

represents a bond to the rest of the molecule;
Ring Y is a ring selected from naphthyl, 5-10 membered saturated or partially unsaturated bicyclic or bridged bicyclic carbocyclyl or heterocyclyl with 1-3 heteroatoms independently selected from nitrogen, oxygen, and sulfur, and 8-10 membered bicyclic heteroaryl with 1-4 heteroatoms independently selected from nitrogen, oxygen and sulfur;
R^{w} is selected from hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R^{B} is independently -L^{B}-Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, a 5-10 membered monocyclic or bicyclic arylenyl or heteroarylenyl having 1-4 heteroatoms independently selected from nitrogen, oxygen, and sulfur; or a 5-15 membered saturated or partially saturated monocyclic, bicyclic, or tricyclic carbocyclylenyl, heterocyclylenyl, arylenyl, or heteroarylenyl having 1-4 heteroatoms independently selected from nitrogen, oxygen, and sulfur;
each Cy^{B2} is independently an optionally substituted ring selected from phenyl, a 3-10 membered saturated or partially unsaturated monocyclic or bicyclic carbocyclic or heterocyclic ring having 1-2 heteroatoms independently selected from nitrogen, oxygen, and sulfur, a 5-10 membered monocyclic or bicyclic aryl or heteroaryl with 1-4 heteroatoms independently selected from nitrogen, oxygen and sulfur; or a 5-15 membered saturated or partially saturated monocyclic, bicyclic, or tricyclic carbocyclyl, heterocyclyl, aryl, or heteroaryl having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom or adjacent atoms are optionally taken together with their intervening atoms to form a 3-7 membered saturated or partially unsaturated ring having 0-3 heteroatoms, in addition to the atom or adjacent atoms to which they are attached, independently selected from nitrogen, oxygen, and sulfur; and
each of w, x, y, and z are independently 0, 1, 2, 3, or 4.

In some embodiments, the present invention provides methods of use related to an inhibitor compound of formula I, wherein the inhibitor compound is of formula III:

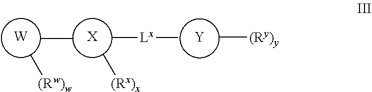

or a pharmaceutically acceptable salt thereof, wherein:
Ring W and its R^{w} substituents is

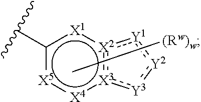

each of X¹, X⁴, and X⁵ is independently CH, CR^{w}, or N;
each of X² and X³ is independently C or N,
   wherein at most one of X¹, X², X³, X⁴, and X⁵ is N;
each of Y¹, Y², and Y³ is independently CH, CR^{w}, CH₂, CH(R^{w}), C(R^{w})₂, NH, NR^{w}, N, O, or S,
   wherein at least one of Y² and Y³ is CH, CR^{w}, CH₂, CH(R^{w}), or C(R^{w})₂;
each [symbol] represents a single bond or a double bond;
Ring X and its R^{x} substituents is

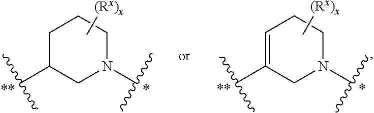

wherein

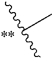

represents a bond to Ring W, and

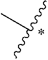

represents a bond to L^{x};
Ring Y is a ring selected from phenyl, 3-8 membered saturated or partially unsaturated monocyclic carbocyclyl or heterocyclyl with 1-3 heteroatoms independently selected from nitrogen, oxygen, and sulfur, 5-6 membered monocyclic heteroaryl with 1-4 heteroatoms independently selected from nitrogen, oxygen and sulfur, naphthyl, 5-10 membered saturated or partially unsaturated bicyclic or bridged bicyclic carbocyclyl or heterocyclyl with 1-3 heteroatoms independently selected from nitrogen, oxygen, and sulfur, and 8-10 membered bicyclic heteroaryl with 1-4 heteroatoms independently selected from nitrogen, oxygen and sulfur;
L^{x} is

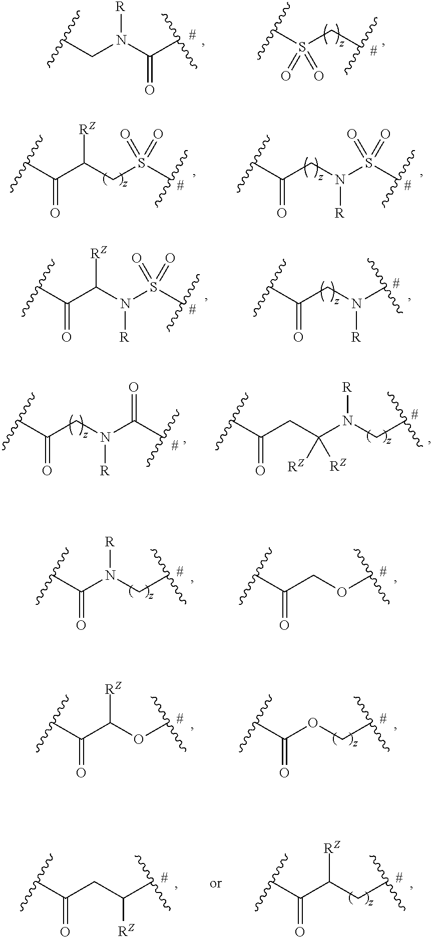

wherein

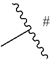

represents a bond to Ring Y;
R^{w} is selected from hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
R^{Z} is R^{A}, —OR, or NR₂;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R^{B} is independently -L^{B}-Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, a 5-10 membered monocyclic or bicyclic arylenyl or heteroarylenyl having 1-4 heteroatoms independently selected from nitrogen, oxygen, and sulfur; or a 5-15 membered saturated or partially saturated monocyclic, bicyclic, or tricyclic carbocyclylenyl, heterocyclylenyl, arylenyl, or heteroarylenyl having 1-4 heteroatoms independently selected from nitrogen, oxygen, and sulfur;
each Cy^{B2} is independently an optionally substituted ring selected from phenyl, a 3-10 membered saturated or partially unsaturated monocyclic or bicyclic carbocyclic or heterocyclic ring having 1-2 heteroatoms independently selected from nitrogen, oxygen, and sulfur, a 5-10 membered monocyclic or bicyclic aryl or heteroaryl with 1-4 heteroatoms independently selected from nitrogen, oxygen and sulfur; or a 5-15 membered saturated or partially saturated monocyclic, bicyclic, or tricyclic carbocyclyl, heterocyclyl, aryl, or heteroaryl having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom or adjacent atoms are optionally taken together with their intervening atoms to form a 3-7 membered saturated or partially unsaturated ring having 0-3 heteroatoms, in addition to the atom or adjacent atoms to which they are attached, independently selected from nitrogen, oxygen, and sulfur; and
each of w, x, y, and z are independently 0, 1, 2, 3, or 4.

In some embodiments, the present invention provides methods of use related to an inhibitor compound of formula I, wherein the inhibitor compound is of formula IV:

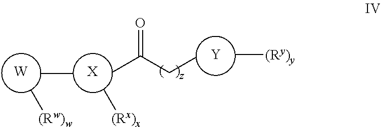

or a pharmaceutically acceptable salt thereof, wherein:
Ring W and its R^{w} substituents is

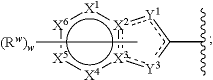

each of X¹, X⁴, X⁵ and X⁶ is independently CH, CR^{w}, or N;
each of X² and X³ is independently C or N, wherein at most one of X¹, X², X³, X⁴, X⁵ and X⁶ is N;
each of Y¹ and Y³ is independently CH, CR^{w}, CH₂, CH(R^{w}), C(R^{w})₂, NH, NR^{w}, N, O, or S;
each [symbol] represents a single bond or a double bond;
Ring X and its R^{x} substituents is

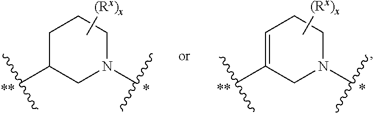

wherein

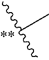

represents a bond to Ring W, and

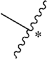

represents a bond to the rest of the molecule;
Ring Y is a ring selected from phenyl, 3-8 membered saturated or partially unsaturated monocyclic carbocyclyl or heterocyclyl with 1-3 heteroatoms independently selected from nitrogen, oxygen, and sulfur, and 5-6 membered monocyclic heteroaryl with 1-4 heteroatoms independently selected from nitrogen, oxygen and sulfur;
R^{w} is selected from hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
R^{Z} is R^{A}, —OR, or NR₂;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R^{B} is independently -L^{B}-Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, a 5-10 membered monocyclic or bicyclic arylenyl or heteroarylenyl having 1-4 heteroatoms independently selected from nitrogen, oxygen, and sulfur; or a 5-15 membered saturated or partially saturated monocyclic, bicyclic, or tricyclic carbocyclylenyl, heterocyclylenyl, arylenyl, or heteroarylenyl having 1-4 heteroatoms independently selected from nitrogen, oxygen, and sulfur;
each Cy^{B2} is independently an optionally substituted ring selected from phenyl, a 3-10 membered saturated or partially unsaturated monocyclic or bicyclic carbocyclic or heterocyclic ring having 1-2 heteroatoms independently selected from nitrogen, oxygen, and sulfur, a 5-10 membered monocyclic or bicyclic aryl or heteroaryl with 1-4 heteroatoms independently selected from nitrogen, oxygen and sulfur; or a 5-15 membered saturated or partially saturated monocyclic, bicyclic, or tricyclic carbocyclyl, heterocyclyl, aryl, or heteroaryl having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom or adjacent atoms are optionally taken together with their intervening atoms to form a 3-7 membered saturated or partially unsaturated ring having 0-3 heteroatoms, in addition to the atom or adjacent atoms to which they are attached, independently selected from nitrogen, oxygen, and sulfur; and
each of w, x, y, and z are independently 0, 1, 2, 3, or 4.

In some embodiments, the present invention provides methods of use related to an inhibitor compound of formula I′, wherein:
(i) when Ring X and its R^{x} substituents is

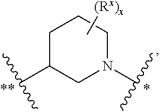

X¹ is N, and Y¹ is NH, NR^{w}, or N, then Y² and Y³ are not NH, NR^{w}, or N;
(ii) when Ring X and its R^{x} substituents is

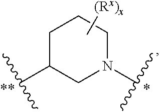

each of X¹, X⁴, and X⁵ is CH or CR^{w}, each of X² and X³ is C, and each of Y² and Y³ is CH, CR^{w}, CH₂, CH(R^{w}), or C(R^{w})₂, then Y¹ is not CH, CR^{w}, CH₂, CH(R^{w}), or C(R^{w})₂;
(iii) when Ring X and its R^{x} substituents is

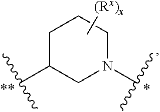

X¹ is N, and Y¹ is S, then Ring Y is not phenyl or 3-8 membered saturated or partially unsaturated monocyclic carbocyclyl or heterocyclyl with 1-3 heteroatoms independently selected from nitrogen, oxygen, and sulfur;
(iv) when Ring X and its R^{x} substituents is

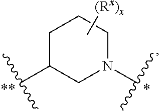

X¹ is N, and Y¹ is NH, NR^{w}, or N, then Y³ is not S;
(v) when Ring X and its R^{x} substituents is

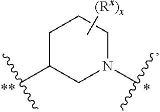

X¹ is N, and Y¹ is O, then Y² is not NH, NR^{w}, or N;
(vi) when Ring X and its R^{x} substituents is

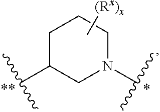

X¹ is N, Y¹ is NH, NR^{w}, or N, and Y² and Y³ are CH, CR^{w}, CH₂, CH(R^{w}), or C(R^{w})₂, then z is not 0 or 1;
(vii) when Ring X and its R^{x} substituents is

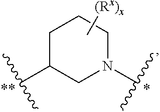

each of X¹, X⁴, and X⁵ is CH or CR^{w}, each of X² and X³ is C, and Y² is CH, CR^{w}, CH₂, CH(R^{w}), or C(R^{w})₂, then only one of Y² and Y³ is O;
(viii) when Ring X and its R^{x} substituents is

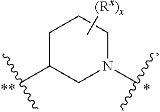

each of X¹, X⁴, and X⁵ is CH or CR^{w}, each of X² and X³ is C, Y¹ is O, NH, NR^{w}, or N, and Y² and Y³ are CH, CR^{w}, CH₂, CH(R^{w}), or C(R^{w})₂, then z is not 0 or 1; and
(ix) when Ring X and its R^{x} substituents is

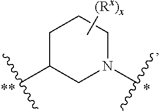

X⁵ is N, and Y¹ is NH, NR^{w}, or N, then Y² is not NH, NR^{w}, or N.

In some embodiments, the inhibitor compound of formula I, I′, II, III, or IV is not a compound of structure A-B-C defined by the combination of the building blocks A, B, and C within the table below:

| A | B | C |
|---|---|---|
| 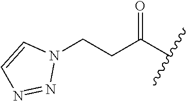 [SMILES: *C(=O)CCn1ccnn1] | 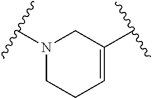 [SMILES: *C1=CCCN(*)C1] | 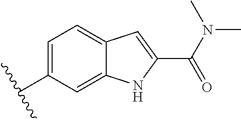 [SMILES: *c1ccc2cc(C(=O)N(C)C)[nH]c2c1] |
| 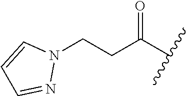 [SMILES: *C(=O)CCn1cccn1] | | 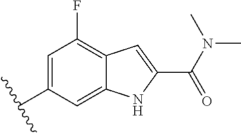 [SMILES: *c1cc(F)c2cc(C(=O)N(C)C)[nH]c2c1] |
| 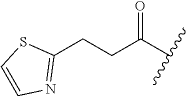 [SMILES: *C(=O)CCc1nccs1] | | 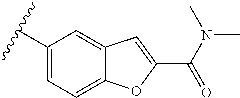 [SMILES: *c1ccc2oc(C(=O)N(C)C)cc2c1] |
| 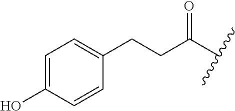 [SMILES: *C(=O)CCc1ccc(O)cc1] | | 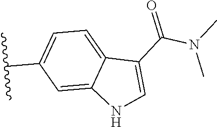 [SMILES: *c1ccc2c(C(=O)N(C)C)c[nH]c2c1] |
| 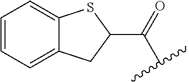 [SMILES: *C(=O)C1Cc2ccccc2S1] | | |

In some embodiments, the inhibitor compound of formula I, I′, II, III, or IV is not any of compounds I-1 through I-1548, or pharmaceutically acceptable salts thereof, described in PCT/US2024/044544. In some embodiments, the inhibitor compound of formula I, I′, II, III, or IV is not a compound of Table 1, or a pharmaceutically acceptable salt thereof, of International Application No. PCT/US2024/044544.

In some embodiments, the inhibitor compound of formula I, I′, II, III, or IV is not a compound selected from Table A.

TABLE A

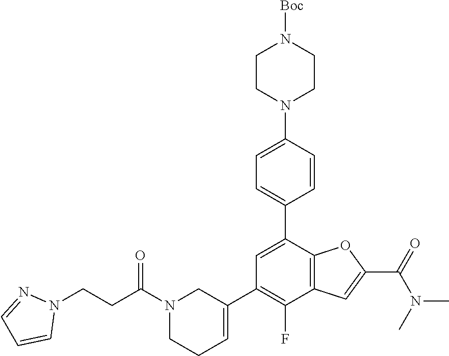 [SMILES: CN(C)C(=O)c1cc2c(F)c(C3=CCCN(C(=O)CCn4cccn4)C3)cc(-c3ccc(N4CCN(C(=O)OC(C)(C)C)CC4)cc3)c2o1]

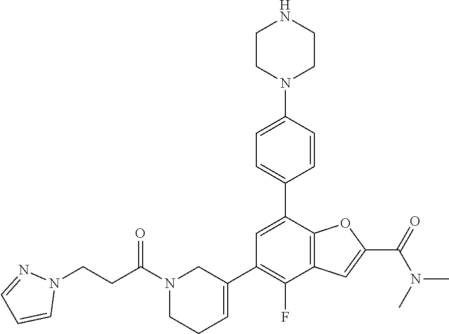 [SMILES: CN(C)C(=O)c1cc2c(F)c(C3=CCCN(C(=O)CCn4cccn4)C3)cc(-c3ccc(N4CCNCC4)cc3)c2o1]

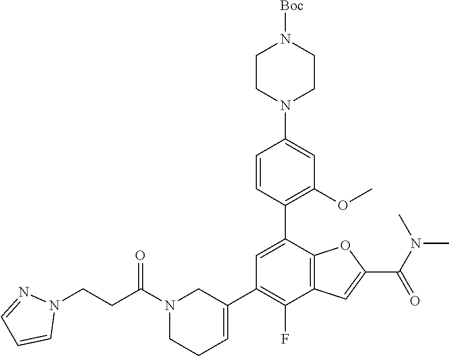 [SMILES: COc1cc(N2CCN(C(=O)OC(C)(C)C)CC2)ccc1-c1cc(C2=CCCN(C(=O)CCn3cccn3)C2)c(F)c2cc(C(=O)N(C)C)oc12]

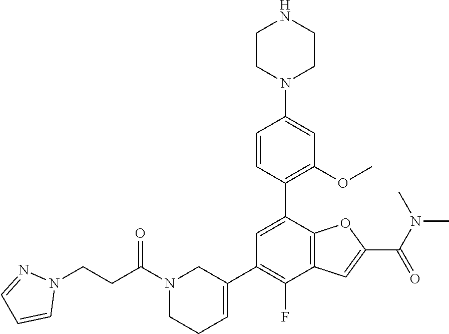 [SMILES: COc1cc(N2CCNCC2)ccc1-c1cc(C2=CCCN(C(=O)CCn3cccn3)C2)c(F)c2cc(C(=O)N(C)C)oc12]

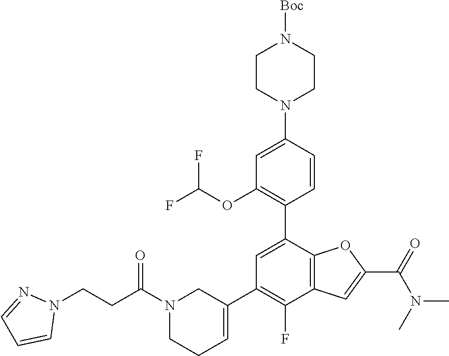 [SMILES: CN(C)C(=O)c1cc2c(F)c(C3=CCCN(C(=O)CCn4cccn4)C3)cc(-c3ccc(N4CCN(C(=O)OC(C)(C)C)CC4)cc3OC(F)F)c2o1]

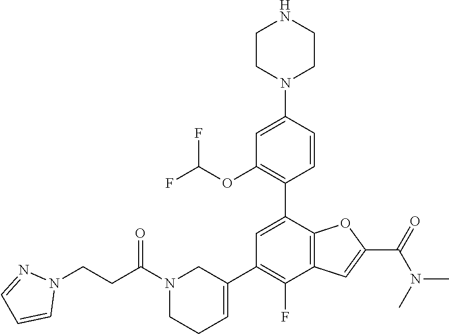 [SMILES: CN(C)C(=O)c1cc2c(F)c(C3=CCCN(C(=O)CCn4cccn4)C3)cc(-c3ccc(N4CCNCC4)cc3OC(F)F)c2o1]

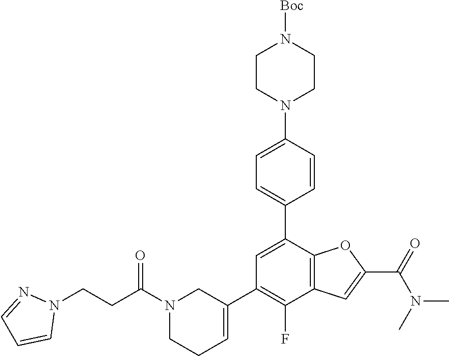 [SMILES: CN(C)C(=O)c1cc2c(F)c(C3=CCCN(C(=O)CCn4cccn4)C3)cc(-c3ccc(N4CCN(C(=O)OC(C)(C)C)CC4)cc3)c2o1]

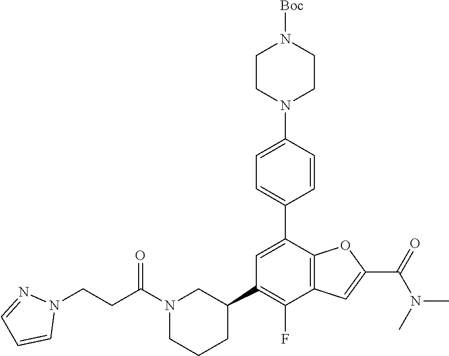 [SMILES: CN(C)C(=O)c1cc2c(F)c([C@H]3CCCN(C(=O)CCn4cccn4)C3)cc(-c3ccc(N4CCN(C(=O)OC(C)(C)C)CC4)cc3)c2o1]

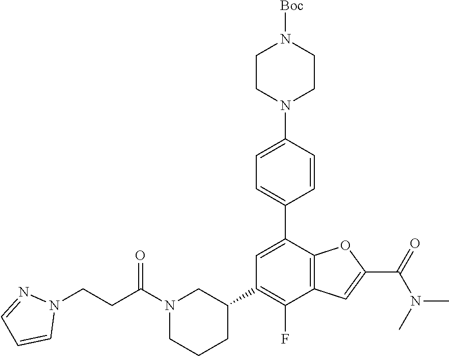 [SMILES: CN(C)C(=O)c1cc2c(F)c([C@@H]3CCCN(C(=O)CCn4cccn4)C3)cc(-c3ccc(N4CCN(C(=O)OC(C)(C)C)CC4)cc3)c2o1]

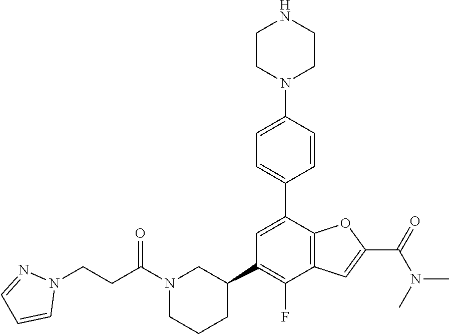 [SMILES: CN(C)C(=O)c1cc2c(F)c([C@H]3CCCN(C(=O)CCn4cccn4)C3)cc(-c3ccc(N4CCNCC4)cc3)c2o1]

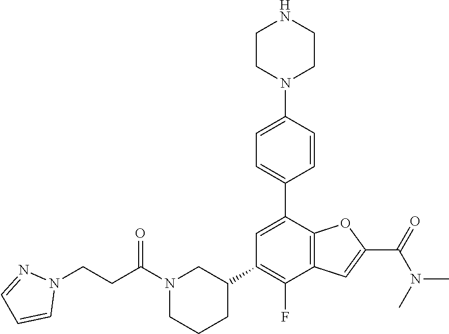

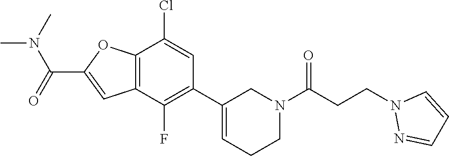 [SMILES: CN(C)C(=O)c1cc2c(F)c(C3=CCCN(C(=O)CCn4cccn4)C3)cc(Cl)c2o1]

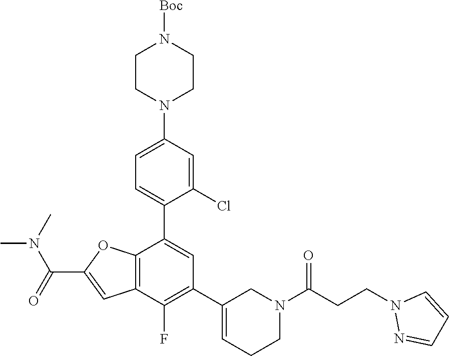 [SMILES: CN(C)C(=O)c1cc2c(F)c(C3=CCCN(C(=O)CCn4cccn4)C3)cc(-c3ccc(N4CCN(C(=O)OC(C)(C)C)CC4)cc3Cl)c2o1]

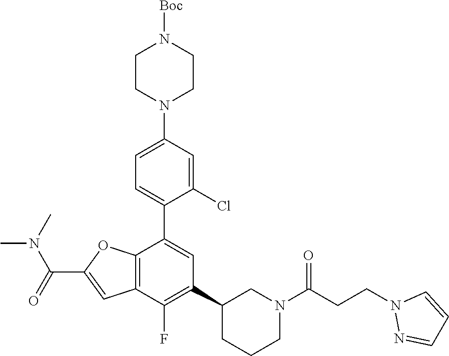 [SMILES: CN(C)C(=O)c1cc2c(F)c([C@@H]3CCCN(C(=O)CCn4cccn4)C3)cc(-c3ccc(N4CCN(C(=O)OC(C)(C)C)CC4)cc3Cl)c2o1]

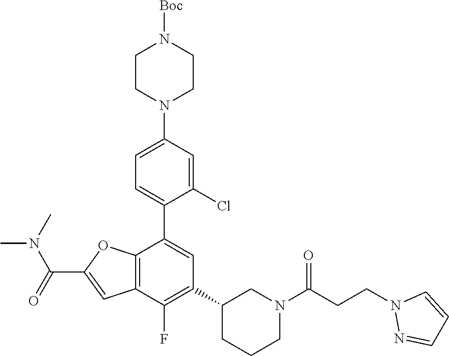 [SMILES: CN(C)C(=O)c1cc2c(F)c([C@H]3CCCN(C(=O)CCn4cccn4)C3)cc(-c3ccc(N4CCN(C(=O)OC(C)(C)C)CC4)cc3Cl)c2o1]

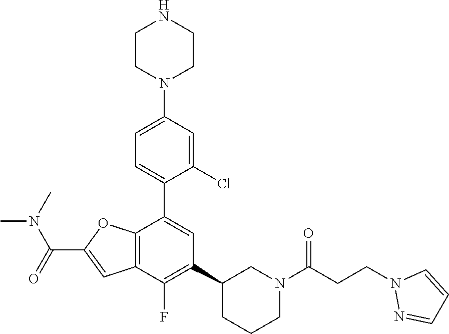 [SMILES: CN(C)C(=O)c1cc2c(F)c([C@@H]3CCCN(C(=O)CCn4cccn4)C3)cc(-c3ccc(N4CCNCC4)cc3Cl)c2o1]

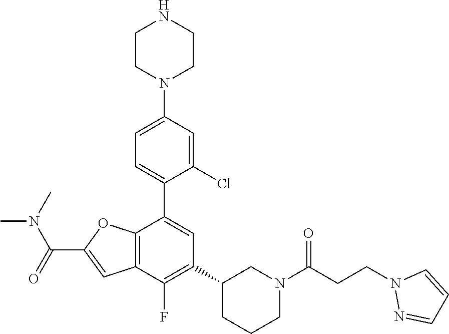 [SMILES: CN(C)C(=O)c1cc2c(F)c([C@H]3CCCN(C(=O)CCn4cccn4)C3)cc(-c3ccc(N4CCNCC4)cc3Cl)c2o1]

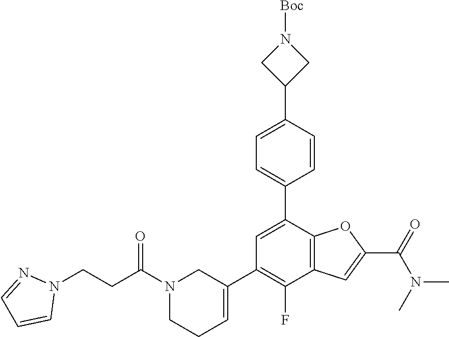 [SMILES: CN(C)C(=O)c1cc2c(F)c(C3=CCCN(C(=O)CCn4cccn4)C3)cc(-c3ccc(C4CN(C(=O)OC(C)(C)C)C4)cc3)c2o1]

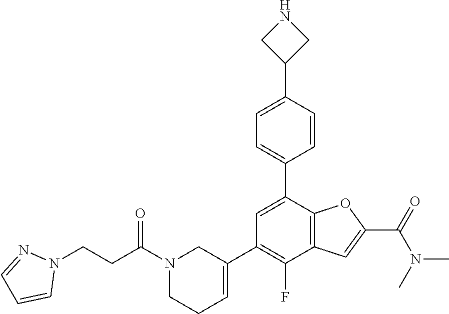 [SMILES: CN(C)C(=O)c1cc2c(F)c(C3=CCCN(C(=O)CCn4cccn4)C3)cc(-c3ccc(C4CNC4)cc3)c2o1]

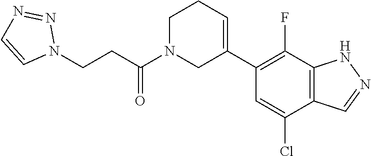 [SMILES: O=C(CCn1ccnn1)N1CCC=C(c2cc(Cl)c3cn[nH]c3c2F)C1]

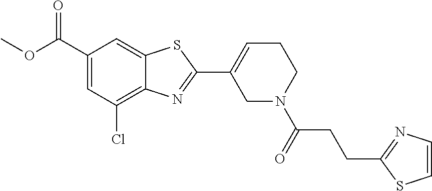 [SMILES: COC(=O)c1cc(Cl)c2nc(C3=CCCN(C(=O)CCc4nccs4)C3)sc2c1]

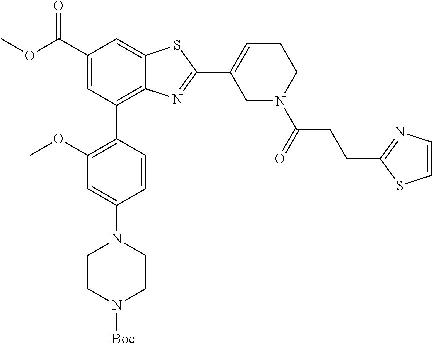 [SMILES: COC(=O)c1cc(-c2ccc(N3CCN(C(=O)OC(C)(C)C)CC3)cc2OC)c2nc(C3=CCCN(C(=O)CCc4nccs4)C3)sc2c1]

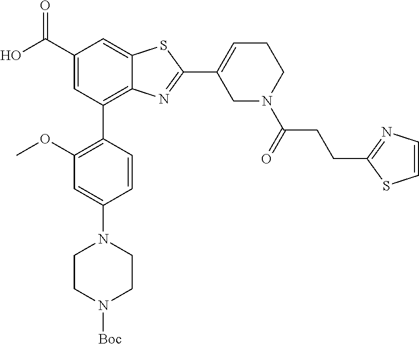 [SMILES: COc1cc(N2CCN(C(=O)OC(C)(C)C)CC2)ccc1-c1cc(C(=O)O)cc2sc(C3=CCCN(C(=O)CCc4nccs4)C3)nc12]

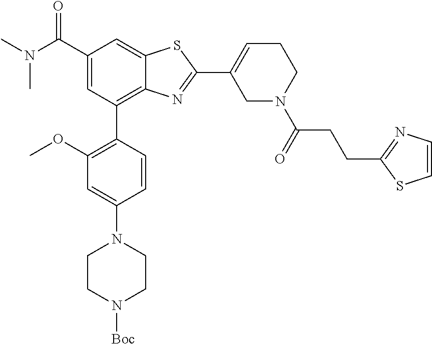 [SMILES: COc1cc(N2CCN(C(=O)OC(C)(C)C)CC2)ccc1-c1cc(C(=O)N(C)C)cc2sc(C3=CCCN(C(=O)CCc4nccs4)C3)nc12]

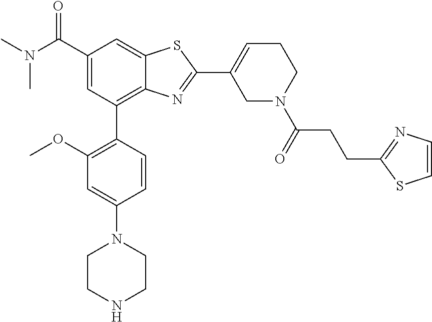 [SMILES: COc1cc(N2CCNCC2)ccc1-c1cc(C(=O)N(C)C)cc2sc(C3=CCCN(C(=O)CCc4nccs4)C3)nc12]

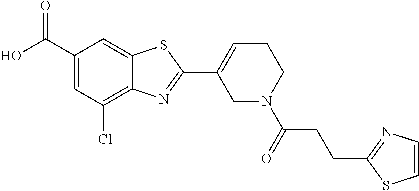 [SMILES: O=C(O)c1cc(Cl)c2nc(C3=CCCN(C(=O)CCc4nccs4)C3)sc2c1]

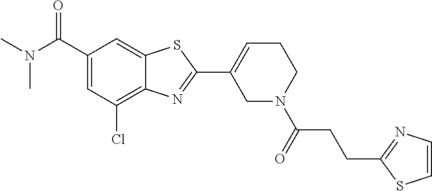 [SMILES: CN(C)C(=O)c1cc(Cl)c2nc(C3=CCCN(C(=O)CCc4nccs4)C3)sc2c1]

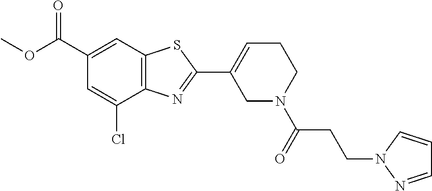 [SMILES: COC(=O)c1cc(Cl)c2nc(C3=CCCN(C(=O)CCn4cccn4)C3)sc2c1]

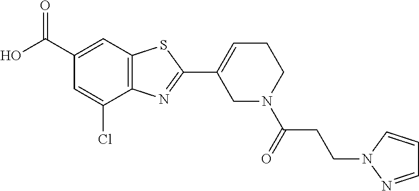 [SMILES: O=C(O)c1cc(Cl)c2nc(C3=CCCN(C(=O)CCn4cccn4)C3)sc2c1]

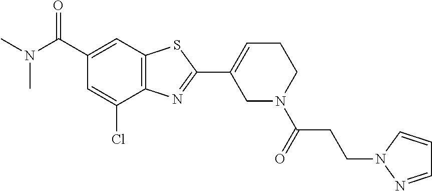 [SMILES: CN(C)C(=O)c1cc(Cl)c2nc(C3=CCCN(C(=O)CCn4cccn4)C3)sc2c1]

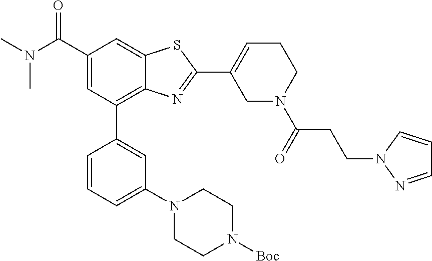 [SMILES: CN(C)C(=O)c1cc(-c2cccc(N3CCN(C(=O)OC(C)(C)C)CC3)c2)c2nc(C3=CCCN(C(=O)CCn4cccn4)C3)sc2c1]

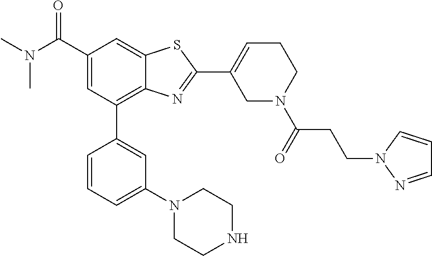 [SMILES: CN(C)C(=O)c1cc(-c2cccc(N3CCNCC3)c2)c2nc(C3=CCCN(C(=O)CCn4cccn4)C3)sc2c1]

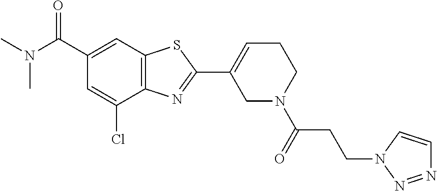 [SMILES: CN(C)C(=O)c1cc(Cl)c2nc(C3=CCCN(C(=O)CCn4ccnn4)C3)sc2c1]

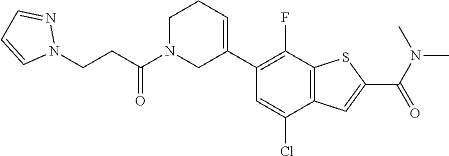 [SMILES: CN(C)C(=O)c1cc2c(Cl)cc(C3=CCCN(C(=O)CCn4cccn4)C3)c(F)c2s1]

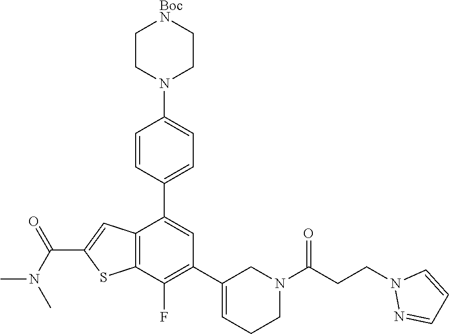 [SMILES: CN(C)C(=O)c1cc2c(-c3ccc(N4CCN(C(=O)OC(C)(C)C)CC4)cc3)cc(C3=CCCN(C(=O)CCn4cccn4)C3)c(F)c2s1]

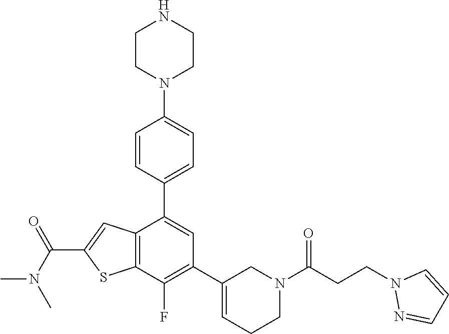 [SMILES: CN(C)C(=O)c1cc2c(-c3ccc(N4CCNCC4)cc3)cc(C3=CCCN(C(=O)CCn4cccn4)C3)c(F)c2s1]

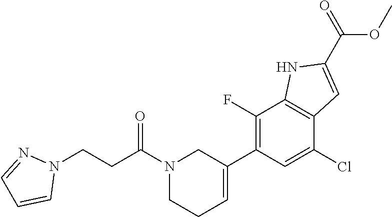 [SMILES: COC(=O)c1cc2c(Cl)cc(C3=CCCN(C(=O)CCn4cccn4)C3)c(F)c2[nH]1]

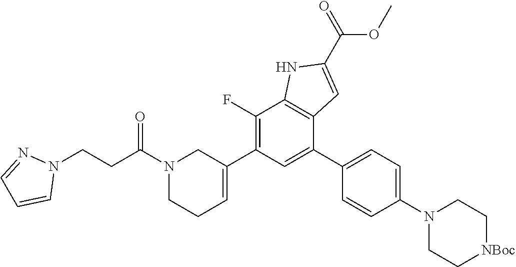 [SMILES: COC(=O)c1cc2c(-c3ccc(N4CCN(C(=O)OC(C)(C)C)CC4)cc3)cc(C3=CCCN(C(=O)CCn4cccn4)C3)c(F)c2[nH]1]

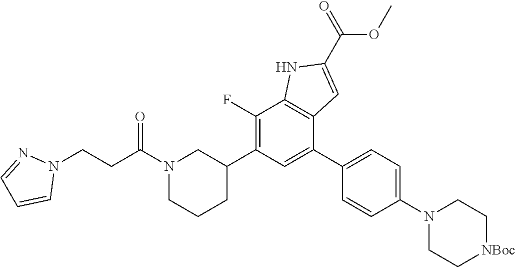 [SMILES: COC(=O)c1cc2c(-c3ccc(N4CCN(C(=O)OC(C)(C)C)CC4)cc3)cc(C3CCCN(C(=O)CCn4cccn4)C3)c(F)c2[nH]1]

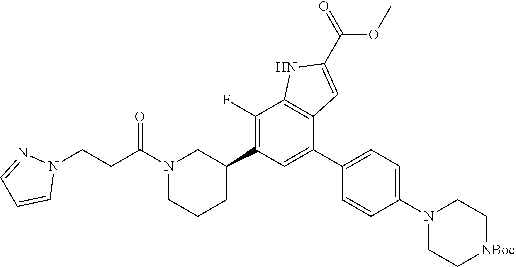 [SMILES: COC(=O)c1cc2c(-c3ccc(N4CCN(C(=O)OC(C)(C)C)CC4)cc3)cc([C@H]3CCCN(C(=O)CCn4cccn4)C3)c(F)c2[nH]1]

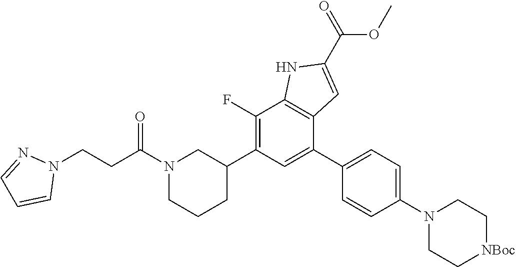 [SMILES: COC(=O)c1cc2c(-c3ccc(N4CCN(C(=O)OC(C)(C)C)CC4)cc3)cc(C3CCCN(C(=O)CCn4cccn4)C3)c(F)c2[nH]1]

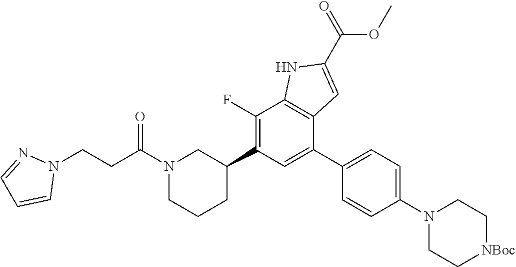 [SMILES: COC(=O)c1cc2c(-c3ccc(N4CCN(C(=O)OC(C)(C)C)CC4)cc3)cc([C@H]3CCCN(C(=O)CCn4cccn4)C3)c(F)c2[nH]1]

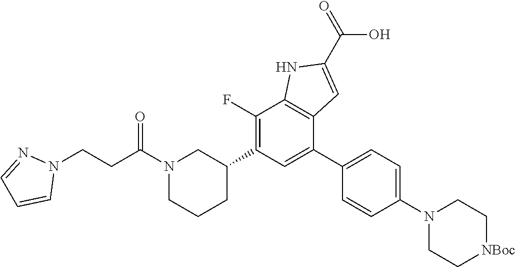 [SMILES: CC(C)(C)OC(=O)N1CCN(c2ccc(-c3cc([C@@H]4CCCN(C(=O)CCn5cccn5)C4)c(F)c4[nH]c(C(=O)O)cc34)cc2)CC1]

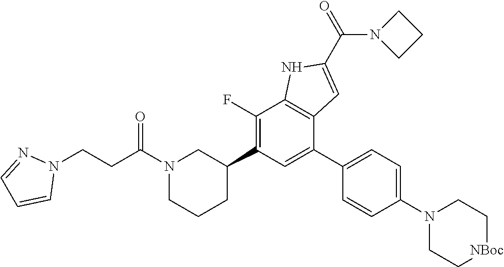 [SMILES: CC(C)(C)OC(=O)N1CCN(c2ccc(-c3cc([C@H]4CCCN(C(=O)CCn5cccn5)C4)c(F)c4[nH]c(C(=O)N5CCC5)cc34)cc2)CC1]

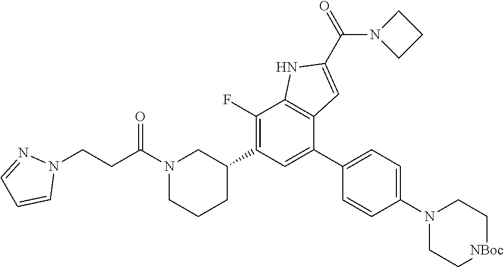 [SMILES: CC(C)(C)OC(=O)N1CCN(c2ccc(-c3cc([C@@H]4CCCN(C(=O)CCn5cccn5)C4)c(F)c4[nH]c(C(=O)N5CCC5)cc34)cc2)CC1]

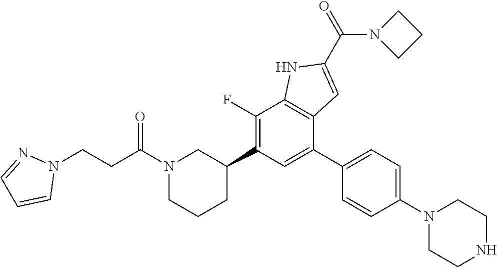 [SMILES: O=C(CCn1cccn1)N1CCC[C@H](c2cc(-c3ccc(N4CCNCC4)cc3)c3cc(C(=O)N4CCC4)[nH]c3c2F)C1]

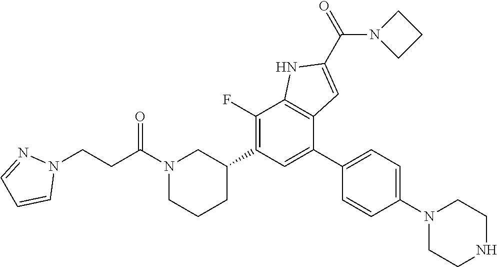 [SMILES: O=C(CCn1cccn1)N1CCC[C@@H](c2cc(-c3ccc(N4CCNCC4)cc3)c3cc(C(=O)N4CCC4)[nH]c3c2F)C1]

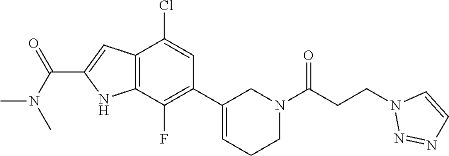 [SMILES: CN(C)C(=O)c1cc2c(Cl)cc(C3=CCCN(C(=O)CCn4ccnn4)C3)c(F)c2[nH]1]

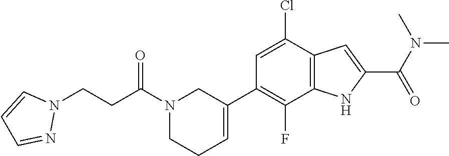 [SMILES: CN(C)C(=O)c1cc2c(Cl)cc(C3=CCCN(C(=O)CCn4cccn4)C3)c(F)c2[nH]1]

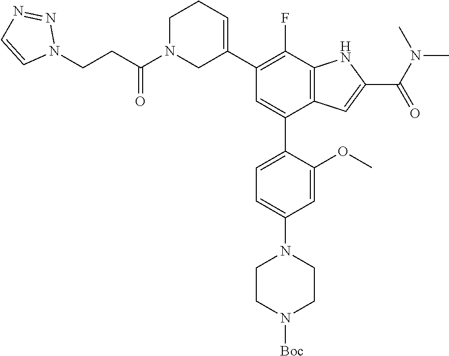 [SMILES: COc1cc(N2CCN(C(=O)OC(C)(C)C)CC2)ccc1-c1cc(C2=CCCN(C(=O)CCn3ccnn3)C2)c(F)c2[nH]c(C(=O)N(C)C)cc12]

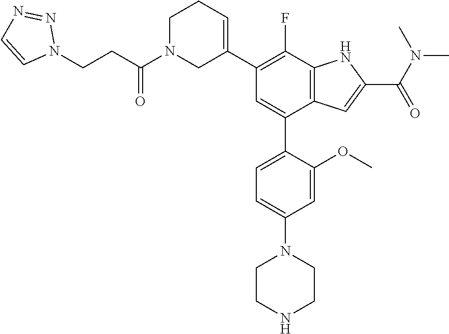 [SMILES: COc1cc(N2CCNCC2)ccc1-c1cc(C2=CCCN(C(=O)CCn3ccnn3)C2)c(F)c2[nH]c(C(=O)N(C)C)cc12]

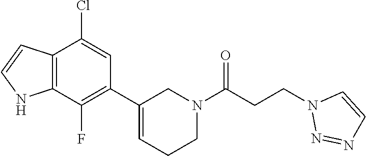 [SMILES: O=C(CCn1ccnn1)N1CCC=C(c2cc(Cl)c3cc[nH]c3c2F)C1]

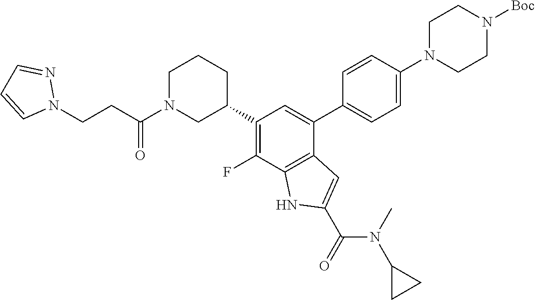 [SMILES: CN(C(=O)c1cc2c(-c3ccc(N4CCN(C(=O)OC(C)(C)C)CC4)cc3)cc([C@H]3CCCN(C(=O)CCn4cccn4)C3)c(F)c2[nH]1)C1CC1]

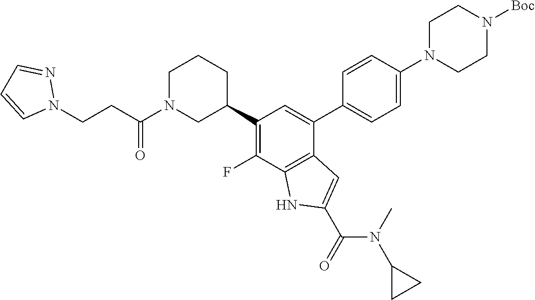 [SMILES: CN(C(=O)c1cc2c(-c3ccc(N4CCN(C(=O)OC(C)(C)C)CC4)cc3)cc([C@@H]3CCCN(C(=O)CCn4cccn4)C3)c(F)c2[nH]1)C1CC1]

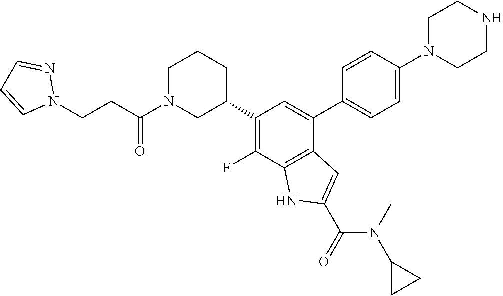 [SMILES: CN(C(=O)c1cc2c(-c3ccc(N4CCNCC4)cc3)cc([C@H]3CCCN(C(=O)CCn4cccn4)C3)c(F)c2[nH]1)C1CC1]

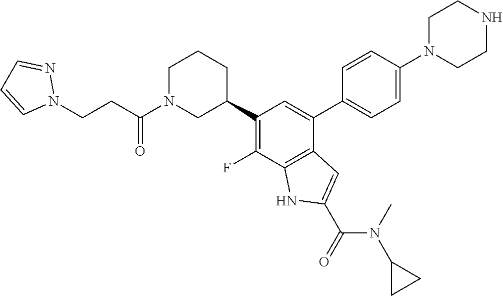 [SMILES: CN(C(=O)c1cc2c(-c3ccc(N4CCNCC4)cc3)cc([C@@H]3CCCN(C(=O)CCn4cccn4)C3)c(F)c2[nH]1)C1CC1]

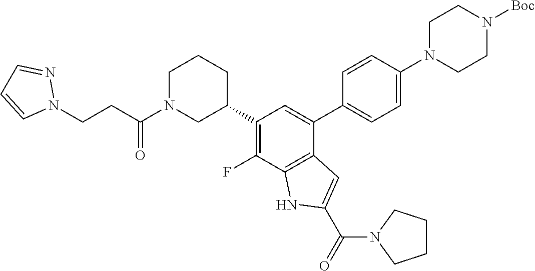 [SMILES: CC(C)(C)OC(=O)N1CCN(c2ccc(-c3cc([C@H]4CCCN(C(=O)CCn5cccn5)C4)c(F)c4[nH]c(C(=O)N5CCCC5)cc34)cc2)CC1]

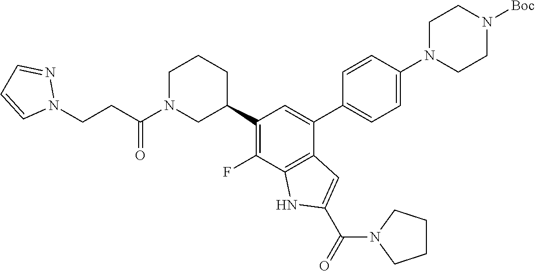 [SMILES: CC(C)(C)OC(=O)N1CCN(c2ccc(-c3cc([C@@H]4CCCN(C(=O)CCn5cccn5)C4)c(F)c4[nH]c(C(=O)N5CCCC5)cc34)cc2)CC1]

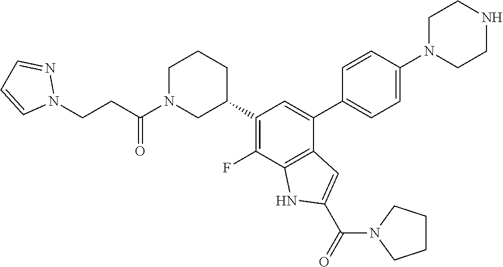 [SMILES: O=C(CCn1cccn1)N1CCC[C@H](c2cc(-c3ccc(N4CCNCC4)cc3)c3cc(C(=O)N4CCCC4)[nH]c3c2F)C1]

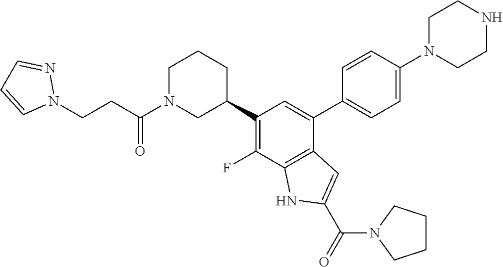 [SMILES: O=C(CCn1cccn1)N1CCC[C@@H](c2cc(-c3ccc(N4CCNCC4)cc3)c3cc(C(=O)N4CCCC4)[nH]c3c2F)C1]

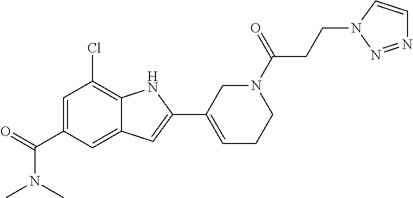 [SMILES: CN(C)C(=O)c1cc(Cl)c2[nH]c(C3=CCCN(C(=O)CCn4ccnn4)C3)cc2c1]

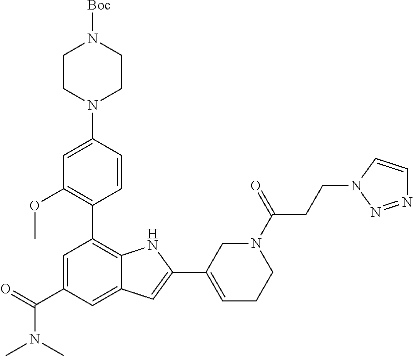 [SMILES: COc1cc(N2CCN(C(=O)OC(C)(C)C)CC2)ccc1-c1cc(C(=O)N(C)C)cc2cc(C3=CCCN(C(=O)CCn4ccnn4)C3)[nH]c12]

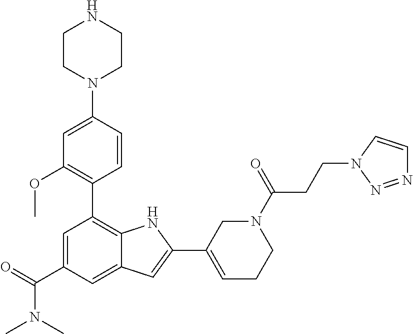 [SMILES: COc1cc(N2CCNCC2)ccc1-c1cc(C(=O)N(C)C)cc2cc(C3=CCCN(C(=O)CCn4ccnn4)C3)[nH]c12]

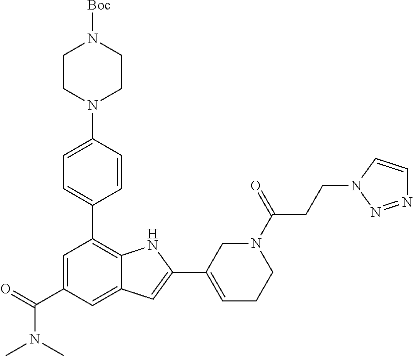 [SMILES: CN(C)C(=O)c1cc(-c2ccc(N3CCN(C(=O)OC(C)(C)C)CC3)cc2)c2[nH]c(C3=CCCN(C(=O)CCn4ccnn4)C3)cc2c1]

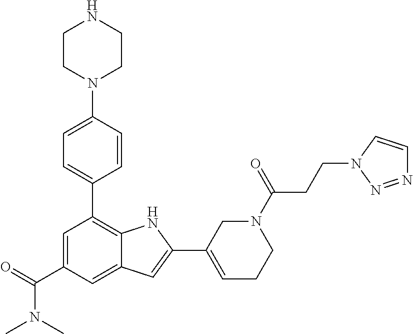 [SMILES: CN(C)C(=O)c1cc(-c2ccc(N3CCNCC3)cc2)c2[nH]c(C3=CCCN(C(=O)CCn4ccnn4)C3)cc2c1]

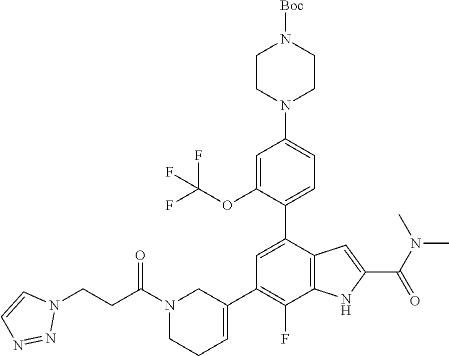 [SMILES: CN(C)C(=O)c1cc2c(-c3ccc(N4CCN(C(=O)OC(C)(C)C)CC4)cc3OC(F)(F)F)cc(C3=CCCN(C(=O)CCn4ccnn4)C3)c(F)c2[nH]1]

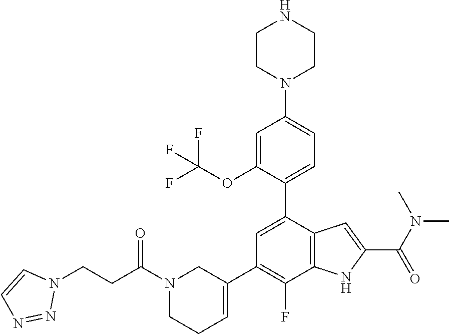 [SMILES: CN(C)C(=O)c1cc2c(-c3ccc(N4CCNCC4)cc3OC(F)(F)F)cc(C3=CCCN(C(=O)CCn4ccnn4)C3)c(F)c2[nH]1]

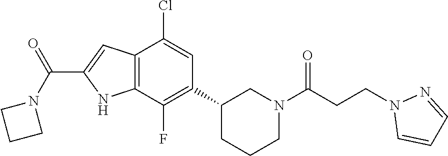 [SMILES: O=C(CCn1cccn1)N1CCC[C@H](c2cc(Cl)c3cc(C(=O)N4CCC4)[nH]c3c2F)C1]

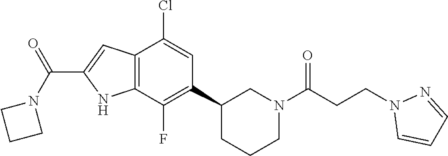 [SMILES: O=C(CCn1cccn1)N1CCC[C@@H](c2cc(Cl)c3cc(C(=O)N4CCC4)[nH]c3c2F)C1]

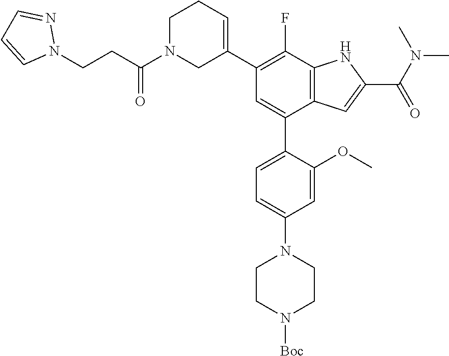 [SMILES: COc1cc(N2CCN(C(=O)OC(C)(C)C)CC2)ccc1-c1cc(C2=CCCN(C(=O)CCn3cccn3)C2)c(F)c2[nH]c(C(=O)N(C)C)cc12]

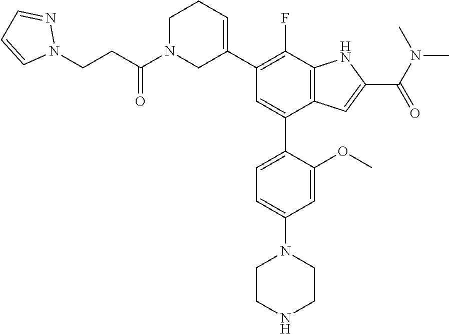 [SMILES: COc1cc(N2CCNCC2)ccc1-c1cc(C2=CCCN(C(=O)CCn3cccn3)C2)c(F)c2[nH]c(C(=O)N(C)C)cc12]

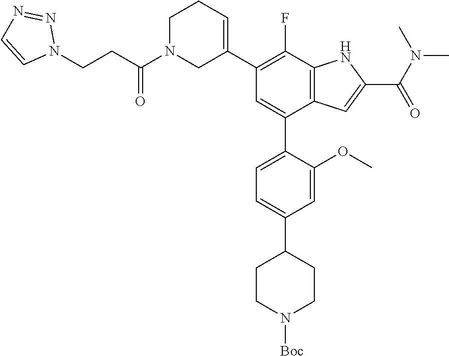 [SMILES: COc1cc(C2CCN(C(=O)OC(C)(C)C)CC2)ccc1-c1cc(C2=CCCN(C(=O)CCn3ccnn3)C2)c(F)c2[nH]c(C(=O)N(C)C)cc12]

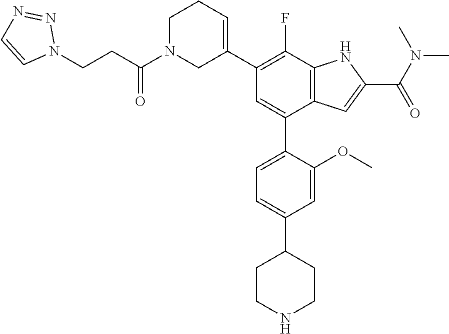 [SMILES: COc1cc(C2CCNCC2)ccc1-c1cc(C2=CCCN(C(=O)CCn3ccnn3)C2)c(F)c2[nH]c(C(=O)N(C)C)cc12]

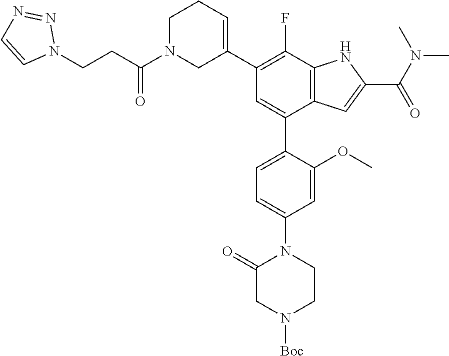 [SMILES: COc1cc(N2CCN(C(=O)OC(C)(C)C)CC2=O)ccc1-c1cc(C2=CCCN(C(=O)CCn3ccnn3)C2)c(F)c2[nH]c(C(=O)N(C)C)cc12]

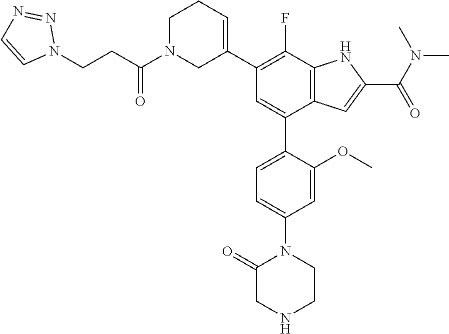 [SMILES: COc1cc(N2CCNCC2=O)ccc1-c1cc(C2=CCCN(C(=O)CCn3ccnn3)C2)c(F)c2[nH]c(C(=O)N(C)C)cc12]

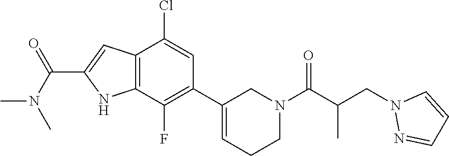 [SMILES: CC(Cn1cccn1)C(=O)N1CCC=C(c2cc(Cl)c3cc(C(=O)N(C)C)[nH]c3c2F)C1]

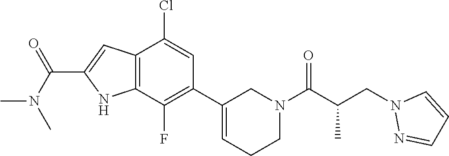 [SMILES: C[C@@H](Cn1cccn1)C(=O)N1CCC=C(c2cc(Cl)c3cc(C(=O)N(C)C)[nH]c3c2F)C1]

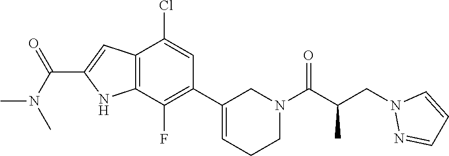 [SMILES: C[C@H](Cn1cccn1)C(=O)N1CCC=C(c2cc(Cl)c3cc(C(=O)N(C)C)[nH]c3c2F)C1]

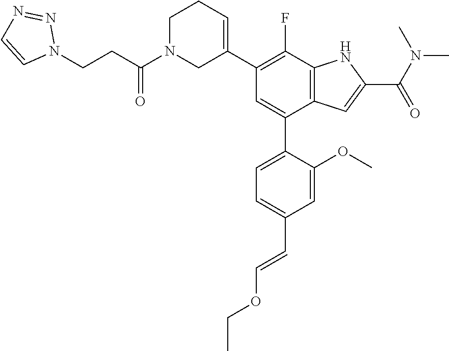 [SMILES: CCO/C=C/c1ccc(-c2cc(C3=CCCN(C(=O)CCn4ccnn4)C3)c(F)c3[nH]c(C(=O)N(C)C)cc23)c(OC)c1]

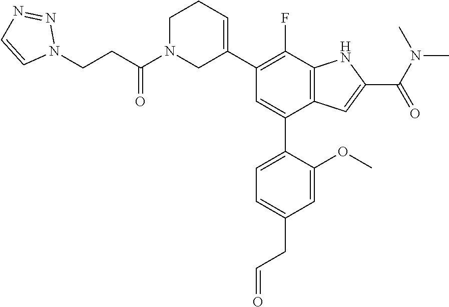 [SMILES: COc1cc(CC=O)ccc1-c1cc(C2=CCCN(C(=O)CCn3ccnn3)C2)c(F)c2[nH]c(C(=O)N(C)C)cc12]

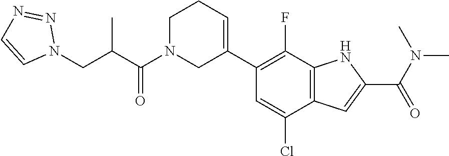 [SMILES: CC(Cn1ccnn1)C(=O)N1CCC=C(c2cc(Cl)c3cc(C(=O)N(C)C)[nH]c3c2F)C1]

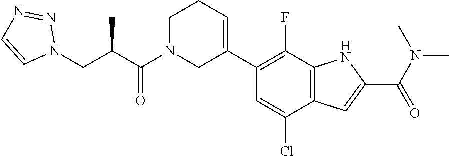 [SMILES: C[C@H](Cn1ccnn1)C(=O)N1CCC=C(c2cc(Cl)c3cc(C(=O)N(C)C)[nH]c3c2F)C1]

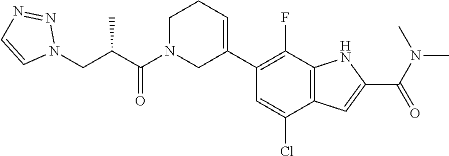 [SMILES: C[C@@H](Cn1ccnn1)C(=O)N1CCC=C(c2cc(Cl)c3cc(C(=O)N(C)C)[nH]c3c2F)C1]

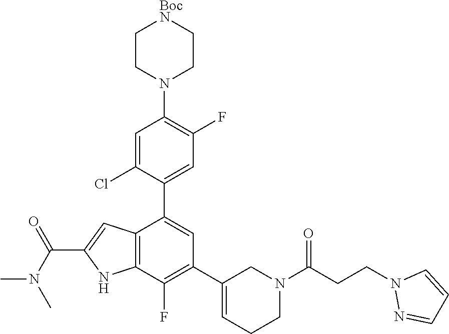 [SMILES: CN(C)C(=O)c1cc2c(-c3cc(F)c(N4CCN(C(=O)OC(C)(C)C)CC4)cc3Cl)cc(C3=CCCN(C(=O)CCn4cccn4)C3)c(F)c2[nH]1]

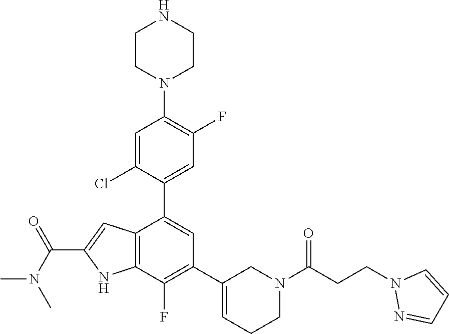 [SMILES: CN(C)C(=O)c1cc2c(-c3cc(F)c(N4CCNCC4)cc3Cl)cc(C3=CCCN(C(=O)CCn4cccn4)C3)c(F)c2[nH]1]

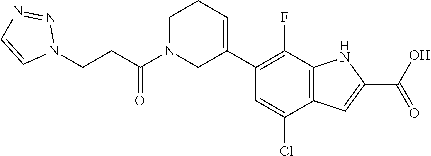 [SMILES: O=C(O)c1cc2c(Cl)cc(C3=CCCN(C(=O)CCn4ccnn4)C3)c(F)c2[nH]1]

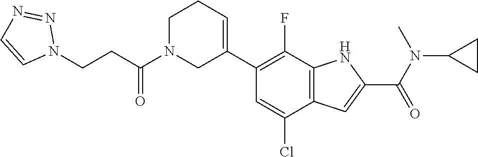 [SMILES: CN(C(=O)c1cc2c(Cl)cc(C3=CCCN(C(=O)CCn4ccnn4)C3)c(F)c2[nH]1)C1CC1]

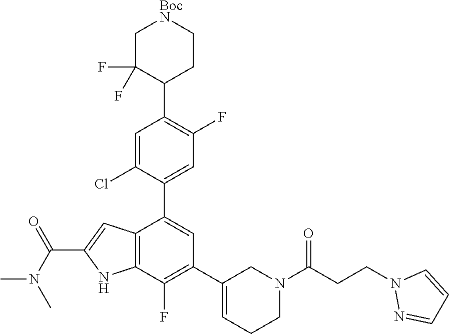 [SMILES: CN(C)C(=O)c1cc2c(-c3cc(F)c(C4CCN(C(=O)OC(C)(C)C)CC4(F)F)cc3Cl)cc(C3=CCCN(C(=O)CCn4cccn4)C3)c(F)c2[nH]1]

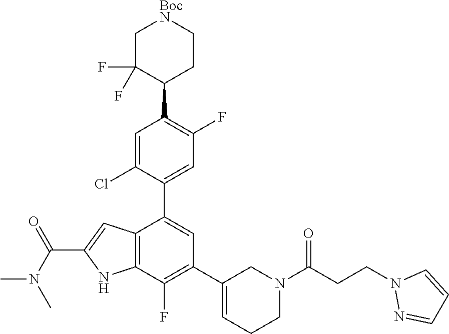 [SMILES: CN(C)C(=O)c1cc2c(-c3cc(F)c([C@@H]4CCN(C(=O)OC(C)(C)C)CC4(F)F)cc3Cl)cc(C3=CCCN(C(=O)CCn4cccn4)C3)c(F)c2[nH]1]

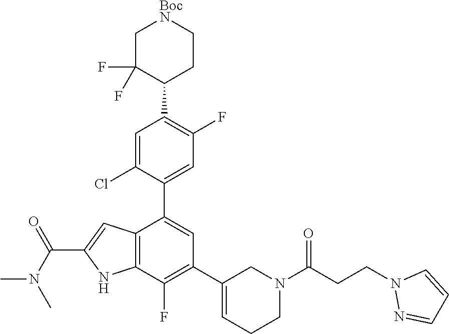 [SMILES: CN(C)C(=O)c1cc2c(-c3cc(F)c([C@H]4CCN(C(=O)OC(C)(C)C)CC4(F)F)cc3Cl)cc(C3=CCCN(C(=O)CCn4cccn4)C3)c(F)c2[nH]1]

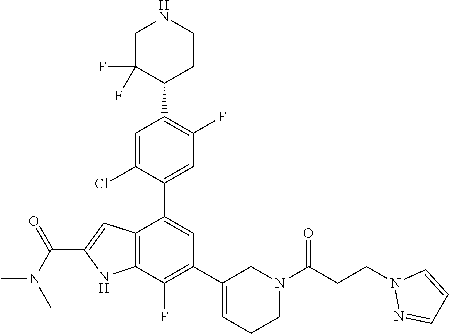 [SMILES: CN(C)C(=O)c1cc2c(-c3cc(F)c([C@H]4CCNCC4(F)F)cc3Cl)cc(C3=CCCN(C(=O)CCn4cccn4)C3)c(F)c2[nH]1]

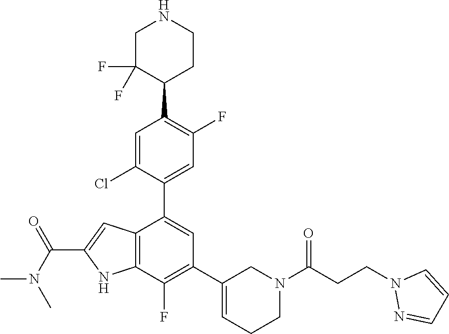 [SMILES: CN(C)C(=O)c1cc2c(-c3cc(F)c([C@@H]4CCNCC4(F)F)cc3Cl)cc(C3=CCCN(C(=O)CCn4cccn4)C3)c(F)c2[nH]1]

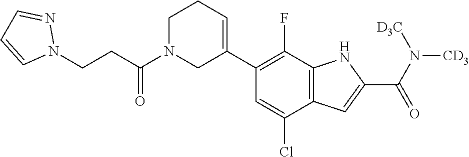 [SMILES: [2H]C([2H])([2H])N(C(=O)c1cc2c(Cl)cc(C3=CCCN(C(=O)CCn4cccn4)C3)c(F)c2[nH]1)C([2H])([2H])[2H]]

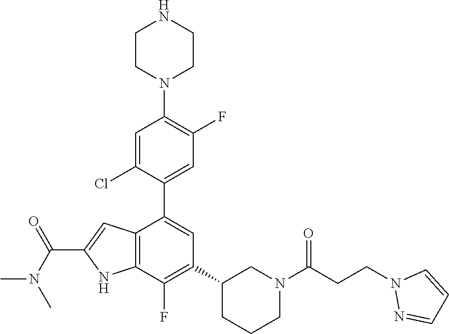 [SMILES: CN(C)C(=O)c1cc2c(-c3cc(F)c(N4CCNCC4)cc3Cl)cc([C@H]3CCCN(C(=O)CCn4cccn4)C3)c(F)c2[nH]1]

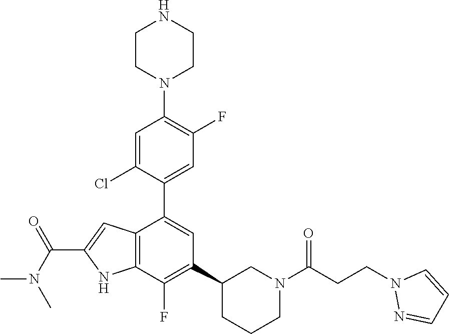 [SMILES: CN(C)C(=O)c1cc2c(-c3cc(F)c(N4CCNCC4)cc3Cl)cc([C@@H]3CCCN(C(=O)CCn4cccn4)C3)c(F)c2[nH]1]

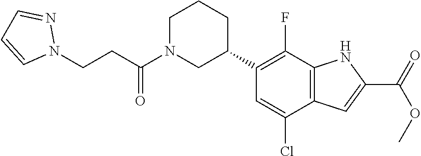 [SMILES: COC(=O)c1cc2c(Cl)cc([C@H]3CCCN(C(=O)CCn4cccn4)C3)c(F)c2[nH]1]

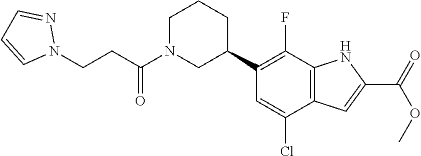 [SMILES: COC(=O)c1cc2c(Cl)cc([C@@H]3CCCN(C(=O)CCn4cccn4)C3)c(F)c2[nH]1]

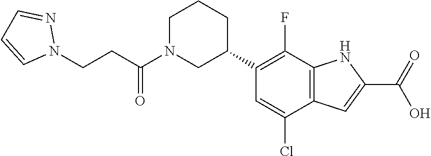 [SMILES: O=C(O)c1cc2c(Cl)cc([C@H]3CCCN(C(=O)CCn4cccn4)C3)c(F)c2[nH]1]

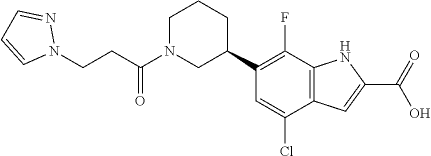 [SMILES: O=C(O)c1cc2c(Cl)cc([C@@H]3CCCN(C(=O)CCn4cccn4)C3)c(F)c2[nH]1]

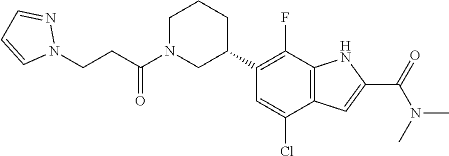 [SMILES: CN(C)C(=O)c1cc2c(Cl)cc([C@H]3CCCN(C(=O)CCn4cccn4)C3)c(F)c2[nH]1]

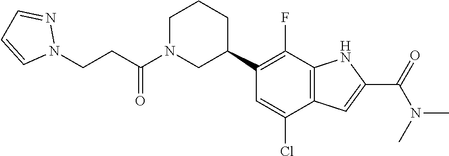 [SMILES: CN(C)C(=O)c1cc2c(Cl)cc([C@@H]3CCCN(C(=O)CCn4cccn4)C3)c(F)c2[nH]1]

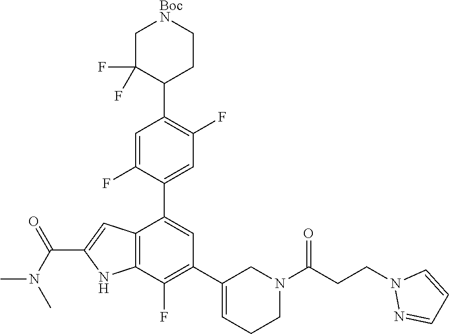 [SMILES: CN(C)C(=O)c1cc2c(-c3cc(F)c(C4CCN(C(=O)OC(C)(C)C)CC4(F)F)cc3F)cc(C3=CCCN(C(=O)CCn4cccn4)C3)c(F)c2[nH]1]

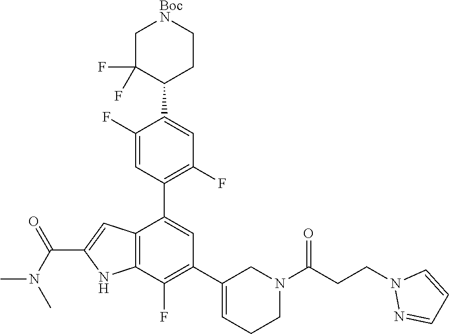 [SMILES: CN(C)C(=O)c1cc2c(-c3cc(F)c([C@H]4CCN(C(=O)OC(C)(C)C)CC4(F)F)cc3F)cc(C3=CCCN(C(=O)CCn4cccn4)C3)c(F)c2[nH]1]

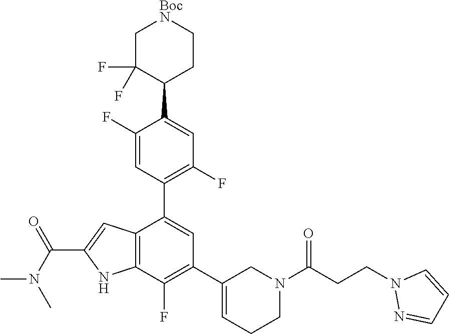 [SMILES: CN(C)C(=O)c1cc2c(-c3cc(F)c([C@@H]4CCN(C(=O)OC(C)(C)C)CC4(F)F)cc3F)cc(C3=CCCN(C(=O)CCn4cccn4)C3)c(F)c2[nH]1]

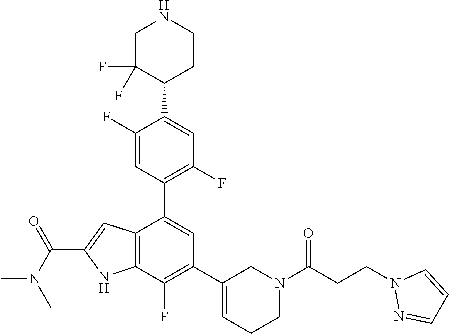 [SMILES: CN(C)C(=O)c1cc2c(-c3cc(F)c([C@H]4CCNCC4(F)F)cc3F)cc(C3=CCCN(C(=O)CCn4cccn4)C3)c(F)c2[nH]1]

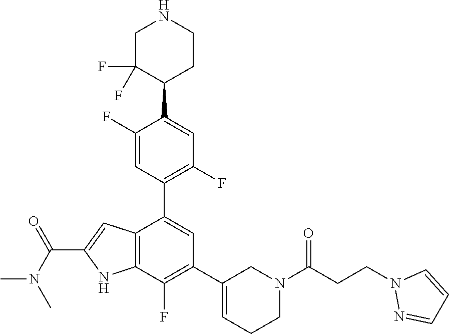 [SMILES: CN(C)C(=O)c1cc2c(-c3cc(F)c([C@@H]4CCNCC4(F)F)cc3F)cc(C3=CCCN(C(=O)CCn4cccn4)C3)c(F)c2[nH]1]

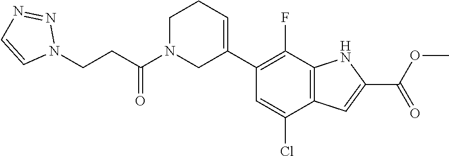 [SMILES: COC(=O)c1cc2c(Cl)cc(C3=CCCN(C(=O)CCn4ccnn4)C3)c(F)c2[nH]1]

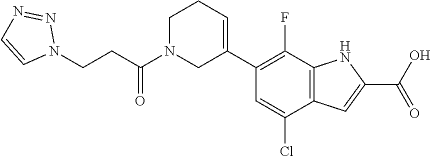 [SMILES: O=C(O)c1cc2c(Cl)cc(C3=CCCN(C(=O)CCn4ccnn4)C3)c(F)c2[nH]1]

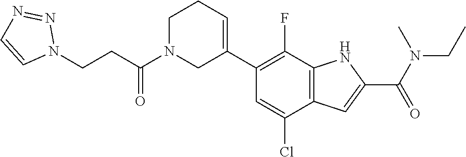 [SMILES: CCN(C)C(=O)c1cc2c(Cl)cc(C3=CCCN(C(=O)CCn4ccnn4)C3)c(F)c2[nH]1]

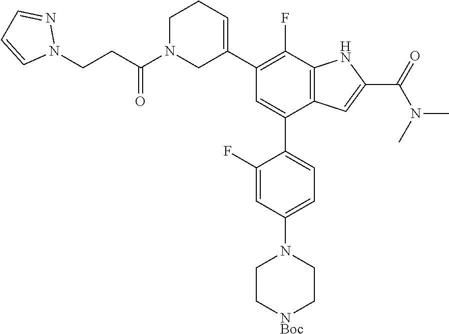 [SMILES: CN(C)C(=O)c1cc2c(-c3ccc(N4CCN(C(=O)OC(C)(C)C)CC4)cc3F)cc(C3=CCCN(C(=O)CCn4cccn4)C3)c(F)c2[nH]1]

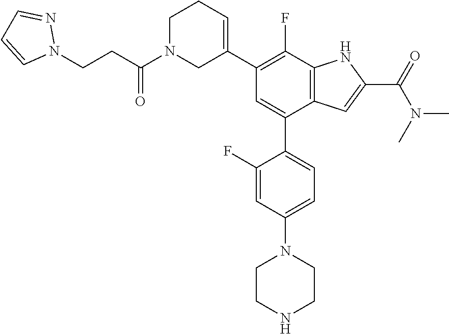 [SMILES: CN(C)C(=O)c1cc2c(-c3ccc(N4CCNCC4)cc3F)cc(C3=CCCN(C(=O)CCn4cccn4)C3)c(F)c2[nH]1]

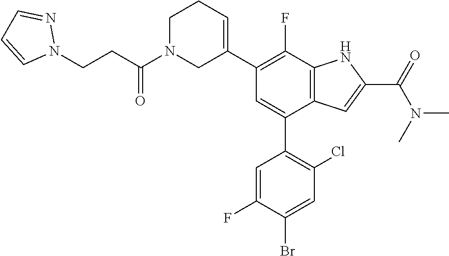 [SMILES: CN(C)C(=O)c1cc2c(-c3cc(F)c(Br)cc3Cl)cc(C3=CCCN(C(=O)CCn4cccn4)C3)c(F)c2[nH]1]

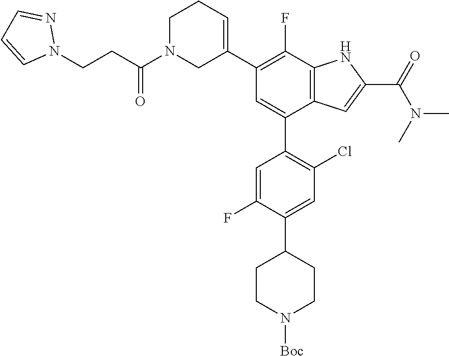 [SMILES: CN(C)C(=O)c1cc2c(-c3cc(F)c(C4CCN(C(=O)OC(C)(C)C)CC4)cc3Cl)cc(C3=CCCN(C(=O)CCn4cccn4)C3)c(F)c2[nH]1]

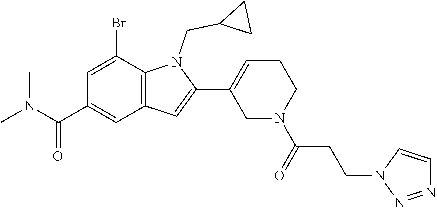 [SMILES: CN(C)C(=O)c1cc(Br)c2c(c1)cc(C1=CCCN(C(=O)CCn3ccnn3)C1)n2CC1CC1]

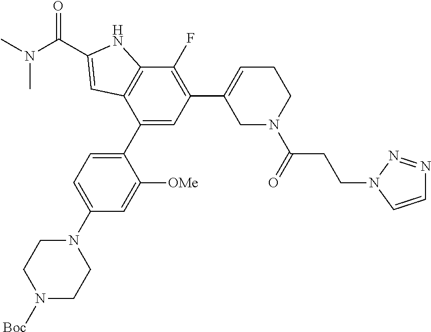 [SMILES: COc1cc(N2CCN(C(=O)OC(C)(C)C)CC2)ccc1-c1cc(C2=CCCN(C(=O)CCn3ccnn3)C2)c(F)c2[nH]c(C(=O)N(C)C)cc12]

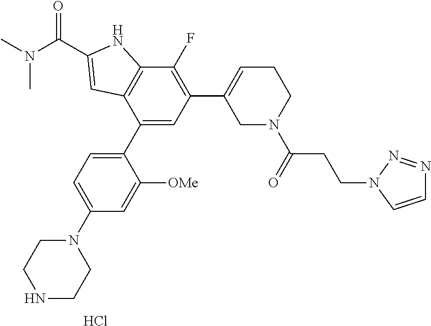 [SMILES: COc1cc(N2CCNCC2)ccc1-c1cc(C2=CCCN(C(=O)CCn3ccnn3)C2)c(F)c2[nH]c(C(=O)N(C)C)cc12 ; Cl]

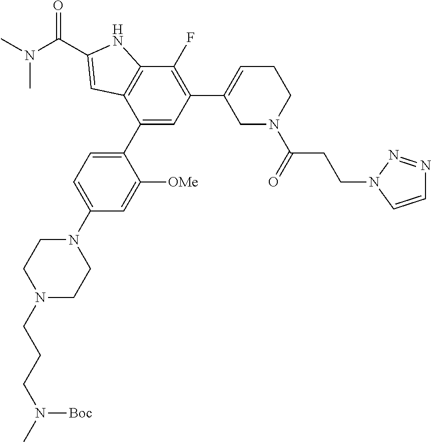 [SMILES: COc1cc(N2CCN(CCCN(C)C(=O)OC(C)(C)C)CC2)ccc1-c1cc(C2=CCCN(C(=O)CCn3ccnn3)C2)c(F)c2[nH]c(C(=O)N(C)C)cc12]

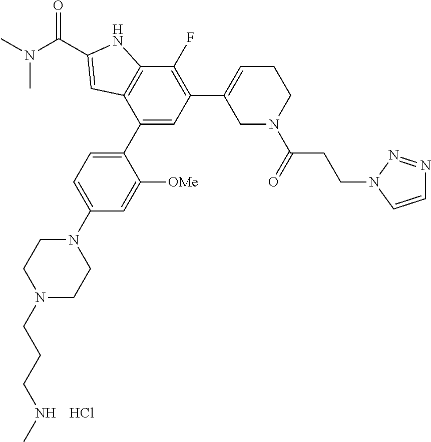 [SMILES: CNCCCN1CCN(c2ccc(-c3cc(C4=CCCN(C(=O)CCn5ccnn5)C4)c(F)c4[nH]c(C(=O)N(C)C)cc34)c(OC)c2)CC1 ; Cl]

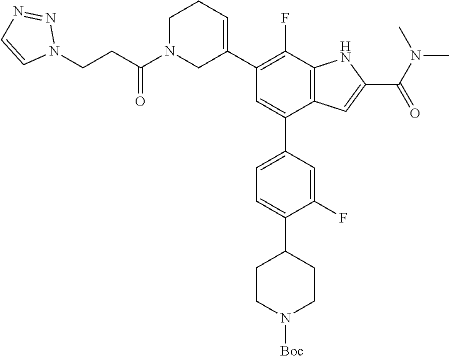 [SMILES: CN(C)C(=O)c1cc2c(-c3ccc(C4CCN(C(=O)OC(C)(C)C)CC4)c(F)c3)cc(C3=CCCN(C(=O)CCn4ccnn4)C3)c(F)c2[nH]1]

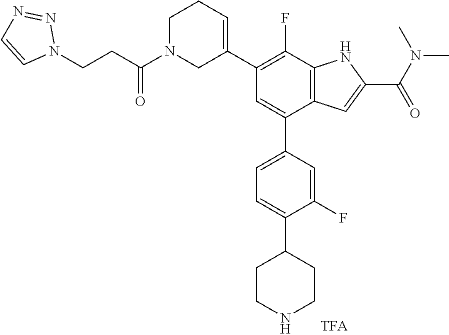 [SMILES: CN(C)C(=O)c1cc2c(-c3ccc(C4CCNCC4)c(F)c3)cc(C3=CCCN(C(=O)CCn4ccnn4)C3)c(F)c2[nH]1]

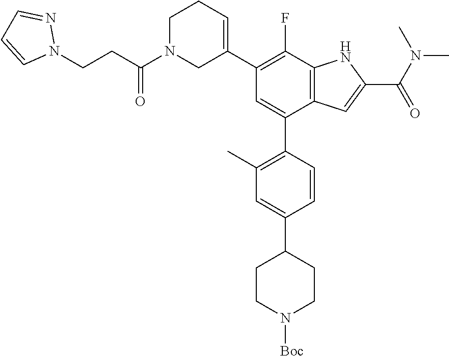 [SMILES: Cc1cc(C2CCN(C(=O)OC(C)(C)C)CC2)ccc1-c1cc(C2=CCCN(C(=O)CCn3cccn3)C2)c(F)c2[nH]c(C(=O)N(C)C)cc12]

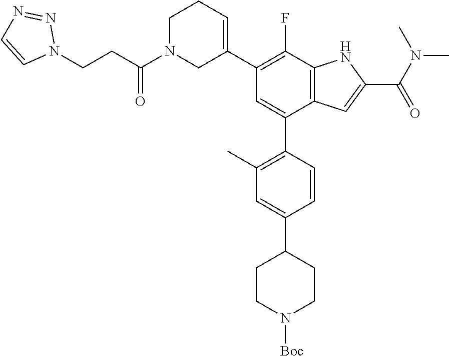 [SMILES: Cc1cc(C2CCN(C(=O)OC(C)(C)C)CC2)ccc1-c1cc(C2=CCCN(C(=O)CCn3ccnn3)C2)c(F)c2[nH]c(C(=O)N(C)C)cc12]

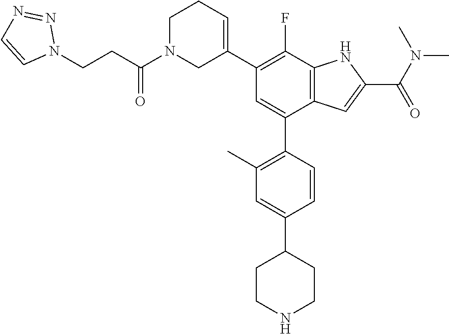 [SMILES: Cc1cc(C2CCNCC2)ccc1-c1cc(C2=CCCN(C(=O)CCn3ccnn3)C2)c(F)c2[nH]c(C(=O)N(C)C)cc12]

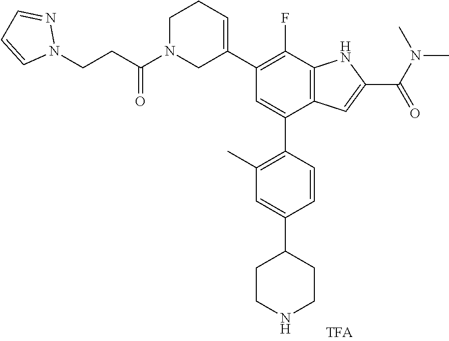 [SMILES: Cc1cc(C2CCNCC2)ccc1-c1cc(C2=CCCN(C(=O)CCn3cccn3)C2)c(F)c2[nH]c(C(=O)N(C)C)cc12]

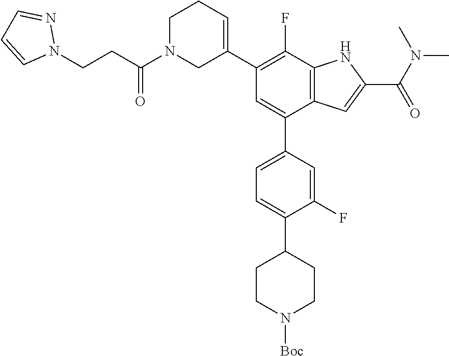 [SMILES: CN(C)C(=O)c1cc2c(-c3ccc(C4CCN(C(=O)OC(C)(C)C)CC4)c(F)c3)cc(C3=CCCN(C(=O)CCn4cccn4)C3)c(F)c2[nH]1]

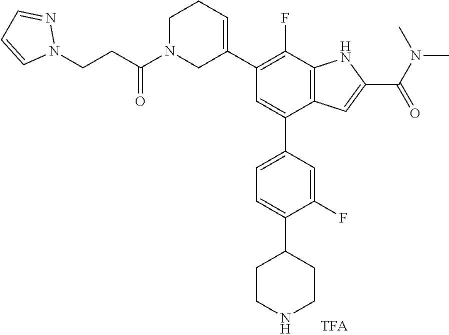 [SMILES: CN(C)C(=O)c1cc2c(-c3ccc(C4CCNCC4)c(F)c3)cc(C3=CCCN(C(=O)CCn4cccn4)C3)c(F)c2[nH]1]

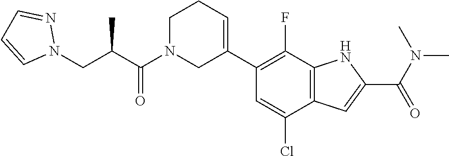 [SMILES: C[C@H](Cn1cccn1)C(=O)N1CCC=C(c2cc(Cl)c3cc(C(=O)N(C)C)[nH]c3c2F)C1]

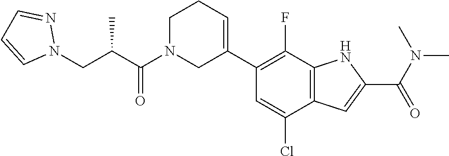 [SMILES: C[C@@H](Cn1cccn1)C(=O)N1CCC=C(c2cc(Cl)c3cc(C(=O)N(C)C)[nH]c3c2F)C1]

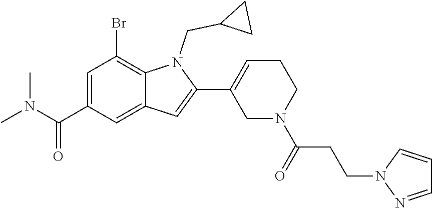 [SMILES: CN(C)C(=O)c1cc(Br)c2c(c1)cc(C1=CCCN(C(=O)CCn3cccn3)C1)n2CC1CC1]

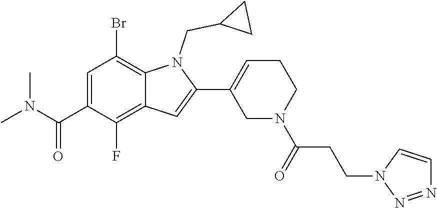 [SMILES: CN(C)C(=O)c1cc(Br)c2c(cc(C3=CCCN(C(=O)CCn4ccnn4)C3)n2CC2CC2)c1F]

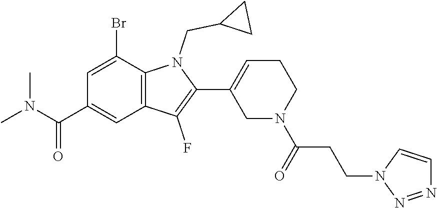 [SMILES: CN(C)C(=O)c1cc(Br)c2c(c1)c(F)c(C1=CCCN(C(=O)CCn3ccnn3)C1)n2CC1CC1]

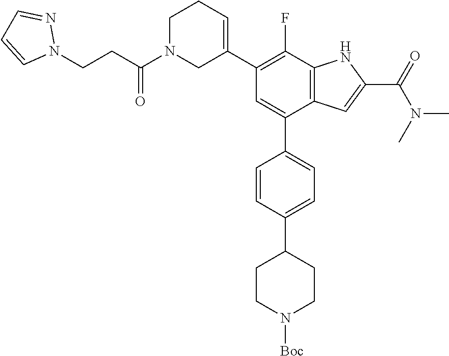 [SMILES: CN(C)C(=O)c1cc2c(-c3ccc(C4CCN(C(=O)OC(C)(C)C)CC4)cc3)cc(C3=CCCN(C(=O)CCn4cccn4)C3)c(F)c2[nH]1]

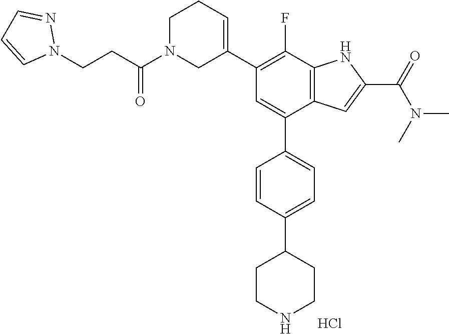 [SMILES: CN(C)C(=O)c1cc2c(-c3ccc(C4CCNCC4)cc3)cc(C3=CCCN(C(=O)CCn4cccn4)C3)c(F)c2[nH]1 ; Cl]

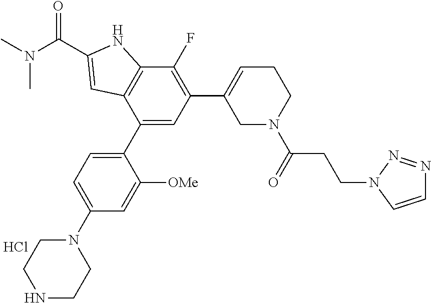 [SMILES: COc1cc(N2CCNCC2)ccc1-c1cc(C2=CCCN(C(=O)CCn3ccnn3)C2)c(F)c2[nH]c(C(=O)N(C)C)cc12 ; Cl]

or a salt or free base/acid thereof.

As described above and defined herein, Ring W and its R^{w} substituents is

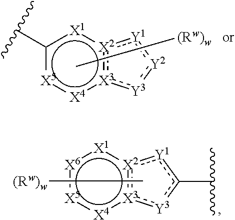

wherein each of X¹, X⁴, X⁵ and X⁶ is independently CH, CR^{w}, or N; each of X² and X³ is independently C or N, wherein at most one of X¹, X², X³, X⁴, X⁵ and X⁶ is N; each of Y¹, Y², and Y³ is independently CH, CR^{w}, CH₂, CH(R^{w}), C(R^{w})₂, NH, NR^{w}, N, O, or S, wherein at least one of Y² and Y³ is CH, CR^{w}, CH₂, CH(R^{w}), or C(R^{w})₂; and each [symbol] represents a single bond or a double bond.

In some embodiments, Ring W and its R^{w} substituents is

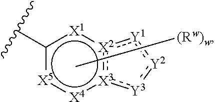

wherein each of X¹, X⁴, and X⁵ is independently CH, CR^{w}, or N; each of X² and X³ is independently C or N, wherein at most one of X¹, X², X³, X⁴, and X⁵ is N; each of Y¹, Y², and Y³ is independently CH, CR^{w}, CH₂, CH(R^{w}), C(R^{w})₂, NH, NR^{w}, N, O, or S, wherein at least one of Y² and Y³ is CH, CR^{w}, CH₂, CH(R^{w}), or C(R^{w})₂; and each [symbol] represents a single bond or a double bond.

In some embodiments, Ring W and its R^{w} substituents is

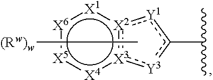

wherein each of X¹, X⁴, X⁵ and X⁶ is independently CH, CR^{w}, or N; each of X² and X³ is independently C or N, wherein at most one of X¹, X², X³, X⁴, X⁵ and X⁶ is N; each of Y¹, Y², and Y³ is independently CH, CR^{w}, CH₂, CH(R^{w}), C(R^{w})₂, NH, NR^{w}, N, O, or S, wherein at least one of Y² and Y³ is CH, CR^{w}, CH₂, CH(R^{w}), or C(R^{w})₂; and each [symbol] represents a single bond or a double bond.

In some embodiments, the ring containing X², X³, Y¹, Y², and Y³ is aromatic or heteroaromatic, i.e., the definitions of Y, Y², Y³, and [symbol] are selected to satisfy valency of an aromatic or heteroaromatic ring. In some embodiments, the ring containing X², X³, Y¹, Y², and Y³ is not aromatic or heteroaromatic, i.e., the definitions of Y¹, Y², Y³, and [symbol] are selected to satisfy valency of a partially unsaturated ring.

In some embodiments, Ring W and its R^{w} substituents is:

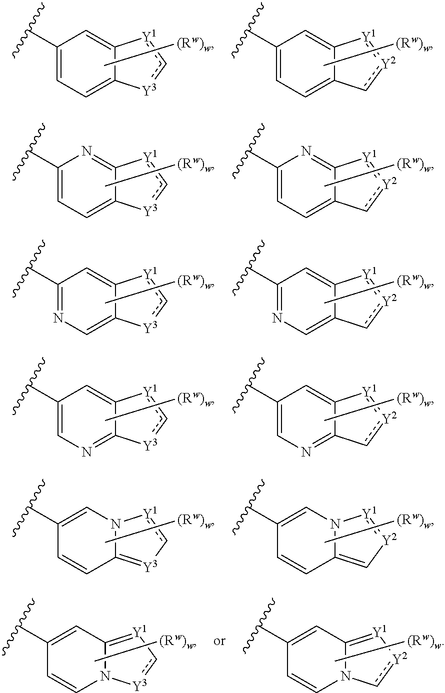

In some embodiments, Ring W and its R^{w} substituents is:

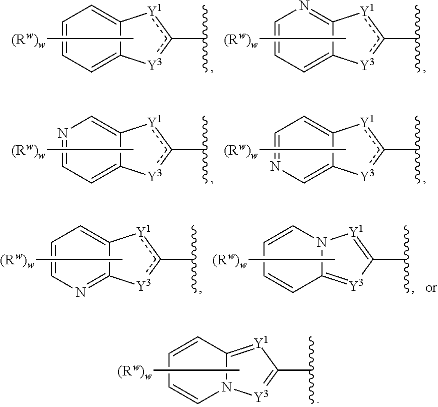

As defined above and described herein, each of X¹, X⁴, X⁵, and X⁶ is independently CH, CR^{w}, or N. In some embodiments, each of X¹, X⁴, X⁵ and X⁶ is CH or CR^{w}. In some embodiments, each of X¹, X⁴, and X⁵ is CH or CR^{w}. In some embodiments, X¹ is CH. In some embodiments, X¹ is CR^{w}. In some embodiments, X¹ is N. In some embodiments, X⁴ is CH. In some embodiments, X⁴ is CR^{w}. In some embodiments, X⁴ is N. In some embodiments, X⁵ is CH. In some embodiments, X⁵ is CR^{w}. In some embodiments, X⁵ is N. In some embodiments, X⁶ is CH. In some embodiments, X⁶ is CR^{w}. In some embodiments, X⁶ is N.

As defined above and described herein, each of X² and X³ is independently C or N. In some embodiments, both of X² and X³ are C. In some embodiments, X² is C. In some embodiments, X² is N. In some embodiments, X³ is C. In some embodiments, X³ is N.

In some embodiments, X¹, X⁴, and X⁵ are CH or CR^{w}, and X² and X³ are C. In some embodiments, X¹ is N, X² and X³ are C, and X⁴ and X⁵ are CH or CR^{w}. In some embodiments, X¹, X⁴, and X⁵ are CH or CR^{w}, X² is N, and X³ are C. In some embodiments, X¹, X⁴, and X⁵ are CH or CR^{w}, X² is C, and X³ are N. In some embodiments, X¹ and X⁵ are CH or CR^{w}, X² and X³ are C, and X⁴ is N. In some embodiments, X¹ and X⁴ are CH or CR^{w}, X² and X³ are C, and X⁵ is N.

In some embodiments, X¹, X⁴, X⁵, and X⁶ are CH or CR^{w}, and X² and X³ are C. In some embodiments, X¹ is N, X² and X³ are C, and X⁴, X⁵, and X⁶ are CH or CR^{w}. In some embodiments, X¹, X⁴, X⁵, and X⁶ are CH or CR^{w}, X² is N, and X³ are C. In some embodiments, X¹, X⁴, X⁵, and X⁶ are CH or CR^{w}, X² is C, and X³ are N. In some embodiments, X¹, X⁵, and X⁶ are CH or CR^{w}, X² and X³ are C, and X⁴ is N. In some embodiments, X¹, X⁴, and X⁶ are CH or CR^{w}, X² and X³ are C, and X⁵ is N. In some embodiments, X¹, X⁴, and X⁵ are CH or CR^{w}, X² and X³ are C, and X⁶ is N.

As defined above and described herein, each of Y¹, Y², and Y³ is independently CH, CR^{w}, CH₂, CH(R^{w}), C(R^{w})₂, NH, NR^{w}, N, O, or S, wherein at least one of Y² and Y³ is CH, CR^{w}, CH₂, CH(R^{w}), or C(R^{w})₂. In some embodiments, both of Y² and Y³ are CH, CR^{w}, CH₂, CH(R^{w}), or C(R^{w})₂. In some embodiments, each of Y¹, Y², and Y³ is independently CH, CR^{w}, NH, NR^{w}, N, O, or S, wherein at least one of Y² and Y³ is CH or CR^{w}. In some embodiments, Y¹ is CH. In some embodiments, Y¹ is CR^{w}. In some embodiments, Y¹ is CH₂. In some embodiments, Y¹ is CH(R^{w}). In some embodiments, Y¹ is C(R^{w})₂. In some embodiments, Y¹ is NH. In some embodiments, Y¹ is NR^{w}. In some embodiments, Y¹ is N. In some embodiments, Y¹ is O. In some embodiments, Y¹ is S. In some embodiments, Y² is CH. In some embodiments, Y² is CR^{w}. In some embodiments, Y² is CH₂. In some embodiments, Y² is CH(R^{w}). In some embodiments, Y² is C(R^{w})₂. In some embodiments, Y² is NH. In some embodiments, Y² is NR^{w}. In some embodiments, Y² is N. In some embodiments, Y² is O. In some embodiments, Y² is S. In some embodiments, Y³ is CH. In some embodiments, Y³ is CR^{w}. In some embodiments, Y³ is CH₂. In some embodiments, Y³ is CH(R^{w}). In some embodiments, Y³ is C(R^{w})₂. In some embodiments, Y³ is NH. In some embodiments, Y³ is NR^{w}. In some embodiments, Y³ is N. In some embodiments, Y³ is O. In some embodiments, Y³ is S.

In some embodiments, Y¹ is NH or NR^{w}, and Y^{Z} and Y³ are CH or CR^{w}. In some embodiments, Y¹ is O, and Y² and Y³ are CH or CR^{w}. In some embodiments, Y¹ is S, and Y² and Y³ are CH or CR^{w}.

In some embodiments, Ring W and its R^{w} substituents is

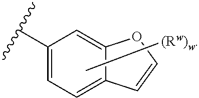

In some embodiments, Ring W and its R^{w} substituents is

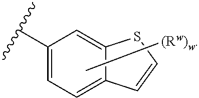

In some embodiments, Ring W and its R^{w} substituents is

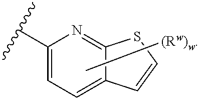

In some embodiments, Ring W and its R^{w} substituents is

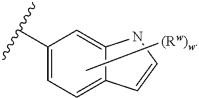

In some embodiments, Ring W and its R^{w} substituents is

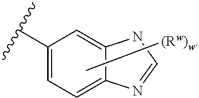

In some embodiments, Ring W and its R^{w} substituents is

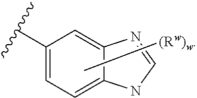

In some embodiments, Ring W and its R^{w} substituents is

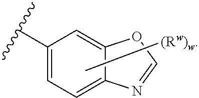

In some embodiments, Ring W and its R^{w} substituents is

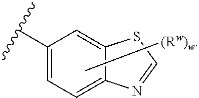

In some embodiments, Ring W and its R^{w} substituents is

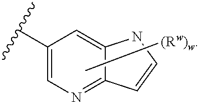

In some embodiments, Ring W and its R^{w} substituents is

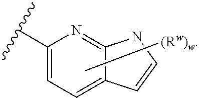

In some embodiments, Ring W and its R^{w} substituents is

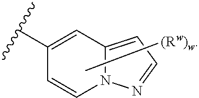

In some embodiments, Ring W and its R^{w} substituents is

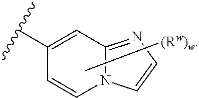

In some embodiments, Ring W and its R^{w} substituents is

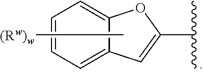

In some embodiments, Ring W and its R^{w} substituents is

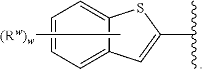

In some embodiments, Ring W and its R^{w} substituents is

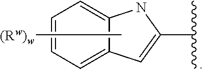

In some embodiments, Ring W and its R^{w} substituents is

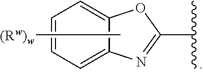

In some embodiments, Ring W and its R^{w} substituents is

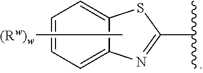

In some embodiments, Ring W and its R^{w} substituents is

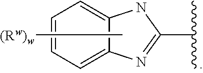

In some embodiments, Ring W and its R^{w} substituents is

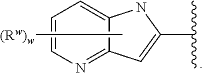

In some embodiments, Ring W and its R^{w} substituents is

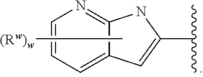

In some embodiments, Ring W and its R^{w} substituents is

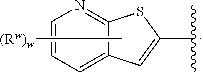

In some embodiments, Ring W and its R^{w} substituents is

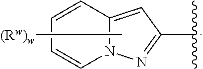

In some embodiments, Ring W and its R^{w} substituents is

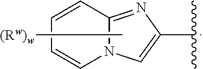

In some embodiments, Ring W and its R^{w} substituents is

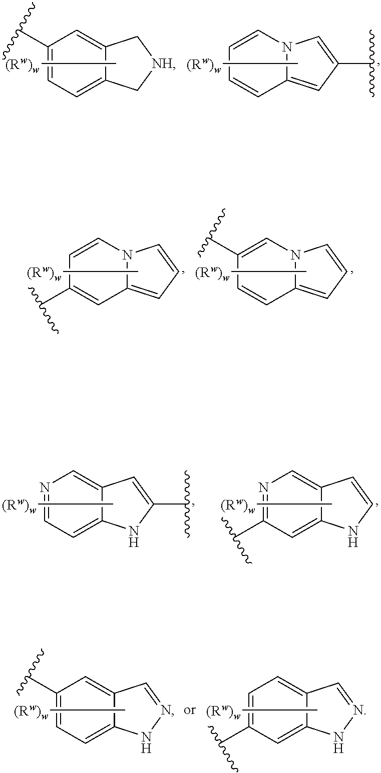

In some embodiments, Ring W and its R^{w} substituents is

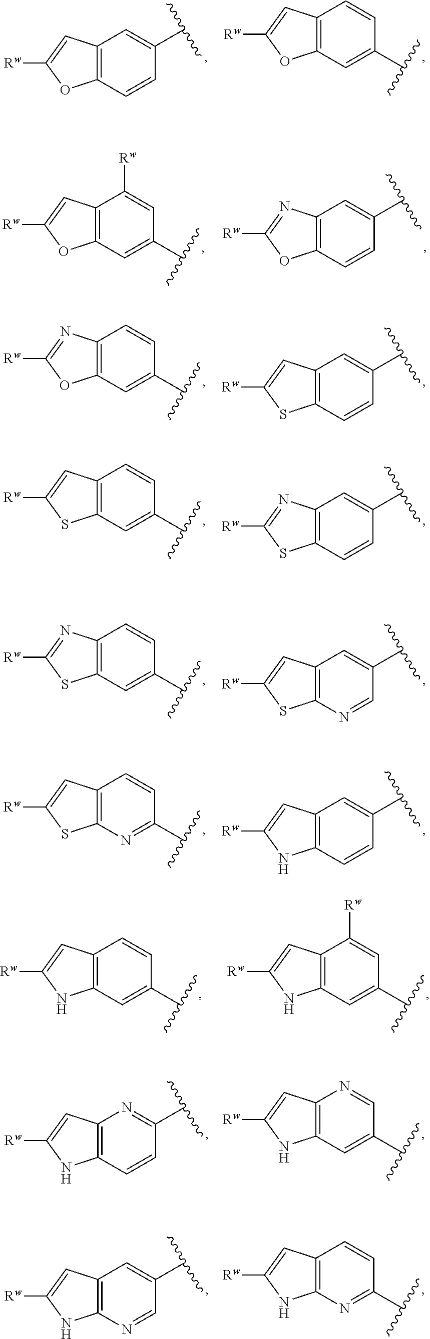

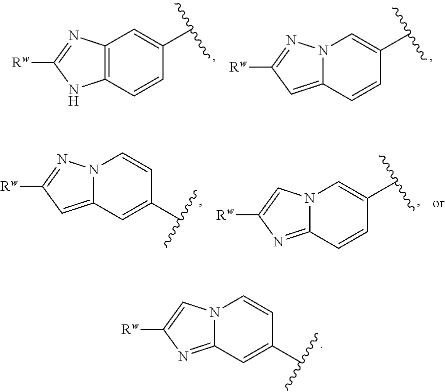

In some embodiments, Ring W and its R^{w} substituents is

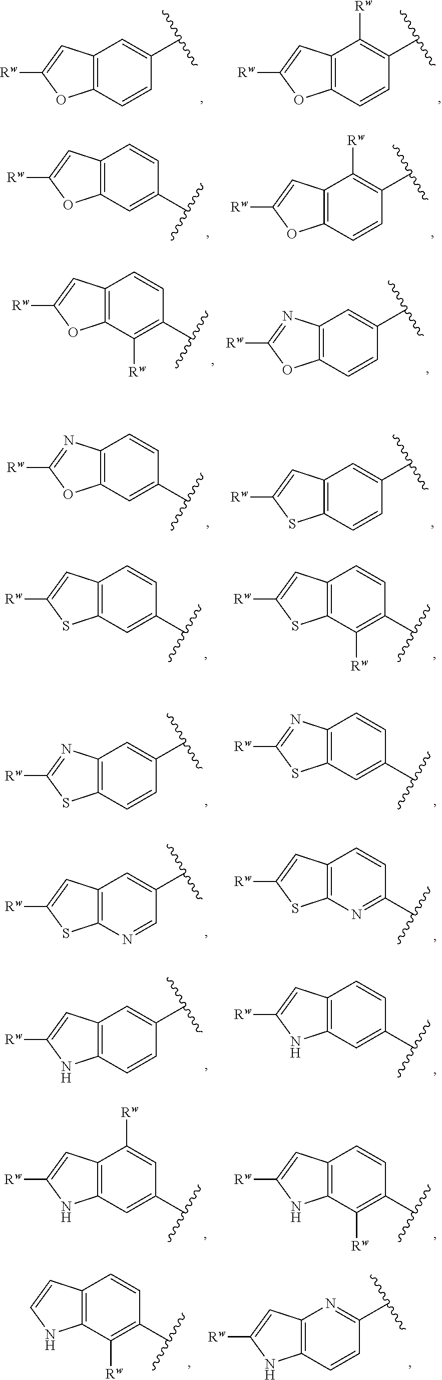

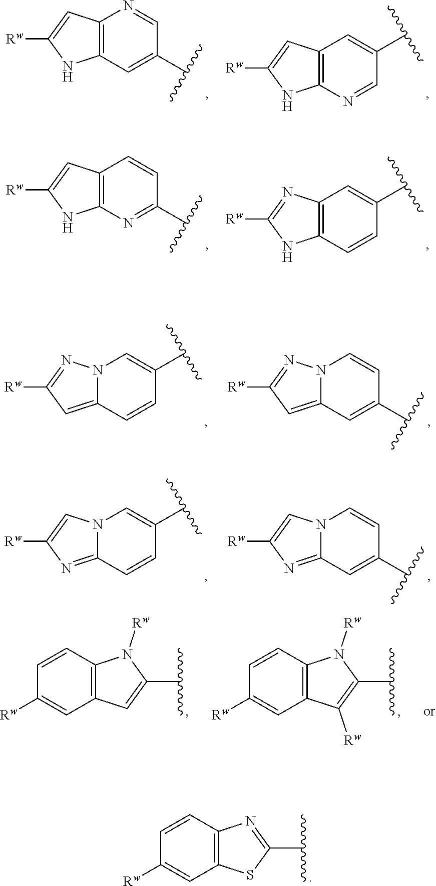

In some embodiments, Ring W and its R^{w} substituents is

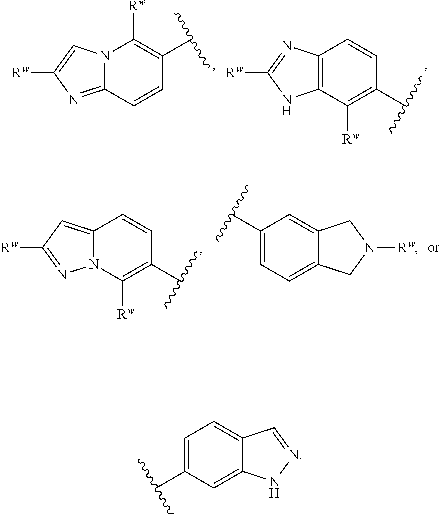

In some embodiments, Ring W and its R^{w} substituents is

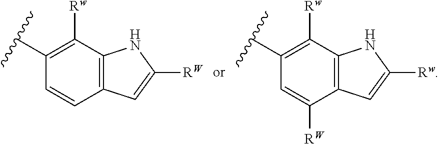

In some embodiments Ring W and its R^{w} substituents is

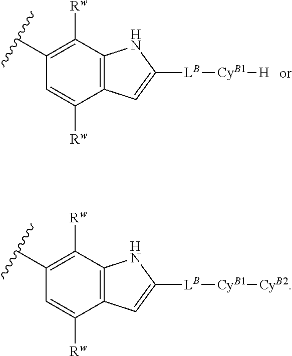

In some embodiments Ring W and its R^{w} substituents is

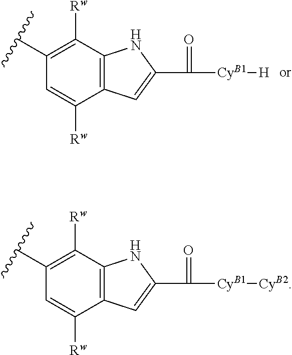

In some embodiments, Ring W and its R^{w} substituents is

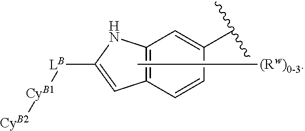

In some embodiments, Ring W and its R^{w} substituents is

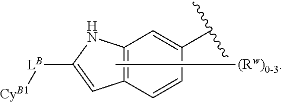

In some embodiments, Ring W and its R^{w} substituents is

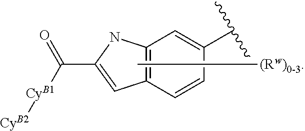

In some embodiments, Ring W and its R^{w} substituents is

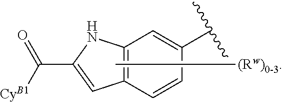

In some embodiments, Ring W and its R^{w} substituents is

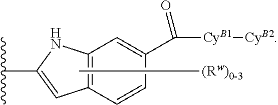

In some embodiments, Ring W and its R^{w} substituents is

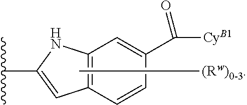

In some embodiments, Ring W and its R^{w} substituents is

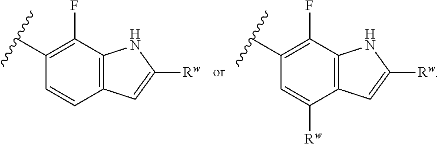

In some embodiments, Ring W and its R^{w} substituents is

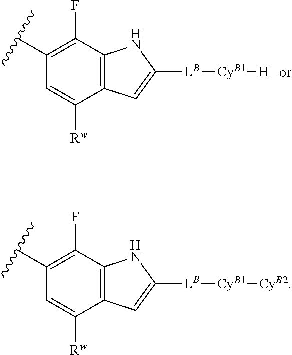

In some embodiments, Ring W and its R^{w} substituents is

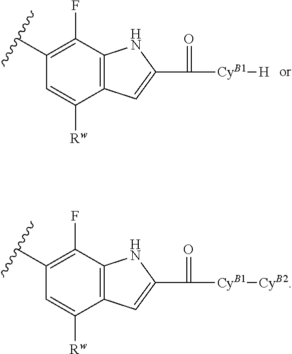

In some embodiments, Ring W and its R^{w} substituents is

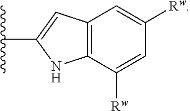

In some embodiments, Ring W and its R^{w} substituents is

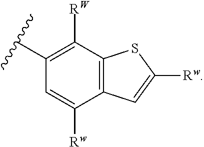

In some embodiments, Ring W and its R^{w} substituents is

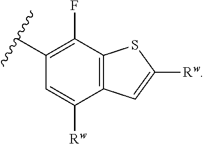

In some embodiments, Ring W and its R^{w} substituents is

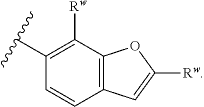

In some embodiments, Ring W and its R^{W} substituents is

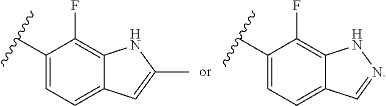 [SMILES: *c1ccc2cc(C)[nH]c2c1F ; "or" ; *c1ccc2cn[nH]c2c1F]

In some embodiment, Ring W is as depicted in the compounds of Table 1A, below.

In some embodiments, Ring W and its R^{w} substituents are

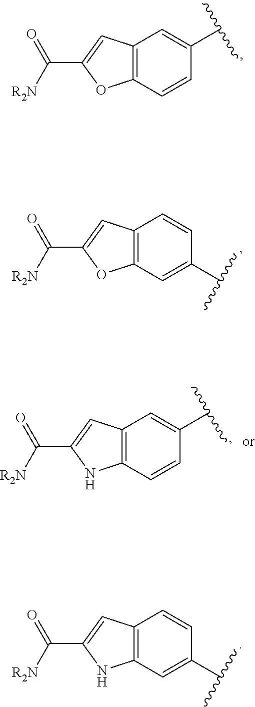

In some embodiments, Ring W and its R^{w} substituents are

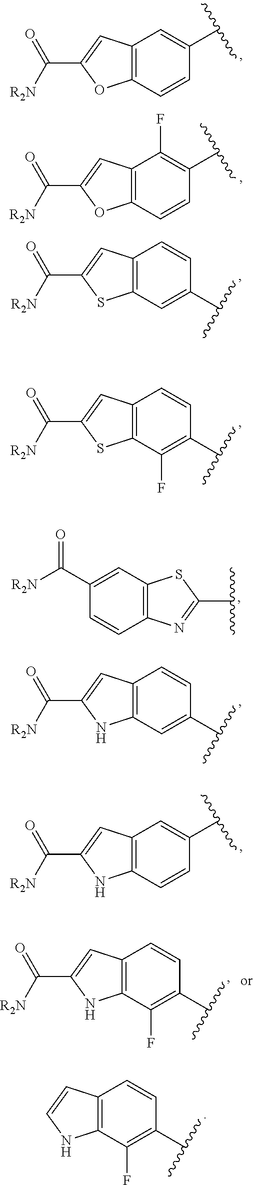

In some embodiments, Ring W and its R^{w} substituents are

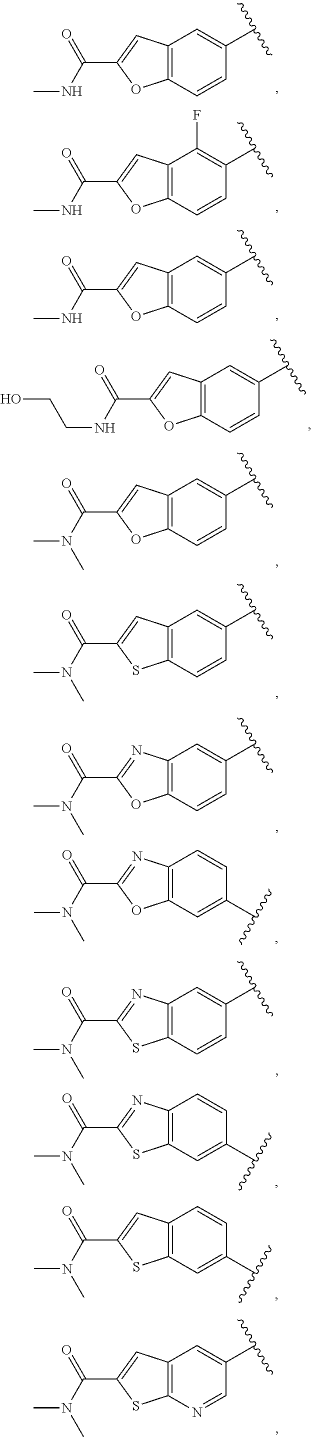

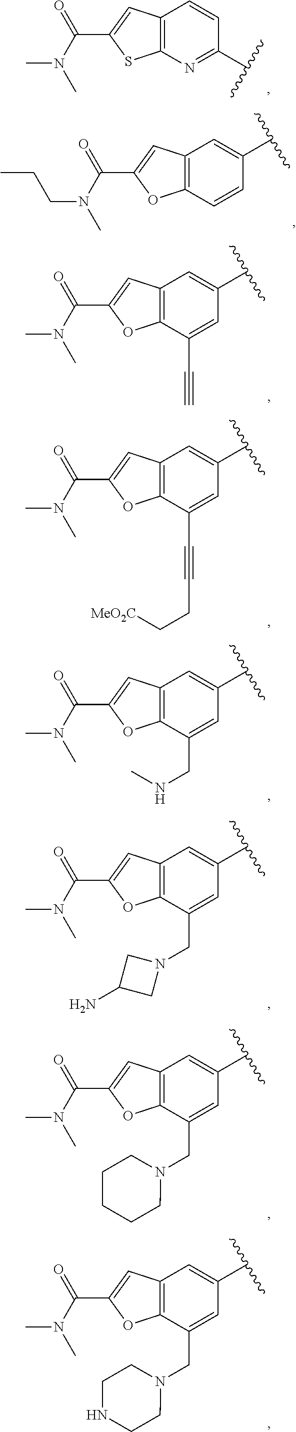

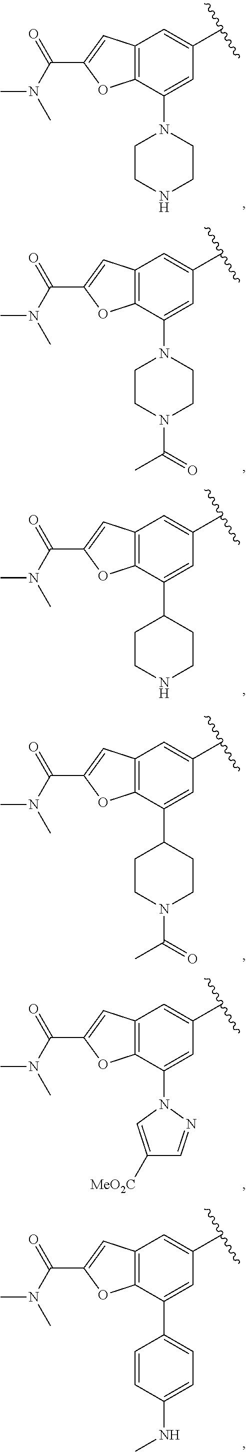 [SMILES: *c1cc(N2CCNCC2)c2oc(C(=O)N(C)C)cc2c1 ; *c1cc(N2CCN(C(C)=O)CC2)c2oc(C(=O)N(C)C)cc2c1 ; *c1cc(C2CCNCC2)c2oc(C(=O)N(C)C)cc2c1 ; *c1cc(C2CCN(C(C)=O)CC2)c2oc(C(=O)N(C)C)cc2c1 ; *c1cc(-n2cc(C(=O)OC)cn2)c2oc(C(=O)N(C)C)cc2c1 ; *c1cc(-c2ccc(NC)cc2)c2oc(C(=O)N(C)C)cc2c1]

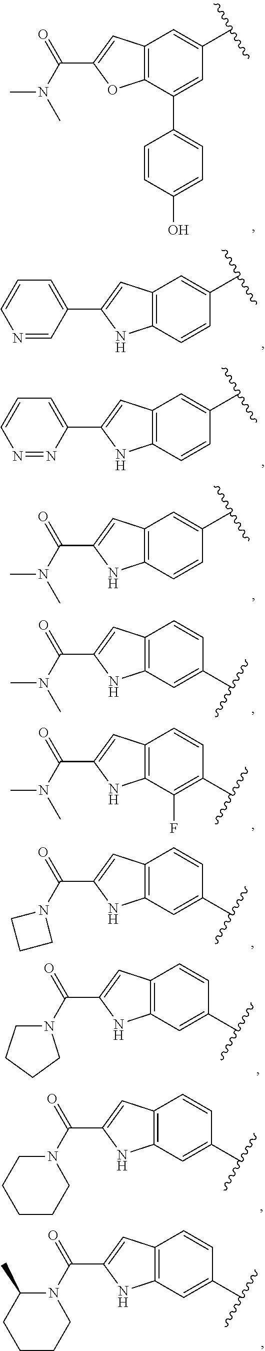

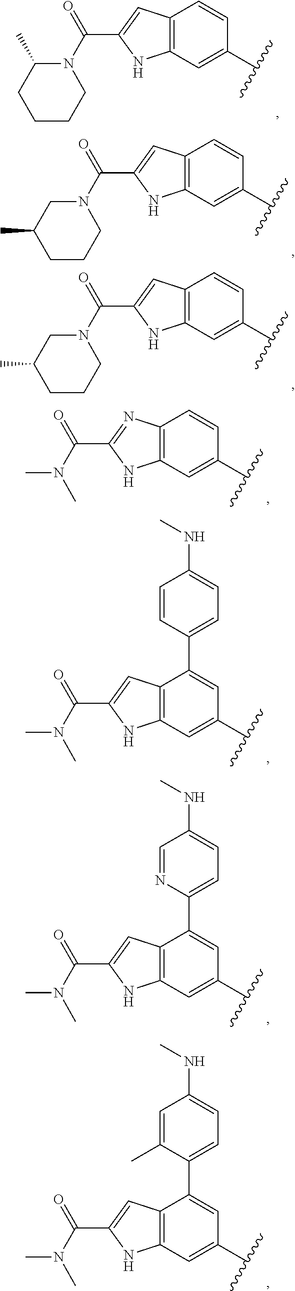

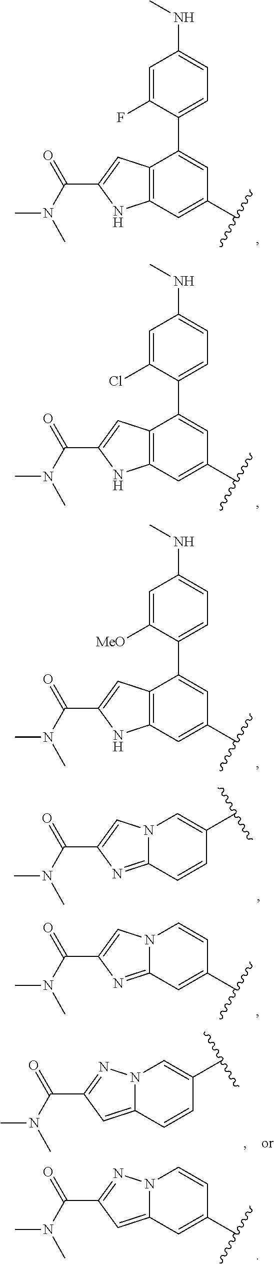

In some embodiments, Ring W and its R^{w} substituents are

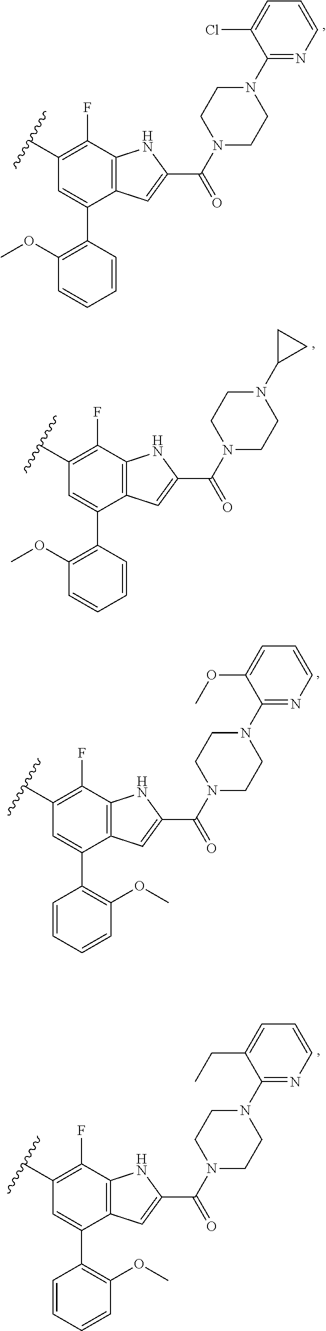 [SMILES: *c1cc(-c2ccccc2OC)c2cc(C(=O)N3CCN(c4ncccc4Cl)CC3)[nH]c2c1F ; *c1cc(-c2ccccc2OC)c2cc(C(=O)N3CCN(C4CC4)CC3)[nH]c2c1F ; *c1cc(-c2ccccc2OC)c2cc(C(=O)N3CCN(c4ncccc4OC)CC3)[nH]c2c1F ; *c1cc(-c2ccccc2OC)c2cc(C(=O)N3CCN(c4ncccc4CC)CC3)[nH]c2c1F]

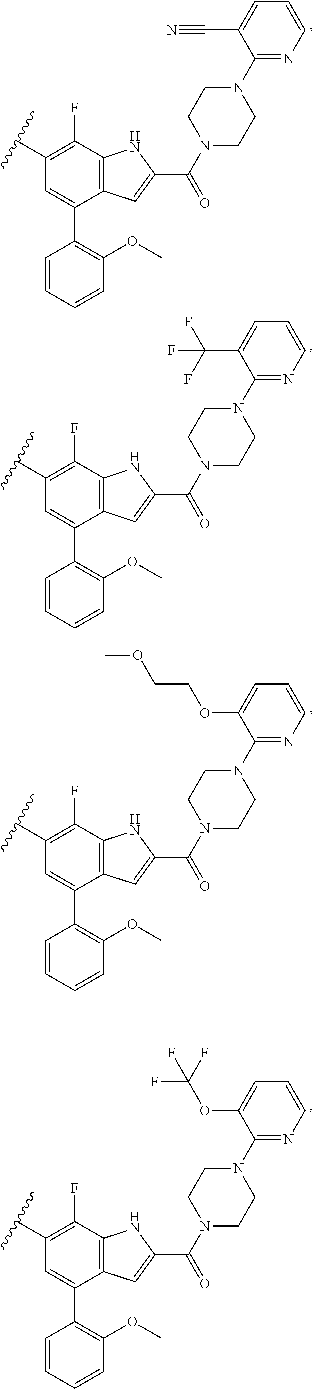

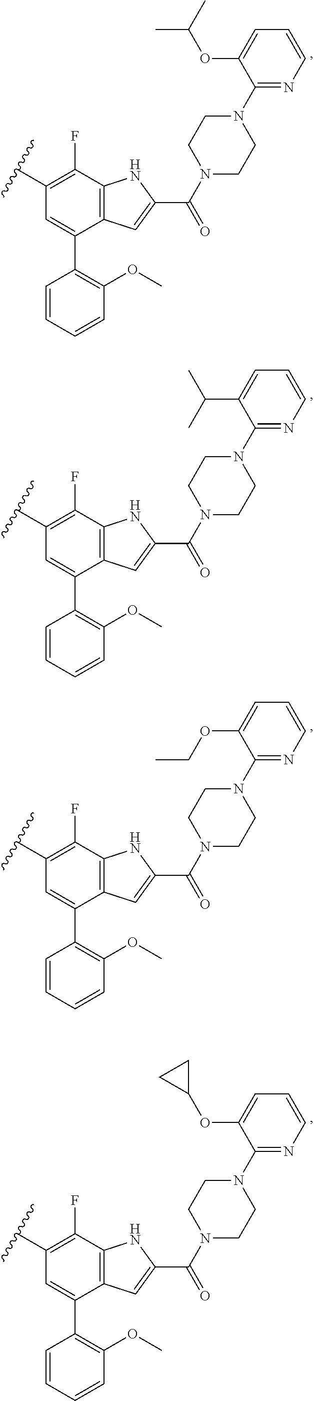 [SMILES: *c1cc(-c2ccccc2OC)c2cc(C(=O)N3CCN(c4ncccc4OC(C)C)CC3)[nH]c2c1F ; *c1cc(-c2ccccc2OC)c2cc(C(=O)N3CCN(c4ncccc4C(C)C)CC3)[nH]c2c1F ; *c1cc(-c2ccccc2OC)c2cc(C(=O)N3CCN(c4ncccc4OCC)CC3)[nH]c2c1F ; *c1cc(-c2ccccc2OC)c2cc(C(=O)N3CCN(c4ncccc4OC4CC4)CC3)[nH]c2c1F]

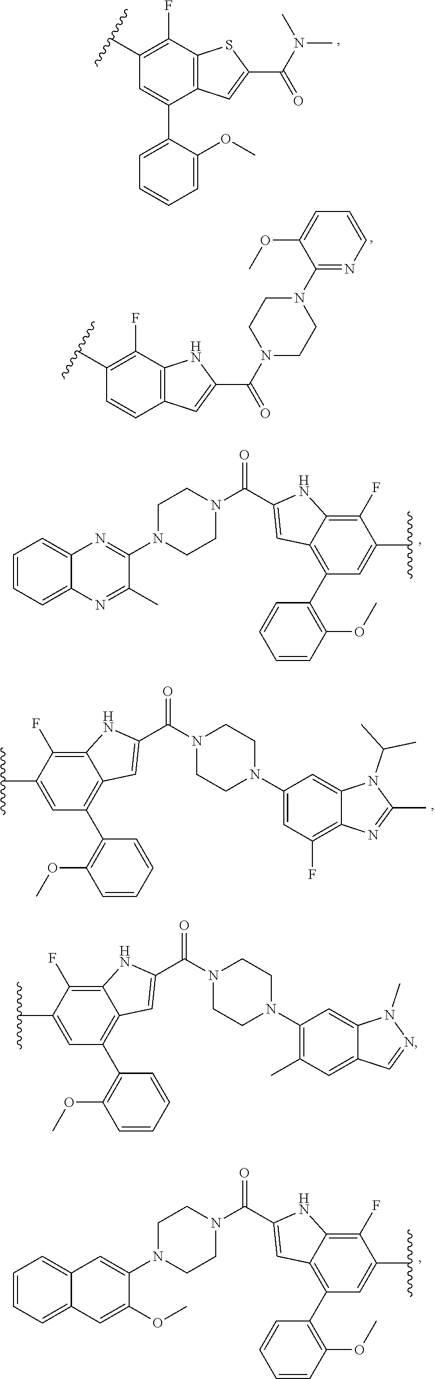 [SMILES: *c1cc(-c2ccccc2OC)c2cc(C(=O)N(C)C)sc2c1F ; *c1ccc2cc(C(=O)N3CCN(c4ncccc4OC)CC3)[nH]c2c1F ; *c1cc(-c2ccccc2OC)c2cc(C(=O)N3CCN(c4nc5ccccc5nc4C)CC3)[nH]c2c1F ; *c1cc(-c2ccccc2OC)c2cc(C(=O)N3CCN(c4cc(F)c5nc(C)n(C(C)C)c5c4)CC3)[nH]c2c1F ; *c1cc(-c2ccccc2OC)c2cc(C(=O)N3CCN(c4cc5c(cnn5C)cc4C)CC3)[nH]c2c1F ; *c1cc(-c2ccccc2OC)c2cc(C(=O)N3CCN(c4cc5ccccc5cc4OC)CC3)[nH]c2c1F]

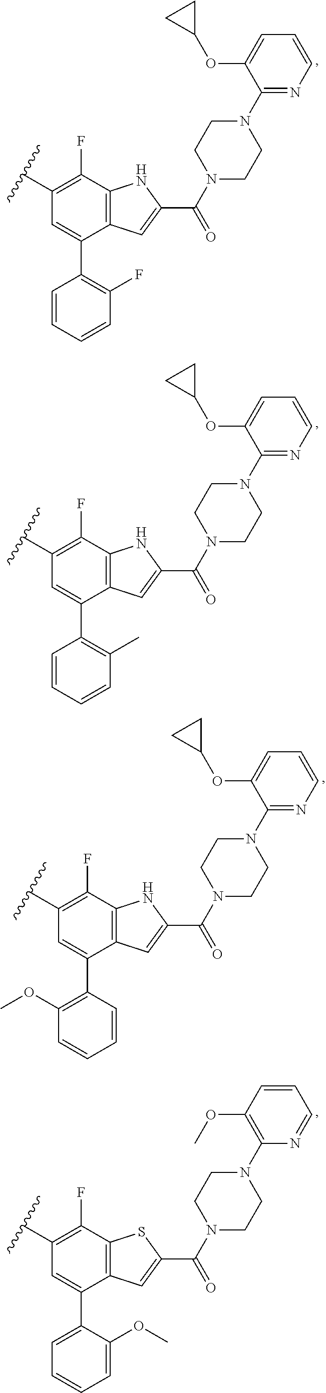 [SMILES: *c1cc(-c2ccccc2F)c2cc(C(=O)N3CCN(c4ncccc4OC4CC4)CC3)[nH]c2c1F ; *c1cc(-c2ccccc2C)c2cc(C(=O)N3CCN(c4ncccc4OC4CC4)CC3)[nH]c2c1F ; *c1cc(-c2ccccc2OC)c2cc(C(=O)N3CCN(c4ncccc4OC4CC4)CC3)[nH]c2c1F ; *c1cc(-c2ccccc2OC)c2cc(C(=O)N3CCN(c4ncccc4OC)CC3)sc2c1F]

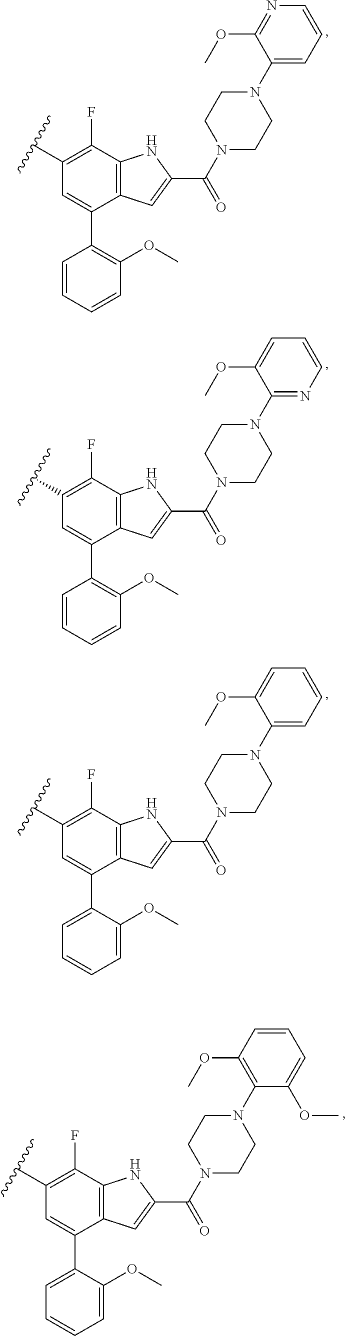

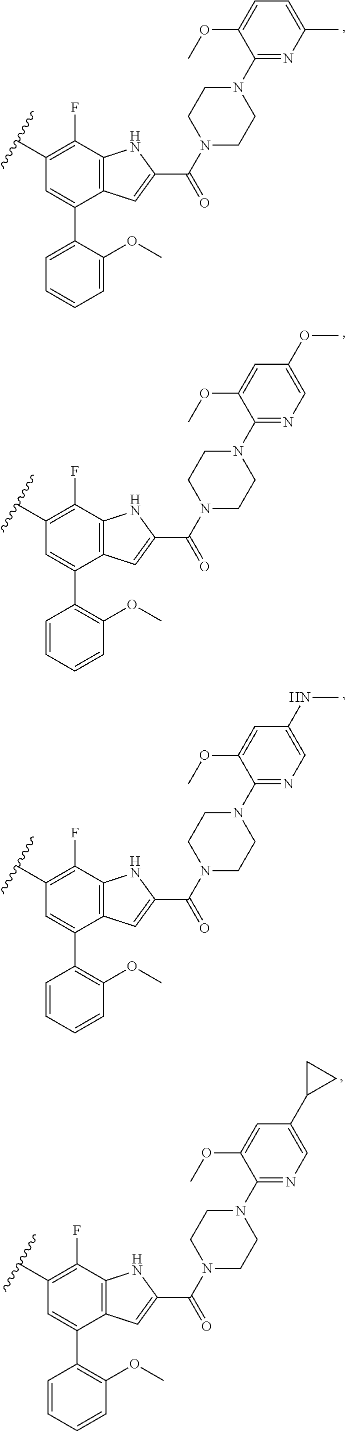 [SMILES: *c1cc(-c2ccccc2OC)c2cc(C(=O)N3CCN(c4nc(C)ccc4OC)CC3)[nH]c2c1F ; *c1cc(-c2ccccc2OC)c2cc(C(=O)N3CCN(c4ncc(OC)cc4OC)CC3)[nH]c2c1F ; *c1cc(-c2ccccc2OC)c2cc(C(=O)N3CCN(c4ncc(NC)cc4OC)CC3)[nH]c2c1F ; *c1cc(-c2ccccc2OC)c2cc(C(=O)N3CCN(c4ncc(C5CC5)cc4OC)CC3)[nH]c2c1F]

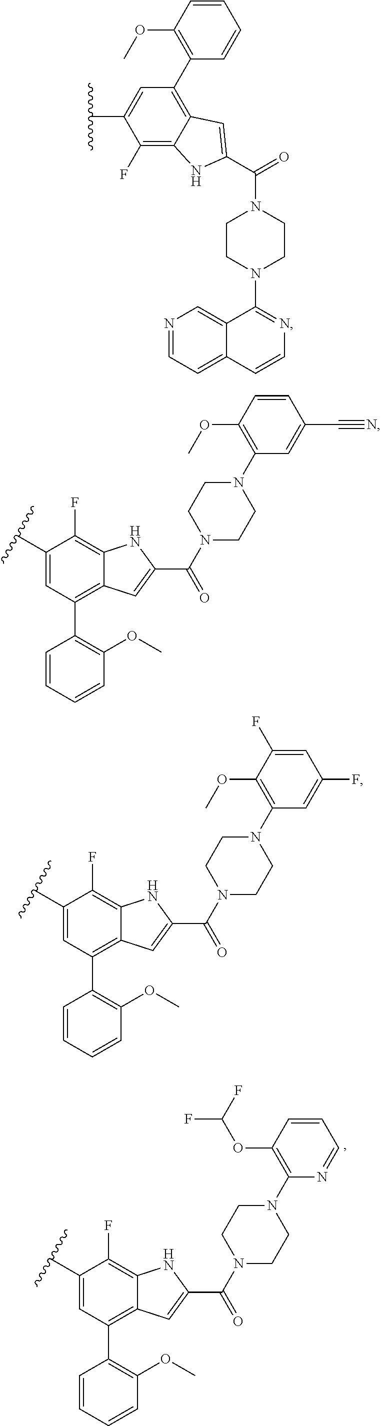

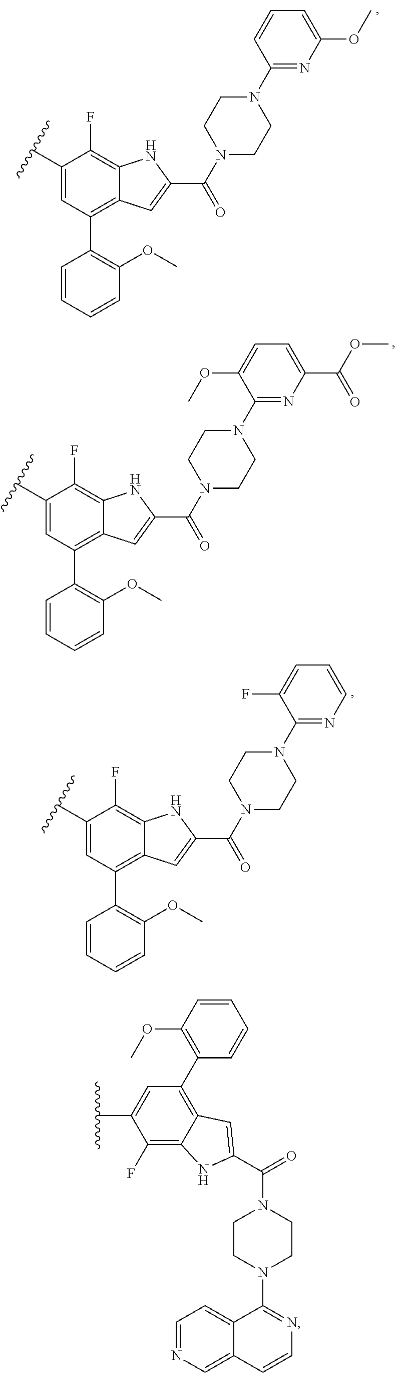 [SMILES: *c1cc(-c2ccccc2OC)c2cc(C(=O)N3CCN(c4cccc(OC)n4)CC3)[nH]c2c1F ; *c1cc(-c2ccccc2OC)c2cc(C(=O)N3CCN(c4nc(C(=O)OC)ccc4OC)CC3)[nH]c2c1F ; *c1cc(-c2ccccc2OC)c2cc(C(=O)N3CCN(c4ncccc4F)CC3)[nH]c2c1F ; *c1cc(-c2ccccc2OC)c2cc(C(=O)N3CCN(c4nccc5cnccc45)CC3)[nH]c2c1F]

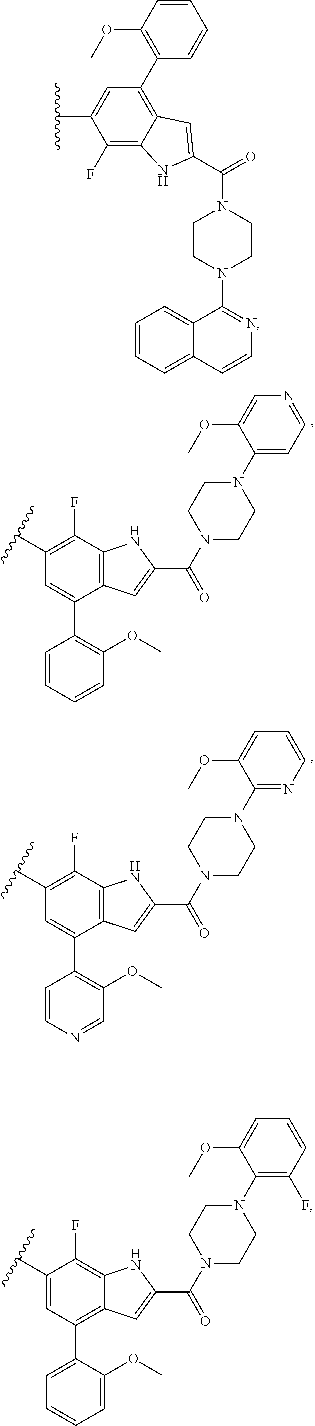

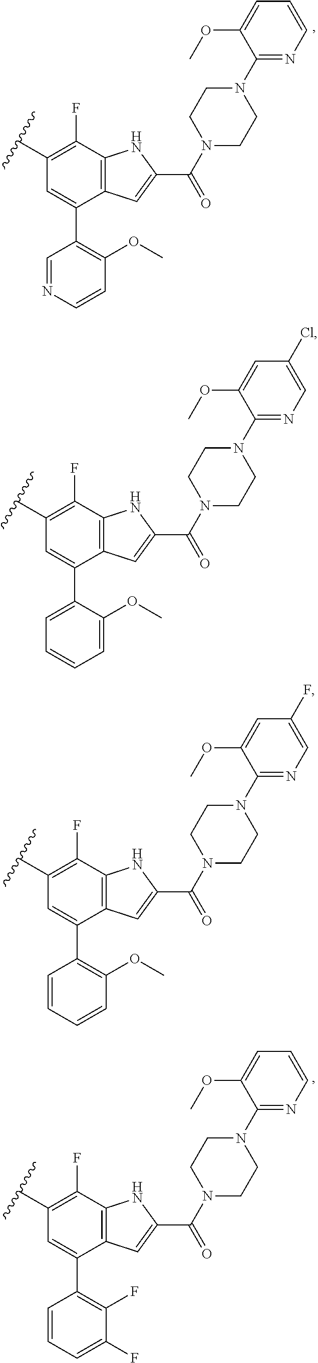 [SMILES: *c1cc(-c2cnccc2OC)c2cc(C(=O)N3CCN(c4ncccc4OC)CC3)[nH]c2c1F ; *c1cc(-c2ccccc2OC)c2cc(C(=O)N3CCN(c4ncc(Cl)cc4OC)CC3)[nH]c2c1F ; *c1cc(-c2ccccc2OC)c2cc(C(=O)N3CCN(c4ncc(F)cc4OC)CC3)[nH]c2c1F ; *c1cc(-c2cccc(F)c2F)c2cc(C(=O)N3CCN(c4ncccc4OC)CC3)[nH]c2c1F]

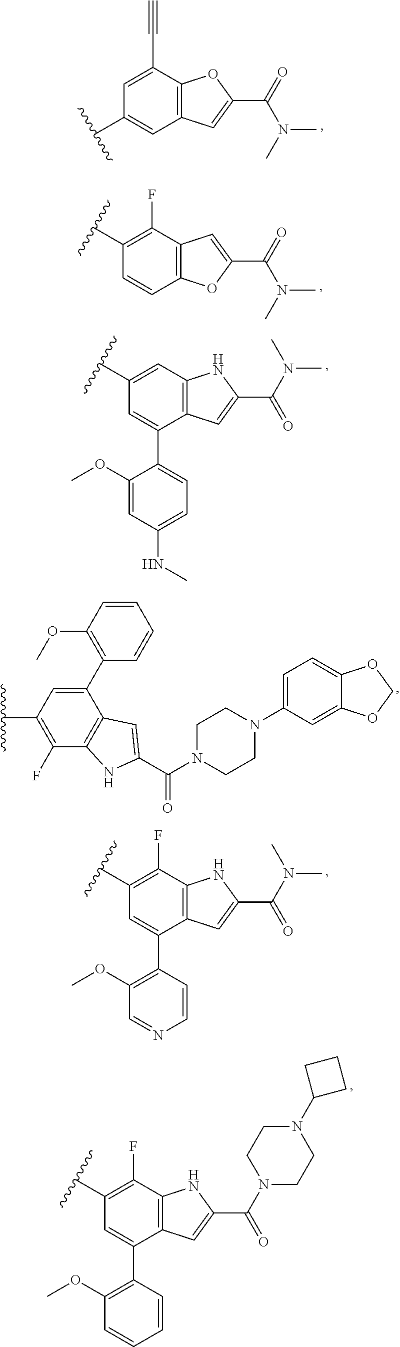

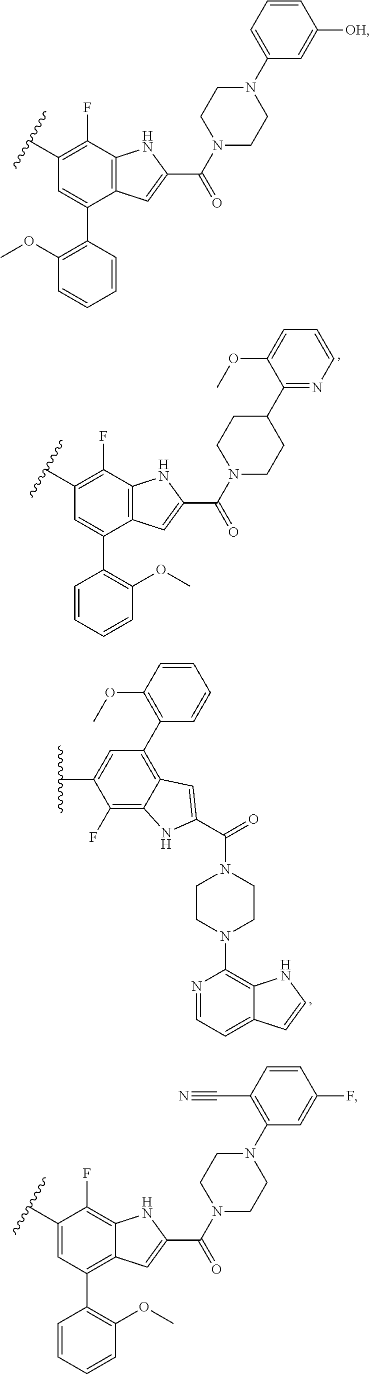

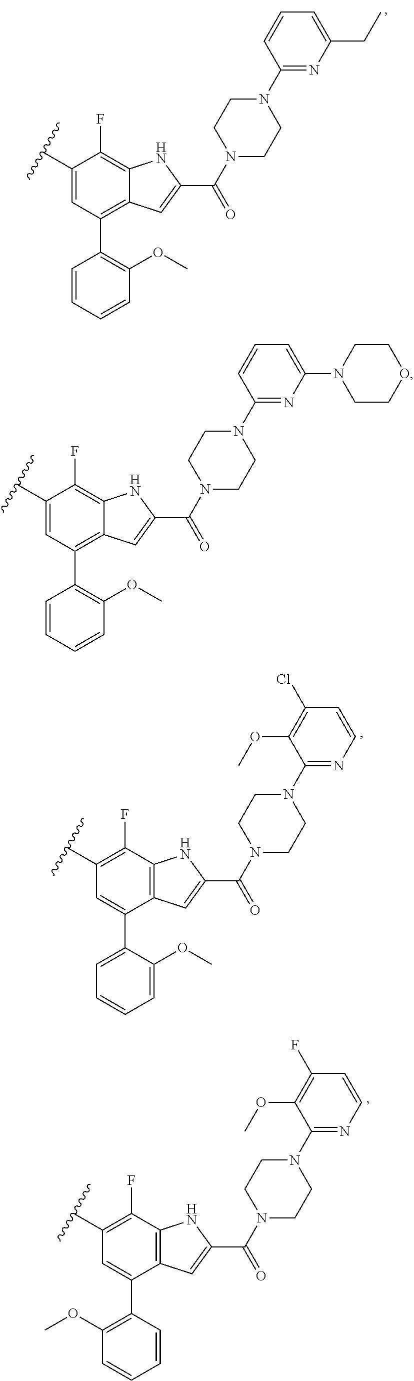

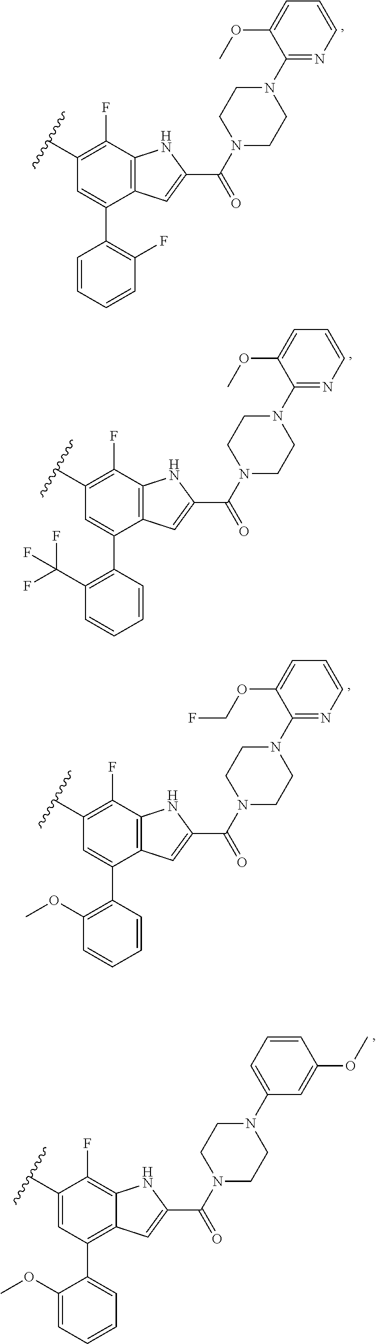

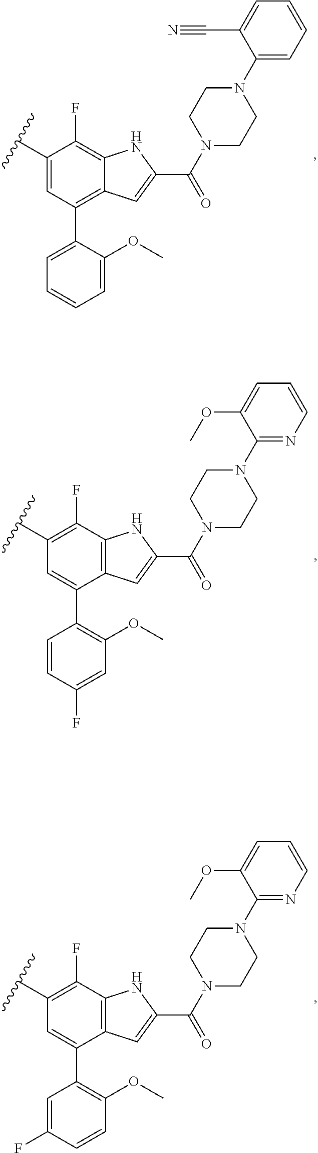 [SMILES: *c1cc(-c2ccccc2OC)c2cc(C(=O)N3CCN(c4ccccc4C#N)CC3)[nH]c2c1F ; *c1cc(-c2ccc(F)cc2OC)c2cc(C(=O)N3CCN(c4ncccc4OC)CC3)[nH]c2c1F ; *c1cc(-c2cc(F)ccc2OC)c2cc(C(=O)N3CCN(c4ncccc4OC)CC3)[nH]c2c1F]

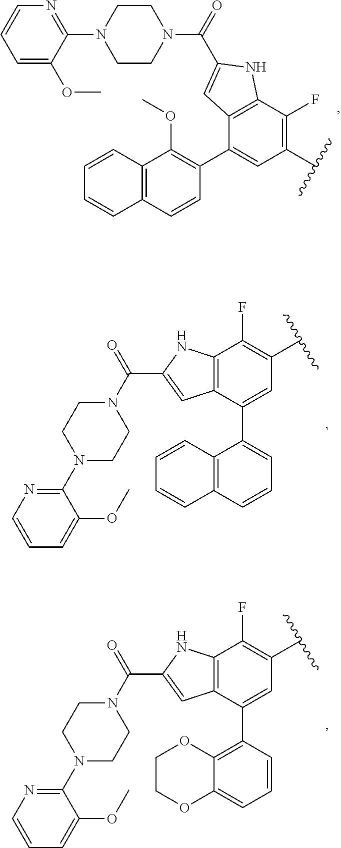 [SMILES: *c1cc(-c2ccc3ccccc3c2OC)c2cc(C(=O)N3CCN(c4ncccc4OC)CC3)[nH]c2c1F ; *c1cc(-c2cccc3ccccc23)c2cc(C(=O)N3CCN(c4ncccc4OC)CC3)[nH]c2c1F ; *c1cc(-c2cccc3c2OCCO3)c2cc(C(=O)N3CCN(c4ncccc4OC)CC3)[nH]c2c1F]

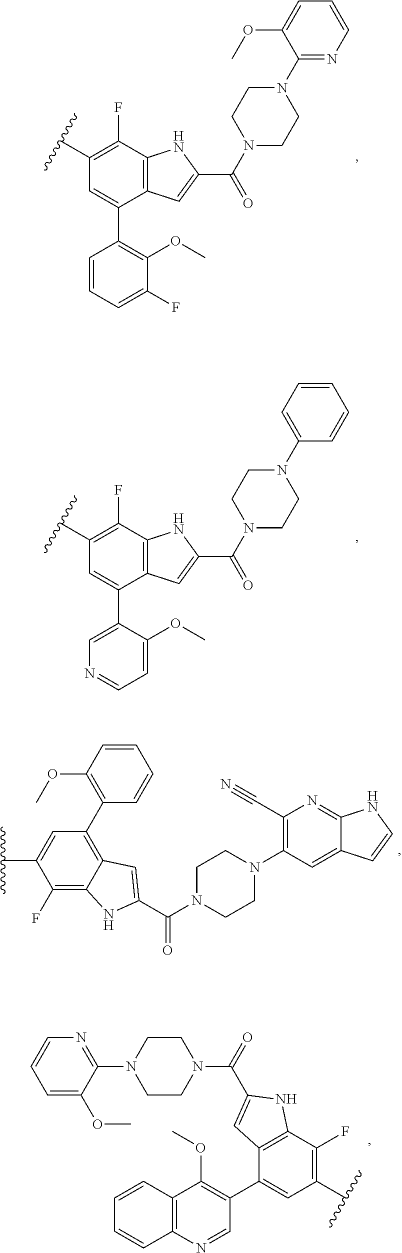 [SMILES: *c1cc(-c2cccc(F)c2OC)c2cc(C(=O)N3CCN(c4ncccc4OC)CC3)[nH]c2c1F ; *c1cc(-c2cnccc2OC)c2cc(C(=O)N3CCN(c4ccccc4)CC3)[nH]c2c1F ; *c1cc(-c2ccccc2OC)c2cc(C(=O)N3CCN(c4cc5cc[nH]c5nc4C#N)CC3)[nH]c2c1F ; *c1cc(-c2cnc3ccccc3c2OC)c2cc(C(=O)N3CCN(c4ncccc4OC)CC3)[nH]c2c1F]

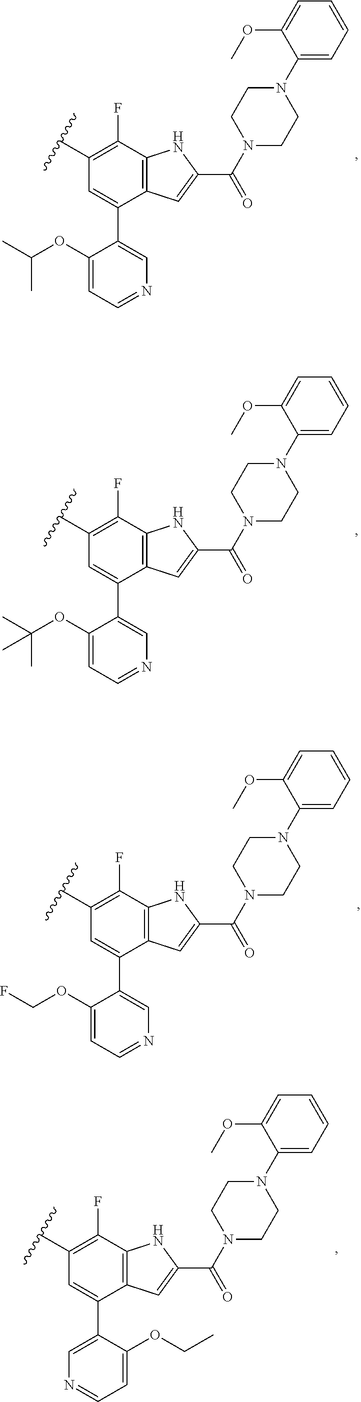 [SMILES: *c1cc(-c2cnccc2OC(C)C)c2cc(C(=O)N3CCN(c4ccccc4OC)CC3)[nH]c2c1F ; *c1cc(-c2cnccc2OC(C)(C)C)c2cc(C(=O)N3CCN(c4ccccc4OC)CC3)[nH]c2c1F ; *c1cc(-c2cnccc2OCF)c2cc(C(=O)N3CCN(c4ccccc4OC)CC3)[nH]c2c1F ; *c1cc(-c2cnccc2OCC)c2cc(C(=O)N3CCN(c4ccccc4OC)CC3)[nH]c2c1F]

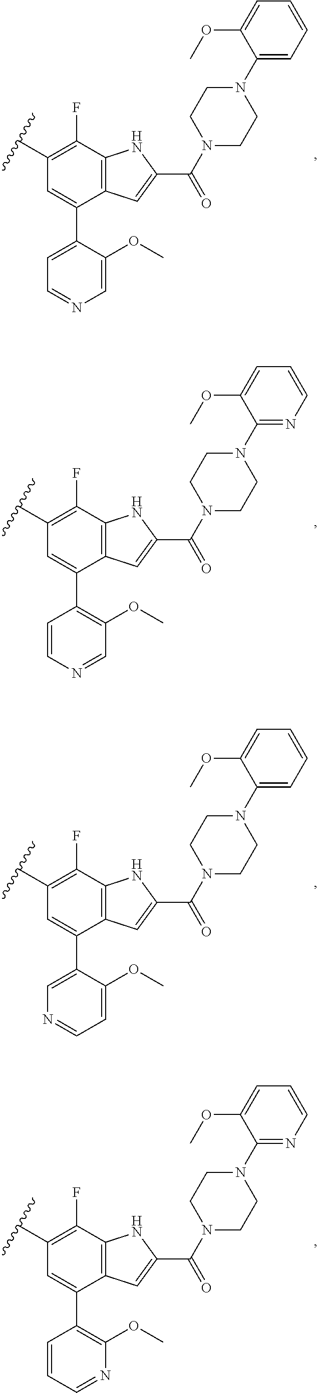 [SMILES: *c1cc(-c2ccncc2OC)c2cc(C(=O)N3CCN(c4ccccc4OC)CC3)[nH]c2c1F ; *c1cc(-c2ccncc2OC)c2cc(C(=O)N3CCN(c4ncccc4OC)CC3)[nH]c2c1F ; *c1cc(-c2cnccc2OC)c2cc(C(=O)N3CCN(c4ccccc4OC)CC3)[nH]c2c1F ; *c1cc(-c2cccnc2OC)c2cc(C(=O)N3CCN(c4ncccc4OC)CC3)[nH]c2c1F]

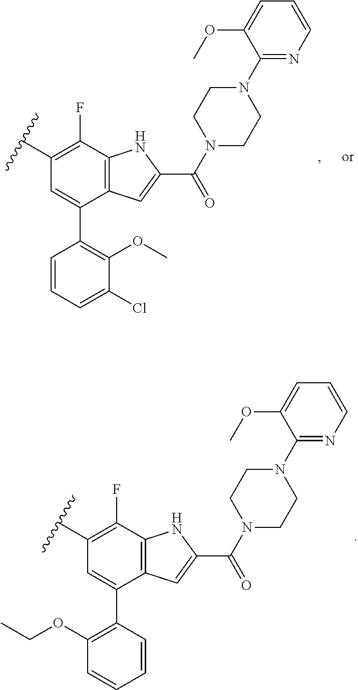 [SMILES: *c1cc(-c2cccc(Cl)c2OC)c2cc(C(=O)N3CCN(c4ncccc4OC)CC3)[nH]c2c1F ; *c1cc(-c2ccccc2OCC)c2cc(C(=O)N3CCN(c4ncccc4OC)CC3)[nH]c2c1F]

In some embodiments, Ring W and its R^{w} substituents are

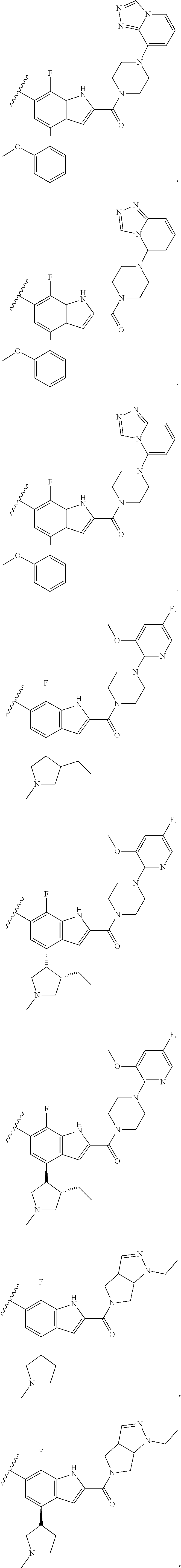

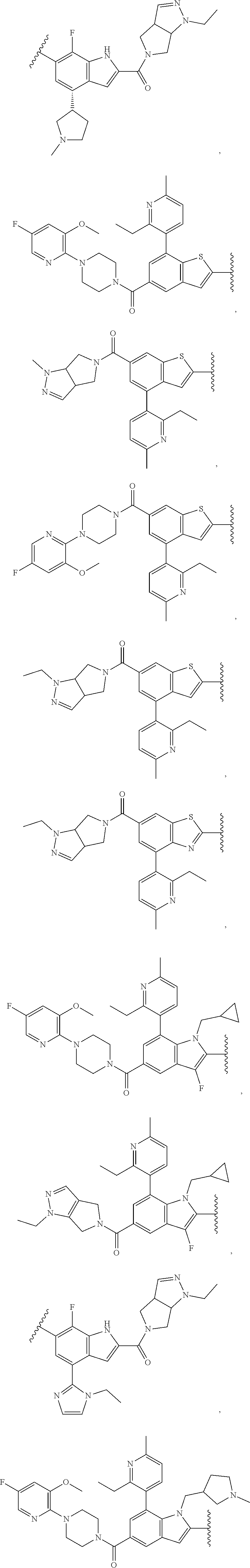

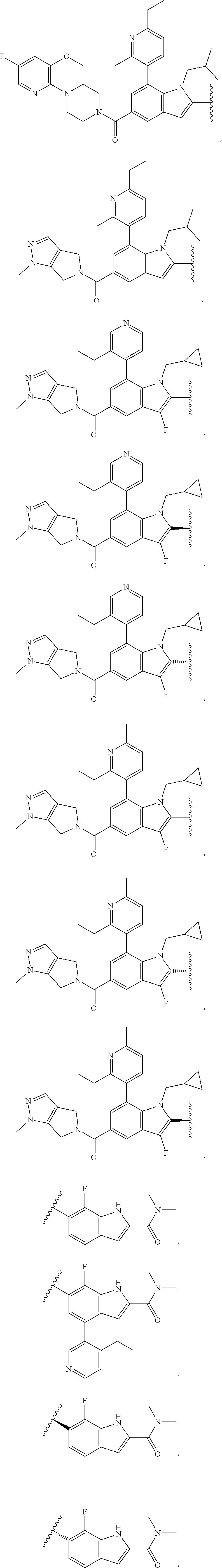

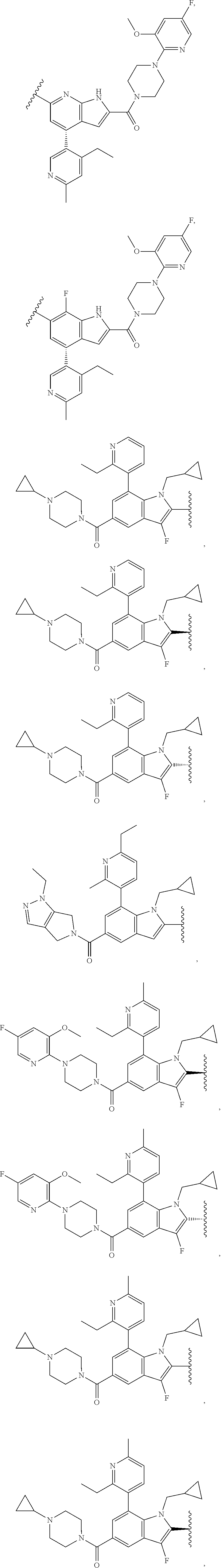

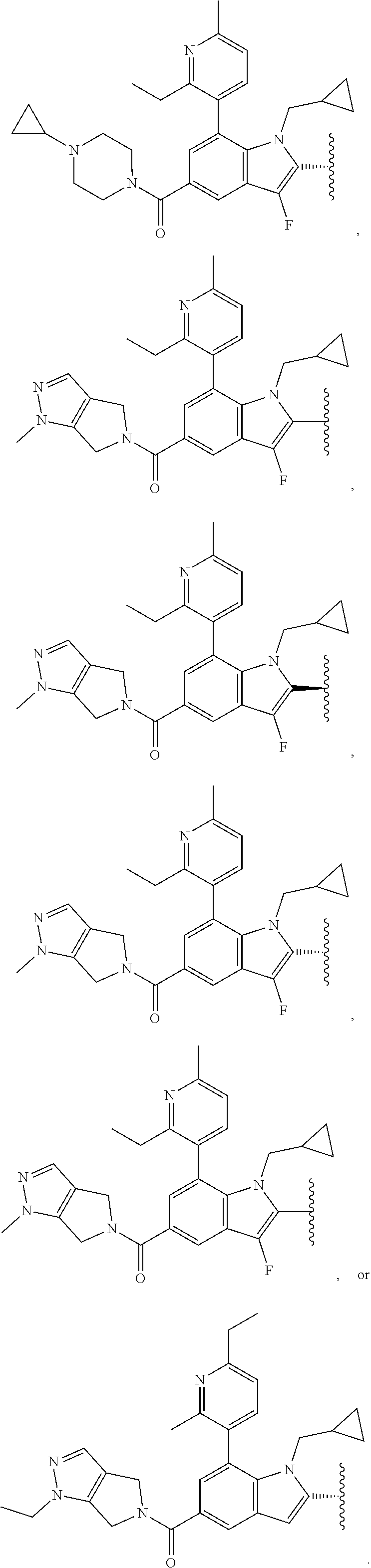

As described above and defined herein, Ring X and its R^{x} substituents is

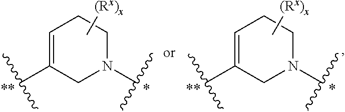

wherein

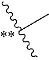

represents a bond to Ring W, and

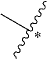

represents a bond to L^{x} or to the rest of the molecule.

In some embodiments, Ring X and its R^{x} substituents is

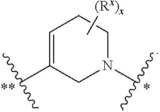

In some embodiments, Ring X and its R^{x} substituents is

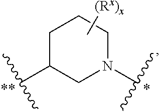

In some embodiments, Ring X and its R^{x} substituents is

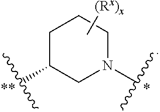

In some embodiments, Ring X and its R^{x} substituents is

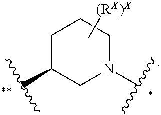

In some embodiment, Ring X is as depicted in the compounds of Table 1A, below.

In some embodiments, Ring X and its R^{x} substituents are

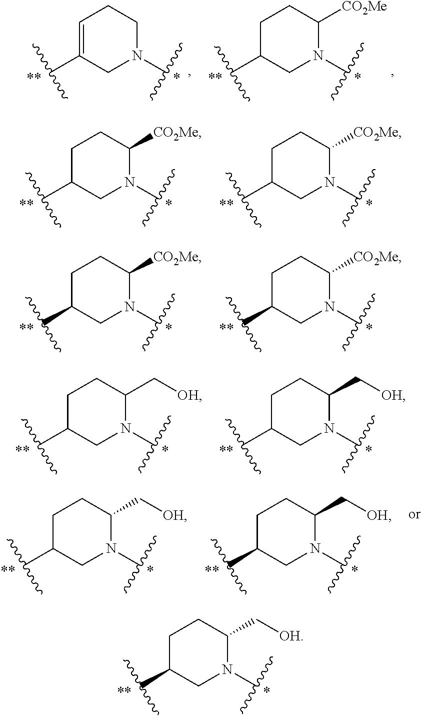

In some embodiments, Ring X and its R^{x} substituents are

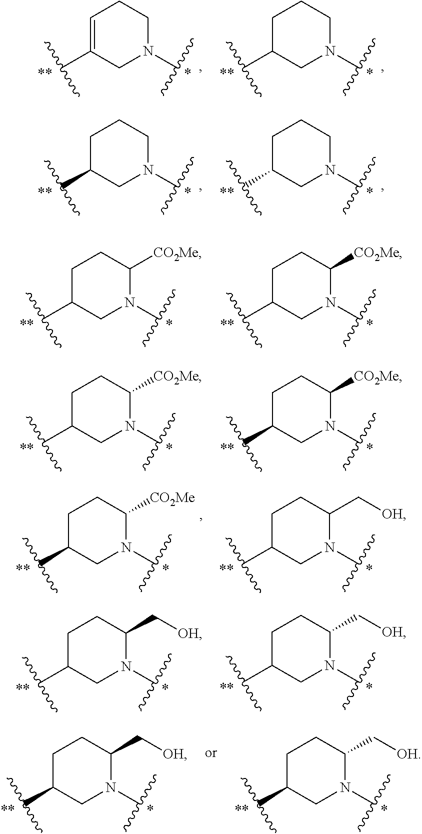

In some embodiments, Ring X and its R^{x} substituents are

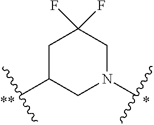 [SMILES: *C1CN(*)CC(F)(F)C1]

As described above and defined herein, Ring Y is a ring selected from phenyl, naphthyl, 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-10 membered monocyclic or bicyclic heteroaryl with 1-4 heteroatoms independently selected from nitrogen, oxygen and sulfur.

In some embodiments, Ring Y is a ring selected from phenyl, 3-8 membered saturated or partially unsaturated monocyclic carbocyclyl or heterocyclyl with 1-3 heteroatoms independently selected from nitrogen, oxygen, and sulfur, and 5-6 membered monocyclic heteroaryl with 1-4 heteroatoms independently selected from nitrogen, oxygen and sulfur.

In some embodiments, Ring Y is a ring selected from naphthyl, 5-10 membered saturated or partially unsaturated bicyclic or bridged bicyclic carbocyclyl or heterocyclyl with 1-3 heteroatoms independently selected from nitrogen, oxygen, and sulfur, and 8-10 membered bicyclic heteroaryl with 1-4 heteroatoms independently selected from nitrogen, oxygen and sulfur.

In some embodiments, Ring Y is a ring selected from phenyl, 3-8 membered saturated or partially unsaturated monocyclic carbocyclyl or heterocyclyl with 1-3 heteroatoms independently selected from nitrogen, oxygen, and sulfur, 5-6 membered monocyclic heteroaryl with 1-4 heteroatoms independently selected from nitrogen, oxygen and sulfur, naphthyl, 5-10 membered saturated or partially unsaturated bicyclic or bridged bicyclic carbocyclyl or heterocyclyl with 1-3 heteroatoms independently selected from nitrogen, oxygen, and sulfur, and 8-10 membered bicyclic heteroaryl with 1-4 heteroatoms independently selected from nitrogen, oxygen and sulfur.

In some embodiments, Ring Y is phenyl. In some embodiments, Ring Y is naphthyl. In some embodiments, Ring Y is a 3-11 membered saturated or partially unsaturated monocyclic, bicyclic, bridged bicyclic, or spirocyclic carbocyclyl. In some embodiments, Ring Y is a 5-11 membered saturated or partially unsaturated monocyclic, bicyclic, bridged bicyclic, or spirocyclic heterocyclyl with 1-3 heteroatoms independently selected from nitrogen, oxygen, and sulfur. In some embodiments, Ring Y is a 5-10 membered monocyclic or bicyclic heteroaryl with 1-4 heteroatoms independently selected from nitrogen, oxygen, and sulfur. In some embodiments, Ring Y is a 5-10 membered monocyclic or bicyclic heteroaryl with 1-4 heteroatoms independently selected from nitrogen, oxygen, and sulfur.

In some embodiments, Ring Y is a 3-8 membered saturated or partially unsaturated monocyclic carbocyclyl. In some embodiments, Ring Y is a 3-6 membered saturated or partially unsaturated monocyclic carbocyclyl. In some embodiments, Ring Y is cyclopropyl. In some embodiments, Ring Y is cyclobutyl. In some embodiments, Ring Y is cyclopentyl. In some embodiments, Ring Y is hexyl.

In some embodiments, Ring Y is a 5-6 membered saturated or partially unsaturated monocyclic carbocyclyl. In some embodiments, Ring Y is a 6-7 membered saturated or partially unsaturated monocyclic carbocyclyl. In some embodiments, Ring Y is a 3 membered saturated or partially unsaturated monocyclic carbocyclyl. In some embodiments, Ring Y is a 4 membered saturated or partially unsaturated monocyclic carbocyclyl. In some embodiments, Ring Y is a 5 membered saturated or partially unsaturated monocyclic carbocyclyl. In some embodiments, Ring Y is a 6 membered saturated or partially unsaturated monocyclic carbocyclyl. In some embodiments, Ring Y is a 7 membered saturated or partially unsaturated monocyclic carbocyclyl. In some embodiments, Ring Y is cyclopropyl, cyclobutyl, cyclopentyl, cyclohexyl, or cycloheptyl.

In some embodiments, Ring Y is a 5-10 membered saturated or partially unsaturated bicyclic or bridged bicyclic carbocyclyl.

In some embodiments, Ring Y is a 5-6 membered monocyclic heteroaryl with 1-4 heteroatoms independently selected from nitrogen, oxygen and sulfur. In some embodiments, Ring Y is a 9-membered bicyclic heteroaryl with 1-4 heteroatoms independently selected from nitrogen, oxygen, and sulfur.

In some embodiments, Ring Y is a 5-membered monocyclic heteroaryl ring with 1 heteroatom selected from nitrogen, oxygen, and sulfur. In some embodiments, Ring Y is a 5-membered monocyclic heteroaryl ring with 2 heteroatoms independently selected from nitrogen, oxygen, and sulfur. In some embodiments, Ring Y is a 5-membered monocyclic heteroaryl ring with 3 heteroatoms independently selected from nitrogen, oxygen, and sulfur. In some embodiments, Ring Y is a 5-membered monocyclic heteroaryl ring with 4 heteroatoms independently selected from nitrogen, oxygen, and sulfur. In some embodiments, Ring Y is a 5-membered monocyclic heteroaryl ring with 2-3 nitrogen heteroatoms.

In some embodiments, Ring Y is a 6-membered monocyclic heteroaryl with 1-4 heteroatoms independently selected from nitrogen, oxygen, and sulfur. In some embodiments, Ring Y is a 6-membered monocyclic heteroaryl with 1-4 nitrogen heteroatoms. In some embodiments, Ring Y is pyridinyl, pyrimidinyl, pyridazinyl, or triazinyl.

In some embodiments, Ring Y is a 3-8 membered saturated or partially unsaturated monocyclic heterocyclyl with 1-3 heteroatoms independently selected from nitrogen, oxygen, and sulfur.

In some embodiments, Ring Y is an 8-11 membered saturated or partially unsaturated bicyclic, bridged bicyclic, or spirocyclic heterocyclyl with 1-3 heteroatoms independently selected from nitrogen, oxygen, and sulfur. In some embodiments, Ring Y is a 5-10 membered saturated or partially unsaturated bicyclic or bridged bicyclic heterocyclyl with 1-3 heteroatoms independently selected from nitrogen, oxygen, and sulfur. In some embodiments, Ring Y is a 8-11 membered saturated or partially unsaturated bicyclic heterocyclyl with 1-3 heteroatoms independently selected from nitrogen, oxygen, and sulfur. In some embodiments, Ring Y is a 9-10 membered saturated or partially unsaturated bicyclic heterocyclyl with 1-3 heteroatoms independently selected form nitrogen, oxygen, and sulfur. In some embodiments, Ring Y is a 9 membered saturated or partially unsaturated bicyclic heterocyclyl with 1-3 heteroatoms independently selected from nitrogen, oxygen, and sulfur. In some embodiments, Ring Y is a 10 membered saturated or partially unsaturated bicyclic heterocyclyl with 1-3 heteroatoms independently selected from nitrogen, oxygen, and sulfur. In some embodiments, Ring Y is tetrahydroisoquinolinyl or tetrahydroquinolinyl.

In some embodiments, Ring Y is a 8-10 membered bicyclic heteroaryl with 1-4 heteroatoms independently selected from nitrogen, oxygen and sulfur. In some embodiments, Ring Y is a 9-10 membered bicyclic heteroaryl with 1-4 heteroatoms independently selected from nitrogen, oxygen, and sulfur. In some embodiments, Ring Y is a 9 membered bicyclic heteroaryl with 1-4 heteroatoms independently selected from nitrogen, oxygen, and sulfur. In some embodiments, Ring Y is a 9 membered bicyclic heteroaryl with 1-4 nitrogen heteroatoms.

In some embodiments, Ring Y is

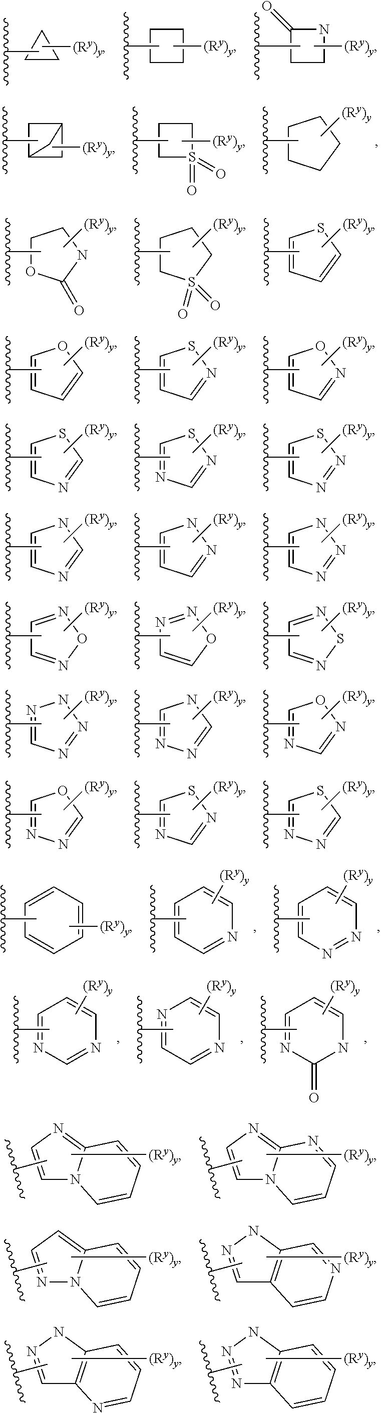

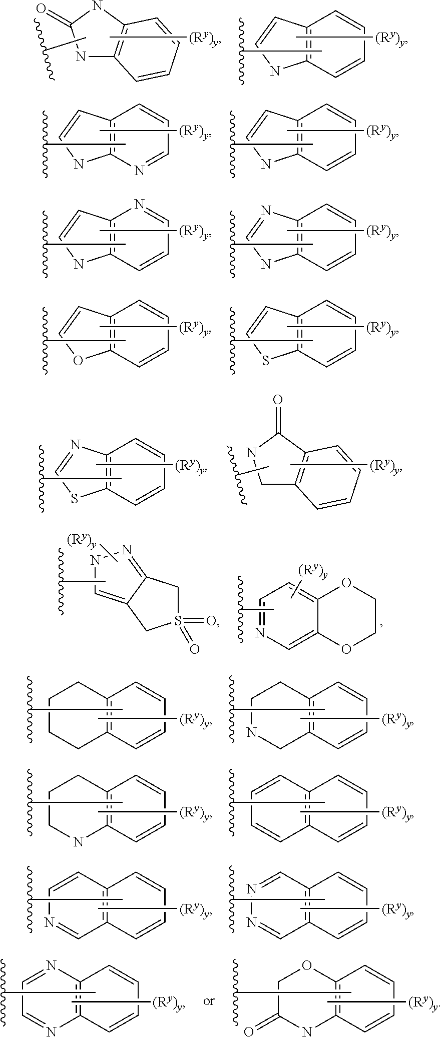

In some embodiments, Ring Y is

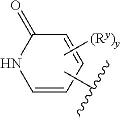

In some embodiments, Ring Y is

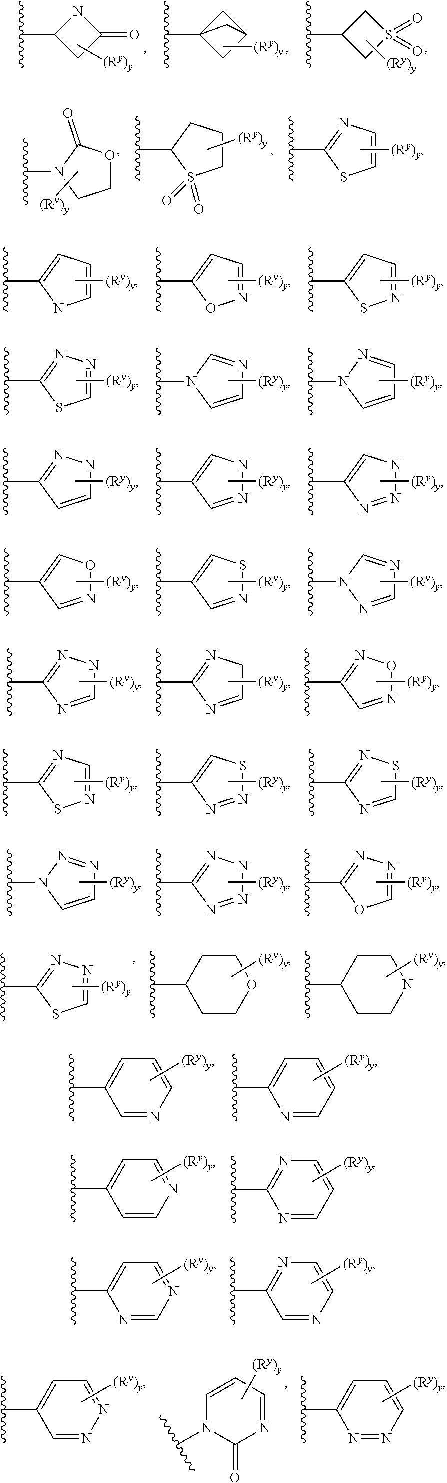

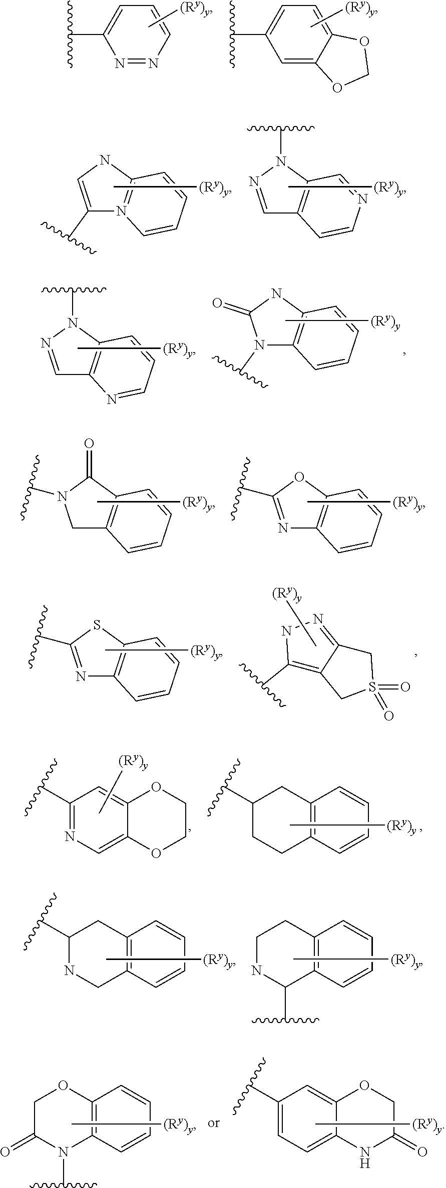

In some embodiment, Ring Y is

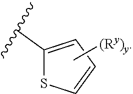

In some embodiment, Ring Y is as depicted in the compounds of Table 1A, below.

In some embodiments, Ring Y and its R^{y} substituents are

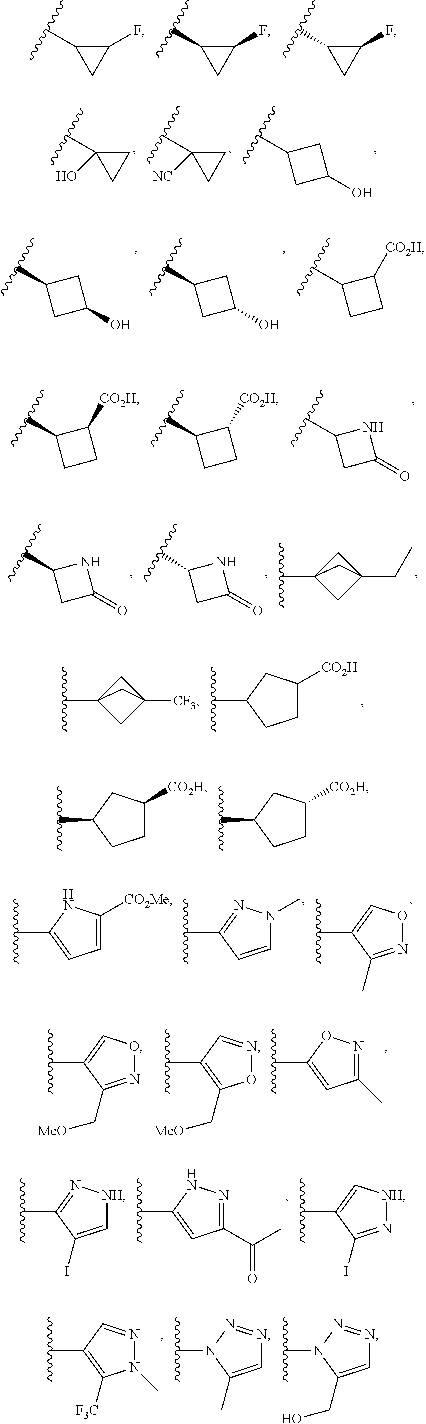

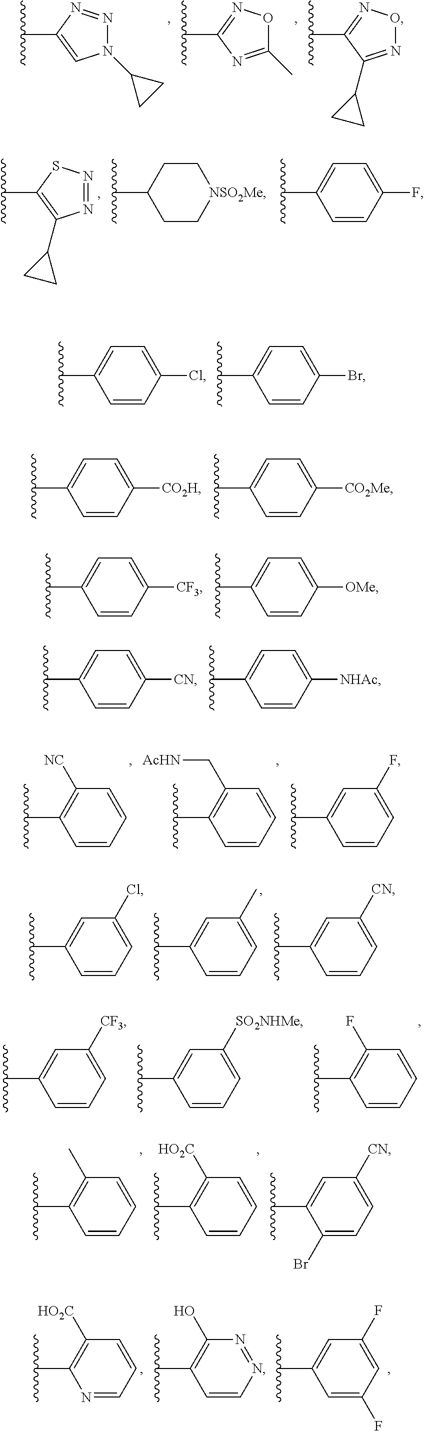

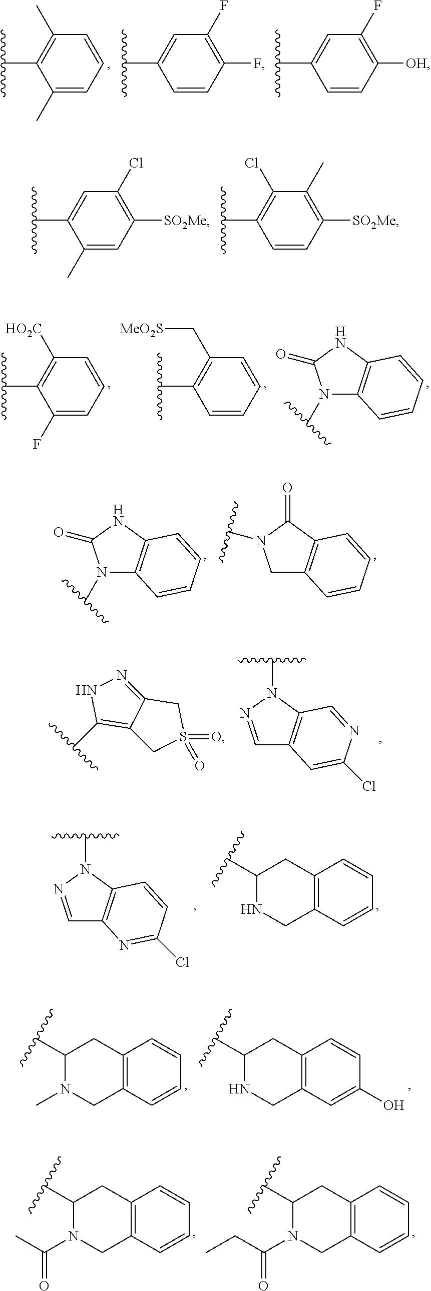

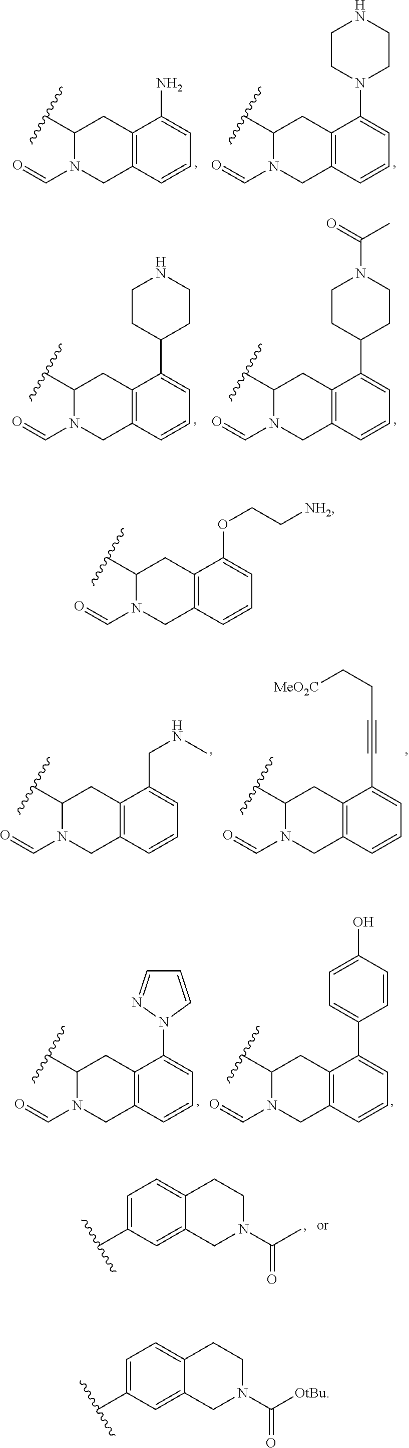

In some embodiments, Ring Y and its R^{y} substituents are

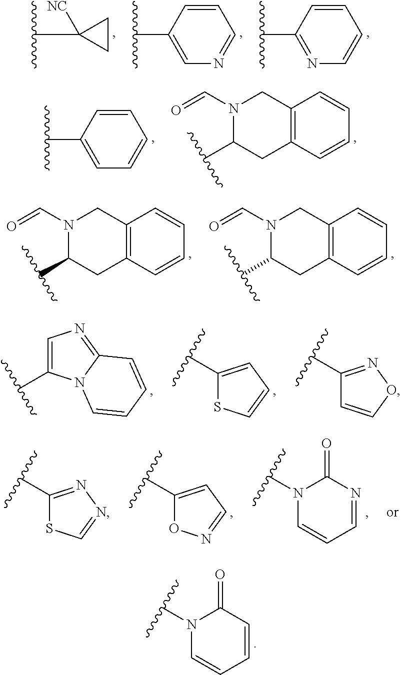

In some embodiments, Ring Y is

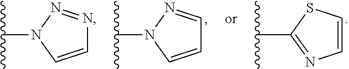

In some embodiments, Ring Y and its R^{y} substituents are

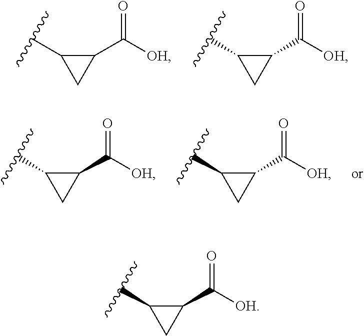 [SMILES: *C1CC1C(=O)O ; *[C@H]1C[C@H]1C(=O)O ; *[C@H]1C[C@@H]1C(=O)O ; *[C@@H]1C[C@H]1C(=O)O ; *[C@@H]1C[C@@H]1C(=O)O]

As described above and defined herein, R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, R^{x} and R^{y} are independently selected from R^{A}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

As described above and defined herein, each R^{w} is independently selected from hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, each R^{w} is independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, each R^{w} is independently selected from R^{A}, R^{B}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, one or more of R^{w}, R^{x}, and R^{y} is hydrogen. In some embodiments, one or more of R^{w}, R^{x}, and R^{y} is R^{A}. In some embodiments, one or more of R^{w}, R^{x}, and R^{y} is halogen. In some embodiments, one or more of R^{w}, R^{x}, and R^{y} is —CN. In some embodiments, one or more of R^{w}, R^{x}, and R^{y} is —NO₂. In some embodiments, one or more of R^{w}, R^{x}, and R^{y} is —OR. In some embodiments, one or more of R^{w}, R^{x}, and R^{y} is —SR. In some embodiments, one or more of R^{w}, R^{x}, and R^{y} is —NR₂. In some embodiments, one or more of R^{w}, R^{x}, and R^{y} is —SiR₃. In some embodiments, one or more of R^{w}, R^{x}, and R^{y} is —S(O)₂R. In some embodiments, one or more of R^{w}, R^{x}, and R^{y} is —S(O)₂NR₂. In some embodiments, one or more of R^{w}, R^{x}, and R^{y} is —S(O)(NR)R. In some embodiments, one or more of R^{w}, R^{x}, and R^{y} is —S(O)R. In some embodiments, one or more of R^{w}, R^{x}, and R^{y} is —C(O)R. In some embodiments, one or more of R^{w}, R^{x}, and R^{y} is —C(O)OR. In some embodiments, one or more of R^{w}, R^{x}, and R^{y} is —C(O)NR₂. In some embodiments, one or more of R^{w}, R^{x}, and R^{y} is —C(O)NROR. In some embodiments, one or more of R^{w}, R^{x}, and R^{y} is —OC(O)R. In some embodiments, one or more of R^{w}, R^{x}, and R^{y} is —OC(O)NR₂. In some embodiments, one or more of R^{w}, R^{x}, and R^{y} is —OP(O)R₂. In some embodiments, one or more of R^{w}, R^{x}, and R^{y} is —OP(O)(OR)₂. In some embodiments, one or more of R^{w}, R^{x}, and R^{y} is —NRC(O)OR. In some embodiments, one or more of R^{w}, R^{x}, and R^{y} is —NRC(O)R. In some embodiments, one or more of R^{w}, R^{x}, and R^{y} is —NRC(O)N(R)₂. In some embodiments, one or more of R^{w}, R^{x}, and R^{y} is —NRS(O)₂R.

In some embodiments, R^{w}—C(O)OR. In some embodiments, R^{w} is —C(O)NR₂. In some embodiments, R^{w} is an optionally substituted phenyl. In some embodiments, R^{w} is an optionally substituted 5-6 membered heteroaryl ring having 1-4 heteroatoms independently selected from nitrogen, oxygen, and sulfur.

In some embodiments, R^{w} is R^{B}. In some embodiments, R^{w} is R^{A}.

In some embodiments, R^{w} is fluoro, chloro, or bromo.

In some embodiments, R^{w} is —C(O)NHR. In some embodiments, R^{w} is —C(O)NHR, wherein R of R^{w} is optionally substituted C₁₋₆ aliphatic. In some embodiments, R^{w} is —C(O)NHR, wherein R of R^{w} is C₁₋₆ aliphatic, optionally substituted with —CN.

In some embodiments, R^{w} is optionally substituted C₁₋₆ aliphatic. In some embodiments, R^{w} is C₁₋₆ aliphatic, optionally substituted with —C(O)N(R^{◯})₂. In some embodiments, R^{w} is C₁₋₆ aliphatic, optionally substituted with —NR^{◯}(O)N(R^{◯})₂. In some embodiments, R^{w} is

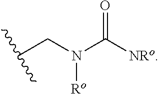

In some such embodiments, R^{◯} is hydrogen or C₁₋₆ aliphatic. In some embodiments, R^{w} is C₁₋₆ aliphatic optionally substituted with —OR^{◯}, wherein R^{◯} is hydrogen or C₁₋₆ aliphatic. In some embodiments, R^{w} is C₁₋₆ aliphatic optionally substituted with halogen (e.g., fluoro). In some embodiments, R^{w} is —CH₂F, —CHF₂, or —CF₃.

In some embodiments, R^{w} is C₁₋₆ aliphatic optionally substituted with halogen (e.g., fluoro). In some embodiments, R^{w} is

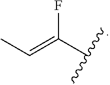 [SMILES: */C(F)=C/C]

In some embodiments, R^{w} is C₁₋₆ aliphatic optionally substituted with halogen (e.g., fluoro) or —NR^{◯}₂. In some embodiments, R^{w} is C₁₋₆ aliphatic optionally substituted with halogen (e.g., fluoro) and —NR^{◯}₂. In some embodiments, R^{w} is C₁₋₆ aliphatic optionally substituted with halogen (e.g., fluoro) or —NR^{◯}₂, wherein each R^{◯} is independently hydrogen or C₁₋₆ aliphatic. In some embodiments, R^{w} is C₁₋₆ aliphatic optionally substituted with halogen (e.g., fluoro) and —NR^{◯}₂, wherein each R^{◯} is independently hydrogen or C₁₋₆ aliphatic. In some embodiments, R^{w} is

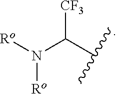

In some embodiments, R^{w} is an optionally substituted phenyl.

In some embodiments, R^{w} is an optionally substituted 3-7 membered saturated or partially unsaturated carbocyclic. In some embodiments, R^{w} is an optionally substituted 3-7 membered saturated or partially unsaturated carbocyclic. In some embodiments, R^{w} is an optionally substituted cyclopropyl. In some embodiments, R^{w} is an optionally substituted cyclobutyl. In some embodiments, R^{w} is an optionally substituted cyclopentyl. In some embodiments, R^{w} is an optionally substituted cyclohexyl. In some embodiments, R^{w} is an optionally substituted cyclopropenyl. In some embodiments, R^{w} is an optionally substituted cyclobutenyl. In some embodiments, R^{w} is an optionally substituted cyclopentenyl. In some embodiments, R^{w} is an optionally substituted cyclohexenyl.

In some embodiments, R^{w} is an optionally substituted 3-7 membered saturated or partially unsaturated heterocyclic ring having 1-2 heteroatoms independently selected from nitrogen, oxygen, and sulfur. In some embodiments, R^{w} is an optionally substituted 5-6 membered saturated or partially unsaturated heterocyclic ring having 1-2 heteroatoms independently selected from nitrogen, oxygen, and sulfur.

In some embodiments, R^{w} is an optionally substituted 4 membered saturated or partially unsaturated heterocyclic ring having 1-2 heteroatoms independently selected from nitrogen, oxygen, and sulfur. In some embodiments, R^{w} is an optionally substituted azetidinyl, oxetanyl, or thietanyl.

In some embodiments, R^{w} is an optionally substituted 5 membered saturated or partially unsaturated heterocyclic ring having 1-2 heteroatoms independently selected from nitrogen, oxygen, and sulfur. In some embodiments, R^{w} is an optionally substituted pyrrolidinyl, pyrrolinyl, pyrazolidinyl, pyrazolinyl, imidazolidinyl, or imidazolinyl. In some embodiments, R^{w} is an optionally substituted dihydropyridinyl, pyrrolidinyl, dihydrofuranyl, tetrahydrofuranyl, dihydrothiophenyl, or tetrahydrothiophenyl.

In some embodiments, R^{w} is an optionally substituted 6 membered saturated or partially unsaturated heterocyclic ring having 1-2 heteroatoms independently selected from nitrogen, oxygen, and sulfur. In some embodiments, R^{w} is an optionally substituted piperidinyl, piperazinyl, tetrahydropyranyl, 2H-pyranyl, 4H-pyranyl, 1,4-dioxanyl, 1,4-dioxinyl, thianyl, 2H-thiopyranyl, 4H-thiopyranyl, 1,3-dithanyl, 1,4-dithanyl, morpholinyl, or thiomorpholinyl. In some embodiments, R^{w} is an optionally substituted dihydropyridinyl, tetrahydropyridinyl, dihydropyranyl, tetrahydropyranyl, dihydrothiopyranyl, or tetrahydrothiopyranyl.

In some embodiments, R^{w} is an optionally substituted

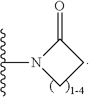

In some embodiments, R^{w} is an optionally substituted

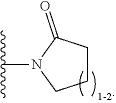

In some embodiments, R^{w} is an optionally substituted

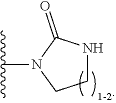

In some embodiments, R^{w} is

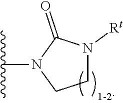

In some embodiments, R^{w} is optionally substituted

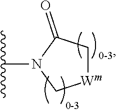

where W^{m} is O, S, C(O), or NR^{◯}, wherein R^{◯} is as described above and defined herein. In some embodiments, R^{w} is

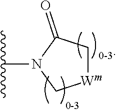

In some embodiments, R^{w} is optionally substituted

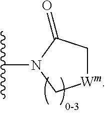

In some embodiments, R^{w} is

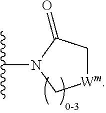

In some embodiments, R^{w} is

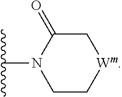

In some embodiments, R^{w} is

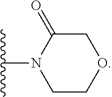 [SMILES: *N1CCOCC1=O]

In some embodiments, W^{m} is O. In some embodiments, W^{m} is S. In some embodiments, Wm is C(O). In some embodiments, Wm is NR^{◯}. In some embodiments, W^{m} is NR^{◯}, wherein R^{◯} is hydrogen or C₁₋₆ aliphatic.

In some embodiments, R^{w} is an optionally substituted 5-6 membered heteroaryl having 1-4 heteroatoms independently selected from nitrogen, oxygen, and sulfur. In some embodiments R^{w} is an optionally substituted 5 membered heteroaryl having 1-4 heteroatoms independently selected from nitrogen, oxygen, and sulfur. In some embodiments, R^{w} is an optionally substituted pyrazolyl, imidazolyl, triazolyl, or tetrazolyl. In some embodiments, R^{w} is an optionally substituted imidazolyl, optionally substituted with —C(O)N(R^{◯})₂. In some embodiments, R^{w} is an optionally substituted furanyl, thiophenyl, oxazolyl, isoxazolyl, isothiazolyl, thiazolyl, oxadiazolyl, or thiadiazolyl. In some embodiments, R^{w} is furanyl, optionally substituted with —C(O)N(R^{◯})₂.

In some embodiments R^{w} is an optionally substituted 6 membered heteroaryl having 1-4 heteroatoms independently selected from nitrogen, oxygen, and sulfur. In some embodiments R^{w} is an optionally substituted 6 membered heteroaryl having 1-4 nitrogen heteroatoms. In some embodiments, R^{w} is optionally substituted pyridinyl, pyrimidinyl, pyridazinyl, or triazinyl. In some embodiments, R^{w} is an optionally substituted pyridinonyl, pyrazinonyl, or pyrimidinoyl.

In some embodiments, R^{w} is —NHR, wherein R is an optionally substituted 5-6 membered heteroaryl having 1-4 heteroatoms independently selected from nitrogen, oxygen, and sulfur. In some embodiments, R^{w} is —NHR, wherein R is an optionally substituted 6 membered heteroaryl having 1-4 heteroatoms independently selected from nitrogen, oxygen, and sulfur.

In some embodiments, R^{w} is —S(O)₂NH₂. In some embodiments, R^{w} is —S(O)₂NHR, wherein R is an optionally substituted C₁₋₆ aliphatic. In embodiments, R^{w} is —S(O)₂NHR, wherein R is an optionally substituted phenyl, 4-7 membered saturated or partially unsaturated carbocyclic or heterocyclic ring having 1-2 heteroatoms independently selected from nitrogen, oxygen, and sulfur, or a 5-6 membered heteroaryl ring having 1-4 heteroatoms independently selected from nitrogen, oxygen, and sulfur.

In embodiments, R^{w} is —S(O)₂(NH)R. In some embodiments, R^{w} is —S(O)₂(NH)H. In some embodiments, R^{w} is —S(O)₂(NH)R, wherein R is an optionally substituted C₁₋₆ aliphatic. In some embodiments, R^{w} is —S(O)₂(NH)R, wherein R is an optionally substituted phenyl, 4-7 membered saturated or partially unsaturated carbocyclic or heterocyclic ring having 1-2 heteroatoms independently selected from nitrogen, oxygen, and sulfur, or a 5-6 membered heteroaryl ring having 1-4 heteroatoms independently selected from nitrogen, oxygen, and sulfur.

In some embodiments, R^{w} is —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, or —NRS(O)₂R. In some embodiments, R^{w} is —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R. In some embodiments, R^{w} is —S(O)R, —S(O)₂R, —S(O)(NR)R, —S(O)₂NR₂, or —NRS(O)₂R. In some embodiments, R^{w} is —C(O)R, —C(O)OR, —C(O)NR₂, —C(O)NROR, —OC(O)R, —OC(O)NR₂, —NRC(O)OR, —NRC(O)R, or —NRC(O)N(R)₂. In some embodiments, R^{w} is —S(O)R, —S(O)₂R, —S(O)(NR)R, —S(O)₂NR₂, —C(O)R, —C(O)OR, —C(O)NR₂, or —C(O)NROR. In some embodiments, R^{w} is —C(O)R, —C(O)OR, or —C(O)NR₂.

In some embodiments, R^{w} is —C(O)H. In some embodiments, R^{w} is —C(O)R, wherein R is of R^{w} is optionally substituted C₁₋₆ aliphatic. In some embodiments, R^{w} is —C(O)R, wherein R is of R^{w} is optionally substituted phenyl. In some embodiments, R^{w} is —C(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)R, wherein R is of R^{w} is an optionally substituted 5-6 membered heteroaryl ring having 1-4 heteroatoms independently selected from nitrogen, oxygen, and sulfur.

In some embodiments, R^{w} is —C(O)R, wherein R is of R^{w} is optionally substituted C₁₋₆ aliphatic. In some embodiments, R^{w} is —C(O)R, wherein R is of R^{w} is optionally substituted phenyl. In some embodiments, R^{w} is —C(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)R, wherein R is of R^{w} is an optionally substituted 5-6 membered heteroaryl ring having 1-4 heteroatoms independently selected from nitrogen, oxygen, and sulfur.

In some embodiments, R^{w} is —C(O)OH. In some embodiments, R^{w} is —C(O)OR, wherein R is of R^{w} is optionally substituted C₁₋₆ aliphatic. In some embodiments, R^{w} is —C(O)OR, wherein R is of R^{w} is optionally substituted phenyl. In some embodiments, R^{w} is —C(O)OR, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)OR, wherein R is of R^{w} is an optionally substituted 5-6 membered heteroaryl ring having 1-4 heteroatoms independently selected from nitrogen, oxygen, and sulfur.

In some embodiments, R^{w} is —C(O)NH₂. In some embodiments, R^{w} is —C(O)NR₂, wherein each R is of R^{w} is independently hydrogen or an optionally substituted C₁₋₆ aliphatic. In some embodiments, R^{w} is —C(O)NR₂, wherein each R is of R^{w} is independently hydrogen or an optionally substituted phenyl. In some embodiments, R^{w} is —C(O)NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —C(O)NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —C(O)NHR, wherein R is an optionally substituted C₁₋₆ aliphatic. In some embodiments, R^{w} is —C(O)NHR, wherein R is C₁₋₆ aliphatic. In some embodiments, R^{w} is —C(O)NHR, wherein R is methyl, ethyl, or cyclopropyl. In some embodiments, R^{w} is —C(O)NR₂, wherein the two R groups of R^{w} are taken together with their intervening atoms to form a 3-7 membered saturated or partially unsaturated monocyclic ring having 0-3 heteroatoms, in addition to the atom or adjacent atoms to which they are attached, independently selected form nitrogen, oxygen, and sulfur. In some embodiments, R^{w} is —C(O)NR₂, wherein the two R groups of R^{w} are taken together with their intervening atoms to form a 3-7 membered saturated or partially unsaturated monocyclic ring having 0 heteroatoms, in addition to the atom or adjacent atoms to which they are attached. In some embodiments, R^{w} is —C(O)NR₂, wherein the two R groups of R^{w} are taken together with their intervening atoms to form an aziridinyl, azetidinyl, diazetidinyl, pyrrolidinyl, or piperidinyl.

In some embodiments, R^{w} is —C(O)NROR, wherein each R is of R^{w} is independently hydrogen or an optionally substituted C₁₋₆ aliphatic. In some embodiments, R^{w} is —C(O)NROR, wherein each R is of R^{w} is independently hydrogen or an optionally substituted phenyl. In some embodiments, R^{w} is —C(O)NROR, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —C(O)NROR, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —OC(O)H. In some embodiments, R^{w} is —OC(O)R, wherein R is of R^{w} is optionally substituted C₁₋₆ aliphatic. In some embodiments, R^{w} is —OC(O)R, wherein R is of R^{w} is optionally substituted phenyl. In some embodiments, R^{w} is —OC(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —OC(O)R, wherein R is of R^{w} is an optionally substituted 5-6 membered heteroaryl ring having 1-4 heteroatoms independently selected from nitrogen, oxygen, and sulfur.

In some embodiments, R^{w} is —OC(O)NR₂, wherein each R is of R^{w} is independently hydrogen or an optionally substituted C₁₋₆ aliphatic. In some embodiments, R^{w} is —OC(O)NR₂, wherein each R is of R^{w} is independently hydrogen or an optionally substituted phenyl. In some embodiments, R^{w} is —OC(O)NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —OC(O)NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRC(O)OR, wherein each R is of R^{w} is independently hydrogen or an optionally substituted C₁₋₆ aliphatic. In some embodiments, R^{w} is —NRC(O)OR, wherein each R is of R^{w} is independently hydrogen or an optionally substituted phenyl. In some embodiments, R^{w} is —NRC(O)OR, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)OR, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRC(O)R, wherein each R is of R^{w} is independently hydrogen or an optionally substituted C₁₋₆ aliphatic. In some embodiments, R^{w} is —NRC(O)R, wherein each R is of R^{w} is independently hydrogen or an optionally substituted phenyl. In some embodiments, R^{w} is —NRC(O)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRC(O)N(R)₂, wherein each R is of R^{w} is independently hydrogen or an optionally substituted C₁₋₆ aliphatic. In some embodiments, R^{w} is —NRC(O)N(R)₂, wherein each R is of R^{w} is independently hydrogen or an optionally substituted phenyl. In some embodiments, R^{w} is —NRC(O)N(R)₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)N(R)₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRS(O)₂R, wherein each R is of R^{w} is independently hydrogen or an optionally substituted C₁₋₆ aliphatic. In some embodiments, R^{w} is —NRS(O)₂R, wherein each R is of R^{w} is independently hydrogen or an optionally substituted phenyl. In some embodiments, R^{w} is —NRS(O)₂R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRS(O)₂R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —S(O)H. In some embodiments, R^{w} is —S(O)R, wherein R is of R^{w} is optionally substituted C₁₋₆ aliphatic. In some embodiments, R^{w} is —S(O)R, wherein R is of R^{w} is optionally substituted phenyl. In some embodiments, R^{w} is —S(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —S(O)R, wherein R is of R^{w} is an optionally substituted 5-6 membered heteroaryl ring having 1-4 heteroatoms independently selected from nitrogen, oxygen, and sulfur.

In some embodiments, R^{w} is —S(O)₂H. In some embodiments, R^{w} is —S(O)₂R, wherein R is of R^{w} is optionally substituted C₁₋₆ aliphatic. In some embodiments, R^{w} is —S(O)₂R, wherein R is of R^{w} is optionally substituted phenyl. In some embodiments, R^{w} is —S(O)₂R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —S(O)₂R, wherein R is of R^{w} is an optionally substituted 5-6 membered heteroaryl ring having 1-4 heteroatoms independently selected from nitrogen, oxygen, and sulfur.

In some embodiments, R^{w} is —S(O)(NR)R, wherein each R is of R^{w} is independently hydrogen or optionally substituted C₁₋₆ aliphatic. In some embodiments, R^{w} is —S(O)(NR)R, wherein each R is of R^{w} is independently hydrogen or optionally substituted phenyl. In some embodiments, R^{w} is —S(O)(NR)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —S(O)(NR)R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —S(O)₂NR₂, wherein each R is of R^{w} is independently hydrogen or an optionally substituted C₁₋₆ aliphatic. In some embodiments, R^{w} is —S(O)₂NR₂, wherein each R is of R^{w} is independently hydrogen or an optionally substituted phenyl. In some embodiments, R^{w} is —S(O)₂NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —S(O)₂NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is an optionally substituted 3-7 membered saturated or partially unsaturated carbocyclic or heterocyclic ring having 1-2 heteroatoms independently selected from nitrogen, oxygen, and sulfur. In some embodiments, R^{w} is an optionally substituted 3-7 membered saturated or partially unsaturated heterocyclic ring having 1-2 heteroatoms independently selected from nitrogen, oxygen, and sulfur, and is substituted with ═O. In some embodiments, R^{w} is an optionally substituted 5-6 membered monocyclic heteroaryl ring having 1-4 heteroatoms independently selected from nitrogen, oxygen, and sulfur. In some embodiments, R^{w} is an optionally substituted 5-6 membered monocyclic heteroaryl ring having 1-4 heteroatoms independently selected from nitrogen, oxygen, and sulfur, and is substituted with ═O. In some embodiments, R^{w} is an optionally substituted:

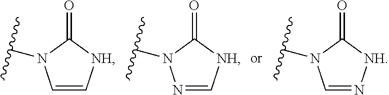

In some embodiments, R^{w} is:

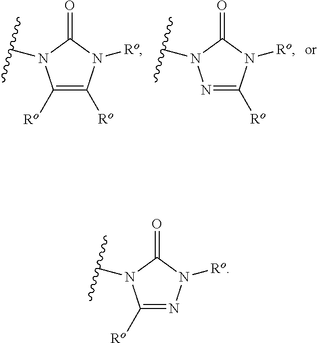

In some such embodiments, R^{◯} is hydrogen or C₁₋₆ aliphatic.

In some embodiments, R^{w} is:

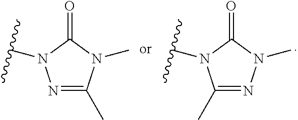

In some embodiments, R^{w} is an optionally substituted:

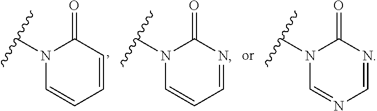

In some embodiments, R^{w} is:

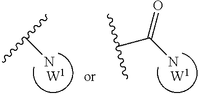

wherein Ring W¹ is an optionally substituted 3-7 membered saturated or partially unsaturated heterocyclic ring having 1 additional heteroatom selected from nitrogen, oxygen, and sulfur, or a 5-6 membered monocyclic heteroaryl ring having 1-3 additional heteroatoms independently selected from nitrogen, oxygen, and sulfur.

As defined above and described herein, Ring W¹ is an optionally substituted 3-7 membered saturated or partially unsaturated heterocyclic ring having 1 additional heteroatom selected from nitrogen, oxygen, and sulfur, or a 5-6 membered monocyclic heteroaryl ring having 1-3 additional heteroatoms independently selected from nitrogen, oxygen, and sulfur. In some embodiments, Ring W¹ is an optionally substituted 3-7 membered saturated or partially unsaturated heterocyclic ring having 1 additional heteroatom selected from nitrogen, oxygen, and sulfur. In some embodiments, Ring W¹ is an optionally substituted 5-6 membered saturated or partially unsaturated heterocyclic ring having 1 additional heteroatom selected from nitrogen, oxygen, and sulfur. In some embodiments, Ring W¹ is an optionally substituted 5-6 membered monocyclic heteroaryl ring having 1-3 additional heteroatoms independently selected from nitrogen, oxygen, and sulfur. In some embodiments, Ring W¹ is an optionally substituted 5 membered monocyclic heteroaryl ring having 1-3 additional heteroatoms independently selected from nitrogen, oxygen, and sulfur. In some embodiments, Ring W¹ is an optionally substituted 6 membered monocyclic heteroaryl ring having 1-3 additional heteroatoms independently selected from nitrogen, oxygen, and sulfur.

In some embodiments, R^{w} is:

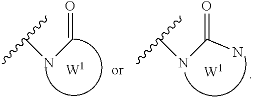

In some embodiments, R^{w} is:

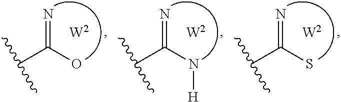

wherein Ring W² is an optionally substituted 3-7 membered partially unsaturated heterocyclic ring, or a 5-6 membered monocyclic heteroaryl ring having 1-2 additional heteroatoms independently selected from nitrogen, oxygen, and sulfur.

As defined above and described herein, Ring W² is an optionally substituted 3-7 membered partially unsaturated heterocyclic ring, or a 5-6 membered monocyclic heteroaryl ring having 1-2 additional heteroatoms independently selected from nitrogen, oxygen, and sulfur. In some embodiments, Ring W² is an optionally substituted 3-7 membered partially unsaturated heterocyclic ring. In some embodiments, Ring W² is an optionally substituted 5-6 membered partially unsaturated heterocyclic ring. In some embodiments, Ring W² is an optionally substituted 5-6 membered monocyclic heteroaryl ring having 1-2 additional heteroatoms independently selected from nitrogen, oxygen, and sulfur. In some embodiments, Ring W² is a 5 membered monocyclic heteroaryl ring having 1-2 additional heteroatoms independently selected from nitrogen, oxygen, and sulfur. In some embodiments, Ring W² is an optionally substituted oxazolyl, imidazolyl, thiazolyl, 1,3,4-thiadiazolyl, 2-imidazolinyl, 1,2,4-triazolyl, 1,2,4-oxadiazolyl, or 1,3,4-oxadiazolyl.

In some embodiments, R^{w} is an optionally substituted ring selected from:

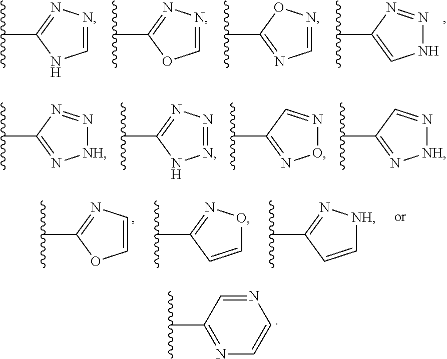

In some embodiments, R^{w} is

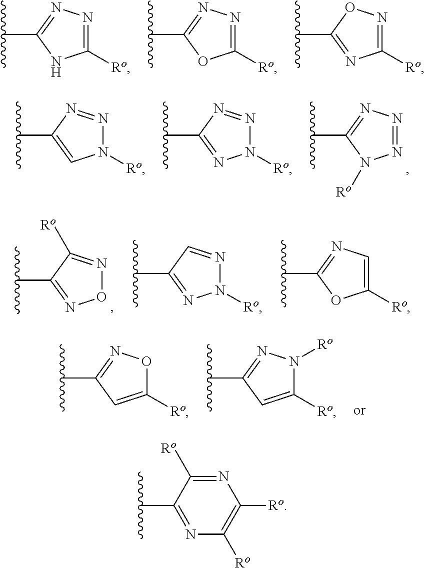

wherein each R^{◯} is a defined above and described herein (e.g., hydrogen or C₁₋₆ aliphatic).

In some embodiments, R^{w} is:

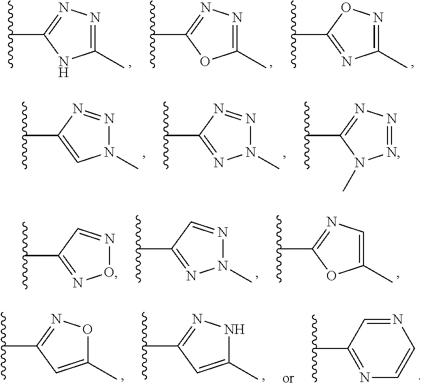

In some embodiments R^{w} is an optionally substituted 7-11 membered bicyclic heterocyclic ring having 1-4 heteroatoms independently selected from nitrogen, oxygen, and sulfur. In some embodiments R^{w} is an optionally substituted 9-10 membered bicyclic heterocyclic ring having 1-4 heteroatoms independently selected from nitrogen, oxygen, and sulfur. In some embodiments R^{w} is an optionally substituted benzo[d][1,3]dioxolyl. In some embodiments R^{w} is an optionally substituted an 11 membered bicyclic heterocyclic ring having 1-4 heteroatoms independently selected from nitrogen, oxygen, and sulfur.

In some embodiments R^{w} is an optionally substituted 8-11 membered bicyclic aryl or heteroaryl having 1-4 heteroatoms independently selected from nitrogen, oxygen and sulfur. In some embodiments, R^{w} is an optionally substituted naphthalenyl. In some embodiments, R^{w} is an optionally substituted dihydrobenzodioxepinyl. In some embodiments, R^{w} is an optionally substituted indenyl or dihydroindenyl.

In some embodiments, R^{w} is an optionally substituted 9-10 membered bicyclic heteroaryl having 1-4 heteroatoms independently selected from nitrogen, oxygen and sulfur.

In some embodiments R^{w} is an optionally substituted 9-membered bicyclic heteroaryl having 1-4 heteroatoms independently selected from nitrogen, oxygen and sulfur. In some embodiments R^{w} is an optionally substituted indolyl, benzofuranyl, benzothiophenyl, benzimidazolyl, benzoxazolyl, thienopyridinyl, pyrrolo[3,2-b]pyridinyl, pyrrolo[2,3,-b]pyridinyl, pyrazolyl[1,5-a]pyridinyl, or imidazo[1,2-a]pyridinyl, azaindazolyl (e.g., 4-, 5-, 6-, or 7-azaindazolyl), pyrrolol[2,3-c]pyridinyl or indolizinyl.

In some embodiments R^{w} is an optionally substituted 10-membered bicyclic heteroaryl having 1-4 heteroatoms independently selected from nitrogen, oxygen and sulfur. In some embodiments R^{w} is an optionally substituted quinolinyl, isoquinolinyl, quinolizinyl, quinoxalinyl, phthalazinyl, quinazolinyl, cinnolinyl, or 1,8-naphthyridinyl.

In some embodiments, R^{w} is fluoro, chloro, —CN, —OH, —OMe, —OCH₂CO₂Me, —CO₂H, —C(O)NH₂, —C(O)NHMe, —C(O)NHEt, —C(O)NHnPr, —C(O)NHCH₂CH₂OH, —C(O)NMe₂, —C(O)N(Me)Et, —C(O)N(Me)nPr, —CH₂NHMe,

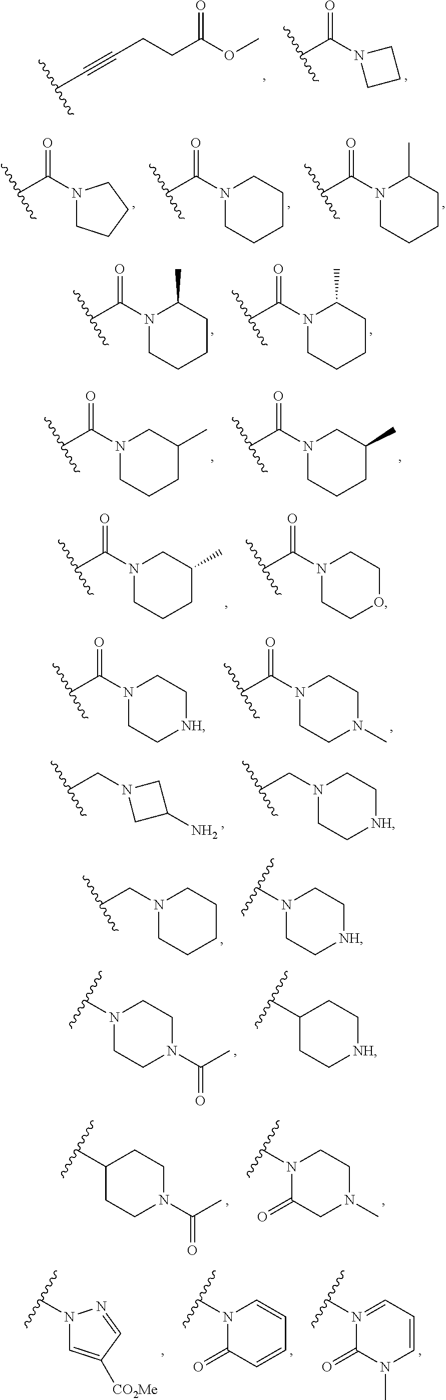

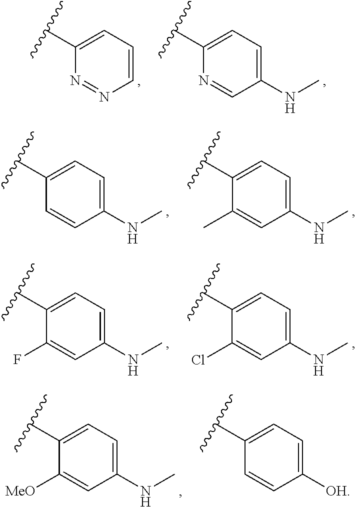

In some embodiments, R^{w} is fluoro, chloro, —CN, methyl, —CF₃, —CHF₂, —OH, —OMe, —OCH₂CO₂Me, —CO₂H, —C(O)NH₂, —C(O)NHMe, —C(O)NHEt, —C(O)NHnPr, —C(O)NHCH₂CH₂OH, —C(O)NMe₂, —C(O)N(Me)Et, —C(O)N(Me)nPr, —CH₂NHMe,

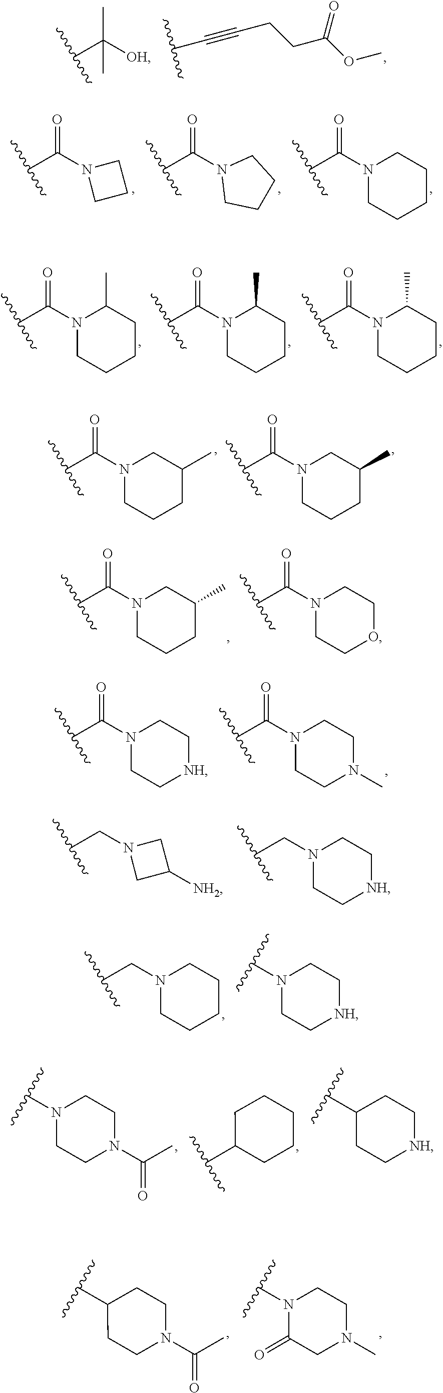

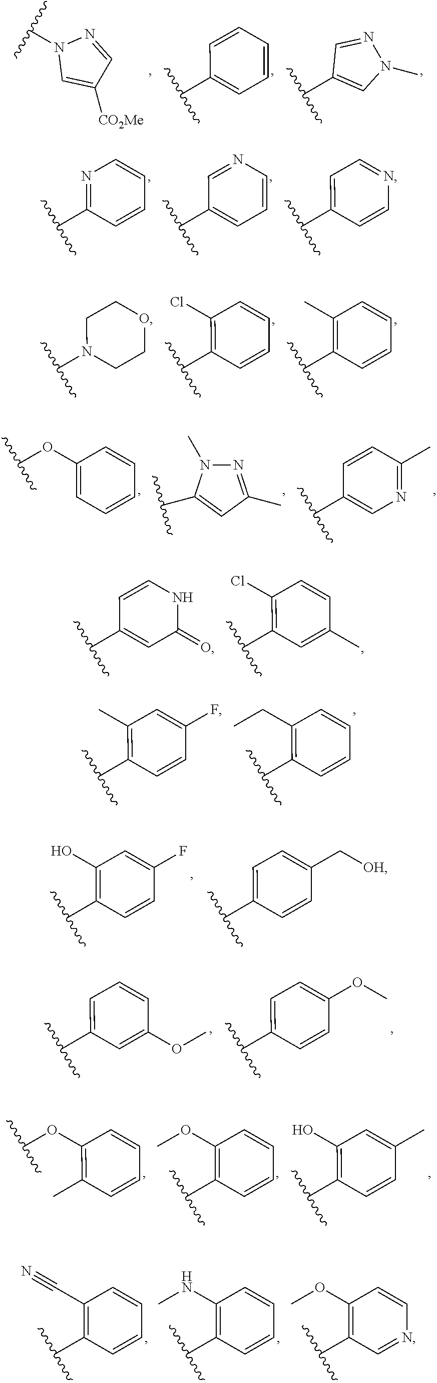

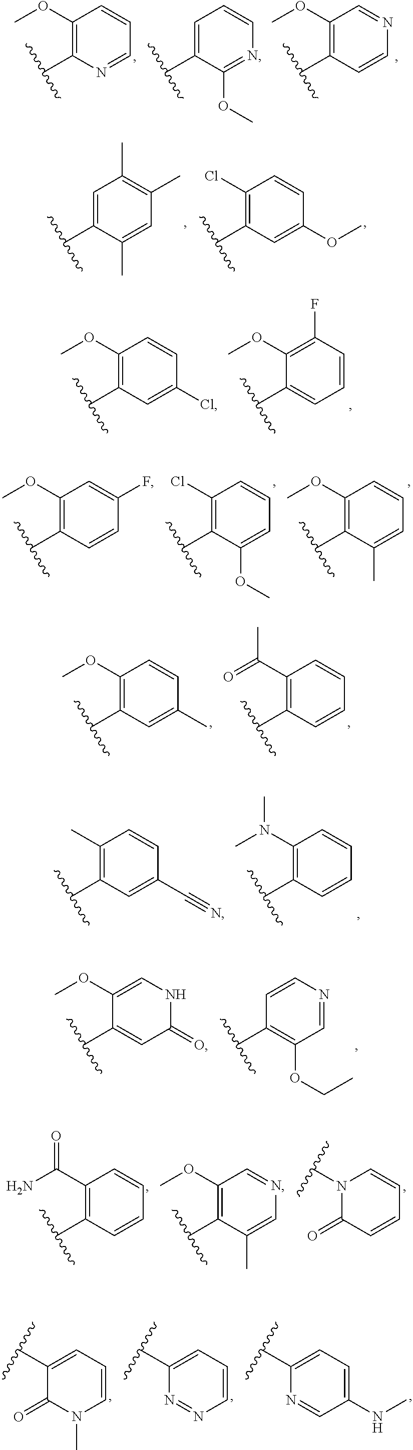

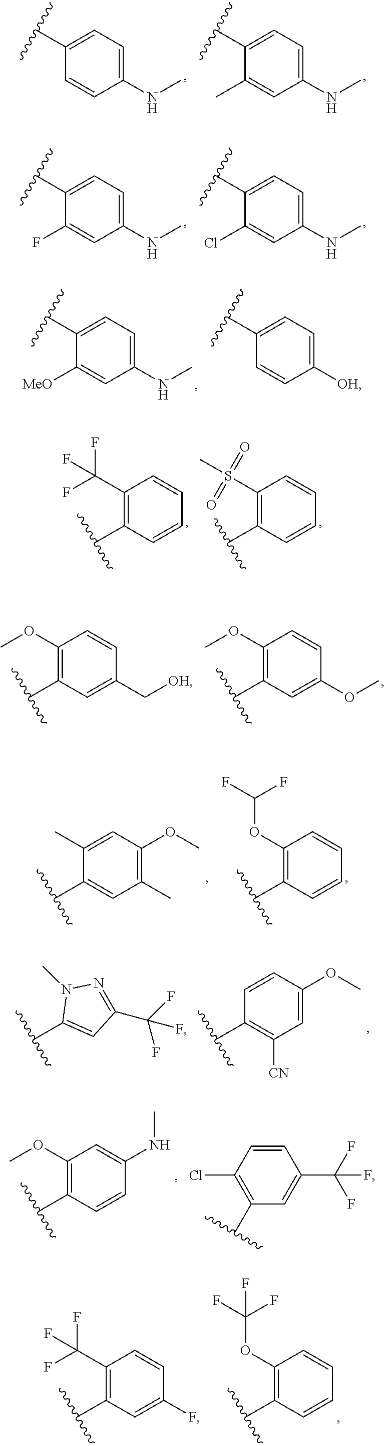

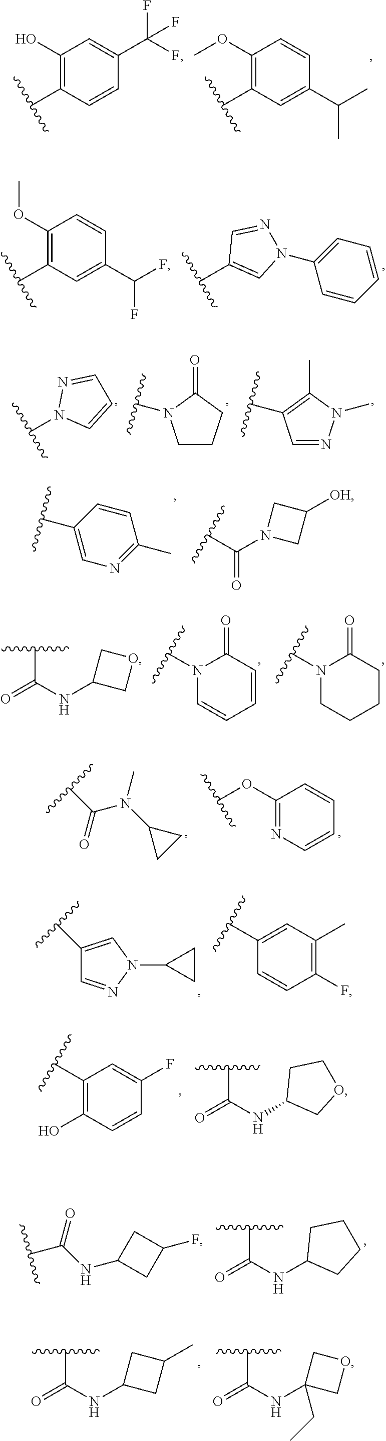

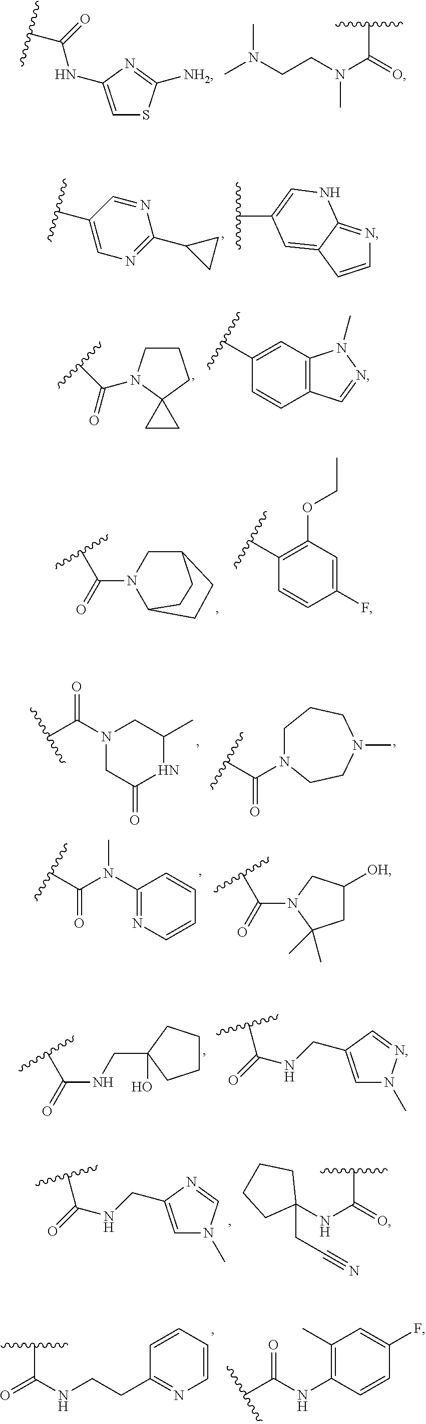

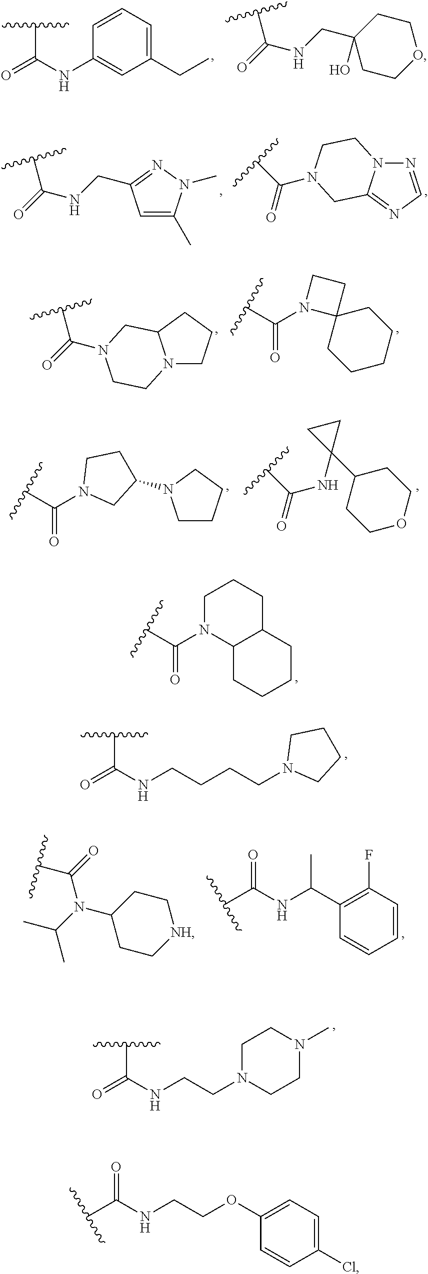

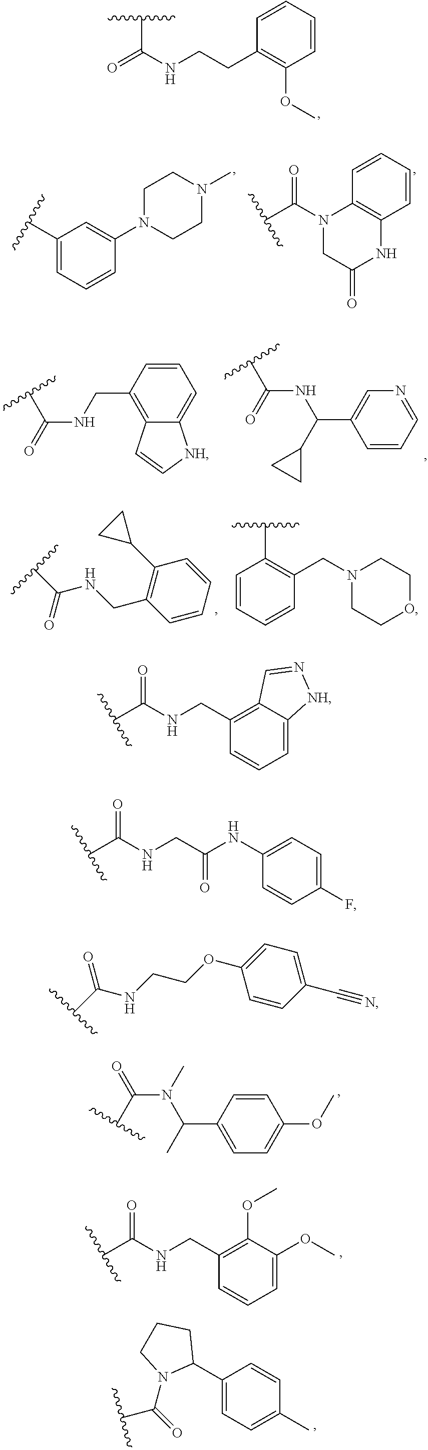

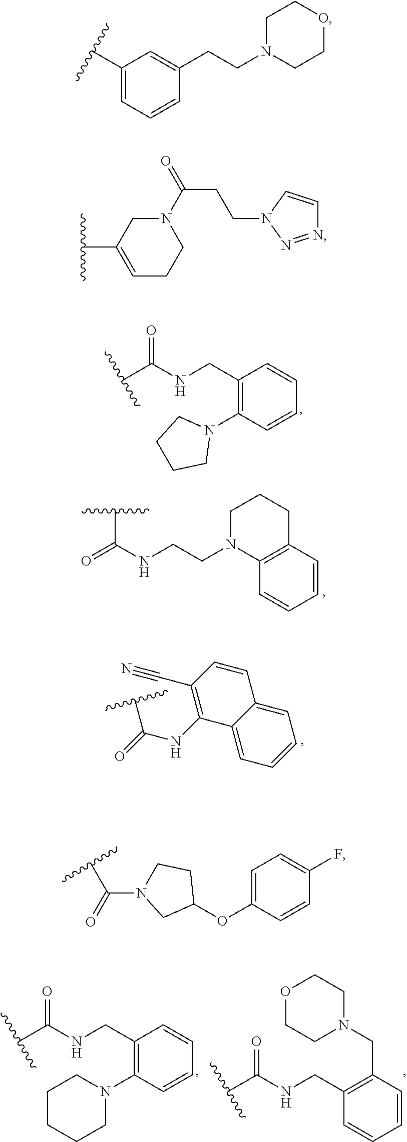

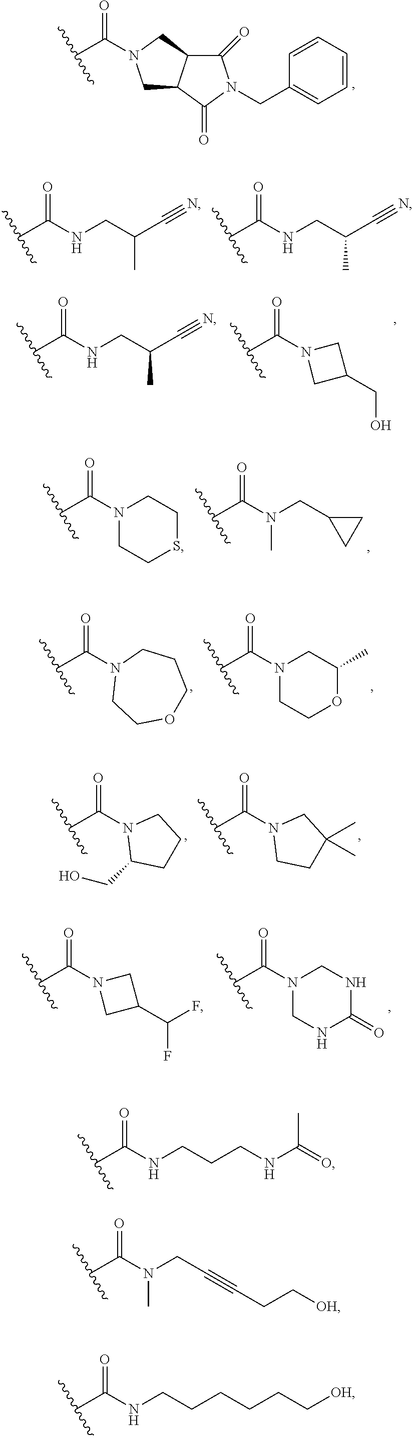

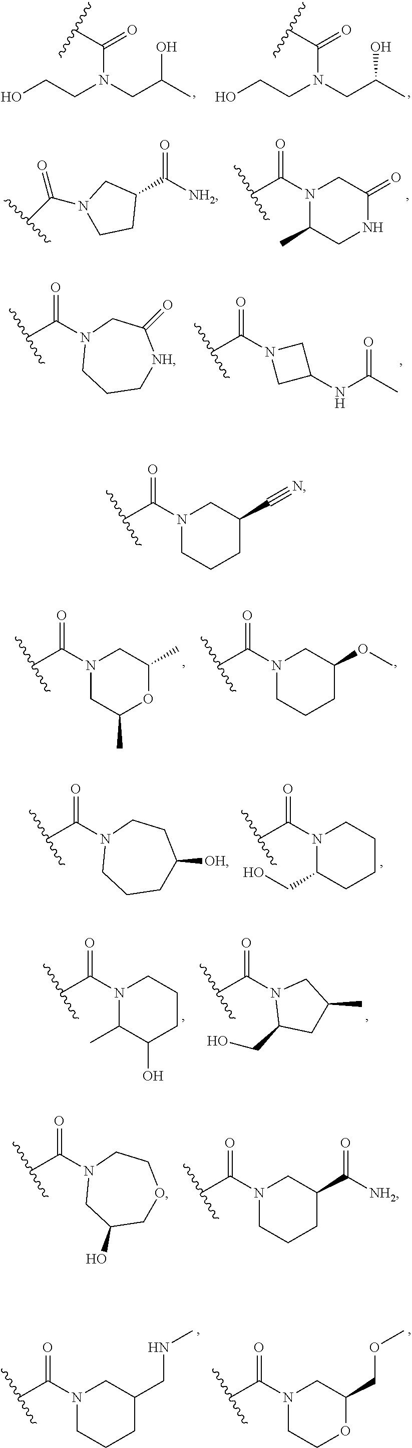

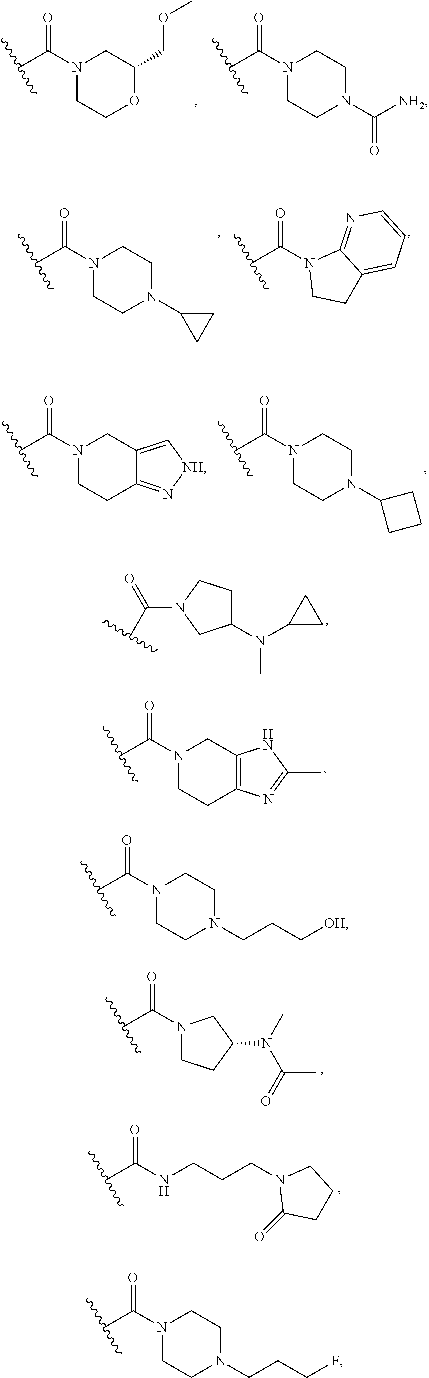

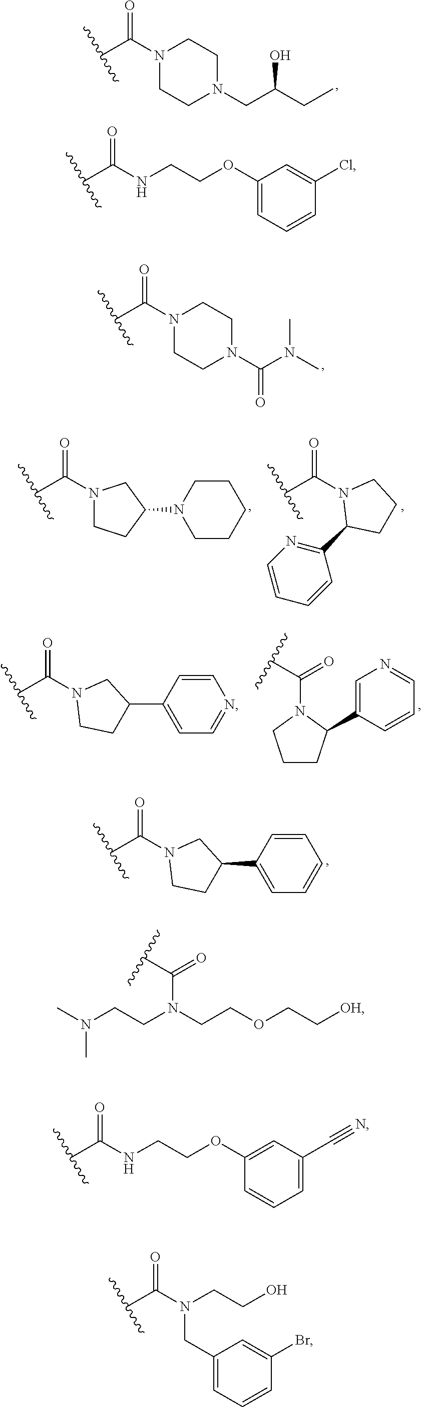

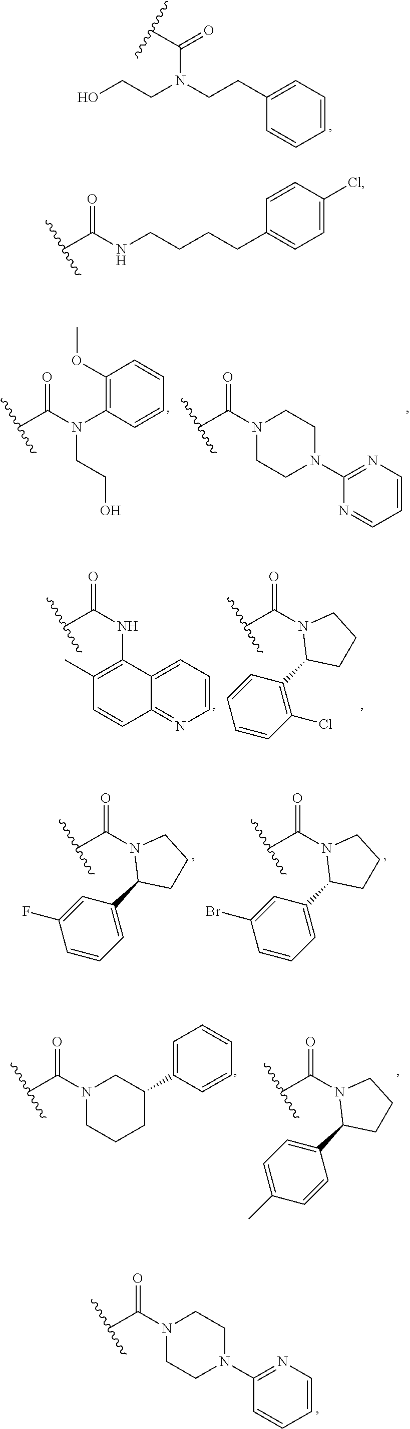

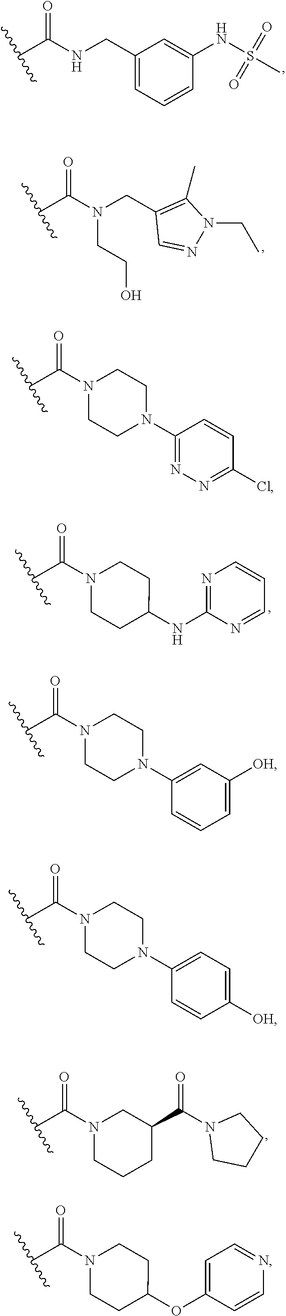

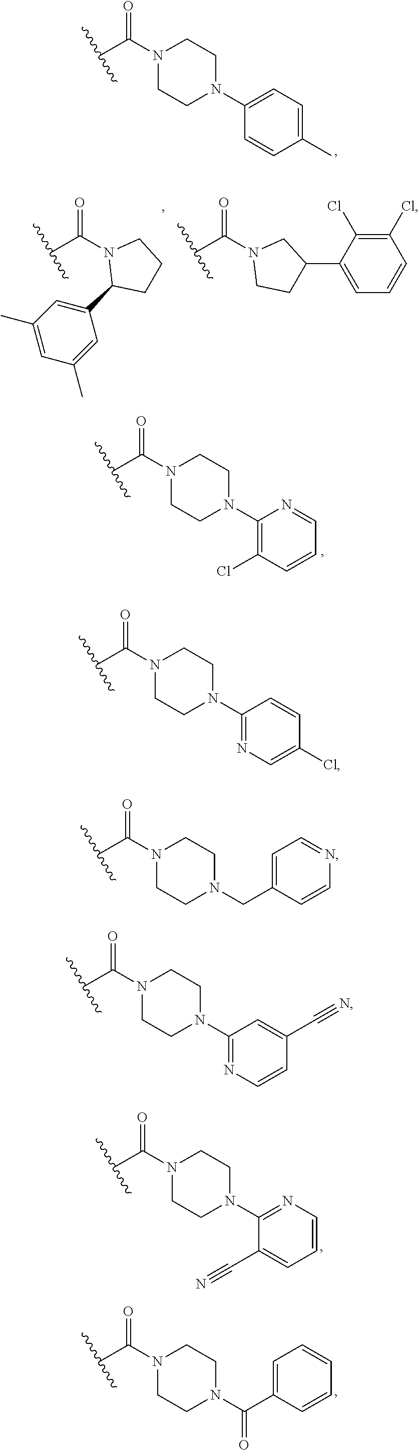

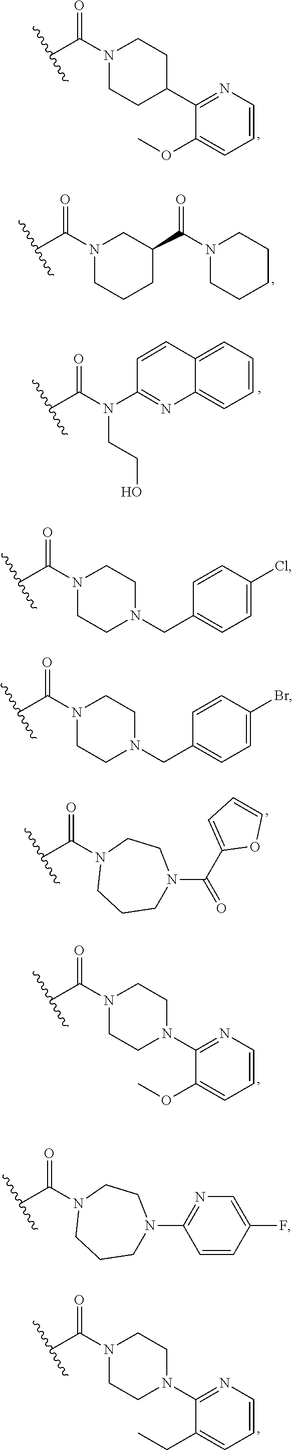

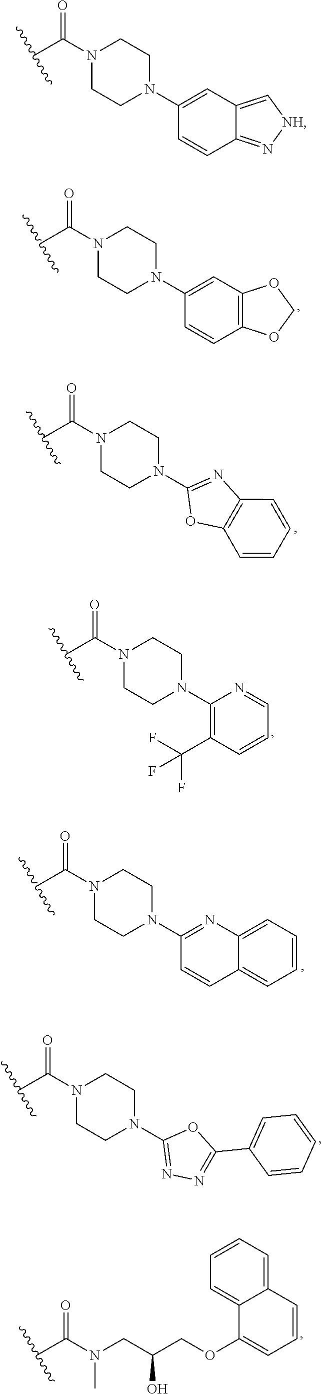

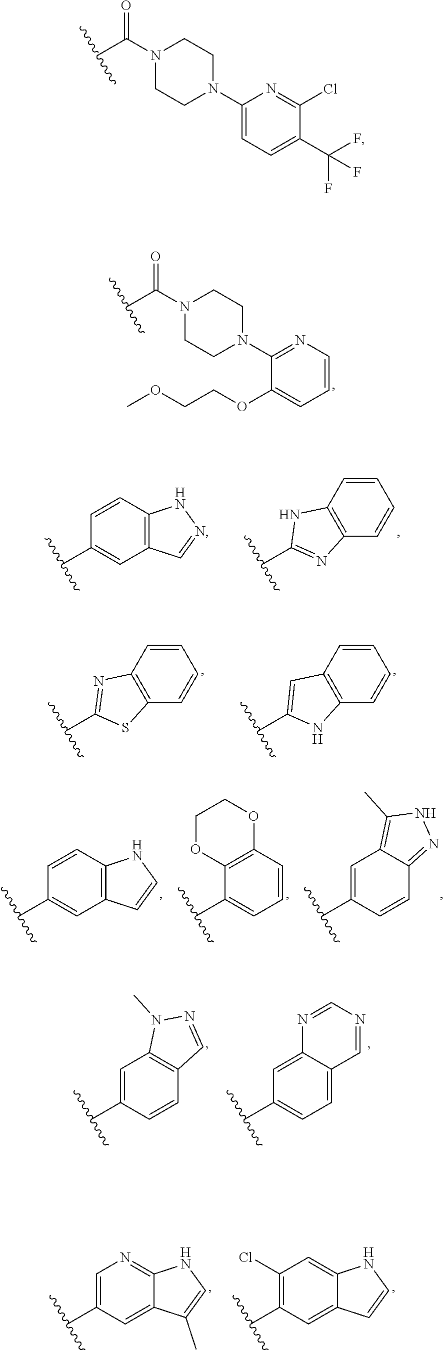

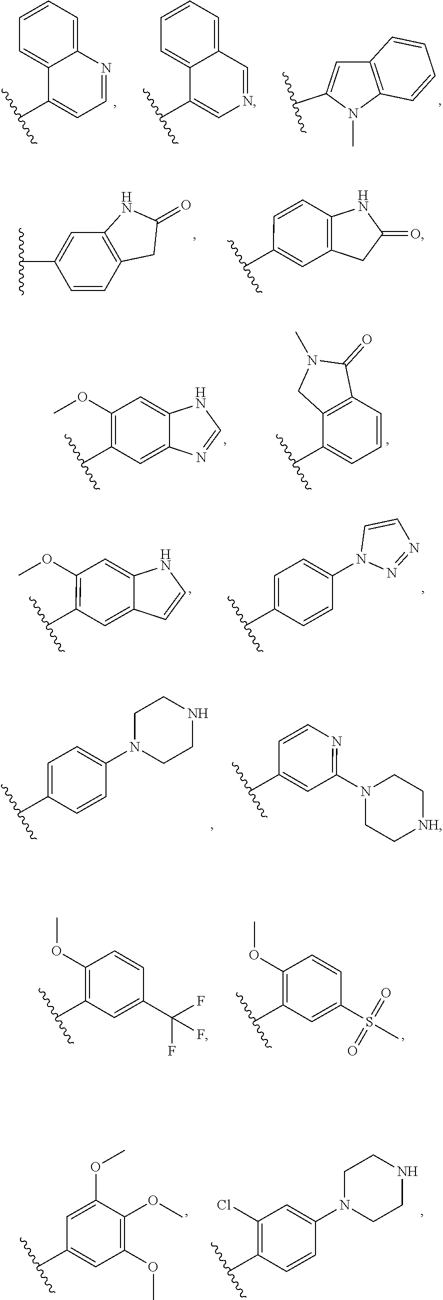

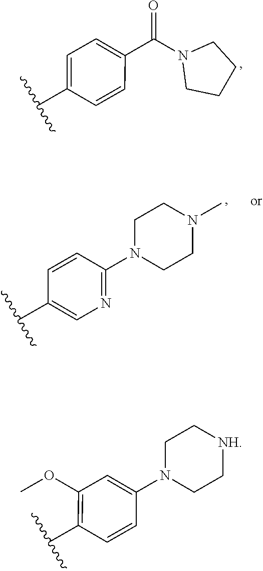

In some embodiments, R^{w} is

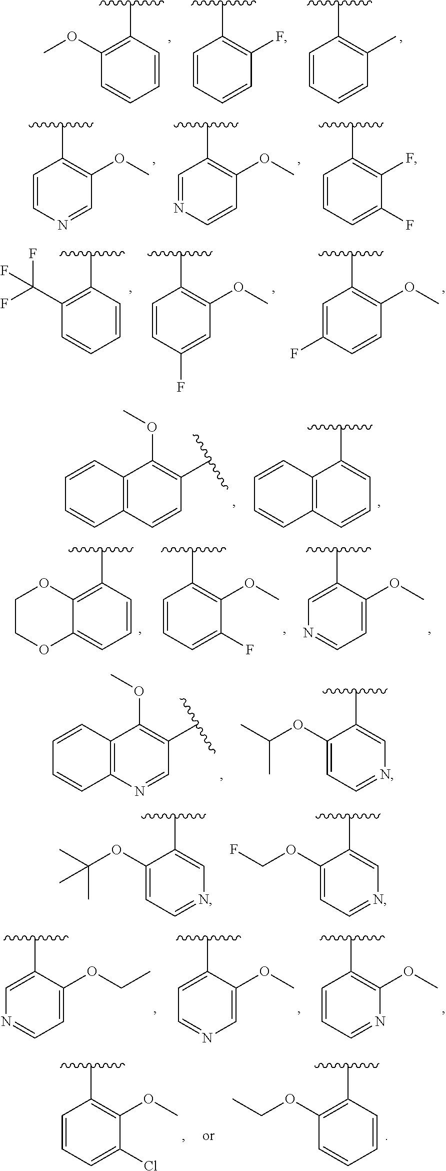

In some embodiments R^{w} is —S(O)₂NH₂, —S(O)₂N(CH₃)₂, —S(O)(NH)CH₃,

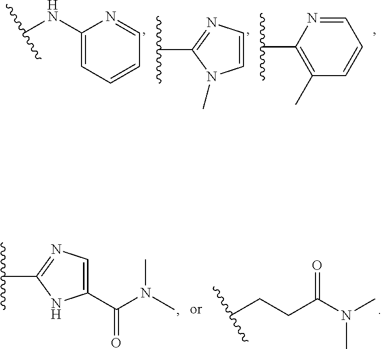

In some embodiments, R^{w} is

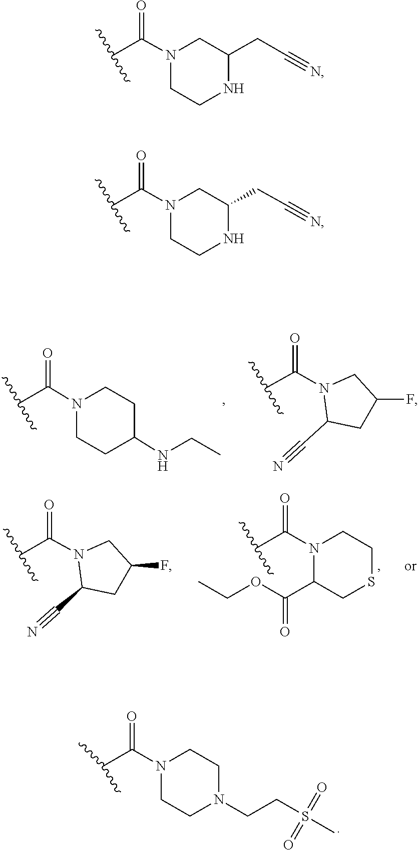

In some embodiments, R^{w} is

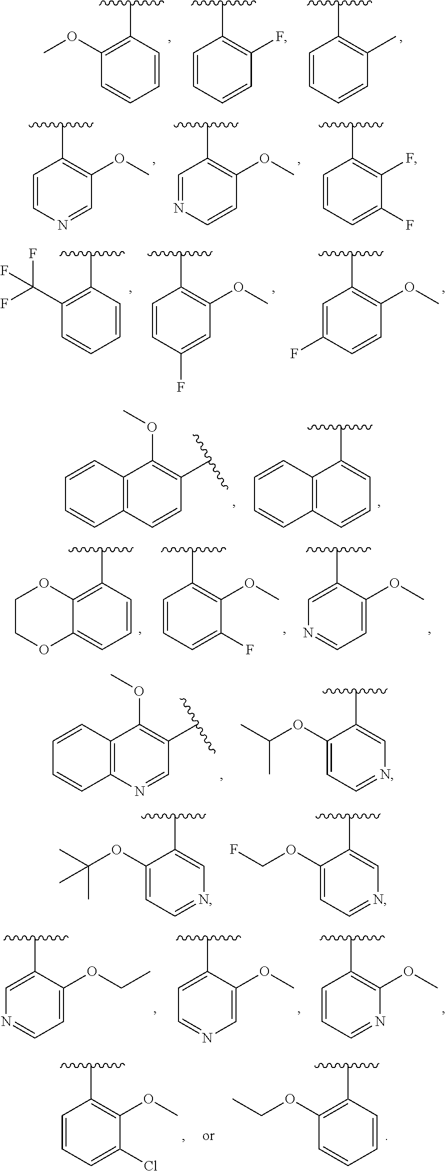

In some embodiments, R^{w} is —CH₂CH₂CH₃,

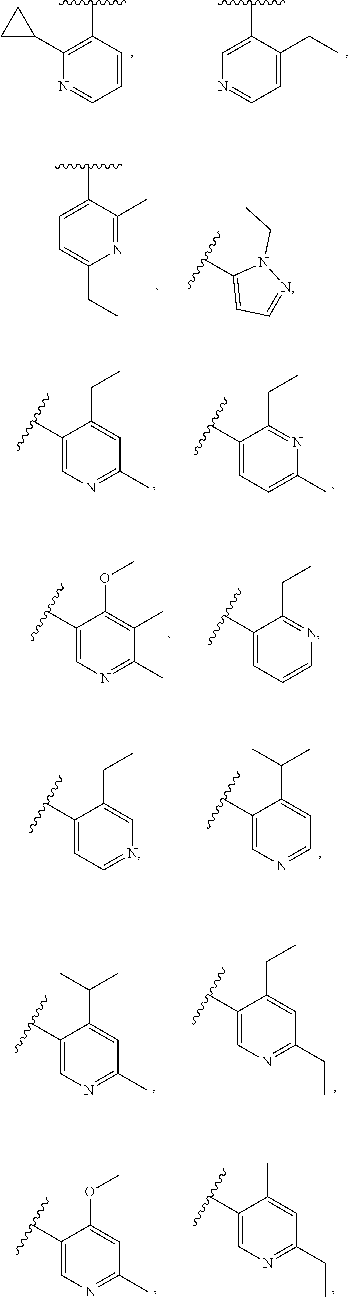

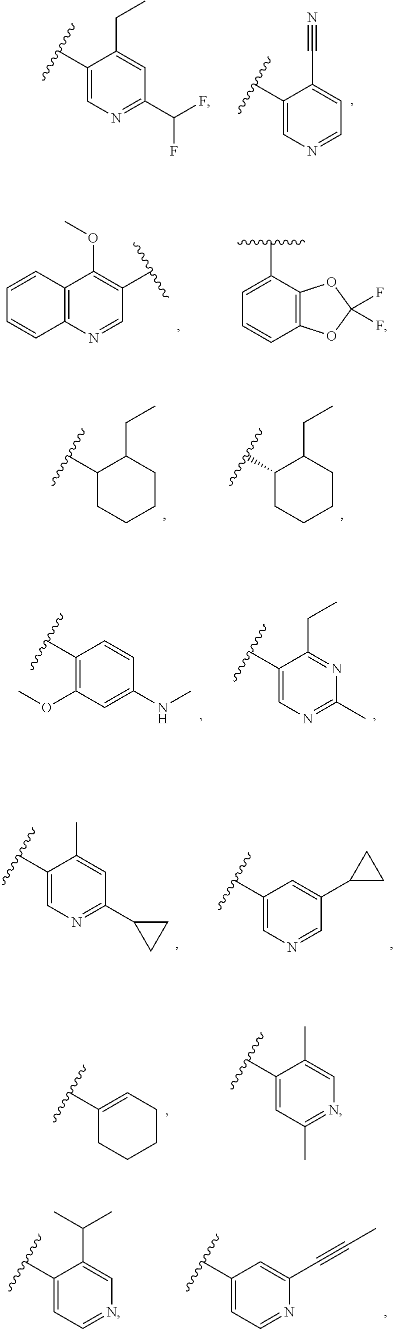

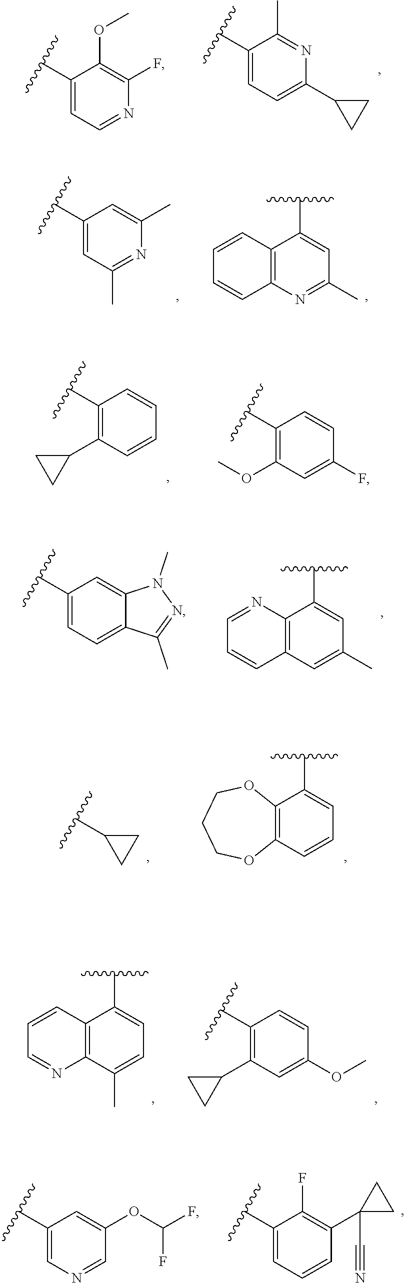

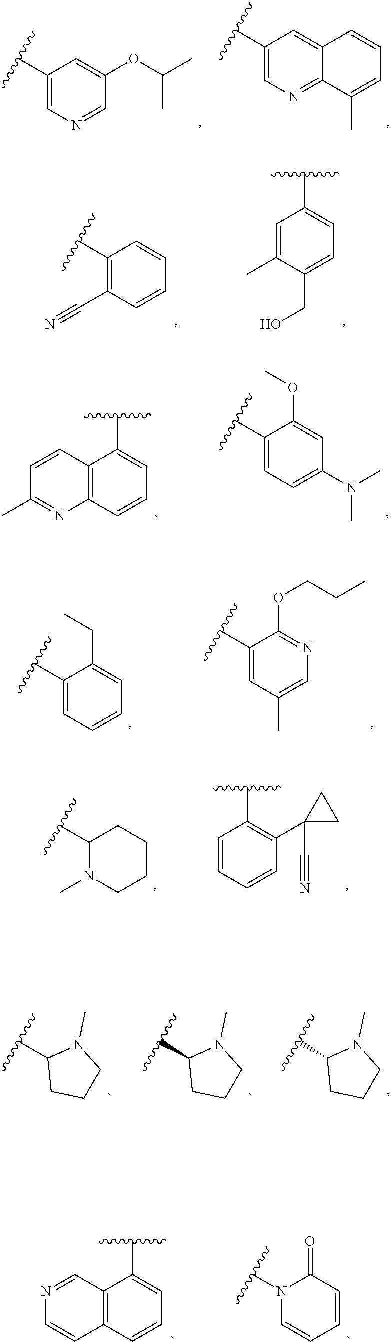

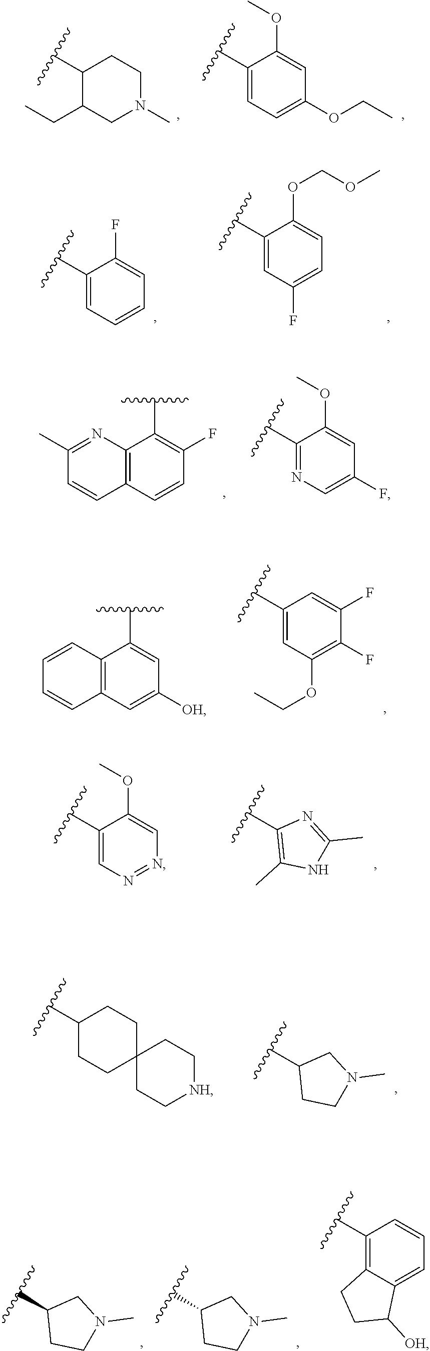

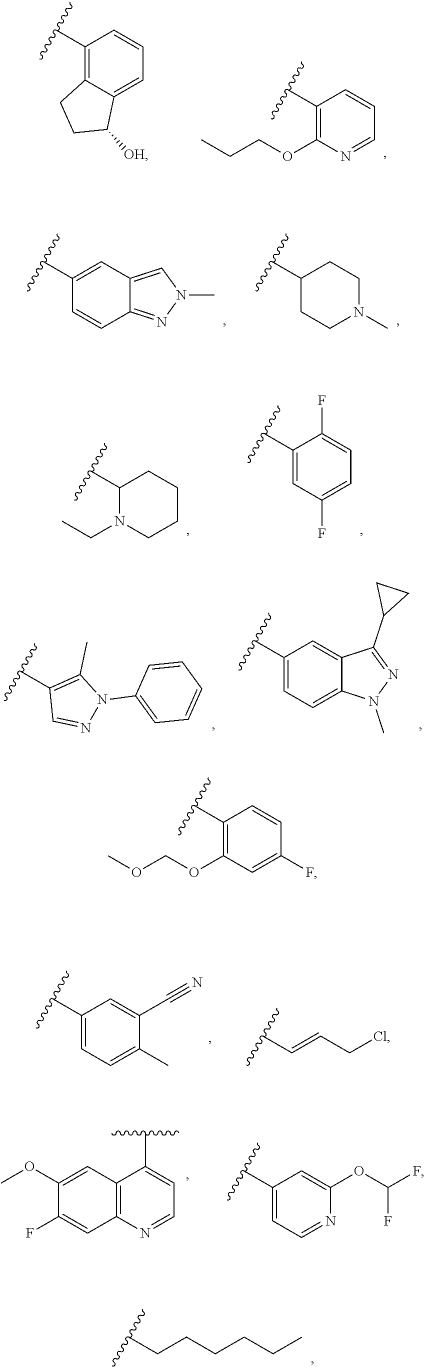

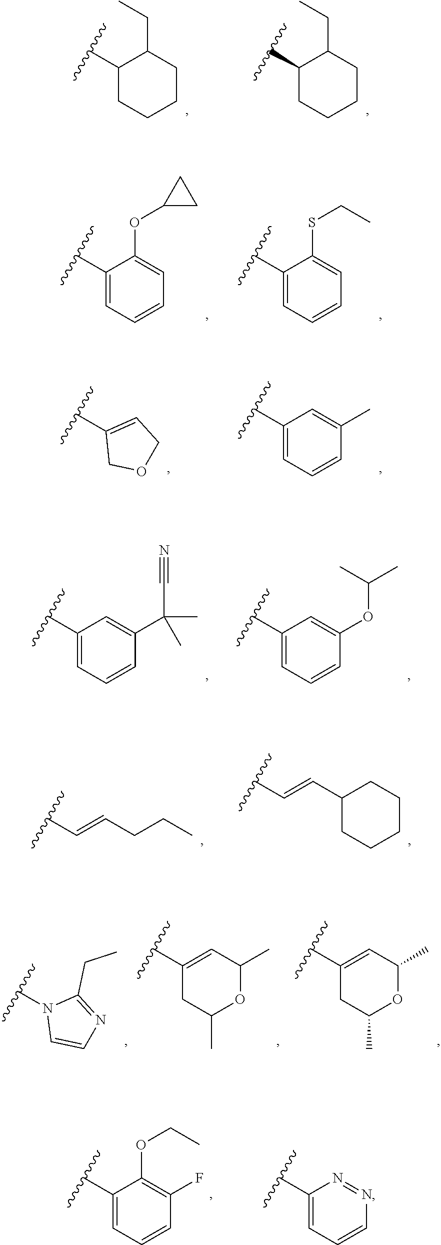

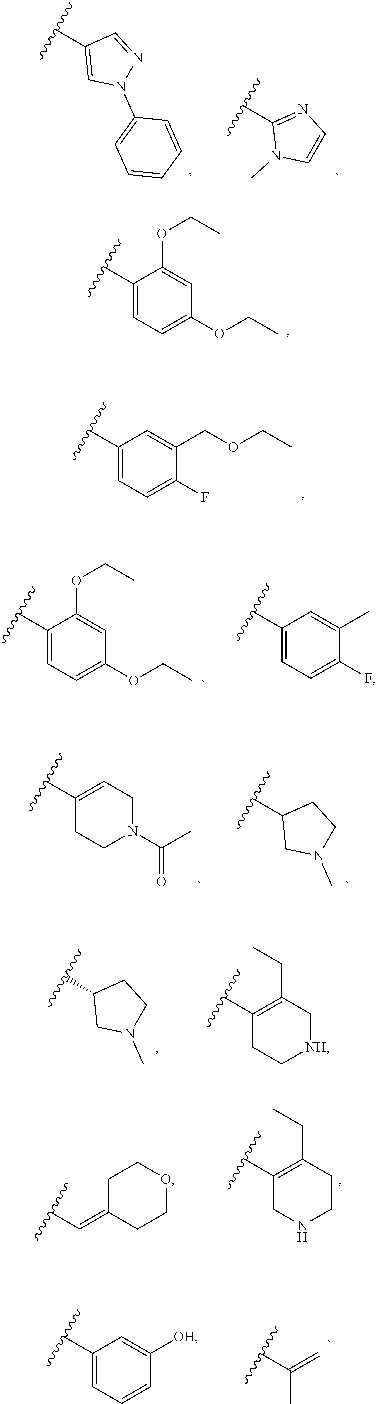

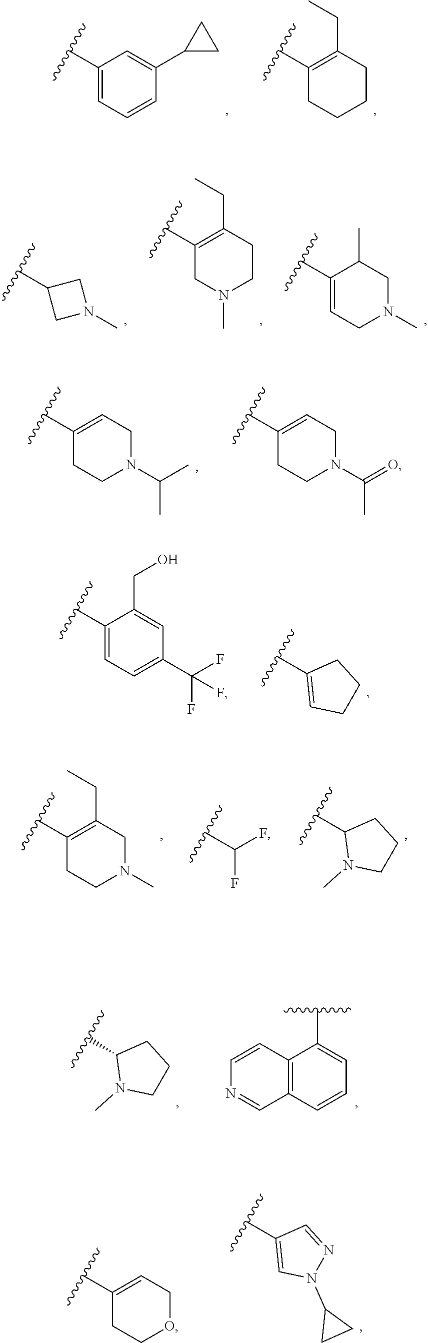

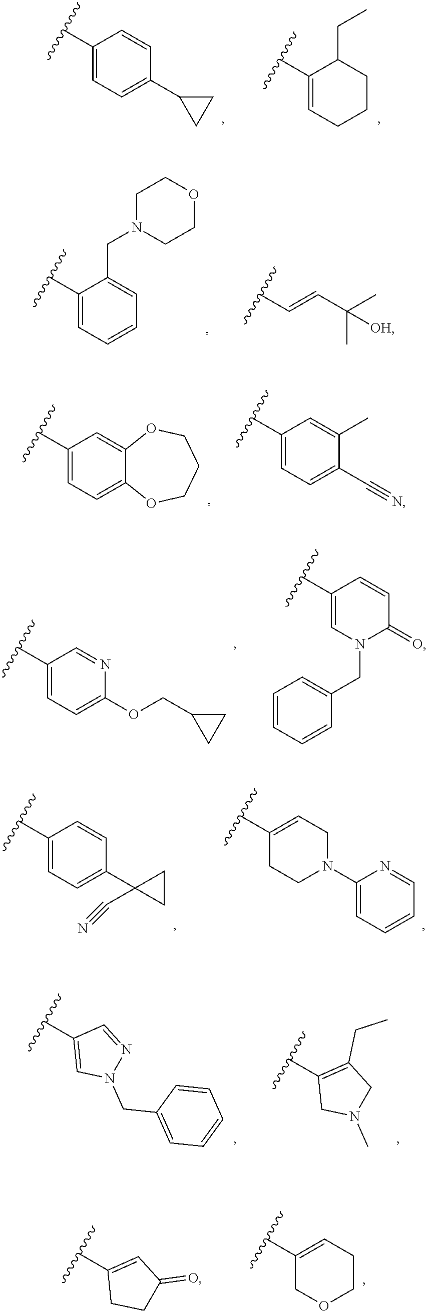

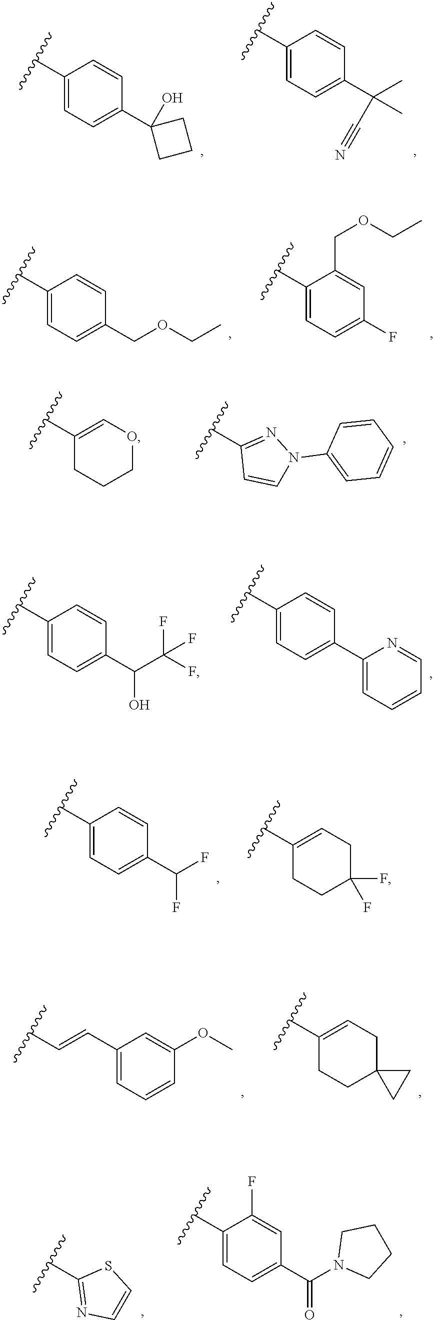

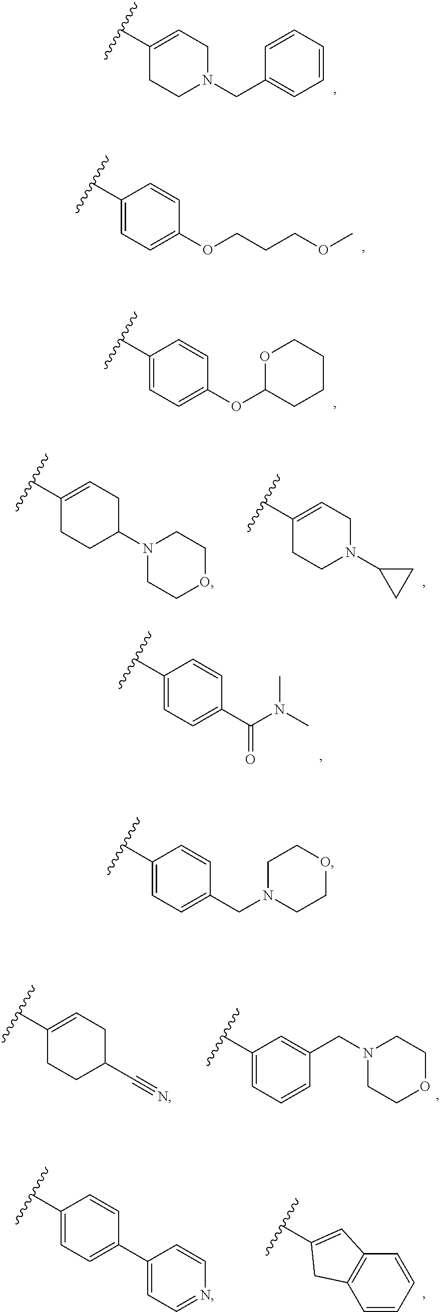

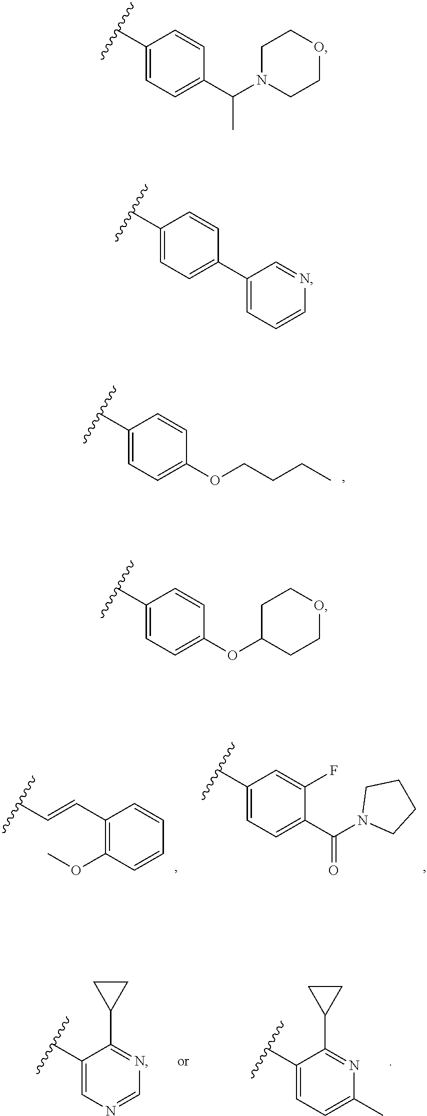

In some embodiments, R^{w} is

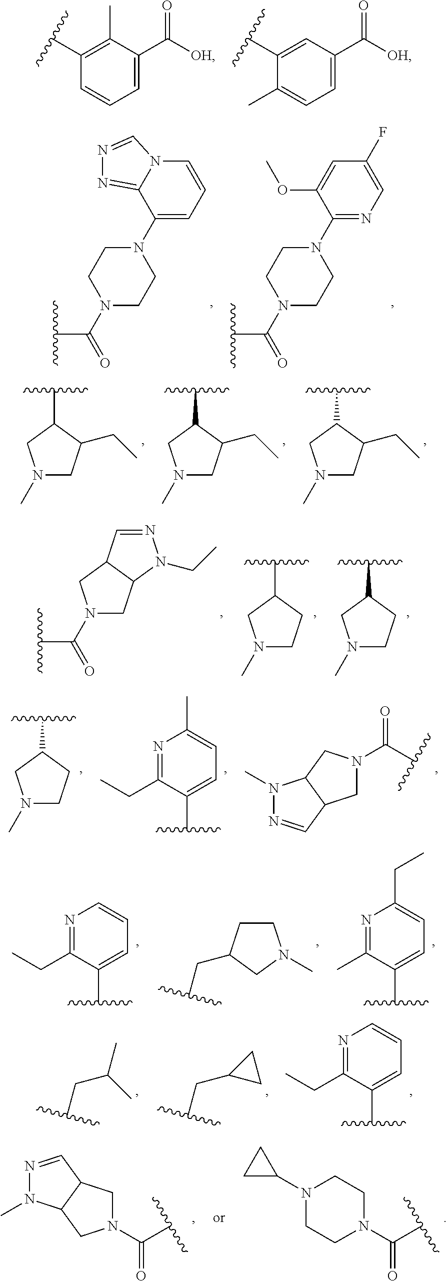

In some embodiments, R^{w} is —C(O)NH₂. In some embodiments, R^{w} is —C(O)NR₂, wherein each R is of R^{w} is independently hydrogen or an optionally substituted C₁₋₆ aliphatic. In some embodiments, R^{w} is —C(O)NR₂, wherein each R is of R^{w} is independently hydrogen or an optionally substituted phenyl. In some embodiments, R^{w} is —C(O)NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —C(O)NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is

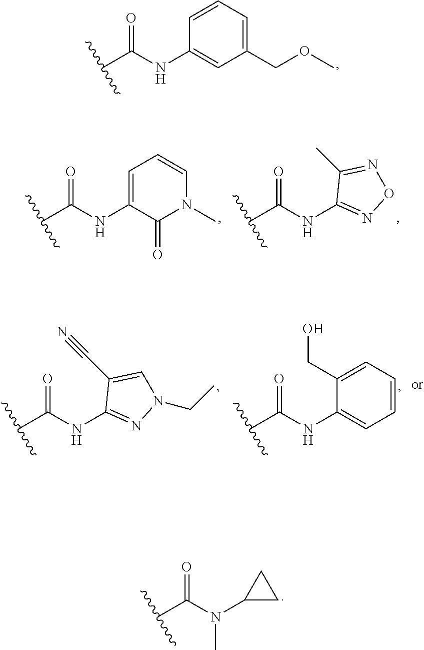

In some embodiments, each R^{x} is independently selected from R^{A}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, R^{x} is —CO₂Me, or —CH₂OH.

In some embodiments, R^{x} is —C(O)R or —C(O)OR. In some embodiments, R^{x} is —C(O)R. In some embodiments, R^{x} is —C(O)R, wherein R of R^{x} is an optionally substituted C₁₋₆ aliphatic. In some embodiments, R^{x} is —C(O)R, wherein R of R^{x} is C₁₋₆ aliphatic optionally substituted with —C(O)OR^{◯}, —OR^{◯}, halogen (e.g., fluoro), or ═O. In some embodiments, R^{x} is —C(O)OR. In some embodiments, R^{x} is —C(O)OR, wherein R of R^{x} is an optionally substituted C₁₋₆ aliphatic.

In some embodiments, R^{x} is

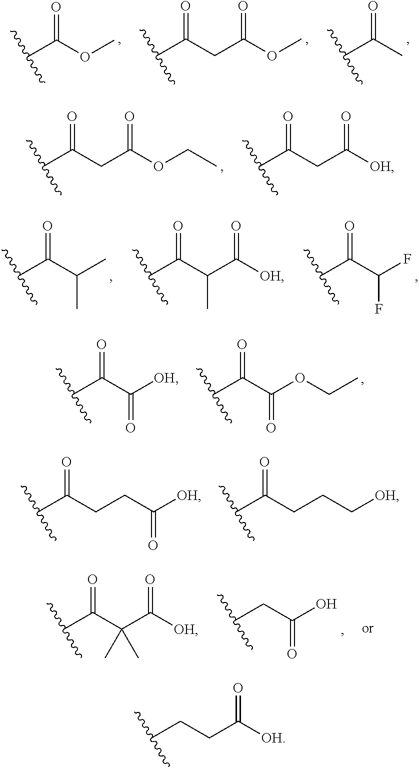

In some embodiments, R^{x} is optionally substituted C₁₋₆ aliphatic. In some embodiments, R^{x} is C₁₋₆ aliphatic optionally substituted with —OR^{◯}, wherein R^{◯} is hydrogen or C₁₋₆ aliphatic.

In some embodiments, R^{x} is —CO₂Me, —CH₂OH, —C(O)Me, —C(O)Et, —C(O)iPr, —C(O)cyclopropyl, —C(O)oxetanyl, —C(O)tetrahydropyranyl, or pyridyl.

In some embodiments, R^{x} is fluoro. In some embodiments, R^{x} is —S(O)₂CH₃. In some embodiments, R^{x} is —C(O)N(CH₃)₂.

In some embodiments, R^{x} is —C(O)R, wherein R of R^{x} is optionally substituted C₁₋₆ aliphatic. In some embodiments, R^{x} is —C(O)R, wherein R of R^{x} is C₁₋₆ aliphatic. In some embodiments, R^{x} is —C(O)R, wherein R of R^{x} is methyl, ethyl, n-propyl, or isopropyl. In some embodiments, R^{x} is —C(O)R, wherein R of R^{x} is C₁₋₆ aliphatic substituted with —OR^{◯} or —C(O)OR^{◯}, wherein R^{◯} is hydrogen or C₁₋₆ aliphatic. In some embodiments, R^{x} is —C(O)R, wherein R of R^{x} is C₁₋₆ aliphatic substituted with —OR^{◯}, wherein R^{◯} is hydrogen or C₁₋₆ aliphatic. In some embodiments, R^{x} is —C(O)R, wherein R of R^{x} is C₁₋₆ aliphatic substituted with —C(O)OR^{◯}, wherein R^{◯} is hydrogen or C₁₋₆ aliphatic.

In some embodiments, R^{x} is

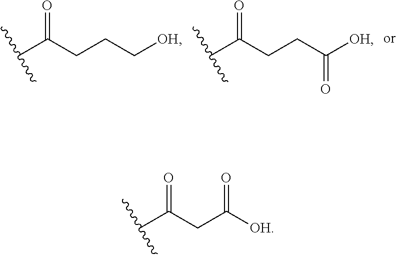 [SMILES: *C(=O)CCCO ; *C(=O)CCC(=O)O ; "or" ; *C(=O)CC(=O)O]

In some embodiments, each R^{y} is independently selected from R^{A}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, R^{y} is fluoro, chloro, bromo, iodo, methyl, ethyl, cyclopropyl, —CF₃, —CN, CH₂O, —CO₂H, —CO₂Me, —CO₂tBu, —C(O)Me, —NH₂, —NHMe, —NHAc, —NHC(O)Et, —OH, —OMe, —OCH₂CH₂NH₂, —CH₂OH, —CH₂OMe, —CH₂NHMe, —CH₂NHAc, —CH₂SO₂Me, —SO₂Me, —SO₂NH₂, —SO₂NHMe,

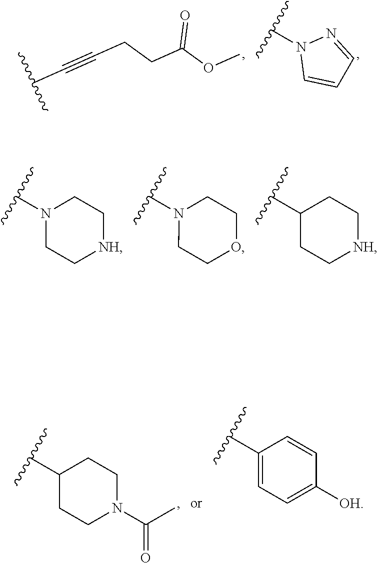

In some embodiments, R^{y} is —C(O)H.

In some embodiment, R^{w}, R^{x}, and R^{y} are as depicted in the compounds of Table 1A, below.

As described above and defined herein, each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{A} is an optionally substituted C₁₋₆ aliphatic. In some embodiments, R^{A} is an optionally substituted phenyl. In some embodiments, R^{A} is an optionally substituted 3-7 membered saturated or partially unsaturated carbocyclic. In some embodiments, R^{A} is an optionally substituted 3-7 membered saturated or partially unsaturated heterocyclic ring having 1-2 heteroatoms independently selected from nitrogen, oxygen, and sulfur. In some embodiments, R^{A} is an optionally substituted 5-6 membered heteroaryl ring having 1-4 heteroatoms independently selected from nitrogen, oxygen, and sulfur.

In some embodiments, R^{A} is C₁₋₆ alkyl (e.g., methyl, ethyl, isopropyl). In some embodiments, R^{A} is C₁₋₆ haloalkyl (e.g., —CF₃, —CHF₂).

In some embodiment, R^{A} is as depicted in the compounds of Table 1A, below.

As described above and defined herein, each R^{B} is independently -L^{B}-Cy^{B1}-H or -L^{B}-Cy^{B1}-Cy^{B2}.

In some embodiments, R^{B} is -L^{B}-Cy^{B1}-H. In some embodiments, R^{B} is -L^{B}-Cy^{B1}-Cy^{B2}.

In some embodiments, R^{B} is

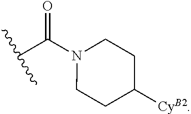

In some embodiments, R^{B} is

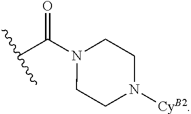

In some embodiments, R^{B} is

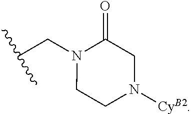

In some embodiments, R^{B} is

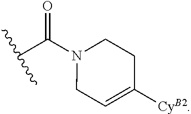

In some embodiments, R^{B} is

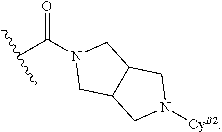

In some embodiments, R^{B} is

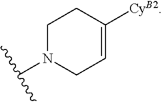

In some embodiments, R^{B} is

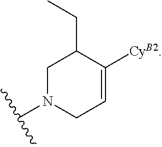

In some embodiments, R^{B} is,

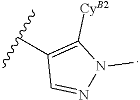

In some embodiments, R^{B} is

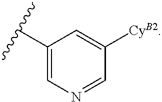

In some embodiments, R^{B} is

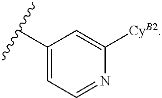

In some embodiments, R^{B} is

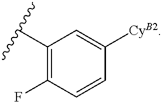

In some embodiments, R^{B} is

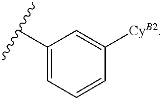

In some embodiments, R^{B} is

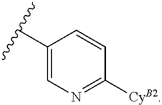

In some embodiments, R^{B} is

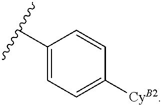

In some embodiments, R^{B} is as depicted in the compounds of Table 1A, below.

In some embodiments, R^{B} is

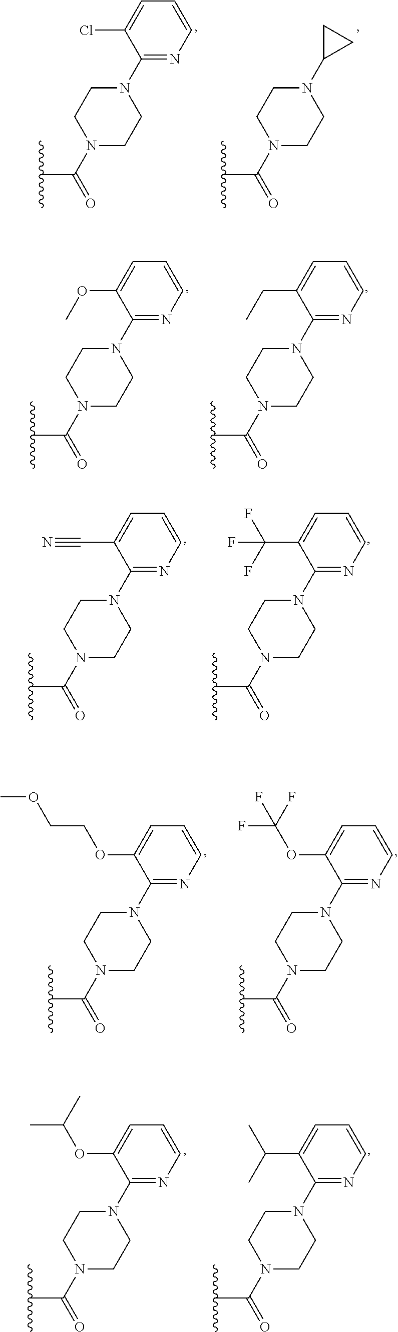

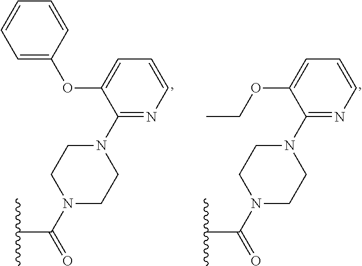 [SMILES: *C(=O)N1CCN(c2ncccc2Oc2ccccc2)CC1 ; *C(=O)N1CCN(c2ncccc2OCC)CC1]

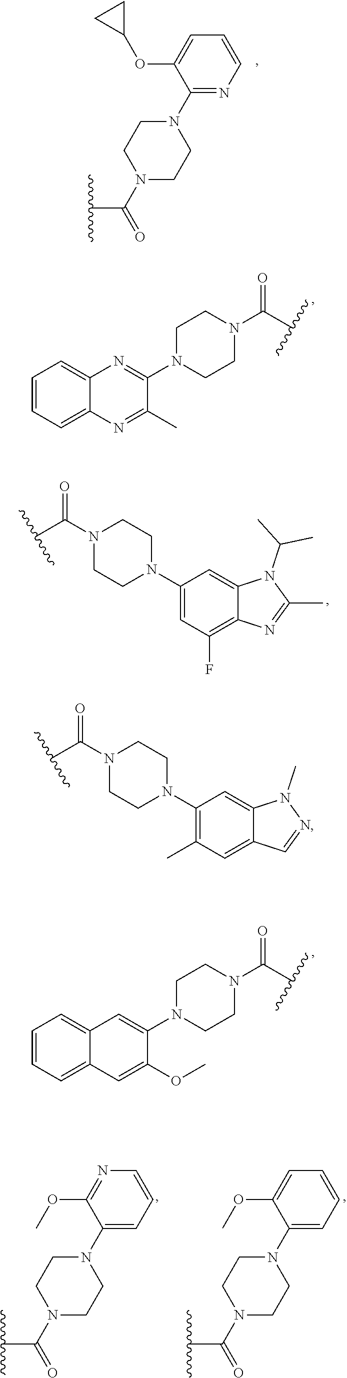

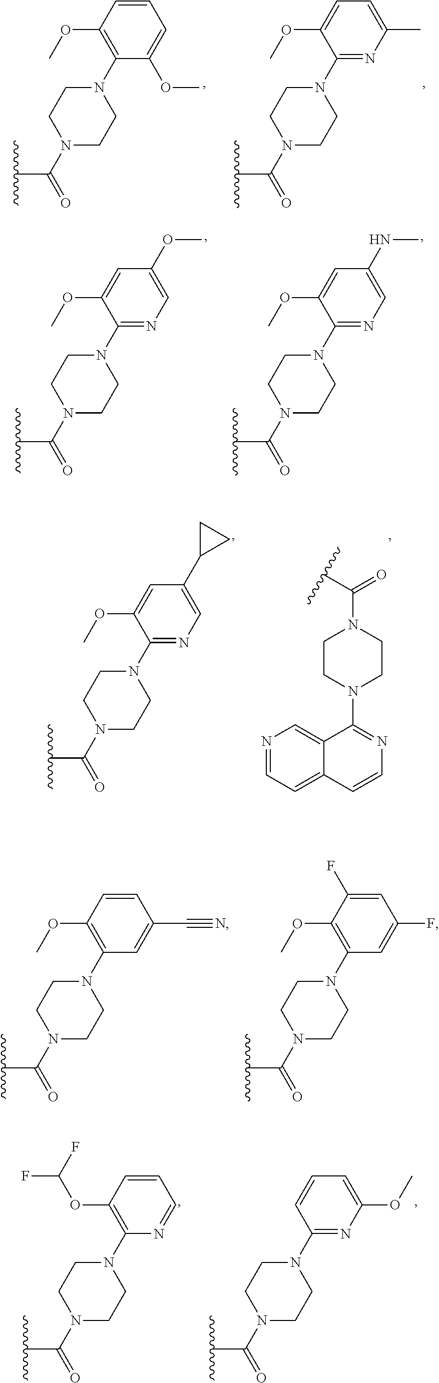

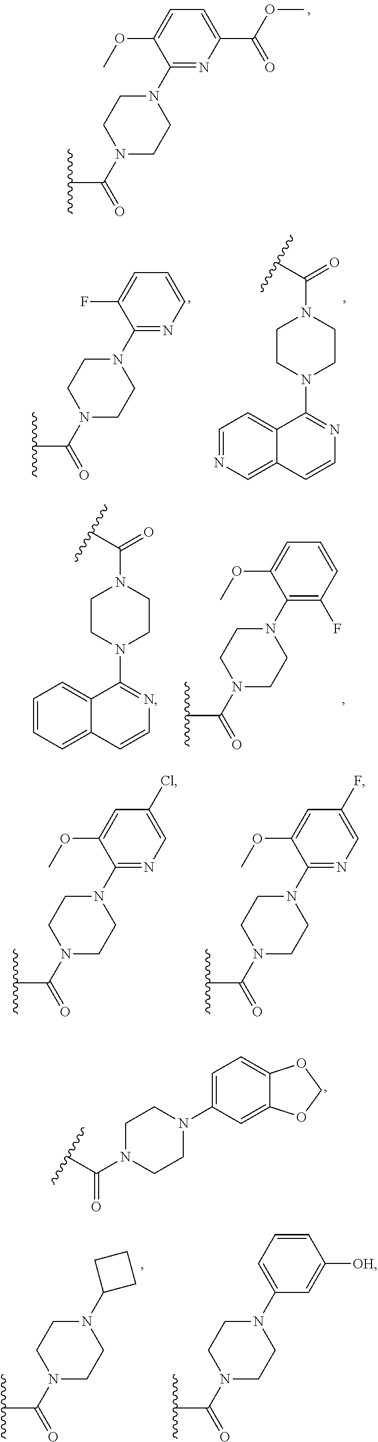

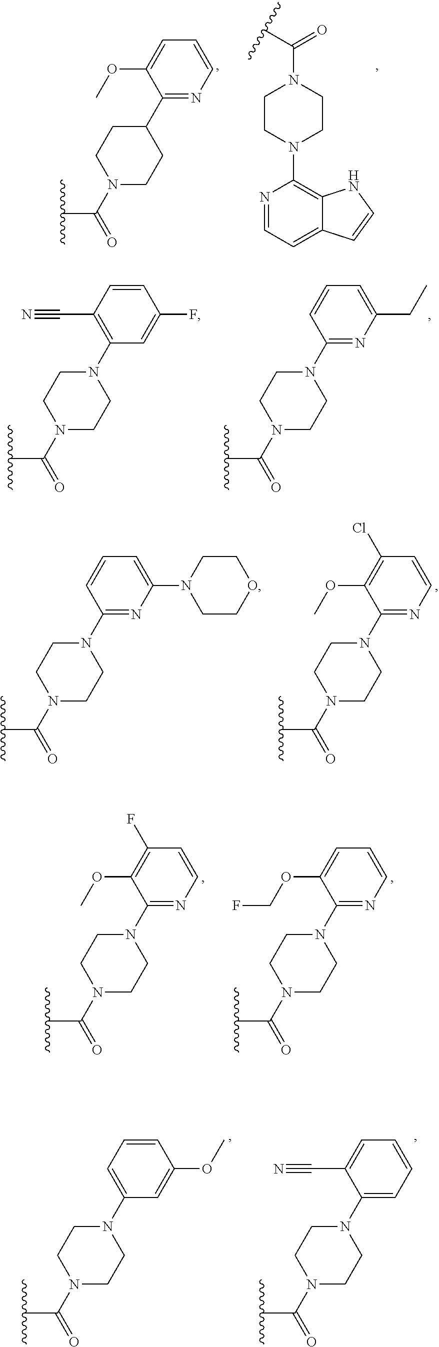

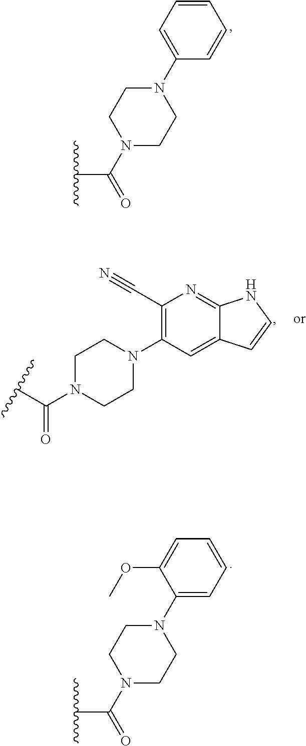 [SMILES: *C(=O)N1CCN(c2ccccc2)CC1 ; *C(=O)N1CCN(c2cc3cc[nH]c3nc2C#N)CC1 ; *C(=O)N1CCN(c2ccccc2OC)CC1]

In some embodiments, R^{B} is

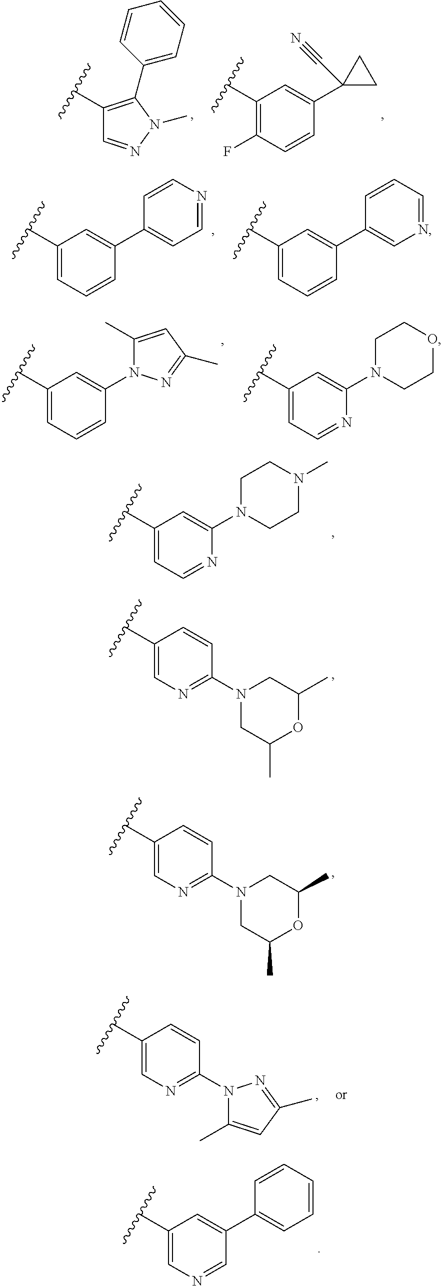

In some embodiments, R^{B} is

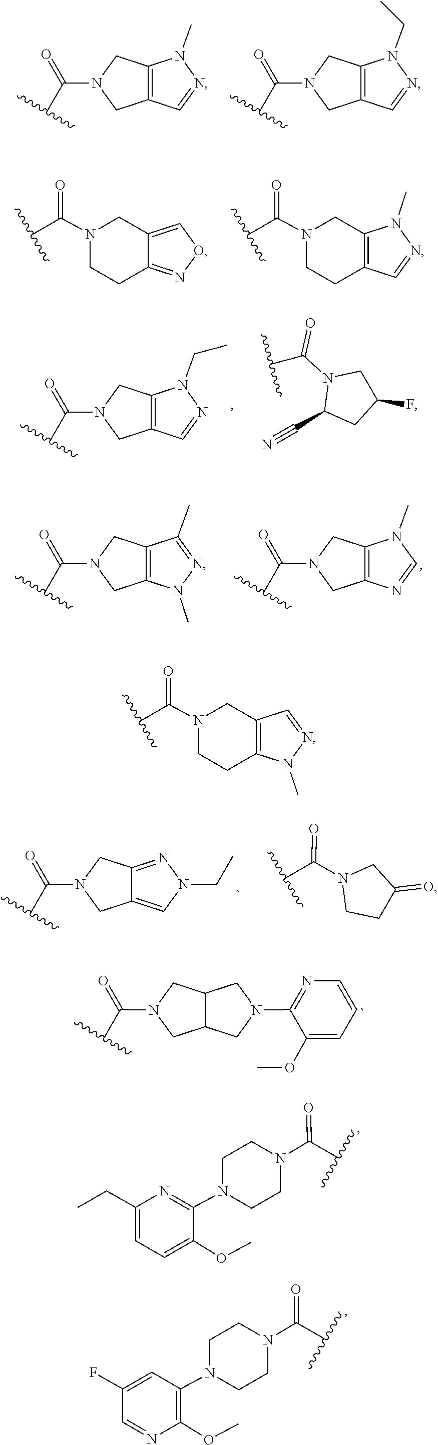

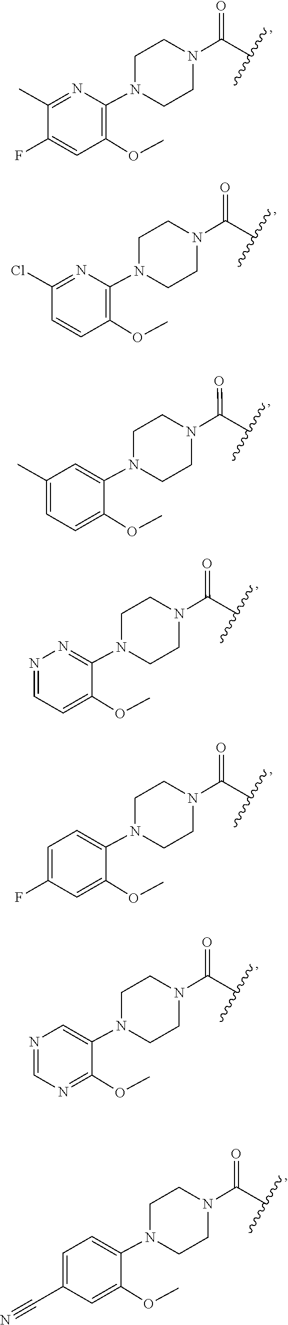

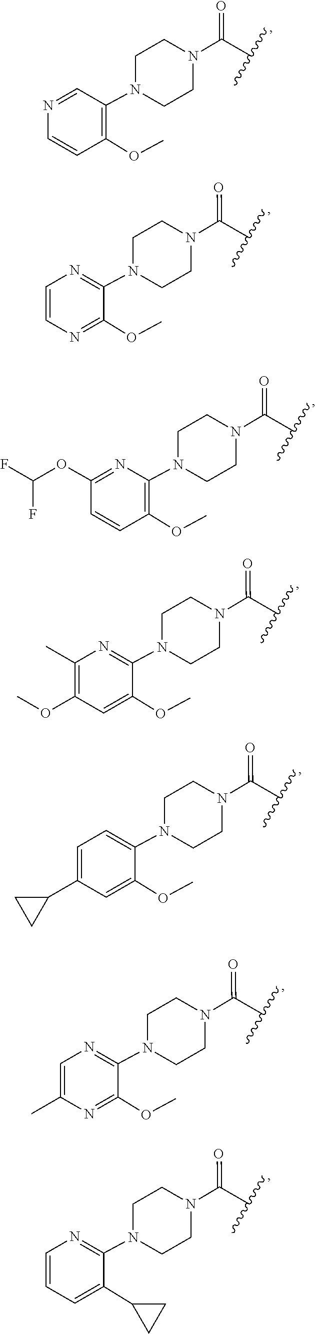

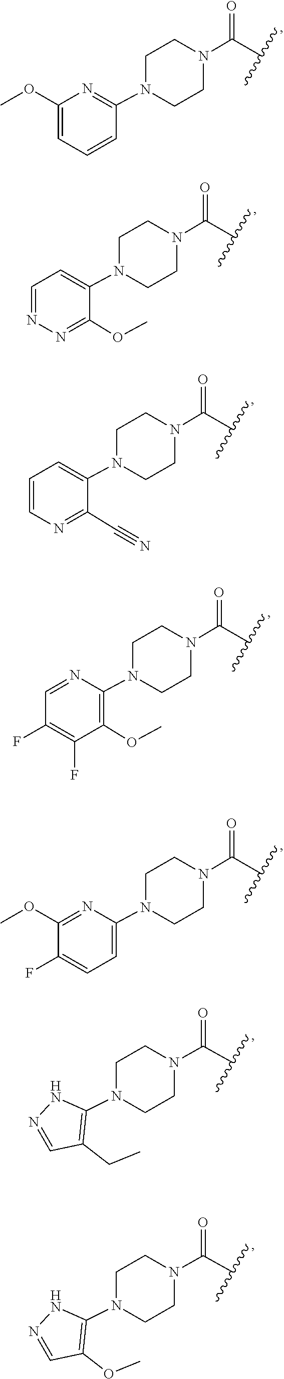

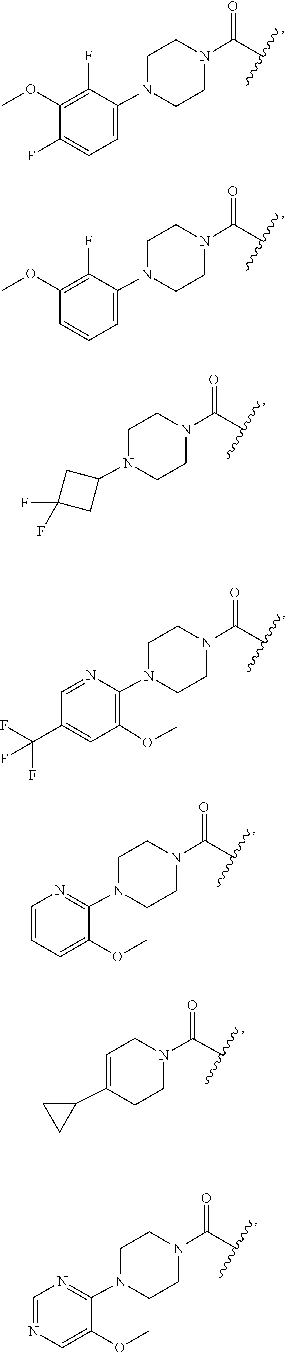

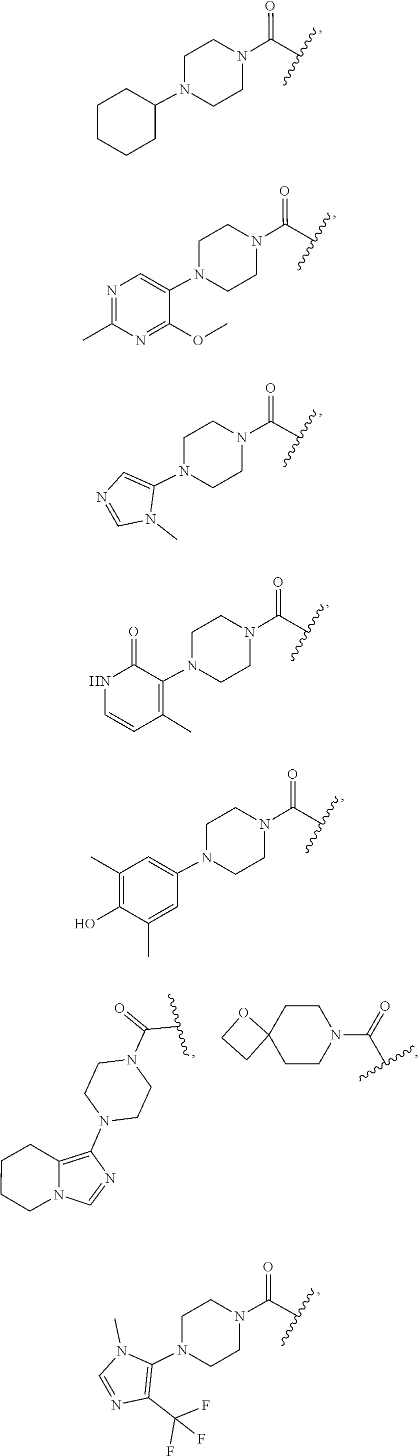

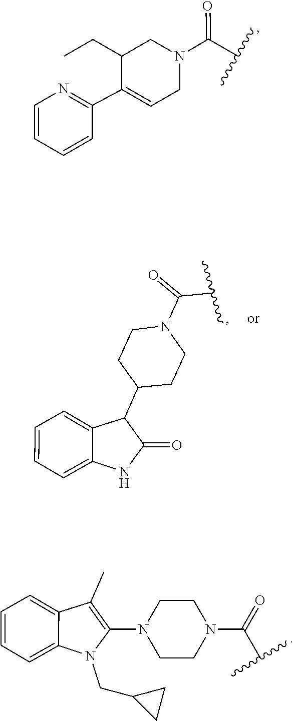 [SMILES: *C(=O)N1CC=C(c2ccccn2)C(CC)C1 ; *C(=O)N1CCC(C2C(=O)Nc3ccccc32)CC1 ; *C(=O)N1CCN(c2c(C)c3ccccc3n2CC2CC2)CC1]

As described above and defined herein, each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—.

In some embodiments, each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂— —S(O)(NR)— or —CR═CR—.

In some embodiments, L^{B} is a covalent bond. In some embodiments, L^{B} is a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂— —S(O)(NR)— or —CR═CR—. In some embodiments, L^{B} is a C₁₋₃ bivalent straight or branched saturated or unsaturated hydrocarbon chain wherein 1-2 methylene units of the chain are independently and optionally replaced with —O—, —C(O)—, —NR—, —S—, —S(O)—, or —S(O)₂—. In some embodiments, L^{B} is a C₁₋₃ bivalent straight or branched saturated or unsaturated hydrocarbon chain wherein 1-2 methylene units of the chain are independently and optionally replaced with —O—, —C(O)—, or —NR—. In some embodiments, L^{B} is a C₁₋₃ bivalent straight or branched saturated or unsaturated hydrocarbon chain wherein 1 methylene unit of the chain is optionally replaced with —O—, —C(O)—, —NR—, —S—, —S(O)—, or —S(O)₂—. In some embodiments, L^{B} is a C₁₋₃ bivalent straight or branched saturated or unsaturated hydrocarbon chain wherein 1 methylene unit of the chain is optionally replaced with —O—, —C(O)—, or —NR—. In some embodiments, L^{B} is a C₁₋₃ bivalent straight or branched saturated or unsaturated hydrocarbon chain wherein 1 methylene unit of the chain is replaced with —C(O)—. In some embodiments, L^{B} is —C(O)—.

In some embodiments, L^{B} is a C₁₋₃ bivalent straight or branched saturated or unsaturated hydrocarbon chain. In some embodiments, L^{B} is —CH₂—.

In some embodiments, L^{B} is —C(O)—, —C(S)—, —C(NR)R—, —S(O)—, —S(O)₂— or —S(O)(NR)—. In some embodiments, L^{B} is —C(O)—, —C(S)—, or —C(NR)R—. In some embodiments, L^{B} is —C(O)—, —S(O)—, —S(O)₂— or —S(O)(NR)—. In some embodiments, L^{B} is —C(S)—. In some embodiments, L^{B} is —C(NR)R—. In some embodiments, L^{B} is —S(O)—. In some embodiments, L^{B} is —S(O)₂—. In some embodiments, L^{B} is —S(O)(NR)—.

As described above and defined herein, each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, a 5-10 membered monocyclic or bicyclic arylenyl or heteroarylenyl having 1-4 heteroatoms independently selected from nitrogen, oxygen, and sulfur; or a 5-15 membered saturated or partially saturated monocyclic, bicyclic, or tricyclic carbocyclylenyl, heterocyclylenyl, arylenyl, or heteroarylenyl having 1-4 heteroatoms independently selected from nitrogen, oxygen, and sulfur.

In some embodiments, each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur.

It will be appreciated that all embodiments to Cy^{B1} may refer to a terminal ring (or otherwise optionally substituted ring) in structures with -L^{B}-Cy^{B1} (i.e., when R^{B} is -L^{B}-Cy^{B1}-H), or a ring further connected to Cy^{B2} as in structures -L^{B}-Cy^{B1}-Cy^{B2}, regardless of how presented. By way of example, an embodiment to Cy^{B1} is phenylenyl, refers to phenylenyl in -L^{B}-Cy^{B1}-Cy^{B2}, and phenyl in -L^{B}-Cy^{B1}-H. Similarly, an embodiment to Cy^{B1} is

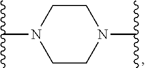 [SMILES: *N1CCN(*)CC1]

refers to

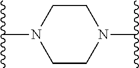 [SMILES: *N1CCN(*)CC1]

in -L^{B}-Cy^{B1}-Cy^{B2}, and

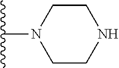 [SMILES: *N1CCNCC1]

in -L^{B} Cy^{B1}-H. Similarly, an embodiment to Cy^{B1} is

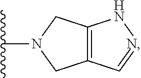 [SMILES: *N1Cc2cn[nH]c2C1]

refers to

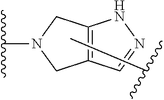

in -L^{B}-Cy^{B1}-Cy^{B2},

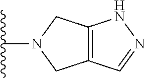 [SMILES: *N1Cc2cn[nH]c2C1]

and in -L^{B}-Cy^{B1}-H.

In some embodiments, each Cy^{B1} is independently an optionally substituted ring selected from phenylenyl, a 3-7 membered saturated or partially unsaturated carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur.

In some embodiments, Cy^{B1} is optionally substituted phenylenyl. In some embodiments, Cy^{B1} is phenylenyl.

In some embodiments, Cy^{B1} is optionally substituted 3-7 membered saturated or partially unsaturated carbocyclylenyl or heterocyclylenyl having 1-4 heteroatoms independently selected from nitrogen, oxygen, and sulfur. In some embodiments, Cy^{B1} is optionally substituted 5-10 membered monocyclic or bicyclic arylenyl or heteroarylenyl having 1-4 heteroatoms independently selected from nitrogen, oxygen, and sulfur.

In some embodiments, Cy^{B1} is optionally substituted 3-7 membered saturated or partially unsaturated heterocyclylenyl having 1-4 heteroatoms independently selected from nitrogen, oxygen, and sulfur. In some embodiments, Cy^{B1} is optionally substituted 6-membered saturated or partially unsaturated heterocyclylenyl having 1-4 heteroatoms independently selected from nitrogen, oxygen, and sulfur. In some embodiments, Cy^{B1} is optionally substituted 6-membered saturated or partially unsaturated heterocyclylenyl having 1-2 heteroatoms independently selected from nitrogen, oxygen, and sulfur. In some embodiments, Cy^{B1} is optionally substituted 6-membered saturated or partially unsaturated heterocyclylenyl having 1-2 nitrogen heteroatoms. In some embodiments, Cy^{B1} is optionally substituted piperadinylenyl or piperazinylenyl.

In some embodiments, Cy^{B1} is a 3-7 membered saturated or partially unsaturated heterocyclylenyl having 1-4 heteroatoms independently selected from nitrogen, oxygen, and sulfur. In some embodiments, Cy^{B1} is a 6-membered saturated or partially unsaturated heterocyclylenyl having 1-4 heteroatoms independently selected from nitrogen, oxygen, and sulfur. In some embodiments, Cy^{B1} is a 6-membered saturated or partially unsaturated heterocyclylenyl having 1-2 heteroatoms independently selected from nitrogen, oxygen, and sulfur. In some embodiments, Cy^{B1} is a 6-membered saturated or partially unsaturated heterocyclylenyl having 1-2 nitrogen heteroatoms. In some embodiments, Cy^{B1} is a piperadinylenyl or piperazinylenyl. In some embodiments, Cy^{B1} is

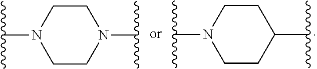

In some embodiments, Cy^{B1} is an optionally substituted piperidinonyl or piperazinonyl. In some embodiments, Cy^{B1} is an optionally substituted dihydropyridinyl. In some embodiments, Cy^{B1} is an optionally substituted thiomorpholinyl.

In some embodiments, Cy^{B1} is optionally substituted morpholinyl.

In some embodiments, Cy^{B1} is a 3-10 membered saturated or partially unsaturated monocyclic or bicyclic heterocyclylenyl having 1-4 heteroatoms independently selected from nitrogen, oxygen, and sulfur.

In some embodiments, Cy^{B1} is an optionally substituted 8-10 membered saturated or partially unsaturated bicyclic heterocyclylenyl having 1-4 heteroatoms independently selected from nitrogen, oxygen, and sulfur. In some embodiments, Cy^{B1} is an optionally substituted 8-membered saturated or partially unsaturated heterocyclylenyl having 1-4 heteroatoms independently selected from nitrogen, oxygen, and sulfur. In some embodiments, Cy^{B1} is an optionally substituted 8-membered saturated or partially unsaturated heterocyclylenyl having 1-2 heteroatoms independently selected from nitrogen, oxygen, and sulfur. In some embodiments, Cy^{B1} is an optionally substituted 8-membered saturated or partially unsaturated heterocyclylenyl having 1-2 nitrogen heteroatoms. In some embodiments, Cy^{B1} is optionally substituted

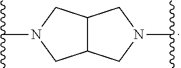 [SMILES: *N1CC2CN(*)CC2C1 ; "."]

In some embodiments, Cy^{B1} is an optionally substituted

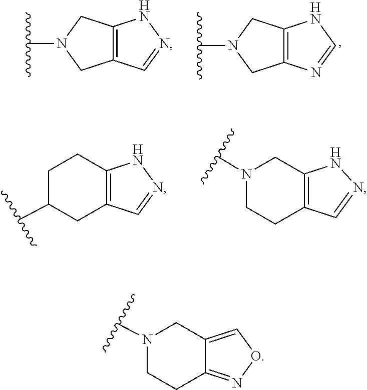

In some embodiments, Cy^{B1} is optionally substituted indazolyl. In some embodiments, Cy^{B1} is optionally substituted pyrrolo[2,3-b]pyridine.

In some embodiments, Cy^{B1} is an optionally substituted 5-6 membered monocyclic heteroarylenyl having 1-4 heteroatoms independently selected from nitrogen, oxygen, and sulfur. In some embodiments, Cy^{B1} is an optionally substituted 6 membered monocyclic heteroarylenyl having 1-4 heteroatoms independently selected from nitrogen, oxygen, and sulfur. In some embodiments, Cy^{B1} is an optionally substituted pyridinonyl, pyrazinonyl, or pyrimidinoyl.

In some embodiments, Cy^{B1} is optionally substituted furanyl. In some embodiments, Cy^{B1} is optionally substituted isoxazolyl. In some embodiments, Cy^{B1} is optionally substituted pyrrolyl. In some embodiments, Cy^{B1} is optionally substituted pyridinonyl. In some embodiments, Cy^{B1} is optionally substituted pyridazinyl. In some embodiments, Cy^{B1} is an optionally substituted 5-15 membered saturated or partially saturated monocyclic, bicyclic, or tricyclic carbocyclylenyl, heterocyclylenyl, arylenyl, or heteroarylenyl having 1-4 heteroatoms independently selected from nitrogen, oxygen, and sulfur.

In some embodiments, Cy^{B1} is an optionally substituted 10-15 membered saturated or partially saturated tricyclic carbocyclylenyl, heterocyclylenyl, arylenyl, or heteroarylenyl having 1-4 heteroatoms independently selected from nitrogen, oxygen, and sulfur. In some embodiments, Cy^{B1} is an optionally substituted 10-15 membered saturated or partially saturated tricyclic heterocyclylenyl having 1-4 heteroatoms independently selected from nitrogen, oxygen, and sulfur. In some embodiments, Cy^{B1} is an optionally substituted 10-15 membered heteroarylenyl having 1-4 heteroatoms independently selected from nitrogen, oxygen, and sulfur.

In some embodiments, Cy^{B1} is a 13-membered saturated or partially saturated tricyclic carbocyclylenyl, heterocyclylenyl, arylenyl, or heteroarylenyl having 1-4 heteroatoms independently selected from nitrogen, oxygen, and sulfur. In some embodiments, Cy^{B1} is a 15-membered saturated or partially saturated tricyclic carbocyclylenyl, heterocyclylenyl, arylenyl, or heteroarylenyl having 1-4 heteroatoms independently selected from nitrogen, oxygen, and sulfur.

As described above and defined herein, each Cy^{B2} is independently an optionally substituted ring selected from phenyl, a 3-10 membered saturated or partially unsaturated monocyclic or bicyclic carbocyclic or heterocyclic ring having 1-4 heteroatoms independently selected from nitrogen, oxygen, and sulfur, a 5-10 membered monocyclic or bicyclic aryl or heteroaryl having 1-4 heteroatoms independently selected from nitrogen, oxygen, and sulfur; or a 5-15 membered saturated or partially saturated monocyclic, bicyclic, or tricyclic carbocyclyl, heterocyclyl, aryl, or heteroaryl having 1-4 heteroatoms independently selected from nitrogen, oxygen, and sulfur.

In some embodiments, each Cy^{B2} is independently an optionally substituted ring selected from phenyl, a 3-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur.

In some embodiments, each Cy^{B2} is independently an optionally ring selected from phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur.

In some embodiments, Cy^{B2} is optionally substituted phenyl. In some embodiments, Cy^{B2} is phenyl. In some embodiments, Cy^{B2} is phenyl, optionally substituted with —CN, halogen, —R^{◯}, or —OR^{◯}, wherein R^{◯} is hydrogen or C₁₋₆ aliphatic.

In some embodiments, Cy^{B2} is optionally substituted 3-7 membered saturated or partially unsaturated carbocyclic or heterocyclic ring having 1-2 heteroatoms independently selected from nitrogen, oxygen, and sulfur.

In some embodiments, Cy^{B2} is optionally substituted 3-7 membered saturated or partially unsaturated carbocyclic. In some embodiments, Cy^{B2} is optionally substituted cyclopropyl. In some embodiments, Cy^{B2} is cyclopropyl. In some embodiments, C^{y} is optionally substituted cyclobutyl. In some embodiments, Cy^{B2} is cyclobutyl.

In some embodiments, Cy^{B2} is optionally substituted cyclopentyl. In some embodiments, Cy^{B2} is cyclopentyl. In some embodiments, Cy^{B2} is optionally substituted cyclohexyl. In some embodiments, Cy^{B2} is cyclohexyl.

In some embodiments, Cy^{B2} is optionally substituted 5-10 membered monocyclic or bicyclic aryl or heteroaryl with 1-4 heteroatoms independently selected from nitrogen, oxygen and sulfur.

In some embodiments, Cy^{B2} is optionally substituted 5-6 membered monocyclic heteroaryl with 1-4 heteroatoms independently selected from nitrogen, oxygen and sulfur. In some embodiments, Cy^{B2} is optionally substituted 5-6 membered monocyclic heteroaryl with 1-2 heteroatoms independently selected from nitrogen, oxygen and sulfur. In some embodiments, Cy^{B2} is optionally substituted pyridinyl. In some embodiments, Cy^{B2} is pyridinyl, optionally substituted with —CN, halogen, —R^{◯}, —OR^{◯}, —N(R^{◯})₂—C(O)OR^{◯}, wherein each R^{◯} is independently hydrogen; C₁₋₆ aliphatic, which may be optionally substituted with halogen, —(CH₂)₀₋₂OH, or —(CH₂)₀₋₂OR^{●}, where R^{●} is C₁₋₄ aliphatic; or a 5-6-membered saturated, partially unsaturated, or aryl ring having 0-4 heteroatoms independently selected from nitrogen, oxygen, or sulfur (e.g., phenyl or morpholinyl).

In some embodiments, Cy^{B2} is optionally substituted 6 membered monocyclic heteroaryl with 1-2 heteroatoms independently selected from nitrogen, oxygen and sulfur. In some embodiments, Cy^{B2} is optionally substituted pyridinyl, pyrimidinyl, pyridazinyl, pyrazinyl, or triazinyl.

In some embodiments, Cy^{B2} is optionally substituted optionally substituted pyridinonyl, pyrazinonyl, or pyrimidinoyl

In some embodiments, Cy^{B2} is optionally substituted 5 membered monocyclic heteroaryl with 1-2 heteroatoms independently selected from nitrogen, oxygen and sulfur. In some embodiments, Cy^{B2} is optionally substituted pyrazolyl, imidazolyl, or triazolyl.

In some embodiments, Cy^{B2} is optionally substituted 8-10 membered bicyclic aryl or heteroaryl with 1-4 heteroatoms independently selected from nitrogen, oxygen and sulfur.

In some embodiments, Cy^{B2} is optionally substituted 9-membered bicyclic heteroaryl with 1-4 heteroatoms independently selected from nitrogen, oxygen and sulfur. In some embodiments, Cy^{B2} is optionally substituted 9-membered bicyclic heteroaryl with 1-4 nitrogen heteroatoms. In some embodiments, Cy^{B2} is optionally substituted benzimidazolyl, indazolyl, or azaindolyl (e.g., pyrrolo[2,3-c]pyridinyl or pyrrolo[2,3-b]pyridinyl). In some embodiments, Cy^{B2} is benzimidazolyl indazolyl, or azaindolyl (e.g., pyrrolo[2,3-c]pyridinyl or pyrrolo[2,3-b]pyridinyl) optionally substituted with —CN, halogen or —R^{◯}, wherein R^{◯} is hydrogen or C₁₋₆ aliphatic. In some embodiments, Cy^{B2} is optionally substituted indolyl or azaindolyl.

In some embodiments, Cy^{B2} is optionally substituted [1,2,4]triazolo[4,3-a]pyridinyl.

In some embodiments, Cy^{B2} is optionally substituted 10-membered bicyclic aryl or heteroaryl with 1-4 heteroatoms independently selected from nitrogen, oxygen and sulfur. In some embodiments, Cy^{B2} is optionally substituted 10-membered bicyclic heteroaryl with 1-4 nitrogen heteroatoms. In some embodiments, Cy^{B2} is optionally substituted quinoxalinyl, isoquinolinyl, 2,6-naphthyridinyl, or 2,7-naphthyridinyl. In some embodiments, Cy^{B2} is quinoxalinyl, isoquinolinyl, 2,6-naphthyridinyl, or 2,7-naphthyridinyl, optionally substituted with —R^{◯}, wherein R^{◯} is hydrogen or C₁₋₆ aliphatic.

In some embodiments, Cy^{B2} is optionally substituted naphthalenyl. In some embodiments, Cy^{B2} is naphthalenyl, optionally substituted with —R^{◯} or —OR^{◯}, wherein R^{◯} is hydrogen or C₁₋₆ aliphatic.

In some embodiments, Cy^{B2} is optionally substituted benzo[d][1,3]dioxolyl.

In some embodiments, Cy^{B2} is

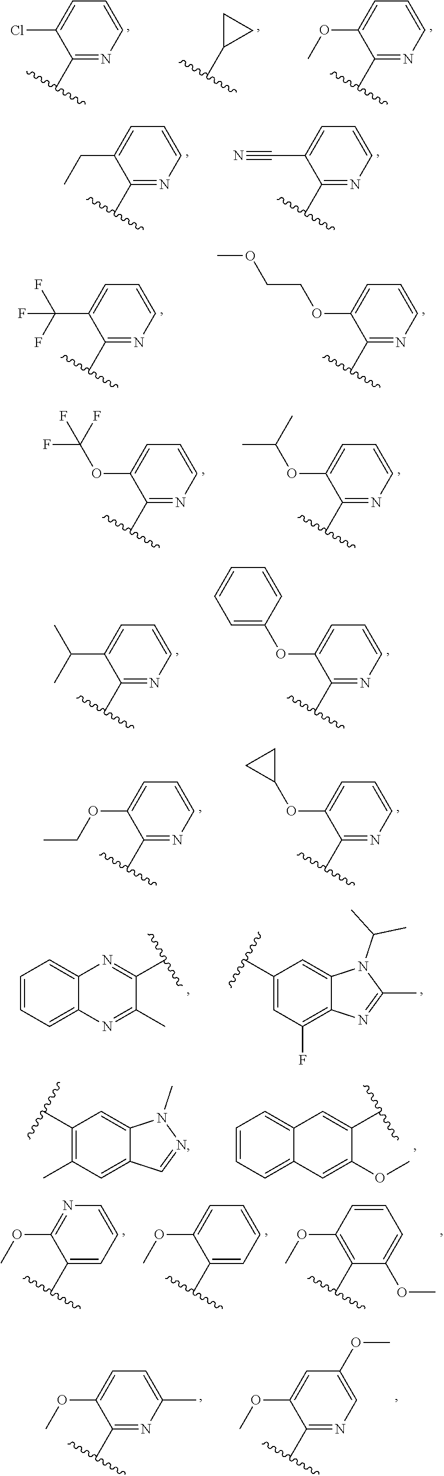

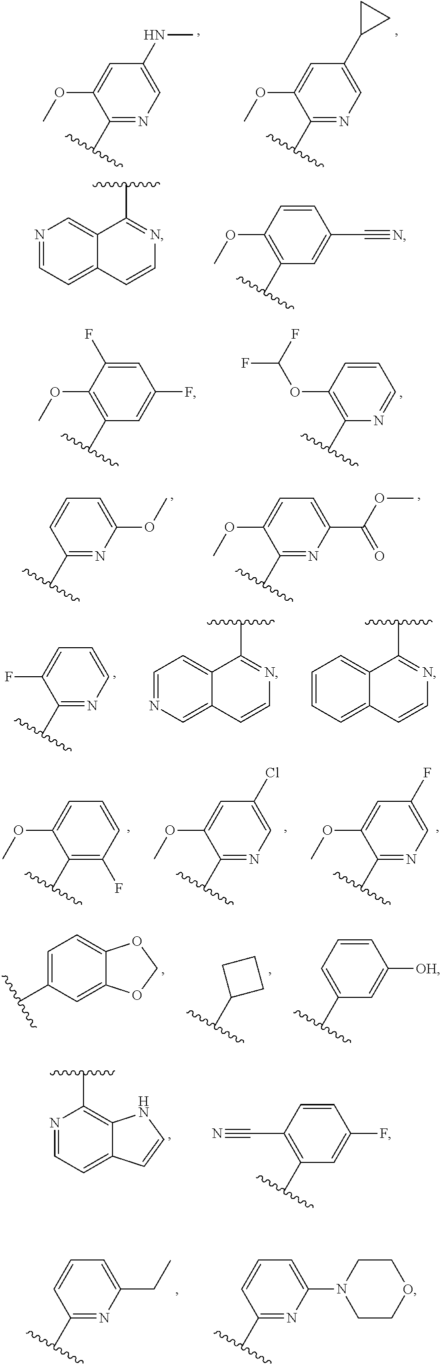

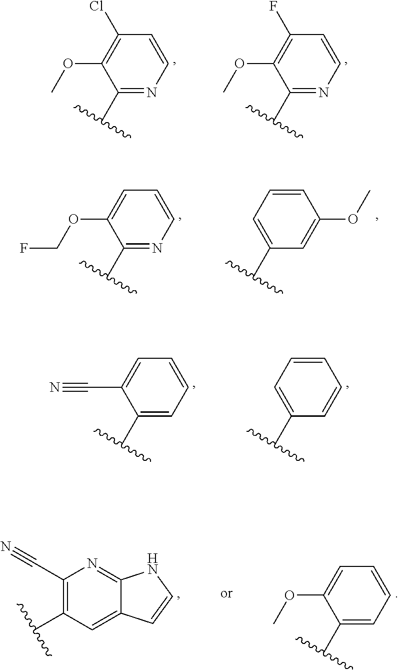

In some embodiments, Cy^{B2} is an optionally substituted 5-15 membered saturated or partially saturated monocyclic, bicyclic, or tricyclic carbocyclyl, heterocyclyl, aryl, or heteroaryl having 1-4 heteroatoms independently selected from nitrogen, oxygen, and sulfur.

In some embodiments, Cy^{B2} is an optionally substituted 10-15 membered saturated or partially saturated tricyclic carbocyclyl, heterocyclyl, aryl, or heteroaryl having 1-4 heteroatoms independently selected from nitrogen, oxygen, and sulfur. In some embodiments, Cy^{B2} is an optionally substituted 10-15 membered saturated or partially saturated tricyclic heterocyclyl having 1-4 heteroatoms independently selected from nitrogen, oxygen, and sulfur. In some embodiments, Cy^{B2} is an optionally substituted 10-15 membered heteroaryl having 1-4 heteroatoms independently selected from nitrogen, oxygen, and sulfur.

In some embodiments, Cy^{B2} is a 13 membered saturated or partially saturated tricyclic carbocyclyl, heterocyclyl, aryl, or heteroaryl having 1-4 heteroatoms independently selected from nitrogen, oxygen, and sulfur. In some embodiments, Cy^{B2} is a 15 membered saturated or partially saturated tricyclic carbocyclyl, heterocyclyl, aryl, or heteroaryl having 1-4 heteroatoms independently selected from nitrogen, oxygen, and sulfur.

As described above and defined herein, each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom or adjacent atoms are optionally taken together with their intervening atoms to form a 3-7 membered saturated or partially unsaturated ring having 0-3 heteroatoms, in addition to the atom or adjacent atoms to which they are attached, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated carbocyclic ring. In some embodiments, R is an optionally substituted 4-7 membered saturated or partially unsaturated heterocyclic ring having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same atom are optionally taken together with their intervening atoms to form a 3-7 membered saturated or partially unsaturated ring having 0-3 heteroatoms, in addition to the atom to which they are attached, independently selected from nitrogen, oxygen, and sulfur. In some embodiments, two R groups on adjacent atoms are optionally taken together with their intervening atoms to form a 3-7 membered saturated or partially unsaturated ring having 0-3 heteroatoms, in addition to the adjacent atoms to which they are attached, independently selected from nitrogen, oxygen, and sulfur.

In some embodiments, R is an optionally substituted 4-7 membered saturated or partially unsaturated carbocyclic ring. In some embodiments, R is an optionally substituted 3 membered saturated or partially unsaturated carbocyclic ring.

In some embodiments, R is as depicted in the compounds of Table 1A, below.

As described above and defined herein, L^{x} is

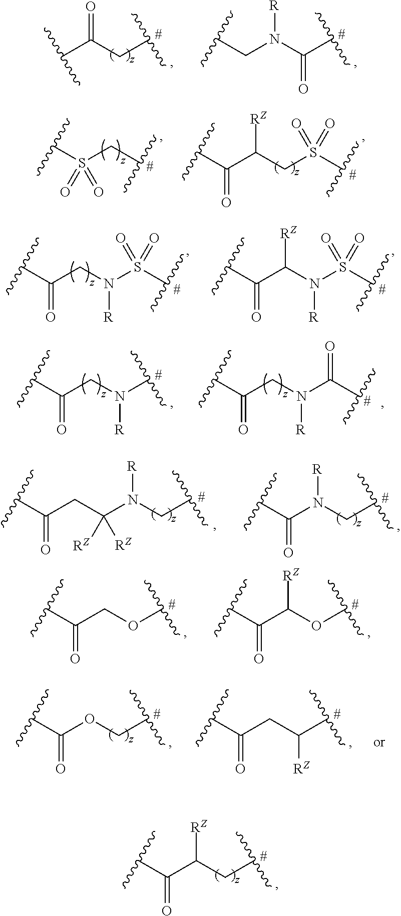

wherein

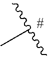

represents a bond to Ring Y.

In some embodiments, L^{x} is

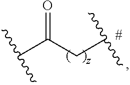

wherein

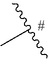

represents a bond to Ring Y.

In some embodiments, L^{x} is —C(O)(CH₂)₁₋₃—.

In some embodiments, L^{x} is

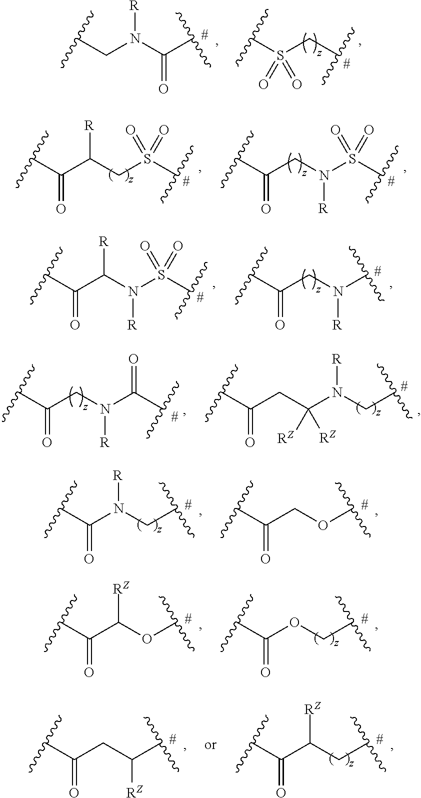

wherein

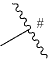

represents a bond to Ring Y.

In some embodiments, L is —C(O)—, —C(O)CH₂—, —S(O)₂CH₂—, —C(O)CH₂CH₂—, —C(O)OCH₂—, —C(O)CH₂O—, —C(O)CH₂CH₂CH₂—, —C(O)CH₂NHC(O)—,

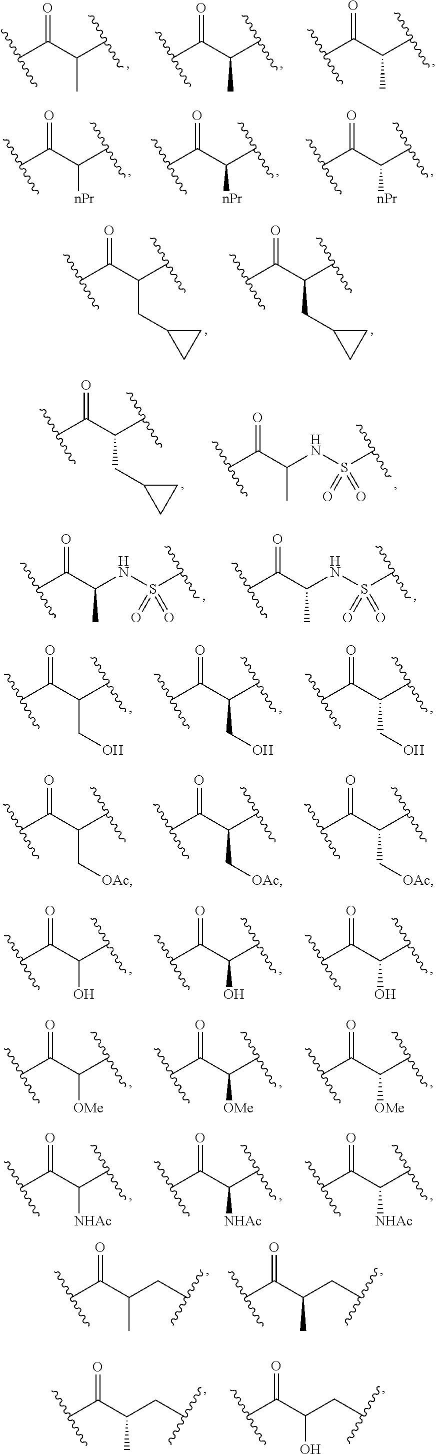

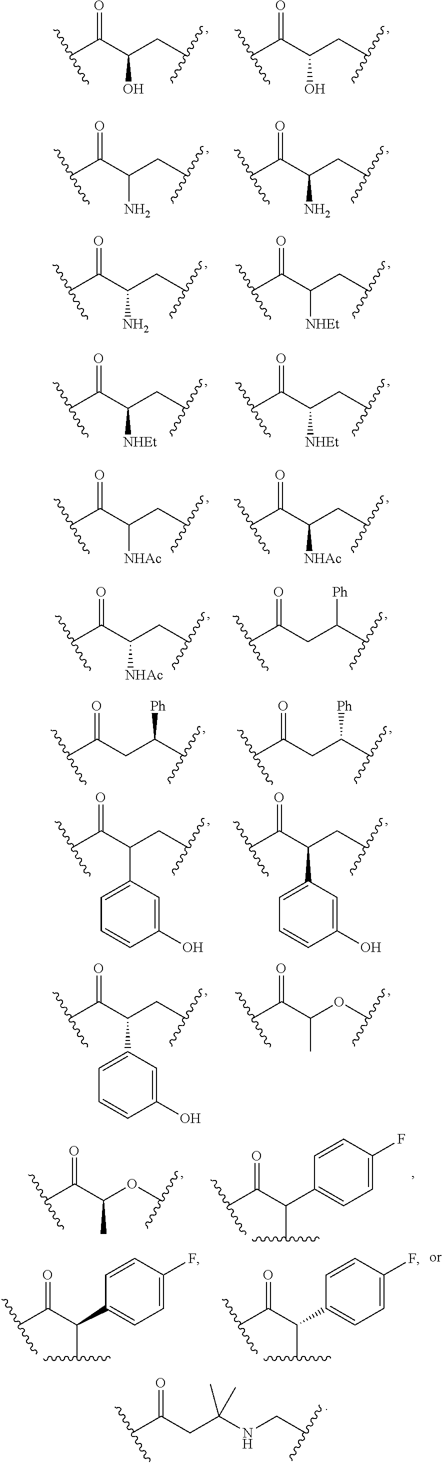

In some embodiments, L^{x} is

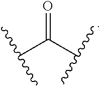 [SMILES: *C(*)=O ; "."]

In some embodiments, L^{x} is —C(O)CH₂CH₂—.

In some embodiments, L^{x} is —S(O)₂CH₂CH₂—.

In some embodiment, L^{x} is as depicted in the compounds of Table 1A, below.

As described above and defined herein, R^{Z} is R^{A}, —OR, or —NR₂. In some embodiments, R^{Z} is R^{A}. In some embodiments, R^{Z} is —OR. In some embodiments, R^{Z} is —NR₂.

As described above and defined herein, each of w, x, y, and z are independently 0, 1, 2, 3, or 4.

In some embodiments, one or more of w, x, and y is 0. In some embodiments, one or more of w, x, and y is 1. In some embodiments, one or more of w, x, and y is 2. In some embodiments, one or more of w, x, and y is 3. In some embodiments, one or more of w, x, and y is 4.

In some embodiments, w is 0. In some embodiments, w is 1. In some embodiments, w is 2. In some embodiments, w is 3. In some embodiments, w is 4.

In some embodiments, x is 0. In some embodiments, x is 1. In some embodiments, x is 2. In some embodiments, x is 3. In some embodiments, x is 4.

In some embodiments, y is 0. In some embodiments, y is 1. In some embodiments, y is 2. In some embodiments, y is 3. In some embodiments, y is 4.

In some embodiments, z is 0. In some embodiments, z is 1. In some embodiments, z is 2. In some embodiments, z is 3. In some embodiments, z is 4. In some embodiments, z is 1, 2, or 3. In some embodiments, z is 2, 3, or 4.

In some embodiment, w, x, y, and z are as depicted in the compounds of Table 1A, below.

In certain embodiments, the present invention provides methods of use related to a compound of formula I, wherein the compound is of formula I-a-1 or I-a-2:

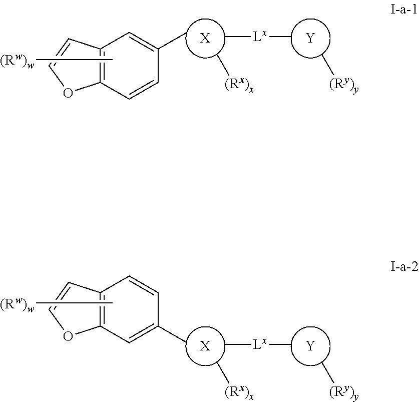

or a pharmaceutically acceptable salt thereof, wherein Ring X, Ring Y, R^{w}, R^{x}, R^{y}, L^{x}, w, x, and y are as defined above and described herein both individually and in combination.

In certain embodiments, the present invention provides methods of use related to a compound of formula I, wherein the compound is of formula I-a-3 or I-a-4:

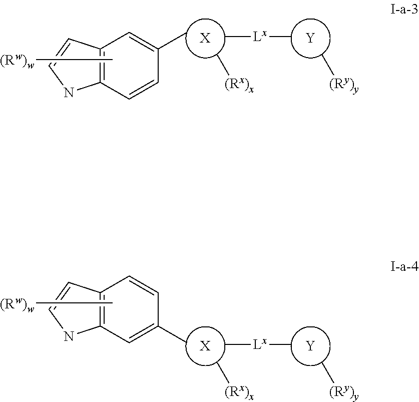

or a pharmaceutically acceptable salt thereof, wherein Ring X, Ring Y, R^{w}, R^{x}, R^{y}, L^{x}, w, x, and y are as defined above and described herein both individually and in combination.

In certain embodiments, the present invention provides methods of use related to a compound of formula I, wherein the compound is of formula I-a-5 or I-a-6:

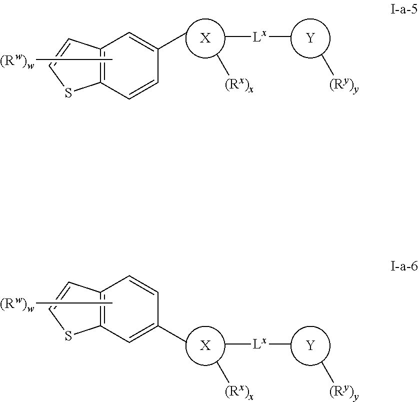

or a pharmaceutically acceptable salt thereof, wherein Ring X, Ring Y, R^{w}, R^{x}, R^{y}, L^{x}, w, x, an y are as defined above and described herein both individually and in combination.

In certain embodiments, the present invention provides methods of use related to a compound of formula I, wherein the compound is of formula I-a-7:

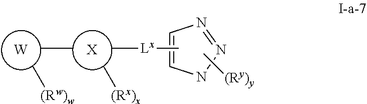

or a pharmaceutically acceptable salt thereof, wherein Ring W, Ring X, R^{w}, R^{x}, R^{y}, L^{x}, w, x, and y are as defined above and described herein both individually and in combination.

In certain embodiments, the present invention provides methods of use related to a compound of formula I, wherein the compound is of formula I-a-8:

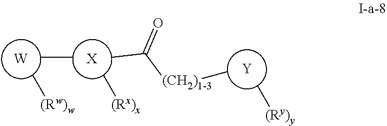

or a pharmaceutically acceptable salt thereof, wherein Ring W, Ring X, Ring Y, R^{w}, R^{x}, R^{y}, w, x, and y are as defined above and described herein both individually and in combination.

In certain embodiments, the present invention provides methods of use related to a compound of formula I, wherein the compound is of formula I-a-9 or I-a-10:

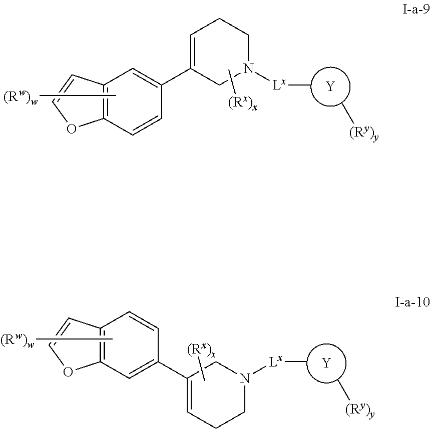

or a pharmaceutically acceptable salt thereof, wherein Ring Y, R^{w}, R^{x}, R^{y}, L^{x}, w, x, and y are as defined above and described herein both individually and in combination.

In certain embodiments, the present invention provides methods of use related to a compound of formula I, wherein the compound is of formula I-a-11 or I-a-12:

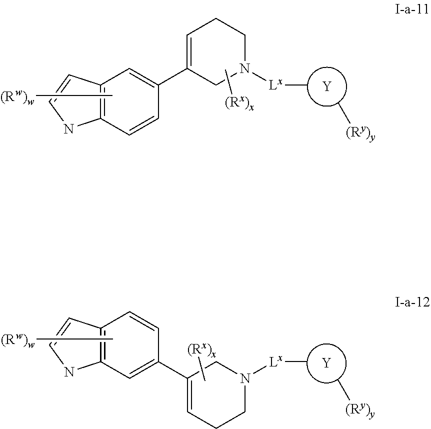

or a pharmaceutically acceptable salt thereof, wherein Ring Y, R^{w}, R^{x}, R^{y}, L^{x}, w, x, and y are as defined above and described herein both individually and in combination.

In certain embodiments, the present invention provides methods of use related to a compound of formula I, wherein the compound is of formula I-a-13 or I-a-14:

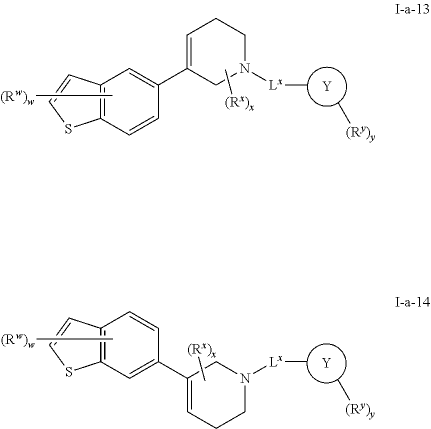

or a pharmaceutically acceptable salt thereof, wherein Ring Y, R^{w}, R^{x}, R^{y}, L^{x}, w, x, and y are as defined above and described herein both individually and in combination.

In certain embodiments, the present invention provides methods of use related to a compound of formula I, wherein the compound is of formula I-a-15:

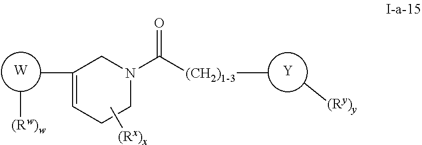

or a pharmaceutically acceptable salt thereof, wherein Ring W, Ring Y, R^{w}, R^{x}, R^{y}, w, x, and y are as defined above and described herein both individually and in combination.

In certain embodiments, the present invention provides methods of use related to a compound of formula I, wherein the compound is of formula I-a-16:

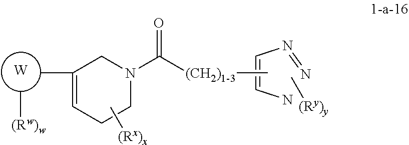

or a pharmaceutically acceptable salt thereof, wherein Ring W, R^{w}, R^{x}, R^{y}, w, x, and y are as defined above and described herein both individually and in combination.

In certain embodiments, the present invention provides methods of use related to a compound of formula I, wherein the compound is of one of formula I-a-1a, I-a-2a, I-a-1b, or I-a-2b:

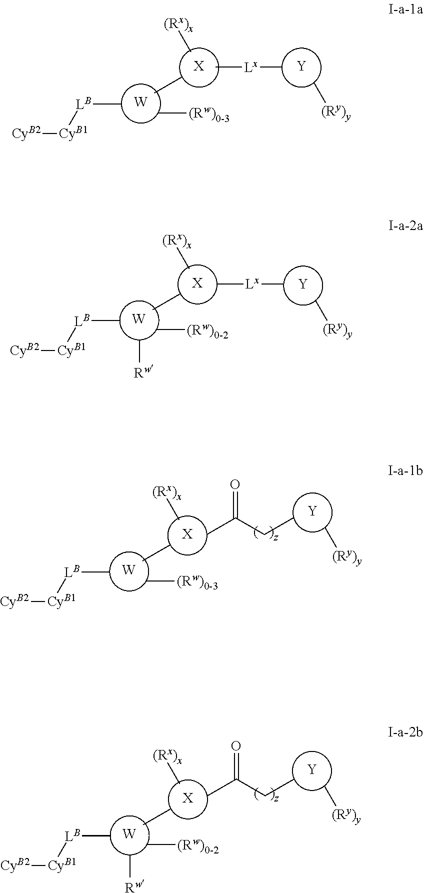

or a pharmaceutically acceptable salt thereof, wherein each variable is as defined above and described herein both individually and in combination, and wherein:
R^{w′} is R^{w}, wherein R^{w} is as defined above and described herein both individually and in combination.

In some embodiments, R^{w′} is an optionally substituted ring selected from phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{w′} is an optionally substituted 3-7 membered saturated or partially unsaturated carbocyclic ring. In some embodiments, R^{w′} is an optionally substituted 3-7 membered saturated or partially unsaturated carbocyclic ring. In some embodiments, R^{w′} is an optionally substituted cyclopropyl. In some embodiments, R^{w′} is an optionally substituted cyclobutyl. In some embodiments, R is an optionally substituted cyclopentyl. In some embodiments, R^{w′} is an optionally substituted cyclohexyl. In some embodiments, R^{w′} is an optionally substituted cyclopropenyl. In some embodiments, R^{w′} is an optionally substituted cyclobutenyl. In some embodiments, R^{w′} is an optionally substituted cyclopentenyl. In some embodiments, R^{w′} is an optionally substituted cyclohexenyl.

In some embodiments, R^{w′} is an optionally substituted 3-7 membered saturated or partially unsaturated heterocyclic ring having 1-2 heteroatoms independently selected from nitrogen, oxygen, and sulfur. In some embodiments, R^{w′} is an optionally substituted 5-6 membered saturated or partially unsaturated heterocyclic ring having 1-2 heteroatoms independently selected from nitrogen, oxygen, and sulfur.

In some embodiments, R^{w′} is an optionally substituted 4-membered saturated or partially unsaturated heterocyclic ring having 1-2 heteroatoms independently selected from nitrogen, oxygen, and sulfur. In some embodiments, R^{w′} is an optionally substituted azetidinyl, oxetanyl, or thietanyl.

In some embodiments, R^{w′} is an optionally substituted 5-membered saturated or partially unsaturated heterocyclic ring having 1-2 heteroatoms independently selected from nitrogen, oxygen, and sulfur. In some embodiments, R^{w′} is an optionally substituted pyrrolidinyl, pyrrolinyl, pyrazolidinyl, pyrazolinyl, imidazolidinyl, or imidazolinyl. In some embodiments, R^{w′} is an optionally substituted dihydropyridinyl, pyrrolidinyl, dihydrofuranyl, tetrahydrofuranyl, dihydrothiophenyl, or tetrahydrothiophenyl.

In some embodiments, R^{w′} is an optionally substituted 6-membered saturated or partially unsaturated heterocyclic ring having 1-2 heteroatoms independently selected from nitrogen, oxygen, and sulfur. In some embodiments, R^{w′} is an optionally substituted piperidinyl, piperazinyl, tetrahydropyranyl, 2H-pyranyl, 4H-pyranyl, 1,4-dioxanyl, 1,4-dioxinyl, thianyl, 2H-thiopyranyl, 4H-thiopyranyl, 1,3-dithanyl, 1,4-dithanyl, morpholinyl, or thiomorpholinyl. In some embodiments, R^{w′} is an optionally substituted dihydropyridinyl, tetrahydropyridinyl, dihydropyranyl, tetrahydropyranyl, dihydrothiopyranyl, or tetrahydrothiopyranyl.

In some embodiments, R^{w′} is an optionally substituted 5-6 membered heteroaryl having 1-4 heteroatoms independently selected from nitrogen, oxygen, and sulfur. In some embodiments, R^{w′} is an optionally substituted 5-membered heteroaryl having 1-4 heteroatoms independently selected from nitrogen, oxygen, and sulfur. In some embodiments, R^{w′} is an optionally substituted pyrazolyl, imidazolyl, triazolyl, or tetrazolyl. In some embodiments, R^{w′} is an optionally substituted imidazolyl, optionally substituted with —C(O)N(R^{◯})₂. In some embodiments, R^{w′} is an optionally substituted furanyl, thiophenyl, oxazolyl, isoxazolyl, isothiazolyl, thiazolyl, oxadiazolyl, or thiadiazolyl. In some embodiments, R^{w′} is furanyl, optionally substituted with —C(O)N(R^{◯})₂.

In some embodiments, R^{w′} is an optionally substituted 6-membered heteroaryl having 1-4 heteroatoms independently selected from nitrogen, oxygen, and sulfur. In some embodiments, R^{w′} is an optionally substituted 6 membered heteroaryl having 1-4 nitrogen heteroatoms. In some embodiments, R^{w′} is optionally substituted pyridinyl, pyrimidinyl, pyridazinyl, or triazinyl. In some embodiments, R^{w′} is an optionally substituted pyridinonyl, pyrazinonyl, or pyrimidinoyl.

In some embodiments, R^{w′} is an optionally substituted 7-11 membered bicyclic heterocyclic ring having 1-4 heteroatoms independently selected from nitrogen, oxygen, and sulfur. In some embodiments, R^{w′} is an optionally substituted 9-10 membered bicyclic heterocyclic ring having 1-4 heteroatoms independently selected from nitrogen, oxygen, and sulfur. In some embodiments, R^{w′} is an optionally substituted benzo[d][1,3]dioxolyl. In some embodiments, R^{w′} is an optionally substituted an 11-membered bicyclic heterocyclic ring having 1-4 heteroatoms independently selected from nitrogen, oxygen, and sulfur.

In some embodiments, R^{w′} is an optionally substituted 8-11 membered bicyclic aryl or heteroaryl having 1-4 heteroatoms independently selected from nitrogen, oxygen and sulfur. In some embodiments, R^{w′} is an optionally substituted naphthalenyl. In some embodiments, R^{w′} is an optionally substituted dihydrobenzodioxepinyl. In some embodiments, R^{w′} is an optionally substituted indenyl or dihydroindenyl.

In some embodiments, R^{w′} is an optionally substituted 9-10 membered bicyclic heteroaryl having 1-4 heteroatoms independently selected from nitrogen, oxygen and sulfur.

In some embodiments, R^{w′} is an optionally substituted 9-membered bicyclic heteroaryl having 1-4 heteroatoms independently selected from nitrogen, oxygen and sulfur. In some embodiments, R^{w′} is an optionally substituted indolyl, benzofuranyl, benzothiophenyl, benzimidazolyl, benzoxazolyl, thienopyridinyl, pyrrolo[3,2-b]pyridinyl, pyrrolo[2,3,-b]pyridinyl, pyrazolyl[1,5-a]pyridinyl, or imidazo[1,2-a]pyridinyl, azaindazolyl (e.g., 4-, 5-, 6-, or 7-azaindazolyl), pyrrolol[2,3-c]pyridinyl or indolizinyl.

In some embodiments, R^{w′} is an optionally substituted 10-membered bicyclic heteroaryl having 1-4 heteroatoms independently selected from nitrogen, oxygen and sulfur. In some embodiments, R^{w′} is an optionally substituted quinolinyl, isoquinolinyl, quinolizinyl, quinoxalinyl, phthalazinyl, quinazolinyl, cinnolinyl, or 1,8-naphthyridinyl.

In some embodiments, R^{w′} is

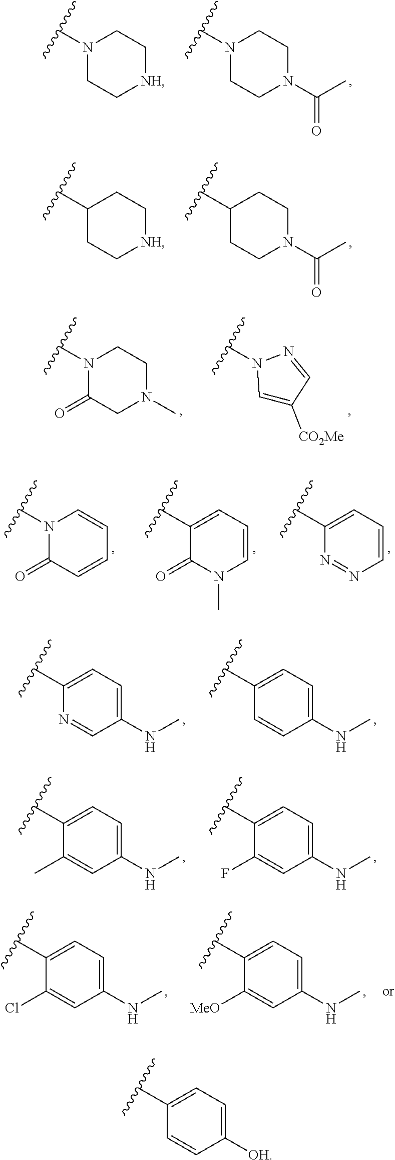

In some embodiments, R^{w′} is

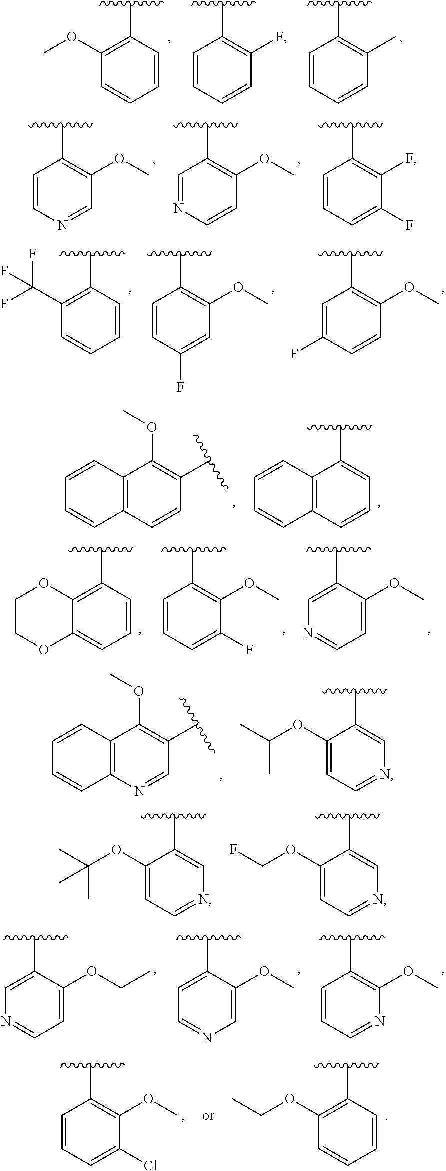

In some embodiments, R^{w′} is

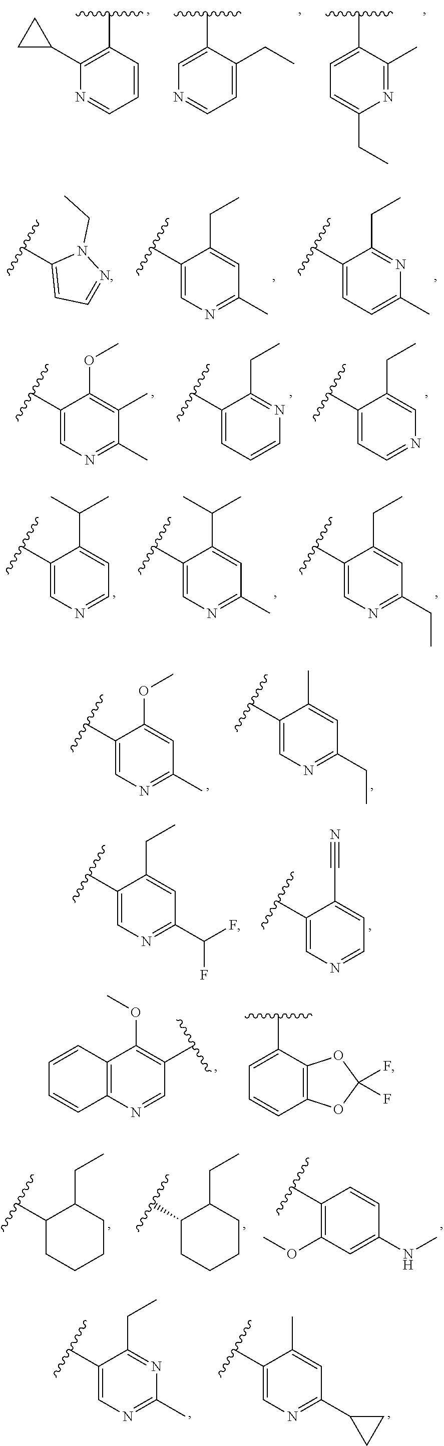

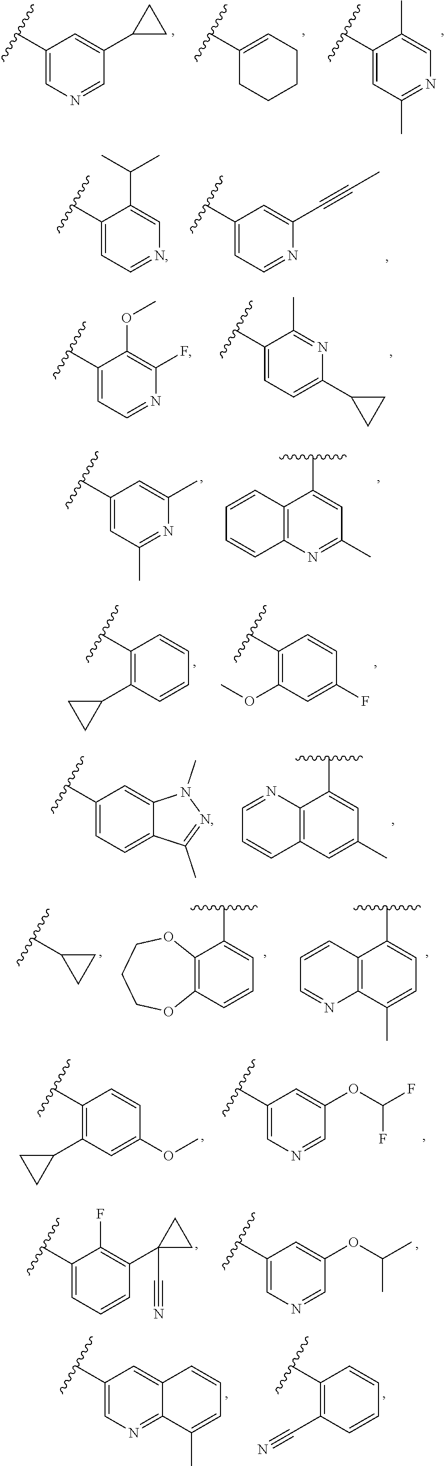

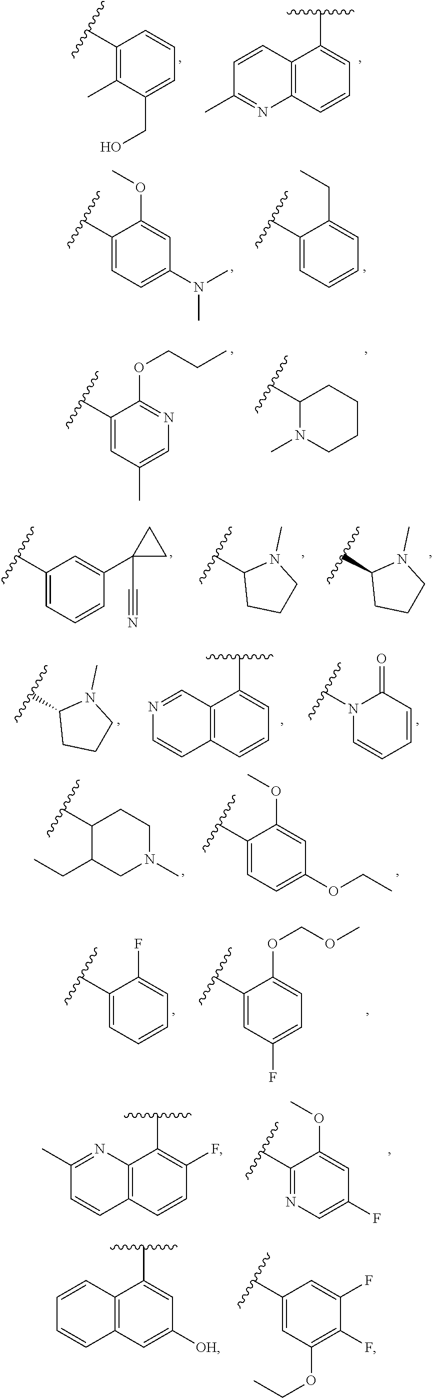

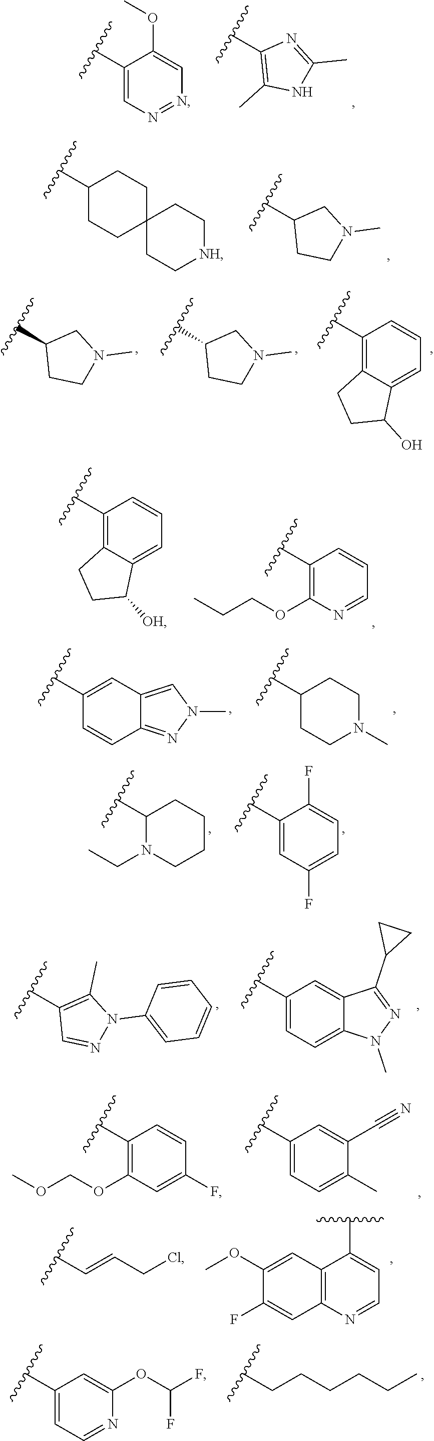

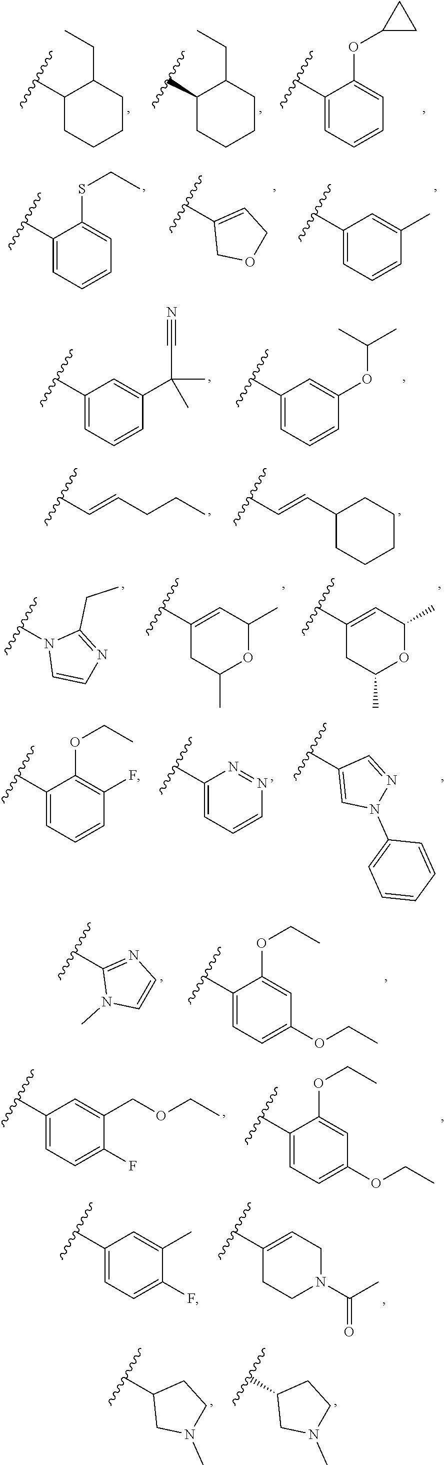

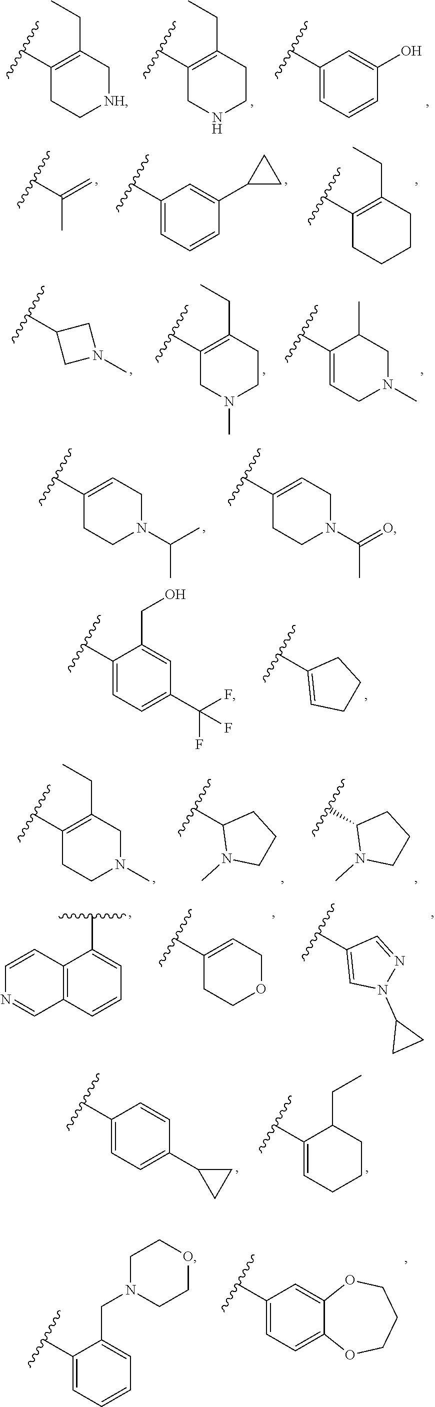

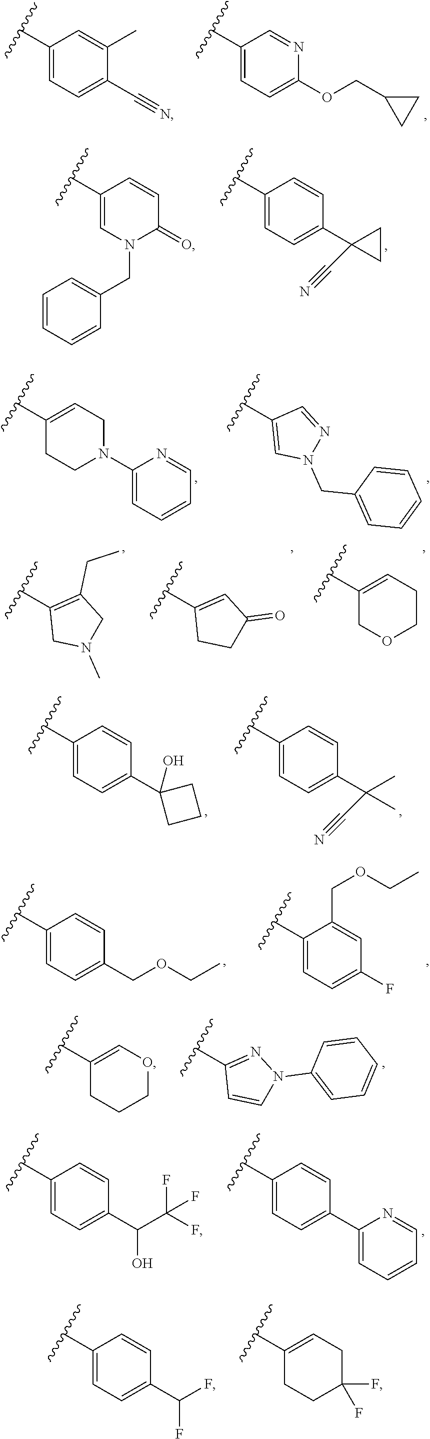

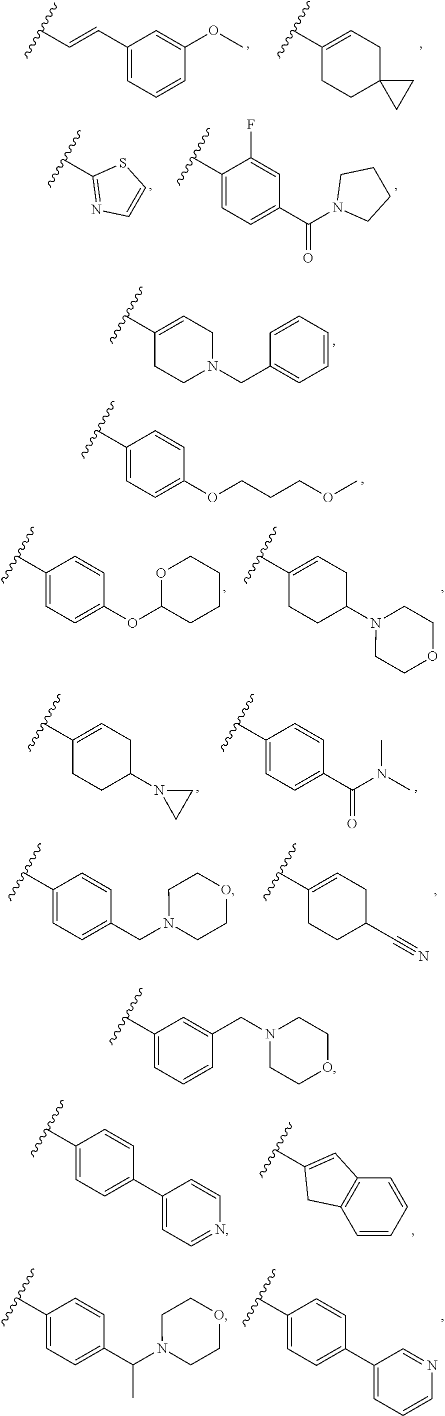

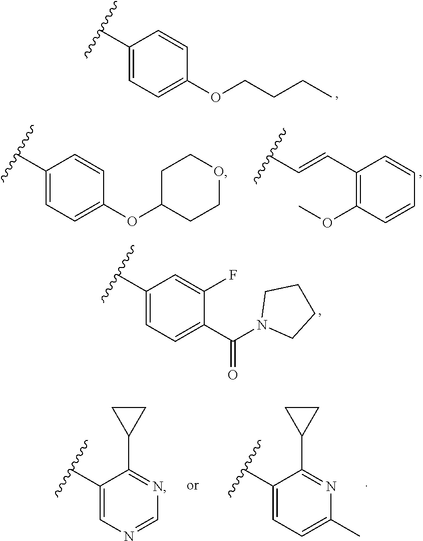

In some embodiments, R^{w′} is an optionally substituted phenyl. In some embodiments, R^{w′} is phenyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is phenyl, optionally substituted with halogen (e.g., fluoro or chloro) or C₁₋₆ aliphatic.

In some embodiments, R^{w′} is an optionally substituted 5 membered saturated or partially unsaturated heterocyclyl having 1-2 heteroatoms independently selected from nitrogen, oxygen, and sulfur. In some embodiments, R^{w′} is an optionally substituted pyrrolidinyl. In some embodiments, R^{w′} is pyrrolidinyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is pyrrolidinyl, optionally substituted with halogen (e.g., fluoro or chloro) or C₁₋₆ aliphatic. In some embodiments, R^{w′} is an optionally substituted 6 membered saturated or partially unsaturated heterocyclyl having 1-2 heteroatoms independently selected from nitrogen, oxygen, and sulfur. In some embodiments, R^{w′} is an optionally substituted piperadinyl. In some embodiments, R^{w′} is piperadinyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is piperadinyl, optionally substituted with halogen (e.g., fluoro or chloro) or C₁₋₆ aliphatic. In some embodiments, R^{w′} is an optionally substituted piperazinyl. In some embodiments, R^{w′} is piperazinyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is piperazinyl, optionally substituted with halogen (e.g., fluoro or chloro) or C₁₋₆ aliphatic.

In some embodiments, R^{w′} is an optionally substituted 5 membered heteroaryl having 1-4 heteroatoms independently selected from nitrogen, oxygen. In some embodiments, R^{w′} is an optionally substituted pyrrolyl, pyrazolyl, imidazolyl, or triazolyl. In some embodiments, R^{w′} is pyrrolyl, pyrazolyl, imidazolyl, or triazolyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is pyrrolyl, pyrazolyl, imidazolyl, or triazolyl, optionally substituted with halogen (e.g., fluoro or chloro) or C₁₋₆ aliphatic. In some embodiments, R^{w′} is an optionally substituted pyrazolyl. In some embodiments, R^{w′} is pyrazolyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is pyrazolyl, optionally substituted with halogen (e.g., fluoro or chloro) or C₁₋₆ aliphatic. In some embodiments, R^{w′} is an optionally substituted imidazolyl. In some embodiments, R^{w′} is imidazolyl, optionally substituted with halogen (e.g., fluoro or chloro), —CN, C₁₋₆ aliphatic optionally further substituted with halogen (e.g., fluoro or chloro), or —OR^{◯}, wherein R^{◯} is hydrogen or C₁₋₆ aliphatic. In some embodiments, R^{w′} is imidazolyl, optionally substituted with halogen (e.g., fluoro or chloro) or C₁₋₆ aliphatic.

In some embodiments, R^{w′} is an optionally substituted 6 membered heteroaryl having 1-4 heteroatoms independently selected from nitrogen, oxygen. In some embodiments, R^{w′} is an optionally substituted pyridinyl, pyridazinyl, pyrimidinyl, pyrazinyl, or triazinyl. In some embodiments, R^{w′} is pyridinyl, pyridazinyl, pyrimidinyl, pyrazinyl, or triazinyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is pyridinyl, pyridazinyl, pyrimidinyl, pyrazinyl, or triazinyl, optionally substituted with halogen (e.g., fluoro or chloro) or C₁₋₆ aliphatic. In some embodiments, R^{w′} is an optionally substituted pyridinyl. In some embodiments, R^{w′} is pyridinyl, optionally substituted with halogen (e.g., fluoro or chloro), —CN, C₁₋₆ aliphatic optionally further substituted with halogen (e.g., fluoro or chloro), —S(O)₂R^{◯}, or —OR⁰, wherein R^{◯} is hydrogen or C₁₋₆ aliphatic. In some embodiments, R^{w′} is pyridinyl, optionally substituted with halogen (e.g., fluoro or chloro) or C₁₋₆ aliphatic. In some embodiments, R^{w′} is an optionally substituted pyridazinyl. In some embodiments, R is pyridazinyl, optionally substituted with halogen (e.g., fluoro or chloro), —CN, C₁₋₆ aliphatic optionally further substituted with halogen (e.g., fluoro or chloro), —S(O)₂R^{◯}, or —OR⁰, wherein R^{◯} is hydrogen or C₁₋₆ aliphatic. In some embodiments, R^{w′} is pyridazinyl, optionally substituted with halogen (e.g., fluoro or chloro) or C₁₋₆ aliphatic. In some embodiments, R^{w′} is an optionally substituted pyridinonyl, pyridazinonyl, pyrimidinonyl, pyrazinonyl, or triazinonyl. In some embodiments, R^{w′} is pyridinonyl, pyridazinonyl, pyrimidinonyl, pyrazinonyl, or triazinonyl, optionally substituted with halogen (e.g., fluoro or chloro), —CN, C₁₋₆ aliphatic optionally further substituted with halogen (e.g., fluoro or chloro), —S(O)₂R^{◯}, or —OR⁰, wherein R^{◯} is hydrogen or C₁₋₆ aliphatic. In some embodiments, R^{w′} is pyridinonyl, pyridazinonyl, pyrimidinonyl, pyrazinonyl, or triazinonyl, optionally substituted with halogen (e.g., fluoro or chloro) or C₁₋₆ aliphatic. In some embodiments, R^{w′} is an optionally substituted pyridinonyl. In some embodiments, R^{w′} is pyridinonyl, optionally substituted with halogen (e.g., fluoro or chloro), —CN, C₁₋₆ aliphatic optionally further substituted with halogen (e.g., fluoro or chloro), —S(O)₂R^{◯}, or —OR⁰, wherein R^{◯} is hydrogen or C₁₋₆ aliphatic. In some embodiments, R^{w′} is pyridinonyl, optionally substituted with halogen (e.g., fluoro or chloro) or C₁₋₆ aliphatic.

In some embodiments, R^{w′} is an optionally substituted cyclopropyl. In some embodiments, R^{w′} is an optionally substituted 5 membered saturated or partially unsaturated carbocyclyl. In some embodiments, R^{w′} is an optionally substituted cyclopentanyl or cyclopentenyl. In some embodiments, R is an optionally substituted 6 membered saturated or partially unsaturated carbocyclyl. In some embodiments, R^{w′} is an optionally substituted cyclohexanyl or cyclohexenyl. In some embodiments, R^{w′} is an optionally substituted naphthalenyl.

In some embodiments, R^{w′} is an optionally substituted 9 membered bicyclic heteroaryl having 1-5 heteroatoms independently selected from nitrogen, oxygen, and sulfur. In some embodiments, R^{w′} is an optionally substituted indolyl, azaindolyl, isoindolyl, azaisoindolyl, indazolyl, azaindazolyl, benzimidazolyl, or azabenzimidazolyl. In some embodiments, R^{w′} is an optionally substituted indolyl. In some embodiments, R^{w′} is an optionally substituted benzothiophenyl, benzofuranyl, isobenzofuranyl, benzoisooxazolyl, benzoisothiazolyl, benzoxazolyl, benzothiazolyl, or benzothiadiazolyl.

In some embodiments, R^{w′} is an optionally substituted 8-11 membered saturated or partially unsaturated spiro heterocyclyl having 1-3 heteroatoms independently selected from nitrogen, oxygen, and sulfur. In some embodiments, R^{w′} is an optionally substituted 9-11 membered saturated or partially unsaturated spiro heterocyclyl having 1-3 heteroatoms independently selected from nitrogen, oxygen, and sulfur. In some embodiments, R^{w′} is an optionally substituted 9 membered saturated or partially unsaturated spiro heterocyclyl having 1-3 heteroatoms independently selected from nitrogen, oxygen, and sulfur. In some embodiments, R^{w′} is an optionally substituted 9 membered saturated or partially unsaturated spiro heterocyclyl having 1-3 nitrogen heteroatoms. In some embodiments, R^{w′} is an optionally substituted 4,6-spiro heterocyclyl having 1-3 heteroatoms independently selected from nitrogen, oxygen, and sulfur.

In some embodiments, R^{w′} is an optionally substituted 10 membered saturated or partially unsaturated spiro heterocyclyl having 1-3 heteroatoms independently selected from nitrogen, oxygen, and sulfur. In some embodiments, R^{w′} is an optionally substituted 10 membered saturated or partially unsaturated spiro heterocyclyl having 1-3 nitrogen heteroatoms. In some embodiments, R^{w′} is an optionally substituted 5,6-spiro heterocyclyl having 1-3 heteroatoms independently selected from nitrogen, oxygen, and sulfur.

In some embodiments, R^{w′} is an optionally substituted 11 membered saturated or partially unsaturated spiro heterocyclyl having 1-3 heteroatoms independently selected from nitrogen, oxygen, and sulfur. In some embodiments, R^{w′} is an optionally substituted 11 membered saturated or partially unsaturated spiro heterocyclyl having 1-3 nitrogen heteroatoms. In some embodiments, R^{w′} is an optionally substituted 6,6-spiro heterocyclyl having 1-3 heteroatoms independently selected from nitrogen, oxygen, and sulfur.

In certain embodiments, the present invention provides methods of use related to a compound of formula I, wherein the compound is of one of formula I-b-1 through I-b-6:

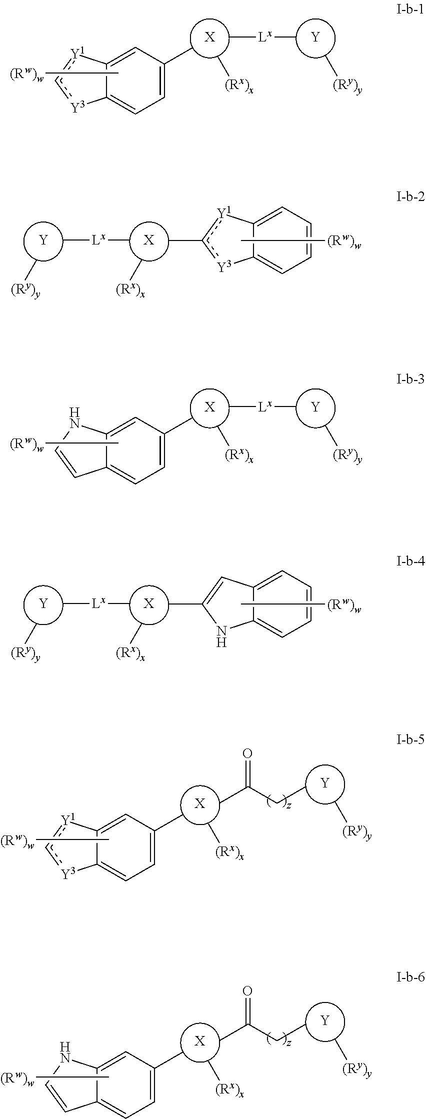

or a pharmaceutically acceptable salt thereof, wherein each variable is as defined above and described herein both individually and in combination.

In certain embodiments, the present invention provides methods of use related to a compound of formula I, wherein the compound is of one of formula I-c-1 through I-c-6:

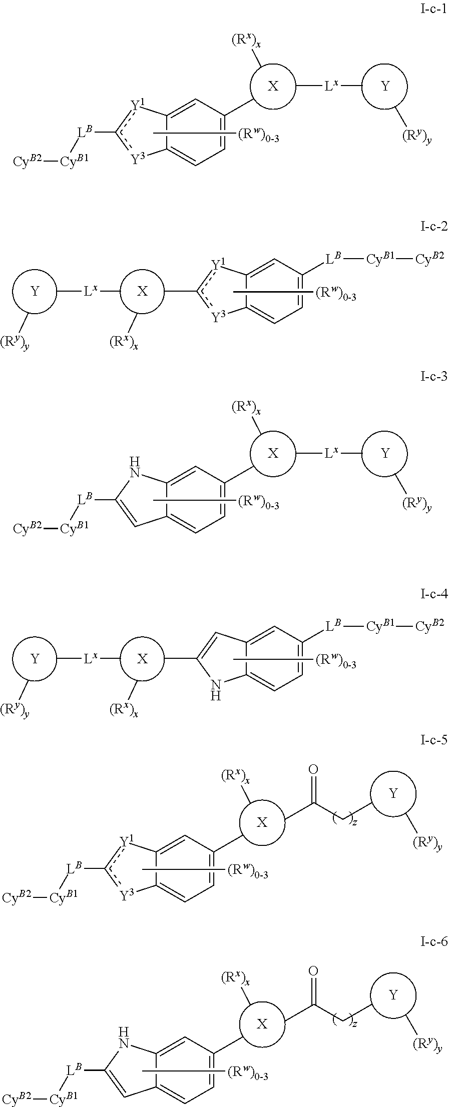

or a pharmaceutically acceptable salt thereof, wherein each variable is as defined above and described herein both individually and in combination.

In certain embodiments, the present invention provides methods of use related to a compound of formula I, wherein the compound is of one of formula I-d-1 through I-d-6:

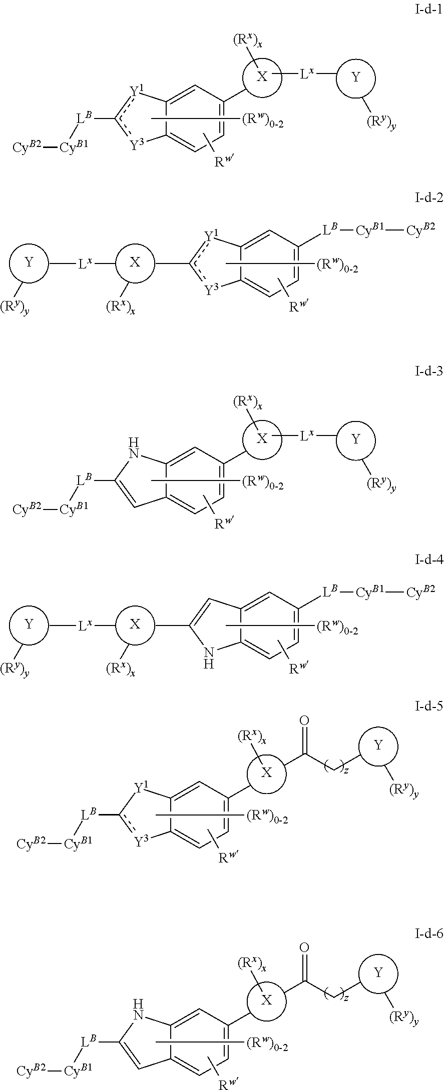

or a pharmaceutically acceptable salt thereof, wherein each variable is as defined above and described herein both individually and in combination.

In certain embodiments, the present invention provides methods of use related to a compound of formula I, wherein the compound is of one of formula I-e-1 through I-e-6:

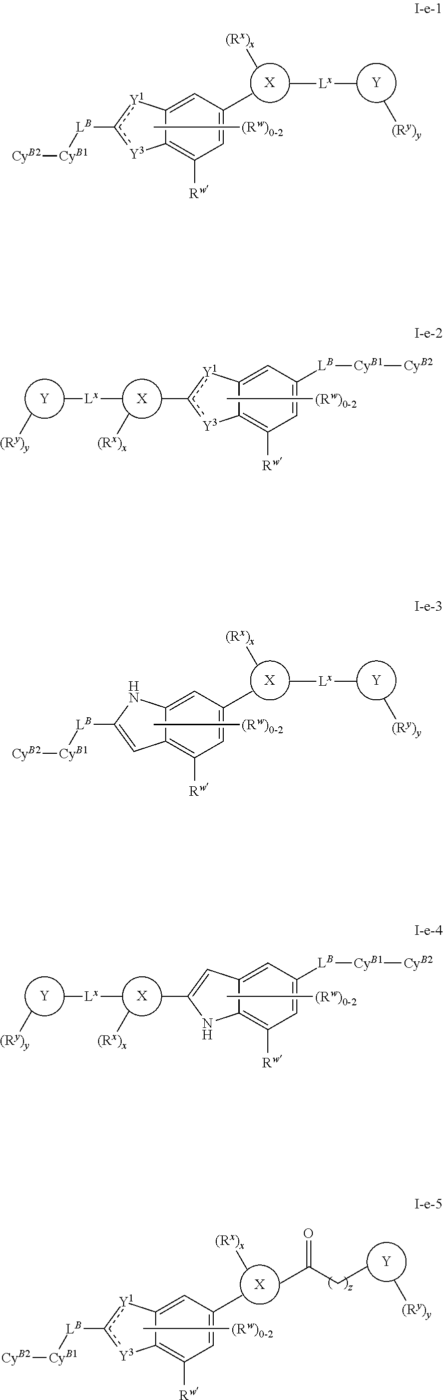

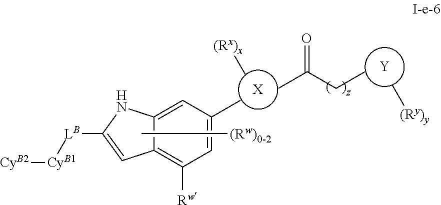

or a pharmaceutically acceptable salt thereof, wherein each variable is as defined above and described herein both individually and in combination.

Exemplary compounds for use in provided methods of the invention are set forth in Table 1A and Table 2A, below.

Lengthy table referenced here
US20250214977A1-20250703-T00001
Please refer to the end of the specification for access instructions.

Lengthy table referenced here
US20250214977A1-20250703-T00002
Please refer to the end of the specification for access instructions.

In some embodiments, the present invention provides methods of inhibiting STAT6, or a mutant thereof, comprising administering a compound set forth in Table 1A, above, or a pharmaceutically acceptable salt thereof. In some embodiments, the present invention provides methods of inhibiting STAT6, or a mutant thereof, comprising administering a compound set forth in Table 1A, above.

In some embodiments, the present invention provides methods of treating a STAT6-mediated disease, disorder, or condition described herein, comprising administering a compound set forth in Table 1A, above, or a pharmaceutically acceptable salt thereof. In some embodiments, the present invention provides methods of treating a STAT6-mediated disease, disorder, or condition described herein, comprising administering a compound set forth in Table 1A, above.

In some embodiments, the present invention provides methods of inhibiting STAT6, or a mutant thereof, comprising administering a compound set forth in Table 2A, above, or a pharmaceutically acceptable salt thereof. In some embodiments, the present invention provides methods of inhibiting STAT6, or a mutant thereof, comprising administering a compound set forth in Table 2A, above.

In some embodiments, the present invention provides methods of treating a STAT6-mediated disease, disorder, or condition described herein, comprising administering a compound set forth in Table 2A, above, or a pharmaceutically acceptable salt thereof. In some embodiments, the present invention provides methods of treating a STAT6-mediated disease, disorder, or condition described herein, comprising administering a compound set forth in Table 2A, above.

In some embodiments, the present invention provides methods of inhibiting STAT6, or a mutant thereof, comprising administering a compound described herein (e.g., a compound of formula I, I′, II, III, or IV), or a pharmaceutically acceptable salt thereof. In some embodiments, the present invention provides methods of inhibiting STAT6, or a mutant thereof, comprising administering a compound described herein (e.g., a compound of formula I, I′, II, III, or IV).

In some embodiments, the present invention provides methods of treating a STAT6-mediated disease, disorder, or condition described herein, comprising administering a compound described herein (e.g., a compound of formula I, I′, II, III, or IV), or a pharmaceutically acceptable salt thereof. In some embodiments, the present invention provides methods of treating a STAT6-mediated disease, disorder, or condition described herein, comprising administering a compound described herein (e.g., a compound of formula I, I′, II, III, or IV).

In some embodiments, the present invention provides a compound described herein (such as a compound of formula I, I′, II, III, or IV), or a pharmaceutically acceptable salt thereof, or a pharmaceutical composition comprising a compound described herein (such as a compound of formula I, I′, II, III, or IV), or a pharmaceutically acceptable salt thereof, and a pharmaceutically acceptable carrier, adjuvant, or vehicle for use as a medicament for inhibiting STAT6, or a mutant thereof. In some embodiments, the present invention provides a compound described herein (such as a compound of formula I, I′, II, III, or IV), or a pharmaceutically acceptable salt thereof, or a pharmaceutical composition comprising a compound described herein (such as a compound of formula I, I′, II, III, or IV), or a pharmaceutically acceptable salt thereof, and a pharmaceutically acceptable carrier, adjuvant, or vehicle for use as a medicament for treating a STAT6-mediated disease, disorder, or condition described herein.

In some embodiments, the invention also provides a compound described herein (such as a compound of formula I, I′, II, III, or IV), or pharmaceutical compositions described herein, for use in a method for modulating (e.g., inhibiting) STAT6 as described herein. In some embodiments, the invention also provides a compound described herein (such as a compound of formula I, I′, II, III, or IV), or pharmaceutical compositions described herein, for use in a method for treating a STAT6-mediated disorder as described herein.

### Uses of Compounds and Pharmaceutically Acceptable Compositions

Compounds and compositions described herein are generally useful for the modulation of STAT6 protein activity including phosphorylated or activated STAT6 protein (e.g., pSTAT6) activity.

According to one embodiment, the invention relates to a method of modulating (e.g., inhibiting) STAT6 or a mutant thereof, activity in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. In some embodiments, the STAT6 is from a biological sample. In some embodiments, the biological sample is taken from a subject.

Inhibition of STAT6, or a mutant thereof, activity in a biological sample is useful for a variety of purposes that are known to one of skill in the art. Examples of such purposes include, but are not limited to, blood transfusion, organ-transplantation, biological specimen storage, and biological assays.

According to another embodiment, the invention relates to a method of modulating (e.g., inhibiting) STAT6, or a mutant thereof, activity in a patient comprising the step of administering to said patient a compound of the present invention, or a composition comprising said compound. In other embodiments, the present invention provides a method for treating a disorder mediated by STAT6 or a mutant thereof, in a patient in need thereof, comprising the step of administering to said patient a compound according to the present invention or pharmaceutically acceptable composition thereof. Such disorders are described in detail herein.

The activity of a compound utilized in this invention as a modulator (e.g., inhibitor) of STAT6 or a mutant thereof, may be assayed in vitro, in vivo or in a cell line. In vitro assays include assays that determine inhibition of either the activity and/or the subsequent functional consequences of activated STAT6 protein or a mutant thereof. Alternate in vitro assays quantitate the ability of the inhibitor to bind to STAT6 protein. Inhibitor binding may be measured by radiolabeling the inhibitor prior to binding, isolating the inhibitor/STAT6 complex and determining the amount of radiolabel bound. Alternatively, inhibitor binding may be determined by running a competition experiment where new inhibitors are incubated with STAT6 protein bound to known radioligands. Detailed conditions for assaying a compound utilized in this invention as a modulator (e.g., inhibitor) of STAT proteins, or a mutant thereof, are set forth in the Examples below.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are modulators (e.g., inhibitors) of STAT6 protein and are therefore useful for treating one or more disorders associated with activity of STAT6 protein. Thus, in certain embodiments, the present invention provides a method for treating a STAT6-mediated disorder comprising the step of administering to a patient in need thereof a compound of the present invention, or pharmaceutically acceptable composition thereof.

As used herein, the term “STAT6-mediated” disorders, diseases, and/or conditions as used herein means any disease or other deleterious condition in which STAT6 or a mutant thereof, are known to play a role. Accordingly, another embodiment of the present invention relates to treating or lessening the severity of one or more diseases in which STAT6 or a mutant thereof, are known to play a role.

STAT6 functions as a transcription factor to induce gene expression and plays an important role in the IL-4/IL-13 signaling pathway and thus is critical in IL-4/IL-13 mediated biological responses including in human malignancies (e.g., Patel, B. K. R., et al. “Localization of the human stat6 gene to chromosome 12q13. 3-q14. 1, a region implicated in multiple solid tumors.” Genomics 52.2 (1998): 192-200). The STAT6-mediated signaling pathway has been shown to be required for the development of T-helper type 2 (Tb2) cells and Th2 immune response and plays a critical role in Th2 lung inflammatory responses including clearance of parasitic infections and in the pathogenesis of asthma (e.g., Walford, H. H. and Doherty. T. A. “STAT6 and lung inflammation.” Jak-stat 2.4 (2013): e25301). It has been found that STAT6 induces the expression of BCL2L1/BCL-X(L), which is responsible for the anti-apoptotic activity of IL-4 and is shown to play a prominent role in adaptive immunity such as providing innate immune signaling in response to virus infection (e.g., Chen, H., et al. “Activation of STAT6 by STING is critical for antiviral innate immunity.” Cell 147.2 (2011): 436-446). Knockout studies in mice have suggested the role STAT6 in differentiation of T helper 2 (Th2), expression of cell surface markers, and class switch of immunoglobulins. STAT6 protein also regulates other transcription factor as Gata3, which is important regulator of Th2 differentiation. STAT6 is also required for the development of IL-9-secreting T cells.

In some embodiments, biomarkers associated with the IL-4/13 pathway include IgE, Thymus and activation regulated chemokine (TARC), CD23, periostin, and eisinophils. TARC is a serum TH2 biomarker and chemoattractant for TH2 cell. CD23 is a B cell activation marker and correlates with IgE class switch. Periostin is a serum TH2 biomarker and ECM protein associated with tissue remodeling in atopic diseases.

In some embodiments, treatment with a provided compound results in lesser IL-4 induced TARC release compared to a reference or standard level. In some embodiments, treatment with a provided compound results in lesser IL-13 induced CD23 expression compared to a reference or standard level. In some embodiments, treatment with a provided compound results in lesser IL-13 induced periostin release compared to a reference or standard level.

In some embodiments, treatment with a provided compound inhibits IL-4 induced TARC release. In some embodiments, treatment with a provided compound inhibits IL-13 induced CD23 expression. ISE, treatment with a provided compound inhibits IL-13 induced periostin release.

In some embodiments, the present invention provides a method for treating one or more disorders, diseases, and/or conditions wherein the disorder, disease, or condition is a neurodegenerative disorder, a viral disease, an autoimmune disease, an inflammatory disorder, conditions associated with organ transplantation, immunodeficiency disorders, an infectious disease, thrombin-induced platelet aggregation, liver disease, or pathologic immune conditions involving T cell activation.

Diseases and conditions treatable according to the methods of this invention include, but are not limited to, viral disease, autoimmune diseases, autoinflammatory syndromes, atherosclerosis, psoriasis, allergic disorders, inflammatory bowel disease, inflammation, acute and chronic gout and gouty arthritis, neurological disorders, immunodeficiency disorders such as AIDS and HIV, osteoarthritis, infectious diseases, and pathologic immune conditions involving T cell activation in a patient. In one embodiment, a human patient is treated with a compound of the current invention and a pharmaceutically acceptable carrier, adjuvant, or vehicle, wherein said compound is present in an amount to measurably modulate (e.g., inhibit) STAT6 or a mutant thereof

Compounds according to the invention are useful in the treatment of inflammatory or obstructive airways diseases, resulting, for example, in reduction of tissue damage, airways inflammation, bronchial hyperreactivity, remodeling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma exacerbated or induced following bacterial or viral infection. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics.

Another aspect of the present invention relates to a method of treating an allergic or inflammatory disease in a subject comprising administering to the subject a therapeutically effective amount of a compound of the present invention to the subject. The disease may be a lung disease such as, e.g., asthma, airway hyperresponsiveness (AHR), an allergic disease, allergic rhinitis, emphysema, chronic obstructive pulmonary disease (COPD), reactive airway disease, chronic rhinosinusitis, or essentially any other disease of the upper or lower airways that produces airflow obstruction.

Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, such as therapy for or intended to restrict or abort symptomatic attack when it occurs, for example antiinflammatory or bronchodilatory. Prophylactic benefit in asthma may in particular be apparent in subjects prone to “morning dipping”. “Morning dipping” is a recognized asthmatic syndrome, common to a substantial percentage of asthmatics and characterized by asthma attack, e.g., between the hours of about 4 to 6 am, i.e., at a time normally substantially distant form any previously administered symptomatic asthma therapy.

In some embodiments, STAT6, via its Src homology 2 (SH2) domain, is recruited to the phosphotyrosine residues and is phosphorylated on Tyr641. In some embodiments, STAT6 then dimerizes via reciprocal SH2 domain-pTyr641 interactions, translocates to the nucleus, and participates in the expression of genes leading to asthma and airway hyperresponsiveness (AHR).

In some embodiments, the present invention provides a method of treating asthma in a patient in need thereof, comprising administering a compound of the present invention, or a pharmaceutically acceptable salt thereof.

In some embodiments, the present invention provides a method of treating airway hyperresponsiveness (AHR) in a patient in need thereof, comprising administering a compound of the present invention, or a pharmaceutically acceptable salt thereof.

In some embodiments, the present invention provides a method of treating allergic rhinitis in a patient in need thereof, comprising administering a compound of the present invention, or a pharmaceutically acceptable salt thereof.

In some embodiments, the present invention provides a method of treating allergic asthma in a patient in need thereof, comprising administering a compound of the present invention, or a pharmaceutically acceptable salt thereof.

In some embodiments, the present invention provides a method of treating emphysema in a patient in need thereof, comprising administering a compound of the present invention, or a pharmaceutically acceptable salt thereof.

In some embodiments, the present invention provides a method of treating chronic rhinosinusitis in a patient in need thereof, comprising administering a compound of the present invention, or a pharmaceutically acceptable salt thereof.

In some embodiments, the present invention provides a method of treating COPD in a patient in need thereof, comprising administering a compound of the present invention, or a pharmaceutically acceptable salt thereof.

Compounds of the current invention can be used for other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, but not limited to, acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

With regard to their anti-inflammatory activity, in particular in relation to inhibition of eosinophil activation, compounds of the invention are also useful in the treatment of eosinophil related disorders, e.g., eosinophilia, in particular eosinophil related disorders of the airways (e.g., involving morbid eosinophilic infiltration of pulmonary tissues) including hypereosinophilia as it effects the airways and/or lungs as well as, for example, eosinophil-related disorders of the airways consequential or concomitant to Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Compounds of the invention are also useful in the treatment of inflammatory or allergic conditions of the skin. In some embodiments, the present invention provides a method of treating inflammatory or allergic conditions of the skin in a patient in need thereof, comprising administering a compound of the present invention, or a pharmaceutically acceptable salt thereof.

In some embodiments the inflammatory disease of the skin is selected from psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds of the invention may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g., ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis or primary biliary cholangitis, uveitis (anterior and posterior), Sjogren's syndrome, keratoconjunctivitis sicca and vernal keratoconjunctivitis, interstitial lung disease or fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, encephalomyelitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments, the present invention provides a method of treating an autoimmune disease selected from encephalomyelitis, systemic sclerosis, idiopathic pulmonary fibrosis (IPF), inflammatory bowel disease, atopic dermatitis, rheumatoid arthritis, graft versus host disease (acute and chronic), and other tissue fibrosis diseases.

In some embodiments, the present invention provides a method of treating idiopathic interstitial pneumonia(s) (IIPs), including any type of lung fibrosis, either interstitial lung disease associated with rheumatic disease (including SSc) or IPF itself, in a patient in need thereof, comprising administering a compound of the present invention, or a pharmaceutically acceptable salt thereof.

In some embodiments the inflammatory disease which can be treated according to the methods of this invention is selected from acute and chronic gout, chronic gouty arthritis, psoriasis, psoriatic arthritis, rheumatoid arthritis, Juvenile rheumatoid arthritis, systemic juvenile idiopathic arthritis (SJIA), cryopyrin associated periodic syndrome (CAPS), and osteoarthritis.

In some embodiments the inflammatory disease which can be treated according to the methods of this invention is a TH17 mediated disease or TH17-associated disease. In some embodiments the TH17 mediated disease or TH17-associated disease is selected from psoriasis, psoriatric arthritis, systemic lupus erythematosus, multiple sclerosis, and inflammatory bowel disease (including Crohn's disease or ulcerative colitis), or graft-versus-host disease.

In some embodiments the inflammatory disease which can be treated according to the methods of this invention is selected from Sjogren's syndrome, allergic disorders, osteoarthritis, conditions of the eye such as ocular allergy, conjunctivitis, keratoconjunctivitis sicca and vernal conjunctivitis, and diseases affecting the nose such as allergic rhinitis.

In some embodiments, the present invention provides a method of treating an autoimmune disease or inflammatory disorder is selected from nonalcoholic fatty liver disease (NAFLD), nonalcoholic steatohepatitis (NASH), idiopathic autoimmune hepatitis, progressive fibrosis associated interstitial lung disease, pulmonary arterial hypertension (PAH), immunoglobulin G4-related disease (IgG4-RD), chronic organ rejection (e.g., lung transplant), vasculitides (e.g., vasculitides), and STAT6 gain of function (GOF) mutations.

In some embodiments, the present invention provides a method of treating STAT6 gain of function (GOF) mutations in a patient in need thereof, comprising administering a compound of the present invention, or a pharmaceutically acceptable salt thereof. In some embodiments, the STAT6 GOF mutation is STAT6VT.

In some embodiments, the cardiovascular disease which can be treated according to the methods of the present invention include, but are not limited to, restenosis, cardiomegaly, atherosclerosis, myocardial infarction, ischemic stroke, congestive heart failure, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, and deep venous thrombosis.

In some embodiments, the neurodegenerative disease which can be treated according to the methods of the present invention include, but are not limited to, Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

In some embodiments the invention provides a method of treating, preventing or lessening the severity of Alzheimer's disease comprising administering to a patient in need thereof a provided compound or a pharmaceutically acceptable salt or composition thereof.

In some embodiments the invention provides a method of treating a disease or condition commonly occurring in connection with transplantation. In some embodiments, the disease or condition commonly occurring in connection with transplantation is selected from organ transplantation, organ transplant rejection, and graft versus host disease.

In some embodiments the invention provides a method of treating a metabolic disease. In some embodiments the metabolic disease is selected from Type 1 diabetes, Type 2 diabetes, metabolic syndrome, and obesity.

In some embodiments the invention provides a method of treating a viral disease. In some embodiments, the viral infection is HIV or COVID19 infection.

In some embodiments, the present invention provides a method of treating a JAK-associated disease other than cancer.

Furthermore, the invention provides the use of a compound according to the definitions herein, or a pharmaceutically acceptable salt, or a hydrate or solvate thereof for the preparation of a medicament for the treatment of an inflammatory disease, an obstructive respiratory disease, a neurodegenerative disease, a viral disease, or a disorder commonly occurring in connection with transplantation.

### Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent. It will be appreciated that the present disclosure contemplates use of the combination therapies described herein only for treating the diseases, disorders, and conditions described herein.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif©), glatiramer acetate (Copaxone®), and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids agents that prolong or improve pharmacokinetics such as cytochrome P450 inhibitors (i.e., inhibitors of metabolic breakdown) and CYP3A4 inhibitors (e.g., ketokenozole and ritonavir), pirfenidone (Esbriet®), nintedanib (Ofev®), intravenous immunoglobulins, bosentan (Tracleer®), nifedipine (Procardia XL®), sildenafil (Revatio®), losartan (Cozaar®), iloprost (Ventavis®), topical nitroglycerin, N-acetylcysteine, antiacid therapy, and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen within greater than 24 hours apart.

In one embodiment, the present invention provides a composition comprising a provided compound and one or more additional therapeutic agents. The therapeutic agent may be administered together with a provided compound, or may be administered prior to or following administration of a provided compound. Suitable therapeutic agents are described in further detail below. In certain embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a provided compound and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-Bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating gout comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol and febuxostat (Uloric®).

In another embodiment, the present invention provides a method of treating rheumatoid arthritis comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating lupus comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating inflammatory bowel disease comprising administering to a patient in need thereof a provided compound and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a provided compound and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, the present invention provides a method of treating COPD comprising administering to a patient in need thereof a provided compound and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®.

In some embodiments, the present invention provides a method of treating HIV comprising administering to a patient in need thereof a provided compound and one or more additional therapeutic agents selected from nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), and combinations thereof.

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells. Other approved therapeutics that inhibit bone resorption include bisphosphonates, such as zoledronic acid (Zometa®, Novartis).

In some embodiments, the present invention provides a method of treating Alzheimer's disease comprising administering to a patient in need thereof a provided compound and one or more additional therapeutic agents selected from donepezil (Aricept®), rivastigmine (Excelon®), galantamine (Razadyne®), tacrine (Cognex®), and memantine (Namenda®).

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

Other kinase inhibitors and VEGF-R antagonists that are in development and may be used in the present invention include tivozanib (Aveo Pharmaceuticals); vatalanib (Bayer/Novartis); lucitanib (Clovis Oncology); dovitinib (TK1258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In another embodiment, the present invention provides a method of treating organ transplant rejection or graft vs. host disease comprising administering to a patient in need thereof a provided compound and one or more additional therapeutic agents selected from a steroid, cyclosporin, FK506, rapamycin, a hedgehog signaling inhibitor, a BTK inhibitor, a JAK/pan-JAK inhibitor, a TYK2 inhibitor, a PI3K inhibitor, and a SYK inhibitor.

In another embodiment, the present invention provides a method of treating or lessening the severity of a disease comprising administering to a patient in need thereof a provided compound and a BTK inhibitor, wherein the disease is selected from inflammatory bowel disease, arthritis, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, diseases of the bone and joints leading to joint inflammation and pain, cartilage and/or bone destruction, as well as bone regrowth and fusion, including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, idiopathic autoimmune hepatitis, gastritis, enteritis, dermatitis, gingivitis, appendicitis, pancreatitis, cholecystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenic purpura, myasthenia gravis, Hashimoto's thyroiditis, atopic dermatitis, degenerative joint disease, vitiligo, autoimmune hypopituitarism, Guillain-Barre syndrome, Behcet's disease, scleroderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a provided compound and a PI3K inhibitor, wherein the disease is selected from a neurodegenerative disorder, an angiogenic disorder, a viral disease, an autoimmune disease, an inflammatory disorder, conditions associated with organ transplantation, immunodeficiency disorders, an infectious disease, thrombin-induced platelet aggregation, liver disease, pathologic immune conditions involving T cell activation, and a cardiovascular disorder.

In another embodiment, the present invention provides a method of treating or lessening the severity of a disease comprising administering to a patient in need thereof a provided compound and a PI3K inhibitor, wherein the disease is selected from psoriasis or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced or exacerbated following bacterial or viral infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung disease or fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments, one or more other therapeutic agent is a phosphatidylinositol 3 kinase (PI3K) inhibitor. In some embodiments, a PI3K inhibitor is selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™.

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femara®, Novartis).

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™ Raloxifene hydrochloride is marketed under the trade name Evista™. Fulvestrant can be administered under the trade name Faslodex™.

The term “anti-androgen” as used herein relates to any substance which is capable of inhibiting the biological effects of androgenic hormones and includes, but is not limited to, bicalutamide (Casodex™) The term “gonadorelin agonist” as used herein includes, but is not limited to abarelix, goserelin and goserelin acetate. Goserelin can be administered under the trade name Zoladex™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS—354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

Compounds which target, decrease or inhibit the activity of a protein or lipid phosphatase are e.g. inhibitors of phosphatase 1, phosphatase 2A, or CDC25, such as okadaic acid or a derivative thereof.

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further anti-angiogenic compounds include compounds having another mechanism for their activity, e.g. unrelated to protein or lipid kinase inhibition e.g. thalidomide (Thalomid™) and TNP-470.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS—279251, BAY 12-9566, TAA211, MMI270B or AAJ996.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

Also included are EDG binders and ribonucleotide reductase inhibitors. The term “EDG binders” as used herein refers to a class of immunosuppressants that modulates lymphocyte recirculation, such as FTY720.

The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vemalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR—1, CCR—2, CCR—3, CCR—4, CCR—5, CCR—6, CCR—7, CCR—8, CCR—9 and CCR10, CXCR1, CXCR2, CXCR3, CXCR4, CXCR5, particularly CCR—5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of one or more other therapeutic agent present in the compositions of this invention may be no more than the amount that would normally be administered in a composition comprising that therapeutic agent as the only active agent. Preferably the amount of one or more other therapeutic agent in the presently disclosed compositions will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is provided for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

3b. Description of Exemplary Compound and Composition Embodiments

In certain embodiments, the present invention provides an inhibitor compound of formula I′:

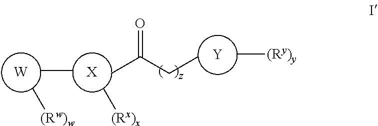

or a pharmaceutically acceptable salt thereof, wherein:
Ring W and its R^{w} substituents is

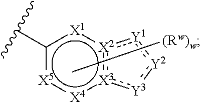

each of X¹, X⁴, and X⁵ is independently CH, CR^{w}, or N;
each of X² and X³ is independently C or N,
   wherein at most one of X¹, X², X³, X⁴, and X⁵ is N;
each of Y¹, Y², and Y³ is independently CH, CR^{w}, CH₂, CH(R^{w}), C(R^{w})₂, NH, NR^{w}, N, O, or S,
   wherein at least one of Y² and Y³ is CH, CR^{w}, CH₂, CH(R^{w}), or C(R^{w})₂;
each [symbol] represents a single bond or a double bond;
Ring X and its R^{x} substituents is

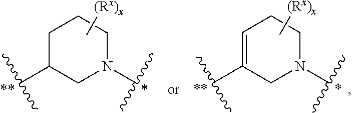

wherein

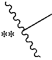

represents a bond to Ring W, and

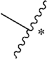

represents a bond to the rest of the molecule;
Ring Y is a ring selected from phenyl, 3-8 membered saturated or partially unsaturated monocyclic carbocyclyl or heterocyclyl with 1-3 heteroatoms independently selected from nitrogen, oxygen, and sulfur, and 5-6 membered monocyclic heteroaryl with 1-4 heteroatoms independently selected from nitrogen, oxygen and sulfur;
R^{w} is selected from hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
R^{w} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R^{B} is independently -L^{B}-Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, a 5-10 membered monocyclic or bicyclic arylenyl or heteroarylenyl having 1-4 heteroatoms independently selected from nitrogen, oxygen, and sulfur; or a 5-15 membered saturated or partially saturated monocyclic, bicyclic, or tricyclic carbocyclylenyl, heterocyclylenyl, arylenyl, or heteroarylenyl having 1-4 heteroatoms independently selected from nitrogen, oxygen, and sulfur;
each Cy^{B2} is independently an optionally substituted ring selected from phenyl, a 3-10 membered saturated or partially unsaturated monocyclic or bicyclic carbocyclic or heterocyclic ring having 1-2 heteroatoms independently selected from nitrogen, oxygen, and sulfur, a 5-10 membered monocyclic or bicyclic aryl or heteroaryl with 1-4 heteroatoms independently selected from nitrogen, oxygen and sulfur; or a 5-15 membered saturated or partially saturated monocyclic, bicyclic, or tricyclic carbocyclyl, heterocyclyl, aryl, or heteroaryl having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom or adjacent atoms are optionally taken together with their intervening atoms to form a 3-7 membered saturated or partially unsaturated ring having 0-3 heteroatoms, in addition to the atom or adjacent atoms to which they are attached, independently selected from nitrogen, oxygen, and sulfur; and
each of w, x, y, and z are independently 0, 1, 2, 3, or 4, and wherein:
   (i) when Ring X and its R^{x} substituents is

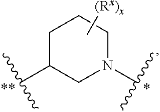

X¹ is N, and Y¹ is NH, NR^{w}, or N, then Y² and Y³ are not NH, NR^{w}, or N;
(ii) when Ring X and its R^{x} substituents is

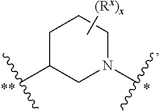

each of X¹, X⁴, and X⁵ is CH or CR^{w}, each of X² and X³ is C, and each of Y² and Y³ is CH, CR^{w}, CH₂, CH(R^{w}), or C(R^{w})₂, then Y¹ is not CH, CR^{w}, CH₂, CH(R^{w}), or C(R^{w})₂;
(iii) when Ring X and its R^{x} substituents is

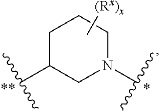

X¹ is N, and Y¹ is S, then Ring Y is not phenyl or 3-8 membered saturated or partially unsaturated monocyclic carbocyclyl or heterocyclyl with 1-3 heteroatoms independently selected from nitrogen, oxygen, and sulfur;
(iv) when Ring X and its R^{x} substituents is

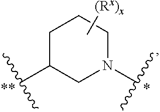

X¹ is N, and Y¹ is NH, NR^{w}, or N, then Y³ is not S;
(v) when Ring X and its R^{x} substituents is

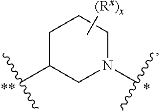

X¹ is N, and Y¹ is O, then Y² is not NH, NR^{w}, or N;
(vi) when Ring X and its R^{x} substituents is

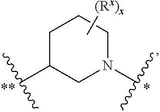

X¹ is N, Y¹ is NH, NR^{w}, or N, and Y² and Y³ are CH, CR^{w}, CH₂, CH(R^{w}), or C(R^{w})₂, then z is not 0 or 1;
(vii) when Ring X and its R^{x} substituents is

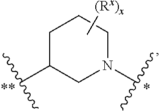

each of X¹, X⁴, and X⁵ is CH or CR^{w}, each of X² and X³ is C, and Y² is CH, CR^{w}, CH₂, CH(R^{w}), or C(R^{w})₂, then only one of Y² and Y³ is O;
(viii) when Ring X and its R^{x} substituents is

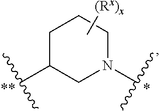

each of X¹, X⁴, and X⁵ is CH or CR^{w}, each of X² and X³ is C, Y¹ is O, NH, NR^{w}, or N, and Y² and Y³ are CH, CR^{w}, CH₂, CH(R^{w}), or C(R^{w})₂, then z is not 0 or 1; and
(ix) when Ring X and its R^{x} substituents is

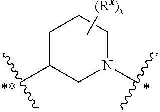

X⁵ is N, and Y¹ is NH, NR^{w}, or N, then Y² is not NH, NR^{w}, or N, and
wherein the inhibitor compound is not a compound of structure A-B-C defined by the combination of the building blocks A, B, and C within the table below:

| A | B | C |
|---|---|---|
| 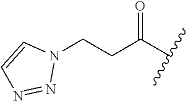 [SMILES: *C(=O)CCn1ccnn1] | 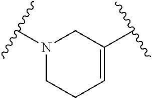 [SMILES: *C1=CCCN(*)C1] | 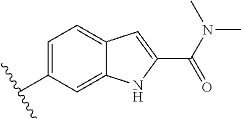 [SMILES: *c1ccc2cc(C(=O)N(C)C)[nH]c2c1] |
| 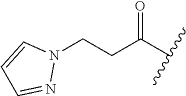 [SMILES: *C(=O)CCn1cccn1] | | 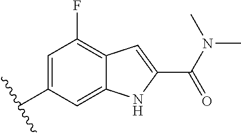 [SMILES: *c1cc(F)c2cc(C(=O)N(C)C)[nH]c2c1] |
| 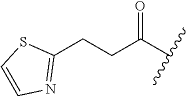 [SMILES: *C(=O)CCc1nccs1] | | 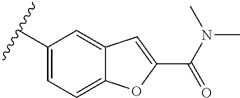 [SMILES: *c1ccc2oc(C(=O)N(C)C)cc2c1] |
| 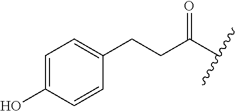 [SMILES: *C(=O)CCc1ccc(O)cc1] | | 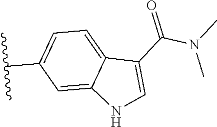 [SMILES: *c1ccc2c(C(=O)N(C)C)c[nH]c2c1] |
| 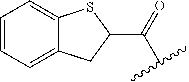 [SMILES: *C(=O)C1Cc2ccccc2S1] | | |

In some embodiments, the inhibitor compound of formula I′ specifically affects, as its primary mechanism of action, the inhibition of STAT6.

It will be understood that formula I′ cannot extend beyond the formulae described within this section (3b) below and herein. It will be understood that all references to “defined and described herein” solely refer to this section (3b) of the application. Additionally, all chemical formulae and embodiments within section 3b can only apply to and can be combined with formulae and embodiments from this section (3b).

In some embodiments, the inhibitor compound of formula I′ is not any of compounds I-1 through I-1548, or pharmaceutically acceptable salts thereof, described in PCT/US2024/044544. In some embodiments, the inhibitor compound of formula I, I′, II, III, or IV is not a compound of Table 1, or a pharmaceutically acceptable salt thereof, of International Application No. PCT/US2024/044544.

In some embodiments, the inhibitor compound of formula I′ is not a compound selected from Table B.

TABLE B

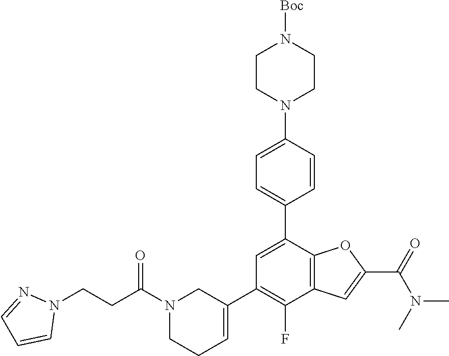 [SMILES: CN(C)C(=O)c1cc2c(F)c(C3=CCCN(C(=O)CCn4cccn4)C3)cc(-c3ccc(N4CCN(C(=O)OC(C)(C)C)CC4)cc3)c2o1]

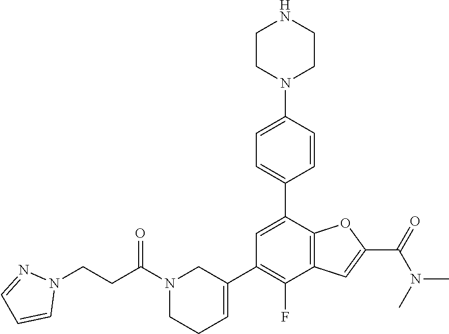 [SMILES: CN(C)C(=O)c1cc2c(F)c(C3=CCCN(C(=O)CCn4cccn4)C3)cc(-c3ccc(N4CCNCC4)cc3)c2o1]

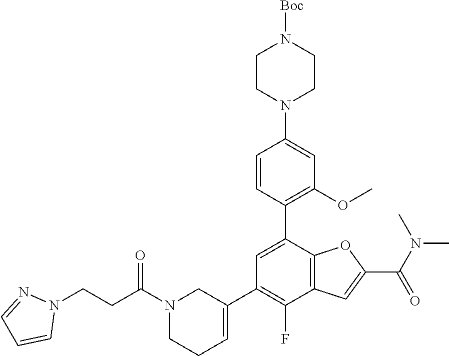 [SMILES: COc1cc(N2CCN(C(=O)OC(C)(C)C)CC2)ccc1-c1cc(C2=CCCN(C(=O)CCn3cccn3)C2)c(F)c2cc(C(=O)N(C)C)oc12]

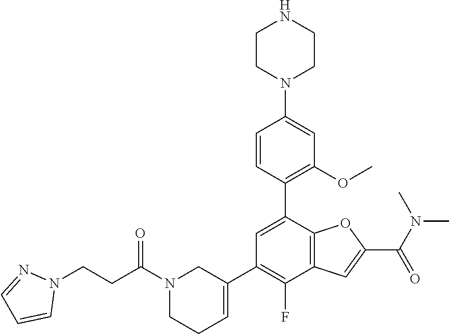 [SMILES: COc1cc(N2CCNCC2)ccc1-c1cc(C2=CCCN(C(=O)CCn3cccn3)C2)c(F)c2cc(C(=O)N(C)C)oc12]

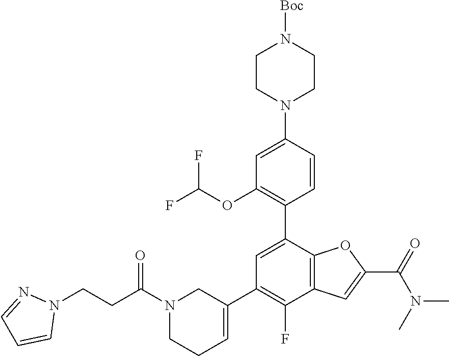 [SMILES: CN(C)C(=O)c1cc2c(F)c(C3=CCCN(C(=O)CCn4cccn4)C3)cc(-c3ccc(N4CCN(C(=O)OC(C)(C)C)CC4)cc3OC(F)F)c2o1]

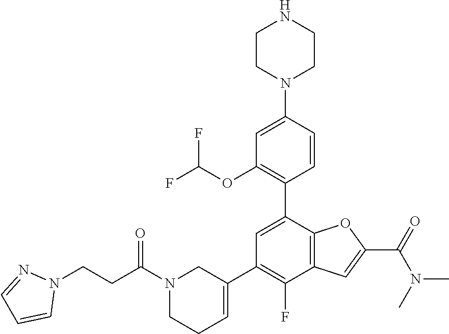 [SMILES: CN(C)C(=O)c1cc2c(F)c(C3=CCCN(C(=O)CCn4cccn4)C3)cc(-c3ccc(N4CCNCC4)cc3OC(F)F)c2o1]

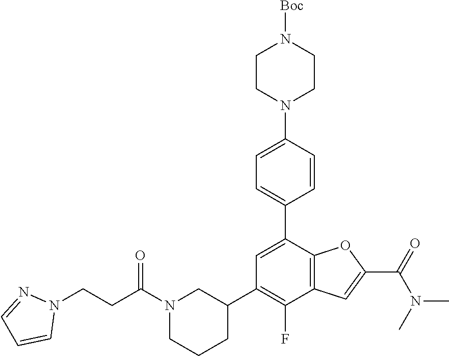 [SMILES: CN(C)C(=O)c1cc2c(F)c(C3CCCN(C(=O)CCn4cccn4)C3)cc(-c3ccc(N4CCN(C(=O)OC(C)(C)C)CC4)cc3)c2o1]

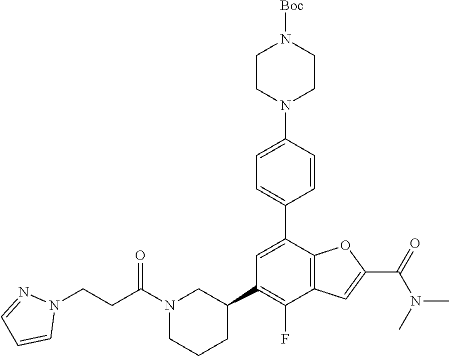 [SMILES: CN(C)C(=O)c1cc2c(F)c([C@H]3CCCN(C(=O)CCn4cccn4)C3)cc(-c3ccc(N4CCN(C(=O)OC(C)(C)C)CC4)cc3)c2o1]

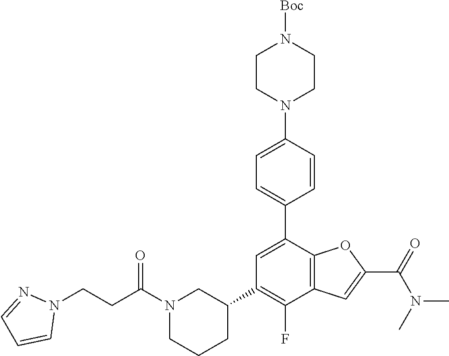 [SMILES: CN(C)C(=O)c1cc2c(F)c([C@@H]3CCCN(C(=O)CCn4cccn4)C3)cc(-c3ccc(N4CCN(C(=O)OC(C)(C)C)CC4)cc3)c2o1]

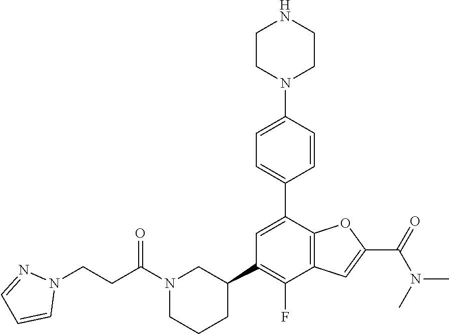 [SMILES: CN(C)C(=O)c1cc2c(F)c([C@H]3CCCN(C(=O)CCn4cccn4)C3)cc(-c3ccc(N4CCNCC4)cc3)c2o1]

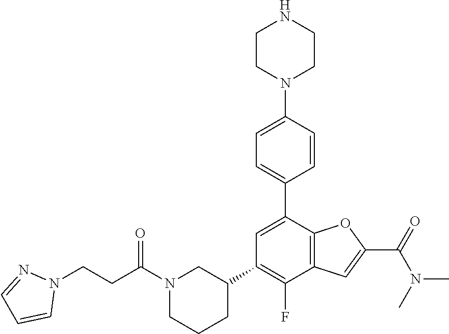

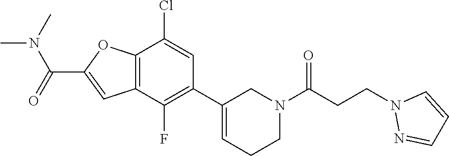 [SMILES: CN(C)C(=O)c1cc2c(F)c(C3=CCCN(C(=O)CCn4cccn4)C3)cc(Cl)c2o1]

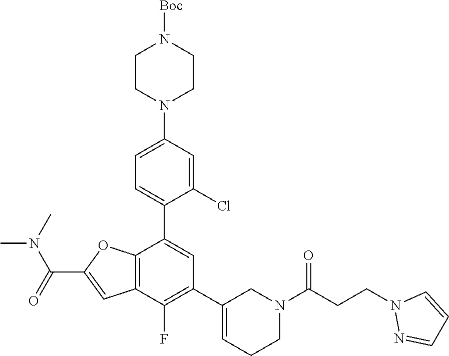 [SMILES: CN(C)C(=O)c1cc2c(F)c(C3=CCCN(C(=O)CCn4cccn4)C3)cc(-c3ccc(N4CCN(C(=O)OC(C)(C)C)CC4)cc3Cl)c2o1]

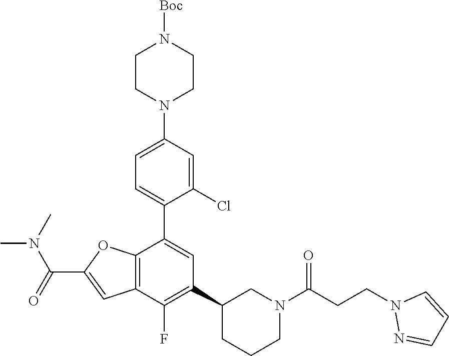 [SMILES: CN(C)C(=O)c1cc2c(F)c([C@@H]3CCCN(C(=O)CCn4cccn4)C3)cc(-c3ccc(N4CCN(C(=O)OC(C)(C)C)CC4)cc3Cl)c2o1]

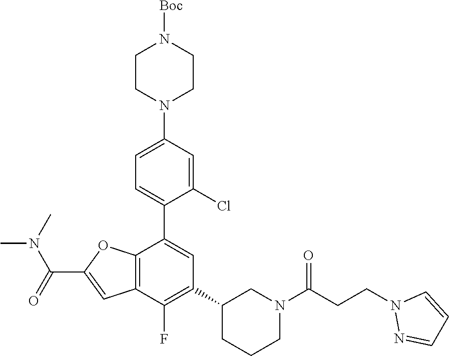 [SMILES: CN(C)C(=O)c1cc2c(F)c([C@H]3CCCN(C(=O)CCn4cccn4)C3)cc(-c3ccc(N4CCN(C(=O)OC(C)(C)C)CC4)cc3Cl)c2o1]

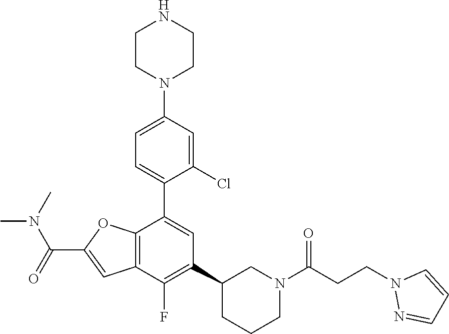 [SMILES: CN(C)C(=O)c1cc2c(F)c([C@@H]3CCCN(C(=O)CCn4cccn4)C3)cc(-c3ccc(N4CCNCC4)cc3Cl)c2o1]

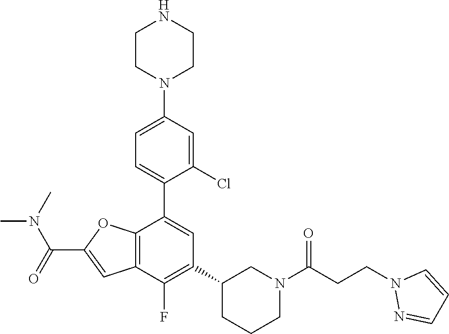 [SMILES: CN(C)C(=O)c1cc2c(F)c([C@H]3CCCN(C(=O)CCn4cccn4)C3)cc(-c3ccc(N4CCNCC4)cc3Cl)c2o1]

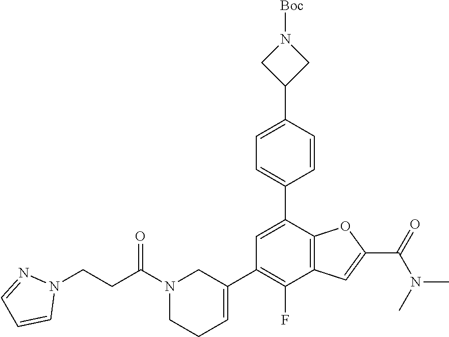 [SMILES: CN(C)C(=O)c1cc2c(F)c(C3=CCCN(C(=O)CCn4cccn4)C3)cc(-c3ccc(C4CN(C(=O)OC(C)(C)C)C4)cc3)c2o1]

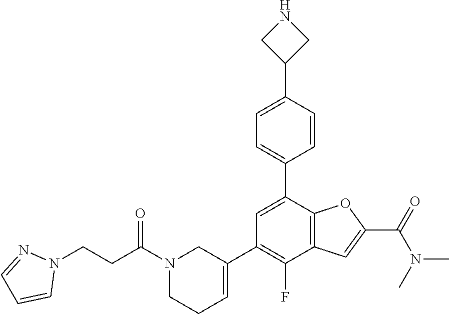 [SMILES: CN(C)C(=O)c1cc2c(F)c(C3=CCCN(C(=O)CCn4cccn4)C3)cc(-c3ccc(C4CNC4)cc3)c2o1]

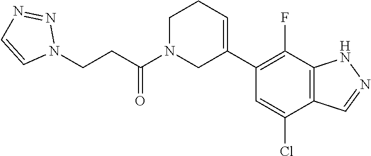 [SMILES: O=C(CCn1ccnn1)N1CCC=C(c2cc(Cl)c3cn[nH]c3c2F)C1]

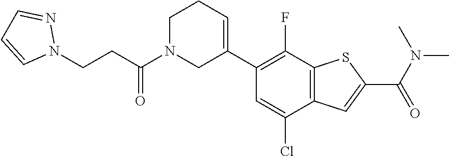 [SMILES: CN(C)C(=O)c1cc2c(Cl)cc(C3=CCCN(C(=O)CCn4cccn4)C3)c(F)c2s1]

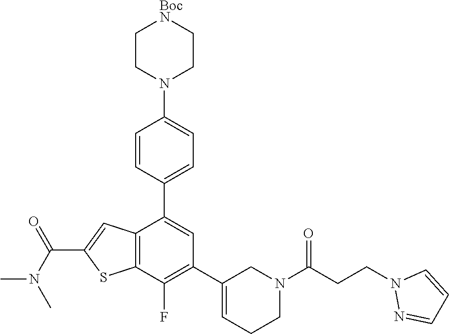 [SMILES: CN(C)C(=O)c1cc2c(-c3ccc(N4CCN(C(=O)OC(C)(C)C)CC4)cc3)cc(C3=CCCN(C(=O)CCn4cccn4)C3)c(F)c2s1]

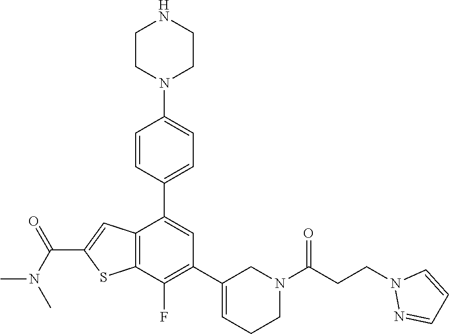 [SMILES: CN(C)C(=O)c1cc2c(-c3ccc(N4CCNCC4)cc3)cc(C3=CCCN(C(=O)CCn4cccn4)C3)c(F)c2s1]

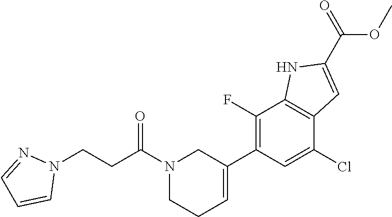 [SMILES: COC(=O)c1cc2c(Cl)cc(C3=CCCN(C(=O)CCn4cccn4)C3)c(F)c2[nH]1]

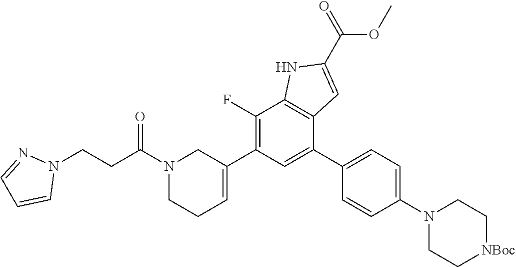 [SMILES: COC(=O)c1cc2c(-c3ccc(N4CCN(C(=O)OC(C)(C)C)CC4)cc3)cc(C3=CCCN(C(=O)CCn4cccn4)C3)c(F)c2[nH]1]

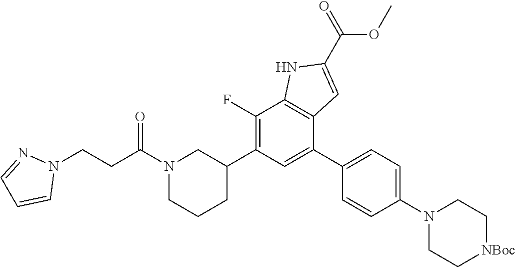 [SMILES: COC(=O)c1cc2c(-c3ccc(N4CCN(C(=O)OC(C)(C)C)CC4)cc3)cc(C3CCCN(C(=O)CCn4cccn4)C3)c(F)c2[nH]1]

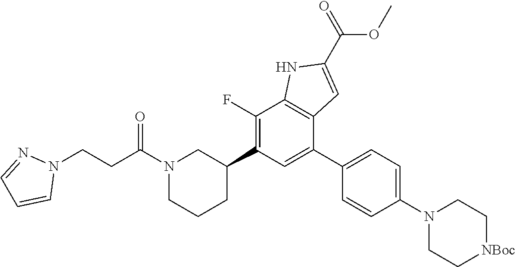 [SMILES: COC(=O)c1cc2c(-c3ccc(N4CCN(C(=O)OC(C)(C)C)CC4)cc3)cc([C@H]3CCCN(C(=O)CCn4cccn4)C3)c(F)c2[nH]1]

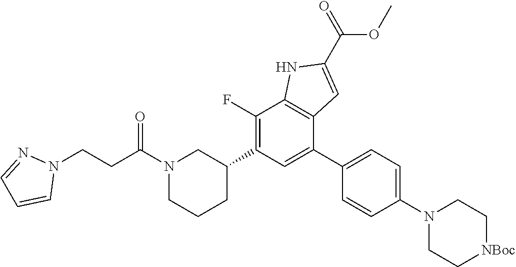 [SMILES: COC(=O)c1cc2c(-c3ccc(N4CCN(C(=O)OC(C)(C)C)CC4)cc3)cc([C@@H]3CCCN(C(=O)CCn4cccn4)C3)c(F)c2[nH]1]

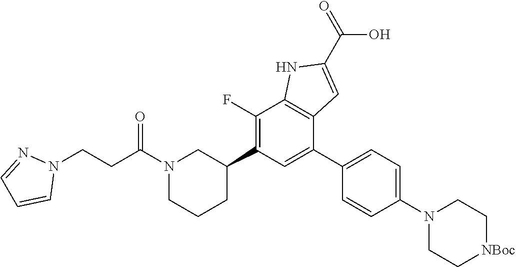 [SMILES: CC(C)(C)OC(=O)N1CCN(c2ccc(-c3cc([C@H]4CCCN(C(=O)CCn5cccn5)C4)c(F)c4[nH]c(C(=O)O)cc34)cc2)CC1]

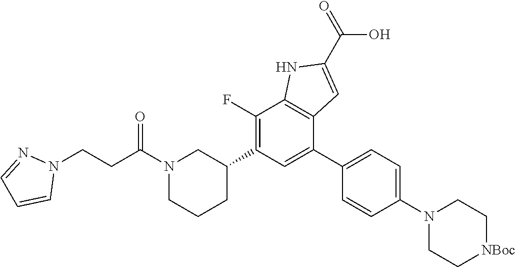 [SMILES: CC(C)(C)OC(=O)N1CCN(c2ccc(-c3cc([C@@H]4CCCN(C(=O)CCn5cccn5)C4)c(F)c4[nH]c(C(=O)O)cc34)cc2)CC1]

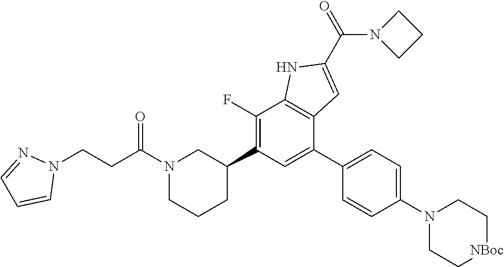 [SMILES: CC(C)(C)OC(=O)N1CCN(c2ccc(-c3cc([C@H]4CCCN(C(=O)CCn5cccn5)C4)c(F)c4[nH]c(C(=O)N5CCC5)cc34)cc2)CC1]

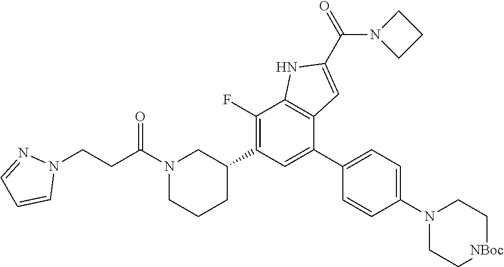 [SMILES: CC(C)(C)OC(=O)N1CCN(c2ccc(-c3cc([C@@H]4CCCN(C(=O)CCn5cccn5)C4)c(F)c4[nH]c(C(=O)N5CCC5)cc34)cc2)CC1]

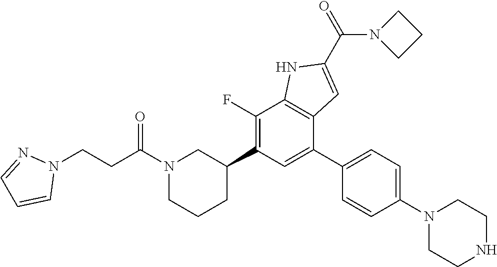 [SMILES: O=C(CCn1cccn1)N1CCC[C@H](c2cc(-c3ccc(N4CCNCC4)cc3)c3cc(C(=O)N4CCC4)[nH]c3c2F)C1]

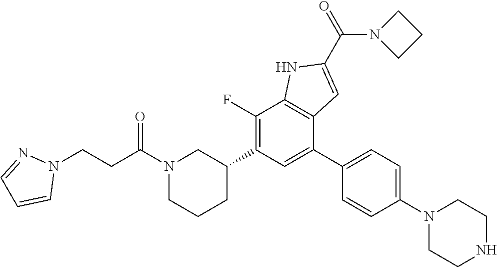 [SMILES: O=C(CCn1cccn1)N1CCC[C@@H](c2cc(-c3ccc(N4CCNCC4)cc3)c3cc(C(=O)N4CCC4)[nH]c3c2F)C1]

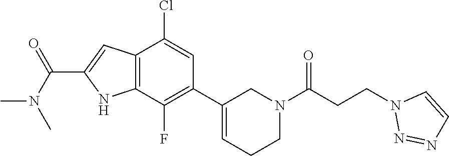 [SMILES: CN(C)C(=O)c1cc2c(Cl)cc(C3=CCCN(C(=O)CCn4ccnn4)C3)c(F)c2[nH]1]

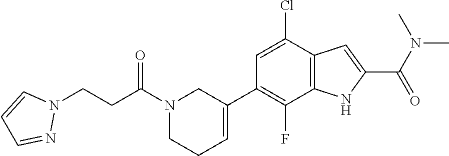 [SMILES: CN(C)C(=O)c1cc2c(Cl)cc(C3=CCCN(C(=O)CCn4cccn4)C3)c(F)c2[nH]1]

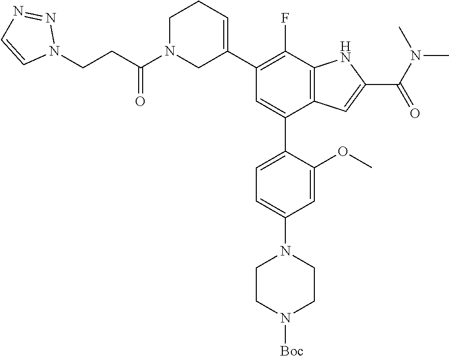 [SMILES: COc1cc(N2CCN(C(=O)OC(C)(C)C)CC2)ccc1-c1cc(C2=CCCN(C(=O)CCn3ccnn3)C2)c(F)c2[nH]c(C(=O)N(C)C)cc12]

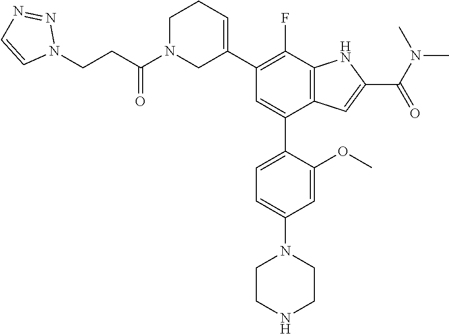 [SMILES: COc1cc(N2CCNCC2)ccc1-c1cc(C2=CCCN(C(=O)CCn3ccnn3)C2)c(F)c2[nH]c(C(=O)N(C)C)cc12]

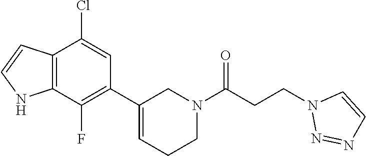 [SMILES: O=C(CCn1ccnn1)N1CCC=C(c2cc(Cl)c3cc[nH]c3c2F)C1]

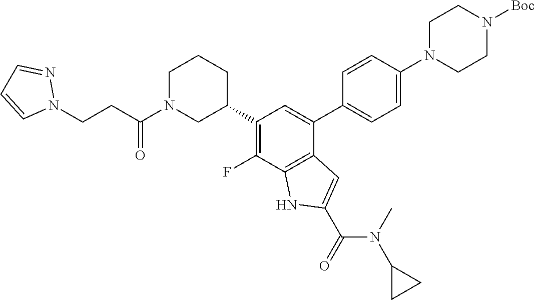 [SMILES: CN(C(=O)c1cc2c(-c3ccc(N4CCN(C(=O)OC(C)(C)C)CC4)cc3)cc([C@H]3CCCN(C(=O)CCn4cccn4)C3)c(F)c2[nH]1)C1CC1]

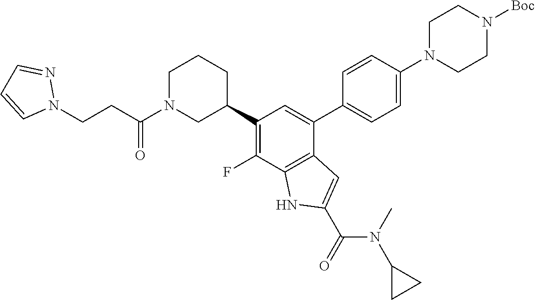 [SMILES: CN(C(=O)c1cc2c(-c3ccc(N4CCN(C(=O)OC(C)(C)C)CC4)cc3)cc([C@@H]3CCCN(C(=O)CCn4cccn4)C3)c(F)c2[nH]1)C1CC1]

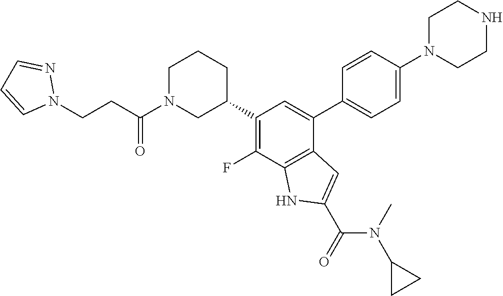 [SMILES: CN(C(=O)c1cc2c(-c3ccc(N4CCNCC4)cc3)cc([C@H]3CCCN(C(=O)CCn4cccn4)C3)c(F)c2[nH]1)C1CC1]

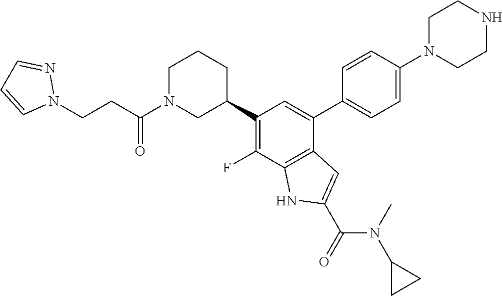 [SMILES: CN(C(=O)c1cc2c(-c3ccc(N4CCNCC4)cc3)cc([C@@H]3CCCN(C(=O)CCn4cccn4)C3)c(F)c2[nH]1)C1CC1]

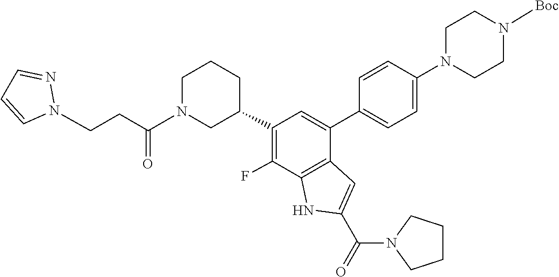 [SMILES: CC(C)(C)OC(=O)N1CCN(c2ccc(-c3cc([C@H]4CCCN(C(=O)CCn5cccn5)C4)c(F)c4[nH]c(C(=O)N5CCCC5)cc34)cc2)CC1]

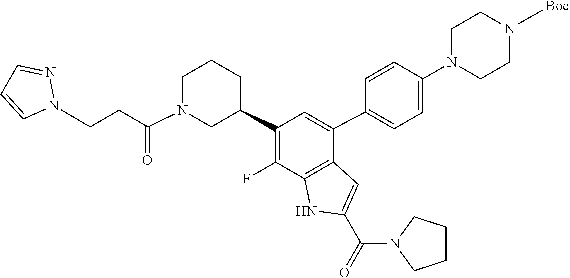 [SMILES: CC(C)(C)OC(=O)N1CCN(c2ccc(-c3cc([C@@H]4CCCN(C(=O)CCn5cccn5)C4)c(F)c4[nH]c(C(=O)N5CCCC5)cc34)cc2)CC1]

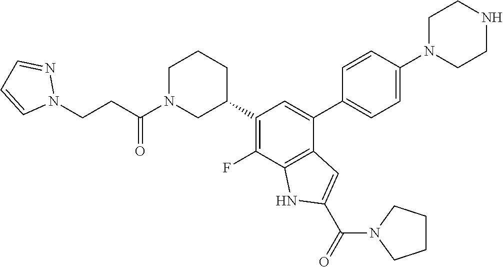 [SMILES: O=C(CCn1cccn1)N1CCC[C@H](c2cc(-c3ccc(N4CCNCC4)cc3)c3cc(C(=O)N4CCCC4)[nH]c3c2F)C1]

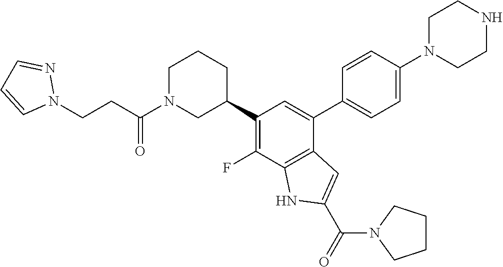 [SMILES: O=C(CCn1cccn1)N1CCC[C@@H](c2cc(-c3ccc(N4CCNCC4)cc3)c3cc(C(=O)N4CCCC4)[nH]c3c2F)C1]

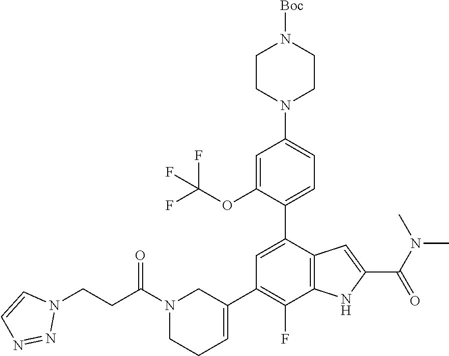 [SMILES: CN(C)C(=O)c1cc2c(-c3ccc(N4CCN(C(=O)OC(C)(C)C)CC4)cc3OC(F)(F)F)cc(C3=CCCN(C(=O)CCn4ccnn4)C3)c(F)c2[nH]1]

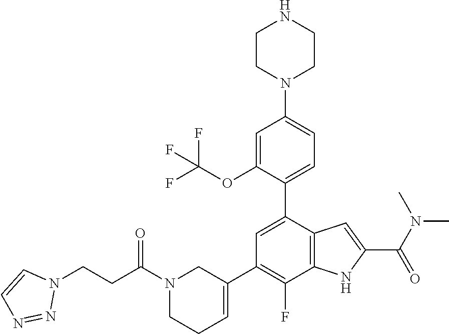 [SMILES: CN(C)C(=O)c1cc2c(-c3ccc(N4CCNCC4)cc3OC(F)(F)F)cc(C3=CCCN(C(=O)CCn4ccnn4)C3)c(F)c2[nH]1]

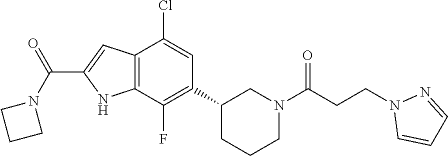 [SMILES: O=C(CCn1cccn1)N1CCC[C@H](c2cc(Cl)c3cc(C(=O)N4CCC4)[nH]c3c2F)C1]

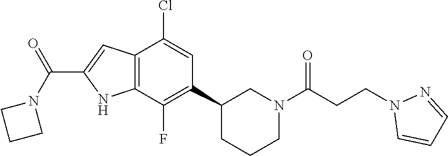 [SMILES: O=C(CCn1cccn1)N1CCC[C@@H](c2cc(Cl)c3cc(C(=O)N4CCC4)[nH]c3c2F)C1]

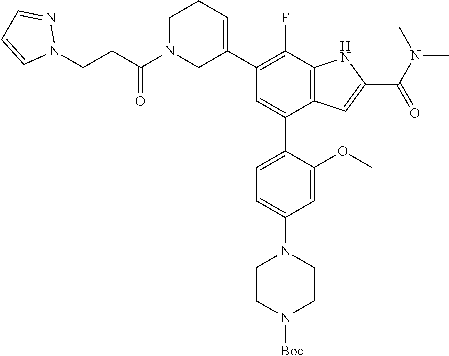 [SMILES: COc1cc(N2CCN(C(=O)OC(C)(C)C)CC2)ccc1-c1cc(C2=CCCN(C(=O)CCn3cccn3)C2)c(F)c2[nH]c(C(=O)N(C)C)cc12]

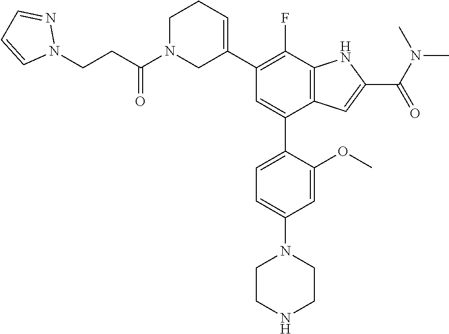 [SMILES: COc1cc(N2CCNCC2)ccc1-c1cc(C2=CCCN(C(=O)CCn3cccn3)C2)c(F)c2[nH]c(C(=O)N(C)C)cc12]

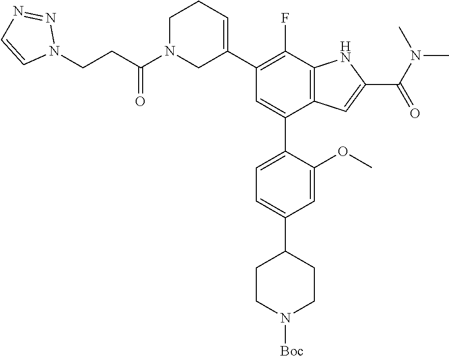 [SMILES: COc1cc(C2CCN(C(=O)OC(C)(C)C)CC2)ccc1-c1cc(C2=CCCN(C(=O)CCn3ccnn3)C2)c(F)c2[nH]c(C(=O)N(C)C)cc12]

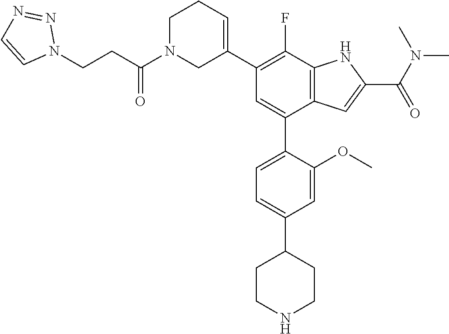 [SMILES: COc1cc(C2CCNCC2)ccc1-c1cc(C2=CCCN(C(=O)CCn3ccnn3)C2)c(F)c2[nH]c(C(=O)N(C)C)cc12]

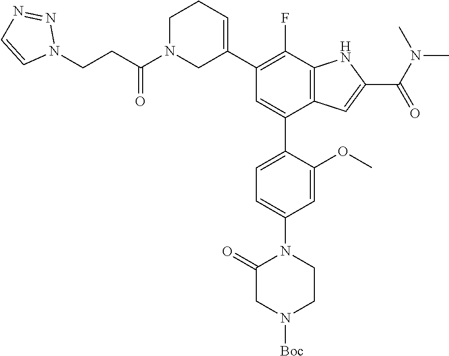 [SMILES: COc1cc(N2CCN(C(=O)OC(C)(C)C)CC2=O)ccc1-c1cc(C2=CCCN(C(=O)CCn3ccnn3)C2)c(F)c2[nH]c(C(=O)N(C)C)cc12]

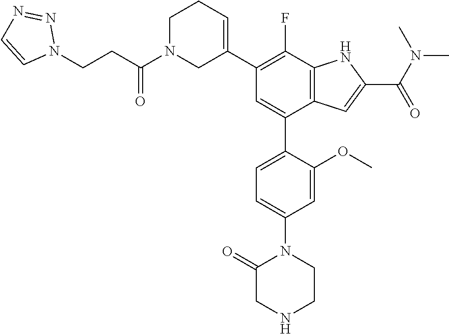 [SMILES: COc1cc(N2CCNCC2=O)ccc1-c1cc(C2=CCCN(C(=O)CCn3ccnn3)C2)c(F)c2[nH]c(C(=O)N(C)C)cc12]

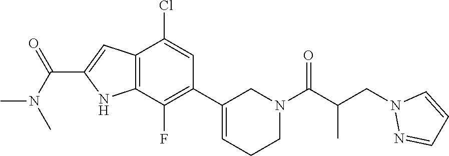 [SMILES: CC(Cn1cccn1)C(=O)N1CCC=C(c2cc(Cl)c3cc(C(=O)N(C)C)[nH]c3c2F)C1]

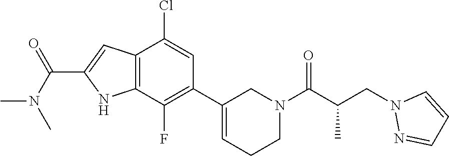 [SMILES: C[C@@H](Cn1cccn1)C(=O)N1CCC=C(c2cc(Cl)c3cc(C(=O)N(C)C)[nH]c3c2F)C1]

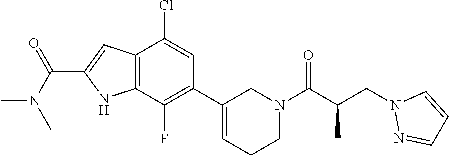 [SMILES: C[C@H](Cn1cccn1)C(=O)N1CCC=C(c2cc(Cl)c3cc(C(=O)N(C)C)[nH]c3c2F)C1]

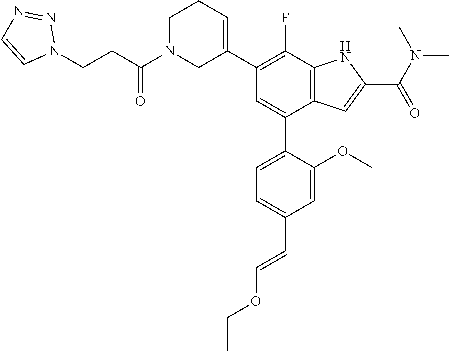 [SMILES: CCO/C=C/c1ccc(-c2cc(C3=CCCN(C(=O)CCn4ccnn4)C3)c(F)c3[nH]c(C(=O)N(C)C)cc23)c(OC)c1]

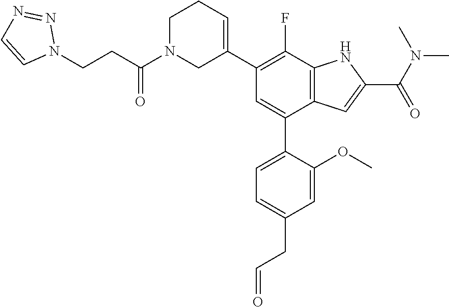 [SMILES: COc1cc(CC=O)ccc1-c1cc(C2=CCCN(C(=O)CCn3ccnn3)C2)c(F)c2[nH]c(C(=O)N(C)C)cc12]

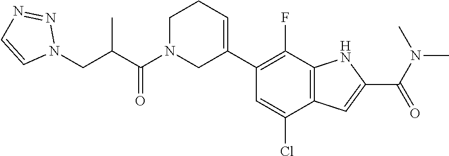 [SMILES: CC(Cn1ccnn1)C(=O)N1CCC=C(c2cc(Cl)c3cc(C(=O)N(C)C)[nH]c3c2F)C1]

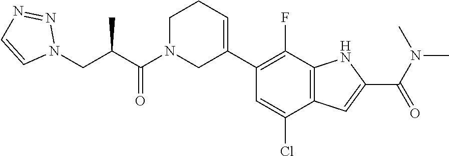 [SMILES: C[C@H](Cn1ccnn1)C(=O)N1CCC=C(c2cc(Cl)c3cc(C(=O)N(C)C)[nH]c3c2F)C1]

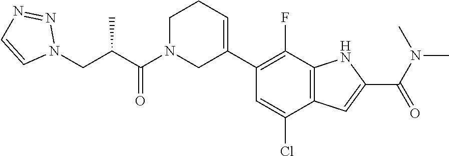 [SMILES: C[C@@H](Cn1ccnn1)C(=O)N1CCC=C(c2cc(Cl)c3cc(C(=O)N(C)C)[nH]c3c2F)C1]

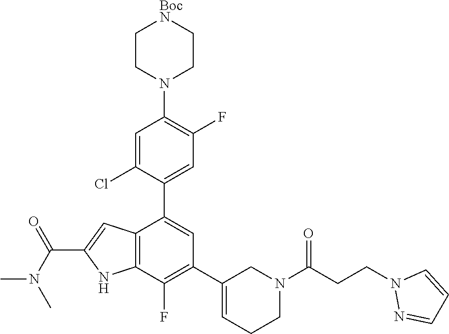 [SMILES: CN(C)C(=O)c1cc2c(-c3cc(F)c(N4CCN(C(=O)OC(C)(C)C)CC4)cc3Cl)cc(C3=CCCN(C(=O)CCn4cccn4)C3)c(F)c2[nH]1]

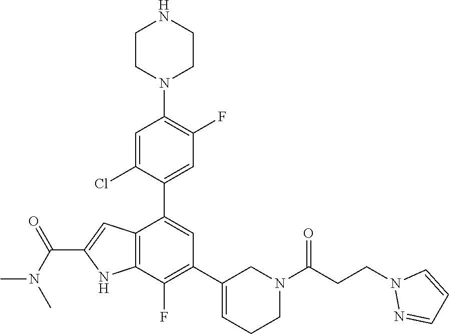 [SMILES: CN(C)C(=O)c1cc2c(-c3cc(F)c(N4CCNCC4)cc3Cl)cc(C3=CCCN(C(=O)CCn4cccn4)C3)c(F)c2[nH]1]

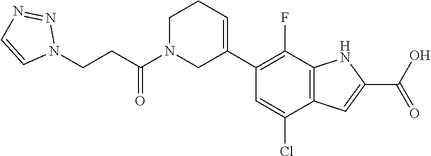 [SMILES: O=C(O)c1cc2c(Cl)cc(C3=CCCN(C(=O)CCn4ccnn4)C3)c(F)c2[nH]1]

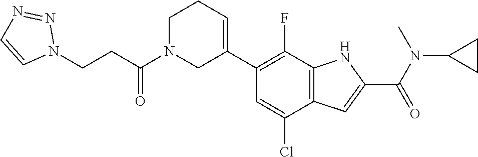 [SMILES: CN(C(=O)c1cc2c(Cl)cc(C3=CCCN(C(=O)CCn4ccnn4)C3)c(F)c2[nH]1)C1CC1]

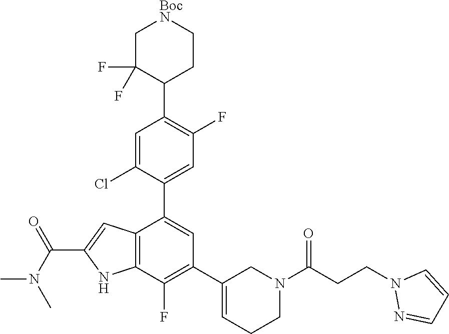 [SMILES: CN(C)C(=O)c1cc2c(-c3cc(F)c(C4CCN(C(=O)OC(C)(C)C)CC4(F)F)cc3Cl)cc(C3=CCCN(C(=O)CCn4cccn4)C3)c(F)c2[nH]1]

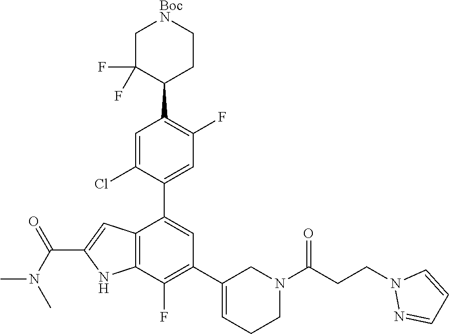 [SMILES: CN(C)C(=O)c1cc2c(-c3cc(F)c([C@@H]4CCN(C(=O)OC(C)(C)C)CC4(F)F)cc3Cl)cc(C3=CCCN(C(=O)CCn4cccn4)C3)c(F)c2[nH]1]

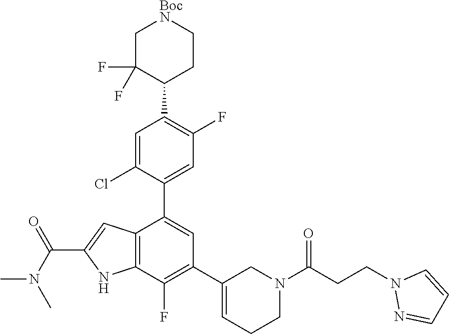 [SMILES: CN(C)C(=O)c1cc2c(-c3cc(F)c([C@H]4CCN(C(=O)OC(C)(C)C)CC4(F)F)cc3Cl)cc(C3=CCCN(C(=O)CCn4cccn4)C3)c(F)c2[nH]1]

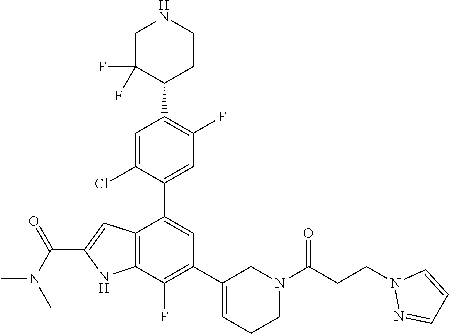 [SMILES: CN(C)C(=O)c1cc2c(-c3cc(F)c([C@H]4CCNCC4(F)F)cc3Cl)cc(C3=CCCN(C(=O)CCn4cccn4)C3)c(F)c2[nH]1]

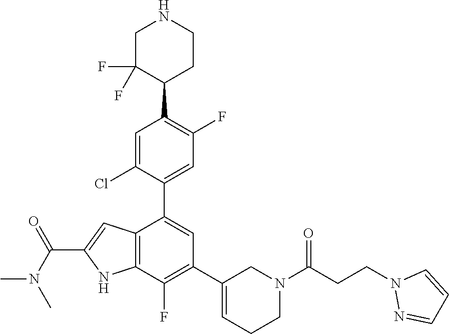 [SMILES: CN(C)C(=O)c1cc2c(-c3cc(F)c([C@@H]4CCNCC4(F)F)cc3Cl)cc(C3=CCCN(C(=O)CCn4cccn4)C3)c(F)c2[nH]1]

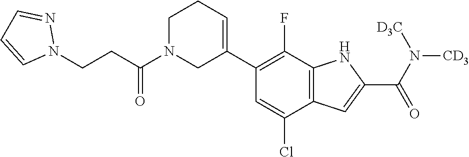 [SMILES: [2H]C([2H])([2H])N(C(=O)c1cc2c(Cl)cc(C3=CCCN(C(=O)CCn4cccn4)C3)c(F)c2[nH]1)C([2H])([2H])[2H]]

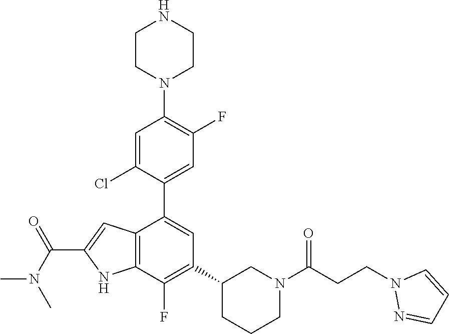 [SMILES: CN(C)C(=O)c1cc2c(-c3cc(F)c(N4CCNCC4)cc3Cl)cc([C@H]3CCCN(C(=O)CCn4cccn4)C3)c(F)c2[nH]1]

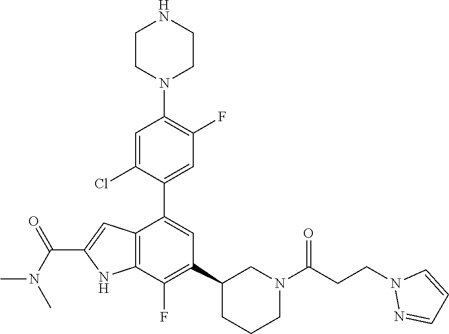 [SMILES: CN(C)C(=O)c1cc2c(-c3cc(F)c(N4CCNCC4)cc3Cl)cc([C@@H]3CCCN(C(=O)CCn4cccn4)C3)c(F)c2[nH]1]

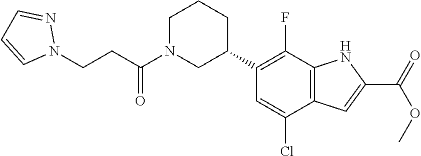 [SMILES: COC(=O)c1cc2c(Cl)cc([C@H]3CCCN(C(=O)CCn4cccn4)C3)c(F)c2[nH]1]

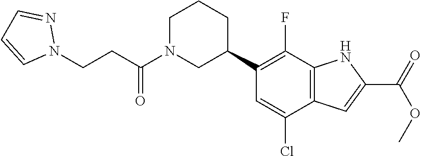 [SMILES: COC(=O)c1cc2c(Cl)cc([C@@H]3CCCN(C(=O)CCn4cccn4)C3)c(F)c2[nH]1]

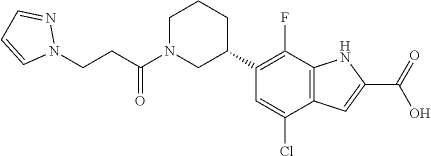 [SMILES: O=C(O)c1cc2c(Cl)cc([C@H]3CCCN(C(=O)CCn4cccn4)C3)c(F)c2[nH]1]

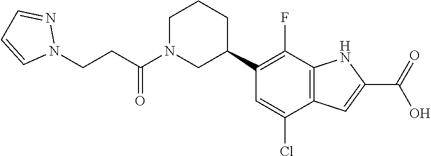 [SMILES: O=C(O)c1cc2c(Cl)cc([C@@H]3CCCN(C(=O)CCn4cccn4)C3)c(F)c2[nH]1]

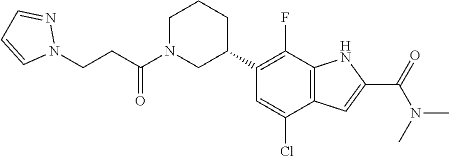 [SMILES: CN(C)C(=O)c1cc2c(Cl)cc([C@H]3CCCN(C(=O)CCn4cccn4)C3)c(F)c2[nH]1]

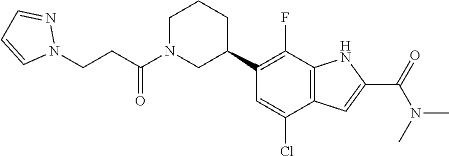 [SMILES: CN(C)C(=O)c1cc2c(Cl)cc([C@@H]3CCCN(C(=O)CCn4cccn4)C3)c(F)c2[nH]1]

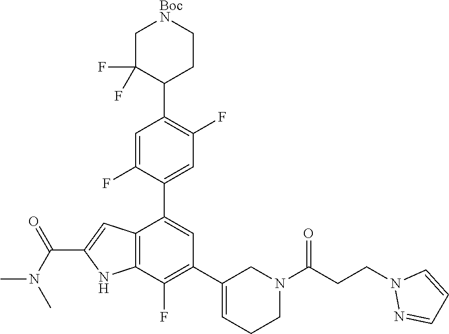 [SMILES: CN(C)C(=O)c1cc2c(-c3cc(F)c(C4CCN(C(=O)OC(C)(C)C)CC4(F)F)cc3F)cc(C3=CCCN(C(=O)CCn4cccn4)C3)c(F)c2[nH]1]

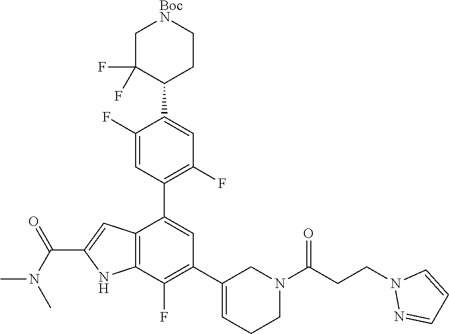 [SMILES: CN(C)C(=O)c1cc2c(-c3cc(F)c([C@H]4CCN(C(=O)OC(C)(C)C)CC4(F)F)cc3F)cc(C3=CCCN(C(=O)CCn4cccn4)C3)c(F)c2[nH]1]

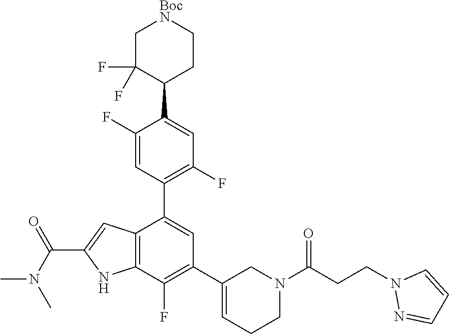 [SMILES: CN(C)C(=O)c1cc2c(-c3cc(F)c([C@@H]4CCN(C(=O)OC(C)(C)C)CC4(F)F)cc3F)cc(C3=CCCN(C(=O)CCn4cccn4)C3)c(F)c2[nH]1]

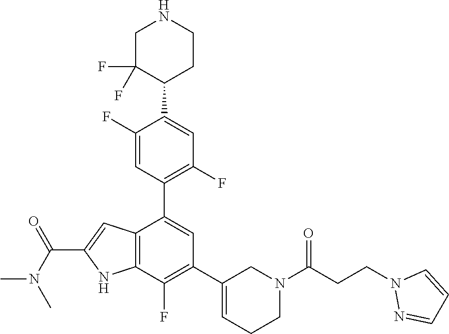 [SMILES: CN(C)C(=O)c1cc2c(-c3cc(F)c([C@H]4CCNCC4(F)F)cc3F)cc(C3=CCCN(C(=O)CCn4cccn4)C3)c(F)c2[nH]1]

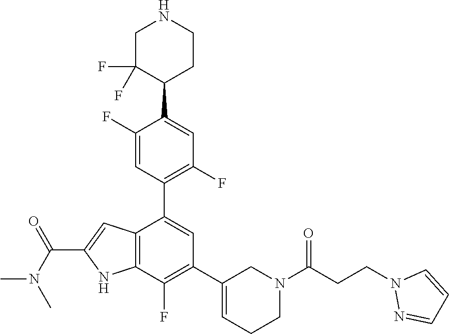 [SMILES: CN(C)C(=O)c1cc2c(-c3cc(F)c([C@@H]4CCNCC4(F)F)cc3F)cc(C3=CCCN(C(=O)CCn4cccn4)C3)c(F)c2[nH]1]

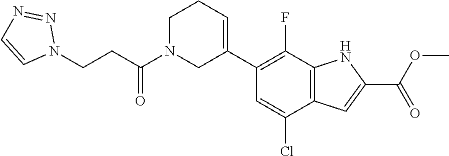 [SMILES: COC(=O)c1cc2c(Cl)cc(C3=CCCN(C(=O)CCn4ccnn4)C3)c(F)c2[nH]1]

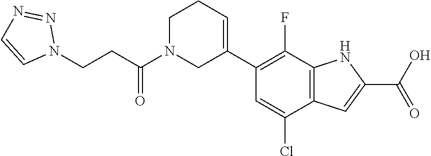 [SMILES: O=C(O)c1cc2c(Cl)cc(C3=CCCN(C(=O)CCn4ccnn4)C3)c(F)c2[nH]1]

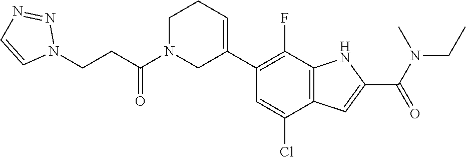 [SMILES: CCN(C)C(=O)c1cc2c(Cl)cc(C3=CCCN(C(=O)CCn4ccnn4)C3)c(F)c2[nH]1]

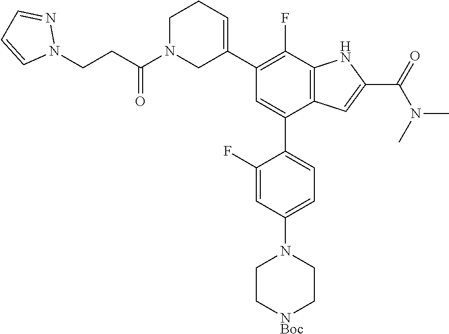 [SMILES: CN(C)C(=O)c1cc2c(-c3ccc(N4CCN(C(=O)OC(C)(C)C)CC4)cc3F)cc(C3=CCCN(C(=O)CCn4cccn4)C3)c(F)c2[nH]1]

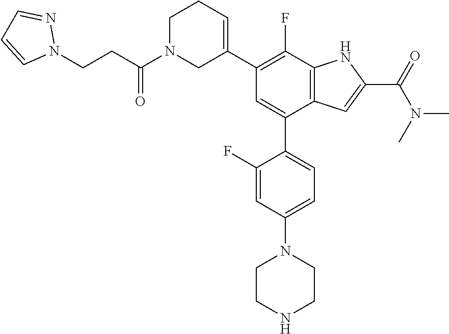 [SMILES: CN(C)C(=O)c1cc2c(-c3ccc(N4CCNCC4)cc3F)cc(C3=CCCN(C(=O)CCn4cccn4)C3)c(F)c2[nH]1]

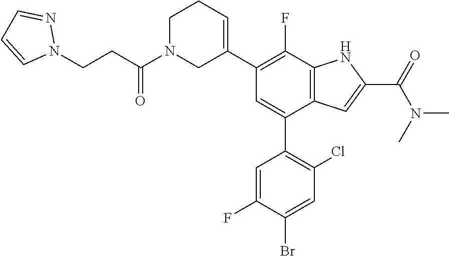 [SMILES: CN(C)C(=O)c1cc2c(-c3cc(F)c(Br)cc3Cl)cc(C3=CCCN(C(=O)CCn4cccn4)C3)c(F)c2[nH]1]

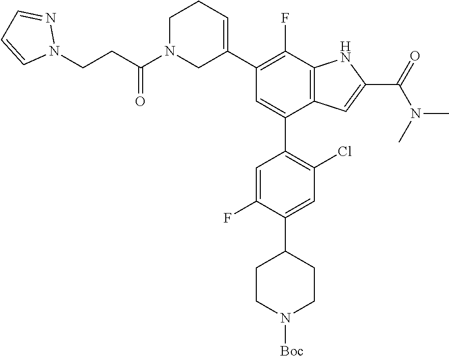 [SMILES: CN(C)C(=O)c1cc2c(-c3cc(F)c(C4CCN(C(=O)OC(C)(C)C)CC4)cc3Cl)cc(C3=CCCN(C(=O)CCn4cccn4)C3)c(F)c2[nH]1]

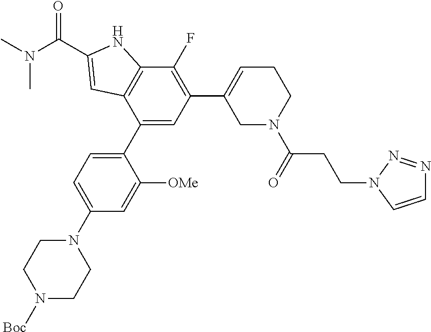 [SMILES: COc1cc(N2CCN(C(=O)OC(C)(C)C)CC2)ccc1-c1cc(C2=CCCN(C(=O)CCn3ccnn3)C2)c(F)c2[nH]c(C(=O)N(C)C)cc12]

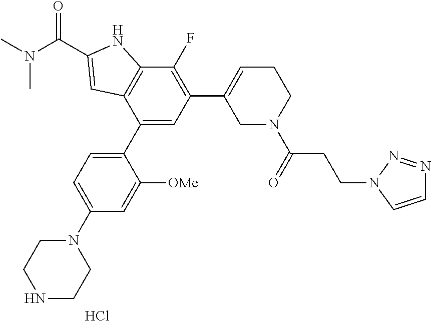 [SMILES: COc1cc(N2CCNCC2)ccc1-c1cc(C2=CCCN(C(=O)CCn3ccnn3)C2)c(F)c2[nH]c(C(=O)N(C)C)cc12 ; Cl]

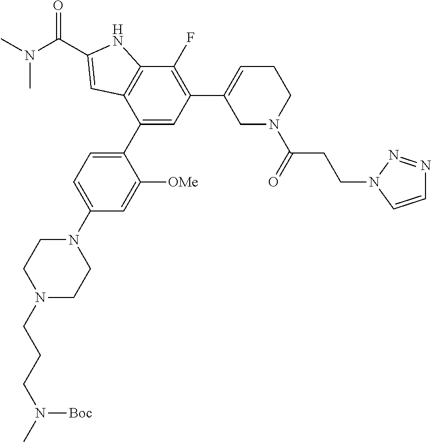 [SMILES: COc1cc(N2CCN(CCCN(C)C(=O)OC(C)(C)C)CC2)ccc1-c1cc(C2=CCCN(C(=O)CCn3ccnn3)C2)c(F)c2[nH]c(C(=O)N(C)C)cc12]

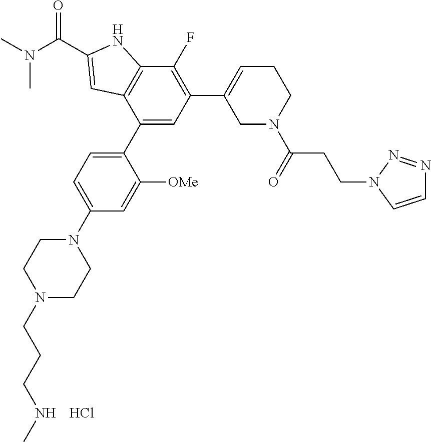 [SMILES: CNCCCN1CCN(c2ccc(-c3cc(C4=CCCN(C(=O)CCn5ccnn5)C4)c(F)c4[nH]c(C(=O)N(C)C)cc34)c(OC)c2)CC1 ; Cl]

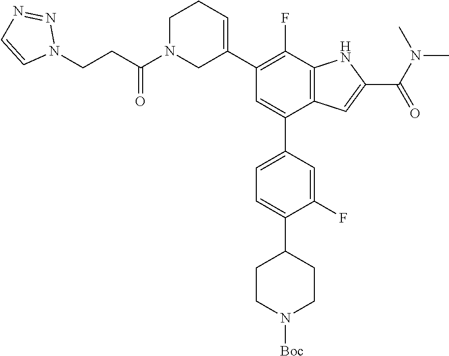 [SMILES: CN(C)C(=O)c1cc2c(-c3ccc(C4CCN(C(=O)OC(C)(C)C)CC4)c(F)c3)cc(C3=CCCN(C(=O)CCn4ccnn4)C3)c(F)c2[nH]1]

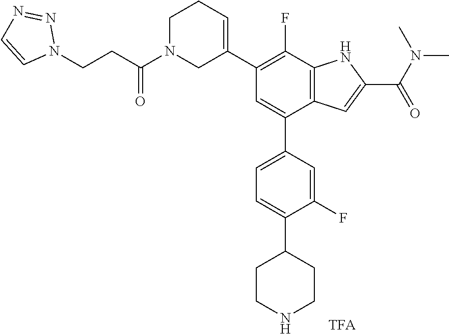 [SMILES: CN(C)C(=O)c1cc2c(-c3ccc(C4CCNCC4)c(F)c3)cc(C3=CCCN(C(=O)CCn4ccnn4)C3)c(F)c2[nH]1]

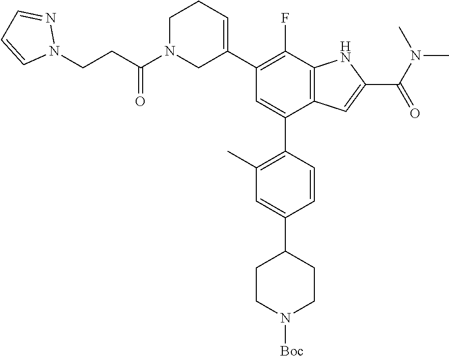 [SMILES: Cc1cc(C2CCN(C(=O)OC(C)(C)C)CC2)ccc1-c1cc(C2=CCCN(C(=O)CCn3cccn3)C2)c(F)c2[nH]c(C(=O)N(C)C)cc12]

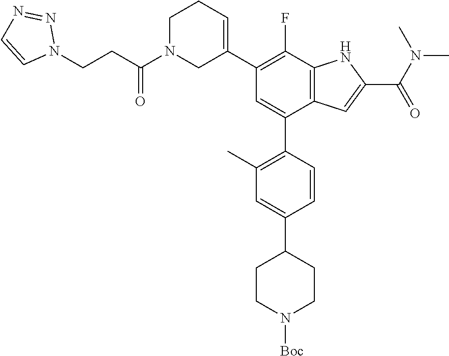 [SMILES: Cc1cc(C2CCN(C(=O)OC(C)(C)C)CC2)ccc1-c1cc(C2=CCCN(C(=O)CCn3ccnn3)C2)c(F)c2[nH]c(C(=O)N(C)C)cc12]

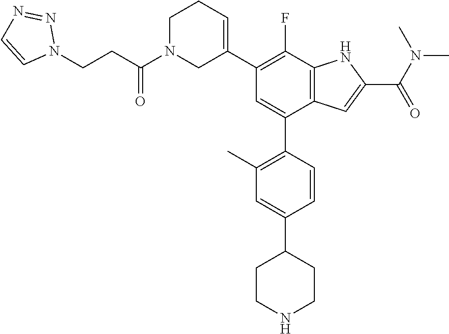 [SMILES: Cc1cc(C2CCNCC2)ccc1-c1cc(C2=CCCN(C(=O)CCn3ccnn3)C2)c(F)c2[nH]c(C(=O)N(C)C)cc12]

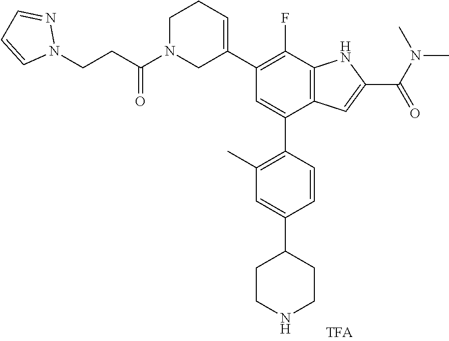 [SMILES: Cc1cc(C2CCNCC2)ccc1-c1cc(C2=CCCN(C(=O)CCn3cccn3)C2)c(F)c2[nH]c(C(=O)N(C)C)cc12]

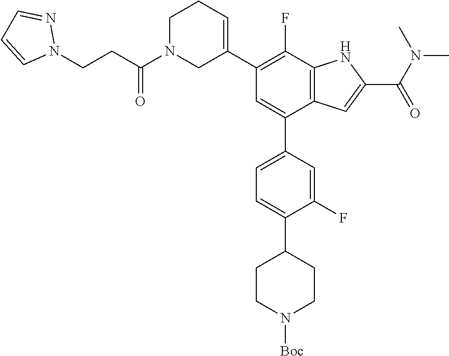 [SMILES: CN(C)C(=O)c1cc2c(-c3ccc(C4CCN(C(=O)OC(C)(C)C)CC4)c(F)c3)cc(C3=CCCN(C(=O)CCn4cccn4)C3)c(F)c2[nH]1]

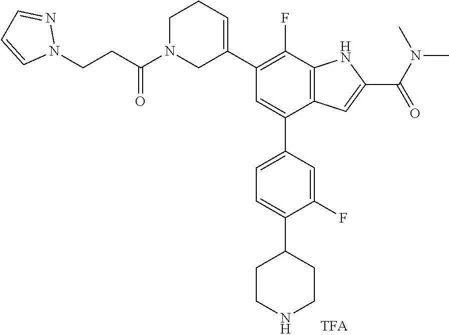 [SMILES: CN(C)C(=O)c1cc2c(-c3ccc(C4CCNCC4)c(F)c3)cc(C3=CCCN(C(=O)CCn4cccn4)C3)c(F)c2[nH]1]

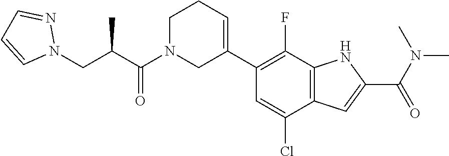 [SMILES: C[C@H](Cn1cccn1)C(=O)N1CCC=C(c2cc(Cl)c3cc(C(=O)N(C)C)[nH]c3c2F)C1]

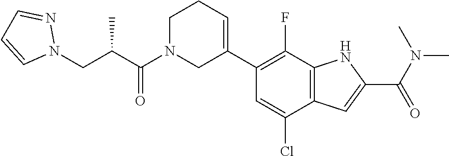 [SMILES: C[C@@H](Cn1cccn1)C(=O)N1CCC=C(c2cc(Cl)c3cc(C(=O)N(C)C)[nH]c3c2F)C1]

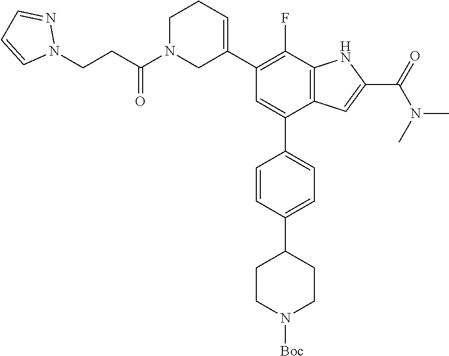 [SMILES: CN(C)C(=O)c1cc2c(-c3ccc(C4CCN(C(=O)OC(C)(C)C)CC4)cc3)cc(C3=CCCN(C(=O)CCn4cccn4)C3)c(F)c2[nH]1]

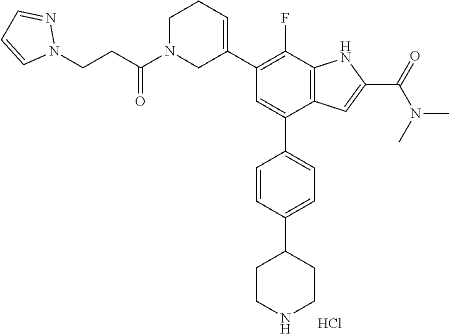 [SMILES: CN(C)C(=O)c1cc2c(-c3ccc(C4CCNCC4)cc3)cc(C3=CCCN(C(=O)CCn4cccn4)C3)c(F)c2[nH]1 ; Cl]

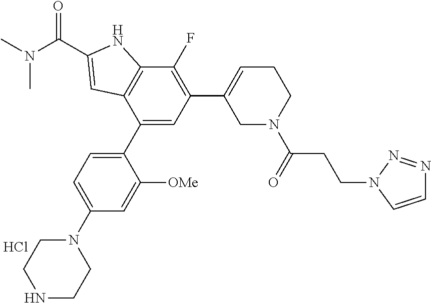 [SMILES: COc1cc(N2CCNCC2)ccc1-c1cc(C2=CCCN(C(=O)CCn3ccnn3)C2)c(F)c2[nH]c(C(=O)N(C)C)cc12 ; Cl]

or a salt or free base/acid thereof.

As described above and defined herein, Ring W and its R^{w} substituents is

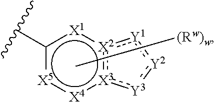

wherein each of X¹, X⁴, and X⁵ is independently CH, CR^{w}, or N; each of X² and X³ is independently C or N, wherein at most one of X¹, X², X³, X⁴, and X⁵ is N; each of Y¹, Y², and Y³ is independently CH, CR^{w}, CH₂, CH(R^{w}), C(R^{w})₂, NH, NR^{w}, N, O, or S, wherein at least one of Y² and Y³ is CH, CR^{w}, CH₂, CH(R^{w}), or C(R^{w})₂; and each [symbol] represents a single bond or a double bond.

In some embodiments, the ring containing X², X³, Y¹, Y², and Y³ is aromatic or heteroaromatic, i.e., the definitions of Y¹, Y², Y³, and [symbol] are selected to satisfy valency of an aromatic or heteroaromatic ring. In some embodiments, the ring-containing X², X³, Y¹, Y², and Y³ is not aromatic or heteroaromatic, i.e., the definitions of Y¹, Y², Y³, and [symbol] are selected to satisfy valency of a partially unsaturated ring.

In some embodiments, Ring W and its R^{w} substituents is:

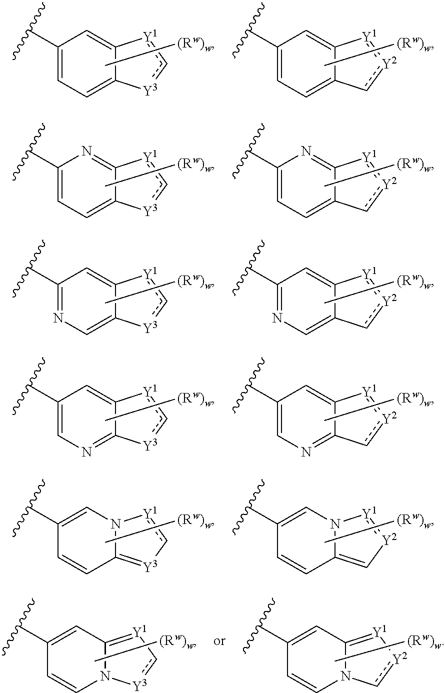

As defined above and described herein, each of X¹, X⁴, and X⁵ is independently CH, CR^{w}, or N. In some embodiments, each of X¹, X⁴, and X⁵ is CH or CR^{w}. In some embodiments, X¹ is CH. In some embodiments, X¹ is CR^{w}. In some embodiments, X¹ is N. In some embodiments, X⁴ is CH. In some embodiments, X⁴ is CR^{w}. In some embodiments, X⁴ is N. In some embodiments, X⁵ is CH. In some embodiments, X⁵ is CR^{w}. In some embodiments, X⁵ is N.

As defined above and described herein, each of X² and X³ is independently C or N. In some embodiments, both of X² and X³ are C. In some embodiments, X² is C. In some embodiments, X² is N. In some embodiments, X³ is C. In some embodiments, X³ is N.

In some embodiments, X¹, X⁴, and X⁵ are CH or CR^{w}, and X² and X³ are C. In some embodiments, X¹ is N, X² and X³ are C, and X⁴ and X⁵ are CH or CR^{w}. In some embodiments, X¹, X⁴, and X⁵ are CH or CR^{w}, X² is N, and X³ are C. In some embodiments, X¹, X⁴, and X⁵ are CH or CR^{w}, X² is C, and X³ are N. In some embodiments, X¹ and X⁵ are CH or CR^{w}, X² and X³ are C, and X⁴ is N. In some embodiments, X¹ and X⁴ are CH or CR^{w}, X² and X³ are C, and X⁵ is N.

As defined above and described herein, each of Y¹, Y², and Y³ is independently CH, CR^{w}, CH₂, CH(R^{w}), C(R^{w})₂, NH, NR^{w}, N, O, or S, wherein at least one of Y² and Y³ is CH, CR^{w}, CH₂, CH(R^{w}), or C(R^{w})₂. In some embodiments, both of Y² and Y³ are CH, CR^{w}, CH₂, CH(R^{w}), or C(R^{w})₂. In some embodiments, each of Y¹, Y², and Y³ is independently CH, CR^{w}, NH, NR^{w}, N, O, or S, wherein at least one of Y² and Y³ is CH or CR^{w}. In some embodiments, Y¹ is CH. In some embodiments, Y¹ is CR^{w}. In some embodiments, Y¹ is CH₂. In some embodiments, Y¹ is CH(R^{w}). In some embodiments, Y¹ is C(R^{w})₂. In some embodiments, Y¹ is NH. In some embodiments, Y¹ is NR^{w}. In some embodiments, Y¹ is N. In some embodiments, Y¹ is O. In some embodiments, Y¹ is S. In some embodiments, Y² is CH. In some embodiments, Y² is CR^{w}. In some embodiments, Y² is CH₂. In some embodiments, Y² is CH(R^{w}). In some embodiments, Y² is C(R^{w})₂. In some embodiments, Y² is NH. In some embodiments, Y² is NR^{w}. In some embodiments, Y² is N. In some embodiments, Y² is O. In some embodiments, Y² is S. In some embodiments, Y³ is CH. In some embodiments, Y³ is CR^{w}. In some embodiments, Y³ is CH₂. In some embodiments, Y³ is CH(R^{w}). In some embodiments, Y³ is C(R^{w})₂. In some embodiments, Y³ is NH. In some embodiments, Y³ is NR^{w}. In some embodiments, Y³ is N. In some embodiments, Y³ is O. In some embodiments, Y³ is S.

In some embodiments, Y¹ is NH or NR^{w}, and Y² and Y³ are CH or CR^{w}. In some embodiments, Y¹ is O, and Y² and Y³ are CH or CR^{w}. In some embodiments, Y¹ is S, and Y² and Y³ are CH or CR^{w}.

In some embodiments, Ring W and its R^{w} substituents is

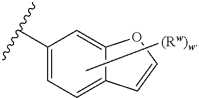

In some embodiments, Ring W and its R^{w} substituents is

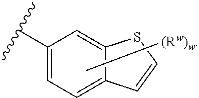

In some embodiments, Ring W and its R^{w} substituents is

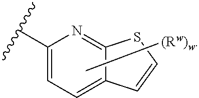

In some embodiments, Ring W and its R^{w} substituents is

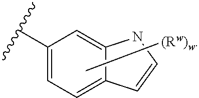

In some embodiments, Ring W and its R^{w} substituents is

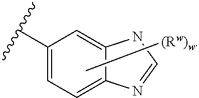

In some embodiments, Ring W and its R^{w} substituents is

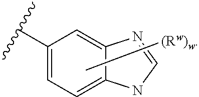

In some embodiments, Ring W and its R^{w} substituents is

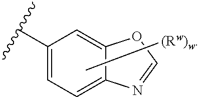

In some embodiments, Ring W and its R^{w} substituents is

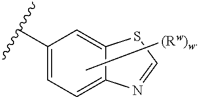

In some embodiments, Ring W and its R^{w} substituents is

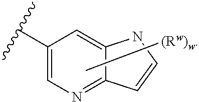

In some embodiments, Ring W and its R^{w} substituents is

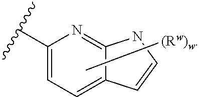

In some embodiments, Ring W and its R^{w} substituents is

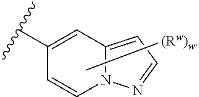

In some embodiments, Ring W and its R^{w} substituents is

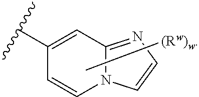

In some embodiments, Ring W and its R^{w} substituents is

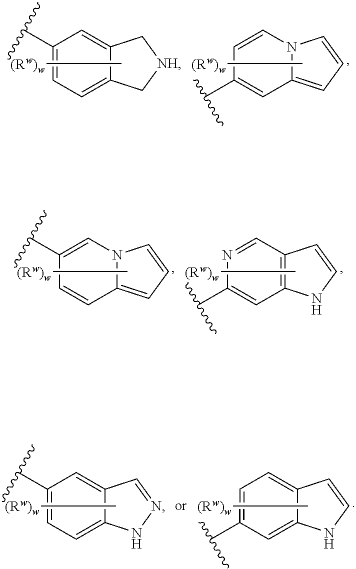

In some embodiments, Ring W and its R^{w} substituents is

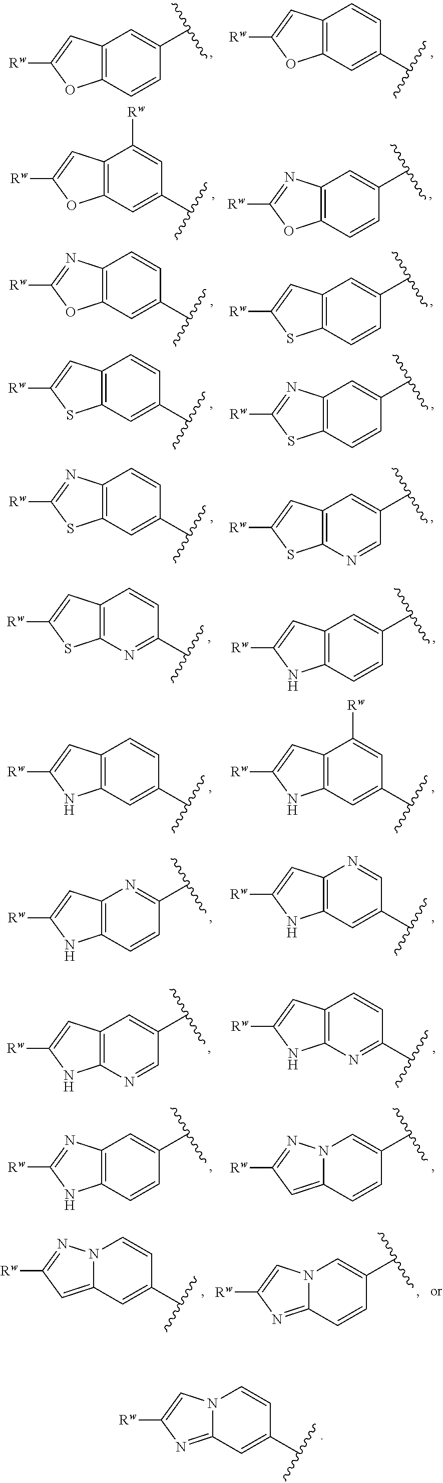

In some embodiments, Ring W and its R^{w} substituents is

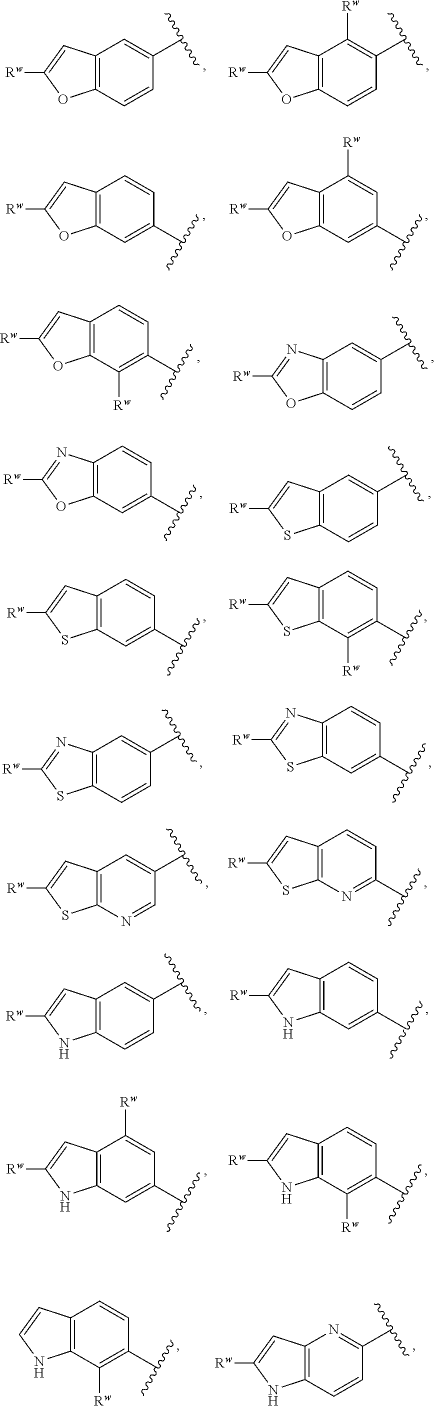

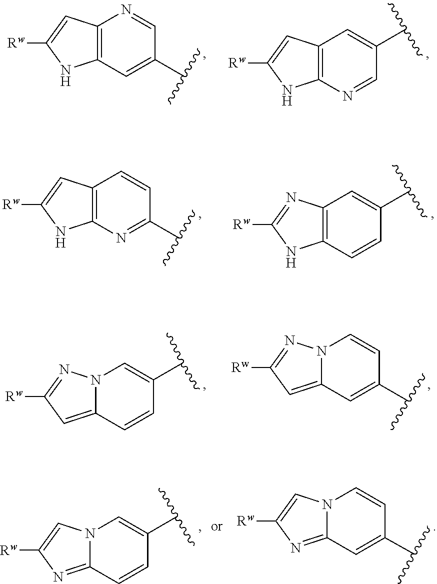

In some embodiments, Ring W and its R^{w} substituents is

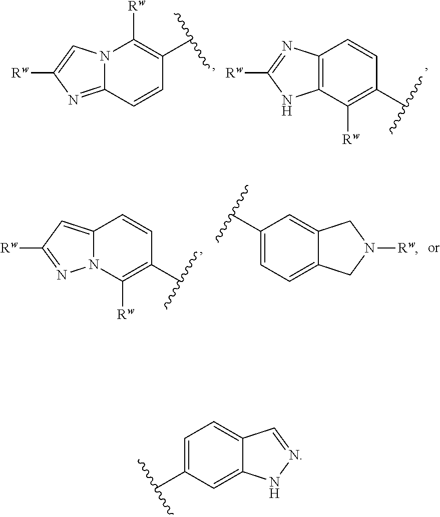

In some embodiments, Ring W and its R^{w} substituents is

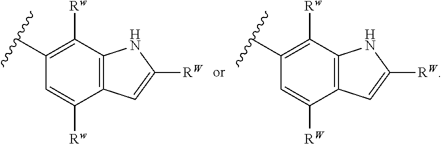

In some embodiments Ring W and its R^{w} substituents is

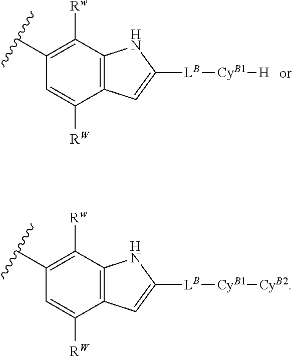

In some embodiments Ring W and its R^{w} substituents is

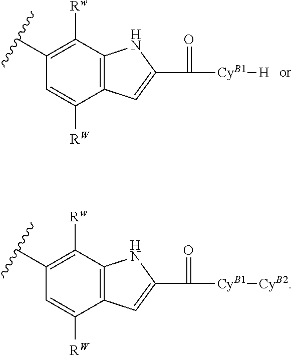

In some embodiments, Ring W and its R^{w} substituents is

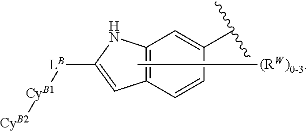

In some embodiments, Ring W and its R^{w} substituents is

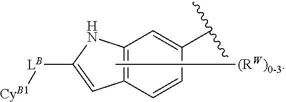

In some embodiments, Ring W and its R^{w} substituents is

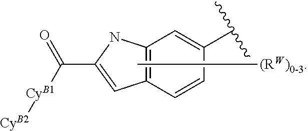

In some embodiments, Ring W and its R^{w} substituents is

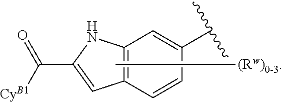

In some embodiments, Ring W and its R^{w} substituents is

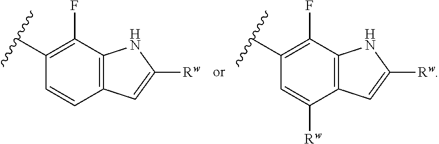

In some embodiments, Ring W and its R^{w} substituents is

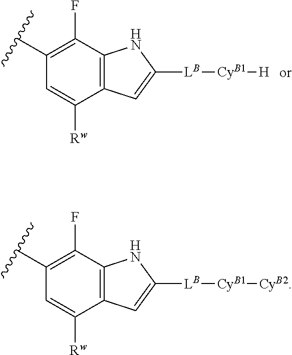

In some embodiments, Ring W and its R^{w} substituents is

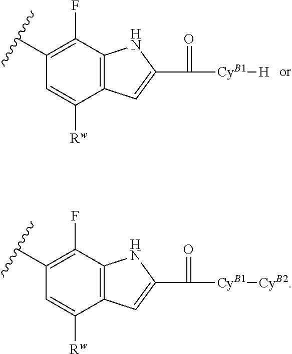

In some embodiments, Ring W and its R^{w} substituents is

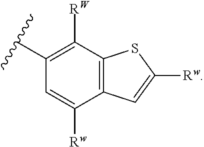

In some embodiments, Ring W and its R^{w} substituents is

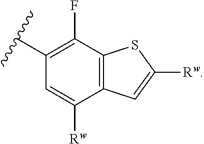

In some embodiments, Ring W and its R^{w} substituents is

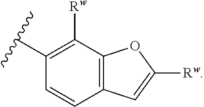

In some embodiments, Ring W and its R^{W} substituents is

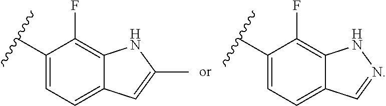 [SMILES: *c1ccc2cc(C)[nH]c2c1F ; "or" ; *c1ccc2cn[nH]c2c1F]

In some embodiment, Ring W is as depicted in the compounds of Table 1B, below.

In some embodiments, Ring W and its R^{w} substituents are

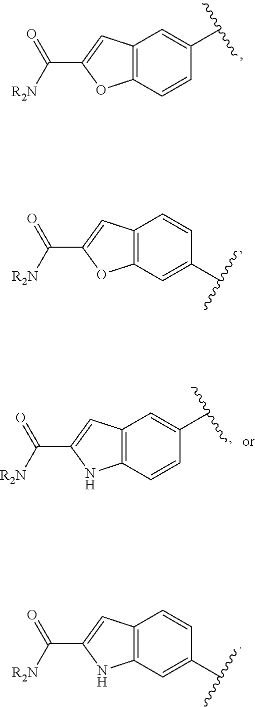

In some embodiments, Ring W and its R^{w} substituents are

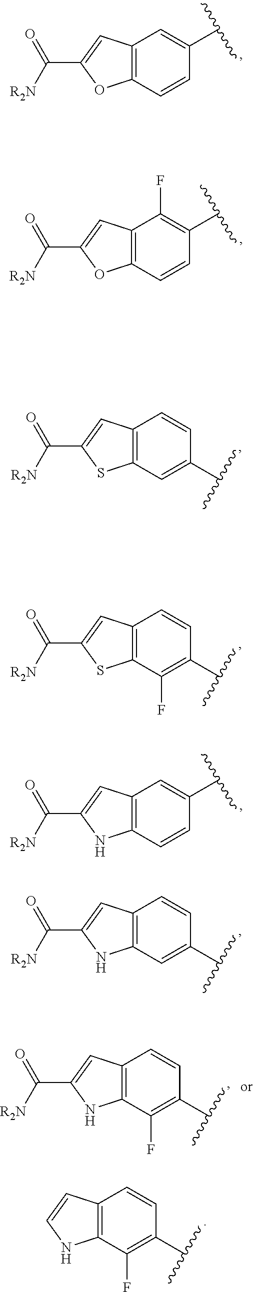

In some embodiments, Ring W and its R^{w} substituents are

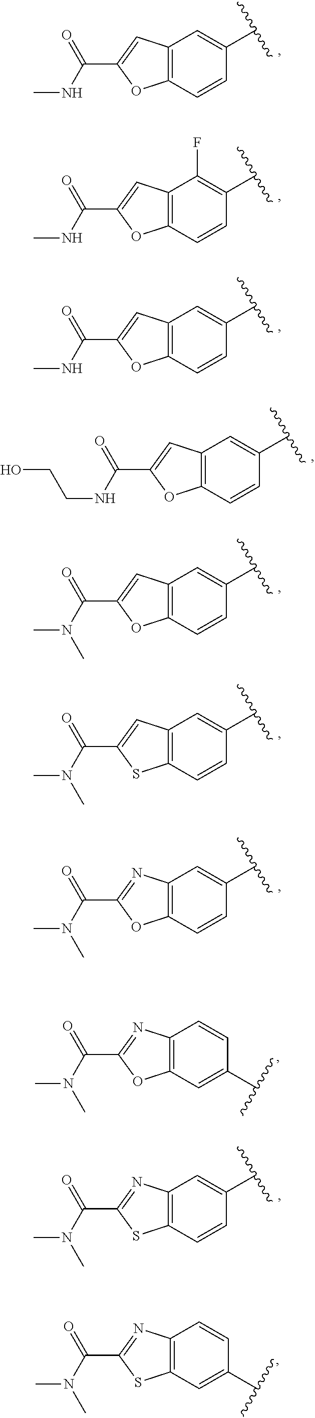

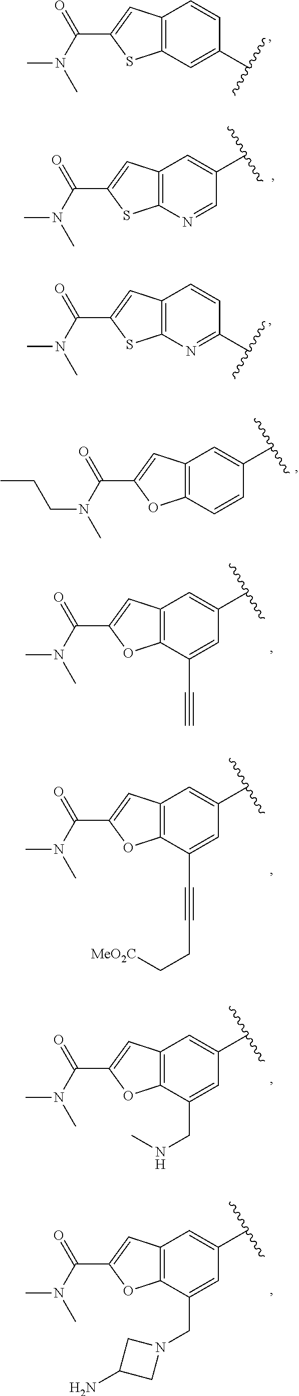

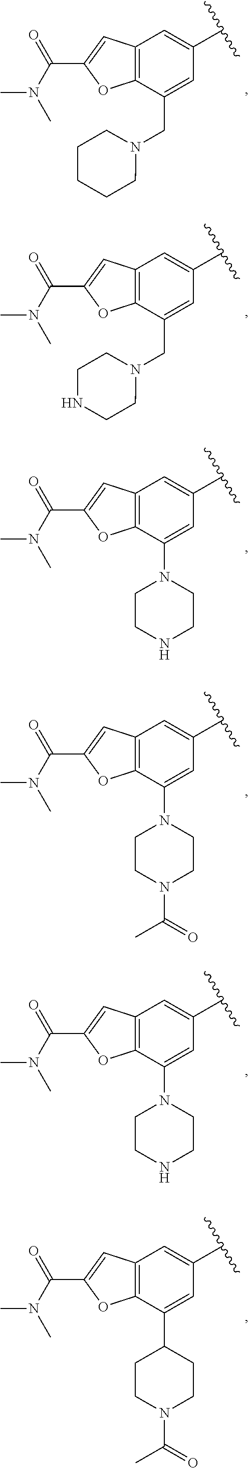 [SMILES: *c1cc(CN2CCCCC2)c2oc(C(=O)N(C)C)cc2c1 ; *c1cc(CN2CCNCC2)c2oc(C(=O)N(C)C)cc2c1 ; *c1cc(N2CCNCC2)c2oc(C(=O)N(C)C)cc2c1 ; *c1cc(N2CCN(C(C)=O)CC2)c2oc(C(=O)N(C)C)cc2c1 ; *c1cc(N2CCNCC2)c2oc(C(=O)N(C)C)cc2c1 ; *c1cc(C2CCN(C(C)=O)CC2)c2oc(C(=O)N(C)C)cc2c1]

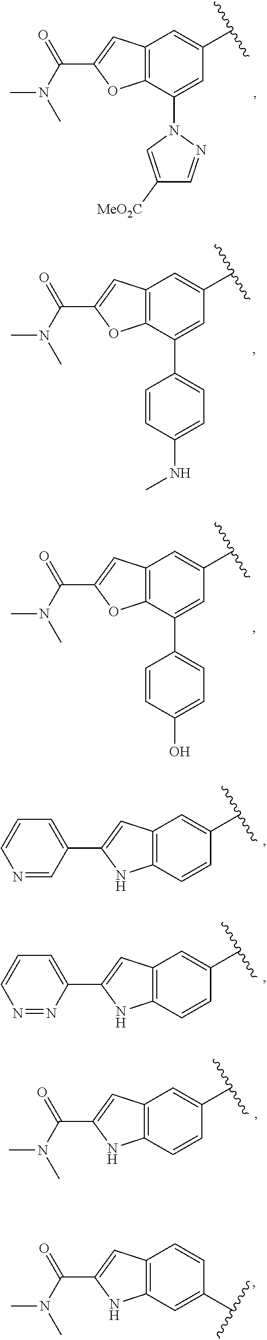

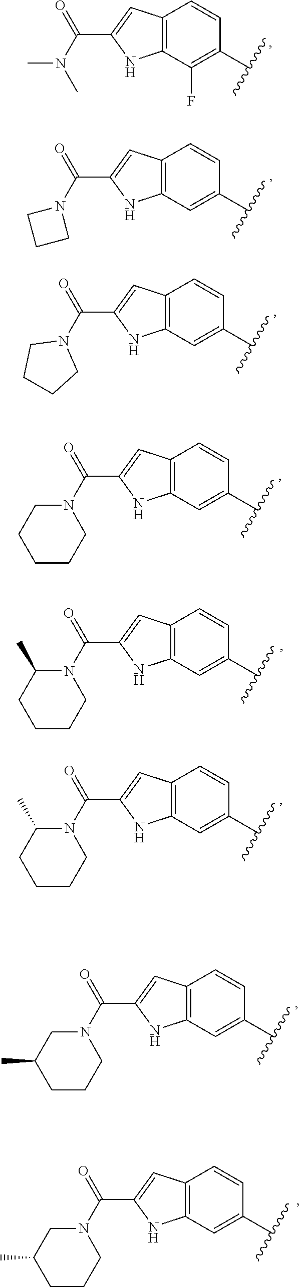

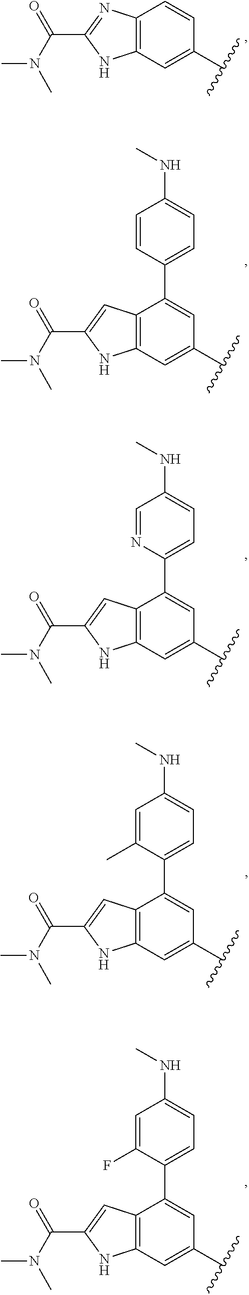 [SMILES: *c1ccc2nc(C(=O)N(C)C)[nH]c2c1 ; *c1cc(-c2ccc(NC)cc2)c2cc(C(=O)N(C)C)[nH]c2c1 ; *c1cc(-c2ccc(NC)cn2)c2cc(C(=O)N(C)C)[nH]c2c1 ; *c1cc(-c2ccc(NC)cc2C)c2cc(C(=O)N(C)C)[nH]c2c1 ; *c1cc(-c2ccc(NC)cc2F)c2cc(C(=O)N(C)C)[nH]c2c1]

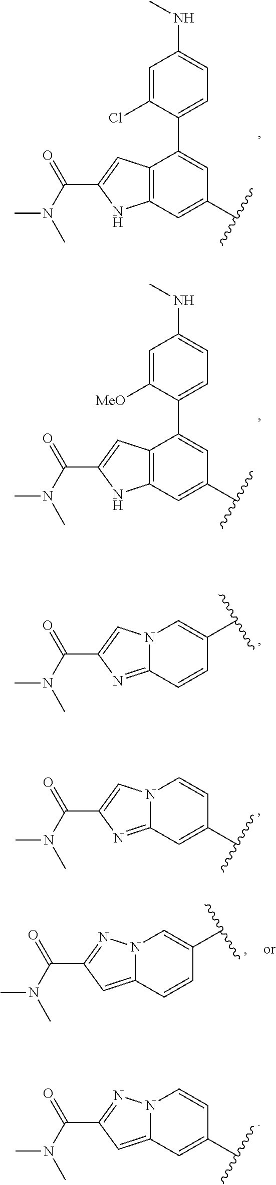 [SMILES: *c1cc(-c2ccc(NC)cc2Cl)c2cc(C(=O)N(C)C)[nH]c2c1 ; *c1cc(-c2ccc(NC)cc2OC)c2cc(C(=O)N(C)C)[nH]c2c1 ; *c1ccc2nc(C(=O)N(C)C)cn2c1 ; *c1ccn2cc(C(=O)N(C)C)nc2c1 ; *c1ccc2cc(C(=O)N(C)C)nn2c1 ; *c1ccn2nc(C(=O)N(C)C)cc2c1]

In some embodiments, Ring W and its R^{w} substituents are

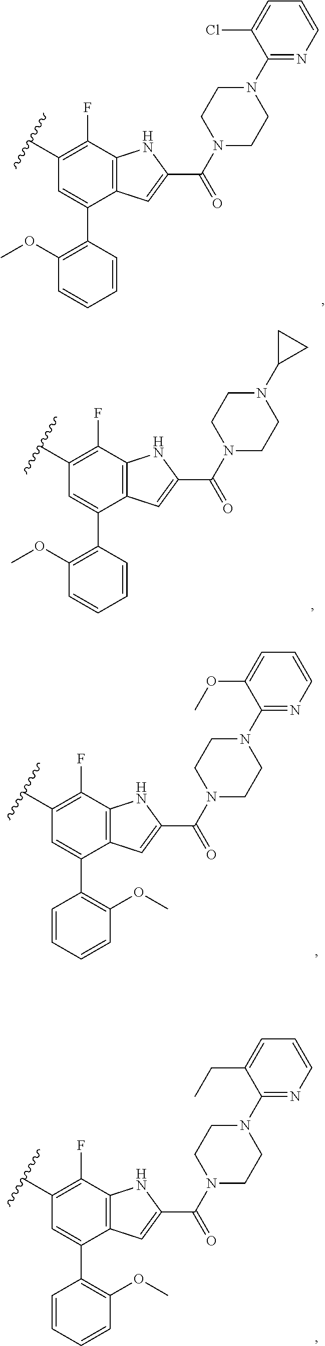 [SMILES: *c1cc(-c2ccccc2OC)c2cc(C(=O)N3CCN(c4ncccc4Cl)CC3)[nH]c2c1F ; *c1cc(-c2ccccc2OC)c2cc(C(=O)N3CCN(C4CC4)CC3)[nH]c2c1F ; *c1cc(-c2ccccc2OC)c2cc(C(=O)N3CCN(c4ncccc4OC)CC3)[nH]c2c1F ; *c1cc(-c2ccccc2OC)c2cc(C(=O)N3CCN(c4ncccc4CC)CC3)[nH]c2c1F]

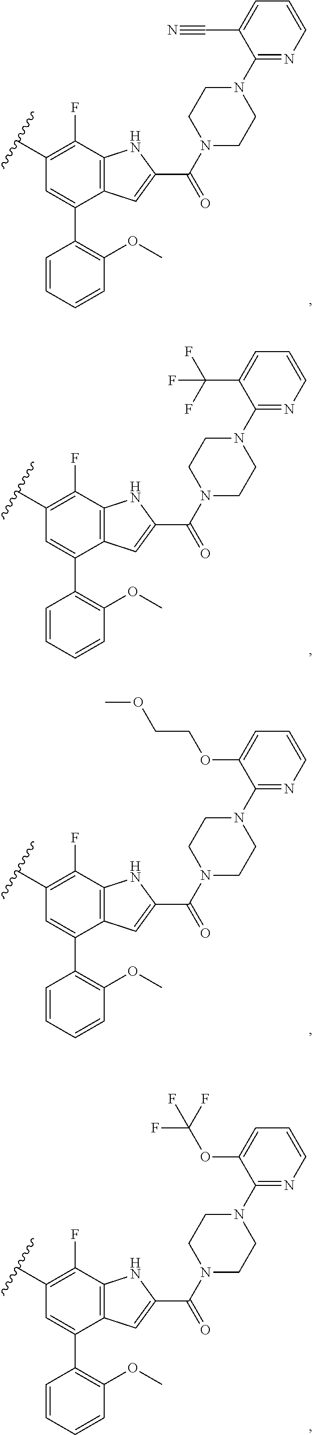 [SMILES: *c1cc(-c2ccccc2OC)c2cc(C(=O)N3CCN(c4ncccc4C#N)CC3)[nH]c2c1F ; *c1cc(-c2ccccc2OC)c2cc(C(=O)N3CCN(c4ncccc4C(F)(F)F)CC3)[nH]c2c1F ; *c1cc(-c2ccccc2OC)c2cc(C(=O)N3CCN(c4ncccc4OCCOC)CC3)[nH]c2c1F ; *c1cc(-c2ccccc2OC)c2cc(C(=O)N3CCN(c4ncccc4OC(F)(F)F)CC3)[nH]c2c1F]

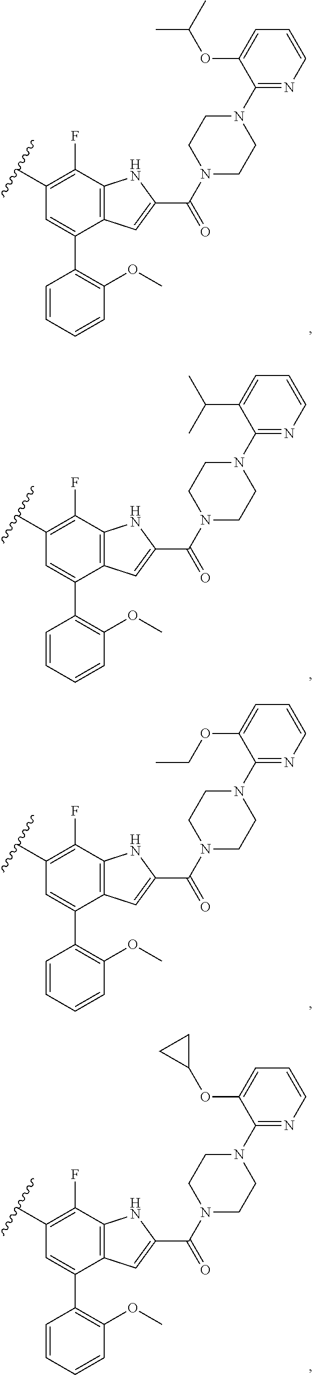 [SMILES: *c1cc(-c2ccccc2OC)c2cc(C(=O)N3CCN(c4ncccc4OC(C)C)CC3)[nH]c2c1F ; *c1cc(-c2ccccc2OC)c2cc(C(=O)N3CCN(c4ncccc4C(C)C)CC3)[nH]c2c1F ; *c1cc(-c2ccccc2OC)c2cc(C(=O)N3CCN(c4ncccc4OCC)CC3)[nH]c2c1F ; *c1cc(-c2ccccc2OC)c2cc(C(=O)N3CCN(c4ncccc4OC4CC4)CC3)[nH]c2c1F]

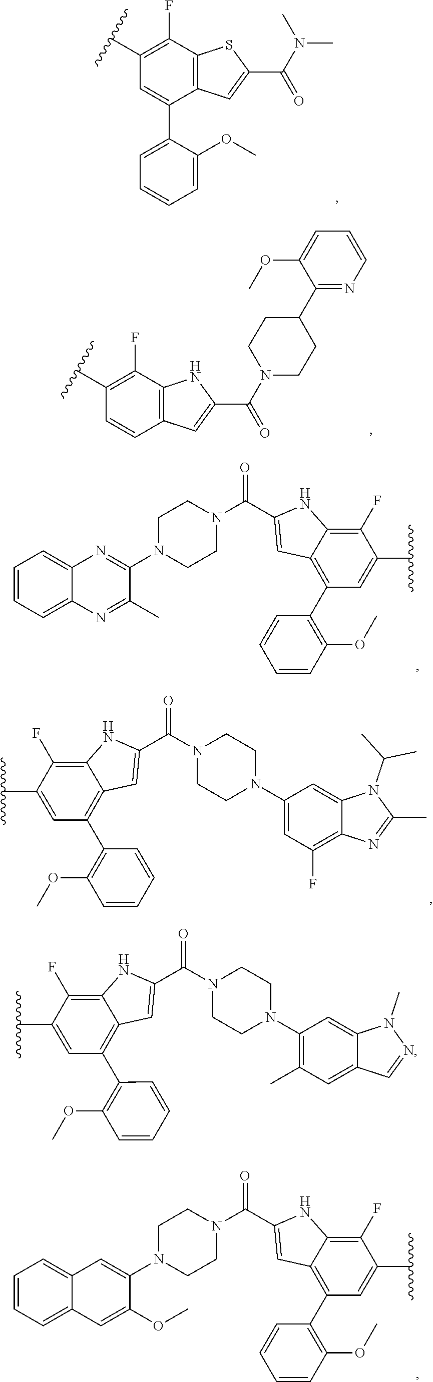

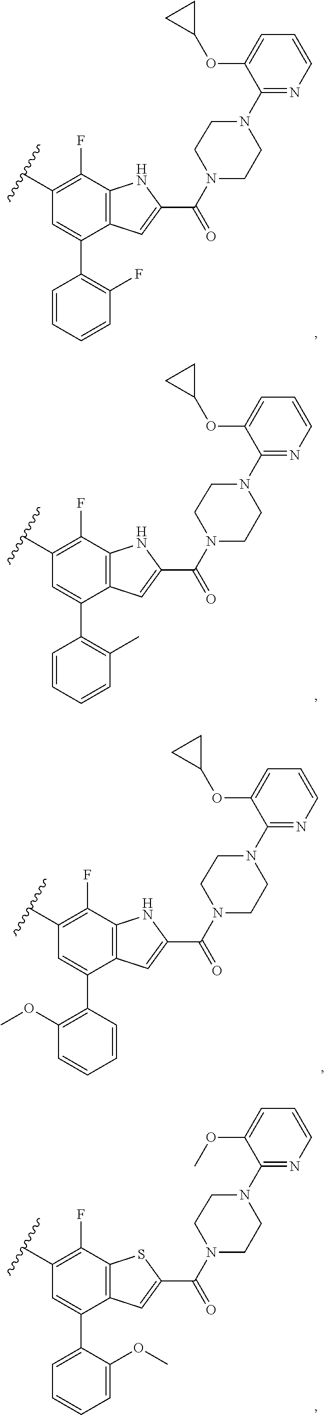 [SMILES: *c1cc(-c2ccccc2F)c2cc(C(=O)N3CCN(c4ncccc4OC4CC4)CC3)[nH]c2c1F ; *c1cc(-c2ccccc2C)c2cc(C(=O)N3CCN(c4ncccc4OC4CC4)CC3)[nH]c2c1F ; *c1cc(-c2ccccc2OC)c2cc(C(=O)N3CCN(c4ncccc4OC4CC4)CC3)[nH]c2c1F ; *c1cc(-c2ccccc2OC)c2cc(C(=O)N3CCN(c4ncccc4OC)CC3)sc2c1F]

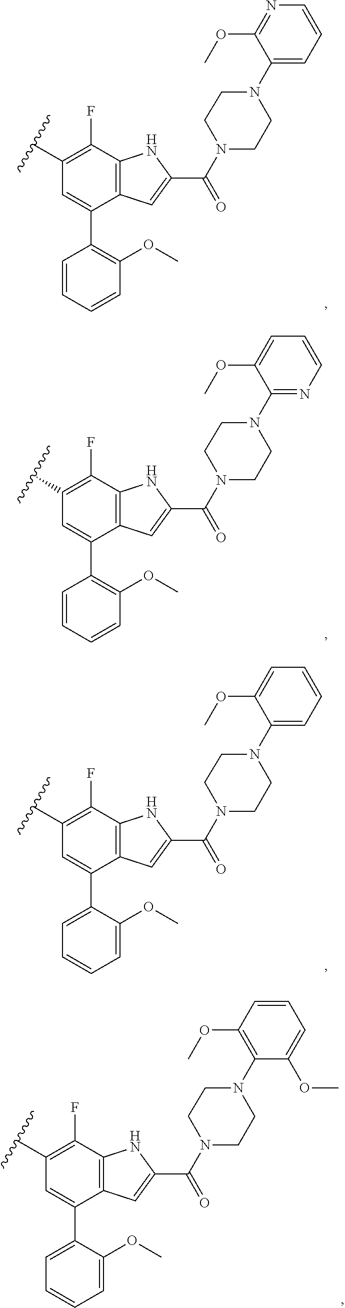

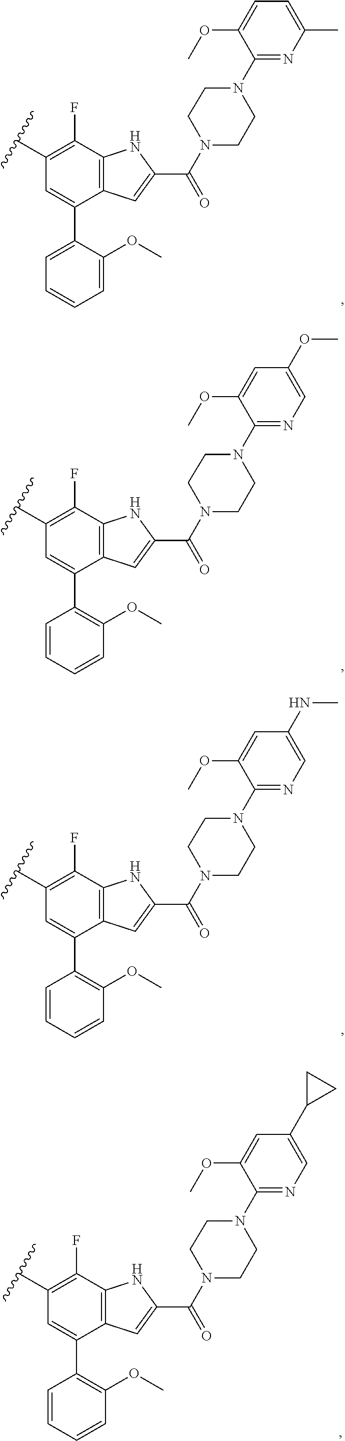 [SMILES: *c1cc(-c2ccccc2OC)c2cc(C(=O)N3CCN(c4nc(C)ccc4OC)CC3)[nH]c2c1F ; *c1cc(-c2ccccc2OC)c2cc(C(=O)N3CCN(c4ncc(OC)cc4OC)CC3)[nH]c2c1F ; *c1cc(-c2ccccc2OC)c2cc(C(=O)N3CCN(c4ncc(NC)cc4OC)CC3)[nH]c2c1F ; *c1cc(-c2ccccc2OC)c2cc(C(=O)N3CCN(c4ncc(C5CC5)cc4OC)CC3)[nH]c2c1F]

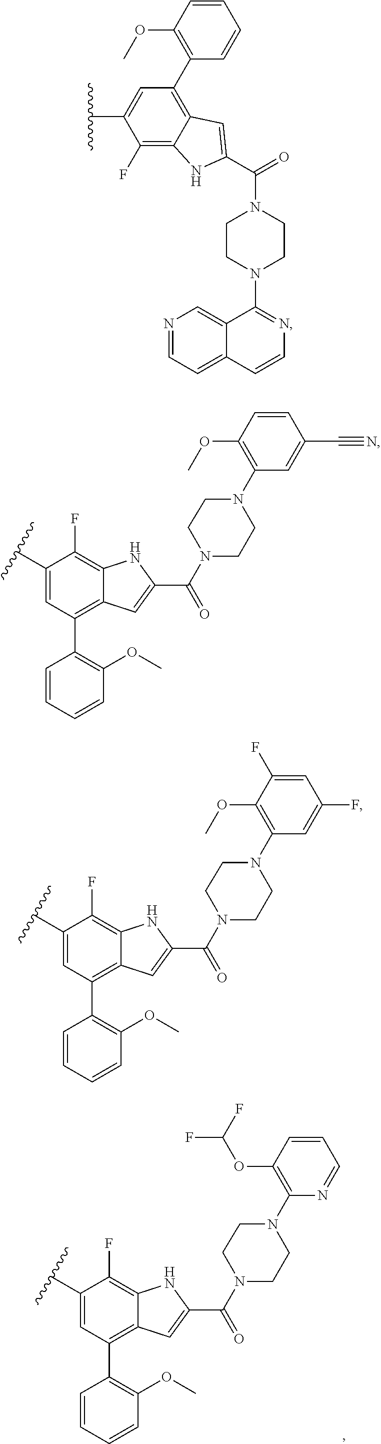

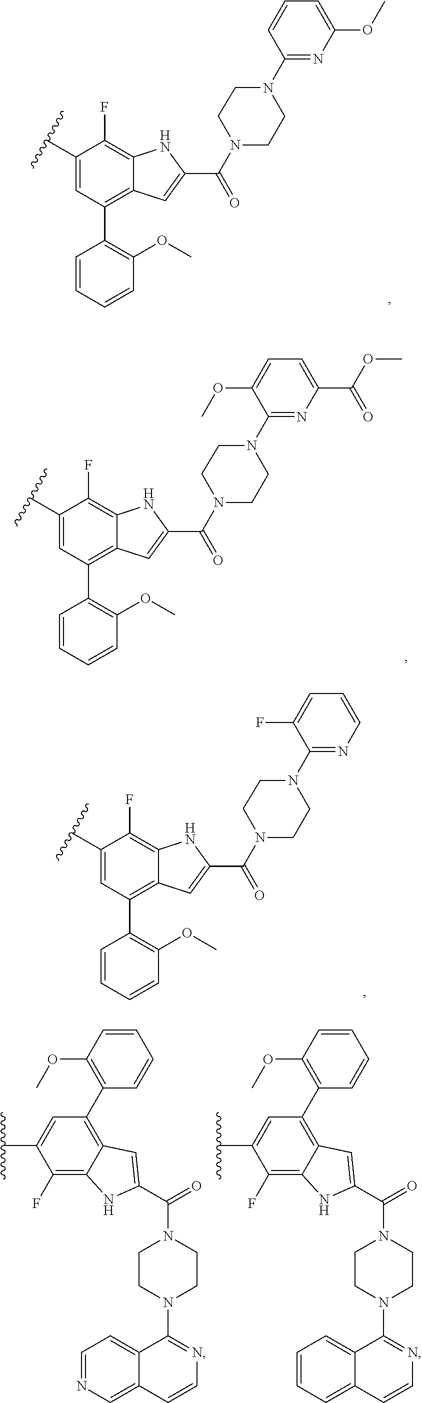

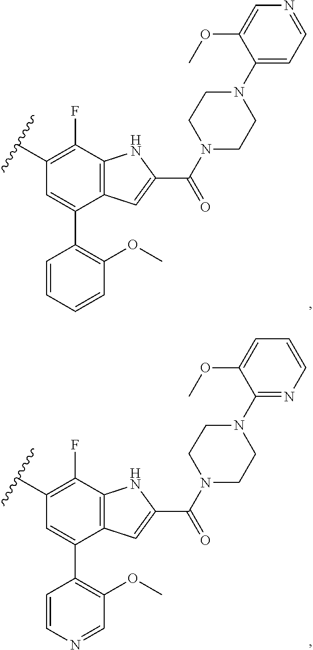 [SMILES: *c1cc(-c2ccccc2OC)c2cc(C(=O)N3CCN(c4ccncc4OC)CC3)[nH]c2c1F ; *c1cc(-c2ccncc2OC)c2cc(C(=O)N3CCN(c4ncccc4OC)CC3)[nH]c2c1F]

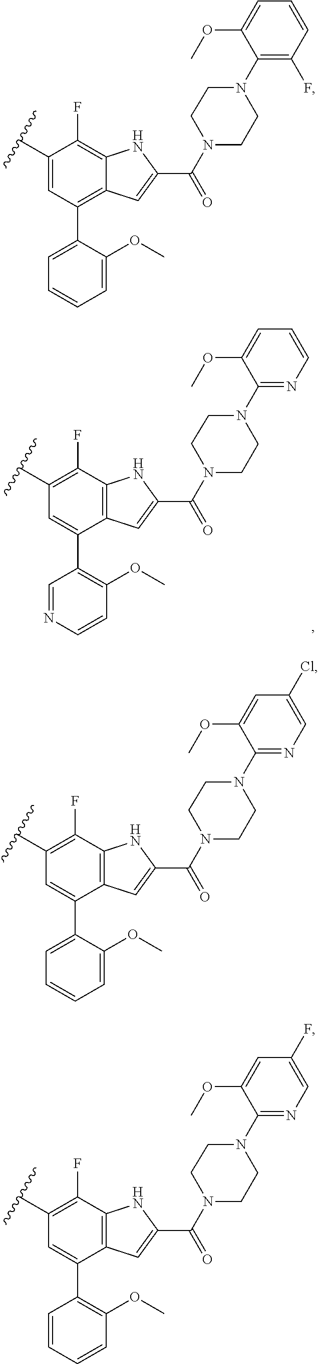 [SMILES: *c1cc(-c2ccccc2OC)c2cc(C(=O)N3CCN(c4c(F)cccc4OC)CC3)[nH]c2c1F ; *c1cc(-c2cnccc2OC)c2cc(C(=O)N3CCN(c4ncccc4OC)CC3)[nH]c2c1F ; *c1cc(-c2ccccc2OC)c2cc(C(=O)N3CCN(c4ncc(Cl)cc4OC)CC3)[nH]c2c1F ; *c1cc(-c2ccccc2OC)c2cc(C(=O)N3CCN(c4ncc(F)cc4OC)CC3)[nH]c2c1F]

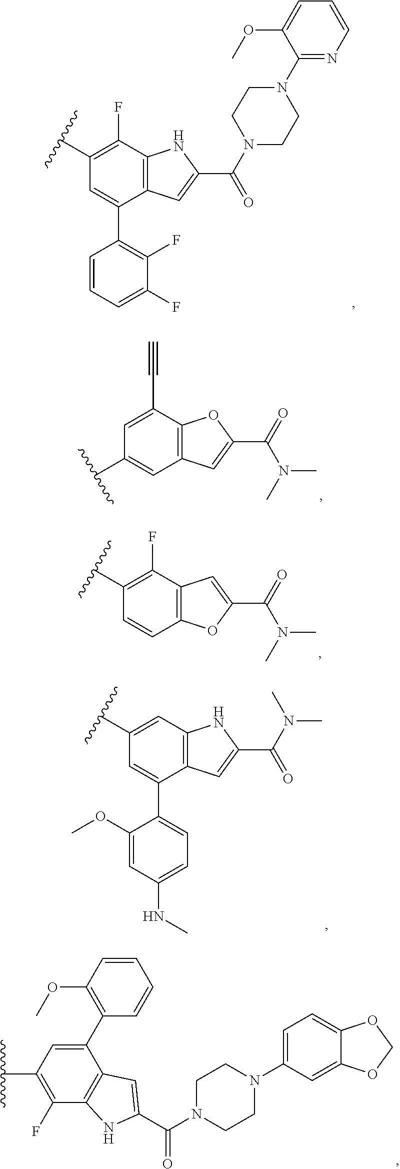

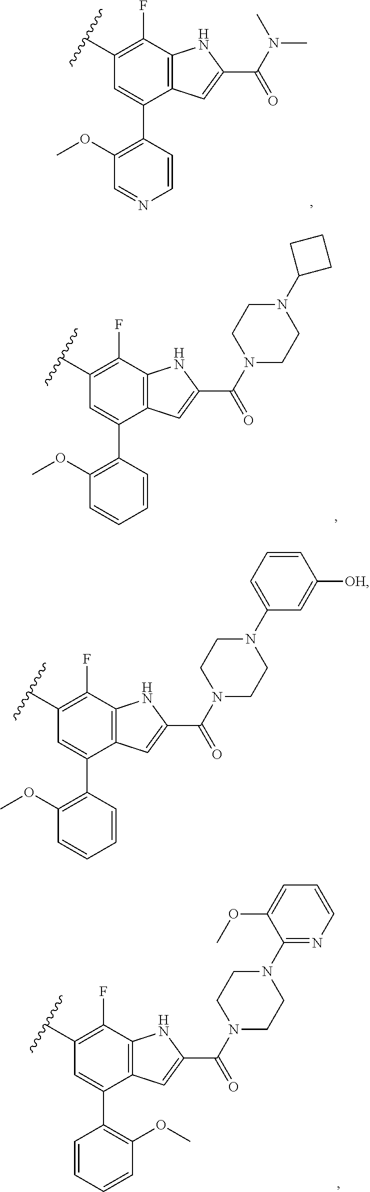

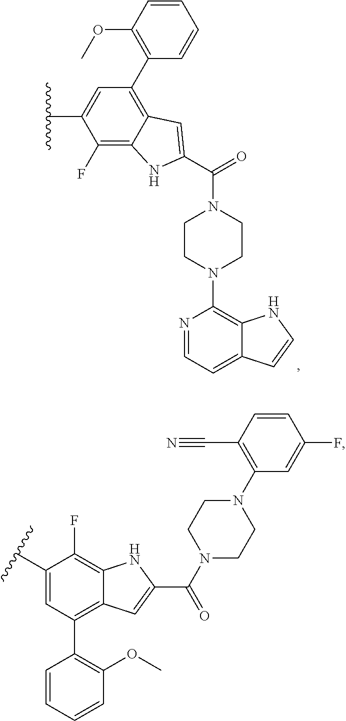 [SMILES: *c1cc(-c2ccccc2OC)c2cc(C(=O)N3CCN(c4nccc5cc[nH]c45)CC3)[nH]c2c1F ; *c1cc(-c2ccccc2OC)c2cc(C(=O)N3CCN(c4cc(F)ccc4C#N)CC3)[nH]c2c1F]

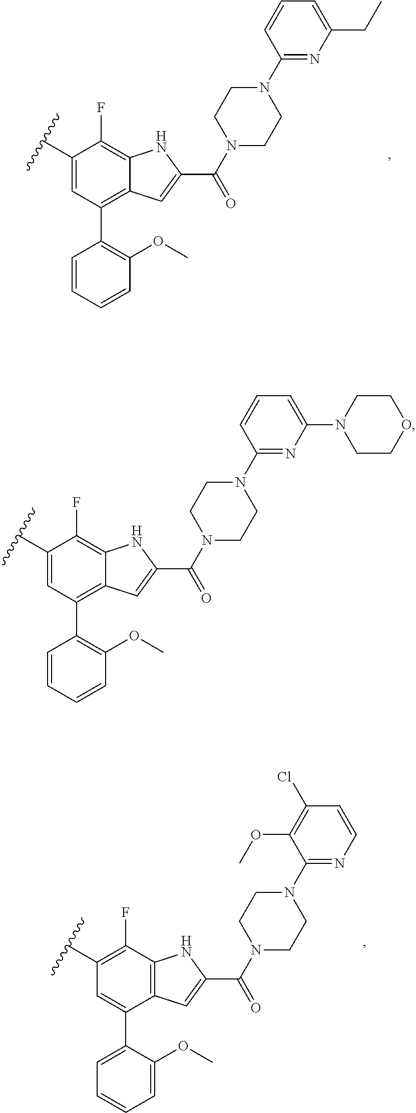 [SMILES: *c1cc(-c2ccccc2OC)c2cc(C(=O)N3CCN(c4cccc(CC)n4)CC3)[nH]c2c1F ; *c1cc(-c2ccccc2OC)c2cc(C(=O)N3CCN(c4cccc(N5CCOCC5)n4)CC3)[nH]c2c1F ; *c1cc(-c2ccccc2OC)c2cc(C(=O)N3CCN(c4nccc(Cl)c4OC)CC3)[nH]c2c1F]

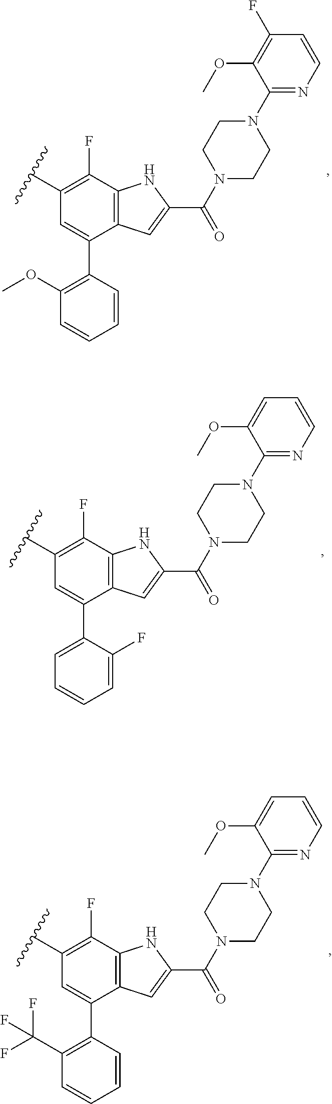 [SMILES: *c1cc(-c2ccccc2OC)c2cc(C(=O)N3CCN(c4nccc(F)c4OC)CC3)[nH]c2c1F ; *c1cc(-c2ccccc2F)c2cc(C(=O)N3CCN(c4ncccc4OC)CC3)[nH]c2c1F ; *c1cc(-c2ccccc2C(F)(F)F)c2cc(C(=O)N3CCN(c4ncccc4OC)CC3)[nH]c2c1F]

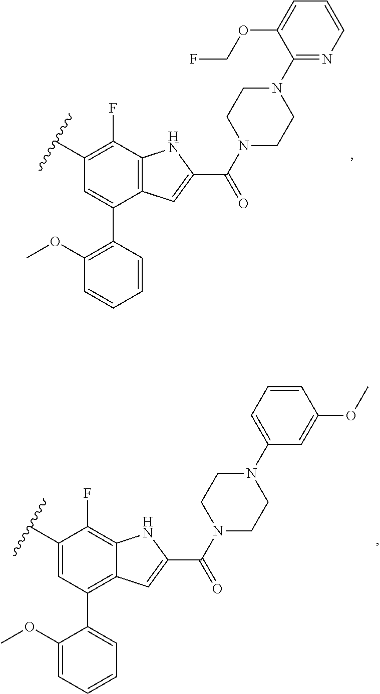

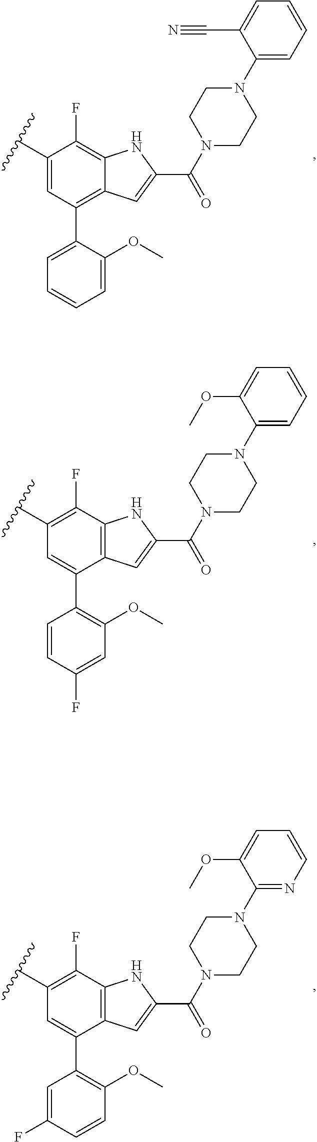 [SMILES: *c1cc(-c2ccccc2OC)c2cc(C(=O)N3CCN(c4ccccc4C#N)CC3)[nH]c2c1F ; *c1cc(-c2ccc(F)cc2OC)c2cc(C(=O)N3CCN(c4ccccc4OC)CC3)[nH]c2c1F ; *c1cc(-c2cc(F)ccc2OC)c2cc(C(=O)N3CCN(c4ncccc4OC)CC3)[nH]c2c1F]

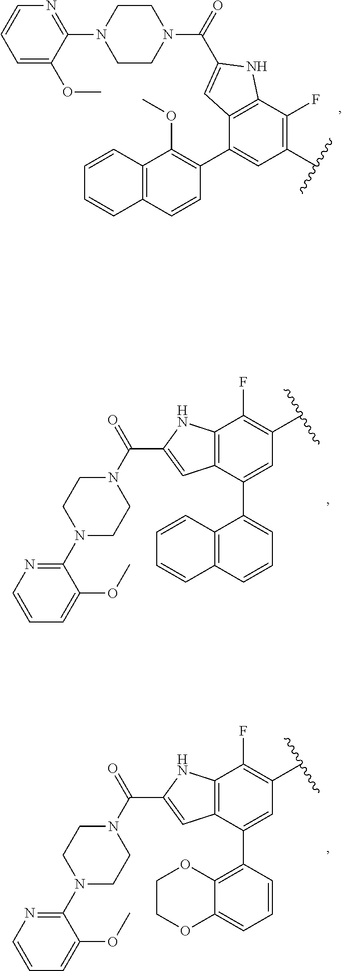 [SMILES: *c1cc(-c2ccc3ccccc3c2OC)c2cc(C(=O)N3CCN(c4ncccc4OC)CC3)[nH]c2c1F ; *c1cc(-c2cccc3ccccc23)c2cc(C(=O)N3CCN(c4ncccc4OC)CC3)[nH]c2c1F ; *c1cc(-c2cccc3c2OCCO3)c2cc(C(=O)N3CCN(c4ncccc4OC)CC3)[nH]c2c1F]

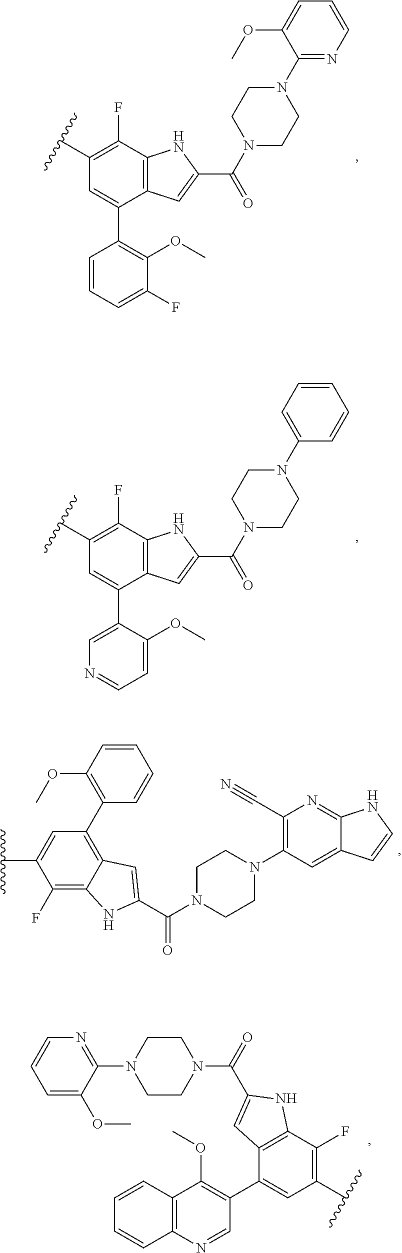 [SMILES: *c1cc(-c2cccc(F)c2OC)c2cc(C(=O)N3CCN(c4ncccc4OC)CC3)[nH]c2c1F ; *c1cc(-c2cnccc2OC)c2cc(C(=O)N3CCN(c4ccccc4)CC3)[nH]c2c1F ; *c1cc(-c2ccccc2OC)c2cc(C(=O)N3CCN(c4cc5cc[nH]c5nc4C#N)CC3)[nH]c2c1F ; *c1cc(-c2cnc3ccccc3c2OC)c2cc(C(=O)N3CCN(c4ncccc4OC)CC3)[nH]c2c1F]

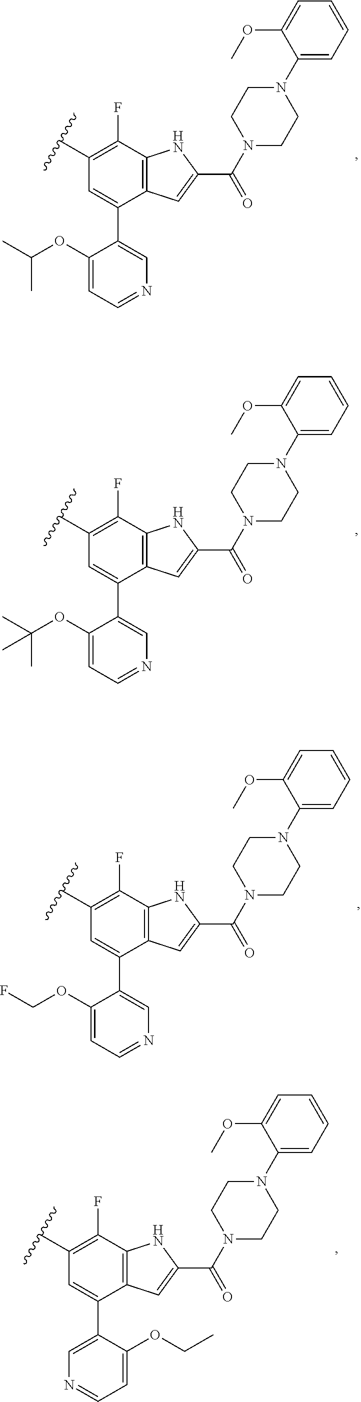 [SMILES: *c1cc(-c2cnccc2OC(C)C)c2cc(C(=O)N3CCN(c4ccccc4OC)CC3)[nH]c2c1F ; *c1cc(-c2cnccc2OC(C)(C)C)c2cc(C(=O)N3CCN(c4ccccc4OC)CC3)[nH]c2c1F ; *c1cc(-c2cnccc2OCF)c2cc(C(=O)N3CCN(c4ccccc4OC)CC3)[nH]c2c1F ; *c1cc(-c2cnccc2OCC)c2cc(C(=O)N3CCN(c4ccccc4OC)CC3)[nH]c2c1F]

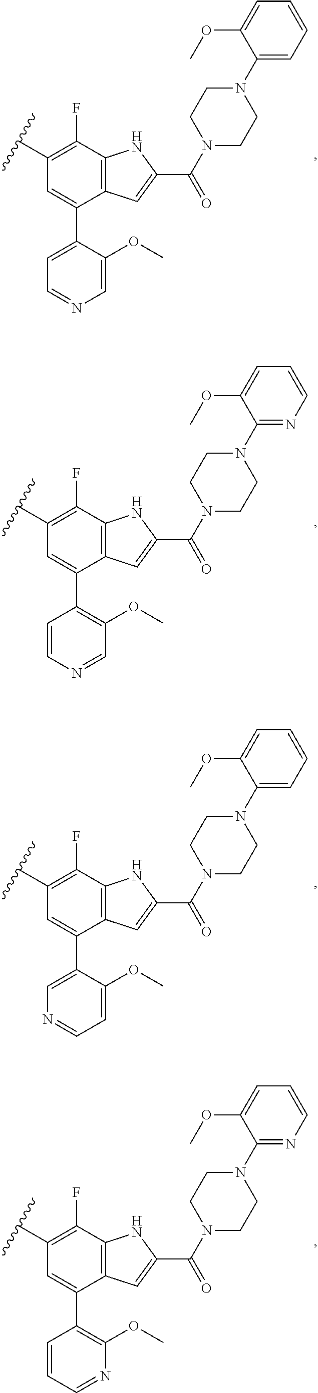 [SMILES: *c1cc(-c2ccncc2OC)c2cc(C(=O)N3CCN(c4ccccc4OC)CC3)[nH]c2c1F ; *c1cc(-c2ccncc2OC)c2cc(C(=O)N3CCN(c4ncccc4OC)CC3)[nH]c2c1F ; *c1cc(-c2cnccc2OC)c2cc(C(=O)N3CCN(c4ccccc4OC)CC3)[nH]c2c1F ; *c1cc(-c2cccnc2OC)c2cc(C(=O)N3CCN(c4ncccc4OC)CC3)[nH]c2c1F]

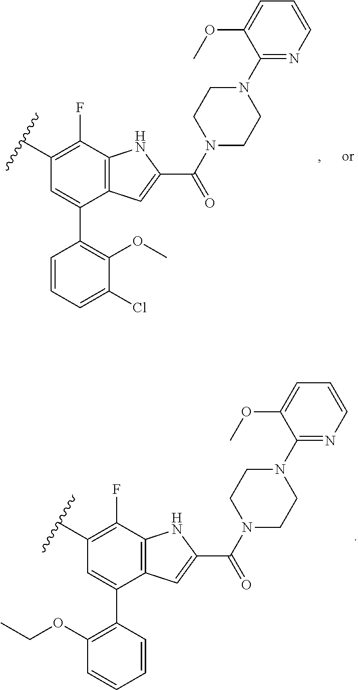 [SMILES: *c1cc(-c2cccc(Cl)c2OC)c2cc(C(=O)N3CCN(c4ncccc4OC)CC3)[nH]c2c1F ; *c1cc(-c2ccccc2OCC)c2cc(C(=O)N3CCN(c4ncccc4OC)CC3)[nH]c2c1F]

In some embodiments, Ring W and its R^{w} substituents are

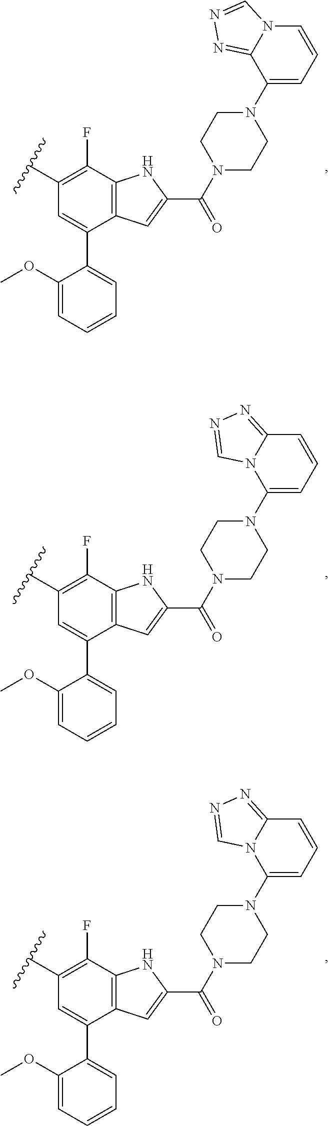 [SMILES: *c1cc(-c2ccccc2OC)c2cc(C(=O)N3CCN(c4cccn5cnnc45)CC3)[nH]c2c1F ; *c1cc(-c2ccccc2OC)c2cc(C(=O)N3CCN(c4cccc5nncn45)CC3)[nH]c2c1F ; *c1cc(-c2ccccc2OC)c2cc(C(=O)N3CCN(c4cccc5nncn45)CC3)[nH]c2c1F]

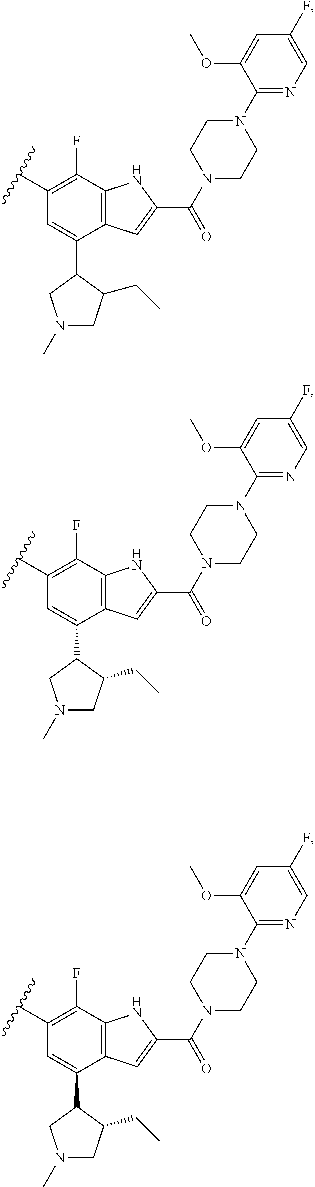

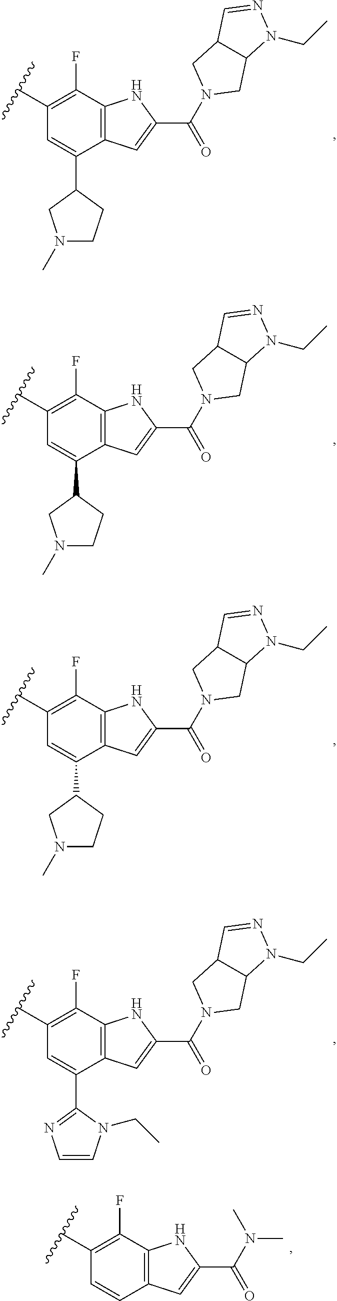

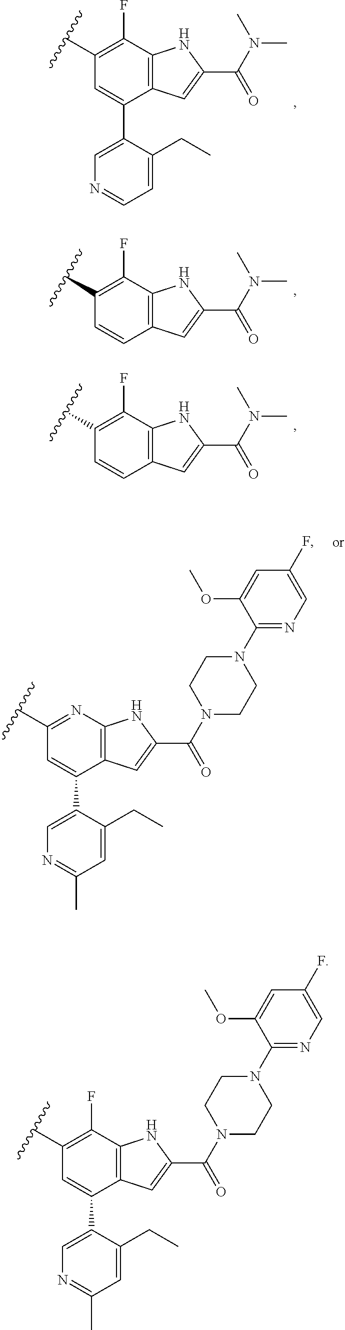

As described above and defined herein, Ring X and its R^{x} substituents is

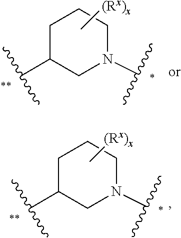

wherein

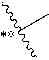

represents a bond to Ring W, and

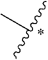

represents a bond to the rest of the molecule.

In some embodiments, Ring X and its R^{x} substituents is

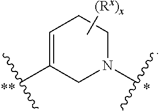

In some embodiments, Ring X and its R^{x} substituents is

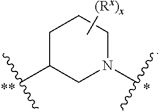

In some embodiments, Ring X and its R^{x} substituents is

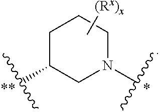

In some embodiments, Ring X and its R^{x} substituents is

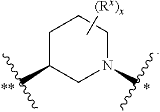

In some embodiment, Ring X is as depicted in the compounds of Table 1B, below.

In some embodiments, Ring X and its R^{x} substituents are

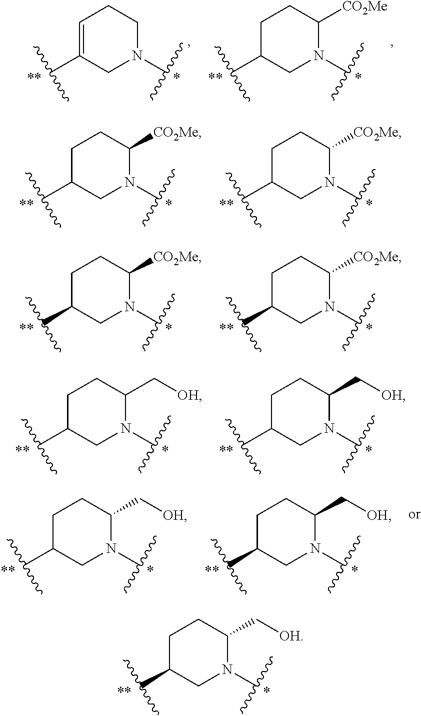

In some embodiments, Ring X and its R^{x} substituents are

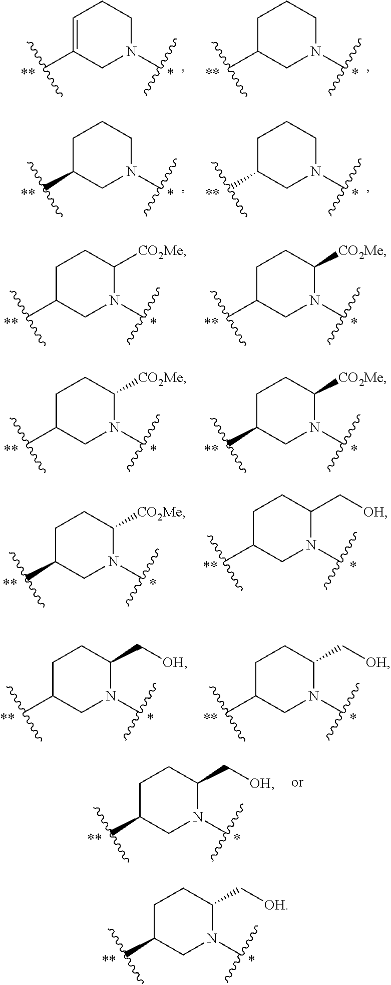

In some embodiments, Ring X and its R^{x} substituents are

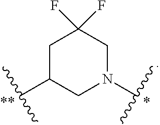 [SMILES: *C1CN(*)CC(F)(F)C1 ; "."]

As described above and defined herein, Ring Y is a ring selected from phenyl, 3-8 membered saturated or partially unsaturated monocyclic carbocyclyl or heterocyclyl with 1-3 heteroatoms independently selected from nitrogen, oxygen, and sulfur, and 5-6 membered monocyclic heteroaryl with 1-4 heteroatoms independently selected from nitrogen, oxygen and sulfur.

In some embodiments, Ring Y is phenyl.

In some embodiments, Ring Y is a 3-8 membered saturated or partially unsaturated monocyclic carbocyclyl. In some embodiments, Ring Y is a 3-6 membered saturated or partially unsaturated monocyclic carbocyclyl. In some embodiments, Ring Y is cyclopropyl. In some embodiments, Ring Y is cyclobutyl. In some embodiments, Ring Y is cyclopentyl. In some embodiments, Ring Y is hexyl.

In some embodiments, Ring Y is a 5-6 membered saturated or partially unsaturated monocyclic carbocyclyl. In some embodiments, Ring Y is a 6-7 membered saturated or partially unsaturated monocyclic carbocyclyl. In some embodiments, Ring Y is a 3 membered saturated or partially unsaturated monocyclic carbocyclyl. In some embodiments, Ring Y is a 4 membered saturated or partially unsaturated monocyclic carbocyclyl. In some embodiments, Ring Y is a 5 membered saturated or partially unsaturated monocyclic carbocyclyl. In some embodiments, Ring Y is a 6 membered saturated or partially unsaturated monocyclic carbocyclyl. In some embodiments, Ring Y is a 7 membered saturated or partially unsaturated monocyclic carbocyclyl. In some embodiments, Ring Y is cyclopropyl, cyclobutyl, cyclopentyl, cyclohexyl, or cycloheptyl.

In some embodiments, Ring Y is a 5-6 membered monocyclic heteroaryl with 1-4 heteroatoms independently selected from nitrogen, oxygen and sulfur.

In some embodiments, Ring Y is a 5-membered monocyclic heteroaryl ring with 1 heteroatom selected from nitrogen, oxygen, and sulfur. In some embodiments, Ring Y is a 5-membered monocyclic heteroaryl ring with 2 heteroatoms independently selected from nitrogen, oxygen, and sulfur. In some embodiments, Ring Y is a 5-membered monocyclic heteroaryl ring with 3 heteroatoms independently selected from nitrogen, oxygen, and sulfur. In some embodiments, Ring Y is a 5-membered monocyclic heteroaryl ring with 4 heteroatoms independently selected from nitrogen, oxygen, and sulfur. In some embodiments, Ring Y is a 5-membered monocyclic heteroaryl ring with 2-3 nitrogen heteroatoms.

In some embodiments, Ring Y is a 6-membered monocyclic heteroaryl with 1-4 heteroatoms independently selected from nitrogen, oxygen, and sulfur. In some embodiments, Ring Y is a 6-membered monocyclic heteroaryl with 1-4 nitrogen heteroatoms. In some embodiments, Ring Y is pyridinyl, pyrimidinyl, pyridazinyl, or triazinyl.

In some embodiments, Ring Y is a 3-8 membered saturated or partially unsaturated monocyclic heterocyclyl with 1-3 heteroatoms independently selected from nitrogen, oxygen, and sulfur.

In some embodiments, Ring Y is

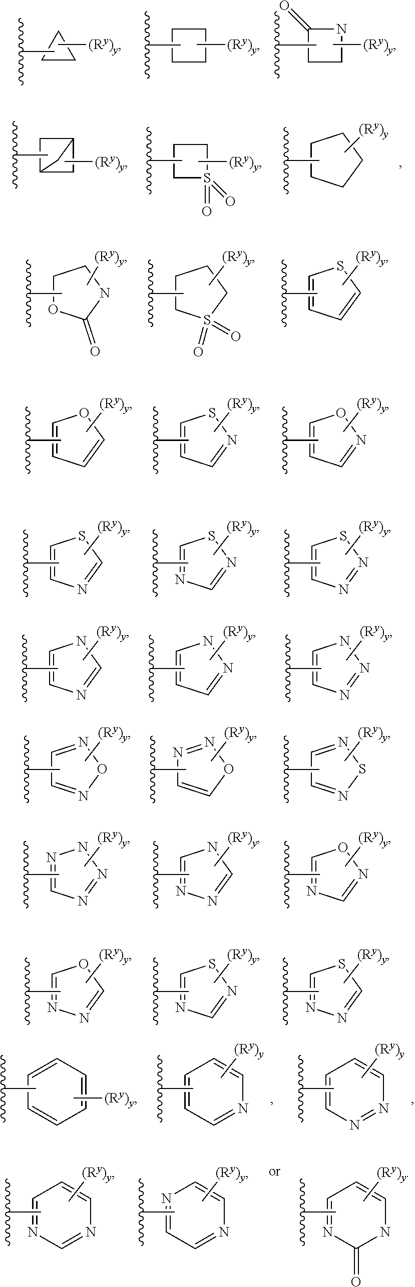

In some embodiments, Ring Y is

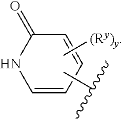

In some embodiments, Ring Y is

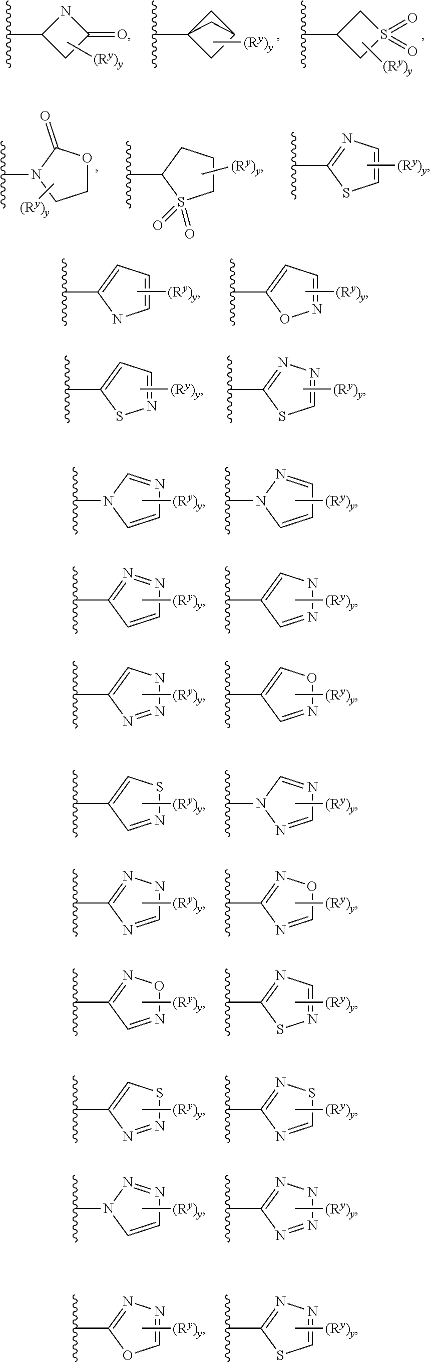

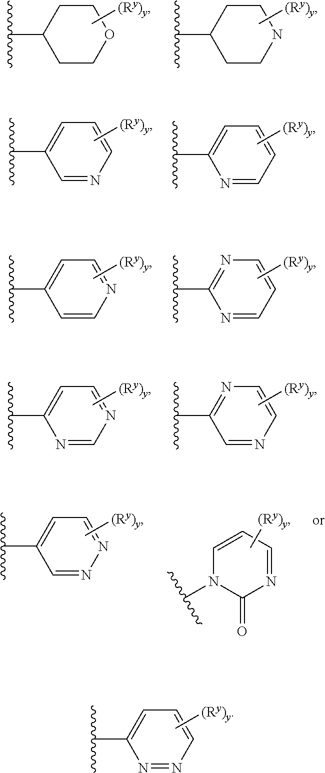

In some embodiment, Ring Y is

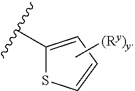

In some embodiment, Ring Y is as depicted in the compounds of Table 1B, below.

In some embodiments, Ring Y and its R^{y} substituents are

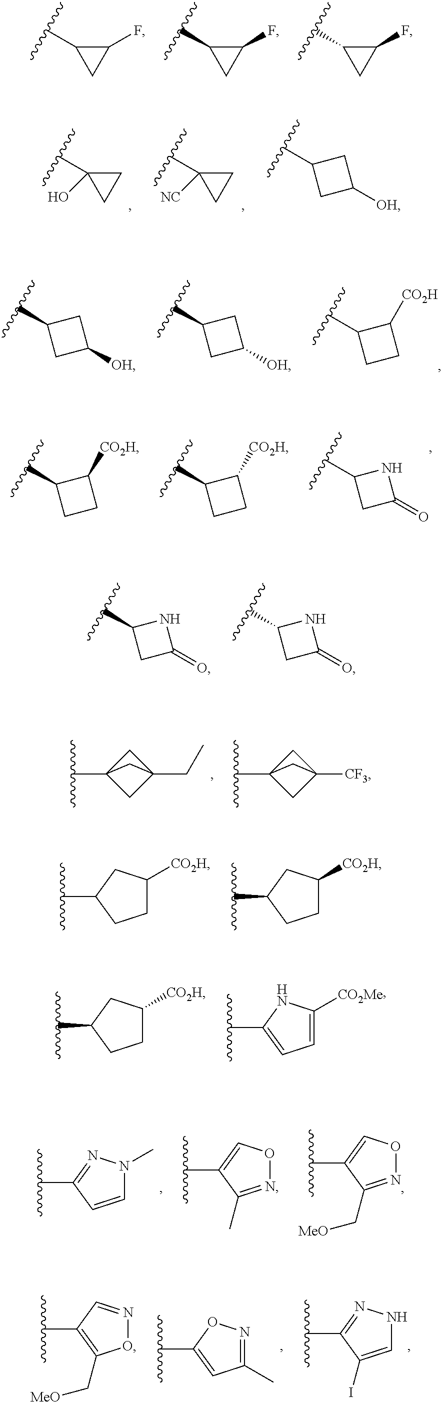

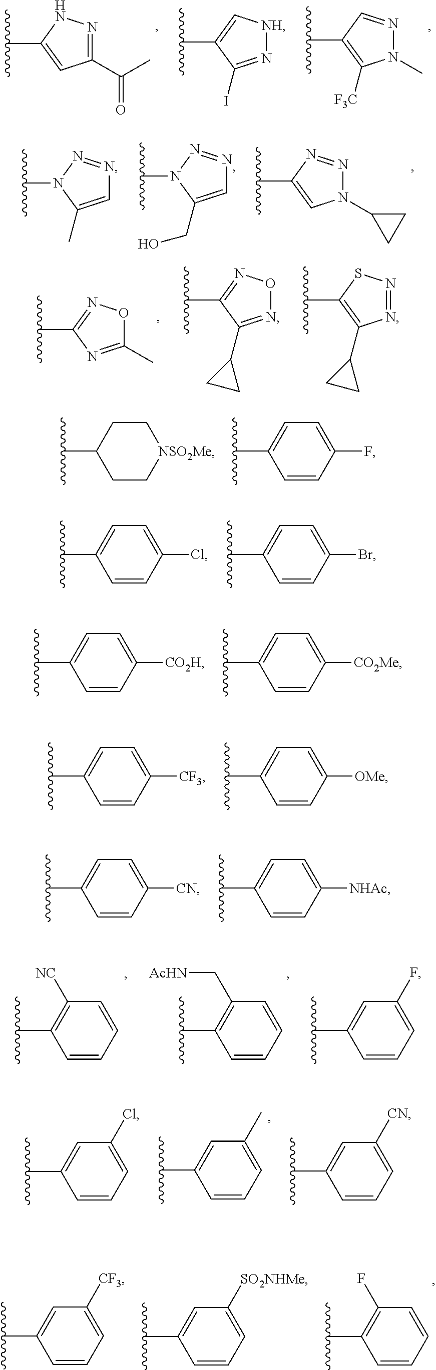

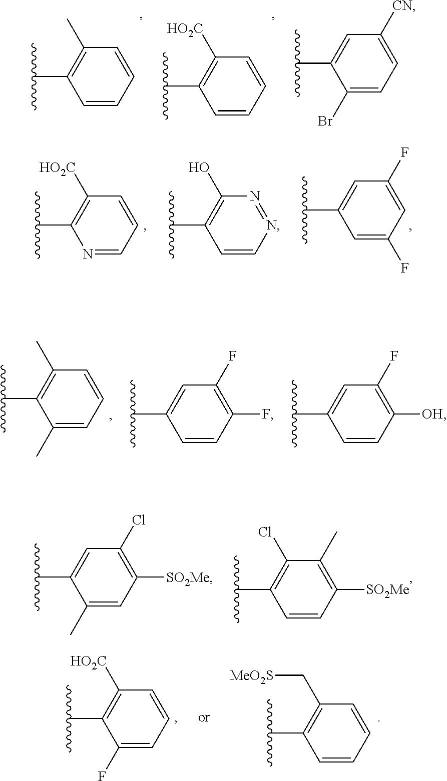

In some embodiments Ring Y and its R^{y} substituents are

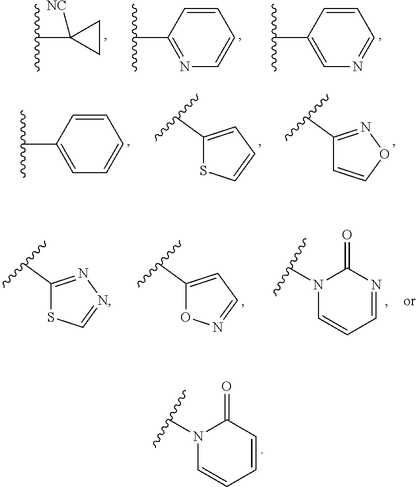

In some embodiments, Ring Y is

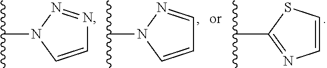

In some embodiments, Ring Y and its R^{y} substituents are

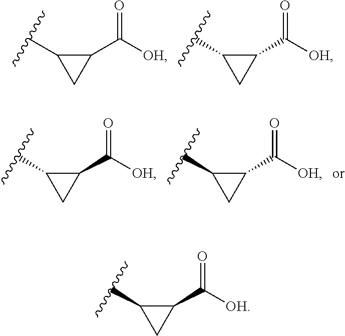 [SMILES: *C1CC1C(=O)O ; *[C@H]1C[C@H]1C(=O)O ; *[C@H]1C[C@@H]1C(=O)O ; *[C@@H]1C[C@H]1C(=O)O ; *[C@@H]1C[C@@H]1C(=O)O]

As described above and defined herein, R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, R^{x} and R^{y} are independently selected from R^{A}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

As described above and defined herein, each R^{w} is independently selected from hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, each R^{w} is independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, each R^{w} is independently selected from R^{A}, R^{B}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, one or more of R^{w}, R^{x}, and R^{y} is hydrogen. In some embodiments, one or more of R^{w}, R^{x}, and R^{y} is R^{A}. In some embodiments, one or more of R^{w}, R^{x}, and R^{y} is halogen. In some embodiments, one or more of R^{w}, R^{x}, and R^{y} is —CN. In some embodiments, one or more of R^{w}, R^{x}, and R^{y} is —NO₂. In some embodiments, one or more of R^{w}, R^{x}, and R^{y} is —OR. In some embodiments, one or more of R^{w}, R^{x}, and R^{y} is —SR. In some embodiments, one or more of R^{w}, R^{x}, and R^{y} is —NR₂. In some embodiments, one or more of R^{w}, R^{x}, and R^{y} is —SiR₃. In some embodiments, one or more of R^{w}, R^{x}, and R^{y} is —S(O)₂R. In some embodiments, one or more of R^{w}, R^{x}, and R^{y} is —S(O)₂NR₂. In some embodiments, one or more of R^{w}, R^{x}, and R^{y} is —S(O)(NR)R. In some embodiments, one or more of R^{w}, R^{x}, and R^{y} is —S(O)R. In some embodiments, one or more of R^{w}, R^{x}, and R^{y} is —C(O)R. In some embodiments, one or more of R^{w}, R^{x}, and R^{y} is —C(O)OR. In some embodiments, one or more of R^{w}, R^{x}, and R^{y} is —C(O)NR₂. In some embodiments, one or more of R^{w}, R^{x}, and R^{y} is —C(O)NROR. In some embodiments, one or more of R^{w}, R^{x}, and R^{y} is —OC(O)R. In some embodiments, one or more of R^{w}, R^{x}, and R^{y} is —OC(O)NR₂. In some embodiments, one or more of R^{w}, R^{x}, and R^{y} is —OP(O)R₂. In some embodiments, one or more of R^{w}, R^{x}, and R^{y} is —OP(O)(OR)₂. In some embodiments, one or more of R^{w}, R^{x}, and R^{y} is —NRC(O)OR. In some embodiments, one or more of R^{w}, R^{x}, and R^{y} is —NRC(O)R. In some embodiments, one or more of R^{w}, R^{x}, and R^{y} is —NRC(O)N(R)₂. In some embodiments, one or more of R^{w}, R^{x}, and R^{y} is —NRS(O)₂R.

In some embodiments, R^{w}—C(O)OR. In some embodiments, R^{w} is —C(O)NR₂. In some embodiments, R^{w} is an optionally substituted phenyl. In some embodiments, R^{w} is an optionally substituted 5-6 membered heteroaryl ring having 1-4 heteroatoms independently selected from nitrogen, oxygen, and sulfur.

In some embodiments, R^{w} is R^{B}. In some embodiments, R^{w} is R^{A}.

In some embodiments, R^{w} is fluoro, chloro, or bromo.

In some embodiments, R^{w} is —C(O)NHR. In some embodiments, R^{w} is —C(O)NHR, wherein R of R^{w} is optionally substituted C₁₋₆ aliphatic. In some embodiments, R^{w} is —C(O)NHR, wherein R of R^{w} is C₁₋₆ aliphatic, optionally substituted with —CN.

In some embodiments, R^{w} is optionally substituted C₁₋₆ aliphatic. In some embodiments, R^{w} is C₁₋₆ aliphatic, optionally substituted with —C(O)N(R^{◯})₂. In some embodiments, R^{w} is C₁₋₆ aliphatic, optionally substituted with —NR^{◯}(O)N(R^{◯})₂. In some embodiments, R^{w} is

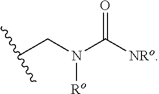

In some such embodiments, R^{◯} is hydrogen or C₁₋₆ aliphatic. In some embodiments, R^{w} is C₁₋₆ aliphatic optionally substituted with —OR^{◯}, wherein R^{◯} is hydrogen or C₁₋₆ aliphatic. In some embodiments, R^{w} is C₁₋₆ aliphatic optionally substituted with halogen (e.g., fluoro). In some embodiments, R^{w} is —CH₂F, —CHF₂, or —CF₃.

In some embodiments, R^{w} is C₁₋₆ aliphatic optionally substituted with halogen (e.g., fluoro). In some embodiments, R^{w} is

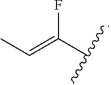 [SMILES: */C(F)=C/C ; "."]

In some embodiments, R^{w} is C₁₋₆ aliphatic optionally substituted with halogen (e.g., fluoro) or —NR^{◯}₂. In some embodiments, R^{w} is C₁₋₆ aliphatic optionally substituted with halogen (e.g., fluoro) and —NR^{◯}₂. In some embodiments, R^{w} is C₁₋₆ aliphatic optionally substituted with halogen (e.g., fluoro) or —NR^{◯}₂, wherein each R^{◯} is independently hydrogen or C₁₋₆ aliphatic. In some embodiments, R^{w} is C₁₋₆ aliphatic optionally substituted with halogen (e.g., fluoro) and —NR^{◯}₂, wherein each R^{◯} is independently hydrogen or C₁₋₆ aliphatic. In some embodiments, R^{w} is

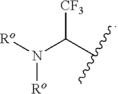

In some embodiments, R^{w} is an optionally substituted phenyl.

In some embodiments, R^{w} is an optionally substituted 3-7 membered saturated or partially unsaturated carbocyclic. In some embodiments, R^{w} is an optionally substituted 3-7 membered saturated or partially unsaturated carbocyclic. In some embodiments, R^{w} is an optionally substituted cyclopropyl. In some embodiments, R^{w} is an optionally substituted cyclobutyl. In some embodiments, R^{w} is an optionally substituted cyclopentyl. In some embodiments, R^{w} is an optionally substituted cyclohexyl. In some embodiments, R^{w} is an optionally substituted cyclopropenyl. In some embodiments, R^{w} is an optionally substituted cyclobutenyl. In some embodiments, R^{w} is an optionally substituted cyclopentenyl. In some embodiments, R^{w} is an optionally substituted cyclohexenyl.

In some embodiments, R^{w} is an optionally substituted 3-7 membered saturated or partially unsaturated heterocyclic ring having 1-2 heteroatoms independently selected from nitrogen, oxygen, and sulfur. In some embodiments, R^{w} is an optionally substituted 5-6 membered saturated or partially unsaturated heterocyclic ring having 1-2 heteroatoms independently selected from nitrogen, oxygen, and sulfur.

In some embodiments, R^{w} is an optionally substituted 4 membered saturated or partially unsaturated heterocyclic ring having 1-2 heteroatoms independently selected from nitrogen, oxygen, and sulfur. In some embodiments, R^{w} is an optionally substituted azetidinyl, oxetanyl, or thietanyl.

In some embodiments, R^{w} is an optionally substituted 5 membered saturated or partially unsaturated heterocyclic ring having 1-2 heteroatoms independently selected from nitrogen, oxygen, and sulfur. In some embodiments, R^{w} is an optionally substituted pyrrolidinyl, pyrrolinyl, pyrazolidinyl, pyrazolinyl, imidazolidinyl, or imidazolinyl. In some embodiments, R^{w} is an optionally substituted dihydropyridinyl, pyrrolidinyl, dihydrofuranyl, tetrahydrofuranyl, dihydrothiophenyl, or tetrahydrothiophenyl.

In some embodiments, R^{w} is an optionally substituted 6 membered saturated or partially unsaturated heterocyclic ring having 1-2 heteroatoms independently selected from nitrogen, oxygen, and sulfur. In some embodiments, R^{w} is an optionally substituted piperidinyl, piperazinyl, tetrahydropyranyl, 2H-pyranyl, 4H-pyranyl, 1,4-dioxanyl, 1,4-dioxinyl, thianyl, 2H-thiopyranyl, 4H-thiopyranyl, 1,3-dithanyl, 1,4-dithanyl, morpholinyl, or thiomorpholinyl. In some embodiments, R^{w} is an optionally substituted dihydropyridinyl, tetrahydropyridinyl, dihydropyranyl, tetrahydropyranyl, dihydrothiopyranyl, or tetrahydrothiopyranyl.

In some embodiments, R^{w} is an optionally substituted

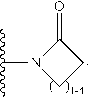

In some embodiments, R^{w} is an optionally substituted

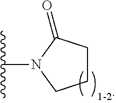

In some embodiments, R^{w} is an optionally substituted

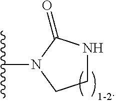

In some embodiments, R^{w} is

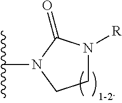

In some embodiments, R^{w} is optionally substituted

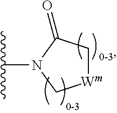

where W^{m} is O, S, C(O), or NR^{◯}, wherein R^{◯} is as described above and defined herein. In some embodiments, R^{w} is

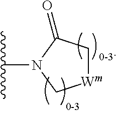

In some embodiments, R^{w} is optionally substituted

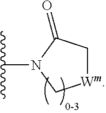

In some embodiments, R^{w} is

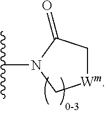

In some embodiments, R^{w} is

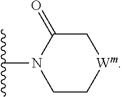

In some embodiments, R^{w} is

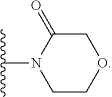 [SMILES: *N1CCOCC1=O]

In some embodiments, W^{m} is O. In some embodiments, W^{m} is S. In some embodiments, W^{m} is C(O). In some embodiments, W^{m} is NR^{◯}. In some embodiments, W^{m} is NR^{◯}, wherein R^{◯} is hydrogen or C₁₋₆ aliphatic.

In some embodiments, R^{w} is an optionally substituted 5-6 membered heteroaryl having 1-4 heteroatoms independently selected from nitrogen, oxygen, and sulfur. In some embodiments R^{w} is an optionally substituted 5 membered heteroaryl having 1-4 heteroatoms independently selected from nitrogen, oxygen, and sulfur. In some embodiments, R^{w} is an optionally substituted pyrazolyl, imidazolyl, triazolyl, or tetrazolyl. In some embodiments, R^{w} is an optionally substituted imidazolyl, optionally substituted with —C(O)N(R^{◯})₂. In some embodiments, R^{w} is an optionally substituted furanyl, thiophenyl, oxazolyl, isoxazolyl, isothiazolyl, thiazolyl, oxadiazolyl, or thiadiazolyl. In some embodiments, R^{w} is furanyl, optionally substituted with —C(O)N(R^{◯})₂.

In some embodiments R^{w} is an optionally substituted 6 membered heteroaryl having 1-4 heteroatoms independently selected from nitrogen, oxygen, and sulfur. In some embodiments R^{w} is an optionally substituted 6 membered heteroaryl having 1-4 nitrogen heteroatoms. In some embodiments, R^{w} is optionally substituted pyridinyl, pyrimidinyl, pyridazinyl, or triazinyl. In some embodiments, R^{w} is an optionally substituted pyridinonyl, pyrazinonyl, or pyrimidinoyl.

In some embodiments, R^{w} is —NHR, wherein R is an optionally substituted 5-6 membered heteroaryl having 1-4 heteroatoms independently selected from nitrogen, oxygen, and sulfur. In some embodiments, R^{w} is —NHR, wherein R is an optionally substituted 6 membered heteroaryl having 1-4 heteroatoms independently selected from nitrogen, oxygen, and sulfur.

In some embodiments, R^{w} is —S(O)₂NH₂. In some embodiments, R^{w} is —S(O)₂NHR, wherein R is an optionally substituted C₁₋₆ aliphatic. In embodiments, R^{w} is —S(O)₂NHR, wherein R is an optionally substituted phenyl, 4-7 membered saturated or partially unsaturated carbocyclic or heterocyclic ring having 1-2 heteroatoms independently selected from nitrogen, oxygen, and sulfur, or a 5-6 membered heteroaryl ring having 1-4 heteroatoms independently selected from nitrogen, oxygen, and sulfur.

In embodiments, R^{w} is —S(O)₂(NH)R. In some embodiments, R^{w} is —S(O)₂(NH)H. In some embodiments, R^{w} is —S(O)₂(NH)R, wherein R is an optionally substituted C₁₋₆ aliphatic. In some embodiments, R^{w} is —S(O)₂(NH)R, wherein R is an optionally substituted phenyl, 4-7 membered saturated or partially unsaturated carbocyclic or heterocyclic ring having 1-2 heteroatoms independently selected from nitrogen, oxygen, and sulfur, or a 5-6 membered heteroaryl ring having 1-4 heteroatoms independently selected from nitrogen, oxygen, and sulfur.

In some embodiments, R^{w} is —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, or —NRS(O)₂R. In some embodiments, R^{w} is —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R. In some embodiments, R^{w} is —S(O)R, —S(O)₂R, —S(O)(NR)R, —S(O)₂NR₂, or —NRS(O)₂R. In some embodiments, R^{w} is —C(O)R, —C(O)OR, —C(O)NR₂, —C(O)NROR, —OC(O)R, —OC(O)NR₂, —NRC(O)OR, —NRC(O)R, or —NRC(O)N(R)₂. In some embodiments, R^{w} is —S(O)R, —S(O)₂R, —S(O)(NR)R, —S(O)₂NR₂, —C(O)R, —C(O)OR, —C(O)NR₂, or —C(O)NROR. In some embodiments, R^{w} is —C(O)R, —C(O)OR, or —C(O)NR₂.

In some embodiments, R^{w} is —C(O)H. In some embodiments, R^{w} is —C(O)R, wherein R is of R^{w} is optionally substituted C₁₋₆ aliphatic. In some embodiments, R^{w} is —C(O)R, wherein R is of R^{w} is optionally substituted phenyl. In some embodiments, R^{w} is —C(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)R, wherein R is of R^{w} is an optionally substituted 5-6 membered heteroaryl ring having 1-4 heteroatoms independently selected from nitrogen, oxygen, and sulfur.

In some embodiments, R^{w} is —C(O)R, wherein R is of R^{w} is optionally substituted C₁₋₆ aliphatic. In some embodiments, R^{w} is —C(O)R, wherein R is of R^{w} is optionally substituted phenyl. In some embodiments, R^{w} is —C(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)R, wherein R is of R^{w} is an optionally substituted 5-6 membered heteroaryl ring having 1-4 heteroatoms independently selected from nitrogen, oxygen, and sulfur.

In some embodiments, R^{w} is —C(O)OH. In some embodiments, R^{w} is —C(O)OR, wherein R is of R^{w} is optionally substituted C₁₋₆ aliphatic. In some embodiments, R^{w} is —C(O)OR, wherein R is of R^{w} is optionally substituted phenyl. In some embodiments, R^{w} is —C(O)OR, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)OR, wherein R is of R^{w} is an optionally substituted 5-6 membered heteroaryl ring having 1-4 heteroatoms independently selected from nitrogen, oxygen, and sulfur.

In some embodiments, R^{w} is —C(O)NH₂. In some embodiments, R^{w} is —C(O)NR₂, wherein each R is of R^{w} is independently hydrogen or an optionally substituted C₁₋₆ aliphatic. In some embodiments, R^{w} is —C(O)NR₂, wherein each R is of R^{w} is independently hydrogen or an optionally substituted phenyl. In some embodiments, R^{w} is —C(O)NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —C(O)NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —C(O)NHR, wherein R is an optionally substituted C₁₋₆ aliphatic. In some embodiments, R^{w} is —C(O)NHR, wherein R is C₁₋₆ aliphatic. In some embodiments, R^{w} is —C(O)NHR, wherein R is methyl, ethyl, or cyclopropyl. In some embodiments, R^{w} is —C(O)NR₂, wherein the two R groups of R^{w} are taken together with their intervening atoms to form a 3-7 membered saturated or partially unsaturated monocyclic ring having 0-3 heteroatoms, in addition to the atom or adjacent atoms to which they are attached, independently selected form nitrogen, oxygen, and sulfur. In some embodiments, R^{w} is —C(O)NR₂, wherein the two R groups of R^{w} are taken together with their intervening atoms to form a 3-7 membered saturated or partially unsaturated monocyclic ring having 0 heteroatoms, in addition to the atom or adjacent atoms to which they are attached. In some embodiments, R^{w} is —C(O)NR₂, wherein the two R groups of R^{w} are taken together with their intervening atoms to form an aziridinyl, azetidinyl, diazetidinyl, pyrrolidinyl, or piperidinyl.

In some embodiments, R^{w} is —C(O)NROR, wherein each R is of R^{w} is independently hydrogen or an optionally substituted C₁₋₆ aliphatic. In some embodiments, R^{w} is —C(O)NROR, wherein each R is of R^{w} is independently hydrogen or an optionally substituted phenyl. In some embodiments, R^{w} is —C(O)NROR, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —C(O)NROR, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —OC(O)H. In some embodiments, R^{w} is —OC(O)R, wherein R is of R^{w} is optionally substituted C₁₋₆ aliphatic. In some embodiments, R^{w} is —OC(O)R, wherein R is of R^{w} is optionally substituted phenyl. In some embodiments, R^{w} is —OC(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —OC(O)R, wherein R is of R^{w} is an optionally substituted 5-6 membered heteroaryl ring having 1-4 heteroatoms independently selected from nitrogen, oxygen, and sulfur.

In some embodiments, R^{w} is —OC(O)NR₂, wherein each R is of R^{w} is independently hydrogen or an optionally substituted C₁₋₆ aliphatic. In some embodiments, R^{w} is —OC(O)NR₂, wherein each R is of R^{w} is independently hydrogen or an optionally substituted phenyl. In some embodiments, R^{w} is —OC(O)NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —OC(O)NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRC(O)OR, wherein each R is of R^{w} is independently hydrogen or an optionally substituted C₁₋₆ aliphatic. In some embodiments, R^{w} is —NRC(O)OR, wherein each R is of R^{w} is independently hydrogen or an optionally substituted phenyl. In some embodiments, R^{w} is —NRC(O)OR, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)OR, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRC(O)R, wherein each R is of R^{w} is independently hydrogen or an optionally substituted C₁₋₆ aliphatic. In some embodiments, R^{w} is —NRC(O)R, wherein each R is of R^{w} is independently hydrogen or an optionally substituted phenyl. In some embodiments, R^{w} is —NRC(O)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRC(O)N(R)₂, wherein each R is of R^{w} is independently hydrogen or an optionally substituted C₁₋₆ aliphatic. In some embodiments, R^{w} is —NRC(O)N(R)₂, wherein each R is of R^{w} is independently hydrogen or an optionally substituted phenyl. In some embodiments, R^{w} is —NRC(O)N(R)₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)N(R)₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRS(O)₂R, wherein each R is of R^{w} is independently hydrogen or an optionally substituted C₁₋₆ aliphatic. In some embodiments, R^{w} is —NRS(O)₂R, wherein each R is of R^{w} is independently hydrogen or an optionally substituted phenyl. In some embodiments, R^{w} is —NRS(O)₂R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRS(O)₂R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —S(O)H. In some embodiments, R^{w} is —S(O)R, wherein R is of R^{w} is optionally substituted C₁₋₆ aliphatic. In some embodiments, R^{w} is —S(O)R, wherein R is of R^{w} is optionally substituted phenyl. In some embodiments, R^{w} is —S(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —S(O)R, wherein R is of R^{w} is an optionally substituted 5-6 membered heteroaryl ring having 1-4 heteroatoms independently selected from nitrogen, oxygen, and sulfur.

In some embodiments, R^{w} is —S(O)₂H. In some embodiments, R^{w} is —S(O)₂R, wherein R is of R^{w} is optionally substituted C₁₋₆ aliphatic. In some embodiments, R^{w} is —S(O)₂R, wherein R is of R^{w} is optionally substituted phenyl. In some embodiments, R^{w} is —S(O)₂R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —S(O)₂R, wherein R is of R^{w} is an optionally substituted 5-6 membered heteroaryl ring having 1-4 heteroatoms independently selected from nitrogen, oxygen, and sulfur.

In some embodiments, R^{w} is —S(O)(NR)R, wherein each R is of R^{w} is independently hydrogen or optionally substituted C₁₋₆ aliphatic. In some embodiments, R^{w} is —S(O)(NR)R, wherein each R is of R^{w} is independently hydrogen or optionally substituted phenyl. In some embodiments, R^{w} is —S(O)(NR)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —S(O)(NR)R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —S(O)₂NR₂, wherein each R is of R^{w} is independently hydrogen or an optionally substituted C₁₋₆ aliphatic. In some embodiments, R^{w} is —S(O)₂NR₂, wherein each R is of R^{w} is independently hydrogen or an optionally substituted phenyl. In some embodiments, R^{w} is —S(O)₂NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —S(O)₂NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is an optionally substituted 3-7 membered saturated or partially unsaturated carbocyclic or heterocyclic ring having 1-2 heteroatoms independently selected from nitrogen, oxygen, and sulfur. In some embodiments, R^{w} is an optionally substituted 3-7 membered saturated or partially unsaturated heterocyclic ring having 1-2 heteroatoms independently selected from nitrogen, oxygen, and sulfur, and is substituted with ═O. In some embodiments, R^{w} is an optionally substituted 5-6 membered monocyclic heteroaryl ring having 1-4 heteroatoms independently selected from nitrogen, oxygen, and sulfur. In some embodiments, R^{w} is an optionally substituted 5-6 membered monocyclic heteroaryl ring having 1-4 heteroatoms independently selected from nitrogen, oxygen, and sulfur, and is substituted with ═O. In some embodiments, R^{w} is an optionally substituted:

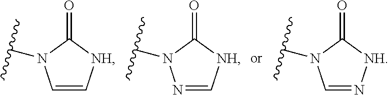

In some embodiments, R^{w} is:

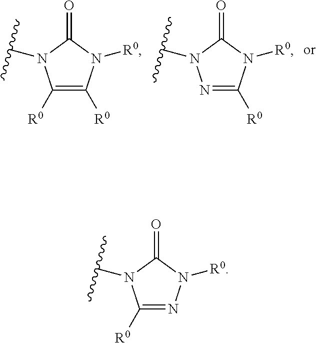

In some such embodiments, R^{◯} is hydrogen or C₁₋₆ aliphatic.

In some embodiments, R^{w} is:

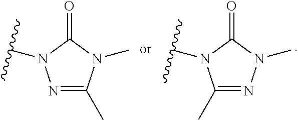

In some embodiments, R^{w} is an optionally substituted:

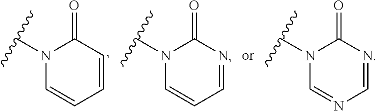

In some embodiments, R^{w} is:

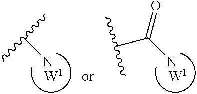

wherein Ring W¹ is an optionally substituted 3-7 membered saturated or partially unsaturated heterocyclic ring having 1 additional heteroatom selected from nitrogen, oxygen, and sulfur, or a 5-6 membered monocyclic heteroaryl ring having 1-3 additional heteroatoms independently selected from nitrogen, oxygen, and sulfur.

As defined above and described herein, Ring W¹ is an optionally substituted 3-7 membered saturated or partially unsaturated heterocyclic ring having 1 additional heteroatom selected from nitrogen, oxygen, and sulfur, or a 5-6 membered monocyclic heteroaryl ring having 1-3 additional heteroatoms independently selected from nitrogen, oxygen, and sulfur. In some embodiments, Ring W¹ is an optionally substituted 3-7 membered saturated or partially unsaturated heterocyclic ring having 1 additional heteroatom selected from nitrogen, oxygen, and sulfur. In some embodiments, Ring W¹ is an optionally substituted 5-6 membered saturated or partially unsaturated heterocyclic ring having 1 additional heteroatom selected from nitrogen, oxygen, and sulfur. In some embodiments, Ring W¹ is an optionally substituted 5-6 membered monocyclic heteroaryl ring having 1-3 additional heteroatoms independently selected from nitrogen, oxygen, and sulfur. In some embodiments, Ring W¹ is an optionally substituted 5 membered monocyclic heteroaryl ring having 1-3 additional heteroatoms independently selected from nitrogen, oxygen, and sulfur. In some embodiments, Ring W¹ is an optionally substituted 6 membered monocyclic heteroaryl ring having 1-3 additional heteroatoms independently selected from nitrogen, oxygen, and sulfur.

In some embodiments, R^{w} is:

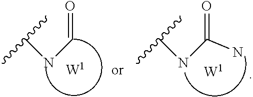

In some embodiments, R^{w} is:

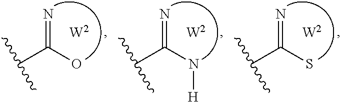

wherein Ring W² is an optionally substituted 3-7 membered partially unsaturated heterocyclic ring, or a 5-6 membered monocyclic heteroaryl ring having 1-2 additional heteroatoms independently selected from nitrogen, oxygen, and sulfur.

As defined above and described herein, Ring W² is an optionally substituted 3-7 membered partially unsaturated heterocyclic ring, or a 5-6 membered monocyclic heteroaryl ring having 1-2 additional heteroatoms independently selected from nitrogen, oxygen, and sulfur. In some embodiments, Ring W² is an optionally substituted 3-7 membered partially unsaturated heterocyclic ring. In some embodiments, Ring W² is an optionally substituted 5-6 membered partially unsaturated heterocyclic ring. In some embodiments, Ring W² is an optionally substituted 5-6 membered monocyclic heteroaryl ring having 1-2 additional heteroatoms independently selected from nitrogen, oxygen, and sulfur. In some embodiments, Ring W² is a 5 membered monocyclic heteroaryl ring having 1-2 additional heteroatoms independently selected from nitrogen, oxygen, and sulfur. In some embodiments, Ring W² is an optionally substituted oxazolyl, imidazolyl, thiazolyl, 1,3,4-thiadiazolyl, 2-imidazolinyl, 1,2,4-triazolyl, 1,2,4-oxadiazolyl, or 1,3,4-oxadiazolyl.

In some embodiments, R^{w} is an optionally substituted ring selected from:

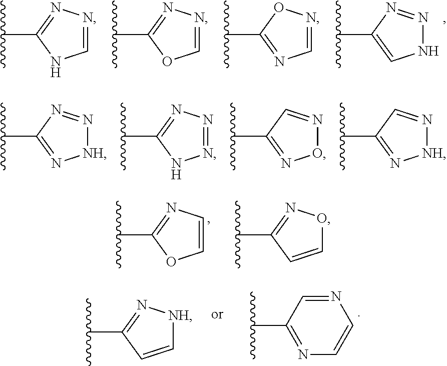

In some embodiments R^{w} is:

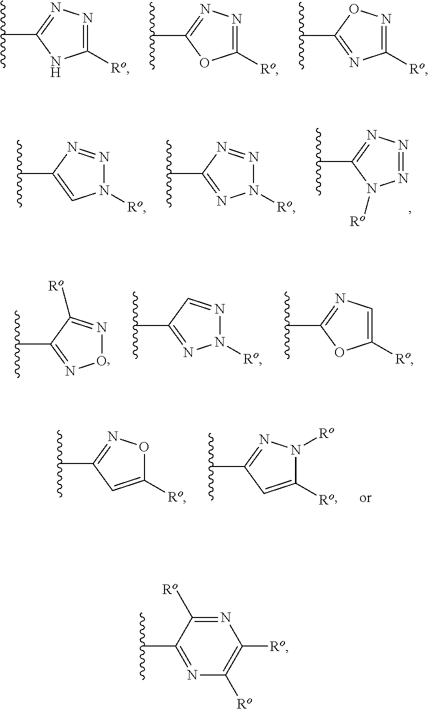

wherein each R^{◯} is a defined above and described herein (e.g., hydrogen or C₁₋₆ aliphatic).

In some embodiments, R^{w} is:

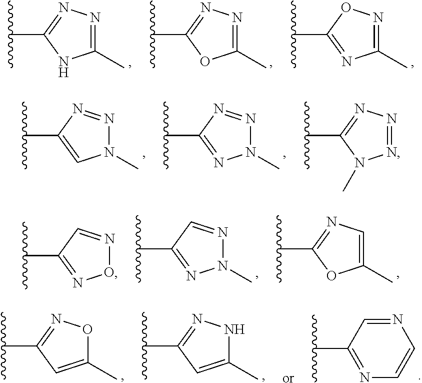

In some embodiments R^{w} is an optionally substituted 7-11 membered bicyclic heterocyclic ring having 1-4 heteroatoms independently selected from nitrogen, oxygen, and sulfur. In some embodiments R^{w} is an optionally substituted 9-10 membered bicyclic heterocyclic ring having 1-4 heteroatoms independently selected from nitrogen, oxygen, and sulfur. In some embodiments R^{w} is an optionally substituted benzo[d][1,3]dioxolyl. In some embodiments R^{w} is an optionally substituted an 11 membered bicyclic heterocyclic ring having 1-4 heteroatoms independently selected from nitrogen, oxygen, and sulfur.

In some embodiments R^{w} is an optionally substituted 8-11 membered bicyclic aryl or heteroaryl having 1-4 heteroatoms independently selected from nitrogen, oxygen and sulfur. In some embodiments, R^{w} is an optionally substituted naphthalenyl. In some embodiments, R^{w} is an optionally substituted dihydrobenzodioxepinyl. In some embodiments, R^{w} is an optionally substituted indenyl or dihydroindenyl.

In some embodiments, R^{w} is an optionally substituted 9-10 membered bicyclic heteroaryl having 1-4 heteroatoms independently selected from nitrogen, oxygen and sulfur.

In some embodiments R^{w} is an optionally substituted 9-membered bicyclic heteroaryl having 1-4 heteroatoms independently selected from nitrogen, oxygen and sulfur. In some embodiments R^{w} is an optionally substituted indolyl, benzofuranyl, benzothiophenyl, benzimidazolyl, benzoxazolyl, thienopyridinyl, pyrrolo[3,2-b]pyridinyl, pyrrolo[2,3,-b]pyridinyl, pyrazolyl[1,5-a]pyridinyl, or imidazo[1,2-a]pyridinyl, azaindazolyl (e.g., 4-, 5-, 6-, or 7-azaindazolyl), pyrrolol[2,3-c]pyridinyl or indolizinyl.

In some embodiments R^{w} is an optionally substituted 10-membered bicyclic heteroaryl having 1-4 heteroatoms independently selected from nitrogen, oxygen and sulfur. In some embodiments R^{w} is an optionally substituted quinolinyl, isoquinolinyl, quinolizinyl, quinoxalinyl, phthalazinyl, quinazolinyl, cinnolinyl, or 1,8-naphthyridinyl.

In some embodiments, R^{w} is fluoro, chloro, —CN, —OH, —OMe, —OCH₂CO₂Me, —CO₂H, —C(O)NH₂, —C(O)NHMe, —C(O)NHEt, —C(O)NHnPr, —C(O)NHCH₂CH₂OH, —C(O)NMe₂, —C(O)N(Me)Et, —C(O)N(Me)nPr, —CH₂NHMe,

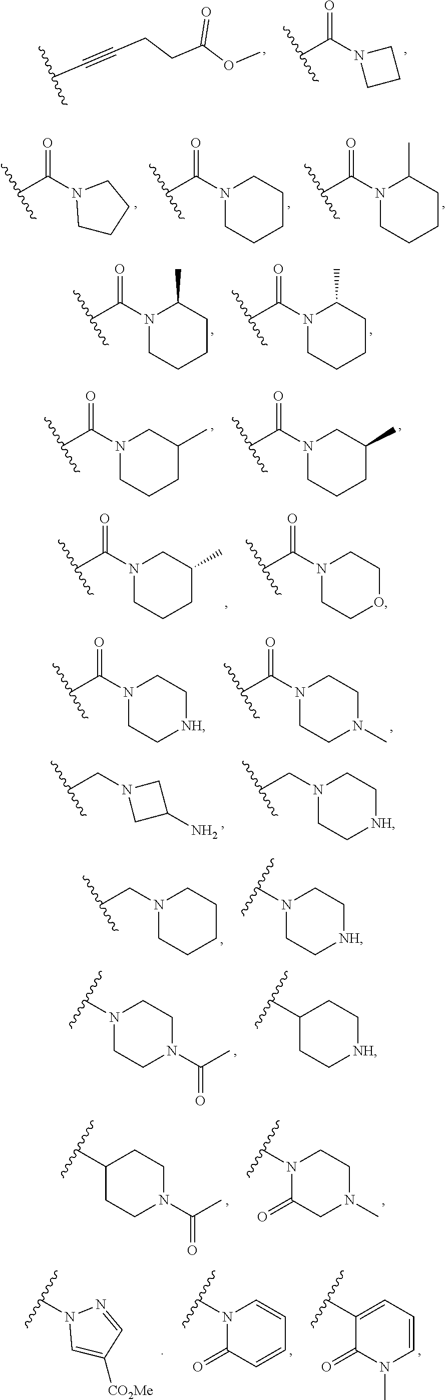

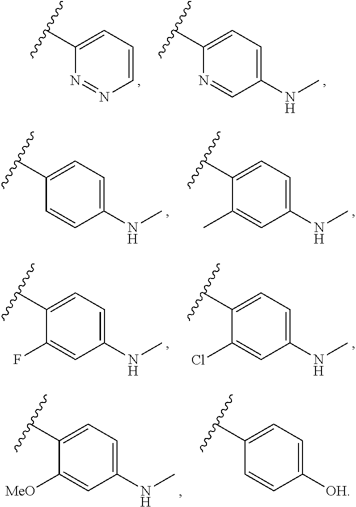

In some embodiments, R^{w} is fluoro, chloro, —CN, methyl, —CF₃, —CHF₂, —OH, —OMe, —OCH₂CO₂Me, —CO₂H, —C(O)NH₂, —C(O)NHMe, —C(O)NHEt, —C(O)NHnPr, —C(O)NHCH₂CH₂OH, —C(O)NMe₂, —C(O)N(Me)Et, —C(O)N(Me)nPr, —CH₂NHMe,

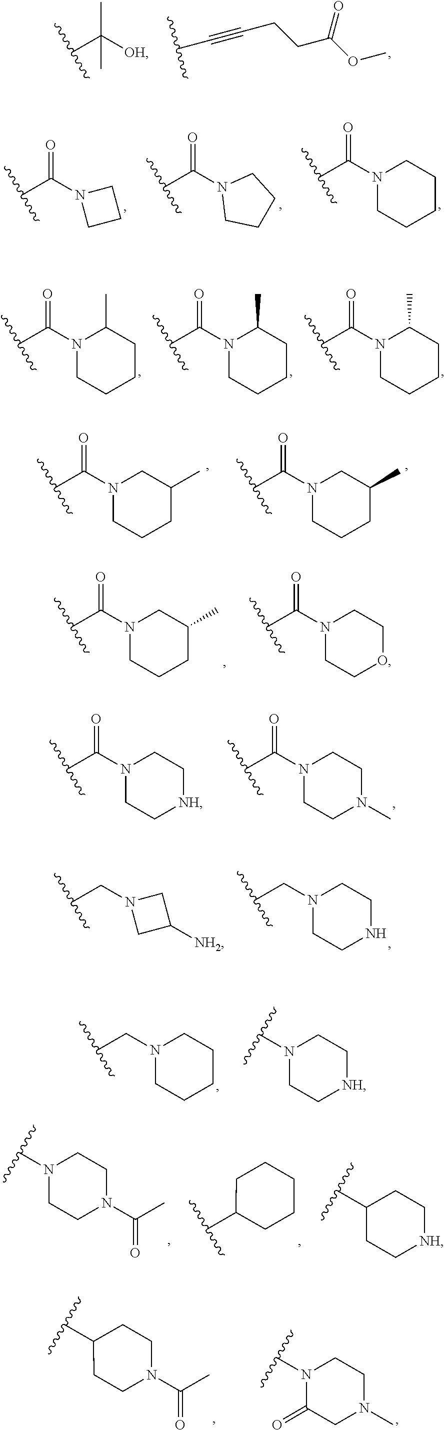

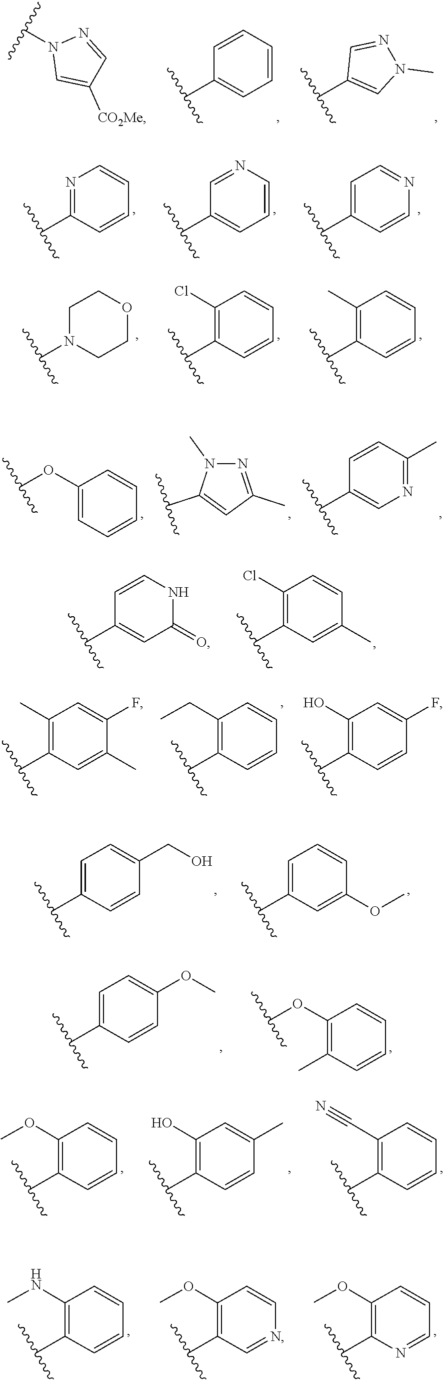

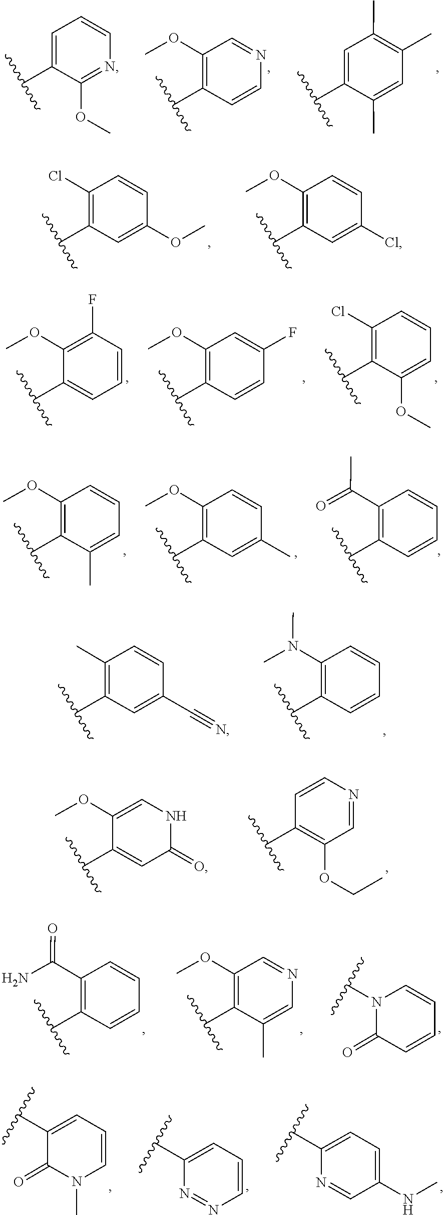

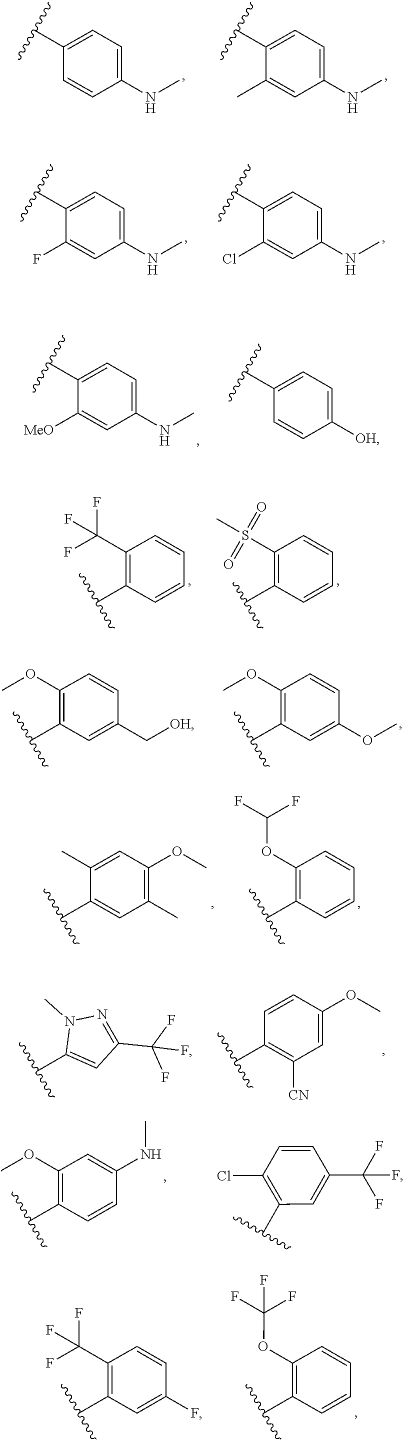

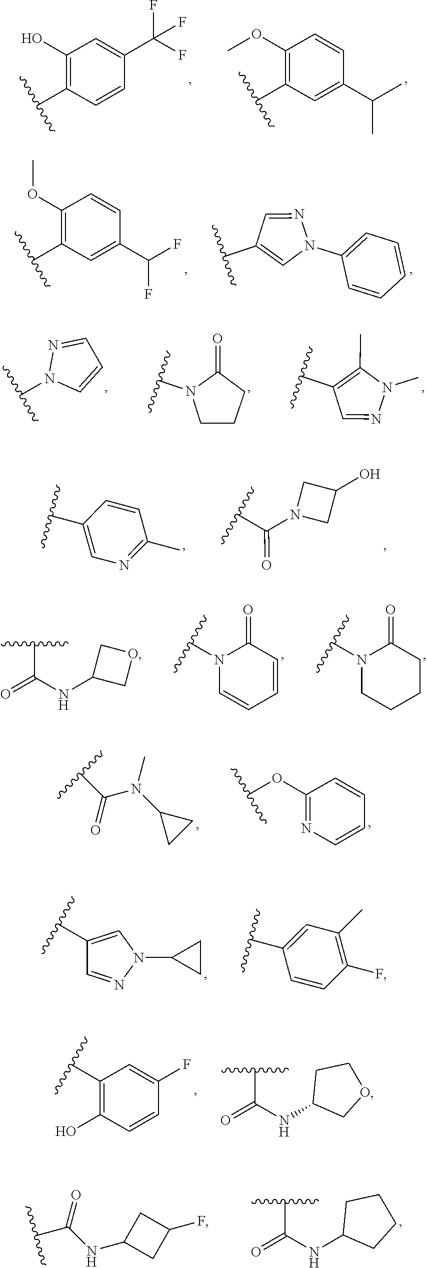

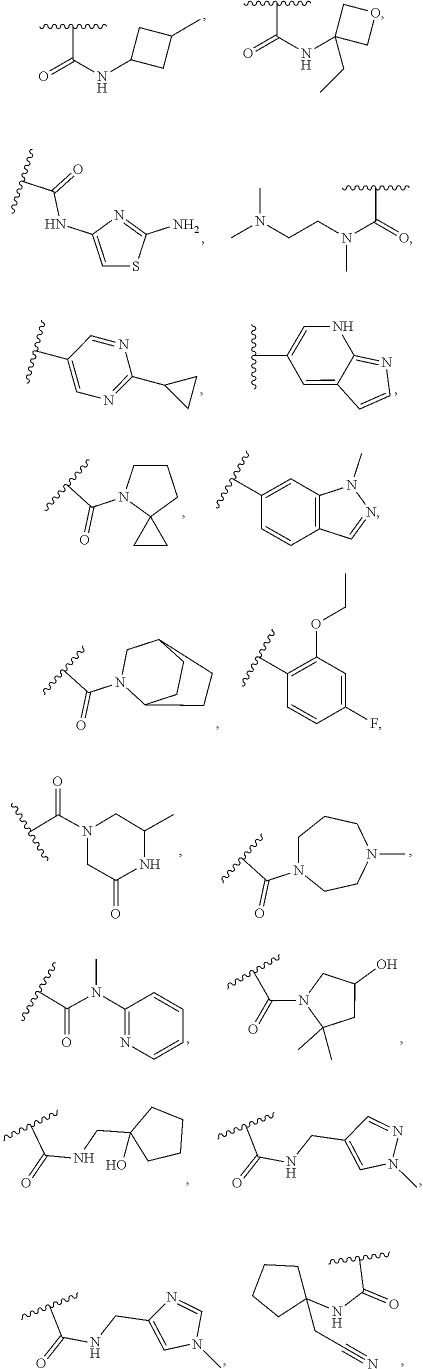

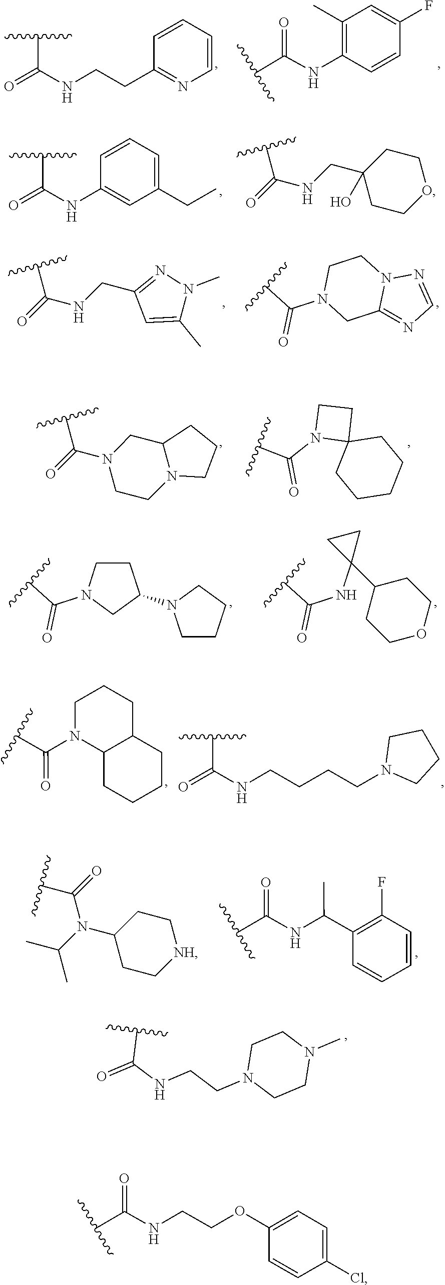

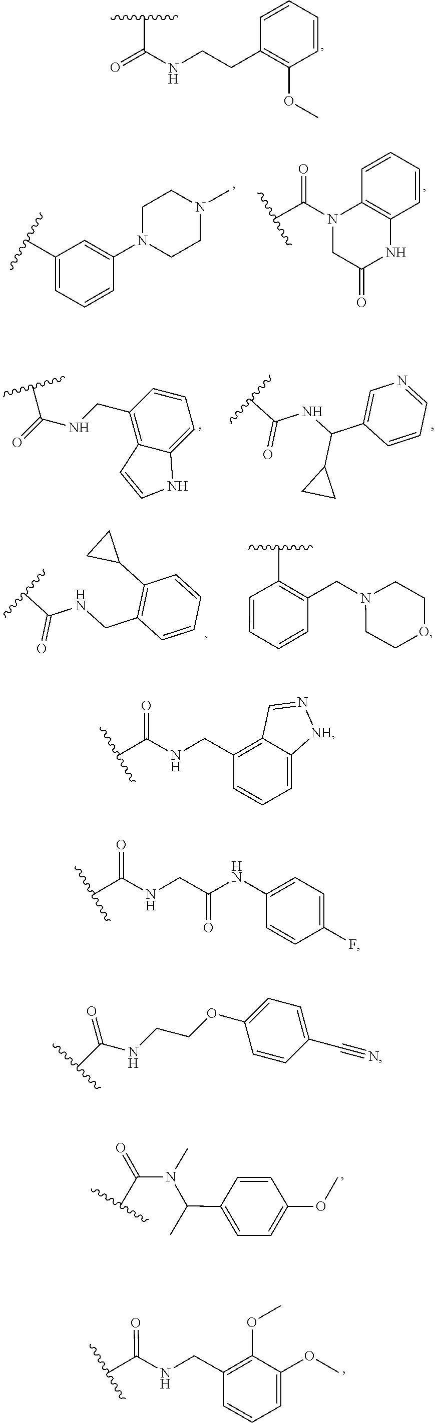

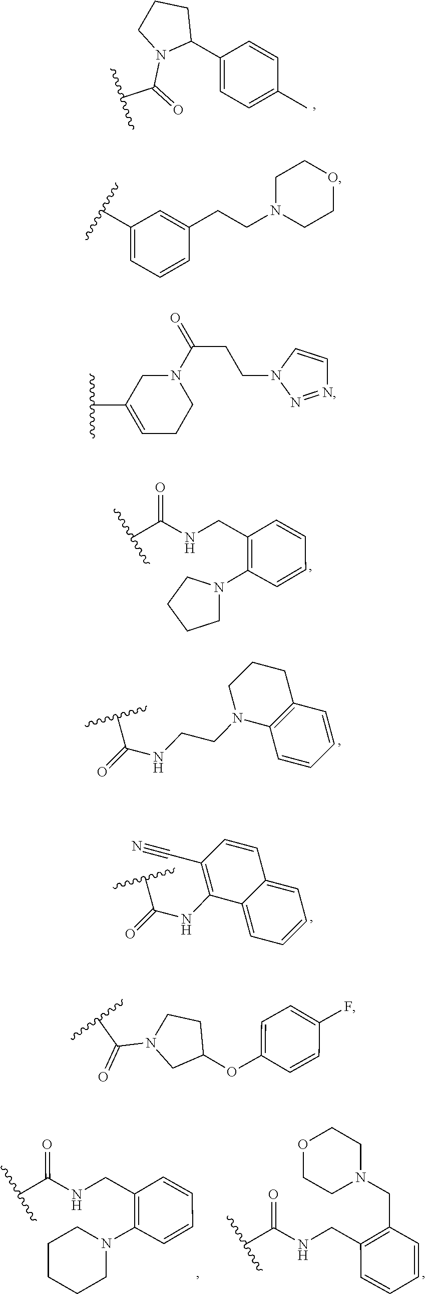

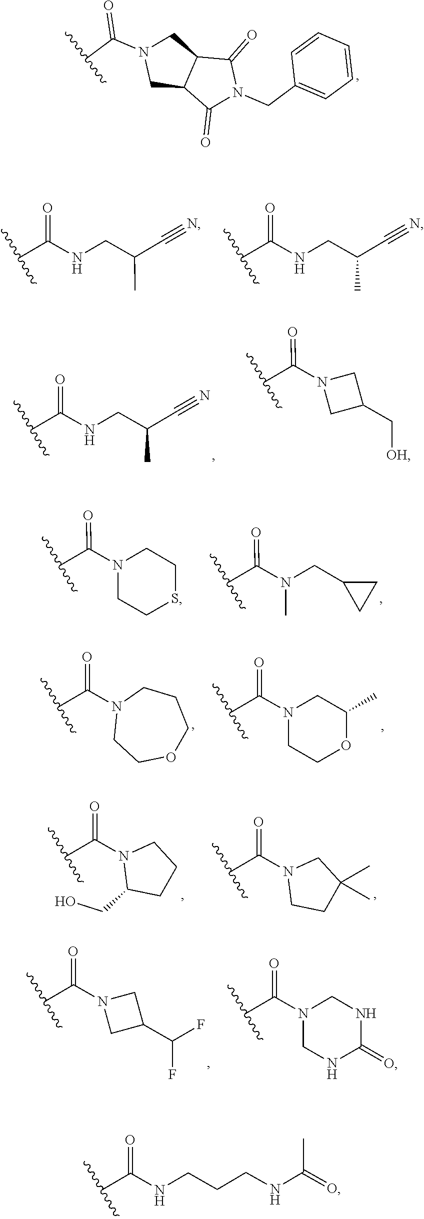

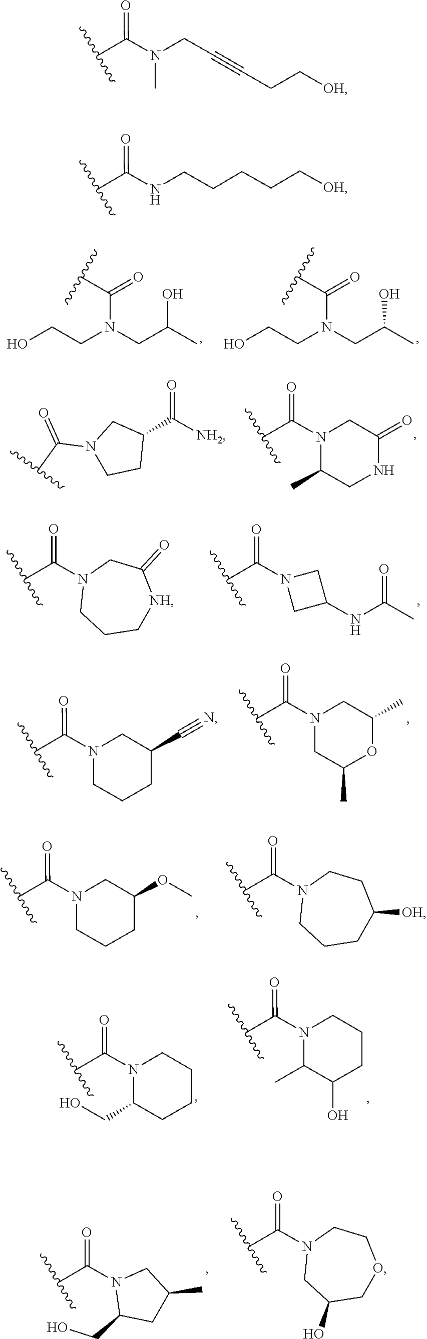

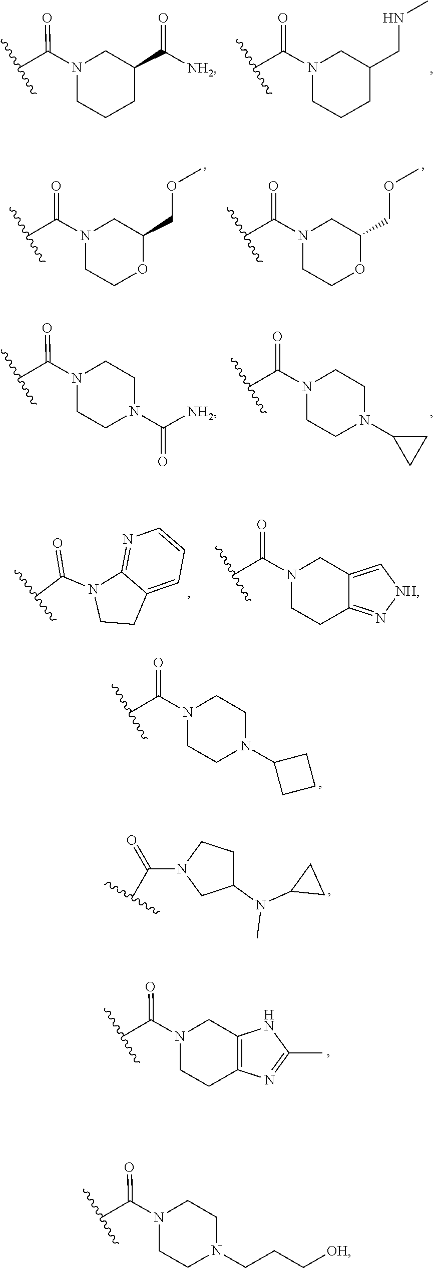

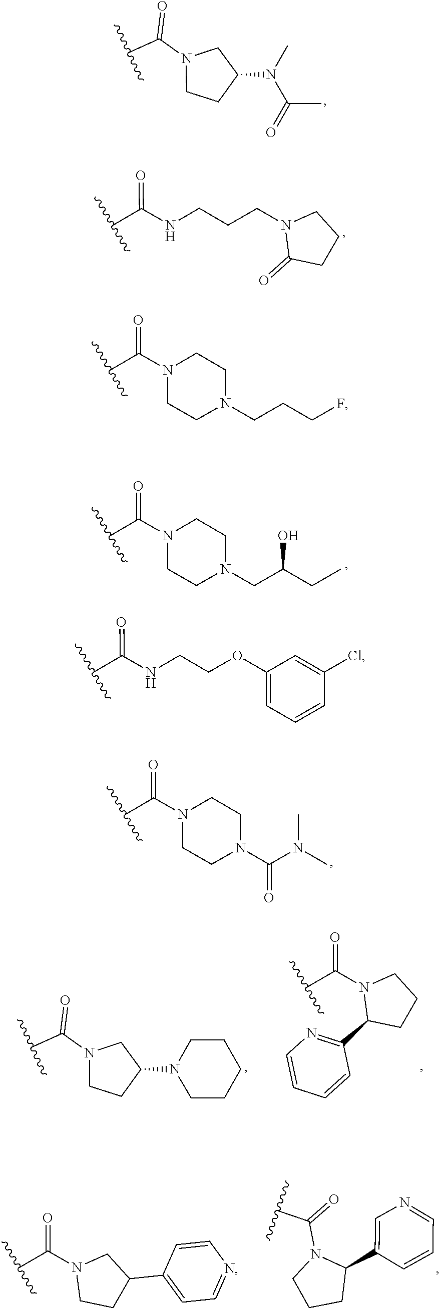

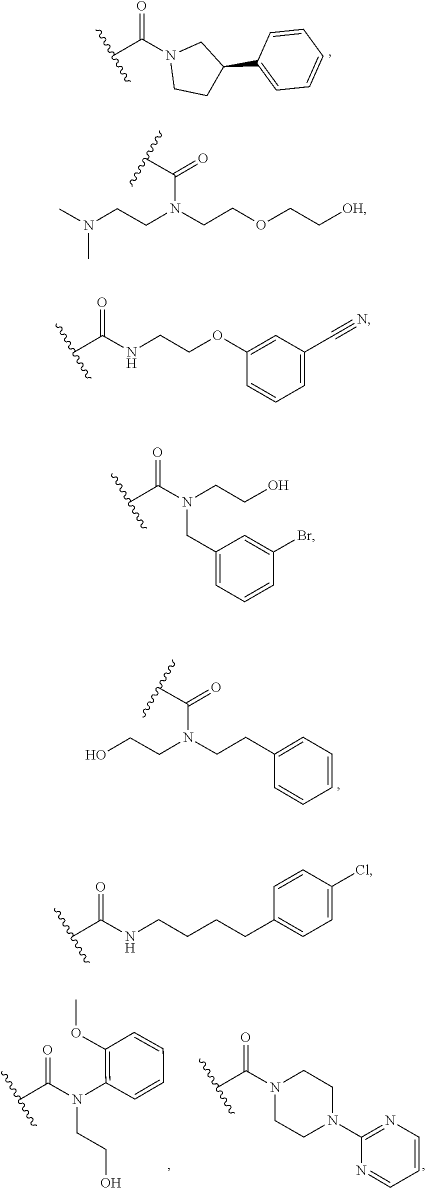

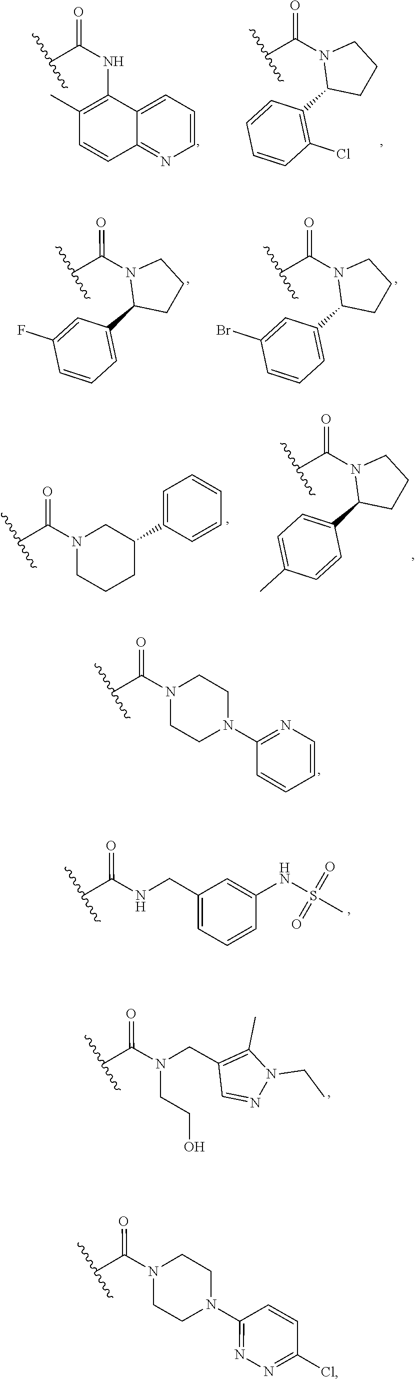

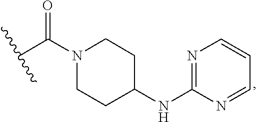 [SMILES: *C(=O)N1CCC(Nc2ncccn2)CC1]

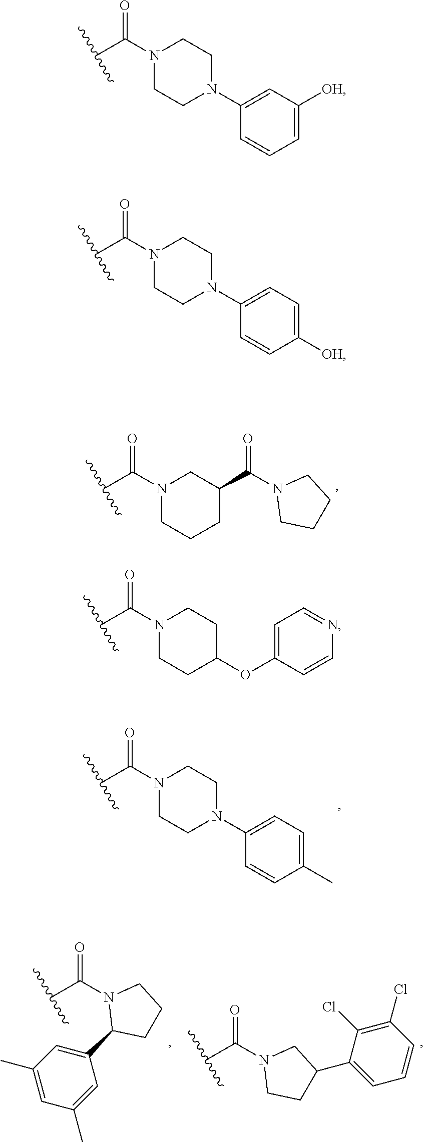

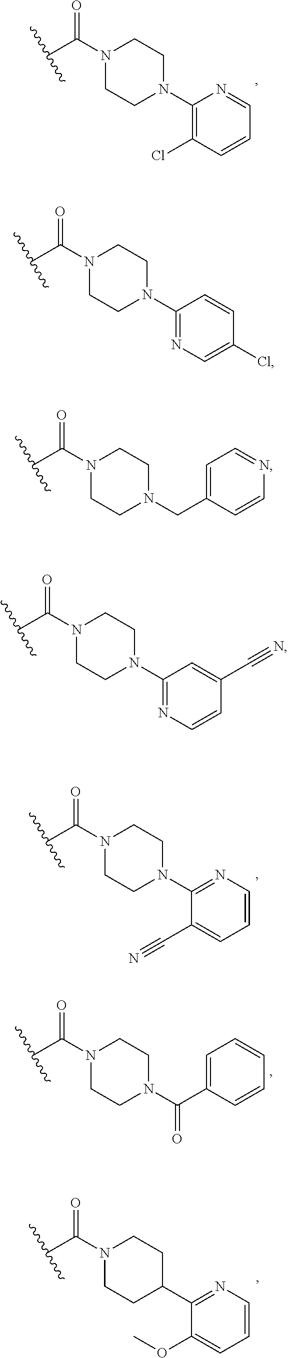

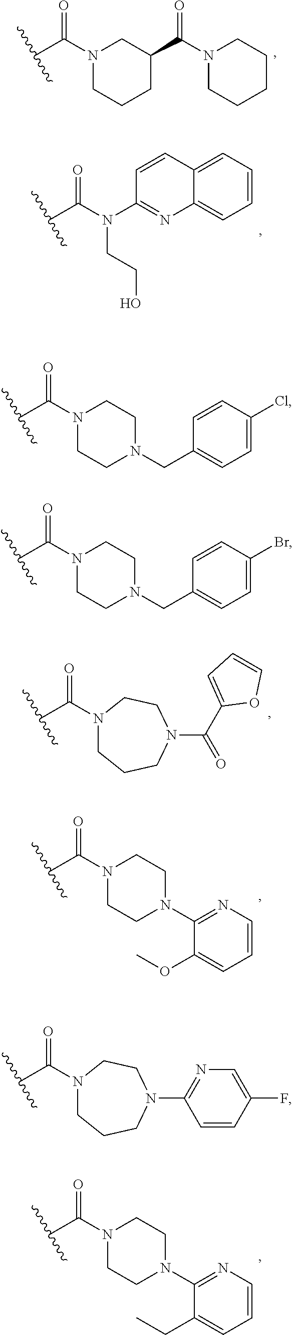

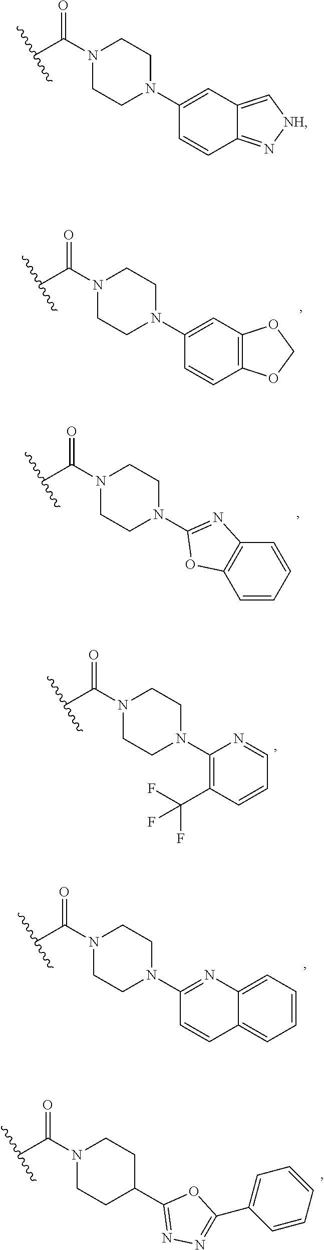 [SMILES: *C(=O)N1CCN(c2ccc3n[nH]cc3c2)CC1 ; *C(=O)N1CCN(c2ccc3c(c2)OCO3)CC1 ; *C(=O)N1CCN(c2nc3ccccc3o2)CC1 ; *C(=O)N1CCN(c2ncccc2C(F)(F)F)CC1 ; *C(=O)N1CCN(c2ccc3ccccc3n2)CC1 ; *C(=O)N1CCC(c2nnc(-c3ccccc3)o2)CC1]

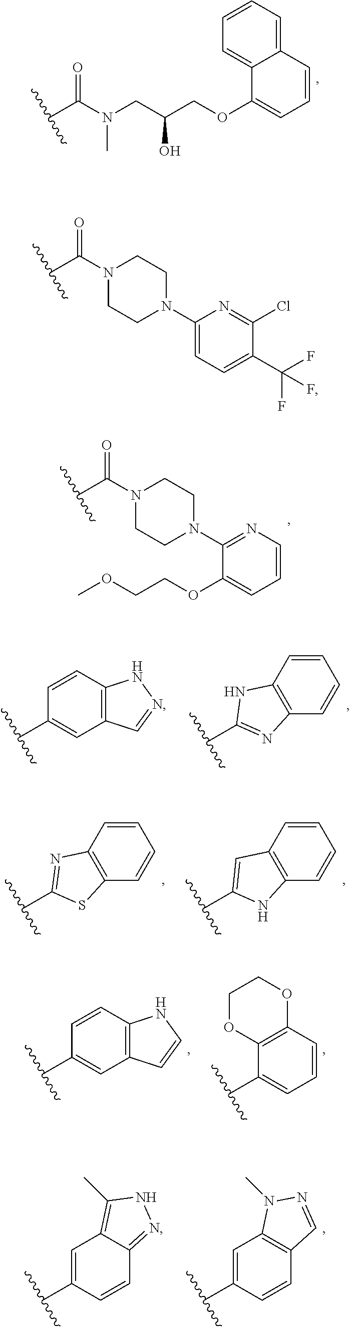

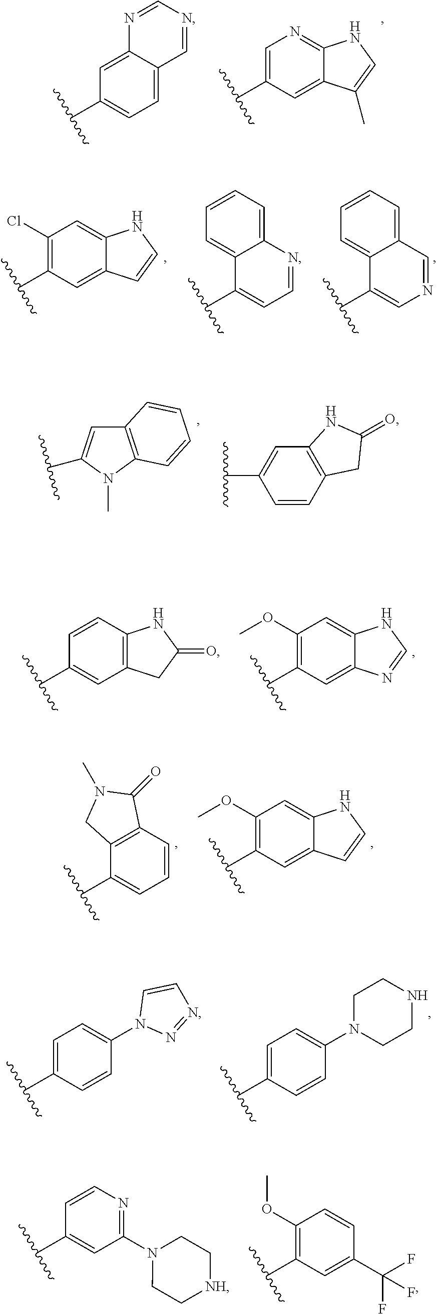

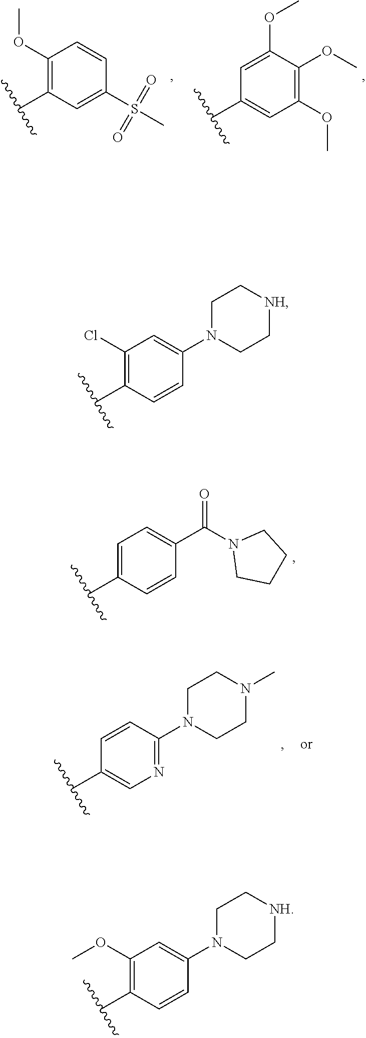 [SMILES: *c1cc(S(C)(=O)=O)ccc1OC ; *c1cc(OC)c(OC)c(OC)c1 ; *c1ccc(N2CCNCC2)cc1Cl ; *c1ccc(C(=O)N2CCCC2)cc1 ; *c1ccc(N2CCN(C)CC2)nc1 ; *c1ccc(N2CCNCC2)cc1OC]

In some embodiments, R^{w} is

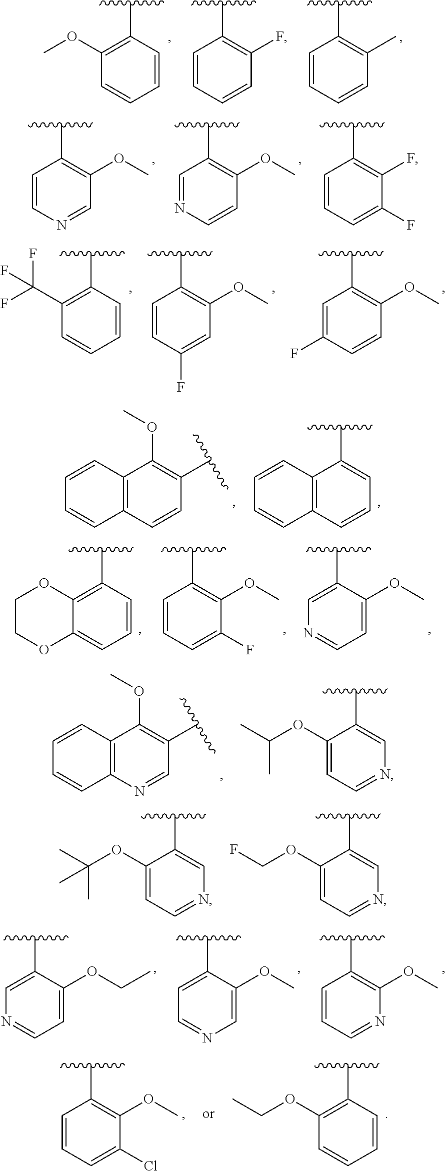

In some embodiments, R^{w} is —S(O)₂NH₂, —S(O)₂N(CH₃)₂, —S(O)(NH)CH₃,

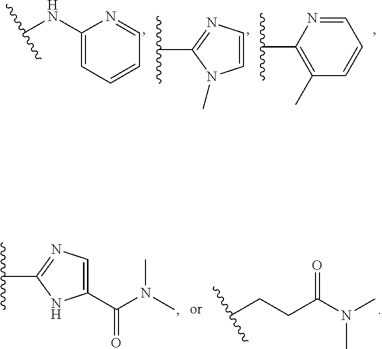

In some embodiments, R^{w} is

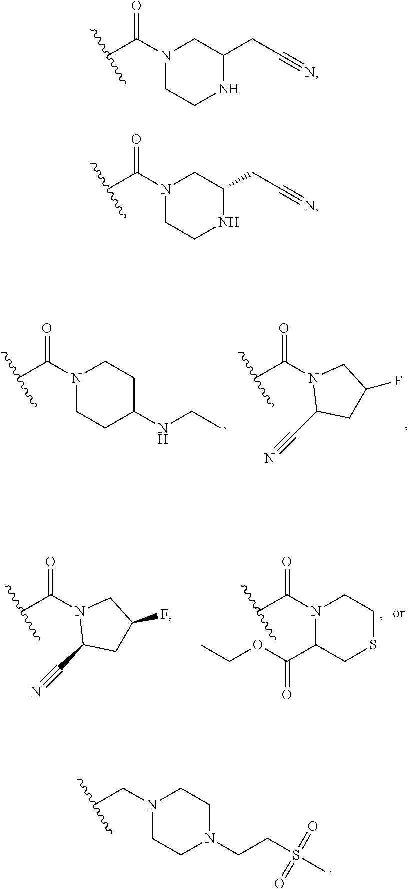

In some embodiments, R^{w} is

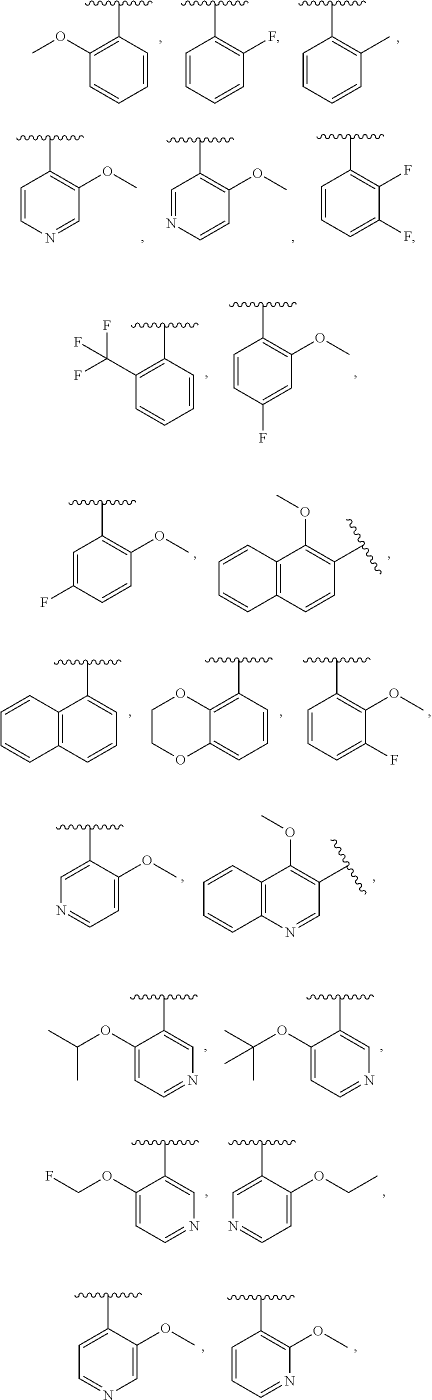

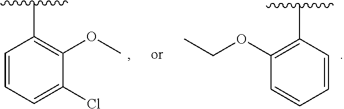

In some embodiments, R^{w} is —CH₂CH₂CH₃,

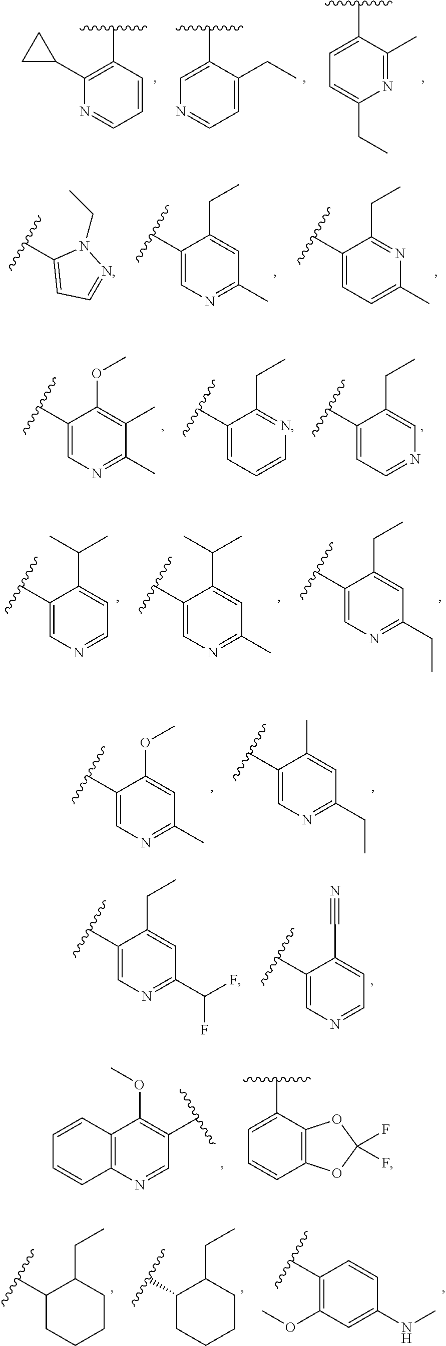

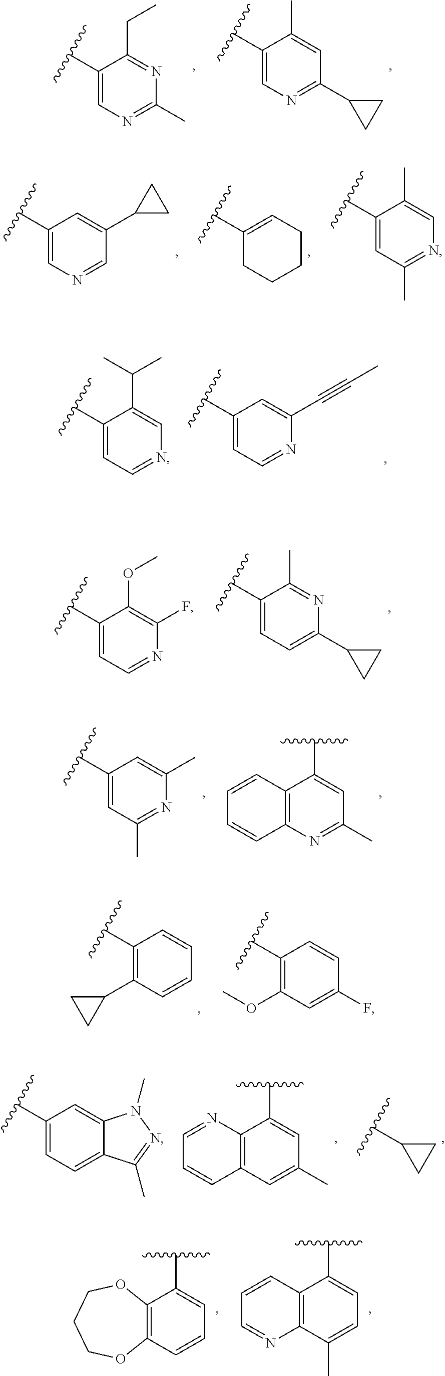

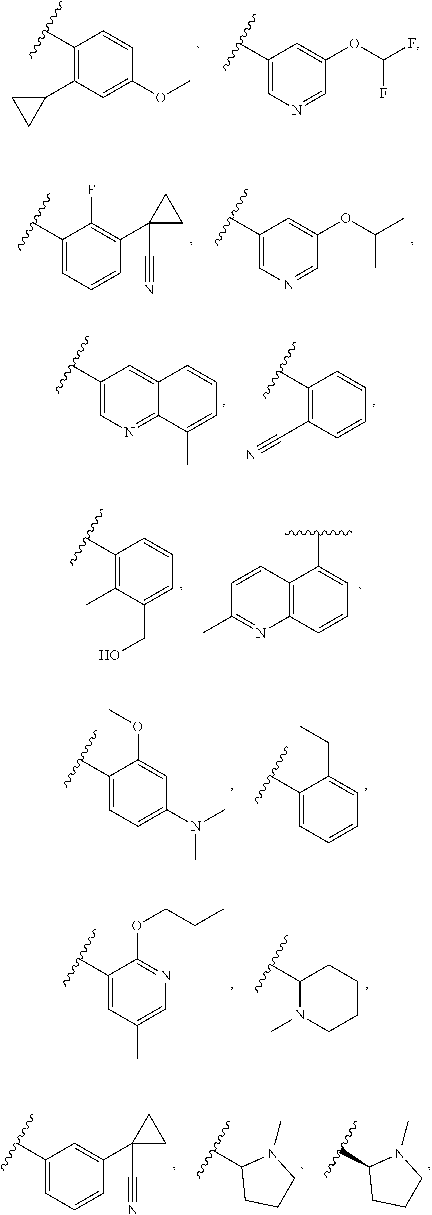

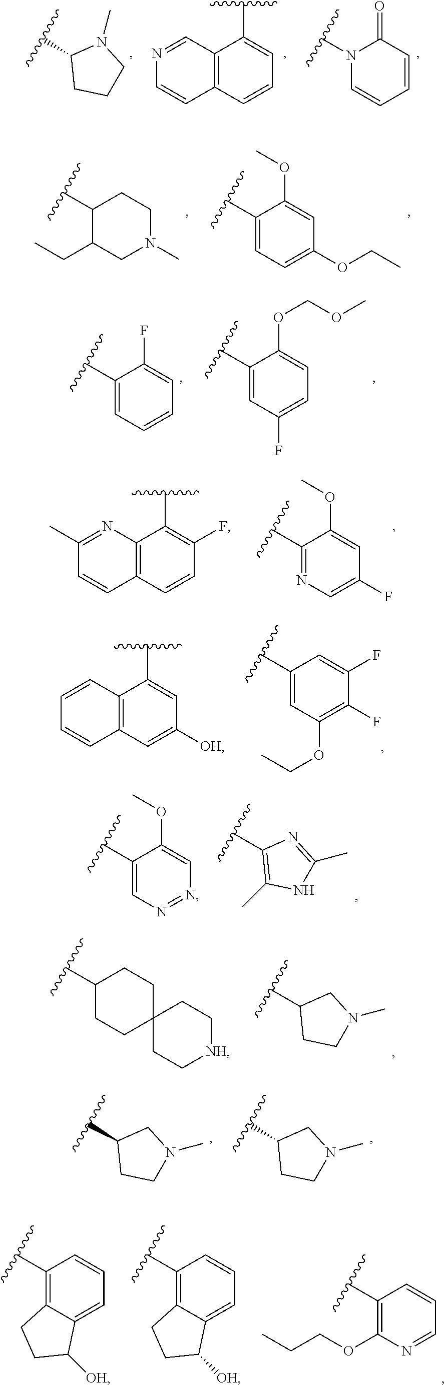

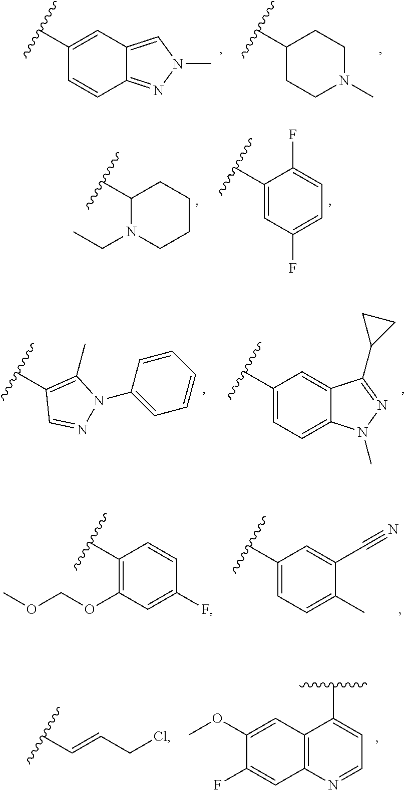

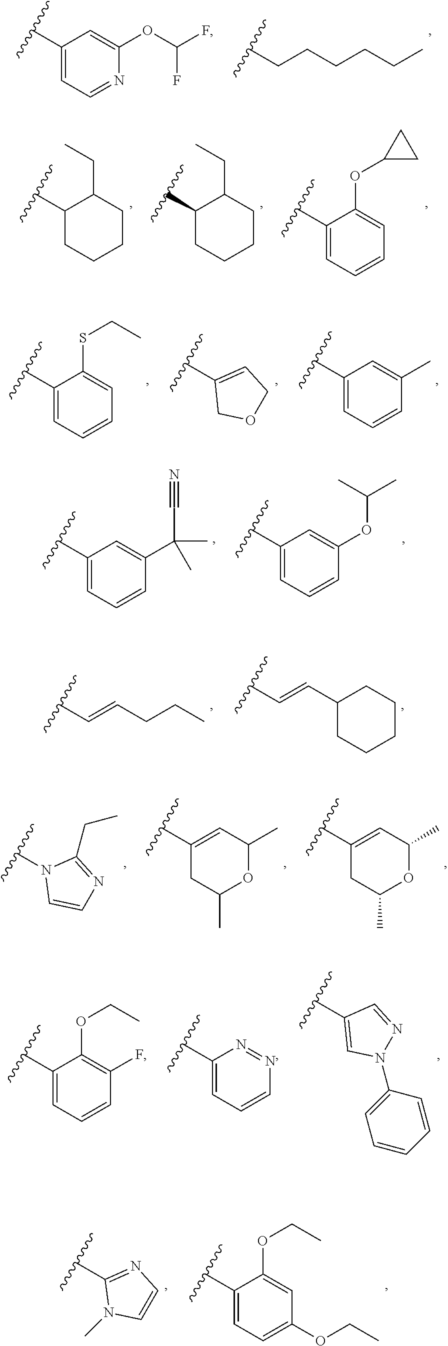

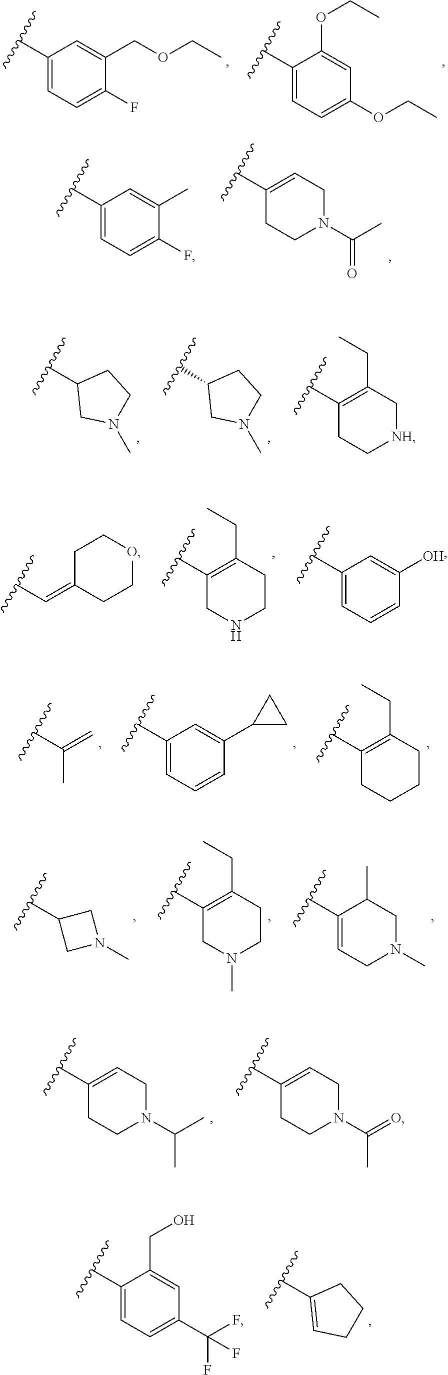

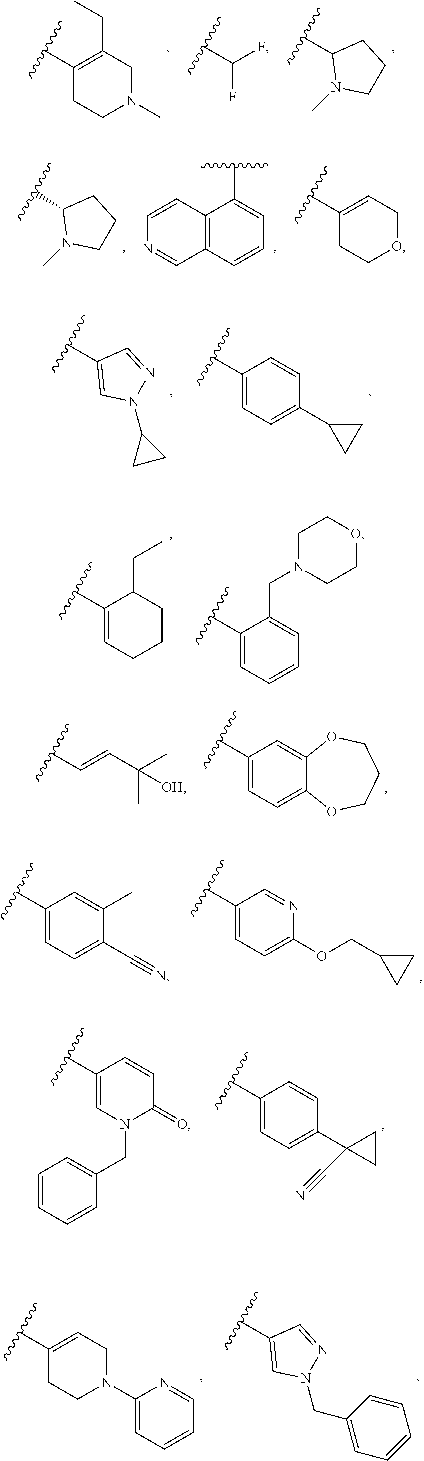

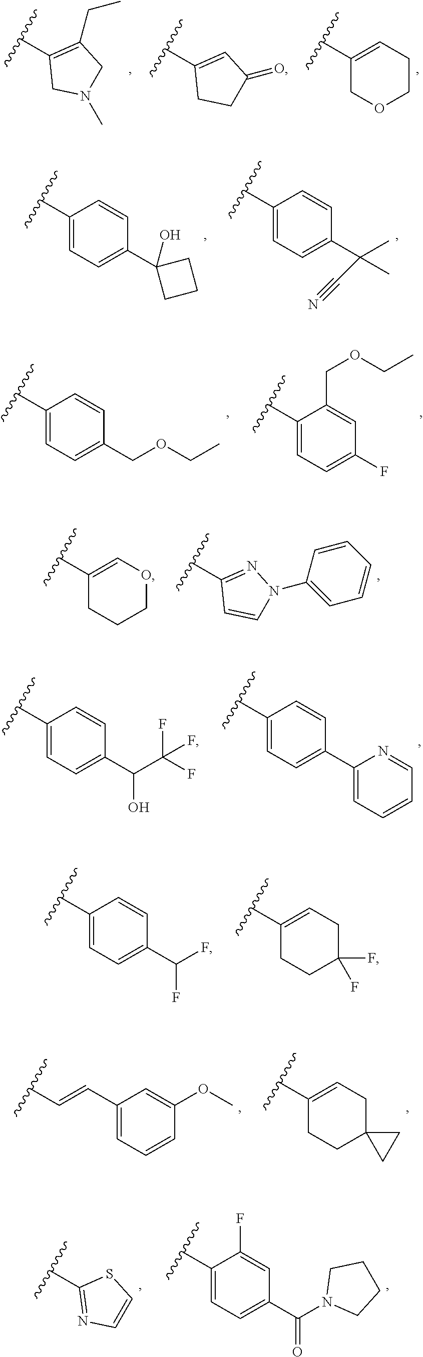

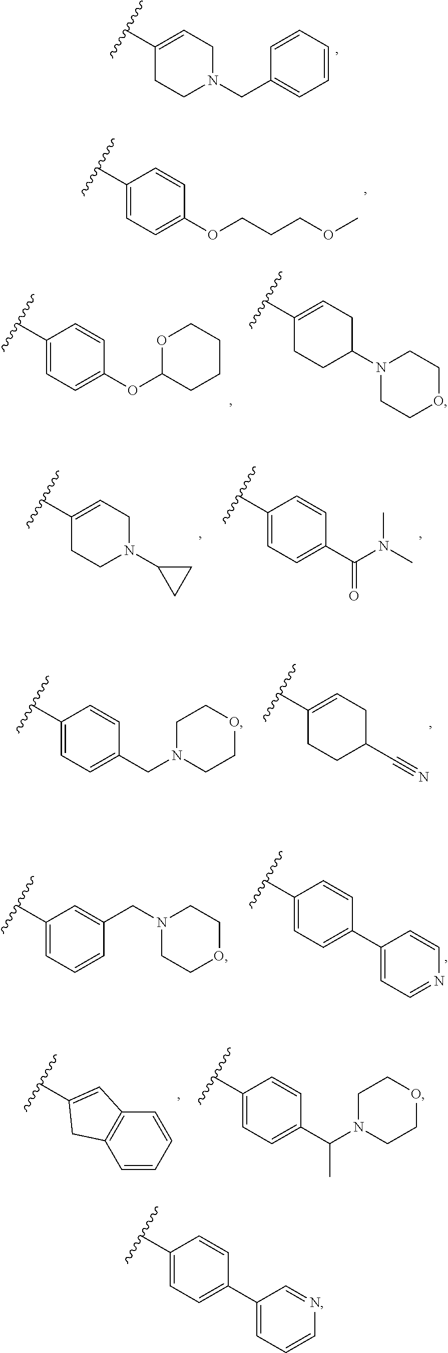

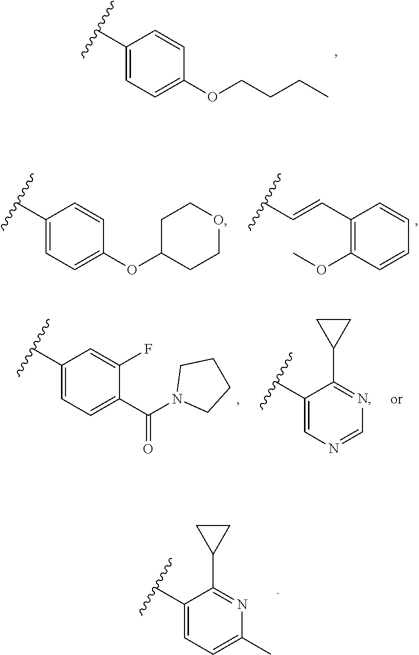

In some embodiments, R^{w} is

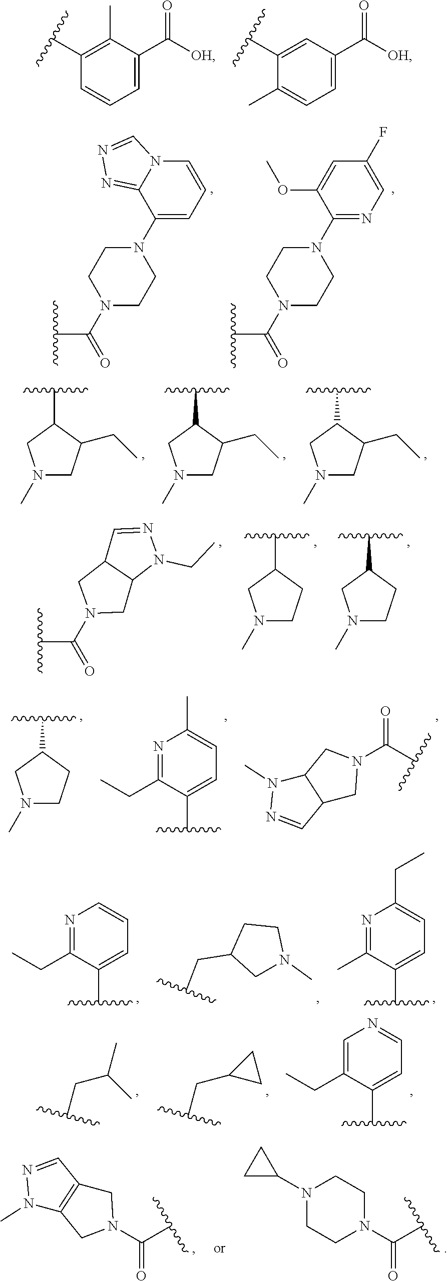

In some embodiments, R^{w} is —C(O)NH₂. In some embodiments, R^{w} is —C(O)NR₂, wherein each R is of R^{w} is independently hydrogen or an optionally substituted C₁₋₆ aliphatic. In some embodiments, R^{w} is —C(O)NR₂, wherein each R is of R^{w} is independently hydrogen or an optionally substituted phenyl. In some embodiments, R^{w} is —C(O)NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —C(O)NR₂, wherein each R is of R^{w} is independently hydrogen or an optionally substituted 5-6 membered heteroaryl ring having 1-4 heteroatoms independently selected from nitrogen, oxygen, and sulfur.

In some embodiments. R^{w} is

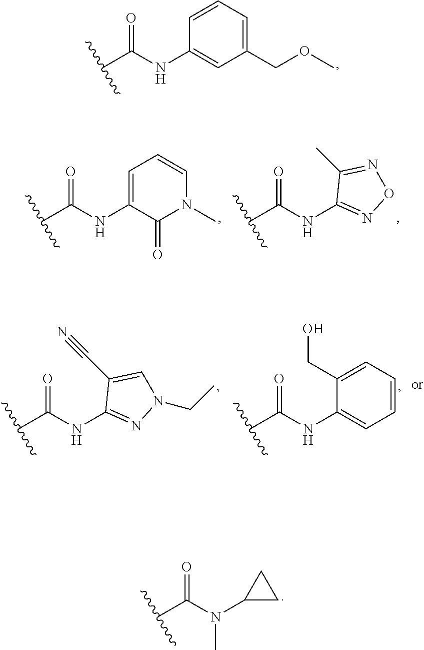

In some embodiments, each R is independently selected from R^{A}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, R^{x} is —CO₂Me, or —CH₂OH.

In some embodiments, R^{x} is —C(O)R or —C(O)OR. In some embodiments, R^{x} is —C(O)R. In some embodiments, R^{x} is —C(O)R, wherein R of R^{x} is an optionally substituted C₁₋₆ aliphatic. In some embodiments, R^{x} is —C(O)R, wherein R of R^{x} is C₁₋₆ aliphatic optionally substituted with —C(O)OR^{◯}, —OR^{◯}, halogen (e.g., fluoro), or ═O. In some embodiments, R^{x} is —C(O)OR. In some embodiments, R^{x} is —C(O)OR, wherein R of R^{x} is an optionally substituted C₁₋₆ aliphatic.

In some embodiments, R^{x} is

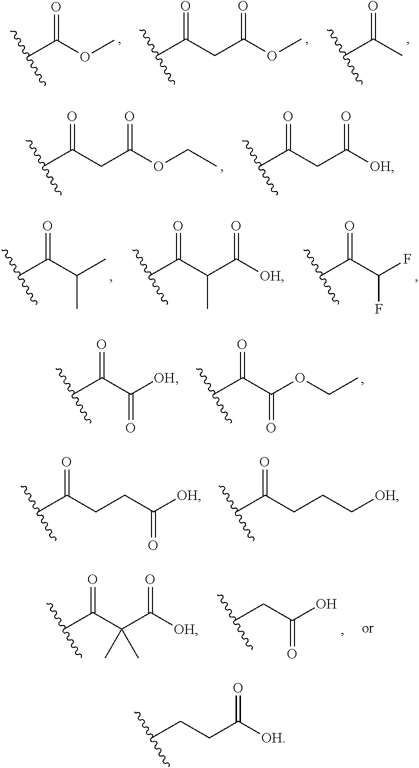

In some embodiments, R^{x} is optionally substituted C₁₋₆ aliphatic. In some embodiments, R^{x} is C₁₋₆ aliphatic optionally substituted with —OR^{◯}, wherein R^{◯} is hydrogen or C₁₋₆ aliphatic.

In some embodiments, R^{x} is —CO₂Me, —CH₂OH, —C(O)Me, —C(O)Et, —C(O)iPr, —C(O)cyclopropyl, —C(O)oxetanyl, —C(O)tetrahydropyranyl, or pyridyl.

In some embodiments, R^{x} is fluoro. In some embodiments, R^{x} is —S(O)₂CH₃. In some embodiments, R^{x} is —C(O)N(CH₃)₂.

In some embodiments, R^{x} is —C(O)R, wherein R of R^{x} is optionally substituted C₁₋₆ aliphatic. In some embodiments, R^{x} is —C(O)R, wherein R of R^{x} is C₁₋₆ aliphatic. In some embodiments, R^{x} is —C(O)R, wherein R of R^{x} is methyl, ethyl, n-propyl, or isopropyl. In some embodiments, R^{x} is —C(O)R, wherein R of R is C₁₋₆ aliphatic substituted with —OR^{◯} or —C(O)OR^{◯}, wherein R^{◯} is hydrogen or C₁₋₆ aliphatic. In some embodiments, R^{x} is —C(O)R, wherein R of R^{x} is C₁₋₆ aliphatic substituted with —OR^{◯}, wherein R^{◯} is hydrogen or C₁₋₆ aliphatic. In some embodiments, R^{x} is —C(O)R, wherein R of R^{x} is C₁₋₆ aliphatic substituted with —C(O)OR^{◯}, wherein R^{◯} is hydrogen or C₁₋₆ aliphatic.

In some embodiments, R^{x} is

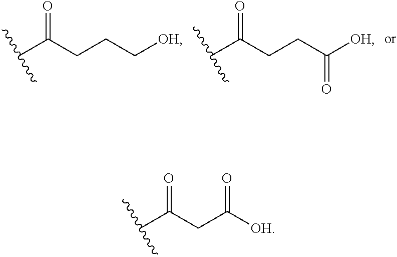 [SMILES: *C(=O)CCCO ; *C(=O)CCC(=O)O ; "or" ; *C(=O)CC(=O)O]

In some embodiments, each R^{y} is independently selected from R^{A}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, R^{y} is fluoro, chloro, bromo, iodo, methyl, ethyl, cyclopropyl, —CF₃, —CN, CH₂O, —CO₂H, —CO₂Me, —CO₂tBu, —C(O)Me, —NH₂, —NHMe, —NHAc, —NHC(O)Et, —OH, —OMe, —OCH₂CH₂NH₂, —CH₂OH, —CH₂OMe, —CH₂NHMe, —CH₂NHAc, —CH₂SO₂Me, —SO₂Me, —SO₂NH₂, —SO₂NHMe,

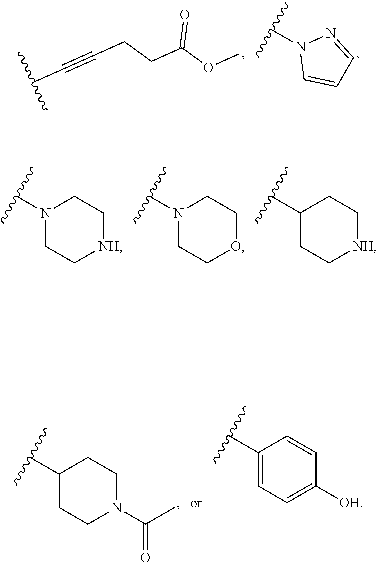

In some embodiments, R^{y} is —C(O)H.

In some embodiment, R^{w}, R^{x}, and R^{y} are as depicted in the compounds of Table 1B, below.

As described above and defined herein, each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{A} is an optionally substituted C₁₋₆ aliphatic. In some embodiments, R^{A} is an optionally substituted phenyl. In some embodiments, R^{A} is an optionally substituted 3-7 membered saturated or partially unsaturated carbocyclic. In some embodiments, R^{A} is an optionally substituted 3-7 membered saturated or partially unsaturated heterocyclic ring having 1-2 heteroatoms independently selected from nitrogen, oxygen, and sulfur. In some embodiments, R^{A} is an optionally substituted 5-6 membered heteroaryl ring having 1-4 heteroatoms independently selected from nitrogen, oxygen, and sulfur.

In some embodiments, R^{A} is C₁₋₆ alkyl (e.g., methyl, ethyl, isopropyl). In some embodiments, R^{A} is C₁₋₆ haloalkyl (e.g., —CF₃, —CHF₂).

In some embodiment, R^{A} is as depicted in the compounds of Table 1B, below.

As described above and defined herein, each R^{B} is independently -L^{B}-Cy^{B1}-H or -L^{B}-Cy^{B1}-Cy^{B2}.

In some embodiments, R^{B} is -L^{B}-Cy^{B1}-H. In some embodiments, R^{B} is -L^{B}-Cy^{B1}-Cy^{B2}.

In some embodiments, R^{B} is

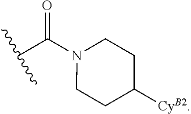

In some embodiments, R^{B} is

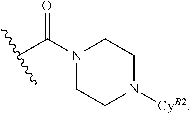

In some embodiments, R^{B} is

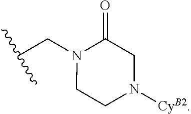

In some embodiments, R^{B} is

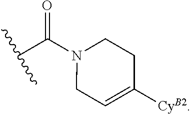

In some embodiments, R^{B} is

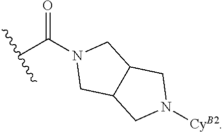

In some embodiments, R^{B} is

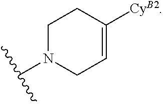

In some embodiments, R^{B} is

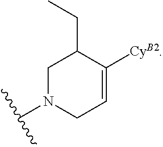

In some embodiments, R^{B} is,

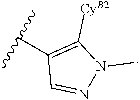

In some embodiments, R^{B} is

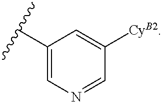

In some embodiments, R^{B} is

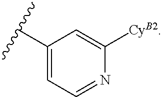

In some embodiments, R^{B} is

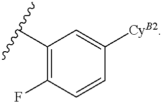

In some embodiments, R^{B} is

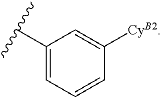

In some embodiments, R^{B} is

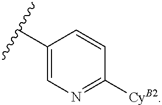

In some embodiments, R^{B} is

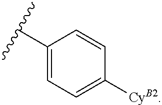

In some embodiments, R^{B} is as depicted in the compounds of Table 1B, below.

In some embodiments, R^{B} is

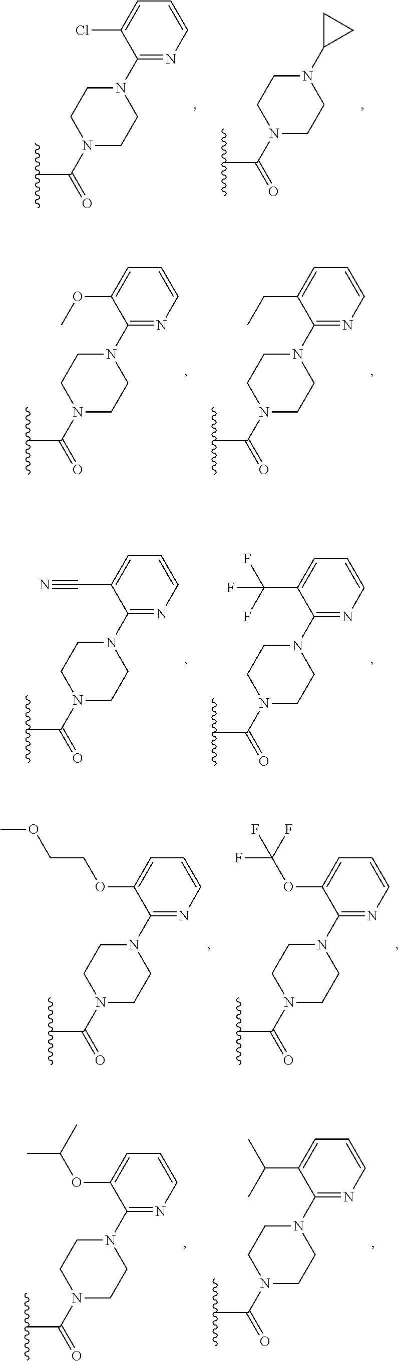

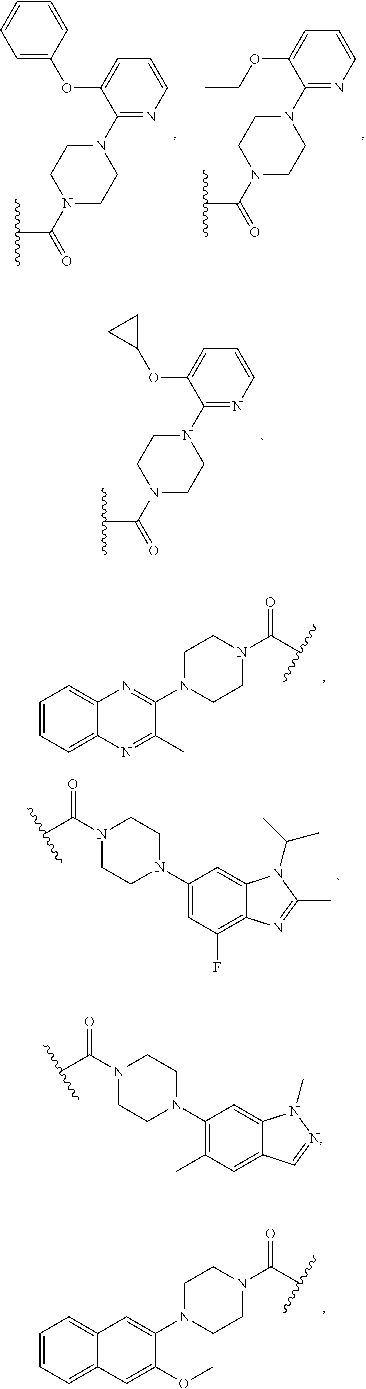

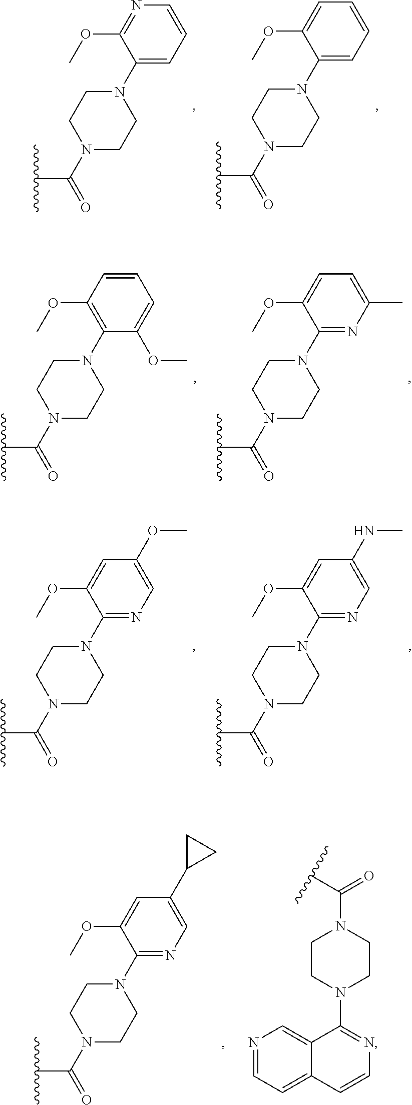

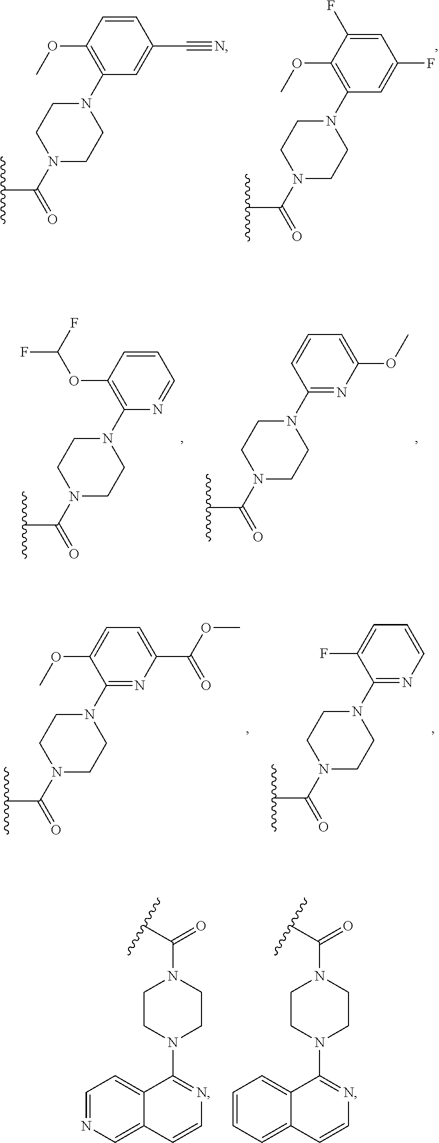

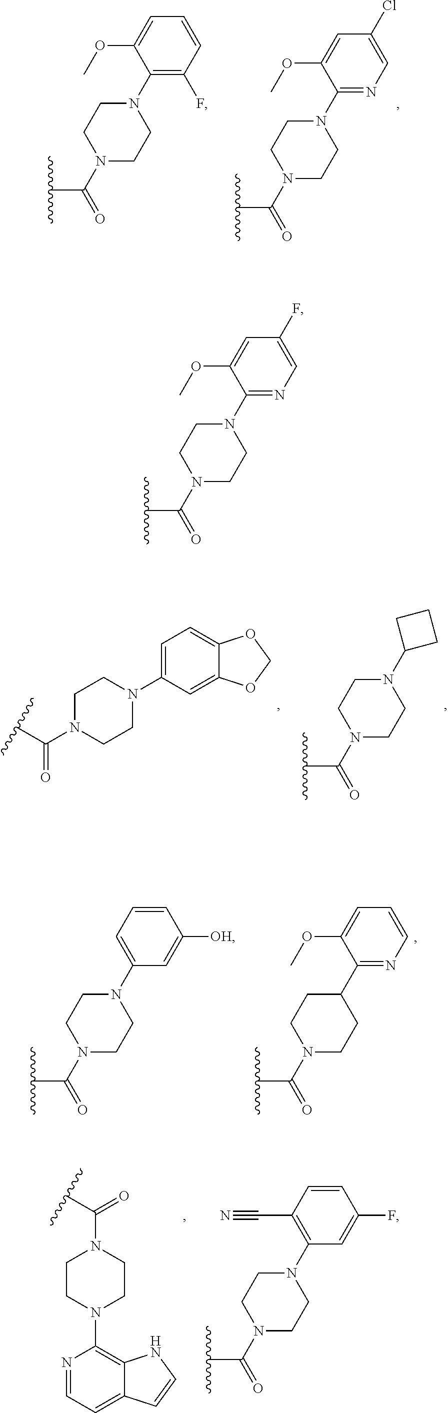

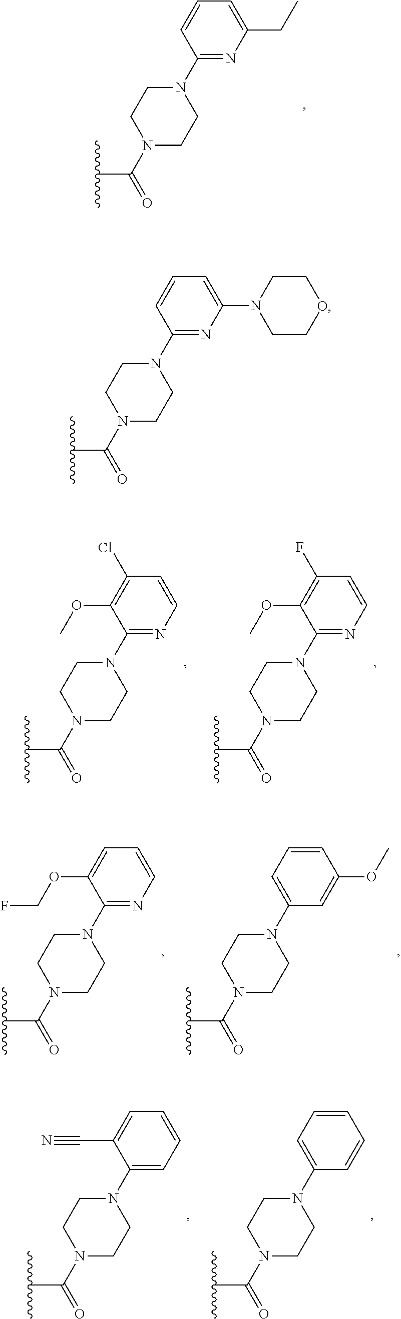

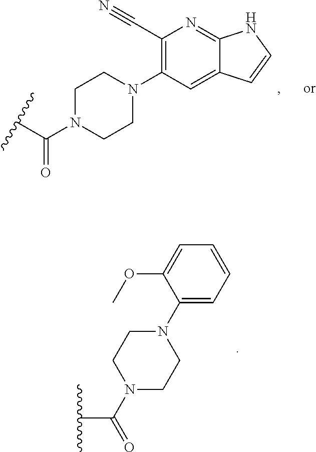 [SMILES: *C(=O)N1CCN(c2cc3cc[nH]c3nc2C#N)CC1 ; *C(=O)N1CCN(c2ccccc2OC)CC1]

In some embodiments, R^{B} is

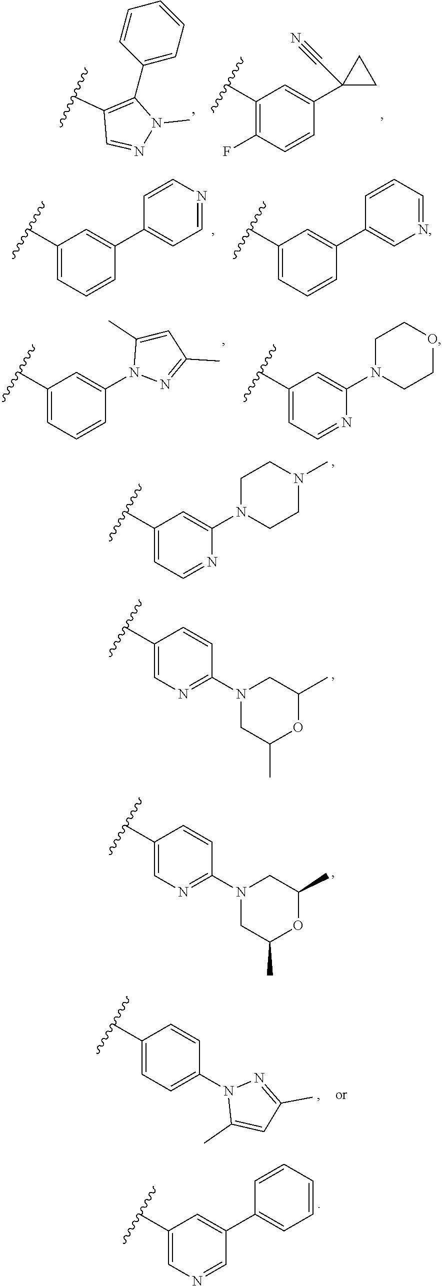

In some embodiments, R^{B} is

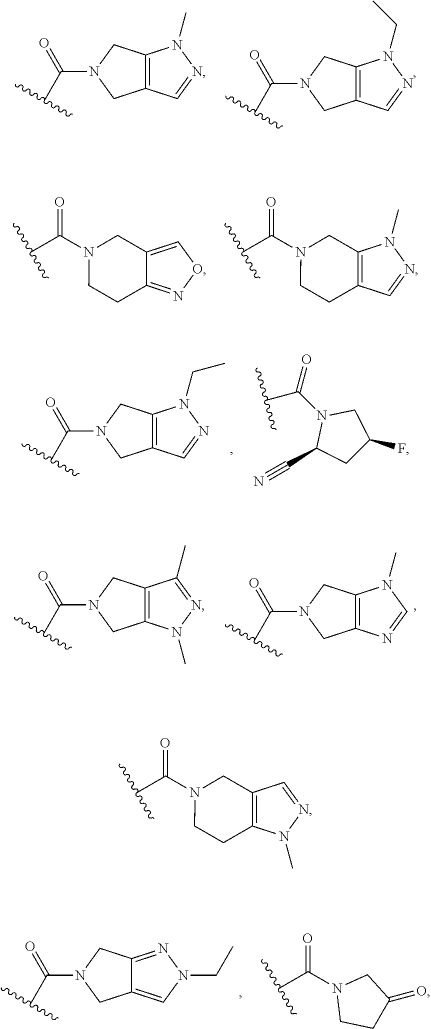

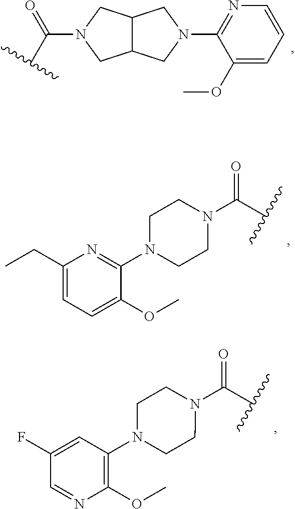 [SMILES: *C(=O)N1CC2CN(c3ncccc3OC)CC2C1 ; *C(=O)N1CCN(c2nc(CC)ccc2OC)CC1 ; *C(=O)N1CCN(c2cc(F)cnc2OC)CC1]

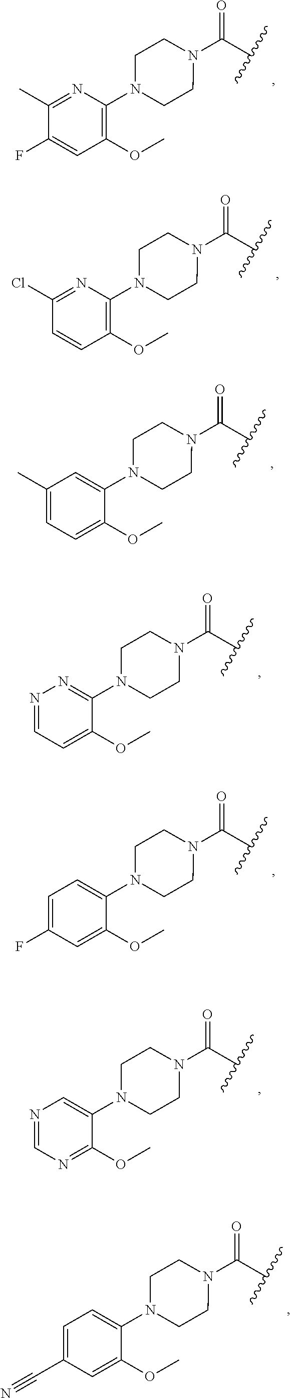

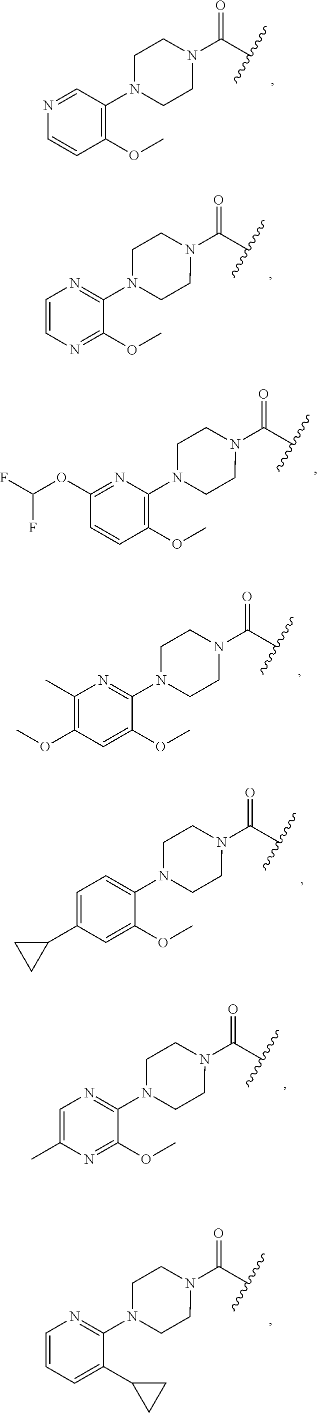

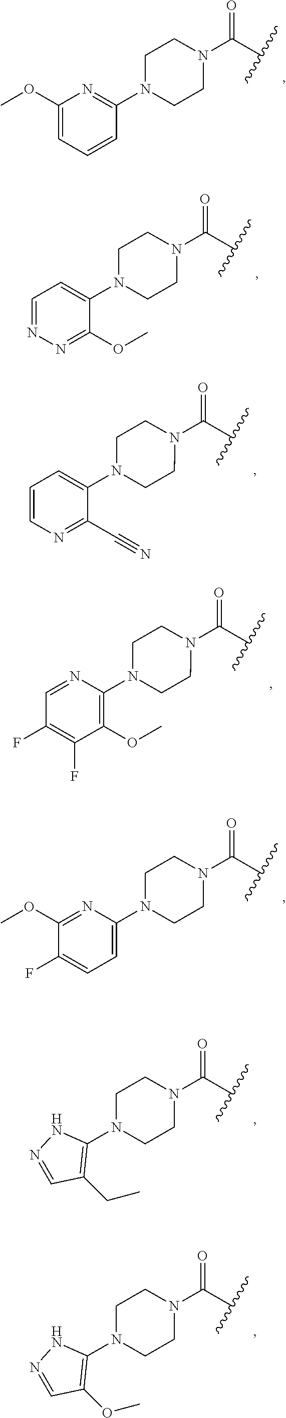

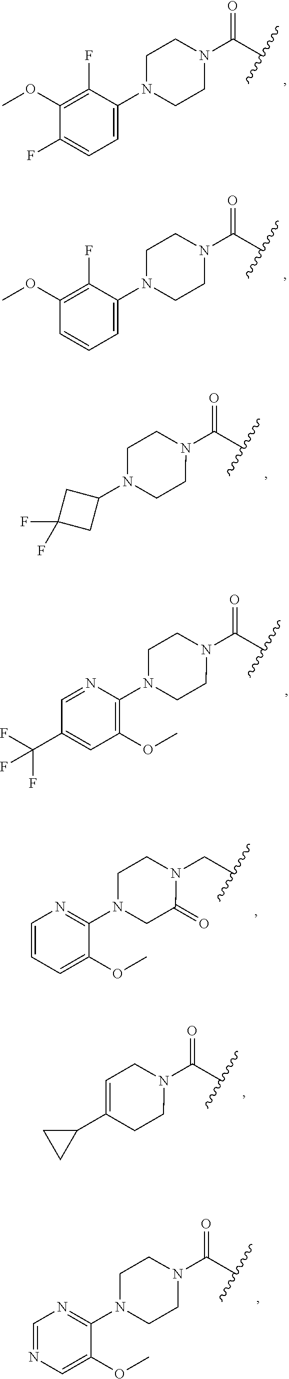

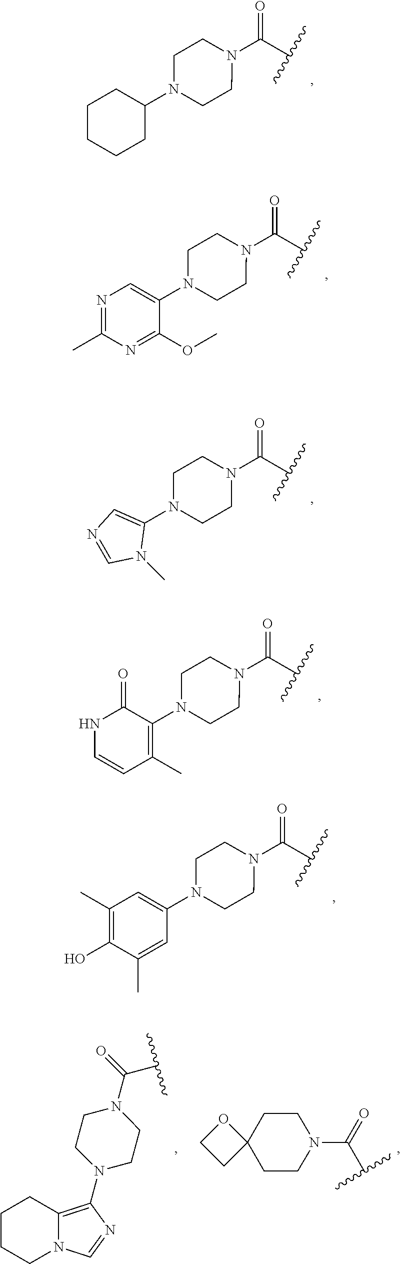

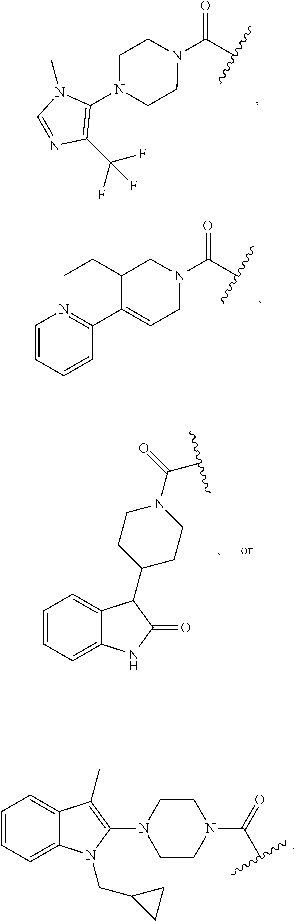 [SMILES: *C(=O)N1CCN(c2c(C(F)(F)F)ncn2C)CC1 ; *C(=O)N1CC=C(c2ccccn2)C(CC)C1 ; *C(=O)N1CCC(C2C(=O)Nc3ccccc32)CC1 ; *C(=O)N1CCN(c2c(C)c3ccccc3n2CC2CC2)CC1]

As described above and defined herein, each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—.

In some embodiments, each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂— —S(O)(NR)— or —CR═CR—.

In some embodiments, L^{B} is a covalent bond. In some embodiments, L^{B} is a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂— —S(O)(NR)— or —CR═CR—. In some embodiments, L^{B} is a C₁₋₃ bivalent straight or branched saturated or unsaturated hydrocarbon chain wherein 1-2 methylene units of the chain are independently and optionally replaced with —O—, —C(O)—, —NR—, —S—, —S(O)—, or —S(O)₂—. In some embodiments, L^{B} is a C₁₋₃ bivalent straight or branched saturated or unsaturated hydrocarbon chain wherein 1-2 methylene units of the chain are independently and optionally replaced with —O—, —C(O)—, or —NR—. In some embodiments, L^{B} is a C₁₋₃ bivalent straight or branched saturated or unsaturated hydrocarbon chain wherein 1 methylene unit of the chain is optionally replaced with —O—, —C(O)—, —NR—, —S—, —S(O)—, or —S(O)₂—. In some embodiments, L^{B} is a C₁₋₃ bivalent straight or branched saturated or unsaturated hydrocarbon chain wherein 1 methylene unit of the chain is optionally replaced with —O—, —C(O)—, or —NR—. In some embodiments, L^{B} is a C₁₋₃ bivalent straight or branched saturated or unsaturated hydrocarbon chain wherein 1 methylene unit of the chain is replaced with —C(O)—. In some embodiments, L^{B} is —C(O)—.

In some embodiments, L^{B} is a C₁₋₃ bivalent straight or branched saturated or unsaturated hydrocarbon chain. In some embodiments, L^{B} is —CH₂—.

In some embodiments, L^{B} is —C(O)—, —C(S)—, —C(NR)R—, —S(O)—, —S(O)₂— or —S(O)(NR)—. In some embodiments, L^{B} is —C(O)—, —C(S)—, or —C(NR)R—. In some embodiments, L^{B} is —C(O)—, —S(O)—, —S(O)₂— or —S(O)(NR)—. In some embodiments, L^{B} is —C(S)—. In some embodiments, L^{B} is —C(NR)R—. In some embodiments, L^{B} is —S(O)—. In some embodiments, L^{B} is —S(O)₂—. In some embodiments, L^{B} is —S(O)(NR)—.

As described above and defined herein, each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, a 5-10 membered monocyclic or bicyclic arylenyl or heteroarylenyl having 1-4 heteroatoms independently selected from nitrogen, oxygen, and sulfur; or a 5-15 membered saturated or partially saturated monocyclic, bicyclic, or tricyclic carbocyclylenyl, heterocyclylenyl, arylenyl, or heteroarylenyl having 1-4 heteroatoms independently selected from nitrogen, oxygen, and sulfur.

In some embodiments, each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur.

It will be appreciated that all embodiments to Cy^{B1} may refer to a terminal ring (or otherwise optionally substituted ring) in structures with -L^{B}-Cy^{B1} (i.e., when R^{B} is -L^{B}-Cy^{B1}-H), or a ring further connected to Cy^{B2} as in structures -L^{B}-Cy^{B1}-Cy^{B2}, regardless of how presented. By way of example, an embodiment to Cy^{B1} is phenylenyl, refers to phenylenyl in -L^{B}-Cy^{B1}-Cy^{B2}, and phenyl in -L^{B}-Cy^{B1}. Similarly, an embodiment to Cy^{B1} is

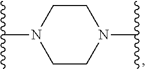 [SMILES: *N1CCN(*)CC1]

refers to

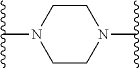 [SMILES: *N1CCN(*)CC1]

in -L^{B}-Cy^{B1}-Cy^{B2}, and

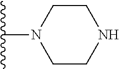 [SMILES: *N1CCNCC1]

in -L^{B}-Cy^{B1}. Similarly, an embodiment to Cy^{B1} is

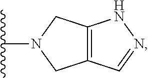 [SMILES: *N1Cc2cn[nH]c2C1]

refers to

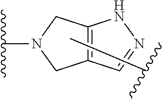

in -L^{B}-Cy^{B1}-Cy^{B2} and

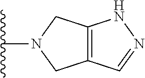 [SMILES: *N1Cc2cn[nH]c2C1]

in -L^{B}-Cy^{B1}.

In some embodiments, each Cy^{B1} is independently an optionally substituted ring selected from phenylenyl, a 3-7 membered saturated or partially unsaturated carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur.

In some embodiments, Cy^{B1} is optionally substituted phenylenyl. In some embodiments, Cy^{B1} is phenylenyl.

In some embodiments, Cy^{B1} is optionally substituted 3-7 membered saturated or partially unsaturated carbocyclylenyl or heterocyclylenyl having 1-4 heteroatoms independently selected from nitrogen, oxygen, and sulfur. In some embodiments, Cy^{B1} is optionally substituted 5-10 membered monocyclic or bicyclic arylenyl or heteroarylenyl having 1-4 heteroatoms independently selected from nitrogen, oxygen, and sulfur.

In some embodiments, Cy^{B1} is optionally substituted 3-7 membered saturated or partially unsaturated heterocyclylenyl having 1-4 heteroatoms independently selected from nitrogen, oxygen, and sulfur. In some embodiments, Cy^{B1} is optionally substituted 6-membered saturated or partially unsaturated heterocyclylenyl having 1-4 heteroatoms independently selected from nitrogen, oxygen, and sulfur. In some embodiments, Cy^{B1} is optionally substituted 6-membered saturated or partially unsaturated heterocyclylenyl having 1-2 heteroatoms independently selected from nitrogen, oxygen, and sulfur. In some embodiments, Cy^{B1} is optionally substituted 6-membered saturated or partially unsaturated heterocyclylenyl having 1-2 nitrogen heteroatoms. In some embodiments, Cy^{B1} is optionally substituted piperadinylenyl or piperazinylenyl.

In some embodiments, Cy^{B1} is a 3-7 membered saturated or partially unsaturated heterocyclylenyl having 1-4 heteroatoms independently selected from nitrogen, oxygen, and sulfur. In some embodiments, Cy^{B1} is a 6-membered saturated or partially unsaturated heterocyclylenyl having 1-4 heteroatoms independently selected from nitrogen, oxygen, and sulfur. In some embodiments, Cy^{B1} is a 6-membered saturated or partially unsaturated heterocyclylenyl having 1-2 heteroatoms independently selected from nitrogen, oxygen, and sulfur. In some embodiments, Cy^{B1} is a 6-membered saturated or partially unsaturated heterocyclylenyl having 1-2 nitrogen heteroatoms. In some embodiments, Cy^{B1} is a piperadinylenyl or piperazinylenyl. In some embodiments, Cy^{B1} is

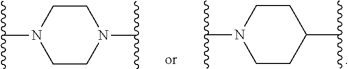 [SMILES: *N1CCN(*)CC1 ; *C1CCN(*)CC1]

In some embodiments, Cy^{B1} is an optionally substituted piperidinonyl or piperazinonyl. In some embodiments, Cy^{B1} is an optionally substituted dihydropyridinyl. In some embodiments, Cy^{B1} is an optionally substituted thiomorpholinyl.

In some embodiments, Cy^{B1} is optionally substituted morpholinyl.

In some embodiments, Cy^{B1} is a 3-10 membered saturated or partially unsaturated monocyclic or bicyclic heterocyclylenyl having 1-4 heteroatoms independently selected from nitrogen, oxygen, and sulfur.

In some embodiments, Cy^{B1} is an optionally substituted 8-10 membered saturated or partially unsaturated bicyclic heterocyclylenyl having 1-4 heteroatoms independently selected from nitrogen, oxygen, and sulfur. In some embodiments, Cy^{B1} is an optionally substituted 8-membered saturated or partially unsaturated heterocyclylenyl having 1-4 heteroatoms independently selected from nitrogen, oxygen, and sulfur. In some embodiments, Cy^{B1} is an optionally substituted 8-membered saturated or partially unsaturated heterocyclylenyl having 1-2 heteroatoms independently selected from nitrogen, oxygen, and sulfur. In some embodiments, Cy^{B1} is an optionally substituted 8-membered saturated or partially unsaturated heterocyclylenyl having 1-2 nitrogen heteroatoms. In some embodiments, Cy^{B1} is optionally substituted

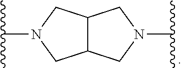 [SMILES: *N1CC2CN(*)CC2C1]

In some embodiments, Cy^{B1} is an optionally substituted

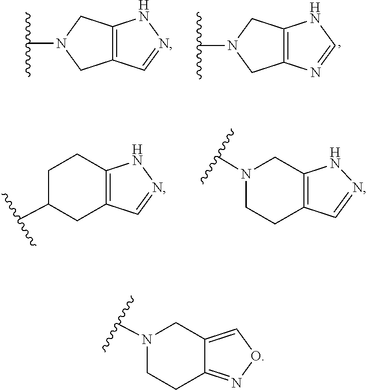

In some embodiments, Cy^{B1} is optionally substituted indazolyl. In some embodiments, Cy^{B1} is optionally substituted pyrrolo[2,3-b]pyridine.

In some embodiments, Cy^{B1} is an optionally substituted 5-6 membered monocyclic heteroarylenyl having 1-4 heteroatoms independently selected from nitrogen, oxygen, and sulfur. In some embodiments, Cy^{B1} is an optionally substituted 6 membered monocyclic heteroarylenyl having 1-4 heteroatoms independently selected from nitrogen, oxygen, and sulfur. In some embodiments, Cy^{B1} is an optionally substituted pyridinonyl, pyrazinonyl, or pyrimidinoyl.

In some embodiments, Cy^{B1} is optionally substituted furanyl. In some embodiments, Cy^{B1} is optionally substituted isoxazolyl. In some embodiments, Cy^{B1} is optionally substituted pyrrolyl. In some embodiments, Cy^{B1} is optionally substituted pyridinonyl. In some embodiments, Cy^{B1} is optionally substituted pyridazinyl. In some embodiments, Cy^{B1} is an optionally substituted 5-15 membered saturated or partially saturated monocyclic, bicyclic, or tricyclic carbocyclylenyl, heterocyclylenyl, arylenyl, or heteroarylenyl having 1-4 heteroatoms independently selected from nitrogen, oxygen, and sulfur.

In some embodiments, Cy^{B1} is an optionally substituted 10-15 membered saturated or partially saturated tricyclic carbocyclylenyl, heterocyclylenyl, arylenyl, or heteroarylenyl having 1-4 heteroatoms independently selected from nitrogen, oxygen, and sulfur. In some embodiments, Cy^{B1} is an optionally substituted 10-15 membered saturated or partially saturated tricyclic heterocyclylenyl having 1-4 heteroatoms independently selected from nitrogen, oxygen, and sulfur. In some embodiments, Cy^{B1} is an optionally substituted 10-15 membered heteroarylenyl having 1-4 heteroatoms independently selected from nitrogen, oxygen, and sulfur.

In some embodiments, Cy^{B1} is a 13-membered saturated or partially saturated tricyclic carbocyclylenyl, heterocyclylenyl, arylenyl, or heteroarylenyl having 1-4 heteroatoms independently selected from nitrogen, oxygen, and sulfur. In some embodiments, Cy^{B1} is a 15-membered saturated or partially saturated tricyclic carbocyclylenyl, heterocyclylenyl, arylenyl, or heteroarylenyl having 1-4 heteroatoms independently selected from nitrogen, oxygen, and sulfur.

As described above and defined herein, each Cy^{B2} is independently an optionally substituted ring selected from phenyl, a 3-10 membered saturated or partially unsaturated monocyclic or bicyclic carbocyclic or heterocyclic ring having 1-4 heteroatoms independently selected from nitrogen, oxygen, and sulfur, a 5-10 membered monocyclic or bicyclic aryl or heteroaryl having 1-4 heteroatoms independently selected from nitrogen, oxygen, and sulfur; or a 5-15 membered saturated or partially saturated monocyclic, bicyclic, or tricyclic carbocyclyl, heterocyclyl, aryl, or heteroaryl having 1-4 heteroatoms independently selected from nitrogen, oxygen, and sulfur.

In some embodiments, each Cy^{B2} is independently an optionally substituted ring selected from phenyl, a 3-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur.

In some embodiments, each Cy^{B2} is independently an optionally ring selected from phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur.

In some embodiments, Cy^{B2} is optionally substituted phenyl. In some embodiments, Cy^{B2} is phenyl. In some embodiments, Cy^{B2} is phenyl, optionally substituted with —CN, halogen, —R^{◯}, or —OR^{◯}, wherein R^{◯} is hydrogen or C₁₋₆ aliphatic.

In some embodiments, Cy^{B2} is optionally substituted 3-7 membered saturated or partially unsaturated carbocyclic or heterocyclic ring having 1-2 heteroatoms independently selected from nitrogen, oxygen, and sulfur.

In some embodiments, Cy^{B2} is optionally substituted 3-7 membered saturated or partially unsaturated carbocyclic. In some embodiments, Cy^{B2} is optionally substituted cyclopropyl. In some embodiments, Cy^{B2} is cyclopropyl. In some embodiments, C^{y} is optionally substituted cyclobutyl. In some embodiments, Cy^{B2} is cyclobutyl.

In some embodiments, Cy^{B2} is optionally substituted cyclopentyl. In some embodiments, Cy^{B2} is cyclopentyl. In some embodiments, Cy^{B2} is optionally substituted cyclohexyl. In some embodiments, Cy^{B2} is cyclohexyl.

In some embodiments, Cy^{B2} is optionally substituted 5-10 membered monocyclic or bicyclic aryl or heteroaryl with 1-4 heteroatoms independently selected from nitrogen, oxygen and sulfur.

In some embodiments, Cy^{B2} is optionally substituted 5-6 membered monocyclic heteroaryl with 1-4 heteroatoms independently selected from nitrogen, oxygen and sulfur. In some embodiments, Cy^{B2} is optionally substituted 5-6 membered monocyclic heteroaryl with 1-2 heteroatoms independently selected from nitrogen, oxygen and sulfur. In some embodiments, Cy^{B2} is optionally substituted pyridinyl. In some embodiments, Cy^{B2} is pyridinyl, optionally substituted with —CN, halogen, —R^{◯}, —OR^{◯}, —N(R^{◯})₂—C(O)OR^{◯}, wherein each R^{◯} is independently hydrogen; C₁₋₆ aliphatic, which may be optionally substituted with halogen, —(CH₂)₀₋₂OH, or —(CH₂)₀₋₂OR^{●}, where R^{●} is C₁₋₄ aliphatic; or a 5-6-membered saturated, partially unsaturated, or aryl ring having 0-4 heteroatoms independently selected from nitrogen, oxygen, or sulfur (e.g., phenyl or morpholinyl).

In some embodiments, Cy^{B2} is optionally substituted 6 membered monocyclic heteroaryl with 1-2 heteroatoms independently selected from nitrogen, oxygen and sulfur. In some embodiments, Cy^{B2} is optionally substituted pyridinyl, pyrimidinyl, pyridazinyl, pyrazinyl, or triazinyl.

In some embodiments, Cy^{B2} is optionally substituted optionally substituted pyridinonyl, pyrazinonyl, or pyrimidinoyl

In some embodiments, Cy^{B2} is optionally substituted 5 membered monocyclic heteroaryl with 1-2 heteroatoms independently selected from nitrogen, oxygen and sulfur. In some embodiments, Cy^{B2} is optionally substituted pyrazolyl, imidazolyl, or triazolyl.

In some embodiments, Cy^{B2} is optionally substituted 8-10 membered bicyclic aryl or heteroaryl with 1-4 heteroatoms independently selected from nitrogen, oxygen and sulfur.

In some embodiments, Cy^{B2} is optionally substituted 9-membered bicyclic heteroaryl with 1-4 heteroatoms independently selected from nitrogen, oxygen and sulfur. In some embodiments, Cy^{B2} is optionally substituted 9-membered bicyclic heteroaryl with 1-4 nitrogen heteroatoms. In some embodiments, Cy^{B2} is optionally substituted benzimidazolyl, indazolyl, or azaindolyl (e.g., pyrrolo[2,3-c]pyridinyl or pyrrolo[2,3-b]pyridinyl). In some embodiments, Cy^{B2} is benzimidazolyl indazolyl, or azaindolyl (e.g., pyrrolo[2,3-c]pyridinyl or pyrrolo[2,3-b]pyridinyl) optionally substituted with —CN, halogen or —R^{◯}, wherein R^{◯} is hydrogen or C₁₋₆ aliphatic. In some embodiments, Cy^{B2} is optionally substituted indolyl or azaindolyl.

In some embodiments, Cy^{B2} is optionally substituted [1,2,4]triazolo[4,3-a]pyridinyl.

In some embodiments, Cy^{B2} is optionally substituted 10-membered bicyclic aryl or heteroaryl with 1-4 heteroatoms independently selected from nitrogen, oxygen and sulfur. In some embodiments, Cy^{B2} is optionally substituted 10-membered bicyclic heteroaryl with 1-4 nitrogen heteroatoms. In some embodiments, Cy^{B2} is optionally substituted quinoxalinyl, isoquinolinyl, 2,6-naphthyridinyl, or 2,7-naphthyridinyl. In some embodiments, Cy^{B2} is quinoxalinyl, isoquinolinyl, 2,6-naphthyridinyl, or 2,7-naphthyridinyl, optionally substituted with —R^{◯}, wherein R^{◯} is hydrogen or C₁₋₆ aliphatic.

In some embodiments, Cy^{B2} is optionally substituted naphthalenyl. In some embodiments, Cy^{B2} is naphthalenyl, optionally substituted with —R^{◯} or —OR^{◯}, wherein R^{◯} is hydrogen or C₁₋₆ aliphatic.

In some embodiments, Cy^{B2} is optionally substituted benzo[d][1,3]dioxolyl.

In some embodiments Cy^{B2} is

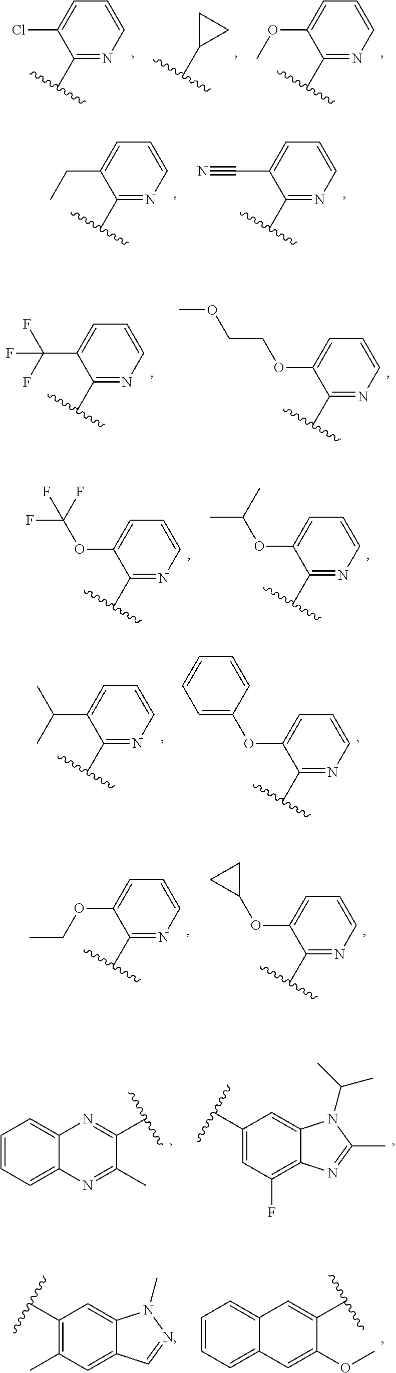

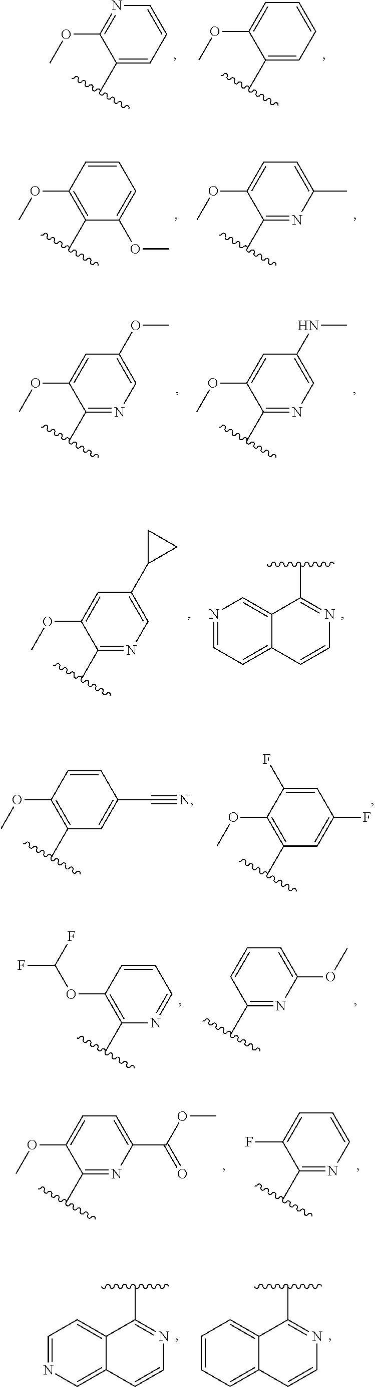

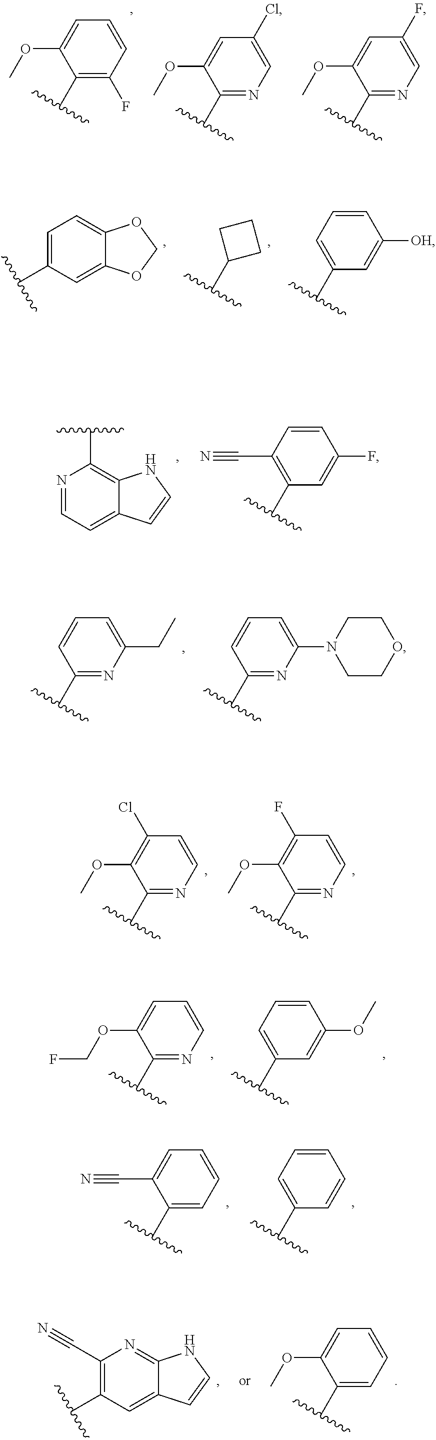

In some embodiments, Cy^{B2} is an optionally substituted 5-15 membered saturated or partially saturated monocyclic, bicyclic, or tricyclic carbocyclyl, heterocyclyl, aryl, or heteroaryl having 1-4 heteroatoms independently selected from nitrogen, oxygen, and sulfur.

In some embodiments, Cy^{B2} is an optionally substituted 10-15 membered saturated or partially saturated tricyclic carbocyclyl, heterocyclyl, aryl, or heteroaryl having 1-4 heteroatoms independently selected from nitrogen, oxygen, and sulfur. In some embodiments, Cy^{B2} is an optionally substituted 10-15 membered saturated or partially saturated tricyclic heterocyclyl having 1-4 heteroatoms independently selected from nitrogen, oxygen, and sulfur. In some embodiments, Cy^{B2} is an optionally substituted 10-15 membered heteroaryl having 1-4 heteroatoms independently selected from nitrogen, oxygen, and sulfur.

In some embodiments, Cy^{B2} is a 13 membered saturated or partially saturated tricyclic carbocyclyl, heterocyclyl, aryl, or heteroaryl having 1-4 heteroatoms independently selected from nitrogen, oxygen, and sulfur. In some embodiments, Cy^{B2} is a 15 membered saturated or partially saturated tricyclic carbocyclyl, heterocyclyl, aryl, or heteroaryl having 1-4 heteroatoms independently selected from nitrogen, oxygen, and sulfur.

As described above and defined herein, each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom or adjacent atoms are optionally taken together with their intervening atoms to form a 3-7 membered saturated or partially unsaturated ring having 0-3 heteroatoms, in addition to the atom or adjacent atoms to which they are attached, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated carbocyclic ring. In some embodiments, R is an optionally substituted 4-7 membered saturated or partially unsaturated heterocyclic ring having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same atom are optionally taken together with their intervening atoms to form a 3-7 membered saturated or partially unsaturated ring having 0-3 heteroatoms, in addition to the atom to which they are attached, independently selected from nitrogen, oxygen, and sulfur. In some embodiments, two R groups on adjacent atoms are optionally taken together with their intervening atoms to form a 3-7 membered saturated or partially unsaturated ring having 0-3 heteroatoms, in addition to the adjacent atoms to which they are attached, independently selected from nitrogen, oxygen, and sulfur.

In some embodiments, R is an optionally substituted 4-7 membered saturated or partially unsaturated carbocyclic ring. In some embodiments, R is an optionally substituted 3 membered saturated or partially unsaturated carbocyclic ring.

In some embodiments, R is as depicted in the compounds of Table 1B, below.

As described above and defined herein, each of w, x, y, and z are independently 0, 1, 2, 3, or 4.

In some embodiments, one or more of w, x, and y is 0. In some embodiments, one or more of w, x, and y is 1. In some embodiments, one or more of w, x, and y is 2. In some embodiments, one or more of w, x, and y is 3. In some embodiments, one or more of w, x, and y is 4.

In some embodiments, w is 0. In some embodiments, w is 1. In some embodiments, w is 2. In some embodiments, w is 3. In some embodiments, w is 4.

In some embodiments, x is 0. In some embodiments, x is 1. In some embodiments, x is 2. In some embodiments, x is 3. In some embodiments, x is 4.

In some embodiments, y is 0. In some embodiments, y is 1. In some embodiments, y is 2. In some embodiments, y is 3. In some embodiments, y is 4.

In some embodiments, z is 0. In some embodiments, z is 1. In some embodiments, z is 2. In some embodiments, z is 3. In some embodiments, z is 4. In some embodiments, z is 1, 2, or 3. In some embodiments, z is 2, 3, or 4.

In some embodiment, w, x, y, and z are as depicted in the compounds of Table 1B, below.

In certain embodiments, the present invention provides a compound of formula I′, wherein the compound is of formula I-a-8:

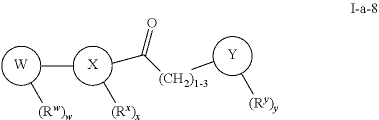

or a pharmaceutically acceptable salt thereof, wherein Ring W, Ring X, Ring Y, R^{w}, R^{x}, R^{y}, w, x, and y are as defined above and described herein both individually and in combination.

In certain embodiments, the present invention provides a compound of formula I′, wherein the compound is of formula I-a-15:

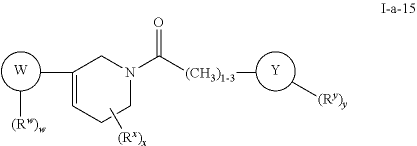

or a pharmaceutically acceptable salt thereof, wherein Ring W, Ring Y, R^{w}, R^{x}, R^{y}, w, x, and y are as defined above and described herein both individually and in combination.

In certain embodiments, the present invention provides a compound of formula I′, wherein the compound is of formula I-a-16:

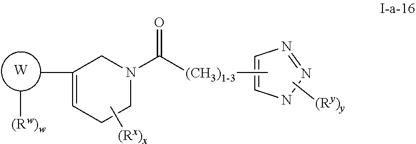

or a pharmaceutically acceptable salt thereof, wherein Ring W, R^{w}, R^{x}, R^{y}, w, x, and y are as defined above and described herein both individually and in combination.

In certain embodiments, the present invention provides a compound of formula I′, wherein the compound is of one of formula I-a-1b or I-a-2b:

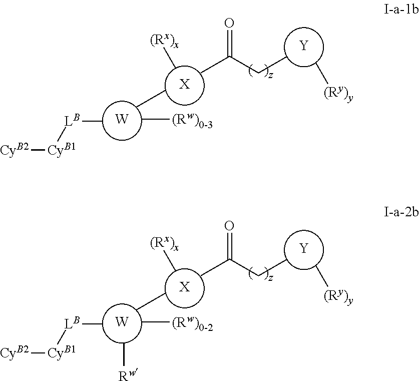

or a pharmaceutically acceptable salt thereof, wherein each variable is as defined above and descried herein both individually and in combination, and wherein:
R^{w′} is R^{w}, wherein R^{w} is as defined above and described herein both individually and in combination.

In some embodiments, R^{w′} is an optionally substituted ring selected from phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{w′} is an optionally substituted 3-7 membered saturated or partially unsaturated carbocyclic ring. In some embodiments, R^{w′} is an optionally substituted 3-7 membered saturated or partially unsaturated carbocyclic ring. In some embodiments, R^{w′} is an optionally substituted cyclopropyl. In some embodiments, R^{w′} is an optionally substituted cyclobutyl. In some embodiments, R is an optionally substituted cyclopentyl. In some embodiments, R^{w′} is an optionally substituted cyclohexyl. In some embodiments, R^{w′} is an optionally substituted cyclopropenyl. In some embodiments, R^{w′} is an optionally substituted cyclobutenyl. In some embodiments, R^{w′} is an optionally substituted cyclopentenyl. In some embodiments, R^{w} is an optionally substituted cyclohexenyl.

In some embodiments, R^{w′} is an optionally substituted 3-7 membered saturated or partially unsaturated heterocyclic ring having 1-2 heteroatoms independently selected from nitrogen, oxygen, and sulfur. In some embodiments, R^{w′} is an optionally substituted 5-6 membered saturated or partially unsaturated heterocyclic ring having 1-2 heteroatoms independently selected from nitrogen, oxygen, and sulfur.

In some embodiments, R^{w′} is an optionally substituted 4-membered saturated or partially unsaturated heterocyclic ring having 1-2 heteroatoms independently selected from nitrogen, oxygen, and sulfur. In some embodiments, R^{w′} is an optionally substituted azetidinyl, oxetanyl, or thietanyl.

In some embodiments, R^{w′} is an optionally substituted 5-membered saturated or partially unsaturated heterocyclic ring having 1-2 heteroatoms independently selected from nitrogen, oxygen, and sulfur. In some embodiments, R^{w′} is an optionally substituted pyrrolidinyl, pyrrolinyl, pyrazolidinyl, pyrazolinyl, imidazolidinyl, or imidazolinyl. In some embodiments, R^{w′} is an optionally substituted dihydropyridinyl, pyrrolidinyl, dihydrofuranyl, tetrahydrofuranyl, dihydrothiophenyl, or tetrahydrothiophenyl.

In some embodiments, R^{w′} is an optionally substituted 6-membered saturated or partially unsaturated heterocyclic ring having 1-2 heteroatoms independently selected from nitrogen, oxygen, and sulfur. In some embodiments, R^{w′} is an optionally substituted piperidinyl, piperazinyl, tetrahydropyranyl, 2H-pyranyl, 4H-pyranyl, 1,4-dioxanyl, 1,4-dioxinyl, thianyl, 2H-thiopyranyl, 4H-thiopyranyl, 1,3-dithanyl, 1,4-dithanyl, morpholinyl, or thiomorpholinyl. In some embodiments, R^{w′} is an optionally substituted dihydropyridinyl, tetrahydropyridinyl, dihydropyranyl, tetrahydropyranyl, dihydrothiopyranyl, or tetrahydrothiopyranyl.

In some embodiments, R^{w′} is an optionally substituted 5-6 membered heteroaryl having 1-4 heteroatoms independently selected from nitrogen, oxygen, and sulfur. In some embodiments, R^{w′} is an optionally substituted 5-membered heteroaryl having 1-4 heteroatoms independently selected from nitrogen, oxygen, and sulfur. In some embodiments, R^{w′} is an optionally substituted pyrazolyl, imidazolyl, triazolyl, or tetrazolyl. In some embodiments, R^{w′} is an optionally substituted imidazolyl, optionally substituted with —C(O)N(R^{◯})₂. In some embodiments, R^{w′} is an optionally substituted furanyl, thiophenyl, oxazolyl, isoxazolyl, isothiazolyl, thiazolyl, oxadiazolyl, or thiadiazolyl. In some embodiments, R^{w′} is furanyl, optionally substituted with —C(O)N(R^{◯})₂.

In some embodiments, R^{w′} is an optionally substituted 6-membered heteroaryl having 1-4 heteroatoms independently selected from nitrogen, oxygen, and sulfur. In some embodiments, R^{w′} is an optionally substituted 6 membered heteroaryl having 1-4 nitrogen heteroatoms. In some embodiments, R^{w′} is optionally substituted pyridinyl, pyrimidinyl, pyridazinyl, or triazinyl. In some embodiments, R^{w′} is an optionally substituted pyridinonyl, pyrazinonyl, or pyrimidinoyl.

In some embodiments, R^{w′} is an optionally substituted 7-11 membered bicyclic heterocyclic ring having 1-4 heteroatoms independently selected from nitrogen, oxygen, and sulfur. In some embodiments, R^{w′} is an optionally substituted 9-10 membered bicyclic heterocyclic ring having 1-4 heteroatoms independently selected from nitrogen, oxygen, and sulfur. In some embodiments, R^{w′} is an optionally substituted benzo[d][1,3]dioxolyl. In some embodiments, R^{w′} is an optionally substituted an 11-membered bicyclic heterocyclic ring having 1-4 heteroatoms independently selected from nitrogen, oxygen, and sulfur.

In some embodiments, R^{w′} is an optionally substituted 8-11 membered bicyclic aryl or heteroaryl having 1-4 heteroatoms independently selected from nitrogen, oxygen and sulfur. In some embodiments, R^{w′} is an optionally substituted naphthalenyl. In some embodiments, R^{w′} is an optionally substituted dihydrobenzodioxepinyl. In some embodiments, R^{w′} is an optionally substituted indenyl or dihydroindenyl.

In some embodiments, R^{w′} is an optionally substituted 9-10 membered bicyclic heteroaryl having 1-4 heteroatoms independently selected from nitrogen, oxygen and sulfur.

In some embodiments, R^{w′} is an optionally substituted 9-membered bicyclic heteroaryl having 1-4 heteroatoms independently selected from nitrogen, oxygen and sulfur. In some embodiments, R^{w′} is an optionally substituted indolyl, benzofuranyl, benzothiophenyl, benzimidazolyl, benzoxazolyl, thienopyridinyl, pyrrolo[3,2-b]pyridinyl, pyrrolo[2,3,-b]pyridinyl, pyrazolyl[1,5-a]pyridinyl, or imidazo[1,2-a]pyridinyl, azaindazolyl (e.g., 4-, 5-, 6-, or 7-azaindazolyl), pyrrolol[2,3-c]pyridinyl or indolizinyl.

In some embodiments, R^{w′} is an optionally substituted 10-membered bicyclic heteroaryl having 1-4 heteroatoms independently selected from nitrogen, oxygen and sulfur. In some embodiments, R^{w′} is an optionally substituted quinolinyl, isoquinolinyl, quinolizinyl, quinoxalinyl, phthalazinyl, quinazolinyl, cinnolinyl, or 1,8-naphthyridinyl.

In some embodiments, R^{w′} is

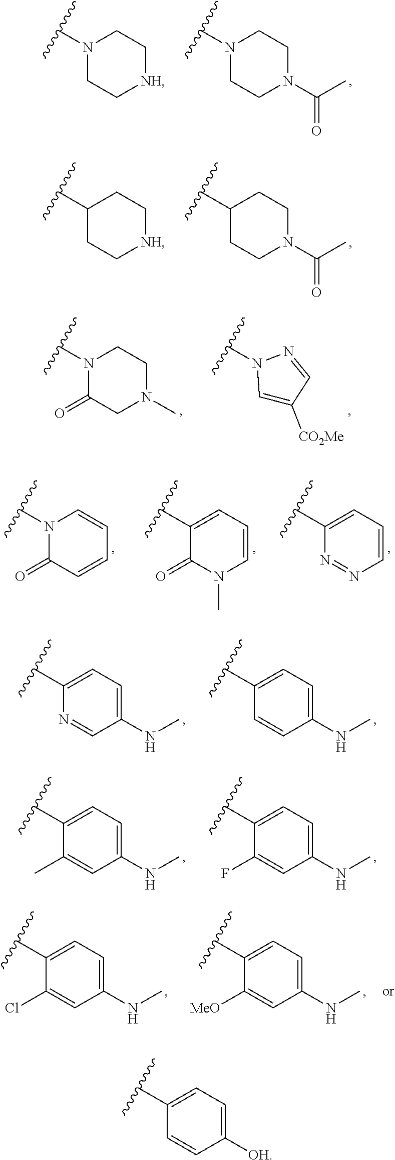

In some embodiments, R^{w′} is

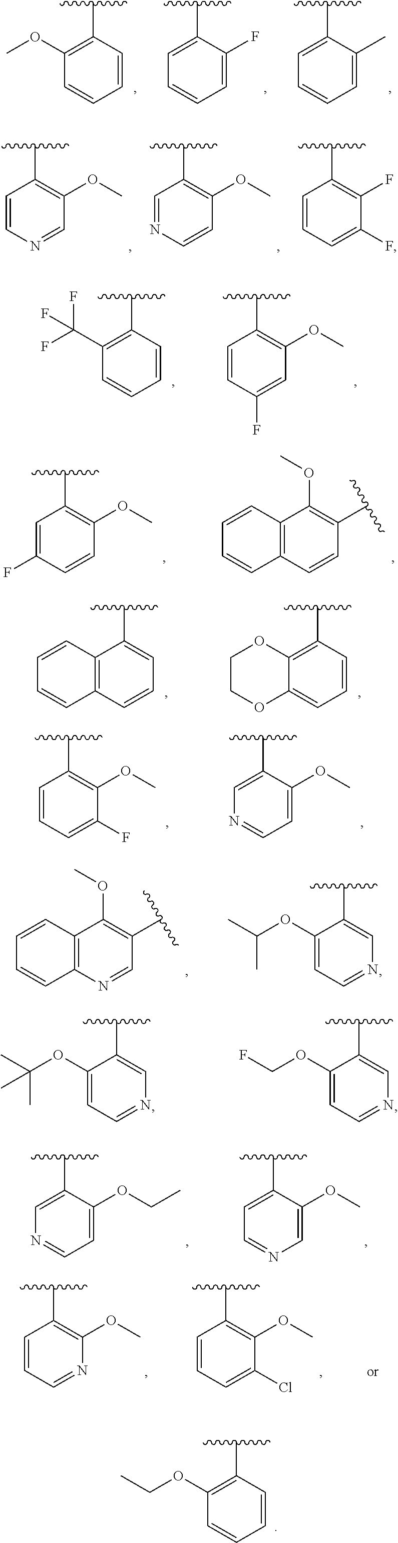

In some embodiments, R^{w′} is

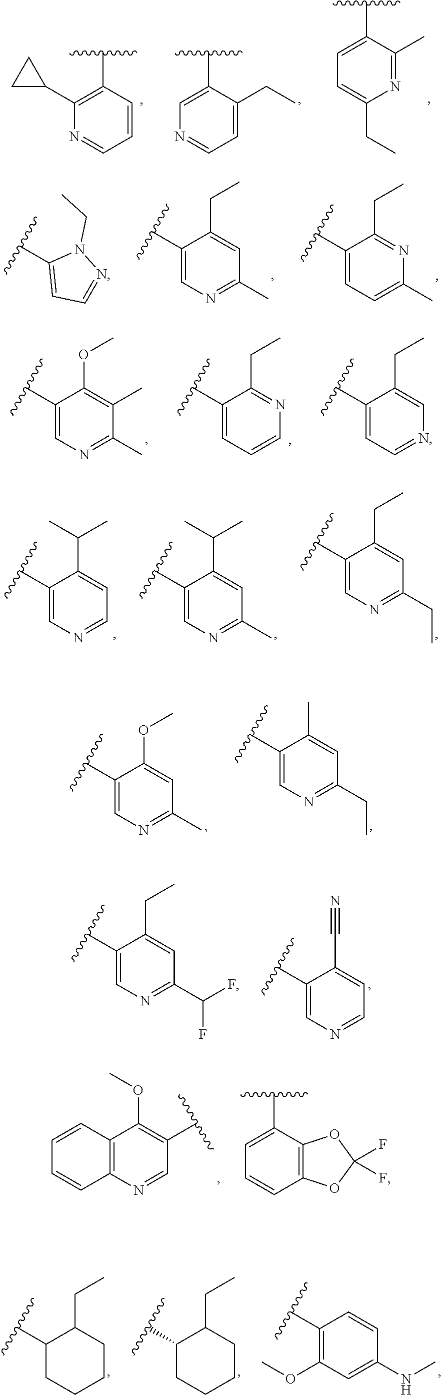

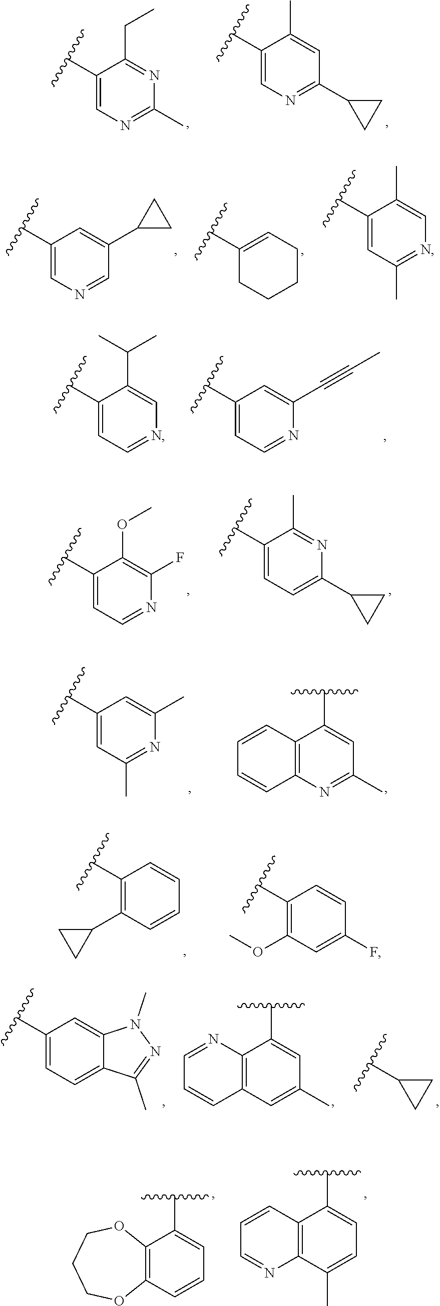

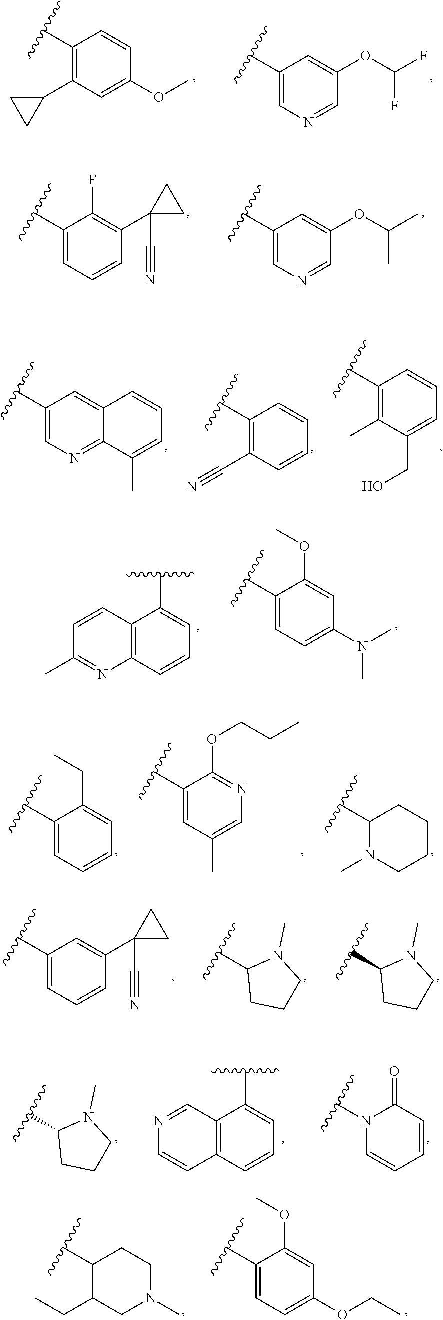

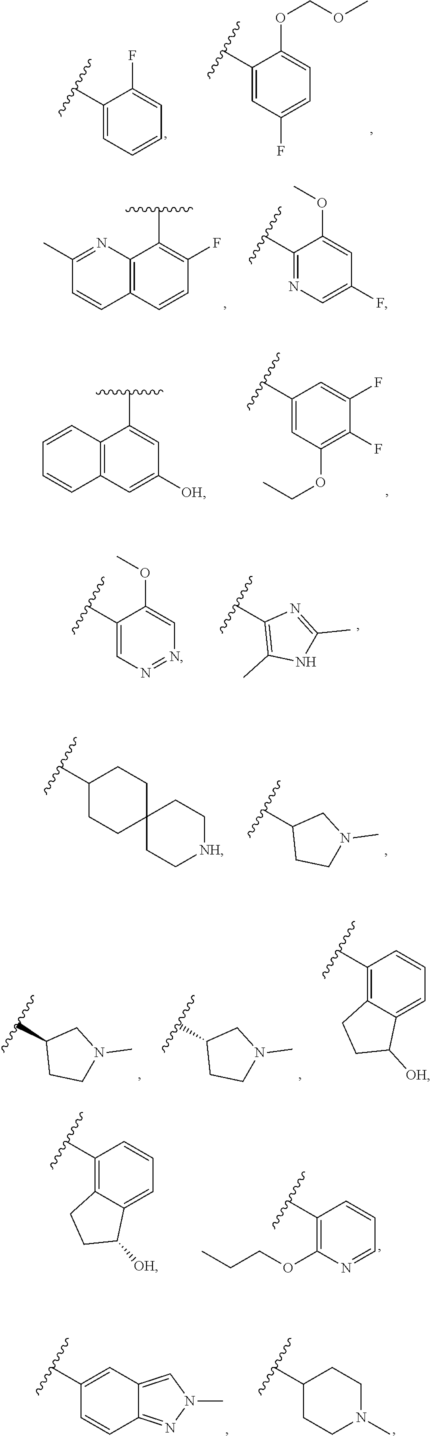

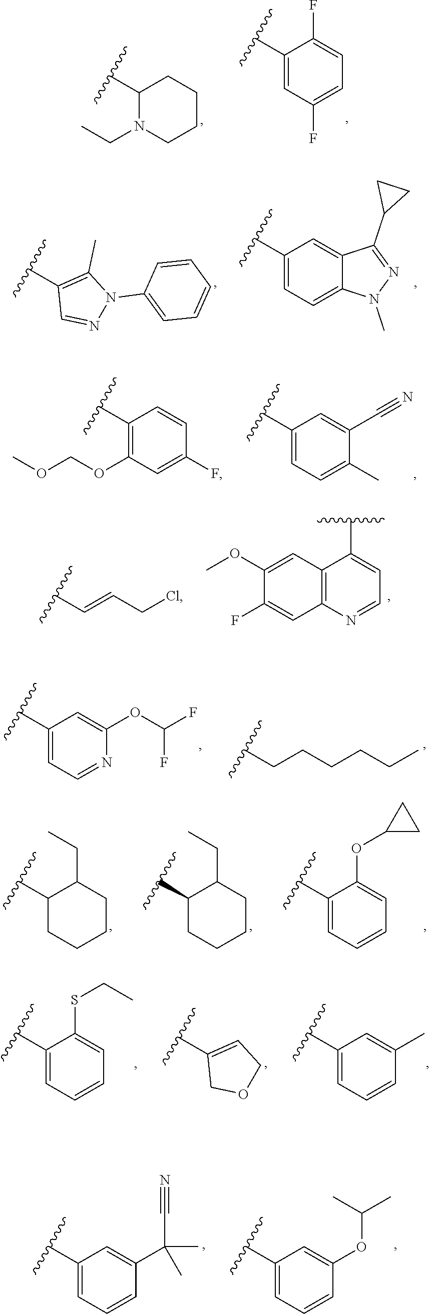

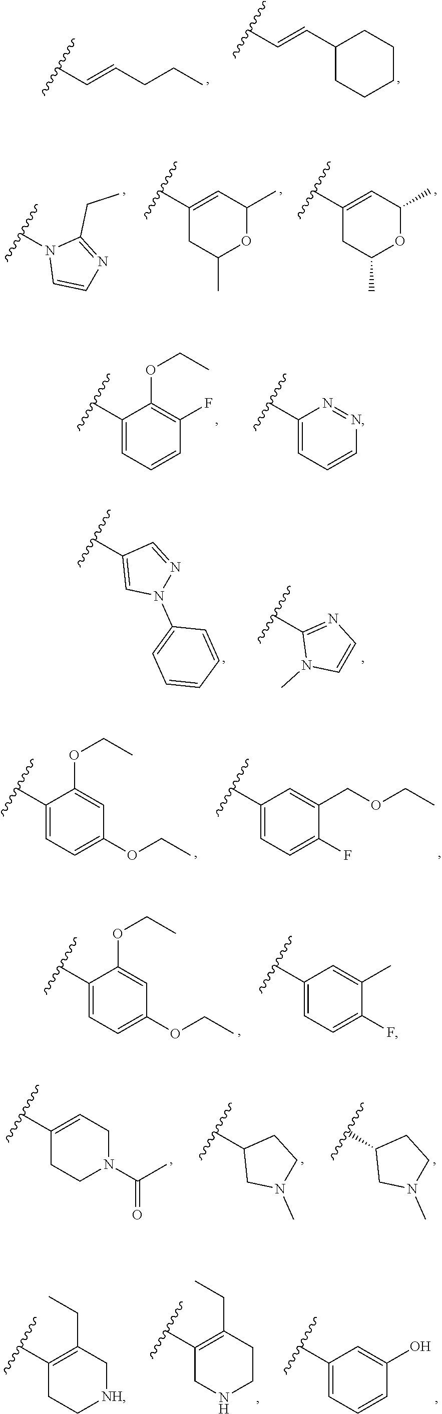

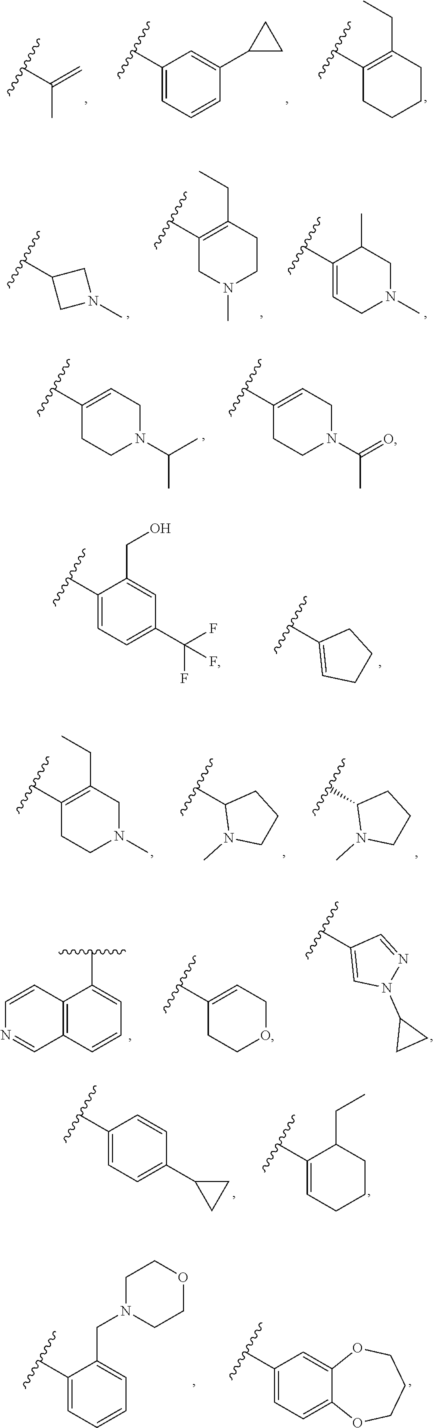

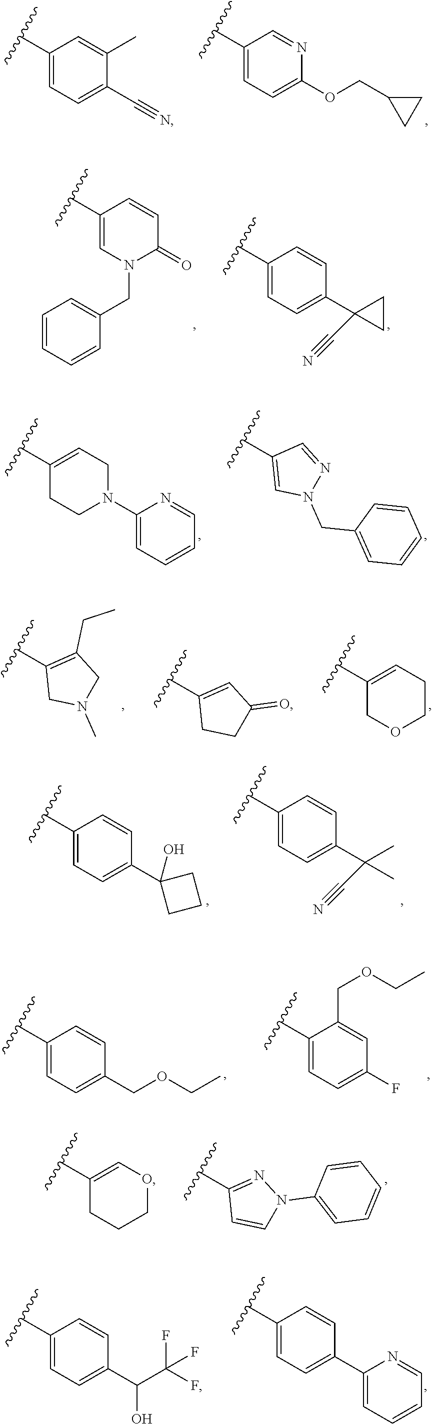

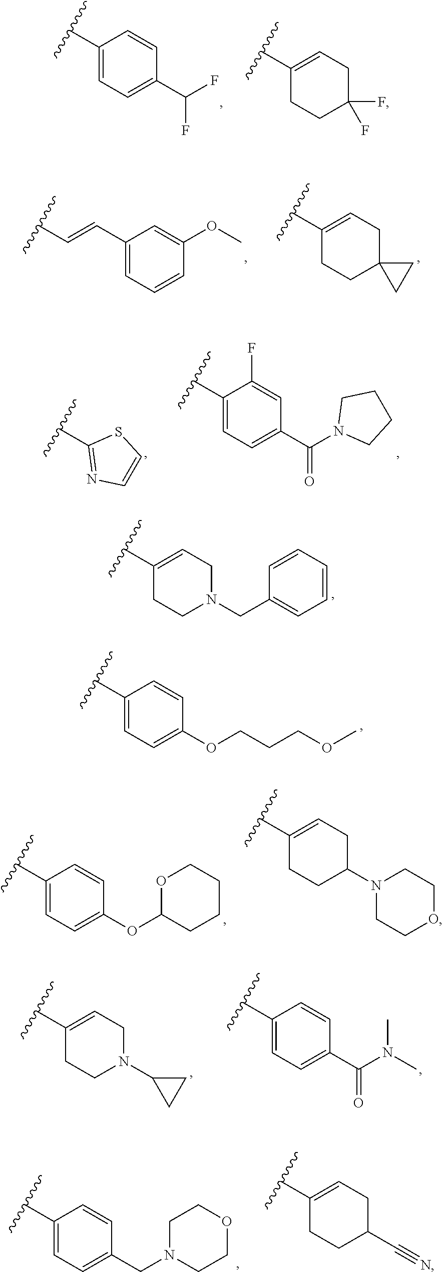

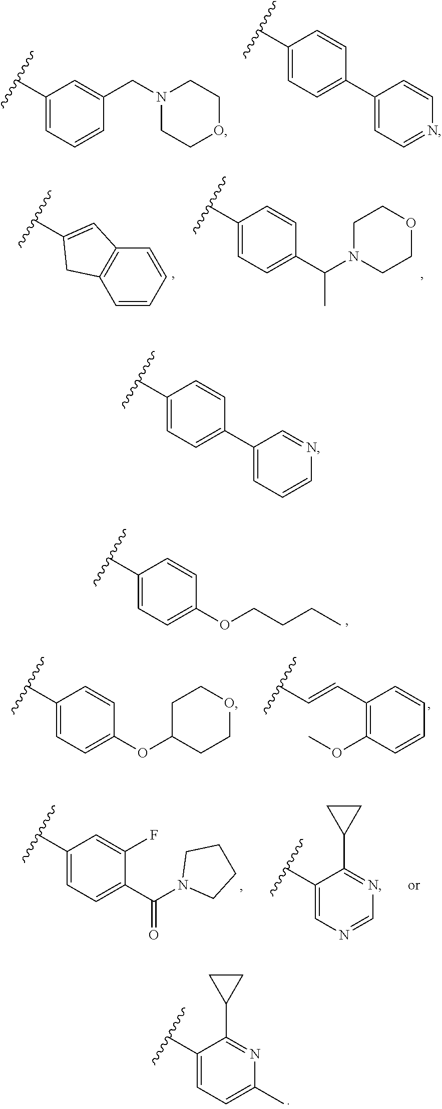

In some embodiments, R^{w′} is an optionally substituted phenyl. In some embodiments, R^{w′} is phenyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is phenyl, optionally substituted with halogen (e.g., fluoro or chloro) or C₁₋₆ aliphatic.

In some embodiments, R^{w′} is an optionally substituted 5 membered saturated or partially unsaturated heterocyclyl having 1-2 heteroatoms independently selected from nitrogen, oxygen, and sulfur. In some embodiments, R^{w′} is an optionally substituted pyrrolidinyl. In some embodiments, R^{w′} is pyrrolidinyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is pyrrolidinyl, optionally substituted with halogen (e.g., fluoro or chloro) or C₁₋₆ aliphatic. In some embodiments, R^{w′} is an optionally substituted 6 membered saturated or partially unsaturated heterocyclyl having 1-2 heteroatoms independently selected from nitrogen, oxygen, and sulfur. In some embodiments, R^{w′} is an optionally substituted piperadinyl. In some embodiments, R^{w′} is piperadinyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is piperadinyl, optionally substituted with halogen (e.g., fluoro or chloro) or C₁₋₆ aliphatic. In some embodiments, R^{w′} is an optionally substituted piperazinyl. In some embodiments, R^{w′} is piperazinyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is piperazinyl, optionally substituted with halogen (e.g., fluoro or chloro) or C₁₋₆ aliphatic.

In some embodiments, R^{w′} is an optionally substituted 5 membered heteroaryl having 1-4 heteroatoms independently selected from nitrogen, oxygen. In some embodiments, R^{w′} is an optionally substituted pyrrolyl, pyrazolyl, imidazolyl, or triazolyl. In some embodiments, R^{w′} is pyrrolyl, pyrazolyl, imidazolyl, or triazolyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is pyrrolyl, pyrazolyl, imidazolyl, or triazolyl, optionally substituted with halogen (e.g., fluoro or chloro) or C₁₋₆ aliphatic. In some embodiments, R^{w′} is an optionally substituted pyrazolyl. In some embodiments, R^{w′} is pyrazolyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is pyrazolyl, optionally substituted with halogen (e.g., fluoro or chloro) or C₁₋₆ aliphatic. In some embodiments, R^{w′} is an optionally substituted imidazolyl. In some embodiments, R^{w′} is imidazolyl, optionally substituted with halogen (e.g., fluoro or chloro), —CN, C₁₋₆ aliphatic optionally further substituted with halogen (e.g., fluoro or chloro), or —OR^{◯}, wherein R^{◯} is hydrogen or C₁₋₆ aliphatic. In some embodiments, R^{w′} is imidazolyl, optionally substituted with halogen (e.g., fluoro or chloro) or C₁₋₆ aliphatic.

In some embodiments, R^{w′} is an optionally substituted 6 membered heteroaryl having 1-4 heteroatoms independently selected from nitrogen, oxygen. In some embodiments, R^{w′} is an optionally substituted pyridinyl, pyridazinyl, pyrimidinyl, pyrazinyl, or triazinyl. In some embodiments, R^{w′} is pyridinyl, pyridazinyl, pyrimidinyl, pyrazinyl, or triazinyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is pyridinyl, pyridazinyl, pyrimidinyl, pyrazinyl, or triazinyl, optionally substituted with halogen (e.g., fluoro or chloro) or C₁₋₆ aliphatic. In some embodiments, R^{w′} is an optionally substituted pyridinyl. In some embodiments, R^{w′} is pyridinyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is pyridinyl, optionally substituted with halogen (e.g., fluoro or chloro) or C₁₋₆ aliphatic. In some embodiments, R^{w′} is an optionally substituted pyridazinyl. In some embodiments, R is pyridazinyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is pyridazinyl, optionally substituted with halogen (e.g., fluoro or chloro) or C₁₋₆ aliphatic. In some embodiments, R^{w′} is an optionally substituted pyridinonyl, pyridazinonyl, pyrimidinonyl, pyrazinonyl, or triazinonyl. In some embodiments, R^{w′} is pyridinonyl, pyridazinonyl, pyrimidinonyl, pyrazinonyl, or triazinonyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is pyridinonyl, pyridazinonyl, pyrimidinonyl, pyrazinonyl, or triazinonyl, optionally substituted with halogen (e.g., fluoro or chloro) or C₁₋₆ aliphatic. In some embodiments, R^{w′} is an optionally substituted pyridinonyl. In some embodiments, R is pyridinonyl, optionally substituted with halogen (e.g., fluoro or chloro), —CN, C₁₋₆ aliphatic optionally further substituted with halogen (e.g., fluoro or chloro), —S(O)₂R^{◯}, or —OR^{◯}, wherein R^{◯} is hydrogen or C₁₋₆ aliphatic. In some embodiments, R^{w′} is pyridinonyl, optionally substituted with halogen (e.g., fluoro or chloro) or C₁₋₆ aliphatic.

In some embodiments, R^{w′} is an optionally substituted cyclopropyl. In some embodiments, R is an optionally substituted 5 membered saturated or partially unsaturated carbocyclyl. In some embodiments, R^{w′} is an optionally substituted cyclopentanyl or cyclopentenyl. In some embodiments, R^{w′} is an optionally substituted 6 membered saturated or partially unsaturated carbocyclyl. In some embodiments, R^{w′} is an optionally substituted cyclohexanyl or cyclohexenyl. In some embodiments, R^{w′} is an optionally substituted naphthalenyl.

In some embodiments, R^{w′} is an optionally substituted 9 membered bicyclic heteroaryl having 1-5 heteroatoms independently selected from nitrogen, oxygen, and sulfur. In some embodiments, R^{w′} is an optionally substituted indolyl, azaindolyl, isoindolyl, azaisoindolyl, indazolylenyl, azaindazolyl, benzimidazolyl, or azabenzimidazolyl. In some embodiments, R^{w′} is an optionally substituted indolyl. In some embodiments, R^{w′} is an optionally substituted benzothiophenyl, benzofuranyl, isobenzofuranyl, benzoisooxazolyl, benzoisothiazolyl, benzoxazolyl, benzothiazolyl, or benzothiadiazolyl.

In some embodiments, R^{w′} is an optionally substituted 8-11 membered saturated or partially unsaturated spiro heterocyclyl having 1-3 heteroatoms independently selected from nitrogen, oxygen, and sulfur. In some embodiments, R^{w′} is an optionally substituted 9-11 membered saturated or partially unsaturated spiro heterocyclyl having 1-3 heteroatoms independently selected from nitrogen, oxygen, and sulfur. In some embodiments, R^{w′} is an optionally substituted 9 membered saturated or partially unsaturated spiro heterocyclyl having 1-3 heteroatoms independently selected from nitrogen, oxygen, and sulfur. In some embodiments, R^{w′} is an optionally substituted 9 membered saturated or partially unsaturated spiro heterocyclyl having 1-3 nitrogen heteroatoms. In some embodiments, R^{w′} is an optionally substituted 4,6-spiro heterocyclyl having 1-3 heteroatoms independently selected from nitrogen, oxygen, and sulfur.

In some embodiments, R^{w′} is an optionally substituted 10 membered saturated or partially unsaturated spiro heterocyclyl having 1-3 heteroatoms independently selected from nitrogen, oxygen, and sulfur. In some embodiments, R^{w′} is an optionally substituted 10 membered saturated or partially unsaturated spiro heterocyclyl having 1-3 nitrogen heteroatoms. In some embodiments, R^{w′} is an optionally substituted 5,6-spiro heterocyclyl having 1-3 heteroatoms independently selected from nitrogen, oxygen, and sulfur.

In some embodiments, R^{w′} is an optionally substituted 11 membered saturated or partially unsaturated spiro heterocyclyl having 1-3 heteroatoms independently selected from nitrogen, oxygen, and sulfur. In some embodiments, R^{w′} is an optionally substituted 11 membered saturated or partially unsaturated spiro heterocyclyl having 1-3 nitrogen heteroatoms. In some embodiments, R^{w′} is an optionally substituted 6,6-spiro heterocyclyl having 1-3 heteroatoms independently selected from nitrogen, oxygen, and sulfur.

In certain embodiments, the present invention provides a compound of formula I′, wherein the compound is of one of formula I-b-5 or I-b-6:

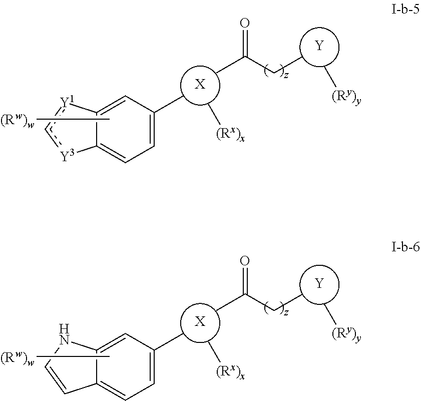

or a pharmaceutically acceptable salt thereof, wherein each variable is as defined above and described herein both individually and in combination.

In certain embodiments, the present invention provides a compound of formula I′, wherein the compound is of one of formula I-c-5 or I-c-6:

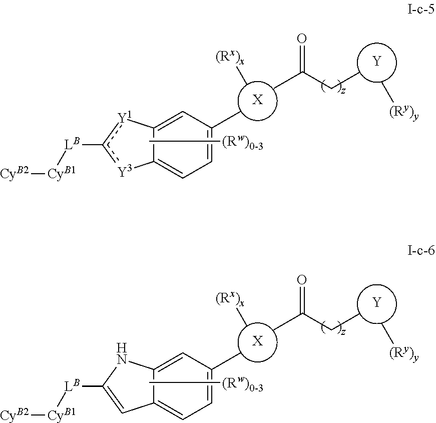

or a pharmaceutically acceptable salt thereof, wherein each variable is as defined above and described herein both individually and in combination.

In certain embodiments, the present invention provides a compound of formula I′, wherein the compound is of one of formula I-d-5 or I-d-6:

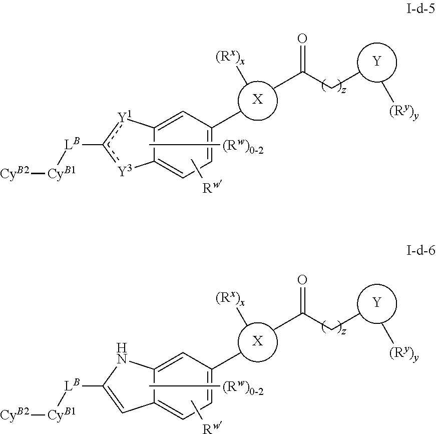

or a pharmaceutically acceptable salt thereof, wherein each variable is as defined above and described herein both individually and in combination.

In certain embodiments, the present invention provides a compound of formula I′, wherein the compound is of one of formula I-e-5 or I-e-6:

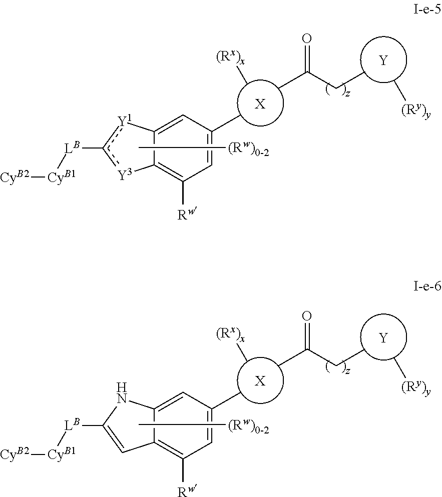

or a pharmaceutically acceptable salt thereof, wherein each variable is as defined above and described herein both individually and in combination.

Exemplary compounds of the invention are set forth in Table 1B and Table 2B, below.

Lengthy table referenced here
US20250214977A1-20250703-T00003
Please refer to the end of the specification for access instructions.

Lengthy table referenced here
US20250214977A1-20250703-T00004
Please refer to the end of the specification for access instructions.

In some embodiments, the present invention provides a compound set forth in Table 1B, above, or a pharmaceutically acceptable salt thereof. In some embodiments, the present invention provides a compound set forth in Table 1B, above.

In some embodiments, the present invention provides a compound set forth in Table 2B, above, or a pharmaceutically acceptable salt thereof. In some embodiments, the present invention provides a compound set forth in Table 2B, above.

In some embodiments, the present invention provides a pharmaceutical composition comprising a compound disclosed herein (described in embodiments herein, both singly and in combination), or a pharmaceutically acceptable salt thereof, together with a pharmaceutically acceptable carrier, adjuvant, or vehicle. For example, in some embodiments, the present invention provides a pharmaceutical composition comprising a compound of formula I′ as defined above, or a pharmaceutically acceptable salt thereof, together with a pharmaceutically acceptable carrier, adjuvant, or vehicle. In some embodiments, the present invention provides a pharmaceutical composition comprising a compound of formula I′ as defined above, together with a pharmaceutically acceptable carrier, adjuvant, or vehicle. In some embodiments, the present invention provides a pharmaceutical composition comprising a compound set forth in Table 1B above, or a pharmaceutically acceptable salt thereof, together with a pharmaceutically acceptable carrier, adjuvant, or vehicle. In some embodiments, the present invention provides a pharmaceutical composition comprising a compound set forth in Table 1B above, together with a pharmaceutically acceptable carrier, adjuvant, or vehicle. In some embodiments, the present invention provides a pharmaceutical composition comprising a compound set forth in Table 2B above, or a pharmaceutically acceptable salt thereof, together with a pharmaceutically acceptable carrier, adjuvant, or vehicle. In some embodiments, the present invention provides a pharmaceutical composition comprising a compound set forth in Table 2B above, together with a pharmaceutically acceptable carrier, adjuvant, or vehicle.

In some embodiments, the present invention provides a compound described herein (such as a compound of formula I′ as defined above), or a pharmaceutically acceptable salt thereof, or a pharmaceutical composition comprising a compound described herein (such as a compound of formula I′ as defined above), or a pharmaceutically acceptable salt thereof, and a pharmaceutically acceptable carrier, adjuvant, or vehicle for use as a medicament.

In some embodiments, the invention also provides a compound described herein (such as a compound of formula I′), or pharmaceutical compositions described herein, for use in a method for modulating (e.g., inhibiting) STAT6 as described herein. In some embodiments, the invention also provides a compound described herein (such as a compound of formula I′), or pharmaceutical compositions described herein, for use in a method for treating a STAT6-mediated disorder as described herein.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

### 5. Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably modulate (e.g., inhibit) STAT6 protein, or a mutant thereof, in a biological sample or in a patient. In certain embodiments, the amount of compound in compositions of this invention is such that is effective to measurably modulate (e.g., inhibit) STAT6 protein, or a mutant thereof, in a biological sample or in a patient. In certain embodiments, a composition of this invention is formulated for administration to a patient in need of such composition. In some embodiments, a composition of this invention is formulated for oral administration to a patient.

The term “patient” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or prodrug of a compound of this invention that, upon administration to a recipient, is capable of providing, either directly or indirectly, a compound of this invention or an inhibitory active metabolite or residue thereof. In some embodiments, a “pharmaceutically acceptable derivative” is a pharmaceutically acceptable salt.

As used herein, the term “inhibitory active metabolite or residue thereof” means that a metabolite or residue thereof is also an inhibitor of STAT6 protein, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### EXEMPLIFICATION

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

TABLE 3
Analytical instruments

| | |
|---|---|
| LCMS | Shimadzu UFLC MS: LCMS-2020 |
| | Agilent Technologies 1200 series MS: Agilent |
| | Technologies 6110 |
| | Agilent Technologies 1200 series MS: LC/MSD VL |
| NMR | BRUKER AVANCE III/400; Frequency (MHz) |
| | 400.13; Nucleus: 1H; Number of Transients: 8 |
| Prep-HPLC | Gilson GX-281 systems: instruments GX-A, GX-B, |
| | GX-C, GX-D, GX-E, GX-F, GX-G and GX-H |
| GCMS | SHIMADZU GCMS-QP2010 Ultra |
| Analytical cSFC | Agilent Technologies 1290 Infinity |
| Prep-cSFC | Waters SFC Prep 80 |

For acidic LCMS data: LCMS was recorded on an Agilent 1200 Series LC/MSD or Shimadzu LCMS2020 equipped with electro-spray ionization and quadruple MS detector [ES+ve to give MH+] and equipped with Chromolith Flash RP-18e 25*2.0 mm, eluting with 0.0375 vol % TFA in water (solvent A) and 0.01875 vol % TFA in acetonitrile (solvent B). Other LCMS was recorded on an Agilent 1290 Infinity RRLC attached with Agilent 6120 Mass detector. The column used was BEH C18 50*2.1 mm, 1.7 micron. Column flow was 0.55 ml/min and mobile phase were used (A) 2 mM Ammonium Acetate in 0.1% Formic Acid in Water and (B) 0.1% Formic Acid in Acetonitrile.

For basic LCMS data: LCMS was recorded on an Agilent 1200 Series LC/MSD or Shimadzu LCMS 2020 equipped with electro-spray ionization and quadruple MS detector [ES+ve to give MH+] and equipped with Xbridge C18, 2.1×50 mm columns packed with 5 mm C18-coated silica or Kinetex EVO C18 2.1×30 mm columns packed with 5 mm C18-coated silica, eluting with 0.05 vol % NH3·H₂O in water (solvent A) and acetonitrile (solvent B).

HPLC Analytical Method: HPLC was carried out on X Bridge C18 150*4.6 mm, 5 micron. Column flow was 1.0 ml/min and mobile phase were used (A) 0.1% Ammonia in water and (B) 0.1% Ammonia in Acetonitrile.

Prep HPLC Analytical Method: The compound was purified on Shimadzu LC-20AP and UV detector. The column used was X-BRIDGE C18 (250*19) mm, 5μ. Column flow was 16.0 ml/min. Mobile phase were used (A) 0.1% Formic Acid in Water and (B) Acetonitrile Basic method used (A) 5 mM ammonium bicarbonate and 0.1% NH3 in Water and (B) Acetonitrile or (A) 0.1% Ammonium Hydroxide in Water and (B) Acetonitrile. The UV spectra were recorded at 202 nm & 254 nm.

NMR Method: The 1H NMR spectra were recorded on a Bruker Ultra Shield Advance 400 MHz/5 mm Probe (BBFO). The chemical shifts are reported in part-per-million.

In some instances, intermediates and compounds described in the examples comprise one or more stereocenters and more than one enantiomer/diastereomer was produced. In some embodiments, these enantiomers/diastereomers were separated and isolated, although stereochemistry was not resolved. Unless otherwise stated, stereochemistry was assigned arbitrarily. For intermediates, each enantiomer/diastereomer with arbitrarily assigned stereochemistry may result in a final compound (e.g., assigned a “I-” number), which also maintains the arbitrarily assigned stereochemistry. Accordingly, any compound with arbitrarily assigned stereochemistry or produced from an intermediate with arbitrarily assigned stereochemistry may be depicted herein as a certain stereoisomer, but it is understood that such compound may be the other stereoisomer (i.e., enantiomer or diastereomer).

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Intermediates:

### 3-(1H-1,2,3-triazol-1-yl)propanoic acid (Intermediate A)

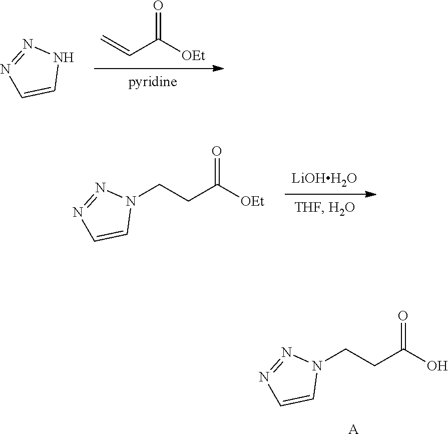

### Step 1—Ethyl 3-(1H-1,2,3-triazol-1-yl)propanoate

To a solution of 1H-1,2,3-triazole (30.0 g, 434 mmol, 25.2 mL, CAS #288-35-7) in ethyl acrylate (43.5 g, 434 mmol, 47.2 mL, CAS #140-88-5) was added pyridine (3.44 g, 43.4 mmol, 3.51 mL). The mixture was then stirred at 90° C. for 4 h. On completion, the mixture was concentrated under vacuum to give the crude product. The crude product was purified by reversed-phase column (C18, 40 g; condition: water/acetonitrile=1/0 to 0/1, 0.1% formic acid) and lyophilized. The title compound (28.9 g, 39% yield) was obtained as a yellow oil. ¹H NMR (400 MHz, CDCl₃) δ=7.68 (s, 1H), 7.66 (s, 1H), 4.69 (t, J=6.4 Hz, 2H), 4.22-4.07 (m, 2H), 2.97 (t, J=6.4 Hz, 2H), 1.30-1.18 (m, 3H).

### Step 2—3-(1H-1,2,3-triazol-1-yl)propanoic acid

To a solution of ethyl 3-(1H-1,2,3-triazol-1-yl)propanoate (15.0 g, 88.7 mmol) in THF (75.0 mL) and H₂O (75.0 mL) was added LiOH·H₂O (7.44 g, 177 mmol). The mixture was then stirred at 25° C. for 12 hr. On completion, the pH of mixture was adjusted to around 5 by adding HCl (1 M). The mixture was then lyophilized to afford the title compound (19.5 g, 99% yield) as a white solid. ¹H NMR (400 MHz, DMSO-d₆) δ=8.08 (s, 1H), 7.65 (s, 1H), 4.49 (t, J=7.2 Hz, 2H), 2.52-2.48 (m, 2H).

### 1-(5-(4,4,5,5-tetramethyl-1,3,2-dioxaborolan-2-yl)-3,6-dihydropyridin-1(2H)-yl)-3-(1H-1,2,3-triazol-1-yl)propan-1-one ((Intermediate B)

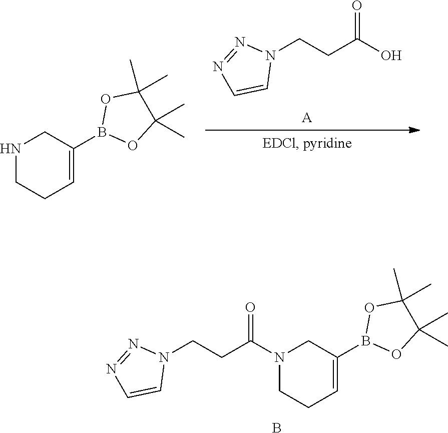

A mixture of 3-(1H-1,2,3-triazol-1-yl)propanoic acid (24.9 g, 114 mmol, Intermediate A), 5-(4,4,5,5-tetramethyl-1,3,2-dioxaborolan-2-yl)-1,2,3,6-tetrahydropyridine (28.1 g, 114 mmol, HCl, synthesized via Step 1 of Intermediate U), and EDCI (24.1 g, 126 mmol) in pyridine (280 mL) was stirred at 25° C. for 1 h. On completion, the mixture was concentrated and purified by prep-HPLC (column: Welch Ultimate XB-CN 250*70*10 um;mobile phase: [Hexane-EtOH];B %: 8%-41%, 30 min) to give the title compound (30.0 g, 71% yield) as a yellow gum. LC-MS (ESI⁺) m/z 333.0 (M+H)⁺; ¹H NMR (400 MHz, CDCl3) δ ppm 7.85-7.55 (m, 2H), 6.69 (d, J=2.0 Hz, 1H), 4.77 (dt, J=2.8, 6.0 Hz, 2H), 4.20-3.91 (m, 2H), 3.54-3.48 (m, 2H), 3.10-2.94 (m, 2H), 2.21 (s, 2H), 1.25 (s, 12H).

### 4-Bromo-6-chloro-7-fluoro-N,N-dimethyl-1H-indole-2-carboxamide (Intermediate C)

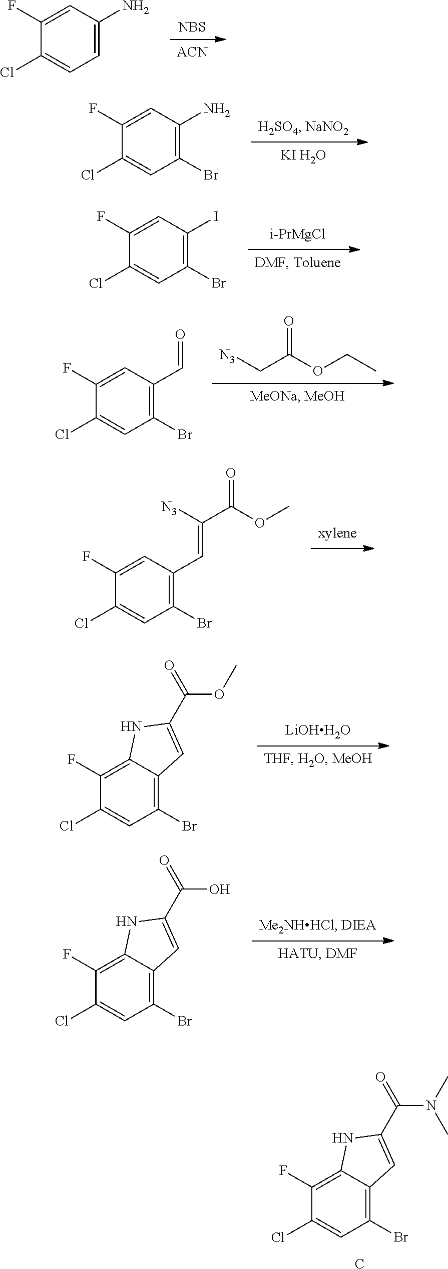

### Step 1—2-Bromo-4-chloro-5-fluoroaniline

To a solution of 4-chloro-3-fluoroaniline (100 g, 687 mmol, CAS #367-22-6) in ACN (1500 mL) was added NBS (122 g, 687 mmol, CAS #128-08-5) at 0° C. The mixture was then stirred at 25° C. for 12 h. On completion, the mixture was added H₂O (800 mL) and it was extracted with ethyl acetate (1000 mL×3), dried over Na₂SO₄ and evaporated. The residue was purified by column chromatography (SiO₂, Petroleum ether/Ethyl acetate=1/1 to 50/1) to give the title compound (83.1 g, 54% yield) as a white solid. ¹H NMR (400 MHz, CDCl₃) δ=7.43 (d, J=7.6 Hz, 1H), 6.57 (d, J=10.4 Hz, 1H), 4.19 (br s, 2H).

### Step 2—1-Bromo-5-chloro-4-fluoro-2-iodobenzene

A solution of 2-bromo-4-chloro-5-fluoroaniline (20.0 g, 89.1 mmol) in H₂O (640 mL) and H₂SO₄ (169 g, 1.69 mol, 92.1 mL) was cooled to 0° C. Next, a solution of NaNO₂ (6.76 g, 98.0 mmol) in H₂O (27 mL) was added and the mixture was stirred for 1 h. Then a solution of KI (19.4 g, 117 mmol) in H₂O (92 mL) was added and the reaction mixture was stirred at 25° C. for 12 h. On completion, ethyl acetate was added and the phases were separated. The aqueous phase was extracted with ethyl acetate (50 mL×3). The combined organic phases are washed with 1 N NaOH (50 mL), 1 N sodium thiosulfate (50 mL), 1 N HCl (30 mL), NaHCO₃ (50 mL) and brine (5 mL), then dried over sodium sulfate, filtered and concentrated under reduced pressure. The residue was purified by column chromatography (SiO₂, Petroleum ether:Ethyl acetate=1:0 to 25:1) to give the title compound (20.2 g, 68% yield) as a white solid. ¹H NMR (400 MHz, CDCl₃) δ=7.65 (dd, J=7.6, 8.8 Hz, 2H), 1.27 (s, 1H), 0.93-0.82 (m, 1H).

### Step 3—2-Bromo-4-chloro-5-fluorobenzaldehyde

A solution of the 1-bromo-5-chloro-4-fluoro-2-iodobenzene (20.2 g, 60.2 mmol) in toluene (180 mL) was cooled to −30° C. Then chloro(isopropyl)magnesium (2 M, 45.2 mL) was added slowly over 0.5 h at −30° C. and the mixture was stirred at this temperature for 1 h. Next, anhydrous DMF (17.6 g, 241 mmol, 18.5 mL) was added slowly over 30 min. Then the reaction mixture was warmed to 0° C. and stirred for 1 h. On completion, the reaction mixture was quenched by addition of NH₄Cl (60 mL) and extracted with EtOAc (60 mL×3). The combined organic layers were washed with brine (50 mL×3), dried over Na₂SO₄ and evaporated to give the title compound (14.5 g) as a white solid. ¹H NMR (400 MHz, CDCl₃) δ=10.25 (d, J=3.2 Hz, 1H), 7.76 (d, J=6.4 Hz, 1H), 7.71 (d, J=8.8 Hz, 1H).

### Step 4—Methyl (Z)-2-azido-3-(2-bromo-4-chloro-5-fluorophenyl)acrylate

To a solution of 2-bromo-4-chloro-5-fluorobenzaldehyde (14.5 g, 61.1 mmol) in MeOH (150 mL) was added dropwise NaOMe (44.0 g, 244 mmol, 30% solution) at −60° C. for 45 min. After addition, the mixture was stirred at this temperature for 1 h, and then ethyl 2-azidoacetate (31.5 g, 244 mmol, 28.0 mL, CAS #637-81-0) was added dropwise at −60° C. for 45 min. The resulting mixture was stirred at −60 to 25° C. over 12 h. On completion, the mixture was cooled to 0° C. and filtered. The filter cake was dried under reduced pressure. The residue was purified by column chromatography (SiO₂, Petroleum ether:Ethyl acetate=1:0-25:1) to give the title compound (8.70 g, 37% yield) as a white solid. ¹H NMR (400 MHz, CDCl₃) δ=8.09 (d, J=10.8 Hz, 1H), 7.66 (d, J=7.2 Hz, 1H), 7.13 (s, 1H), 3.96 (s, 3H).

### Step 5—Methyl 4-bromo-6-chloro-7-fluoro-1H-indole-2-carboxylate

To a solution of methyl (Z)-2-azido-3-(2-bromo-4-chloro-5-fluorophenyl)acrylate (3.17 g, 7.58 mmol) in xylene (30 mL) was reflux at 160° C. for 2 h. The mixture was then cooled to 0° C. and solid formed, which was filtered. The filter cake was dried under reduced pressure to give the title compound (667 mg) as a white solid. ¹H NMR (400 MHz, DMSO-d₆) δ=13.13 (br s, 1H), 7.52 (d, J=5.6 Hz, 1H), 7.09 (d, J=2.8 Hz, 1H), 3.90 (s, 3H).

### Step 6—4-Bromo-6-chloro-7-fluoro-1H-indole-2-carboxylic acid

To a solution methyl 4-bromo-6-chloro-7-fluoro-1H-indole-2-carboxylate (667 mg, 2.18 mmol) in THF (6 mL), H₂O (1.5 mL), and MeOH (1.5 mL) was added LiOH·H₂O (274 mg, 6.53 mmol), then the mixture stirred at 40° C. for 12 h. On completion, the reaction mixture was diluted by addition of H₂O (2 mL), and then extracted with DCM (6 mL×3). The combined aqueous phase was treated with HCl to adjusted pH=3-4, then the mixture was extracted by DCM (4 mL×3). The combined organic phase was concentrated under reduced pressure to give the title compound (620 mg) as a white solid. ¹H NMR (400 MHz, DMSO-d₆) δ=13.12-12.76 (m, 1H), 7.50 (d, J=5.6 Hz, 1H), 7.03 (d, J=2.8 Hz, 1H).

### Step 7—4-Bromo-6-chloro-7-fluoro-N,N-dimethyl-1H-indole-2-carboxamide

To a solution of 4-bromo-6-chloro-7-fluoro-1H-indole-2-carboxylic acid (620 mg, 2.12 mmol) and N-methylmethanamine hydrochloride (259 mg, 3.18 mmol, CAS #506-59-2) in DMF (2 mL) was added DIEA (2.74 g, 21.2 mmol, 3.69 mL) and HATU (967 mg, 2.54 mmol). The mixture was then stirred at 0° C. for 2 h. On completion, the mixture was poured into H₂O (10 mL) and a solid precipitated. The solid was filtered and concentrated to give the title compound (617 mg, 91% yield) as a white solid. ¹H NMR (400 MHz, DMSO-d₆) δ=12.70 (br s, 1H), 7.46 (d, J=5.6 Hz, 1H), 6.79 (d, J=2.8 Hz, 1H), 3.29-3.05 (m, 10H).

### 6-(1-(3-(1H-1,2,3-triazol-1-yl)propanoyl)-1,2,5,6-tetrahydropyridin-3-yl)-7-fluoro-4-(2-methoxyphenyl)-1H-indole-2-carboxylic acid (Intermediate D)

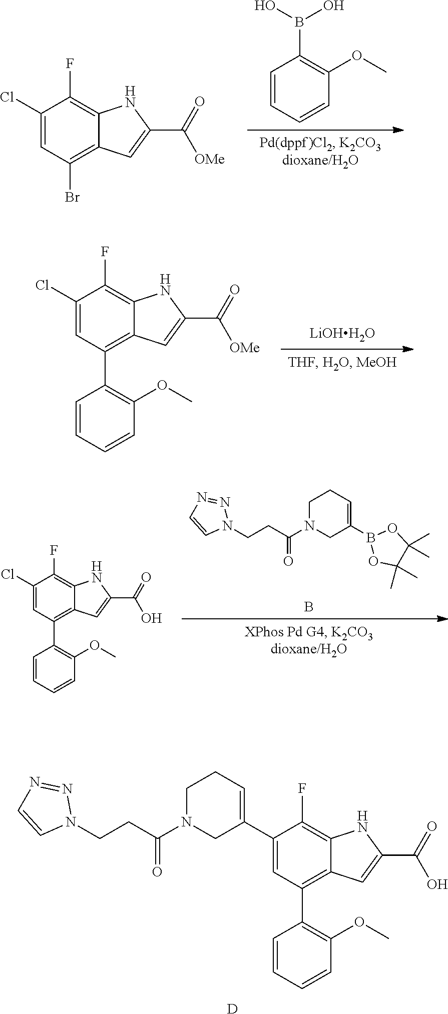

### Step 1—Methyl 6-chloro-7-fluoro-4-(2-methoxyphenyl)-1H-indole-2-carboxylate

To a solution of methyl 4-bromo-6-chloro-7-fluoro-1H-indole-2-carboxylate (13.6 g, 44.4 mmol, synthesized via Steps 1-5 of Intermediate C), (2-methoxyphenyl)boronic acid (13.5 g, 88.7 mmol, CAS #1072951-73-5) and K₂CO₃ (18.4 g, 133 mmol) in dioxane (150 mL) and H₂O (30 mL) was added Pd(dppf)Cl₂ (3.25 g, 4.44 mmol) under N₂. The mixture was then stirred at 80° C. for 2 h. On completion, the reaction mixture was diluted with water (200 mL) and extracted with EtOAc (200 mL×3). The combined organic layers were washed with brine (80 mL×3), dried over Na₂SO₄ and evaporated. The crude product was triturated with PE/EA=10/1 (100 mL) at 25° C. for 30 min to afford the title compound (14.0 g, 95% yield) as a black solid. ¹H NMR (400 MHz, DMSO-d₆) δ=12.74 (s, 1H), 7.46-7.39 (m, 1H), 7.31 (dd, J=1.6, 7.2 Hz, 1H), 7.17 (d, J=8.4 Hz, 1H), 7.11 (d, J=6.0 Hz, 1H), 7.06 (t, J=7.6 Hz, 1H), 6.83 (d, J=2.8 Hz, 1H), 3.85 (s, 3H), 3.72 (s, 3H).

### Step 2—6-Chloro-7-fluoro-4-(2-methoxyphenyl)-1H-indole-2-carboxylic acid

To a solution of methyl 6-chloro-7-fluoro-4-(2-methoxyphenyl)-1H-indole-2-carboxylate (14.0 g, 42.0 mmol) in THF (50 mL), H₂O (50 mL), and MeOH (50 mL) was added LiOH·H₂O (8.80 g, 210 mmol). The mixture was then stirred at 40° C. for 2 h. On completion, the reaction mixture was evaporated and diluted with H₂O (100 mL). The pH was adjusted to 4-3 by addition of 2 M HCl. The mixture was filtered and the filter cake was washed with water (50 mL×2). Then the filter cake was dried to afford the title compound (14 g) as a brown solid. LC-MS (ESI⁺) m/z 320.0 (M+H)⁺.

### Step 3—6-(1-(3-(1H-1,2,3-triazol-1-yl)propanoyl)-1,2,5,6-tetrahydropyridin-3-yl)-7-fluoro-4-(2-methoxyphenyl)-1H-indole-2-carboxylic acid

To a solution of 6-chloro-7-fluoro-4-(2-methoxyphenyl)-1H-indole-2-carboxylic acid (6.00 g, 18.8 mmol), and 1-(5-(4,4,5,5-tetramethyl-1,3,2-dioxaborolan-2-yl)-3,6-dihydropyridin-1(2H)-yl)-3-(1H-1,2,3-triazol-1-yl)propan-1-one (7.48 g, 22.5 mmol, Intermediate B) in dioxane (60 mL) and H₂O (12 mL) was added Xphos Pd G4 (1.61 g, 1.88 mmol), and K₂CO₃ (7.78 g, 56.3 mmol) under N₂. The mixture was then stirred at 100° C. for 2 h. On completion, the reaction mixture was evaporated, diluted with water (300 mL) and extracted with DCM (300 mL×2). The pH of aqueous phase was adjusted to 4-3 by addition of 2 M HCl. Then the mixture was filtered and the filter cake was washed with water (300 mL×2) and dried. Then the crude product was triturated with PE/EA/DCM=10/1/1 (240 mL) at 25° C. for 30 min to afford the title compound (16 g, 87% yield) as a grey solid. LC-MS (ESI⁺) m/z 490.2 (M+H)⁺; ¹H NMR (400 MHz, DMSO-d₆) δ=13.07 (s, 1H), 12.30 (dd, J=1.6, 8.4 Hz, 1H), 8.14-8.08 (m, 1H), 7.71-7.65 (m, 1H), 7.41 (t, J=7.6 Hz, 1H), 7.32 (dd, J=1.6, 7.6 Hz, 1H), 7.15 (d, J=8.4 Hz, 1H), 7.05 (t, J=7.2 Hz, 1H), 6.99-6.90 (m, 1H), 6.75 (br s, 1H), 6.18-6.10 (m, 1H), 5.76 (s, 1H), 4.66-4.56 (m, 2H), 4.39-4.28 (m, 2H), 3.74-3.69 (m, 3H), 3.67-3.57 (m, 2H), 3.13-3.05 (m, 2H), 2.37-2.25 (m, 2H).

### 1-(3-(Trifluoromethoxy)pyridin-2-yl)piperazine (Intermediate E)

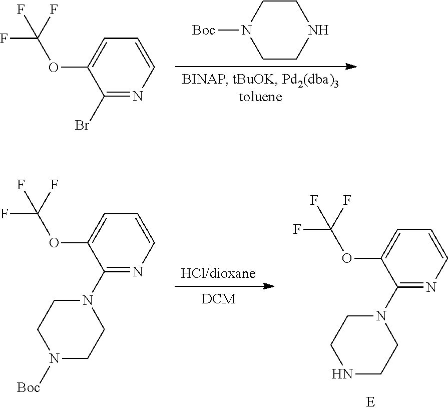

### Step 1—Tert-butyl 4-(3-(trifluoromethoxy)pyridin-2-yl)piperazine-1-carboxylate

To a solution of 2-bromo-3-(trifluoromethoxy)pyridine (500 mg, 2.07 mmol, CAS #1206978-11-1) and tert-butyl piperazine-1-carboxylate (577 mg, 3.10 mmol, CAS #57260-71-6) in toluene (5 mL) was added BINAP (193 mg, 310 μmol), tBuOK (348 mg, 3.10 mmol) and Pd₂(dba)₃ (189 mg, 207 mol) at 25° C. Then the mixture was stirred at 110° C. for 12 hrs. On completion, the reaction mixture was concentrated in vacuo to get the crude residue. The residue was purified by column chromatography (SiO₂, Petroleum ether/Ethyl acetate=0/1 to 10/1) to give the title compound (580 mg, 45% yield) as a yellow oil. LC-MS (ESI⁺) m/z 291.9 (M−55)⁺.

### Step 2: 1-(3-(trifluoromethoxy)pyridin-2-yl)piperazine

To a solution of tert-butyl 4-(3-(trifluoromethoxy)pyridin-2-yl)piperazine-1-carboxylate (400 mg, 1.15 mmol) in DCM (1 mL) was added HCl/dioxane (4 M, 287.91 μL) at 25° C., then the mixture was stirred at 25° C. for 1 hr. On completion, the reaction mixture was concentrated in vacuo to give the title compound (350 mg, HCl) as a yellow solid. LC-MS (ESI⁺) m/z 247.9 (M+H)⁺.

### 1-(3-Isopropoxypyridin-2-yl)piperazine (Intermediate F)

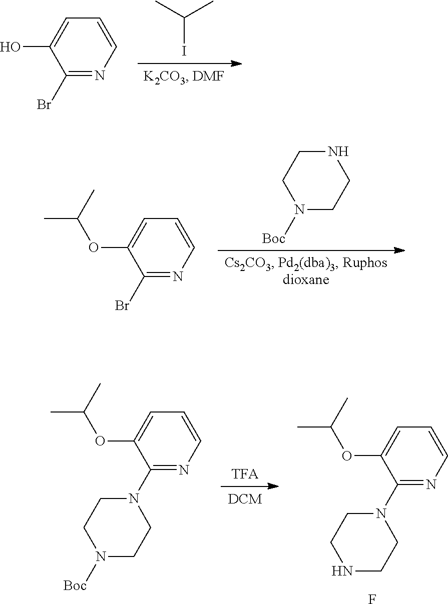

### Step 1—2-Bromo-3-isopropoxypyridine

To a solution of 2-iodopropane (976 mg, 5.75 mmol, CAS #75-30-9) and 2-bromopyridin-3-ol (500 mg, 2.87 mmol, CAS #6602-32-0) in anhydrous DMF (10 mL) was added K₂CO₃ (1.19 g, 8.62 mmol) at 25° C. Then the reaction was stirred at 80° C. for 2 hrs. On completion, the reaction mixture was quenched with water (10 mL) and extracted with ethyl acetate (3×10 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether/Ethyl acetate=5/1) to give the title compound (600 mg, 97% yield) as a yellow oil. LC-MS (ESI⁺) m/z 216.0 (M+H)⁺.

### Step 2—Tert-butyl 4-(3-isopropoxypyridin-2-yl)piperazine-1-carboxylate

To a solution of 2-bromo-3-isopropoxypyridine (300 mg, 1.39 mmol) and tert-butyl piperazine-1-carboxylate (258 mg, 1.39 mmol) in dioxane (3 mL) was added Cs₂CO₃ (1.36 g, 4.17 mmol), RuPhos (129 mg, 277 μmol) and Pd₂(dba)₃ (127 mg, 138 μmol) at 25° C. under nitrogen atmosphere. Then the mixture was stirred at 100° C. for 12 hrs under nitrogen atmosphere. On completion, the reaction mixture was concentrated in vacuo to give the crude residue. The residue was purified by column chromatography (SiO₂, Petroleum ether/Ethyl acetate=8/1) to give the title compound (500 mg, 93% yield) as a brown solid. LC-MS (ESI⁺) m/z 321.9 (M+H)⁺.

### Step 3—1-(3-Isopropoxypyridin-2-yl)piperazine

To a solution of tert-butyl 4-(3-isopropoxypyridin-2-yl)piperazine-1-carboxylate (250 mg, 777 μmol) in anhydrous DCM (5 mL) was added TFA (3.07 g, 26.9 mmol, 2 mL) at 25° C., then the reaction was stirred at 25° C. for 1 hr. On completion, the reaction mixture was filtered and the filtrate was concentrated in vacuo to give the title compound (200 mg, TFA) as a yellow oil. LC-MS (ESI⁺) m/z 196.1 (M+H)⁺.

### 1-(3-Cyclopropoxypyridin-2-yl)piperazine (Intermediate G)

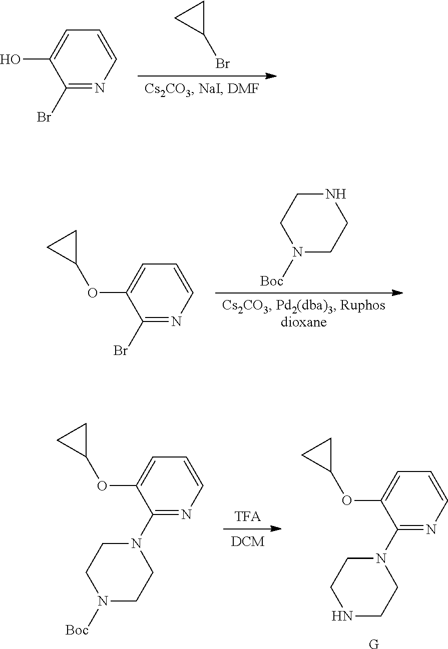

To a solution of 2-bromopyridin-3-ol (1 g, 6 mmol, CAS #6602-32-0) and bromocyclopropane (1.39 g, 11.4 mmol, CAS #4333-56-6) in anhydrous DMF (10 mL) was added Cs₂CO₃ (3.75 g, 11.4 mmol) and KI (95.4 mg, 574 μmol) at 25° C. Then the reaction was stirred at 140° C. for 1 hr under microwave. On completion, the reaction mixture was quenched with water (15 mL) and extracted with ethyl acetate (3×10 mL). The combined organic layers were washed with brine (15 mL) and dried over anhydrous sodium sulfate, filtered and the filtrate was concentrated in vacuo to give the crude residue. The residue was purified by column chromatography (SiO₂, Petroleum ether/Ethyl acetate=8/1) to give the title compound (80 mg, 6% yield) as a yellow solid. LC-MS (ESI⁺) m/z 213.8 (M+H)⁺.

### Step 2—Tert-butyl 4-(3-cyclopropoxypyridin-2-yl)piperazine-1-carboxylate

To a solution of 2-bromo-3-cyclopropoxypyridine (70 mg, 330 μmol) and tert-butyl piperazine-1-carboxylate (67.0 mg, 359 μmol, CAS #57260-71-6) in dioxane (1 mL) was added Cs₂CO₃ (319 mg, 981 μmol), Pd₂(dba)₃ (29.9 mg, 32.7 μmol) and RuPhos (30.5 mg, 65.4 μmol) at 25° C. under nitrogen atmosphere. Then the mixture was stirred at 100° C. for 12 hrs under nitrogen atmosphere. On completion, the reaction mixture was concentrated in vacuo to give the crude residue. The residue was purified by column chromatography (SiO₂, Petroleum ether/Ethyl acetate=7/1) to give the title compound (70 mg, 54% yield) as a yellow oil. LC-MS (ESI⁺) m/z 320.1 (M+H)⁺.

### Step 3—1-(3-Cyclopropoxypyridin-2-yl)piperazine

To a solution of tert-butyl 4-(3-cyclopropoxypyridin-2-yl)piperazine-1-carboxylate (60 mg, 190 μmol) in anhydrous DCM (2 mL) was added TFA (263 mg, 2.31 mmol) at 25° C., then the reaction was stirred at 25° C. for 1 hr. On completion, the reaction mixture was concentrated in vacuo to give the title compound (50 mg, TFA) as a yellow oil. LC-MS (ESI⁺) m/z 220.0 (M+H)⁺.

### 1-(3-Isopropypyridin-2-yl)piperazine (Intermediate H)

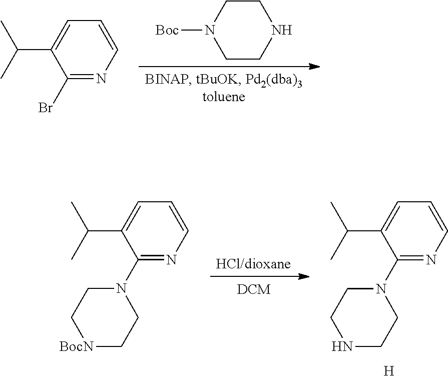

### Step 1—Tert-butyl 4-(3-isopropylpyridin-2-yl)piperazine-1-carboxylate

To a solution of 2-bromo-3-isopropylpyridine (800 mg, 4.00 mmol, CAS #38282560) and tert-butyl piperazine-1-carboxylate (1.12 g, 6.00 mmol, CAS #57260-71-6) in dioxane (2 mL) was added Cs₂CO₃ (3.91 g, 12.0 mmol) and RuPhos (373 mg, 800 μmol) and Pd₂(dba)₃ (366 mg, 400 μmol) at 25° C., then the mixture was stirred at 100° C. for 2 hrs. On completion, the reaction mixture was concentrated in vacuo to get the crude residue. The residue was purified by column chromatography (SiO₂, Petroleum ether/Ethyl acetate=30/1 to 10/1) to give the title compound (580 mg, 32% yield) as a yellow oil. LC-MS (ESI⁺) m/z 376.1 (M+H)⁺.

### Step 2—1-(3-Isopropylpyridin-2-yl)piperazine

To a solution of tert-butyl 4-(3-isopropylpyridin-2-yl)piperazine-1-carboxylate (250 mg, 819 μmol) in DCM (3 mL) was added HCl/dioxane (4 M, 1 mL) at 25° C., then the mixture was stirred at 25° C. for 2 hrs. On completion, the reaction mixture was concentrated in vacuo to give the title compound (300 mg, HCl) as a yellow solid. LC-MS (ESI⁺) m/z 376.1 (M+H)⁺.

### 1-(3-Phenoxypyridin-2-yl)piperazine (Intermediate I)

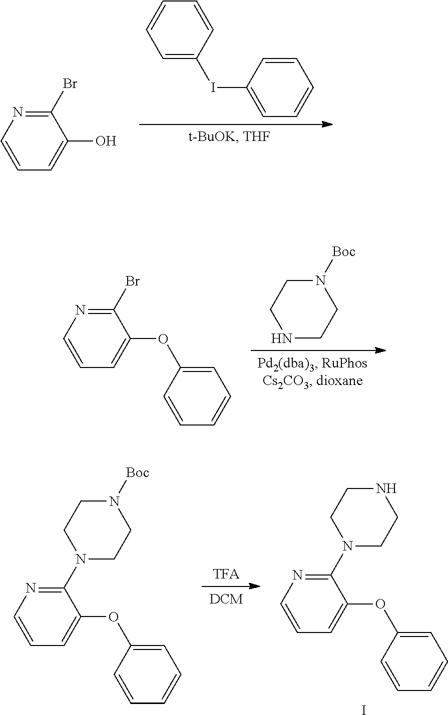

### Step 1—2-Bromo-3-phenoxypyridine

A mixture of 2-bromopyridin-3-ol (480 mg, 2.76 mmol), diphenyliodonium iodide (1.13 g, 2.76 mmol, CAS #6602-32-0), diphenyl-12-iodane (331 mg, 2.95 mmol, CAS #2217-79-0) in THF (5 mL) was degassed and purged with N₂ three times. Then the mixture was stirred at 80° C. for 3 hrs under N₂ atmosphere. On completion, the reaction mixture was quenched with water (30 mL) and extracted with EA (3×30 mL). The combined organic layers were washed with aqueous NaCl (30 mL) and dried over anhydrous sodium sulfate, filtered and the filtrate was concentrated in vacuo to give the crude residue. The residue was purified by column chromatography (SiO₂, Petroleum ether:Ethyl acetate=1/0) to give the title compound (450 mg, 65% yield) as a white solid. LC-MS (ESI⁺) m/z 251.8 (M+H)⁺.

### Step 2—Tert-butyl 4-(3-phenoxypyridin-2-yl)piperazine-1-carboxylate

A mixture of 2-bromo-3-phenoxypyridine (440 mg, 1.76 mmol), tert-butyl piperazine-1-carboxylate (492 mg, 2.64 mmol, CAS #57260-71-6), Pd₂(dba)₃ (161 mg, 176 μmol), RuPhos (82.1 mg, 176 μmol) and Cs₂CO₃ (1.72 g, 5.28 mmol) in dioxane (5 mL) was degassed and purged with N₂ three times. Then the mixture was stirred at 100° C. for 12 hrs under N₂ atmosphere. On completion, the reaction mixture was quenched with water (30 mL) and extracted with EA (3×30 mL). The combined organic layers were washed with aqueous NaCl (30 mL) and dried over anhydrous sodium sulfate, filtered and the filtrate was concentrated in vacuo to give the crude residue. The residue was purified by column chromatography (SiO₂, Petroleum ether:Ethyl acetate=19/1) to give the title compound (340 mg, 54% yield) as a yellow solid. LC-MS (ESI⁺) m/z 355.8 (M+H)⁺.

### Step 3—1-(3-Phenoxypyridin-2-yl)piperazine

To a solution of tert-butyl 4-(3-phenoxypyridin-2-yl)piperazine-1-carboxylate (340 mg, 957 μmol) in DCM (2 mL) was added TFA (1.54 g, 13.5 mmol) and the mixture was stirred at 25° C. for 1 hr. On completion, the reaction mixture was concentrated under reduced pressure to give the title compound (400 mg, TFA) as a white solid.

### 1-(3-Ethoxypyridin-2-yl)piperazine (Intermediate J)

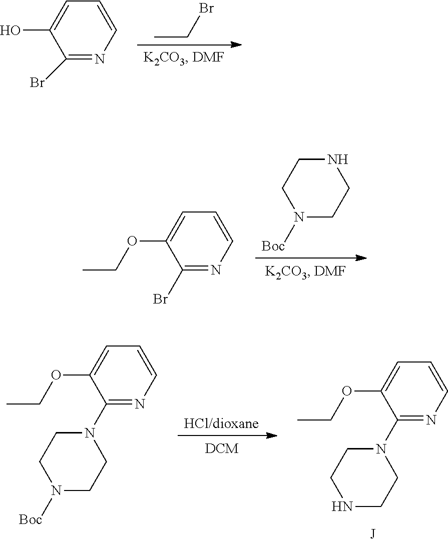

### Step 1—2-Bromo-3-ethoxypyridine

To a solution of 2-bromopyridin-3-ol (500 mg, 2.87 mmol, CAS #6602-32-0), bromoethane (532 mg, 4.89 mmol, CAS #74-96-4) in DMF (3 mL) was added K₂CO₃ (794 mg, 5.75 mmol). The mixture was then stirred at 80° C. for 2 hrs. On completion, the reaction mixture was quenched with water (30 mL) and extracted with EA (3×30 mL). The combined organic layers were washed with aqueous NaCl (30 mL) and dried over anhydrous sodium sulfate, filtered and the filtrate was concentrated in vacuo to give the title compound (400 mg) as a yellow oil solid. LC-MS (ESI⁺) m/z 203.9 (M+H)⁺.

### Step 2—2-(2-Nitrophenyl)acetaldehyde

A mixture of 2-bromo-3-ethoxypyridine (350 mg, 1.73 mmol), tert-butyl piperazine-1-carboxylate (484 mg, 2.60 mmol, CAS #57260-71-6), RuPhos Pd G3 (145 mg, 173 μmol), and Cs₂CO₃ (1.13 g, 3.46 mmol) in dioxane (5 mL) was degassed and purged with N₂ three times. Then the mixture was stirred at 100° C. for 2 hrs under N₂ atmosphere. On completion, the reaction mixture was quenched with water (30 mL) and extracted with EA (3×30 mL). The combined organic layers were washed with aqueous NaCl (30 mL) and dried over anhydrous sodium sulfate, filtered and the filtrate was concentrated in vacuo to give the crude residue. The crude residue was purified by reversed-phase HPLC (0.1% FA condition) to give the title compound (450 mg, 85% yield) as a brown solid. LC-MS (ESI⁺) m/z 252.0 (M+H-56)⁺.

### Step 3—1-(3-ethoxypyridin-2-yl)piperazine

To a solution of tert-butyl 4-(3-ethoxypyridin-2-yl)piperazine-1-carboxylate (450 mg, 1.46 mmol) in DCM (10 mL) was added TFA (3.07 g, 26.9 mmol, 2 mL). The mixture was then stirred at 25° C. for 12 hrs. On completion, the reaction mixture was quenched with water (30 mL) and extracted with EA (3×30 mL). The combined organic layers were washed with aqueous NaCl (30 mL) and dried over anhydrous sodium sulfate, filtered and the filtrate was concentrated in vacuo to give the title compound (400 mg, TFA) as a brown oil solid. LC-MS (ESI⁺) m/z 208.3 (M+H)⁺.

### 6-(1-Acetyl-1,2,5,6-tetrahydropyridin-3-yl)-7-fluoro-N,N-dimethyl-4-(4,4,5,5-tetramethyl-1,3,2-dioxaborolan-2-yl)benzo[b]thiophene-2-carboxamide (Intermediate K)

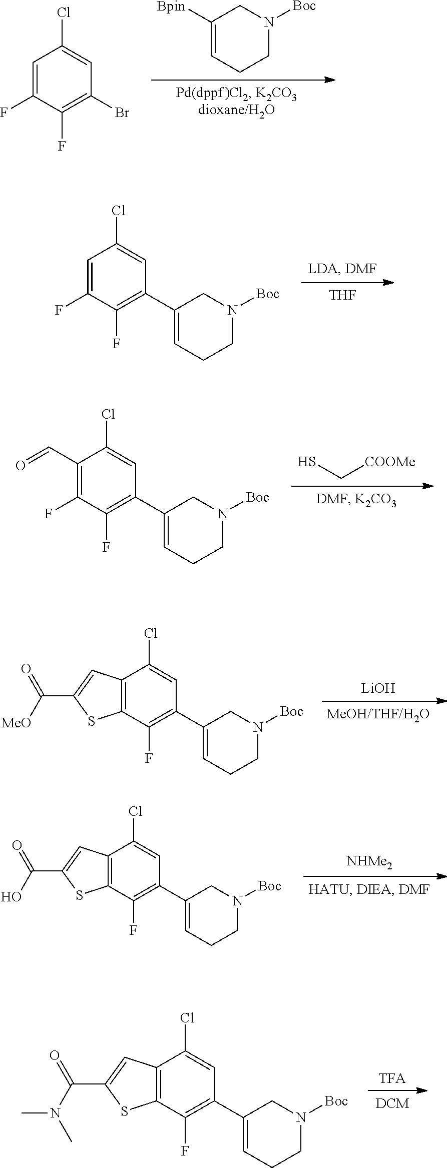

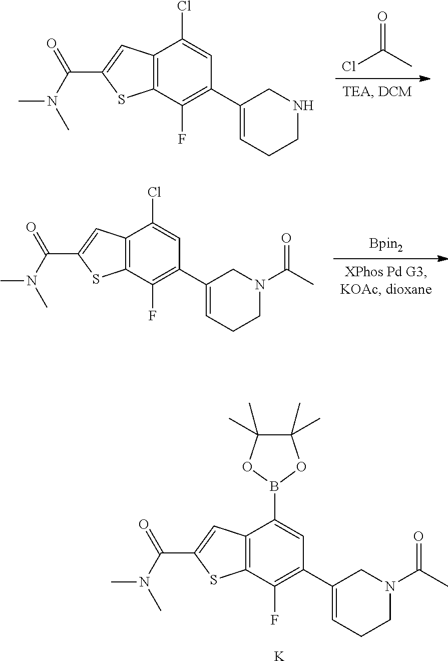

### Step 1—Tert-butyl 5-(5-chloro-2,3-difluorophenyl)-3,6-dihydropyridine-1(2H)-carboxylate

A mixture of 1-bromo-5-chloro-2,3-difluoro-benzene (5 g, 22.0 mmol, CAS #1160573-26-1), tert-butyl 5-(4,4,5,5-tetramethyl-1,3,2-dioxaborolan-2-yl)-3,6-dihydro-2H-pyridine-1-carboxylate (7.48 g, 24.2 mmol CAS #885693-20-9), Pd(dppf)Cl₂·CH₂Cl₂ (1.80 g, 2.20 mmol), and K₂CO₃ (9.12 g, 65.9 mmol) in dioxane (40 mL) and H₂O (10 mL) was degassed and purged with N₂ three times. Then the mixture was stirred at 80° C. for 2 hrs under N₂ atmosphere. On completion, the reaction mixture was diluted with water (10 mL) and extracted with dichloromethane (2×20 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=10/1) to give the title compound (7 g, 96% yield) as a yellow solid. LC-MS (ESI⁺) m/z 274.0 (M−55)⁺.

### Step 2—Tert-butyl 5-(5-chloro-2,3-difluoro-4-formylphenyl)-3,6-dihydropyridine-1(2H)-carboxylate

A mixture of tert-butyl 5-(5-chloro-2,3-difluoro-phenyl)-3,6-dihydro-2H-pyridine-1-carboxylate (6.00 g, 18.2 mmol) in dry THF (50 mL) was degassed and purged with N₂ three times, then LDA (2 M, 10.9 mL) was added at −78° C., then the mixture was stirred at −78° C. for 2 hrs under N₂ atmosphere. Next, DMF (2.66 g, 36.4 mmol) was added and the mixture was stirred at −78° C. for 2 hrs under N₂ atmosphere. On completion, the reaction mixture was quenched with sat. NH₄Cl (10 mL) and extracted with EtOAc (3×50 mL). The combined organic layers were washed with brine (50 mL) and dried over anhydrous sodium sulfate, filtered and the filtrate was concentrated in vacuo to give the residue. The residue was purified by column chromatography (SiO₂, Petroleum ether:Ethyl acetate=10/1) to give the title compound (7 g, 96% yield) as a yellow solid. LC-MS (ESI⁺) m/z 302.0 (M−55)⁺.

### Step 3—Tert-butyl 5-(4-chloro-7-fluoro-2-(methoxycarbonyl)benzo[b]thiophen-6-yl)-3,6-dihydropyridine-1(4)-carboxylate

To a solution of tert-butyl 5-(5-chloro-2,3-difluoro-4-formyl-phenyl)-3,6-dihydro-2H-pyridine-1-carboxylate (4.5 g, 13 mmol) in DMF (80 mL) was added methyl 2-sulfanylacetate (1.60 g, 15.1 mmol, CAS #2365-48-2). Then K₂CO₃ (3.48 g, 25.2 mmol) was added and the mixture was stirred at 20° C. for 4 hrs. On completion, water (160 mL) was added and the mixture was filtered to give the filter cake as the title compound (6 g) as a yellow solid. LC-MS (ESI⁺) m/z 369.8 (M−55)⁺.

### Step 4—6-(1-(Tert-butoxycarbonyl)-1,2,5,6-tetrahydropyridin-3-yl)-4-chloro-7-fluorobenzo[b]thiophene-2-carboxylic acid

To a solution of tert-butyl 5-(4-chloro-7-fluoro-2-methoxycarbonyl-benzothiophen-6-yl)-3,6-dihydro-2H-pyridine-1-carboxylate (5.50 g, 12.9 mmol) in THF (8 mL), MeOH (2 mL) and H₂O (2 mL) was added LiOH·H₂O (1.63 g, 38.7 mmol). Then the mixture was stirred at 40° C. for 2 hrs. On completion, the reaction mixture was added 6M HCl to adjust the pH to 3-4, and then extracted with DCM (10 mL×3). The combined organic layers were washed with brine (10 mL), dried over Na₂SO₄, filtered and concentrated under reduced pressure to give the title compound (5.5 g) as a yellow solid. LC-MS (ESI⁺) m/z 355.8 (M−55)⁺.

### Step 5—Tert-butyl 5-(4-chloro-2-(dimethylcarbamoyl)-7-fluorobenzo[b]thiophen-6-yl)-3,6-dihydropyridine-1(2H)-carboxylate

To a solution of 6-(1-tert-butoxycarbonyl-3,6-dihydro-2H-pyridin-5-yl)-4-chloro-7-fluoro-benzothiophene-2-carboxylic acid (5.00 g, 12.1 mmol) in DMF (30 mL) was added DIEA (7.84 g, 60.7 mmol) and HATU (5.08 g, 13.4 mmol). Then N-methylmethanamine (2.97 g, 36.4 mmol, HCl) was added and the mixture was stirred at 25° C. for 1 hr. On completion, the reaction mixture was quenched by addition of H₂O (10 mL), and then extracted with EtOAc (30 mL×3). The combined organic layers were washed with brine (30 mL×3), dried over Na₂SO₄, filtered and concentrated under reduced pressure to give the title compound (5.5 g) as a yellow solid. LC-MS (ESI⁺) m/z 439.0 (M+H)⁺.

### Step 6—4-Chloro-7-fluoro-N,N-dimethyl-6-(1,2,5,6-tetrahydropyridin-3-yl)benzo[b]thiophene-2-carboxamide

To a solution of tert-butyl 5-[4-chloro-2-(dimethylcarbamoyl)-7-fluoro-benzothiophen-6-yl]-3,6-dihydro-2H-pyridine-1-carboxylate (5.50 g, 12.5 mmol) in DCM (40 mL) was added TFA (15.4 g, 135 mmol). The mixture was then stirred at 25° C. for 1 hr. On completion, the reaction mixture was concentrated under N₂ to remove solvent. The reaction mixture was quenched by addition of sat. NaHCO₃ to adjust the pH to 7-8, and then extracted with DCM (20 mL×3). The combined organic layers were washed with brine (20 mL), dried over Na₂SO₄, filtered and concentrated under reduced pressure to give the title compound (5.0 g) as a yellow solid. LC-MS (ESI⁺) m/z 339.0 (M+H)⁺.

### Step 7—6-(1-Acetyl-1,2,5,6-tetrahydropyridin-3-yl)-4-chloro-7-fluoro-N,N-dimethylbenzo[b]thiophene-2-carboxamide

To a solution of 4-chloro-7-fluoro-N,N-dimethyl-6-(1,2,3,6-tetrahydropyridin-5-yl)benzothiophene-2-carboxamide (3.80 g, 11.2 mmol) in DCM (35 mL) was added TEA (4.54 g, 44.9 mmol). Then acetyl chloride (2.64 g, 33.7 mmol) was added and the mixture was stirred at 0-25° C. for 1 hrs. On completion, the reaction mixture was quenched by addition of H₂O (5 mL), and then extracted with DCM (20 mL×3). The combined organic layers were washed with brine (20 mL), dried over Na₂SO₄, filtered and concentrated under reduced pressure to give a residue. The residue was purified by silica gel column chromatography (Petroleum ether/Ethyl acetate=3/1 to DCM/Ethyl acetate=3/1) to give the title compound (2.3 g, 52% yield) as a yellow solid. LC-MS (ESI⁺) m/z 380.9 (M+H)⁺.

### Step 8—6-(1-Acetyl-1,2,5,6-tetrahydropyridin-3-yl)-7-fluoro-N,N-dimethyl-4-(4,4,5,5-tetramethyl-1,3,2-dioxaborolan-2-yl)benzo[b]thiophene-2-carboxamide

A mixture of 6-(1-acetyl-3,6-dihydro-2H-pyridin-5-yl)-4-chloro-7-fluoro-N,N-dimethyl-benzothiophene-2-carboxamide (2.20 g, 5.78 mmol), 4,4,5,5-tetramethyl-2-(4,4,5,5-tetramethyl-1,3,2-dioxaborolan-2-yl)-1,3,2-dioxaborolane (2.93 g, 11.6 mmol), KOAc (1.70 g, 17.3 mmol), and XPhos Pd G3 (978 mg, 1.16 mmol) in dioxane (15 mL) was degassed and purged with N₂ three times. Then the mixture was stirred at 80° C. for 2 hrs under N₂ atmosphere. On completion, the reaction mixture was filtered and concentrated under reduced pressure to give a residue. The residue was purified by silica gel column chromatography (Petroleum ether/Ethyl acetate=10:1 to DCM/Ethyl acetate=5/1) to give the title compound (1.70 g, 54% yield) as a yellow solid. LC-MS (ESI⁺) m/z 473.3 (M+H)⁺.

### 6-(1-(3-(1H-1,2,3-triazol-1-yl)propanoyl)-1,2,5,6-tetrahydropyridin-3-yl)-4-chloro-7-fluoro-N,N-dimethylbenzo[b]thiophene-2-carboxamide (Intermediate L)

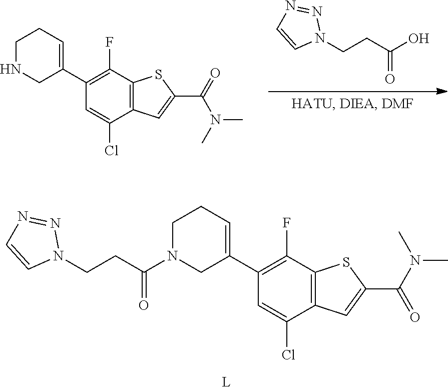 [SMILES: CN(C)C(=O)c1cc2c(Cl)cc(C3=CCCNC3)c(F)c2s1 ; O=C(O)CCn1ccnn1 ; "HATU, DIEA, DMF" ; CN(C)C(=O)c1cc2c(Cl)cc(C3=CCCN(C(=O)CCn4ccnn4)C3)c(F)c2s1]

To a solution of 3-(1H-1,2,3-triazol-1-yl)propanoic acid (250 mg, 1.77 mmol, CAS #4332-45-0) in DMF (10 mL) was added HATU (876 mg, 2.30 mmol), DIEA (1.14 g, 8.86 mmol, 1.54 mL) and 4-chloro-7-fluoro-N,N-dimethyl-6-(1,2,5,6-tetrahydropyridin-3-yl)benzo[b]thiophene-2-carboxamide (802 mg, 1.77 mmol, synthesized via Steps 1-6 of Intermediate K). The mixture was then stirred at 25° C. for 0.5 hrs. On completion, the mixture was concentrated in vacuo to get the residue. The crude residue was purified by reversed-phase HPLC (0.1% FA condition) to give the title compound (400 mg, 44% yield) as a white solid. LC-MS (ESI⁺) m/z 462.1 (M+H)⁺.

### (2-Methoxyphenyl)boronic acid (CAS #5720-06-9)(Intermediate M)

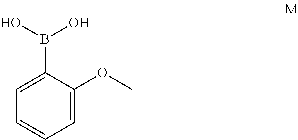

### (6-Chloro-7-fluoro-1H-indol-2-yl)(4-(3-methoxypyridin-2-yl)piperazin-1-yl)methanone (Intermediate N)

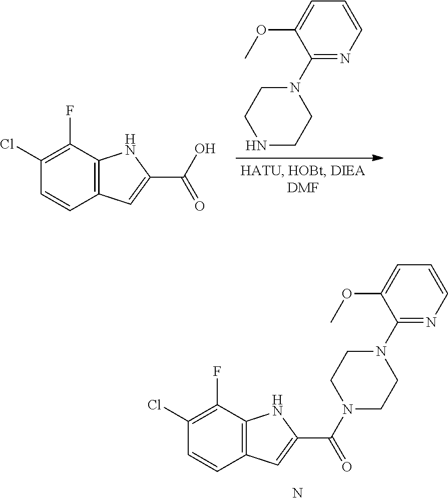

To a solution of 6-chloro-7-fluoro-1H-indole-2-carboxylic acid (200 mg, 936 μmol, CAS #259860-07-6) in DMF (10 mL) was added HATU (392 mg, 1.03 mmol), HOBt (139 mg, 1.03 mmol), DIEA (1.21 g, 9.36 mmol) and 1-(3-methoxy-2-pyridyl)piperazine (575 mg, 1.87 mmol, TFA, CAS #80827-67-4). The mixture was then stirred at 25° C. for 1 hr. On completion, the reaction mixture was diluted with water (30 mL) and extracted with ethyl acetate (3×30 mL). The combined organic layers were washed with brine (60 mL) and dried over anhydrous sodium sulfate, filtered and the filtrate was concentrated in vacuo to give the crude residue. The crude product was purified by column chromatography (SiO2, Petroleum ether:Ethyl acetate=2/1) to give the title compound (400 mg, 96% yield) as a yellow solid. LC-MS (NEG) m/z 389.1 (M+H)⁺.

### 1-(3-Chloropyridin-2-yl)piperazine (CAS #87394-55-6) (Intermediate O)

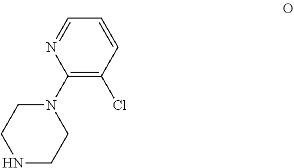 [SMILES: Clc1cccnc1N1CCNCC1]

### 1-Cyclopropylpiperazine (CAS #139256-79-4) (Intermediate P)

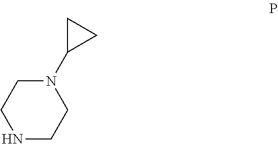

### 1-(3-Methoxypyridin-2-yl)piperazine (CAS #80827-67-4) (Intermediate Q)

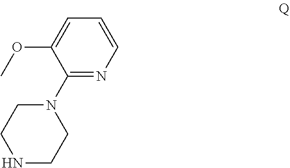

### 1-(3-Ethylpyridin-2-yl)piperazine (Intermediate R)

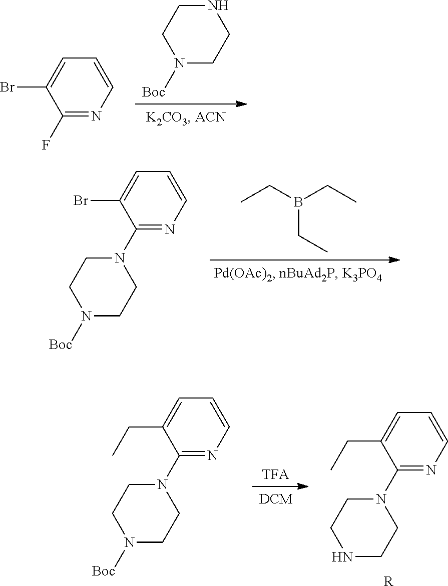

### Step 1—Tert-butyl 4-(3-bromopyridin-2-yl)piperazine-1-carboxylate

To a solution of 3-bromo-2-fluoropyridine (2 g, 11 mmol, CAS #36178-05-9) in DMSO (20 mL) tert-butyl piperazine-1-carboxylate (2.12 g, 11.3 mmol, CAS #57260-71-6) was added K₂CO₃ (3.14 g, 22.7 mmol). The mixture was then stirred at 60° C. for 16 hrs. On completion, the mixture was diluted with water (100 mL) and extracted with dichloromethane (2×100 mL). The combined organic layers were washed with brine (200 mL) and dried over anhydrous sodium sulfate, filtered and the filtrate was concentrated in vacuo to give the crude residue. The crude residue was purified by prep-HPLC (0.1% FA condition) to give the title compound (1.1 g, 28% yield) as a colorless oil. LC-MS (ESI⁺) m/z 285.9 (M−55)⁺.

### Step 2—Tert-butyl 4-(3-ethylpyridin-2-yl)piperazine-1-carboxylate

A mixture of tert-butyl 4-(3-bromopyridin-2-yl)piperazine-1-carboxylate (1 g, 3 mmol), triethylborane (1 M, 1.17 mL, CAS #97-94-9), Pd(OAc)₂ (13.1 mg, 58.4 μmol), bis(1-adamantyl)-butyl-phosphane (52.3 mg, 146 μmol) and K₃PO₄ (1.24 g, 5.84 mmol) in toluene (5 mL) and H₂O (0.5 mL) was degassed and purged with N₂ 3 times. Then the mixture was stirred at 100° C. for 4 hrs under N₂ atmosphere. On completion, the mixture was diluted with water (10 mL) and extracted with dichloromethane (2×20 mL). The combined organic layers were washed with brine (40 mL) and dried over anhydrous sodium sulfate, filtered and the filtrate was concentrated in vacuo to give the crude residue. The crude residue was purified by prep-HPLC (0.1% FA condition) to give the title compound (530 mg, 62% yield) as a yellow oil. LC-MS (ESI⁺) m/z 292.2 (M−55)⁺.

### Step 3—1-(3-Ethylpyridin-2-yl)piperazine

To a solution of tert-butyl 4-(3-ethylpyridin-2-yl)piperazine-1-carboxylate (530 mg, 1.82 mmol) in DCM (5 mL) was added TFA (1.54 g, 13.4 mmol, 1 mL). The mixture was then stirred at 25° C. for 2 hrs. On completion, the mixture was concentrated in vacuo to give the title compound (500 mg, TFA) as yellow oil. LC-MS (ESI⁺) m/z 192.9 (M+H)⁺.

### 2-(Piperazin-1-yl)nicotinonitrile (Intermediate S)

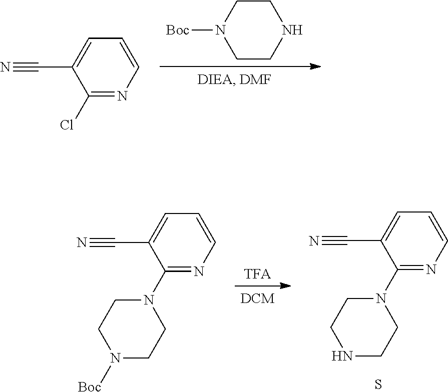

### Step 1—tert-butyl 4-(3-cyanopyridin-2-yl)piperazine-1-carboxylate

To a solution of 2-chloronicotinonitrile (2 g, 14 mmol, CAS #6602-54-6) tert-butyl piperazine-1-carboxylate (2.69 g, 14.4 mmol, CAS #57260-71-6) in ACN (20 mL) was added DIEA (9.33 g, 72.1 mmol, 12.5 mL). The mixture was then stirred at 80° C. for 16 hrs. On completion, the mixture was concentrated in vacuo to give the crude residue. The crude residue was purified by prep-HPLC (0.1% FA condition) to give the title compound (2.5 g, 60% yield) as a brown solid. LC-MS (ESI⁺) m/z 233.1 (M−55)⁺.

### Step 2—2-(Piperazin-1-yl)nicotinonitrile

To a solution of tert-butyl 4-(3-cyanopyridin-2-yl)piperazine-1-carboxylate (2.5 g, 8.7 mmol) in DCM (20 mL) was added TFA (3.07 g, 26.9 mmol, 2 mL). The mixture was then stirred at 25° C. for 12 hrs. On completion, the mixture was concentrated in vacuo to give the title compound (2 g) as brown oil. LC-MS (ESI⁺) m/z 189.0 (M+H)⁺.

### 1-(2-(Trifluoromethyl)phenyl)piperazine (CAS #63854-31-9) (Intermediate T)

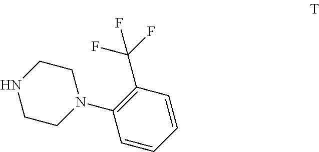 [SMILES: FC(F)(F)c1ccccc1N1CCNCC1]

### 1-(3-(2-Methoxyethoxy)pyridin-2-yl)piperazine (Intermediate U)

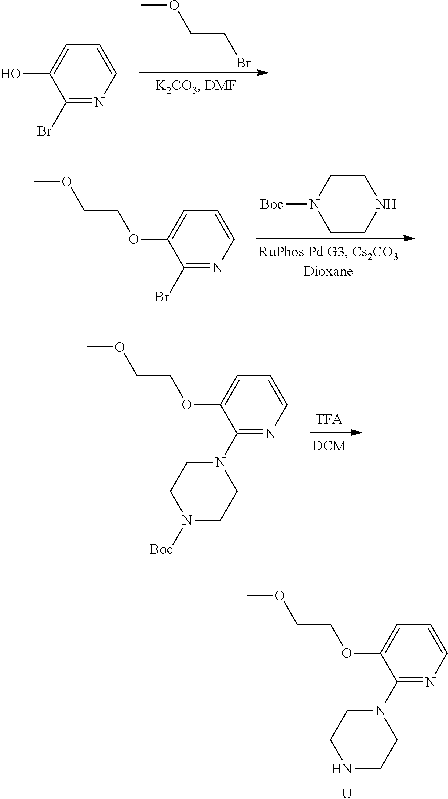

### Step 1—2-Bromo-3-(2-methoxyethoxy)pyridine

To a solution of 2-bromopyridin-3-ol (1 g, 6 mmol, CAS #6602-32-0) and 1-bromo-2-methoxyethane (798 mg, 5.75 mmol, 540 μL, CAS #6482-24-2) in DMF (5 mL) was added K₂CO₃ (2.38 g, 17.2 mmol). Then the mixture was then stirred at 80° C. for 12 hrs. On completion, the mixture was diluted with water (10 mL) and extracted with dichloromethane (2×20 mL). The combined organic layers were washed with brine (40 mL) and dried over anhydrous sodium sulfate, filtered and the filtrate was concentrated in vacuo to give the title compound (1 g, 75% yield) as yellow oil. LC-MS (ESI⁺) m/z 233.1 (M+H)⁺.

### Step 2—Tert-butyl 4-(3-(2-methoxyethoxy)pyridin-2-yl)piperazine-1-carboxylate

A mixture of 2-bromo-3-(2-methoxyethoxy)pyridine (250 mg, 1.08 mmol), tert-butyl piperazine-1-carboxylate (200 mg, 1.08 mmol, CAS #57260-71-6), RuPhos Pd G3 (90.10 mg, 107 μmol), and Cs₂CO₃ (701 mg, 2.15 mmol) in dioxane (5 mL) was degassed and purged with N₂ three times. Then the mixture was stirred at 100° C. for 4 hrs under N₂ atmosphere. On completion, the mixture was diluted with water (10 mL) and extracted with dichloromethane (2×20 mL). The combined organic layers were washed with brine (40 mL) and dried over anhydrous sodium sulfate, filtered and the filtrate was concentrated in vacuo to give the crude residue. The crude residue was purified by prep-HPLC (0.1% FA condition) to give the title compound (300 mg, 83% yield) as a yellow oil. LC-MS (ESI⁺) m/z 338.0 (M+H)⁺.

### Step 3—1-(3-(2-Methoxyethoxy)pyridin-2-yl)piperazine

To a solution of tert-butyl 4-(3-(2-methoxyethoxy)pyridin-2-yl)piperazine-1-carboxylate (300 mg, 889 μmol) in DCM (10 mL) was added TFA (1.54 g, 13.4 mmol, 1 mL). The mixture was then stirred at 25° C. for 1 hr. On completion, the mixture was concentrated in vacuo to give the title compound (300 mg) as yellow oil. LC-MS (ESI⁺) m/z 238.0 (M+H)⁺.

### 2-Methyl-3-(piperazin-1-yl)quinoxaline (Intermediate V), 4-fluoro-1-isopropyl-2-methyl-6-(piperazin-1-yl)-1H-benzo[d]imidazole (Intermediate W), 1,5-dimethyl-6-(piperazin-1-yl)-1H-indazole (Intermediate X), 1-(3-methoxynaphthalen-2-yl)piperazine (Intermediate Y), 1-(piperazin-1-yl)-2,7-naphthyridine (Intermediate BA), 4-methoxy-3-(piperazin-1-yl)benzonitrile (Intermediate BB), 1-(3,5-difluoro-2-methoxyphenyl)piperazine (Intermediate BC), 1-(3-(difluoromethoxy)pyridin-2-yl)piperazine (Intermediate BD), 1-(6-methoxypyridin-2-yl)piperazine (Intermediate BE), methyl 5-methoxy-6-(piperazin-1-yl)picolinate (Intermediate BF), 1-(3-fluoropyridin-2-yl)piperazine (Intermediate BG), 1-(piperazin-1-yl)-2,6-naphthyridine (Intermediate BH), 1-(piperazin-1-yl)-2,6-naphthyridine (Intermediate BI)

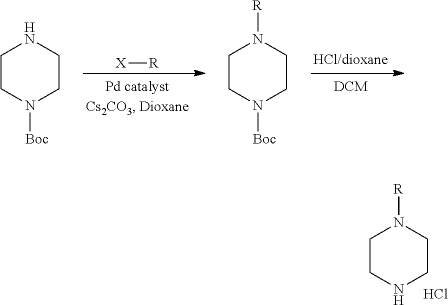

R:

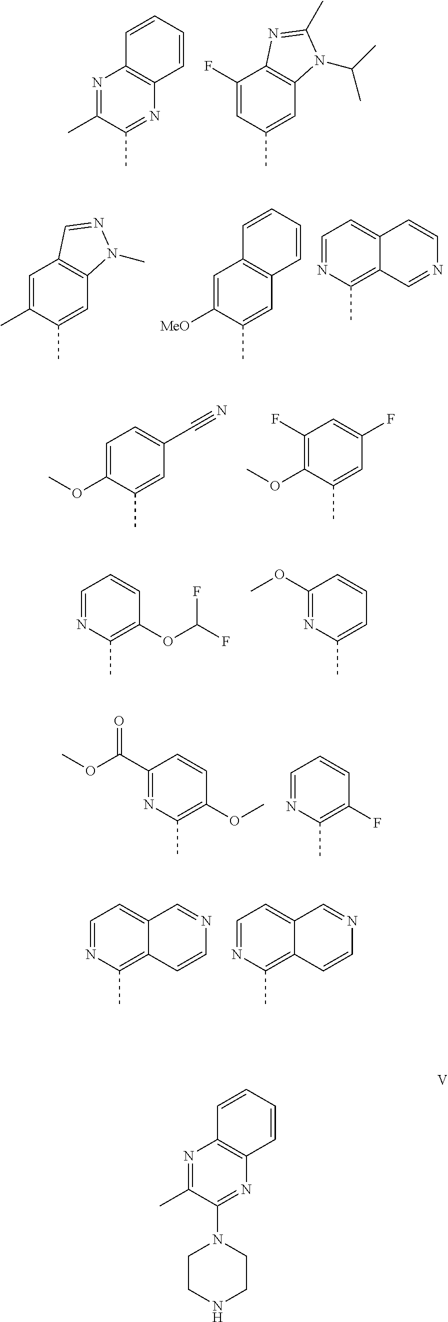

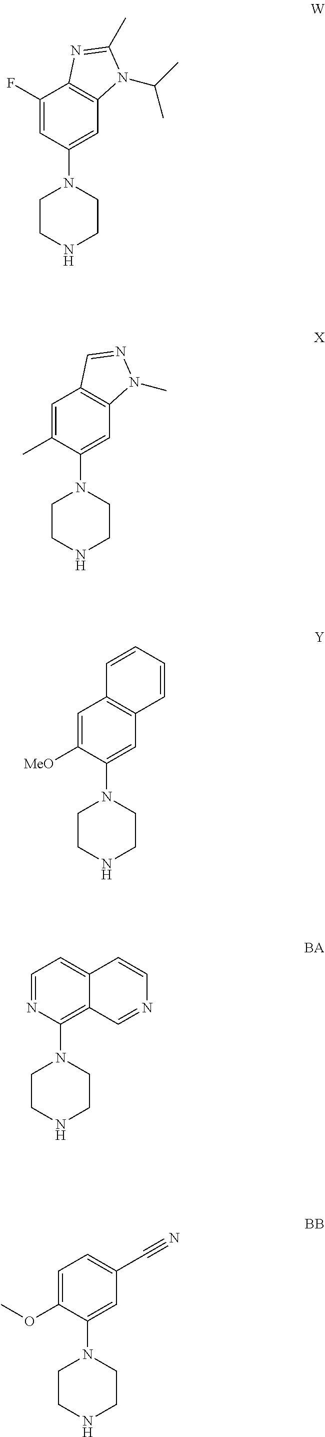

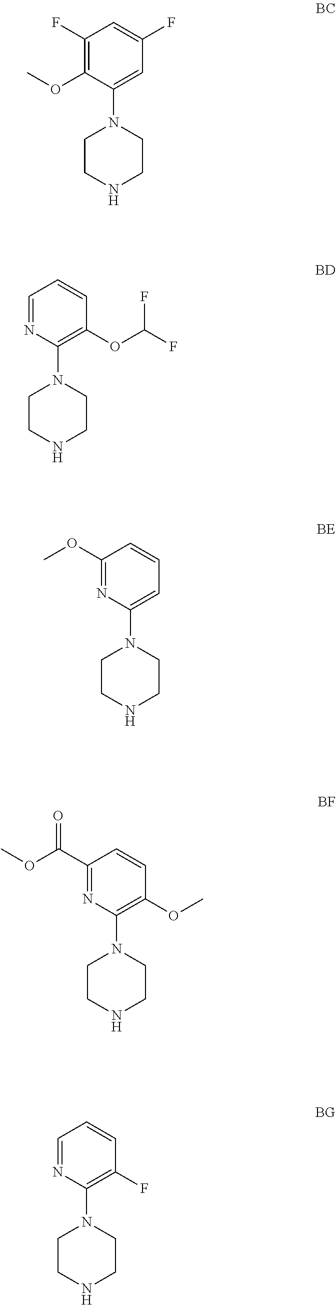

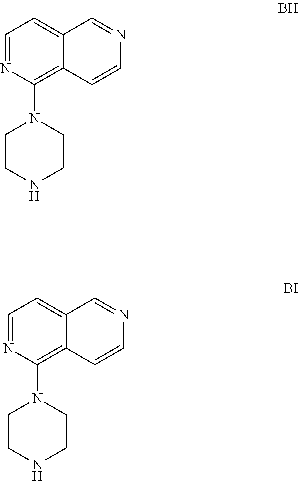 [SMILES: c1cc2c(N3CCNCC3)nccc2cn1 ; c1cc2c(N3CCNCC3)nccc2cn1]

### Step 1

To a mixture of tert-butyl piperazine-1-carboxylate (100 mg, 1.0 eq) in dioxane (5 mL) was added the corresponding reagents halide (R—X, 1.0 eq) and Cs₂CO₃ (2.5 eq). Then catalyst (SP-4-1)-[1,3-Bis[2,6-bis(1-ethylpropyl)phenyl]-4,5-dichloro-1,3-dihydro-2H-imidazol-2-ylidene]dichloro(2-methylpyridine)palladium (CAS #1612891-29-8, 0.1 eq) was added to the mixture at 25° C. under N₂ and the mixture was stirred at 100° C. under N₂ for 12 hrs. On completion, the reaction mixture was cooled to rt, diluted with H₂O (5 mL) and extracted with EtOAc (10 mL×2). The combined organic layers were washed with sat. NaCl (10 mL), dried over Na₂SO₄, filtered and concentrated under reduced pressure to give the title compounds.

### Step 2

To a mixture of the corresponding Boc protected amine (0.1, 1.0 eq) in DCM (2 mL) was added HCl/Dioxane (4N, 4 mL) at 25° C. The mixture was stirred at 25° C. for 4 hrs. On completion, the reaction mixture was concentrated under reduced pressure to give the title compounds.

### Methyl 6-(1-(3-(1H-1,2,3-triazol-1-yl)propanoyl)-1,2,5,6-tetrahydropyridin-3-yl)-4-chloro-7-fluoro-1H-indole-2-carboxylate (Intermediate Z)

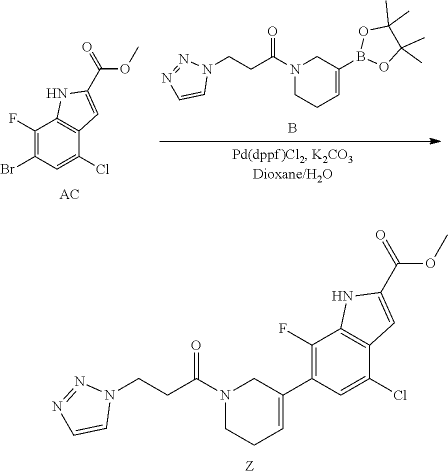

To a solution of 1-(5-(4,4,5,5-tetramethyl-1,3,2-dioxaborolan-2-yl)-3,6-dihydropyridin-1(2H)-yl)-3-(1H-1,2,3-triazol-1-yl)propan-1-one (700 mg, 2.11 mmol, Intermediate B), methyl 6-bromo-4-chloro-7-fluoro-1H-indole-2-carboxylate (710 mg, 2.32 mmol, Intermediate AC), Pd(dppf)Cl₂ (154 mg, 211 μmol), and K₂CO₃ (874 mg, 6.32 mmol) in dioxane (6 mL) and H₂O (1.5 mL) was degassed and purged with N₂ three times. Then the mixture was stirred at 80° C. for 2 hrs under N₂ atmosphere. On completion, the reaction mixture was concentrated under reduced pressure to give a residue. The residue was purified by column chromatography (SiO₂, Petroleum ether/Ethyl acetate=5/1 to 0/1, DCM:MeOH=10:1) to give the title compound (400 mg, 41% yield) as a yellow solid. LC-MS (ESI⁺) m/z 432.1 (M+H)⁺.

### 6-(1-(3-(1H-1,2,3-triazol-1-yl)propanoyl)-1,2,5,6-tetrahydropyridin-3-yl)-7-fluoro-4-(2-fluorophenyl)-1H-indole-2-carboxylic acid (Intermediate AA)

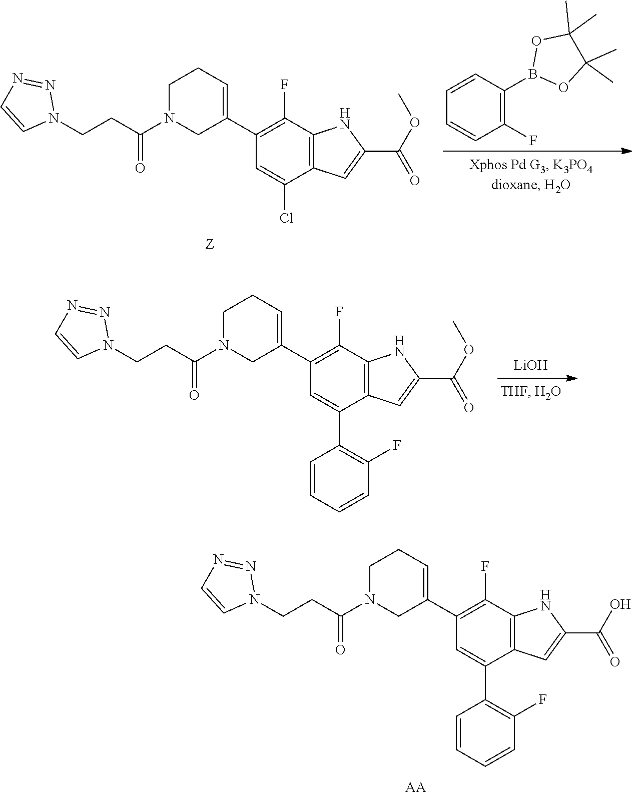

### Step 1—Methyl 6-(1-(3-(1H-1,2,3-triazol-1-yl)propanoyl)-1,2,5,6-tetrahydropyridin-3-yl)-7-fluoro-4-(2-fluorophenyl)-1H-indole-2-carboxylate

A mixture of methyl 6-(1-(3-(1H-1,2,3-triazol-1-yl)propanoyl)-1,2,5,6-tetrahydropyridin-3-yl)-4-chloro-7-fluoro-1H-indole-2-carboxylate (150 mg, 347 μmol, Intermediate Z), 2-(2-fluorophenyl)-4,4,5,5-tetramethyl-1,3,2-dioxaborolane (92.6 mg, 416 μmol, CAS #876062-39-4), XPhos Pd G3 (29.4 mg, 34.7 μmol), and K₃PO₄ (221 mg, 1.04 mmol) in dioxane (3 mL) and H₂O (0.5 mL) was degassed and purged with N₂ three times. Then the mixture was stirred at 80° C. for 2 hrs under N₂ atmosphere. On completion, the mixture was diluted with water (10 mL) and extracted with ethyl acetate (50 mL×3), dried over sodium sulfate, filtered and concentrated under reduced pressure. The residue was purified by prep-HPLC (FA condition) to give the title compound (129 mg, 73% yield) as a white solid. LC-MS (ESI⁺) m/z 492.1 (M+H)⁺.

### Step 2—6-(1-(3-(1H-1,2,3-triazol-1-yl)propanoyl)-1,2,5,6-tetrahydropyridin-3-yl)-7-fluoro-4-(2-fluorophenyl)-1H-indole-2-carboxylic acid

To a solution of methyl 7-fluoro-4-(2-fluorophenyl)-6-[1-[3-(triazol-1-yl)propanoyl]-3,6-dihydro-2H-pyridin-5-yl]-1H-indole-2-carboxylate (129 mg, 262 μmol) in THF (4 mL), H₂O (1 mL) and MeOH (1 mL) was added LiOH·H₂O (110 mg, 2.62 mmol). The mixture was then stirred at 25° C. for 2 hrs. On completion, the reaction mixture was adjust pH to 5-6 with 2M HCl, then extracted with DCM 30 mL (10 mL×3). The combined organic layers were washed with brine (50 mL×2), dried over Na₂SO₄, filtered and concentrated under reduced pressure to give the title compound (65 mg) as a yellow solid. LC-MS (ESI⁺) m/z 478.2 (M+H)⁺.

### 6-(1-(3-(1H-1,2,3-triazol-1-yl)propanoyl)-1,2,5,6-tetrahydropyridin-3-yl)-7-fluoro-4-(o-tolyl)-1H-indole-2-carboxylic acid (Intermediate AB)

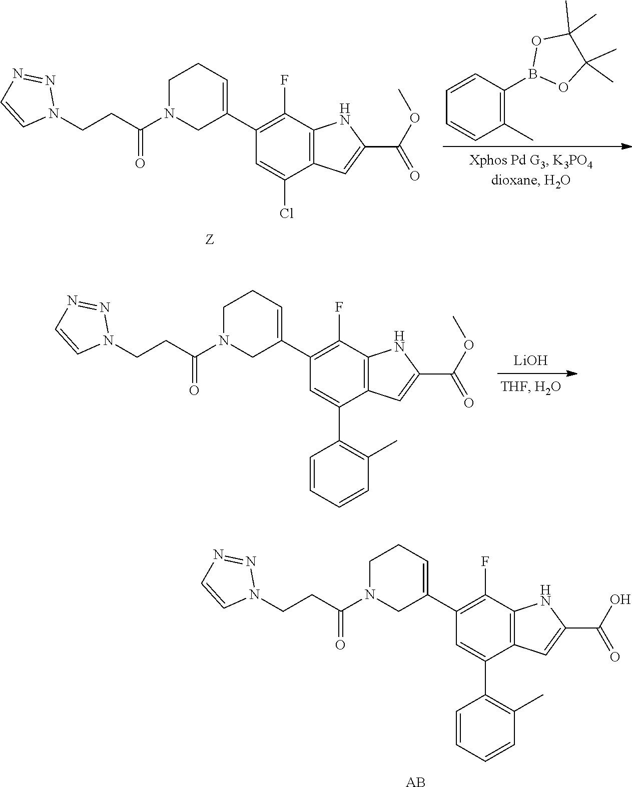

### Step 1—Methyl 6-(1-(3-(1H-1,2,3-triazol-1-yl)propanoyl)-1,2,5,6-tetrahydropyridin-3-yl)-7-fluoro-4-(o-tolyl)-1H-indole-2-carboxylate

A mixture of methyl 6-(1-(3-(1H-1,2,3-triazol-1-yl)propanoyl)-1,2,5,6-tetrahydropyridin-3-yl)-4-chloro-7-fluoro-1H-indole-2-carboxylate (150 mg, 347 μmol, Intermediate Z), 4,4,5,5-tetramethyl-2-(o-tolyl)-1,3,2-dioxaborolane (91 mg, 416 μmol, CAS #195062-59-0), XPhos Pd G3 (29.40 mg, 34.73 μmol), and K₃PO₄ (221.19 mg, 1.04 mmol) in dioxane (8 mL) and H₂O (1 mL) was degassed and purged with N₂ three times. Then the mixture was stirred at 80° C. for 2 hrs under N₂ atmosphere. On completion, the mixture was diluted with water (10 mL) and extracted with ethyl acetate (50 mL×3), dried over sodium sulfate, filtered and concentrated under reduced pressure. The residue was purified by prep-HPLC (FA condition) to give the title compound (100 mg, 48% yield) as a white solid. LC-MS (ESI⁺) m/z 488.2 (M+H)⁺.

### Step 2—6-(1-(3-(1H-1,2,3-triazol-1-yl)propanoyl)-1,2,5,6-tetrahydropyridin-3-yl)-7-fluoro-4-(o-tolyl)-1H-indole-2-carboxylic acid

To a solution of methyl 7-fluoro-4-(o-tolyl)-6-[1-[3-(triazol-1-yl)propanoyl]-3,6-dihydro-2H-pyridin-5-yl]-1H-indole-2-carboxylate (93 mg, 190.7 μmol) in H₂O (1 mL) and THF (4 mL) was added LiOH·H₂O (80.1 mg, 1.91 mmol). The mixture was then stirred at 25° C. for 2 hrs. On completion, the reaction mixture was adjust pH to 5-6 by 2M HCl, then extracted with DCM (10 mL×3). The combined organic layers were washed with brine (50 mL×2), dried over Na₂SO₄, filtered and concentrated under reduced pressure to give the title compound (45 mg) as a yellow solid. LC-MS (ESI⁺) m/z 474.2 (M+H)⁺.

### Methyl 6-bromo-4-chloro-7-fluoro-1H-indole-2-carboxylate (Intermediate AC)

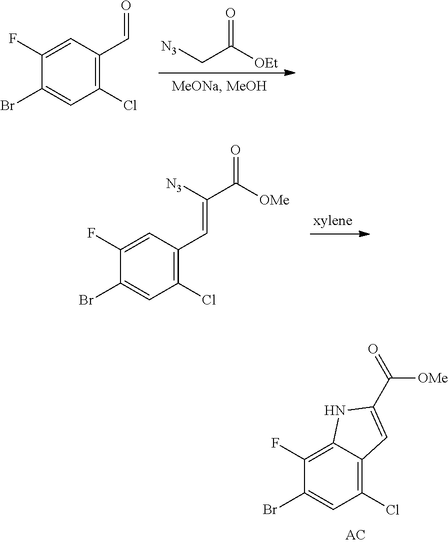

### Step 1—Methyl 2-azido-3-(4-bromo-2-chloro-5-fluorophenyl) acrylate

A solution of 4-bromo-2-chloro-5-fluorobenzaldehyde (18 g, 76 mmol) and ethyl 2-azidoacetate (39.1 g, 303 mmol) in MeOH (250 mL) was added to a mixture of NaOMe (5.4 M, 56.1 mL) in MeOH (250 mL) dropwise at −10° C. The mixture was warmed up to 20° C. and stirred for 12 hrs. On completion, the reaction mixture was poured into NH₄Cl (600 mL) at 0° C., then filtered and the solid was dried in vacuo. The residue was purified by column chromatography (SiO₂, Petroleum ether. Ethyl acetate=100/1 to 4/1) to give the title compound (17 g, 67% yield) as a white solid. ¹H NMR (400 MHz, DMSO-d₆) δ=8.21 (d, J=10.4 Hz, 1H), 8.06 (d, J=6.8 Hz, 1H), 7.01 (s, 1H), 3.94 (s, 3H).

### Step 2—Methyl 6-bromo-4-chloro-7-fluoro-1H-indole-2-carboxylate

Methyl 2-azido-3-(4-bromo-2-chloro-5-fluorophenyl) acrylate (15 g, 44.84 mmol) was added to xylene (500 mL) and the reaction was refluxed for 3 hrs. On completion, the reaction mixture was cooled to rt, filtered and the filter cake was dried in vacuo to give the title compound (11 g, 80% yield) as a white solid. ¹H NMR (400 MHz, DMSO-d₆) δ=13.09 (s, 1H), 7.44 (d, J=4.4 Hz, 1H), 7.16 (s, 1H), 3.90 (s, 3H).

### 6-Bromo-4-chloro-7-fluoro-1H-indole-2-carboxylic acid (Intermediate AD)

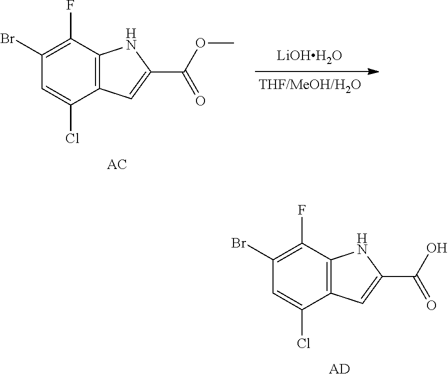

A mixture of methyl 6-bromo-4-chloro-7-fluoro-1H-indole-2-carboxylate (1 g, 3.26 mmol, Intermediate AC) and LiOH H₂O (1.37 g, 32.6 mmol) in THF (3 mL), MeOH (3 mL), and H₂O (3 mL) at 25° C., and then the mixture was stirred at 40° C. for 4 hrs. On completion, to the reaction mixture was added HCl (2 N) until the pH=3, then the mixture was diluted with water (30 mL) and extracted with ethyl acetate (3×50 mL). The combined organic layers were dried over anhydrous sodium sulfate, filtered and the filtrate was concentrated in vacuo to give the compound (1 g) as yellow solid. LC-MS (ESI⁺) m/z 291.5 (M−H)⁻.

### 3-(1H-pyrazol-1-yl)-1-(5-(4,4,5,5-tetramethyl-1,3,2-dioxaborolan-2-yl)-3,6-dihydropyridin-1(2H)-yl)propan-1-one (Intermediate AE)

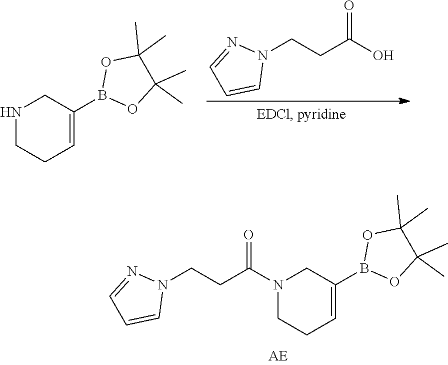

To a solution of 3-(1H-pyrazol-1-yl)propanoic acid (25 g, 178 mmol, CAS #89532-73-0) in anhydrous pyridine (200 mL) was added EDCI (41.0 g, 214 mmol). Then 5-(4,4,5,5-tetramethyl-1,3,2-dioxaborolan-2-yl)-1,2,3,6-tetrahydropyridine (48.2 g, 196 mmol, HCl, synthesized via Step 1 of Intermediate E) was added, and the reaction was stirred at 20° C. for 5 h under nitrogen atmosphere. On completion, the mixture was concentrated to remove pyridine and give a crude residue. The residue was purified by column chromatography (SiO₂, dichloromethan:ethyl acetate=10/1 to 1/1) to give the title compound (43 g, 73% yield) as a white solid. LC-MS (ESI⁺) m/z 332.0 (M+H)⁺. ¹H NMR (400 MHz, DMSO-d6) δ=7.69 (s, 1H), 7.49-7.29 (m, 1H), 6.66-6.40 (m, 1H), 6.19 (s, 1H), 4.32 (br t, J=6.8 Hz, 2H), 4.00-3.85 (m, 2H), 3.55-3.38 (m, 2H), 2.88 (q, J=6.8 Hz, 2H), 2.25-2.05 (m, 2H), 1.21 (s, 12H).

### 6-(1-(3-(1H-pyrazol-1-yl)propanoyl)-1,2,5,6-tetrahydropyridin-3-yl)-4-chloro-7-fluoro-1H-indole-2-carboxylic acid (Intermediate AF)

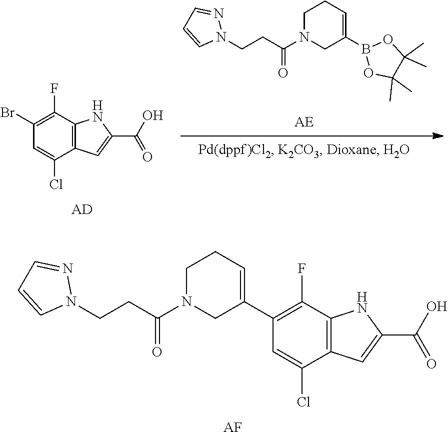

A mixture of 6-bromo-4-chloro-7-fluoro-1H-indole-2-carboxylic acid (200 mg, 608 μmol, HCl, Intermediate AD), 3-pyrazol-1-yl-1-[5-(4,4,5,5-tetramethyl-1,3,2-dioxaborolan-2-yl)-3,6-dihydro-2H-pyridin-1-yl]propan-1-one (201 mg, 608 μmol, Intermediate AE), Pd(dppf)Cl₂ (44.5 mg, 60.8 μmol), and K₂CO₃ (252 mg, 1.82 mmol) in dioxane (5 mL) and H₂O (1 mL) was degassed and purged with N₂ for 3 times at 25° C. Then the mixture was stirred at 80° C. for 1 hr under N₂ atmosphere. The reaction mixture was concentrated under reduced pressure to remove solvent. The crude product was triturated with ethyl acetate (0.5 mL) and Petroleum ether (5 mL) at 25° C. for 10 min, then the mixture was filtered to give the compound (170 mg, 67% yield) as a brown solid. LC-MS (ESI⁺) m/z 415.1 (M−H)⁻.

### 6-(1-(3-(1H-pyrazol-1-yl)propanoyl)-1,2,5,6-tetrahydropyridin-3-yl)-7-fluoro-4-(2-methoxyphenyl)-1H-indole-2-carboxylic acid (Intermediate AG)

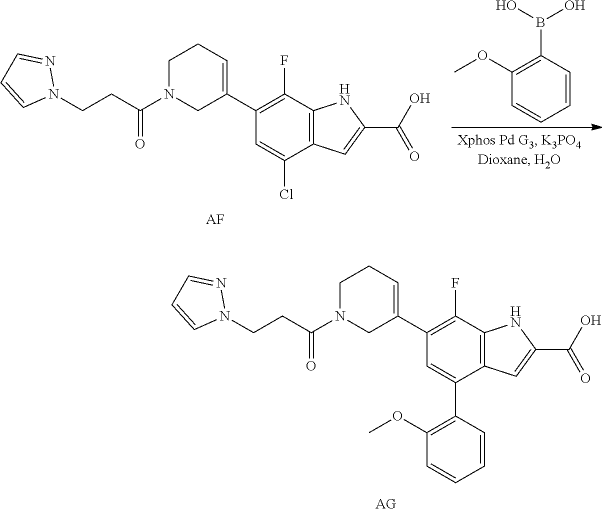 [SMILES: O=C(O)c1cc2c(Cl)cc(C3=CCCN(C(=O)CCn4cccn4)C3)c(F)c2[nH]1 ; COc1ccccc1B(O)O ; "AF" ; COc1ccccc1-c1cc(C2=CCCN(C(=O)CCn3cccn3)C2)c(F)c2[nH]c(C(=O)O)cc12 ; "AG"]

A mixture of 4-chloro-7-fluoro-6-[1-(3-pyrazol-1-ylpropanoyl)-3,6-dihydro-2H-pyridin-5-yl]-1H-indole-2-carboxylic acid (70 mg, 168 μmol, Intermediate AF), 2-methoxyphenyl)boronic acid (25.5 mg, 168 μmol, CAS #5720-06-9), XPhos Pd G₃ (14.2 mg, 16.8 μmol), and K₃PO₄ (107 mg, 504 μmol) in dioxane (2 mL) and H₂O (0.4 mL) was degassed and purged with N₂ 3 times at 25° C. Then the mixture was stirred at 80° C. for 1 hr under N₂ atmosphere. The reaction mixture was concentrated under reduced pressure to remove solvent. The crude residue was purified by reversed-phase HPLC (0.1% FA condition) to give the title compound (40 mg, 49% yield) as a brown solid. LC-MS (ESI⁺) m/z 489.1 (M+H)⁺.

### 6-(1-(3-(1H-1,2,3-triazol-1-yl)propanoyl)piperidin-3-yl)-7-fluoro-4-(2-methoxyphenyl)-1H-indole-2-carboxylic acid (Intermediate AH)

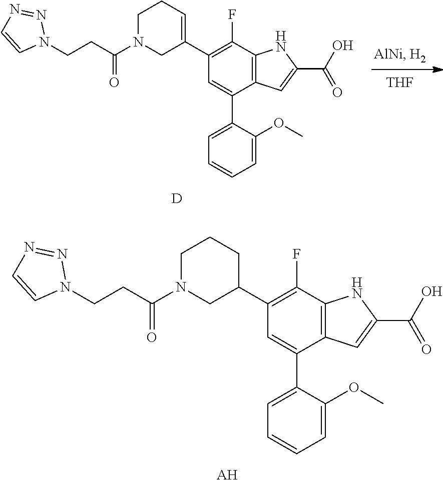

A mixture of 7-fluoro-4-(2-methoxyphenyl)-6-[1-[3-(triazol-1-yl)propanoyl]-3,6-dihydro 2H-pyridin-5-yl]-1H-indole-2-carboxylic acid (200 mg, 408.5 μmol, Intermediate D) in THF (2 mL) was degassed and purged with N₂ three times at 25° C. Next, alumane nickel (700.1 mg, 8.1 mmol) was added, then the mixture was purged with H₂ three times. Then the mixture was stirred at 50° C. for 24 hrs under H₂ atmosphere (50 PSI). On completion, the reaction mixture was concentrated under reduced pressure to remove THF, filtered and concentrated under reduced pressure to give a residue. The residue was purified by prep-HPLC (column: CD01-Phenomenex luna C18 150*25*10 um;mobile phase: [water(FA)-ACN]; gradient:29%-599% B over 10 min) to give the title compound (50 mg, 25% yield) as a white solid. LC-MS (ESI⁺) m/z 492.3 (M+H)⁺.

### (4-Chloro-7-fluoro-6-(1,2,5,6-tetrahydropyridin-3-yl)benzo[b]thiophen-2-yl)(4-(3-methoxypyridin-2-yl)piperazin-1-yl)methanone (Intermediate AJ)

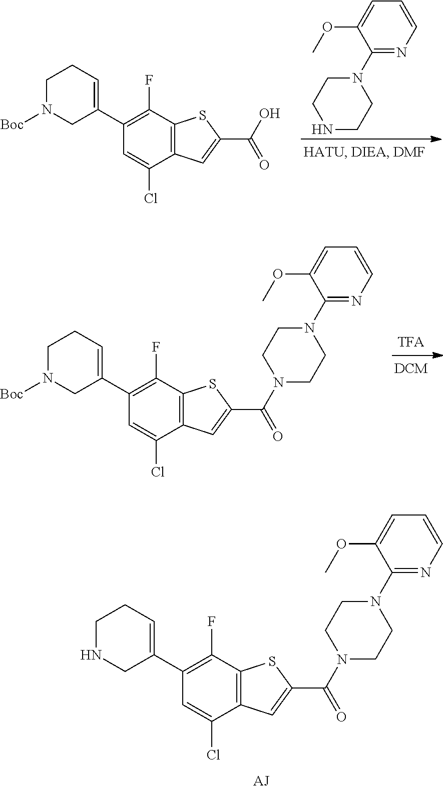

### Step 1—Tert-butyl 3-(4-chloro-7-fluoro-2-(4-(3-methoxypyridin-2-yl)piperazine-1-carbonyl)benzo[b]thiophen-6-yl)-5,6-dihydropyridine-1(2H)-carboxylate

To a solution of 6-(1-(tert-butoxycarbonyl)-1,2,5,6-tetrahydropyridin-3-yl)-4-chloro-7-fluorobenzo[b]thiophene-2-carboxylic acid (2 g, 4.86 mmol, synthesized via Steps 1-4 of Intermediate K) in DMF (20 mL) was added HATU (2.22 g, 5.83 mmol) and DIEA (3.14 g, 24.3 mmol, 4.23 mL) and 1-(3-methoxypyridin-2-yl)piperazine (1.79 g, 5.83 mmol, CAS #80827-67-4). The mixture was then stirred at 25° C. for 2 hrs. On completion, the crude product was triturated with H₂O at 0° C. for 15 min to give the title compound (2.5 g) as a white solid. LC-MS (ESI⁺) m/z 587.2 (M+H)⁺.

### Step 2—(4-Chloro-7-fluoro-6-(1,2,5,6-tetrahydropyridin-3-yl)benzo[b]thiophen-2-yl)(4-(3-methoxypyridin-2-yl)piperazin-1-yl)methanone

To a solution of tert-butyl 3-(4-chloro-7-fluoro-2-(4-(3-methoxypyridin-2-yl)piperazine-1-carbonyl)benzo[b]thiophen-6-yl)-5,6-dihydropyridine-1(2H)-carboxylate (2 g, 3 mmol) in DCM (20 mL) was added TFA (1.94 g, 17.0 mmol, 1.27 mL). Then the mixture was stirred at 25° C. for 2 hrs. On completion, the mixture was concentrated in vacuo to give the title compound (400 mg) as a white solid. LC-MS (ESI⁺) m/z 487.2 (M+H)⁺.

### 1-(3-(4-Chloro-7-fluoro-2-(4-(3-methoxypyridin-2-yl)piperazine-1-carbonyl)benzo[b]thiophen-6-yl)-5,6-dihydropyridin-1(2H)-yl)-3-(1H-1,2,3-triazol-1-yl)propan-1-one (Intermediate AK)

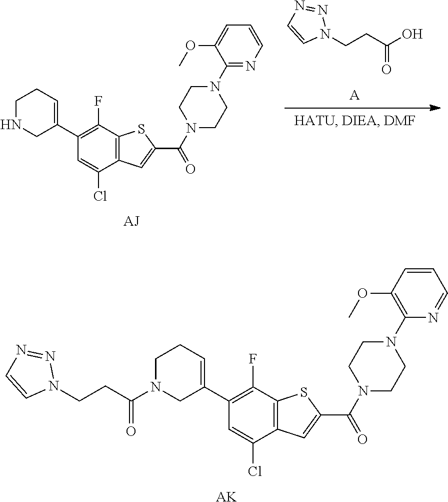

To a solution of 3-(triazol-1-yl)propanoic acid (117 mg, 834 μmol, Intermediate A) in DMF (5 mL) was added HATU (316 mg, 832 μmol), DIEA (108 mg, 832 μmol) and (4-chloro-7-fluoro-6-(1,2,5,6-tetrahydropyridin-3-yl)benzo[b]thiophen-2-yl)(4-(3-methoxypyridin-2-yl)piperazin-1-yl)methanone (500 mg, 832 μmol, TFA, Intermediate AJ). The mixture was then stirred at 25° C. for 2 hrs. On completion, the crude product was triturated with H₂O at 0° C. for 15 min to give the title compound (2.5 g) as a white solid. LC-MS (ESI⁺) m/z 462.1 (M+H)⁺.

### 1-(2-Methoxypyridin-3-yl)piperazine (Intermediate AL)

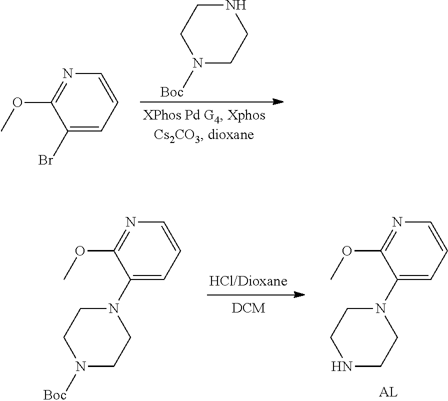

### Step 1—Tert-butyl 4-(2-methoxypyridin-3-yl)piperazine-1-carboxylate

To a solution of 3-bromo-2-methoxypyridine (1 g, 5 mmol, CAS #13472-59-8), tert-butyl piperazine-1-carboxylate (1.49 g, 7.98 mmol, CAS #57260-71-6) in dioxane (10 mL) was added Cs₂CO₃ (6.93 g, 21.2 mmol), XPhos (304 mg, 638 μmol) and Xphos Pd G4 (457 mg, 531 μmol). Then the reaction was stirred at 80° C. for 12 hrs under nitrogen atmosphere. On completion, the reaction mixture was filtered and the filtrate was concentrated in vacuo to give the crude residue. The crude residue was purified by column chromatography (SiO₂, Petroleum ether/Ethyl acetate=1/0 to 10/1) to give the title compound (1 g, 62% yield) as a yellow solid. LC-MS (ESI⁺) m/z 294.2 (M+H)⁺.

### Step 2—1-(2-Methoxypyridin-3-yl)piperazine

To a solution of tert-butyl 4-(2-methoxypyridin-3-yl)piperazine-1-carboxylate (1 g, 3 mmol) in DCM (10 mL) was added HCl/dioxane (8 M, 2 mL). The mixture was then stirred at 25° C. for 1 hr. On completion, the reaction mixture was filtered and the filter cake was dried in vacuo to give the title compound (500 mg, HCl) as a white solid. LC-MS (ESI⁺) m/z 194.1 (M+H)⁺.

### 1-(3-Methoxypyridin-4-yl)piperazine (Intermediate AM)

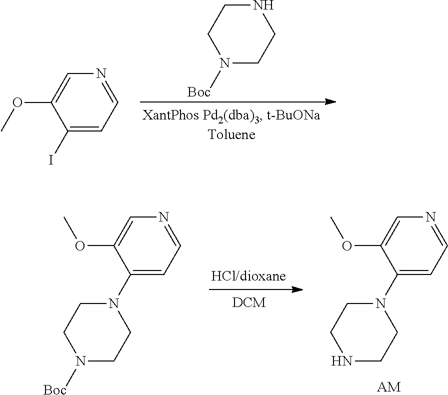

### Step 1—Tert-butyl 4-(3-methoxypyridin-4-yl)piperazine-1-carboxylate

A mixture of 4-iodo-3-methoxy-pyridine (300 mg, 1.28 mmol, CAS #1331850-50-0), tert-butyl piperazine-1-carboxylate (237 mg, 1.28 mmol), t-BuONa (613 mg, 6.38 mmol), Xantphos (73.8 mg, 127 μmol) and Pd₂(dba)₃ (116 mg, 127 μmol) in toluene (5 mL) was degassed and purged with N₂ three times. Then the mixture was stirred at 100° C. for 2 hrs under N₂ atmosphere. On completion, the reaction mixture was concentrated under reduced pressure to give a residue. The residue was purified by column chromatography (SiO₂, Petroleum ether/Ethyl acetate=1/0 to 0/1) to give the title compound (300 mg, 47% yield) as a yellow solid. LC-MS (ESI⁺) m/z 294.5 (M+H)⁺.

### Step 2—1-(3-Methoxypyridin-4-yl)piperazine

To a solution of tert-butyl 4-(3-methoxypyridin-4-yl)piperazine-1-carboxylate (300 mg, 1 mmol) in DCM (8 mL) was added HCl/dioxane (8 M, 127 μL). The mixture was then stirred at 25° C. for 2 hrs. On completion, the reaction mixture was filtered and the filter cake was dried in vacuo to give the title compound (200 mg) as a light yellow solid. ¹H NMR (400 MHz, DMSO-d₆) δ=9.46 (br dd, J=1.6, 2.8 Hz, 1H), 8.30-8.25 (m, 2H), 7.31 (d, J=6.4 Hz, 1H), 3.96 (s, 3H), 3.90-3.84 (m, 4H), 3.24 (br s, 4H).

### Tert-butyl (R)-3-(4-chloro-2-(dimethylcarbamoyl)-7-fluoro-1H-indol-6-yl)piperidine-1-carboxylate (Intermediate AN) & tert-butyl (S)-3-(4-chloro-2-(dimethylcarbamoyl)-7-fluoro-1H-indol-6-yl)piperidine-1-carboxylate (Intermediate AO)

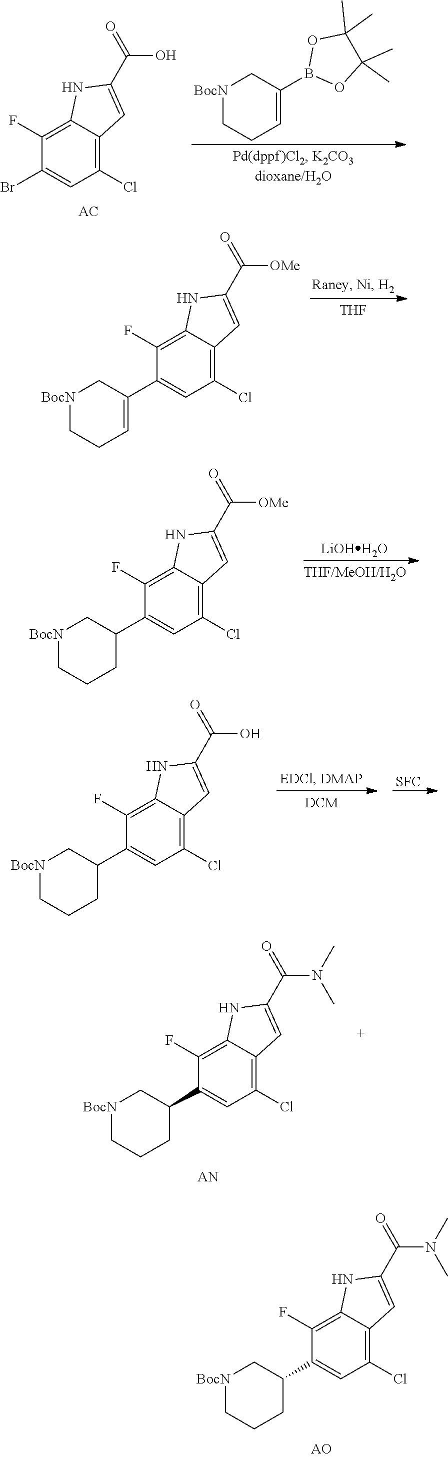

### Step 1—Methyl 6-(1-(tert-butoxycarbonyl)-1,2,5,6-tetrahydropyridin-3-yl)-4-chloro-7-fluoro-1H-indole-2-carboxylate

To a solution of methyl 6-bromo-4-chloro-7-fluoro-1H-indole-2-carboxylate (1 g, 3.26 mmol, Intermediate AC) and tert-butyl 5-(4,4,5,5-tetramethyl-1,3,2-dioxaborolan-2-yl)-3,6-dihydropyridine-1(2H)-carboxylate (1.01 g, 3.26 mmol, CAS #885693-20-9) in dioxane (10 mL) and H₂O (3 mL) was added Pd(dppf)Cl₂ (238 mg, 326 μmol) and K₂CO₃ (1.35 g, 9.79 mmol). The mixture was then stirred at 60° C. for 1 hr. On completion, the reaction mixture was diluted with water (100 mL) and extracted with DCM (100 mL×3). The combined organic layers were washed with brine (100 mL×2), dried over Na₂SO₄, filtered and concentrated under reduced pressure to give a residue. The crude product was triturated with ACN (100 mL) at 20° C. for 30 min, then filtered to give the title compound (1.2 g) as a yellow solid LC-MS (ESI⁺) m/z 353.0 (M+H-56)⁺.

### Step 2—Methyl 6-(1-(tert-butoxycarbonyl)piperidin-3-yl)-4-chloro-7-fluoro-1H-indole-2-carboxylate

To a solution of methyl 6-(1-(tert-butoxycarbonyl)-1,2,5,6-tetrahydropyridin-3-yl)-4-chloro-7-fluoro-1H-indole-2-carboxylate (560 mg, 1.37 mmol) in THF (15 mL) was added Raney-Ni (469 mg, 5.48 mmol) under N₂ atmosphere. The suspension was degassed and purged with H₂ three times. Then the mixture was stirred under H₂ (15 PSI) at 25° C. for 10 hrs. On completion, the reaction was filtered through kieselguhr very carefully, and the filtrate was concentrated in vacuo to give the title compound (490 mg) as a light yellow solid. LC-MS (ESI⁺) m/z 355.0 (M+H-56)⁺.

### Step 3—6-(1-(Tert-butoxycarbonyl)piperidin-3-yl)-4-chloro-7-fluoro-1H-indole-2-carboxylic acid

To a solution of methyl 6-(1-(tert-butoxycarbonyl)piperidin-3-yl)-4-chloro-7-fluoro-1H-indole-2-carboxylate (490 mg, 834 μmol) in THF (4 mL), H₂O (1 mL) and MeOH (1 mL) was added LiOH·H₂O (280 mg, 6.68 mmol). The mixture was then stirred at 25° C. for 12 hrs. On completion, HCl (1N) was added to the reaction mixture until the pH=7, then diluted with water (50 mL) and extracted with ethyl acetate (3×50 mL). The combined organic layers were dried over anhydrous sodium sulfate, filtered and the filtrate was concentrated in vacuo to give the title compound (435 mg) as a yellow solid. LC-MS (ESI⁺) m/z 341.0 (M+H-56)⁺.

### Step 4—Tert-butyl (R)-3-(4-chloro-2-(dimethylcarbamoyl)-7-fluoro-1H-indol-6-yl)piperidine-1-carboxylate & tert-butyl (S)-3-(4-chloro-2-(dimethylcarbamoyl)-7-fluoro-1H-indol-6-yl)piperidine-1-carboxylate

To a solution of 6-(1-(tert-butoxycarbonyl)piperidin-3-yl)-4-chloro-7-fluoro-1H-indole-2-carboxylic acid (400 mg, 1.01 mmol) in DCM (8 mL) was added EDCI (231 mg, 1.21 mmol) and DMAP (492 mg, 4.03 mmol) and the mixture was stirred at 20° C. for 1 hr. Then N-methylmethanamine hydrochloride (123 mg, 1.51 mmol) was added and the mixture was stirred at 20° C. for 11 hrs. On completion, the reaction mixture was concentrated under reduced pressure to give a residue and the crude product was triturated with water (20 mL) at 20° C. for 15 min. The residue was purified by prep-HPLC (neutral condition) and then purified by SFC (column: DAICEL CHIRALPAK AD(250 mm*30 mm, 10 um); mobile phase: [CO₂-i-PrOH]; B %: 40%, isocratic elution mode column to give the tert-butyl (R)-3-(4-chloro-2-(dimethylcarbamoyl)-7-fluoro-1H-indol-6-yl)piperidine-1-carboxylate (75 mg, 17% yield) as a white solid and tert-butyl (S)-3-(4-chloro-2-(dimethylcarbamoyl)-7-fluoro-1H-indol-6-yl)piperidine-1-carboxylate (90 mg, 21% yield) as a white solid. LC-MS (ESI⁺) m/z 368.3 (M+H-56) for both isomers. The absolute stereochemistry of the enantiomers was assigned arbitrarily.

### Methyl (R)-6-(1-(tert-butoxycarbonyl)piperidin-3-yl)-4-chloro-7-fluoro-1H-indole-2-carboxylate (Intermediate AP) and methyl (S)-6-(1-(tert-butoxycarbonyl)piperidin-3-yl)-4-chloro-7-fluoro-1H-indole-2-carboxylate (Intermediate AO)

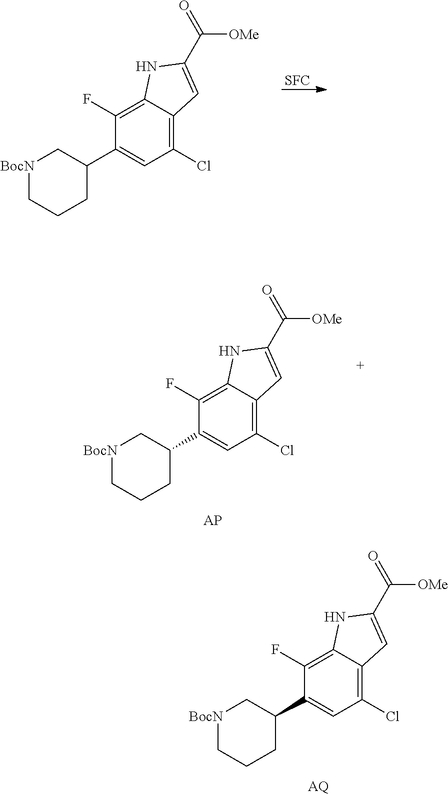

### Step 1—Methyl (R)-6-(1-(tert-butoxycarbonyl)piperidin-3-yl)-4-chloro-7-fluoro-1H-indole-2-carboxylate

Methyl 6-(1-(tert-butoxycarbonyl)piperidin-3-yl)-4-chloro-7-fluoro-1H-indole-2-carboxylate (50.0 g, 122 mmol, synthesized via Steps 1-2 of Intermediate AN) was purified by Prep-HPLC (column: DAICEL CHIRALPAK IG (250 mm*30 mm, 10 um); mobile phase: [CO₂—MeOH (0.1% NH₃H₂O)]; B %: 30%, isocratic elution mode) to give methyl (R)-6-(1-(tert-butoxycarbonyl)piperidin-3-yl)-4-chloro-7-fluoro-1H-indole-2-carboxylate (18.5 g, 74% yield) as a white solid (LC-MS (ESI⁺) m/z 355.1; ¹H NMR 400 MHz, DMSO-d₆ δ 12.8 (s, 1H), 7.17 (d, J=5.2 Hz, 1H), 7.12 (d, J=2.8 Hz, 1H), 3.96 (d, J=12.4 Hz, 2H), 3.89 (s, 3H), 3.04˜2.99 (m, 3H), 1.84˜1.73 (m, 3H), 1.49˜1.46 (m, 1H), 1.41 (s, 9H)) and methyl (S)-6-(1-(tert-butoxycarbonyl)piperidin-3-yl)-4-chloro-7-fluoro-1H-indole-2-carboxylate (20.0 g, 80.0% yield) as a white solid (LC-MS (ESI⁺) m/z 355.1; ¹H NMR 400 MHz, DMSO-d₆ δ 12.9˜12.7 (m, 1H), 7.17 (d, J=4.8 Hz, 1H), 7.12 (d, J=2.8 Hz, 1H), 3.96 (d, J=12.0 Hz, 2H), 3.89 (s, 3H), 3.04˜2.84 (m, 3H), 1.88˜1.70 (m, 3H), 1.49˜1.42 (m, 1H), 1.41 (s, 9H)). Absolute stereochemistry of the enantiomers were assigned arbitrarily.

### (R)-methyl 4-chloro-7-fluoro-6-(piperidin-3-yl)-1H-indole-2-carboxylate (Intermediate AR)

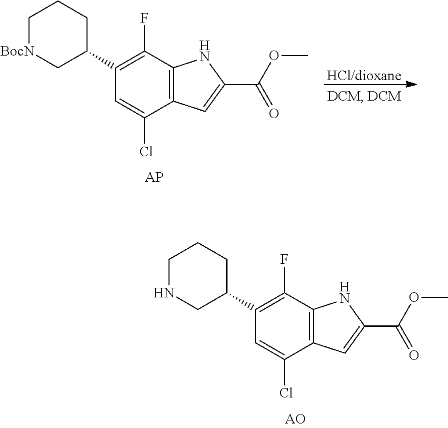

To a solution of (R)-methyl 6-(1-(tert-butoxycarbonyl)piperidin-3-yl)-4-chloro-7-fluoro-1H-indole-2-carboxylate (2 g, 4.87 mmol, Intermediate AP) in anhydrous DCM (30 mL) was added HCl/dioxane (8 M, 6 mL) at 25° C. under nitrogen atmosphere, then the reaction was stirred at 25° C. for 2 h under nitrogen atmosphere. On completion, the mixture was concentrated to give the title compound (2 g, HCl) as a white solid. LC-MS (ESI⁺) m/z 311.0 (M+H)⁺.

### (R)-6-(1-(3-(1H-1,2,3-triazol-1-yl)propanoyl)piperidin-3-yl)-4-chloro-7-fluoro-1H-indole-2-carboxylic acid (Intermediate AS)

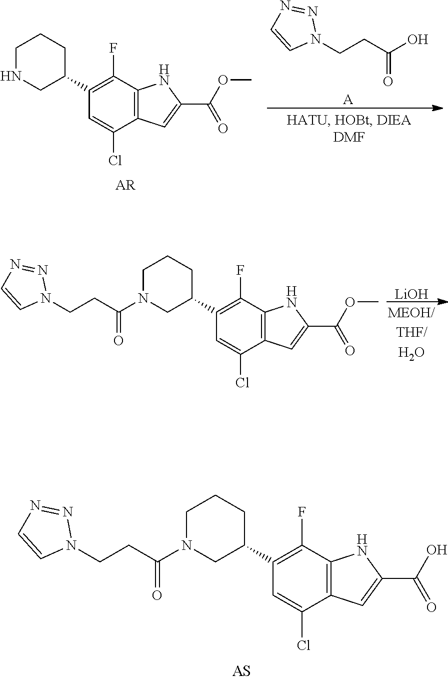

### Step 1—(R)-methyl 6-(1-(3-(1H-1,2,3-triazol-1-yl)propanoyl)piperidin-3-yl)-4-chloro-7-fluoro-1H-indole-2-carboxylate

To a solution of 3-(1H-1,2,3-triazol-1-yl)propanoic acid (812 mg, 5.76 mmol, Intermediate A) in anhydrous DMF (10 mL) was added DIEA (3.72 g, 28.8 mmol, 5.02 mL), HOBt (778 mg, 5.76 mmol) and HATU (1.64 g, 4.32 mmol) at 0° C. under nitrogen atmosphere, then (R)-methyl 4-chloro-7-fluoro-6-(piperidin-3-yl)-1H-indole-2-carboxylate (1 g, 2.88 mmol, HCl, Intermediate AR) was added and reaction was stirred at 0° C. for 1 h under nitrogen atmosphere. On completion, the reaction mixture was diluted with water (50 mL) and extracted with ethyl acetate (3×50 mL). The combined organic layers were washed with brine (100 mL) and dried over anhydrous sodium sulfate, filtered and the filtrate was concentrated in vacuo to give the title compound (1.2 g,) as a white solid. LC-MS (ESI⁺) m/z 434.0 (M+H)⁺.

### Step 2—(R)-6-(1-(3-(1H-1,2,3-triazol-1-yl)propanoyl)piperidin-3-yl)-4-chloro-7-fluoro-1H-indole-2-carboxylic acid

To a solution of (R)-methyl 6-(1-(3-(1H-1,2,3-triazol-1-yl)propanoyl)piperidin-3-yl)-4-chloro-7-fluoro-1H-indole-2-carboxylate (1 g, 2 mmol) in anhydrous THF (10 mL), MeOH (6 mL) and H₂O (6 mL) was added LiOH·H₂O (290 mg, 6.91 mmol) at 25° C. under nitrogen atmosphere. Then the reaction was stirred at 25° C. for 2 h under nitrogen atmosphere. On completion, to the reaction mixture was added HCl (1N) until the pH=4, then diluted with water (10 mL) and extracted with ethyl acetate (3×30 mL). The combined organic layers were dried over anhydrous sodium sulfate, filtered and the filtrate was concentrated in vacuo to give the title compound (1 g) as white solid. LC-MS (ESI⁺) m/z 420.1 (M+H)⁺.

### (R)-1-(3-(4-chloro-7-fluoro-2-(4-(3-methoxypyridin-2-yl)piperazine-1-carbonyl)-1H-indol-6-yl)piperidin-1-yl)-3-(1H-1,2,3-triazol-1-yl)propan-1-one (Intermediate AT)

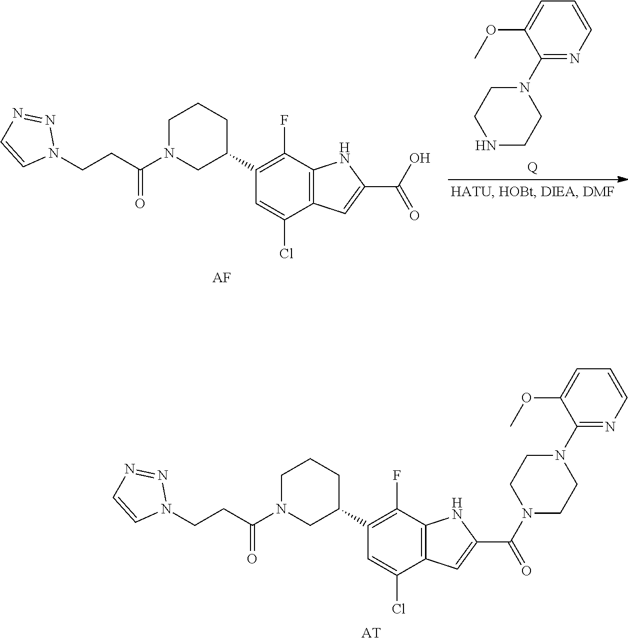

To a solution of (R)-6-(1-(3-(1H-1,2,3-triazol-1-yl)propanoyl)piperidin-3-yl)-4-chloro-7-fluoro-1H-indole-2-carboxylic acid (200 mg, 476 μmol, Intermediate AS) in anhydrous DMF (3 mL) was added HOBt (128 mg, 952 μmol), DIEA (307 mg, 2.38 mmol, 414 μL) and HATU (235 mg, 619 μmol). Then 1-(3-methoxypyridin-2-yl)piperazine (292 mg, 952 μmol, TFA, Intermediate Q) was added and the reaction was stirred at 25° C. for 1 hr under nitrogen atmosphere. On completion, the reaction mixture was diluted with water (10 mL) and extracted with ethyl acetate (2×10 mL). The combined organic layers were washed with brine (20 mL) and dried over anhydrous sodium sulfate, filtered and the filtrate was concentrated in vacuo to give the title compound (400 mg) as a yellow solid. LC-MS (ESI⁺) m/z 595.2 (M+H)⁺.

### 1-(2-Methoxyphenyl)piperazine (CAS #35386-24-4) (Intermediate AU)

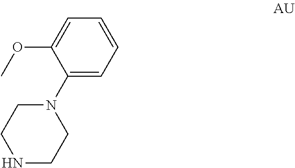

### 1-(2,6-Dimethoxyphenyl)piperazine) (Intermediate AV)

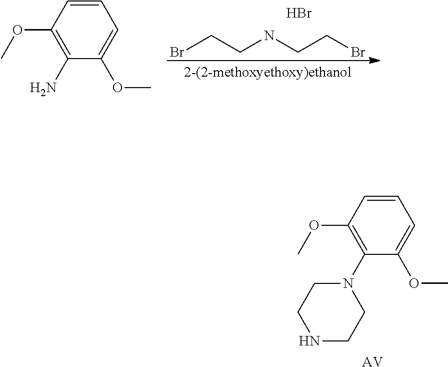

To a solution of 2,6-dimethoxyaniline (1 g, 7 mmol, CAS #2734-70-5), 2-bromo-N-(2-bromoethyl)ethanamine hydrobromide (3.05 g, 9.79 mmol, CAS #43204-63-3) in 2-(2-methoxyethoxy)ethanol (1 mL), then the reaction was stirred at 130° C. for 4 hrs under nitrogen atmosphere. On completion, to the reaction mixture was added acetone, then the mixture was filtered and the filter cake was dried in vacuo to give the title compound (1.2 g) as a white solid. LC-MS (ESI⁺) m/z 223.2 (M+H)⁺.

### 1-(3-Methoxy-6-methylpyridin-2-yl)piperazine (Intermediate AW)

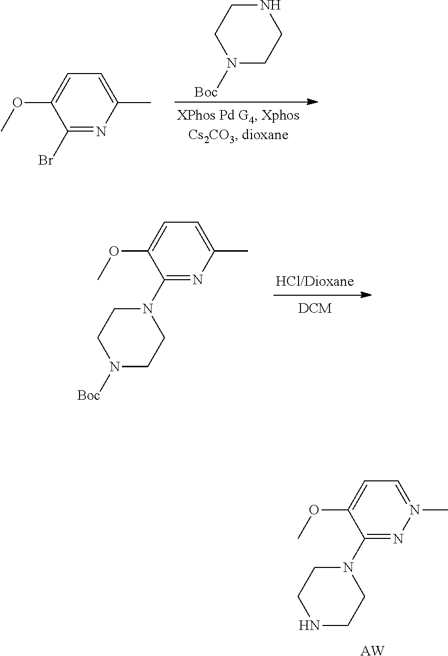

### Step 1—tert-butyl 4-(3-methoxy-6-methylpyridin-2-yl)piperazine-1-carboxylate

To a solution of 2-bromo-3-methoxy-6-methylpyridine (1 g, 5 mmol, CAS #24207-22-5), tert-butyl piperazine-1-carboxylate (1.38 g, 7.42 mmol, CAS #57260-71-6) in dioxane (10 mL) was added Cs₂CO₃ (6.45 g, 19.8 mmol), XPhos (283 mg, 593 μmol) and Xphos Pd G4 (425 mg, 494 μmol). Then the reaction was stirred at 80° C. for 2 hrs under nitrogen atmosphere. On completion, the reaction mixture was filtered and the filtrate was concentrated in vacuo to give the crude residue. The crude residue was purified by column chromatography (SiO₂, Petroleum ether/Ethyl acetate=1/0 to 10/1) to give the title compound (1.2 g, 55% yield) as a yellow solid. LC-MS (ESI⁺) m/z 308.1 (M+H)⁺.

### Step 2—1-(3-Methoxy-6-methylpyridin-2-yl)piperazine

To a solution of tert-butyl tert-butyl 4-(3-methoxy-6-methylpyridin-2-yl)piperazine-1-carboxylate (1 g, 3 mmol) in DCM (10 mL) was added HCl/dioxane (8 M, 3 mL). Then the mixture was stirred at 25° C. for 2 hrs. On completion, the reaction mixture was filtered and the filter cake was concentrated in vacuo to give the title compound (500 mg, HCl) as a white solid. LC-MS (ESI⁺) m/z 208.1 (M+H)⁺.

### 1-(3,5-Dimethoxypyridin-2-yl)piperazine (Intermediate AX)

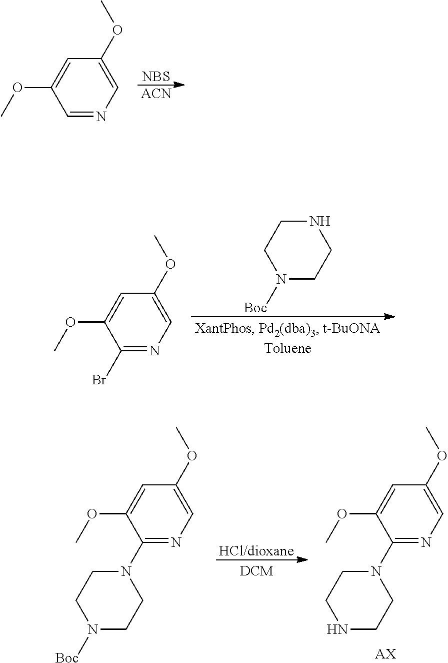

### Step 1—2-Bromo-3,5-dimethoxypyridine

To a solution of 3,5-dimethoxypyridine (5 g, 40 mmol, CAS #18677-48-0) in ACN (50 mL) was added NBS (6.40 g, 35.9 mmol). The mixture was then stirred at 80° C. for 1 hr. On completion, the reaction mixture was diluted with water (50 mL) and extracted with EA (100 mL×2). The combined organic layers were dried over anhydrous sodium sulfate, filtered and concentrated under reduced pressure to give a residue. The residue was purified by column chromatography (SiO₂, Petroleum ether/Ethyl acetate=1/0 to 0/1) to give the title compound (2 g, 25% yield) as a white solid. ¹H NMR (400 MHz, DMSO-d₆) δ=7.73 (d, J=2.8 Hz, 1H), 7.13 (d, J=2.4 Hz, 1H), 3.89 (s, 3H), 3.86 (s, 3H).

### Step 2—Tert-butyl 4-(3,5-dimethoxypyridin-2-yl)piperazine-1-carboxylate

A mixture of 2-bromo-3,5-dimethoxypyridine (500 mg, 2.29 mmol), tert-butyl piperazine-1-carboxylate (427 mg, 2.29 mmol, CAS #57260-71-6), t-BuONa (1.10 g, 11.4 mmol), Xantphos (132 mg, 229 μmol) and Pd₂(dba)₃ (209 mg, 229 μmol) in toluene (10 mL) was degassed and purged with N₂ three times. Then the mixture was stirred at 100° C. for 4 hrs under N₂ atmosphere. On completion, the reaction mixture was concentrated under reduced pressure to give a residue. The residue was purified by column chromatography (SiO₂, Petroleum ether/Ethyl acetate=1/0 to 2/1) to give a title compound (680 mg, 89% yield) as a yellow solid. LC-MS (ESI⁺) m/z 324.3 (M+H)⁺.

### Step 3—1-(3,5-Dimethoxypyridin-2-yl)piperazine

To a solution of tert-butyl 4-(3,5-dimethoxypyridin-2-yl)piperazine-1-carboxylate (680 mg, 2.10 mmol) in DCM (10 mL) was added HCl/dioxane (8 M, 1 mL). The mixture was then stirred at 20° C. for 0.5 hrs. On completion, the reaction mixture was filtered and the filter cake was concentrated under reduced pressure to give the title compound (447 mg, HCl) as a yellow solid. LC-MS (ESI⁺) m/z 224.1 (M+H)⁺.

### 5-Methoxy-N-methyl-6-piperazin-1-yl-pyridin-3-amine (Intermediate AY)

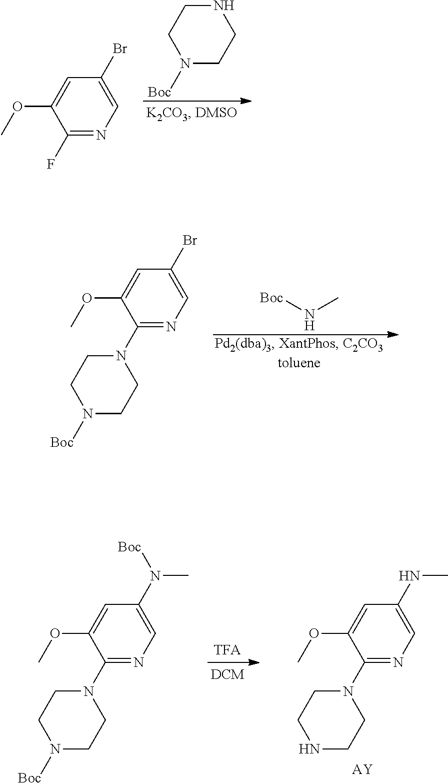

### Step 1—Tert-butyl 4-(5-bromo-3-methoxypyridin-2-yl)piperazine-1-carboxylate

To a solution of 5-bromo-2-fluoro-3-methoxy-pyridine (1.8 g, 8.7 mmol, CAS #880870-66-6) and tert-butyl piperazine-1-carboxylate (1.63 g, 8.74 mmol, CAS #57260-71-6) in DMSO (15 mL) was added K₂CO₃ (3.62 g, 26.2 mmol). The mixture was then stirred at 120° C. for 3 hrs. On completion, the reaction mixture was quenched by addition of H₂O (10 mL), and then extracted with EtOAc 60 mL (20 mL×3). The combined organic layers were washed with brine (20 mL×3), dried over Na₂SO₄, filtered and concentrated under reduced pressure to give a residue. The crude residue was purified by column chromatography (SiO₂, Petroleum ether/Ethyl acetate=20/1 to 5/1) to give the title compound (2.20 g, 66% yield) as a white solid. LC-MS (ESI⁺) m/z 272.0 (M−99)⁺.

### Step 2—Tert-butyl 4-(5-((tert-butoxycarbonyl)(methyl)amino)-3-methoxypyridin-2-yl)piperazine-1-carboxylate

A mixture of tert-butyl 4-(5-bromo-3-methoxy-2-pyridyl)piperazine-1-carboxylate (800 mg, 2.15 mmol), tert-butyl methylcarbamate (422 mg, 3.22 mmol), Cs₂CO₃ (1.75 g, 5.37 mmol), Pd₂(dba)₃ (137 mg, 150 μmol) and Xantphos (186 mg, 322 μmol) in toluene (16 mL) was degassed and purged with N₂ three times. Then the mixture was stirred at 100° C. for 12 h under N₂ atmosphere. On completion, the reaction mixture was concentrated under reduced pressure. The mixture was purified by column chromatography (SiO₂, Petroleum ether/Ethyl acetate=6/1 to 3/1) to give the title compound (370 mg, 41% yield) as a yellow oil. LC-MS (ESI⁺) m/z 423.2 (M+H)⁺. ¹H NMR (400 MHz, DMSO-d₆) δ=1.32-1.44 (m, 18H) 1.99 (s, 1H) 3.16 (s, 3H) 3.17-3.28 (m, 4H) 3.29-3.43 (m, 4H) 3.80 (s, 3H) 7.23 (d, J=2.0 Hz, 1H) 7.73 (d, J=2.0 Hz, 1H).

### Step 3—5-Methoxy-N-methyl-6-piperazin-1-yl-pyridin-3-amine

A solution of tert-butyl 4-[5-[tert-butoxycarbonyl(methyl)amino]-3-methoxy-2-pyridyl]piperazine-1-carboxylate (370 mg, 875 μmol) in TFA (1.54 g, 13.4 mmol, 1 mL) and DCM (2 mL) was stirred at 25° C. for 1 h. On completion, the reaction mixture was filtered and concentrated under reduced pressure to give the title compound (690 mg) as a white solid. LC-MS (ESI⁺) m/z 223.2 (M+H)⁺.

### 1-(5-Cyclopropyl-3-methoxypyridin-2-yl)piperazine (Intermediate AZ)

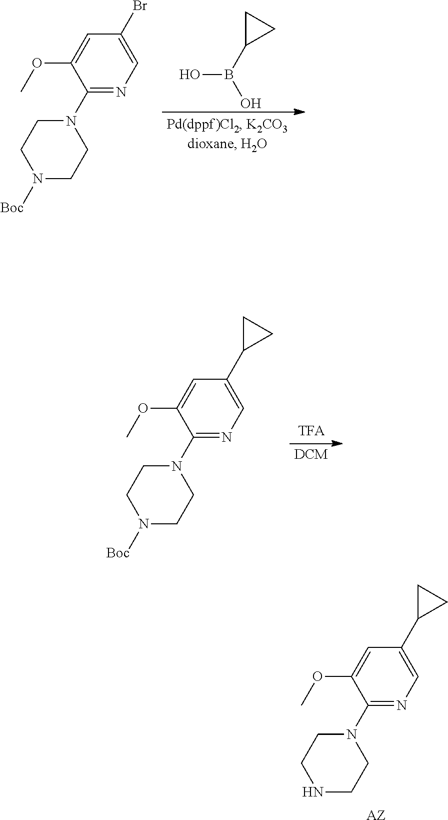

### Step 1—Tert-butyl 4-(5-cyclopropyl-3-methoxypyridin-2-yl)piperazine-1-carboxylate

A mixture of tert-butyl 4-(5-bromo-3-methoxy-2-pyridyl)piperazine-1-carboxylate (600 mg, 1.61 mmol, synthesized via Step 1 of Intermediate AY), cyclopropylboronic acid (277 mg, 3.22 mmol, CAS #411235-57-9), Pd(dppf)Cl₂·CH₂Cl₂ (263.2 mg, 322 μmol), and K₂CO₃ (668.28 mg, 4.84 mmol) in dioxane (5 mL) and H₂O (1 mL) was degassed and purged with N₂ three times. Then the mixture was stirred at 80° C. for 1 hr under N₂ atmosphere. On completion, the reaction mixture was concentrated under reduced pressure to remove solvent. The crude residue was purified by column chromatography (SiO₂, Petroleum ether/Ethyl acetate=2/1 to 1/1) to give the title compound (340 mg, 63% yield) as a white solid. LC-MS (ESI⁺) m/z 334.1 (M+H)⁺.

### Step 2—1-(5-Cyclopropyl-3-methoxypyridin-2-yl)piperazine

To a solution of tert-butyl 4-(5-cyclopropyl-3-methoxy-2-pyridyl)piperazine-1-carboxylate (336 mg, 1.01 mmol) in DCM (4 mL) was added TFA (1.54 g, 13.5 mmol). The mixture was then stirred at 25° C. for 1 hr. On completion, the reaction mixture was concentrated under reduced pressure to remove solvent to give the title compound (300 mg) as a yellow oil. LC-MS (ESI⁺) m/z 234.2 (M+H)⁺.

### 6-(1-(3-(1H-1,2,3-triazol-1-yl)propanoyl)-1,2,5,6-tetrahydropyridin-3-yl)-7-fluoro-4-(3-methoxypyridin-4-yl)-1H-indole-2-carboxylic acid (Intermediate BJ)

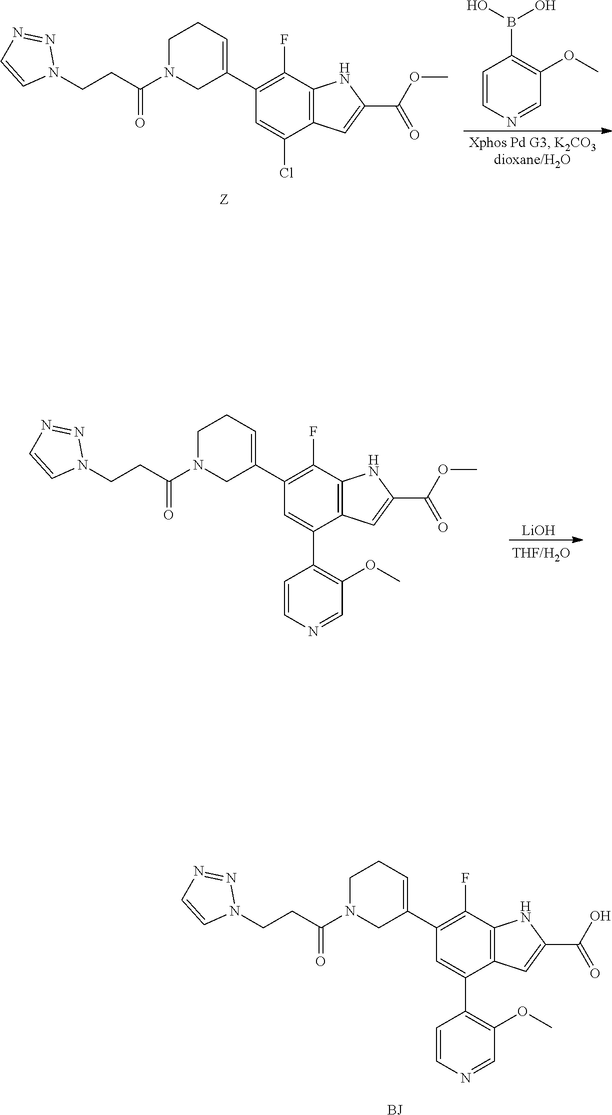

### Step 1—Methyl 6-(1-(3-(1H-1,2,3-triazol-1-yl)propanoyl)-1,2,5,6-tetrahydropyridin-3-yl)-7-fluoro-4-(3-methoxypyridin-4-yl)-1H-indole-2-carboxylate

To a solution of methyl 6-(1-(3-(1H-1,2,3-triazol-1-yl)propanoyl)-1,2,5,6-tetrahydropyridin-3-yl)-4-chloro-7-fluoro-1H-indole-2-carboxylate (750 mg, 1.7 mmol, Intermediate Z) in dioxane (8 mL) and H₂O (2 mL) was added (3-methoxy-4-pyridyl)boronic acid (398 mg, 2.6 mmol, CAS #1008506-24-8), XPhos Pd G3 (147 mg, 173 μmol), and K₃PO₄ (1.1 g, 5.2 mmol). The mixture was degassed and purged with N₂ three times, and then the mixture was stirred at 80° C. for 1 h under N₂ atmosphere. On completion, the residue was purified by prep-HPLC (FA condition) to give the title compound (420 mg, 55% yield, FA) as a yellow solid. LC-MS (ESI⁺) m/z 505.2 (M+H)⁺.

### Step 2—6-(1-(3-(1H-1,2,3-triazol-1-yl)propanoyl)-1,2,5,6-tetrahydropyridin-3-yl)-7-fluoro-4-(3-methoxypyridin-4-yl)-1H-indole-2-carboxylic acid

To a solution of methyl 6-(1-(3-(1H-1,2,3-triazol-1-yl)propanoyl)-1,2,5,6-tetrahydropyridin-3-yl)-7-fluoro-4-(3-methoxypyridin-4-yl)-1H-indole-2-carboxylate (420 mg, 832 μmol) in H₂O (2 mL), THF (2 mL), and MeOH (2 mL) was added LiOH·H₂O (174.67 mg, 4.16 mmol). The mixture was then stirred at 25° C. for 1 hr. On completion, to the reaction mixture was added HCl (1N) until the pH ˜2, then the mixture was filtered. The filtrate was concentrated in vacuo to give the title compound (290 mg, HCl) as a yellow solid. LC-MS (ESI⁺) m/z 491.1 (M+H)⁺.

### 1-(2-Fluoro-6-methoxyphenyl)piperazine (Intermediate BK)

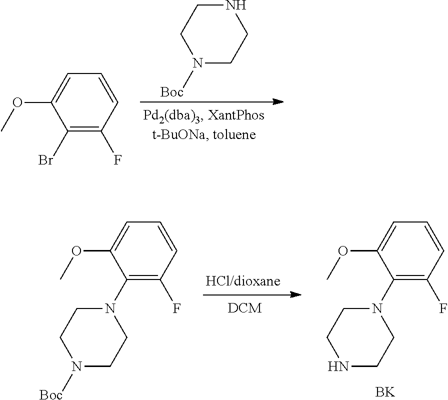

### Step 1—Tert-butyl 4-(2-fluoro-6-methoxyphenyl)piperazine-1-carboxylate

A mixture of 2-bromo-1-fluoro-3-methoxybenzene (908 mg, 4.88 mmol, CAS #446-59-3), tert-butyl piperazine-1-carboxylate (1 g, 5 mmol, CAS #57260-71-6), t-BuONa (2.34 g, 24.4 mmol), Xantphos (282 mg, 488 μmol) and Pd₂(dba)₃ (447 mg, 488 μmol) in toluene (10 mL) was degassed and purged with N₂ three times. Then the mixture was stirred at 100° C. for 2 hrs under N₂ atmosphere. On completion, the reaction mixture was concentrated under reduced pressure to give a residue. The residue was purified by column chromatography (SiO₂, Petroleum ether:Ethyl acetate=10/1 to 5/1) to give the title compound (1 g, 46% yield) as a yellow solid. LC-MS (ESI⁺) m/z 311.1 (M+H)⁺.

### Step 2—1-(2-Fluoro-6-methoxyphenyl)piperazine

To a solution of tert-butyl 4-(2-fluoro-6-methoxyphenyl)piperazine-1-carboxylate (0.7 g, 2.3 mmol) in DCM (2 mL) was added HCl/dioxane (8 M, 2 mL). The mixture was then stirred at 25° C. for 1 hr. On completion, the reaction mixture was concentrated under reduced pressure to give a residue. The crude residue was purified by reversed-phase HPLC (0.8 g/L ammonium bicarbonate) to give the title compound (0.2 g, 41% yield) as a brown solid. LC-MS (ESI⁺) m/z 211.1 (M+H)⁺.

### 1-(3-Methoxypyridin-2-yl)piperazine (Intermediate BL)

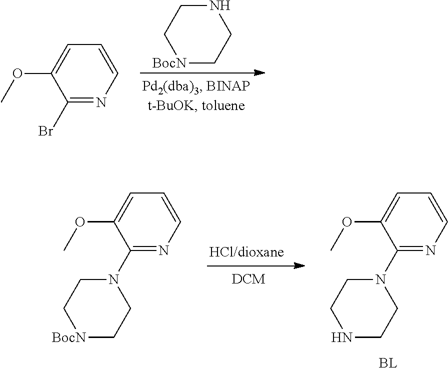

### Step 1—Tert-butyl 4-(3-methoxypyridin-2-yl)piperazine-1-carboxylate

A mixture of tert-butyl piperazine-1-carboxylate (496 mg, 2.66 mmol, CAS #57260-71-6), 2-bromo-3-methoxy-pyridine (0.5 g, 2.66 mmol, CAS #24100-18-3), Pd₂(dba)₃ (244 mg, 266 μmol), BINAP (331 mg, 532 μmol) and t-BuOK (597 mg, 5.32 mmol) in toluene (10 mL) was degassed and purged with N₂ three times. Then the mixture was stirred at 110° C. for 12 hrs under N₂ atmosphere. On completion, the mixture was concentrated under reduce pressure to give a residue. The mixture was purified by reversed-phase HPLC(0.1% FA) to give the title compound (0.5 g, 62% yield) as a white solid. LC-MS (ESI⁺) m/z 294.0 (M+H)⁺.

### Step 2—1-(3-Methoxypyridin-2-yl)piperazine

To a solution of tert-butyl 4-(3-methoxy-2-pyridyl)piperazine-1-carboxylate (0.2 g, 682 μmol) in DCM (2 mL) was added HCl/dioxane (4 M, 1 mL). The mixture was then stirred at 25° C. for 1 hr. On completion, the mixture was concentrated under reduce pressure to give the title compound (120 mg) as a yellow solid. LC-MS (ESI⁺) m/z 294.0 (M+H)⁺.

### 6-(1-(3-(1H-1,2,3-triazol-1-yl)propanoyl)-1,2,5,6-tetrahydropyridin-3-yl)-7-fluoro-4-(4-methoxypyridin-3-yl)-1H-indole-2-carboxylic acid (Intermediate BM)

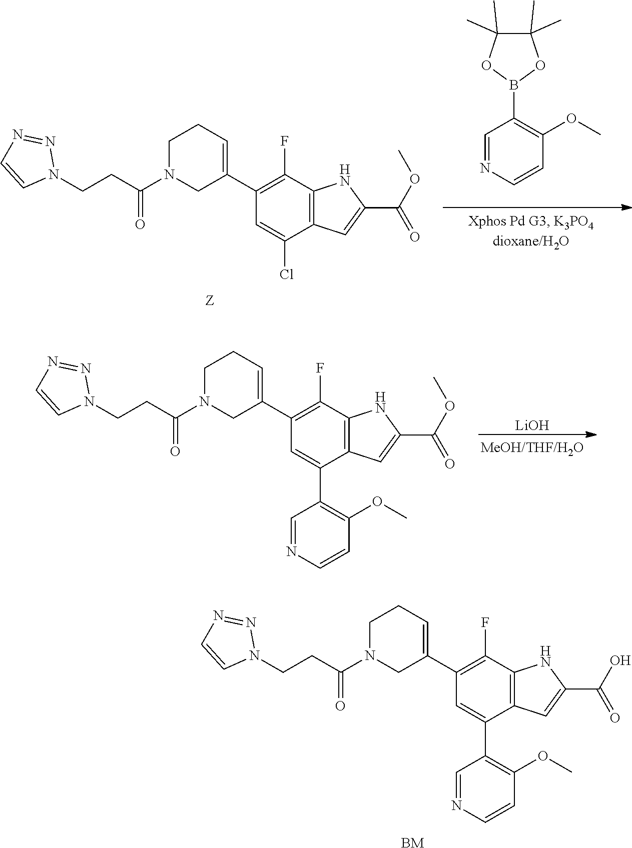

### Step 1—Methyl 6-(1-(3-(1H-1,2,3-triazol-1-yl)propanoyl)-1,2,5,6-tetrahydropyridin-3-yl)-7-fluoro-4-(4-methoxypyridin-3-yl)-1H-indole-2-carboxylate

A mixture of 4-methoxy-3-(4,4,5,5-tetramethyl-1,3,2-dioxaborolan-2-yl)pyridine (272 mg, 1.16 mmol, CAS #758699-74-0), methyl 4-chloro-7-fluoro-6-[1-[3-(triazol-1-yl)propanoyl]-3,6-dihydro-2H-pyridin-5-yl]-1H-indole-2-carboxylate (0.5 g, 1.16 mmol, Intermediate Z), XPhos Pd G3 (98.0 mg, 116 μmol), and K₃PO₄ (614 mg, 2.89 mmol) in H₂O (1 mL) and dioxane (5 mL) was degassed and purged with N₂ three times. Then the mixture was stirred at 80° C. for 2 hrs under N₂ atmosphere. On completion, the mixture was concentrated under reduce pressure to give a residue The mixture was purified by reversed-phase HPLC (0.1% FA) to give the title compound (230 mg, 37% yield) as a white solid. LC-MS (ESI⁺) m/z 505.2 (M+H)⁺.

### Step 2—6-(1-(3-(1H-1,2,3-triazol-1-yl)propanoyl)-1,2,5,6-tetrahydropyridin-3-yl)-7-fluoro-4-(4-methoxypyridin-3-yl)-1H-indole-2-carboxylic acid

To a solution of methyl 7-fluoro-4-(4-methoxy-3-pyridyl)-6-[1-[3-(triazol-1-yl)propanoyl]-3,6-dihydro-2H-pyridin-5-yl]-1H-indole-2-carboxylate (0.23 g, 456 μmol) in THF (1 mL), MeOH (1 mL) and H₂O (1 mL) was added LiOH·H₂O (95.7 mg, 2.28 mmol). The mixture was then stirred at 25° C. for 1 hr. On completion, the mixture was quenched by 2M HCl (5 mL) at 25° C. then extracted by EA (50×3 mL). The mixture was concentrated under reduce pressure to give a residue to give the title compound (180 mg) as a yellow solid. LC-MS (ESI⁺) m/z 491.2 (M+H)⁺.

### 1-(5-Chloro-3-methoxypyridin-2-yl)piperazine (Intermediate BN)

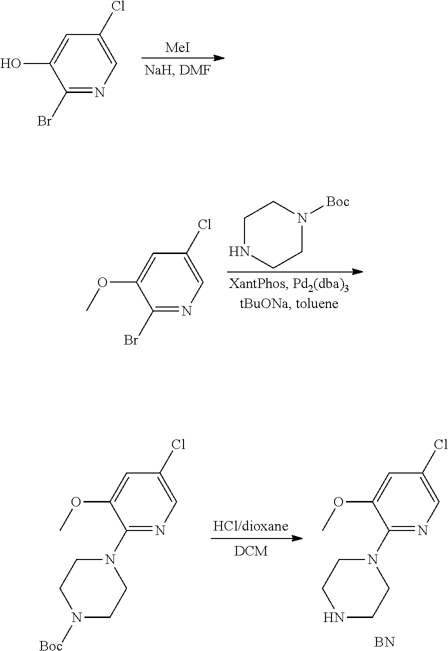

### Step 1—2-Bromo-5-chloro-3-methoxypyridine

To a mixture of 2-bromo-5-chloropyridin-3-ol (1 g, 4.80 mmol, CAS #286946-77-8) in DMF (10 mL) was added NaH (249 mg, 6.24 mmol, 60% dispersion in mineral oil) and then the mixture was stirred at 0° C. for 0.5 hours under N₂ atmosphere. Next, MeI (612.86 mg, 4.32 mmol, 268.80 μL) was added and the mixture was stirred at 25° C. for 11.5 hrs under N₂ atmosphere. On completion, the crude product was concentrated under reduced pressure to give a residue. The residue was purified by column chromatography (SiO₂, Petroleum ether/Ethyl acetate=1/0 to 4/1) to give the title compound (600 mg, 65% yield) as a white solid.

### Step 2—Tert-butyl 4-(5-chloro-3-methoxypyridin-2-yl)piperazine-1-carboxylate

A mixture of 2-bromo-5-chloro-3-methoxypyridine (450 mg, 2.02 mmol), tert-butyl piperazine-1-carboxylate (452 mg, 2.43 mmol, CAS #57260-71-6), Xantphos (234 mg, 405 μmol), Pd₂(dba)₃ (185 mg, 202 μmol) and tBuONa (389 mg, 4.05 mmol) in toluene (5 mL) was degassed and purged with N₂ three times. Then the mixture was stirred at 90° C. for 2 hrs under N₂ atmosphere. On completion, the crude product was concentrated under reduced pressure to give a residue. The residue was purified by prep-HPLC to give the title compound (400 mg, 50% yield) as a white solid. LC-MS (ESI⁺) m/z 328.0 (M+H)⁺.

### Step 3—1-(5-Chloro-3-methoxypyridin-2-yl)piperazine

To a solution of tert-butyl 4-(5-chloro-3-methoxypyridin-2-yl)piperazine-1-carboxylate (330 mg, 1.01 mmol) in DCM (3 mL) was added HCl/dioxane (8 M, 125.84 μL). The mixture was then stirred at 25° C. for 2 hrs. On completion, the mixture was concentrated under reduced pressure to give the title compound (200 mg, HCl salt) as a white solid. LC-MS (ESI⁺) m/z 228.1 (M+H)⁺.

### 1-(5-Fluoro-3-methoxypyridin-2-yl)piperazine (Intermediate BO)

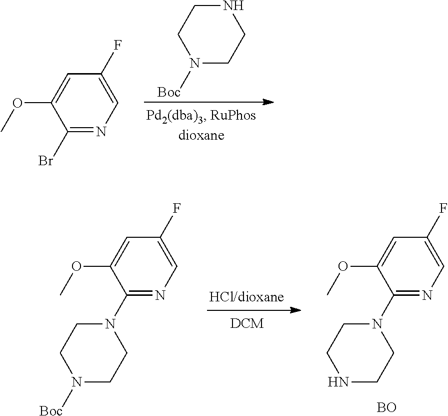

### Step 1—Tert-butyl 4-(5-fluoro-3-methoxypyridin-2-yl)piperazine-1-carboxylate

A mixture of 2-bromo-5-fluoro-3-methoxy-pyridine (1 g, 5 mmol, CAS #1256806-73-1), tert-butyl piperazine-1-carboxylate (994 mg, 5.34 mmol, CAS #57260-71-6), Cs₂CO₃ (4.74 g, 14.6 mmol), RuPhos (453 mg, 971 μmol) and Pd₂(dba)₃ (445 mg, 485 μmol) in dioxane (20 mL) was degassed and purged with N₂ three times. Then the mixture was stirred at 100° C. for 12 hrs under N₂ atmosphere. On completion, the reaction mixture was concentrated under reduced pressure to give a residue. The residue was purified by column chromatography (SiO₂, Petroleum ether/Ethyl acetate=1/0 to 0/1) to give the title compound (1.2 g, 74% yield) as a yellow oil. LC-MS (ESI⁺) m/z 312.1 (M+H)⁺.

### Step 2—1-(5-Fluoro-3-methoxypyridin-2-yl)piperazine

To a solution of tert-butyl 4-(5-fluoro-3-methoxy-2-pyridyl)piperazine-1-carboxylate (470 mg, 1.51 mmol) in DCM (8 mL) was added HCl/dioxane (8 M, 2 mL). The mixture was then stirred at 25° C. for 2 hrs. On completion, the reaction mixture was concentrated under reduced pressure to give the title compound (452 mg, HCl) as a yellow solid. LC-MS (ESI⁺) m/z 212.1 (M+H)⁺.

### (2,3-Difluorophenyl)boronic acid (CAS #121219-16-7) (Intermediate BP)

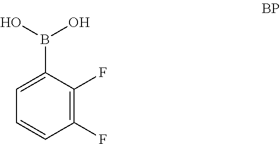 [SMILES: OB(O)c1cccc(F)c1F]

### 6-(1-(3-(1H-1,2,3-triazol-1-yl)propanoyl)-1,2,5,6-tetrahydropyridin-3-yl)-4-chloro-7-fluoro-1H-indole-2-carboxylic acid (Intermediate BQ)

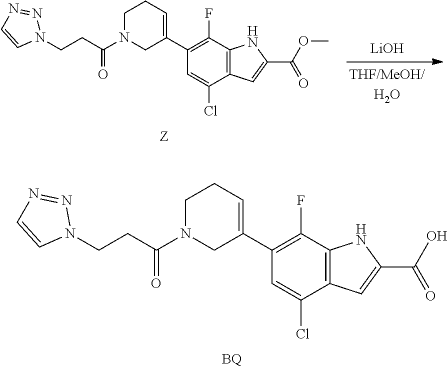

To a solution of methyl 6-(1-(3-(1H-1,2,3-triazol-1-yl)propanoyl)-1,2,5,6-tetrahydropyridin-3-yl)-4-chloro-7-fluoro-1H-indole-2-carboxylate (4 g, 9 mmol, Intermediate Z) in THF (20 mL), MeOH (5 mL) and H₂O (5 mL) was added LiOH·H₂O (1.17 g, 27.8 mmol). The mixture was then stirred at 25° C. for 4 h. On completion, the reaction mixture was quenched by addition of 6N HCl to adjust pH to 4-5, and then the mixture was extracted with EA (20 mL×3). The combined organic layers were washed with brine (30 mL), dried over Na₂SO₄, filtered and concentrated under reduced pressure to give the title compound (3 g, 54% yield) as a yellow solid. LC-MS (ESI⁺) m/z 418.0 (M+H)⁺.

### 1-(5-(4-Chloro-7-fluoro-2-(4-(3-methoxypyridin-2-yl)piperazine-1-carbonyl)-1H-indol-6-yl)-3,6-dihydropyridin-1(2H)-yl)-3-(1H-1,2,3-triazol-1-yl)propan-1-one (Intermediate BR)

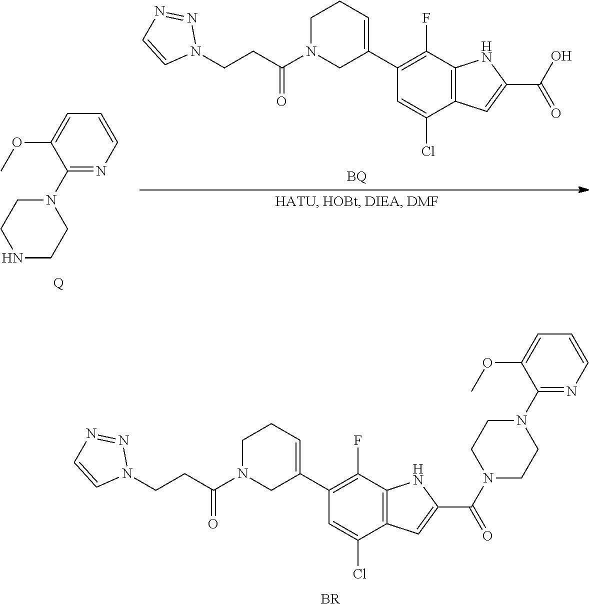

To a solution of 4-chloro-7-fluoro-6-[1-[3-(triazol-1-yl)propanoyl]-3,6-dihydro-2H-pyridin-5-yl]-1H-indole-2-carboxylic acid (500 mg, 838 μmol, Intermediate BQ) in DMF (4 mL) was added HOBt (226 mg, 1.68 mmol), HATU (350 mg, 921 μmol) and DIEA (350 mg, 921 μmol). Then 1-(3-methoxy-2-pyridyl)piperazine (770 mg, 3.35 mmol, HCl, Intermediate Q) was added and the mixture was stirred at 25° C. for 1 hr. On completion, the reaction mixture was concentrated under reduced pressure to remove solvent. The crude residue was purified by reversed-phase HPLC (0.8% g/L NH₄HCO₃ condition) to give the title compound (320 mg, 61% yield) as a white solid. LC-MS (ESI⁺) m/z 593.2 (M+H)⁺.

### 7-Bromo-5-iodo-N,N-dimethylbenzofuran-2-carboxamide (Intermediate BS)

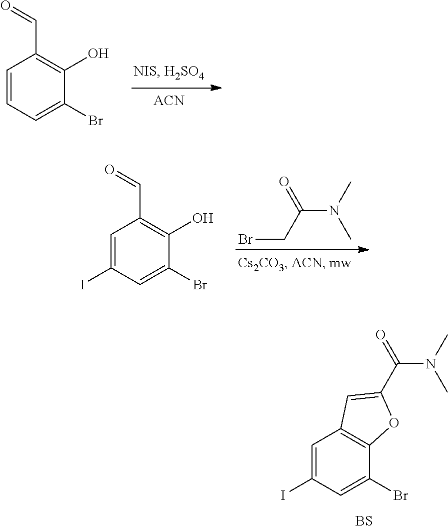

### Step 1—3-Bromo-2-hydroxy-5-iodobenzaldehyde

To a solution of 3-bromo-2-hydroxy-benzaldehyde (5 g, 25 mmol, CAS #1829-34-1) in ACN (100 mL) was added H₂SO₄ (2.56 g, 26.1 mmol, 1.39 mL), then the mixture was stirred at 25° C. for 30 min. Next, NIS (5.88 g, 26.1 mmol) was added, and the mixture was stirred at 25° C. for 10 h. On completion, the reaction mixture was poured into ice water (100 mL) and extracted with ethyl acetate (100 mL×3). The combined organic layers were washed with brine (100 mL×3), dried over sodium sulfate, filtered and concentrated under reduced pressure to give a residue. The residue was purified by column chromatography (SiO₂, Petroleum ether/Ethyl acetate=1/0) to afford the title compound (8 g, 98% yield) as a yellow solid. ¹H NMR (400 MHz, CDCl₃) δ=11.53 (s, 1H), 9.81 (s, 1H), 8.06 (d, J=2.0 Hz, 1H), 7.83 (d, J=2.0 Hz, 1H).

### Step 2—7-Bromo-5-iodo-N,N-dimethylbenzofuran-2-carboxamide

To a solution of 3-bromo-2-hydroxy-5-iodo-benzaldehyde (1.00 g, 3.06 mmol), 2-bromo-N,N-dimethyl-acetamide (609 mg, 3.67 mmol, CAS #5468-77-9) in ACN (10 mL) was added Cs₂CO₃ (2.49 g, 7.65 mmol). The mixture was then stirred at 120° C. for 30 min under microwave. On completion, the reaction mixture was poured into ice water (100 mL) and extracted with ethyl acetate (100 mL×3). The combined organic layers were washed with brine (100 mL×3), dried over sodium sulfate, filtered and concentrated under reduced pressure to give a residue, The residue was purified by flash silica gel chromatography (ISCO®; 40 g SepaFlash® Silica Flash Column, Eluent of 0˜20% Ethyl acetate/Petroleum ether gradient @80 mL/min) to afford the title compound (2.9 g, 34% yield) as a yellow solid. ¹H NMR (400 MHz, CDCl₃) δ 7.94 (d, J=1.6 Hz, 1H), 7.85 (d, J=1.6 Hz, 1H), 7.34 (s, 1H), 3.43-3.35 (m, 3H), 3.16 (br s, 3H).

### 5-((Tert-butoxycarbonyl)amino)-2-formyl-1,2,3,4-tetrahydroisoquinoline-3-carboxylic acid (Intermediate BT)

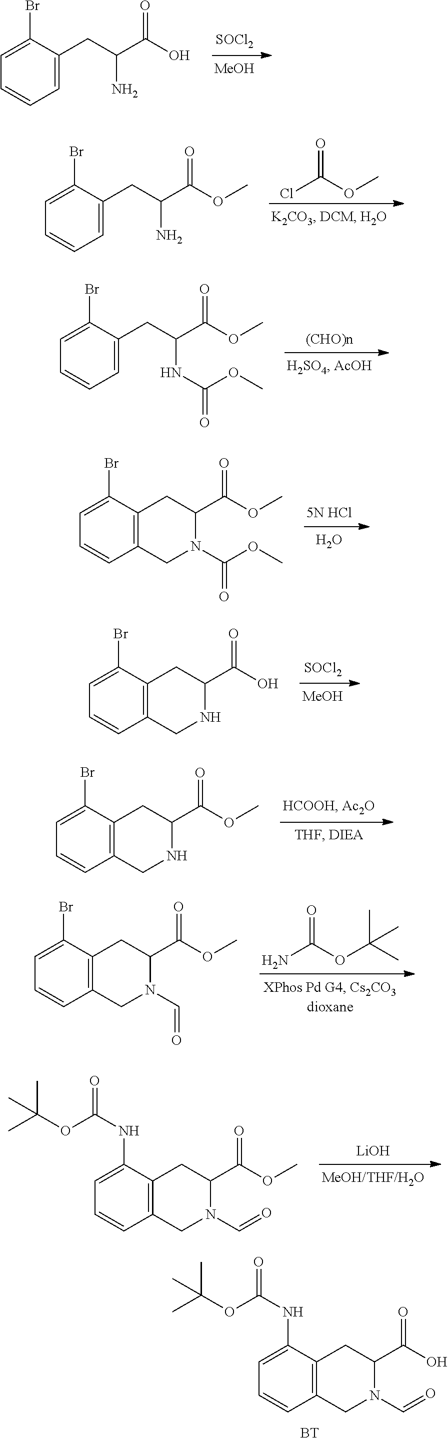

### Step 1—Methyl 2-amino-3-(2-bromophenyl)propanoate

To a solution of 2-amino-3-(2-bromophenyl)propanoic acid (20.0 g, 81.9 mmol, CAS #1191-79-3) in MeOH (200 mL) was dropwise added SOCl₂ (29.2 g, 246 mmol) at 0° C., then the mixture was stirred at 60° C. for 12 hrs under N₂ atmosphere. On completion, the reaction mixture was concentrated under reduced pressure to give the title compound (23.0 g, 94% yield) as a white solid. LC-MS (ESI⁺) m/z 257.8 (M+H)⁺; ¹H NMR (400 MHz, DMSO-d₆) δ=8.95 (s, 3H), 7.62 (d, J=8.0 Hz, 1H), 7.48-7.33 (m, 2H), 7.23 (t, J=7.2 Hz, 1H), 4.11 (d, J=6.4 Hz, 1H), 3.59 (s, 3H), 3.37 (J=6.0, 13.7 Hz, 1H), 3.29-3.13 (m, 1H).

### Step 2—Methyl 3-(2-bromophenyl)-2-((methoxycarbonyl)amino)propanoate

To a solution of methyl 2-amino-3-(2-bromophenyl)propanoate (23.0 g, 78.1 mmol) and Na₂CO₃ (33.1 g, 312 mmol) in H₂O (200 mL) was added DCM (200 mL) in 2 L three-necked flask and the mixture was stirred at 0° C. for 30 min. Then, methyl carbonochloridate (14.6 g, 155 mmol, CAS #79-22-1) was added at 0° C., and the mixture was allowed to warm up to 25° C. for 1.5 hrs while stirring. On completion, the reaction mixture was diluted with ice water (600 mL), and extracted with DCM (200 mL×2). The combined organic layers were dried over anhydrous Na₂SO₄, filtered and concentrated under reduced pressure to give the title compound (25.0 g) as a gray solid. LC-MS (ESI⁺) m/z 315.0 (M+H)⁺. ¹H NMR (400 MHz, DMSO-d₆) δ=7.76 (d, J=8.4 Hz, 1H), 7.59 (d, J=8.0 Hz, 1H), 7.38-7.27 (m, 2H), 7.24-7.12 (m, 1H), 4.41-4.28 (m, 1H), 3.63 (s, 3H), 3.47 (s, 3H), 3.20 (dd, J=5.2, 13.8 Hz, 1H), 2.95 (dd, J=10.4, 13.8 Hz, 1H).

### Step 3—Methyl 3-(2-bromophenyl)-2-((methoxycarbonyl)amino)propanoate

To a solution methyl 3-(2-bromophenyl)-2-(methoxycarbonylamino)propanoate (25.0 g, 79.1 mmol) in AcOH (188 mL) was added (CHO)n (4.75 g, 158 mmol) and H₂SO₄ (115 g, 1.17 mol, 62.5 mL), then the mixture was stirred at 25° C. for 1.5 hrs. On completion, the reaction mixture was diluted with water (600 mL) and extracted with EA (200 mL×3). The combined organic layers were washed with brine (300 mL×2), dried over Na₂SO₄, filtered and concentrated under reduced pressure to give a residue. The residue was purified by flash silica gel chromatography (ISCO®; 120 g SepaFlash® Silica Flash Column, Eluent of 5˜20% Ethyl acetate/Petroleum ether gradient @80.0 mL/min) to give the title compound (23.0 g, 84% yield) as a colorless oil. LC-MS (ESI⁺) m/z 329.8 (M+H)⁺; ¹H NMR (400 MHz, DMSO-d₆) δ=8.35-8.16 (m, 1H), 7.57 (dd, J=7.2, 13.2 Hz, 1H), 7.38-7.29 (m, 1H), 7.22 (td, J=7.2, 12.4 Hz, 1H), 5.11-4.76 (m, 1H), 4.75-4.27 (m, 2H), 3.97-3.62 (m, 1H), 3.55 (d, J=11.2 Hz, 3H), 3.27-3.05 (m, 1H), 1.44-1.18 (m, 12H).

### Step 4—5-Bromo-1,2,3,4-tetrahydroisoquinoline-3-carboxylic acid

To a solution of dimethyl 5-bromo-3,4-dihydro-1H-isoquinoline-2,3-dicarboxylate (22.0 g, 67.0 mmol) in H₂O (90.0 mL) was added HCl (12.0 M, 160 mL), then the mixture was stirred at 105° C. for 24 hours. On completion, the mixture was cooled to 0° C., then filtered and the filter cake was dried to give the title compound (15.7 g, 80% yield) as a white solid. LC-MS (ESI⁺) m/z 255.9 (M+H)⁺.

### Step 5—Methyl 5-bromo-1,2,3,4-tetrahydroisoquinoline-3-carboxylate

To a solution of 5-bromo-1,2,3,4-tetrahydroisoquinoline-3-carboxylic acid (15.0 g, 58.6 mmol) in MeOH (150 mL) was added SOCl₂ (13.9 g, 117 mmol) at 0° C., then the mixture was stirred at 60° C. for 12 hrs. On completion, the reaction mixture was concentrated under reduced pressure to give the title compound (17.5 g, 97% yield) as a white solid. LC-MS (ESI⁺) m/z 271.8 (M+H)⁺; ¹H NMR (400 MHz, DMSO-d₆) δ=10.72-10.19 (m, 2H), 7.61 (d, J=7.2 Hz, 1H), 7.36-7.30 (m, 1H), 7.28-7.22 (m, 1H), 4.71-4.53 (m, 1H), 4.44-4.27 (m, 2H), 3.83 (s, 3H), 3.27 (dd, J=5.2, 17.3 Hz, 1H), 3.04 (dd, J=11.2, 17.2 Hz, 1H).

### Step 6—Methyl 5-bromo-2-formyl-1,2,3,4-tetrahydroisoquinoline-3-carboxylate

To a solution of methyl 5-bromo-1,2,3,4-tetrahydroisoquinoline-3-carboxylate (17.5 g, 57.1 mmol) in THF (120 mL) was added DIEA (29.5 g, 228 mmol). Then a mixture of Ac₂O (29.1 g, 285 mmol) and HCOOH (30.3 mL) was added and the mixture was stirred at 20° C. for 3 hrs. On completion, the reaction mixture was diluted with water (800 mL) and extracted with EA (50.0 mL×2). The combined organic layers were successively washed with saturated sodium bicarbonate solution (200 mL), HCl (0.5 M, 50.0 mL) and brine (100 ml×2), dried over Na₂SO₄, filtered and concentrated under reduced pressure to give the title compound (16.0 g, 91% yield) as a light-yellow solid. LC-MS (ESI⁺) m/z 299.8 (M+H)⁺; ¹H NMR (400 MHz, DMSO-d₆) δ=8.38-8.19 (m, 1H), 7.59-7.45 (m, 1H), 7.31-7.09 (m, 2H), 5.23-5.13 (m, 1H), 4.98-4.72 (m, 1H), 4.61-4.15 (m, 1H), 3.68-3.56 (m, 3H), 3.48-3.33 (m, 1H), 3.12-2.99 (m, 1H)

### Step 7—Ethyl 5-((tert-butoxycarbonyl)amino)-2-formyl-1,2,3,4-tetrahydroisoquinoline-3-carboxylate

A mixture of methyl 5-bromo-2-formyl-1,2,3,4-tetrahydroisoquinoline-3-carboxylate (500 mg, 1.68 mmol), tert-butyl carbamate (589 mg, 5.03 mmol), Cs₂CO₃ (1.64 g, 5.03 mmol), and Xphos Pd G4 (144 mg, 167 umol) in dioxane (8 mL) was degassed and purged with N₂ three times. Then the mixture was stirred at 90° C. for 2 h under N₂ atmosphere. On completion, the mixture was diluted with water (50 mL), extracted with ethyl acetate (50 mL×2), dried over sodium sulfate, filtered and concentrated under reduced pressure. The residue was purified by silica gel column chromatography (petroleum ether/ethyl acetate=10/1˜ethyl acetate) to give the title compound (420 mg, 71% yield) as a white solid. LC-MS (ESI⁺) m/z 357.0 (M+Na)⁺; ¹H NMR (400 MHz, DMSO-d6) δ=8.77 (br d, J=16.0 Hz, 1H), 8.37-8.14 (m, 1H), 7.23-7.12 (m, 2H), 7.07-6.95 (m, 1H), 5.22-4.65 (m, 2H), 4.56-4.15 (m, 1H), 3.66-3.50 (m, 3H), 3.24-2.91 (m, 2H), 1.46 (d, J=1.2 Hz, 9H).

### Step 8—5-((Tert-butoxycarbonyl)amino)-2-formyl-1,2,3,4-tetrahydroisoquinoline-3-carboxylic acid

To a solution of methyl 5-(tert-butoxycarbonylamino)-2-formyl-3,4-dihydro-1H-isoquinoline-3-carboxylate (420 mg, 1.26 mmol) in THF (4 mL) was added LiOH·H₂O (105 mg, 2.51 mmol), H₂O (1 mL) and MeOH (1.5 mL). The mixture was then stirred at 25° C. for 3 h. On completion, the reaction mixture was cooled to 0° C., the reaction mixture was adjusted with 1M aqueous hydrochloric acid solution to pH=2, and extracted with ethyl acetate (20 mL×2). The combined organic layer was dried over sodium sulfate, filtered and the filtrate was concentrated under reduced pressure to give the title compound (250 mg) as a white solid.

### N,N-dimethyl-5-(1,2,5,6-tetrahydropyridin-3-yl)benzofuran-2-carboxamide (Intermediate BU)

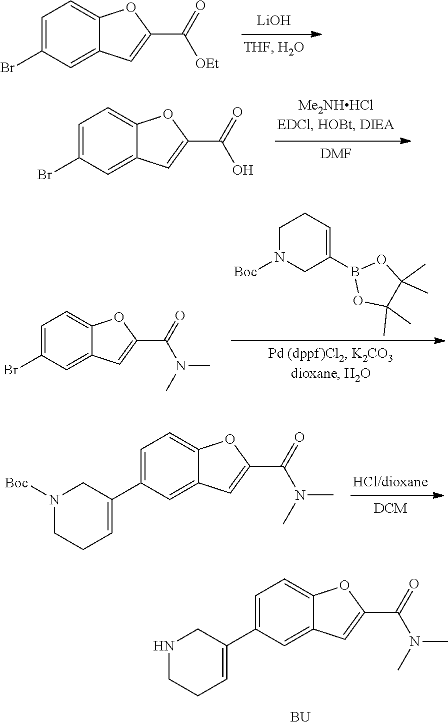

### Step 1—5-Bromobenzofuran-2-carboxylic acid

To a solution of ethyl 5-bromobenzofuran-2-carboxylate (25 g, 93 mmol, CAS #84102-69-2) in THF (200 mL) was added LiOH·H₂O (11.7 g, 279 mmol) in H₂O (50.0 mL), then the mixture was stirred at 25° C. for 3 hrs. On completion, the reaction mixture was adjusted to pH=5-6 with aq. HCl (1 M), filtered and the filter cake was dried to give the title compound (22 g, 97% yield) as a white solid.

### Step 2—5-Bromo-N,N-dimethylbenzofuran-2-carboxamide

To a solution of 5-bromobenzofuran-2-carboxylic acid (22.0 g, 91.3 mmol), EDCI (22.8 g, 119 mmol) and HOBt (16.0 g, 119 mmol) in DMF (220 mL) was added DIEA (59.0 g, 456 mmol) and the mixture was stirred for 30 min at 20° C. Then N-methylmethanamine hydrochloride (11.2 g, 137 mmol) was added and the mixture was stirred at 20° C. for 12 hos. On completion, the reaction mixture was diluted with water (2000 mL) and extracted with EA (300 mL×3). The combined organic layers were washed with brine (300 mL×2), dried over Na₂SO₄, filtered and concentrated under reduced pressure to give a residue. The residue was purified by flash silica gel chromatography (ISCO®; 120 g SepaFlash® Silica Flash Column, Eluent of 10˜30% Ethyl acetate/Petroleum ether gradient @80 mL/min) to give the title compound (6 g, 24% yield) as a yellow solid. LC-MS (ESI⁺) m/z 269.8 (M+H)⁺.

### Step 3—Tert-butyl 5-(2-(dimethylcarbamoyl)benzofuran-5-yl)-3,6-dihydropyridine-1(2H)-carboxylate

To a solution of 5-bromo-N,N-dimethylbenzofuran-2-carboxamide (6.00 g, 22.4 mmol) and tert-butyl 5-(4,4,5,5-tetramethyl-1,3,2-dioxaborolan-2-yl)-3,6-dihydropyridine-1(2H)-carboxylate (7.61 g, 24.6 mmol, CAS #885693-20-9) in dioxane (60.0 mL) was added K₃PO₄ (14.3 g, 67.1 mmol) in H₂O (10.0 mL), then Pd(dppf)Cl₂ (1.64 g, 2.24 mmol) was added under N₂ atmosphere. The mixture was then stirred at 80° C. for 12 hrs under N₂ atmosphere. On completion, the reaction mixture was diluted with water (600 mL) and extracted with ethyl acetate (100 mL×3). The combined organic layers were washed with brine (200 mL×2), dried over Na₂SO₄, filtered and concentrated under reduced pressure to give a residue. The residue was purified by flash silica gel chromatography (ISCO®; 40 g SepaFlash® Silica Flash Column, Eluent of 10˜50% Ethyl acetate/Petroleum ether gradient @30 mL/min) to give the title compound (5.8 g, 70% yield) as a yellow solid. LC-MS (ESI⁺) m/z 371.1 (M+H)⁺.

### Step 4—N,N-dimethyl-5-(1,2,5,6-tetrahydropyridin-3-yl)benzofuran-2-carboxamide

To a solution of tert-butyl 5-(2-(dimethylcarbamoyl)benzofuran-5-yl)-3,6-dihydropyridine-1(2H)-carboxylate (5.80 g, 15.7 mmol) in DCM (40.0 mL) was added HCl/dioxane (4 M, 19.3 mL), then the mixture was stirred at 20° C. for 12 hrs. On completion, the reaction mixture was concentrated under reduced pressure to give the title compound (4.5 g, 92% yield) as a yellow solid.

### 5-Bromo-4-fluorobenzofuran-2-carboxylic acid (Intermediate BV)

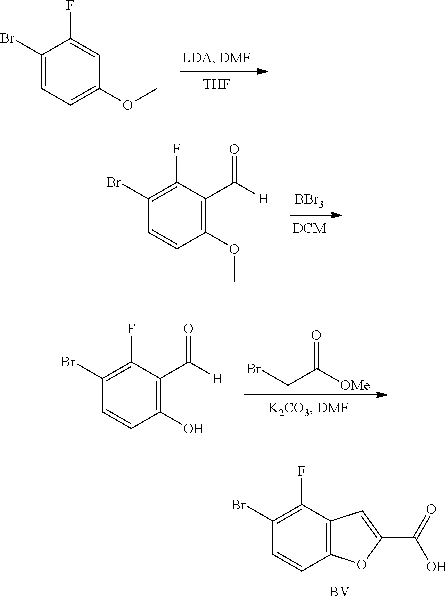

### Step 1—3-Bromo-2-fluoro-6-methoxybenzaldehyde

To a solution of 1-bromo-2-fluoro-4-methoxybenzene (20 g, 100 mmol, CAS #408-50-4) in THF (200 mL) was added dropwise LDA (2 M, 60.0 mL) at −70° C. After addition, the mixture was stirred at this temperature for 30 min, and then DMF (9.50 g, 130 mmol) was added dropwise at −70° C. The resulting mixture was stirred at −70° C. for 30 min. On completion, the reaction mixture was quenched by addition of saturated NH₄Cl aqueous solution (500 ml) and extracted with ethyl acetate (3×300 mL). The combined organic layers were washed with brine (2×300 mL) and dried over anhydrous sodium sulfate, filtered and the filtrate was concentrated in vacuo to give the title compound (20 g) as a yellow solid. LC-MS (ESI⁺) m/z 232.9 (M+H)⁺; ¹H NMR (400 MHz, DMSO-d₆) δ=10.23 (d, J=1.2 Hz, 1H), 7.91 (dd, J=8.4, 8.8 Hz, 1H), 7.14-6.98 (m, 1H), 3.92 (s, 3H).

### Step 2—3-Bromo-2-fluoro-6-hydroxybenzaldehyde

To a solution of 3-bromo-2-fluoro-6-methoxybenzaldehyde (20 g, 90 mmol) in DCM (700 mL) was degassed and purged with N₂ three times, and then BBr₃ (64.5 g, 257 mmol) was added to the mixture at 0° C. The mixture was then stirred at 25° C. for 4 hrs under N₂ atmosphere. On completion, the reaction mixture was quenched with ice water (1 L) and extracted with dichloromethane (3×500 mL). The combined organic layers were washed with brine (2×500 mL) and dried over anhydrous sodium sulfate, filtered and the filtrate was concentrated in vacuo to give the title compound (20 g) as a yellow solid. ¹H NMR (400 MHz, CDCl₃-d) δ=11.37 (s, 1H), 10.19 (s, 1H), 7.56 (dd, J=8.0, 8.8 Hz, 1H), 6.65 (d, J=9.2 Hz, 1H).

### Step 3—5-Bromo-4-fluorobenzofuran-2-carboxylic acid

To a solution of 3-bromo-2-fluoro-6-hydroxybenzaldehyde (2 g, 9 mmol) in DMF (30 mL) was added K₂CO₃ (2.52 g, 18.3 mmol) and methyl 2-bromoacetate (2.1 g, 13.7 mmol, CAS #96-32-2). The mixture was then stirred at 120° C. for 12 hrs. On completion, the reaction mixture was diluted with water (50 mL) and adjusted to pH=3 with HCl (1 M), then extracted by ethyl acetate (3×50 mL). The combined organic layers were washed with brine (2×50 mL) and dried over anhydrous sodium sulfate, filtered and the filtrate was concentrated in vacuo to give the title compound (2 g) as a brown solid. LC-MS (ESI⁺) m/z 434.0 (M+H)⁺; ¹H NMR (400 MHz, DMSO-d₆) δ=14.60-12.94 (m, 1H), 7.76-7.68 (m, 2H), 7.56 (d, J=8.8 Hz, 1H).

### 5-Bromo-4-fluoro-N,N-dimethylbenzofuran-2-carboxamide (Intermediate BW)

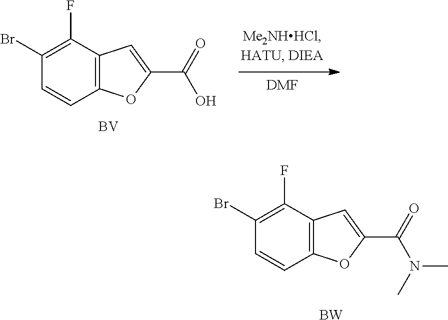

To a solution of 5-bromo-4-fluorobenzofuran-2-carboxylic acid (2 g, 8 mmol, Intermediate BV) in DMF (15 mL) was added DIEA (3.99 g, 30.9 mmol), HATU (4.40 g, 11.6 mmol) and N-methylmethanamine hydrochloride (944 mg, 11.6 mmol). The mixture was then stirred at 25° C. for 2 hrs under N₂ atmosphere. On completion, the reaction mixture was quenched with water (30 mL) and extracted with ethyl acetate (3×30 mL). The combined organic layers were washed with brine (2×30 mL) and dried over anhydrous sodium sulfate, filtered and the filtrate was concentrated in vacuo to give the crude residue. The residue was purified by column chromatography (SiO₂, Petroleum ether/Ethyl acetate=1/0 to 1/1) to give the title compound (1.8 g, 80% yield) as a white solid. LC-MS (ESI⁺) m/z 285.9 (M+H)⁺; ¹H NMR (400 MHz, CDCl₃-d) δ=7.44 (dd, J=6.4, 8.8 Hz, 1H), 7.26 (s, 1H), 7.21-7.16 (m, 1H), 3.30-3.05 (m, 6H).

### 4-Fluoro-N,N-dimethyl-5-(1,2,5,6-tetrahydropyridin-3-yl)benzofuran-2-carboxamide (Intermediate BX)

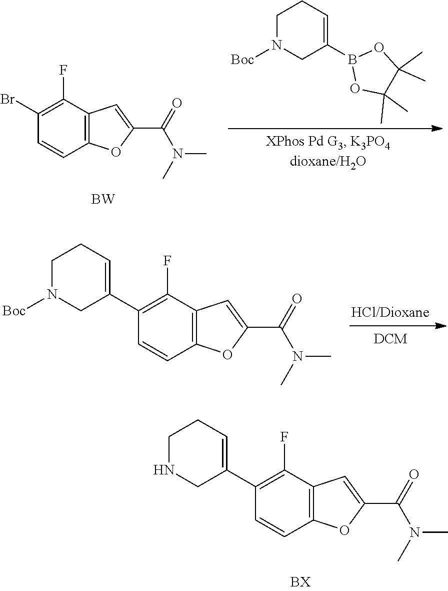

### Step 1—Tert-butyl 5-(2-(dimethylcarbamoyl)-4-fluorobenzofuran-5-yl)-3,6-dihydropyridine-1(2H)-carboxylate

A mixture of 5-bromo-4-fluoro-N,N-dimethylbenzofuran-2-carboxamide (1.7 g, 5.9 mmol, Intermediate BW), tert-butyl-5-(4,4,5,5-tetramethyl-1,3,2-dioxaborolan-2-yl)-3,6-dihydropyridine-1(2H)-carboxylate (2.76 g, 8.91 mmol, CAS #885693-20-9), XPhos Pd G₃ (503 mg, 594 μmol), and K₃PO₄ (3.78 g, 17.8 mmol) in dioxane (20 mL) and H₂O (4 mL) was degassed and purged with N₂ three times. Then the mixture was stirred at 80° C. for 2 hrs under N₂ atmosphere. On completion, the reaction mixture was diluted with water (100 mL) and extracted with ethyl acetate (3×80 mL). The combined organic layers were washed with brine (2×60 mL) and dried over anhydrous sodium sulfate, filtered and the filtrate was concentrated in vacuo to give the crude residue. The residue was purified by column chromatography (SiO₂, Petroleum ether/Ethyl acetate=1/0 to 1/1) to give the title compound (2 g, 85% yield) as a brown solid. LC-MS (ESI⁺) m/z 389.1 (M+H)⁺.

### Step 2—4-Fluoro-N,N-dimethyl-5-(1,2,5,6-tetrahydropyridin-3-yl)benzofuran-2-carboxamide

To a solution of tert-butyl 5-(2-(dimethylcarbamoyl)-4-fluorobenzofuran-5-yl)-3,6-dihydropyridine-1(2H)-carboxylate (1.9 g, 4.9 mmol) in DCM (30 mL) was added HCl/dioxane (4 M, 5.18 mL). The mixture was then stirred at 25° C. for 4 hrs. On completion, the reaction mixture was concentrated in vacuo to give the title compound (1.8 g) as a yellow solid. LC-MS (ESI⁺) m/z 289.1 (M+H)⁺.

### 2-(Tert-butoxycarbonyl)-1,2,3,4-tetrahydroisoquinoline-3-carboxylic acid (CAS #151838-62-9) (Intermediate BY)

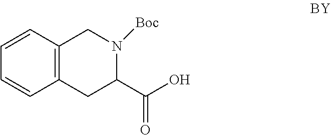 [SMILES: CC(C)(C)OC(=O)N1Cc2ccccc2CC1C(=O)O]

### Tert-butyl (3-methoxy-4-(4,4,5,5-tetramethyl-1,3,2-dioxaborolan-2-yl)phenyl)(methyl)carbamate (Intermediate BZ)

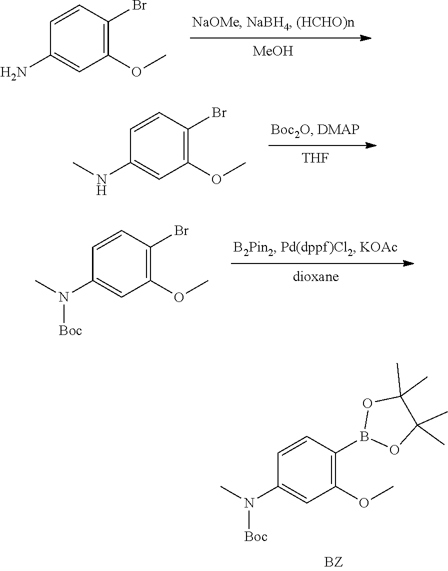

### Step 1—4-Bromo-3-methoxy-N-methyl-aniline

To a 4-bromo-3-methoxy-aniline (1.00 g, 4.95 mmol, CAS #19056-40-7) and (HCHO)n (1.44 g, 49.4 mmol) in MeOH (25.0 mL) was added NaOMe (5.4 M, 4.58 mL) under 0° C. Then the reaction mixture was stirred at 85° C. for 2 h. After that, it was cooled to 0° C. and NaBH₄ (749 mg, 19.8 mmol) was added and the mixture was stirred for 30 mins. Then the mixture was stirred at 85° C. for 3 h. On completion, the mixture was cooled to 0° C., and the reaction mixture was quenched by addition of NH₄Cl aq. (10 mL) and extracted with ethyl acetate (15 mL×2), dried over sodium sulfate, filtered and concentrated under reduced pressure. The residue was purified by silica gel column chromatography (petroleum ether˜petroleum ether/ethyl acetate=10/1) to get the title compound (540 mg, 46% yield) as a white solid. ¹H NMR (400 MHz, DMSO-d₆) δ=7.17 (d, J=8.4 Hz, 1H), 6.24 (d, J=2.4 Hz, 1H), 6.07 (dd, J=2.4, 8.6 Hz, 1H), 5.85 (d, J=4.8 Hz, 1H), 3.76 (s, 3H), 2.66 (d, J=4.8 Hz, 3H).

### Step 2—Tert-butyl N-(4-bromo-3-methoxy-phenyl)-N-methyl-carbamate

To a solution of 4-bromo-3-methoxy-N-methyl-aniline (415 mg, 1.92 mmol) in THF (4.0 mL) was added Boc₂O (838 mg, 3.84 mmol, 882 uL) and DMAP (281 mg, 2.30 mmol). Then the mixture was stirred at 25° C. for 6 h. On completion, the mixture was diluted with water (15 mL) and extracted with ethyl acetate (15 mL×2), dried over sodium sulfate, filtered and concentrated under reduced pressure to get the title compound (530 mg) as a white solid. ¹H NMR (400 MHz, DMSO-d₆) δ=7.48 (d, J=8.0 Hz, 1H), 6.91-6.76 (m, 2H), 3.72 (s, 3H), 3.19 (s, 3H), 1.40 (s, 9H), 1.26 (s, 12H).

### Step 3—Tert-butyl N-[3-methoxy-4-(4,4,5,5-tetramethyl-1,3,2-dioxaborolan-2-yl)phenyl]-N-methyl-carbamate

A mixture of tert-butyl N-(4-bromo-3-methoxy-phenyl)-N-methyl-carbamate (530 mg, 1.68 mmol), Pd(dppf)Cl₂ (122 mg, 168 umol), KOAc (493 mg, 5.03 mmol) and 4,4,5,5-tetramethyl-2-(4,4,5,5-tetramethyl-1,3,2-dioxaborolan-2-yl)-1,3,2-dioxaborolane (1.28 g, 5.03 mmol) in 1,4-dioxane (5.0 mL) was degassed and purged with N₂ three times. Then the mixture was stirred at 90° C. for 2 h under N₂ atmosphere. On completion, the mixture was concentrated under reduced pressure using a rotary evaporator. The residue was purified by silica gel column chromatography (petroleum ether/ethyl acetate=10/1˜1/1) to give the title compound (420 mg, 69% yield) as a white solid. LC-MS (ESI⁺) m/z 308.4 (M+H)/2⁺.

### Methyl 6-bromo-4-chloro-1H-indole-2-carboxylate (Intermediate CA)

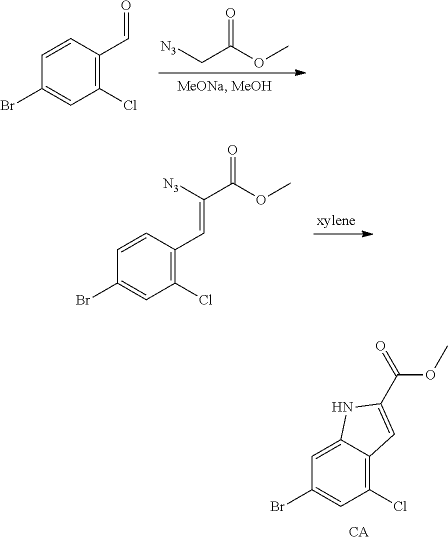

### Step 1—Methyl-2-azido-3-(4-bromo-2-chlorophenyl)acrylate

To a solution of 4-bromo-2-chlorobenzaldehyde (20.0 g, 91.1 mmol, CAS #158435-41-7) in MeOH (200 mL) was added sodium methylate (5.4 M, 67.5 mL, 364 mmol) at 25° C. Then the mixture was cool down to −40° C. under N₂ atmosphere and methyl 2-azidoacetate (42.0 g, 365 mmol, CAS #1816-92-8) was added dropwise at this temperature. The mixture allowed to warm up to 25° C. and the mixture was stirred for 12 hrs. On completion, the reaction mixture was poured into ice water (40 mL) and stirred at 0-20° C. for 1 h. The mixture was then filtered to give the filter cake as the title compound (20.0 g, 69% yield) as a greyish-green solid.

### Step 2—Methyl 6-bromo-4-chloro-1H-indole-2-carboxylate

A mixture of methyl-2-azido-3-(4-bromo-2-chlorophenyl)acrylate (20.0 g, 63.2 mmol) in xylene (400 mL) was heated to 140° C. for 12 h under N₂ atmosphere. On completion, the reaction mixture was cooled to 0° C. and stirred at 2 h. Then the mixture was filtered and the filter cake was dried to give the title compound (5.40 g, 29% yield) as a gray solid. ¹H NMR (400 MHz, DMSO-d₆) δ=12.91-11.86 (m, 1H), 7.59 (s, 1H), 7.38 (s, 1H), 7.12 (s, 1H), 3.90 (s, 3H).

### Methyl 4-chloro-6-(1,2,5,6-tetrahydropyridin-3-yl)-1H-indole-2-carboxylate (Intermediate CB)

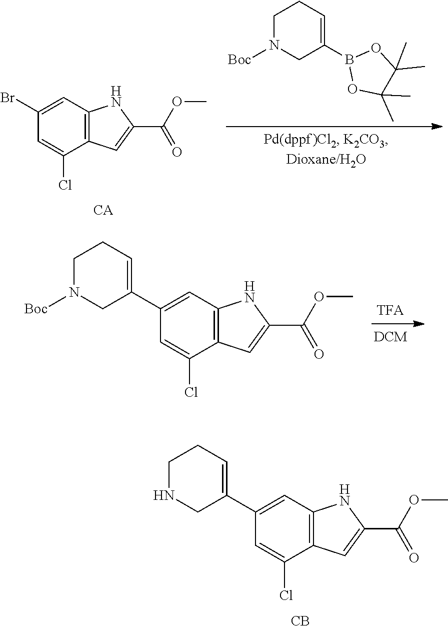

### Step 1—Methyl 6-(1-(tert-butoxycarbonyl)-1,2,5,6-tetrahydropyridin-3-yl)-4-chloro-1H-indole-2-carboxylate

A mixture of methyl 6-bromo-4-chloro-1H-indole-2-carboxylate (2 g, 7 mmol, Intermediate CA), tert-butyl 5-(4,4,5,5-tetramethyl-1,3,2-dioxaborolan-2-yl)-3,6-dihydropyridine-1(2H)-carboxylate (2.14 g, 6.93 mmol, CAS #885693-20-9), Pd(dppf)Cl₂ (507 mg, 693 μmol), and K₂CO₃ (2.87 g, 20.8 mmol) in dioxane (20 mL) and H₂O (2 mL) was degassed and purged with N₂ three times. Then the mixture was stirred at 80° C. for 12 hrs under N₂ atmosphere. On completion, the reaction mixture was quenched with water (30 mL) and extracted with dichloromethane (3×30 mL). The combined organic layers were washed with aqueous NaCl (30 mL) and dried over anhydrous sodium sulfate, filtered and the filtrate was concentrated in vacuo to give the crude residue. The residue was purified by column chromatography (SiO₂, Petroleum ether:Ethyl acetate=3/1) to give the title compound (2.5 g, 91% yield) as a yellow solid. LC-MS (ESI⁺) m/z 335.0 (M+H)⁺.

### Step 2—Methyl 4-chloro-6-(1,2,5,6-tetrahydropyridin-3-yl)-1H-indole-2-carboxylate

To a solution of methyl 6-(1-(tert-butoxycarbonyl)-1,2,5,6-tetrahydropyridin-3-yl)-4-chloro-1H-indole-2-carboxylate (1 g, 2.56 mmol) in DCM (10 mL) was added TFA (1.54 g, 13.5 mmol). The mixture was stirred at 25° C. for 4 hrs. On completion, the reaction mixture was concentrated under reduced pressure to remove solvent to give the title compound (1 g, TFA) as a red solid. LC-MS (ESI⁺) m/z 290.9 (M+H)⁺.

### Methyl 4-chloro-6-(1-(1,2,3,4-tetrahydroisoquinoline-3-carbonyl)-1,2,5,6-tetrahydropyridin-3-yl)-1H-indole-2-carboxylate (Intermediate CC)

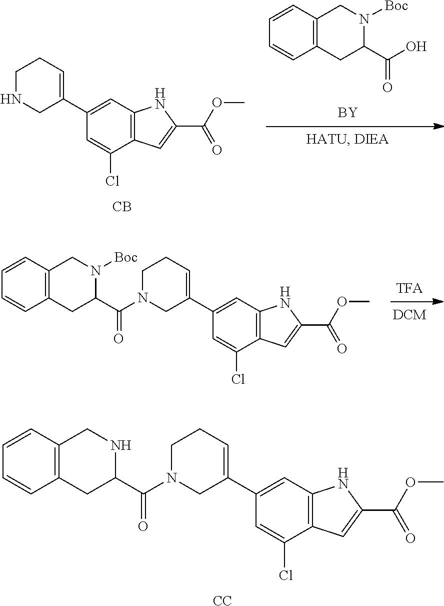

### Step 1—Tert-butyl 3-(5-(4-chloro-2-(methoxycarbonyl)-1H-indol-6-yl)-1,2,3,6-tetrahydropyridine-1-carbonyl)-3,4-dihydroisoquinoline-2(1H)-carboxylate

To a solution of 2-(tert-butoxycarbonyl)-1,2,3,4-tetrahydroisoquinoline-3-carboxylic acid (1.03 g, 3.71 mmol, Intermediate BY) in DMF (5 mL) was added HATU (1.83 g, 4.82 mmol), DIEA (3.83 g, 29.6 mmol, 5.16 mL), and methyl 4-chloro-6-(1,2,5,6-tetrahydropyridin-3-yl)-1H-indole-2-carboxylate (1.5 g, 3.71 mmol, TFA, Intermediate CB). The mixture was then stirred at 25° C. for 2 hrs. On completion, the reaction mixture was quenched with water (30 mL) and extracted with ethyl acetate (3×30 mL). The combined organic layers were washed with aqueous NaCl (30 mL) and dried over anhydrous sodium sulfate, filtered and the filtrate was concentrated in vacuo to give the title compound (1.9 g) as a brown solid. LC-MS (ESI⁺) m/z 450.3 (M−99)⁺.

### Step 2—Methyl 4-chloro-6-(1-(1,2,3,4-tetrahydroisoquinoline-3-carbonyl)-1,2,5,6-tetrahydropyridin-3-yl)-1H-indole-2-carboxylate

To a solution of tert-butyl 3-(5-(4-chloro-2-(methoxycarbonyl)-1H-indol-6-yl)-1,2,3,6-tetrahydropyridine-1-carbonyl)-3,4-dihydroisoquinoline-2(1H)-carboxylate (1.9 g, 3.5 mmol) in DCM (10 mL) was added TFA (1.54 g, 13.5 mmol, 1 mL). The mixture was then stirred at 25° C. for 2 hrs. On completion, the reaction mixture was concentrated under reduced pressure to remove solvent to give the title compound (1.5 g, TFA) as a pink solid. LC-MS (ESI⁺) m/z 450.3 (M+H)⁺.

### 4-Chloro-6-(1-(2-formyl-1,2,3,4-tetrahydroisoquinoline-3-carbonyl)-1,2,5,6-tetrahydropyridin-3-yl)-1H-indole-2-carboxylic acid (Intermediate CD)

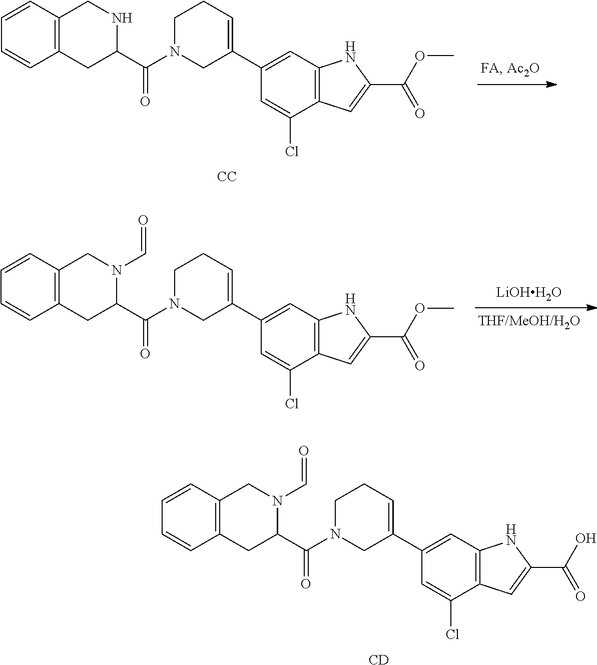

### Step 1—Methyl 4-chloro-6-(1-(2-formyl-1,2,3,4-tetrahydroisoquinoline-3-carbonyl)-1,2,5,6-tetrahydropyridin-3-yl)-1H-indole-2-carboxylate

To a solution of methyl 4-chloro-6-(1-(1,2,3,4-tetrahydroisoquinoline-3-carbonyl)-1,2,5,6-tetrahydropyridin-3-yl)-1H-indole-2-carboxylate (1.5 g, 3.3 mmol, Intermediate CC) in HCOOH (10 mL) was added Ac₂O (10.9 g, 106 mmol). The mixture was then stirred at 25° C. for 2 hrs. On completion, the reaction mixture was filtered and the filter cake was dried in vacuo to give the title compound (1.9 g) as a white solid. LC-MS (ESI⁺) m/z 478.3 (M+H)⁺.

### Step 2—4-Chloro-6-(1-(2-formyl-1,2,3,4-tetrahydroisoquinoline-3-carbonyl)-1,2,5,6-tetrahydropyridin-3-yl)-1H-indole-2-carboxylic acid

A mixture of methyl 4-chloro-6-(1-(2-formyl-1,2,3,4-tetrahydroisoquinoline-3-carbonyl)-1,2,5,6-tetrahydropyridin-3-yl)-1H-indole-2-carboxylate (1.2 g, 2.5 mmol), LiOH·H₂O (527 mg, 12.6 mmol) in THF (8 mL), MeOH (2 mL) and H₂O (2 mL). Then the mixture was stirred at 25° C. for 2 hrs. On completion, the reaction mixture was quenched with water (30 mL) and extracted with ethyl acetate (3×30 mL). The combined organic layers were washed with aqueous NaCl (30 mL) and dried over anhydrous sodium sulfate, filtered and the filtrate was concentrated in vacuo to give the title compound (1 g) as a yellow solid. LC-MS (ESI⁺) m/z 464.3 (M+H)⁺.

### 4-Chloro-6-(1-(2-formyl-1,2,3,4-tetrahydroisoquinoline-3-carbonyl)-1,2,5,6-tetrahydropyridin-3-yl)-N,N-dimethyl-1H-indole-2-carboxamide (Intermediate CE)

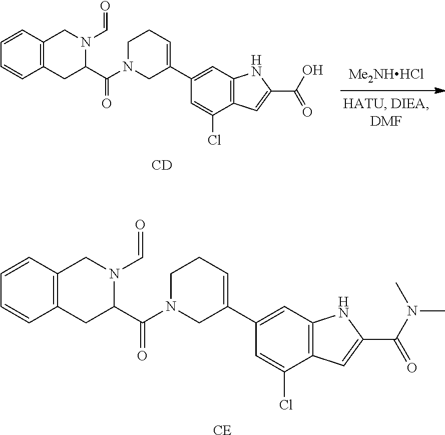 [SMILES: "Me2NH·HCl" ; O=CN1Cc2ccccc2CC1C(=O)N1CCC=C(c2cc(Cl)c3cc(C(=O)O)[nH]c3c2)C1 ; "CD" ; CN(C)C(=O)c1cc2c(Cl)cc(C3=CCCN(C(=O)C4Cc5ccccc5CN4C=O)C3)cc2[nH]1 ; "CE"]

To a solution of 4-chloro-6-(1-(2-formyl-1,2,3,4-tetrahydroisoquinoline-3-carbonyl)-1,2,5,6-tetrahydropyridin-3-yl)-1H-indole-2-carboxylic acid (1 g, 2 mmol, Intermediate CD) in DMF (5 mL) was added HATU (984 mg, 2.59 mmol), DIEA (2.23 g, 17.2 mmol), and N-methylmethanamine hydrochloride (211 mg, 2.59 mmol, CAS #506-59-2). The mixture was then stirred at 25° C. for 12 hrs. On completion, the reaction mixture was concentrated under reduced pressure to remove solvent. The crude residue was purified by reversed-phase HPLC (0.1% FA condition) to give the title compound (270 mg, 25% yield) as a white solid. LC-MS (ESI⁺) m/z 491.3 (M+H).

### 1-(Benzo[d][1,3]dioxol-5-yl)piperazine (Intermediate CF)

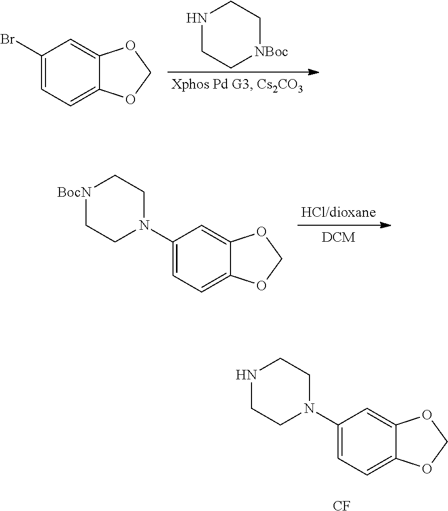 [SMILES: Brc1ccc2c(c1)OCO2 ; CC(C)(C)OC(=O)N1CCNCC1 ; CC(C)(C)OC(=O)N1CCN(c2ccc3c(c2)OCO3)CC1 ; c1cc2c(cc1N1CCNCC1)OCO2 ; "CF"]

### Step 1—Tert-butyl 4-(benzo[d][1,3]dioxol-5-yl)piperazine-1-carboxylate

A mixture of 5-bromobenzo[d][1,3]dioxole (500 mg, 3 mmol, CAS #2635-13-4), tert-butyl piperazine-1-carboxylate (554 mg, 2.49 mmo, CAS #57260-71-6), Cs₂CO₃ (2.43 g, 7.46 mmol), and Xant Phos Pd G3 (236 mg, 249 μmol) in dioxane (10 mL) was degassed and purged with N₂ three times. Then the mixture was stirred at 100° C. for 24 hrs under N₂ atmosphere. On completion, the reaction mixture was quenched with water (30 mL) and extracted with ethyl acetate (3×30 mL). The combined organic layers were washed with aqueous NaCl (30 mL) and dried over anhydrous sodium sulfate, filtered and the filtrate was concentrated in vacuo to give the crude residue. The residue was purified by column chromatography (SiO₂, Petroleum ether:Ethyl acetate=3/1) to give the title compound (500 mg, 64% yield) as a white solid. LC-MS (ESI⁺) m/z 307.2 (M+H)⁺.

### Step 2—1-(Benzo[d][1,3]dioxol-5-yl)piperazine

To a solution of tert-butyl 4-(benzo[d][1,3]dioxol-5-yl)piperazine-1-carboxylate (250 mg, 816 μmol) in DCM (2 mL) was added HCl/dioxane (4 M, 0.2 mL). The mixture was then stirred at 25° C. for 12 hrs. On completion, the reaction mixture was concentrated under reduced pressure to remove solvent to give the title compound (230 mg, HCl) as a white solid. LC-MS (ESI⁺) m/z 207.3 (M+H)⁺.

### (3-Methoxypyridin-4-yl)boronic acid (CAS #1008506-24-8) (Intermediate CG)

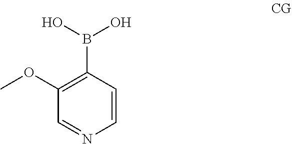

### 1-(5-(4,4,5,5-tetramethyl-1,3,2-dioxaborolan-2-yl)-3,6-dihydropyridin-1(2H)-yl)ethan-1-one (Intermediate CH)

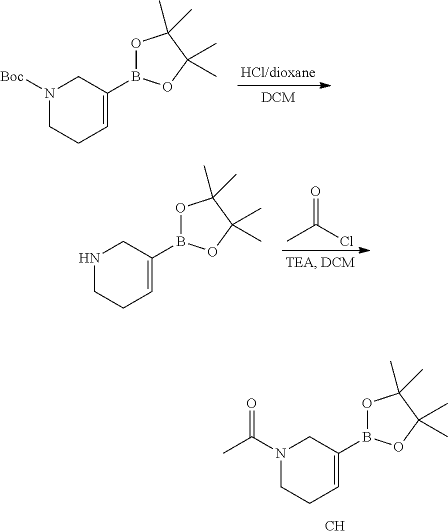

### Step 1—5-(4,4,5,5-Tetramethyl-1,3,2-dioxaborolan-2-yl)-1,2,3,6-tetrahydropyridine

To a solution of tert-butyl 5-(4,4,5,5-tetramethyl-1,3,2-dioxaborolan-2-yl)-3,6-dihydropyridine-1(2H)-carboxylate (30.0 g, 97.0 mmol, CAS #885693-20-9) in DCM (150 mL) was added HCl/dioxane (4 M, 150 mL), then the mixture was stirred at 20° C. for 1 h. After completion, the reaction mixture was concentrated under pressure to give the title compound (24.0 g, HCl) as a yellow solid. LC-MS (ESI⁺) m/z 210.3 (M+H)⁺.

### Step 2—1-(5-(4,4,5,5-Tetramethyl-1,3,2-dioxaborolan-2-yl)-3,6-dihydropyridin-1(2H)-yl)ethan-1-one

To a solution of 5-(4,4,5,5-tetramethyl-1,3,2-dioxaborolan-2-yl)-1,2,3,6-tetrahydropyridine (24.0 g, 97.7 mmol, HCl) in DCM (300 mL) was added DIEA (85.0 mL, 489 mmol) and acetyl chloride (10.5 mL, 147 mmol, CAS #75-36-5), then the mixture was stirred at 20° C. for 2 h. After completion, the reaction mixture was quenched by addition of water (500 mL) at 0° C., and then extracted with DCM (200 mL×2). The combined organic layers were washed with brine (500 mL), dried over anhydrous Na₂SO₄, filtered and concentrated under reduced pressure to give a residue. The residue was triturated with EtOAc/PE=2:1 to afford the title compound (15.0 g, 59.7 mmol, 61.1% yield) as a yellow solid. ¹H NMR (400 MHz, DMSO-d₆) δ ppm 6.64-6.47 (m, 1H), 3.93 (s, 2H), 3.46 (td, J=5.6, 11.6 Hz, 2H), 2.24-2.09 (m, 2H), 2.00 (s, 3H), 1.21 (d, J=3.6 Hz, 12H).

### 6-(1-Acetyl-1,2,5,6-tetrahydropyridin-3-yl)-4-chloro-7-fluoro-N,N-dimethyl-1H-indole-2-carboxamide (Intermediate CI)

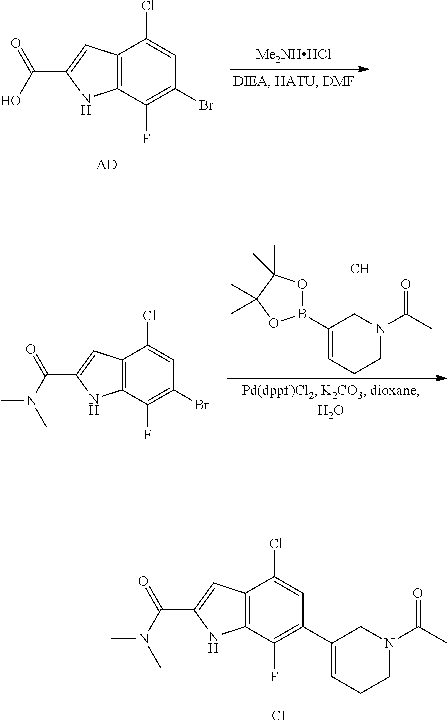

### Step 1—6-Bromo-4-chloro-7-fluoro-N,N-dimethyl-1H-indole-2-carboxamide

To a solution of 6-bromo-4-chloro-7-fluoro-1H-indole-2-carboxylic acid (8.9 g, 30.4 mmol, Intermediate AD) in DMF (100 mL) was added N-methylmethanamine (4.96 g, 60.9 mmol, 5.58 mL, HCl), DIEA (11.8 g, 91.3 mmol, 15.9 mL) and HATU (12.7 g, 33.5 mmol). The reaction was then stirred at 25° C. for 1 hr. On completion, the reaction mixture was quenched with water (300 mL) and mixture was filtered and the filter cake was concentrated in vacuo to give the title compound (7.76 g) as a white solid. LC-MS (ESI⁺) m/z 319.0. (M+H)⁺.

### Step 2—6-(1-Acetyl-1,2,5,6-tetrahydropyridin-3-yl)-4-chloro-7-fluoro-N,N-dimethyl-1H-indole-2-carboxamide

To a solution of 6-bromo-4-chloro-7-fluoro-N,N-dimethyl-1H-indole-2-carboxamide (6.74 g, 21.1 mmol) in dioxane (80 mL) and H₂O (20 mL) was added 1-(5-(4,4,5,5-tetramethyl-1,3,2-dioxaborolan-2-yl)-3,6-dihydropyridin-1(2H)-yl)ethan-1-one (5.3 g, 21.1 mmol, Intermediate CH), K₂CO₃ (8.75 g, 63.3 mmol) and Pd(dppf)Cl₂ (1.54 g, 2.11 mmol). The reaction was then stirred at 80° C. for 2 hrs under N₂ atmosphere. On completion, the reaction mixture was quenched with water (100 mL) and extracted with ethyl acetate (3×200 mL). The combined organic layers were washed with brine (200 mL) and dried over anhydrous sodium sulfate, filtered and the filtrate was concentrated in vacuo to give the title compound (8 g) as a brown oil. LC-MS (ESI⁺) m/z 364.2. (M+H)⁺.

### 1-Cyclobutylpiperazine (CAS #132800-13-6) (Intermediate CJ)

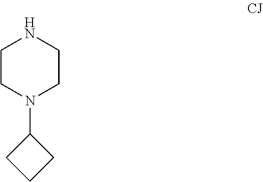

### 3-(Piperazin-1-yl)phenol (CAS #59817-32-2) (Intermediate CK)

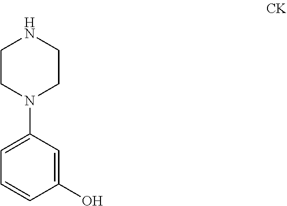 [SMILES: Oc1cccc(N2CCNCC2)c1]

### 3-Methoxy-2-(piperidin-4-yl)pyridine (Intermediate CL)

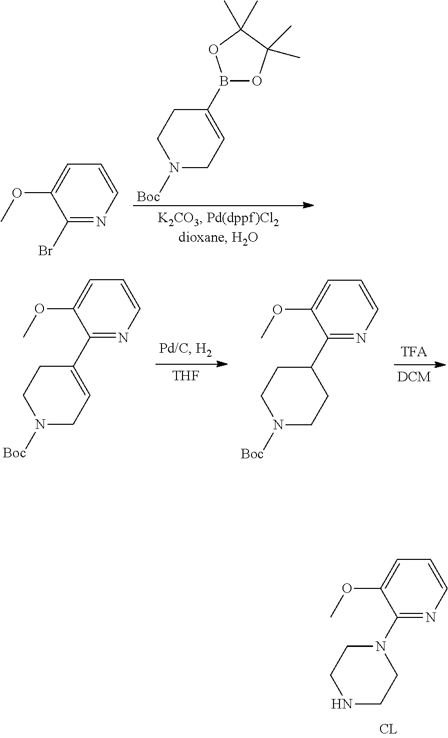

### Step 1—Tert-butyl 3-methoxy-3′,6′-dihydro-[2,4′-bipyridine]-1′(2′H)-carboxylate

A mixture of 2-bromo-3-methoxypyridine (2 g, 10 mmol, CAS #24100-18-3), tert-butyl 4-(4,4,5,5-tetramethyl-1,3,2-dioxaborolan-2-yl)-3,6-dihydropyridine-1(2H)-carboxylate (3.95 g, 12.7 mmol, CAS #286961-14-6), K₂CO₃ (4.41 g, 31.9 mmol), and Pd(dppf)Cl₂ (778 mg, 1.06 mmol) in dioxane (20 mL) and H₂O (2 mL) was degassed and purged with N₂ three times. Then the mixture was stirred at 80° C. for 12 hrs under N₂ atmosphere. On completion, the mixture was diluted with water (10 mL) and extracted with dichloromethane (2×20 mL). The combined organic layers were washed with brine (40 mL) and dried over anhydrous sodium sulfate, filtered and the filtrate was concentrated in vacuo to give the crude residue. The crude residue was purified by column chromatography (SiO₂, Petroleum ether:Ethyl acetate=1/0 to 7/3) to give the title compound (1.3 g, 42% yield) as a colorless oil. LC-MS (ESI⁺) m/z 291.1 (M+H)⁺.

### Step 2—Tert-butyl 4-(3-methoxypyridin-2-yl)piperidine-1-carboxylate

To a solution of tert-butyl 3-methoxy-3′,6′-dihydro-[2,4′-bipyridine]-1′(2′H)-carboxylate (1 g, 3 mmol) in THF (10 mL) was added Pd/C (10 wt %, 200 mg) under N₂ atmosphere. The suspension was degassed and purged with H₂ three times. Then the mixture was stirred under H₂ (15 psi) at 25° C. for 6 hrs. On completion, the mixture was filtered and the filtrate was concentrated in vacuo to give the title compound (1 g) as colorless oil. LC-MS (ESI⁺) m/z 293.2 (M+H)⁺.

### Step 3—3-Methoxy-2-(piperidin-4-yl)pyridine

To a solution of tert-butyl 4-(3-methoxypyridin-2-yl)piperidine-1-carboxylate (1 g, 3 mmol) in DCM (10 mL) was added TFA (1 mL). The mixture was stirred at 25° C. for 12 hrs. On completion, the mixture was concentrated in vacuo to give the title compound (500 mg, TFA) as white solid. LC-MS (ESI⁺) m/z 193.2 (M+H)⁺.

### 5-(Piperazin-1-yl)-1H-pyrrolo[2,3-b]pyridine-6-carbonitrile (Intermediate CM), 7-(piperazin-1-yl)-1H-pyrrolo[2,3-c]pyridine (Intermediate CN), 4-fluoro-2-(piperazin-1-yl)benzonitrile (Intermediate CO), 1-(6-ethylpyridin-2-yl)piperazine (Intermediate CP), and 4-(6-(piperazin-1-yl)pyridin-2-yl)morpholine (Intermediate CQ)

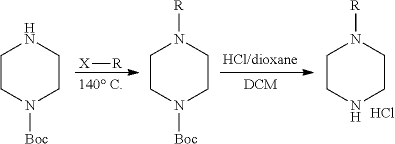

R:

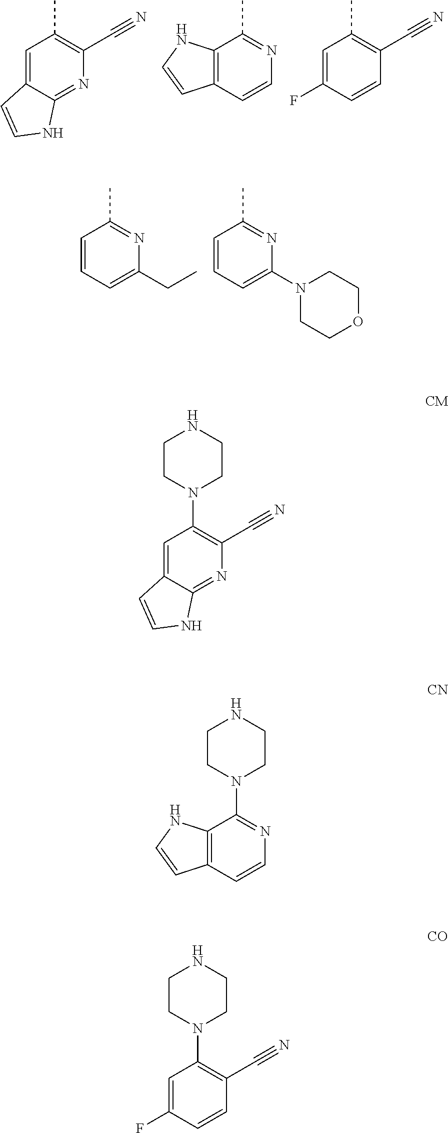

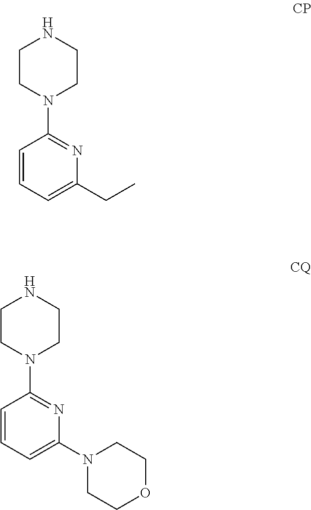 [SMILES: CCc1cccc(N2CCNCC2)n1 ; c1cc(N2CCNCC2)nc(N2CCOCC2)c1]

### Step 1

Tert-butyl piperazine-1-carboxylate (10.0 eq) and compounds R—X (1.0 eq) were stirred at 140° C. for 16 hours under N₂. On completion, the mixture was diluted with EtOAc (10 mL) and washed with H₂O (5 mL) and brine (5 mL). The organic phase was evaporated to obtain the crude product. The crude product was purified by prep-HPLC (neutral condition) and evaporated to obtain the title compounds.

### Step 2

To a mixture of the Boc protected amines (0.1 g, 1.0 eq) in DCM (2 mL) was added HCl/Dioxane (4N, 4 mL) at 25° C. The mixture was then stirred at 25° C. for 4 hrs. On completion, the reaction mixture was concentrated under reduced pressure to give the title compounds.

### 1-(4-Chloro-3-methoxypyridin-2-yl)piperazine (Intermediate CR)

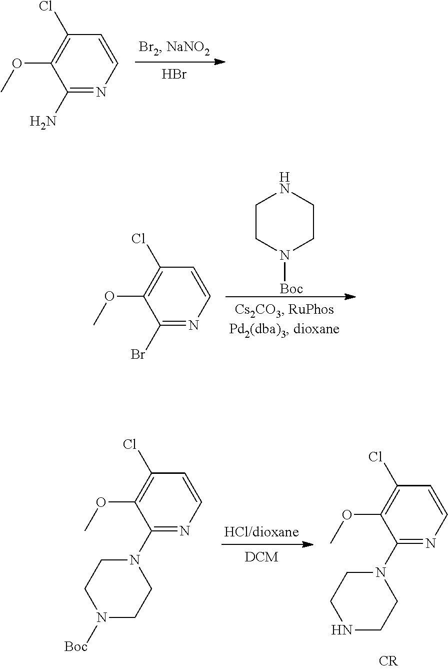

### Step 1—2-Bromo-4-chloro-3-methoxypyridine

To a solution of 4-chloro-3-methoxy-pyridin-2-amine (1 g, 6 mmol, CAS #1261452-97-4) in HBr (10 mL) was added Br₂ (3.02 g, 18.9 mmol, 974 μL) and NaNO₂ (1.09 g, 15.7 mmol). The mixture was then stirred at 0° C. for 2 h. On completion, the reaction mixture was quenched with water (30 mL) and extracted with ethyl acetate (3×20 mL). The combined organic layers were washed with brine (25 mL) and dried over anhydrous sodium sulfate, filtered and the filtrate was concentrated in vacuo to give the title compound (700 mg, 50% yield) as a yellow oil. LC-MS (ESI⁺) m/z 223.8 (M+H)⁺.

### Step 2—Tert-butyl 4-(4-chloro-3-methoxypyridin-2-yl)piperazine-1-carboxylate

To a solution of 2-bromo-4-chloro-3-methoxy-pyridine (650 mg, 2.90 mmol) in dioxane (5 mL) was added RuPhos (272 mg, 584 μmol) and Cs₂CO₃ (2.86 g, 8.76 mmol), Pd₂(dba)₃ (267 mg, 292 mol) and tert-butyl piperazine-1-carboxylate (653 mg, 3.5 mmol). The mixture was then stirred at 100° C. for 4 h. On completion, the reaction mixture was diluted with H₂O (10 mL) and extracted with EA (10 mL). The combined organic layers were washed with H₂O (20 mL), dried over Na₂SO₄, filtered and concentrated under reduced pressure to give a residue. The residue was purified by column chromatography (SiO₂, Petroleum ether/Ethyl acetate=10/1 to 5/1) to give the title compound (180 mg, 20% yield) as a yellow oil. LC-MS (ESI⁺) m/z 328.0 (M+H)⁺.

### Step 3—1-(4-Chloro-3-methoxypyridin-2-yl)piperazine

To a solution of tert-butyl 4-(4-chloro-3-methoxy-2-pyridyl)piperazine-1-carboxylate (180 mg, 549 μmol) in DCM (0.5 mL) was added HCl/dioxane (2 mL). The mixture was then stirred at 25° C. for 0.5 h. On completion, the reaction mixture was concentrated under reduced pressure to give the title compound (60 mg) as a yellow solid. LC-MS (ESI⁺) m/z 228.0 (M+H)⁺.

### 1-(4-Fluoro-3-methoxypyridin-2-yl)piperazine (Intermediate CS)

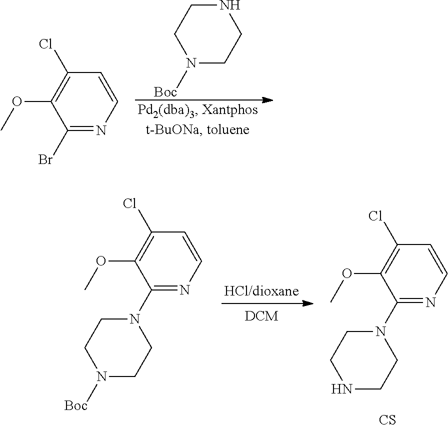

### Step 1—Ten-butyl 4-(4-fluoro-3-methoxypyridin-2-yl)piperazine-1-carboxylate

A mixture of 2-bromo-4-fluoro-3-methoxypyridine (407 mg, 2.18 mmol, CAS #1256819-71-2), tert-butyl piperazine-1-carboxylate (450 mg, 2.18 mmol, CAS #57260-71-6), t-BuONa (1.05 g, 10.9 mmol), Xantphos (126 mg, 218 μmol) and Pd₂(dba)₃ (200 mg, 218 μmol) in toluene (3 mL) was degassed and purged with N₂ three times. Then the mixture was stirred at 100° C. for 2 hrs under N₂ atmosphere. On completion, the reaction mixture was concentrated under reduced pressure to give a residue. The residue was purified by column chromatography (SiO₂, Petroleum ether:Ethyl acetate=10/1 to 5/1) to give the title compound (0.5 g, 66% yield) as a white solid. LC-MS (ESI⁺) m/z 312.1 (M+H)⁺.

### Step 2—1-(4-Fluoro-3-methoxypyridin-2-yl)piperazine

To a solution of tert-butyl 4-(4-fluoro-3-methoxypyridin-2-yl)piperazine-1-carboxylate (0.5 g, 1.6 mmol) in DCM (6 mL) was added HCl/dioxane (8 M, 2 mL). The mixture was then stirred at 25° C. for 0.5 hrs. On completion, the reaction mixture was concentrated under reduced pressure to give the title compound (0.5 g, HCl) as a white solid. LC-MS (ESI⁺) m/z 212.2 (M+H)⁺.

### (2-(Trifluoromethyl)phenyl)boronic acid (CAS #1423-27-4) (Intermediate CT)

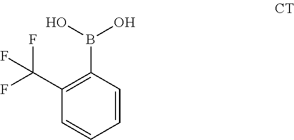 [SMILES: OB(O)c1ccccc1C(F)(F)F]

### 1-(3-(Fluoromethoxy)pyridin-2-yl)piperazine (Intermediate CU)

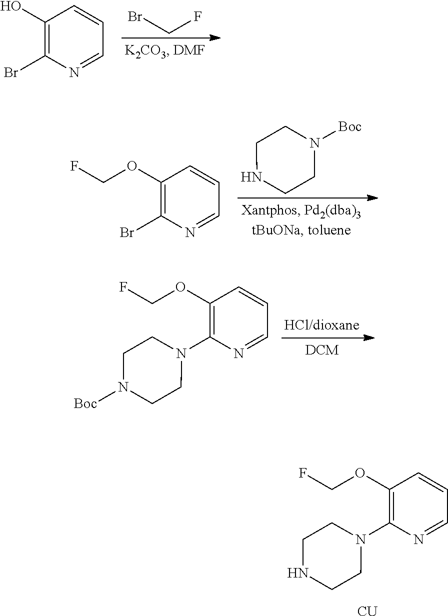

### Step 1—2-Bromo-3-(fluoromethoxy)pyridine

To a solution of bromofluoromethane (750 mg, 4.31 mmol) in DMF (10 mL) was added K₂CO₃ (1.19 g, 8.62 mmo) and bromofluoromethane (974 mg, 8.62 mmol, CAS #373-52-4). The mixture was then stirred at 25° C. for 2 hrs. On completion, the crude product was concentrated under reduced pressure to give a residue. The residue was purified by column chromatography (SiO₂, Petroleum ether/Ethyl acetate=1/0 to 4/1) to give the title compound (200 mg, 23% yield) as a white solid. LC-MS (ESI⁺) m/z 205.8 (M+H)⁺.

### Step 2—Tert-butyl 4-(3-(fluoromethoxy)pyridin-2-yl)piperazine-1-carboxylate

A mixture of 2-bromo-3-(fluoromethoxy)pyridine (180 mg, 874 μmol), tert-butyl piperazine-1-carboxylate (244.10 mg, 1.31 mmol, CAS #57260-71-6), Cs₂CO₃ (854 mg, 2.62 mmol), Pd₂(dba)₃ (80.0 mg, 87.4 μmol) and RuPhos (40.8 mg, 87.4 μmol) in dioxane (4 mL) was degassed and purged with N₂ three times. Then the mixture was stirred at 80° C. for 12 hrs under N₂ atmosphere. On completion, the crude product was concentrated under reduced pressure to give a residue. The residue was purified by column chromatography (SiO₂, Petroleum ether/Ethyl acetate=1/0 to 4/1) to give the title compound (200 mg, 23% yield) as a yellow oil. LC-MS (ESI⁺) m/z 312.0 (M+H)⁺.

### Step 3—1-(3-(Fluoromethoxy)pyridin-2-yl)piperazine

To a solution of tert-butyl 4-(3-(fluoromethoxy)pyridin-2-yl)piperazine-1-carboxylate (220 mg, 707 μmol) in DCM (1 mL) was added HCl/Dioxane (4 M, 11.0 mL). The mixture was then stirred at 25° C. for 1 hr. On completion, the reaction mixture was concentrated in vacuo to give the title compound (200 mg, HCl) as a yellow oil. LC-MS (ESI⁺) m/z 212.1 (M+H)⁺.

### 1-(3-Methoxyphenyl)piperazine (CAS #16015-71-7) (Intermediate CV)

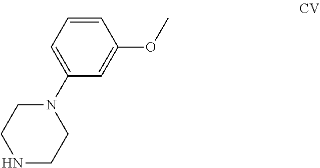

### 2-(Piperazin-1-yl)benzonitrile (CAS #111373-03-6) (Intermediate CW)

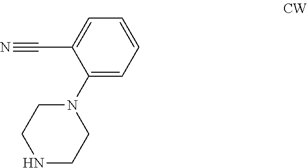 [SMILES: N#Cc1ccccc1N1CCNCC1]

### (4-Fluoro-2-methoxy-phenyl)boronic acid (CAS #624741-83-9) (Intermediate CW)

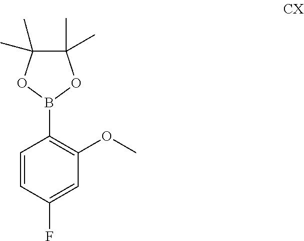

### (5-Fluoro-2-methoxy-phenyl)boronic acid (CAS #179897-94-0) (Intermediate CY)

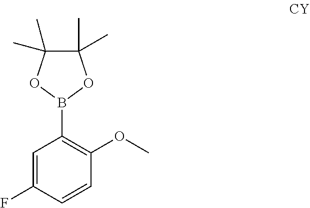 [SMILES: COc1ccc(F)cc1B1OC(C)(C)C(C)(C)O1]

### (1-Methoxynaphthalen-2-yl)boronic acid (CAS #252670-79-4) (Intermediate CZ)

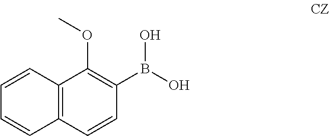

### Naphthalen-1-ylboronic acid (CAS #-13922-4-1-3) (Intermediate DA)

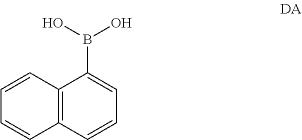 [SMILES: OB(O)c1cccc2ccccc12]

### 2,3-Dihydro-1,4-benzodioxin-5-ylboronic acid (CAS #499769-88-9) (Intermediate DB)

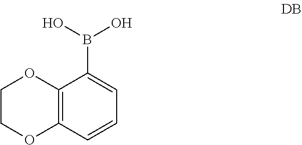 [SMILES: OB(O)c1cccc2c1OCCO2]

### (3-Fluoro-2-methoxyphenyl)boronic acid (CAS #762287-59-2) (Intermediate DC)

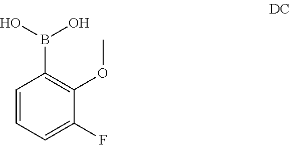

### 3-Iodo-4-methoxyquinoline (Intermediate DD)

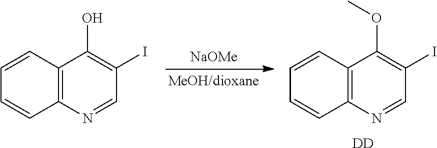

Sodium methoxide (5.4 M, 2.49 mL) was dissolved in dry MeOH (15 mL) then was added to a solution of 4-chloro-3-iodo-quinoline (3 g, 10.3 mmol, CAS #64965-48-6) in dry dioxane (30 mL) under N₂ atmosphere. Then the mixture was heated to 80° C. for 6 hrs. On completion, the reaction mixture was concentrated under reduced pressure to remove MeOH. The residue was diluted with H₂O (50 mL) and extracted with EA (50 mL×3). The combined organic layers were washed with brine (100 mL), dried over anhydrous sodium sulfate, filtered and concentrated under reduced pressure to give a residue. The residue was purified by column chromatography (SiO₂, Petroleum ether/Ethyl acetate=1/0 to 1/1) to give a title compound (2.3 g, 78% yield) as a white solid. ¹H NMR (400 MHz, DMSO-d₆) δ=9.08 (s, 1H), 8.13 (dd, J=0.8, 8.4 Hz, 1H), 8.08-8.03 (m, 1H), 7.83 (ddd, J=1.6, 6.8, 8.4 Hz, 1H), 7.71-7.66 (m, 1H), 4.02 (s, 3H).

### 1-(3-(7-Fluoro-2-(4-(3-methoxypyridin-2-yl)piperazine-1-carbonyl)-4-(4,4,5,5-tetramethyl-1,3,2-dioxaborolan-2-yl)-1H-indol-6-yl)-5,6-dihydropyridin-1(2H)-yl)-3-(1H-1,2,3-triazol-1-yl)propan-1-one (Intermediate DE)

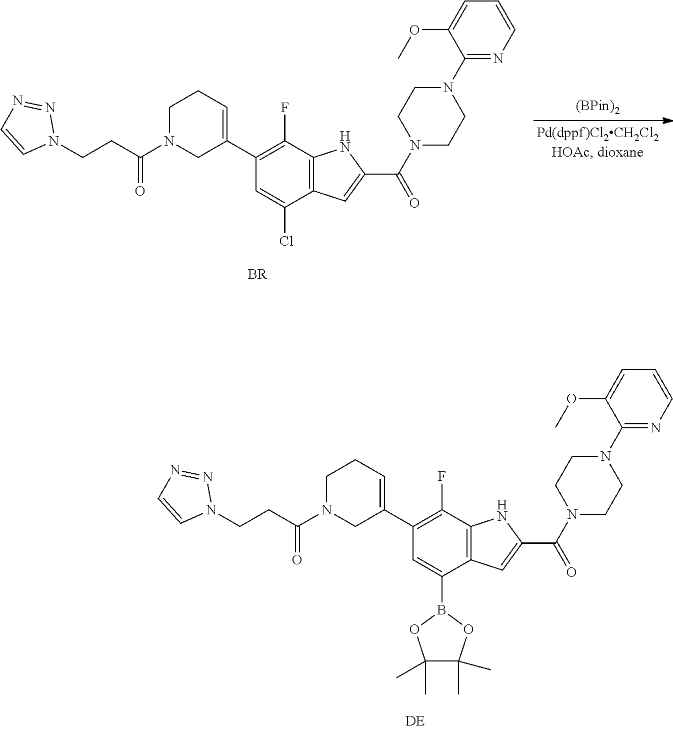 [SMILES: COc1cccnc1N1CCN(C(=O)c2cc3c(Cl)cc(C4=CCCN(C(=O)CCn5ccnn5)C4)c(F)c3[nH]2)CC1 ; COc1cccnc1N1CCN(C(=O)c2cc3c(B4OC(C)(C)C(C)(C)O4)cc(C4=CCCN(C(=O)CCn5ccnn5)C4)c(F)c3[nH]2)CC1]

A mixture of 1-[5-[4-chloro-7-fluoro-2-[4-(3-methoxy-2-pyridyl)piperazine-1-carbonyl]-1H-indol-6-yl]-3,6-dihydro-2H-pyridin-1-yl]-3-(triazol-1-yl)propan-1-one (300 mg, 505 μmol, Intermediate BR), 4,4,5,5-tetramethyl-2-(4,4,5,5-tetramethyl-1,3,2-dioxaborolan-2-yl)-1,3,2-dioxaborolane (385 mg, 1.52 mmol), KOAc (148 mg, 1.52 mmol), and XPhos Pd G3 (42.8 mg, 50.5 μmol) in dioxane (6 mL) was degassed and purged with N₂ three times. Then the mixture was stirred at 100° C. for 2 hrs under N₂ atmosphere. On completion, the reaction mixture was concentrated under reduced pressure to give a residue. The residue was purified by column chromatography (SiO₂, Petroleum ether/Ethyl acetate=1/1 to 0/1, DCM:MeOH=9:1) to give a title compound (390 mg, 77% yield) as a brown oil. LC-MS (ESI⁺) m/z 685.5 (M+H)⁺.

### (4-Methoxypyridin-3-yl)boronic acid (CAS #355004-67-0) (Intermediate DF)

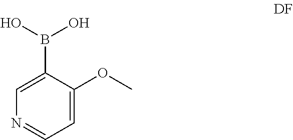

### 1-(5-(4-Chloro-7-fluoro-2-(4-phenylpiperazine-1-carbonyl)-1H-indol-6-yl)-3,6-dihydropyridin-1(2H)-yl)-3-(1H-1,2,3-triazol-1-yl)propan-1-one (Intermediate DG)

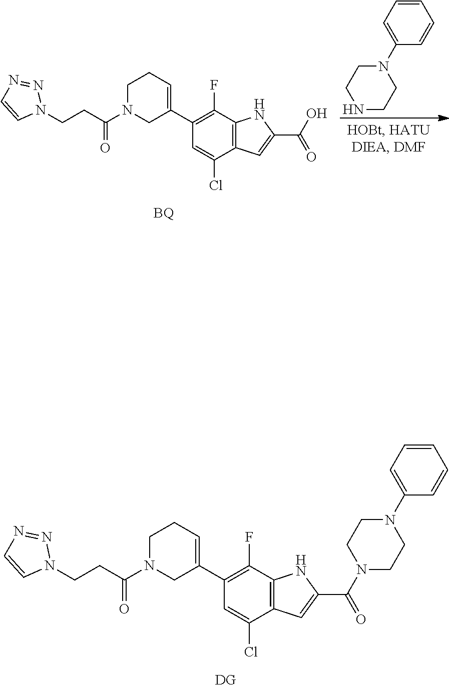 [SMILES: O=C(O)c1cc2c(Cl)cc(C3=CCCN(C(=O)CCn4ccnn4)C3)c(F)c2[nH]1 ; c1ccc(N2CCNCC2)cc1 ; O=C(CCn1ccnn1)N1CCC=C(c2cc(Cl)c3cc(C(=O)N4CCN(c5ccccc5)CC4)[nH]c3c2F)C1]

To a solution of 6-(1-(3-(1H-1,2,3-triazol-1-yl)propanoyl)-1,2,5,6-tetrahydropyridin-3-yl)-4-chloro-7-fluoro-1H-indole-2-carboxylic acid (300 mg, 718 μmol, Intermediate BQ) in DMF (4 mL) was added HOBt (116 mg, 861 μmol), HATU (354 mg, 933 μmol), DIEA (371 mg, 2.87 mmol, 500 μL) and 1-phenylpiperazine (116 mg, 718 μmol, 109 μL, CAS #92-54-6). Then the reaction was stirred at 25° C. for 10 min. On completion, the reaction mixture was diluted with water (10 mL) and extracted with ethyl acetate (3×10 mL). The combined organic layers were washed with brine (10 mL) and dried over anhydrous sodium sulfate, filtered and the filtrate was concentrated in vacuo to give the crude residue. The crude residue was purified by column chromatography (SiO₂, Petroleum ether/Ethyl acetate=10/1 to 0/1) to give the title compound (110 mg, 19% yield) as a yellow solid. LC-MS (ESI⁺) m/z 562.1 (M+H)⁺.

### Tert-butyl 3-bromo-4-isopropoxypyridine (Intermediate DH)

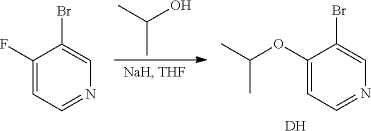

To a solution of propan-2-ol (341 mg, 5.68 mmol, CAS #67-63-0), NaH (227 mg, 5.68 mmol, 60% dispersion in mineral oil) in THF (4 mL) was dropwise added 3-bromo-4-fluoropyridine (500 mg, 2.84 mmol, CAS #116922-60-2) in THF (5 mL) at 0° C. under N₂ condition. The mixture was then stirred at 0° C. under N₂ for 2 hrs. On completion, the reaction mixture was quenched/diluted by sat. NH₄Cl (10 mL) and extracted by ethyl acetate (3×10 mL). The combined organic layers were washed with brine (10 mL) and dried over anhydrous sodium sulfate, filtered and the filtrate was concentrated in vacuo to give the crude residue. The residue was purified by column chromatography (SiO₂, Petroleum ether:Ethyl acetate=100/1 to 10/1) to give the title compound (380 mg, 62% yield) as a colorless oil. ¹H NMR (400 MHz, DMSO-d₆) δ=8.54 (s, 1H), 8.36 (d, J=5.6 Hz, 1H), 7.18 (d, J=5.6 Hz, 1H), 4.85 (t, J=6.0 Hz, 1H), 1.32 (d, J=6.0 Hz, 6H).

### 1-(3-(4-Chloro-7-fluoro-2-(4-(2-methoxyphenyl)piperazine-1-carbonyl)-1H-indol-6-yl)-5,6-dihydropyridin-1(2H)-yl)-3-(1H-1,2,3-triazol-1-yl)propan-1-one (Intermediate DI)

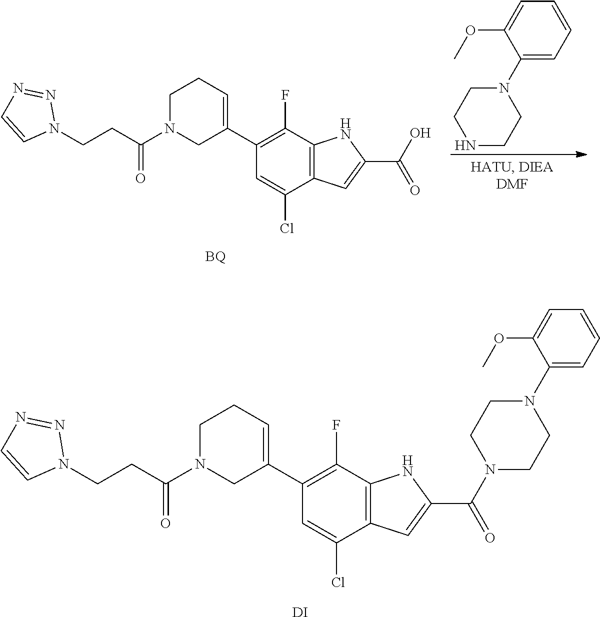

A solution of 4-chloro-7-fluoro-6-[1-[3-(triazol-1-yl)propanoyl]-3,6-dihydro-2H-pyridin-5-yl]-1H-indole-2-carboxylic acid (450 mg, 1.08 mmol, Intermediate BQ), 1-(2-methoxyphenyl)piperazine (248 mg, 1.29 mmol, CAS #35386-24-4), HATU (491 mg, 1.29 mmol), and DIEA (695 mg, 5.39 mmol, 938 μL) in DMF (5 mL) was stirred at 25° C. for 2 hrs. On completion, the reaction mixture was quenched with water (20 mL) and extracted with ethyl acetate (3×20 mL). The combined organic layers were washed with brine (20 mL) and dried over anhydrous sodium sulfate, filtered and the filtrate was concentrated in vacuo to give the crude residue. The crude residue was purified by reversed-phase HPLC (0.8 g/L ammonium bicarbonate) to give the title compound (330 mg, 42% yield) as a brown solid. LC-MS (ESI⁺) m/z 592.2 (M+H)⁺.

### 1-(3-(7-Fluoro-2-(4-(2-methoxyphenyl)piperazine-1-carbonyl)-4-(4,4,5,5-tetramethyl-1,3,2-dioxaborolan-2-yl)-1H-indol-6-yl)-5,6-dihydropyridin-1(2H)-yl)-3-(1H-1,2,3-triazol-1-yl)propan-1-one (Intermediate DJ)

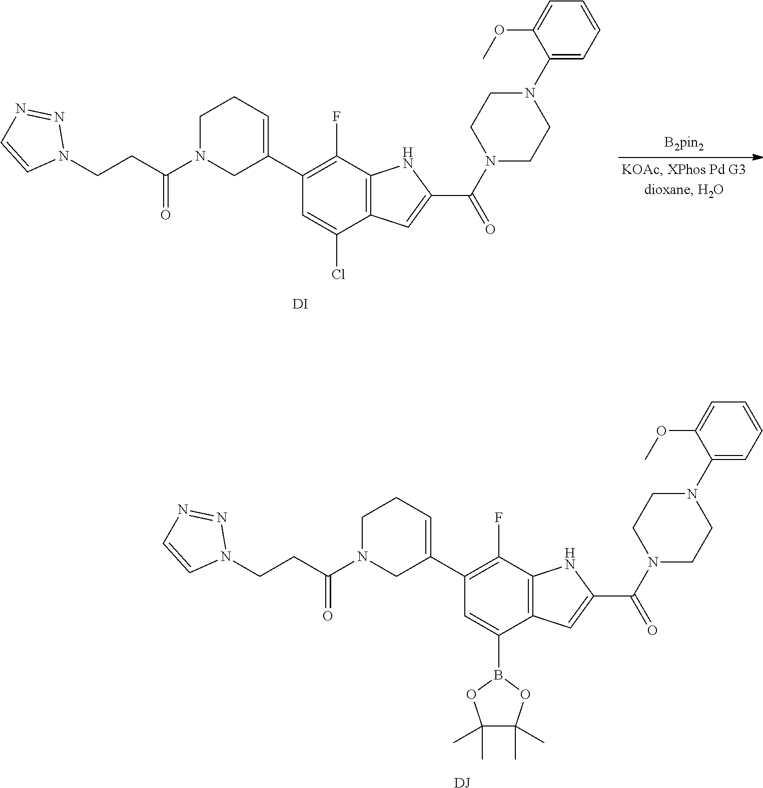

A solution of 1-(3-(4-chloro-7-fluoro-2-(4-(2-methoxyphenyl)piperazine-1-carbonyl)-1H-indol-6-yl)-5,6-dihydropyridin-1(2H)-yl)-3-(1H-1,2,3-triazol-1-yl)propan-1-one (330 mg, 557 μmol, Intermediate DI), 4,4,4′,4′,5,5,5′,5′-octamethyl-2,2′-bi(1,3,2-dioxaborolane) (283 mg, 1.11 mmol, CAS #73183-34-3), KOAc (164 mg, 1.67 mmol), and XPhos Pd G3 (47.1 mg, 55.7 μmol) in dioxane (6 mL) was stirred at 80° C. for 2 hrs. On completion, the reaction mixture was quenched with water (20 mL) and extracted with ethyl acetate (3×20 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=100/1 to 0/1) to give the tittle compound (306 mg, 68% yield) as a brown solid. LC-MS (ESI⁺) m/z 684.5 (M+H)⁺.

### 3-Bromo-4-(tert-butoxy)pyridine (Intermediate DK)

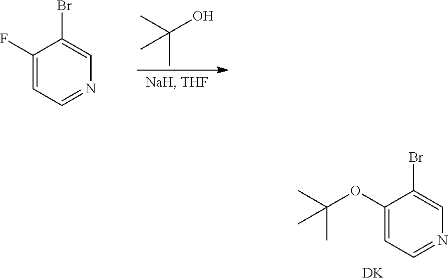

To a solution of 2-methylpropan-2-ol (842 mg, 11.3 mmol, CAS #75-65-0), NaH (454 mg, 11.3 mmol, 60% dispersion in mineral oil) in THF (5 mL) was dropwise added 3-bromo-4-fluoro-pyridine (1.00 g, 5.68 mmol, CAS #116922-60-2) in THF (10 mL) under N₂ condition. The mixture was then stirred at 0° C. under N₂ for 2 hrs. On completion, the reaction mixture was quenched with sat.NH₄Cl (10 mL) and extracted with ethyl acetate (3×10 mL). The combined organic layers were washed with brine (10 mL) and dried over anhydrous sodium sulfate, filtered and the filtrate was concentrated in vacuo to give the crude residue. The residue was purified by column chromatography (SiO₂, Petroleum ether:Ethyl acetate=100/1 to 10/1) to give the title compound (380 mg, 62% yield) as a colorless oil. ¹H NMR (400 MHz, DMSO-d₆) δ=8.59 (s, 1H), 8.33 (d, J=5.6 Hz, 1H), 7.30 (d, J=5.6 Hz, 1H), 1.49 (s, 9H).

### 3-Bromo-4-(fluoromethoxy)pyridine (Intermediate DL)

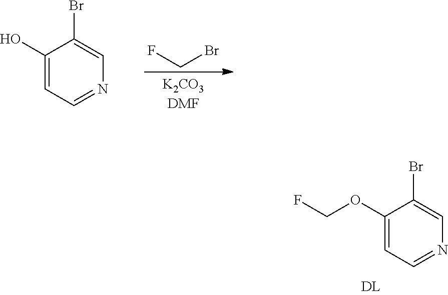

A solution of 3-bromopyridin-4-ol (1.00 g, 5.75 mmol, CAS #36953-41-0), bromofluoromethane (973 mg, 8.62 mmol, CAS #373-52-4), K₂CO₃ (2.38 g, 17.2 mmol) in DMF (10 mL) was stirred at 0° C. The mixture was stirred at 25° C. for 12 hrs. On completion, the reaction mixture was quenched/diluted by sat. NH₄Cl (10 mL) and extracted with ethyl acetate (3×10 mL). The combined organic layers were washed with brine (10 mL) and dried over anhydrous sodium sulfate, filtered and the filtrate was concentrated in vacuo to give the crude residue. The residue was purified by column chromatography (SiO₂, Petroleum ether:Ethyl acetate=100/1 to 10/1) to give the title compound (80 mg) as a white solid. ¹H NMR (400 MHz, DMSO-d₆) δ=8.70 (s, 1H), 8.50 (d, J=5.6 Hz, 1H), 7.35 (dd, J=1.2, 5.6 Hz, 1H), 6.14-6.00 (m, 2H).

### 3-Bromo-4-ethoxypyridine (Intermediate DM)

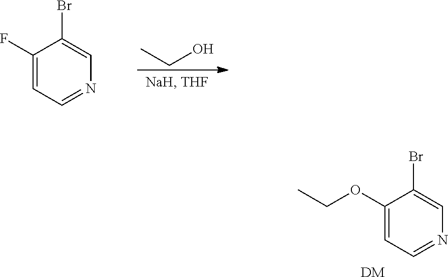

To a solution of ethanol (523 mg, 11.3 mmol, CAS #64-17-5), NaH (454 mg, 11.3 mmol, 60% dispersion in mineral oil) in THF (10 mL) was dropwise added 3-bromo-4-fluoropyridine (1.00 g, 5.68 mmol, CAS #116922-60-2) in THF (5 mL) at 0° C. The mixture was stirred at 25° C. for 12 hrs. On completion, the reaction mixture was quenched with sat.NH₄Cl (10 mL) and extracted with ethyl acetate (3×10 mL). The combined organic layers were washed with brine (10 mL) and dried over anhydrous sodium sulfate, filtered and the filtrate was concentrated in vacuo to give the crude residue. The residue was purified by column chromatography (SiO₂, Petroleum ether:Ethyl acetate=100/1 to 10/1) to give the title compound (80 mg) as a white solid. ¹H NMR (400 MHz, DMSO-d₆) δ=8.55 (s, 1H), 8.41-8.34 (m, 1H), 7.14 (d, J=5.6 Hz, 1H), 4.22 (q, J=6.8 Hz, 2H), 1.37 (t, J=7.2 Hz, 3H).

### 6-(1-Acetyl-1,2,5,6-tetrahydropyridin-3-yl)-7-fluoro-4-(3-methoxypyridin-4-yl)-1H-indole-2-carboxylic acid (Intermediate DN)

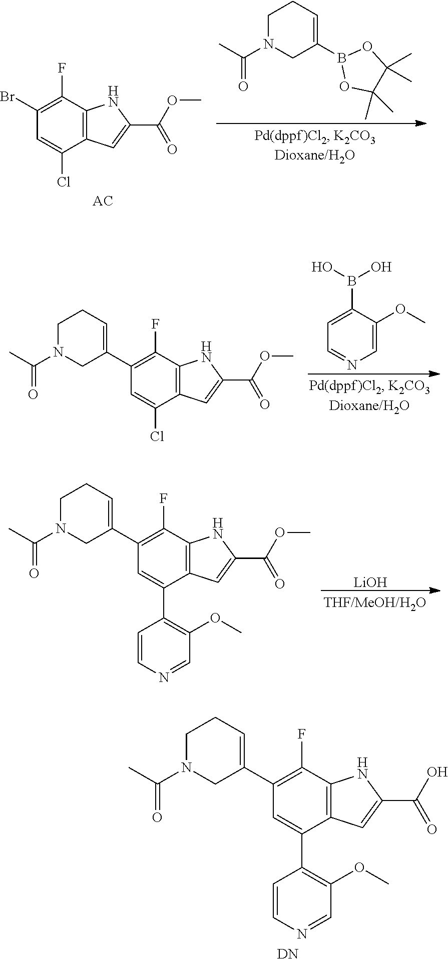

### Step 1—Methyl 6-(1-acetyl-1,2,5,6-tetrahydropyridin-3-yl)-4-chloro-7-fluoro-1H-indole-2-carboxylate

To a solution of methyl 6-bromo-4-chloro-7-fluoro-1H-indole-2-carboxylate (800 mg, 2.61 mmol, Intermediate AC), 1-(3-(4,4,5,5-tetramethyl-1,3,2-dioxaborolan-2-yl)-5,6-dihydropyridin-1(2H)-yl)ethanone (655 mg, 2.61 mmol, CAS #2101498-92-2) in dioxane (3 mL) and H₂O (1 mL) was added Pd(dppf)Cl₂ (190 mg, 261 μmol) and K₂CO₃ (1.08 g, 7.83 mmol) under nitrogen atmosphere. Then the reaction was stirred at 100° C. for 2 hrs under nitrogen atmosphere. On completion, the reaction mixture was diluted with water (15 mL) and extracted with ethyl acetate (3×15 mL). The combined organic layers were dried over anhydrous Na₂SO₄, filtered and the filtrate was concentrated in vacuo to give the crude residue. The residue was purified by column chromatography (SiO₂, DCM:Ethyl acetate=100/1 to 0/1) to give the title compound (800 mg, 87% yield) as a yellow solid. LC-MS (ESI⁺) m/z 351.1 (M+H)⁺.

### Step 2—Methyl 6-(1-acetyl-1,2,5,6-tetrahydropyridin-3-yl)-7-fluoro-4-(3-methoxypyridin-4-yl)-1H-indole-2-carboxylate

To a solution of methyl 6-(1-acetyl-1,2,5,6-tetrahydropyridin-3-yl)-4-chloro-7-fluoro-1H-indole-2-carboxylate (300 mg, 855 μmol), (3-methoxypyridin-4-yl)boronic acid (156 mg, 1.03 mmol, CAS #1008506-24-8) in dioxane (3 mL) and H₂O (1 mL) was added and Pd(dppf)Cl₂ (62.5 mg, 85.5 μmol) and K₂CO₃ (354 mg, 2.57 mmol) under nitrogen atmosphere. Then the reaction was stirred at 80° C. for 2 hrs under nitrogen atmosphere. On completion, the reaction mixture was concentrated in vacuo to give the crude residue. The residue was purified by column chromatography (SiO₂, DCM:Ethyl acetate=0/1 to 100/1) to give the title compound (200 mg, 55% yield) as a yellow solid. LC-MS (ESI⁺) m/z 424.4 (M+H)⁺.

### Step 3—6-(1-Acetyl-1,2,5,6-tetrahydropyridin-3-yl)-7-fluoro-4-(3-methoxypyridin-4-yl)-1H-indole-2-carboxylic acid

To a solution of methyl 6-(1-acetyl-1,2,5,6-tetrahydropyridin-3-yl)-7-fluoro-4-(3-methoxypyridin-4-yl)-1H-indole-2-carboxylate (0.2 g, 472 μmol) in THF (1 mL), MeOH (1 mL), H₂O (1 mL) was added LiOH (198 mg, 4.72 mmol). The mixture was then stirred at 40° C. for 2 hrs. On completion, the reaction mixture was added HCl (1N) until the pH was 1, then diluted with water (3 mL) and extracted with ethyl acetate (3×5 mL). The combined organic layers were dried over anhydrous sodium sulfate, filtered and the filtrate was concentrated in vacuo to give the title compound (100 mg) as a yellow solid. LC-MS (ESI⁺) m/z 410.2 (M+H)⁺.

### 6-(1-Acetyl-1,2,5,6-tetrahydropyridin-3-yl)-4-chloro-7-fluoro-1H-indole-2-carboxylic acid (Intermediate DO)

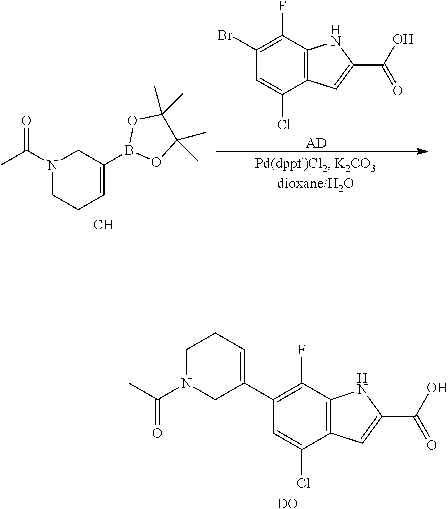

A mixture of 1-(5-(4,4,5,5-tetramethyl-1,3,2-dioxaborolan-2-yl)-3,6-dihydropyridin-1(2H)-yl)ethan-1-one (1.55 g, 6.15 mmol, Intermediate CH), 6-bromo-4-chloro-7-fluoro-1H-indole-2-carboxylic acid (2 g, 6.84 mmol, Intermediate AD), Pd(dppf)Cl₂ (500 mg, 684 μmol), and K₂CO₃ (2.84 g, 20.5 mmol) in dioxane (3 mL) and H₂O (0.3 mL) was degassed and purged with N₂ three times. Then the mixture was stirred at 80° C. for 1 hr under N₂ atmosphere. On completion, the reaction mixture was quenched with water (30 mL), then HCl (1N) was added until pH 7. Then the reaction mixture was filtered and filter cake was concentrated in vacuo to give the title compound (1.9 g) as a white solid. LC-MS (ESI⁺) m/z 336.9 (M+H)⁺.

### 1-(3-(4-Chloro-7-fluoro-2-(4-(2-methoxyphenyl)piperazine-1-carbonyl)-1H-indol-6-yl)-5,6-dihydropyridin-1(2H)-yl)ethanone (Intermediate DP)

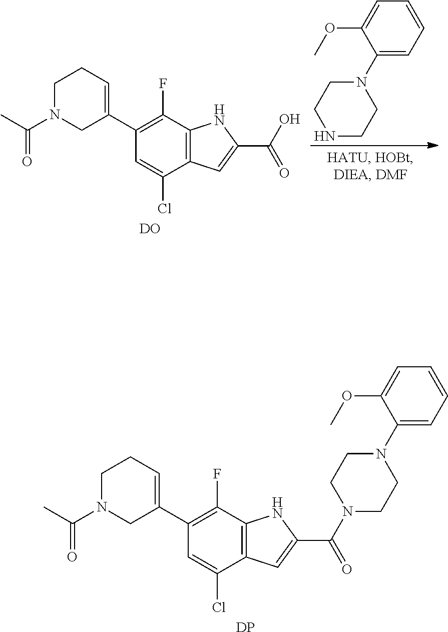

To a solution of 6-(1-acetyl-1,2,5,6-tetrahydropyridin-3-yl)-4-chloro-7-fluoro-1H-indole-2-carboxylic acid (200 mg, 594 μmol, Intermediate DO) in DMF (6 mL) was added HATU (294 mg, 772 μmol), DIEA (384 mg, 2.97 mmol, 517.24 μL), HOBt (161 mg, 1.19 mmol) and 1-(2-methoxyphenyl)piperazine (114 mg, 593 μmol, 103 μL, CAS #35386-24-4). The mixture was then stirred at 25° C. for 10 min. On completion, the reaction mixture was quenched with water (20 mL) and solid formed, filtered and the filter cake was dried in vacuo to give the title compound (250 mg) as yellow solid. LC-MS (ESI⁺) m/z 511.6 (M+H)⁺.

### (2-Methoxypyridin-3-yl)boronic acid (CAS #163105-90-6) (Intermediate DO)

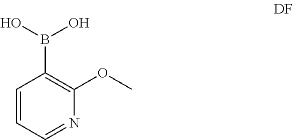

### (3-Chloro-2-methoxyphenyl)boronic acid (CAS #179898-50-1) (Intermediate DR)

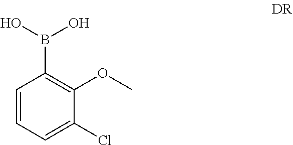

### (2-Ethoxyphenyl)boronic acid (CAS #213211-69-9) (Intermediate DS)

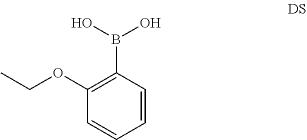

### 5-Bromo-2-formyl-1,2,3,4-tetrahydroisoquinoline-3-carboxylic acid (Intermediate DT)

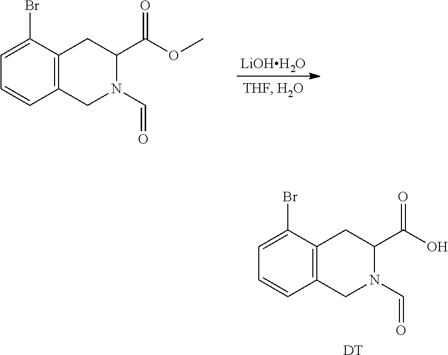

To a solution of methyl 5-bromo-2-formyl-1,2,3,4-tetrahydroisoquinoline-3-carboxylate (300 mg, 1.01 mmol, synthesized via Steps 1-6 of Intermediate BT) in tetrahydrofuran (1.5 mL) and water (1.5 mL) was added lithium hydroxide monohydrate (84 mg, 2.01 mmol). The mixture was then stirred at 25° C. for 1 h. On completion, the mixture was adjusted pH to 4-5 with 1 M hydrochloric acid aqueous solution. The mixture was diluted with water (10 mL) and extracted with ethyl acetate (10 mL×4). The combined organic layers were washed with brine (10 mL) and dried with sodium sulfate solid and filtered. Then the filtrate was concentrated under reduced pressure to dryness to give the title compound (180 mg, 62% yield) as a white solid. LC-MS (ESI⁺) m/z 285.8 (M+H)⁺.

### 5-(1-(5-Bromo-2-formyl-1,2,3,4-tetrahydroisoquinoline-3-carbonyl)-1,2,5,6-tetrahydropyridin-3-yl)-N,N-dimethylbenzofuran-2-carboxamide (Intermediate DU)

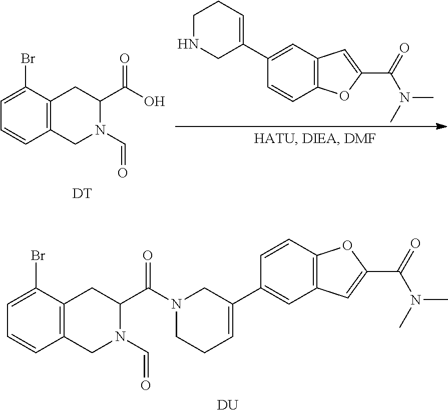 [SMILES: O=CN1Cc2cccc(Br)c2CC1C(=O)O ; CN(C)C(=O)c1cc2cc(C3=CCCNC3)ccc2o1 ; "DT" ; CN(C)C(=O)c1cc2cc(C3=CCCN(C(=O)C4Cc5c(Br)cccc5CN4C=O)C3)ccc2o1 ; "DU"]

To a solution of 5-bromo-2-formyl-1,2,3,4-tetrahydroisoquinoline-3-carboxylic acid (170 mg, 598 umol, Intermediate DT) in dimethylformamide (2.0 mL) was added O-(7-azabenzotriazol-1-yl)-N,N,N,N-tetramethyluroniumhexafluorophosphate (341 mg, 898 umol) and NN-diisopropylethylamine (386 mg, 2.99 mmol, 521 uL). The mixture was stirred at 25° C. for 10 min. Next, N,N-dimethyl-5-(1,2,5,6-tetrahydropyridin-3-yl)benzofuran-2-carboxamide (194 mg, 598 umol, 1.5 hydrochloric acid salt, Intermediate BU) was added to the solution and the mixture was stirred at 25° C. for 50 min. On completion, the mixture was diluted with water (20 mL) and extracted with ethyl acetate (20 mL×2). The combined organic layers were washed with brine (20 mL) and dried with sodium sulfate solid and filtered. Then the filtrate was concentrated under reduced pressure to dryness. The residue was purified by flash silica gel chromatography (ISCO®; 12 g Sepa Flash® Silica Flash Column, Eluent of 0˜100% Ethyl acetate/Petroleum ether gradient @40 mL/min) to give the title compound (183 mg, 55% yield) as a white solid. LC-MS (ESI⁺) m/z 536.1 (M+H)⁺.

### 1-Bromo-2-methoxy-benzene (CAS #578-57-4) (Intermediate DV)

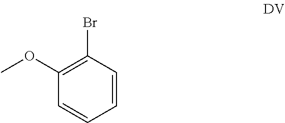

### 2-(2-Methoxyphenyl)-4,4,5,5-tetramethyl-1,3,2-dioxaborolane (CAS #190788-60-4) (Intermediate DW)

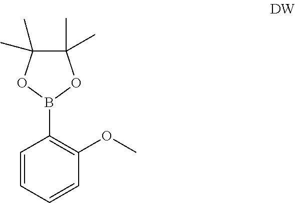

### 1-(5-(4-Chloro-7-fluoro-2-(4-(3-methoxypyridin-2-yl)piperazine-1-carbonyl)-1H-indol-6-yl)-3,6-dihydropyridin-1(2H)-yl)ethan-1-one (Intermediate DX)

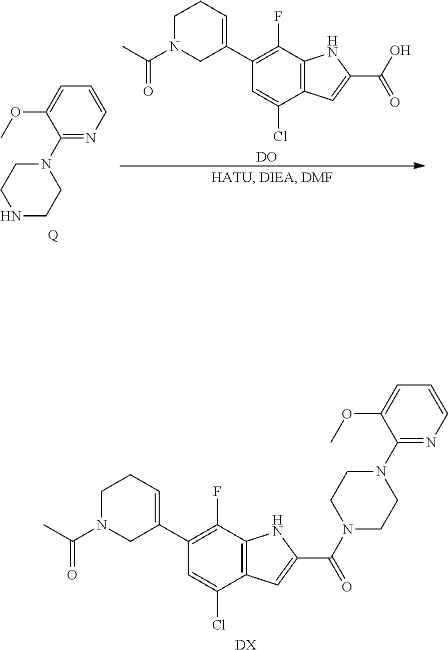

To a solution of 6-(1-acetyl-1,2,5,6-tetrahydropyridin-3-yl)-4-chloro-7-fluoro-1H-indole-2-carboxylic acid (174 mg, 5178 μmol, Intermediate DO) in DMF (5 mL) was added HATU (236 mg, 620 μmol) and DIEA (334 mg, 2.59 mmol, 450 μL), and 1-(3-methoxypyridin-2-yl)piperazine (100 mg, 517 μmol, Intermediate Q). Then the mixture was stirred at 25° C. for 1 hr. On completion, the mixture was quenched with water (20 mL) and filtered, then the filter cake was dried to give the title compound (240 mg) as brown solid. LC-MS (ESI⁺) m/z 512.1 (M+H)⁺.

### 1-(3-Cyclopropoxy-5-cyclopropylpyridin-2-yl)piperazine (Intermediate DY)

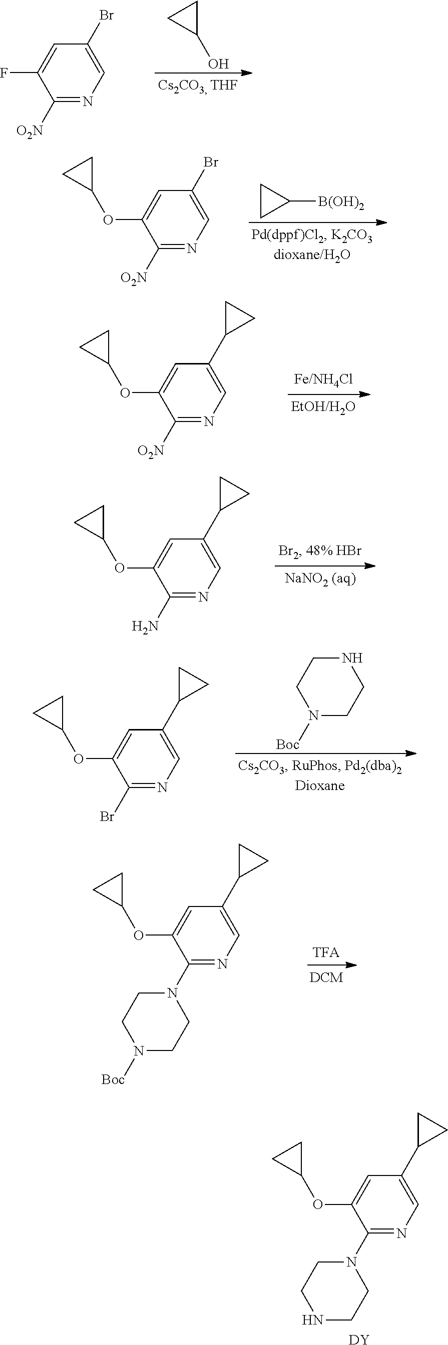

### Step 1—5-Bromo-3-cyclopropoxypyridin-2-amine

To a solution of 5-bromo-3-fluoro-2-nitro-pyridine (14 g, 63 mmol, CAS #1532517-95-5) in anhydrous THF (120 mL) was added cyclopropanol (9.20 g, 158 mmol) and Cs₂CO₃ (51.6 g, 158 mmol) under nitrogen atmosphere, then the reaction was stirred at 65° C. for 12 h under nitrogen atmosphere. On completion, the mixture was filtered and the filtrate was concentrated to give a crude residue. The residue was purified by column chromatography (SiO₂, Petroleum ether:Ethyl acetate=10/1 to 4/1) to give the title compound (14 g, 85% yield) as a yellow solid. LC-MS (ESI⁺) m/z 258.8 (M+H)⁺.

### Step 2—3-Cyclopropoxy-5-cyclopropyl-2-nitropyridine

A mixture of 5-bromo-3-cyclopropoxy-2-nitropyridine (4 g, 15 mmol), cyclopropylboronic acid (1.86 g, 21.6 mmol, CAS #411235-57-9), Pd(dppf)Cl₂ CH₂Cl₂ (1.89 g, 2.32 mmol), and K₂CO₃ (6.40 g, 46.3 mmol) in dioxane (40 mL), H₂O (10 mL) was degassed and purged with N₂ three times. Then the mixture was stirred at 80° C. for 2 hrs under N₂ atmosphere. On completion, the reaction mixture was concentrated under reduced pressure to give a residue. The residue was purified by column chromatography (SiO₂, Petroleum ether/Ethyl acetate=20/1 to 8/1) to give the title compound (2.5 g, 70% yield) as a brown oil. LC-MS (ESI⁺) m/z 221.0 (M+H)⁺.

### Step 3—3-Cyclopropoxy-5-cyclopropylpyridin-2-amine

To a solution of 3-cyclopropoxy-5-cyclopropyl-2-nitropyridine (2.2 g, 10 mmol) in EtOH (10 mL), H₂O (10 mL) was added Fe (2.79 g, 49.9 mmol) and NH₄Cl (4.27 g, 79.9 mmol). The mixture was then stirred at 70° C. for 12 hrs. On completion, the reaction mixture was filtered and the filtrate was concentrated in vacuo to give the crude residue. The residue was purified by column chromatography (SiO₂, Petroleum ether:Ethyl acetate=10/1 to 4/1) to give the title compound (1.9 g, 89% yield) as a brown solid. LC-MS (ESI⁺) m/z 191.1 (M+H)⁺.

### Step 4—2-Bromo-3-cyclopropoxy-5-cyclopropylpyridine

A solution of 3-cyclopropoxy-5-cyclopropylpyridin-2-amine (800 mg, 4.21 mmol) was slowly added to HBr (19.0 g, 117 mmol, 12.8 mL, 50% solution) under N₂ atmosphere. The mixture was cooled to 0° C. Then Br₂ (2.02 g, 12.6 mmol) was added, followed by NaNO₂ (725 mg, 10.5 mmol) in H₂O (4 mL). The resulting mixture was stirred at 0° C. for 1 hr under N₂ atmosphere. On completion, the reaction mixture was quenched by addition of 30% NaOH (10 mL) at 0° C. The crude residue was purified by reversed-phase HPLC (0.1% FA condition) to give the title compound (700 mg, 65% yield) as a white solid. LC-MS (ESI⁺) m/z 256.0 (M+H)⁺.

### Step 5—Tert-butyl 4-(3-cyclopropoxy-5-cyclopropylpyridin-2-yl)piperazine-1-carboxylate

A mixture of 2-bromo-3-cyclopropoxy-5-cyclopropylpyridine (700 mg, 2.75 mmol), tert-butyl piperazine-1-carboxylate (820 mg, 4.41 mmol, CAS #57260-71-6), RuPhos (128 mg, 275 μmol), Cs₂CO₃ (2.69 g, 8.26 mmol) and Pd₂(dba)₃ (126 mg, 137 μmol) in dioxane (7 mL) was degassed and purged with N₂ three times. Then the mixture was stirred at 100° C. for 12 hrs under N₂ atmosphere. On completion, the reaction mixture was concentrated under reduced pressure to give a residue. The residue was purified by column chromatography (SiO₂, Petroleum ether/Ethyl acetate=8/1 to 4/1) to give the title compound (150 mg, 15% yield) as a brown solid. LC-MS (ESI⁺) m/z 360.6 (M+H)⁺.

### Step 6—1-(3-Cyclopropoxy-5-cyclopropylpyridin-2-yl)piperazine

A mixture of tert-butyl 4-(3-cyclopropoxy-5-cyclopropylpyridin-2-yl)piperazine-1-carboxylate (120 mg, 333 μmol) in DCM (2 mL) was added TFA (921 mg, 8.08 mmol). The mixture was stirred at 25° C. for 1 hr under N₂ atmosphere. On completion, the reaction mixture was concentrated in vacuo to give the title compound (120 mg) as a brown oil. LC-MS (ESI⁺) m/z 260.1 (M+H)⁺.

### (3-Methoxy-4-pyridyl)boronic acid (CAS #1008506-24-8) (Intermediate DZ)

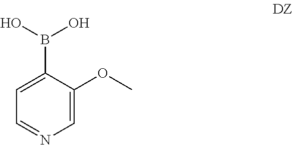

### 6-(1-Acetyl-1,2,5,6-tetrahydropyridin-3-yl)-4-chloro-7-fluoro-N,N-dimethylbenzo[b]thiophene-2-carboxamide (Intermediate EA)

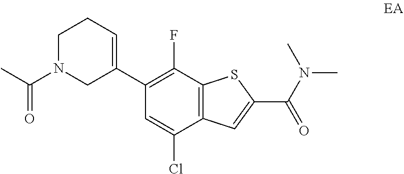 [SMILES: CC(=O)N1CCC=C(c2cc(Cl)c3cc(C(=O)N(C)C)sc3c2F)C1]

6-(1-Acetyl-1,2,5,6-tetrahydropyridin-3-yl)-4-chloro-7-fluoro-N,N-dimethylbenzo[b]thiophene-2-carboxamide was synthesized via Steps 1-7 of Intermediate K. ¹H NMR (400 MHz, DMSO-d₆) δ=7.81-7.74 (m, 1H), 7.64-7.50 (m, 1H), 6.32-6.22 (m, 1H), 4.31 (s, 2H), 3.64-3.56 (m, 2H), 3.31-3.20 (m, 3H), 3.05 (s, 3H), 2.40-2.35 (m, 1H), 2.30-2.23 (m, 1H), 2.07 (d, J=6.0 Hz, 3H).

### 1-(2-Methoxy-6-phenoxyphenyl)piperazine (Intermediate EB)

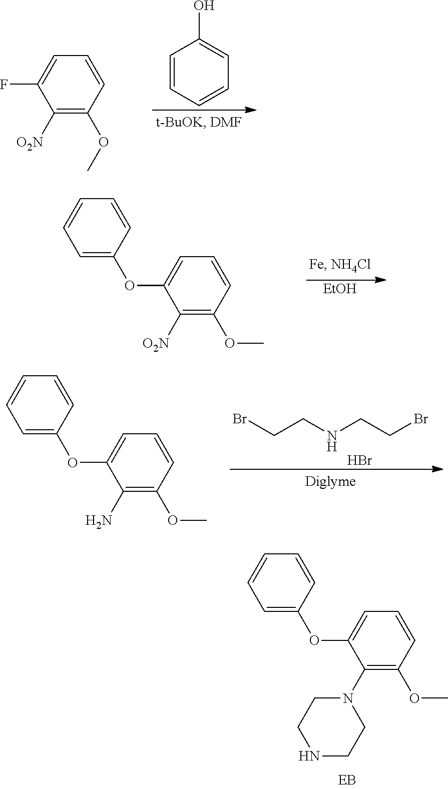

### Step 1—1-Methoxy-2-nitro-3-phenoxybenzene

To a solution of 1-fluoro-3-methoxy-2-nitro-benzene (2 g, 12 mmol) in DMF (20 mL) was added t-BuOK (2.62 g, 23.3 mmol) and phenol (1.32 g, 14.0 mmol, 1.23 mL). The mixture was then stirred at 120° C. for 12 h. On completion, the reaction mixture was diluted with H₂O (20 mL) and extracted with EA (20 mL). The combined organic layers were washed with H₂O (20 mL), dried over NA₂SO₄, filtered and concentrated under reduced pressure to give a residue. The crude product was purified by reversed-phase HPLC (0.1% FA condition) to give the title compound (1 g, 35% yield) as a yellow oil. LC-MS (ESI⁺) m/z 246.0 (M+H)⁺.

### Step 2—2-Methoxy-6-phenoxyaniline

To a solution of 1-methoxy-2-nitro-3-phenoxy-benzene (1 g, 4 mmol) in H₂O (10 mL), EtOH (10 mL) was added Fe (1.82 g, 32.6 mmol) and NH₄Cl (1.75 g, 32.6 mmol). The mixture was then stirred at 80° C. for 12 h. On completion, the reaction mixture was diluted with H₂O (10 mL) and extracted with EA (10 mL×3). The combined organic layers were washed with H₂O (20 mL), dried over Na₂SO₄, filtered and concentrated under reduced pressure to give a residue. The residue was purified by column chromatography (SiO₂, Petroleum ether/Ethyl acetate=10/1 to 8/1) to give the title compound (600 mg, 68% yield) as a yellow oil. LC-MS (ESI⁺) m/z 216.0 (M+H)⁺.

### Step 3—1-(2-Methoxy-6-phenoxyphenyl)piperazine

To a solution of 2-methoxy-6-phenoxy-aniline (400 mg, 1.86 mmol) in 1-methoxy-2-(2-methoxyethoxy)ethane (0.5 mL) was added 2-bromo-N-(2-bromoethyl)ethanamine (643.71 mg, 2.79 mmol). The mixture was then stirred at 130° C. for 6 h. On completion, the reaction mixture was added NaHCO₃ (aq) until pH ˜9, then filtered and the filtrate was concentrated in vacuo to give the crude residue. The crude product was purified by reversed-phase HPLC (0.1% FA condition) to give the title compound (60 mg, 11% yield, FA) as a black oil. LC-MS (ESI⁺) m/z 285.0 (M+H)⁺.

### 1-(3-Methoxy-4-methylpyridin-2-yl)piperazine (Intermediate EC)

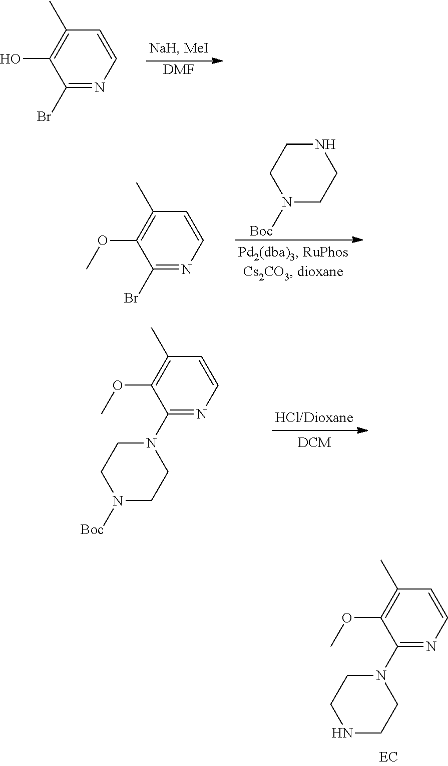

### Step 1—2-Bromo-3-methoxy-4-methylpyridine

To a solution of 2-bromo-4-methylpyridin-3-ol (800 mg, 4.25 mmol, CAS #1227578-74-6) in DMF (8 mL) was added NaH (255 mg, 6.38 mmol, 60% dispersion in mineral oil) under N₂ atmosphere at 0° C. The mixture was stirred at 0° C. for 0.5 hours, and then was added MeI (905 mg, 6.38 mmol, 397 μL) at 0° C. The resulting mixture was then stirred at 25° C. for 12 hrs. On completion, the reaction mixture was quenched with sat. NH₄Cl (20 mL) and extracted with ethyl acetate (3×50 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 10/1) to give the title compound (700 mg, 80% yield) as a yellow oil. ¹H NMR (400 MHz, CHLOROFORM-d) δ=8.00 (d, J=4.8 Hz, 1H), 7.07 (d, J=4.8 Hz, 1H), 3.85 (s, 3H), 2.35 (s, 3H).

### Step 2—Tert-butyl 4-(3-methoxy-4-methylpyridin-2-yl)piperazine-1-carboxylate

To a solution of 2-bromo-3-methoxy-4-methylpyridine (600 mg, 2.97 mmol), tert-butyl piperazine-1-carboxylate (829 mg, 4.45 mmol, CAS #57260-71-6) in dioxane (8 mL) was added Cs₂CO₃ (2.90 g, 8.91 mmol), RuPhos (138 mg, 296 μmol) and Pd₂(dba)₃ (271 mg, 296 μmol). Then the reaction was stirred at 80° C. for 6 hrs under nitrogen atmosphere. On completion, the reaction mixture was filtered and the filtrate was concentrated in vacuo to give the crude residue. The crude residue was purified by column chromatography (SiO₂, Petroleum ether/Ethyl acetate=1/0 to 10/1) to give the title compound (600 mg, 65% yield) as a yellow solid. LC-MS (ESI⁺) m/z 308.3 (M+H)⁺.

### Step 3—1-(3-Methoxy-4-methylpyridin-2-yl)piperazine

To a solution of tert-butyl 4-(3-methoxy-4-methylpyridin-2-yl)piperazine-1-carboxylate (600 mg, 1.95 mmol) in DCM (6 mL) was added HCl/dioxane (8 M, 2 mL). The mixture was then stirred at 25° C. for 1 hr. On completion, the reaction mixture was filtered and the filter cake was concentrated in vacuo to give the title compound (400 mg, HCl) as a white solid. LC-MS (ESI⁺) m/z 208.2 (M+H)⁺.

### 5-Methoxy-N,N-dimethyl-6-(piperazin-1-yl)pyridin-3-amine (Intermediate ED)

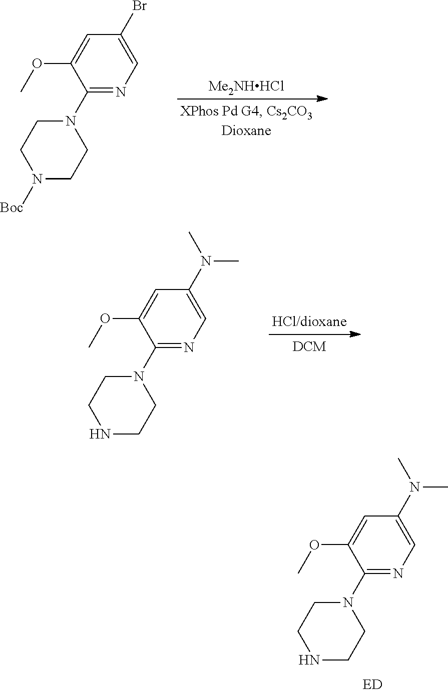

### Step 1—5-Methoxy-N,N-dimethyl-6-(piperazin-1-yl)pyridin-3-amine

A mixture of tert-butyl 4-(5-bromo-3-methoxypyridin-2-yl)piperazine-1-carboxylate (1 g, 2.69 mmol, Step 1 of Intermediate AY), N-methylmethanamine hydrochloride (1.10 g, 13.4 mmol), Xphos Pd G4 (346 mg, 402 μmol), Cs₂CO₃ (2.63 g, 8.06 mmol) and TEA (1.63 g, 16.1 mmol) in dioxane (15 mL) was degassed and purged with N₂ three times. Then the mixture was stirred at 100° C. for 4 hrs under N₂ atmosphere. On completion, the reaction mixture was concentrated under reduced pressure to give a residue. The crude residue was purified by reversed-phase HPLC (0.1% FA condition) to give the title compound (200 mg, 23% yield) as a brown solid. LC-MS (ESI⁺) m/z 337.1 (M+H)⁺.

### Step 2—5-Methoxy-N,N-dimethyl-6-(piperazin-1-yl)pyridin-3-amine

To a solution of tert-butyl 4-[5-(dimethylamino)-3-methoxy-2-pyridyl]piperazine-1-carboxylate (200 mg, 594 μmol) in DCM (2 mL) was added HCl/dioxane (4 M, 148 μL). The mixture was then stirred at 25° C. for 1 hr. On completion, the reaction mixture was concentrated in vacuo to give the title compound (200 mg) as a brown solid. LC-MS (ESI⁺) m/z 238.1 (M+H)⁺.

### 5-(3-Methoxypyridin-2-yl)-1,2,3,5-tetrahydropyrrolo[3,4-c]pyrrole (Intermediate EE)

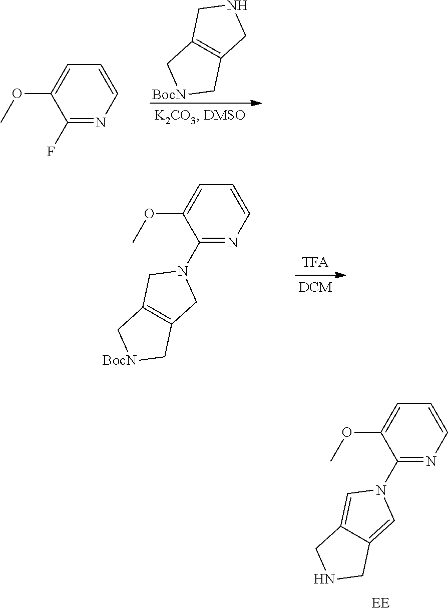

### Step 1—Tert-butyl 5-(3-methoxypyridin-2-yl)-3,4,5,6-tetrahydropyrrolo[3,4-c]pyrrole-2(1H)-carboxylate

To a solution of 2-fluoro-3-methoxypyridine (302 mg, 2.38 mmol, CAS #163234-74-0), tert-butyl 3,4,5,6-tetrahydropyrrolo[3,4-c]pyrrole-2(1H)-carboxylate (0.5 g, 2.38 mmol, CAS #169692-94-8) in DMSO (5 mL) was added K₂CO₃ (985 mg, 7.13 mmol). The mixture was then stirred at 120° C. for 12 hrs. On completion, to the reaction mixture was added water (15 mL) and the mixture was extracted with ethyl acetate (3×50 mL). The combined organic layers were dried over anhydrous sodium sulfate, filtered and the filtrate was concentrated in vacuo to give the crude residue. The crude residue was purified by reversed-phase HPLC (0.8 g/L ammonium bicarbonate) to give the title compound (100 mg, 13% yield) as a brown solid. LC-MS (ESI⁺) m/z 318.0 (M+H)⁺.

### Step 2—5-(3-Methoxypyridin-2-yl)-1,2,3,5-tetrahydropyrrolo[3,4-c]pyrrole

To a solution of tert-butyl 5-(3-methoxypyridin-2-yl)-3,4,5,6-tetrahydropyrrolo[3,4-c]pyrrole-2(1H)-carboxylate (100 mg, 315 μmol) in DCM (2 mL) was added TFA (767 mg, 6.73 mmol). The mixture was then stirred at 25° C. for 2 hrs. On completion, the reaction mixture was concentrated in vacuo to give the title compound (150 mg) as a brown oil. LC-MS (ESI⁺) m/z 215.9 (M+H)⁺.

### 1-(2-Fluorophenyl)piperazine (Intermediate EF)

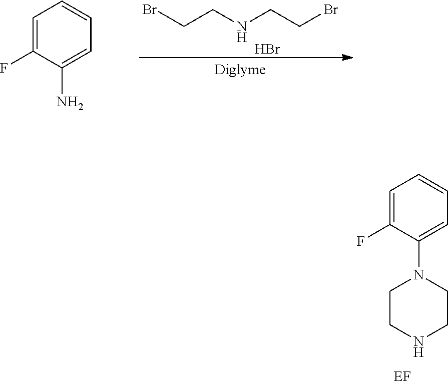 [SMILES: Nc1ccccc1F ; BrCCNCCBr ; Br ; Fc1ccccc1N1CCNCC1]

To a solution of 2-fluoroaniline (1 g, 9.00 mmol, 867 L, CAS #348-54-9) and 2-bromo-N-(2-bromoethyl)ethanamine (3.12 g, 13.5 mmol, CAS #3890-99-1) in 2-(2-methoxyethoxy)ethanol (2 mL) was stirred at 130° C. for 4 hrs. On completion, the reaction mixture was triturated with acetone at 0° C. for 15 min to give the title compound (600 mg) as a white solid. ¹H NMR (400 MHz, DMSO-d₆) δ=8.83 (br s, 1H), 7.22-7.02 (m, 4H), 4.56 (br s, 4H), 3.26 (br s, 4H).

### 1-Phenylpiperazine (CAS #92-54-6) (Intermediate EG)

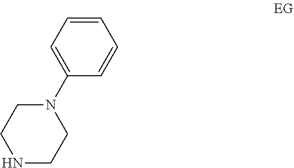 [SMILES: c1ccc(N2CCNCC2)cc1]

### 1-(2-(Trifluoromethoxy)phenyl)piperazine (Intermediate EH)

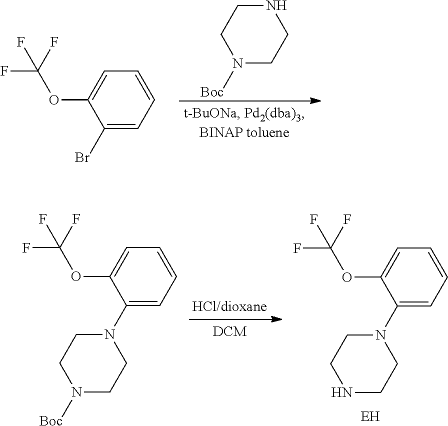 [SMILES: FC(F)(F)Oc1ccccc1Br ; CC(C)(C)OC(=O)N1CCNCC1 ; CC(C)(C)OC(=O)N1CCN(c2ccccc2OC(F)(F)F)CC1 ; FC(F)(F)Oc1ccccc1N1CCNCC1]

### Step 1—Tert-butyl 4-(2-(trifluoromethoxy)phenyl)piperazine-1-carboxylate

A solution of 1-bromo-2-(trifluoromethoxy)benzene (1.07 g, 4.44 mmol, CAS #64115-88-4), tert-butyl piperazine-1-carboxylate (910 mg, 4.89 mmol, CAS #57260-71-6), Pd₂(dba)₃ (406 mg, 444 mol), t-BuONa (854 mg, 8.89 mmol), and BINAP (553 mg, 888 μmol) in toluene (10 mL) was stirred at 100° C. for 2 hrs under N₂. On completion, the reaction mixture was filtered and the filtrate was concentrated in vacuo to give the crude residue. The residue was purified by column chromatography (SiO₂, Petroleum ether:Ethyl acetate=100/1 to 10/1) to give the title compound (1.00 g, 58% yield) as a yellow oil. LC-MS (ESI⁺) m/z 541.3 (M+H)⁺.

### Step 2—1-(2-(Trifluoromethoxy)phenyl)piperazine

A solution of tert-butyl 4-(2-(trifluoromethoxy) phenyl) piperazine-1-carboxylate (1.00 g, 2.89 mmol) in DCM (10 mL) and HCl/dioxane (3 mL) was stirred at 25° C. for 2 hrs. On completion, the crude residue was concentrated in vacuo to give the tittle compound (600 mg) as a yellow solid. LC-MS (ESI⁺) m/z 246.9 (M+H)⁺.

### 1-(O-tolyl)piperazine (Intermediate EI)

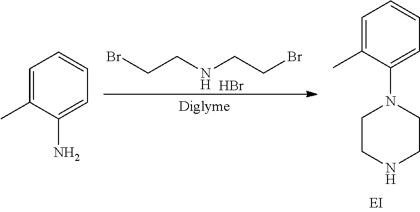

To a solution of o-toluidine (1 g, 9.00 mmol, 867 μL, CAS #95-53-4) and 2-bromo-N-(2-bromoethyl)ethanamine (3.12 g, 13.5 mmol, CAS #3890-99-1) in 2-(2-methoxyethoxy)ethanol (2 mL) was stirred at 130° C. for 4 hrs. On completion, the reaction mixture was triturated with acetone at 0° C. for 15 min to give the title compound (600 mg) as a white solid.

### 4-(2-methoxyphenyl)piperidine CAS #5833-75-8) (Intermediate EJ)

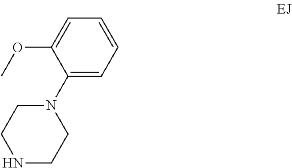

### 1-(2-chlorophenyl)piperazine (CAS #39512-50-0) (Intermediate EK)

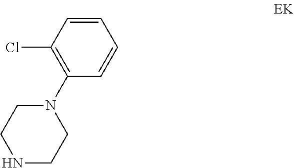 [SMILES: Clc1ccccc1N1CCNCC1]

### 1-(2,3,4-trifluorophenyl)piperazine (Intermediate EL), 9-methyl-2-(piperazin-1-yl)-9H-carbazole (Intermediate EM), 4-(piperazin-1-yl)-1H-indole-6-carbonitrile (Intermediate EN), 1-(pyridin-3-yl)piperazine (Intermediate EO), 1,3-dimethyl-4-(piperazin-1-yl)-1H-pyrazole-5-carbonitrile piperazine (Intermediate EP), 3-(piperazin-1-yl)thiophene-2-carbonitrile (Intermediate EQ), 4-methyl-5-(piperazin-1-yl)thiazole (Intermediate ER), 1-(1,5-dimethyl-1H-1,2,3-triazol-4-yl)piperazine (Intermediate ES), 5-fluoro-4-(piperazin-1-yl)pyridin-2(1H)-one (Intermediate ET)

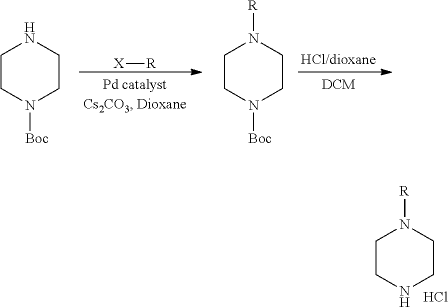

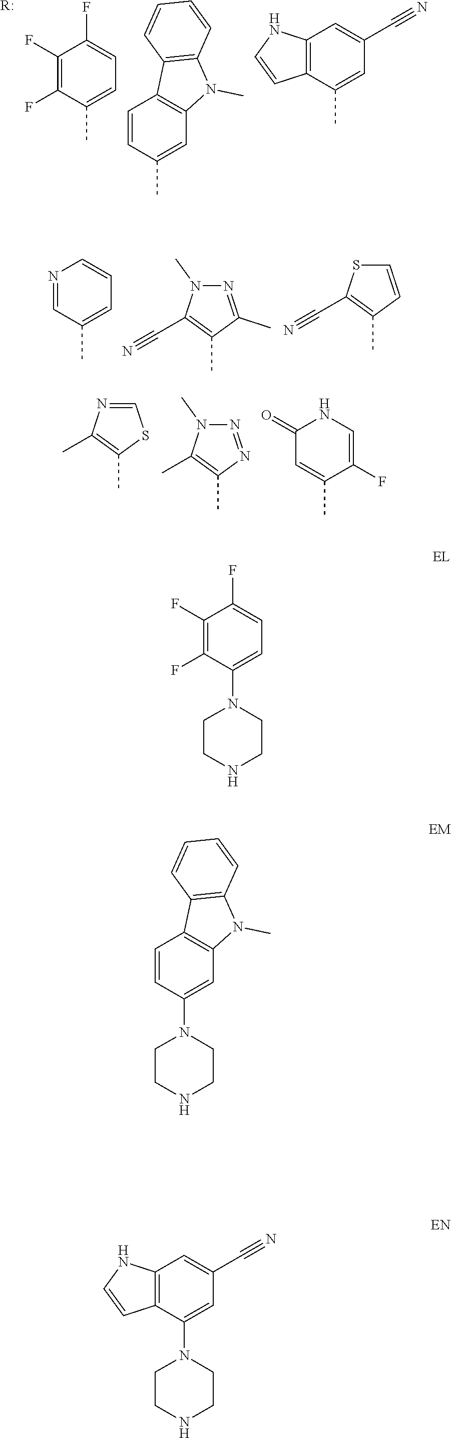

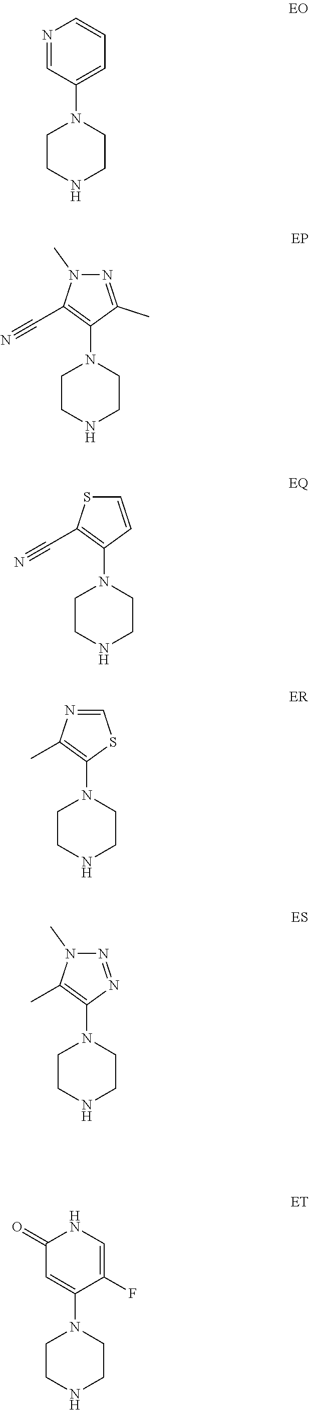

### Step 1

To a mixture of the corresponding Boc-amine (100 mg, 1.0 eq) in dioxane (5 mL) was added reagents halide (R—X, 1.0 eq) and Cs₂CO₃ (2.5 eq). Then catalyst (SP-4-1)-[1,3-BIs[2,6-bis(1-ethylpropyl)phenyl]-4,5-dichloro-1,3-dihydro-2H-imidazol-2-ylidene]dichloro(2-methylpyridine)palladium (CAS #1612891-29-8, 0.1 eq) was added to the mixture at 25° C. under N₂. The mixture was then stirred at 100° C. under N₂ for 12 hrs. On completion, the reaction mixture was diluted with H₂O (20 mL) and then extracted with EtOAc (10 mL×3). The combined organic layers were washed with brine (10 mL), dried over Na₂SO₄, filtered and concentrated under reduced pressure to give the title compounds.

### Step 2

To a mixture of the corresponding piperazines (0.1 g, 1.0 eq) in DCM (2 mL) was added HCl/Dioxane (4 N, 4 mL) at 25° C. The mixture was then stirred at 25° C. for 4 hrs. On completion, the reaction mixture was concentrated under reduced pressure to give the title compounds.

### 2-Amino-2-phenylacetonitrile (CAS #110066-41-6) (Intermediate EU)

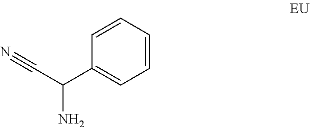

### 1-(Tert-butyl)piperazine (CAS #38216-72-7) (Intermediate EV)

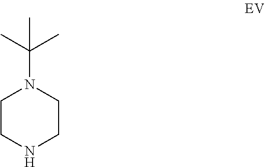

### 4,5,6,7-Tetrahydroisoxazolo[5,4-c]pyridin-3(2H)-one (CAS #64603-91-4) (Intermediate EW)

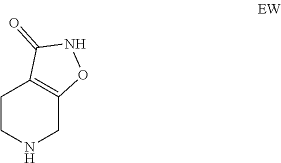

### 1-(2-Cyclopropoxy-6-fluorophenyl)piperazine Intermediate EX

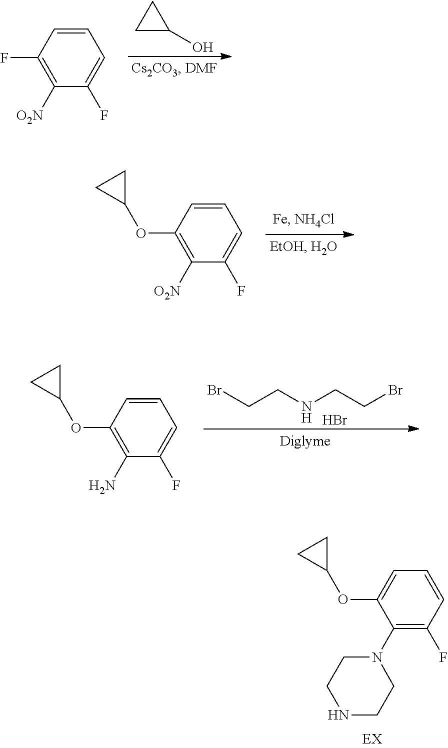

### Step 1—1-Cyclopropoxy-3-fluoro-2-nitrobenzene

A solution of 1,3-difluoro-2-nitrobenzene (1.00 g, 6.29 mmol, CAS #19064-24-5), cyclopropanol (365 mg, 6.29 mmol, CAS #16545-68-9), and Cs₂CO₃ (4.10 g, 12.5 mmol) in DMF (10 mL) was stirred at 80° C. for 12 h under N₂ condition. On completion, the reaction mixture was quenched with water (20 mL) and extracted with ethyl acetate (3×20 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=100/1 to 30/1) to give the title compound (500 mg, 38% yield) as a brown oil. ¹H NMR (400 MHz, DMSO-d₆) δ=7.67 (dt, J=6.8, 8.8 Hz, 1H), 7.45 (d, J=8.8 Hz, 1H), 7.21-7.12 (m, 1H), 4.13 (tt, J=2.8, 6.0 Hz, 1H), 0.88-0.82 (m, 2H), 0.75-0.67 (m, 2H),

### Step 2—2-Cyclopropoxy-6-fluoroaniline

A solution of 1-cyclopropoxy-3-fluoro-2-nitrobenzene (400 mg, 2.03 mmol), NH₄Cl (542 mg, 10.1 mmol), and Fe (566 mg, 10.1 mmol) in EtOH (4 mL) and H₂O (1 mL) was stirred at 80° C. for 2 hrs. On completion, the reaction mixture was filtered and the filtrate was concentrated in vacuo to give the crude residue. The reaction mixture was purified by column chromatography (SiO₂, Petroleum ether:Ethyl acetate=100/1 to 30/1) to give the tittle compound (300 mg, 80% yield) as a yellow oil. ¹H NMR (400 MHz, DMSO-d₆) δ=6.94 (d, J=8.0 Hz, 1H), 6.68 (dd, J=8.8, 10.0 Hz, 1H), 6.57-6.50 (m, 1H), 4.51 (s, 2H), 3.85 (tt, J=3.2, 6.0 Hz, 1H), 0.78-0.73 (m, 2H), 0.71-0.65 (m, 2H).

### Step 3—1-(2-Cyclopropoxy-6-fluorophenyl)piperazine

A solution of 2-cyclopropoxy-6-fluoroaniline (244 mg, 1.46 mmol), bis(2-bromoethyl)amine hydrobromide (683 mg, 2.19 mmol, HBr salt, CAS #3890-99-1) in 2-(2-methoxyethoxy)ethanol (0.5 mL) was stirred at 130° C. for 3 hrs under N₂ condition. On completion, the reaction mixture was purified by reversed-phase HPLC (0.8 g/L ammonium bicarbonate) to give the tittle compound (50.0 mg) as a brown oil. LC-MS (ESI⁺) m/z 237.0 (M+H)⁺.

### 3-Bromo-4-cyclopropoxypyridine (Intermediate EY)

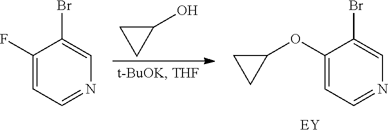

To a solution of 3-bromo-4-fluoropyridine (2 g, 11.36 mmol, CAS #116922-60-2) and cyclopropanol (990 mg, 17.0 mmol, CAS #16545-68-9) in THF (20 mL) was added t-BuOK (3.83 g, 34.0 mmol). The mixture was then stirred at 40° C. for 3 hrs. On completion, the reaction mixture was concentrated under reduced pressure to give a residue. The residue was purified by column chromatography (SiO₂, Petroleum ether:Ethyl acetate=10/1 to 5/1) to give the title compound (2 g, 82% yield) as a white oil. ¹H NMR (400 MHz, DMSO-d₆) δ=8.55 (s, 1H), 8.44 (d, J=5.6 Hz, 1H), 7.41 (d, J=5.6 Hz, 1H), 4.13-4.05 (m, 1H), 0.98-0.91 (m, 2H), 0.73-0.67 (m, 2H).

### (4-Bromo-6-chloro-7-fluoro-1H-indol-2-yl)(4-(3-methoxypyridin-2-yl)piperazin-1-yl)methanone (Intermediate EZ)

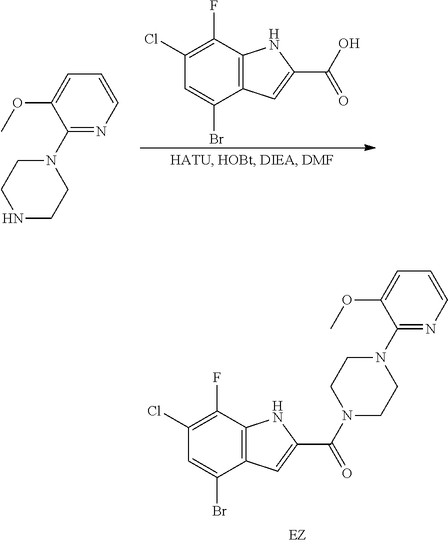 [SMILES: COc1cccnc1N1CCNCC1 ; O=C(O)c1cc2c(Br)cc(Cl)c(F)c2[nH]1 ; COc1cccnc1N1CCN(C(=O)c2cc3c(Br)cc(Cl)c(F)c3[nH]2)CC1]

To a solution of 4-bromo-6-chloro-7-fluoro-1H-indole-2-carboxylic acid (800 mg, 2.74 mmol, synthesized via Steps 1-6 of Intermediate C) in DMF (15 mL) was added DIEA (2.12 g, 16.4 mmol, 2.86 mL), HOBt (739 mg, 5.47 mmol), HATU (1.25 g, 3.28 mmol) and 1-(3-methoxypyridin-2-yl)piperazine (581 mg, 3.01 mmol, CAS #80827-67-4) at 0° C. The reaction was then stirred at 25° C. for 12 hrs. On completion, the crude product was triturated with H₂O at 25° C., then the mixture was filtered and the filter cake was concentrated dried to give the title compound (1.4 g) as a brown solid. LC-MS (ESI⁺) m/z 469.0 (M+H)⁺.

### (6-Chloro-7-fluoro-4-(4-methylpiperazin-1-yl)-1H-indol-2-yl)(4-(3-methoxypyridin-2-yl)piperazin-1-yl)methanone (Intermediate FA)

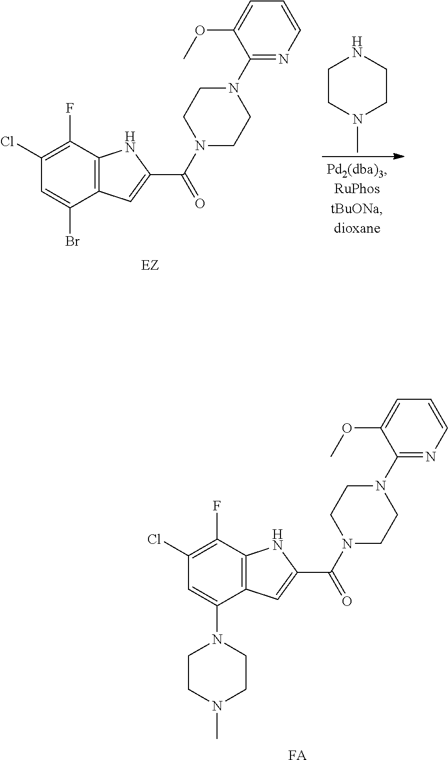

To a solution of (4-bromo-6-chloro-7-fluoro-1H-indol-2-yl)(4-(3-methoxypyridin-2-yl)piperazin-1-yl)methanone (80 mg, 171 μmol, Intermediate EZ), and 1-methylpiperazine (20.5 mg, 205 μmol, 22.7 μL, CAS #109-01-3) in dioxane (10 mL) was added tBuONa (49.3 mg, 513 μmol), RuPhos (7.98 mg, 17.1 μmol), and Pd₂(dba)₃ (15.6 mg, 17.1 μmol). Then the reaction was stirred at 80° C. for 12 hrs under nitrogen atmosphere. On completion, the reaction mixture was diluted with water (10 mL) and extracted with ethyl acetate (3×30 mL). The combined organic layers were washed with brine (10 mL) and dried over anhydrous sodium sulfate, filtered and the filtrate was concentrated in vacuo to give the crude residue. The crude residue was purified by column chromatography (SiO₂, Petroleum ether/Ethyl acetate=1/0 to 0/1) to give the title compound (40 mg, 44% yield) as a yellow solid. LC-MS (ESI⁺) m/z 487.2 (M+H)⁺.

### (6-Chloro-4-cyclohexyl-7-fluoro-1H-indol-2-yl)(4-(3-methoxypyridin-2-yl)piperazin-1-yl)methanone (Intermediate FB)

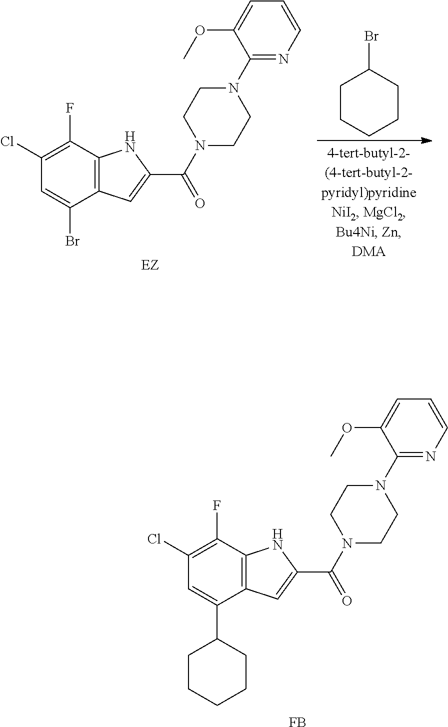

To a solution of (4-bromo-6-chloro-7-fluoro-1H-indol-2-yl)(4-(3-methoxypyridin-2-yl)piperazin-1-yl)methanone (130 mg, 277 μmol, Intermediate EZ) in DMA (2 mL) was added bromocyclohexane (67.9 mg, 416 μmol, 51.3 μL, CAS #108-85-0), diiodonickel (8.69 mg, 27.7 μmol, 1.49 μL), MgCl₂ (26.4 mg, 277 μmol, 11.4 μL), Bu₄NI (102 mg, 277 μmol) and 4-tert-butyl-2-(4-tert-butyl-2-pyridyl)pyridine (7.46 mg, 27.7 μmol). Then the reaction was stirred at 25° C. for 12 hrs. On completion, the reaction mixture was concentrated in vacuo to give the crude residue. The crude residue was purified by reversed-phase HPLC (0.1% FA condition) to give the title compound (80 mg, 56% yield) as a brown solid. LC-MS (ESI⁺) m/z 471.2 (M+H)⁺.

### (2-(Trifluoromethoxy)phenyl)boronic acid (CAS #175676-65-0) (Intermediate FC)

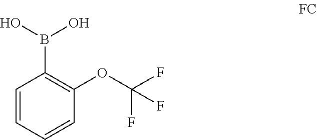 [SMILES: OB(O)c1ccccc1OC(F)(F)F]

### (4,5-Difluoro-2-methoxyphenyl)boronic acid (CAS #870777-32-5) (Intermediate FD)

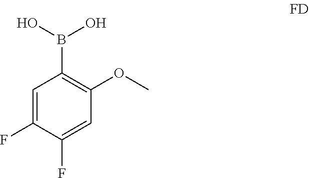

### (2-Methoxy-3-methylphenyl)boronic acid (CAS #909187-39-9) (Intermediate FE)

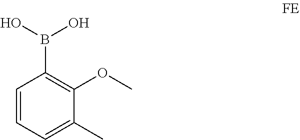

### 3-Bromo-4-ethyl-pyridine (CAS #38749-76-7) (Intermediate FG)

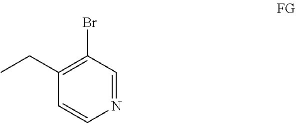

### (2-Ethylphenyl)boronic acid (CAS #90002-36-1) (Intermediate FH)

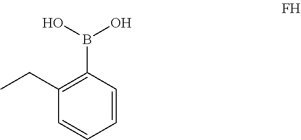

### 1-(5-(4-Chloro-7-fluoro-2-(4-(3-fluoro-2-methoxyphenyl)piperazine-1-carbonyl)-1H-indol-6-yl)-3,6-dihydropyridin-1(2H)-yl)-3-(1H-1,2,3-triazol-1-yl)propan-1-one (Intermediate FI)

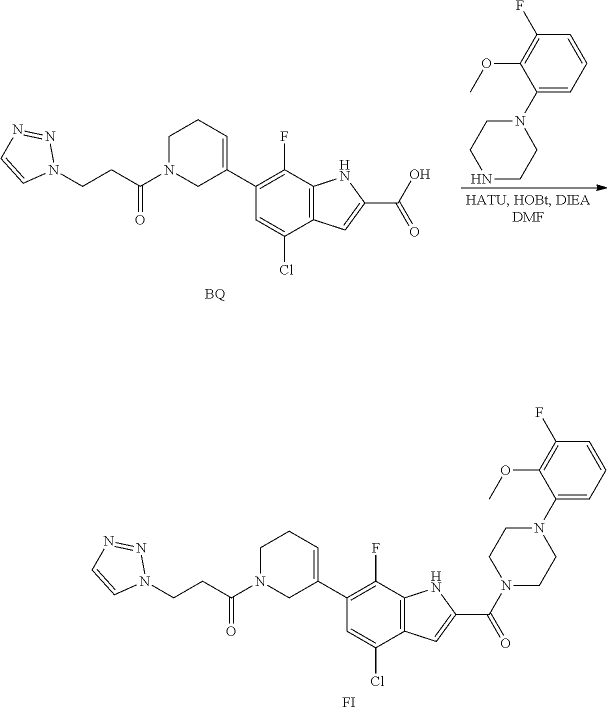 [SMILES: O=C(O)c1cc2c(Cl)cc(C3=CCCN(C(=O)CCn4ccnn4)C3)c(F)c2[nH]1 ; COc1c(F)cccc1N1CCNCC1 ; COc1c(F)cccc1N1CCN(C(=O)c2cc3c(Cl)cc(C4=CCCN(C(=O)CCn5ccnn5)C4)c(F)c3[nH]2)CC1 ; "FI"]

To a solution of 6-(1-(3-(1H-1,2,3-triazol-1-yl)propanoyl)-1,2,5,6-tetrahydropyridin-3-yl)-4-chloro-7-fluoro-1H-indole-2-carboxylic acid (150 mg, 359 μmol, Intermediate BQ) in DMF (2 mL) was added HOBt (53.36 mg, 394 μmol), HATU (177 mg, 466 μmol), DIEA (185 mg, 1.44 mmol, 250 μL) and 1-(3-fluoro-2-methoxyphenyl)piperazine (105 mg, 502 μmol, CAS #1121613-46-4). Then the reaction was stirred at 25° C. for 10 min. On completion, the reaction mixture was diluted with water (20 mL), then the reaction mixture was filtered and the filter cake was dried in vacuo to give the title compound (200 mg) as a yellow solid. LC-MS (ESI⁺) m/z 610.2 (M+H)⁺.

### 3-Methoxy-1′,2′,3′,6′-tetrahydro-2,4′-bipyridine (Intermediate FJ)

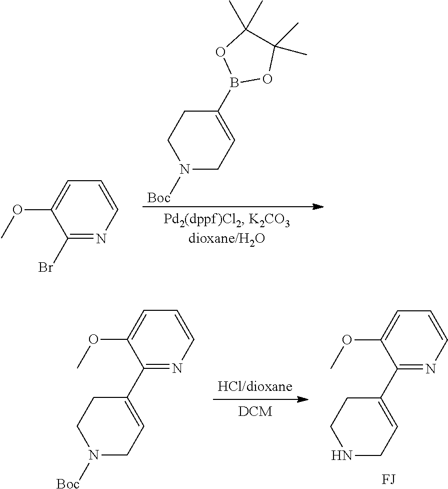

### Step 1—Tert-butyl 3-methoxy-5′,6′-dihydro-[2,4′-bipyridine]-1′(2′H)-carboxylate

A mixture of 2-bromo-3-methoxy-pyridine (1 g, 5 mmol, CAS #24100-18-3), tert-butyl 4-(4,4,5,5-tetramethyl-1,3,2-dioxaborolan-2-yl)-3,6-dihydro-2H-pyridine-1-carboxylate (1.64 g, 5.32 mmol, CAS #286961-14-6), K₂CO₃ (2.21 g, 16.0 mmol), and Pd(dppf)Cl₂ (389 mg, 532 μmol) in dioxane (16 mL) and H₂O (4 mL) was degassed and purged with N₂ three times. Then the mixture was stirred at 80° C. for 2 hrs under N₂ atmosphere. On completion, the reaction mixture was concentrated under reduced pressure to give a residue. The residue was purified by column chromatography (SiO₂, Petroleum ether/Ethyl acetate=1/0 to 2/1) to give the title compound (1.2 g, 74% yield) as a yellow oil. LC-MS (ESI⁺) m/z 291.0 (M+H)⁺; ¹H NMR (400 MHz, DMSO-d₆) δ=8.12 (dd, J=0.8, 4.4 Hz, 1H), 7.44 (d, J=8.0 Hz, 1H), 7.25 (dd, J=4.4, 8.4 Hz, 1H), 6.51 (s, 1H), 4.02 (s, 2H), 3.82 (s, 3H), 3.50 (t, J=5.6 Hz, 2H), 2.54 (d, J=1.6 Hz, 2H), 1.43 (s, 9H).

### Step 2—3-Methoxy-1′,2′,3′,6′-tetrahydro-2,4′-bipyridine

To a solution of tert-butyl 4-(3-methoxy-2-pyridyl)-3,6-dihydro-2H-pyridine-1-carboxylate (200 mg, 689 μmol) in DCM (3 mL) was added HCl/dioxane (2 M, 3 mL). The mixture was stirred at 25° C. for 1 hr. On completion, the reaction mixture was concentrated under reduced pressure to the title compound (300 mg, HCl) as a yellow oil. LC-MS (ESI⁺) m/z 191.1 (M+H)⁺.

### 4-Chloro-5-methoxypyridazine (Intermediate FK)

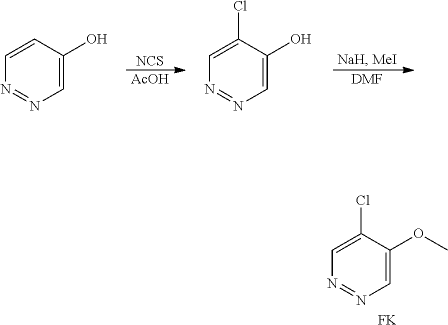

### Step 1—5-Chloropyridazin-4-ol

To a solution of pyridazin-4-ol (5 g, 50 mmol, CAS #20733-10-2) in AcOH (50 mL) was added NCS (6.95 g, 52.0 mmol). The mixture was then stirred at 60° C. for 4 hrs. On completion, the mixture was concentrated under reduced pressure to remove solvent. Then the residue was purified by column chromatography (SiO₂, DCM:MeOH=10:1) to give the title compound (4 g, 53% yield) as a white solid. ¹H NMR (400 MHz, DMSO-d₆) δ=8.89-8.77 (m, 1H), 7.99-7.89 (m, 1H); LC-MS (NEG) m/z 129.0 (M−H)⁺.

### Step 2—4-Chloro-5-methoxypyridazine

To a solution of 5-chloropyridazin-4-ol (200 mg, 1.53 mmol) in DMF (2 mL) was added K₂CO₃ (635 mg, 4.60 mmol) and MeI (261 mg, 1.84 mmol). The mixture was then stirred at 80° C. for 12 hrs. On completion, the reaction mixture was concentrated under reduced pressure to remove solvent. Then the residue was purified by column chromatography (SiO₂, Petroleum ether/Ethyl acetate=0/1) to give the title compound (150 mg, 61% yield) as a yellow solid. ¹H NMR (400 MHz, DMSO-d₆) δ=8.95-8.85 (m, 1H), 7.97-7.87 (m, 1H), 3.93 (d, J=1.6 Hz, 3H).

### 3-Bromo-4-(difluoromethoxy)pyridine (Intermediate FL)

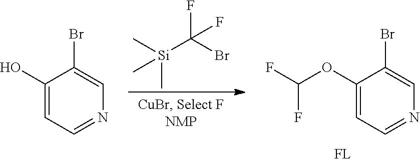

To a solution of 3-bromopyridin-4-ol (200 mg, 1.15 mmol, CAS #36953-41-0), (bromodifluoromethyl)trimethylsilane (700 mg, 3.45 mmol, CAS #115262-01-6), CuBr (494 mg, 3.45 mmol), and Select F (814 mg, 2.30 mmol) in NMP (2 mL) was stirred at 25° C. for 12 hrs. On completion, the reaction mixture was quenched with sat. NH₄HCO₃ (20 mL) and extracted with ethyl acetate (3×20 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=100/1 to 10/1) to give the title compound (50 mg) as a white solid. ¹H NMR (400 MHz, DMSO-d₆) δ=8.62 (d, J=2.4 Hz, 1H), 8.09 (dd, J=2.4, 8.0 Hz, 1H), 7.70-7.37 (m, 1H), 6.39 (d, J=8.0 Hz, 1H).

### Methyl 6-(1-(3-(1H-1,2,3-triazol-1-yl)propanoyl)-1,2,5,6-tetrahydropyridin-3-yl)-7-fluoro-4-(4,4,5,5-tetramethyl-1,3,2-dioxaborolan-2-yl)-1H-indole-2-carboxylate (Intermediate FM)

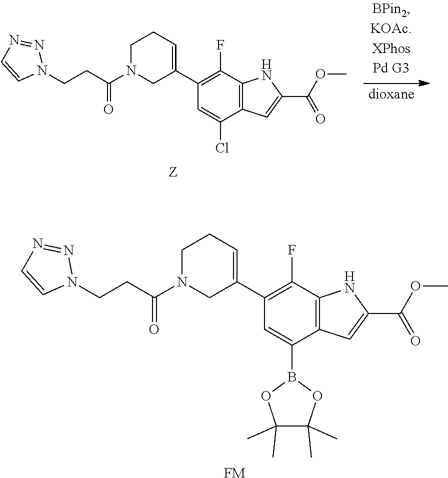

A mixture of methyl 6-(1-(3-(1H-1,2,3-triazol-1-yl)propanoyl)-1,2,5,6-tetrahydropyridin-3-yl)-4-chloro-7-fluoro-1H-indole-2-carboxylate (5.00 g, 11.5 mmol, Intermediate Z), 4,4,5,5-tetramethyl-2-(4,4,5,5-tetramethyl-1,3,2-dioxaborolan-2-yl)-1,3,2-dioxaborolane (8.82 g, 34.7 mmol), XPhos Pd G3 (980 mg, 1.16 mmol), and KOAc (3.41 g, 34.7 mmol) in dioxane (50 mL) was degassed and purged with N₂ three times. Then the mixture was stirred at 90° C. for 2 hrs under N₂ atmosphere. On completion, the crude residue was triturated with water at 25° C. for 0.5 hrs. Then the mixture was filtered and the filter cake was concentrated in vacuo to give the title compound (2.40 g) as a yellow solid. LC-MS (ESI⁺) m/z 524.0 (M+H)⁺; ¹H NMR (400 MHz, DMSO-d₆) δ=12.53 (d, J=4.0 Hz, 1H), 8.17-8.07 (m, 1H), 7.73-7.64 (m, 1H), 7.44-7.34 (m, 2H), 6.07 (s, 1H), 4.67-4.58 (m, 2H), 4.34-4.20 (m, 2H), 3.92-3.86 (m, 3H), 3.67-3.54 (m, 2H), 3.13-3.01 (m, 2H), 2.38-2.24 (m, 2H), 1.34 (s, 12H).

### 6-(1-(3-(1H-1,2,3-triazol-1-yl)propanoyl)-1,2,5,6-tetrahydropyridin-3-yl)-4-(4-(difluoromethoxy)pyridin-3-yl)-7-fluoro-1H-indole-2-carboxylic acid (Intermediate FN)

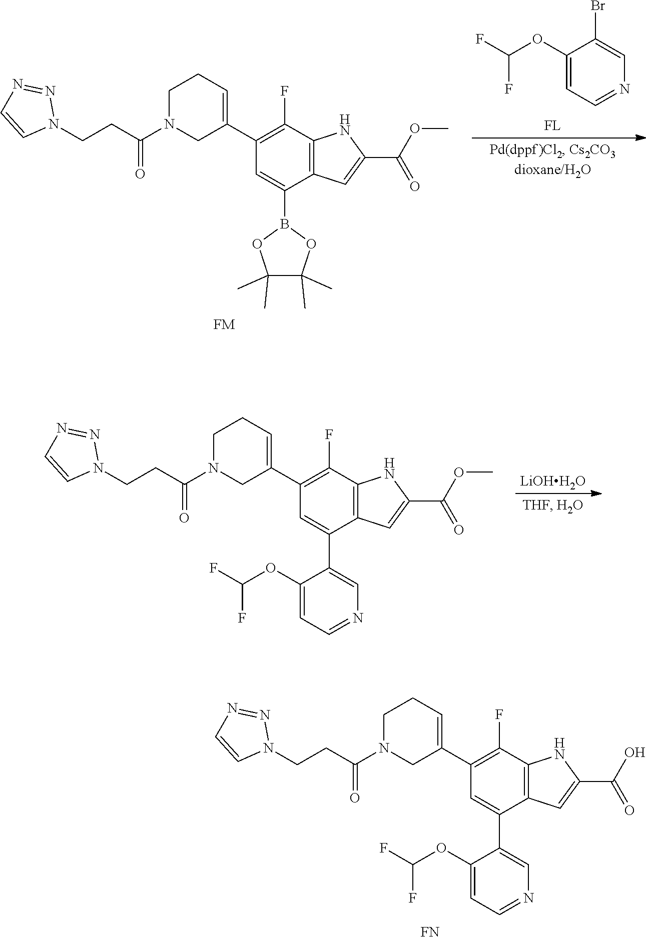

### Step 1—Methyl 6-(1-(3-(1H-1,2,3-triazol-1-yl)propanoyl)-1,2,5,6-tetrahydropyridin-3-yl)-4-(4-(difluoromethoxy)pyridin-3-yl)-7-fluoro-1H-indole-2-carboxylate

A solution of 3-bromo-4-(difluoromethoxy)pyridine (48.1 mg, 214 μmol, Intermediate FL), methyl 6-(1-(3-(1H-1,2,3-triazol-1-yl)propanoyl)-1,2,5,6-tetrahydropyridin-3-yl)-7-fluoro-4-(4,4,5,5-tetramethyl-1,3,2-dioxaborolan-2-yl)-1H-indole-2-carboxylate (75.0 mg, 143 μmol, Intermediate FM), Cs₂CO₃ (140 mg, 429 μmol), and Pd(dppf)Cl₂·CH₂Cl₂ (11.7 mg, 14.3 μmol) in dioxane (1 mL) and H₂O (0.4 mL) was stirred at 80° C. for 2 hrs. On completion, the crude residue was purified by reversed-phase HPLC (0.8 g/L ammonium bicarbonate) to give the tittle compound (80.0 mg, 90% yield) as a pink solid. LC-MS (ESI⁺) m/z 541.3 (M+H)⁺.

### Step 2—6-(1-(3-(1H-1,2,3-triazol-1-yl)propanoyl)-1,2,5,6-tetrahydropyridin-3-yl)-4-(4-(difluoromethoxy)pyridin-3-yl)-7-fluoro-1H-indole-2-carboxylic acid

A solution of methyl 6-(1-(3-(1H-1,2,3-triazol-1-yl)propanoyl)-1,2,5,6-tetrahydropyridin-3-yl)-4-(4-(difluoromethoxy)pyridin-3-yl)-7-fluoro-1H-indole-2-carboxylate (50.0 mg, 92.5 μmol) and LiOH·H₂O (19.4 mg, 462 μmol) in THE (1 mL) and H₂O (1 mL) was stirred at 25° C. for 2 hrs. On completion, the crude residue was purified by reversed-phase HPLC (0.1% FA condition) to give the title compound (46.0 mg, 83% yield, FA) as a brown solid. LC-MS (ESI⁺) m/z 527.0 (M+H)⁺.

### 4-Methoxy-3-(4,4,5,5-tetramethyl-1,3,2-dioxaborolan-2-yl)pyridine (CAS #758699-74-0) (Intermediate FO)

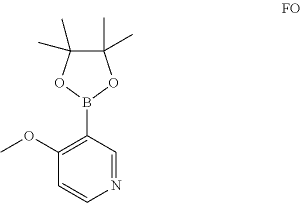

### 1-(5-(4-chloro-2-(4-(3-ethoxypyridin-2-yl)piperazine-1-carbonyl)-7-fluoro-1H-indol-6-yl)-3,6-dihydropyridin-1(2H)-yl)-3-(1H-1,2,3-triazol-1-yl)propan-1-one (Intermediate FP)

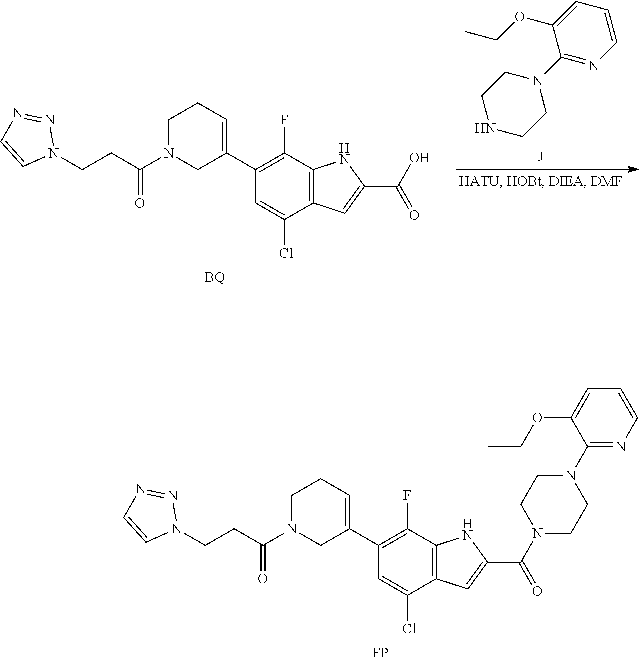 [SMILES: O=C(O)c1cc2c(Cl)cc(C3=CCCN(C(=O)CCn4ccnn4)C3)c(F)c2[nH]1 ; CCOc1cccnc1N1CCNCC1 ; CCOc1cccnc1N1CCN(C(=O)c2cc3c(Cl)cc(C4=CCCN(C(=O)CCn5ccnn5)C4)c(F)c3[nH]2)CC1 ; "FP"]

To a solution of 1-(3-ethoxy-2-pyridyl)piperazine (79.4 mg, 287 μmol, Intermediate J), and 4-chloro-7-fluoro-6-[1-[3-(triazol-1-yl)propanoyl]-3,6-dihydro-2H-pyridin-5-yl]-1H-indole-2-carboxylic acid (120 mg, 287 μmol, Intermediate BQ) in DMF (2 mL) was added HATU (131 mg, 345 μmol), HOBt (46.6 mg, 345 μmol) and DIEA (186 mg, 1.44 mmol, 250 μL). The mixture was then stirred at 25° C. for 0.5 hr. On completion, the reaction mixture was quenched by addition of H₂O (10 mL) at 0° C., and then diluted with EA (20 mL) and extracted with EA (30 mL×2). The combined organic layers were washed with NaCl (50 mL×2), dried over Na₂SO₄, filtered and concentrated under reduced pressure to give a residue. The residue was purified by column chromatography (SiO₂, Petroleum ether/Ethyl acetate=10/1 to 1/5) to give the title compound (130 mg, 40% yield) as a white solid. LC-MS (ESI⁺) m/z 607.3 (M+H)⁺.

### (4-Methoxy-3-pyridyl)boronic acid (CAS #355004-67-0) (Intermediate FQ)

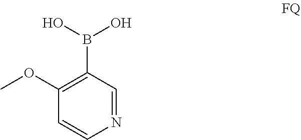

### 2-Methyl-1-(pyridin-2-yl)piperazine (Intermediate FR)

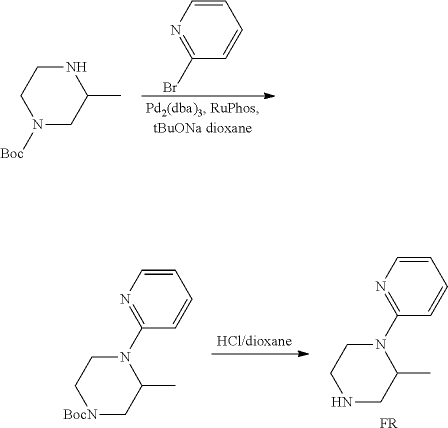

### Step 1—Tert-butyl 3-methyl-4-(pyridin-2-yl)piperazine-1-carboxylate

A mixture of tert-butyl 3-methylpiperazine-1-carboxylate (761 mg, 3.80 mmol), BrCl=NC═CC=Cl (0.5 g, 3.16 mmol, 302 μL), Pd₂(dba)₃ (290 mg, 316 μmol), RuPhos (148 mg, 316 μmol) and tBuONa (912 mg, 9.49 mmol) in dioxane (10 mL) was degassed and purged with N₂ three times. Then the mixture was stirred at 80° C. for 12 hrs under N₂ atmosphere. On completion, the reaction mixture was quenched with H₂O (20 mL), and then diluted with EA (20 mL) and extracted with EA (50 mL×2). The combined organic layers were washed with NaCl (50 mL×2), dried over NaCl, filtered and concentrated under reduced pressure to give a residue. The residue was purified by column chromatography (SiO₂, Petroleum ether/Ethyl acetate=50/1 to 5/1) to give the title compound (600 mg, 67% yield) as a white solid. LC-MS (ESI⁺) m/z 278.1 (M+H)⁺.

### Step 2—2-Methyl-1-(pyridin-2-yl)piperazine

To a solution of tert-butyl 3-methyl-4-(2-pyridyl)piperazine-1-carboxylate (150 mg, 541 mol) in DCM (0.5 mL) was added HCl/dioxane (2 M, 270 μL). The mixture was then stirred at 25° C. for 1 hr. On completion, the reaction mixture was filtered and concentrated under reduced pressure to give the title compound (90 mg) as a white solid. LC-MS (ESI⁺) m/z 179.3 (M+H)⁺.

### 1-(5-(4-Chloro-7-fluoro-2-(3-methyl-4-(pyridin-2-yl)piperazine-1-carbonyl)-1H-indol-6-yl)-3,6-dihydropyridin-1(2H)-yl)-3-(1H-1,2,3-triazol-1-yl)propan-1-one (Intermediate FS)

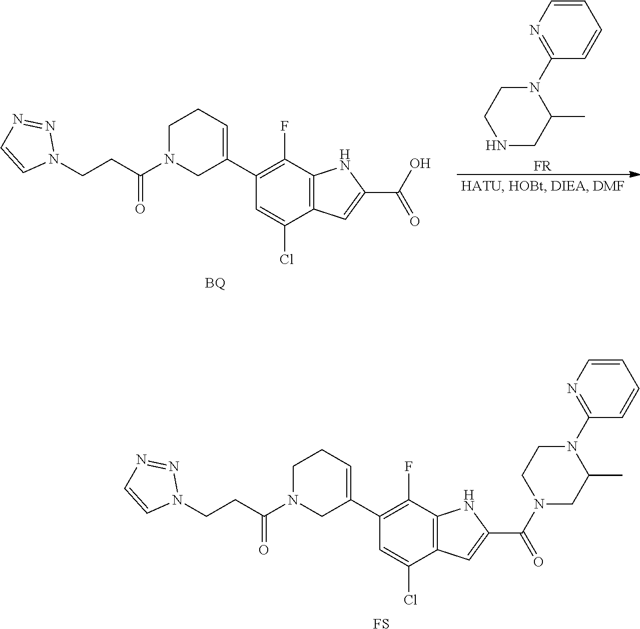

To a solution of 2-methyl-1-(2-pyridyl)piperazine (84.8 mg, 359 μmol, Intermediate FR) 4-chloro-7-fluoro-6-[1-[3-(triazol-1-yl)propanoyl]-3,6-dihydro-2H-pyridin-5-yl]-1H-indole-2-carboxylic acid (150 mg, 359 μmol, Intermediate BQ) in DMF (2 mL) was added HOBt (58.2 mg, 431 μmol), HATU (164 mg, 431 μmol), and DIEA (232 mg, 1.80 mmol, 313 μL). Then the mixture was stirred at 25° C. for 0.5 hr. On completion, the reaction mixture was quenched with H₂O (10 mL) at 0° C., and then diluted with EA (20 mL) and extracted with EA (30 mL×2). The combined organic layers were washed with NaCl (50 mL×2), dried over Na₂SO₄, filtered and concentrated under reduced pressure to give a residue. The residue was purified by column chromatography (SiO₂, Petroleum ether/Ethyl acetate=10/1 to 1/5) to give the title compound (180 mg, 48% yield) as a white solid. LC-MS (ESI⁺) m/z 577.2 (M+H)⁺.

### 2-Methylcyclohex-1-en-1-yl trifluoromethanesulfonate (Intermediate FT)

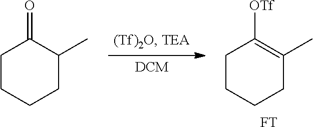

A solution of 2-methylcyclohexanone (0.5 g, 4.46 mmol, 541 μL, CAS #583-60-8), Tf₂O (1.51 g, 5.35 mmol, 882 μL), and TEA (902 mg, 8.92 mmol, 1.24 mL) in DCM (8 mL) was stirred at 0° C. for 12 hrs. On completion, the reaction mixture was quenched with water (20 mL) and extracted with ethyl acetate (3×20 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=100/1 to 10/1) to give the title compound (200 mg) as a yellow oil. ¹H NMR (400 MHz, DMSO-d₆) δ=5.87-5.82 (m, 1H), 2.27 (tdd, J=2.0, 4.0, 6.0 Hz, 2H), 2.13 (t, J=6.0 Hz, 2H), 1.70 (s, 3H), 1.69-1.66 (m, 1H), 1.59-1.51 (m, 2H), 1.48-1.37 (m, 1H), 1.21-1.05 (m, 1H).

### 1-(3-(Difluoromethoxy)phenyl)piperazine (Intermediate FU)

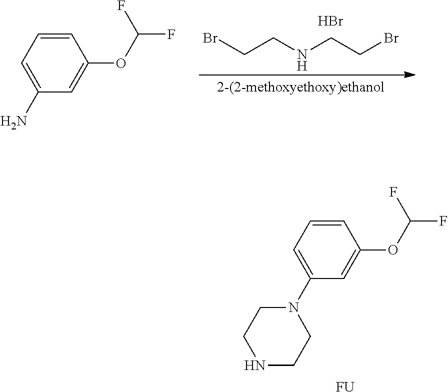

To a solution of 3-(difluoromethoxy)aniline (1.09 g, 4.71 mmol, CAS #22236-08-4) in 2-(2-methoxyethoxy) ethanol (1 mL) was added bis(2-bromoethyl)amine hydrobromide (500 mg, 3.14 mmol, CAS #3890-99-1). Then the mixture was stirred at 130° C. for 4 hrs. On completion, the reaction mixture was triturated with acetone at 0° C. for 15 min, and filtered and dried to give the title compound (500 mg) as a black solid. TLC (PE/EA=0/1, Rf=0.05) indicated started material was consumed completely and one new spot formed.

### 5-Bromo-4-methoxy-2-methyl-pyridine (CAS #886372-61-8) (Intermediate FV)

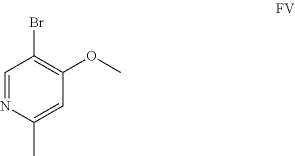

### 4-Bromo-3-methoxy-aniline (CAS #19056-40-7) (Intermediate FW)

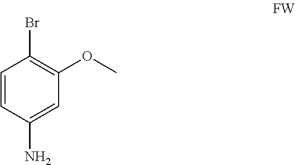

### 5-Methoxy-4-(piperazin-1-yl)pyrimidine (CAS #141071-86-5) (Intermediate FX)

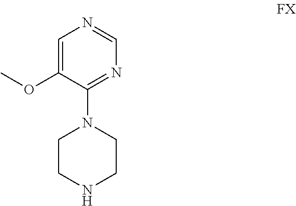

### 4-Bromo-2,5-dimethyl-1H-imidazole (Intermediate FY)

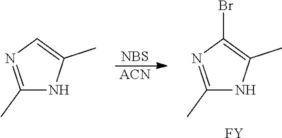

To a solution of 2,5-dimethyl-1H-imidazole (0.2 g, 2.1 mmol, CAS #930-62-1) in ACN (2 mL) was added NBS (741 mg, 4.16 mmol). The mixture was then stirred at 25° C. for 2 hrs. On completion, the reaction mixture was quenched by addition of H₂O (10 mL), and then diluted with EA (10 mL) and extracted with EA (20 mL×2). The combined organic layers were washed with NaCl (20 mL×2), dried over Na₂SO₄, filtered and concentrated under reduced pressure to give a residue. The residue was purified by column chromatography (SiO₂, Petroleum ether/Ethyl acetate=10/1 to 1/5) to give the title compound (180 mg, 42% yield) as a white solid. LC-MS (ESI⁺) m/z 175.0 (M+H)⁺.

### (2-Cyanophenyl)boronic acid (CAS #138642-62-3) (Intermediate FZ)

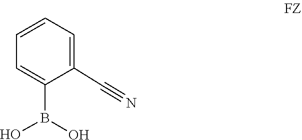 [SMILES: N#Cc1ccccc1B(O)O]

### (1,3-Dimethylpyrazol-4-yl)boronic acid (CAS #1146616-03-6) (Intermediate GA)

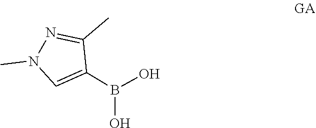

### 6-Bromopyridine-3-carbonitrile (CAS #139585-70-9) (Intermediate GB)

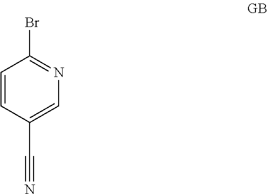

### (4-Cyano-2-methoxyphenyl)boronic acid (CAS #1256345-67-1) (Intermediate GC)

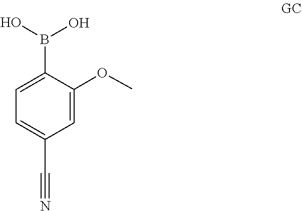

### 6-Bromo-5-methoxyquinoline (Intermediate GD)

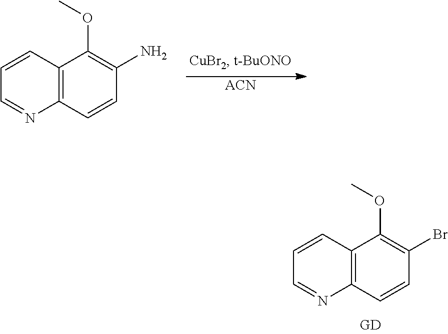

A solution of 5-methoxyquinolin-6-amine (700 mg, 4.02 mmol, CAS #54620-48-3), CuBr₂ (1.08 g, 4.82 mmol), and t-BuONO (621 mg, 6.03 mmol) in ACN (5 mL) was stirred at 25° C. for 2 hrs. On completion, the reaction mixture was filtered and the filter cake was concentrated in vacuo to give the title compound (500 mg) as a brown solid. LC-MS (ESI⁺) m/z 237.9 (M+H)⁺.

### (6′-Chloro-7′-fluoro-1′H-[1,4′-biindol]-2′-yl)(4-(3-methoxypyridin-2-yl)piperazin-1-yl)methanone (Intermediate GE)

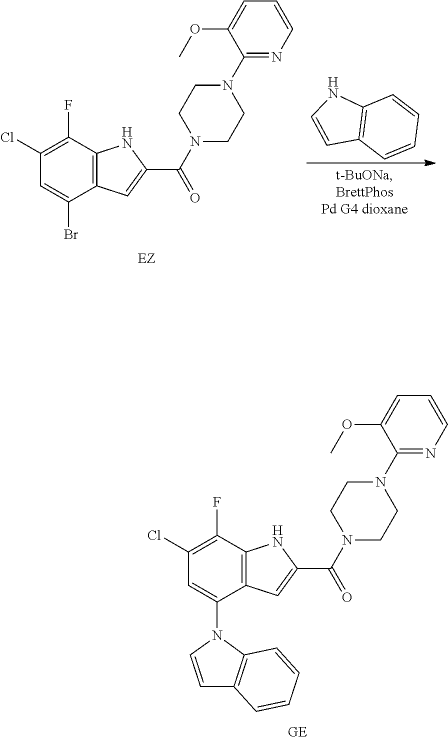 [SMILES: COc1cccnc1N1CCN(C(=O)c2cc3c(Br)cc(Cl)c(F)c3[nH]2)CC1 ; c1ccc2[nH]ccc2c1 ; COc1cccnc1N1CCN(C(=O)c2cc3c(-n4ccc5ccccc54)cc(Cl)c(F)c3[nH]2)CC1]

A mixture of (4-bromo-6-chloro-7-fluoro-1H-indol-2-yl)(4-(3-methoxypyridin-2-yl)piperazin-1-yl)methanone (150 mg, 321 μmol, Intermediate EZ), 1H-indole (37.6 mg, 321 μmol, CAS #120-72-9), t-BuONa (2 M, 48.1 μL) and BrettPhos Pd G4 (29.5 mg, 32.1 μmol) in dioxane (4 mL) was degassed and purged with N₂ three times. Then the mixture was stirred at 90° C. for 12 hrs under N₂ atmosphere. On completion, the residue was purified by column chromatography (SiO₂, Petroleum ether: Ethyl acetate=10/1 to 1/1) to give the title compound (65 mg, 37% yield) as a yellow solid. LC-MS (ESI⁺) m/z 504.1 (M+H)⁺.

### (6-Chloro-7-fluoro-4-(4,4,5,5-tetramethyl-1,3,2-dioxaborolan-2-yl)-1H-indol-2-yl)(4-(5-fluoro-3-methoxypyridin-2-yl)piperazin-1-yl)methanone (Intermediate GF)

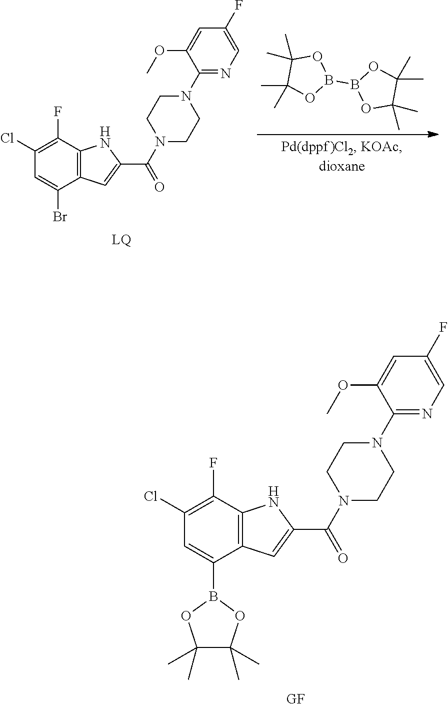

A mixture of (4-bromo-6-chloro-7-fluoro-1H-indol-2-yl)-[4-(5-fluoro-3-methoxy-2-pyridyl)piperazin-1-yl]methanone (500 mg, 1.03 mmol, Intermediate LQ), 4,4,5,5-tetramethyl-2-(4,4,5,5-tetramethyl-1,3,2-dioxaborolan-2-yl)-1,3,2-dioxaborolane (522.82 mg, 2.06 mmol), KOAc (303.09 mg, 3.09 mmol), and Pd(dppf)Cl₂·CH₂Cl₂ (84.07 mg, 102.9 μmol) in dioxane (10 mL) was degassed and purged with N₂ three times. Then the mixture was stirred at 90° C. for 12 hrs under N₂ atmosphere. On completion, the mixture was diluted with ethyl acetate (20 mL) and water (20 mL), and the mixture was extracted with ethyl acetate (3×20 mL). The combined organic extracts were washed with brine (20 mL) and then dried over anhydrous sodium sulfate, filtered and concentrated under vacuum to give a residue. The residue was purified by flash silica gel chromatography (ISCO®; 20 g SepaFlash® Silica Flash Column, Eluent of 0˜35% Ethyl acetate/Petroleum ether gradient @40 mL/min) to give the title compound (350 mg, 46% yield) as yellow solid. ¹H NMR (400 MHz, DMSO-d₆) δ 12.40 (s, 1H), 7.80 (d, J=2.4 Hz, 1H), 7.43-7.34 (m, 2H), 7.04-6.98 (m, 1H), 3.85 (s, 3H), 3.80-3.74 (m, 4H), 3.30-3.27 (m, 4H), 1.33 (s, 12H).

### 4-Bromo-3-methoxy-N-methylaniline (Intermediate GG)

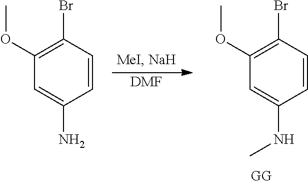

To a solution of 4-bromo-3-methoxyaniline (2 g, 10 mmol, CAS #19056-40-7) in DMF (10 mL) was added NaH (395 mg, 9.90 mmol, 60% dispersion in mineral oil) under N₂ atmosphere at 0° C. The mixture was stirred at 0° C. for 0.5 hrs, and then MeI (1.55 g, 10.8 mmol, 677 μL) was added at 0° C. The resulting mixture was stirred at 25° C. for 4 hrs. On completion, the reaction mixture was quenched with sat. NH₄Cl (20 mL) and extracted with ethyl acetate (3×50 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 10/1) to give the title compound (900 mg, 39% yield) as a yellow oil. ¹H NMR (400 MHz, Chloroform-d) δ=7.26 (d, J=8.4 Hz, 1H), 6.15 (d, J=2.4 Hz, 1H), 6.10 (dd, J=2.4, 8.4 Hz, 1H), 3.84 (s, 3H), 2.81 (s, 3H).

### 1-(6-(Trifluoromethoxy)pyridin-2-yl)piperazine (Intermediate GH)

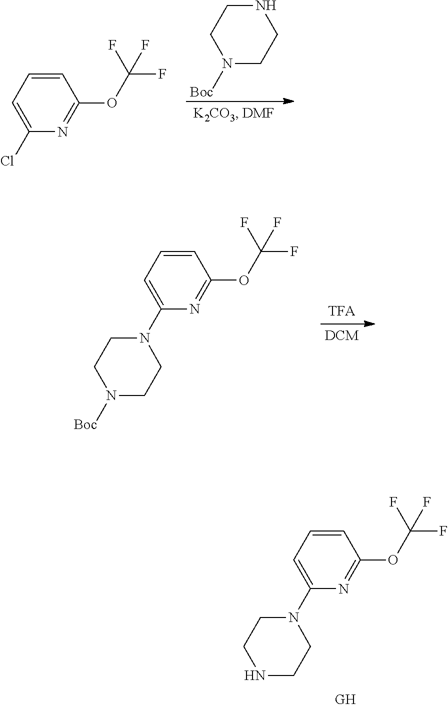 [SMILES: FC(F)(F)Oc1cccc(Cl)n1 ; CC(C)(C)OC(=O)N1CCNCC1 ; CC(C)(C)OC(=O)N1CCN(c2cccc(OC(F)(F)F)n2)CC1 ; FC(F)(F)Oc1cccc(N2CCNCC2)n1]

### Step 1—Tert-butyl 4-(6-(trifluoromethoxy)pyridin-2-yl)piperazine-1-carboxylate

A mixture of 2-chloro-6-(trifluoromethoxy)pyridine (1 g, 5 mmol, CAS #1221171-70-5), tert-butyl piperazine-1-carboxylate (1.13 g, 6.07 mmol, CAS #57260-71-6) and K₂CO₃ (2.10 g, 15.1 mmol) were taken up into a microwave tube in DMF (10 mL). The sealed tube was heated at 130° C. for 2 hrs under microwave. On completion, the reaction mixture was concentrated under reduced pressure to give a residue and purified by prep-HPLC (FA condition) to give the title compound (0.2 g, 9% yield, FA) as a white solid. LC-MS (ESI⁺) m/z 292.0 (M−56)⁺.

### Step 2—1-(6-(Trifluoromethoxy)pyridin-2-yl)piperazine

A mixture of To a solution of tert-butyl 4-(6-(trifluoromethoxy)pyridin-2-yl)piperazine-1-carboxylate (200 mg, 575 μmol) in DCM (2 mL) was added TFA (65.6 mg, 575 μmol, 42.7 μL). The mixture was then stirred at 25° C. for 2 hrs. On completion, the reaction mixture was filtered and the filtrate was concentrated in vacuo to give the title compound (140 mg, TFA) as a red oil. LC-MS (ESI⁺) m/z 248.0 (M+H)⁺.

### 1-(4-Fluoro-2-methoxyphenyl)piperazine (Intermediate GI)

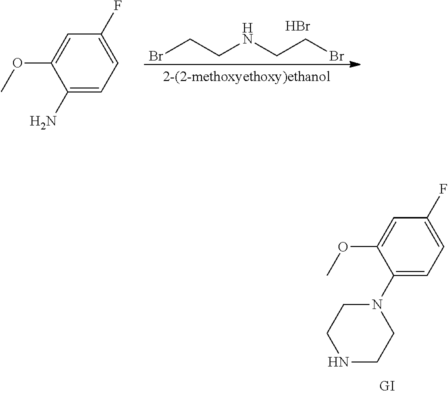

A solution of 4-fluoro-2-methoxy-aniline (500 mg, 3.54 mmol, CAS #450-91-9), and 2-bromo-N-(2-bromoethyl)ethanamine hydrobromide (1.66 g, 5.31 mmol, CAS #43204-63-3) in 2-(2-methoxyethoxy)ethanol (0.500 mL) was stirred at 130° C. for 4 hrs under nitrogen atmosphere. On completion, the reaction mixture was concentrated under reduced pressure The crude product was purified by reversed-phase HPLC(0.1% HCl condition) to give the title compound (300 mg, 33% yield, HCl) as a yellow oil. LC-MS (ESI⁺) m/z 211.1 (M+H)⁺; ¹H NMR (400 MHz, DMSO-d₆) δ=7.02-6.93 (m, 1H), 6.90 (dd, J=2.8, 10.8 Hz, 1H), 6.71 (dt, J=2.8, 8.4 Hz, 1H), 3.80 (s, 3H), 3.17 (s, 4H), 3.16 (s, 4H), 1.36-0.76 (m, 1H).

### 1-(2,4-Difluoro-6-methoxy-phenyl)piperazine (Intermediate GJ)

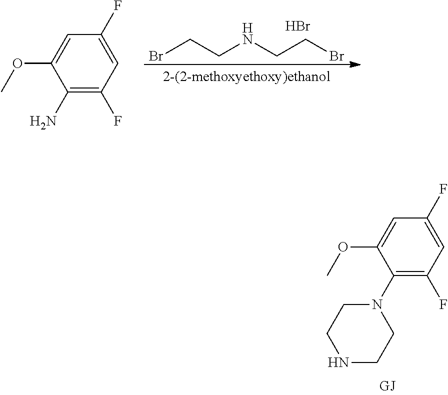

A solution of 2,4-difluoro-6-methoxy-aniline (500 mg, 3.14 mmol) and 2-bromo-N-(2-bromoethyl)ethanamine hydrobromide (1.47 g, 4.71 mmol) in 2-(2-methoxyethoxy)ethanol (1.00 mL) was stirred at 130° C. for 4 hrs under nitrogen atmosphere. On completion, the reaction mixture was concentrated under reduced pressure The crude product was purified by reversed-phase HPLC (0.1% FA condition) to give the title compound (500 mg, 56% yield, FA) as a yellow oil. LC-MS (ESI⁺) m/z 229.1 (M+H)⁺; ¹H NMR (400 MHz, DMSO-d₆) δ=6.85-6.51 (m, 2H), 3.81 (s, 3H), 3.16 (s, 1H), 3.10 (d, J=3.2 Hz, 4H), 3.00 (d, J=4.8 Hz, 4H).

### 2-Bromopyridine-4-carbonitrile (CAS #10386-27-3) (Intermediate GK)

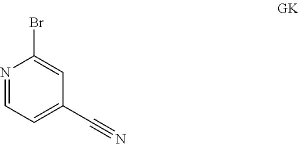

### 1-Ethyl-5-iodo-1H-pyrazole (CAS #1392274-29-1) (Intermediate GL)

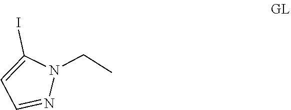

### 5-Fluoro-6-methoxy-1′,2′,3′,6′-tetrahydro-2,4′-bipyridine (Intermediate GM)

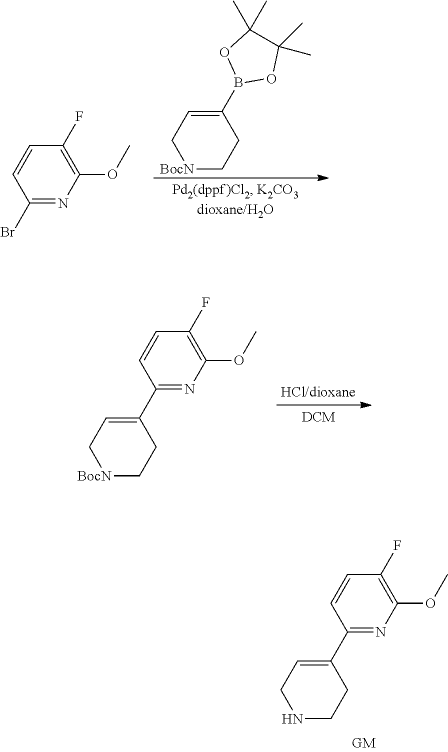

### Step 1—Tert-butyl 5-fluoro-6-methoxy-5′,6′-dihydro-[2,4′-bipyridine]-1′(2′H)-carboxylate

To a solution of 6-bromo-3-fluoro-2-methoxy-pyridine (199 mg, 970 μmol, CAS #1211529-85-9), and tert-butyl 4-(4,4,5,5-tetramethyl-1,3,2-dioxaborolan-2-yl)-3,6-dihydro-2H-pyridine-1-carboxylate (250 mg, 808 μmol, CAS #286961-14-6) in dioxane (2 mL) and H₂O (0.5 mL) was added Pd(dppf)Cl₂·CH₂Cl₂ (66.0 mg, 80.8 μmol) and K₂CO₃ (335. mg, 2.43 mmol). The mixture was stirred at 80° C. for 2 hrs under N₂ atmosphere. On completion, the reaction mixture was filtered and concentrated under reduced pressure to give a residue. The residue was purified by column chromatography (SiO₂, Petroleum ether/Ethyl acetate=50/1 to 20/1) to afford the title compound (210 mg, 82% yield) as a colorless oil. LC-MS (ESI⁺) m/z 253.0 (M+H)⁺.

### Step 2—5-Fluoro-6-methoxy-1′,2′,3′,6′-tetrahydro-2,4′-bipyridine

To a solution of tert-butyl 4-(5-fluoro-6-methoxy-2-pyridyl)-3,6-dihydro-2H-pyridine-1-carboxylate (210 mg, 681 μmol) in DCM (2 mL) was added HCl/dioxane (2 M, 2 mL). The mixture was then stirred at 25° C. for 1 hr. On completion, the reaction mixture was concentrated under reduced pressure to give the title compound (160 mg, HCl) as a yellow solid. LC-MS (ESI⁺) m/z 209.0 (M+H)⁺.

### 1-(3-Methoxy-5-(trifluoromethyl)pyridin-2-yl)piperazine (Intermediate GN)

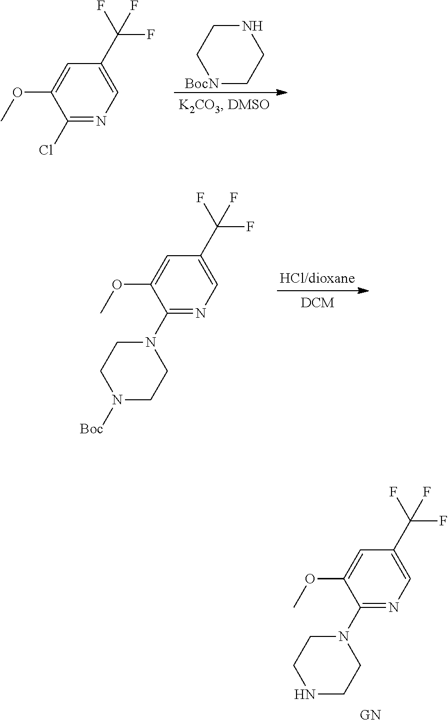

### Step 1—Tert-butyl 4-(3-methoxy-5-(trifluoromethyl)pyridin-2-yl)piperazine-1-carboxylate

To a solution of 6-chloro-5-methoxy-pyridine-3-carbonitrile (400 mg, 2.37 mmol, CAS #1256835-79-6) and tert-butyl piperazine-1-carboxylate (442 mg, 2.37 mmol, CAS #57260-71-6) in DMSO (10 mL) was added K₂CO₃ (984 mg, 7.12 mmol). The mixture was then stirred at 120° C. for 4 hrs. On completion, the reaction mixture was quenched by addition of H₂O (3 mL), and then extracted with EtOAc (20 mL×3). The combined organic layers were washed with brine (20 mL×3), dried over Na₂SO₄, filtered and concentrated under reduced pressure to give a residue. The crude residue was purified by column chromatography (SiO₂, Petroleum ether/Ethyl acetate=5/1) to give the title compound (700 mg, 93% yield) as a yellow solid. LC-MS (ESI⁺) m/z 218.9 (M−99)⁺.

### Step 2—1-(3-Methoxy-5-(trifluoromethyl)pyridin-2-yl)piperazine

A solution of tert-butyl 4-[3-methoxy-5-(trifluoromethyl)-2-pyridyl]piperazine-1-carboxylate (300 mg, 830 μmol) in DCM (4 mL) and HCl (1 mL) was stirred at 25° C. for 2 hrs. On completion, the mixture was concentrated in vacuo to give the title compound (230 mg) as a yellow solid. LC-MS (ESI⁺) m/z 262.1 (M+H)⁺.

### 5-Bromo-8-methoxy-2,3-dihydrobenzo[b][1,4]dioxine (Intermediate GO)

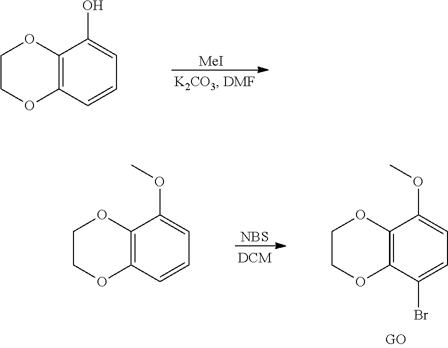

### Step 1—5-Methoxy-2,3-dihydrobenzo[b][1,4]dioxine

To a solution of 2,3-dihydrobenzo[b][1,4]dioxin-5-ol (1 g, 6.57 mmol, CAS #10288-36-65) and MeI (1.87 g, 13.1 mmol, 818 μL) in DMF (10 mL) was added K₂CO₃ (1.82 g, 13.1 mmol). The mixture was then stirred at 25° C. for 18 hrs. On completion, the reaction mixture was quenched with addition H₂O (20 mL), and then extracted with EA (3×30 mL). The combined organic layers were washed with aqueous NaCl (30 mL×3), dried over Na₂SO₄, filtered and concentrated under reduced pressure to give the title compound (0.8 g) as a white solid. LC-MS (ESI⁺) m/z 167.1 (M+H)⁺.

### Step 2—5-Bromo-8-methoxy-2,3-dihydrobenzo[b][1,4]dioxine

To a solution of 5-methoxy-2,3-dihydrobenzo[b][1,4]dioxine (0.8 g, 4.8 mmol) in DCM (10 mL) was added NBS (856 mg, 4.81 mmol). The mixture was stirred at 25° C. for 4 hrs. On completion, the reaction mixture was quenched with H₂O (2 mL) at 0° C., then filtered and the filtrate was concentrated in vacuo. The residue was purified by prep-HPLC (FA condition) to give the title compound (0.1 g, 7% yield, FA) as a white solid. LC-MS (ESI⁺) m/z 245.9 (M+H)⁺.

### N-methyl-2-(piperazin-1-yl)pyridin-3-amine (Intermediate GP)

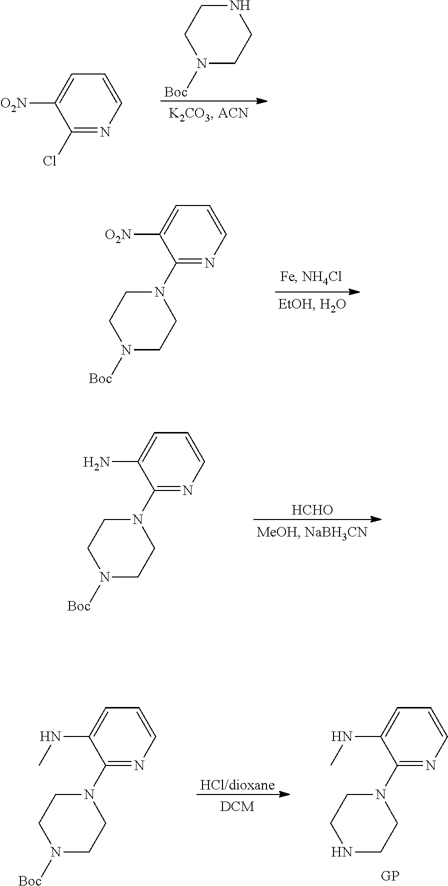

### Step 1—Tert-butyl 4-(3-nitropyridin-2-yl)piperazine-1-carboxylate

To a solution of 2-chloro-3-nitropyridine (3 g, 20 mmol, CAS #5470-18-8), and tert-butyl piperazine-1-carboxylate (3.52 g, 18.9 mmol, CAS #57260-71-6) in ACN (40 mL) was added K₂CO₃ (3.92 g, 28.38 mmol). Then the reaction was stirred at 25° C. for 12 hrs under nitrogen atmosphere. On completion, the reaction mixture was diluted with water (20 mL) and extracted with ethyl acetate (3×50 mL). The combined organic layers were washed with brine (20 mL) and dried over anhydrous sodium sulfate, filtered and the filtrate was concentrated in vacuo to give the title compound (5 g) as a yellow oil. LC-MS (ESI⁺) m/z 253.0 (M−55)⁺.

### Step 2—Tert-butyl 4-(3-aminopyridin-2-yl)piperazine-1-carboxylate

To a solution of tert-butyl 4-(3-nitropyridin-2-yl)piperazine-1-carboxylate (1 g, 3 mmol) in EtOH (6 mL) and H₂O (3 mL) was added Fe (1.81 g, 32.4 mmol) and NH₄Cl (1.73 g, 32.4 mmol). Then the reaction was stirred at 25° C. for 12 hrs. On completion, the reaction mixture was filtered and the filtrate was concentrated in vacuo to give the crude residue. The crude residue was purified by column chromatography (SiO₂, Petroleum ether:Ethyl acetate=10/1 to 1/1) to give the title compound (600 mg, 66% yield) as a yellow solid. LC-MS (ESI⁺) m/z 279.1 (M+H)⁺.

### Step 3—tert-butyl 4-(3-(methylamino)pyridin-2-yl)piperazine-1-carboxylate

To a solution of tert-butyl 4-(3-aminopyridin-2-yl)piperazine-1-carboxylate (550 mg, 1.98 mmol) and formaldehyde (801 mg, 9.88 mmol, 735 L, 37% solution, CAS #50-00-0) in MeOH (6 mL). After addition, the mixture was stirred at 25° C. for 1 hr, and then NaBH₃CN (310 mg, 4.94 mmol) was added. The resulting mixture was stirred at 25° C. for 1 hr. On completion, the reaction mixture was filtered and the filtrate was concentrated in vacuo to give the title compound (500 mg) as a yellow solid. LC-MS (ESI⁺) m/z 293.2 (M+H)⁺.

### Step 4—N-methyl-2-(piperazin-1-yl)pyridin-3-amine

To a solution of tert-butyl 4-(3-(methylamino)pyridin-2-yl)piperazine-1-carboxylate e (500 mg, 1.71 mmol) in DCM (5 mL) was added HCl/dioxane (8 M, 1 mL). The mixture was then stirred at 25° C. for 0.5 hrs. On completion, the reaction mixture was concentrated in vacuo to give the crude residue. The crude residue was purified by reversed-phase HPLC (0.8 g/L ammonium bicarbonate) to give the title compound (90 mg, 24% yield) as a white solid. LC-MS (ESI⁺) m/z 193.2 (M+H)⁺.

### Methyl 4-bromo-6-chloro-7-fluoro-1-((2-(trimethylsilyl)ethoxy)methyl)-1H-indole-2-carboxylate (Intermediate GO)

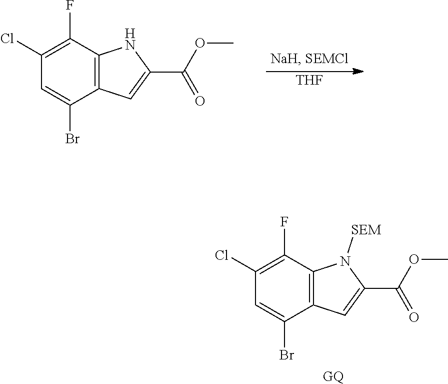 [SMILES: COC(=O)c1cc2c(Br)cc(Cl)c(F)c2[nH]1 ; COC(=O)c1cc2c(Br)cc(Cl)c(F)c2n1COCC[Si](C)(C)C ; "GQ" ; "THF"]

To a solution of methyl 4-bromo-6-chloro-7-fluoro-1H-indole-2-carboxylate (2 g, 7 mmol, synthesized via Steps 1-5 of Intermediate C) in THF (20 mL) was added NaH (521 mg, 13.0 mmol, 60% dispersion in mineral oil) at 0° C. under N₂ atmosphere. After stirring for 1 hr, SEM-Cl (1.09 g, 6.52 mmol, 1.15 mL) was added into the mixture. The reaction was stirred at 0° C. for 1 hr under N₂ atmosphere. On completion, the reaction mixture was quenched with NH₄Cl (20 mL) and extracted with ethyl acetate (3×50 mL). The combined organic layers were washed with brine (100 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 5/1) to give the title compound (2.2 g, 69% yield) as a white solid. ¹H NMR (400 MHz, DMSO-d6) δ=7.82 (d, J=5.6 Hz, 1H), 7.40 (d, J=1.6 Hz, 1H), 6.16 (s, 2H), 4.05 (s, 3H), 3.61 (t, J=7.6 Hz, 2H), 0.92 (t, J=7.6 Hz, 2H), 0.00 (s, 9H).

### 6-Chloro-7-fluoro-4-(1-methylpyrrolidin-3-yl)-1H-indole-2-carboxylic acid (Intermediate GR)

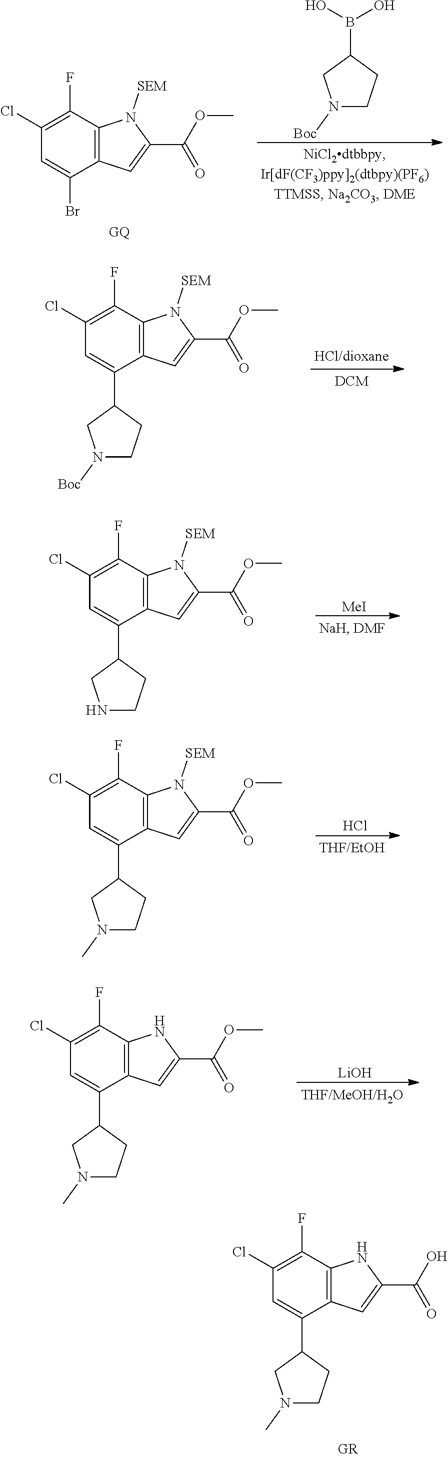

### Step 1—Methyl 4-(1-(tert-butoxycarbonyl)pyrrolidin-3-yl)-6-chloro-7-fluoro-1-((2-(trimethylsilyl)ethoxy)methyl)-1H-indole-2-carboxylate

A mixture of methyl 4-bromo-6-chloro-7-fluoro-1-((2-(trimethylsilyl)ethoxy)methyl)-1H-indole-2-carboxylate (0.5 g, 1.1 mmol, Intermediate GQ), (1-(tert-butoxycarbonyl)pyrrolidin-3-yl)boronic acid (369 mg, 1.72 mmol), Ir[dF(CF3)ppy]2[d(m-CF3)bpy](PF6) (65.5 mg, 57 μmol), NiCl₂·dtbpy (45.5 mg, 114 μmol) and morpholine (299 mg, 3.43 mmol, 302 μL) in DMF (5 mL) was degassed and purged with N₂ three times. Then the mixture was stirred at 25° C. for 4 hrs under N₂ atmosphere. On completion, the reaction mixture was filtered and concentrated under reduced pressure and purified by column chromatography (SiO₂, Petroleum ether/Ethyl acetate=1/0 to 10/1) to give the title compound (00 mg, 52% yield) as a white solid. LC-MS (ESI⁺) m/z 549.3 (M+Na)⁺.

### Step 2—Methyl 6-chloro-7-fluoro-4-(pyrrolidin-3-yl)-1-((2-(trimethylsilyl)ethoxy)methyl)-1H-indole-2-carboxylate

To a solution of methyl 4-(1-(tert-butoxycarbonyl)pyrrolidin-3-yl)-6-chloro-7-fluoro-1-((2-(trimethylsilyl)ethoxy)methyl)-1H-indole-2-carboxylate (400 mg, 758 μmol) in DCM (4 mL) was added HCl/dioxane (4 M, 189 μL). The mixture was then stirred at 25° C. for 2 hrs. On completion, the reaction mixture was filtered and the filtrate was concentrated in vacuo to give the title compound (320 mg, HCl) as a red solid. LC-MS (ESI⁺) m/z 427.2 (M+H)⁺.

### Step 3—Methyl 6-chloro-7-fluoro-4-(1-methylpyrrolidin-3-yl)-1-((2-(trimethylsilyl)ethoxy)methyl)-1H-indole-2-carboxylate

To a solution of formaldehyde (218 mg, 2.70 mmol, 200 μL) in MeOH (2 mL) was added methyl 6-chloro-7-fluoro-4-(pyrrolidin-3-yl)-1-((2-(trimethylsilyl)ethoxy)methyl)-1H-indole-2-carboxylate (250 mg, 539 μmol, HCl) and NaBH₃CN (67.8 mg, 1.08 mmol). The mixture was then stirred at 25° C. for 2 hrs. On completion, the reaction mixture was concentrated under reduced pressure to give a residue to give the title compound (200 mg) as a white solid. LC-MS (ESI⁺) m/z 441.1 (M+H)⁺.

### Step 4—Methyl 6-chloro-7-fluoro-4-(1-methylpyrrolidin-3-yl)-1H-indole-2-carboxylate

To a solution of methyl 6-chloro-7-fluoro-4-(1-methylpyrrolidin-3-yl)-1-((2-(trimethylsilyl)ethoxy)methyl)-1H-indole-2-carboxylate (200 mg, 453 μmol) in THF (1 mL) and EtOH (3 mL) was added HCl (6 M, 1 mL). The mixture was then stirred at 80° C. for 12 hrs. On completion, the reaction mixture was filtered and the filtrate was concentrated in vacuo to give the crude residue. The crude residue was purified prep-HPLC (FA condition) to give the title compound (90 mg, 31% yield, FA) as a white solid. LC-MS (ESI⁺) m/z 311.0 (M+1)⁺.

### Step 5—6-Chloro-7-fluoro-4-(1-methylpyrrolidin-3-yl)-1H-indole-2-carboxylic acid

To a solution of methyl 6-chloro-7-fluoro-4-(1-methylpyrrolidin-3-yl)-1H-indole-2-carboxylate (90 mg, 289 μmol) in THE (2 mL), MeOH (0.5 mL) and H₂O (0.5 mL) was added LiOH·H₂O (24.3 mg, 579 μmol). The mixture was then stirred at 25° C. for 12 hrs. On completion, the reaction mixture was concentrated in vacuo and purified by prep-HPLC (FA condition) to give the title compound (90 mg, 88% yield, FA) as a white solid. LC-MS (ESI⁺) m/z 297.1 (M+1)⁺.

### (6-Chloro-7-fluoro-4-(1-methylpyrrolidin-3-yl)-1H-indol-2-yl)(4-(3-methoxypyridin-2-yl)piperazin-1-yl)methanone (Intermediate GS)

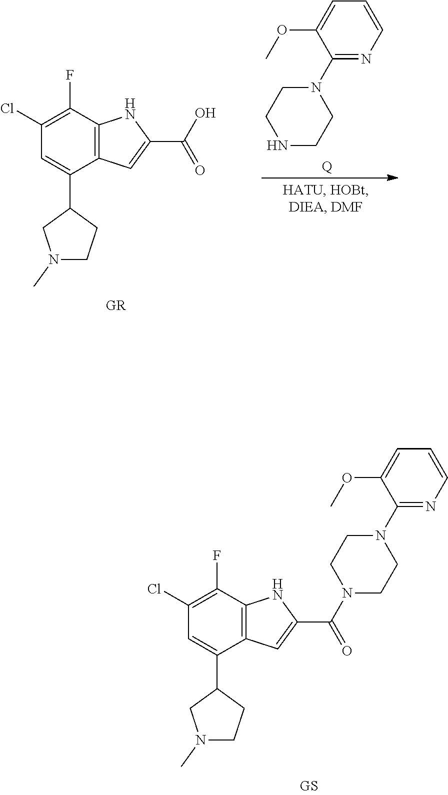

To a solution of 6-chloro-7-fluoro-4-(1-methylpyrrolidin-3-yl)-1H-indole-2-carboxylic acid (50 mg, 168 μmol, Intermediate GR) in DMF (2 mL) was added HATU (76 mg, 202 μmol) and HOBt (34 mg, 252 μmol), DIEA (108 mg, 842 μmol, 146 μL) and 1-(3-methoxypyridin-2-yl)piperazine (46.4 mg, 202 μmol, HCl, Intermediate Q). The mixture was then stirred at 25° C. for 2 hrs. On completion, the reaction mixture was triturated with H₂O at 0° C. for 15 min, then filtered and dried to give the title compound (50 mg) as a white solid. LC-MS (ESI⁺) m/z 472.2 (M+H)⁺.

### (6-Chloro-4-(3,5-dimethyl-1H-pyrazol-1-yl)-7-fluoro-1H-indol-2-yl)(4-(3-methoxypyridin-2-yl)piperazin-1-yl)methanone (Intermediate GT)

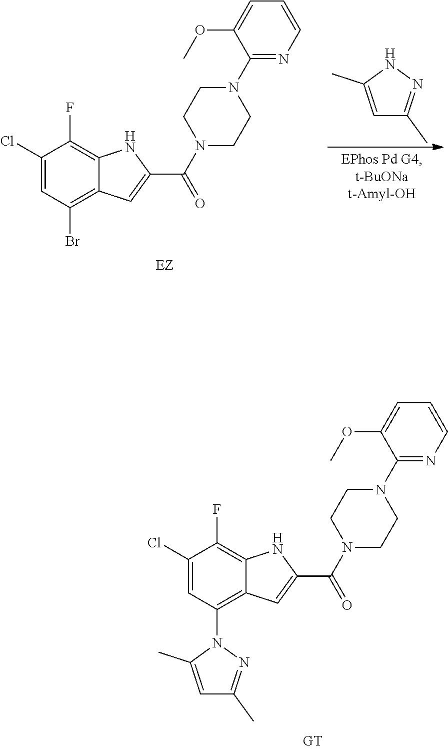

A mixture of (4-bromo-6-chloro-7-fluoro-1H-indol-2-yl)(4-(3-methoxypyridin-2-yl)piperazin-1-yl)methanone (200 mg, 427 μmol, Intermediate EZ), 3,5-dimethyl-1H-pyrazole (49.3 mg, 513 μmol, CAS #67-51-6), EPhos Pd G4 (20.0 mg, 42. μmol), and tBuONa (2 M, 641 μL) in t-Amyl-OH (10 mL) was degassed and purged with N₂ three times. Then the mixture was stirred at 90° C. for 2 hrs under N₂ atmosphere. On completion, the crude product was triturated with water at 25° C. for 0.5 hours, then the mixture was filtered and the filter cake was dried in vacuo to give the title compound (200 mg) as a yellow solid. LC-MS (ESI⁺) m/z 483.0 (M+H)⁺.

### 1-(6-Methoxypyridin-2-yl)piperazine (Intermediate GU)

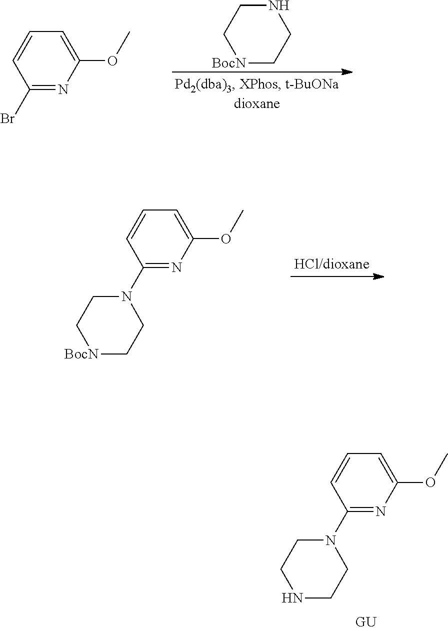

### Step 1—Tert-butyl 4-(6-methoxypyridin-2-yl)piperazine-1-carboxylate

A mixture of 2-bromo-6-methoxy-pyridine (1.00 g, 5.32 mmol, 654 μL, CAS #40473-07-2), tert-butyl piperazine-1-carboxylate (991 mg, 5.32 mmol, CAS #57260-71-6), Pd₂(dba)₃ (487 mg, 532 μmol), XPhos (254 mg, 532 μmol) and t-BuONa (1.53 g, 16.0 mmol) in dioxane (10.0 mL) was degassed and purged with N₂ three times. Then the mixture was stirred at 80° C. for 12 hrs under N₂ atmosphere. On completion, the reaction mixture was filtered and concentrated under reduced pressure to give a residue. The crude product was purified by MPLC (SiO₂, PE:EA=50:1 to 20:1) to give the title compound (1.30 g, 77% yield) as a yellow solid. LC-MS (ESI⁺) m/z 238.1 (M−56+H)⁺; ¹H NMR (400 MHz, CHLOROFORM-d) δ=7.42 (t, J=8.0 Hz, 1H), 6.16 (d, J=8.0 Hz, 1H), 6.11 (d, J=8.0 Hz, 1H), 3.87 (s, 3H), 3.53 (s, 4H), 3.52 (s, 4H), 1.49 (s, 9H).

### Step 2—1-(6-Methoxypyridin-2-yl)piperazine

To a solution of tert-butyl 4-(6-methoxy-2-pyridyl)piperazine-1-carboxylate (200 mg, 682 μmol) in DCM (2.00 mL) and was added HCl/dioxane (8 M, 1 mL), then the mixture was stirred at 25° C. for 0.5 hr. On completion, the reaction mixture was filtered and concentrated under reduced pressure to give the title compound (150 mg, HCl) as a white solid. LC-MS (ESI⁺) m/z 194.1 (M+H)⁺.

### 5-Fluoro-3-methoxy-1′,2′,3′,6′-tetrahydro-2,4′-bipyridine (Intermediate GV)

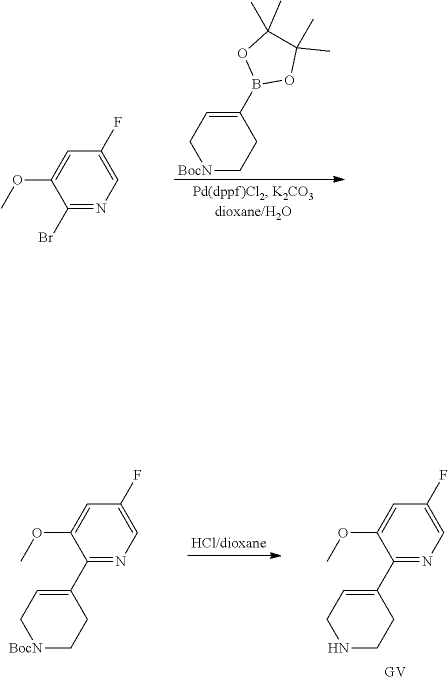

### Step 1—Tert-butyl 5-fluoro-3-methoxy-3′,6′-dihydro-[2,4′-bipyridine]-1′(2′H)-carboxylate

To a solution of 2-bromo-5-fluoro-3-methoxy-pyridine (0.5 g, 2.4 mmol) tert-butyl 4-(4,4,5,5-tetramethyl-1,3,2-dioxaborolan-2-yl)-3,6-dihydro-2H-pyridine-1-carboxylate (901 mg, 2.91 mmol) in dioxane (10 mL) and H₂O (2.5 mL) was added K₂CO₃ (1.01 g, 7.28 mmol) and Pd(dppf)Cl₂·CH₂Cl₂ (198 mg, 243 μmol). The mixture was then stirred at 80° C. for 12 hrs. On completion, the reaction mixture was quenched by addition of H₂O (20 mL), and then diluted with EA (20 mL) and extracted with EA (100 mL×2). The combined organic layers were washed with NaCl (50 mL×2), dried over Na₂SO₄, filtered and concentrated under reduced pressure to give a residue. The residue was purified by column chromatography (SiO₂, Petroleum ether/Ethyl acetate=20/1 to 6/1) to give the title compound (0.7 g, 84% yield) as a white solid. LC-MS (ESI⁺) m/z 253.1 (M+H)⁺.

### Step 2—5-Fluoro-3-methoxy-1′,2′,3′,6′-tetrahydro-2,4′-bipyridine

To a solution of tert-butyl 4-(5-fluoro-3-methoxy-2-pyridyl)-3,6-dihydro-2H-pyridine-1-carboxylate (300 mg, 973 μmol) in DCM (0.1 mL) was added HCl/dioxane (2 M, 3 mL). The mixture was then stirred at 25° C. for 1 hr. On completion, the reaction mixture was filtered and concentrated under reduced pressure to give the title compound (200 mg, HCl) as a white solid. LC-MS (ESI⁺) m/z 180.2 (M+H)⁺.

### 6-Methoxy-1′,2′,3′,6′-tetrahydro-2,4′-bipyridine (Intermediate GW)

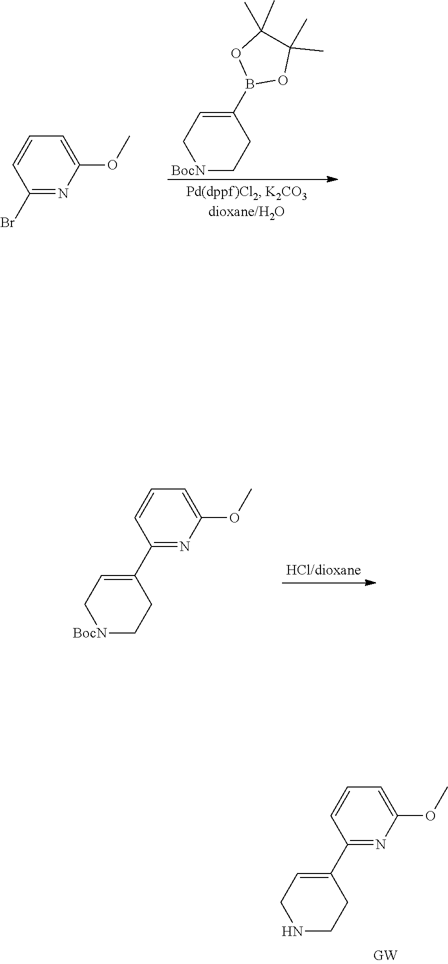

### Step 1—Tert-butyl 6-methoxy-3′,6′-dihydro-[2,4′-bipyridine]-1′(2′H)-carboxylate

A mixture of 2-bromo-6-methoxy-pyridine (3 g, 16 mmol, 1.96 mL), tert-butyl 4-(4,4,5,5-tetramethyl-1,3,2-dioxaborolan-2-yl)-3,6-dihydro-2H-pyridine-1-carboxylate (5.92 g, 19.2 mmol), Pd(dppf)Cl₂·CH₂Cl₂ (1.30 g, 1.60 mmol), and K₂CO₃ (6.62 g, 47.9 mmol) in dioxane (40 mL) and H₂O (10 mL) was degassed and purged with N₂ three times. Then the mixture was stirred at 80° C. for 12 hr under N₂ atmosphere. On completion, the reaction mixture was quenched with H₂O (20 mL), and then diluted with EA (20 mL) and extracted with EA (100 mL×2). The combined organic layers were washed with NaCl (50 mL×2), dried over Na₂SO₄, filtered and concentrated under reduced pressure to give a residue. The residue was purified by column chromatography (SiO₂, Petroleum ether/Ethyl acetate=30/1 to 10/1) to give the title compound (4.5 g, 97% yield) as a white solid. LC-MS (ESI⁺) m/z 291.1 (M+H)⁺.

### Step 2—6-Methoxy-1′,2′,3′,6′-tetrahydro-2,4′-bipyridine

To a solution of tert-butyl 4-(6-methoxy-2-pyridyl)-3,6-dihydro-2H-pyridine-1-carboxylate (1 g, 3 mmol) in DCM (0.5 mL) was added HCl/dioxane (2 M, 10 mL). The mixture was then stirred at 25° C. for 1 hr. On completion, the reaction mixture was filtered and concentrated under reduced pressure to give the title compound (600 mg) as a white solid. LC-MS (ESI⁺) m/z 162.2 (M+H)⁺.

### 1-(5-Fluoro-6-methoxy-2-pyridyl)piperazine (Intermediate GX)

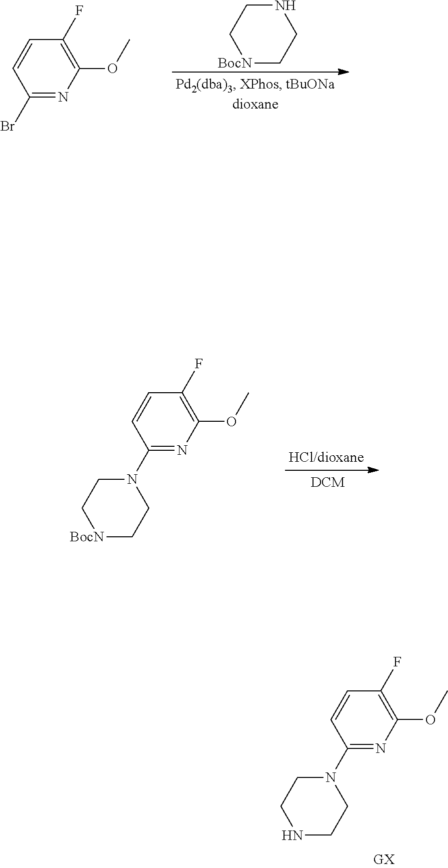

### Step 1—Tert-butyl 4-(5-fluoro-6-methoxypyridin-2-yl)piperazine-1-carboxylate

A mixture of 6-bromo-3-fluoro-2-methoxy-pyridine (531 mg, 2.58 mmol), tert-butyl piperazine-1-carboxylate (400 mg, 2.15 mmol), XPhos (102 mg, 215 μmol), tBuONa (620 mg, 6.44 mmol) and Pd₂(dba)₃ (197 mg, 215 μmol) in dioxane (5.00 mL) was degassed and purged with N₂ three times. Then the mixture was stirred at 100° C. for 2 hrs under N₂ atmosphere. On completion, the reaction mixture was filtered and concentrated under reduced pressure to give a residue. The crude product was purified by MPLC (SiO₂, PE:EA=50:1 to 20:1) to give the title compound (580 mg, 78% yield) as a yellow oil. LC-MS (ESI⁺) m/z 256.0 (M−55)⁺.

### Step 2—1-(5-Fluoro-6-methoxypyridin-2-yl)piperazine

To a solution of tert-butyl 4-(5-fluoro-6-methoxy-2-pyridyl)piperazine-1-carboxylate (580 mg, 1.86 mmol) in DCM (5.00 mL) was added HCl/dioxane (2 M, 5 mL). The mixture was then stirred at 25° C. for 1 hr. On completion, the reaction mixture was concentrated under reduced pressure to give a residue. The crude product was triturated with DCM at 25° C. for 10 mins to give the title compound (460 mg, 100% yield, HCl) as a pink solid. LC-MS (ESI⁺) m/z 212.0 (M+H)⁺.

### 6-(1-(3-(1H-1,2,3-triazol-1-yl)propanoyl)-1,2,5,6-tetrahydropyridin-3-yl)-7-fluoro-4-(4-methoxyquinolin-3-yl)-1H-indole-2-carboxylic acid (Intermediate GY)

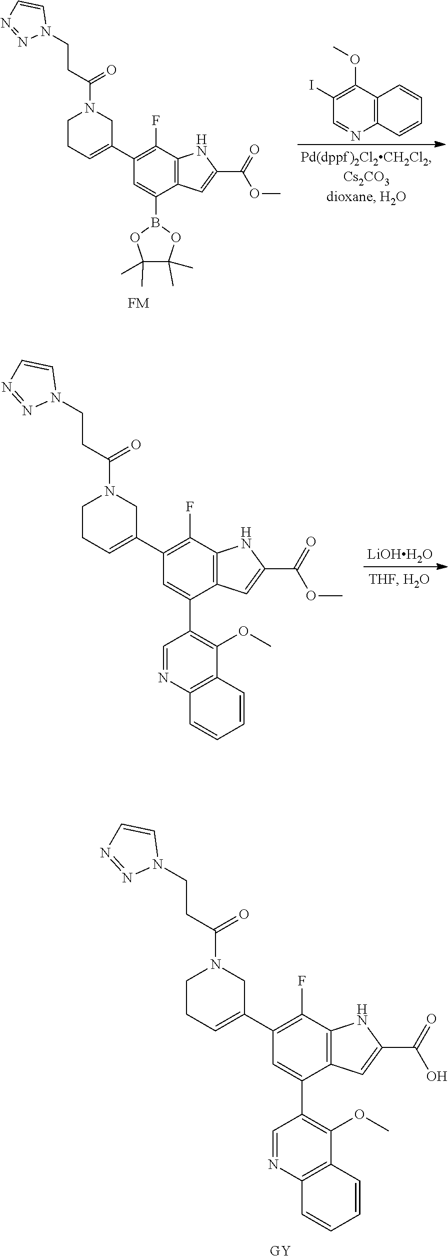

### Step 1—Methyl 6-(1-(3-(1H-1,2,3-triazol-1-yl)propanoyl)-1,2,5,6-tetrahydropyridin-3-yl)-7-fluoro-4-(4-methoxyquinolin-3-yl)-1H-indole-2-carboxylate

A mixture of methyl 6-(1-(3-(1H-1,2,3-triazol-1-yl)propanoyl)-1,2,5,6-tetrahydropyridin-3-yl)-7-fluoro-4-(4,4,5,5-tetramethyl-1,3,2-dioxaborolan-2-yl)-1H-indole-2-carboxylate (1.00 g, 1.91 mmol, Intermediate FM), 3-iodo-4-methoxyquinoline (653 mg, 2.29 mmol, CAS #1260836-74-5), Cs₂CO₃ (1.87 g, 5.73 mmol), and Pd(dppf)Cl₂·CH₂Cl₂ (156 mg, 191 μmol) in dioxane (10 mL) was degassed and purged with N₂ three times. Then the mixture was stirred at 80° C. for 2 hrs under N₂ atmosphere. On completion, the crude product was triturated with water at 25° C. for 1 hr, then the mixture was filtered and the filter cake was dried in vacuo to give the title compound. LC-MS (ESI⁺) m/z 555.2 (M+H)⁺.

### Step 2—6-(1-(3-(1H-1,2,3-triazol-1-yl)propanoyl)-1,2,5,6-tetrahydropyridin-3-yl)-7-fluoro-4-(4-methoxyquinolin-3-yl)-1H-indole-2-carboxylic acid

A mixture of methyl 6-(1-(3-(1H-1,2,3-triazol-1-yl)propanoyl)-1,2,5,6-tetrahydropyridin-3-yl)-7-fluoro-4-(4-methoxyquinolin-3-yl)-1H-indole-2-carboxylate (600 mg, 1.08 mmol) and LiOH·H₂O (227 mg, 5.41 mmol) in THF (6 mL) and H₂O (6 mL) was stirred at 40° C. for 2 hrs. On completion, to the reaction mixture was added HCl (1N) until the pH 5, then the mixture was diluted with water (20 mL). The mixture was concentrated in vacuo to give the compound (0.3 g) as a yellow solid. LC-MS (ESI⁺) m/z 541.1 (M+H)⁺.

### 1-(5-(4-Chloro-2-(4-cyclopropylpiperazine-1-carbonyl)-7-fluoro-1H-indol-6-yl)-3,6-dihydropyridin-1(2H)-yl)-3-(1H-1,2,3-triazol-1-yl)propan-1-one (Intermediate GZ)

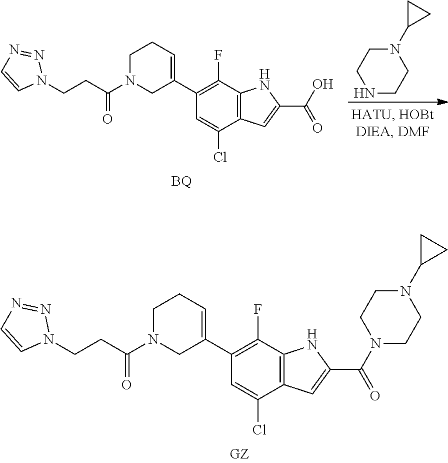 [SMILES: O=C(O)c1cc2c(Cl)cc(C3=CCCN(C(=O)CCn4ccnn4)C3)c(F)c2[nH]1 ; C1CN(C2CC2)CCN1 ; O=C(CCn1ccnn1)N1CCC=C(c2cc(Cl)c3cc(C(=O)N4CCN(C5CC5)CC4)[nH]c3c2F)C1]

To a solution of 4-chloro-7-fluoro-6-[1-[3-(triazol-1-yl)propanoyl]-3,6-dihydro-2H-pyridin-5-yl]-1H-indole-2-carboxylic acid (600 mg, 1.44 mmol, Intermediate BQ) in DMF (6 mL) was added HATU (709 mg, 1.87 mmol), DIEA (742 mg, 5.74 mmol, 1.00 mL), HOBt (232 mg, 1.72 mmol) and 1-cyclopropylpiperazine (235 mg, 1.87 mmol, CAS #20327-23-5). Then the reaction was stirred at 25° C. for 10 min. On completion, the reaction mixture was diluted with water (10 mL) and extracted with ethyl acetate (3×30 mL). The combined organic layers were washed with brine (10 mL) and dried over anhydrous sodium sulfate, filtered and the filtrate was concentrated in vacuo to give the crude residue. The crude residue was purified by column chromatography (SiO₂, Dichloromethane. Methanol=1/0 to 10/1) to give the title compound (320 mg, 29% yield) as a yellow solid. LC-MS (ESI⁺) m/z 526.2 (M+H)⁺.

### (4-Fluoro-2-methoxyphenyl)boronic acid CAS #179899-07-1) (Intermediate HA)

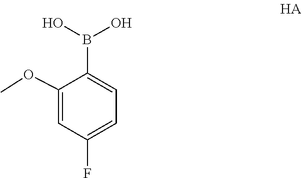

### (3,5-Difluoro-2-methoxyphenyl)boronic acid (Intermediate HB)

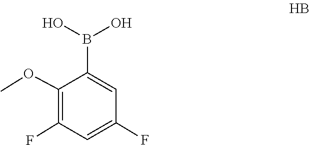

### 1-(5-(4-chloro-7-fluoro-2-(4-(6-methoxypyridin-2-yl)piperazine-1-carbonyl)-1H-indol-6-yl)-3,6-dihydropyridin-1(2H)-yl)-3-(1H-1,2,3-triazol-1-yl)propan-1-one (Intermediate HC)

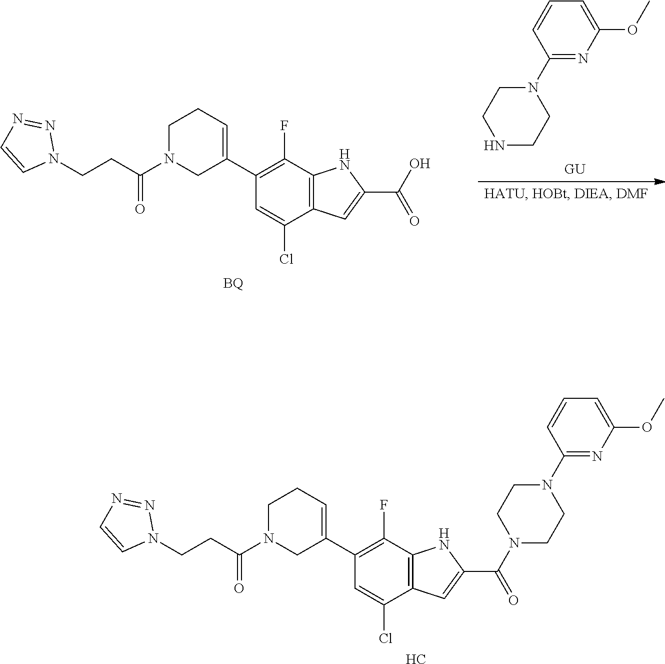

To a solution of 4-chloro-7-fluoro-6-[1-[3-(triazol-1-yl)propanoyl]-3,6-dihydro-2H-pyridin-5-yl]-1H-indole-2-carboxylic acid (1.6 g, 3.8 μmol, Intermediate BQ) in DMF (20 mL) was added HATU (1.60 g, 4.21 μmol), DIEA (1.98 g, 15.3 μmol) and HOBt (1.03 g, 7.66 μmol). Then 1-(6-methoxy-2-pyridyl)piperazine (1.14 g, 4.98 μmol, HCl, Intermediate GU) was added and the mixture was stirred at 0-25° C. for 20 minus. On completion, the reaction mixture was added to H₂O (100 mL) at 25° C., and then filtered and concentrated under reduced pressure to give a residue. The crude product was triturated with PE: EA=1:1, filtered and dried under reduced pressure to give the title compound (1.4 g, 46% yield) as a yellow solid. LC-MS (ESI⁺) m/z 593.2 (M+H)⁺.

### 1-(4-Chloro-2-methoxyphenyl)piperazine (Intermediate HD)

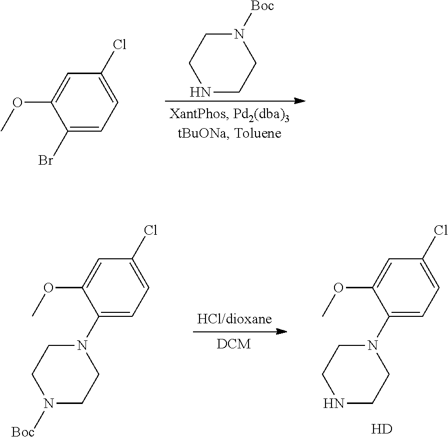

### Step 1—Tert-butyl 4-(4-chloro-2-methoxyphenyl)piperazine-1-carboxylate

A mixture of 1-bromo-4-chloro-2-methoxybenzene (5 g, 20 mmol, CAS #194913-74-8), tert-butyl piperazine-1-carboxylate (6.31 g, 33.8 mmol, CAS #57260-71-6), Pd₂(dba)₃ (1.03 g, 1.13 mmol), tBuONa (6.51 g, 67.7 mmol) and Xantphos (1.31 g, 2.26 mmol) in dioxane (60 mL) was degassed and purged with N₂ three times. Then the mixture was stirred at 100° C. for 4 hrs under N₂ atmosphere. On completion, the reaction mixture was concentrated under reduced pressure and purified by column chromatography (SiO₂, Petroleum ether/Ethyl acetate=1/0 to 2/1) to give the title compound (2.67 g, 31% yield) as a yellow solid. LC-MS (ESI⁺) m/z 327.1 (M+H)⁺.

### Step 2—1-(4-Chloro-2-methoxyphenyl)piperazine

To a solution of tert-butyl 4-(4-chloro-2-methoxyphenyl)piperazine-1-carboxylate (2 g, 6 mmol in DCM (20 mL) was added HCl/dioxane (4 M, 1.53 mL). The mixture was then stirred at 25° C. for 2 hrs. On completion, the reaction mixture was filtered and concentrated under reduced pressure to give the title compound (1.3 g, HCl) as a white solid. LC-MS (ESI⁺) m/z 227.0 (M+H)⁺.

### (6-Chloro-7-fluoro-4-(1,4-oxazepan-4-yl)-1H-indol-2-yl)(4-(3-methoxypyridin-2-yl)piperazin-1-yl)methanone (Intermediate HE)

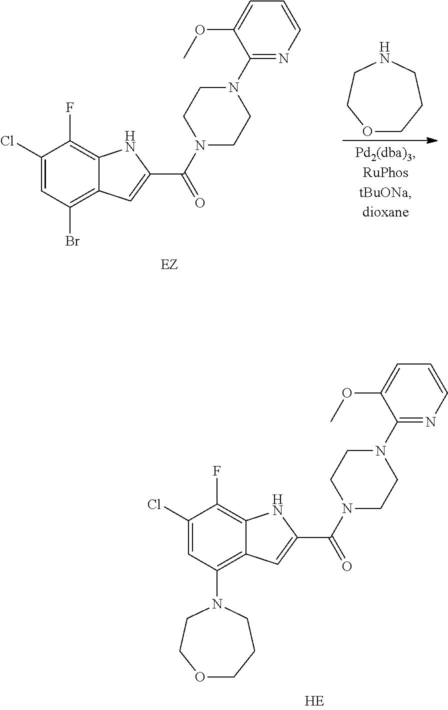 [SMILES: COc1cccnc1N1CCN(C(=O)c2cc3c(Br)cc(Cl)c(F)c3[nH]2)CC1 ; C1CNCCOC1 ; COc1cccnc1N1CCN(C(=O)c2cc3c(N4CCCOCC4)cc(Cl)c(F)c3[nH]2)CC1]

To a solution of (4-bromo-6-chloro-7-fluoro-1H-indol-2-yl)(4-(3-methoxypyridin-2-yl)piperazin-1-yl)methanone (150 mg, 320 μmol, Intermediate EZ), 1,4-oxazepane (42.1 mg, 416 μmol, CAS #5638-60-8) in dioxane (2 mL) was added tBuONa (92.4 mg, 962 μmol), RuPhos (14.9 mg, 32.1 μmol), and Pd₂(dba)₃ (29.3 mg, 32.1 μmol). Then the reaction was stirred at 80° C. for 1 hr under nitrogen atmosphere. On completion, the reaction mixture was diluted with water (10 mL) and extracted with ethyl acetate (3×30 mL). The combined organic layers were washed with brine (10 mL) and dried over anhydrous sodium sulfate, filtered and the filtrate was concentrated in vacuo to give the crude residue. The crude residue was purified by column chromatography (SiO₂, Dichloromethane. Methanol=1/0 to 10/1) to give the title compound (90 mg, 51% yield) as a yellow solid. LC-MS (ESI⁺) m/z 488.2 (M+H)⁺.

### 1-(5-Ethyl-3-methoxypyridin-2-yl)piperazine (Intermediate HF)

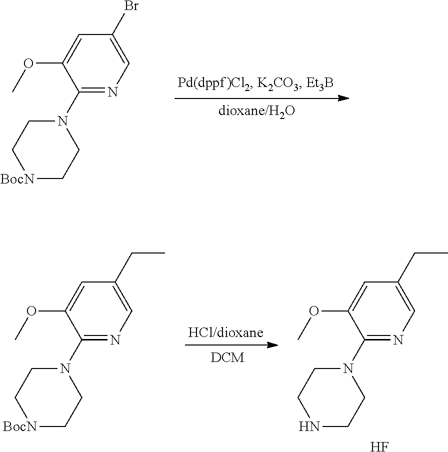

### Step 1—Tert-butyl 4-(5-ethyl-3-methoxypyridin-2-yl)piperazine-1-carboxylate

To a solution of tert-butyl 4-(5-bromo-3-methoxy-2-pyridyl)piperazine-1-carboxylate (500 mg, 1.34 mmol, synthesized via Step 1 of Intermediate AY) and triethylborane (1 M, 1.34 mL, CAS #97-94-9) in dioxane (5 mL) and H₂O (1 mL) was added Pd(dppf)Cl₂ (98.2 mg, 134 μmol) and K₂CO₃ (556 mg, 4.03 mmol). The mixture was then stirred at 80° C. for 2 hrs under N₂ atmosphere. On completion, the reaction mixture was filtered and concentrated under reduced pressure to give a residue. The residue was purified by column chromatography (SiO₂, Petroleum ether/Ethyl acetate=50/1 to 10/1) to give the title compound (300 mg, 65% yield) as a yellow solid. LC-MS (ESI⁺) m/z 322.2 (M+H)⁺.

### Step 2—1-(5-Ethyl-3-methoxypyridin-2-yl)piperazine

To a solution of tert-butyl 4-(5-ethyl-3-methoxy-2-pyridyl)piperazine-1-carboxylate (300 mg, 933 μmol) in DCM (3 mL) was added HCl/dioxane (2 M, 3 mL). Then the mixture was stirred at 25° C. for 1 hr. On completion, the reaction mixture was concentrated under reduced pressure to give the title compound (200 mg, HCl) as a white solid. LC-MS (ESI⁺) m/z 222.2 (M+H)⁺.

### 1-(5-Isopropyl-3-methoxypyridin-2-yl)piperazine (Intermediate HG)

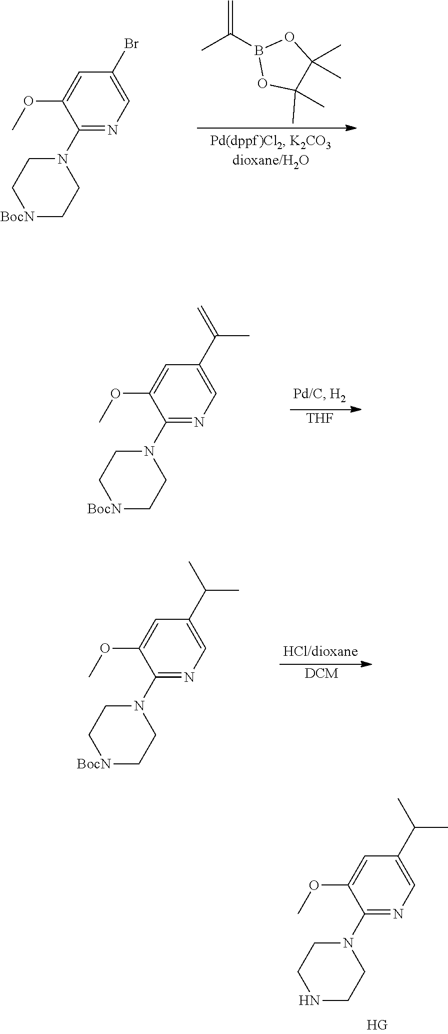

### Step 1—Tert-butyl 4-(3-methoxy-5-(prop-1-en-2-yl)pyridin-2-yl)piperazine-1-carboxylate

To a solution of tert-butyl 4-(5-bromo-3-methoxy-2-pyridyl)piperazine-1-carboxylate (500 mg, 1.34 mmol, synthesized via Step 1 of Intermediate AY) and 2-isopropenyl-4,4,5,5-tetramethyl-1,3,2-dioxaborolane (270 mg, 1.61 mmol, CAS #126726-62-3) in dioxane (5 mL) and H₂O (1 mL) was added Pd(dppf)Cl₂ (98.3 mg, 134 μmol) and K₂CO₃ (557 mg, 4.03 mmol). The mixture was then stirred at 85° C. for 1 hr under N₂ atmosphere. On completion, the reaction mixture was filtered and concentrated under reduced pressure to give a residue. The residue was purified by column chromatography (SiO₂, Petroleum ether/Ethyl acetate=20/1 to 7/1) to afford the title compound (320 mg, 71% yield) as a yellow solid. LC-MS (ESI⁺) m/z 334.2 (M+H)⁺.

### Step 2—Tert-butyl 4-(5-isopropyl-3-methoxypyridin-2-yl)piperazine-1-carboxylate

To a solution of tert-butyl 4-(5-isopropenyl-3-methoxy-2-pyridyl)piperazine-1-carboxylate (320 mg, 959 μmol) in THF (3 mL) was added Pd/C (300 mg, 281 μmol, 10 wt %) under N₂ atmosphere. The mixture was then stirred at 25° C. for 12 hrs under H₂ atmosphere, 15 psi. On completion, the reaction mixture was filtered and concentrated under reduced pressure to give the title compound (320 mg, 99% yield) as a white solid. LC-MS (ESI⁺) m/z 336.2 (M+H)⁺.

### Step 3—1-(5-Isopropyl-3-methoxypyridin-2-yl)piperazine

To a solution of tert-butyl 4-(5-isopropyl-3-methoxy-2-pyridyl)piperazine-1-carboxylate (320 mg, 954 μmol) in DCM (3 mL) was added HCl/dioxane (2 M, 3 mL). The mixture was then stirred at 25° C. for 2 hrs. On completion, the reaction mixture was filtered and concentrated under reduced pressure to give the title compound (250 mg, HCl) as a white solid. LC-MS (ESI⁺) m/z 236.2 (M+H)⁺.

### 1-(3,5-Dimethoxy-6-methylpyridin-2-yl)piperazine (Intermediate HH)

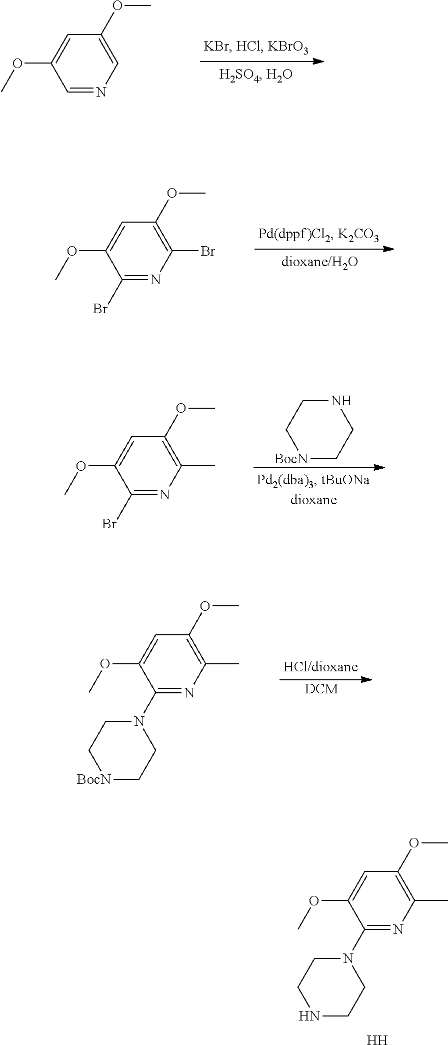

### Step 1—2,6-Dibromo-3,5-dimethoxypyridine

3,5-dimethoxypyridine (500 mg, 3.59 mmol, CAS #18677-48-0) and HCl (1 M, 4.70 mL) was added to a 40 ml vial. Then KBr (2.73 g, 22.9 mmol) and H₂SO₄ (3 M, 9.40 mL) was added to the vial. The mixture was then added to a 100 ml flask contained KBrO₃ (752 mg, 4.50 mmol) and H₂O (30 mL). Then the mixture was stirred at 25° C. for 10 mins. On completion, Na₂SO₃ aq (1.3 M, 50 mL) was added and stirred for 2 min, then the mixture was extracted with DCM (20 mL×5). The combined organic layers were washed with NaOH (1M, 50 mL), dried over Na₂SO₄, filtered and concentrated under reduced pressure to give the title compound (940 mg, 88% yield) as a white solid. LC-MS (ESI⁺) m/z 295.8 (M+H)⁺; ¹H NMR (400 MHz, DMSO-d6) δ=7.28 (s, 1H), 3.96 (s, 611).

### Step 2—2-Bromo-3,5-dimethoxy-6-methylpyridine

To a solution of methylboronic acid (181 mg, 3.03 mmol, CAS #13061-96-6) and 2,6-dibromo-3,5-dimethoxy-pyridine (900 mg, 3.03 mmol) in dioxane (10 mL) and H₂O (2 mL) was added Pd(dppf)Cl₂ (221 mg, 303 μmol) and K₂CO₃ (1.26 g, 9.09 mmol). Then the mixture was stirred at 80° C. for 2 hrs under N₂ atmosphere. On completion, the reaction mixture was filtered and concentrated under reduced pressure to give a residue. The residue was purified by column chromatography (SiO₂, Petroleum ether/Ethyl acetate=20/1 to 5/1) to give the title compound (130 mg, 13% yield) as a white solid. LC-MS (ESI⁺) m/z 231.9 (M+H)⁺.

### Step 3—Tert-butyl 4-(3,5-dimethoxy-6-methylpyridin-2-yl)piperazine-1-carboxylate

To a solution of tert-butyl piperazine-1-carboxylate (52.2 mg, 280 μmol, CAS #57260-71-6) and 2-bromo-3,5-dimethoxy-6-methyl-pyridine (130 mg, 560 μmol) in dioxane (1 mL) was added Pd₂(dba)₃ (25.6 mg, 28.0 μmol) and tBuONa (134 mg, 1.40 mmol). The mixture was then stirred at 100° C. for 12 hrs under N₂ atmosphere. On completion, the reaction mixture was quenched by addition of NH₄Cl (5 mL) and extracted with DCM (20 mL×3). The combined organic layers were washed with brine (20 mL×2), dried over Na₂SO₄, filtered and concentrated under reduced pressure to give a residue. The residue was purified by column chromatography (SiO₂, DCM:MeOH=10:1) give the title compound (50 mg, 26% yield) as a yellow solid. LC-MS (ESI⁺) m/z 338.2 (M+H)⁺.

### Step 4—1-(3,5-Dimethoxy-6-methylpyridin-2-yl)piperazine

To a solution of tert-butyl 4-(3,5-dimethoxy-6-methyl-2-pyridyl)piperazine-1-carboxylate (30 mg, 88.9 μmol) in DCM (0.5 mL) was added HCl/dioxane (2 M, 300 μL). The mixture was then stirred at 25° C. for 2 hrs. On completion, the reaction mixture was filtered and concentrated under reduced pressure to give the title compound (25 mg, 92% yield, HCl) as a white solid. LC-MS (ESI⁺) m/z 238.2 (M+H)⁺.

### 1-[5-[4-Chloro-2-[4-[3-(cyclopropoxy)-2-pyridyl]piperazine-1-carbonyl]-7-fluoro-1H-indol-6-yl]-3,6-dihydro-2H-pyridin-1-yl]-3-(triazol-1-yl)propan-1-one (Intermediate HI)

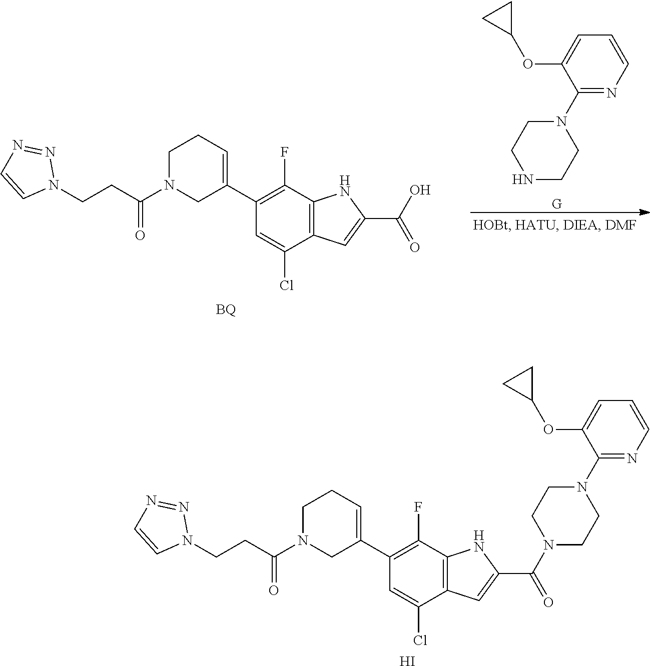

To a solution of 4-chloro-7-fluoro-6-[1-[3-(triazol-1-yl)propanoyl]-3,6-dihydro-2H-pyridin-5-yl]-1H-indole-2-carboxylic acid (1.2 g, 2.9 mmol, Intermediate BQ) 1-[3-(cyclopropoxy)-2-pyridyl]piperazine (693 mg, 3.16 mmol, Intermediate G) in DMF (15 mL) was added HOBt (504 mg, 3.73 mmol), HATU (1.42 g, 3.73 mmol) and DIEA (1.86 g, 14.4 mmol, 2.50 mL). Then the mixture was stirred at 25° C. for 0.5 hr. On completion, the reaction mixture was quenched by addition of H₂O (10 mL), and then diluted with EA (20 mL) and extracted with EA (100 mL×2). The combined organic layers were washed with NaCl (30 mL×2), dried over Na₂SO₄, filtered and concentrated under reduced pressure to give a residue. The crude product was purified by reversed-phase HPLC(0.1% FA condition) to give the title compound (800 mg, 40% yield) as a white solid. LC-MS (ESI⁺) m/z 619.3 (M+H)⁺.

### 6-(1-(3-(1H-1,2,3-triazol-1-yl)propanoyl)-1,2,5,6-tetrahydropyridin-3-yl)-7-fluoro-4-(4-fluoro-2-methoxyphenyl)-1H-indole-2-carboxylic acid (Intermediate HJ), 6-(1-(3-(1H-1,2,3-triazol-1-yl)propanoyl)-1,2,5,6-tetrahydropyridin-3-yl)-4-(4,5-difluoro-2-methoxyphenyl)-7-fluoro-1H-indole-2-carboxylic acid (Intermediate HK) and 6-(1-(3-(1H-1,2,3-triazol-1-yl)propanoyl)-1,2,5,6-tetrahydropyridin-3-yl)-4-(3,5-difluoro-2-methoxyphenyl)-7-fluoro-1H-indole-2-carboxylic acid (Intermediate HL)

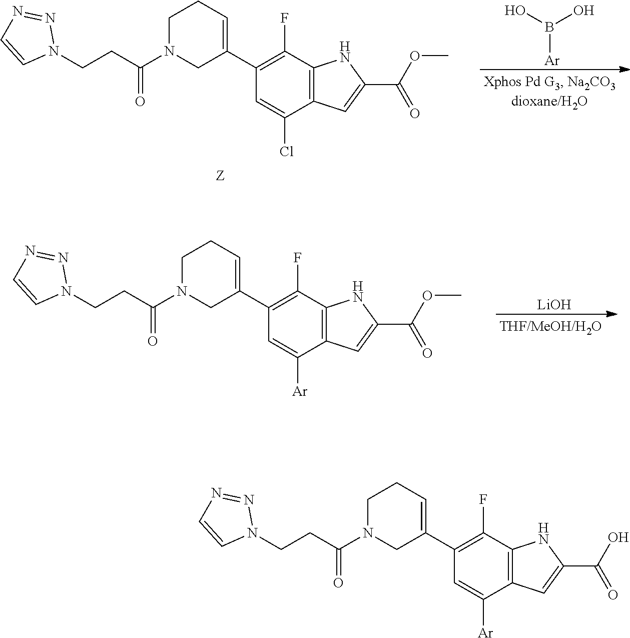

Ar:

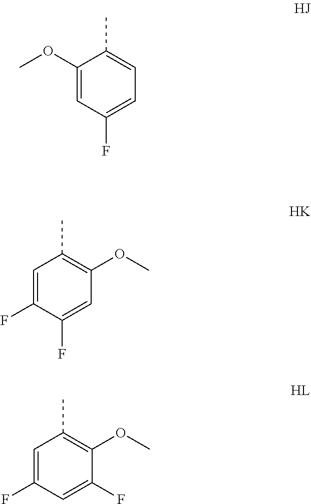

### Step 1

To a solution of methyl 4-chloro-7-fluoro-6-[1-[3-(triazol-1-yl)propanoyl]-3,6-dihydro-2H-pyridin-5-yl]-1H-indole-2-carboxylate (500 mg, 1 eq, Intermediate Z) and Aryl boronic acid (1.2 eq) in dioxane (8 mL) and H₂O (2 mL) was added Na₂CO₃ (3 eq) and XPhos Pd G3 (0.1 eq). Then the mixture was stirred at 80° C. for 4 h. On completion, the reaction mixture was concentrated under reduced pressure to remove solvent. The residue was diluted with H₂O (10 mL) and extracted with EA (20 mL×3). The combined organic layers were washed with NaCl (20 mL×2), dried over Na₂SO₄, filtered and the filtrate was concentrated under reduced pressure to give a residue. The reaction mixture was filtered and concentrated under reduced pressure to give a residue. The residue was purified by column chromatography to give the title compounds.

### Step 2

To a solution of methyl 4-(3,5-difluoro-2-methoxy-phenyl)-7-fluoro-6-[1-[3-(triazol-1-yl)propanoyl]-3,6-dihydro-2H-pyridin-5-yl]-1H-indole-2-carboxylate (1 eq) in THF (4 mL), MeOH (1 mL) and H₂O (1 mL) was added LiOH·H2O (5 eq). The mixture was then stirred at 40° C. for 1 h. On completion, the reaction mixture was concentrated under reduced pressure to remove solvent. The residue was diluted with H₂O (5 mL) and extracted with EA (10 mL×3). The combined organic layers were washed with NaCl (10 mL×2), dried over Na₂SO₄, filtered and concentrated under reduced pressure to give the title compounds.

### 6-(1-(3-(1H-1,2,3-triazol-1-yl)propanoyl)-1,2,5,6-tetrahydropyridin-3-yl)-7-fluoro-4-(2-methoxypyridin-3-yl)-1H-indole-2-carboxylic acid (Intermediate HM)

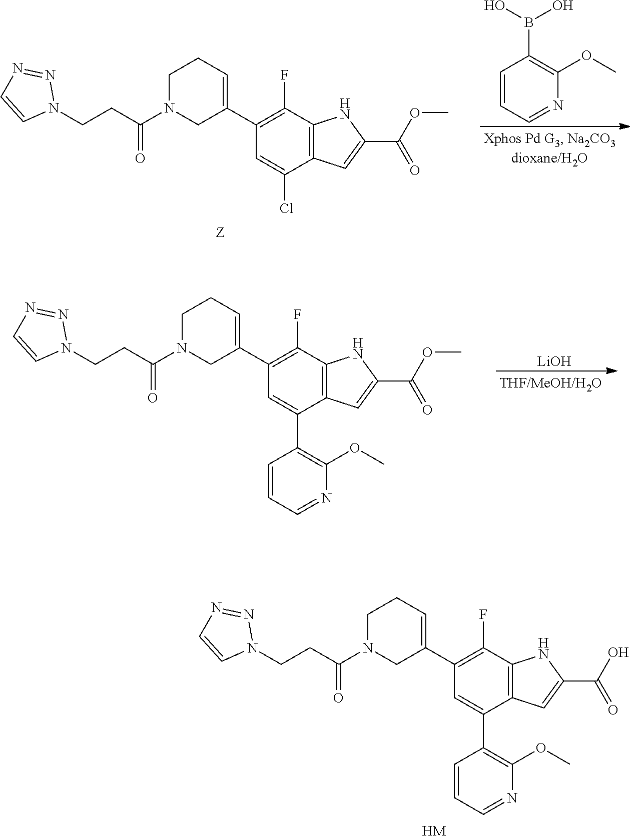

### Step 1—Methyl 6-(1-(3-(1H-1,2,3-triazol-1-yl)propanoyl)-1,2,5,6-tetrahydropyridin-3-yl)-7-fluoro-4-(2-methoxypyridin-3-yl)-1H-indole-2-carboxylate

To a solution of (2-methoxypyridin-3-yl)boronic acid (212 mg, 1.39 mmol, CAS #163105-90-6) and methyl 6-(1-(3-(1H-1,2,3-triazol-1-yl)propanoyl)-1,2,5,6-tetrahydropyridin-3-yl)-4-chloro-7-fluoro-1H-indole-2-carboxylate (500 mg, 1.16 mmol, Intermediate Z) in dioxane (5 mL) and H₂O (1 mL) was added Na₂CO₃ (368 mg, 3.47 mmol) and XPhos Pd G3 (98.0 mg, 115 μmol) at 25° C. under nitrogen atmosphere. Then the mixture was stirred at 80° C. for 1 hr under nitrogen atmosphere. On completion, the reaction mixture was concentrated in vacuo to give the crude residue. The residue was purified by column chromatography (SiO₂, Petroleum ether/Ethyl acetate=0/1 to 5/1, DCM:MeOH=20:1) to give the title compound (380 mg, 57% yield) as a white solid. LC-MS (ESI⁺) m/z 505.2 (M+H)⁺.

### Step 2—6-(1-(3-(1H-1,2,3-triazol-1-yl)propanoyl)-1,2,5,6-tetrahydropyridin-3-yl)-7-fluoro-4-(2-methoxypyridin-3-yl)-1H-indole-2-carboxylic acid

To a solution of methyl 6-(1-(3-(1H-1,2,3-triazol-1-yl)propanoyl)-1,2,5,6-tetrahydropyridin-3-yl)-7-fluoro-4-(2-methoxypyridin-3-yl)-1H-indole-2-carboxylate (380 mg, 753 μmol) in anhydrous THF (4 mL), H₂O (1 mL) and MeOH (1 mL) was added LiOH·H₂O (158 mg, 3.77 mmol) at 25° C. Then the reaction was stirred at 40° C. for 2 hrs. On completion, the mixture was extracted with dichloromethane (2×5 mL). Then the aqueous phase was added HCl (1N) until pH 1, then extracted with ethyl acetate (3×10 mL), The combined organic layers were dried over anhydrous sodium sulfate, filtered and the filtrate was concentrated in vacuo to give the title compound (250 mg, HCl) as a white solid. LC-MS (ESI⁺) m/z 491.2 (M+H)⁺.

### 1-(2-Methoxy-4-(trifluoromethyl)phenyl)piperazine (CAS #1121600-45-0) (Intermediate HN)

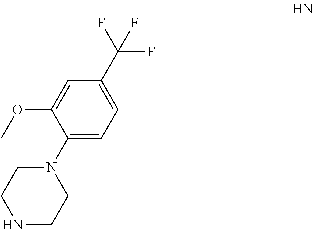 [SMILES: COc1cc(C(F)(F)F)ccc1N1CCNCC1]

### 3-Bromo-4-isopropylpyridine (Intermediate HO)

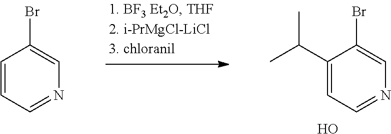

To a solution of 3-bromopyridine (2.5 g, 16 mmol, CAS #626-55-1) in THF (25 mL) was added BF₃·Et₂O (2.47 g, 17.4 mmol) at 0° C. and stirred for 15 mins. Then i-PrMgCl—LiCl (1.3 M, 14.6 mL) was added at −50° C. and the mixture was stirred for 30 minutes. Finally chloranil (7.78 g, 31.7 mmol) was added and then the mixture was warmed up to 25° C. The mixture was then stirred at 25° C. for 2 hrs. On completion, the reaction mixture was quenched with NH₃H₂O (12 ml) and diluted with water (100 mL) and extracted with dichloromethane (3×50 mL). The combined organic layers were washed with brine (2×50 mL) and dried over anhydrous sodium sulfate, filtered and the filtrate was concentrated in vacuo to give the crude residue. The residue was purified by column chromatography (SiO₂, Petroleum ether:Ethyl acetate=20/0 to 5/1) to give the title compound (1.7 g, 48% yield) as a red oil. LC-MS (ESI⁺) m/z 202.0 (M+H)⁺.

### 4-Chloro-3-ethylpyridine (Intermediate HP)

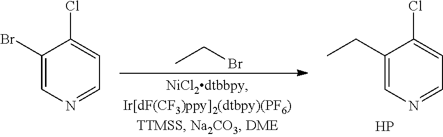

A mixture of 3-bromo-4-chloropyridine (1 g, 5 mmol), bromoethane (566 mg, 5.2 mmol), bis[3,5-difluoro-2-[5-(trifluoromethyl)-2-pyridyl]phenyl]iridium(1+);4-tert-butyl-2-(4-tert-butyl-2-pyridyl)pyridine hexafluorophosphate (58.3 mg, 52 μmol), 4-tert-butyl-2-(4-tert-butyl-2-pyridyl)pyridine;dichloronickel (31.0 mg, 78 μmol), bis(trimethylsilyl)silyl-trimethyl-silane (1.29 g, 5.2 mmol) and disodium carbonate (1.1 g, 10.4 mmol) in DME (20 mL) was degassed and purged with N₂ three times. Then the mixture was stirred at 25° C. for 2 hrs under N₂ atmosphere. The mixture was filtered and concentrated under reduced pressure. The residue was purified by silica gel column chromatography (petroleum ether/ethyl acetate=50/1˜20/1) to give the title compound (182 mg, 7% yield) as a yellow solid. LC-MS (ESI⁺) m/z 141.8 (M+H)⁺.

### 1-(4-Methoxypyridin-3-yl)piperazine (Intermediate HQ)

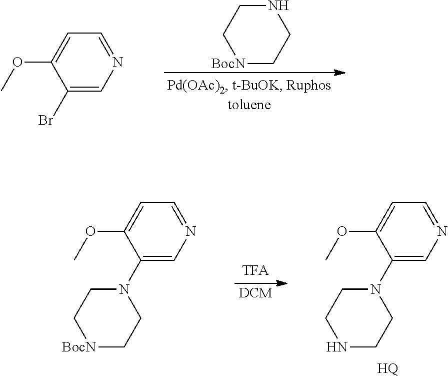

### Step 1—Tert-butyl 4-(4-methoxypyridin-3-yl)piperazine-1-carboxylate

To a solution of 3-bromo-4-methoxy-pyridine (1 g, 5 mmol, CAS #82257-09-8) in toluene (10 mL) was added RuPhos (496 mg, 1 mmol), tert-butyl piperazine-1-carboxylate (1.3 g, 6.9 mmol), Pd(OAc)₂ (119 mg, 531 μmol) and t-BuOK (1.79 g, 15.9 mmol). The mixture was then stirred at 90° C. for 12 hrs. On completion, the reaction mixture was concentrated under reduced pressure to remove toluene. The residue was purified by prep-HPLC (FA condition) to give a residue to give the title compound (170 mg, 7% yield, FA) as a white solid. LC-MS (ESI⁺) m/z 294.0 (M+H)⁺.

### Step 2—1-(4-Methoxypyridin-3-yl)piperazine

To a solution of tert-butyl 4-(4-methoxy-3-pyridyl)piperazine-1-carboxylate (150 mg, 511 mol) in DCM (3 mL) was added TFA (1.54 g, 13.4 mmol, 1 mL). The mixture was then stirred at 25° C. for 0.5 hr. On completion, the reaction mixture was concentrated under reduced pressure to remove DCM to give the title compound (98 mg, TFA) as a white solid. LC-MS (ESI⁺) m/z 193.1 (M+H)⁺.

### Tert-butyl 4-(6-bromo-3-methoxypyridin-2-yl)piperazine-1-carboxylate (Intermediate HR)

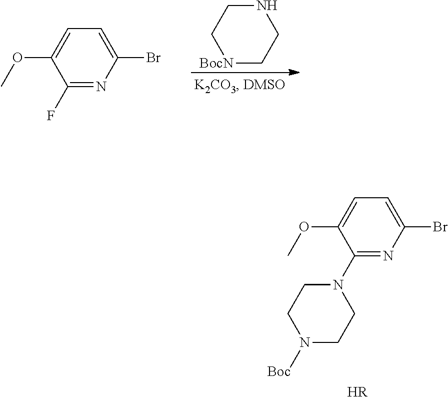

To a solution of 6-bromo-2-fluoro-3-methoxy-pyridine (1 g, 5 mmol, CAS #850142-73-3) in DMSO (10 mL) was added K₂CO₃ (2.01 g, 14.5 mmol) and tert-butyl piperazine-1-carboxylate (904 mg, 4.85 mmol, CAS #57260-71-6). The mixture was then stirred at 80° C. for 12 hrs. On completion, the reaction mixture was diluted with H₂O (20 mL) and extracted with EA (15 mL×3). The combined organic layers were washed with aqueous NaCl (20 mL×2), dried over Na₂SO₄, filtered and concentrated under reduced pressure to give a residue. The residue was purified by column chromatography (SiO₂, Petroleum ether/Ethyl acetate=50/1 to 10/1) to afford the title compound (1.67 g, 77% yield) as a yellow solid. LC-MS (ESI⁺) m/z 372.1 (M+H)⁺; ¹H NMR (400 MHz, DMSO-d₆) δ=7.22 (d, J=8.4 Hz, 1H), 7.04 (d, J=8.0 Hz, 1H), 3.79 (s, 3H), 3.41 (s, 4H), 3.28 (d, J=5.2 Hz, 4H), 1.41 (s, 9H).

### 1-(6-Ethyl-3-methoxypyridin-2-yl)piperazine (Intermediate HS)

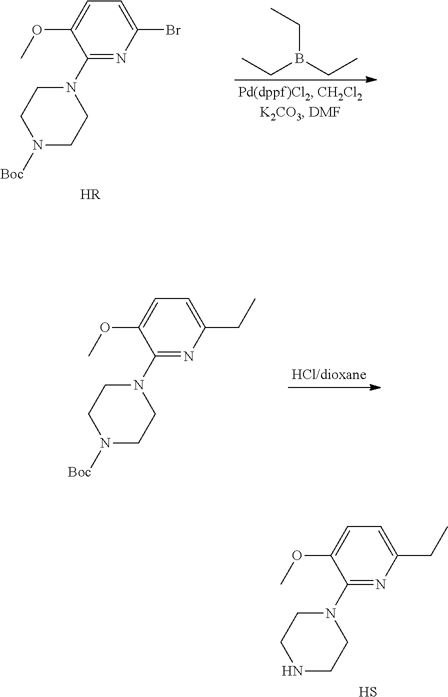

### Step 1—Tert-butyl 4-(6-ethyl-3-methoxypyridin-2-yl)piperazine-1-carboxylate

A mixture of tert-butyl 4-(6-bromo-3-methoxy-2-pyridyl)piperazine-1-carboxylate (500 mg, 1.34 mmol, Intermediate HR), triethylborane (1 M, 4.03 mL, CAS #97-94-9), Pd(dppf)Cl₂·CH₂Cl₂ (219 mg, 268 μmol) and K₂CO₃ (556 mg, 4.03 mmol) in DMF (10 mL) was degassed and purged with N₂ three times. Then the mixture was stirred at 65° C. for 2 hrs under N₂ atmosphere. On completion, the reaction mixture was quenched by addition of H₂O (30 mL) at 25° C., and then extracted with EA (20 mL×3). The combined organic layers were washed with aqueous NaCl (20 mL×2), dried over Na₂SO₄, filtered and concentrated under reduced pressure to give a residue. The residue was purified by column chromatography (SiO₂, Petroleum ether/Ethyl acetate=50/1 to 4/1) to afford the title compound (400 mg, 91% yield) as a colorless oil. LC-MS (ESI⁺) m/z 322.5 (M+H)⁺; ¹H NMR (400 MHz, CDCl₃-d) δ=7.04-6.92 (m, 1H), 6.68 (d, J=8.0 Hz, 1H), 3.83 (s, 3H), 3.60-3.55 (m, 4H), 3.37 (s, 4H), 2.65 (d, J=6.8 Hz, 2H), 1.49 (s, 9H), 1.25 (t, J=7.6 Hz, 3H).

### Step 2—1-(6-Ethyl-3-methoxypyridin-2-yl)piperazine

To a solution of tert-butyl 4-(6-ethyl-3-methoxy-2-pyridyl)piperazine-1-carboxylate (450 mg, 1.40 mmol) in HCl/dioxane (2 M, 4.50 mL). The mixture was then stirred at 25° C. for 1 hr. On completion, the reaction mixture was concentrated under reduced pressure to afford the title compound (360 mg, HCl) as a white solid. LC-MS (ESI⁺) m/z 222.4 (M+H)⁺.

### 4-Methoxy-3-(piperazin-1-yl)pyridazine (Intermediate HT)

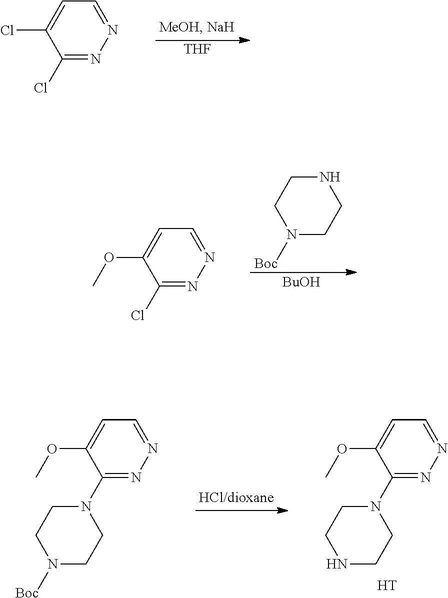

### Step 1—3-Chloro-4-methoxypyridazine

To a solution of NaH (280 mg, 7 mmol, 60% dispersion in mineral oil) in MeOH (5 mL) and THF (10 mL) was added 3,4-dichloropyridazine (1 g, 7 mmol, CAS #1677-80-1) at 0° C. The mixture was stirred at 0-25° C. for 12 hrs. On completion, the reaction mixture was quenched by addition of NH₄Cl (20 mL) at 0° C., and then diluted with DCM (20 mL) and extracted with DCM (30 mL×2). The combined organic layers were washed with NaCl (20 mL×2), dried over Na₂SO₄, filtered and concentrated under reduced pressure to give a residue. The residue was purified by column chromatography (SiO₂, Petroleum ether/Ethyl acetate=50/1 to 10/1) to give the title compound (0.45 g, 39% yield) as a white solid. LC-MS (ESI+) m/z 145.0 (M+H)⁺; ¹H NMR (400 MHz, DMSO-d6) δ=9.05 (d, J=5.6 Hz, 1H), 7.44 (d, J=5.6 Hz, 1H), 3.99 (s, 3H).

### Step 2—Tert-butyl 4-(4-methoxypyridazin-3-yl)piperazine-1-carboxylate

To a solution of 3-chloro-4-methoxy-pyridazine (0.2 g, 1.4 mmol) tert-butyl piperazine-1-carboxylate (1.03 g, 5.53 mmol) in n-BuOH (8 mL). Then the mixture was stirred at 150° C. for 2 hrs. On completion, the reaction mixture was quenched by addition of H₂O (10 mL), and then diluted with EA (20 mL) and extracted with EA (30 mL×2). The combined organic layers were washed with NaCl (30 mL×2), dried over Na₂SO₄, filtered and concentrated under reduced pressure to give a residue. The crude product was purified by reversed-phase HPLC(0.1% NH₄HCO₃ condition) to give the title compound (65 mg, 15% yield) as a white solid. LC-MS (ESI+) m/z 295.1 (M+H)⁺.

### Step 3—4-Methoxy-3-(piperazin-1-yl)pyridazine

To a solution of tert-butyl 4-(4-methoxypyridazin-3-yl)piperazine-1-carboxylate (60 mg, 200 μmol) in DCM (0.1 mL) was added HCl/dioxane (2 M, 1 mL). The mixture was then stirred at 25° C. for 0.5 hr. On completion, the reaction mixture was filtered and concentrated under reduced pressure to give the title compound (40 mg) as a white solid. LC-MS (ESI+) m/z 195.3 (M+H)⁺.

### 4-Methoxy-5-(piperazin-1-yl)pyrimidine (Intermediate HU)

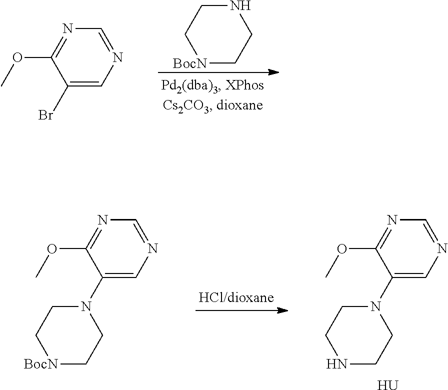

### Step 1—Tert-butyl 4-(4-methoxypyrimidin-5-yl)piperazine-1-carboxylate

A mixture of 5-bromo-4-methoxy-pyrimidine (800 mg, 4.23 mmol, CAS #4319-85-1), tert-butyl piperazine-1-carboxylate (788 mg, 4.23 mmol), Pd₂(dba)₃ (388 mg, 423 μmol), Xantphos (245 mg, 423 μmol) and Cs₂CO₃ (4.14 g, 12.7 mmol) in dioxane (5.0 mL) was degassed and purged with N₂ three times. Then the mixture was stirred at 100° C. for 12 hrs under N₂ atmosphere. The reaction mixture was filtered and concentrated under reduced pressure to give a residue. The crude product was purified by MPLC (SiO₂, PE:EA=10:1 to 5:1) to give the title compound (1.20 g, 75% yield) as a yellow oil. LC-MS (ESI+) m/z 295.2 (M+H)⁺; ¹H NMR (400 MHz, CHLOROFORM-d) δ=8.47 (s, 1H), 8.01 (s, 1H), 4.06 (s, 3H), 3.61 (d, J=4.0 Hz, 4H), 3.07 (s, 4H), 1.49 (d, J=0.8 Hz, 9H).

### Step 2—4-Methoxy-5-(piperazin-1-yl)pyrimidine

To a solution of tert-butyl 4-(4-methoxypyrimidin-5-yl)piperazine-1-carboxylate (400 mg, 1.36 mmol) in DCM (4.0 mL) and was added HCl/dioxane (8 M, 2 mL), then the mixture was stirred at 25° C. for 0.5 hr. On completion, the reaction mixture was filtered and concentrated under reduced pressure to give a residue to give the title compound (300 mg, HCl) as a white solid. LC-MS (ESI+) m/z 195.2 (M+H)⁺.

### 6-(1-(3-(1H-1,2,3-triazol-1-yl)propanoyl)-1,2,5,6-tetrahydropyridin-3-yl)-4-(4-ethoxypyridin-3-yl)-7-fluoro-1H-indole-2-carboxylic acid (Intermediate HV)

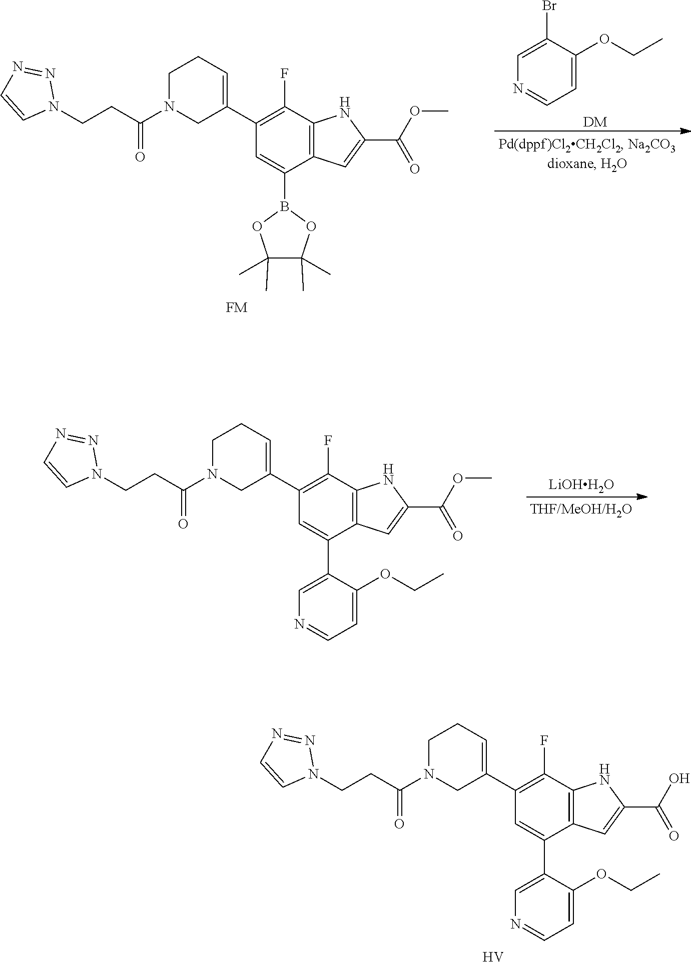

### Step 1—Methyl 6-(1-(3-(1H-1,2,3-triazol-1-yl)propanoyl)-1,2,5,6-tetrahydropyridin-3-yl)-4-(4-ethoxypyridin-3-yl)-7-fluoro-1H-indole-2-carboxylate

A mixture of 3-bromo-4-ethoxy-pyridine (540 mg, 2.68 mmol, Intermediate DM), methyl 7-fluoro-4-(4,4,5,5-tetramethyl-1,3,2-dioxaborolan-2-yl)-6-[1-[3-(triazol-1-yl)propanoyl]-3,6-dihydro-2H-pyridin-5-yl]-1H-indole-2-carboxylate (1.40 g, 2.68 mmol, Intermediate FM), Pd(dppf)Cl₂·CH₂Cl₂ (218 mg, 267 μmol), and Na₂CO₃ (851 mg, 8.03 mmol) in dioxane (10.0 mL) and H₂O (2.00 mL) was degassed and purged with N₂ three times. Then the mixture was stirred at 80° C. for 5 hrs under N₂ atmosphere. On completion, the reaction mixture was filtered and concentrated under reduced pressure to give a residue. The crude product was purified by MPLC(SiO₂, PE:EA:DCM=1:0:0 to 1:1:1) to give a residue to give the title compound (1.4 g, 72% yield) as a yellow oil. LC-MS (ESI+) m/z 519.2 (M+H)⁺; ¹H NMR (400 MHz, CHLOROFORM-d) δ=9.43-9.28 (m, 1H), 8.52 (d, J=10.8 Hz, 2H), 7.76 (d, J=2.4 Hz, 1H), 7.67 (s, 1H), 7.27 (s, 1H), 7.06 (s, 1H), 6.17 (d, J=15.2 Hz, 1H), 4.81-4.76 (m, 2H), 4.48 (s, 1H), 4.30 (s, 1H), 4.21-4.15 (m, 2H), 3.94 (s, 3H), 3.09-3.04 (m, 2H), 2.38 (s, 2H), 1.36-1.31 (m, 3H), 1.31-1.24 (m, 2H).

### Step 2—6-(1-(3-(1H-1,2,3-triazol-1-yl)propanoyl)-1,2,5,6-tetrahydropyridin-3-yl)-4-(4-ethoxypyridin-3-yl)-7-fluoro-1H-indole-2-carboxylic acid

To a solution of methyl 4-(4-ethoxy-3-pyridyl)-7-fluoro-6-[1-[3-(triazol-1-yl)propanoyl]-3,6-dihydro-2H-pyridin-5-yl]-1H-indole-2-carboxylate (1.40 g, 2.70 mmol) in MeOH (1.50 mL), THF (6.00 mL) and H₂O (1.50 mL) was added LiOH·H₂O (566 mg, 13.5 mmol). Then the mixture was stirred at 25° C. for 12 hrs. On completion, the reaction mixture was diluted with H₂O (10.0 mL) and HCl (5.00 mL), then was extracted with EA (50.0 mL×2). The combined organic layers were washed with sat. NaCl solution (50.0 mL×3), dried over Na₂SO₄, filtered and concentrated under reduced pressure to give a residue. The crude product was purified by reversed-phase HPLC(0.1% HCl condition) to give the title compound (420 mg, 28% yield, HCl) as a yellow solid. LC-MS (ESI+) m/z 505.1 (M+H)⁺.

### 3-Methoxy-N,N-dimethyl-4-(4,4,5,5-tetramethyl-1,3,2-dioxaborolan-2-yl)aniline (Intermediate HW)

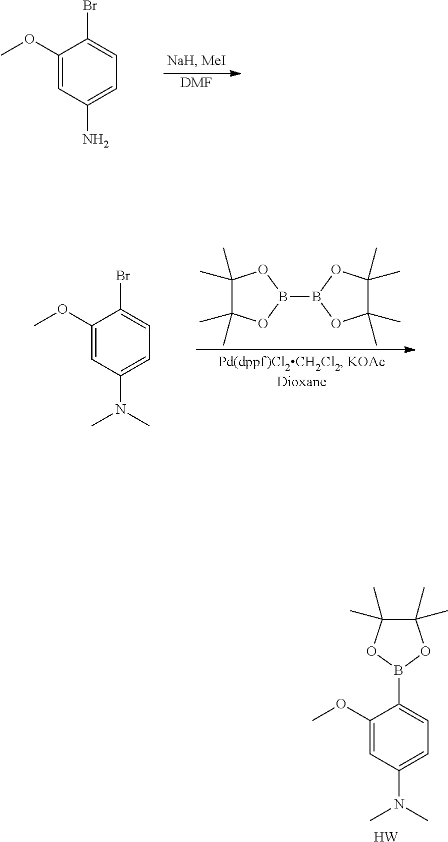

### Step 1—4-Bromo-3-methoxy-N,N-dimethylaniline

To a solution of 4-bromo-3-methoxyaniline (20 g, 100 mmol, CAS #19056-40-7) in DMF (200 mL) was added NaH (7.92 g, 198 mmol, 60% dispersion in mineral oil) under N₂ atmosphere at 0° C. The mixture was then stirred at 0° C. for 1 hr, and then MeI (28.1 g, 198 mmol, 12.3 mL) was added at 0° C. The resulting mixture was stirred at 25° C. for 12 hrs. On completion, the reaction mixture was quenched with ice water (200 mL) at 0° C. and extracted with ethyl acetate (3×100 mL). The combined organic layers were washed with brine (200 mL) and dried over anhydrous sodium sulfate, filtered and the filtrate was concentrated in vacuo to give the crude residue. The crude residue was purified by column chromatography (SiO₂, Petroleum ether:Ethyl acetate=1/0 to 5/1) to give the title compound (11 g, 40% yield) as a yellow solid. LC-MS (ESI⁺) m/z 230.1 (M+H)⁺; ¹H NMR (400 MHz, DMSO-d6) δ=7.25 (d, J=8.8 Hz, 1H), 6.35 (br d, J=2.4 Hz, 1H), 6.22 (br dd, J=2.4, 8.8 Hz, 1H), 3.81 (s, 3H), 2.90 (s, 6H).

### Step 2—3-Methoxy-N,N-dimethyl-4-(4,4,5,5-tetramethyl-1,3,2-dioxaborolan-2-yl)aniline

A mixture of 4-bromo-3-methoxy-N,N-dimethylaniline (1 g, 4 mmol), 4,4,4′,4′,5,5,5′,5′-octamethyl-2,2′-bi(1,3,2-dioxaborolane) (2.21 g, 8.69 mmol), KOAc (1.28 g, 13 mmol), and Pd(dppf)Cl₂·CH₂Cl₂ (355 mg, 435 μmol) in dioxane (20 mL) was degassed and purged with N₂ three times. Then the mixture was stirred at 80° C. for 2 hrs under N₂ atmosphere. On completion, the reaction mixture was concentrated in vacuo to give the crude residue. The crude residue was purified by column chromatography (SiO₂, Petroleum ether:Ethyl acetate=1/0 to 5/1) to give the title compound (440 mg, 33% yield) as a red oil. LC-MS (ESI⁺) m/z 278.1 (M+H)⁺.

### 7-Bromo-5-chloro-N,N-dimethylbenzofuran-2-carboxamide (Intermediate HX)

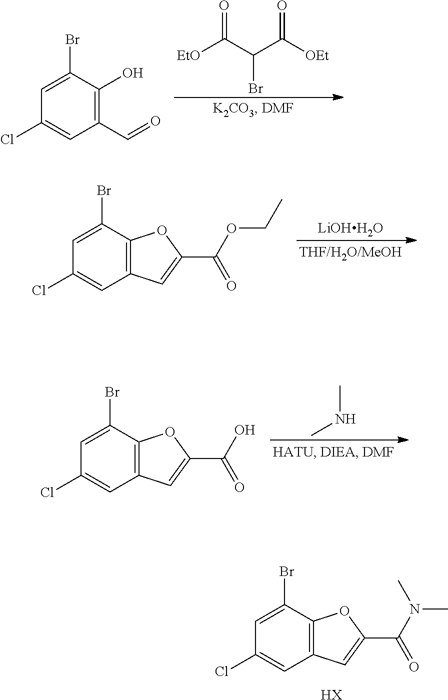

### Step 1—Ethyl 7-bromo-5-chlorobenzofuran-2-carboxylate

To a solution of 3-bromo-5-chloro-2-hydroxybenzaldehyde (5 g, 20 mmol, CAS #19652-32-5) and diethyl 2-bromomalonate (10.2 g, 42.4 mmol, CAS #685-87-0) in DMF (30 mL) was added K₂CO₃ (5.87 g, 42.5 mmol). The mixture was then stirred at 80° C. for 12 hrs. On completion, the reaction mixture was diluted with water (50 mL) and extracted with EA (50 mL×3). The combined organic layers were washed with brine (40 mL×2), dried over Na₂SO₄, filtered and concentrated under reduced pressure to give a residue. The residue was purified by column chromatography (SiO₂, Petroleum ether:Ethyl acetate=1/0 to 20/1) to give the title compound (3.5 g, 49% yield) as a white solid.

### Step 2—7-Bromo-5-chlorobenzofuran-2-carboxylic acid

To a solution of ethyl 7-bromo-5-chlorobenzofuran-2-carboxylate (2 g, 7 mmol) in THF (3 mL) and H₂O (1 mL) and MeOH (1 mL) was added LiOH·H₂O (2.76 g, 65.9 mmol). The mixture was then stirred at 20° C. for 2 hrs. On completion, the reaction mixture was added HCl (1 mol) until the pH was 7, then diluted by water (30 mL) and extracted by ethyl acetate (3×30 mL). The combined organic layers were dried over anhydrous sodium sulfate, filtered and the filtrate was concentrated in vacuo to give the title compound (2 g) as a white solid. LC-MS (ESI⁺) m/z 274.7 (M+H)⁺.

### Step 3—7-Bromo-5-chloro-N,N-dimethylbenzofuran-2-carboxamide

To a solution of 7-bromo-5-chlorobenzofuran-2-carboxylic acid (845 mg, 3.07 mmol) in DMF (10 mL) was added HATU (1.40 g, 3.68 mmol) and DIEA (1.98 g, 15.3 mmol), was added N-methylmethanamine;hydrochloride (724 mg, 6.13 mmol, CAS #124-40-3). Then the mixture was stirred at 0° C. for 4 hrs. On completion, the mixture was poured into cold water (25 mL) and triturated for 0.5 hr, then the mixture was filtered and the filter cake was dried in vacuo to give the title compound (1 g) as a yellow solid.

### 7-(2-Methoxyphenyl)-N,N-dimethyl-5-(1,2,5,6-tetrahydropyridin-3-yl)benzofuran-2-carboxamide (Intermediate HY)

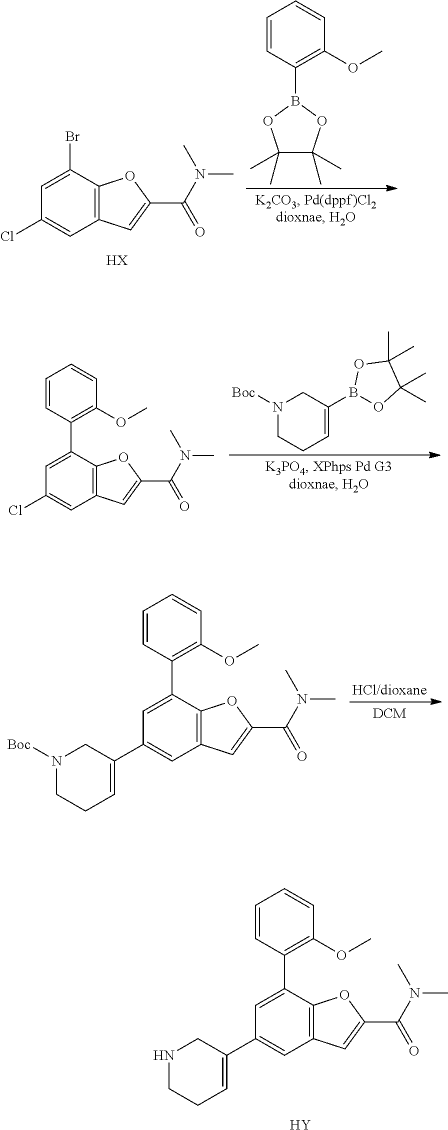

### Step 1—5-Chloro-7-(2-methoxyphenyl)-N,N-dimethylbenzofuran-2-carboxamide

A mixture of 7-bromo-5-chloro-N,N-dimethylbenzofuran-2-carboxamide (500 mg, 1.65 mmol, Intermediate HX), 2-(2-methoxyphenyl)-4,4,5,5-tetramethyl-1,3,2-dioxaborolane (301 mg, 1.98 mmol, CAS #190788-60-4), and K₂CO₃ (685 mg, 4.96 mmol) in dioxane (3 mL) and H₂O (1 mL) was degassed and purged with N₂ three times. Then Pd(dppf)Cl₂ (121 mg, 165 μmol) was added and the mixture was stirred at 60° C. for 2 hrs under N₂ atmosphere. On completion, the reaction mixture was concentrated under reduced pressure to give a residue. The residue was purified by column chromatography (SiO₂, Petroleum ether:Ethyl acetate=10/1 to 0/1) to give the title compound (0.4 g, 71% yield) as a yellow oil. LC-MS (ESI⁺) m/z 230.2 (M+H)⁺.

### Step 2—Tert-butyl 3-(2-(dimethylcarbamoyl)-7-(2-methoxyphenyl)benzofuran-5-yl)-5,6-dihydropyridine-1(2H)-carboxylate

A mixture of 5-chloro-7-(2-methoxyphenyl)-N,N-dimethylbenzofuran-2-carboxamide (0.2 g, 606 μmol), tert-butyl 3-(4,4,5,5-tetramethyl-1,3,2-dioxaborolan-2-yl)-5,6-dihydropyridine-1(2H)-carboxylate (281 mg, 909 μmol), XPhos Pd G3 (51.3 mg, 60.6 μmol), and K₃PO₄ (386 mg, 1.82 mmol) in dioxane (3 mL) and H₂O (1 mL) was degassed and purged with N₂ three times. Then the mixture was stirred at 80° C. for 1 hr under N₂ atmosphere. On completion, the reaction mixture was concentrated under reduced pressure to give a residue. The residue was purified by column chromatography (SiO₂, Dichloromethane: Methanol=20/1 to 10/1) to give the title compound as a white solid. (135 mg, 46% yield). LC-MS (ESI⁺) m/z 477.3 (M+H)⁺.

### Step 3—7-(2-Methoxyphenyl)-N,N-dimethyl-5-(1,2,5,6-tetrahydropyridin-3-yl)benzofuran-2-carboxamide

To a solution of tert-butyl 3-(2-(dimethylcarbamoyl)-7-(2-methoxyphenyl)benzofuran-5-yl)-5,6-dihydropyridine-1(2H)-carboxylate in DCM (5 mL) was added HCl/dioxane (4.0 M, 5.0 mL), then the mixture was stirred at 25° C. for 1 h. On completion, the mixture was concentrated to give the title compound (130 mg, HCl) as a yellow solid. LC-MS (ESI⁺) m/z 377.3 (M+H)⁺.

### 6-(1-(3-(1H-1,2,3-triazol-1-yl)propanoyl)-1,2,5,6-tetrahydropyridin-3-yl)-4-(4-ethylpyridin-3-yl)-7-fluoro-1H-indole-2-carboxylic acid (Intermediate HZ)

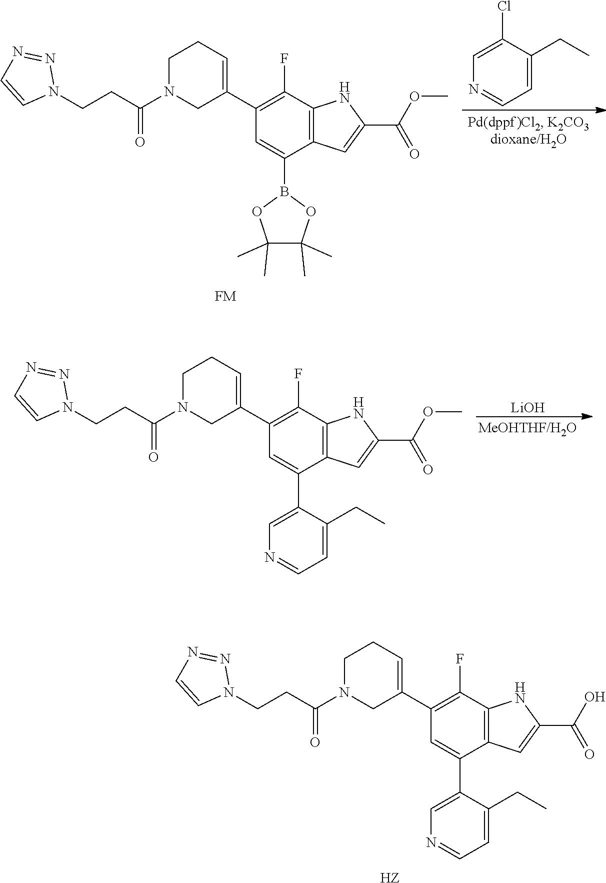

### Step 1—Methyl 6-(1-(3-(1H-1,2,3-triazol-1-yl)propanoyl)-1,2,5,6-tetrahydropyridin-3-yl)-4-(4-ethylpyridin-3-yl)-7-fluoro-1H-indole-2-carboxylate

To a solution of methyl 7-fluoro-4-(4,4,5,5-tetramethyl-1,3,2-dioxaborolan-2-yl)-6-[1-[3-(triazol-1-yl)propanoyl]-3,6-dihydro-2H-pyridin-5-yl]-1H-indole-2-carboxylate (2 g, 3.82 mmol, Intermediate FM) and 3-bromo-4-ethyl-pyridine (710 mg, 3.82 mmol) in dioxane (20 mL) and H₂O (4 mL) was added Pd(dppf)Cl₂ (279 mg, 382 μmol) and K₂CO₃ (1.58 g, 11.46 mmol). The mixture was then stirred at 80° C. for 1 hr under N₂ atmosphere. The reaction mixture was filtered and concentrated under reduced pressure to give a residue. The crude product was triturated with ethyl acetate at 25° C. for 10 mins to give the title compound (1.3 g, 55% yield) as a brown solid. LC-MS (ESI+) m/z 503.2 (M+H)⁺.

### Step 2—6-(1-(3-(1H-1,2,3-triazol-1-yl)propanoyl)-1,2,5,6-tetrahydropyridin-3-yl)-4-(4-ethylpyridin-3-yl)-7-fluoro-1H-indole-2-carboxylic acid

To a solution of methyl 4-(4-ethyl-3-pyridyl)-7-fluoro-6-[1-[3-(triazol-1-yl)propanoyl]-3,6-dihydro-2H-pyridin-5-yl]-1H-indole-2-carboxylate (1.3 g, 2.6 mmol) in THF (4 mL), H₂O (4 mL) and MeOH (4 mL) was added LiOH·H₂O (542 mg, 12.93 mmol). The mixture was then stirred at 25° C. for 12 hrs. On completion, the mixture was filtered and concentrated under reduced pressure to give a residue. The residue was extracted with EA (30 mL×3). The aqueous phase was adjusted to pH of 5 and extracted with DCM (100 mL×3). The combined organic layers were dried over Na₂SO₄, filtered and concentrated under reduced pressure to give a residue. The residue was purified by prep-HPLC (HCl condition) to give the title compound (1.3 g, 86% yield, HCl) as a off-brown solid. LC-MS (ESI+) m/z 489.2 (M+H)⁺.

### 3-Methoxy-4-(piperazin-1-yl)benzonitrile (Intermediate IA)

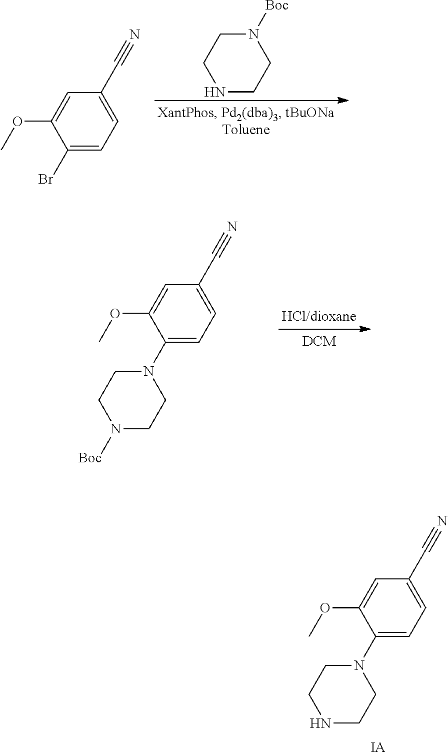

### Step 1—Tert-butyl 4-(4-cyano-2-methoxyphenyl)piperazine-1-carboxylate

A mixture of 4-bromo-3-methoxybenzonitrile (5 g, 24 mmol, CAS #120315-65-3), tert-butyl piperazine-1-carboxylate (5.27 g, 28.3 mmol), t-BuONa (4.53 g, 47.2 mmol), XPhos (1.12 g, 2.36 mmol) and Pd₂(dba)₃ (1.08 g, 1.18 mmol) in dioxane (100 mL) was degassed and purged with N₂ three times. Then the mixture was stirred at 100° C. for 4 hrs under N₂ atmosphere. On completion, the residue was purified by column chromatography (SiO₂, Petroleum ether:Ethyl acetate=1/0 to 1/1) to give the title compound (2.5 g, 25% yield) as a yellow solid. LC-MS (ESI⁺) m/z 318.0 (M+H)⁺.

### Step 2—3-Methoxy-4-(piperazin-1-yl)benzonitrile

To a solution of tert-butyl 4-(4-cyano-2-methoxyphenyl)piperazine-1-carboxylate (1 g, 3.15 mmol) in DCM (5 mL) was added HCl/dioxane (4 M, 5 mL). The mixture was then stirred at 25° C. for 0.5 hrs. On completion, the reaction mixture was concentrated in vacuo to give the title compound (500 mg, HCl) as a yellow solid. LC-MS (ESI⁺) m/z 218.1 (M+H)⁺.

### 1-(3-Fluoro-2-methoxyphenyl)piperazine (CAS #1121613-46-4) (Intermediate IB)

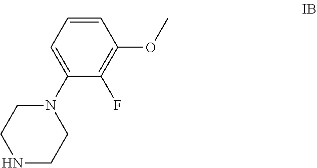

### 1-(5-(4-chloro-7-fluoro-2-(4-(5-fluoro-3-methoxypyridin-2-yl)piperazine-1-carbonyl)-1H-indol-6-yl)-3,6-dihydropyridin-1(2H)-yl)-3-(1H-1,2,3-triazol-1-yl)propan-1-one (Intermediate IC)

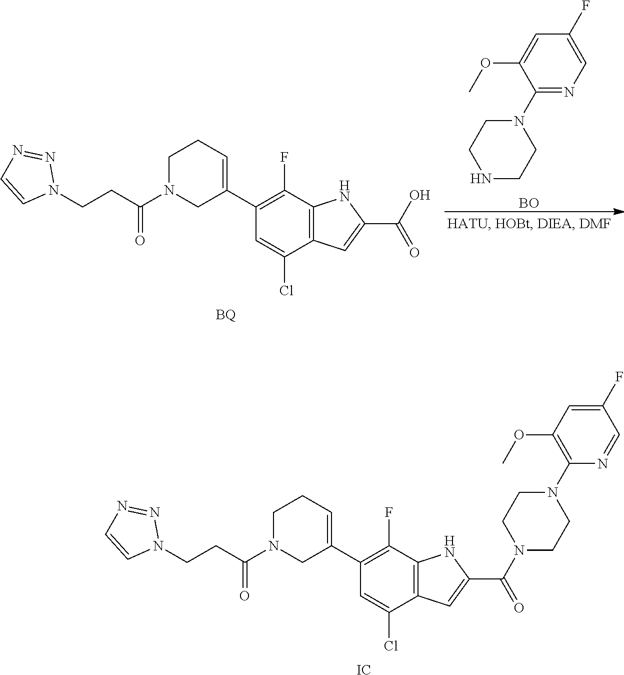

To a solution of 4-chloro-7-fluoro-6-[1-[3-(triazol-1-yl)propanoyl]-3,6-dihydro-2H-pyridin-5-yl]-1H-indole-2-carboxylic acid (5.6 g, 13.40 mmol, Intermediate BQ) in DMF (60 mL) was added HOBt (3.62 g, 26.81 mmol), DIEA (5.20 g, 40.21 mmol, 7.00 mL), and HATU (6.12 g, 16.08 mmol) at 0° C. for 15 min. Then 1-(5-fluoro-3-methoxy-2-pyridyl) piperazine (3.32 g, 13.40 mmol, HCl, Intermediate BO) was added to the mixture and the mixture was stirred at 20° C. for 0.5 hr. On completion, the reaction was quenched with H₂O (120 mL). During this period, yellow precipitate was formed, which was washed with water (50 mL) and collected by filtration. The filter cake was dried in vacuum. The crude product was triturated with Petroleum ether/Ethyl acetate=1/1 (20 mL) at 20° C. for 30 min, then filtered and dried to give the title compound (7.3 g, 89% yield) as yellow solid. ¹H NMR (400 MHz, DMSO-d₆) δ=12.53 (br d, J=3.2 Hz, 1H) 8.11 (br d, J=8.0 Hz, 1H) 7.80 (d, J=2.0 Hz, 1H) 7.69 (br d, J=8.4 Hz, 1H) 7.46-7.59 (m, 1H) 7.37 (m, 1H) 7.02-7.19 (m, 1H) 6.80 (br s, 1H) 6.14 (br d, J=9.6 Hz, 1H) 4.62 (m, 2H) 4.30 (br d, J=19.2 Hz, 2H) 3.80-3.87 (m, 5H) 3.54-3.66 (m, 2H) 3.29 (br s, 4H) 3.05-3.14 (m, 2H) 2.20-2.39 (m, 2H) 1.25 (br d, J=6.6 Hz, 1H).

### 6-(1-(3-(1H-1,2,3-triazol-1-yl)propanoyl)-1,2,5,6-tetrahydropyridin-3-yl)-4-(2-cyclopropoxyphenyl)-7-fluoro-1H-indole-2-carboxylic acid (Intermediate ID)

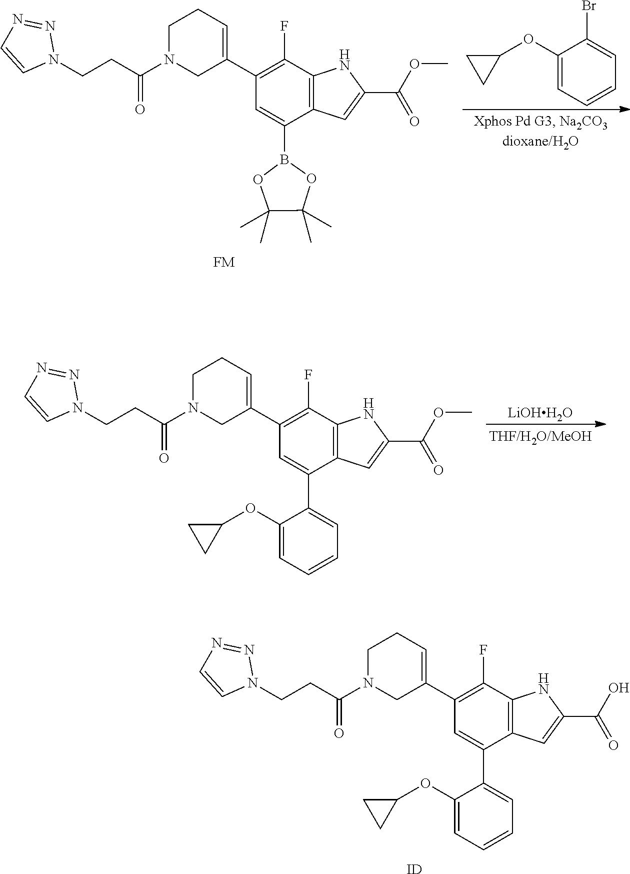

### Step 1—Methyl 6-(1-(3-(1H-1,2,3-triazol-1-yl)propanoyl)-1,2,5,6-tetrahydropyridin-3-yl)-4-(2-cyclopropoxyphenyl)-7-fluoro-1H-indole-2-carboxylate

A mixture of methyl 7-fluoro-4-(4,4,5,5-tetramethyl-1,3,2-dioxaborolan-2-yl)-6-[1-[3-(triazol-1-yl)propanoyl]-3,6-dihydro-2H-pyridin-5-yl]-1H-indole-2-carboxylate (220 mg, 420 μmol, Intermediate FM), 1-bromo-2-(cyclopropoxy)benzene (134 mg, 631 μmol. CAS #38380-86-8), 1-bromo-2-(cyclopropoxy)benzene (134 mg, 630 μmol), XPhos Pd G3 (35.6 mg, 42 μmol), and Na₂CO₃ (134 mg, 1.26 mmol) in dioxane (5 mL) and H₂O (1 mL) was degassed and purged with N₂ three times. Then the mixture was stirred at 60° C. for 1 hr under N₂ atmosphere. On completion, the mixture was quenched by addition of H₂O (5 mL), and then diluted with EA (10 mL) and extracted with EA (30 mL×2). The combined organic layers were washed with NaCl (20 mL×2), dried over Na₂SO₄, filtered and concentrated under reduced pressure to give a residue. The residue was purified by column chromatography (SiO₂, Petroleum ether/Ethyl acetate=10/1 to 0/1) to give the title compound (120 mg, 42% yield) as a white solid. LC-MS (ESI+) m/z 530.2 (M+H)⁺.

### Step 2—6-(1-(3-(1H-1,2,3-triazol-1-yl)propanoyl)-1,2,5,6-tetrahydropyridin-3-yl)-4-(2-cyclopropoxyphenyl)-7-fluoro-1H-indole-2-carboxylic acid

To a solution of methyl 4-[2-(cyclopropoxy)phenyl]-7-fluoro-6-[1-[3-(triazol-1-yl)propanoyl]-3,6-dihydro-2H-pyridin-5-yl]-1H-indole-2-carboxylate (90 mg, 170 μmol) in THF (0.3 mL), H₂O (0.3 mL) and MeOH (0.3 mL) was added LiOH·H₂O (21.4 mg, 510 μmol). The mixture then was stirred at 40° C. for 1 hr. On completion, the crude product was purified by reversed-phase HPLC(0.1% FA condition) to give the title compound (60 mg, 68% yield) as a white solid. LC-MS (ESI+) m/z 516.2 (M+H)⁺.

### 2-Bromo-5-fluoro-3-methoxypyridine (CAS #1256806-73-1) (Intermediate IE)

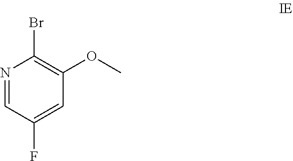

### 4-Bromo-3-ethylpyridine (CAS #10168-60-2) (Intermediate IF)

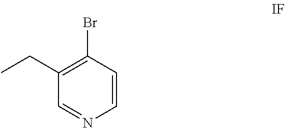

### 1-(3-(7-Fluoro-2-(4-(5-fluoro-3-methoxypyridin-2-yl)piperazine-1-carbonyl)-4-(4,4,5,5-tetramethyl-1,3,2-dioxaborolan-2-yl)-1H-indol-6-yl)-5,6-dihydropyridin-1(2H)-yl)-3-(1H-1,2,3-triazol-1-yl)propan-1-one (Intermediate IG)

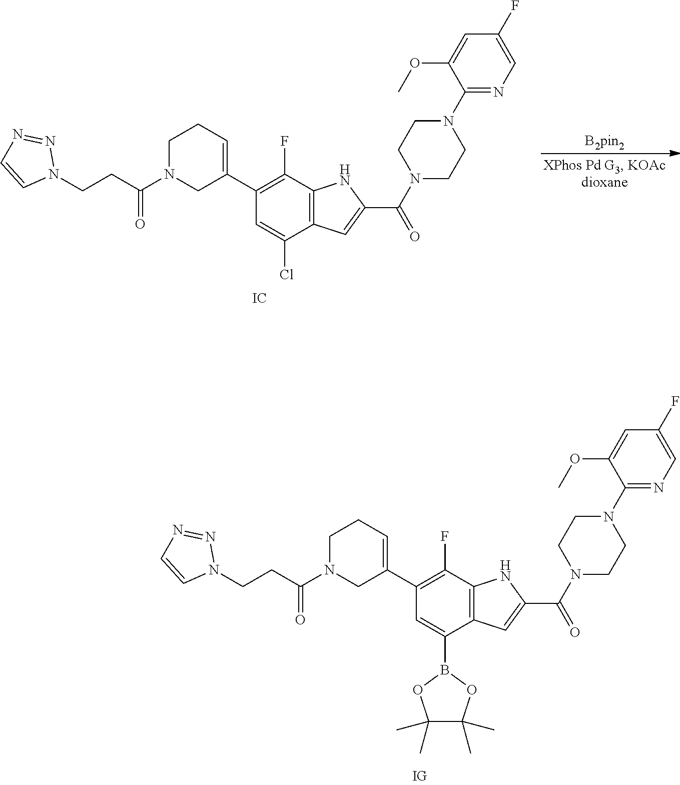 [SMILES: COc1cc(F)cnc1N1CCN(C(=O)c2cc3c(Cl)cc(C4=CCCN(C(=O)CCn5ccnn5)C4)c(F)c3[nH]2)CC1 ; "IC" ; COc1cc(F)cnc1N1CCN(C(=O)c2cc3c(B4OC(C)(C)C(C)(C)O4)cc(C4=CCCN(C(=O)CCn5ccnn5)C4)c(F)c3[nH]2)CC1]

A mixture of 1-[5-[4-chloro-7-fluoro-2-[4-(5-fluoro-3-methoxy-2-pyridyl)piperazine-1-carbonyl]-1H-indol-6-yl]-3,6-dihydro-2H-pyridin-1-yl]-3-(triazol-1-yl)propan-1-one (500 mg, 818 μmol, Intermediate IC), 4,4,5,5-tetramethyl-2-(4,4,5,5-tetramethyl-1,3,2-dioxaborolan-2-yl)-1,3,2-dioxaborolane (270 mg, 1.06 mmol), XPhos Pd G3 (69.2 mg, 81.8 μmol), and KOAc (240 mg, 2.45 mmol) in dioxane (5 mL) was degassed and purged with N₂ three times. Then the mixture was stirred at 80° C. for 2 h under N₂ atmosphere. On completion, the crude product was triturated with H₂O at 0° C. for 15 min to give the title compound (0.5 g) as a white solid. LC-MS (ESI⁺) m/z=703.2 (M+H)⁺.

### Tert-butyl 4-(4-cyclopropyl-2-methoxyphenyl)piperazine-1-carboxylate (Intermediate IH)

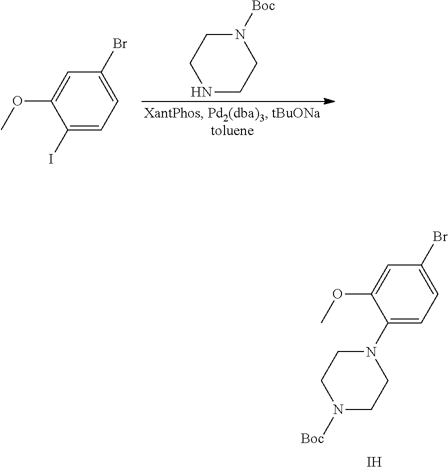

A mixture of 4-bromo-1-iodo-2-methoxybenzene (2 g, 6 mmol, CAS #791642-68-7), tert-butyl piperazine-1-carboxylate (1.19 g, 6.39 mmol, CAS #57260-71-6), Xantphos (369 mg, 639 μmol), Pd₂(dba)₃ (292 mg, 319 μmol) and tBuONa (1.84 g, 19.1 mmol) in toluene (10 mL) was degassed and purged with N₂ three times. Then the mixture was stirred at 60° C. for 3 hrs under N₂ atmosphere. On completion, the reaction mixture was concentrated under reduced pressure and purified by column chromatography (SiO₂, Petroleum ether/Ethyl acetate=1/0 to 1/1) to give the title compound (2 g, 84% yield) as a white solid. LC-MS (ESI⁺) m/z 371.1 (M+H)⁺.

### 1-(4-Cyclopropyl-2-methoxyphenyl)piperazine (Intermediate II)

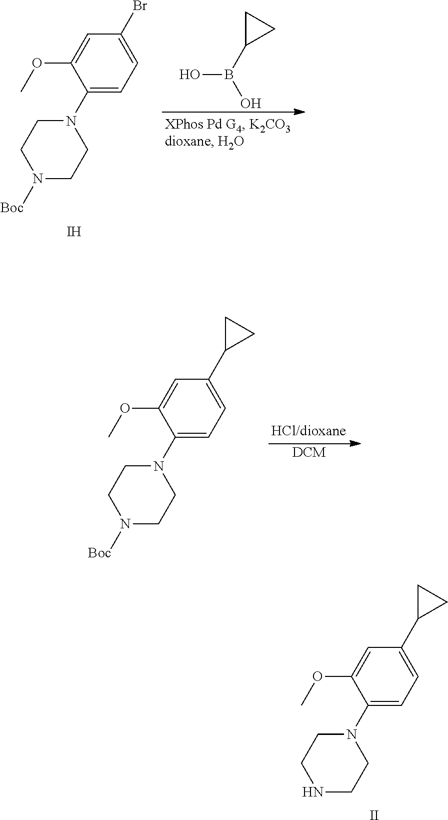

### Step 1—Tert-butyl 4-(4-cyclopropyl-2-methoxyphenyl)piperazine-1-carboxylate

A mixture of tert-butyl 4-(4-bromo-2-methoxyphenyl)piperazine-1-carboxylate (0.5 g, 1.4 mmol, Intermediate IH), cyclopropylboronic acid (347 mg, 4.04 mmol, CAS #411235-57-9), K₂CO₃ (558 mg, 4.04 mmol), and Xphos Pd G4 (115 mg, 134 μmol) in H₂O (0.5 mL) and dioxane (2 mL) was degassed and purged with N₂ three times. Then the mixture was stirred at 90° C. for 12 hrs under N₂ atmosphere. On completion, the reaction mixture was filtered and concentrated under reduced pressure and purified by column chromatography (SiO₂, Petroleum ether/Ethyl acetate=1/1 to 0/1) to give the title compound (0.3 g, 60% yield) as a white solid. LC-MS (ESI⁺) m/z 355.0 (M+23)⁺.

### Step 2—1-(4-Cyclopropyl-2-methoxyphenyl)piperazine

To a solution of tert-butyl 4-(4-cyclopropyl-2-methoxyphenyl)piperazine-1-carboxylate (0.30 g, 902 μmol) in DCM (4 mL) was added TFA (102 mg, 902 μmol). The mixture was stirred at 25° C. for 2 hrs. On completion, the reaction mixture was filtered and concentrated under reduced pressure to give the title compound (250 mg, TFA) as a red solid. LC-MS (ESI⁺) m/z 233.0 (M+H)⁺.

### 3-Bromo-4-ethylpyridine (CAS #38749-76-7) (Intermediate IJ)

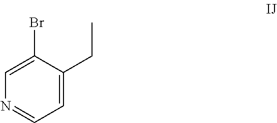

### (6-Bromo-4-chloro-7-fluoro-1H-indol-2-yl)(4-(3-methoxypyridin-2-yl)piperazin-1-yl)methanone (Intermediate IK)

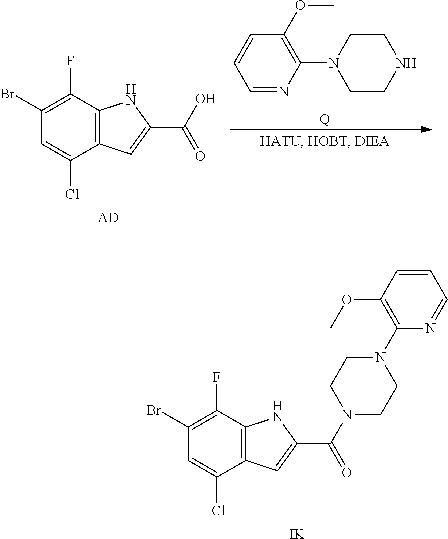

To a solution of 6-bromo-4-chloro-7-fluoro-1H-indole-2-carboxylic acid (1 g, 3 mmol, Intermediate AD) in DMF (10 mL) was added HATU (1.56 g, 4.10 mmol), HOBt (693 mg, 5.13 mmol), DIEA (2.21 g, 17.1 mmol, 2.98 mL) and 1-(3-methoxypyridin-2-yl)piperazine (661 mg, 3.42 mmol, Intermediate Q). The mixture was then stirred at 25° C. for 0.5 hr. On completion, the reaction mixture was poured into water (100 ml) filtered and concentrated under reduced pressure to give the title compound (1.3 g) as a yellow solid. LC-MS (ESI⁺) m/z 466.9 (M+H)⁺.

### 1-(5-(4-Bromo-7-fluoro-2-(4-(3-methoxypyridin-2-yl)piperazine-1-carbonyl)-1H-indol-6-yl)-3,6-dihydropyridin-1(2H)-yl)ethan-1-one (Intermediate IL)

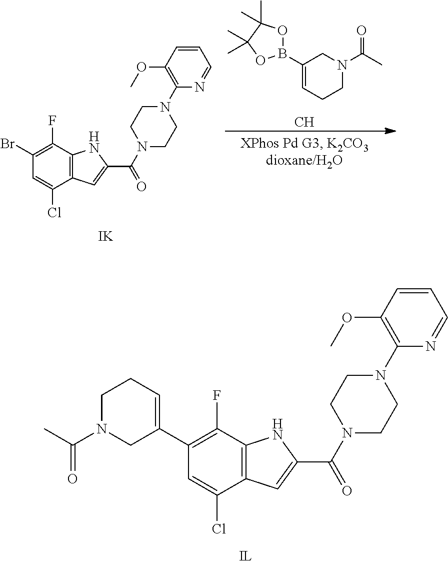

To a solution of (6-bromo-4-chloro-7-fluoro-1H-indol-2-yl)(4-(3-methoxypyridin-2-yl)piperazin-1-yl)methanone (1.2 g, 2.57 mmol, Intermediate IK) and 1-(5-(4,4,5,5-tetramethyl-1,3,2-dioxaborolan-2-yl)-3,6-dihydropyridin-1(2H)-yl)ethan-1-one (644 mg, 2.57 mmol, Intermediate CH) in dioxane (10 mL) and H₂O (2 mL) was added XPhos Pd G3 (217 mg, 257 μmol) and K₂CO₃ (1.06 g, 7.70 mmol). The mixture was then stirred at 80° C. for 2 hrs. On completion, the reaction mixture was diluted with water (200 mL) and extracted with ethyl dichloromethane (3×50 mL). The combined organic layers were washed with brine (200 mL) and dried over anhydrous sodium sulfate, filtered and the filtrate was concentrated in vacuo to give the crude residue. The residue was purified by column chromatography (SiO₂, Petroleum ether:Ethyl acetate=5/1 to 0/1) to give the title compound (750 mg, 46% yield) as a yellow solid. LC-MS (ESI⁺) m/z 512.2 (M+H)⁺.

### 1-(5-(7-Fluoro-2-(4-(3-methoxypyridin-2-yl)piperazine-1-carbonyl)-4-(4,4,5,5-tetramethyl-1,3,2-dioxaborolan-2-yl)-1H-indol-6-yl)-3,6-dihydropyridin-1(2H)-yl)ethan-1-one (Intermediate IM)

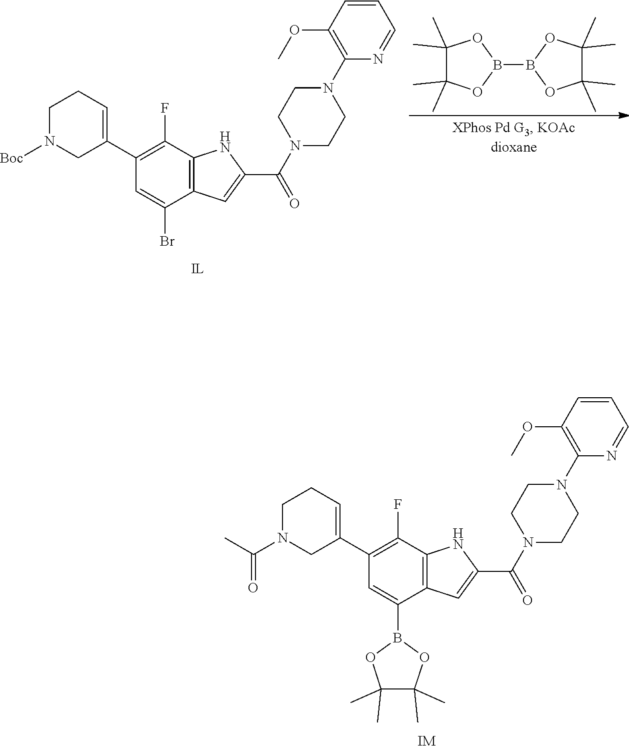

A mixture of 1-(5-(4-bromo-7-fluoro-2-(4-(3-methoxypyridin-2-yl)piperazine-1-carbonyl)-1H-indol-6-yl)-3,6-dihydropyridin-1(2H)-yl)ethan-1-one (690 mg, 1.35 mmol, Intermediate IL) and 4,4,4′,4′,5,5,5′,5′-octamethyl-2,2′-bi(1,3,2-dioxaborolane) (1.03 g, 4.04 mmol) in dioxane (15 mL) was added XPhos Pd G3 (114.09 mg, 134.79 μmol) and KOAc (397 mg, 4.04 mmol). The mixture was then stirred at 80° C. for 2 hrs. On completion, the reaction mixture was filtered and the filtrate was concentrated in vacuo to give the crude residue. The residue was purified by column chromatography (SiO₂, Petroleum ether:Ethyl acetate=5/1 to THF=1) to give the title compound (580 mg, 62% yield) as a yellow solid. LC-MS (ESI⁺) m/z 604.4 (M+H)⁺.

### 1-Methyl-5-phenyl-4-(4,4,5,5-tetramethyl-1,3,2-dioxaborolan-2-yl)-1H-pyrazole (CAS #2684308-79-8) (Intermediate IN)

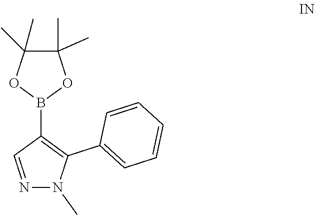 [SMILES: Cn1ncc(B2OC(C)(C)C(C)(C)O2)c1-c1ccccc1]

### 3-(4,4,5,5-Tetramethyl-1,3,2-dioxaborolan-2-yl)phenol (CAS #214360-76-6) (Intermediate IO)

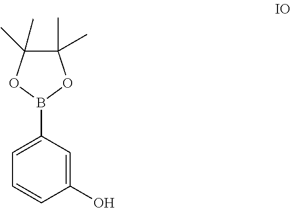 [SMILES: CC1(C)OB(c2cccc(O)c2)OC1(C)C]

### (2-Methyl-3-(4,4,5,5-tetramethyl-1,3,2-dioxaborolan-2-yl)phenyl)methanol (CAS #1400755-05-6) (Intermediate IP)

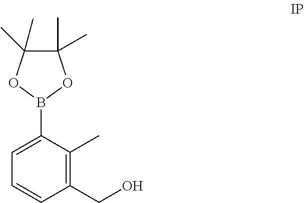 [SMILES: Cc1c(CO)cccc1B1OC(C)(C)C(C)(C)O1]

### 1-Methyl-4-(4-(4,4,5,5-tetramethyl-1,3,2-dioxaborolan-2-yl)pyridin-2-yl)piperazine (CAS #918524-63-7) (Intermediate IQ)

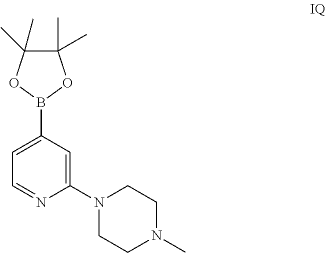

### (2,4-diethoxyphenyl)boronic acid (CAS #1072952-01-2) (Intermediate IR)

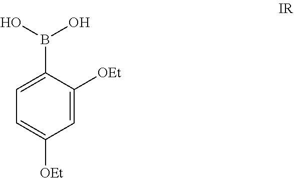 [SMILES: CCOc1ccc(B(O)O)c(OCC)c1]

### 3,5-Dimethyl-1-(3-(4,4,5,5-tetramethyl-1,3,2-dioxaborolan-2-yl)phenyl)-1H-pyrazole (CAS #2674178-86-8) (Intermediate IS)

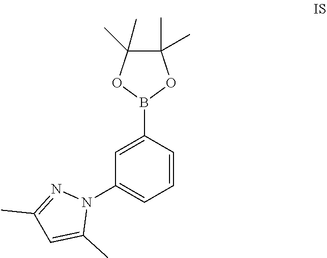

### 3-Cyclopropyl-1-methyl-5-(4,4,5,5-tetramethyl-1,3,2-dioxaborolan-2-yl)-1H-indazole (CAS #1904650-82-3) (Intermediate IT)

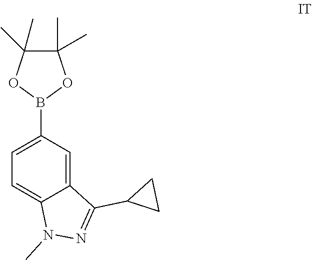 [SMILES: Cn1nc(C2CC2)c2cc(B3OC(C)(C)C(C)(C)O3)ccc21]

### 1,3-Dimethyl-6-(4,4,5,5-tetramethyl-1,3,2-dioxaborolan-2-yl)-1H-indazole (CAS #1300582-61-9) (Intermediate IU)

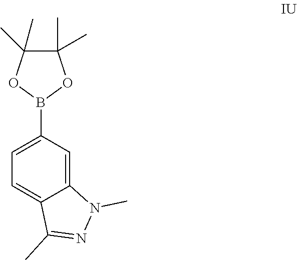

### 2-Methyl-5-(4,4,5,5-tetramethyl-1,3,2-dioxaborolan-2-yl)-2H-indazole (CAS #1189746-27-7) (Intermediate IV)

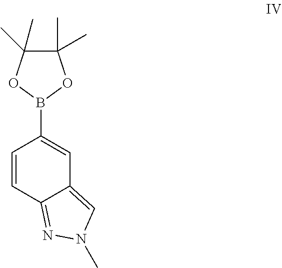

### 5-Methyl-1-phenyl-4-(4,4,5,5-tetramethyl-1,3,2-dioxaborolan-2-yl)-1H-pyrazole (CAS #849776-88-1) (Intermediate IW)

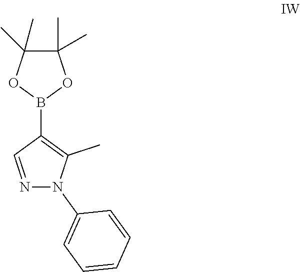 [SMILES: Cc1c(B2OC(C)(C)C(C)(C)O2)cnn1-c1ccccc1]

### 4-(3-(4,4,5,5-Tetramethyl-1,3,2-dioxaborolan-2-yl)phenyl)pyridine (CAS #1009033-83-3) (Intermediate IX)

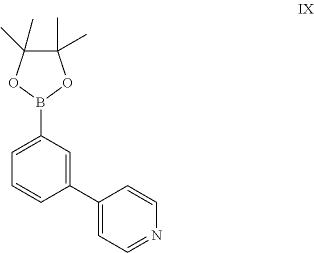 [SMILES: CC1(C)OB(c2cccc(-c3ccncc3)c2)OC1(C)C]

### 8-(4,4,5,5-tetramethyl-1,3,2-dioxaborolan-2-yl)isoquinoline (CAS #2379560-77-5) (Intermediate IY)

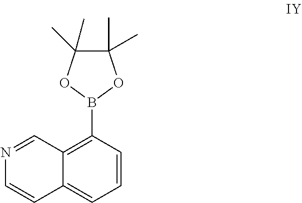 [SMILES: CC1(C)OB(c2cccc3ccncc23)OC1(C)C]

### 2-cyclopropyl-3-(4,4,5,5-tetramethyl-1,3,2-dioxaborolan-2-yl)pyridine (CAS #2098215-65-5) (Intermediate IZ)

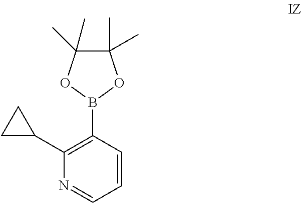 [SMILES: CC1(C)OB(c2cccnc2C2CC2)OC1(C)C]

### 2,6-dimethyl-4-(4,4,5,5-tetramethyl-1,3,2-dioxaborolan-2-yl)pyridine (CAS #325142-95-8) (Intermediate JA)

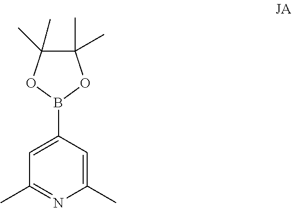

### 3-(3-(4,4,5,5-Tetramethyl-1,3,2-dioxaborolan-2-yl)phenyl)pyridine (CAS #939430-30-5) (Intermediate JB)

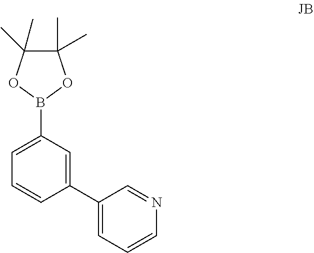 [SMILES: CC1(C)OB(c2cccc(-c3cccnc3)c2)OC1(C)C]

### 2-Methyl-4-(4,4,5,5-tetramethyl-1,3,2-dioxaborolan-2-yl)quinoline (CAS #1627721-83-8) (Intermediate JC)

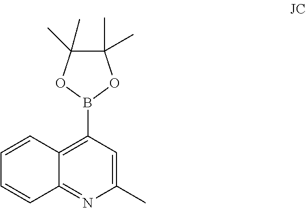 [SMILES: Cc1cc(B2OC(C)(C)C(C)(C)O2)c2ccccc2n1]

### 7(7-fluoro-2-methylquinolin-8-yl)boronic acid (CAS #1072945-61-9) (Intermediate JD)

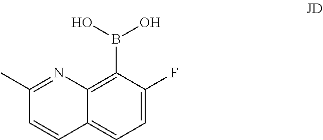

### 6-Methyl-8-(4,4,5,5-tetramethyl-1,3,2-dioxaborolan-2-yl)quinoline (CAS #2096334-29-9) (Intermediate JE)

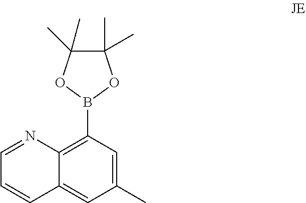 [SMILES: Cc1cc(B2OC(C)(C)C(C)(C)O2)c2ncccc2c1]

### (2-(prop-1-yn-1-yl)pyridin-4-yl)boronic acid (CAS #1189373-05-4) (Intermediate JF)

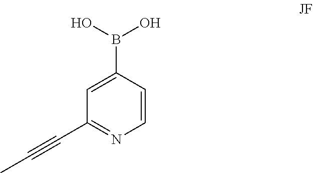

### 8-Methyl-5-(4,4,5,5-tetramethyl-1,3,2-dioxaborolan-2-yl)quinoline (CAS #1366740-47-7) (Intermediate JG)

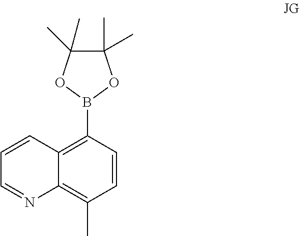 [SMILES: Cc1ccc(B2OC(C)(C)C(C)(C)O2)c2cccnc12]

### 2-Methyl-5-(4,4,5,5-tetramethyl-1,3,2-dioxaborolan-2-yl)quinoline (CAS #876922-75-7) (Intermediate JG)

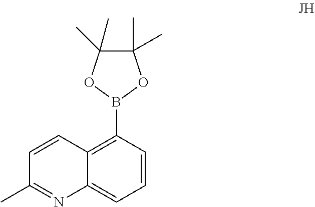

### 3-Cyclopropyl-5-(4,4,5,5-tetramethyl-1,32-dioxaborolan-2-yl)pyridine (CAS #1220696-43-4) (Intermediate JI)

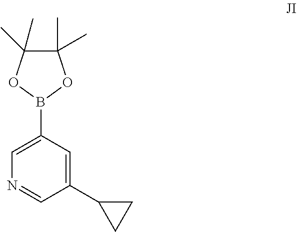 [SMILES: CC1(C)OB(c2cncc(C3CC3)c2)OC1(C)C]

### 2-(3-Isopropoxyphenyl)-4,4,5,5-tetramethyl-1,3,2-dioxaborolane (CAS #925916-59-2) (Intermediate JJ)

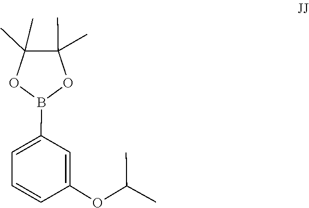 [SMILES: CC(C)Oc1cccc(B2OC(C)(C)C(C)(C)O2)c1]

### 2-(2,5-dihydrofuran-3-yl)-4,4,5,5-tetramethyl-1,3,2-dioxaborolane (CAS #212127-80-5) (Intermediate JK)

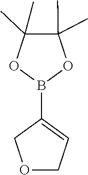

### ((3R,5S)-3,5-dimethylcyclohex-1-en-1-yl)boronic acid (CAS #1394909-88-6) (Intermediate JL)

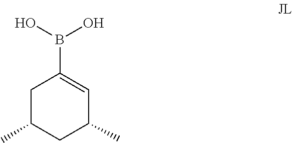

### (2-Cyclopropyl-4-methoxyphenyl)boronic acid (CAS #2225173-99-7) (Intermediate JM)

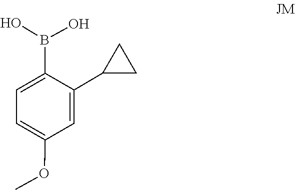

### (3-Ethoxy-4,5-difluorophenyl)boronic acid (CAS #1162261-96-2) (Intermediate JN)

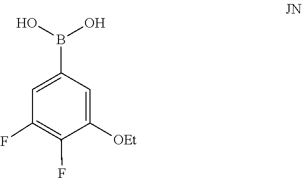 [SMILES: CCOc1cc(B(O)O)cc(F)c1F]

### 2-(2-Ethoxy-3-fluorophenyl)-4,4,5,5-tetramethyl-1,3,2-dioxaborolane (CAS #2246767-21-3) (Intermediate JO)

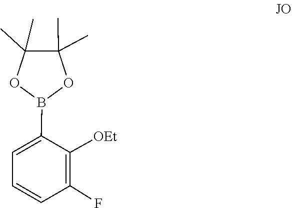 [SMILES: CCOc1c(F)cccc1B1OC(C)(C)C(C)(C)O1]

### 4,4,5,5-tetramethyl-2-((tetrahydro-4H-pyran-4-ylidene)methyl)-1,3,2-dioxaborolane (CAS #2304634-36-2) (Intermediate JP)

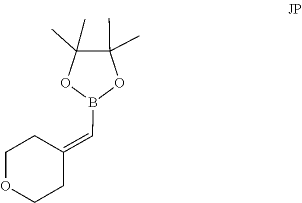 [SMILES: CC1(C)OB(C=C2CCOCC2)OC1(C)C]

### 2-(2-cyclopropylphenyl)-4,4,5,5-tetramethyl-1,3,2-dioxaborolane (CAS #1362243-53-5) (Intermediate JO)

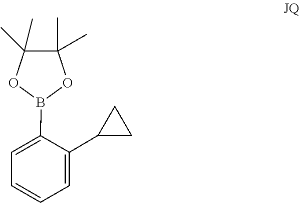 [SMILES: CC1(C)OB(c2ccccc2C2CC2)OC1(C)C]

### 2,5-Dimethyl-4-(4,4,5,5-tetramethyl-1,3,2-dioxaborolan-2-yl)pyridine (CAS #1309443-99-9) (Intermediate JR)

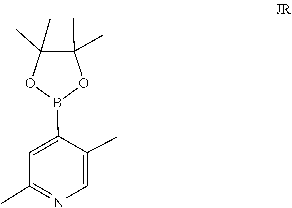

### 3-Phenyl-5-(4,4,5,5-tetramethyl-1,3,2-dioxaborolan-2-yl)pyridine (CAS #1171891-07-8) (Intermediate JS)

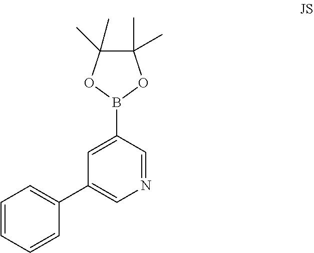 [SMILES: CC1(C)OB(c2cncc(-c3ccccc3)c2)OC1(C)C]

### 2-Methyl-5-(4,4,5,5-tetramethyl-1,3,2-dioxaborolan-2-yl)benzonitrile (CAS #1220219-11-3) (Intermediate JT)

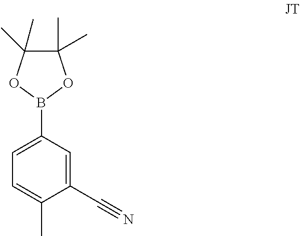 [SMILES: Cc1ccc(B2OC(C)(C)C(C)(C)O2)cc1C#N]

### (5-(1-Cyanocyclopropyl)pyridin-3-yl)boronic acid (CAS #1346264-25-2) (Intermediate JU)

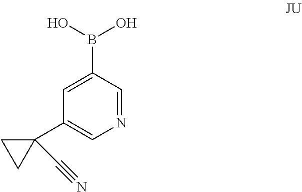 [SMILES: N#CC1(c2cncc(B(O)O)c2)CC1]

### (5-(1-cyanocyclopropyl)-2-fluorophenyl)boronic acid (CAS #1256345-50-2) (Intermediate JV)

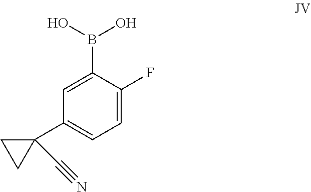 [SMILES: N#CC1(c2ccc(F)c(B(O)O)c2)CC1]

### (5-(Difluoromethoxy)pyridin-3-yl)boronic acid (CAS #1257554-65-6) (Intermediate JW)

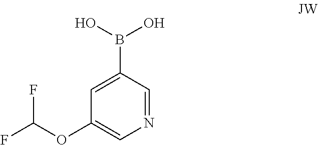 [SMILES: OB(O)c1cncc(OC(F)F)c1]

### 2-Propoxy-3-(4,4,5,5-tetramethyl-1,3,2-dioxaborolan-2-yl)pyridine (CAS #1073371-87-5) (Intermediate JX)

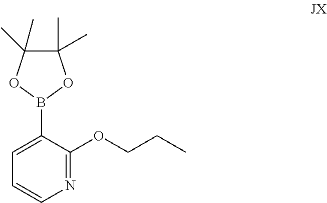 [SMILES: CCCOc1ncccc1B1OC(C)(C)C(C)(C)O1]

### 2-(Difluoromethoxy)-4-(4,4,5,5-tetramethyl-1,3,2-dioxaborolan-2-yl)pyridine (CAS #1628116-87-9) (Intermediate JY)

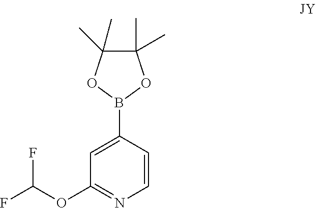 [SMILES: CC1(C)OB(c2ccnc(OC(F)F)c2)OC1(C)C]

### (5-methyl-2-propoxypyridin-3-yl)boronic acid (CAS #2096332-40-8) (Intermediate JZ)

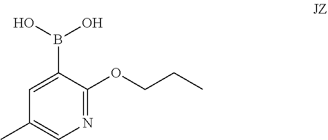

### 3-Isopropoxy-5-(4,4,5,5-tetramethyl-1,3,2-dioxaborolan-2-yl)pyridine (CAS #1171892-42-4) (Intermediate KA)

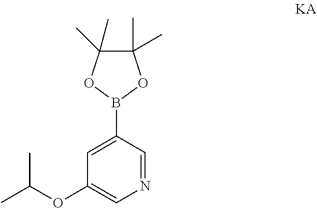 [SMILES: CC(C)Oc1cncc(B2OC(C)(C)C(C)(C)O2)c1]

### 2-Fluoro-3-methoxy-4-(4,4,5,5-tetramethyl-1,3,2-dioxaborolan-2-yl)pyridine CAS #2121513-86-6) (Intermediate KB)

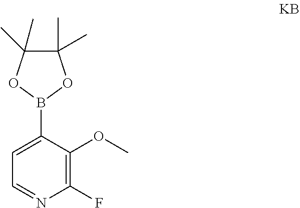

### 1-(4-(4,4,5,5-Tetramethyl-1,3,2-dioxaborolan-2-yl)-3,6-dihydropyridin-1(2H)-yl)ethan-1-one (CAS #1227068-67-8) (Intermediate KC)

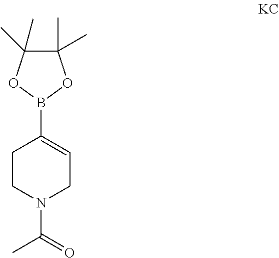

### (R)-4-(4,4,5,5-tetramethyl-1,3,2-dioxaborolan-2-yl)-2,3-dihydro-1H-inden-1-ol (CAS #2248640-93-7) (Intermediate KD)

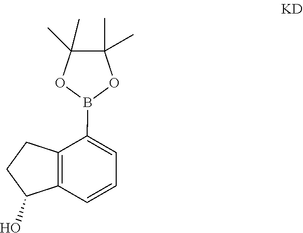 [SMILES: CC1(C)OB(c2cccc3c2CC[C@H]3O)OC1(C)C]

### 4-(4,4,5,5-Tetramethyl-1,3,2-dioxaborolan-2-yl)naphthalen-2-ol (CAS #2043962-01-0) (Intermediate KE)

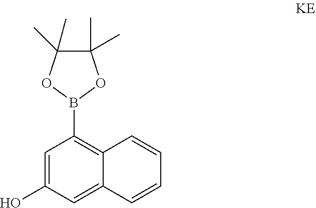 [SMILES: CC1(C)OB(c2cc(O)cc3ccccc23)OC1(C)C]

### 2-(5-Fluoro-2-(methoxymethoxy)phenyl)-4,4,5,5-tetramethyl-1,3,2-dioxaborolane (CAS #2043962-01-0) (Intermediate KF)

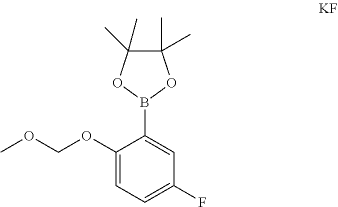 [SMILES: COCOc1ccc(F)cc1B1OC(C)(C)C(C)(C)O1]

### (4-Ethoxy-2-methoxyphenyl)boronic acid (CAS #1207443-48-8) (Intermediate KG)

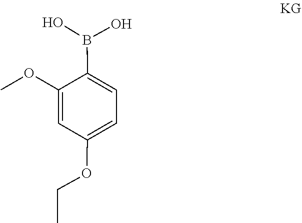

### 1-(3-(4-Cloro-7-fluoro-2-(4-(5-fluoro-3-methoxypyridin-2-yl)piperazine-1-carbonyl)-1H-indol-6-yl)-5,6-dihydropyridin-1(2H)-yl)-3-(1H-pyrazol-1-yl)propan-1-one (Intermediate KH)

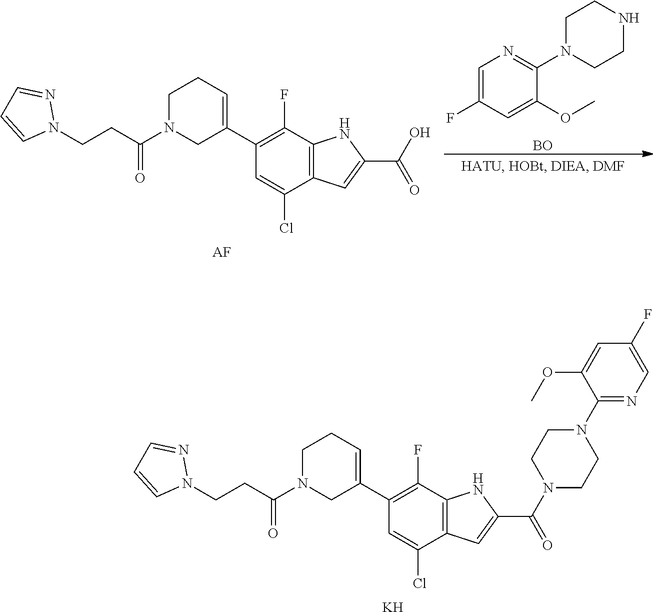

To a solution of 4-chloro-7-fluoro-6-[1-(3-pyrazol-1-ylpropanoyl)-3,6-dihydro-2H-pyridin-5-yl]-1H-indole-2-carboxylic acid (3.6 g, 8.64 mmol, Intermediate AF) in DMF (40 mL) was added HATU (3.94 g, 10.3 mmol), DIEA (4.46 g, 34.5 mmol) and HOBt (2.33 g, 17.2 mmol). Then 1-(5-fluoro-3-methoxy-2-pyridyl)piperazine (2.19 g, 10.3 mmol, Intermediate BO) was added at 0° C. and the mixture was stirred at 25° C. for 10 mins. On completion, the reaction mixture was poured into H₂O (80 mL) at 0° C., and then filtered and the filter cake was dried under reduced pressure to give a residue. The crude product was triturated with PE (60 mL) at 25° C. for 20 mins to afford the title compound (6 g, 93% yield) as a yellow solid. LC-MS (ESI⁺) m/z 610.1 (M+H)⁺; ¹H NMR (400 MHz, DMSO-d₆) δ=12.52 (d, J=6.0 Hz, 1H), 7.80 (d, J=2.4 Hz, 1H), 7.73-7.70 (m, 1H), 7.41 (d, J=14.8 Hz, 1H), 7.39-7.36 (m, 1H), 7.16-7.05 (m, 1H), 6.80 (s, 1H), 6.21-6.18 (m, 1H), 6.13 (d, J=9.2 Hz, 1H), 4.36 (q, J=6.4 Hz, 3H), 4.32-4.24 (m, 2H), 3.86 (s, 3H), 3.82 (s, 4H), 3.65-3.54 (m, 3H), 3.04-2.94 (m, 4H), 2.32 (d, J=1.6 Hz, 1H), 2.26 (s, 1H).

### 1-(3-(7-fluoro-2-(4-(5-fluoro-3-methoxypyridin-2-yl)piperazine-1-carbonyl)-4-(4,4,5,5-tetramethyl-1,3,2-dioxaborolan-2-yl)-1H-indol-6-yl)-5,6-dihydropyridin-1(2H)-yl)-3-(1H-pyrazol-1-yl)propan-1-one (Intermediate KI)

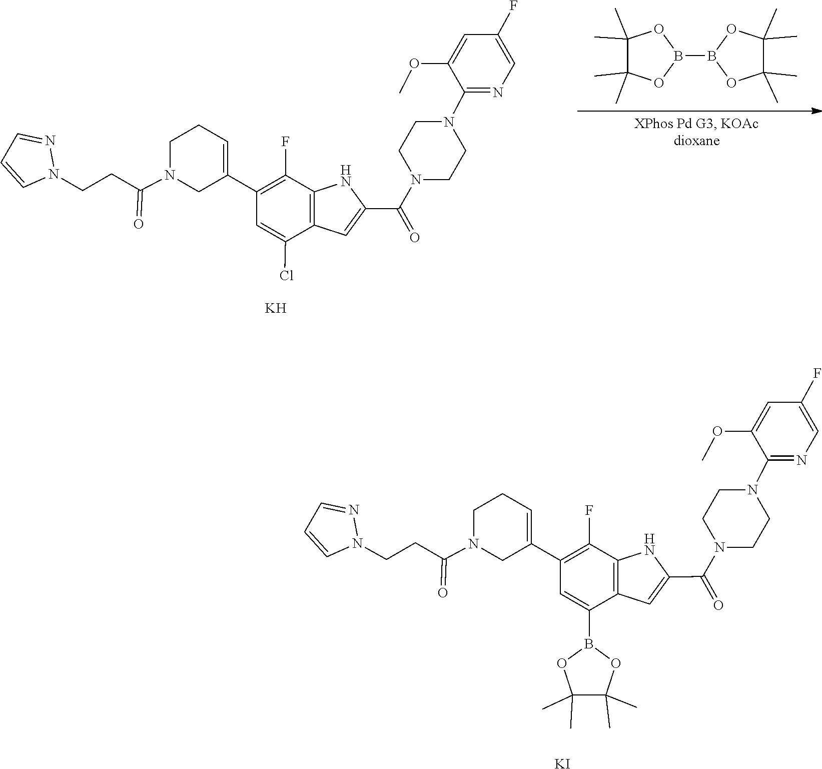

A mixture of 1-[5-[4-chloro-7-fluoro-2-[4-(5-fluoro-3-methoxy-2-pyridyl)piperazine-1-carbonyl]-1H-indol-6-yl]-3,6-dihydro-2H-pyridin-1-yl]-3-pyrazol-1-yl-propan-1-one (550 mg, 901 μmol, Intermediate KH), 4,4,5,5-tetramethyl-2-(4,4,5,5-tetramethyl-1,3,2-dioxaborolan-2-yl)-1,3,2-dioxaborolane (686 mg, 2.70 mmol, CAS #73183-34-3), KOAc (265 mg, 2.70 mmol) and XPhos Pd G3 (76.3 mg, 90.1 μmol) in dioxane (5 mL) was degassed and purged with N₂ three times. Then the mixture was stirred at 80° C. for 12 hrs under N₂ atmosphere. On completion, the reaction mixture was concentrated under reduced pressure to give a residue. The residue was purified by column chromatography (SiO₂, Dichloromethane. Methanol=50/1 to 10/1) to afford the title compound (480 mg, 68% yield) as a yellow solid. LC-MS (ESI⁺) m/z 702.3 (M+H)⁺; ¹H NMR (400 MHz, DMSO-d₆) δ=12.19-12.10 (m, 1H), 7.80 (d, J=2.4 Hz, 1H), 7.74-7.69 (m, 1H), 7.44-7.32 (m, 3H), 6.98 (s, 1H), 6.23-6.15 (m, 1H), 6.04 (s, 1H), 4.39-4.33 (m, 2H), 4.32-4.22 (m, 2H), 3.85 (s, 3H), 3.78 (s, 4H), 3.66-3.56 (m, 2H), 3.29 (d, J=2.4 Hz, 4H), 3.00-2.93 (m, 2H), 2.34-2.30 (m, 1H), 2.29-2.21 (m, 1H), 1.33 (s, 12H).

### 4-Chloro-3-ethyl-pyridine (CAS #860411-22-9) (Intermediate KJ)

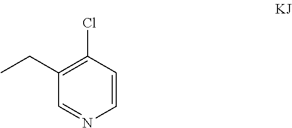

### 3-Bromo-4-isopropyl-pyridine (CAS #90731-96-7) (Intermediate KK)

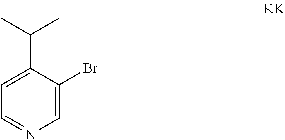

### Tert-butyl 4-(3-chloropyridazin-4-yl)piperazine-1-carboxylate (Intermediate KL)

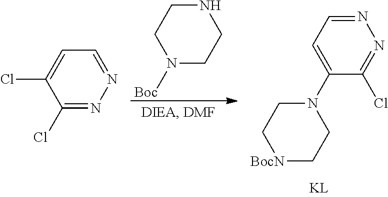

To a solution of 3,4-dichloropyridazine (0.9 g, 6.04 mmol) in DMF (15 mL) was added DIEA (2.34 g, 18.1 mmol, 3.16 mL) and tert-butyl piperazine-1-carboxylate (1.13 g, 6.04 mmol). Then the mixture was stirred at 60° C. for 12 hr. On completion, the reaction mixture was quenched by addition of H₂O (50 mL), and then diluted with EA (50 mL) and extracted with EA (100 mL×2). The combined organic layers were washed with NaCl (100 mL×2), dried over Na₂SO₄, filtered and concentrated under reduced pressure to give the title compound (1.9 g) as a white solid. ¹H NMR (400 MHz, DMSO-d6) δ=8.93 (d, J=5.6 Hz, 1H), 7.26 (d, J=5.6 Hz, 1H), 3.51-3.47 (m, 4H), 3.23-3.19 (m, 4H), 1.42 (s, 9H); LC-MS (ESI+) m/z 299.1 (M+H)⁺.

### 3-Methoxy-4-(piperazin-1-yl)pyridazine (Intermediate KM)

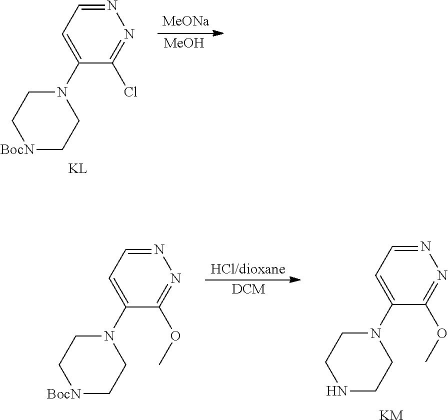

### Step 1—Tert-butyl 4-(3-methoxypyridazin-4-yl)piperazine-1-carboxylate

To a solution of tert-butyl 4-(3-chloropyridazin-4-yl)piperazine-1-carboxylate (1.8 g, 6.02 mmol, Intermediate KL) in MeOH (25 mL) was added NaOMe (5.4 M, 5.58 mL). The mixture was then stirred at 80° C. for 8 hrs. On completion, the reaction mixture was quenched by addition of H₂O (50 mL), and then diluted with EA (50 mL) and extracted with EA (100 mL×2). The combined organic layers were washed with NaCl (100 mL×2), dried over Na₂SO₄, filtered and concentrated under reduced pressure to give the title compound (1.7 g) as a white solid. ¹H NMR (400 MHz, DMSO-d6) δ=8.55 (d, J=5.3 Hz, 1H), 6.89 (d, J=5.4 Hz, 1H), 4.02 (s, 3H), 3.44 (br d, J=4.6 Hz, 4H), 3.23-3.19 (m, 4H), 1.41 (s, 9H); LC-MS (ESI+) m/z 295.1 (M+H)⁺.

### Step 2—3-Methoxy-4-(piperazin-1-yl)pyridazine

To a solution of tert-butyl 4-(3-methoxypyridazin-4-yl)piperazine-1-carboxylate (1 g, 3.40 mmol) in DCM (1 mL) was added HCl/dioxane (2 M, 10 mL). The mixture was then stirred at 25° C. for 0.5 hr. On completion, the reaction mixture was filtered and concentrated under reduced pressure to give the title compound (700 mg) as a white solid. LC-MS (ESI+) m/z 195.3 (M+H)⁺.

### 2-(4-Fluoro-2-(methoxymethoxy)phenyl)-4,4,5,5-tetramethyl-1,3,2-dioxaborolane (CAS #2284468-46-6) (Intermediate KN)

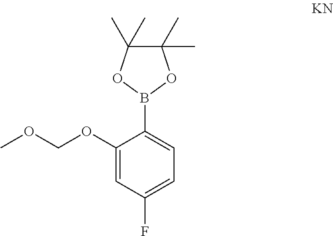

### 1-Phenyl-4-(4,4,5,5-tetramethyl-1,3,2-dioxaborolan-2-yl)-1H-pyrazole (CAS #1002334-12-4) (Intermediate KO)

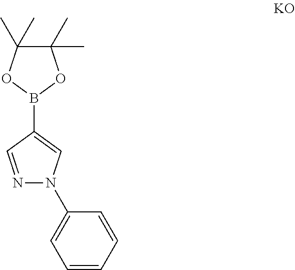 [SMILES: CC1(C)OB(c2cnn(-c3ccccc3)c2)OC1(C)C]

### (8-Methylquinolin-3-yl)boronic acid (CAS #1370040-72-4) (Intermediate KP)

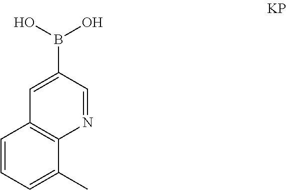

### 4,4,5,5-Tetramethyl-2-(m-tolyl)-1,3,2-dioxaborolane (CAS #253342-48-2) (Intermediate KQ)

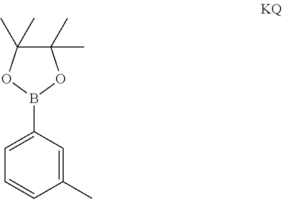

### 2-(3-Cyclopropylphenyl)-44,5,5-tetramethyl-1,3,2-dioxaborolane (CAS #627526-56-1) (Intermediate KR)

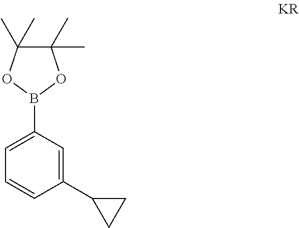 [SMILES: CC1(C)OB(c2cccc(C3CC3)c2)OC1(C)C]

### 4,4,5,5-tetramethyl-2-(prop-1-en-2-yl)-1,3,2-dioxaborolane (CAS #126726-62-3) (Intermediate KS)

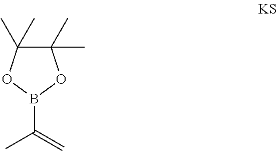

### 2-(4-fluoro-3-methylphenyl)-4,4,5,5-tetramethyl-1,3,2-dioxaborolane (CAS #1001200-60-7) (Intermediate KT)

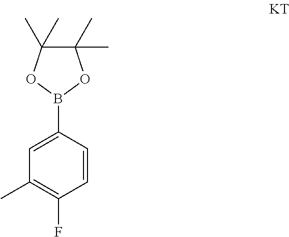

### (E)-4,4,5,5-tetramethyl-2-(pent-1-en-1-yl)-1,3,2-dioxaborolane (CAS #161395-96-6) (Intermediate KU)

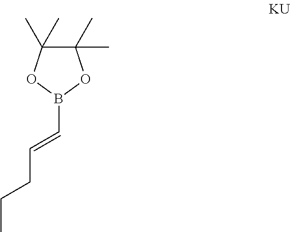

### (3,4-dihydro-2H-benzo[b][1,4]dioxepin-6-yl)boronic acid (CAS #1107064-09-4) (Intermediate KV)

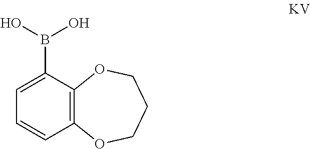 [SMILES: OB(O)c1cccc2c1OCCCO2]

### (2-(Ethylthio)phenyl)boronic acid (CAS #362045-33-8) (Intermediate KW)

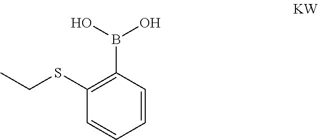

### (2-morpholinopyridin-4-yl)boronic acid (CAS #888721-86-6)(Intermediate KX)

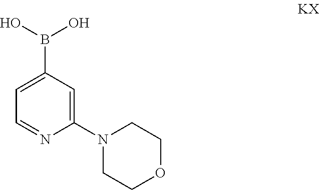 [SMILES: OB(O)c1ccnc(N2CCOCC2)c1]

### 1-(2-Fluoro-3-(4,4,5,5-tetramethyl-1,3,2-dioxaborolan-2-yl)phenyl)cyclopropane-1-carbonitrile (Intermediate KY)

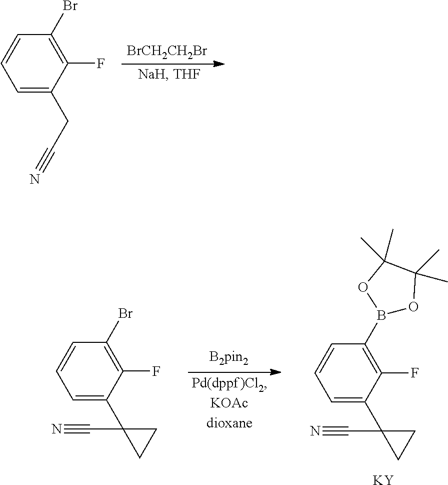

### Step 1—1-(2-Fluoro-3-methylphenyl)cyclopropane-1-carbonitrile

To a solution of 2-(3-bromo-2-fluoro-phenyl)acetonitrile (3 g, 14 mmol, CAS #874285-03-7) in THF (40 mL) was added NaH (2.80 g, 70.1 mmol, 60% dispersion in mineral oil) at 0° C. The reaction was stirred at 0° C. for 1 hr. Then 1,2-dibromoethane (3.16 g, 16.8 mmol, 1.27 mL, CAS #106-93-4) was added into the mixture and the reaction was stirred at 25° C. for 1 hr. On completion, the reaction mixture was quenched by NH₄Cl until the pH=7 and extracted with ethyl acetate (3×100 mL). The combined organic layers were washed with brine (200 mL) and dried over anhydrous sodium sulfate, filtered and the filtrate was concentrated in vacuo to give the crude residue. The residue was purified by column chromatography (SiO₂, Petroleum ether/Ethyl acetate=1/0 to 10/1) to give the title compound (2.82 g, 84% yield) as a yellow solid. LC-MS (ESI⁺) m/z 239.9 (M+H)⁺; ¹H NMR (400 MHz, DMSO-d₆) δ=1.32-1.55 (m, 2H) 1.68-1.81 (m, 2H) 7.18 (td, J=7.6, 0.8 Hz, 1H) 7.34-7.60 (m, 1H) 7.73 (ddd, J=8.4, 6.8, 1.6 Hz, 1H).

### Step 2—1-(2-Fluoro-3-(4,4,5,5-tetramethyl-1,3,2-dioxaborolan-2-yl)phenyl)cyclopropane-1-carbonitrile

A mixture of 1-(3-bromo-2-fluoro-phenyl)cyclopropanecarbonitrile (300 mg, 1.25 mmol), 4,4,5,5-tetramethyl-2-(4,4,5,5-tetramethyl-1,3,2-dioxaborolan-2-yl)-1,3,2-dioxaborolane (634 mg, 2.50 mmol, CAS #73183-34-3), Pd(dppf)Cl₂·CH₂Cl₂ (153 mg, 188 μmol), and KOAc (368 mg, 3.75 mmol) in dioxane (4 mL) was degassed and purged with N₂ three times. Then the mixture was stirred at 80° C. for 2 hrs under N₂ atmosphere. On completion, the reaction mixture was filtered and the filtrate was concentrated in vacuo to give the crude residue. The crude product was purified by column chromatography (SiO₂, Petroleum ether/Ethyl acetate=0/1 to 10/1) then the residue was purified by prep-HPLC (0.1% FA condition) to give the title compound (96 mg, 27% yield) was a white solid. LC-MS (ESI⁺) m/z 310.1 (M+H)⁺.

### 2-Methyl-2-(3-(4,4,5,5-tetramethyl-1,3,2-dioxaborolan-2-yl)phenyl)propanenitrile (CAS #1160502-10-2) (Intermediate KZ)

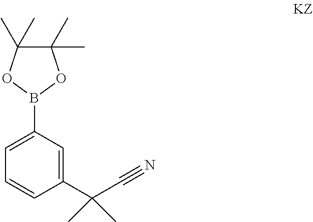 [SMILES: CC(C)(C#N)c1cccc(B2OC(C)(C)C(C)(C)O2)c1]

### 6-(1-(3-(1H-1,2,3-triazol-1-yl)propanoyl)-1,2,5,6-tetrahydropyridin-3-yl)-4-(3-ethylpyridin-4-yl)-7-fluoro-1H-indole-2-carboxylic acid (Intermediate LA)

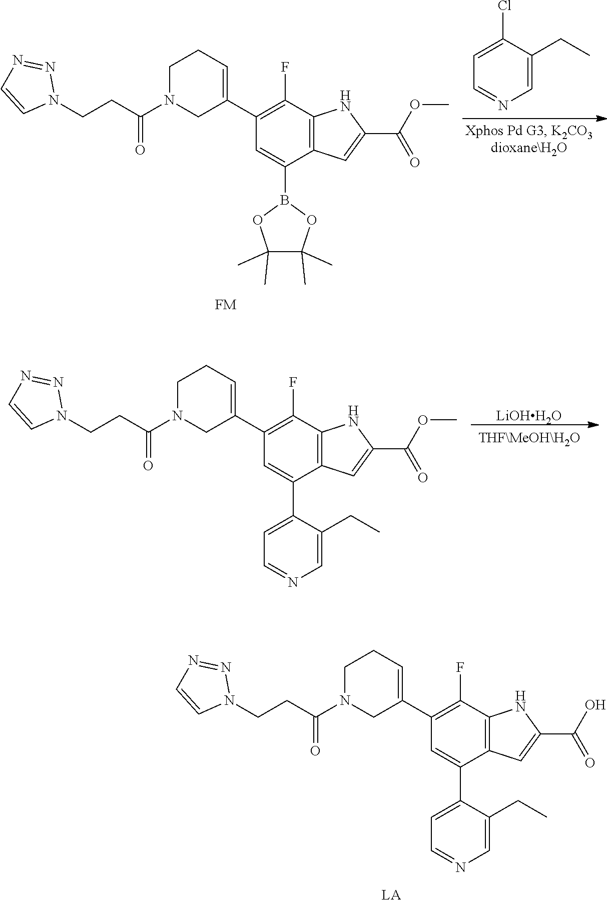

### Step 1—Methyl 6-(1-(3-(1H-1,2,3-triazol-1-yl)propanoyl)-1,2,5,6-tetrahydropyridin-3-yl)-4-(3-ethylpyridin-4-yl)-7-fluoro-1H-indole-2-carboxylate

To a solution of methyl 7-fluoro-4-(4,4,5,5-tetramethyl-1,3,2-dioxaborolan-2-yl)-6-[1-[3-(triazol-1-yl)propanoyl]-3,6-dihydro-2H-pyridin-5-yl]-1H-indole-2-carboxylate (92.4 mg, 176 μmol, Intermediate FM), 4-chloro-3-ethyl-pyridine (75 mg, 529 μmol) in dioxane (1.5 mL) and H₂O (0.5 mL) was added XPhos Pd G3 (14.9 mg, 17.7 μmol) and K₂CO₃ (73.20 mg, 529 μmol). The mixture was stirred at 80° C. for 2 hr. On completion, the residue was purified by column chromatography (SiO₂, Petroleum ether/Ethyl acetate=10/1 to 1/2) to give the title compound (100 mg, 87% yield) as a white solid. LC-MS (ESI+) m/z 503.2 (M+H)⁺.

### Step 2—6-(1-(3-(1H-1,2,3-triazol-1-yl)propanoyl)-1,2,5,6-tetrahydropyridin-3-yl)-4-(3-ethylpyridin-4-yl)-7-fluoro-1H-indole-2-carboxylic acid

To a solution of methyl 4-(3-ethyl-4-pyridyl)-7-fluoro-6-[1-[3-(triazol-1-yl)propanoyl]-3,6-dihydro-2H-pyridin-5-yl]-1H-indole-2-carboxylate (0.1 g, 159 μmol) in MeOH (0.5 mL), THF (0.5 mL) and H₂O (0.5 mL) was added LiOH·H₂O (33.4 mg, 796 μmol). The mixture was then stirred at 40° C. for 1 hr. On completion, the mixture was purified by reversed-phase HPLC (0.1% FA condition) to give the title compound (50 mg, 57% yield, FA) as a white solid. LC-MS (ESI+) m/z 489.2 (M+H)⁺.

### 1-(3-(4-Chloro-2-(4-(3,5-dimethoxypyridin-2-yl)piperazine-1-carbonyl)-7-fluoro-1H-indol-6-yl)-5,6-dihydropyridin-1(2H)-yl)-3-(1H-pyrazol-1-yl)propan-1-one (Intermediate LB)

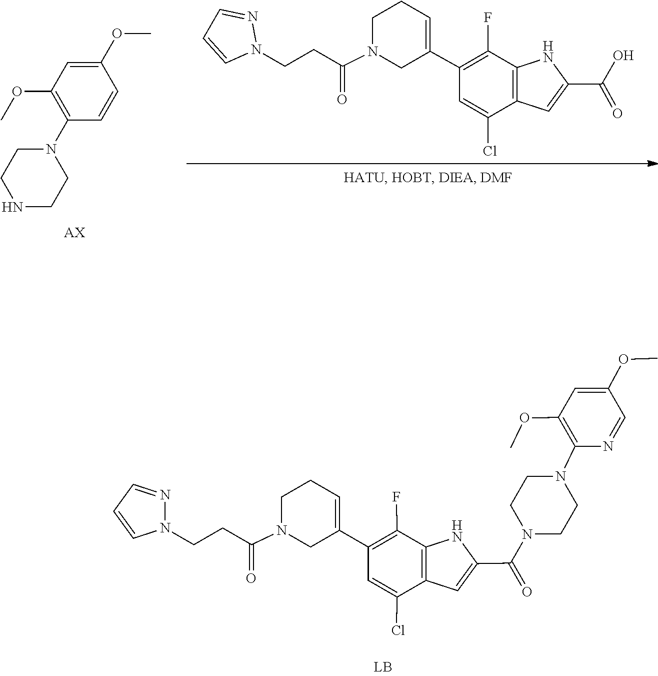

To a solution of 4-chloro-7-fluoro-6-[1-(3-pyrazol-1-ylpropanoyl)-3,6-dihydro-2H-pyridin-5-yl]-1H-indole-2-carboxylic acid (1.4 g, 3.36 mmol, Intermediate AF) in DMF (12 mL) was added HATU (1.53 g, 4.03 mmol), DIEA (1.30 g, 10.08 mmol) and HOBt (907 mg, 6.72 mmol). Then 1-(3,5-dimethoxy-2-pyridyl)piperazine (785 mg, 3.02 mmol, HCl, Intermediate AX) was added and the mixture was stirred at 25° C. for 0.5 hrs. On completion, the reaction mixture was filtered and concentrated under reduced pressure to give a residue. The residue was purified by prep-HPLC (HCl condition) to give the title compound (1.17 g, 48% yield, HCl) as a white solid. LC-MS (ESI⁺) m/z 622.6 (M+H)⁺.

### 1-(5-(2-(4-(3,5-Dimethoxypyridin-2-yl)piperazine-1-carbonyl)-7-fluoro-4-(4,4,5,5-tetramethyl-1,3,2-dioxaborolan-2-yl)-1H-indol-6-yl)-3,6-dihydropyridin-1(2H)-yl)-3-(1H-pyrazol-1-yl)propan-1-one (Intermediate LC)

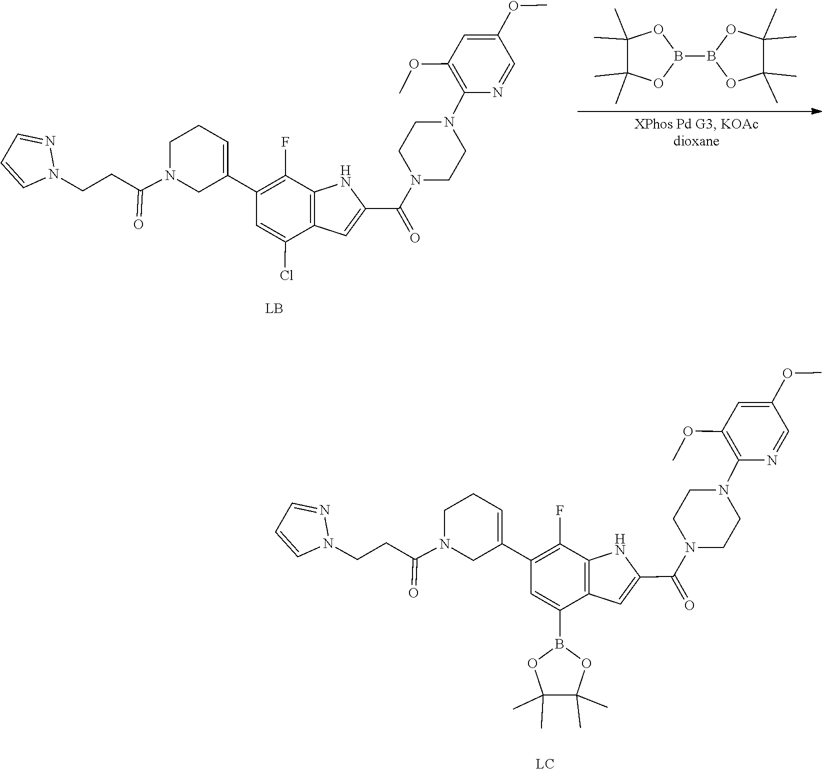

To a solution of 1-[5-[4-chloro-2-[4-(3,5-dimethoxy-2-pyridyl)piperazine-1-carbonyl]-7-fluoro-1H-indol-6-yl]-3,6-dihydro-2H-pyridin-1-yl]-3-pyrazol-1-yl-propan-1-one (1.1 g, 1.8 mmol, Intermediate LB) and 4,4,5,5-tetramethyl-2-(4,4,5,5-tetramethyl-1,3,2-dioxaborolan-2-yl)-1,3,2-dioxaborolane (1.80 g, 7.07 mmol, CAS #73183-34-3) in dioxane (8 mL) was added XPhos Pd G3 (149 mg, 176 μmol) and KOAc (520 mg, 5.30 mmol). The mixture was stirred at 90° C. for 2 hrs under N₂ atmosphere. On completion, the reaction mixture was filtered and concentrated under reduced pressure to give the title compound (0.56 g, 38% yield) as a brown solid. LC-MS (ESI⁺) m/z 714.3 (M+H)⁺; ¹H NMR (400 MHz, DMSO-d6) δ=12.17 (s, 1H), 7.96 (s, 2H), 7.76-7.69 (m, 1H), 7.56 (s, 1H), 7.46-7.38 (m, 1H), 7.35 (d, J=6.4 Hz, 1H), 6.99 (s, 2H), 6.23-6.17 (m, 1H), 6.05 (s, 1H), 4.41-4.26 (m, 4H), 3.81 (d, J=15.6 Hz, 10H), 3.58 (t, J=5.2 Hz, 2H), 3.21 (s, 4H), 3.01-2.92 (m, 2H), 2.90 (s, 5H), 2.74 (d, J=0.4 Hz, 4H), 1.34 (s, 9H).

### 4-Chloro-3-isopropylpyridine (Intermediate LD)

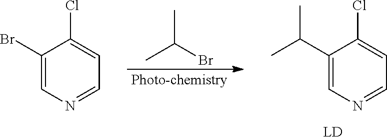

To a solution of 3-bromo-4-chloro-pyridine (1 g, 5 mmol) 2-bromopropane (767 mg, 6.24 mmol, 585 μL) and 2-bromopropane (767 mg, 6.24 mmol, 585 μL) in DME (20 mL) was added bis(trimethylsilyl)silyl-trimethyl-silane (1.42 g, 5.72 mmol, 1.76 mL), bis[3,5-difluoro-2-[5-(trifluoromethyl)-2-pyridyl]phenyl]iridium(1+);4-tert-butyl-2-(4-tert-butyl-2-pyridyl)pyridine;hexafluorophosphate (58.3 mg, 51.9 μmol), 2,6-dimethylpyridine (1.11 g, 10.4 mmol, 1.21 mL) and 4-tert-butyl-2-(4-tert-butyl-2-pyridyl)pyridine dichloronickel (31.0 mg, 77.9 μmol). The mixture was then stirred at 25° C. for 12 hrs. On completion, the crude product was purified by reversed-phase HPLC(0.1% NH₃·H₂O) to give the title compound (200 mg, 20% yield) as a white solid. LC-MS (ESI+) m/z 156.0 (M+H)⁺.

### 5-Piperazin-1-yl-1H-pyrrolo[2,3-b]pyridine-6-carbonitrile (Intermediate LE)

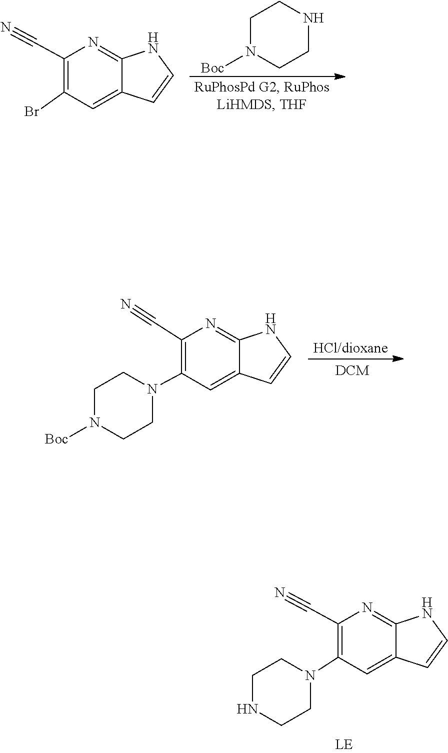 [SMILES: N#Cc1nc2[nH]ccc2cc1Br ; CC(C)(C)OC(=O)N1CCNCC1 ; CC(C)(C)OC(=O)N1CCN(c2cc3cc[nH]c3nc2C#N)CC1 ; N#Cc1nc2[nH]ccc2cc1N1CCNCC1]

### Step 1—Tert-butyl 4-(6-cyano-1H-pyrrolo[2,3-b]pyridin-5-yl)piperazine-1-carboxylate

A mixture of 5-bromo-1H-pyrrolo[2,3-b]pyridine-6-carbonitrile (400 mg, 1.80 mmol, CAS #1190317-45-3), tert-butyl piperazine-1-carboxylate (503.29 mg, 2.70 mmol, CAS #57260-71-6), RuPhos (84.06 mg, 180.15 μmol), and Ruphos Pd G2 (139.92 mg, 180.15 μmol) in THF (10 mL) was degassed and purged with N₂ three times. Then to the mixture was added LiHMDS (1 M, 5.40 mmol) and the stirred at 60° C. for 6 hrs under N₂ atmosphere. On completion, the reaction was quenched with saturated NH₄Cl (20 mL) and the mixture was extracted with ethyl acetate (3×20 mL). The combined organic extracts were washed with brine (3×20 mL) and then dried over anhydrous sodium sulfate, filtered and concentrated under vacuum to give a residue. The residue was purified by flash silica gel chromatography (ISCO®; 12 g Sepa Flash® Silica Flash Column, Eluent of 0˜30% Ethyl acetate/Petroleum ether gradient @80 mL/min) to give the title compound (220 mg, 37% yield) as a yellow solid. ¹H NMR (400 MHz, DMSO-d₆) δ=12.05 (s, 1H), 7.92 (s, 1H), 7.79 (t, J=2.8 Hz, 1H), 6.52 (dd, J=1.6, 3.2 Hz, 1H), 3.52 (s, 4H), 3.07-2.95 (m, 4H), 1.43 (s, 9H).

### Step 2—5-Piperazin-1-yl-1H-pyrrolo[2,3-b]pyridine-6-carbonitrile

To a solution of tert-butyl 4-(6-cyano-1H-pyrrolo[2,3-b]pyridin-5-yl)piperazine-1-carboxylate (220 mg, 672.00 μmol) in DCM (3 mL) was added HCl/dioxane (2 M, 10 mL). The mixture was stirred at 20° C. for 3 hrs. On completion, the mixture was concentrated under vacuum to give the title compound (200 mg, HCl salt) as yellow solid. LC-MS (ESI+) m/z 228.1 (M+H)⁺.

### 2,2-Difluoro-4-iodo-1,3-benzodioxole (CAS #531508-54-0) (Intermediate LF)

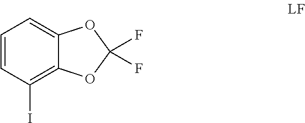 [SMILES: FC1(F)Oc2cccc(I)c2O1]

### Methyl 6-(1-(3-(1H-pyrazol-1-yl)propanoyl)-1,2,5,6-tetrahydropyridin-3-yl)-7-fluoro-4-(4,4,5,5-tetramethyl-1,3,2-dioxaborolan-2-yl)-1H-indole-2-carboxylate (Intermediate LG)

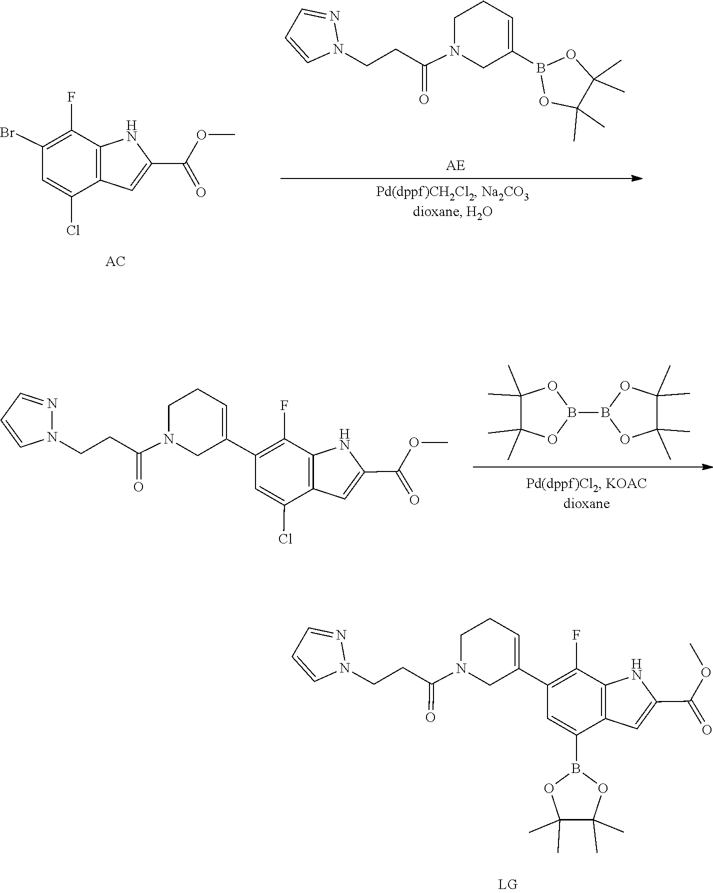

### Step 1—Methyl 6-(1-(3-(1H-pyrazol-1-yl)propanoyl)-1,2,5,6-tetrahydropyridin-3-yl)-4-chloro-7-fluoro-1H-indole-2-carboxylate

A mixture of methyl 6-bromo-4-chloro-7-fluoro-1H-indole-2-carboxylate (10 g, 32.6 mmol, Intermediate AC), 3-pyrazol-1-yl-1-[5-(4,4,5,5-tetramethyl-1,3,2-dioxaborolan-2-yl)-3,6-dihydro-2H-pyridin-1-yl]propan-1-one (16.2 g, 48.9 mmol, Intermediate AE), Pd(dppf)Cl₂ CH₂Cl₂ (2.66 g, 3.26 mmol) and Na₂CO₃ (10.3 g, 97.8 mmol) in dioxane (210 mL) and H₂O (35 mL) was degassed and purged with N₂ three times. Then the mixture was stirred at 80° C. for 12 hrs under N₂ atmosphere. The crude product was purified directly by trituration with H₂O (1000 mL) at 0° C. for 30 minutes, followed by filtration and drying to afford the title compound (17 g, 96% yield) as a yellow solid. LC-MS (ESI⁺) m/z 431.1 (M+H)⁺; ¹H NMR (400 MHz, DMSO-d₆) δ=12.90 (d, J=4.4 Hz, 1H), 7.71 (dd, J=2.0, 7.6 Hz, 1H), 7.63-7.45 (m, 1H), 7.41 (d, J=17.6 Hz, 1H), 7.23-7.07 (m, 2H), 6.25-6.10 (m, 2H), 4.36 (q, J=7.2 Hz, 2H), 4.32-4.23 (m, 2H), 3.90 (s, 3H), 3.62 (t, J=5.6 Hz, 1H), 2.97 (q, J=7.2 Hz, 2H), 2.36-2.22 (m, 2H).

### Step 2—Methyl 6-(1-(3-(1H-pyrazol-1-yl)propanoyl)-1,2,5,6-tetrahydropyridin-3-yl)-7-fluoro-4-(4,4,5,5-tetramethyl-1,3,2-dioxaborolan-2-yl)-1H-indole-2-carboxylate

A mixture of methyl 6-(1-(3-(1H-pyrazol-1-yl)propanoyl)-1,2,5,6-tetrahydropyridin-3-yl)-4-chloro-7-fluoro-1H-indole-2-carboxylate (900 mg, 2.09 mmol), 4,4,5,5-tetramethyl-2-(4,4,5,5-tetramethyl-1,3,2-dioxaborolan-2-yl)-1,3,2-dioxaborolane (1.59 g, 6.27 mmol), KOAc (615 mg, 6.27 mmol) and XPhos Pd G3 (177 mg, 209 μmol) in dioxane (10.0 mL) was degassed and purged with N₂ three times. Then the mixture was stirred at 80° C. for 2 hrs under N₂ atmosphere. On completion, the reaction mixture was filtered and concentrated under reduced pressure to give a residue. The crude product was purified by MPLC (SiO₂, PE:EA=10:1 to 0:1) to give the title compound (880 mg, 69% yield) as a yellow solid. LC-MS (ESI⁺) m/z 523.2 (M+H)⁺; ¹H NMR (400 MHz, CHLOROFORM-d) δ=9.07 (d, J=7.2 Hz, 1H), 7.70-7.62 (m, 1H), 7.53-7.51 (m, 1H), 7.50-7.47 (m, 1H), 6.23 (d, J=10.4 Hz, 1H), 6.12 (d, J=17.6 Hz, 1H), 4.58-4.52 (m, 2H), 4.45 (s, 1H), 4.22 (s, 1H), 4.00-3.97 (m, 3H), 3.76 (t, J=6.0 Hz, 1H), 3.54 (t, J=5.6 Hz, 1H), 3.04-2.96 (m, 2H), 2.33 (s, 2H), 1.40 (d, J=4.0 Hz, 12H).

### 6-(1-(3-(1H-pyrazol-1-yl)propanoyl)-1,2,5,6-tetrahydropyridin-3-yl)-4-(4-ethoxypyridin-3-yl)-7-fluoro-1H-indole-2-carboxylic acid (Intermediate LH)

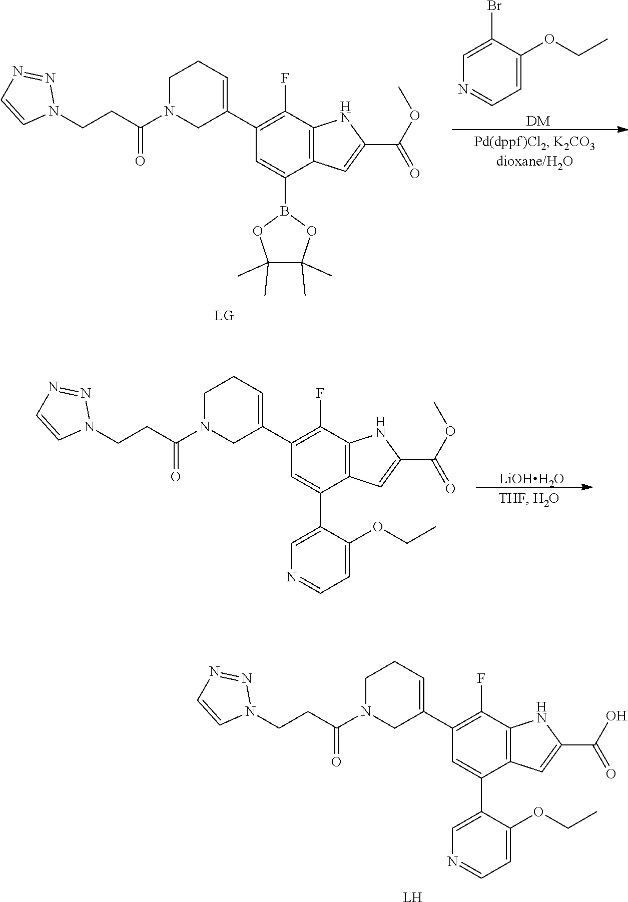

### Step 1-Methyl 6-(1-(3-(1H-pyrazol-1-yl)propanoyl)-1,2,5,6-tetrahydropyridin-3-yl)-4-(4-ethoxypyridin-3-yl)-7-fluoro-1H-indole-2-carboxylate

A mixture of methyl 7-fluoro-6-[1-(3-pyrazol-1-ylpropanoyl)-3,6-dihydro-2H-pyridin-5-yl]-4-(4,4,5,5-tetramethyl-1,3,2-dioxaborolan-2-yl)-1H-indole-2-carboxylate (880 mg, 1.68 mmol, Intermediate LG), 3-bromo-4-ethoxy-pyridine (340 mg, 1.68 mmol, Intermediate DM), Pd(dppf)Cl₂·CH₂Cl₂ (138 mg, 168 μmol), and K₂CO₃ (698 mg, 5.05 mmol) in dioxane (5.00 mL) and H₂O (1.00 mL) was degassed and purged with N₂ three times. Then the mixture was stirred at 80° C. for 12 hrs under N₂ atmosphere. On completion, the reaction mixture was filtered and concentrated under reduced pressure to give a residue. The crude product was purified by MPLC (SiO₂, PE:EA=10:1 to 0:1) to give the title compound (680 mg, 73% yield) as a yellow oil. LC-MS (ESI⁺) m/z 518.2 (M+H)⁺.

### Step 2—6-(1-(3-(1H-pyrazol-1-yl)propanoyl)-1,2,5,6-tetrahydropyridin-3-yl)-4-(4-ethoxypyridin-3-yl)-7-fluoro-1H-indole-2-carboxylic acid

To a solution of methyl 4-(4-ethoxy-3-pyridyl)-7-fluoro-6-[1-(3-pyrazol-1-ylpropanoyl)-3,6-dihydro-2H-pyridin-5-yl]-1H-indole-2-carboxylate (680 mg, 1.31 mmol) in THF (4.00 mL) and H₂O (2.00 mL) was added LiOH·H₂O (276 mg, 6.57 mmol). Then the mixture was stirred at 25° C. for 12 hrs. On completion, to the reaction mixture was added HCl (2M) (10.0 mL) and solid formed, and was filtered to give the title compound (600 mg) as a white solid. LC-MS (ESI⁺) m/z 504.2 (M+H)⁺; ¹H NMR (400 MHz, DMSO-d₆) δ=12.46-12.36 (m, 1H), 8.57-8.45 (m, 2H), 7.75-7.68 (m, 1H), 7.45-7.35 (m, 1H), 7.32-7.25 (m, 1H), 7.07 (dd, J=6.0, 17.6 Hz, 1H), 6.87 (s, 1H), 6.18 (d, J=13.6 Hz, 2H), 4.37-4.34 (m, 2H), 4.24-4.17 (m, 2H), 3.64 (t, J=5.6 Hz, 2H), 3.57 (br t, J=5.6 Hz, 2H), 3.00-2.94 (m, 2H), 2.36-2.24 (m, 2H), 1.20 (td, J=6.8, 10.6 Hz, 3H).

### (R)-6-(1-(3-(1H-pyrazol-1-yl)propanoyl)piperidin-3-yl)-7-fluoro-4-(4-methoxypyridin-3-yl)-1H-indole-2-carboxylic acid (Intermediate LI)

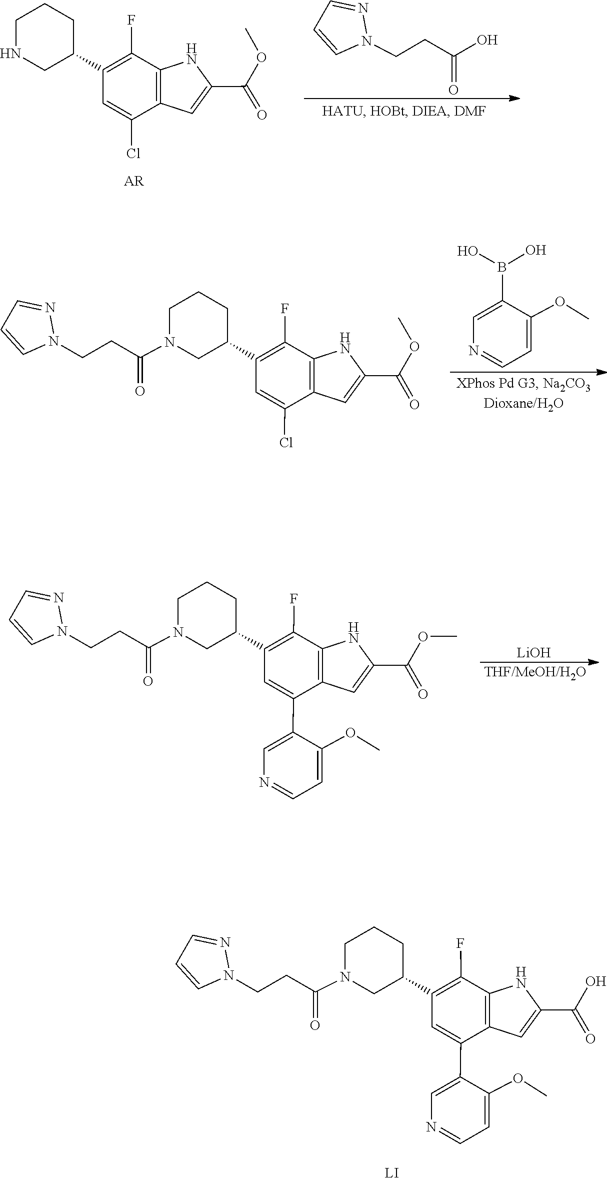

### Step 1—Methyl (R)-6-(1-(3-(1H-pyrazol-1-yl)propanoyl)piperidin-3-yl)-4-chloro-7-fluoro-1H-indole-2-carboxylate

To a solution of methyl 4-chloro-7-fluoro-6-[(3R)-3-piperidyl]-1H-indole-2-carboxylate (0.5 g, 1.61 mmol, Intermediate AR) and 3-pyrazol-1-ylpropanoic acid (225 mg, 1.61 mmol, 1 eq) in DMF (5 mL) was added HATU (795 mg, 2.09 mmol), HOBt (283 mg, 2.09 mmol) and DIEA (1.04 g, 8.05 mmol, 1.40 mL). The mixture was then stirred at 25° C. for 0.2 hr. On completion, the reaction mixture was quenched by addition of H₂O (20 mL), and then diluted with EA (10 mL) and extracted with EA (30 mL×2). The combined organic layers were washed with sat. NaCl solution (20 mL×3), dried over Na₂SO₄, filtered and concentrated under reduced pressure to give a residue. The residue was purified by column chromatography (SiO₂, Petroleum ether/Ethyl acetate=10/1 to 1/1) to give the title compound (0.5 g, 65% yield) as a white solid. LC-MS (ESI+) m/z 433.1 (M+H)⁺.

### Step 2—Methyl (R)-6-(1-(3-(1H-pyrazol-1-yl)propanoyl)piperidin-3-yl)-7-fluoro-4-(4-methoxypyridin-3-yl)-1H-indole-2-carboxylate

To a solution of methyl 4-chloro-7-fluoro-6-[(3R)-1-(3-pyrazol-1-ylpropanoyl)-3-piperidyl]-1H-indole-2-carboxylate (0.5 g, 1.04 mmol) and (4-methoxy-3-pyridyl)boronic acid (159 mg, 1.04 mmol) in dioxane (6 mL) and H₂O (2 mL) was added XPhos Pd G3 (88 mg, 104 μmol) and Na₂CO₃ (330 mg, 3.12 mmol). The mixture was then stirred at 80° C. for 2 hr. On completion, the reaction mixture was quenched by addition of H₂O (10 mL), and then diluted with EA (20 mL) and extracted with EA (30 mL×2). The combined organic layers were washed with sat. NaCl solution (20 mL×2), dried over Na₂SO₄, filtered and concentrated under reduced pressure to give a residue. The residue was purified by column chromatography (SiO₂, Petroleum ether/Ethyl acetate=10/1 to 1/2) to give the title compound (450 mg, 67% yield) as a white solid. LC-MS (ESI+) m/z 506.5 (M+H)⁺.

### Step 3—(R)-6-(1-(3-(1H-pyrazol-1-yl)propanoyl)piperidin-3-yl)-7-fluoro-4-(4-methoxypyridin-3-yl)-1H-indole-2-carboxylic acid

To a solution of methyl 7-fluoro-4-(4-methoxy-3-pyridyl)-6-[(3R)-1-(3-pyrazol-1-ylpropanoyl)-3-piperidyl]-1H-indole-2-carboxylate (450 mg, 694 μmol) in THF (2 mL), MeOH (2 mL) and H₂O (2 mL) was added LiOH·H₂O (87.4 mg, 2.08 mmol). The mixture was the stirred at 40° C. for 1 hr. On completion, the crude product was purified by reversed-phase HPLC(0.1% FA condition) to give the title compound (280 mg, 75% yield, FA) as a white solid. LC-MS (ESI+) m/z 492.3 (M+H)⁺.

### (R)-6-(1-(3-(1H-1,2,3-triazol-1-yl)propanoyl)piperidin-3-yl)-7-fluoro-4-(4-methoxypyridin-3-yl)-1H-indole-2-carboxylic acid (Intermediate LJ)

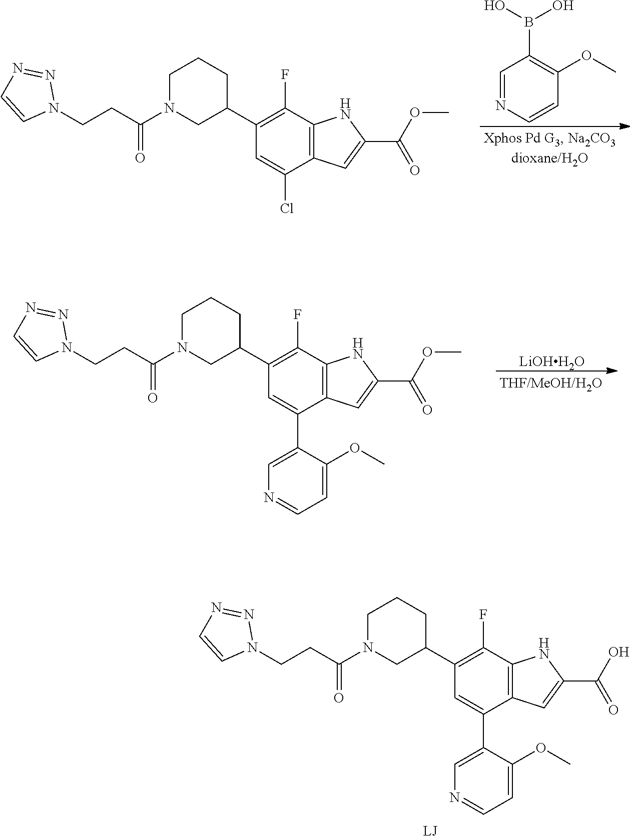

### Step 1—(R)-methyl 6-(1-(3-(1H-1,2,3-triazol-1-yl)propanoyl)piperidin-3-yl)-7-fluoro-4-(4-methoxypyridin-3-yl)-1H-indole-2-carboxylate

A mixture of (R)-methyl 4-chloro-7-fluoro-6-(piperidin-3-yl)-1H-indole-2-carboxylate (850 mg, 1.96 mmol, synthesized via Step 1 of Intermediate AS), (4-methoxypyridin-3-yl)boronic acid (449 mg, 2.94 mmol, CAS #355004-67-0), Na₂CO₃ (623 mg, 5.88 mmol), and XPhos Pd G3 (166 mg, 196 μmol) in dioxane (15 mL) and H₂O (3 mL) was degassed and purged with N₂ three times. Then the mixture was stirred at 80° C. for 12 hrs under N₂ atmosphere. On completion, the reaction mixture was concentrated in vacuo to give the crude residue. The residue was purified by column chromatography (SiO₂, Petroleum ether:Ethyl acetate=1/1 to 0/1) (SiO₂, Dichloromethane:Methanol=1/0 to 10/1) to give a title compound (750 mg, 69% yield) as a yellow solid. LC-MS (ESI⁺) m/z 507.2 (M+H)⁺.

### Step 2—(R)-6-(1-(3-(1H-1,2,3-triazol-1-yl)propanoyl)piperidin-3-yl)-7-fluoro-4-(4-methoxypyridin-3-yl)-1H-indole-2-carboxylic acid

To a solution of (R)-methyl 6-(1-(3-(1H-1,2,3-triazol-1-yl)propanoyl)piperidin-3-yl)-7-fluoro-4-(4-methoxypyridin-3-yl)-1H-indole-2-carboxylate (400 mg, 790 μmol) in THF (2 mL), MeOH (2 mL) and H₂O (2 mL) was added LiOH·H₂O (331 mg, 7.90 mmol). The mixture was stirred at 40° C. for 0.5 hrs. On completion, HCl (12 M) was added to the mixture until the pH was 6.0 and filtered. The filter cake was dried in vacuo to give the title compound (320 mg) as a yellow solid. LC-MS (ESI⁺) m/z 493.1 (M+H)⁺.

### 7-Fluoro-6-methoxy-4-(4,4,5,5-tetramethyl-1,3,2-dioxaborolan-2-yl)quinoline (CAS #2248157-24-4) (Intermediate LK)

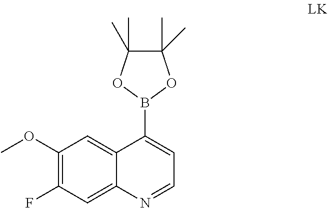

### 2-Bromo-6-(difluoromethoxy)-3-methoxypyridine (Intermediate LL)

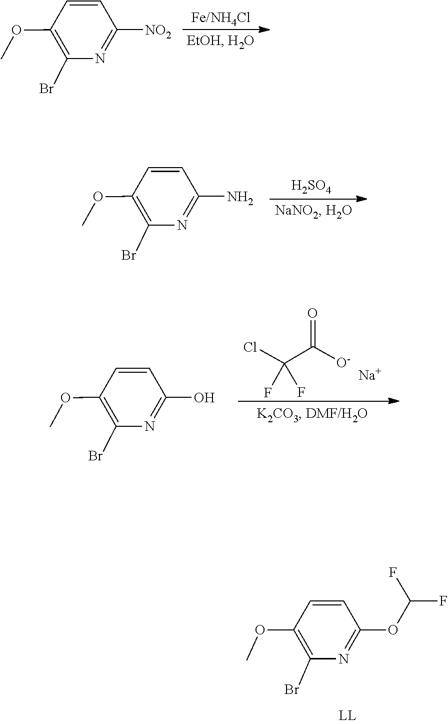

### Step 1—6-Bromo-5-methoxypyridin-2-amine

A mixture of 2-bromo-3-methoxy-6-nitropyridine (5 g, 20 mmol, CAS #76066-07-4), NH₄Cl (11.4 g, 214 mmol) in EtOH (50 mL) and H₂O (50 mL) was added Fe (11.9 g, 214 mmol) and purged with N₂ three times. Then the mixture was stirred at 25° C. for 12 hrs under N₂ atmosphere. On completion, the reaction mixture was concentrated under reduced pressure to give the title compound (4.3 g) as a black solid. LC-MS (ESI⁺) m/z 202.9 (M+H)⁺.

### Step 2—6-Bromo-5-methoxypyridin-2-ol

A mixture of 6-bromo-5-methoxypyridin-2-amine (2 g, 10 mmol) in H₂O (18.4 mL) and H₂SO₄ (1.6 mL) was added a mixture of NaNO₂ (815 mg, 11.8 mmol) in H₂O (1.6 mL) and then the mixture was stirred at 0° C. for 2 hrs. On completion, the reaction mixture was filtered and concentrated under reduced pressure to give the title compound (1 g) as an off-white solid. LC-MS (ESI⁺) m/z 203.9 (M+H)⁺.

### Step 3—2-Bromo-6-(difluoromethoxy)-3-methoxypyridine

To a solution of 6-bromo-5-methoxypyridin-2-ol (1 g, 4.90 mmol) in DMF (10 mL) and H₂O (1 mL) was added K₂CO₃ (1.02 g, 7.35 mmol) and sodium 2-chloro-2,2-difluoroacetate (3.74 g, 24.5 mmol, CAS #1895-39-2). The mixture was then stirred at 100° C. for 12 hrs. On completion, the reaction mixture was filtered and concentrated under reduced pressure to give a residue. The crude residue was purified by reversed-phase HPLC (FA condition) to give the title compound (400 mg, 26% yield, FA) as a white solid. ¹H NMR (400 MHz, DMSO-d₆) δ=7.71 (d, J=8.8 Hz, 1H), 7.52-7.33 (m, 1H), 7.16 (d, J=8.4 Hz, 1H), 3.89 (s, 3H).

### 1-(6-(Difluoromethoxy)-3-methoxypyridin-2-yl)piperazine (Intermediate LM)

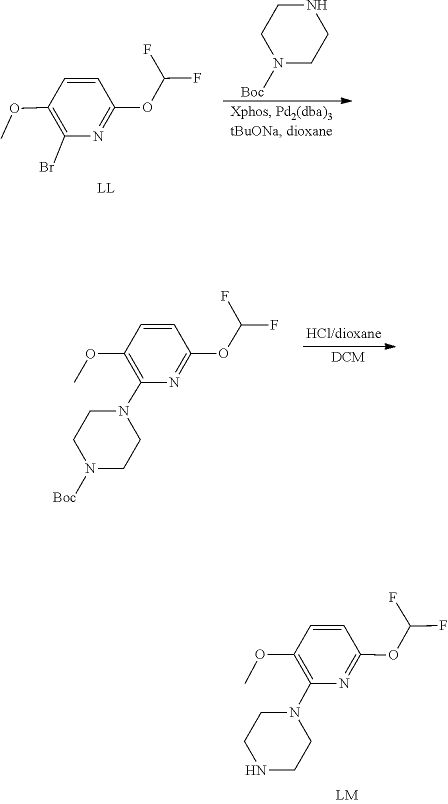 [SMILES: COc1ccc(OC(F)F)nc1Br ; CC(C)(C)OC(=O)N1CCNCC1 ; COc1ccc(OC(F)F)nc1N1CCN(C(=O)OC(C)(C)C)CC1 ; COc1ccc(OC(F)F)nc1N1CCNCC1]

### Step 1—Tert-butyl 4-(6-(difluoromethoxy)-3-methoxypyridin-2-yl)piperazine-1-carboxylate

A mixture of 2-bromo-6-(difluoromethoxy)-3-methoxypyridine (0.4 g, 1.6 mmol, Intermediate LL), tert-butyl piperazine-1-carboxylate (293 mg, 1.57 mmol, CAS #5726-71-6), XPhos (75.0 mg, 157 μmol), Pd₂(dba)₃ (72.1 mg, 78.7 μmol) and tBuONa (378 mg, 3.94 mmol) in dioxane (2 mL) was degassed and purged with N₂ three times. Then the mixture was stirred at 100° C. for 4 hrs under N₂ atmosphere. On completion, the reaction mixture was concentrated under reduced pressure and purified by column chromatography (SiO₂, Petroleum ether/Ethyl acetate=1/10 to 1/2) to give the title compound (400 mg, 54% yield) as a white solid. LC-MS (ESI⁺) m/z 382.1 (M+23)⁺.

### Step 2—1-(6-(Difluoromethoxy)-3-methoxypyridin-2-yl)piperazine

To a solution of tert-butyl 4-(6-(difluoromethoxy)-3-methoxypyridin-2-yl)piperazine-1-carboxylate (0.2 g, 556 μmol) in DCM (2 mL) was added HCl/dioxane (4 M, 139 μL). The mixture was then stirred at 25° C. for 2 hrs. On completion, the reaction mixture was filtered and concentrated under reduced pressure to give the title compound (0.14 g, HCl) as a white solid. LC-MS (ESI⁺) m/z 260.0 (M+H)⁺.

### 2-Ethyl-1H-imidazole (CAS #1072-62-4) (Intermediate LN)

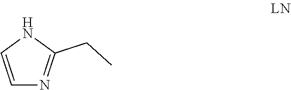

### [7-fluoro-2-[4-(3-methoxy-2-pyridyl)piperazine-1-carbonyl]-6-[1-[3-(triazol-1-yl) propanoyl]-3,6-dihydro-2H-pyridin-5-yl]-1H-indol-4-yl]boronic acid (Intermediate LO)

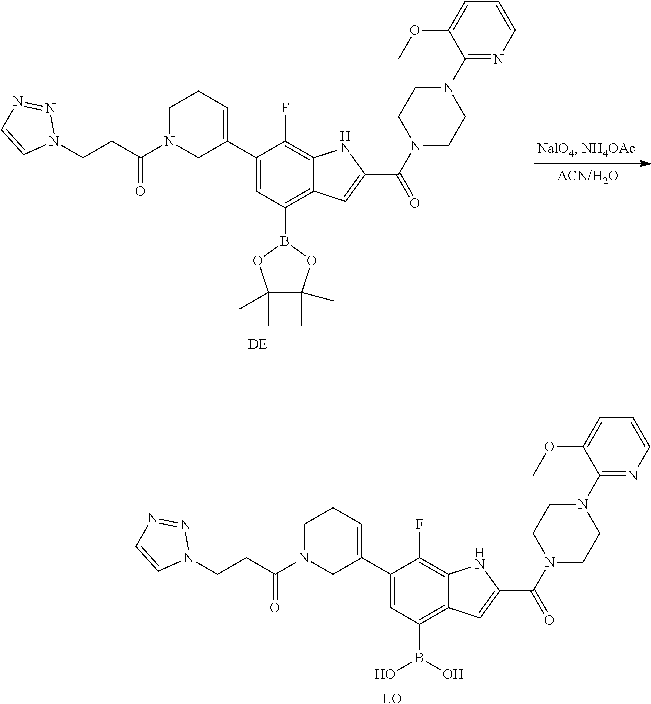

To a solution of 1-[5-[7-fluoro-2-[4-(3-methoxy-2-pyridyl)piperazine-1-carbonyl]-4-(4,4,5,5-tetramethyl-1,3,2-dioxaborolan-2-yl)-1H-indol-6-yl]-3,6-dihydro-2H-pyridin-1-yl]-3-(triazol-1-yl)propan-1-one (130 mg, 190 μmol, Intermediate DE) in ACN (5 mL) and H₂O (2.5 mL) was added NaIO₄ (121 mg, 569 μmol, 31.5 μL) and NH₄OAc (29.2 mg, 379 μmol) at 0° C. The mixture was then stirred at 20° C. for 12 hrs. On completion, the mixture was concentrated under vacuum to give a residue. The crude residue was purified by reversed-phase HPLC (FA condition) to give the title compound (60 mg, 53% yield) as a yellow solid. ¹H NMR (400 MHz, DMSO-d₆) δ=11.96 (s, 1H), 8.12 (d, J=8.8 Hz, 1H), 8.09-8.04 (m, 2H), 7.81 (dd, J=1.2, 4.8 Hz, 1H), 7.73-7.64 (m, 1H), 7.54-7.47 (m, 1H), 7.28 (dd, J=1.2, 8.0 Hz, 1H), 7.12 (br s, 1H), 6.93 (dd, J=4.8, 8.0 Hz, 1H), 6.16-6.06 (m, 1H), 4.70-4.58 (m, 2H), 4.40-4.26 (m, 2H), 3.88-3.82 (m, 4H), 3.82 (s, 3H), 3.68-3.57 (m, 2H), 3.39-3.33 (m, 4H), 3.13-3.05 (m, 2H), 2.36-2.25 (m, 2H).

### 1-(6-Chloro-3-methoxypyridin-2-yl)piperazine (Intermediate LP)

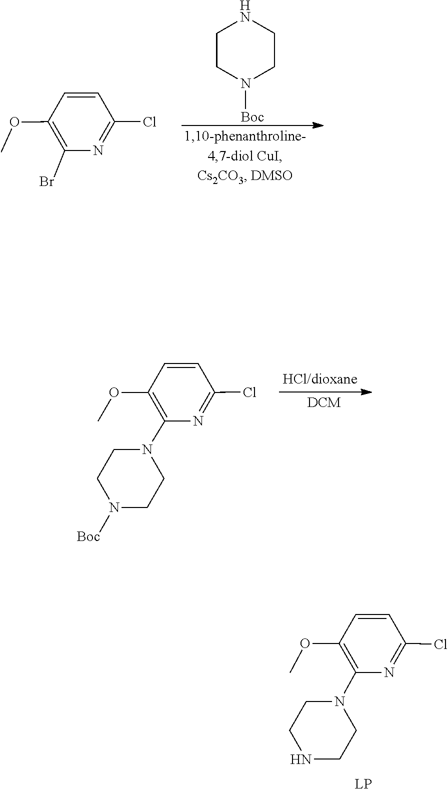

### Step 1—Tert-butyl 4-(6-chloro-3-methoxypyridin-2-yl)piperazine-1-carboxylate

A mixture of 2-bromo-6-chloro-3-methoxypyridine (0.5 g, 2.3 mmol, CAS #1256819-37-0), tert-butyl piperazine-1-carboxylate (600 mg, 2.70 mmol, HCl, CAS #57260-71-6), Cs₂CO₃ (2.93 g, 8.99 mmol), 1,10-phenanthroline-4,7-diol (476 mg, 2.25 mmol) and CuI (214 mg, 1.12 mmol) in DMSO (5 mL) was degassed and purged with N₂ three times. Then the mixture was stirred at 100° C. for 12 hrs under N₂ atmosphere. On completion, the reaction mixture was concentrated under reduced pressure and purified by prep-HPLC (FA condition) to give the title compound (0.3 g, 18% yield, FA) as a white solid. LC-MS (ESI⁺) m/z 228.0 (M−100)⁺.

### Step 2—1-(6-Chloro-3-methoxypyridin-2-yl)piperazine

To a solution of tert-butyl 4-(6-chloro-3-methoxypyridin-2-yl)piperazine-1-carboxylate (0.3 g, 920 μmol) in DCM (3 mL) was added HCl/dioxane (4 M, 228 μL). The mixture was then stirred at 25° C. for 2 hrs. On completion, the reaction mixture was filtered and concentrated under reduced pressure to give the title compound (0.2 g, HCl) as a white solid. LC-MS (ESI⁺) m/z 228.1 (M+H)⁺.

### (4-Bromo-6-chloro-7-fluoro-1H-indol-2-yl)(4-(5-fluoro-3-methoxypyridin-2-yl)piperazin-1-yl)methanone (Intermediate LO)

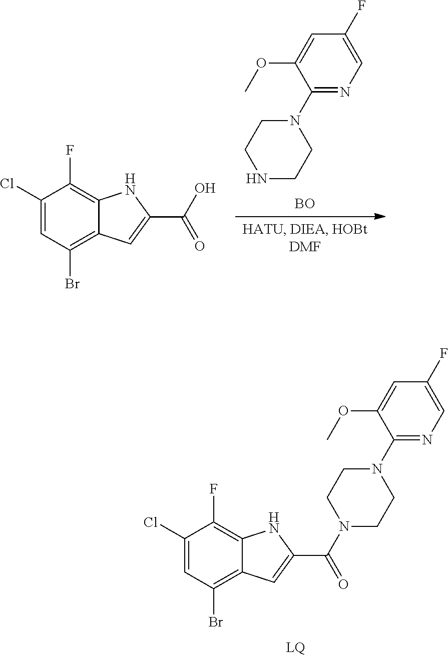

To a solution of 4-bromo-6-chloro-7-fluoro-1H-indole-2-carboxylic acid (3 g, 10 mmol, synthesized via Steps 1-6 of Intermediate C) in DMF (30 mL) was added HATU (4.68 g, 12.3 mmol), DIEA (7.95 g, 61.6 mmol), HOBt (1.66 g, 12.3 mmol) and 1-(5-fluoro-3-methoxy-2-pyridyl)piperazine (2.60 g, 12.3 mmol, Intermediate BO). The mixture was then stirred at 0-25° C. for 30 mins. On completion, the reaction mixture water was added then the mixture was filtered and the filter cake was dried in vacuo to give the title compound (4 g, 79% yield) as a yellow solid. LC-MS (ESI⁺) m/z 486.9 (M+H)⁺.

### (6-chloro-7-fluoro-4-propyl-1H-indol-2-yl)(4-(5-fluoro-3-methoxypyridin-2-yl)piperazin-1-yl)methanone (Intermediate LR), (6-chloro-4-cyclopropyl-7-fluoro-1H-indol-2-yl)(4-(5-fluoro-3-methoxypyridin-2-yl)piperazin-1-yl)methanone (Intermediate LS) and (6-chloro-7-fluoro-4-hexyl-1H-indol-2-yl)(4-(5-fluoro-3-methoxypyridin-2-yl)piperazin-1-yl)methanone (Intermediate LT)

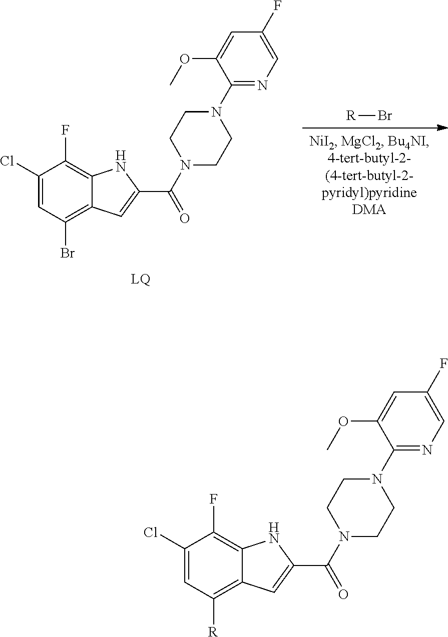

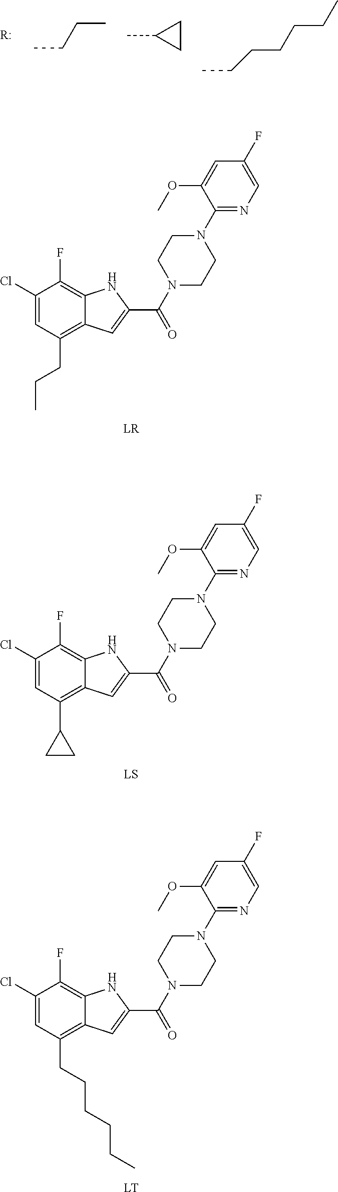

To a solution of (4-bromo-6-chloro-7-fluoro-1H-indol-2-yl)-[4-(5-fluoro-3-methoxy-2-pyridyl)piperazin-1-yl]methanone (300 mg, 618 μmol, Intermediate LQ) and the corresponding bromide (865 μmol, 1.4 eq) in DMA (8 mL) was added diiodonickel (19.3 mg, 61.8 μmol), dichloromagnesium (58.8 mg, 618 μmol, 1 eq), tetrabutylammonium;iodide (228 mg, 618 μmol) and 4-tert-butyl-2-(4-tert-butyl-2-pyridyl)pyridine (16.6 mg, 61.8 μmol). The mixture was then stirred at 25° C. for 12 hrs under N₂ atmosphere. On completion, the reaction mixture was concentrated under reduced pressure to remove solvent. Then the mixture was purified by reversed-phase HPLC (0.1% FA condition) to give the title compounds.

### 1-(2,4-Difluoro-3-methoxyphenyl)piperazine (Intermediate LU)

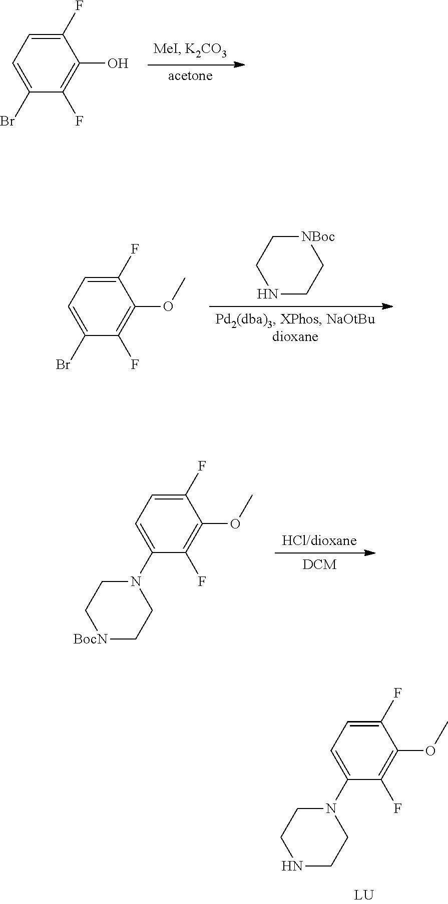

### Step 1—1-Bromo-2,4-difluoro-3-methoxybenzene

To a solution of 3-bromo-2, 6-difluorophenol (500 mg, 2.39 mmol, CAS #221220-99-1) in acetone (5 mL) was added K₂CO₃ (338 mg, 2.44 mmol) and the mixture was stirred at 20° C. for 30 min. Finally, MeI (684 mg, 4.82 mmol, 300 μL) was added to the mixture and the mixture was stirred at 20° C. for 3 hrs. On completion, the mixture was concentrated under reduced pressure to give the title compound (417 mg, 78% yield) as a yellow solid. ¹H NMR (400 MHz, DMSO-d₆) δ=3.95 (s, 3H) 7.18 (m, 1H) 7.44 (m, 1H).

### Step 2—Tert-butyl 4-(2,4-difluoro-3-methoxyphenyl)piperazine-1-carboxylate

To a solution of 1-bromo-2, 4-difluoro-3-methoxybenzene (350 mg, 1.57 mmol) and tert-butyl piperazine-1-carboxylate (350.76 mg, 1.88 mmol, CAS #57260-71-6) in dioxane (5 mL) was added Cs₂CO₃ (1.53 g, 4.71 mmol). Finally, 1,3-bis[2,6-bis(1-ethylpropyl)phenyl]-4,5-dichloro-2H-imidazol-1-ium-2-ide 3-chloropyridine dichloropalladium (135.06 mg, 156.94 μmol) was added to the mixture and the mixture was stirred at 100° C. for 12 hrs. On completion, the mixture was diluted with water (10 mL) and extracted with ethyl acetate (3×30 mL) and the organic phase was washed with brine (3×30 mL), dried over sodium sulfate, filtered and concentrated under reduced pressure to give the residue. The residue was purified by flash silica gel chromatography (ISCO®; 4 g SepaFlash® Silica Flash Column, Eluent of 0˜10% Ethyl acetate/Petroleum ether gradient @40 mL/min) to give the title compound (300 mg, 58% yield) as a yellow oil. ¹H NMR (400 MHz, DMSO-d₆) δ=7.10-7.00 (m, 1H), 6.77 (dt, J=5.2, 9.2 Hz, 1H), 3.89 (s, 3H), 3.45 (br d, J=4.4 Hz, 4H), 2.92-2.86 (m, 4H), 1.41 (s, 9H).

### Step 3—1-(2,4-Difluoro-3-methoxyphenyl)piperazine

To a solution of tert-butyl 4-(2,4-difluoro-3-methoxyphenyl)piperazine-1-carboxylate (100 mg, 304.55 μmol) in DCM (2 mL) was added HCl/dioxane (4 M, 2 mL) and the mixture was stirred at 20° C. for 1 hr. On completion, the mixture was concentrated under reduced pressure to give the title compound (78 mg, HCl) as a white solid. ¹H NMR (400 MHz, DMSO-d₆) δ=9.23 (s, 2H), 7.11-7.03 (m, 1H), 6.83 (dt, J=5.2, 9.2 Hz, 1H), 3.90 (s, 3H), 3.24-3.15 (m, 8H).

### 3-Piperazin-1-ylpyridine-2-carbonitrile (Intermediate LV)

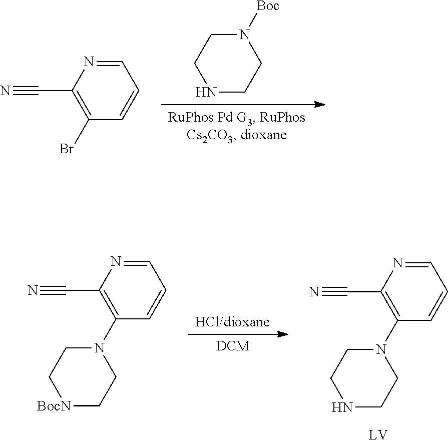

### Step 1—tert-butyl 4-(2-cyano-3-pyridyl)piperazine-1-carboxylate

A mixture of 3-bromopyridine-2-carbonitrile (500 mg, 2.73 mmol, CAS #55758-02-6), tert-butyl piperazine-1-carboxylate (610.64 mg, 3.28 mmol, CAS #57260-71-6), RuPhos Pd G₃ (114.25 mg, 136.61 μmol), RuPhos (127.49 mg, 273.22 μmol) and Cs₂CO₃ (1.78 g, 5.46 mmol) in dioxane (10 mL) was degassed and purged with N₂ three times. Then the mixture was stirred at 110° C. for 12 hrs under N₂ atmosphere. On completion, the reaction mixture was diluted with H₂O (20 mL) and extracted with EtOAc (20 mL×2). The combined organic layers were washed with brine (20 mL), dried over Na₂SO₄, filtered and concentrated under reduced pressure to give a residue. The residue was purified by flash silica gel chromatography (ISCO®; 12 g SepaFlash® Silica Flash Column, Eluent of 0˜20% EtOAc/PE gradient @100 mL/min) to give the title compound (500 mg, 60% yield) as a yellow gum. LC-MS (ESI⁺) m/z 289.1 (M+H)⁺; ¹H NMR (400 MHz, CDCl₃-d) δ=8.31 (dd, J=1.2, 4.4 Hz, 1H), 7.46-7.40 (m, 1H), 7.39-7.33 (m, 1H), 3.72-3.62 (m, 4H), 3.24-3.15 (m, 4H), 1.49 (s, 9H).

### Step 2—3-Piperazin-1-ylpyridine-2-carbonitrile

To a solution of tert-butyl 4-(2-cyano-3-pyridyl)piperazine-1-carboxylate (200 mg, 700 μmol) in DCM (1 mL) was added HCl/dioxane (2 M, 2.00 mL). The mixture was stirred at 20° C. for 1 hr. On completion, the reaction mixture was diluted with water (10 mL) and adjusted the pH to 8 with NaHCO₃, and extracted with DCM (10 mL×3). The combined organic layers were dried over Na₂SO₄, filtered and concentrated under reduced pressure to give the title compound (113 mg, 78% yield) as a yellow solid. LC-MS (ESI⁺) m/z 188.9 (M+H)⁺.

### 1-(3-Cyclopropylpyridin-2-yl)piperazine (Intermediate LW)

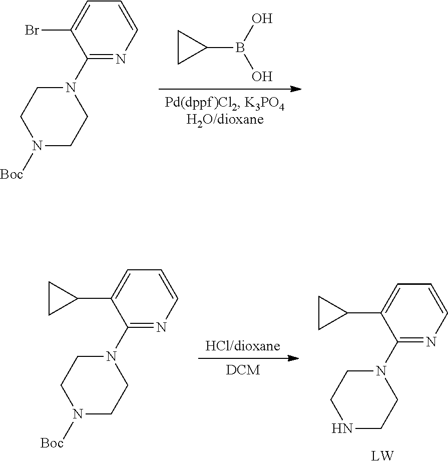

### Step 1—Tert-butyl 4-(3-cyclopropylpyridin-2-yl)piperazine-1-carboxylate

A mixture of tert-butyl 4-(3-bromo-2-pyridyl)piperazine-1-carboxylate (120 mg, 350 μmol, synthesized via Step 1 of Intermediate R), cyclopropylboronic acid (35 mg, 407 μmol), K₃PO₄ (230 mg, 1.08 mmol), and Pd(dppf)Cl₂·CH₂Cl₂ (14.3 mg, 17.5 μmol) in dioxane (1 mL) and H₂O (0.1 mL) was degassed and purged with N₂ three times. Then the mixture was stirred at 100° C. for 2 hr under N₂ atmosphere. On completion, the solution was filtered and the solvent removed in vacuo. The crude was purified by column chromatography (SiO₂, PE/EA=10/1 to 3/1) to give the title compound (77 mg, 69% yield) as a yellow oil. LC-MS (ESI⁺) m/z 304.1 (M+H)⁺.

### Step 2—1-(3-Cyclopropylpyridin-2-yl)piperazine

To a solution of tert-butyl 4-(3-cyclopropyl-2-pyridyl)piperazine-1-carboxylate (77 mg, 250 μmol) in DCM (2 mL) was added HCl/dioxane (2 M, 2 mL). Then the mixture was stirred at 20° C. for 1 hr. On completion, the mixture was concentrated to give the title compound (60 mg, 99% yield, HCl) as a white solid. LC-MS (ESI⁺) m/z 204.1 (M+H)⁺.

### (6-Chloro-7-fluoro-4-(4-methoxypyridin-3-yl)-1H-indol-2-yl)(4-(5-fluoro-3-methoxypyridin-2-yl)piperazin-1-yl)methanone (Intermediate LX)

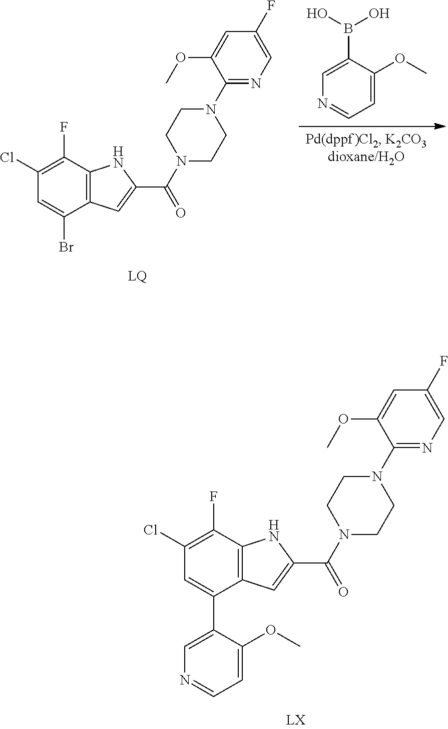

To a solution of (4-bromo-6-chloro-7-fluoro-1H-indol-2-yl)(4-(5-fluoro-3-methoxypyridin-2-yl)piperazin-1-yl)methanone (200 mg, 411 μmol, Intermediate LQ), (4-methoxypyridin-3-yl)boronic acid (94.4 mg, 617 μmol, CAS #355004-67-0) in dioxane (2 mL), and H₂O (0.5 mL) was added K₂CO₃ (170 mg, 1.24 mmol) and Pd(dppf)Cl₂ (30.1 mg, 41.1 μmol). The reaction was then stirred at 80° C. for 1 hr under nitrogen atmosphere. On completion, the reaction mixture was concentrated in vacuo to give the crude residue. The crude residue was purified by column chromatography (SiO₂, Petroleum ether:Ethyl acetate=10/1 to 0/1) to give the title compound (130 mg, 60% yield) as a yellow solid. LC-MS (ESI⁺) m/z 514.1 (M+H)⁺.

### (6-Chloro-7-fluoro-4-(3-methoxypyridin-4-yl)-1H-indol-2-yl)(4-(5-fluoro-3-methoxypyridin-2-yl)piperazin-1-yl)methanone (Intermediate LY)

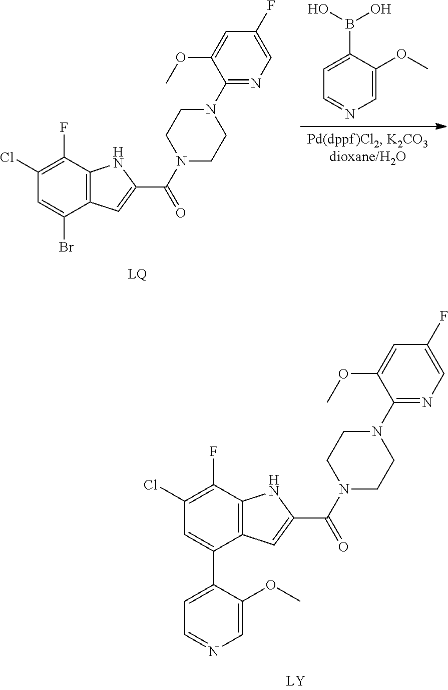

To a solution of (4-bromo-6-chloro-7-fluoro-1H-indol-2-yl)(4-(5-fluoro-3-methoxypyridin-2-yl)piperazin-1-yl)methanone (200 mg, 411 μmol, Intermediate LQ), (3-methoxypyridin-4-yl)boronic acid (94.4 mg, 617 μmol, CAS #1008506-24-8) in dioxane (2 mL) and H₂O (0.5 mL) was added K₂CO₃ (170 mg, 1.24 mmol), and Pd(dppf)Cl₂ (30.1 mg, 41.1 μmol). Then the reaction was stirred at 80° C. for 1 hr under nitrogen atmosphere. On completion, the reaction mixture was concentrated in vacuo to give the crude residue. The crude residue was purified by column chromatography (SiO₂, Petroleum ether: Ethyl acetate=10/1 to 0/1) to give the title compound (150 mg, 71% yield) as a yellow solid. LC-MS (ESI⁺) m/z 514.1 (M+H)⁺.

### 1-Methyl-5,6-dihydro-4H-pyrrolo[3,4-c]pyrazole (CAS #762233-62-5) (Intermediate LZ)

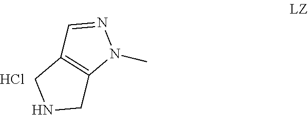

### 2-Methoxy-3-(piperazin-1-yl)pyrazine (Intermediate MA)

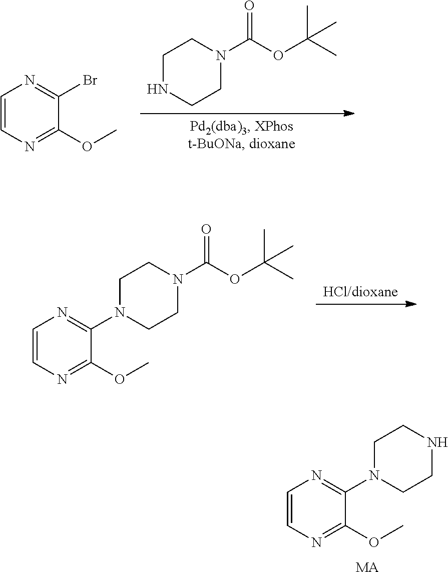

### Step 1—tert-butyl 4-(3-methoxypyrazin-2-yl)piperazine-1-carboxylate

A mixture of 2-bromo-3-methoxypyrazine (200 mg, 1.06 mmol) and tert-butyl piperazine-1-carboxylate (197 mg, 1.06 mmol) in dioxane (2 mL) and was added Pd₂(dba)₃ (96.9 mg, 106 μmol), t-BuONa (305.07 mg, 3.17 mmol) and XPhos (50.4 mg, 106 μmol). The mixture was then stirred at 100° C. for 6 hrs under N₂ atmosphere. The reaction mixture was filtered and concentrated under reduced pressure to give a residue. The crude product was purified by MPLC(SiO2, PE:EA=10:1 to 0:1) to give the title compound (500 mg, 69% yield) as a yellow oil. LC-MS (ESI+) m/z 295.1 (M+H)⁺.

### Step 2—2-Methoxy-3-(piperazin-1-yl)pyrazine

To a solution of tert-butyl 4-(3-methoxypyrazin-2-yl)piperazine-1-carboxylate (500 mg, 1.70 mmol) was added HCl/dioxane (2 M, 5 mL). The mixture was then stirred at 25° C. for 2 hrs. On completion, the reaction mixture was filtered and concentrated to give the title compound (100 mg, HCl) as a white solid. LC-MS (ESI+) m/z 195.0 (M+H)⁺.

### Tert-butyl 9-bromo-3-azaspiro[5.5]undecane-3-carboxylate (Intermediate MB)

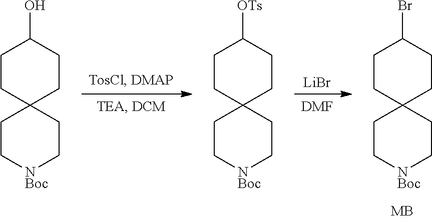

### Step 1—Tert-butyl 9-(tosyloxy)-3-azaspiro[5.5]undecane-3-carboxylate

To a solution of tert-butyl 9-hydroxy-3-azaspiro[5.5]undecane-3-carboxylate (2.5 g, 9.3 mmol, CAS #918644-73-2) in DCM (20 mL) was added TEA (2.82 g, 27.8 mmol, 3.88 mL), DMAP (113 mg, 928 μmol) and TosCl (3.54 g, 18.5 mmol) at 0° C. The reaction was then stirred at 25° C. for 12 hrs. On completion, the reaction mixture was concentrated in vacuo to give the crude residue. The residue was purified by column chromatography (SiO₂, Petroleum ether:Ethyl acetate=10/1 to 5/1) to give the title compound (2 g, 50% yield) as a white solid. ¹H NMR (400 MHz, DMSO-d₆) δ=7.79 (d, J=8.4 Hz, 2H), 7.47 (d, J=8.4 Hz, 2H), 4.49 (tt, J=4.0, 7.6 Hz, 1H), 3.26-3.21 (m, 4H), 2.42 (s, 3H), 1.62-1.46 (m, 6H), 1.37 (s, 9H), 1.33-1.29 (m, 2H), 1.25-1.20 (m, 4H).

### Step 2—Tert-butyl 9-bromo-3-azaspiro[5.5]undecane-3-carboxylate

To a solution of tert-butyl 9-(tosyloxy)-3-azaspiro[5.5]undecane-3-carboxylate (2 g, 5 mmol) in DMF (20 mL) was added LiBr (1.23 g, 14.1 mmol, 355 μL). The reaction was stirred at 60° C. for 12 hrs. On completion, the reaction mixture was concentrated in vacuo to give the title compound (1.2 g) as a colorless oil. ¹H NMR (400 MHz, DMSO-d₆) δ=3.43-3.37 (m, 1H), 3.31-3.23 (m, 3H), 2.06-1.95 (m, 2H), 1.90-1.79 (m, 2H), 1.66-1.55 (m, 1H), 1.46-1.43 (m, 1H), 1.40 (d, J=1.6 Hz, 9H), 1.32-1.28 (m, 3H).

### Tert-butyl 9-(6-chloro-7-fluoro-2-(4-(3-methoxypyridin-2-yl)piperazine-1-carbonyl)-1H-indol-4-yl)-3-azaspiro[5.5]undecane-3-carboxylate (Intermediate MC)

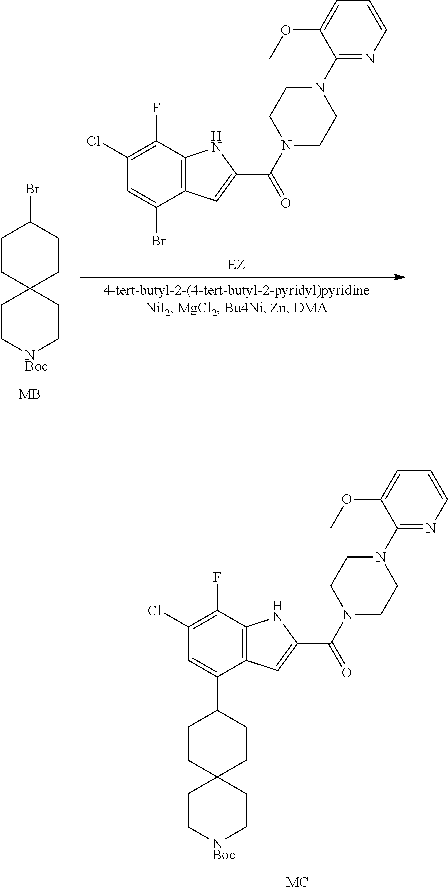 [SMILES: CC(C)(C)OC(=O)N1CCC2(CCC(Br)CC2)CC1 ; COc1cccnc1N1CCN(C(=O)c2cc3c(Br)cc(Cl)c(F)c3[nH]2)CC1 ; "4-tert-butyl-2-(4-tert-butyl-2-pyridyl)pyridineNiI2, MgCl2, Bu4Ni, Zn, DMA" ; "EZ" ; COc1cccnc1N1CCN(C(=O)c2cc3c(C4CCC5(CC4)CCN(C(=O)OC(C)(C)C)CC5)cc(Cl)c(F)c3[nH]2)CC1]

To a solution of tert-butyl 9-bromo-3-azaspiro[5.5]undecane-3-carboxylate (497 mg, 1.50 mmol, Intermediate MB) in DMA (10 mL) was added (4-bromo-6-chloro-7-fluoro-1H-indol-2-yl)(4-(3-methoxypyridin-2-yl)piperazin-1-yl)methanone (500 mg, 1.07 mmol, Intermediate EZ), diiodonickel (33.4 mg, 106 μmol, 5.73 μL), MgCl₂ (101 mg, 1.07 mmol, 43.8 μL), Bu₄NI (394 mg, 1.07 mmol) and 4-tert-butyl-2-(4-tert-butyl-2-pyridyl)pyridine (28.6 mg, 106 μmol). The reaction was then stirred at 30° C. for 12 hrs under N₂ atmosphere. On completion, the reaction mixture was concentrated in vacuo to give the crude residue. The crude residue was purified by reversed-phase HPLC (0.1% FA condition) to give the title compound (450 mg, 51% yield) as a brown solid. LC-MS (ESI⁺) m/z 640.1 (M+H)⁺.

### 1-(5-(4,4,5,5-Tetramethyl-1,3,2-dioxaborolan-2-yl)-3,6-dihydropyridin-1(2H)-yl)-3-(thiazol-2-yl)propan-1-one (Intermediate MD)

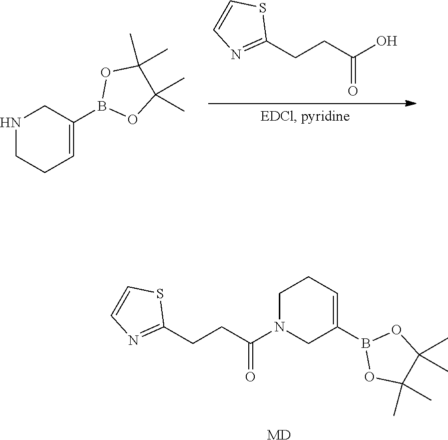

To a solution of 3-(thiazol-2-yl)propanoic acid (1 g, 6 mmol, CAS #144163-65-5) in Py (20 mL) was added EDCI (2.07 g, 10.81 mmol) at 20° C., and the mixture was stirred at 20° C. for 30 min. Next, 5-(4,4,5,5-tetramethyl-1,3,2-dioxaborolan-2-yl)-1,2,3,6-tetrahydropyridine (2.00 g, 9.54 mmol, synthesized via Step 1 of Intermediate U) was added to the mixture at 0° C. and the mixture was stirred at 20° C. for 12 hrs. On completion, the mixture was diluted with water (10 mL) and extracted with ethyl acetate (3×50 mL). The organic phase was washed with brine (3×50 mL), dried over sodium sulfate, filtered and concentrated under reduced pressure to give the residue. The residue was purified by flash silica gel chromatography (ISCO®; 20 g SepaFlash® Silica Flash Column, Eluent of 0˜35% Ethyl acetate/Petroleum ether gradient @100 mL/min) to give the title compound (2 g, 45% yield) as a yellow solid. ¹H NMR (400 MHz, DMSO-d₆) δ=1.21 (d, J=2.4 Hz, 12H) 2.07-2.25 (m, 2H) 2.83 (m, 2H) 3.21 (br t, J=7.2 Hz, 2H) 3.45-3.54 (m, 2H) 3.96 (br s, 2H) 6.49-6.60 (m, 1H) 7.54 (d, J=3.2 Hz, 1H) 7.66 (d, J=3.2 Hz, 1H).

### Methyl 4-chloro-7-fluoro-6-(1-(3-(thiazol-2-yl)propanoyl)-1,2,5,6-tetrahydropyridin-3-yl)-1H-indole-2-carboxylate (Intermediate ME)

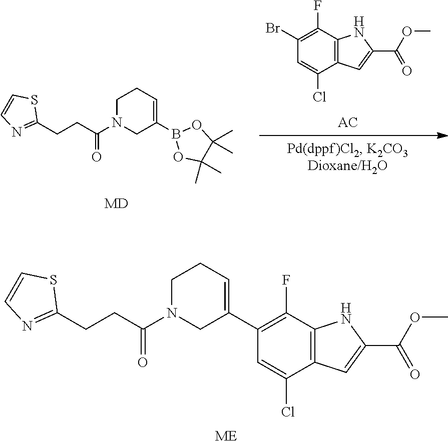

To a solution of 1-(5-(4,4,5,5-tetramethyl-1,3,2-dioxaborolan-2-yl)-3,6-dihydropyridin-1(2H)-yl)-3-(thiazol-2-yl)propan-1-one (1 g, 2.87 mmol, Intermediate MD) and methyl 6-bromo-4-chloro-7-fluoro-1H-indole-2-carboxylate (1.06 g, 3.45 mmol, Intermediate AC) in dioxane (10 mL) and H₂O (1 mL) was added K₂CO₃ (1.19 g, 8.61 mmol). Finally, Pd(dppf)Cl₂ (210.10 mg, 287.14 μmol) was added to the mixture and the mixture was stirred at 80° C. for 4 hrs. Upon completion, the mixture was diluted with water (10 mL) and extracted with ethyl acetate (3×30 mL). The organic phase was washed with brine (3×30 mL), dried over sodium sulfate, filtered and concentrated under reduced pressure to give the residue. The residue was purified by flash silica gel chromatography (ISCO®; 20 g SepaFlash® Silica Flash Column, Eluent of 0-65% Ethyl acetate/Petroleum ether gradient @100 mL/min) to give the title compound (1.17 g, 91% yield) as a yellow solid. ¹H NMR (400 MHz, DMSO-d₆) δ=2.21-2.43 (m, 2H) 2.84-3.01 (m, 2H) 3.24 (m, 2H) 3.57-3.69 (m, 2H) 3.90 (s, 3H) 4.32 (br s, 2H) 6.15 (br s, 1H) 7.07-7.23 (m, 2H) 7.50-7.59 (m, 1H) 7.61-7.74 (m, 1H) 12.90 (br s, 1H).

### 7-Fluoro-4-(4-methoxypyridin-3-yl)-6-(1-(3-(thiazol-2-yl)propanoyl)-1,2,5,6-tetrahydropyridin-3-yl)-1H-indole-2-carboxylic acid (Intermediate MF)

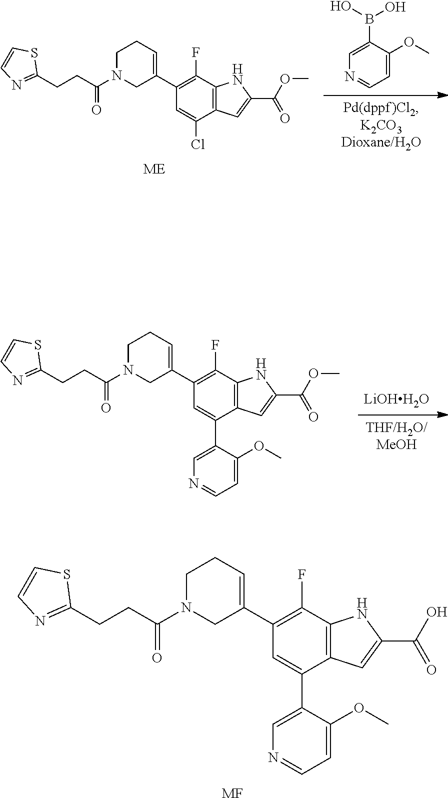

### Step 1—Methyl 7-fluoro-4-(4-methoxypyridin-3-yl)-6-(1-(3-(thiazol-2-yl)propanoyl)-1,2,5,6-tetrahydropyridin-3-yl)-1H-indole-2-carboxylate

To a solution of methyl 4-chloro-7-fluoro-6-(1-(3-(thiazol-2-yl) propanoyl)-1, 2, 5, 6-tetrahydropyridin-3-yl)-1H-indole-2-carboxylate (300 mg, 669 μmol, Intermediate ME) and (4-methoxypyridin-3-yl)boronic acid (153.66 mg, 1.00 mmol, CAS #355004-67-0) in dioxane (3 mL) and H₂O (0.3 mL) was added K₃PO₄ (426.51 mg, 2.01 mmol). Finally, XPhos Pd G3 (56.69 mg, 66.98 μmol) was added to the mixture at N₂ and the mixture was stirred at 90° C. for 12 hrs. Upon completion, the mixture was diluted with water (10 mL) and extracted with ethyl acetate (3×30 mL). The organic phase was washed with brine (3×30 mL), dried over sodium sulfate and filtered and concentrated under reduced pressure to give the residue. The residue was purified by flash silica gel chromatography (ISCO®; 12 g SepaFlash® Silica Flash Column, Eluent of 0˜10% Dichloromethane:Methanol gradient @40 mL/min) to the title compound (120 mg, 34% yield) as a yellow oil. ¹H NMR (400 MHz, DMSO-d₆) δ=2.24-2.40 (m, 2H) 2.92 (m, 2H) 3.24 (m, 2H) 3.65 (m, 2H) 3.81 (s, 3H) 3.85 (s, 3H) 4.38 (br s, 2H) 6.17 (br s, 1H) 6.82 (d, J=2.4 Hz, 1H) 6.95-7.09 (m, 1H) 7.21 (d, J=5.6 Hz, 1H) 7.50-7.57 (m, 1H) 7.61-7.69 (m, 1H) 8.38 (s, 1H) 8.52 (d, J=5.6 Hz, 1H) 12.58 (br s, 1H).

### Step 2—7-Fluoro-4-(4-methoxypyridin-3-yl)-6-(1-(3-(thiazol-2-yl)propanoyl)-1,2,5,6-tetrahydropyridin-3-yl)-1H-indole-2-carboxylic acid

To a solution of methyl 7-fluoro-4-(4-methoxypyridin-3-yl)-6-(1-(3-(thiazol-2-yl) propanoyl)-1, 2, 5, 6-tetrahydropyridin-3-yl)-1H-indole-2-carboxylate (210 mg, 403.40 μmol) in THF (0.7 mL), H₂O (0.7 mL) and MeOH (0.7 mL) was added LiOH·H₂O (67.71 mg, 1.61 mmol). The mixture was then stirred at 20° C. for 12 hrs. Upon completion, the mixture was adjusted to pH of 5 with 1 M HCl solution, then extracted with ethyl acetate (3×20 mL). The organic phase was concentrated under reduced pressure to give the title compound. (180 mg, 88% yield) as a yellow solid. ¹H NMR (400 MHz, DMSO-d₆) δ=2.22-2.40 (m, 2H) 2.87-2.99 (m, 2H) 3.26 (m, 2H) 3.64 (m, 2H) 4.06 (s, 3H) 4.37 (br s, 2H) 6.19 (br s, 1H) 6.96 (br d, J=2.0 Hz, 1H) 7.11-7.24 (m, 1H) 7.55-7.64 (m, 1H) 7.66-7.75 (m, 1H) 7.78 (d, J=7.2 Hz, 1H) 8.74-8.81 (m, 1H) 8.90 (br d, J=6.8 Hz, 1H) 12.54 (s, 1H).

### 3-Chloro-4-ethylpyridine (CAS #38749-76-7) (Intermediate MG)

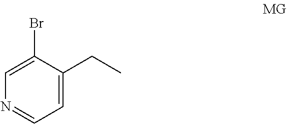

### 1-(5-Fluoro-2-methoxypyridin-3-yl)piperazine (Intermediate MH)

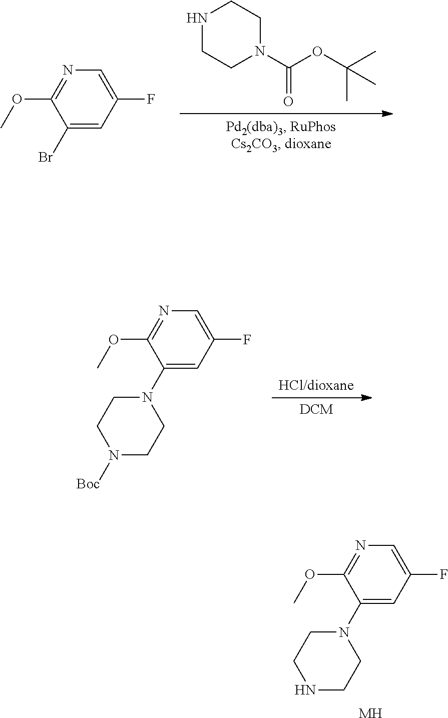

### Step 1—Tert-butyl 4-(5-fluoro-2-methoxypyridin-3-yl)piperazine-1-carboxylate

To a solution of tert-butyl piperazine-1-carboxylate (994 mg, 5.34 mmol, CAS #57260-71-6) and 3-bromo-5-fluoro-2-methoxypyridine (1 g, 5 mmol, CAS #884494-81-9) in dioxane (20 mL) was added Cs₂CO₃ (4.74 g, 14.56 mmol). Finally, RuPhos (453 mg, 970 μmol) and Pd₂(dba)₃ (444 mg, 485 μmol) were added to the mixture at N₂ and the mixture was stirred at 100° C. for 12 hrs. On completion, the mixture was diluted with water (10 mL) and extracted with ethyl acetate (3×30 mL). The organic phase was washed with brine (3×30 mL), dried over sodium sulfate, filtered and concentrated under reduced pressure to give the residue. The residue was purified by flash silica gel chromatography (ISCO®; 20 g SepaFlash® Silica Flash Column, Eluent of 0-10% Ethyl acetate/Petroleum ether gradient @100 mL/min) to give the title compound (1.20 g, 79% yield) as a yellow solid. ¹H NMR (400 MHz, DMSO-d6) δ=1.42 (br s, 9H) 2.98 (br s, 4H) 3.45 (br s, 4H) 3.87 (br s, 3H) 7.05-7.35 (m, 1H) 7.64-7.84 (m, 1H).

### Step 2—1-(5-fluoro-2-methoxypyridin-3-yl)piperazine

To a solution of tert-butyl 4-(5-fluoro-2-methoxypyridin-3-yl) piperazine-1-carboxylate (1.2 g, 3.9 mmol) in DCM (13 mL) was added HCl/dioxane (4 M, 963 μL) and the mixture was stirred at 20° C. for 2 hrs. On completion, the mixture was concentrated under reduced pressure to give the title compound 1 (900 mg, HCl) as a yellow solid. ¹H NMR (400 MHz, DMSO-d₆) δ=3.19 (br s, 4H) 3.24-3.31 (m, 4H) 3.87 (s, 3H) 7.28 (m, 1H) 7.75 (d, J=2.4 Hz, 1H) 8.58-8.84 (m, 1H) 9.35-9.61 (m, 2H).

### 1-(5-(4-Chloro-7-fluoro-2-(4-(5-fluoro-2-methoxypyridin-3-yl)piperazine-1-carbonyl)-1H-indol-6-yl)-3,6-dihydropyridin-1(2H)-yl)ethan-1-one (Intermediate MI)

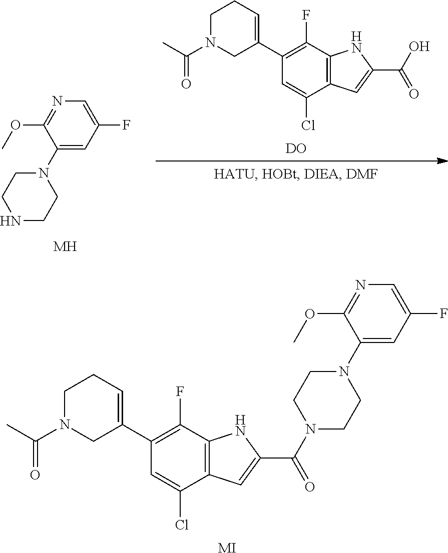

To a solution of 6-(1-acetyl-1,2,5,6-tetrahydropyridin-3-yl)-4-chloro-7-fluoro-1H-indole-2-carboxylic acid (420 mg, 1.25 mmol, Intermediate DO) in DMF (5 mL) was added HATU (711.35 mg, 1.87 mmol), HOBt (252.80 mg, 1.87 mmol) and DIEA (483.59 mg, 3.74 mmol, 651.73 μL) at 0° C. and the mixture was stirred for 30 min. Finally, 1-(5-fluoro-2-methoxypyridin-3-yl)piperazine (395.19 mg, 1.87 mmol, Intermediate MH) was added to the mixture and the mixture was stirred at 20° C. for 2 hrs. On completion, the mixture was diluted with water (5 mL) and extracted with ethyl acetate (3×30 mL). The organic phase was washed with brine (3×30 mL) and dried over sodium sulfate, filtered and concentrated under reduced pressure to give the residue. The residue was purified by flash silica gel chromatography (ISCO®; 12 g SepaFlash® Silica Flash Column, Eluent of 0˜60% Ethyl acetate/Petroleum ether gradient @40 mL/min) to give the title compound (575 mg, 87% yield) as a yellow solid. ¹H NMR (400 MHz, DMSO-d₆) δ=2.08 (d, J=4.8 Hz, 3H) 2.21-2.40 (m, 2H) 3.13 (br s, 3H) 3.25 (s, 2H) 3.60 (m, 2H) 3.82-3.91 (m, 6H) 4.30 (br s, 2H) 6.13 (br s, 1H) 6.82 (br s, 1H) 7.07 (br d, J=5.2 Hz, 1H) 7.14-7.31 (m, 1H) 7.72-7.77 (m, 1H) 12.55 (br s, 1H).

### 1-(5-(7-Fluoro-2-(4-(5-fluoro-2-methoxypyridin-3-yl)piperazine-1-carbonyl)-4-(4, 4,5,5-tetramethyl-1,3,2-dioxaborolan-2-yl)-1H-indol-6-yl)-3,6-dihydropyridin-1(2H)-yl)ethan-1-one (Intermediate MJ)

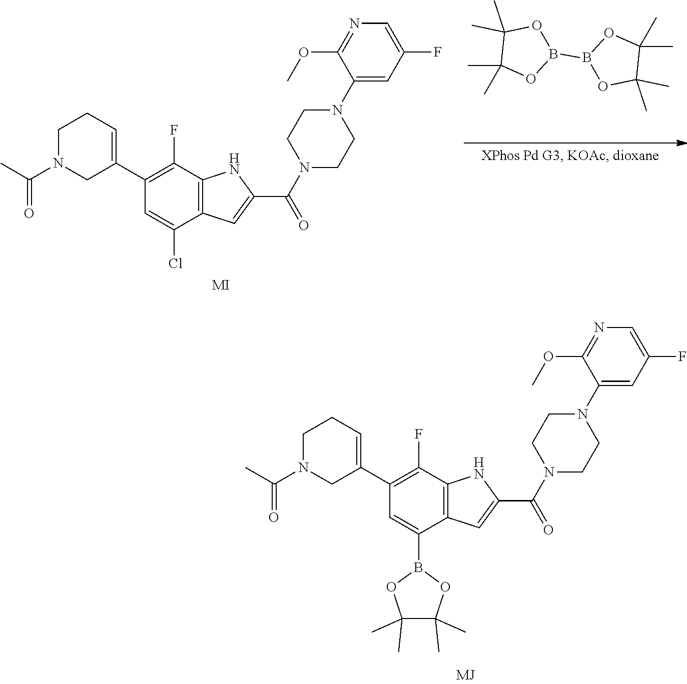

To a solution of 1-(5-(4-chloro-7-fluoro-2-(4-(5-fluoro-2-methoxypyridin-3-yl)piperazine-1-carbonyl)-1H-indol-6-yl)-3,6-dihydropyridin-1(2H)-yl)ethan-1-one (300 mg, 566 μmol, Intermediate MI) and 4,4,4′,4′,5,5,5′,5′-octamethyl-2,2′-bi(1,3,2-dioxaborolane) (215.62 mg, 849.11 μmol) in dioxane (3 mL) was added KOAc (166.67 mg, 1.70 mmol) and Xphos Pd G4 (48.71 mg, 56.61 μmol). The mixture was then stirred 90° C. at N₂ for 12 hrs. Upon completion, the mixture was diluted with water (10 mL) and extracted with ethyl acetate (3×30 mL). The organic phase was washed with brine (3×30 mL), dried over sodium sulfate, filtered and concentrated under reduced pressure to give the residue. The residue was purified by flash silica gel chromatography (ISCO®; 12 g SepaFlash® Silica Flash Column, Eluent of 0˜2% Dichloromethane:Methanol gradient @40 mL/min) to give the title compound (170 mg, 24% yield) as a yellow solid. ¹H NMR (400 MHz, DMSO-d₆) δ=1.33 (s, 12H) 2.54 (s, 3H) 3.11 (br s, 4H) 3.51-3.67 (m, 4H) 3.81 (br s, 4H) 3.87-3.90 (m, 3H) 3.95-4.08 (m, 2H) 4.29 (br s, 2H) 6.06 (br s, 1H) 7.00 (br d, J=1.2 Hz, 1H) 7.18-7.23 (m, 1H) 7.32-7.39 (m, 1H) 7.69-7.77 (m, 1H) 12.18 (s, 1H).

### 1-Bromo-2-ethyl-benzene (CAS #1973-22-4) (Intermediate MK)

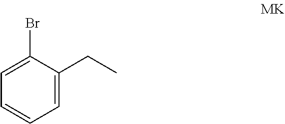

### 7-Fluoro-4-(4,4,5,5-tetramethyl-1,3,2-dioxaborolan-2-yl)-6-[1-[3-(triazol-1-yl)propanoyl]-3,6-dihydro-2H-pyridin-5-yl]-1H-indole-2-carboxylic acid (Intermediate ML)

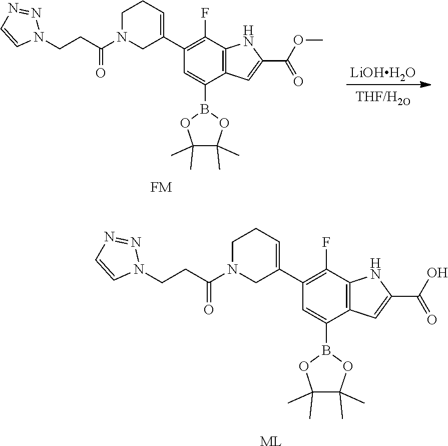

To a solution of methyl 7-fluoro-4-(4,4,5,5-tetramethyl-1,3,2-dioxaborolan-2-yl)-6-[1-[3-(triazol-1-yl)propanoyl]-3,6-dihydro-2H-pyridin-5-yl]-1H-indole-2-carboxylate (2 g, 3.82 mmol, Intermediate FM) in THF (10 mL) and H₂O (10 mL) was added LiOH·H₂O (801 mg, 19.1 mmol). The mixture was then stirred at 20° C. for 2 hrs. On completion, the mixture was acidified with HCl(2M) to adjust pH=4. During this period, brown precipitate was formed, which was filtered and the filter cake was collected to give the title compound (1.8 g, 74% yield) as a brown solid. LC-MS (ESI⁺) m/z 510.2 (M+H)⁺.

### 1-[5-[7-fluoro-2-(1-methyl-4,6-dihydropyrrolo[3,4-c]pyrazole-5-carbonyl)-4-(4,4,5,5-tetramethyl-1,3,2-dioxaborolan-2-yl)-1H-indol-6-yl]-3,6-dihydro-2H-pyridin-1-yl]-3-(triazol-1-yl)propan-1-one (Intermediate MM)

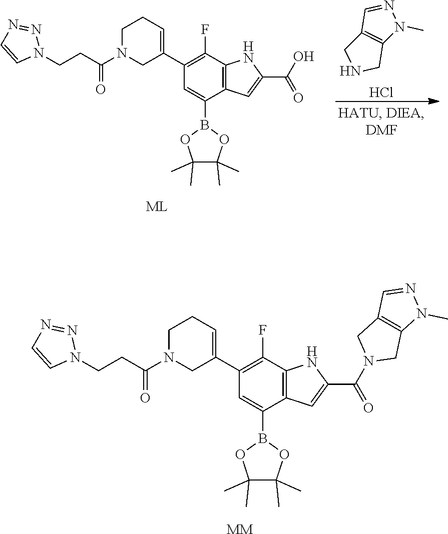

To a solution of 7-fluoro-4-(4,4,5,5-tetramethyl-1,3,2-dioxaborolan-2-yl)-6-[1-[3-(triazol-1-yl)propanoyl]-3,6-dihydro-2H-pyridin-5-yl]-1H-indole-2-carboxylic acid (1.5 g, 3.0 mmol, Intermediate ML), 1-methyl-5,6-dihydro-4H-pyrrolo[3,4-c]pyrazole (705.11 mg, 4.42 mmol, CAS #762233-62-5), HOBt (795.86 mg, 5.89 mmol) and DIEA (2.28 g, 17.7 mmol, 3.08 mL) in DMF (15 mL) was added HATU (1.34 g, 3.53 mmol). The mixture was then stirred at 20° C. for 0.5 hr. On completion, the reaction mixture was diluted with H₂O (20 mL) and extracted with EtOAc (30 mL×2). The combined organic layers were washed with brine (30 mL), dried over Na₂SO₄, filtered and concentrated under reduced pressure to give a residue. The residue was purified by flash silica gel chromatography (ISCO®; 12 g SepaFlash® Silica Flash Column, Eluent of 0-10% DCM/MeOH gradient @100 mL/min) to give the title compound (1.1 g, 55% yield) as a brown solid. LC-MS (ESI⁺) m/z 615.3 (M+H)⁺; ¹H NMR (400 MHz, CDCl₃-d) δ=8.03 (s, 1H), 7.78 (s, 1H), 7.68 (br d, J=5.6 Hz, 1H), 7.53 (br dd, J=6.8, 14.4 Hz, 1H), 7.43 (br s, 1H), 7.40-7.35 (m, 1H), 6.21-6.08 (m, 1H), 5.07-4.88 (m, 2H), 4.84-4.77 (m, 2H), 4.56 (t, J=5.2 Hz, 1H), 4.49-4.24 (m, 2H), 3.91 (s, 3H), 3.62-3.56 (m, 2H), 3.11-3.01 (m, 2H), 2.40-2.31 (m, 2H), 1.47-1.38 (m, 12H).

### 1-(3-(4-Chloro-7-fluoro-2-(1-methyl-1,4,5,6-tetrahydropyrrolo[3,4-c]pyrazole-5-carbonyl)-1H-indol-6-yl)-5,6-dihydropyridin-1(2H)-yl)ethanone (Intermediate MN)

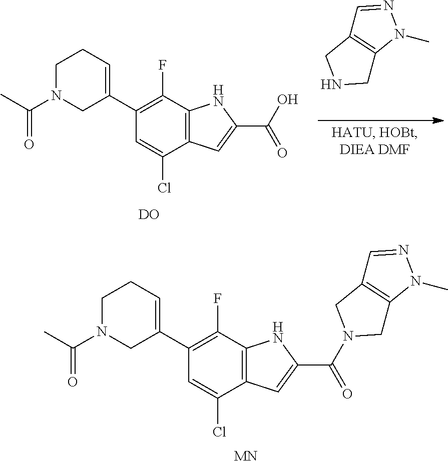

To a solution of 6-(1-acetyl-1,2,5,6-tetrahydropyridin-3-yl)-4-chloro-7-fluoro-1H-indole-2-carboxylic acid (1 g, 3 mmol, Intermediate DO) in DMF (10 mL) was added HOBt (601 mg, 4.45 mmol), HATU (1.35 g, 3.56 mmol), DIEA (1.92 g, 14.8 mmol, 2.59 mL) and 1-methyl-1,4,5,6-tetrahydropyrrolo[3,4-c]pyrazole (568 mg, 3.56 mmol, HCl, CAS #762233-62-5). The mixture was then stirred at 25° C. for 2 hrs. On completion, the reaction mixture was triturated with H₂O at 0° C. for 15 min, then filtered and dried to give the title compound (1.1 g) as a white solid. LC-MS (ESI⁺) m/z 442.0 (M+H)⁺.

### 1-(5-(7-Fluoro-2-(1-methyl-1,4,5,6-tetrahydropyrrolo[3,4-c]pyrazole-5-carbonyl)-4-(4,4,5,5-tetramethyl-1,3,2-dioxaborolan-2-yl)-1H-indol-6-yl)-3,6-dihydropyridin-1(2H)-yl)ethan-1-one (Intermediate MO)

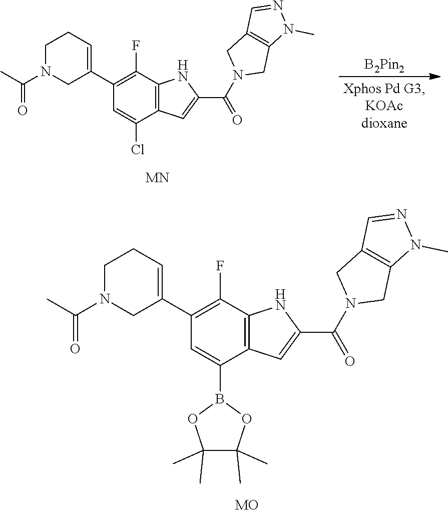

To a solution of 1-(5-(4-chloro-7-fluoro-2-(1-methyl-1,4,5,6-tetrahydropyrrolo[3,4-c]pyrazole-5-carbonyl)-1H-indol-6-yl)-3,6-dihydropyridin-1(2H)-yl)ethan-1-one (500 mg, 1.13 mmol, Intermediate MN) and 4,4,5,5-tetramethyl-2-(4,4,5,5-tetramethyl-1,3,2-dioxaborolan-2-yl)-1,3,2-dioxaborolane (575 mg, 2.26 mmol) in dioxane (10 mL) was added XPhos Pd G3 (95.8 mg, 113 μmol) and KOAc (333 mg, 3.39 mmol). The mixture was then stirred at 80° C. for 2 hrs. On completion, was concentrated under reduced pressure to remove solvent. Then the mixture was purified by column chromatography (SiO₂, DCM:MeOH=10/1) to give the title compound (240 mg, 34% yield) as a brown solid. LC-MS (ESI⁺) m/z 534.2 (M+H)⁺.

### 4-Chloro-3-isopropyl-pyridine (CAS #860412-09-5) (Intermediate MP)

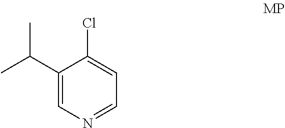

### 1-[5-[4-Chloro-7-fluoro-2-[4-(5-fluoro-3-methoxy-2-pyridyl)piperazine-1-carbonyl]-1H-indol-6-yl]-3,6-dihydro-2H-pyridin-1-yl]ethanone (Intermediate MQ)

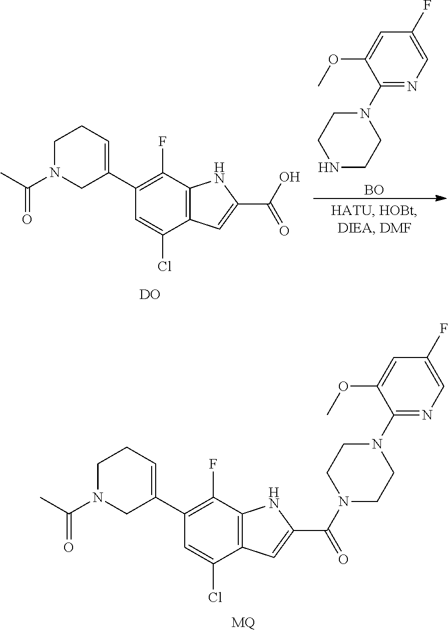

To a solution of 6-(1-acetyl-3,6-dihydro-2H-pyridin-5-yl)-4-chloro-7-fluoro-1H-indole-2-carboxylic acid (500 mg, 1.48 mmol, Intermediate DO) in DMF (10 mL) was added HATU (677.48 mg, 1.78 mmol), HOBt (401.26 mg, 2.97 mmol), and DIEA (959.50 mg, 7.42 mmol, 1.29 mL). Then 1-(5-fluoro-3-methoxy-2-pyridyl)piperazine (313.64 mg, 1.48 mmol, Intermediate BO) was added into the solution. The mixture was stirred at 20° C. for 0.5 hr. Upon completion, the mixture was diluted with ethyl acetate (20 mL) and water (20 mL). The mixture was extracted with ethyl acetate (3×20 mL). The combined organic extracts were washed with brine (20 mL) and then dried over anhydrous sodium sulfate, filtered and concentrated under vacuum and purified by flash silica gel chromatography (ISCO®; 12 g SepaFlash® Silica Flash Column, Eluent of 0˜5% MeOH/DCM gradient @60 mL/min) to give the title compound (520 mg, 60% yield) as a yellow solid. ¹H NMR (400 MHz, DMSO-d₆) δ=12.52 (br s, 1H), 7.80 (d, J=2.4 Hz, 1H), 7.38 (dd, J=2.4, 10.4 Hz, 1H), 7.21-7.00 (m, 1H), 6.80 (br s, 1H), 6.13 (br s, 1H), 4.35-4.24 (m, 2H), 3.85 (s, 3H), 3.84-3.75 (m, 4H), 3.65-3.54 (m, 2H), 3.31-3.25 (m, 4H), 2.39-2.23 (m, 2H), 2.07 (br d, J=5.6 Hz, 3H).

### 1-[5-[7-fluoro-2-[4-(5-fluoro-3-methoxy-2-pyridyl)piperazine-1-carbonyl]-4-(4,4,5,5-tetramethyl-1,3,2-dioxaborolan-2-yl)-1H-indol-6-yl]-3,6-dihydro-2H-pyridin-1-yl]ethanone (Intermediate

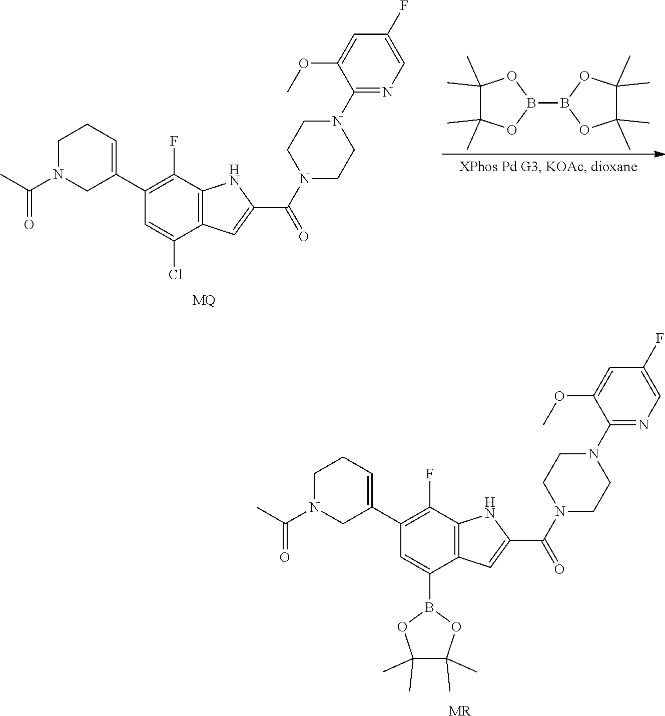

A mixture of 1-[5-[4-chloro-7-fluoro-2-[4-(5-fluoro-3-methoxy-2-pyridyl)piperazine-1-carbonyl]-1H-indol-6-yl]-3,6-dihydro-2H-pyridin-1-yl]ethanone (450 mg, 849.11 μmol, Intermediate MQ), 4,4,5,5-tetramethyl-2-(4,4,5,5-tetramethyl-1,3,2-dioxaborolan-2-yl)-1,3,2-dioxaborolane (258.75 mg, 1.02 mmol), XPhos Pd G3 (71.87 mg, 84.91 μmol), and KOAc (250.00 mg, 2.55 mmol) in dioxane (10 mL) was degassed and purged with N₂ three times. Then the mixture was stirred at 90° C. for 12 hrs under N₂ atmosphere. On completion, the mixture was diluted with ethyl acetate (20 mL) and water (20 mL). The mixture was extracted with ethyl acetate (3×20 mL). The combined organic extracts were washed with brine (20 mL) and then dried over anhydrous sodium sulfate, filtered and concentrated under vacuum to give a residue. The residue was purified by flash silica gel chromatography (ISCO®; 20 g SepaFlash® Silica Flash Column, Eluent of 0˜5% MeOH/DCM gradient @40 mL/min) to give the title compound (520 mg, 53% yield) as a yellow solid. LC-MS (ESI+) m/z 622.3 (M+H)⁺.

### 1-Methyl-1,4,5,6-tetrahydropyrrolo[3,4-c]pyrazole (CAS #762233-62-5) (Intermediate MIS)

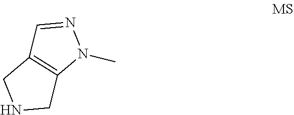

### 3-Bromo-4-methoxypyridine (CAS #82257-09-8) (Intermediate MT)

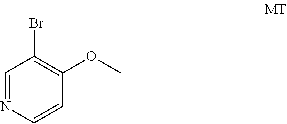

### (R)-1-(3-(4-chloro-7-fluoro-2-(1-methyl-1,4,5,6-tetrahydropyrrolo[3,4-c]pyrazole-5-carbonyl)-1H-indol-6-yl)piperidin-1-yl)-3-(1H-1,2,3-triazol-1-yl)propan-1-one (Intermediate MU)

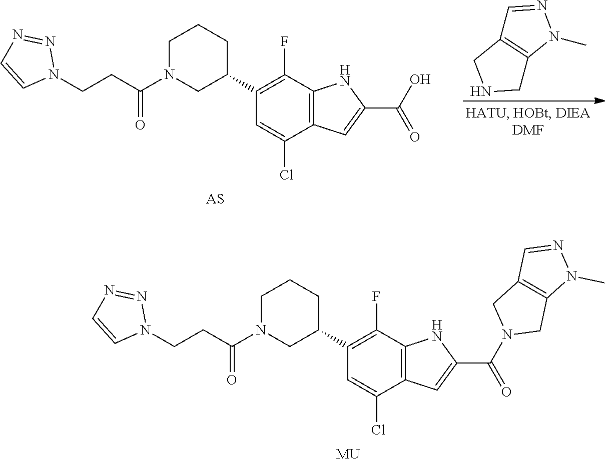

A mixture of (R)-6-(1-(3-(1H-1,2,3-triazol-1-yl)propanoyl)piperidin-3-yl)-4-chloro-7-fluoro-1H-indole-2-carboxylic acid (140 mg, 333 μmol, Intermediate AS), 1-methyl-1,4,5,6-tetrahydropyrrolo[3,4-c]pyrazole (63.8 mg, 400 μmol, HCl, CAS #762233-62-5), HATU (152 mg, 400 μmol), DIEA (172 mg, 1.33 mmol, 232 μL) and HOBt (54.0 mg, 400 μmol) in DMF (2 mL) was stirred at 25° C. for 10 min. Upon completion, the crude product was triturated with water at 25° C. for 0.5 hrs, then the mixture was filtered and the filter cake was concentrated dried in vacuo to give the title compound (130 mg, 64% yield) as a yellow solid. ¹H NMR (400 MHz, DMSO-d₆) δ=12.36 (s, 1H), 8.13-8.06 (m, 1H), 7.69 (d, J=9.6 Hz, 1H), 7.29 (d, J=10.8 Hz, 1H), 7.19-7.14 (m, 1H), 7.11 (d, J=11.6 Hz, 1H), 5.12 (s, 1H), 5.00-4.92 (m, 1H), 4.80 (s, 1H), 4.73-4.52 (m, 4H), 4.46 (t, J=14.8 Hz, 1H), 3.93-3.85 (m, 1H), 3.82 (s, 3H), 3.28-3.19 (m, 1H), 3.11-2.97 (m, 4H), 1.91-1.85 (m, 2H), 1.80-1.73 (m, 1H).

### (R)-1-(3-(7-fluoro-2-(1-methyl-1,4,5,6-tetrahydropyrrolo[3,4-c]pyrazole-5-carbonyl)-4-(4,4,5,5-tetramethyl-1,3,2-dioxaborolan-2-yl)-1H-indol-6-yl)piperidin-1-yl)-3-(1H-1,2,3-triazol-1-yl)propan-1-one (Intermediate MV)

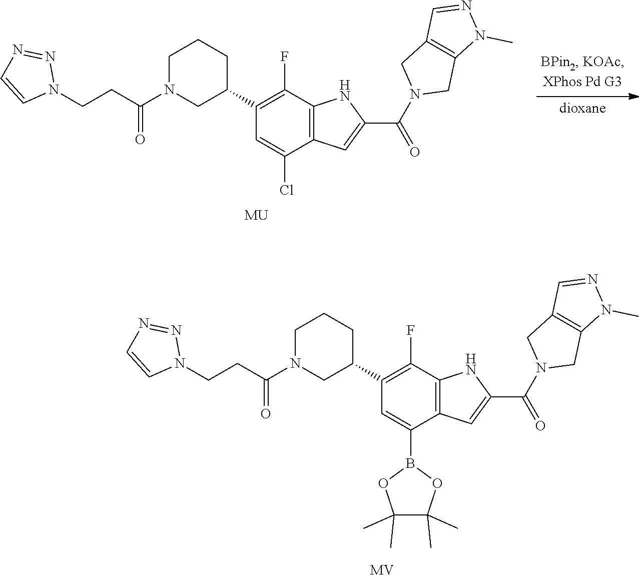

A mixture of (R)-1-(3-(4-chloro-7-fluoro-2-(1-methyl-1,4,5,6-tetrahydropyrrolo[3,4-c]pyrazole-5-carbonyl)-1H-indol-6-yl)piperidin-1-yl)-3-(1H-1,2,3-triazol-1-yl)propan-1-one (130 mg, 247 mol, Intermediate MU), 4,4,5,5-tetramethyl-2-(4,4,5,5-tetramethyl-1,3,2-dioxaborolan-2-yl)-1,3,2-dioxaborolane (188 mg, 742 μmol), KOAc (72.9 mg, 742 μmol), and XPhos Pd G3 (20.9 mg, 24.7 μmol) in dioxane (2 mL) was degassed and purged with N₂ three times. Then the mixture was stirred at 90° C. under N2 atmosphere for 2 hrs. On completion, the reaction mixture was quenched with water (10 mL) and extracted with ethyl acetate (4×10 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=100/1 to 0/1 then with SiO₂, Dichloromethane:Methanol=10/1) to give the tittle compound (80.0 mg, 41% yield) as a yellow solid. LC-MS (ESI⁺)/z 617.3 (M+H)⁺.

### (R)-methyl 6-(1-(3-(1H-1,2,3-triazol-1-yl)propanoyl)piperidin-3-yl)-7-fluoro-4-(4,4,5,5-tetramethyl-1,3,2-dioxaborolan-2-yl)-1H-indole-2-carboxylate (Intermediate MW)

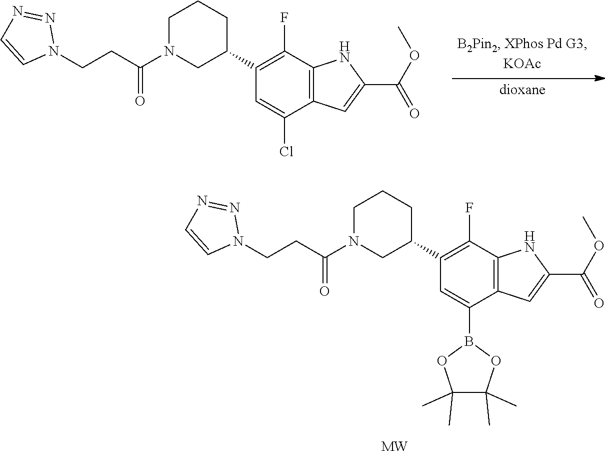

A mixture of (R)-methyl 6-(1-(3-(1H-1,2,3-triazol-1-yl)propanoyl)piperidin-3-yl)-4-chloro-7-fluoro-1H-indole-2-carboxylate (200 mg, 460 μmol, synthesized via Step 1 of Intermediate AS), 4,4,5,5-tetramethyl-2-(4,4,5,5-tetramethyl-1,3,2-dioxaborolan-2-yl)-1,3,2-dioxaborolane (468 mg, 1.84 mmol), KOAc (135 mg, 1.38 mmol), and XPhos Pd G3 (39.0 mg, 46.1 μmol) in dioxane (4 mL) was degassed and purged with N₂ three times. Then the mixture was stirred at 90° C. for 2 hrs under N₂ atmosphere. Upon completion, the crude product was triturated with water at 25° C. for 1 hr, then the mixture was filtered and the filter cake was dried in vacuo to give the title compound (150 mg) as a yellow solid. LC-MS (ESI⁺)/z 526.3 (M+H)⁺.

### (R)-6-(1-(3-(1H-1,2,3-triazol-1-yl)propanoyl)piperidin-3-yl)-4-(4-ethylpyridin-3-yl)-7-fluoro-1H-indole-2-carboxylic acid (Intermediate MX)

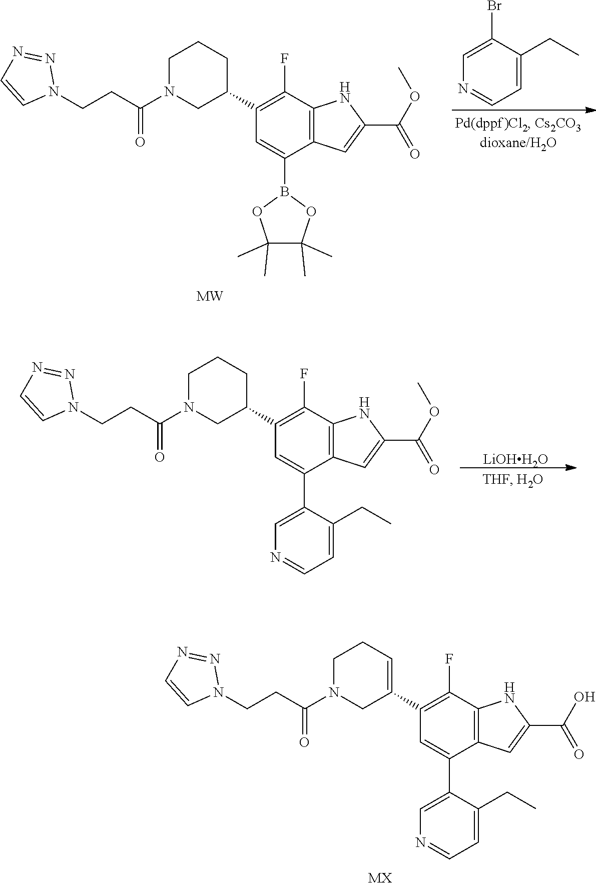

### Step 1—(R)-methyl 6-(1-(3-(1H-1,2,3-triazol-1-yl)propanoyl)piperidin-3-yl)-4-(4-ethylpyridin-3-yl)-7-fluoro-1H-indole-2-carboxylate

A mixture of (R)-methyl 6-(1-(3-(1H-1,2,3-triazol-1-yl)propanoyl)piperidin-3-yl)-7-fluoro-4-(4,4,5,5-tetramethyl-1,3,2-dioxaborolan-2-yl)-1H-indole-2-carboxylate (155 mg, 295 μmol, Intermediate MW), 3-bromo-4-ethylpyridine (50.0 mg, 268 μmol, CAS #38749-76-7), Cs₂CO₃ (262 mg, 806 μmol), and Pd(dppf)Cl₂·CH₂Cl₂ (21.9 mg, 26.8 μmol) in dioxane (2 mL) and H₂O (0.5 mL) was degassed and purged with N₂ three times. Then the mixture was stirred at 80° C. for 2 hours under N₂ atmosphere. On completion, the reaction mixture was quenched with water (10 mL) and extracted with ethyl acetate (3×20 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=100/1 to 0/1) and (SiO₂, Dichloromethane:Methanol=10/1) to give the tittle compound (120 mg, 89 yield) as a yellow solid. ¹H NMR (400 MHz, DMSO-d₆) δ=12.74-12.54 (m, 1H), 8.55 (d, J=5.2 Hz, 1H), 8.38 (d, J=5.2 Hz, 1H), 8.14-8.08 (m, 1H), 7.69 (d, J=8.8 Hz, 1H), 7.46-7.40 (m, 1H), 7.06-6.99 (m, 1H), 6.68 (t, J=2.4 Hz, 1H), 4.66-4.55 (m, 2H), 4.48 (d, J=13.2 Hz, 1H), 3.96-3.87 (m, 1H), 3.84 (s, 3H), 3.29-3.23 (m, 1H), 3.20-3.20 (m, 1H), 3.08-2.99 (m, 3H), 2.46 (s, 3H), 1.96-1.81 (m, 2H), 1.80-1.69 (m, 1H), 1.27-1.21 (m, 1H), 0.98 (dt, J=3.6, 7.6 Hz, 3H).

### Step 2—(R)-6-(1-(3-(1H-1,2,3-triazol-1-yl)propanoyl)piperidin-3-yl)-4-(4-ethylpyridin-3-yl)-7-fluoro-1H-indole-2-carboxylic acid

A mixture of (R)-methyl 6-(1-(3-(1H-1,2,3-triazol-1-yl)propanoyl)piperidin-3-yl)-4-(4-ethylpyridin-3-yl)-7-fluoro-1H-indole-2-carboxylate (120 mg, 237 μmol), and LiOH·H₂O (49.9 mg, 1.19 mmol) in THF (2 mL) and H₂O (2 mL) was stirred at 40° C. for 2 hrs. On completion, the crude product was triturated with water at 25° C. for 0.5 hrs, then the mixture was filtered and the filter cake was dried in vacuo to give the title compound (60.0 mg, 41% yield) as a white solid. LC-MS (ESI⁺)/z 491.2 (M+H)⁺.

### 2-Chloro-5-fluoro-3-methoxy-6-methylpyridine (Intermediate MY)

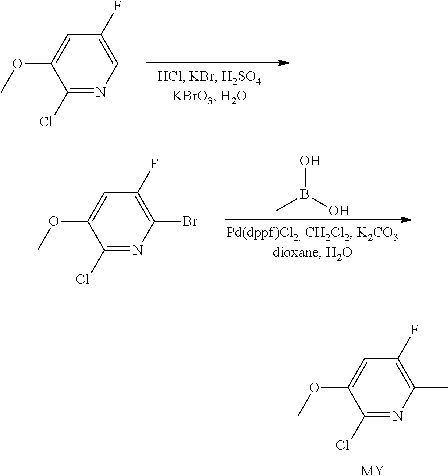

### Step 1—2-Bromo-6-chloro-3-fluoro-5-methoxypyridine

2-chloro-5-fluoro-3-methoxy-pyridine (1 g, 6 mmol, CAS #1097264-89-5) and HCl (1 M, 8.11 mL) was added to a 40 ml vial. Then KBr (4.70 g, 39.4 mmol) and H₂SO₄ (3 M, 16.2 mL) was also added to the vial. The mixture was then added to a 250 mL flask which contained KBrO₃ (1.29 g, 7.74 mmol) and H₂O (60 mL). Then the mixture was stirred at 25° C. for 10 mins. On completion, Na₂SO₃ aq. (1.3 M, 50 mL) was added and the mixture was stirred for 5 mins, and the mixture was extracted with DCM (30 mL×3). The combined organic layers were dried over Na₂SO₄, filtered and concentrated under reduced pressure to give a residue. The crude product was triturated with PE (20 mL) to afford the title compound (1.49 g, 97% yield) as a yellow solid. LC-MS (ESI⁺) m/z 239.9 (M+H)⁺; ¹H NMR (400 MHz, DMSO-d₆) δ=7.90 (d, J=9.2 Hz, 1H), 3.93 (s, 3H).

### Step 2—2-Chloro-5-fluoro-3-methoxy-6-methylpyridine

A mixture of 2-bromo-6-chloro-3-fluoro-5-methoxy-pyridine (800 mg, 3.33 mmol), methylboronic acid (219 mg, 3.66 mmol, CAS #13061-96-6), Pd(dppf)Cl₂·CH₂Cl₂ (271 mg, 332 μmol) and K₂CO₃ (1.38 g, 9.98 mmol) in dioxane (8 mL) and H₂O (2 mL) was degassed and purged with N₂ three times. Then the mixture was stirred at 80° C. for 1 hr under N₂ atmosphere. The reaction mixture was filtered and concentrated under reduced pressure to give a residue. The residue was purified by column chromatography (SiO₂, Petroleum ether/Ethyl acetate=50/1 to 6/1) to afford the title compound (520 mg, 78% yield) as a white solid. LC-MS (ESI⁺) m/z 176.1 (M+H)⁺.

### 1-(5-Fluoro-3-methoxy-6-methylpyridin-2-yl)piperazine (Intermediate MZ)

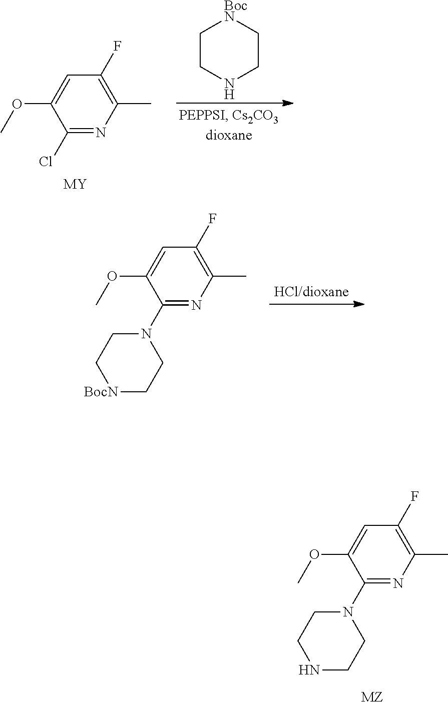

### Step 1—Tert-butyl 4-(5-fluoro-3-methoxy-6-methylpyridin-2-yl)piperazine-1-carboxylate

A mixture of 2-chloro-5-fluoro-3-methoxy-6-methyl-pyridine (300 mg, 1.71 mmol, Intermediate MY), tert-butyl piperazine-1-carboxylate (318 mg, 1.71 mmol, CAS #57260-71-6), 1,3-bis[2,6-bis(1-propylbutyl)phenyl]-4,5-dichloro-2H-imidazol-1-ium-2-ide;3-chloropyridine dichloropalladium (166 mg, 170 μmol) and Cs₂CO₃ (1.67 g, 5.13 mmol) in dioxane (6 mL) was degassed and purged with N₂ three times. Then the mixture was stirred at 100° C. for 1 hr under N₂ atmosphere. The reaction mixture was filtered and concentrated under reduced pressure to give a residue. The residue was purified by column chromatography (SiO₂, Petroleum ether/Ethyl acetate=50/1 to 10/1) to afford the title compound (450 mg, 54% yield) as a yellow solid. LC-MS (ESI⁺) m/z 326.1 (M+H)⁺.

### Step 2—1-(5-Fluoro-3-methoxy-6-methylpyridin-2-yl)piperazine

To a solution of tert-butyl 4-(5-fluoro-3-methoxy-6-methyl-2-pyridyl)piperazine-1-carboxylate (450 mg, 1.38 mmol) in HCl/dioxane (2 M, 5 mL) and the mixture was stirred at 25° C. for 1 hr. On completion, the reaction mixture was concentrated under reduced pressure to afford the title compound (300 mg) as a yellow solid. LC-MS (ESI⁺) m/z 226.1 (M+H)⁺; ¹H NMR (400 MHz, DMSO-d₆) δ=7.34 (d, J=10.4 Hz, 1H), 3.80 (s, 3H), 3.56 (s, 1H), 3.44-3.40 (m, 4H), 3.16 (s, 4H), 2.27 (d, J=3.2 Hz, 3H).

### 5-Chloro-4-ethyl-2-methylpyridine (Intermediate NA)

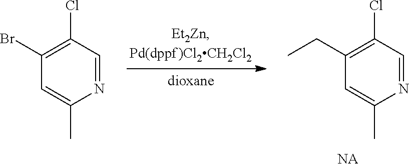

A mixture of 4-bromo-5-chloro-2-methylpyridine (1.00 g, 4.84 mmol, CAS #1211529-34-8) and Pd(dppf)Cl₂·CH₂Cl₂ (395 mg, 484 μmol) in dioxane (8.00 mL) was degassed and purged with N₂ three times. Then ZnEt₂ (1 M, 2.42 mL) was added and then the mixture was stirred at 70° C. for 3 hrs under N₂ atmosphere. On completion, the reaction mixture was quenched by addition of H₂O (5.00 mL) at 0° C., and then extracted with EA (5.00 mL×2). The combined organic layers were washed with NaCl (5.00 mL×3), dried over Na₂SO₄, filtered and concentrated under reduced pressure to give a residue. The crude product was purified by reversed-phase HPLC (0.1% FA condition) and then extracted with EA (5.00 mL×2). The combined organic layers were washed with NaCl 15.0 mL (5.00 mL×3), dried over Na₂SO₄, filtered and concentrated under reduced pressure to give the title compound (490 mg, 60% yield) as a black oil. LC-MS (ESI⁺) m/z 156.2 (M+H)⁺; ¹H NMR (400 MHz, CDCl³-d) δ=8.40 (s, 1H), 7.03 (s, 1H), 2.76 (q, J=7.6 Hz, 2H), 2.36 (s, 3H), 1.28 (t, J=7.6 Hz, 3H).

### 1-(3,3-Difluorocyclobutyl)piperazine (Intermediate NB)

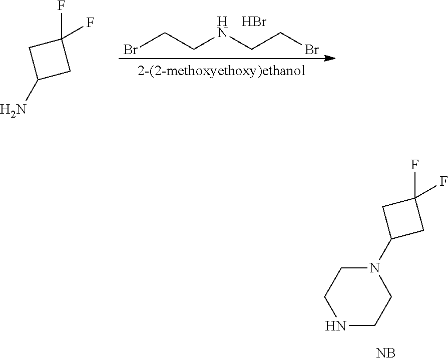

To a solution of 3,3-difluorocyclobutanamine (200 mg, 1.87 mmol, CAS #791061-00-2) in 2-(2-methoxyethoxy)ethanol (6 mL) was added 2-bromo-N-(2-bromoethyl)ethanamine (1.16 g, 3.73 mmol, HBr, CAS #3890-99-1). The mixture was then stirred at 130° C. for 3 h. On completion, the reaction mixture was concentrated under reduced pressure to give a residue. The residue was purified by prep-HPLC (column: CD21-PN-C18 250*30*5 um;mobile phase: [water(NH₄HCO₃)-ACN]; gradient:0%-30% B over 15 min) to give the title compound (400 mg) as a yellow gum. LC-MS (ESI⁺) m/z 177.2 (M+H)⁺.

### [6-Chloro-4-(4-ethyl-3-pyridyl)-7-fluoro-1H-indol-2-yl]-[4-(5-fluoro-3-methoxy-2-pyridyl)piperazin-1-yl]methanone (Intermediate NC)

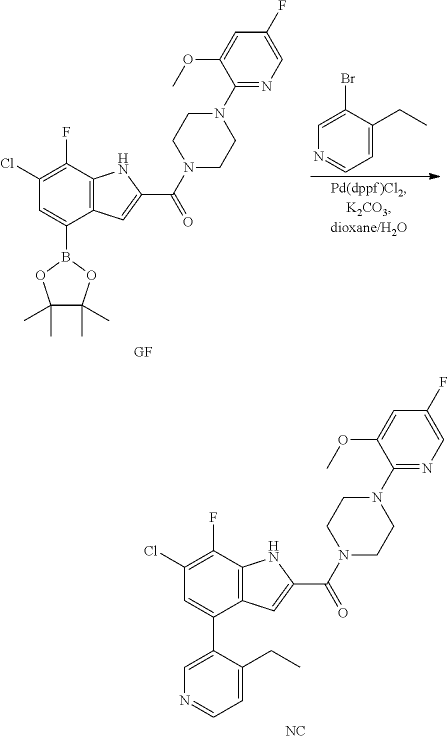

A mixture of [6-chloro-7-fluoro-4-(4,4,5,5-tetramethyl-1,3,2-dioxaborolan-2-yl)-1H-indol-2-yl]-[4-(5-fluoro-3-methoxy-2-pyridyl)piperazin-1-yl]methanone (280 mg, 526 μmol, Intermediate GF), 3-bromo-4-ethyl-pyridine (146.67 mg, 788.33 μmol, CAS #38749-76-7), Pd(dppf)Cl₂ (38.45 mg, 52.56 μmol), and K₂CO₃ (217.90 mg, 1.58 mmol) in dioxane (3 mL) and H₂O (0.5 mL) was degassed and purged with N₂ three times. Then the mixture was stirred at 80° C. for 2 hrs under N₂ atmosphere. On completion, the mixture was diluted with ethyl acetate (20 mL) and water (20 mL), and the mixture was extracted with ethyl acetate (3×20 mL). The combined organic extracts were washed brine (20 mL) and then dried over anhydrous sodium sulfate, filtered and concentrated under vacuum to give a residue. The residue was purified by flash silica gel chromatography (ISCO® R; 12 g SepaFlash® Silica Flash Column, Eluent of 0˜0.5% MeOH/DCM gradient @40 mL/min) and by prep-TLC (SiO₂, DCM/MeOH=10/1) to give the title compound (50 mg, 12% yield) as yellow solid. LC-MS (ESI+) m/z 512.2 (M+H)⁺.

### 4-[Tert-butyl(dimethyl)silyl]oxybutanoic acid (Intermediate ND)

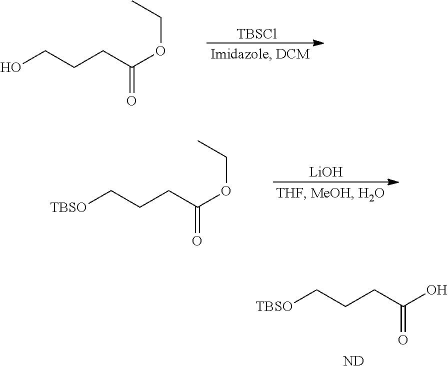

### Step 1—Ethyl 4-[tert-butyl(dimethyl)silyl]oxybutanoate

To a solution of ethyl 4-hydroxybutanoate (1 g, 8 mmol, CAS #999-10-0) in DCM (10 mL) was added imidazole (1.55 g, 22.70 mmol) and TBSCl (1.71 g, 11.35 mmol, 1.40 mL). The mixture was then stirred at 25° C. for 12 hours. On completion, the reaction mixture was quenched by addition of H₂O (10 mL) at 0° C., and then extracted with ethyl acetate (3×20 mL). The combined organic layers were dried over Na₂SO₄, filtered and concentrated under reduced pressure to give the title compound (2 g) as a white solid. H NMR (400 MHz, DMSO-d6) 4.04 (q, J=7.2 Hz, 2H), 3.58 (t, J=6.4 Hz, 2H), 2.31 (t, J=7.2 Hz, 2H), 1.74-1.66 (m, 2H), 1.17 (t, J=7.2 Hz, 3H), 0.86-0.84 (m, 9H), 0.02-0.01 (m, 6H).

### Step 2—4-[Tert-butyl(dimethyl)silyl]oxybutanoic acid

To a solution of ethyl 4-[tert-butyl(dimethyl)silyl]oxybutanoate (1 g, 4 mmol) in THF (8 mL), MeOH (2 mL) and H₂O (2 mL) was added LiOH·H₂O (425.74 mg, 10.15 mmol). The mixture was then stirred at 25° C. for 12 hrs. On completion, the reaction mixture was filtered and concentrated under reduced pressure to give the title compound (1 g) as a white solid. ¹H NMR (400 MHz, CDCl₃-d) δ 3.58 (br s, 2H), 2.17 (br s, 2H), 1.74 (br s, 2H), 0.88 (s, 9H), 0.04 (s, 6H).

### 4-[Tert-butyl(dimethyl)silyl]oxy-1-[5-(4,4,5,5-tetramethyl-1,3,2-dioxaborolan-2-yl)-3,6-dihydro-2H-pyridin-1-yl]butan-1-one (Intermediate NE)

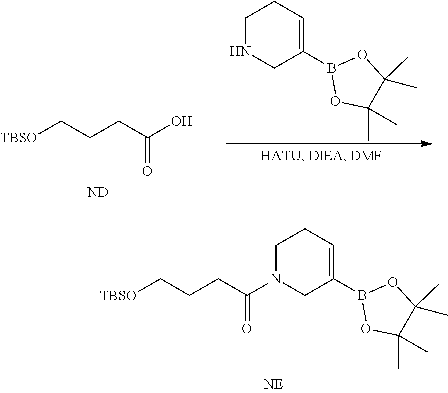 [SMILES: CC(C)(C)[Si](C)(C)OCCCC(=O)O ; CC1(C)OB(C2=CCCNC2)OC1(C)C ; "ND" ; CC1(C)OB(C2=CCCN(C(=O)CCCO[Si](C)(C)C(C)(C)C)C2)OC1(C)C]

To a solution of 4-[tert-butyl(dimethyl)silyl]oxybutanoic acid (300 mg, 1.37 mmol, Intermediate ND) in DMF (5 mL) was added DIEA (888 mg, 6.87 mmol, 1.20 mL) and HATU (626.85 mg, 1.65 mmol) at 0° C. sand the mixture was stirred for 0.5 hr. Then 5-(4,4,5,5-tetramethyl-1,3,2-dioxaborolan-2-yl)-1,2,3,6-tetrahydropyridine (337.35 mg, 1.37 mmol, HCl, synthesized via Step 1 of Intermediate U) was added into the solution. The mixture was stirred at 20° C. for another 0.5 hr. On completion, the mixture was diluted with ethyl acetate (20 mL) and water (20 mL) then was extracted with ethyl acetate (3×20 mL). The combined organic extracts were washed with brine (20 mL), dried over anhydrous sodium sulfate, filtered and concentrated under vacuum. The residue was purified by flash silica gel chromatography (ISCO®; 20 g SepaFlash® Silica Flash Column, Eluent of 0˜20% Ethyl acetate/Petroleum ether gradient @80 mL/min) to give title compound (50 mg, 8.62% yield) as a colorless oil. ¹H NMR (400 MHz, CDCl₃-d) δ 6.75-6.57 (m, 1H), 4.17 (br d, J=2.0 Hz, 1H), 4.03 (br d, J=2.0 Hz, 1H), 3.71-3.63 (m, 3H), 3.51 (t, J=5.6 Hz, 1H), 2.48-2.38 (m, 2H), 2.22 (br d, J=2.0 Hz, 2H), 1.86 (quin, J=6.8 Hz, 2H), 1.28-1.24 (m, 12H), 0.89 (s, 9H), 0.05 (s, 6H).

### Methyl 5-(4,4,5,5-tetramethyl-1,3,2-dioxaborolan-2-yl)-3,6-dihydropyridine-1(2H)-carboxylate (Intermediate NF)

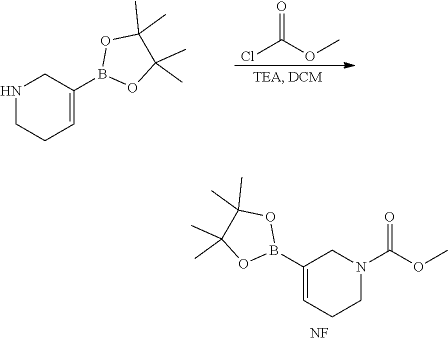

To a solution of 5-(4,4,5,5-tetramethyl-1,3,2-dioxaborolan-2-yl)-1,2,3,6-tetrahydropyridine (500 mg, 2.39 mmol) in DCM (10 mL) was added TEA (483 mg, 4.78 mmol, 665 μL) and the mixture was degassed and purged with N₂ three times and cooled to 0° C. Then methyl carbonochloridate (271 mg, 2.87 mmol, 221 μL, CAS #79-22-1) was added to the mixture at 0° C. and the mixture was stirred at 20° C. for 1 hr under N₂ atmosphere. On completion, the reaction was quenched with H₂O (20 mL) and DCM (20 mL) and H₂O (20 mL) were added and layers were separated. The aqueous phase was extracted with DCM (20 mL×3). The combined organic extract was dried over Na₂SO₄, filtered, and concentrated under vacuum to give a residue. The residue was purified by flash silica gel chromatography (ISCO®; 20 g SepaFlash® Silica Flash Column, Eluent of 0˜15% Ethyl acetate/Petroleum ether gradient @80 mL/min) to give the title compound (400 mg, 63% yield) as a white solid. ¹H NMR (400 MHz, DMSO-d₆) δ=6.53 (br s, 1H) 3.87 (br d, J=1.2 Hz, 2H) 3.60 (s, 3H) 3.41 (br t, J=5.6 Hz, 2H) 2.15 (m, 2H) 1.21 (s, 12H).

### 7-Fluoro-4-(3-methoxy-4-pyridyl)-6-[1-[3-(triazol-1-yl)propanoyl]-3,6-dihydro-2H-pyridin-5-yl]-1H-indole-2-carboxylic acid (Intermediate NG)

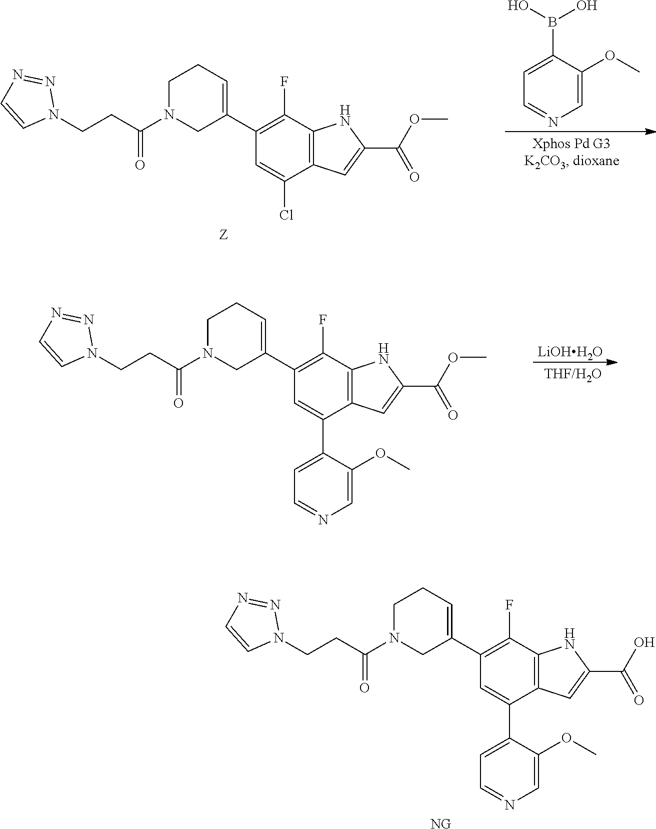

### Step 1—Methyl 7-fluoro-4-(3-methoxy-4-pyridyl)-6-[1-[3-(triazol-1-yl)propanoyl]-3,6-dihydro-2H-pyridin-5-yl]-1H-indole-2-carboxylate

A mixture of methyl 4-chloro-7-fluoro-6-[1-[3-(triazol-1-yl)propanoyl]-3,6-dihydro-2H-pyridin-5-yl]-1H-indole-2-carboxylate (400 mg, 926 μmol, Intermediate Z), (3-methoxy-4-pyridyl)boronic acid (141 mg, 926 μmol, CAS #1008506-24-8), XPhos Pd G₃ (78.4 mg, 92.6 μmol), and K₂CO₃ (384 mg, 2.78 mmol) in dioxane (2 mL) and H₂O (0.4 mL) was degassed and purged with N₂ three times. Then the mixture was stirred at 80° C. for 1 hr under N₂ atmosphere. On completion, the reaction mixture was diluted with H₂O (3 mL) and extracted with EtOAc (8 mL×2). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 0˜10% DCM/MeOH gradient @100 mL/min) to give the title compound (200 mg, 34% yield) as a brown solid. LC-MS (ESI⁺) m/z 505.2 (M+H)⁺.

### Step 2—7-Fluoro-4-(3-methoxy-4-pyridyl)-6-[1-[3-(triazol-1-yl)propanoyl]-3,6-dihydro-2H-pyridin-5-yl]-1H-indole-2-carboxylic acid

To a solution of methyl 7-fluoro-4-(3-methoxy-4-pyridyl)-6-[1-[3-(triazol-1-yl)propanoyl]-3,6-dihydro-2H-pyridin-5-yl]-1H-indole-2-carboxylate (200 mg, 396 μmol) in THE (1 mL) and H₂O (1 mL) was added LiOH·H₂O (83.1 mg, 1.98 mmol). The mixture was then stirred at 20° C. for 3 hr. On completion, the mixture was acidified with HCl (2M) to adjust pH=4. During this period, brown precipitate was formed, which was filtered and the filter cake was collected to give the title compound (150 mg) as a brown solid. LC-MS (ESI⁺) m/z 491.2 (M+H)⁺.

### 1-Ethyl-1,4,5,6-tetrahydropyrrolo[3,4-c]pyrazole (Intermediate NH) and 2-ethyl-2,45,6-tetrahydropyrrolo[3,4-c]pyrazole (Intermediate NI)

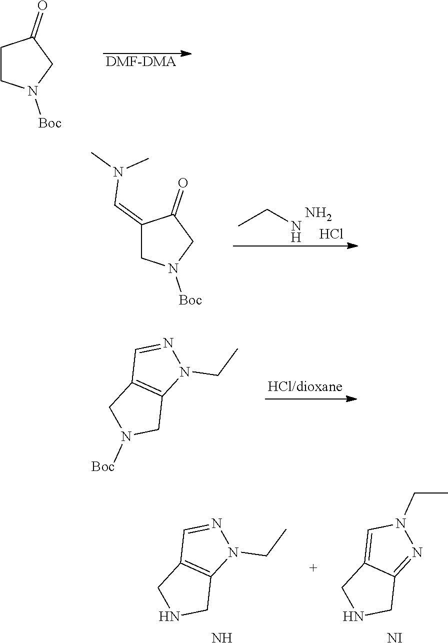

### Step 1—(Z)-tert-butyl 3-((dimethylamino)methylene)-4-oxopyrrolidine-1-carboxylate

A solution of tert-butyl 3-oxopyrrolidine-1-carboxylate (5 g, 30 mmol, CAS #101385-93-7) in DMF-DMA (20 mL) was stirred at 105° C. for 1 hr under N₂ atmosphere. The reaction mixture was filtered and the filtrate concentrated under reduced pressure to give the title compound (4.88 g) as a yellow solid. ¹H NMR (400 MHz, DMSO-d₆) δ=7.21 (s, 1H), 4.42-4.53 (m, 2H), 3.55-3.64 (m, 2H), 3.06 (br s, 6H), 1.37-1.47 (m, 9H).

### Step 2—Tert-butyl 1-ethyl-4,6-dihydropyrrolo[3,4-c]pyrazole-5(1H)-carboxylate

To a solution of tert-butyl (3Z)-3-(dimethylaminomethylene)-4-oxo-pyrrolidine-1-carboxylate (500 mg, 2.08 mmol) and ethylhydrazine hydrochloride (332.14 mg, 2.50 mmol, HCl, CAS #18413-14-4) in EtOH (5 mL) was added TEA (421.10 mg, 4.16 mmol, 579.23 μL). The mixture was then stirred at 80° C. for 12 hrs. The mixture was diluted with ethyl acetate (15 mL) and water (15 mL). The mixture was extracted with ethyl acetate (3×15 mL). The combined organic extracts were washed with brine (10 mL) and then dried over anhydrous sodium sulfate, filtered and concentrated under vacuum to give the title compound (580 mg) as yellow oil. ¹H NMR (400 MHz, DMSO-d₆) δ=7.22 (d, J=11.2 Hz, 1H), 4.48 (d, J=1.6 Hz, 2H), 4.28 (d, J=1.6 Hz, 2H), 4.01-4.05 (m, 2H), 1.43 (s, 9H), 1.28-1.33 (m, 3 H).

### Step 3—1-Ethyl-1,4,5,6-tetrahydropyrrolo[3,4-c]pyrazole and 2-ethyl-2,4,5,6-tetrahydropyrrolo[3,4-c]pyrazole

To a solution of tert-butyl 1-ethyl-4,6-dihydropyrrolo[3,4-c]pyrazole-5-carboxylate (1.95 g, 8.22 mmol) in DCM (20 mL) was added HCl/dioxane (4 M, 20 mL) and the mixture was stirred at 20° C. for 2 h. The residue was purified by prep-HPLC (column: CD06-Waters Xbridge C18 150*40*10 um;mobile phase: [water(NH₃H2O)-ACN];gradient:0%-30% B over 12 min) give 1-ethyl-1,4,5,6-tetrahydropyrrolo[3,4-c]pyrazole (53 mg, 3% yield) as red oil (LC-MS (ESI+) m/z 138.0 (M+H)⁺) and 2-ethyl-2,4,5,6-tetrahydropyrrolo[3,4-c]pyrazole (96 mg, 8% yield) as red oil. LC-MS (ESI+) m/z 138.0 (M+H)⁺.

### 1-(3-(4-Chloro-2-(1-ethyl-1,4,5,6-tetrahydropyrrolo[3,4-c]pyrazole-5-carbonyl)-7-fluoro-1H-indol-6-yl)-5,6-dihydropyridin-1(2H)-yl)ethanone (Intermediate NJ)

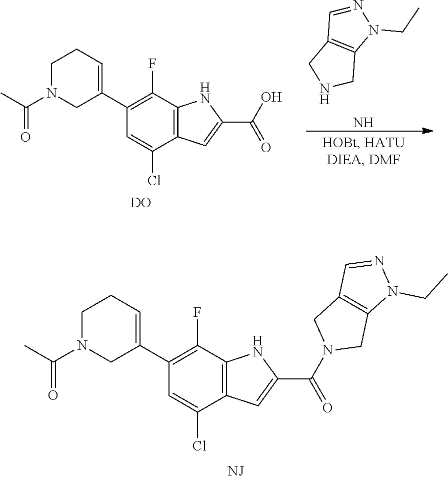

To a solution of 6-(1-acetyl-3,6-dihydro-2H-pyridin-5-yl)-4-chloro-7-fluoro-1H-indole-2-carboxylic acid (1 g, 2.97 mmol, Intermediate DO) and 1-ethyl-5,6-dihydro-4H-pyrrolo[3,4-c]pyrazole (515.65 mg, 2.97 mmol, HCl, Intermediate NH) in DMF (10 mL) was added HATU (1.35 g, 3.56 mmol), DIEA (1.15 g, 8.91 mmol, 1.55 mL) and HOBt (802.53 mg, 5.94 mmol). The mixture was then stirred at 25° C. for 0.5 hr. On completion, the mixture was diluted with ethyl acetate (10 mL) and water (10 mL). The mixture was extracted with ethyl acetate (3×10 mL). The combined organic extracts were washed with brine (10 mL) and then dried over anhydrous sodium sulfate, filtered and concentrated under vacuum. The residue was purified by column chromatography (SiO₂, DCM:MeOH=100/1 to 20/1) to give the title compound (700 mg, 43% yield) as a yellow solid. ¹H NMR (400 MHz, DMSO-d₆) δ 12.59 (br d, J=9.2 Hz, 1H), 7.36 (br d, J=10.4 Hz, 1H), 7.10-7.25 (m, 2H), 6.20 (br s, 1H), 5.21 (s, 1H), 5.01 (s, 1H), 4.88 (s, 1H), 4.68 (s, 1H), 4.35 (br s, 2H), 4.18 (m, 2H), 3.63-3.69 (m, 2H), 2.23-2.43 (m, 2H), 1.36-1.48 (m, 3H).

### 1-(3-(2-(1-Ethyl-1,4,5,6-tetrahydropyrrolo[3,4-c]pyrazole-5-carbonyl)-7-fluoro-4-(4,4,55-tetramethyl-1,3,2-dioxaborolan-2-yl)-1H-indol-6-yl)-5,6-dihydropyridin-1(2H)-yl)ethanone (Intermediate NK)

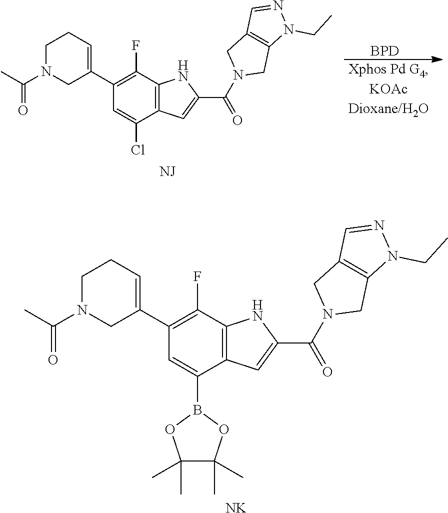

A mixture of 1-[5-[4-chloro-2-(1-ethyl-4,6-dihydropyrrolo[3,4-c]pyrazole-5-carbonyl)-7-fluoro-1H-indol-6-yl]-3,6-dihydro-2H-pyridin-1-yl]ethanone (350 mg, 767.69 μmol, Intermediate NJ), Xphos Pd G4 (66.06 mg, 76.77 μmol), BPD (389.89 mg, 1.54 mmol) and KOAc (226.03 mg, 2.30 mmol) in dioxane (5 mL) was degassed and purged with N₂ three times. Then the mixture was stirred at 90° C. for 12 hrs under N₂ atmosphere. On completion, the mixture was diluted with ethyl acetate (15 mL) and water (15 mL). The mixture was extracted with ethyl acetate (3×15 mL). The combined organic extracts were washed with brine (10 mL) and then dried over anhydrous sodium sulfate, filtered and concentrated under vacuum. The residue was purified by prep-TLC (SiO₂, DCM/MeOH=10/1) to give a 50% purity product. The crude product was purified by column chromatography (SiO₂, DCM:MeOH=100/1 to 10/1) to give the title compound (200 mg, 29% yield) as a yellow solid. ¹H NMR (400 MHz, CDCl₃-d) δ 9.59 (br d, J=9.2 Hz, 1H), 7.51-7.59 (m, 1H), 7.31-7.49 (m, 2H), 6.08-6.19 (m, 1H), 4.99-5.17 (m, 2H), 4.80-4.93 (m, 1H), 4.81 (s, 1H), 4.47 (br s, 1H), 4.32 (br s, 1H), 4.20 (m, 2H), 3.78 (m, 1H), 3.63 (m, 1H), 2.29-2.47 (m, 2H), 2.14-2.20 (m, 3H), 1.47-1.60 (m, 3H), 1.34-1.48 (m, 9H).

### 3-Bromo-4-methoxy-pyridine (CAS #82257-09-8) (Intermediate NL)

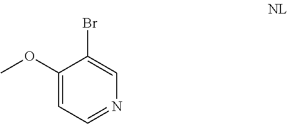

### 7-Bromo-2-(1-(tert-butoxycarbonyl)-1,2,5,6-tetrahydropyridin-3-yl)-1H-indole-5-carboxylic acid (Intermediate NM)

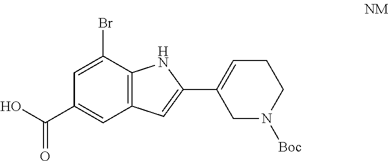 [SMILES: CC(C)(C)OC(=O)N1CCC=C(c2cc3cc(C(=O)O)cc(Br)c3[nH]2)C1]

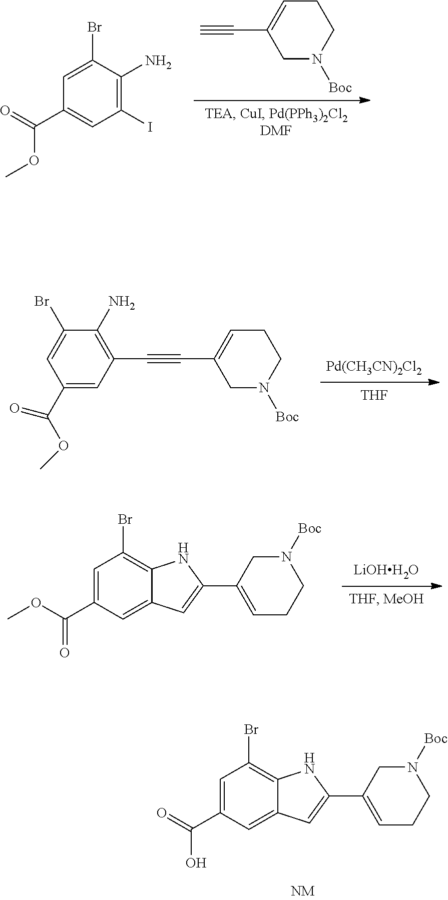

### Step 1—Tert-butyl 5-((2-amino-3-bromo-5-(methoxycarbonyl)phenyl)ethynyl)-3,6-dihydropyridine-1(2H)-carboxylate

A mixture of methyl 4-amino-3-bromo-5-iodobenzoate (8.98 g, 25.2 mmol, CAS #1240113-86-3), tert-butyl 5-ethynyl-3,6-dihydropyridine-1(2H)-carboxylate (6.8 g, 33 mmol, CAS #24424-99-5), Pd(PPh₃)₂Cl₂ (1.77 g, 2.52 mmol), CuI (576 mg, 3.03 mmol) and TEA (12.7 g, 126 mmol, 17.5 mL) in DMF (130 mL) was degassed and purged with N₂ three times. Then the mixture was stirred at 50° C. for 1 hr under N₂ atmosphere. On completion, the reaction mixture was triturated with H₂O at 0° C. for 30 min to give the title compound (10 g) as a white solid. LC-MS (ESI⁺) m/z 381.0 (M−56)⁺.

### Step 2—Methyl 7-bromo-2-(1-(tert-butoxycarbonyl)-1,2,5,6-tetrahydropyridin-3-yl′)-1H-indole-5-carboxylate

A mixture of tert-butyl 5-((2-amino-3-bromo-5-(methoxycarbonyl)phenyl)ethynyl)-3,6-dihydropyridine-1(2H)-carboxylate (5 g, 11 mmol), Pd(CH₃CN)Cl₂ (1 g, 6 mmol) in THF (50 mL) was degassed and purged with N₂ three times. Then the mixture was stirred at 80° C. for 0.5 hr under N₂ atmosphere. On completion, the reaction mixture was filtered and the filtrate was concentrated in vacuo to give the title compound (4.9 g) as a yellow oil. ¹H NMR (400 MHz, DMSO-d₆) δ=11.46 (s, 1H), 8.20 (s, 1H), 7.85 (d, J=1.2 Hz, 1H), 6.87-6.82 (m, 1H), 6.72 (br s, 1H), 3.85 (s, 3H), 3.79-3.75 (m, 2H), 3.42 (br s, 2H), 2.07 (s, 2H), 1.45 (s, 9H).

### Step 3—7-Bromo-2-(1-(tert-butoxycarbonyl)-1,2,5,6-tetrahydropyridin-3-yl)-1H-indole-5-carboxylic acid

To a solution of methyl 7-bromo-2-(1-(tert-butoxycarbonyl)-1,2,5,6-tetrahydropyridin-3-yl)-1H-indole-5-carboxylate (4.5 g, 10 mmol) in THF (40 mL), MeOH (10 mL) and H₂O (10 mL) was added LiOH·H₂O (1.08 g, 25.8 mmol). The mixture was then stirred at 25° C. for 12 hrs. On completion, the reaction mixture was quenched by addition of HCl (10 mL) at 0° C., and then diluted with H₂O (50 mL) and extracted with DCM (50 mL×3). The combined organic layers were dried over Na₂SO₄, filtered and concentrated under reduced pressure to give the title compound (4 g) as a white solid. LC-MS (ESI+) m/z 366.9 (M−55)⁺.

### Tert-butyl 3-(7-bromo-5-(4-(5-fluoro-3-methoxypyridin-2-yl)piperazine-1-carbonyl)-1H-indol-2-yl)-5,6-dihydropyridine-1(2H)-carboxylate (Intermediate NN)

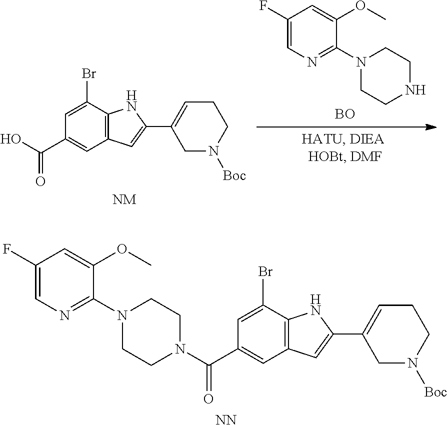

To a solution of 7-bromo-2-(1-(tert-butoxycarbonyl)-1,2,5,6-tetrahydropyridin-3-yl)-1H-indole-5-carboxylic acid (200 mg, 475 μmol, Intermediate NM) in DMF (2 mL) was added HOBt (70.6 mg, 522 μmol), DIEA (307 mg, 2.37 mmol), HATU (199 mg, 522 μmol) and 1-(5-fluoro-3-methoxypyridin-2-yl)piperazine (129 mg, 522 μmol, HCl, Intermediate BO). The mixture was stirred at 25° C. for 10 mins. On completion, water (4 ml) was added and a precipitate formed. Then the reaction mixture was filtered and the filter cake was dried in vacuo to give the title compound (250 mg) as a yellow solid. LC-MS (ESI⁺) m/z 614.2 (M+H)⁺.

### Tert-butyl 3-(5-(4-(5-fluoro-3-methoxypyridin-2-yl)piperazine-1-carbonyl)-7-(2-methoxyphenyl)-1H-indol-2-yl)-5,6-dihydropyridine-1(2H)-carboxylate (Intermediate NO)

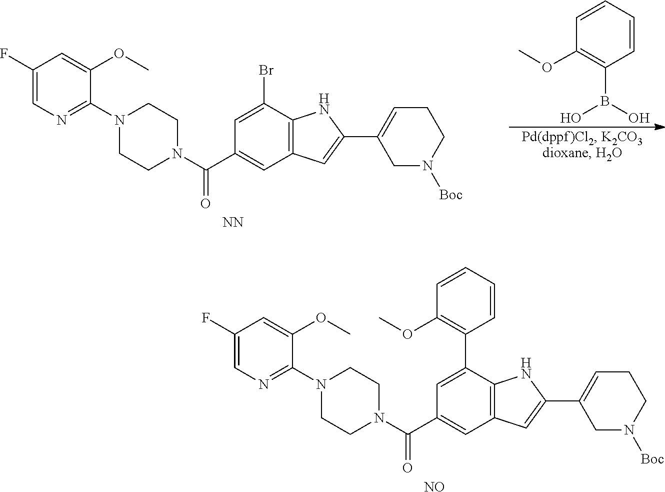

To a solution of tert-butyl 3-(7-bromo-5-(4-(5-fluoro-3-methoxypyridin-2-yl)piperazine-1-carbonyl)-1H-indol-2-yl)-5,6-dihydropyridine-1(2H)-carboxylate (250 mg, 407 μmol, Intermediate NN) and (2-methoxyphenyl)boronic acid (80.4 mg, 529 μmol, CAS #5720-06-9) in dioxane (2 mL) and H₂O (0.4 mL) was added Pd(dppf)Cl₂·CH₂Cl₂ (33.2 mg, 40.7 μmol) and K₂CO₃ (169 mg, 1.22 mmol). The mixture was stirred at 80° C. for 2 hrs. On completion, the reaction mixture was concentrated under reduced pressure to remove solvent, then the residue was purified by column chromatography (SiO₂, Petroleum ether/Ethyl acetate=1/1) to give the title compound (220 mg, 81% yield) as a yellow solid. LC-MS (ESI⁺) m/z 642.4 (M+H)⁺.

### (1—(Cyclopropylmethyl)-7-(2-methoxyphenyl)-2-(1,2,5,6-tetrahydropyridin-3-yl)-1H-indol-5-yl)(4-(5-fluoro-3-methoxypyridin-2-yl)piperazin-1-yl)methanone (Intermediate NP)

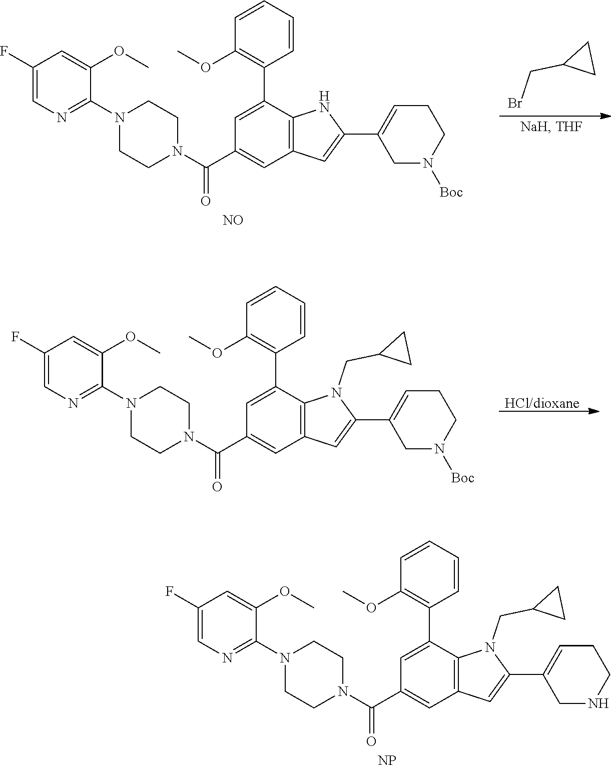

### Step 1—Tert-butyl 3-(1-(cyclopropylmethyl)-5-(4-(5-fluoro-3-methoxypyridin-2-yl)piperazine-1-carbonyl)-7-(2-methoxyphenyl)-1H-indol-2-yl)-5,6-dihydropyridine-1(2H)-carboxylate

To a solution of tert-butyl 3-(5-(4-(5-fluoro-3-methoxypyridin-2-yl)piperazine-1-carbonyl)-7-(2-methoxyphenyl)-1H-indol-2-yl)-5,6-dihydropyridine-1(2H)-carboxylate (220 mg, 343 μmol, Intermediate NO) in THF (3 mL) and was added NaH (20.6 mg, 514 μmol, 60% dispersion in mineral oil) at 0° C. under N₂ atmosphere. After 30 minutes of stirring, KI (5.69 mg, 34.3 μmol) and bromomethylcyclopropane (69.4 mg, 514 μmol, CAS #7051-34-5) was added, then the reaction was stirred at 25° C. for 3 hrs. On completion, the mixture was concentrated under reduced pressure to remove solvent. Then the mixture was purified by column chromatography (SiO₂, Petroleum ether/Ethyl acetate=1/1) to give the title compound (210 mg, 79% yield) as a yellow solid. LC-MS (ESI⁺) m/z 696.4 (M+H)⁺.

### Step 2—(1—(Cyclopropylmethyl)-7-(2-methoxyphenyl)-2-(1,2,5,6-tetrahydropyridin-3-yl)-1H-indol-5-yl)(4-(5-fluoro-3-methoxypyridin-2-yl)piperazin-1-yl)methanone

To a solution of tert-butyl 5-[1-(cyclopropylmethyl)-5-[4-(5-fluoro-3-methoxy-2-pyridyl)piperazine-1-carbonyl]-7-(2-methoxyphenyl)indol-2-yl]-3,6-dihydro-2H-pyridine-1-carboxylate (210 mg, 302 μmol) in DCM (3 mL) was added HCl/dioxane (2 M, 3 mL). The mixture was stirred at 25° C. for 1 hr. On completion, the mixture was concentrated under reduced pressure to give the title compound (300 mg, HCl) as a yellow solid. LC-MS (ESI⁺) m/z 596.6 (M+H)⁺.

### 7-Bromo-2-(1-tert-butoxycarbonyl-3,6-dihydro-2H-pyridin-5-yl)-1-(cyclopropylmethyl)indole-5-carboxylic acid (Intermediate NQ)

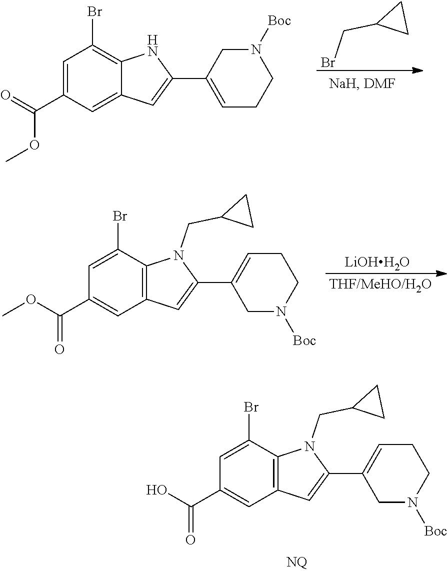

### Step 1—Methyl 7-bromo-2-(1-(tert-butoxycarbonyl)-1,2,5,6-tetrahydropyridin-3-yl)-1-(cyclopropylmethyl)-1H-indole-5-carboxylate

To a solution of methyl 7-bromo-2-(1-(tert-butoxycarbonyl)-1,2,5,6-tetrahydropyridin-3-yl)-1H-indole-5-carboxylate (3 g, 7 mmol, synthesized via Steps 1-2 of Intermediate NM) in DMF (30 mL) was added NaH (413 mg, 10.3 mmol, 60% dispersion in mineral oil) under N₂ at 0° C. for 0.5 hr. Then (bromomethyl)cyclopropane (1.12 g, 8.27 mmol, 789 μL, CAS #7051-34-5) was added at 0° C. The suspension was degassed under vacuum and purged with N₂ several times. The mixture was stirred under N₂ at 25° C. for 1.5 hrs. On completion, the reaction mixture was quenched by addition of NH₄Cl (30 mL) at 0° C., and extracted with DCM (30 mL×3). The combined organic layers were washed with aqueous NaCl (90 mL×3), dried over Na₂SO₄, filtered and concentrated under reduced pressure to give a residue. The residue was purified by prep-HPLC (neutral condition) to give the title compound (2 g, 27% yield) as a white solid. LC-MS (ESI+) m/z 491.0 (M+H)⁺.

### Step 2—7-Bromo-2-(1-(tert-butoxycarbonyl)-1,2,5,6-tetrahydropyridin-3-yl)-1-(cyclopropylmethyl)-1H-indole-5-carboxylic acid

To a solution of methyl 7-bromo-2-(1-(tert-butoxycarbonyl)-1,2,5,6-tetrahydropyridin-3-yl)-1-(cyclopropylmethyl)-1H-indole-5-carboxylate (2 g, 4 mmol) in THF (20 mL), MeOH (5 mL) and H₂O (5 mL) was added LiOH·H₂O (428 mg, 10.2 mmol). The mixture was stirred at 25° C. for 12 hrs. On completion, the reaction mixture was quenched by addition of HCl (5 mL) at 0° C., and then extracted with EA (20 mL×3). The combined organic layers were concentrated under reduced pressure to give a residue to give the title compound (1.8 g, HCl) as a white solid.

### (7-Bromo-1-(cyclopropylmethyl)-2-(1,2,5,6-tetrahydropyridin-3-yl)-1H-indol-5-yl)(1-methylpyrrolo[3,4-c]pyrazol-5(1H,4H,6H)-yl)methanone (Intermediate NR)

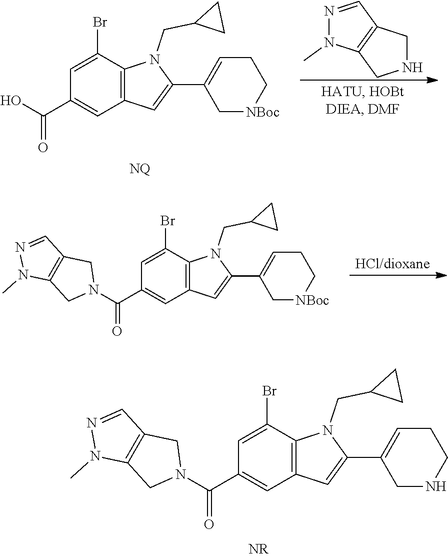

### Step 1—Tert-butyl 3-(7-bromo-1-(cyclopropylmethyl)-5-(1-methyl-1,4,5,6-tetrahydropyrrolo[3,4-c]pyrazole-5-carbonyl)-1H-indol-2-yl)-5,6-dihydropyridine-1(2H)-carboxylate

To a solution of 7-bromo-2-(1-tert-butoxycarbonyl-3,6-dihydro-2H-pyridin-5-yl)-1-(cyclopropylmethyl)indole-5-carboxylic acid (1.1 g, 2.3 mmol, Intermediate NQ) in DMF (10 mL) was added HATU (1.06 g, 2.78 mmol), HOBt (625 mg, 4.63 mmol) and DIEA (897 mg, 6.94 mmol). Then 1-methyl-5,6-dihydro-4H-pyrrolo[3,4-c]pyrazole hydrochloride (369 mg, 2.31 mmol, CAS #1187830-68-7) was added and the mixture was stirred at 25° C. for 10 mins. On completion, the reaction mixture was poured into H₂O (20 mL) at 0° C., and then filtered and the filter cake was dried under reduced pressure to give the title compound (1.4 g) as a brown gum. LC-MS (ESI⁺) m/z 580.3 (M+H)⁺; ¹H NMR (400 MHz, DMSO-d₆) δ=7.88-7.84 (m, 1H), 7.56 (d, J=8.8 Hz, 1H), 7.29-7.14 (m, 1H), 6.61 (s, 1H), 6.08 (s, 1H), 4.73 (d, J=8.0 Hz, 2H), 4.58 (d, J=7.6 Hz, 2H), 4.47 (d, J=6.4 Hz, 2H), 4.10 (s, 2H), 3.81-3.68 (m, 3H), 3.52 (t, J=5.2 Hz, 2H), 2.33-2.29 (m, 2H), 1.43 (s, 9H), 1.24 (s, 1H), 0.38 (d, J=6.4 Hz, 2H), 0.20 (s, 2H).

### Step 2—(7-Bromo-1-(cyclopropylmethyl)-2-(1,2,5,6-tetrahydropyridin-3-yl)-1H-indol-5-yl)(1-methylpyrrolo[3,4-c]pyrazol-5(1H,4H,6H)-yl)methanone

A solution of tert-butyl 5-[7-bromo-1-(cyclopropylmethyl)-5-(1-methyl-4,6-dihydropyrrolo[3,4-c]pyrazole-5-carbonyl)indol-2-yl]-3,6-dihydro-2H-pyridine-1-carboxylate (1.4 g, 2.41 mmol) in HCl/dioxane (2 M, 14 mL) was stirred at 25° C. for 1 hr. On completion, the reaction mixture was concentrated under reduced pressure to give a residue. The crude product was triturated with Petroleum ether:Ethyl acetate=10:1 at 25° C. for 30 mins to afford the title compound (1.25 g, 80% yield) as a brown solid. LC-MS (ESI⁺) m/z 480.2 (M+H)⁺; ¹H NMR (400 MHz, DMSO-d₆) δ=7.88 (d, J=7.6 Hz, 1H), 7.61-7.55 (m, 1H), 7.28-7.13 (m, 1H), 6.66 (s, 1H), 6.18 (s, 1H), 4.73 (d, J=8.4 Hz, 2H), 4.57 (d, J=10.0 Hz, 2H), 4.44 (d, J=6.4 Hz, 2H), 3.81-3.69 (m, 3H), 3.26 (d, J=5.2 Hz, 2H), 3.16 (s, 1H), 2.89 (s, 1H), 2.73-2.69 (m, 1H), 2.53 (s, 2H), 1.10 (t, J=5.6 Hz, 1H), 0.34 (d, J=7.6 Hz, 2H), 0.29 (d, J=3.6 Hz, 2H).

### 1-(3-(7-Bromo-1-(cyclopropylmethyl)-5-(1-methyl-1,4,5,6-tetrahydropyrrolo[3,4-c]pyrazole-5-carbonyl)-1H-indol-2-yl)-5,6-dihydropyridin-1(2H)-yl)-3-(1H-1,23-triazol-1-yl)propan-1-one (Intermediate NS)

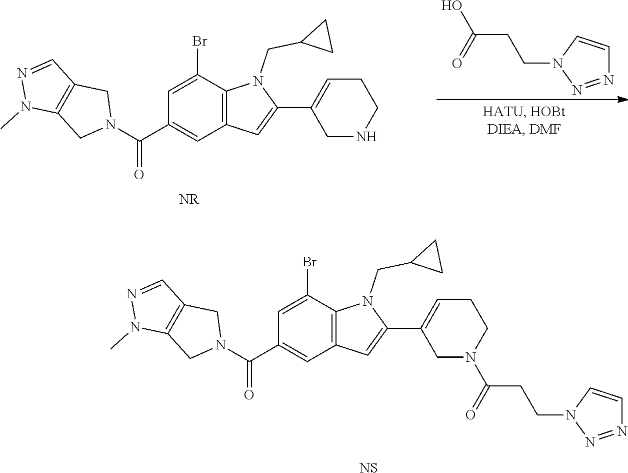 [SMILES: Cn1ncc2c1CN(C(=O)c1cc(Br)c3c(c1)cc(C1=CCCNC1)n3CC1CC1)C2 ; O=C(O)CCn1ccnn1 ; Cn1ncc2c1CN(C(=O)c1cc(Br)c3c(c1)cc(C1=CCCN(C(=O)CCn4ccnn4)C1)n3CC1CC1)C2 ; "NS"]

To a solution of 3-(triazol-1-yl)propanoic acid (352 mg, 2.50 mmol, CAS #4332-45-0) in DMF (12 mL) was added HATU (1.14 g, 3.00 mmol), DIEA (968 mg, 7.49 mmol) and HOBt (675 mg, 5.00 mmol). Then [7-bromo-1-(cyclopropylmethyl)-2-(1,2,3,6-tetrahydropyridin-5-yl)indol-5-yl]-(1-methyl-4,6-dihydropyrrolo[3,4-c]pyrazol-5-yl)methanone (1.2 g, 2.5 mmol, Intermediate NR) was added and the mixture was stirred at 25° C. for 10 mins. On completion, the reaction mixture was diluted with H₂O (30 mL) and extracted with EA (10 mL×3). The combined organic layers were washed with aqueous NaCl (10 mL×2), dried over Na₂SO₄, filtered and concentrated under reduced pressure to give a residue. The residue was purified by column chromatography (SiO₂, DCM/Ethyl acetate=30/1 to DCM:MeOH=6:1) to afford the title compound (800 mg, 47% yield) as a white solid. LC-MS (ESI⁺) m/z 603.2 (M+H)⁺; ¹H NMR (400 MHz, DMSO-d₆) δ=8.12 (d, J=3.6 Hz, 1H), 7.88-7.84 (m, 1H), 7.72-7.68 (m, 1H), 7.56 (d, J=8.4 Hz, 1H), 7.28-7.14 (m, 1H), 6.77-6.60 (m, 1H), 6.16-6.07 (m, 1H), 4.73 (d, J=8.4 Hz, 2H), 4.66-4.64 (m, 1H), 4.63-4.61 (m, 1H), 4.58 (s, 1H), 4.56 (s, 1H), 4.50-4.44 (m, 2H), 4.22 (d, J=7.2 Hz, 2H), 3.81-3.68 (m, 3H), 3.66-3.59 (m, 2H), 3.12 (t, J=6.8 Hz, 2H), 2.38-2.28 (m, 2H), 1.09-1.01 (m, 1H), 0.36 (t, J=6.8 Hz, 2H), 0.23-0.17 (m, 2H).

### Methyl 7-bromo-2-(piperidin-3-yl)-1H-indole-5-carboxylate (Intermediate NT)

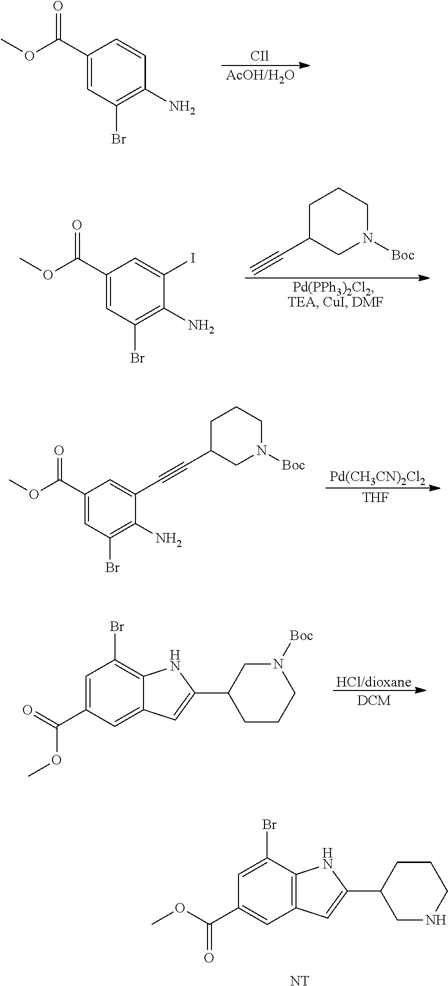

### Step 1—Methyl 4-amino-3-bromo-5-iodobenzoate

To a solution of methyl 4-amino-3-bromobenzoate (124 g, 539 mmol, CAS #106896-49-5) in AcOH (500 mL) and H₂O (500 mL) was added ClI (87.5 g, 539 mmol, 3.02 mL). The mixture was then stirred at 70° C. for 3 hrs. On completion, the reaction mixture was filtered and the filtrate cake was concentrated in vacuo to give the title compound (140 g) as a yellow solid. ¹H NMR (400 MHz, DMSO-d₆) δ=8.12 (d, J=2.0 Hz, 1H), 7.92 (d, J=2.0 Hz, 1H), 5.99 (s, 2H), 3.77 (s, 3H).

### Step 2—Tert-butyl 3-((2-amino-3-bromo-5-(methoxycarbonyl)phenyl)ethynyl)piperidine-1-carboxylate

A mixture of methyl 4-amino-3-bromo-5-iodobenzoate (30 g, 84.3 mmol), tert-butyl 3-ethynylpiperidine-1-carboxylate (18.5 g, 88.5 mmol, CAS #664362-16-7), Pd(PPh₃)₂Cl₂ (5.92 g, 8.43 mmol), CuI (1.61 g, 8.43 mmol) and TEA (42.6 g, 421 mmol, 58.7 mL) in DMF (500 mL) was degassed and purged with N₂ three times. Then the mixture was stirred at 50° C. for 2 hrs under N₂ atmosphere. On completion, the reaction mixture was diluted with water (200 mL) and extracted with ethyl acetate (3×100 mL). The combined organic layers were washed with brine (200 mL) and dried over anhydrous sodium sulfate, filtered and the filtrate was concentrated in vacuo to give the crude residue. The residue was purified by column chromatography (SiO₂, Petroleum ether:Ethyl acetate=10/1 to 4/1) to give the title compound (34 g, 75% yield) as a yellow oil. LC-MS (ESI+) m/z 337.0 (M−99)⁺.

### Step 3—Methyl 7-bromo-2-(1-(tert-butoxycarbonyl)piperidin-3-yl)-1H-indole-5-carboxylate

A mixture of tert-butyl 3-((2-amino-3-bromo-5-(methoxycarbonyl)phenyl)ethynyl)piperidine-1-carboxylate (30 g, 54 mmol) and Pd(CH₃CN)₂Cl₂ (6.94 g, 26.8 mmol) in THF (300 mL) was degassed and purged with N₂ three times. Then the mixture was stirred at 80° C. for 0.5 hrs under N₂ atmosphere. On completion, the reaction mixture was concentrated in vacuo to give the crude residue. The residue was purified by column chromatography (SiO₂, Petroleum ether: Ethyl acetate=1/0 to 4/1) to give a title compound (16 g, 67% yield) as a white solid. LC-MS (ESI+) m/z 437.0 (M+H)⁺; ¹H NMR (400 MHz, DMSO-d₆) δ=11.61 (br d, J=2.8 Hz, 1H), 8.15 (d, J=1.2 Hz, 1H), 7.80 (d, J=1.2 Hz, 1H), 6.52 (s, 1H), 4.10 (br d, J=4.4 Hz, 3H), 3.84 (s, 3H), 2.92 (br d, J=11.6 Hz, 2H), 2.07 (br d, J=9.6 Hz, 1H), 1.72 (br d, J=10.8 Hz, 2H), 1.46 (br s, 1H), 1.38 (br s, 9H).

### Step 4—Methyl 7-bromo-2-(piperidin-3-yl)-1H-indole-5-carboxylate

To a solution of methyl 7-bromo-2-(1-(tert-butoxycarbonyl)piperidin-3-yl)-1H-indole-5-carboxylate (6.8 g, 15.5 mmol) in DCM (50 mL) was added HCl/dioxane (2 M, 100 mL). The mixture was then stirred at 25° C. for 1 hr. On completion, the reaction mixture was filtered and the filtrate cake was concentrated in vacuo to give the title compound (6.5 g) as a pink solid. LC-MS (ESI+) m/z 337.0 (M+H)⁺; ¹H NMR (400 MHz, DMSO-d₆) δ=11.74 (s, 1H), 9.38 (br s, 2H), 8.18 (d, J=0.8 Hz, 1H), 7.82 (d, J=1.2 Hz, 1H), 6.56 (d, J=1.6 Hz, 1H), 3.84 (s, 3H), 3.56 (s, 4H), 3.20-3.06 (m, 1H), 2.86 (br s, 1H), 2.14 (br d, J=11.6 Hz, 1H), 1.94-1.82 (m, 2H).

### Methyl (S)-2-(1-(3-(1H-1,2,3-triazol-1-yl)propanoyl)piperidin-3-yl)-7-bromo-1H-indole-5-carboxylate (Intermediate NU) and methyl (R)-2-(1-(3-(1H-1,2,3-triazol-1-yl)propanoyl)piperidin-3-yl)-7-bromo-1H-indole-5-carboxylate (Intermediate NV)

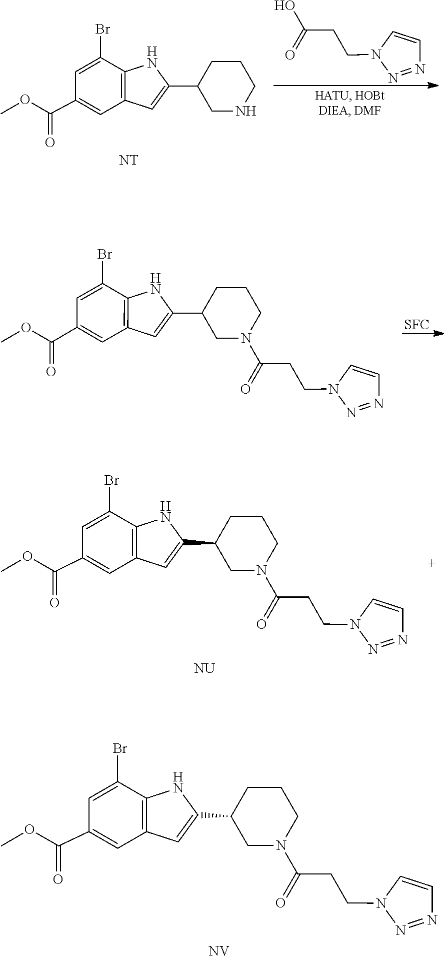

### Step 1—Methyl 2-(1-(3-(1H-1,2,3-triazol-1-yl)propanoyl)piperidin-3-yl)-7-bromo-1H-indole-5-carboxylate

To a solution of 3-(1H-1,2,3-triazol-1-yl)propanoic acid (2.3 g, 16.3 mmol) in DMF (100 mL) was added HATU (7.44 g, 19.6 mmol), HOBt (4.40 g, 32.6 mmol), DIEA (10.5 g, 81.5 mmol) and methyl 7-bromo-2-(piperidin-3-yl)-1H-indole-5-carboxylate (6.09 g, 16.3 mmol, Intermediate NT). The mixture was then stirred at 25° C. for 10 min. On completion, the reaction mixture was diluted with water (200 mL) and filtered. The filter cake was concentrated in vacuo to give the crude residue. The residue was purified by column chromatography (SiO₂, Petroleum ether:Ethyl acetate=1/1) (SiO₂, Dichloromethane: Methanol=20/1) to give the title compound (6 g, 79% yield) as a yellow solid. LC-MS (ESI+) m/z 462.1 (M+H)⁺; ¹H NMR (400 MHz, DMSO-d₆) δ=11.64 (s, 1H), 8.16 (d, J=4.0 Hz, 1H), 8.10 (d, J=6.0 Hz, 1H), 7.81 (dd, J=1.2, 3.2 Hz, 1H), 7.70 (s, 1H), 6.53 (dd, J=1.6, 11.2 Hz, 1H), 4.62 (brt, J=6.4 Hz, 2H), 3.84 (s, 3H), 3.20 (td, J=7.2, 16.8 Hz, 1H), 3.09-3.02 (m, 2H), 2.90-2.84 (m, 2H), 2.73 (s, 1H), 2.62 (br t, J=11.6 Hz, 1H), 2.15-2.08 (m, 1H), 1.80-1.72 (m, 2H), 1.50-1.38 (m, 1H).

### Step 2—Methyl (S)-2-(1-(3-(1H-1,2,3-triazol-1-yl)propanoyl)piperidin-3-yl)-7-bromo-1H-indole-5-carboxylate and methyl (R)-2-(1-(3-(1H-1,2,3-triazol-1-yl)propanoyl)piperidin-3-yl)-7-bromo-1H-indole-5-carboxylate

Methyl 2-(1-(3-(1H-1,2,3-triazol-1-yl)propanoyl)piperidin-3-yl)-7-bromo-1H-indole-5-carboxylate was separated by SFC (column: DAICEL CHIRALPAK IG (250 mm*30 mm,10 um); mobile phase: [CO₂-ACN/MeOH(0.1% NH₃H2O)]; B %:55%, isocratic elution mode) to give the first eluting isomer (S)-2-(1-(3-(1H-1,2,3-triazol-1-yl)propanoyl)piperidin-3-yl)-7-bromo-1H-indole-5-carboxylic acid (2.5 g, 51% yield) as a white solid (LC-MS (ESI+) m/z 462.1 (M+H)⁺) and the second eluting isomer methyl (R)-2-(1-(3-(1H-1,2,3-triazol-1-yl)propanoyl)piperidin-3-yl)-7-bromo-1H-indole-5-carboxylate (3 g, 61% yield). LC-MS (ESI+) m/z 462.1 (M+H)⁺ for both isomers. The absolute configuration of the enantiomers was assigned arbitrarily.

### (S)-2-(1-(3-(1H-1,2,3-triazol-1-yl)propanoyl)piperidin-3-yl)-7-bromo-1H-indole-5-carboxylic acid (Intermediate NW)

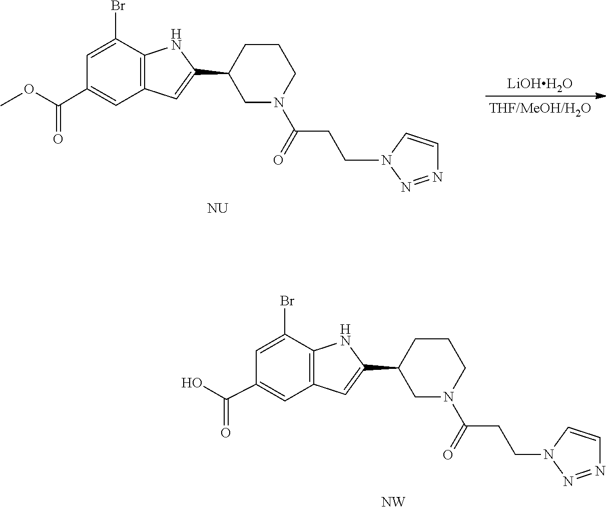 [SMILES: COC(=O)c1cc(Br)c2[nH]c([C@H]3CCCN(C(=O)CCn4ccnn4)C3)cc2c1 ; "LiOH·H2O" ; "NU" ; O=C(O)c1cc(Br)c2[nH]c([C@H]3CCCN(C(=O)CCn4ccnn4)C3)cc2c1 ; "NW"]

To a solution of (S)-methyl 2-(1-(3-(1H-1,2,3-triazol-1-yl)propanoyl)piperidin-3-yl)-7-bromo-1H-indole-5-carboxylate (2.4 g, 5.2 mmol, Intermediate NU) in THF (16 mL), MeOH (4 mL) and H₂O (4 mL) was added LiOH·H₂O (656 mg, 15.6 mmol). The mixture was then stirred at 25° C. for 12 hrs. On completion, HCl was added to the mixture until pH was 5.0 and extracted with ethyl acetate (3×50 mL). The combined organic layers were concentrated in vacuo to give the title compound (2 g) as a yellow solid. LC-MS (ESI+) m/z 448.0 (M+H)⁺.

### Tert-butyl 3-(7-bromo-5-(dimethylcarbamoyl)-3-fluoro-1H-indol-2-yl)piperidine-1-carboxylate (Intermediate NX)

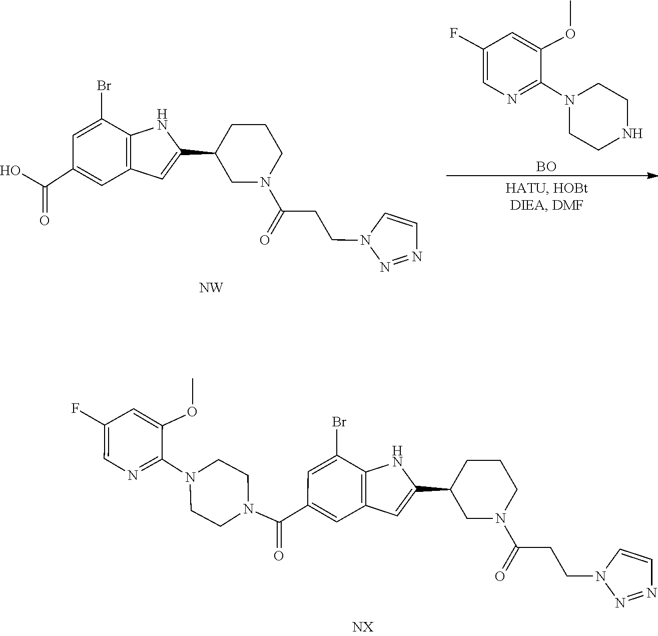 [SMILES: O=C(O)c1cc(Br)c2[nH]c([C@H]3CCCN(C(=O)CCn4ccnn4)C3)cc2c1 ; COc1cc(F)cnc1N1CCNCC1 ; "BO" ; "NW" ; COc1cc(F)cnc1N1CCN(C(=O)c2cc(Br)c3[nH]c([C@H]4CCCN(C(=O)CCn5ccnn5)C4)cc3c2)CC1]

To a solution of (S)-2-(1-(3-(1H-1,2,3-triazol-1-yl)propanoyl)piperidin-3-yl)-7-bromo-1H-indole-5-carboxylic acid (500 mg, 1.12 mmol, Intermediate NW) in DMF (10 mL) was added HATU (511 mg, 1.34 mmol), HOBt (303 mg, 2.24 mmol), DIEA (724 mg, 5.60 mmol) and 1-(5-fluoro-3-methoxypyridin-2-yl)piperazine (284 mg, 1.34 mmol, Intermediate BO). The mixture was stirred at 25° C. for 10 min. On completion, the reaction mixture was quenched with water (20 mL) and filtered. The filter cake was dried in vacuo to give the title compound (1 g) as a yellow solid. LC-MS (ESI+) m/z 639.2 (M+H)⁺;

### (S)-1-(3-(7-bromo-5-(1-methyl-1,4,5,6-tetrahydropyrrolo[3,4-c]pyrazole-5-carbonyl)-1H-indol-2-yl)piperidin-1-yl)-3-(1H-1,2,3-triazol-1-yl)propan-1-one (Intermediate NY)

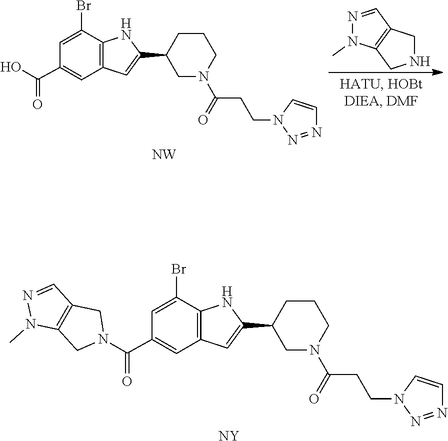

To a solution of (S)-2-(1-(3-(1H-1,2,3-triazol-1-yl)propanoyl)piperidin-3-yl)-7-bromo-1H-indole-5-carboxylic acid (500 mg, 1.12 mmol, Intermediate NW) in DMF (6 mL) was added HOBt (181 mg, 1.34 mmol), HATU (553 mg, 1.46 mmol), DIEA (579 mg, 4.48 mmol, 780 μL) and 1-methyl-1,4,5,6-tetrahydropyrrolo[3,4-c]pyrazole (165 mg, 1.34 mmol, CAS #762233-62-5), then the reaction was stirred at 25° C. for 10 min On completion, the reaction mixture was diluted with water (100 mL) and filtered. The filter cake was concentrated in vacuo to give the title compound (500 mg) as a yellow solid. LC-MS (ESI⁺) m/z 551.1 (M+H)⁺.

### 2-[(4-Methoxy-5-piperazin-1-yl-pyrazol-1-yl)methoxy]ethyl-trimethyl-silane (Intermediate NZ)

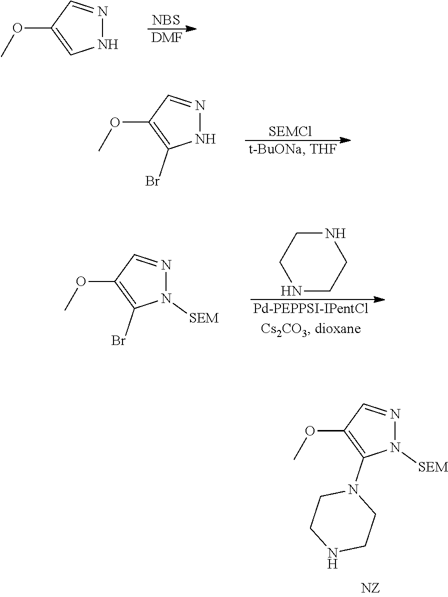

### Step 1—5-Bromo-4-methoxy-1H-pyrazole

To a solution of 4-methoxy-1H-pyrazole (5.00 g, 51.0 mmol, CAS #14884-01-6) in DMF (75 mL) was added NBS (9.07 g, 51.0 mmol). The mixture was then stirred at 20° C. for 14 h. On completion, the mixture was concentrated in vacuo. The residue was purified by column chromatography (PE/EtOAc=10/1 to 3/1) to give the title compound (6.60 g, 70% yield) as a white solid. LC-MS (ESI⁺) m/z 176.9/178.9 (M+H)⁺; ¹H NMR (400 MHz, DMSO-d₆) δ 12.71 (s, 1H), 7.56 (s, 1H), 3.70 (s, 3H).

### Step 2—2-[(5-Bromo-4-methoxy-pyrazol-1-yl)methoxy]ethyl-trimethyl-silane

To a solution 5-bromo-4-methoxy-1H-pyrazole (600.00 mg, 3.39 mmol) in THF (15 mL) at 0° C. was added t-BuONa (425 mg, 4.42 mmol). The mixture was stirred at 0° C. for 30 min, and the reaction mixture was treated with SEM-Cl (659.40 mg, 3.96 mmol) and was then stirred for 2 h. On completion, the mixture was concentrated in vacuo. The residue was purified by column chromatography (PE/EtOAc=1/0 to 10/1) to give the title compound (1.00 g, 91% yield) as a colorless oil. LC-MS (ESI⁺) m/z 248.9/250.9 (M+H)⁺.

### Step 3—2-[(4-Methoxy-5-piperazin-1-yl-pyrazol-1-yl)methoxy]ethyl-trimethyl-silane

To a solution of 2-[(5-bromo-4-methoxy-pyrazol-1-yl)methoxy]ethyl-trimethyl-silane (500.00 mg, 1.63 mmol) and piperazine (280.33 mg, 3.25 mmol) in dioxane (10 mL) was added 1,3-bis[2,6-bis(1-ethylpropyl)phenyl]-4,5-dichloro-2H-imidazol-1-ium-2-ide;3-chloropyridine dichloropalladium (140.04 mg, 162.73 μmol) and Cs₂CO₃ (1.59 g, 4.88 mmol). The mixture was then stirred at 80° C. for 2 h. On completion, the mixture was diluted with H₂O (3 mL) and extracted with EtOAc (3 mL×3). The combined organic layers were washed with NaCl (3 mL×3), dried over Na₂SO₄, filtered and concentrated under reduced pressure to give a residue. The residue was purified by column chromatography (DCM/MeOH=10/1 to 5/1) to give the title compound (130.00 mg, 24% yield) as a yellow oil. LC-MS (ESI⁺) m/z 313.2 (M+H)⁺; ¹H NMR (400 MHz, CDCl₃-d) δ 7.08 (s, 1H), 5.17 (s, 2H), 3.74 (s, 3H), 3.55-3.47 (m, 2H), 3.27-3.19 (m, 4H), 3.07-3.00 (m, 4H), 0.96-0.79 (m, 3H), −0.02 (s, 9H).

### 5-Bromo-4-methoxy-2, 3-dimethyl-pyridine (Intermediate OA)

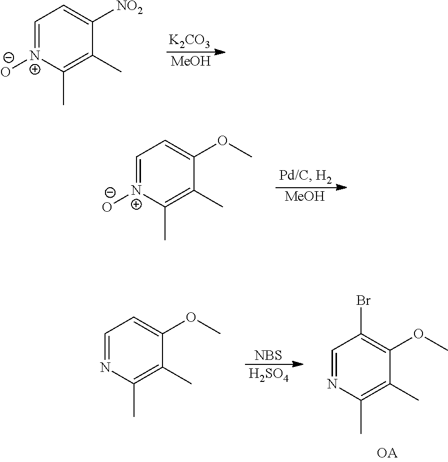

### Step 1—4-Methoxy-2, 3-dimethylpyridine 1-oxide

To a solution of 2, 3-dimethyl-4-nitro-1-oxido-pyridin-1-ium (2.00 g, 11.9 mmol, CAS #37699-43-7) in MeOH (20 mL) was added K₂CO₃ (3.29 g, 23.8 mmol). The mixture was stirred at 40° C. for 2 h. The reaction mixture was diluted with H₂O (10 mL) and extracted with EtOAc (10 mL×3). The combined organic layers were washed with NaCl (10 mL×3), dried over Na₂SO₄, filtered and concentrated under reduced pressure to give a residue. The residue was purified by column chromatography (DCM/MeOH=10/1 to 9/1) to give the title compound (1.00 g, 55% yield) as a yellow solid. LC-MS (ESI⁺) m/z 154.2 (M+H)⁺.

### Step 2—4-Methoxy-2, 3-dimethyl-pyridine

To a solution of 4-methoxy-2, 3-dimethyl-1-oxido-pyridin-1-ium (1.00 g, 6.53 mmol) in MeOH (20 mL) was added Pd/C (347.37 mg, 326.42 μmol, 10 wt %) under H₂. The mixture was stirred at 20° C. for 12 h. The reaction mixture was filtered and concentrated under reduced pressure to give the title compound (800 mg, 89% yield) as a gray solid. LC-MS (ESI⁺) m/z 138.2 (M+H)⁺; ¹H NMR (400 MHz, DMSO-d₆) δ 8.14 (d, J=5.6 Hz, 1H), 6.83 (d, J=5.6 Hz, 1H), 3.81 (s, 3H), 2.37 (s, 3H), 2.06 (s, 3H).

### Step 3—5-Bromo-4-methoxy-2, 3-dimethyl-pyridine

To a solution of 4-methoxy-2, 3-dimethyl-pyridine (100.00 mg, 728.98 μmol) in H₂SO₄ (1 mL) was added NBS (142.72 mg, 801.87 μmol) at 60° C. The mixture was stirred at 60° C. for 3 h. The reaction mixture was quenched by of addition H₂O (5 mL) at 0° C., basified with K₂CO₃ and extracted with DCM (5 mL×3). The combined organic layers were washed with NaCl (5 mL×3), dried over Na₂SO₄, filtered and concentrated under reduced pressure to give the title compound (100.00 mg, 64% yield) as a yellow oil. LC-MS (ESI⁺) m/z 216.0/218.0 (M+H)⁺.

### 2-[(4-Ethyl-5-piperazin-1-yl-pyrazol-1-yl)methoxy]ethyl-trimethyl-silane (Intermediate OB)

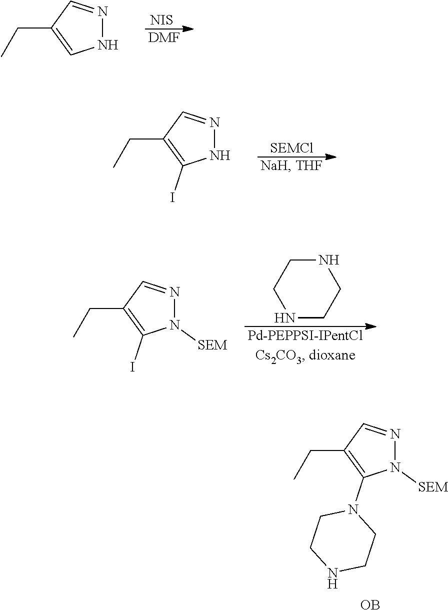

### Step 1—4-Ethyl-5-iodo-1H-pyrazole

To a solution of 4-ethyl-1H-pyrazole (800 mg, 8.32 mmol, CAS #17072-38-7) in ACN (10 mL) was added NIS (1.87 g, 8.32 mmol). The mixture was then stirred at 60° C. for 0.5 hr. On completion, the mixture was diluted with H₂O (20 mL) and extracted with EtOAc (10 mL×2). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 0˜50% EtOAc/PE gradient @80 mL/min) to give the title compound (1 g, 54% yield) as a yellow gum. LC-MS (ESI⁺) m/z 222.9 (M+1); ¹H NMR (400 MHz, DMSO-d₆) δ=12.98 (br s, 1H), 7.50 (br s, 1H), 2.29 (q, J=7.6 Hz, 2H), 1.10 (t, J=7.6 Hz, 3H).

### Step 2—2-[(4-Ethyl-5-iodo-pyrazol-1-yl)methoxy]ethyl-trimethyl-silane

To a solution of 4-ethyl-5-iodo-1H-pyrazole (1 g, 5 mmol) in THF (10 mL) was added NaH (360 mg, 9.01 mmol, 60% dispersion in mineral oil) at 20° C. under N₂ atmosphere and stirred at 20° C. for 0.5 h. Then SEM-Cl (751 mg, 4.50 mmol, 797.14 μL) was added to the mixture dropwise and stirred at 20° C. for 2 h. On completion, the mixture was diluted with saturated solution of NH₄Cl (30 mL) and extracted with EtOAc (20 mL×2). The combined organic layers were dried over Na₂SO₄, filtered and concentrated under reduced pressure to give the title compound (1.6 g) as a yellow gum. LC-MS (ESI⁺) m/z 353.3 (M+1)⁺.

### Step 3—2-[(4-Ethyl-5-piperazin-1-yl-pyrazol-1-yl)methoxy]ethyl-trimethyl-silane

A mixture of 2-[(4-ethyl-5-iodo-pyrazol-1-yl)methoxy]ethyl-trimethyl-silane (500 mg, 1.42 mmol), piperazine (611 mg, 7.10 mmol), Pd-PEPPSI-IPentCl (122 mg, 141.93 μmol) and Cs₂CO₃ (925 mg, 2.84 mmol) in dioxane (5 mL) was degassed and purged with N₂ three times. Then the mixture was stirred at 100° C. for 2 hr under N₂ atmosphere. On completion, the mixture was filtered and the filtrate was concentrated under reduced pressure to give a residue. The residue was purified by prep-HPLC (FA condition) to give the title compound (120 mg, 25% yield) as a yellow gum. LC-MS (ESI⁺) m/z 311.2 (M+1)⁺; ¹H NMR (400 MHz, DMSO-d₆) δ=7.65 (s, 1H), 5.31 (s, 2H), 3.50 (t, J=8.0 Hz, 2H), 3.32 (s, 8H), 2.29 (q, J=7.2 Hz, 2H), 1.14-1.09 (m, 3H), 0.81 (t, J=8.0 Hz, 2H), −0.03-−0.07 (m, 9H).

### 1-(4,5-Difluoro-3-methoxypyridin-2-yl)piperazine (Intermediate OC)

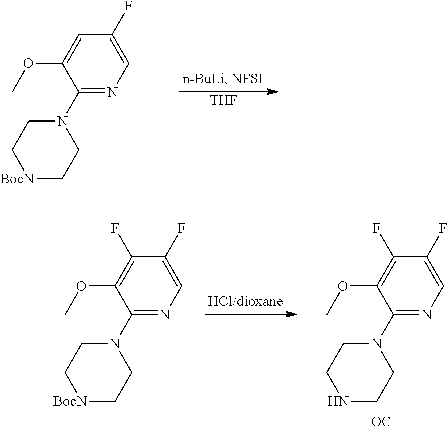

### Tert-butyl 4-(4,5-difluoro-3-methoxypyridin-2-yl)piperazine-1-carboxylate

To a solution of tert-butyl 4-(5-fluoro-3-methoxy-2-pyridyl)piperazine-1-carboxylate (0.1 g, 320 μmol) in THF (15 mL) was added n-BuLi (2.5 M, 282.64 μL) at −70° C. and the mixture was stirred for 1 h under N₂ atmosphere. Then NFSI (202.56 mg, 642.36 μmol) was added dropwise (in THF 5 mL), and resulting mixture was stirred for 1 h at −70° C. under nitrogen atmosphere. On completion, the reaction mixture was quenched by of addition water (30 mL), and then diluted with ethyl acetate (10 mL). The aqueous phase was extracted with ethyl acetate (50 mL). The combined organic phase was washed with brine (50 mL), dried with anhydrous Na₂SO₄, filtered and concentrated in vacuo to give a residue. The residue was purified by prep-TLC (PE/EA=3/1) to give the title compound (100 mg, 95% yield) as a yellow oil. LC-MS (ESI⁺) m/z 330.0 (M+H)⁺; ¹H NMR (400 MHz, CDCl₃): δ=7.83 (d, J=8.4 Hz, 1H), 7.19 (s, 2H), 3.86 (d, J=1.2 Hz, 3H), 3.51-3.43 (m, 4H), 3.31-3.21 (m, 4H), 1.41 (s, 9H).

### Step 2—1-(4,5-Difluoro-3-methoxypyridin-2-yl)piperazine

To a solution of tert-butyl 4-(4,5-difluoro-3-methoxy-2-pyridyl)piperazine-1-carboxylate (100 mg, 303.64 μmol) in DCM (2 mL) was added TFA (1.54 g, 13.46 mmol). The mixture was then stirred at 20° C. for 1 h. On completion, the reaction mixture was concentrated in vacuum to give the title compound (100 mg, TFA) as a yellow oil. LC-MS (ESI⁺) m/z 230.0 (M+H)⁺.

### 4-(1-(tert-butoxycarbonyl)piperidin-4-yl)-6-chloro-7-fluoro-1H-indole-2-carboxylic acid (Intermediate OD)

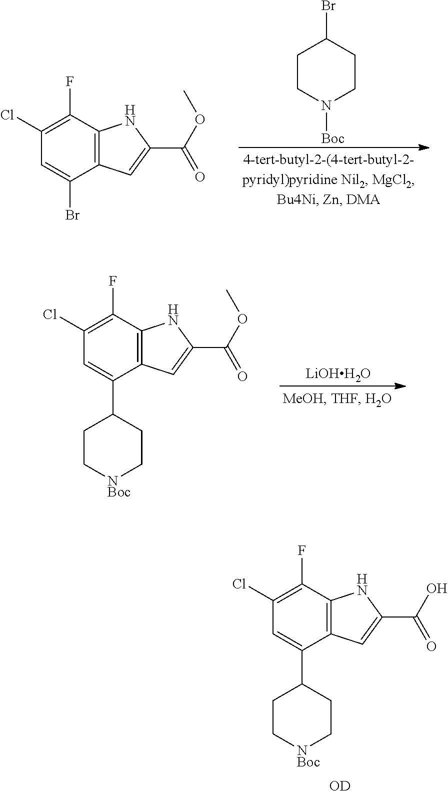

### Step 1—Methyl 4-(1-(tert-butoxycarbonyl)piperidin-4-yl)-6-chloro-7-fluoro-1H-indole-2-carboxylate

To a solution of methyl 4-bromo-6-chloro-7-fluoro-1H-indole-2-carboxylate (1.50 g, 4.89 mmol, synthesized via Steps 1-5 of Intermediate C) in DMA (30.0 mL) was added tert-butyl 4-bromopiperidine-1-carboxylate (1.94 g, 7.34 mmol, CAS #180695-79-8), diiodonickel (152 mg, 489 μmol, 26.2 μL), dichloromagnesium (466 mg, 4.89 mmol, 200.83 μL), tetrabutylammonium iodide (1.81 g, 4.89 mmol), 4-tert-butyl-2-(4-tert-butyl-2-pyridyl)pyridine (131 mg, 489 μmol), and Zinc (640 mg, 9.79 mmol). The reaction was then stirred at 60° C. for 5 hrs. On completion, the mixture was filtered and concentrated under reduced pressure to give a residue. The mixture was extracted with EA (50 mL×2) and was concentrated under reduced pressure to give a residue. The crude product was purified by reversed-phase HPLC (0.08% NH₄HCO₃ condition) to give the title compound (930 mg, 30% yield) as a yellow solid. ¹H NMR (400 MHz, DMSO-d₆) δ=12.70 (s, 1H), 7.42 (d, J=2.8 Hz, 1H), 6.98 (d, J=6.0 Hz, 1H), 3.88 (s, 3H), 3.18 (t, J=12.0 Hz, 1H), 2.92 (dd, J=2.4, 5.6 Hz, 2H), 1.80 (d, J=12.4 Hz, 2H), 1.61 (d, J=12.8 Hz, 4H), 1.38 (s, 9H).

### Step 2—4-(1-(Tert-butoxycarbonyl)piperidin-4-yl)-6-chloro-7-fluoro-1H-indole-2-carboxylic acid

To a solution of methyl 4-(1-tert-butoxycarbonyl-4-piperidyl)-6-chloro-7-fluoro-1H-indole-2-carboxylate (930 mg, 2.26 mmol) in THF (3.00 mL), MeOH (3.00 mL) and H₂O (3.00 mL) was added LiOH·H₂O (475 mg, 11.3 mmol). Then the mixture was stirred at 25° C. for 3 hrs. On completion, to the mixture was added HCl (2M) 10.0 mL, then the mixture was concentrated under reduced pressure to give the title compound (1.00 g) as a white solid. LC-MS (ESI⁺) m/z 395.0 (M−H)⁺.

### (6-Chloro-7-fluoro-4-(1-methylpiperidin-4-yl)-1H-indol-2-yl)(4-(5-fluoro-3-methoxypyridin-2-yl)piperazin-1-yl)methanone (Intermediate OE)

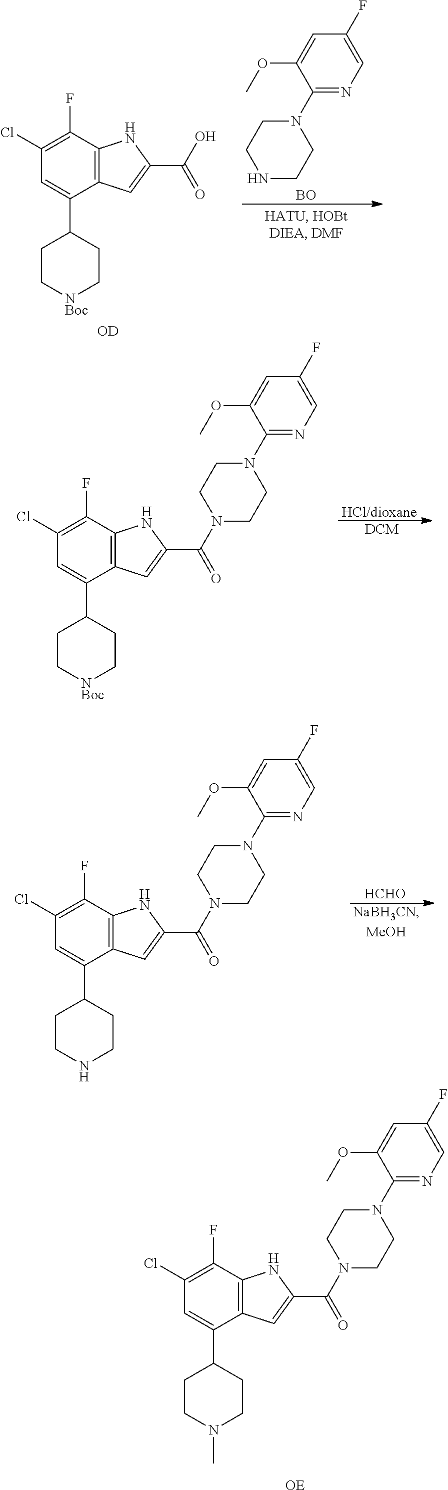

### Step 1—Tert-butyl 4-(6-chloro-7-fluoro-2-(4-(5-fluoro-3-methoxypyridin-2-yl)piperazine-1-carbonyl)-1H-indol-4-yl)piperidine-1-carboxylate

To a solution of 4-(1-tert-butoxycarbonyl-4-piperidyl)-6-chloro-7-fluoro-1H-indole-2-carboxylic acid (970 mg, 2.44 mmol, Intermediate OD) in DMF (10.0 mL) and was added HATU (929 mg, 2.44 mmol), HOBt (550 mg, 4.07 mmol) and DIEA (1.32 g, 10.2 mmol, 1.77 mL), then the mixture was stirred at 25° C. for 10 mins. Then 1-(5-fluoro-3-methoxy-2-pyridyl)piperazine (430 mg, 2.04 mmol, Intermediate BO) was added and the mixture was stirred at 25° C. for 0.5 hr. On completion, the mixture was dripped into H₂O, and then filtered and washed with H₂O and PE, then concentrated under reduced pressure to give the title compound (900 mg) as a yellow solid. ¹H NMR (400 MHz, CDCl₃-d) δ=9.52 (s, 1H), 6.95-6.89 (m, 2H), 6.81 (d, J=2.4 Hz, 1H), 4.34-4.26 (m, 2H), 4.15-4.03 (m, 6H), 3.92 (s, 3H), 3.48-3.42 (m, 4H), 3.04-2.98 (m, 1H), 1.91 (d, J=12.8 Hz, 2H), 1.72 (dd, J=4.0, 12.7 Hz, 2H), 1.46 (s, 9H).

### Step 2—(6-Chloro-7-fluoro-4-(piperidin-4-yl)-1H-indol-2-yl)(4-(5-fluoro-3-methoxypyridin-2-yl)piperazin-1-yl)methanone

To a solution of tert-butyl 4-[6-chloro-7-fluoro-2-[4-(5-fluoro-3-methoxy-2-pyridyl)piperazine-1-carbonyl]-1H-indol-4-yl]piperidine-1-carboxylate (900 mg, 1.53 mmol) in DCM (4.00 mL) was added HCl/dioxane (7 M, 4.00 mL), then the mixture was stirred at 25° C. for 1 hr. On completion, the mixture was concentrated under reduced pressure. The crude product was triturated with ACN and PE at 25° C. for 12 hrs, then filtered to give the title compound (540 mg, 46% yield) as a yellow solid. LC-MS (ESI⁺) m/z 490.2 (M+H)⁺.

### Step 3—(6-Chloro-7-fluoro-4-(1-methylpiperidin-4-yl)-1H-indol-2-yl)(4-(5-fluoro-3-methoxypyridin-2-yl)piperazin-1-yl)methanone

To a solution of [6-chloro-7-fluoro-4-(4-piperidyl)-1H-indol-2-yl]-[4-(5-fluoro-3-methoxy-2-pyridyl)piperazin-1-yl]methanone (240 mg, 490 μmol) in MeOH (2.00 mL) was added HCHO (398 mg, 4.90 mmol, 364.70 μL, 37% solution) and NaBH₃CN (92.4 mg, 1.47 mmol) at 0° C. The reaction was then stirred at 25° C. for 4 hrs. On completion, the mixture was dripped in H₂O, and then filtered and washed with H₂O and PE, and dried under reduced pressure to give the title compound (150 mg, 61% yield) as a yellow solid. ¹H NMR (400 MHz, DMSO-d₆) δ=12.45-12.21 (m, 1H), 7.85-7.72 (m, 1H), 7.39 (d, J=9.2 Hz, 1H), 7.06 (s, 1H), 6.99-6.86 (m, 1H), 3.86 (s, 3H), 3.83-3.75 (m, 4H), 3.48 (s, 2H), 3.25-3.16 (m, 2H), 3.15-3.02 (m, 2H), 2.97-2.71 (m, 4H), 2.03 (s, 4H), 1.90-1.74 (m, 2H).

### 1-(5-Bromo-3,6-dihydropyridin-1(2H)-yl)-3-(1H-1,2,3-triazol-1-yl)propan-1-one (Intermediate OF)

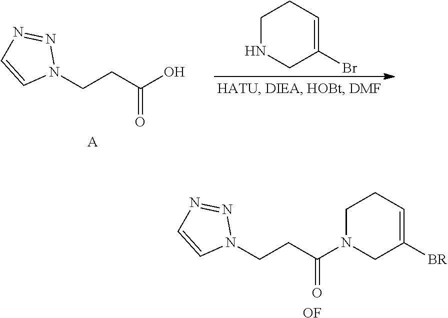

To a solution of 3-(triazol-1-yl)propanoic acid (2 g, 14.2 mmol, Intermediate A) and 5-bromo-1,2,3,6-tetrahydropyridine (2.30 g, 14.2 mmol) in DMF (35 mL) was added HATU (7.00 g, 18.4 mmol), HOBt (2.49 g, 18.4 mmol) and DIEA (9.16 g, 70.8 mmol, 12.3 mL). The mixture was then stirred at 25° C. for 1 hr. On completion, the mixture was quenched by addition of H₂O (50 mL), and then diluted with EA (50 mL) and extracted with EA (200 mL×3). The combined organic layers were washed with NaCl (100 mL×3), dried over Na₂SO₄, filtered and concentrated under reduced pressure to give a residue. The residue was purified by column chromatography (SiO₂, Petroleum ether/Ethyl acetate=20/1 to 0/1) to give the title compound (3.5 g, 87% yield) as a white solid. LC-MS (ESI+) m/z 284.8 (M+H)⁺.

### Tert-butyl 2-(7-fluoro-2-(4-(5-fluoro-3-methoxypyridin-2-yl)piperazine-1-carbonyl)-6-(4,4,5,5-tetramethyl-1,3,2-dioxaborolan-2-yl)-1H-indol-4-yl)piperidine-1-carboxylate (Intermediate OG)

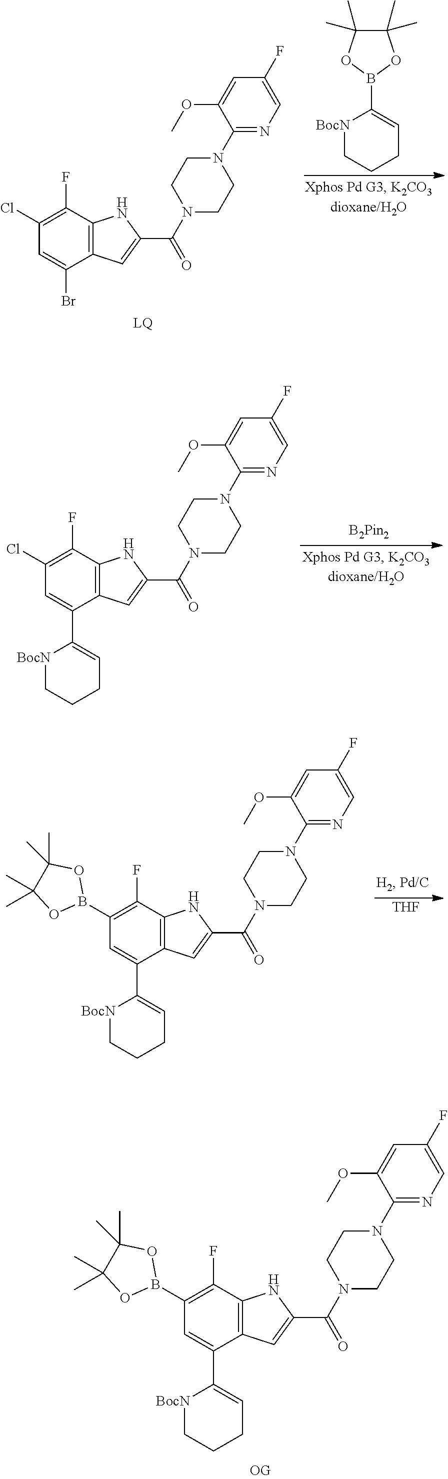

### Step 1—Tert-butyl 6-(6-chloro-7-fluoro-2-(4-(5-fluoro-3-methoxypyridin-2-yl)piperazine-1-carbonyl)-1H-indol-4-yl)-3,4-dihydropyridine-1(2H)-carboxylate

To a solution of (4-bromo-6-chloro-7-fluoro-1H-indol-2-yl)-[4-(5-fluoro-3-methoxy-2-pyridyl)piperazin-1-yl]methanone (1 g, 2 mmol, Intermediate LQ), tert-butyl 6-(4,4,5,5-tetramethyl-1,3,2-dioxaborolan-2-yl)-3,4-dihydro-2H-pyridine-1-carboxylate (637 mg, 2.06 mmol, CAS #865245-32-5) in dioxane (15 mL) and H₂O (4 mL) was added Pd(dppf)Cl₂·CH₂Cl₂ (168 mg, 206 μmol) and K₂CO₃ (854 mg, 6.18 mmol). The mixture was then stirred at 80° C. for 1 hr. On completion, the reaction mixture was quenched with H₂O (20 mL), and then diluted with EA (50 mL) and extracted with EA (50 mL×2). The combined organic layers were washed with NaCl (30 mL×2), dried over Na₂SO₄, filtered and concentrated under reduced pressure to give a residue. The crude product was purified by reversed-phase HPLC(0.1% FA condition) to give the title compound (300 mg, 25% yield) as a white solid. LC-MS (ESI+) m/z 588.1 (M+H)⁺.

### Step 2—Tert-butyl 6-(7-fluoro-2-(4-(5-fluoro-3-methoxypyridin-2-yl)piperazine-1-carbonyl)-6-(4,4,5,5-tetramethyl-1,3,2-dioxaborolan-2-yl)-1H-indol-4-yl)-3,4-dihydropyridine-1(2H)-carboxylate

To a solution of tert-butyl 6-[6-chloro-7-fluoro-2-[4-(5-fluoro-3-methoxy-2-pyridyl)piperazine-1-carbonyl]-1H-indol-4-yl]-3,4-dihydro-2H-pyridine-1-carboxylate (240 mg, 408 μmol) and 4,4,5,5-tetramethyl-2-(4,4,5,5-tetramethyl-1,3,2-dioxaborolan-2-yl)-1,3,2-dioxaborolane (104 mg, 408 μmol) in dioxane (8 mL) was added XPhos Pd G3 (34.5 mg, 40.8 μmol) and KOAc (120 mg, 1.22 mmol). The mixture was then stirred at 80° C. for 12 hrs. On completion, the mixture was quenched with H₂O (10 mL), and then diluted with EA (20 mL) and extracted with EA (30 mL×2). The combined organic layers were washed with NaCl (20 mL×2), dried over Na₂SO₄, filtered and concentrated under reduced pressure to give a residue. The residue was purified by column chromatography (SiO₂, Petroleum ether/Ethyl acetate=10/1 to 1/1) to give the title compound (0.3 g, 87% yield) as a white solid. LC-MS (ESI+) m/z 680.2 (M+H)⁺.

### Step 3—Tert-butyl 2-(7-fluoro-2-(4-(5-fluoro-3-methoxypyridin-2-yl)piperazine-1-carbonyl)-6-(4,4,5,5-tetramethyl-1,3,2-dioxaborolan-2-yl)-1H-indol-4-yl)piperidine-1-carboxylate

To a solution of tert-butyl 6-[7-fluoro-2-[4-(5-fluoro-3-methoxy-2-pyridyl)piperazine-1-carbonyl]-6-(4,4,5,5-tetramethyl-1,3,2-dioxaborolan-2-yl)-1H-indol-4-yl]-3,4-dihydro-2H-pyridine-1-carboxylate (250 mg, 368 μmol) in THE (15 mL) was added Pd/C (250 mg, 235 μmol, 10 wt %). Then the mixture was stirred at 25° C. for 12 hrs under H₂ (45 psi). On completion, the mixture was filtered then the filtrate was concentrated under reduced pressure to give a residue. The residue was purified by column chromatography (SiO₂, Petroleum ether/Ethyl acetate=10/1 to 2/1) to give the title compound (300 mg, 58% yield) as a white solid. LC-MS (ESI+) m/z 682.4 (M+H)⁺.

### (6-(1-(3-(1H-1,2,3-triazol-1-yl)propanoyl)-1,2,5,6-tetrahydropyridin-3-yl)-7-fluoro-2-(4-(3-methoxypyridin-2-yl)piperazine-1-carbonyl)-1H-indol-4-yl)boronic acid (Intermediate OH)

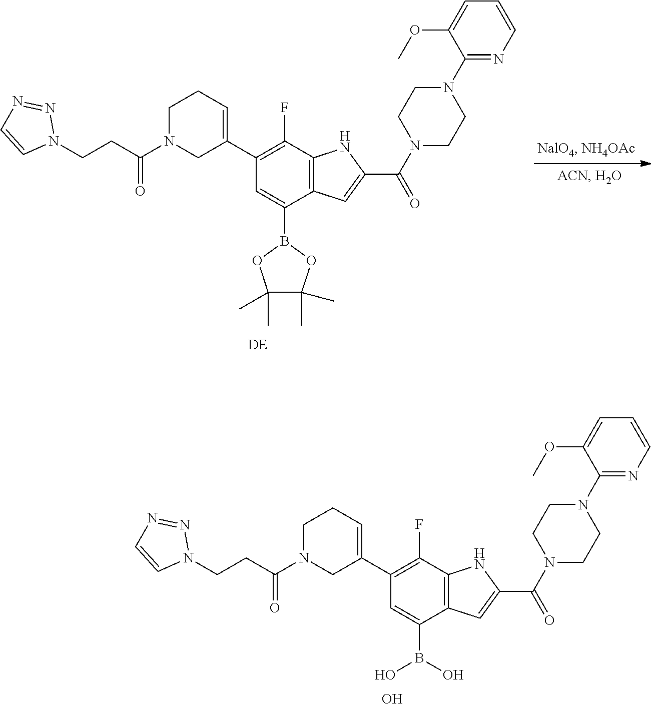

To a solution of 1-[5-[7-fluoro-2-[4-(3-methoxy-2-pyridyl)piperazine-1-carbonyl]-4-(4,4,5,5-tetramethyl-1,3,2-dioxaborolan-2-yl)-1H-indol-6-yl]-3,6-dihydro-2H-pyridin-1-yl]-3-(triazol-1-yl)propan-1-one (1 g, 2 mmol, Intermediate DE) in MeCN (20 mL) and H₂O (10 mL) was added NaIO₄ (937 mg, 4.38 mmol) and NH₄OAc (225 mg, 2.92 mmol). The mixture was then stirred at 25° C. for 12 hrs. On completion, the mixture was concentrated under reduced pressure to give a residue. The crude residue was purified by reversed-phase HPLC (0.1% FA condition) to afford the title compound (420 mg, 44% yield) as a brown solid. LC-MS (ESI⁺) m/z 633.3 (M+H)⁺; ¹H NMR (400 MHz, DMSO-d₆) δ=11.99-11.92 (m, 1H), 8.14-8.06 (m, 3H), 7.81 (dd, J=1.2, 4.8 Hz, 1H), 7.72-7.66 (m, 1H), 7.49 (d, J=7.2 Hz, 1H), 7.28 (dd, J=1.2, 8.0 Hz, 1H), 7.12 (s, 1H), 6.93 (dd, J=4.8, 8.0 Hz, 1H), 6.15-6.08 (m, 1H), 4.67-4.61 (m, 2H), 4.37-4.29 (m, 2H), 3.87-3.80 (m, 7H), 3.66 (t, J=6.0 Hz, 1H), 3.60 (t, J=5.6 Hz, 1H), 3.36 (s, 4H), 3.13-3.05 (m, 2H), 2.36 (s, 2H).

### 4-Cyclopropyl-1,2,3,6-tetrahydropyridine (Intermediate OI)

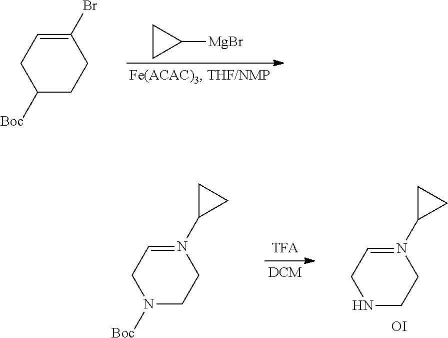

### Step 1—Tert-butyl 4-cyclopropyl-5,6-dihydropyridine-1(2H)-carboxylate

To a solution of tert-butyl 4-bromo-3,6-dihydro-2H-pyridine-1-carboxylate (500.00 mg, 1.91 mmol, CAS #159503-91-0) in THF (10 mL) and NMP (1 mL) was added Fe(ACAC)₃ (6.74 mg, 19.07 μmol) at −5° C., then bromo(cyclopropyl)magnesium (0.5 M, 4.96 mL, CAS #23719-80-4) was added and the reaction mixture was stirred at 20° C. for 12 h. On completion, the mixture was diluted with H₂O (200 mL) and extracted with EtOAc (100 mL×2). The combined organic layers were washed with brine (200 mL), dried over Na₂SO₄, filtered and concentrated under reduced pressure to give a residue. The residue was purified by flash silica gel chromatography (ISCO®; 80 g SepaFlash® Silica Flash Column, Eluent of 0˜100% EtOAc/PE gradient @100 mL/min) to give the title compound (200 mg, 38% yield) as a brown solid. ¹H NMR (400 MHz, CHLOROFORM-d) δ=5.37 (br s, 1H), 3.97-3.76 (m, 2H), 3.61-3.43 (m, 2H), 2.58-1.95 (m, 2H), 1.48-1.46 (m, 9H), 1.41-1.27 (m, 1H), 0.66-0.52 (m, 2H), 0.50-0.38 (m, 2H).

### Step 2—4-Cyclopropyl-1,2,3,6-tetrahydropyridine

To a solution of tert-butyl 4-cyclopropyl-3,6-dihydro-2H-pyridine-1-carboxylate (100.00 mg, 447.81 μmol) in DCM (3 mL) was added TFA (1.54 g, 13.5 mmol, 1 mL). The mixture was then stirred at 20° C. for 12 h. On completion, the mixture was concentrated under reduced pressure to give the title compound (55.00 mg) as a yellow oil. LC-MS (ESI+) m/z 124.2 (M+H)⁺.

### 1-Methyl-1,4,5,6-tetrahydropyrrolo[3,4-d]imidazole (Intermediate OJ)

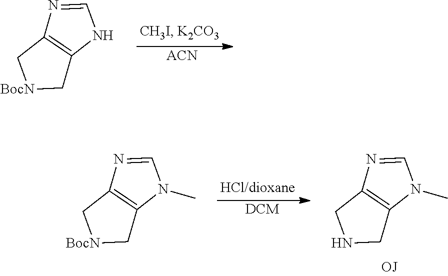

### Step 1—tert-butyl 1-methyl-4,6-dihydropyrrolo[3,4-d]imidazole-5(1H)-carboxylate

To a solution of tert-butyl 4,6-dihydro-1H-pyrrolo[3,4-d]imidazole-5-carboxylate (150.00 mg, 573.49 μmol, CAS #1050886-49-1) in THE (3.00 mL) was added NaH (57.34 mg, 1.43 mmol) and the mixture was stirred at 0° C. for 0.5 hr. Then CH₃I (81.40 mg, 573.49 μmol) was added and the mixture was stirred at 25° C. for 12 hr. Upon completion, water (10 mL) was added and the mixture was extracted with ethyl acetate (10 mL×3), then the organic phase was dried of sodium sulfate. The crude product was purified by reversed-phase HPLC(NH₄HCO₃) to give the title compound (125 mg, 97% yield) as a white solid. LC-MS (ESI⁺) m/z 224.2 (M+H)⁺.

### Step 2—1-Methyl-1,4,5,6-tetrahydropyrrolo[3,4-d]imidazole

A solution of tert-butyl 3-methyl-4,6-dihydropyrrolo[3,4-d]imidazole-5-carboxylate (90.00 mg, 403.1 μmol) in HCl/dioxane (2.00 mL) was stirred at 25° C. for 0.5 hr. Upon completion, the mixture was concentrated under reduced pressure to obtain the title compound (50.00 mg) as a yellow oil.

### Tert-butyl 1,3-dimethyl-1,4,5,6-tetrahydropyrrolo[3,4-c]pyrazole (Intermediate OK)

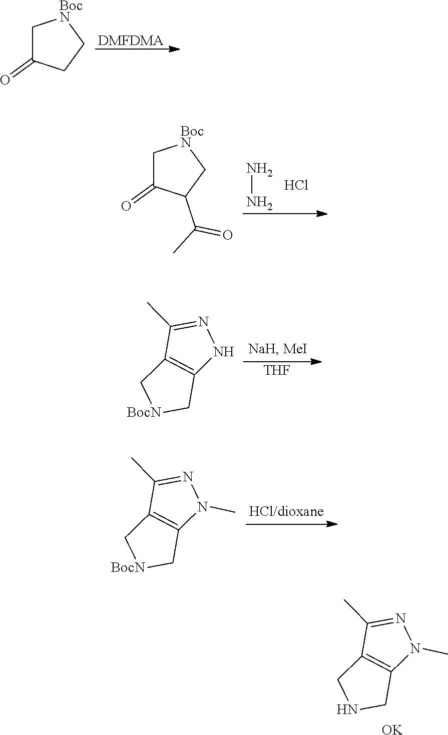

### Step 1—Tert-butyl 3-acetyl-4-oxopyrrolidine-1-carboxylate

To a solution of tert-butyl 3-oxopyrrolidine-1-carboxylate (5 g, 30 mmol, CAS #101385-93-7) in DMF-DMA (21 mL). The mixture was then stirred at 80° C. for 2 hrs. On completion, the mixture was quenched with NH₄Cl (30 mL) and extracted with EA (30 mL×3). The combined organic layers were washed with brine (20 mL×2), dried over Na₂SO₄, filtered and concentrated under reduced pressure to give a residue to afford the title compound (6 g, 79% yield) as a orange solid. ¹H NMR (400 MHz, CDCl₃-d) δ=7.31 (s, 1H), 4.61-4.50 (m, 2H), 3.88-3.79 (m, 2H), 3.09 (s, 6H), 1.50-1.47 (m, 9H)

### Step 2—Tert-butyl 3-methyl-4,6-dihydropyrrolo[3,4-c]pyrazole-5(1H)-carboxylate

To a solution of tert-butyl 3-acetyl-4-oxo-pyrrolidine-1-carboxylate (500 mg, 2.20 mmol) in AcOH (4 mL) was added hydrazine hydrochloride (226.09 mg, 3.30 mmol). The mixture was then stirred at 25° C. for 3 hrs. On completion, the mixture was diluted with H₂O (10 mL) and extracted with EA (10 mL×3). The combined organic layers were washed with NaHCO₃ (10 mL×2), dried over Na₂SO₄, filtered and concentrated under reduced pressure to give the title compound (350 mg, 57% yield) as a deep yellow oil. ¹H NMR (400 MHz, CHLOROFORM-d) δ=4.38-4.19 (m, 4H), 2.18 (d, J=5.6 Hz, 3H), 1.40 (s, 9H).

### Step 3—Tert-butyl 1,3-dimethyl-4,6-dihydropyrrolo[3,4-c]pyrazole-5(1H)-carboxylate

To a solution of tert-butyl 3-methyl-4,6-dihydro-1H-pyrrolo[3,4-c]pyrazole-5-carboxylate (250 mg, 1.12 mmol) in THF (3 mL) was added NaH (44.78 mg, 1.12 mmol, 60% dispersion in mineral oil) and MeI (190 mg, 1.34 mmol). The mixture was then stirred at 0-25° C. for 3 hrs. On completion, the mixture was quenched with NH₄Cl (10 mL) at 0° C., and then extracted with EA (20 mL×3). The combined organic layers were dried over Na₂SO₄, filtered and concentrated under reduced pressure to give a residue. The residue was purified by prep-HPLC (column: CD01-Phenomenex luna C18 150*25*10 um;mobile phase: [water(TFA)-ACN];gradient:28%-48% B over 10 min) to give the title compound (90 mg, 23% yield, TFA) as a white solid. LC-MS (ESI⁺) m/z 238.2 (M+H)⁺; ¹H NMR (400 MHz, DMSO-d6) δ=4.40 (d, J=12.8 Hz, 2H), 4.23 (d, J=13.6 Hz, 2H), 3.65 (d, J=2.8 Hz, 3H), 2.07 (d, J=3.2 Hz, 3H), 1.44 (d, J=0.8 Hz, 9H).

### Step 4—Tert-butyl 1,3-dimethyl-1,4,5,6-tetrahydropyrrolo[3,4-c]pyrazole

To a solution of tert-butyl 1,3-dimethyl-4,6-dihydropyrrolo[3,4-c]pyrazole-5-carboxylate (80 mg, 337 μmol) in HCl/dioxane (2 M, 2 mL). The mixture was then stirred at 25° C. for 3 hrs. On completion, the mixture was filtered and concentrated under reduced pressure to give the title compound (50 mg, HCl) as a yellow oil.

### Tert-butyl 5-ethyl-4-(((trifluoromethyl)sulfonyl)oxy)-5,6-dihydropyridine-1(2H)-carboxylate (Intermediate OL)

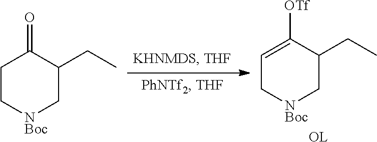

To a solution of tert-butyl 3-ethyl-4-oxo-piperidine-1-carboxylate (1 g, 4 mmol, CAS #117565-57-8) in THF (40 mL) was added KHMDS (1 M, 5.28 mL) slowly at 0° C. The mixture was then stirred for 2 hrs at 0° C. Next, 1,1,1-trifluoro-N-phenyl-N-(trifluoromethylsulfonyl)methanesulfonamide (2.36 g, 6.60 mmol, CAS #37595-74-7) in THF (4 mL) was added and the mixture was stirred at 0-25° C. for 6 hrs. Upon completion, the mixture was quenched with NH₄Cl (20 mL) at 0° C., and then extracted with EA (30 mL×3). The combined organic layers were washed with brine (20 mL×2), dried over Na₂SO₄, filtered and concentrated under reduced pressure to give a residue to afford the title compound (1.3 g, 82% yield) as a colorless oil. ¹H NMR (400 MHz, CDCl₃-d) δ=5.82-5.63 (m, 1H), 4.14 (d, J=7.2 Hz, 2H), 3.60 (s, 1H), 1.77-1.70 (m, 2H), 1.62 (d, J=6.4 Hz, 2H), 0.90-0.85 (m, 12H).

### Tert-butyl 3-ethyl-4-(7-fluoro-2-(4-(5-fluoro-3-methoxypyridin-2-yl)piperazine-1-carbonyl)-6-(4,4,5,5-tetramethyl-1,3,2-dioxaborolan-2-yl)-1H-indol-4-yl)piperidine-1-carboxylate (Intermediate OM)

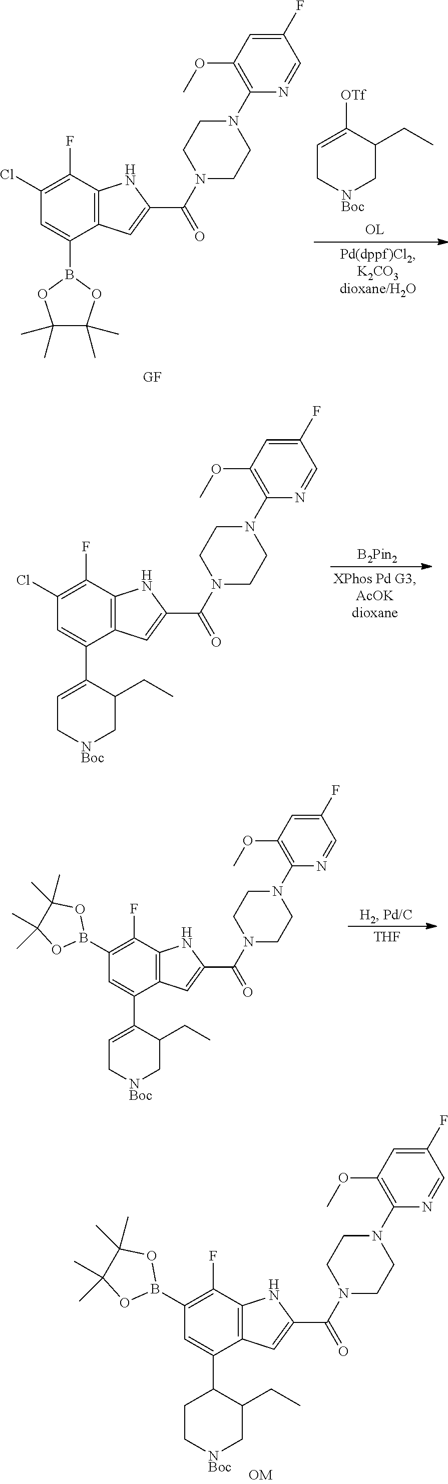 [SMILES: COc1cc(F)cnc1N1CCN(C(=O)c2cc3c(B4OC(C)(C)C(C)(C)O4)cc(Cl)c(F)c3[nH]2)CC1 ; CCC1CN(C(=O)OC(C)(C)C)CC=C1OS(=O)(=O)C(F)(F)F ; CCC1CN(C(=O)OC(C)(C)C)CC=C1c1cc(Cl)c(F)c2[nH]c(C(=O)N3CCN(c4ncc(F)cc4OC)CC3)cc12 ; "THF" ; CCC1CN(C(=O)OC(C)(C)C)CC=C1c1cc(B2OC(C)(C)C(C)(C)O2)c(F)c2[nH]c(C(=O)N3CCN(c4ncc(F)cc4OC)CC3)cc12 ; CCC1CN(C(=O)OC(C)(C)C)CCC1c1cc(B2OC(C)(C)C(C)(C)O2)c(F)c2[nH]c(C(=O)N3CCN(c4ncc(F)cc4OC)CC3)cc12]

### Step 1—Tert-butyl 4-(6-chloro-7-fluoro-2-(4-(5-fluoro-3-methoxypyridin-2-yl)piperazine-1-carbonyl)-1H-indol-4-yl)-5-ethyl-5,6-dihydropyridine-1(2H)-carboxylate

To a solution of tert-butyl 3-ethyl-4-(trifluoromethylsulfonyloxy)-3,6-dihydro-2H-pyridine-1-carboxylate (1.21 g, 3.38 mmol, Intermediate OL) and [6-chloro-7-fluoro-4-(4,4,5,5-tetramethyl-1,3,2-dioxaborolan-2-yl)-1H-indol-2-yl]-[4-(5-fluoro-3-methoxy-2-pyridyl)piperazin-1-yl]methanone (600 mg, 1.13 mmol, Intermediate GF) in dioxane (6 mL) and H₂O (1 mL) was added Pd(dppf)Cl₂·CH₂Cl₂ (91.9 mg, 112 μmol) and K₂CO₃ (466 mg, 3.38 mmol). The mixture was then stirred at 80° C. for 2 hrs under N₂ atmosphere. Upon completion, the mixture was filtered and concentrated under reduced pressure to give a residue. The residue was purified by column chromatography (SiO₂, Petroleum ether/Ethyl acetate=50/1 to 2/1) to give the title compound (600 mg, 73% yield) as a white solid. LC-MS (ESI⁺) m/z 616.2 (M+H)⁺; ¹H NMR (400 MHz, CDCl₃-d) δ=9.35 (s, 1H), 7.70 (d, J=2.4 Hz, 1H), 7.32 (d, J=8.0 Hz, 1H), 7.22 (d, J=7.6 Hz, 1H), 6.89-6.83 (m, 2H), 3.98 (s, 4H), 3.84 (s, 3H), 3.37 (t, J=4.8 Hz, 4H), 1.50 (s, 4H), 1.45 (s, 9H), 1.29-1.20 (m, 2H), 1.17 (s, 3H), 0.81 (d, J=4.4 Hz, 3H).

### Step 2—Tert-butyl 5-ethyl-4-(7-fluoro-2-(4-(5-fluoro-3-methoxypyridin-2-yl)piperazine-1-carbonyl)-6-(4,4,5,5-tetramethyl-1,3,2-dioxaborolan-2-yl)-1H-indol-4-yl)-5,6-dihydropyridine-1(2H)-carboxylate

To a solution of tert-butyl 4-[6-chloro-7-fluoro-2-[4-(5-fluoro-3-methoxy-2-pyridyl)piperazine-1-carbonyl]-1H-indol-4-yl]-3-ethyl-3,6-dihydro-2H-pyridine-1-carboxylate (600 mg, 973 μmol) and 4,4,5,5-tetramethyl-2-(4,4,5,5-tetramethyl-1,3,2-dioxaborolan-2-yl)-1,3,2-dioxaborolane (2.47 g, 9.74 mmol) in dioxane (10 mL) was added XPhos Pd G3 (82.4 mg, 97.3 μmol) and KOAc (286 mg, 2.92 mmol). The mixture was then stirred at 80° C. for 12 hrs under N₂ atmosphere. Upon completion, the mixture was filtered and concentrated under reduced pressure to give a residue. The residue was purified by prep-HPLC (FA condition) to give the title compound (150 mg, 21% yield) as a white solid. ¹H NMR (400 MHz, CDCl₃-d) δ=9.26-9.18 (m, 1H), 7.70 (d, J=2.0 Hz, 1H), 7.14 (d, J=4.4 Hz, 1H), 7.01 (d, J=4.4 Hz, 1H), 6.86 (td, J=2.4, 9.6 Hz, 1H), 6.71 (s, 1H), 4.05-3.92 (m, 4H), 3.85 (s, 3H), 3.43-3.36 (m, 6H), 1.50 (s, 9H), 1.45 (s, 5H), 1.42 (s, 3H), 1.32 (s, 12H).

### Step 3—Tert-butyl 3-ethyl-4-(7-fluoro-2-(4-(5-fluoro-3-methoxypyridin-2-yl)piperazine-1-carbonyl)-6-(4,4,5,5-tetramethyl-1,3,2-dioxaborolan-2-yl)-1H-indol-4-yl)piperidine-1-carboxylate

To a solution of tert-butyl 3-ethyl-4-[7-fluoro-2-[4-(5-fluoro-3-methoxy-2-pyridyl)piperazine-1-carbonyl]-6-(4,4,5,5-tetramethyl-1,3,2-dioxaborolan-2-yl)-1H-indol-4-yl]-3,6-dihydro-2H-pyridine-1-carboxylate (150 mg, 211 μmol) in THF (2 mL) was added Pd/C (150 mg, 140 mol, 10 wt %). The mixture was stirred at 25° C. for 12 hrs under H₂ (50 psi). Upon completion, the mixture was filtered and concentrated under reduced pressure to give the title compound (120 mg, 44% yield) as a white solid. LC-MS (ESI⁺) m/z 710.5 (M+H)⁺.

### (4-(4-Ethylpyridin-3-yl)-7-fluoro-6-(1,2,5,6-tetrahydropyridin-3-yl)-1H-indol-2-yl)(4-(5-fluoro-3-methoxypyridin-2-yl)piperazin-1-yl)methanone (Intermediate ON)

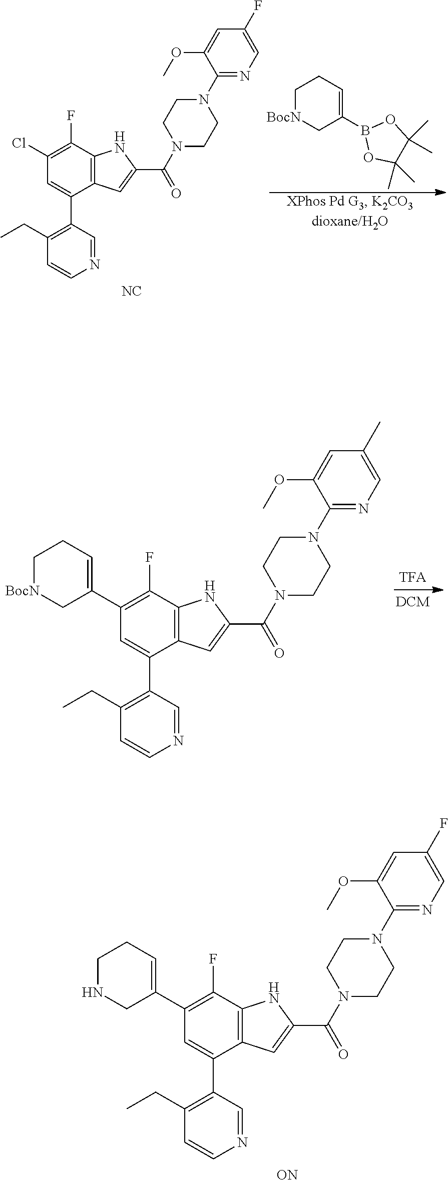

### Step 1—Tert-butyl 3-(4-(4-ethylpyridin-3-yl)-7-fluoro-2-(4-(3-methoxy-5-methylpyridin-2-yl)piperazine-1-carbonyl)-1H-indol-6-yl)-5,6-dihydropyridine-1(2H)-carboxylate

To a solution of [6-chloro-4-(4-ethyl-3-pyridyl)-7-fluoro-1H-indol-2-yl]-[4-(5-fluoro-3-methoxy-2-pyridyl)piperazin1-yl]methanone (400 mg, 781 μmol, Intermediate NC) and tert-butyl 5-(4,4,5,5-tetramethyl-1,3,2-dioxaborolan-2-yl)-3,6-dihydro-2H-pyridine-1-carboxylate (362.39 mg, 1.17 mmol, CAS #885693-20-9) in dioxane (8 mL) and H₂O (1.6 mL) were added XPhos Pd G3 (66.14 mg, 78.13 μmol) and K₂CO₃ (323.95 mg, 2.34 mmol). The mixture was then stirred at 80° C. for 1 hr. Upon completion, the reaction mixture was quenched with H₂O (10 mL), and then extracted with ethyl acetate (50 mL). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 0˜8% MeOH/DCM gradient @60 mL/min) to give the title compound (460 mg, 89% yield) as a yellow solid. LC-MS (ESI⁺) m/z 659.3 (M+H)⁺.

### Step 2—(4-(4-Ethylpyridin-3-yl)-7-fluoro-6-(1,2,5,6-tetrahydropyridin-3-yl)-1H-indol-2-yl)(4-(5-fluoro-3-methoxypyridin-2-yl)piperazin-1-yl)methanone

To a solution of tert-butyl 5-[4-(4-ethyl-3-pyridyl)-7-fluoro-2-[4-(5-fluoro-3-methoxy-2-pyridyl)piperazine-1-carbonyl]-1H-indol-6-yl]-3,6-dihydro-2H-pyridine-1-carboxylate (360 mg, 547 mol) in DCM (3 mL) was added TFA (2.76 g, 24.2 mmol, 1.80 mL). The mixture was then stirred at 20° C. for 1 hr. On completion, the reaction mixture was concentrated under reduced pressure to give the title compound (410 mg, TFA) as a yellow oil. LC-MS (ESI⁺) m/z 559.3 (M+H)⁺.

### Tert-butyl 3-ethyl-4-(trifluoromethylsulfonyloxy)-2,5-dihydropyrrole-1-carboxylate (Intermediate OO)

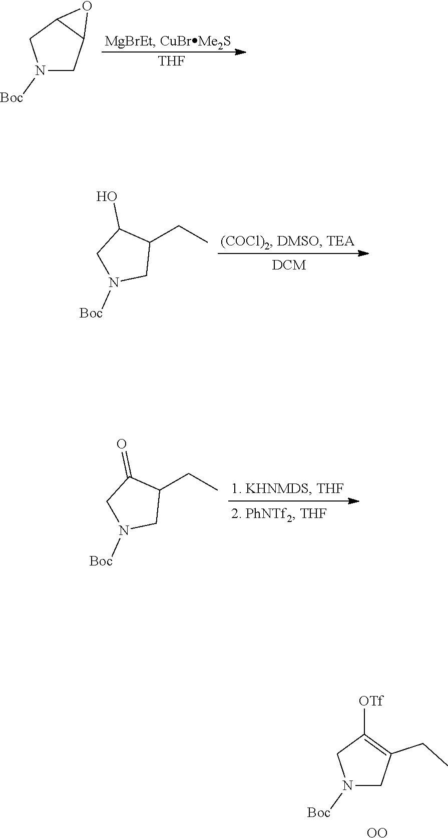

### Step 1—Tert-butyl 3-ethyl-4-hydroxy-pyrrolidine-1-carboxylate

To a solution of tert-butyl 6-oxa-3-azabicyclo[3.1.0]hexane-3-carboxylate (8 g, 40 mmol, CAS #114214-49-2) and CuBrMe₂S (1.78 g, 8.64 mmol) in THF (80 mL) was dropwise added EtMgBr (3 M, 17.28 mL) at −30° C. under N₂ atmosphere, and then the mixture was stirred at −30° C. for 2 hrs under N₂ atmosphere. On completion, the reaction mixture was quenched with NH₄Cl (100 mL) and extracted with EtOAc (50 mL×2). The combined organic layers were dried over Na₂SO₄, filtered and concentrated under reduced pressure to give a residue. The residue was purified by flash silica gel chromatography (ISCO®; 80 g SepaFlash® Silica Flash Column, Eluent of 0˜30% EtOAc/PE gradient @100 mL/min) to give the title compound (7.5 g, 77% yield) as a white gum. ¹H NMR (400 MHz, DMSO-d₆) δ=4.98 (d, J=4.4 Hz, 1H), 3.86-3.77 (m, 1H), 3.46-3.36 (m, 2H), 2.98 (td, J=4.4, 10.8 Hz, 1H), 2.93-2.81 (m, 1H), 1.79 (dt, J=6.8, 13.2 Hz, 1H), 1.48-1.40 (m, 2H), 1.38 (s, 9H), 0.88 (t, J=7.2 Hz, 3H).

### Step 2—Tert-butyl 3-ethyl-4-oxo-pyrrolidine-1-carboxylate

To a solution of (COCl)₂ (17.69 g, 139.4 mmol, 12.20 mL) in DCM (50 mL) was added DMSO (10.89 g, 139.4 mmol, 10.89 mL) as a solution in DCM (10 mL) at −78° C. under N₂ atmosphere, the mixture was stirred at −78° C. for 1 hr. Then tert-butyl 3-ethyl-4-hydroxy-pyrrolidine-1-carboxylate (15 g, 69.67 mmol) as a solution in DCM (10 mL) was added to the mixture dropwise and the mixture was stirred at −78° C. for 2 hr. Next, TEA (28.20 g, 278.70 mmol, 38.79 mL) was added to the mixture and stirred at −78° C. for 1 hr. Upon completion, the mixture was diluted with saturated solution of NaHCO₃ (500 mL) and extracted with DCM (200 mL×2). The combined organic layers were dried over Na₂SO₄, filtered and concentrated under reduced pressure to give a residue. The residue was purified by flash silica gel chromatography (ISCO®; 80 g SepaFlash® Silica Flash Column, Eluent of 0˜30% EtOAc/PE gradient @100 mL/min) to give the title compound (12 g, 73% yield) as a yellow gum. ¹H NMR (400 MHz, DMSO-d₆) δ=3.91 (t, J=10.0 Hz, 1H), 3.84-3.72 (m, 1H), 3.60 (br d, J=17.6 Hz, 1H), 3.20 (br s, 1H), 2.59 (br s, 1H), 1.73-1.59 (m, 1H), 1.42 (br s, 9H), 1.39-1.29 (m, 1H), 0.91-0.84 (m, 3H).

### Step 3—Tert-butyl 3-ethyl-4-(trifluoromethylsulfonyloxy)-2,5-dihydropyrrole-1-carboxylate

To a solution of tert-butyl 3-ethyl-4-oxo-pyrrolidine-1-carboxylate (5 g, 20 mmol) in anhydrous THF (100 mL) was added KHMDS (1 M, 28.13 mL) dropwise at 50° C. and then the mixture was stirred at 50° C. for 2 hrs under N₂ atmosphere. Then a solution of 1,1,1-trifluoro-N-phenyl-N-(trifluoromethylsulfonyl)methanesulfonamide (9.21 g, 25.8 mmol) in THF (20 mL) was added to the mixture at 50° C. and the mixture was stirred at 50° C. for 4 h under N₂ atmosphere. On completion, the reaction mixture was quenched with NH₄Cl (300 mL) and extracted with EtOAc (200 mL×2). The combined organic layers were dried over Na₂SO₄, filtered and concentrated under reduced pressure to give a residue. The residue was purified by flash silica gel chromatography (ISCO®; 120 g SepaFlash® Silica Flash Column, Eluent of 0˜10% EtOA/PE gradient @100 mL/min) to give the title compound (1.5 g, 19% yield) as a yellow gum. ¹H NMR (400 MHz, DMSO-d₆) δ=4.22-4.14 (m, 2H), 4.10 (br dd, J=4.0, 13.2 Hz, 2H), 2.26-2.12 (m, 2H), 1.41 (d, J=1.6 Hz, 9H), 1.00 (t, J=7.6 Hz, 3H).

### Tert-butyl 5-ethyl-4-(trifluoromethylsulfonyloxy)-3,6-dihydro-2H-pyridine-1-carboxylate (Intermediate OP)

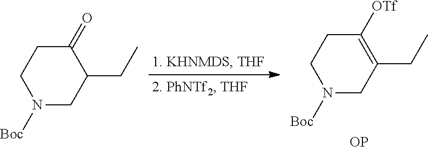

To a solution of tert-butyl 3-ethyl-4-oxo-piperidine-1-carboxylate (5 g, 20 mmol, CAS #117565-57-8) in anhydrous THF (100 mL) was added KHMDS (1 M, 26.40 mL) dropwise at 50° C. and then the mixture was stirred at 50° C. for 6 hrs under N₂ atmosphere. Then a solution of 1,1,1-trifluoro-N-phenyl-N-(trifluoromethylsulfonyl)methanesulfonamide (10.22 g, 28.60 mmol) in THF (20 mL) was added to the mixture at 50° C. and the mixture was stirred at 50° C. for 6 hrs under N₂ atmosphere. On completion, the reaction mixture was quenched with NH₄Cl (300 mL) and extracted with EtOAc (200 mL×2). The combined organic layers were dried over Na₂SO₄, filtered and concentrated under reduced pressure to give a residue. The residue was purified by flash silica gel chromatography (ISCO®; 40 g SepaFlash® Silica Flash Column, Eluent of 0˜20% EtOAc/PE gradient @100 mL/min) to give the title compound (12 g, 15% yield) as a yellow gum. ¹H NMR (400 MHz, DMSO-d₆) δ=3.96 (br s, 2H), 3.57-3.49 (m, 2H), 2.42-2.33 (m, 2H), 2.16 (q, J=7.6 Hz, 2H), 1.41 (s, 9H), 1.01 (br t, J=7.6 Hz, 3H).

### 5-Bromo-4-ethyl-1-methyl-1,2,3,6-tetrahydropyridine (Intermediate OO)

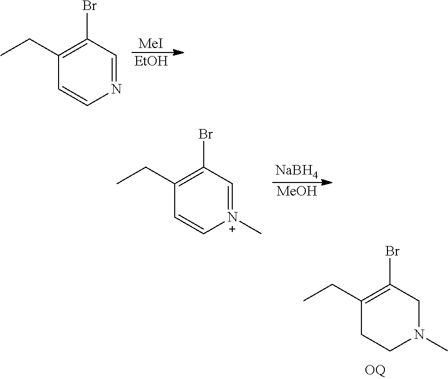

### Step 1—3-Bromo-4-ethyl-1-methylpyridin-1-ium

To a solution of 3-bromo-4-ethyl-pyridine (0.8 g, 4.3 mmol, CAS #38749-76-7) in EtOH (25 mL) was added MeI (3.05 g, 21.5 mmol). The mixture was then stirred at 92° C. for 16 hrs under N₂ atmosphere. Upon completion, the mixture was filtered and concentrated under reduced pressure to give a residue to afford the title compound (2 g, 93% yield) as a brown oil. ¹H NMR (400 MHz, CDCl₃-d) δ=9.34-9.13 (m, 2H), 7.97 (d, J=6.4 Hz, 1H), 4.68 (s, 3H), 3.02 (q, J=7.6 Hz, 2H), 1.39 (t, J=7.6 Hz, 3H).

### Step 2—5-Bromo-4-ethyl-1-methyl-1,2,3,6-tetrahydropyridine

To a solution of 3-bromo-4-ethyl-1-methyl-pyridin-1-ium (1 g, 5 mmol) in MeOH (10 mL) was added NaBH₄ (940 mg, 24.8 mmol) at 0° C. slowly. The mixture was stirred at 0-25° C. for 2 hrs. Upon completion, the mixture was quenched with H₂O (50 mL) at 0° C., and then extracted with EA (50 mL×3). The combined organic layers were washed with brine (50 mL×2), dried over Na₂SO₄, filtered and concentrated under reduced pressure to give a residue. The residue was purified by column chromatography (SiO₂, DCM/Ethyl acetate=1:0 to 10:1) to give the title compound (80 mg, 8% yield) as an off yellow oil. ¹H NMR (400 MHz, CDCl₃-d) δ=3.77 (d, J=17.2 Hz, 1H), 3.31 (d, J=16.8 Hz, 1H), 3.05-2.88 (m, 2H), 2.56 (s, 3H), 2.32-2.25 (m, 2H), 2.21 (quin, J=7.2 Hz, 2H), 0.98 (t, J=7.6 Hz, 3H).

### (7-Bromo-1-(cyclopropylmethyl)-2-(1,2,5,6-tetrahydropyridin-3-yl)-1H-indol-5-yl)(4-(5-fluoro-3-methoxypyridin-2-yl)piperazin-1-yl)methanone (Intermediate OS)

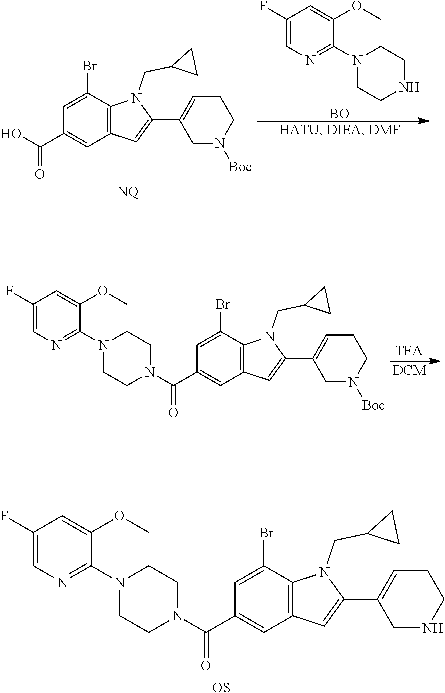

### Step 1—Tert-butyl 3-(7-bromo-1-(cyclopropylmethyl)-5-(4-(5-fluoro-3-methoxypyridin-2-yl)piperazine-1-carbonyl)-1H-indol-2-yl)-5,6-dihydropyridine-1(2H)-carboxylate

To a solution of 7-bromo-2-(1-(tert-butoxycarbonyl)-1,2,5,6-tetrahydropyridin-3-yl)-1-(cyclopropylmethyl)-1H-indole-5-carboxylic acid (0.5 g, 770 μmol, Intermediate NQ) in DMF (5 mL) was added HATU (379 mg, 998 μmol), DIEA (496 mg, 3.84 mmol, 668 μL) and 1-(5-fluoro-3-methoxypyridin-2-yl)piperazine (194 mg, 921 μmol, Intermediate BO). The mixture was then stirred at 25° C. for 0.5 hr. On completion, the reaction mixture was triturated with H₂O at 0° C. for 15 min and filtered to give the title compound (0.5 g) as a white solid. LC-MS (ESI+) m/z 670.1 (M+H)⁺.

### Step 2—(7-Bromo-1-(cyclopropylmethyl)-2-(1,2,5,6-tetrahydropyridin-3-yl)-1H-indol-5-yl)(4-(5-fluoro-3-methoxypyridin-2-yl)piperazin-1-yl)methanone

To a solution of tert-butyl 3-(7-bromo-1-(cyclopropylmethyl)-5-(4-(5-fluoro-3-methoxypyridin-2-yl)piperazine-1-carbonyl)-1H-indol-2-yl)-5,6-dihydropyridine-1(2H)-carboxylate (0.47 g, 702 μmol) in DCM (5 mL) was added TFA (80.1 mg, 702 μmol, 52 μL). The mixture was then stirred at 25° C. for 2 hrs. On completion, the reaction mixture was concentrated in vacuo to give the title compound (0.4 g, TFA) as a red solid. LC-MS (ESI+) m/z 570.0 (M+H)⁺.

### 1-(3-(7-Bromo-1-(cyclopropylmethyl)-5-(4-(5-fluoro-3-methoxypyridin-2-yl)piperazine-1-carbonyl)-1H-indol-2-yl)-5,6-dihydropyridin-1(2H)-yl)-3-(1H-1,2,3-triazol-1-yl)propan-1-one (Intermediate OT)

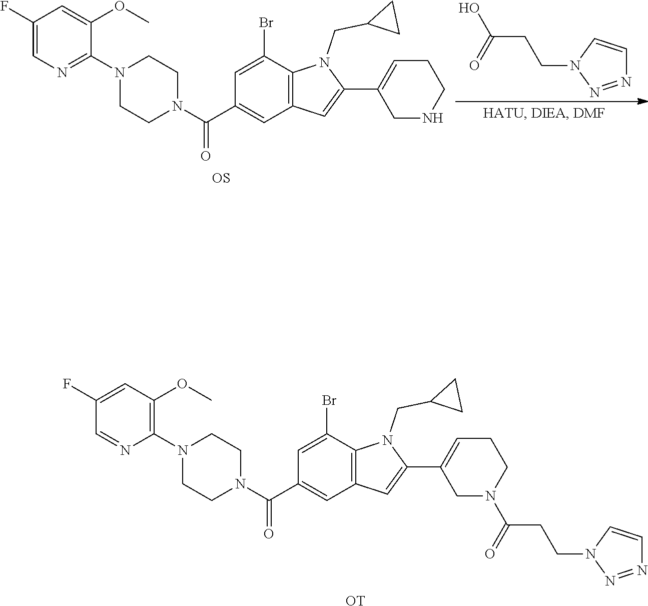

To a solution of 3-(1H-1,2,3-triazol-1-yl)propanoic acid (124 mg, 879 μmol, CAS #4332-45-0) in DMF (5 mL) was added HATU (289 mg, 761 μmol), DIEA (530 mg, 4.10 mmol, 714 μL) and (7-bromo-1-(cyclopropylmethyl)-2-(1,2,5,6-tetrahydropyridin-3-yl)-1H-indol-5-yl)(4-(5-fluoro-3-methoxypyridin-2-yl)piperazin-1-yl)methanone (0.4 g, 586 μmol, TFA, Intermediate OS). The mixture was then stirred at 25° C. for 2 hrs. On completion, the mixture was triturated with H₂O at 0° C. for 15 min, then filtered to give the title compound (0.4 g) as a white solid. LC-MS (ESI+) m/z 692.9 (M+H)⁺.

### 4-Ethyl-3-(4,45,5-tetramethyl-1,3,2-dioxaborolan-2-yl)pyridine (Intermediate OU)

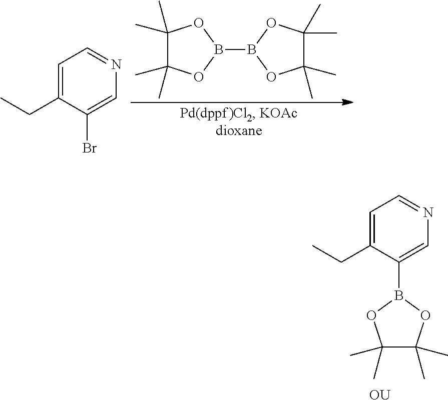

A mixture of 3-bromo-4-ethyl-pyridine (500 mg, 2.69 mmol, CAS #38749-76-7), 4,4,5,5-tetramethyl-2-(4,4,5,5-tetramethyl-1,3,2-dioxaborolan-2-yl)-1,3,2-dioxaborolane (5.46 g, 21.5 mmol), Pd(dppf)Cl₂·CH₂Cl₂ (219 mg, 268 μmol) and KOAc (527 mg, 5.37 mmol) in dioxane (10 mL) was degassed and purged with N₂ three times. Then the mixture was stirred at 90° C. for 12 hrs under N₂ atmosphere. Upon completion, the mixture was filtered and concentrated under reduced pressure to give a residue. The residue was purified by column chromatography (SiO₂, Petroleum ether/Ethyl acetate=50/1 to 10/1) to afford the title compound (300 mg, 40% yield) as a white solid. ¹H NMR (400 MHz, DMSO-d₆) δ=8.66 (s, 1H), 8.51 (d, J=5.2 Hz, 1H), 7.24 (d, J=5.2 Hz, 1H), 2.82 (q, J=7.6 Hz, 2H), 1.31 (s, 12H), 1.15-1.11 (m, 3H).

### (4-Ethyl-3-pyridyl)boronic acid (CAS #1001907-67-0) (Intermediate OV)

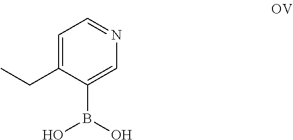

### (2-Ethyl-3-pyridyl)boronic acid (CAS #1310384-02-1) (Intermediate OW)

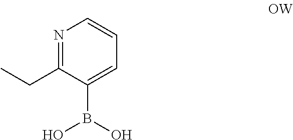

### 3-Chloro-2-ethyl-6-methylpyridine (Intermediate OX)

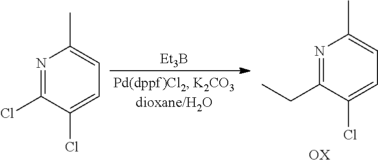

To a solution of 2,3-dichloro-6-methyl-pyridine (1 g, 6 mmol, CAS #54957-86-7) and triethylborane (1 M, 7.41 mL, CAS #97-94-9) in dioxane (10 mL) and H₂O (2 mL) was added K₂CO₃ (2.56 g, 18.5 mmol) and Pd(dppf)Cl₂ (451 mg, 617 μmol). The mixture was stirred at 65° C. for 12 hrs under N₂ atmosphere. Upon completion, the mixture was filtered and concentrated under reduced pressure to give a residue. The residue was purified by column chromatography (SiO₂, Petroleum ether/Ethyl acetate=1/0 to 1/1) to afford the title compound (700 mg, 66% yield) as a deep yellow oil. LC-MS (ESI⁺) m/z 156.1 (M+H)⁺; ¹H NMR (400 MHz, DMSO-d6) δ=7.76 (d, J=8.0 Hz, 1H), 7.17 (d, J=8.0 Hz, 1H), 2.88 (q, J=7.6 Hz, 2H), 2.49 (s, 3H), 1.25 (t, J=7.6 Hz, 3H).

### Tert-butyl 3-(5-(4-(5-fluoro-3-methoxypyridin-2-yl)piperazine-1-carbonyl)-7-(4,4,5,5-tetramethyl-1,3,2-dioxaborolan-2-yl)-1H-indol-2-yl)-5,6-dihydropyridine-1(2H)-carboxylate (Intermediate OY)

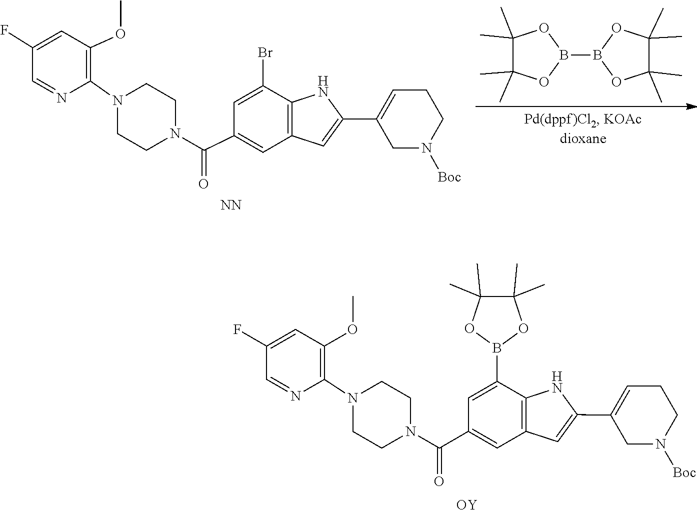

A mixture of tert-butyl 3-(7-bromo-5-(4-(5-fluoro-3-methoxypyridin-2-yl)piperazine-1-carbonyl)-1H-indol-2-yl)-5,6-dihydropyridine-1(2H)-carboxylate (3.1 g, 5.0 mmol, Intermediate NN), 4,4,5,5-tetramethyl-2-(4,4,5,5-tetramethyl-1,3,2-dioxaborolan-2-yl)-1,3,2-dioxaborolane (3.84 g, 15.1 mmol), KOAc (1.49 g, 15.1 mmol), and Pd(dppf)Cl₂·CH₂Cl₂ (412 mg, 504 μmol) in dioxane (60 mL) was degassed and purged with N₂ three times. Then the mixture was stirred at 80° C. for 12 hrs under N₂ atmosphere. On completion, the reaction mixture was diluted with water (200 mL) and filtered. The filter cake was concentrated in vacuo to give the crude residue. The residue was purified by column chromatography (SiO₂, Petroleum ether:Ethyl acetate=10/1 to 1/1) to give the title compound (3 g, 86% yield) as a yellow solid. LC-MS (ESI+) m/z 662.4 (M+H)⁺; ¹H NMR (400 MHz, DMSO-d6) δ=9.65 (s, 1H), 7.77 (dd, J=2.0, 12.4 Hz, 2H), 7.47 (d, J=1.6 Hz, 1H), 7.39-7.31 (m, 1H), 6.57 (d, J=1.6 Hz, 1H), 6.44 (br s, 1H), 4.27 (br s, 2H), 3.92 (br s, 2H), 3.83 (s, 3H), 3.65 (br d, J=1.6 Hz, 2H), 3.53-3.49 (m, 2H), 3.23 (br s, 4H), 2.35-2.31 (m, 2H), 1.44 (s, 9H), 1.37 (s, 12H).

### Tert-butyl 3-(7-(2-ethyl-6-methylpyridin-3-yl)-5-(4-(5-fluoro-3-methoxypyridin-2-yl)piperazine-1-carbonyl)-1H-indol-2-yl)-5,6-dihydropyridine-1(2H)-carboxylate (Intermediate OZ)

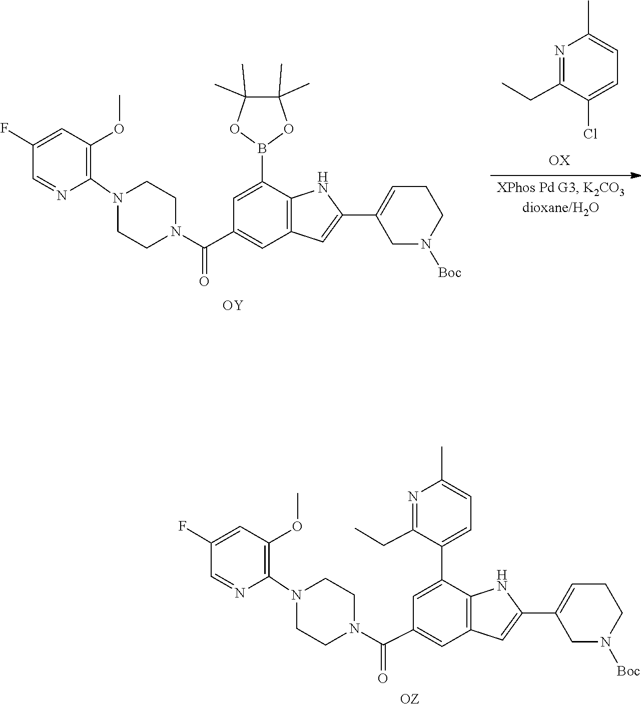

A mixture of tert-butyl 3-(5-(4-(5-fluoro-3-methoxypyridin-2-yl)piperazine-1-carbonyl)-7-(4,4,5,5-tetramethyl-1,3,2-dioxaborolan-2-yl)-1H-indol-2-yl)-5,6-dihydropyridine-1(2H)-carboxylate (2.98 g, 4.50 mmol, Intermediate OY), 3-chloro-2-ethyl-6-methylpyridine (700 mg, 4.50 mmol, Intermediate OX), XPhos Pd G3 (381 mg, 450 μmol), and K₂CO₃ (1.86 g, 13.5 mmol) in dioxane (10 mL) and H₂O (2 mL) was degassed and purged with N₂ three times. Then the mixture was stirred at 80° C. for 2 hrs under N₂ atmosphere. On completion, the reaction mixture was diluted with water (200 mL) and filtered. The filter cake was dried in vacuo to give the crude residue. The residue was purified by column chromatography (SiO₂, Petroleum ether:Ethyl acetate=1/1 to 0/1) to give the title compound (1.8 g, 60% yield) as a yellow solid. LC-MS (ESI+) m/z 655.3 (M+H)⁺; ¹H NMR (400 MHz, DMSO-d6) δ=10.81 (s, 1H), 7.78 (d, J=2.4 Hz, 1H), 7.63 (s, 1H), 7.50 (d, J=8.0 Hz, 1H), 7.35 (dd, J=2.4, 10.4 Hz, 1H), 7.18 (d, J=7.6 Hz, 1H), 6.91 (d, J=1.2 Hz, 1H), 6.58 (br d, J=19.2 Hz, 2H), 4.23 (br s, 2H), 3.83 (s, 3H), 3.66 (br s, 4H), 3.46 (br t, J=5.2 Hz, 2H), 3.31-3.28 (m, 2H), 3.24 (br s, 4H), 2.55-2.54 (m, 3H), 2.23 (br s, 2H), 1.43 (s, 9H), 1.02 (t, J=7.6 Hz, 3H).

### (7-(2-Ethyl-6-methylpyridin-3-yl)-1-isobutyl-2-(1,2,5,6-tetrahydropyridin-3-yl)-1H-indol-5-yl)(4-(5-fluoro-3-methoxypyridin-2-yl)piperazin-1-yl)methanone (Intermediate PA)

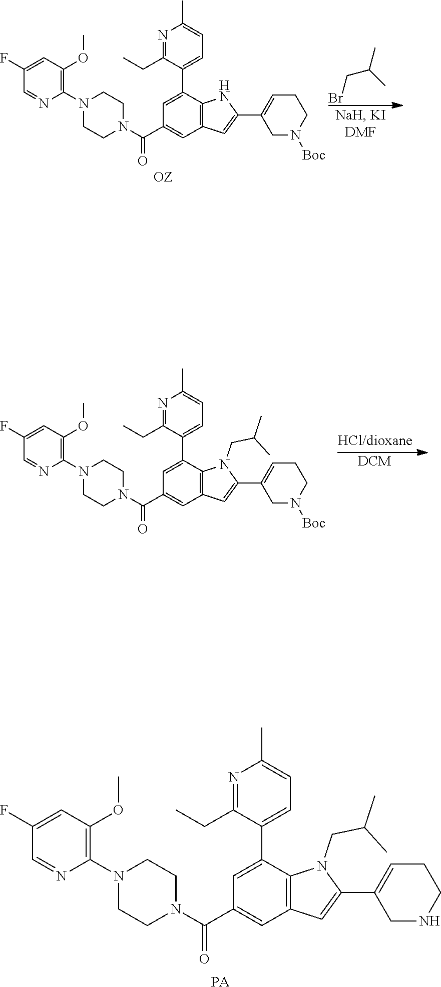

### Step 1—Tert-butyl 3-(7-(2-ethyl-6-methylpyridin-3-yl)-5-(4-(5-fluoro-3-methoxypyridin-2-yl)piperazine-1-carbonyl)-1-isobutyl-1H-indol-2-yl)-5,6-dihydropyridine-1(2H)-carboxylate

To a solution of tert-butyl 3-(7-(2-ethyl-6-methylpyridin-3-yl)-5-(4-(5-fluoro-3-methoxypyridin-2-yl)piperazine-1-carbonyl)-1H-indol-2-yl)-5,6-dihydropyridine-1(2H)-carboxylate (260 mg, 397 μmol, Intermediate OZ) in DMF (3 mL) was added NaH (47.7 mg, 1.19 mmol, 60% dispersion in mineral oil). Then 1-bromo-2-methylpropane (81.6 mg, 596 μmol) and KI (6.59 mg, 39.7 μmol) was added at 0° C. and the mixture was stirred at 0-25° C. for 12 hrs. On completion, the reaction mixture was quenched with ice water (10 mL) at 0° C. and extracted with ethyl acetate (3×20 mL). The combined organic layers were washed with brine (10 mL) and dried over anhydrous sodium sulfate, filtered and the filtrate was concentrated in vacuo to give the crude residue. The residue was purified by column chromatography (SiO₂, Petroleum ether:Ethyl acetate=10/1 to 1/1) to give the title compound (80 mg, 18% yield) as a yellow oil. LC-MS (ESI+) m/z 711.4 (M+H)⁺.

### Step 2—(7-(2-Ethyl-6-methylpyridin-3-yl)-1-isobutyl-2-(1,2,5,6-tetrahydropyridin-3-yl)-1H-indol-5-yl)(4-(5-fluoro-3-methoxypyridin-2-yl)piperazin-1-yl)methanone

To a solution of tert-butyl 3-(7-(2-ethyl-6-methylpyridin-3-yl)-5-(4-(5-fluoro-3-methoxypyridin-2-yl)piperazine-1-carbonyl)-1-isobutyl-1H-indol-2-yl)-5,6-dihydropyridine-1(2H)-carboxylate (80 mg, 113 μmol) in DCM (1 mL) was added HCl/dioxane (2 M, 1 mL). The mixture was then stirred at 25° C. for 0.5 hr. On completion, the reaction mixture was filtered and the filter cake was dried in vacuo to give the title compound (70 mg, HCl) as a yellow solid. LC-MS (ESI+) m/z 611.4 (M+H)⁺.

### (1—(Cyclopropylmethyl)-7-(2-ethyl-6-methylpyridin-3-yl)-2-(1,2,5,6-tetrahydropyridin-3-yl)-1H-indol-5-yl)(4-(5-fluoro-3-methoxypyridin-2-yl)piperazin-1-yl)methanone (Intermediate PB)

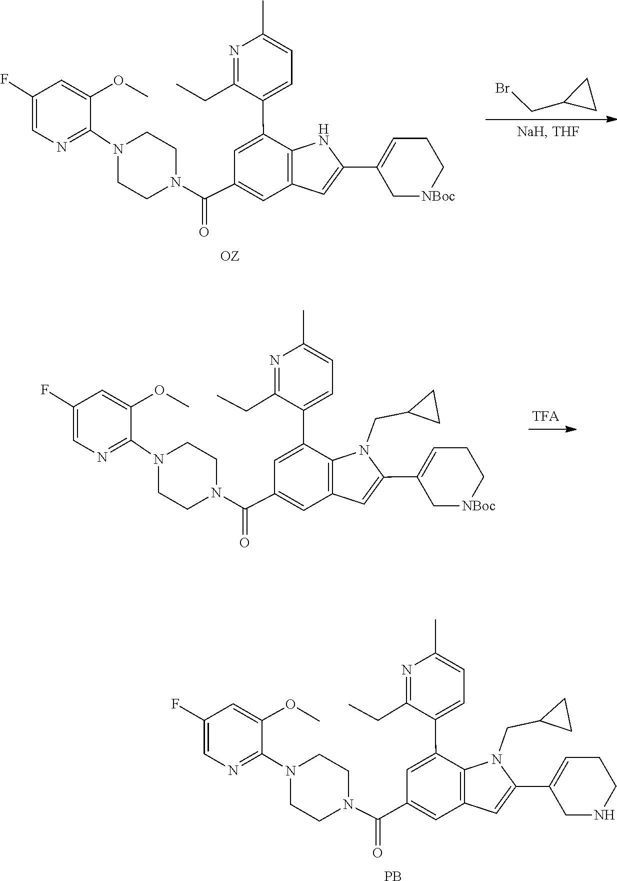

### Step 1—Tert-butyl 3-(1-(cyclopropylmethyl)-7-(2-ethyl-6-methylpyridin-3-yl)-5-(4-(5-fluoro-3-methoxypyridin-2-yl)piperazine-1-carbonyl)-1H-indol-2-yl)-5,6-dihydropyridine-1(2H)-carboxylate

To a solution of tert-butyl 5-[7-(2-ethyl-6-methyl-3-pyridyl)-5-[4-(5-fluoro-3-methoxy-2-pyridyl)piperazine-1-carbonyl]-1H-indol-2-yl]-3,6-dihydro-2H-pyridine-1-carboxylate (230 mg, 351 μmol, Intermediate OZ) in THF (3 mL) was added NaH (21.07 mg, 526 μmol, 60% dispersion in mineral oil) at 0° C. and bromomethylcyclopropane (94.8 mg, 702 μmol, CAS #7051-34-5). The mixture was then stirred at 25° C. for 2 hrs. Upon completion, the mixture was quenched with NH₄Cl (2 mL) at 0° C., and then extracted with EA (20 mL×3). The combined organic layers were dried over Na₂SO₄, filtered and concentrated under reduced pressure to give a residue. The residue was purified by column chromatography (SiO2, DCM:MeOH=10:1) to give the title compound (180 mg, 58% yield) as a yellow oil. LC-MS (ESI⁺) m/z 709.5 (M+H)⁺; ¹H NMR (400 MHz, DMSO-d6) δ=8.01 (d, J=2.4 Hz, 1H), 7.95-7.87 (m, 2H), 7.58 (dd, J=2.4, 10.4 Hz, 1H), 7.44 (d, J=7.6 Hz, 1H), 7.13 (d, J=1.6 Hz, 1H), 6.86 (s, 1H), 6.20 (s, 1H), 5.98 (s, 1H), 4.35-4.23 (m, 2H), 4.06 (s, 3H), 3.88 (d, J=0.8 Hz, 4H), 3.76-3.58 (m, 4H), 3.46 (s, 4H), 3.39 (d, J=5.2 Hz, 4H), 2.77 (s, 3H), 2.64 (d, J=7.6 Hz, 2H), 2.51-2.45 (m, 2H), 1.63 (s, 9H), 1.40 (t, J=7.2 Hz, 2H), 1.30-1.19 (m, 4H).

### Step 2—(1—(Cyclopropylmethyl)-7-(2-ethyl-6-methylpyridin-3-yl)-2-(1,2,5,6-tetrahydropyridin-3-yl)-1H-indol-5-yl)(4-(5-fluoro-3-methoxypyridin-2-yl)piperazin-1-yl)methanone

To a solution of tert-butyl 5-[1-(cyclopropylmethyl)-7-(2-ethyl-6-methyl-3-pyridyl)-5-[4-(5-fluoro-3-methoxy-2-pyridyl)piperazine-1-carbonyl]indol-2-yl]-3,6-dihydro-2H-pyridine-1-carboxylate (180 mg, 253 μmol) was added TFA (2 mL). The mixture was then stirred at 25° C. for 1 hr. Upon completion, the mixture was filtered and concentrated under reduced pressure to give the title compound (200 mg, TFA) as an orange oil. LC-MS (ESI⁺) m/z 609.3 (M+H)⁺.

### 1-(5-(7-Fluoro-2-(4-(5-fluoro-3-methoxy-6-methylpyridin-2-yl)piperazine-1-carbonyl)-4-(4,4,5,5-tetramethyl-1,3,2-dioxaborolan-2-yl)-1H-indol-6-yl)-3,6-dihydropyridin-1(2H)-yl)-3-(1H-1,2,3-triazol-1-yl)propan-1-one (Intermediate PC)

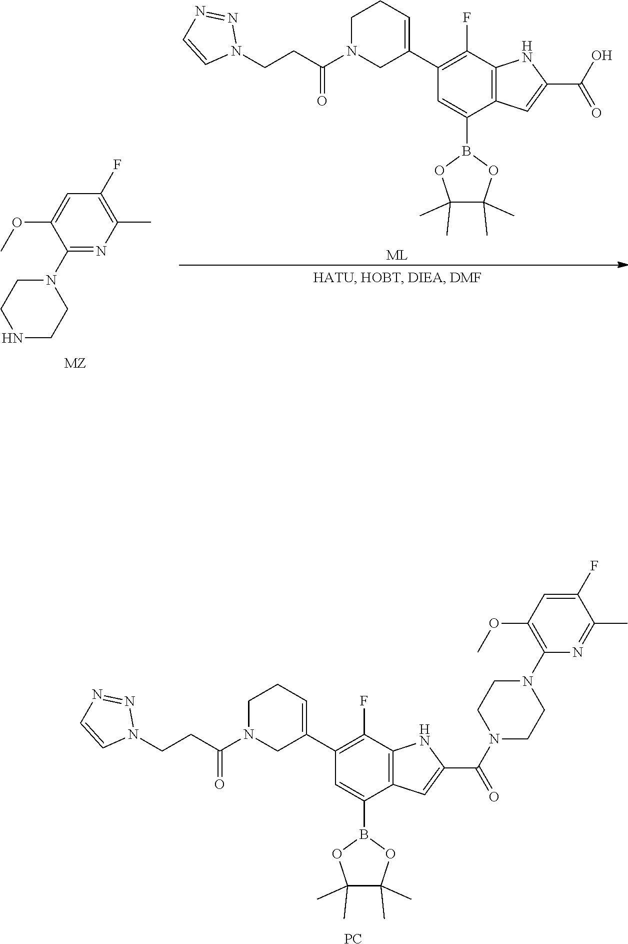

To a solution of 7-fluoro-4-(4,4,5,5-tetramethyl-1,3,2-dioxaborolan-2-yl)-6-[1-[3-(triazol-1-yl)propanoyl]-3,6-dihydro-2H-pyridin-5-yl]-1H-indole-2-carboxylic acid (500 mg, 982 μmol, Intermediate ML) in DMF (3.00 mL) was added HOBt (265 mg, 1.96 mmol), DIEA (634. mg, 4.91 mmol, 854.94 μL) and 1-(5-fluoro-3-methoxy-6-methyl-2-pyridyl)piperazine (221 mg, 844.91 μmol, HCl, Intermediate MZ). Then HATU (448 mg, 1.18 mmol) was added and the mixture was stirred at 25° C. for 0.5 hr. Upon completion, to the mixture was added H₂O (10 mL) at 0° C., and then the mixture was filtered and concentrated under reduced pressure to give the title compound (480 mg, 68% yield) as a yellow solid. LC-MS (ESI⁺) m/z 717.4 (M+H)⁺; ¹H NMR (400 MHz, DMSO-d₆) δ=12.18-12.11 (m, 1H), 8.13-8.10 (m, 1H), 7.95 (s, 1H), 7.70-7.67 (m, 1H), 7.30 (d, J=10.8 Hz, 1H), 6.99 (s, 1H), 6.05 (s, 1H), 4.68-4.55 (m, 4H), 4.41-4.25 (m, 4H), 3.81 (s, 4H), 3.77 (d, J=3.6 Hz, 6H), 2.89 (s, 3H), 2.73 (s, 3H), 1.33 (s, 12H).

### 5-Chloro-4-ethyl-2-methyl-pyridine (Intermediate PD)

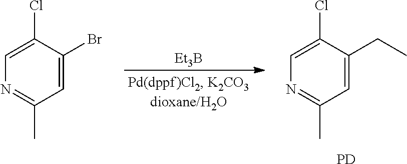

To a solution of 4-bromo-5-chloro-2-methyl-pyridine (500 mg, 2.42 mmol, CAS #1211529-34-8) and triethylborane (1 M, 2.50 mL) in dioxane (5 mL) and H₂O (0.5 mL) was added K₂CO₃ (1.00 g, 7.27 mmol) and Pd(dppf)Cl₂ (88.60 mg, 121.08 μmol). The mixture was then stirred at 65° C. for 1 hr under N₂ atmosphere. On completion, the reaction mixture was filtered and the filtrate was concentrated under reduced pressure to give a residue. The residue was purified by column chromatography (SiO₂, Petroleum ether/Ethyl acetate=10/1 to 5/1) to give the title compound (840 mg, 67% yield) as a colorless oil. ¹H NMR (400 MHz, DMSO-d) δ=8.38 (s, 1H), 7.26 (s, 1H), 2.67 (q, J=7.6 Hz, 2H), 2.43 (s, 3H), 1.17 (t, J=7.6 Hz, 3H).

### 4-(4-Ethyl-6-methyl-3-pyridyl)-7-fluoro-6-[1-(3-pyrazol-1-ylpropanoyl)-3,6-dihydro-2H-pyridin-5-yl]-1H-indole-2-carboxylic acid (Intermediate PE)

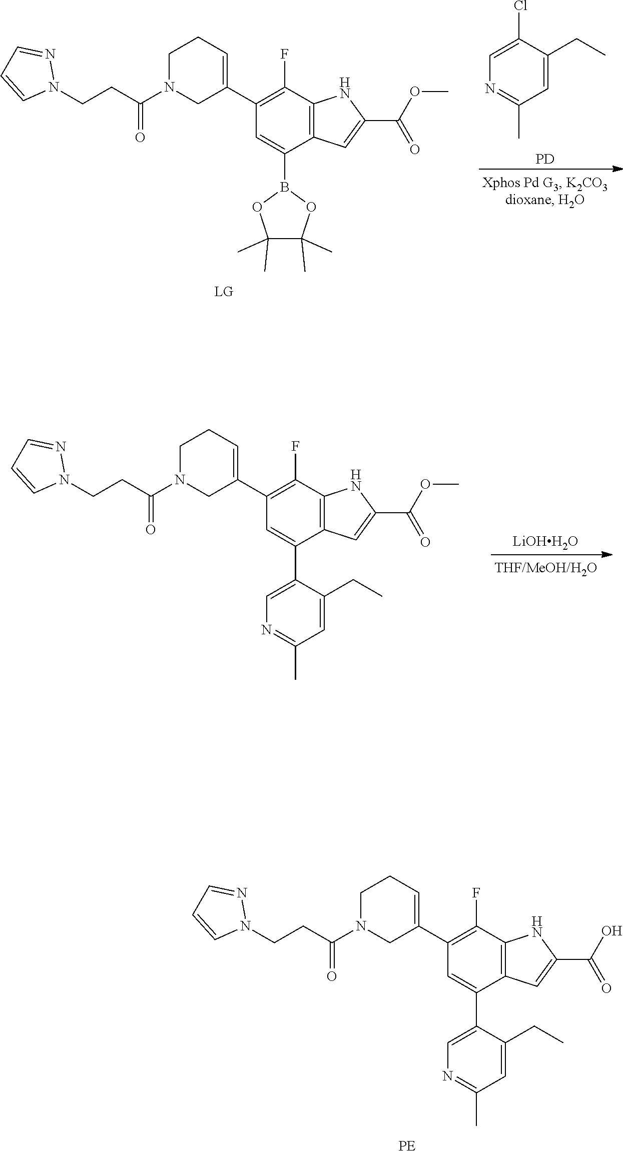

### Step 1—Methyl 4-(4-ethyl-6-methyl-3-pyridyl)-7-fluoro-6-[1-(3-pyrazol-1-ylpropanoyl)-3,6-dihydro-2H-pyridin-5-yl]-1H-indole-2-carboxylate

A mixture of methyl 7-fluoro-6-[1-(3-pyrazol-1-ylpropanoyl)-3,6-dihydro-2H-pyridin-5-yl]-4-(4,4,5,5-tetramethyl-1,3,2-dioxaphospholan-2-yl)-1H-indole-2-carboxylate (105 mg, 193 μmol, Intermediate LG), 5-chloro-4-ethyl-2-methyl-pyridine (30 mg, 190 μmol, Intermediate PD), XPhos Pd G3 (16 mg, 19 μmol), and K₂CO₃ (80 mg, 578.32 μmol) in dioxane (5 mL) and H₂O (1 mL) was degassed and purged with N₂ three times. Then the mixture was stirred at 80° C. for 2 h under N₂ atmosphere. On completion, the mixture was extracted with EtOAc (60 mL). The combined organic extracts were washed with saturated brine (20 mL) and then dried over Na₂SO₄, filtered and concentrated under vacuum. The residue was purified by column chromatography (SiO₂, PE/EtOAc=10/1 to 0/1) to give the title compound (150 mg, 79% yield) as a yellow solid. LC-MS (ESI⁺) m/z 516.2 (M+H)⁺; ¹H NMR (400 MHz, DMSO-d₆) δ=12.68 (s, 1H), 8.24 (s, 1H), 7.73-7.68 (m, 1H), 7.29 (s, 1H), 6.99-6.87 (m, 1H), 6.68 (d, J=3.2 Hz, 1H), 6.27-6.07 (m, 3H), 4.36 (s, 2H), 4.31 (d, J=11.2 Hz, 1H), 4.09 (q, J=5.2 Hz, 1H), 4.00 (s, 1H), 3.84 (s, 3H), 3.67-3.61 (m, 1H), 3.59-3.56 (m, 1H), 3.17 (d, J=5.2 Hz, 3H), 2.54 (s, 3H), 1.99 (s, 2H), 0.97 (t, J=7.6 Hz, 3H).

### Step 2—4-(4-Ethyl-6-methyl-3-pyridyl)-7-fluoro-6-[1-(3-pyrazol-1-ylpropanoyl)-3,6-dihydro-2H-pyridin-5-yl]-1H-indole-2-carboxylic acid

To a solution of methyl 4-(4-ethyl-6-methyl-3-pyridyl)-7-fluoro-6-[1-(3-pyrazol-1-ylpropanoyl)-3,6-dihydro-2H-pyridin-5-yl]-1H-indole-2-carboxylate (200 mg, 388 μmol) in MeOH (2 mL), THF (4 mL) and H₂O (1 mL) was added LiOH·H₂O (81 mg, 1.94 mmol). The mixture was then stirred at 20° C. for 0.5 h. On completion, the reaction mixture was quenched with HCl (6 mL 2 M), and then extracted with DCM (150 mL). The combined organic layers were dried over Na₂SO₄, filtered and concentrated under reduced pressure to give the title compound (200 mg, 98% yield) as a yellow solid. LC-MS (ESI⁺) m/z 502.2 (M+H)⁺.

### 5-Chloro-4-isopropyl-2-methyl-pyridine (Intermediate PF)

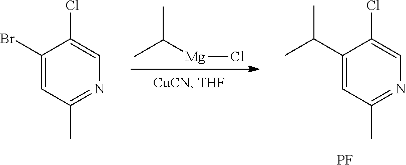

To a solution of 4-bromo-5-chloro-2-methyl-pyridine (400.00 mg, 1.94 mmol, CAS #1211529-34-8) in THF (10.00 mL) was added CuCN (17.35 mg, 193.7 μmol) at 20° C. Then chloro(isopropyl)magnesium (2.0 M, 1.26 mL, CAS #1068-55-9) was added to the mixture and the mixture was stirred at 20° C. for 12 hr. Upon completion, the mixture was poured into sat NH₄Cl solution (10 mL) at 0° C., then the mixture was extracted with EtOAc (20 mL×2). The combined organic layers were dried, filtered and concentrated under vacuum. The residue was purified by column chromatography (SiO₂, Petroleum ether/Ethyl acetate=20/1 to 5/1) to give the title compound (120 mg, 32% yield) as a yellow oil. LC-MS (ESI⁺) m/z 170.0 (M+H)⁺; ¹H NMR (400 MHz, CDCl₃) δ (ppm)=8.38 (s, 1H), 7.06 (s, 1H), 3.46-3.12 (m, 1H), 2.52 (s, 3H), 1.25 (d, J=6.8 Hz, 6H).

### 1-(2-Methoxy-5-methyl-phenyl)piperazine (CAS #189264-72-0) (Intermediate PG)

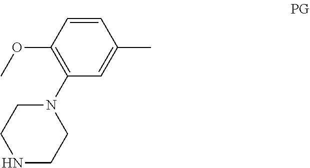

### 4-(4-Ethyl-6-methylpyridin-3-yl)-7-fluoro-6-(1,2,5,6-tetrahydropyridin-3-yl)-1H-indol-2-yl)(1-ethylpyrrolo[3,4-c]pyrazol-5(1H,4H,6H)-yl)methanone (Intermediate PH)

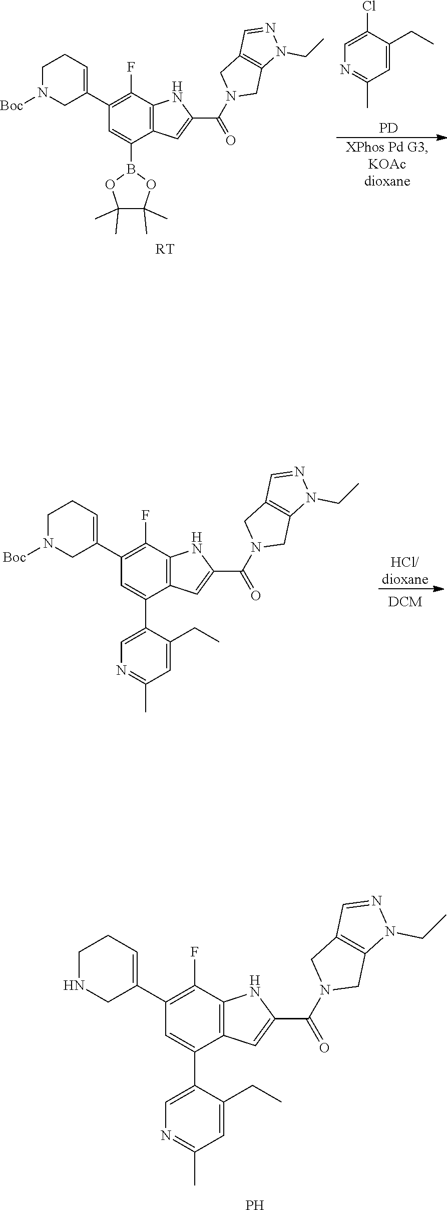

### Step 1—Tert-butyl 3-(2-(1-ethyl-1,4,5,6-tetrahydropyrrolo[3,4-c]pyrazole-5-carbonyl)-4-(4-ethyl-6-methylpyridin-3-yl)-7-fluoro-1H-indol-6-yl)-5,6-dihydropyridine-1(2H)-carboxylate

To a solution of tert-butyl 5-[2-(1-ethyl-4,6-dihydropyrrolo[3,4-c]pyrazole-5-carbonyl)-7-fluoro-4-(4,4,5,5-tetramethyl-1,3,2-dioxaborolan-2-yl)-1H-indol-6-yl]-3,6-dihydro-2H-pyridine-1-carboxylate (250.00 mg, 412.88 μmol, Intermediate RT) and 5-chloro-4-ethyl-2-methyl-pyridine (64.25 mg, 412.9 μmol, Intermediate PD) in H₂O (0.8 mL) and dioxane (3.5 mL) were added XPhos Pd G₃ (34.95 mg, 41.29 μmol) and KOAc (121.56 mg, 1.24 mmol). The mixture was then stirred at 100° C. for 1 h. Upon completion, the mixture was diluted with H₂O (2 mL) and extracted with EtOAc (5 mL×2). The combined organic layers were dried over Na₂SO₄, filtered and concentrated under reduced pressure to give a residue. The residue was purified by flash silica gel chromatography (ISCO®; 4 g SepaFlash® Silica Flash Column, Eluent of 0˜100% EtOAc/Petroleum ether gradient @60 mL/min) to give the title compound (150.00 mg, 54% yield) as a yellow solid. LC-MS (ESI+) m/z 599.3 (M+H)⁺.

### Step 2—(4-(4-Ethyl-6-methylpyridin-3-yl)-7-fluoro-6-(1,2,5,6-tetrahydropyridin-3-yl)-1H-indol-2-yl)(1-ethylpyrrolo[3,4-c]pyrazol-5(1H,4H,6H)-yl)methanone

To a solution of tert-butyl 3-(2-(1-ethyl-1,4,5,6-tetrahydropyrrolo[3,4-c]pyrazole-5-carbonyl)-4-(4-ethyl-6-methylpyridin-3-yl)-7-fluoro-1H-indol-6-yl)-5,6-dihydropyridine-1(2H)-carboxylate (150.00 mg, 250.54 μmol) in DCM (2 mL) was added HCl/dioxane (2 M, 3.14 mL). The mixture was then stirred at 20° C. for 1 h. Upon completion, the mixture was concentrated under reduced pressure to give the title compound (118.00 mg, 85% yield) as a white solid. LC-MS (ESI⁺) m/z 499.3 (M+H)⁺; ¹H NMR (400 MHz, CHLOROFORM-d) δ=9.76-9.39 (m, 1H), 8.42 (s, 1H), 7.22-7.17 (m, 1H), 6.90 (d, J=6.0 Hz, 1H), 6.61 (d, J=2.8 Hz, 1H), 6.15 (s, 1H), 4.85 (d, J=16.4 Hz, 4H), 4.17 (q, J=7.6 Hz, 2H), 3.77 (s, 2H), 3.08 (t, J=6.0 Hz, 2H), 2.65 (s, 2H), 2.58-2.44 (m, 2H), 2.31 (d, J=3.6 Hz, 2H), 1.52-1.48 (m, 3H), 1.08 (t, J=7.6 Hz, 3H), 0.90-0.83 (m, 3H).

### 4-(4-Ethyl-6-methyl-3-pyridyl)-7-fluoro-6-[1-[3-(triazol-1-yl)propanoyl]-3,6-dihydro-2H-pyridin-5-yl]-1H-indole-2-carboxylic acid (Intermediate PI)

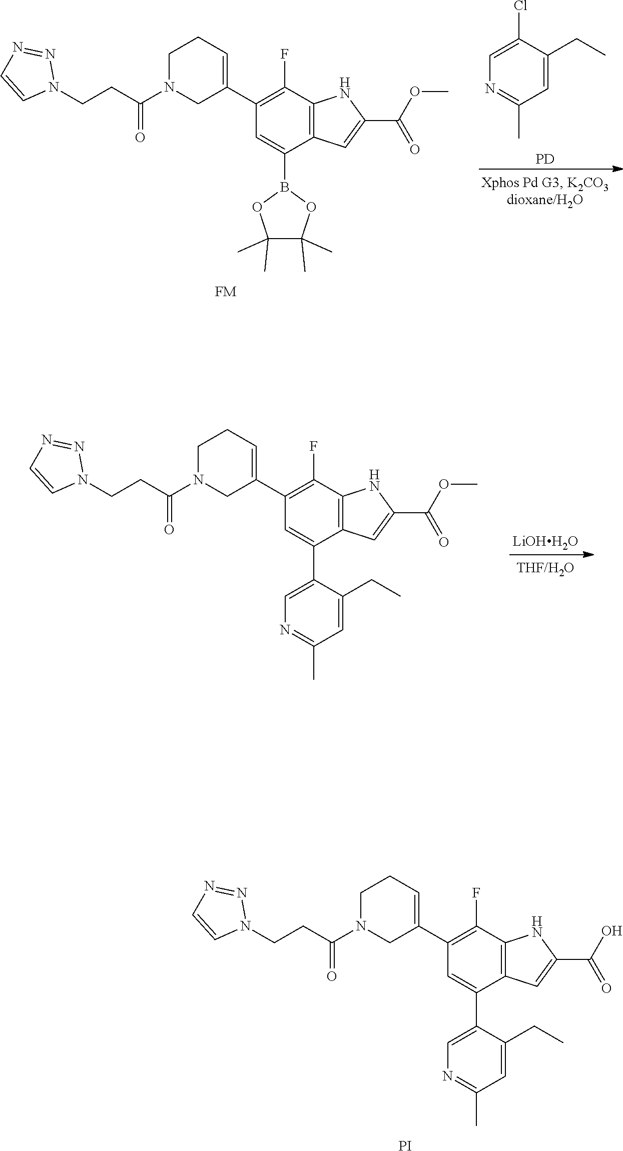

### Step 1—Methyl 4-(4-ethyl-6-methyl-3-pyridyl)-7-fluoro-6-[1-[3-(triazol-1-yl)propanoyl]-3,6-dihydro-2H-pyridin-5-yl]-1H-indole-2-carboxylate

A mixture of methyl 7-fluoro-4-(4,4,5,5-tetramethyl-1,3,2-dioxaborolan-2-yl)-6-[1-[3-(triazol-1-yl)propanoyl]-3,6-dihydro-2H-pyridin-5-yl]-1H-indole-2-carboxylate (400 mg, 764 μmol, Intermediate FM), 5-chloro-4-ethyl-2-methyl-pyridine (119 mg, 764 μmol, Intermediate PD), XPhos Pd G₃ (65 mg, 76 μmol), and K₂CO₃ (317 mg, 2.29 mmol) in dioxane (5 mL) and H₂O (1 mL) was degassed and purged with N₂ three times. Then the mixture was stirred at 90° C. for 12 hrs under N₂ atmosphere. Upon completion, the mixture was diluted with H₂O (20 mL) and extracted with EtOAc (10 mL×2). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 0˜100% EtOAc/PE gradient @80 mL/min) to give the title compound (300 mg, 61% yield) as a brown gum. LC-MS (ESI⁺) m/z 517.3 (M+1)⁺.

### Step 2—4-(4-Ethyl-6-methyl-3-pyridyl)-7-fluoro-6-[1-[3-(triazol-1-yl)propanoyl]-3,6-dihydro-2H-pyridin-5-yl]-1H-indole-2-carboxylic acid

To a solution of methyl 4-(4-ethyl-6-methyl-3-pyridyl)-7-fluoro-6-[1-[3-(triazol-1-yl)propanoyl]-3,6-dihydro-2H-pyridin-5-yl]-1H-indole-2-carboxylate (300 mg, 581 μmol) in THF (2 mL) and H₂O (2 mL) was added LiOH H₂O (122 mg, 2.90 mmol). The mixture was then stirred at 20° C. for 12 hrs. Upon completion, the mixture was acidified with HCl (2M) to adjust the pH=5. During this period, brown precipitate was formed, which was collected and dried under reduced pressure to give the title compound (300 mg) as a brown solid. LC-MS (ESI⁺) m/z 503.2 (M+1); ¹H NMR (400 MHz, DMSO-d₆) δ=12.83-12.48 (m, 1H), 8.66 (d, J=10.6 Hz, 1H), 8.16-8.07 (m, 1H), 8.04-7.94 (m, 1H), 7.74-7.64 (m, 1H), 7.14-7.03 (m, 1H), 6.81 (br s, 1H), 6.25-6.13 (m, 1H), 4.68-4.59 (m, 2H), 4.40-4.30 (m, 2H), 3.60-3.58 (m, 2H), 3.12-3.04 (m, 2H), 2.79 (s, 3H), 2.68-2.60 (m, 2H), 2.38-2.26 (m, 2H), 1.05 (t, J=7.6 Hz, 3H).

### 3-Methoxy-5-methyl-2-piperazin-1-yl-pyrazine (Intermediate PJ)

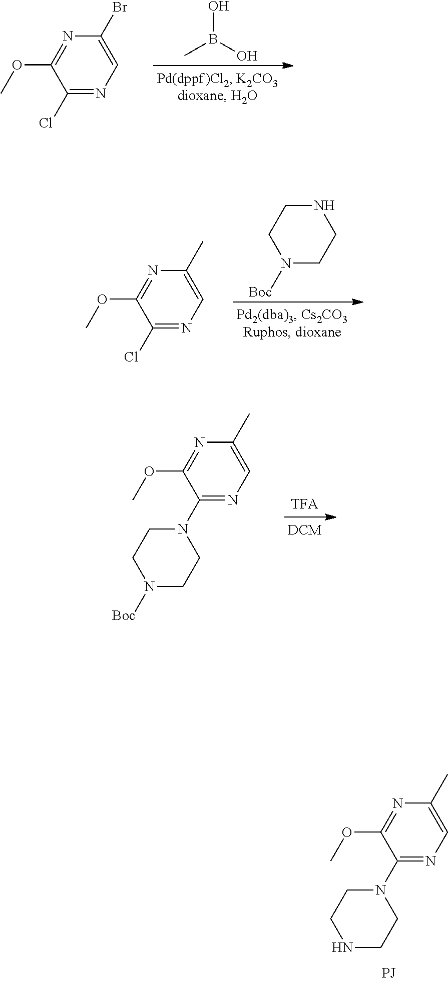

### Step 1—2-Chloro-3-methoxy-5-methyl-pyrazine

A mixture of 5-bromo-2-chloro-3-methoxy-pyrazine (1 g, 5 mmol, CAS #89283-94-3), MeB(OH)₂ (268 mg, 4.48 mmol), Pd(dppf)Cl₂ (327 mg, 448 μmol), and K₂CO₃ (1.86 g, 13.4 mmol) in dioxane (5 mL) and H₂O (1 mL) was degassed and purged with N₂ three times. Then the mixture was stirred at 80° C. for 12 h under N₂ atmosphere. On completion, the mixture was extracted with EtOAc (150 mL). The combined organic extracts were washed with saturated brine (30 mL) and then dried over Na₂SO₄, filtered and concentrated under vacuum. The residue was purified by column chromatography (SiO₂, PE/EtOAc=50/1 to 4/1) to give the title compound (350 mg, 49% yield) as a yellow solid. LC-MS (ESI⁺) m/z 159.0 (M+H)⁺.

### Step 2—Tert-butyl 4-(3-methoxy-5-methyl-pyrazin-2-yl)piperazine-1-carboxylate

A mixture of 2-chloro-3-methoxy-5-methyl-pyrazine (350 mg, 2.21 mmol), tert-butyl piperazine-1-carboxylate (493 mg, 2.65 mmol), Cs₂CO₃ (2.16 g, 6.62 mmol), RuPhos (103 mg, 221 μmol) and Pd₂(dba)₃ (202 mg, 221 μmol) in dioxane (10 mL) was degassed and purged with N₂ three times. Then the mixture was stirred at 100° C. for 12 h under N₂ atmosphere. On completion, the mixture was extracted with EtOAc (150 mL). The combined organic extracts were washed with saturated brine (30 mL) and then dried over Na₂SO₄, filtered and concentrated under vacuum. The residue was purified by prep-TLC (SiO₂, PE/EtOAc=3/1) to give title compound (460 mg, 59% yield) as a brown solid. LC-MS (ESI⁺) m/z 309.4 (M+H)⁺; ¹H NMR (400 MHz, DMSO-d₆) δ=7.63 (s, 1H), 3.89 (s, 3H), 3.42 (d, J=4.8 Hz, 4H), 3.30-3.27 (m, 4H), 2.28 (s, 3H), 1.41 (s, 9H).

### Step 3—3-Methoxy-5-methyl-2-piperazin-1-yl-pyrazine

To a solution of tert-butyl 4-(3-methoxy-5-methyl-pyrazin-2-yl)piperazine-1-carboxylate (450 mg, 1.46 mmol) in DCM (2 mL) was added TFA (40.39 mmol, 3 mL). The mixture was stirred at 20° C. for 0.5 h. On completion, the reaction mixture was concentrated under vacuum to give the title compound (300 mg, 99% yield) as a yellow oil.

### Tert-butyl 5-(5-(4-(5-fluoro-3-methoxypyridin-2-yl)piperazine-1-carbonyl)-7-(2-methoxyphenyl)-1H-indol-2-yl)-3,6-dihydropyridine-1(2H)-carboxylate (Intermediate PK)

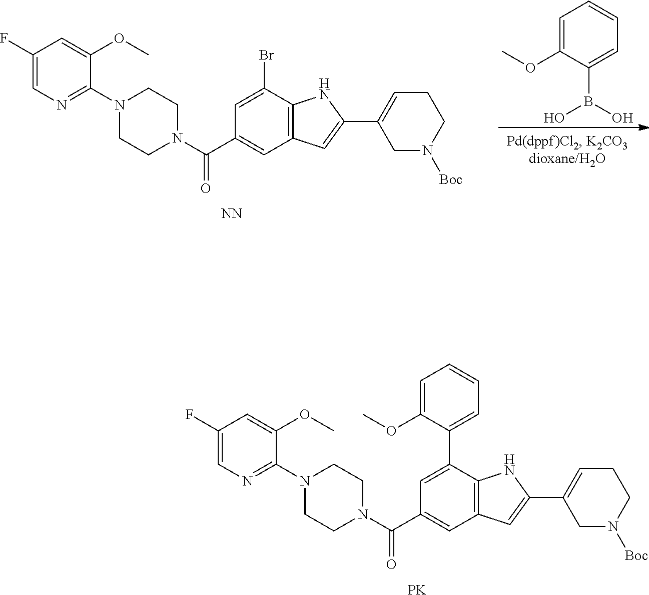

To a solution of tert-butyl 5-(7-bromo-5-(4-(5-fluoro-3-methoxypyridin-2-yl)piperazine-1-carbonyl)-1H-indol-2-yl)-3,6-dihydropyridine-1(2H)-carboxylate (500 mg, 813 μmol, Intermediate NN), (2-methoxyphenyl)boronic acid (185 mg, 1.22 mmol, CAS #5720-06-9) in dioxane (1 mL) and H₂O (0.3 mL) was added K₂CO₃ (337 mg, 2.44 mmol) and Pd(dppf)Cl₂ (59.5 mg, 81.3 μmol). Then the reaction was stirred at 80° C. for 1 hr under nitrogen atmosphere. On completion, the reaction mixture was filtered and the filtrate was concentrated in vacuo to give the crude residue. The crude residue was purified by column chromatography (SiO₂, Petroleum ether. Ethyl acetate=1/0 to 1/1) to give the title compound (400 mg, 74% yield) as a yellow solid. LC-MS (ESI⁺) m/z 642.2 (M+H)⁺.

### (4-(5-Fluoro-3-methoxypyridin-2-yl)piperazin-1-yl)(1-isobutyl-7-(2-methoxyphenyl)-2-(1,2,5,6-tetrahydropyridin-3-yl)-1H-indol-5-yl)methanone (Intermediate PL)

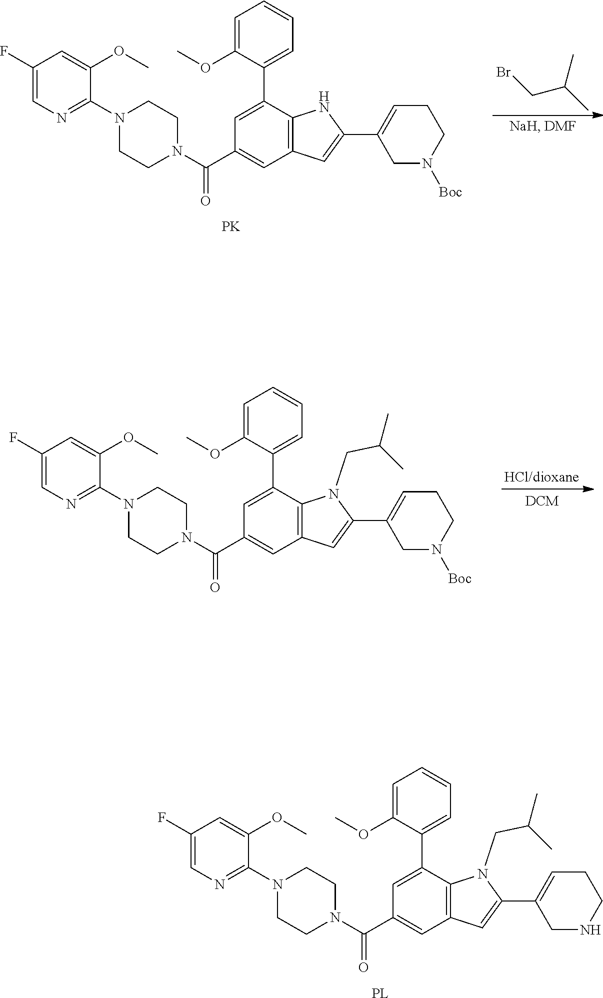

### Step 1—Tert-butyl 5-(5-(4-(5-fluoro-3-methoxypyridin-2-yl)piperazine-1-carbonyl)-1-isobutyl-7-(2-methoxyphenyl)-1H-indol-2-yl)-3,6-dihydropyridine-1(2H)-carboxylate

To a solution of tert-butyl 5-(5-(4-(5-fluoro-3-methoxypyridin-2-yl)piperazine-1-carbonyl)-7-(2-methoxyphenyl)-1H-indol-2-yl)-3,6-dihydropyridine-1(2H)-carboxylate (200 mg, 311 μmol, Intermediate PK) in DMF (2 mL) was added NaH (18.7 mg, 467 μmol, 60% dispersion in mineral oil) under N₂ atmosphere at 0° C. The mixture was stirred at 0° C. for 0.5 hrs, and then 1-bromo-2-methylpropane (64.5 mg, 467 μmol, 50.8 μL, CAS #78-77-3) was added at 0° C. The resulting mixture was then stirred at 25° C. for 12 hrs. On completion, the reaction mixture was quenched with sat. NH₄Cl (10 mL) and extracted with ethyl acetate (3×50 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 1/1) to give the title compound (160 mg, 55% yield) as a yellow solid. LC-MS (ESI⁺) m/z 698.4 (M+H)⁺; ¹H NMR (400 MHz, DMSO-d6) δ=7.78 (d, J=2.4 Hz, 1H), 7.67-7.57 (m, 1H), 7.49-7.43 (m, 1H), 7.37-7.32 (m, 2H), 7.18-7.13 (m, 1H), 7.08 (t, J=7.6 Hz, 1H), 6.94 (d, J=1.6 Hz, 1H), 6.55 (s, 1H), 5.97 (s, 1H), 4.26-4.14 (m, 1H), 4.05-3.94 (m, 1H), 3.83 (s, 3H), 3.80-3.74 (m, 1H), 3.72 (s, 3H), 3.68 (s, 4H), 3.60-3.50 (m, 2H), 3.45 (dd, J=6.4, 14.4 Hz, 2H), 3.24 (s, 4H), 2.28 (s, 2H), 1.41 (s, 9H), 0.32-0.07 (m, 6H).

### Step 2—(4-(5-Fluoro-3-methoxypyridin-2-yl)piperazin-1-yl)(1-isobutyl-7-(2-methoxyphenyl)-2-(1,2,5,6-tetrahydropyridin-3-yl)-1H-indol-5-yl)methanone

To a solution of tert-butyl 5-(5-(4-(5-fluoro-3-methoxypyridin-2-yl)piperazine-1-carbonyl)-1-isobutyl-7-(2-methoxyphenyl)-1H-indol-2-yl)-3,6-dihydropyridine-1(2H)-carboxylate (160 mg, 229 μmol) in DCM (2 mL) was added HCl/dioxane (8 M, 1 mL). The mixture was stirred at 25° C. for 1 hr. On completion, the reaction mixture was concentrated in vacuo to give the title compound (140 mg, HCl) as a yellow solid. LC-MS (ESI⁺) m/z 598.5 (M+H)⁺.

### Methyl 6-(1-tert-butoxycarbonyl-3,6-dihydro-2H-pyridin-5-yl)-7-fluoro-4-(4,4,5,5-tetramethyl-1,3,2-dioxaborolan-2-yl)-1H-indole-2-carboxylate (Intermediate PM)

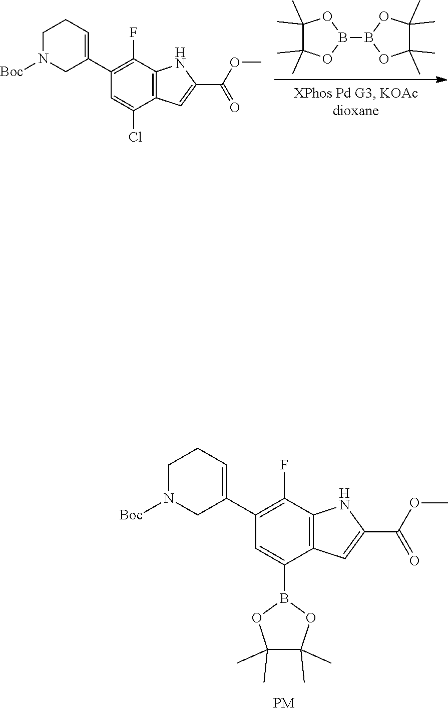

To a solution of methyl 6-(1-(tert-butoxycarbonyl)-1,2,5,6-tetrahydropyridin-3-yl)-4-chloro-7-fluoro-1H-indole-2-carboxylate (5.90 g, 14.4 mmol, synthesized via Step 1 of Intermediate AO) and 4,4,5,5-tetramethyl-2-(4,4,5,5-tetramethyl-1,3,2-dioxaborolan-2-yl)-1,3,2-dioxaborolane (36.65 g, 144.3 mmol, CAS #73183-34-3) in dioxane (500 mL) were added KOAc (4.25 g, 43.29 mmol) and XPhos Pd G₃ (1.22 g, 1.44 mmol). The mixture was then stirred at 80° C. for 5 h. Upon completion, the mixture was diluted with H₂O (20 mL) and extracted with EtOAc (25 mL×2). The combined organic layers were dried over Na₂SO₄, filtered and concentrated under reduced pressure to give a residue. The residue was purified by flash silica gel chromatography (ISCO®; 40 g SepaFlash® Silica Flash Column, Eluent of 0 100% EtOAc/Petroleum ether gradient @60 mL/min) to give the title compound (3.70 g, 49% yield) as a white solid. LC-MS (ESI⁺) m/z 445.1 (M−56)⁺; ¹H NMR (400 MHz, CDCl₃-d) δ=9.02 (s, 1H), 7.71-7.61 (m, 1H), 7.56 (d, J=7.2 Hz, 1H), 6.11 (s, 1H), 4.28 (s, 2H), 3.98 (s, 3H), 3.65-3.55 (m, 2H), 2.35 (s, 2H), 1.43-1.36 (m, 12H), 1.28 (s, 9H).

### Methyl 7-fluoro-6-(1,2,3,6-tetrahydropyridin-5-yl)-4-(4,4,5,5-tetramethyl-1,3,2-dioxaborolan-2-yl)-1H-indole-2-carboxylate (Intermediate PN)

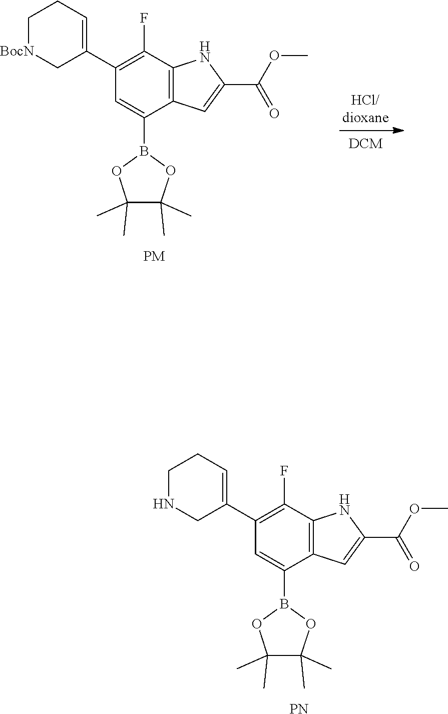

To a solution of methyl 6-(1-tert-butoxycarbonyl-3,6-dihydro-2H-pyridin-5-yl)-7-fluoro-4-(4,4,5,5-tetramethyl-1,3,2-dioxaborolan-2-yl)-1H-indole-2-carboxylate (600 mg, 1.20 mmol, Intermediate PM) in DCM (2 mL) was added HCl/dioxane (2 M, 599.56 μL). The mixture was then stirred at 20° C. for 2 hr. On completion, the reaction mixture was concentrated under vacuum to give title compound (520 mg, HCl) as a yellow solid.

### Methyl 7-fluoro-4-(4,4,5,5-tetramethyl-1,3,2-dioxaborolan-2-yl)-6-(1-(3-(thiazol-2-yl)propanoyl)-1,2,5,6-tetrahydropyridin-3-yl)-1H-indole-2-carboxylate (Intermediate PO)

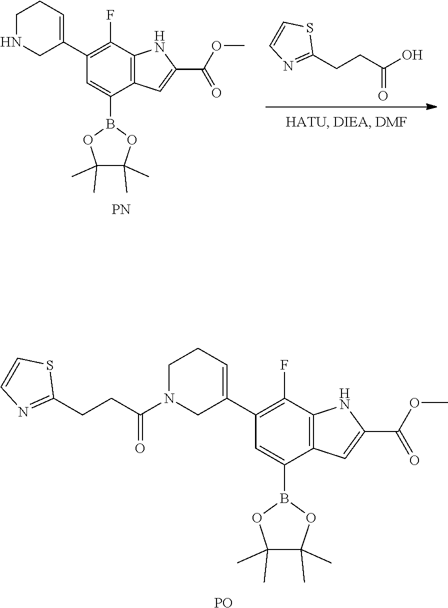

To a solution of methyl 7-fluoro-6-(1,2,3,6-tetrahydropyridin-5-yl)-4-(4,4,5,5-tetramethyl-1,3,2-dioxaborolan-2-yl)-1H-indole-2-carboxylate (520 mg, 1.19 mmol, HCl, Intermediate PN) in DMF (10 mL) was added 3-(thiazol-2-yl)propanoic acid (187 mg, 1.19 mmol, CAS #144163-65-5) and DIEA (462 mg, 3.57 mmol). Then HATU (543 mg, 1.43 mmol) was added and the mixture was stirred at 20° C. for 12 h. On completion, the mixture was extracted with EtOAc (60 mL). The combined organic extracts were washed with saturated brine (20 mL) and then dried over Na₂SO₄, filtered and concentrated under vacuum. The residue was purified by column chromatography (SiO₂, Petroleum ether/Ethyl acetate=10/1 to 0/1) to give the title compound (800 mg, 1.19 mmol, quant. yield) as a yellow solid. LC-MS (ESI⁺) m/z 540.2 (M+H)⁺.

### 4-(4-Ethyl-6-methyl-3-pyridyl)-7-fluoro-6-[1-(3-thiazol-2-ylpropanoyl)-3,6-dihydro-2H-pyridin-5-yl]-1H-indole-2-carboxylic acid (Intermediate PP)

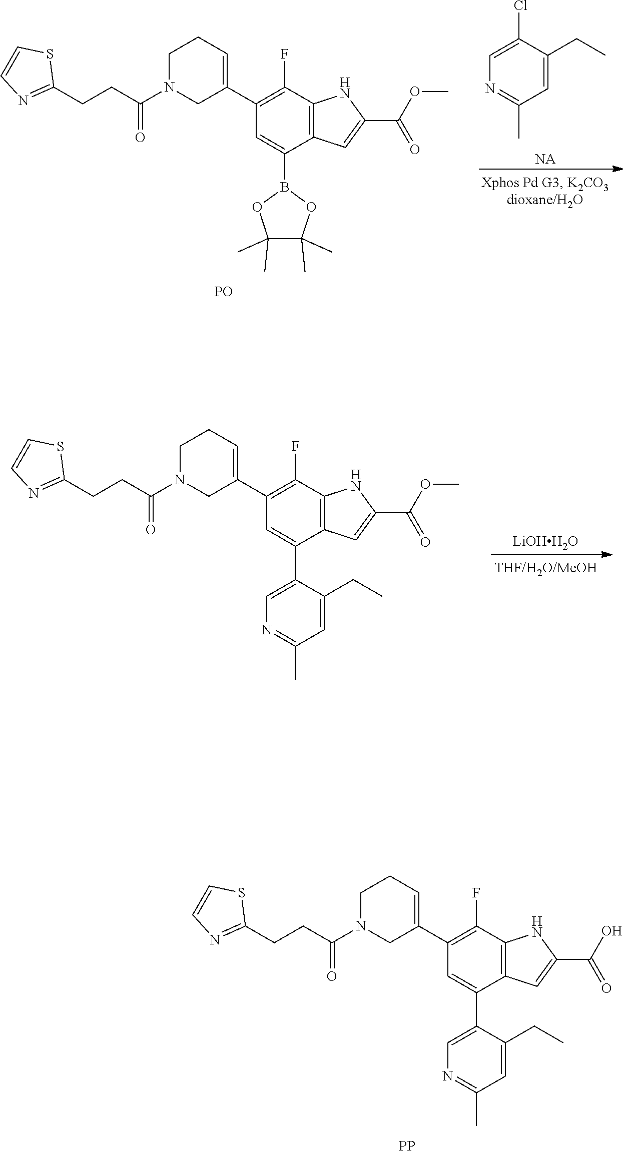

### Step 1—Methyl 4-(4-ethyl-6-methylpyridin-3-yl)-7-fluoro-6-(1-(3-(thiazol-2-yl)propanoyl)-1,2,5,6-tetrahydropyridin-3-yl)-1H-indole-2-carboxylate

A mixture of methyl 7-fluoro-4-(4,4,5,5-tetramethyl-1,3,2-dioxaborolan-2-yl)-6-[1-(3-thiazol-2-ylpropanoyl)-3,6-dihydro-2H-pyridin-5-yl]-1H-indole-2-carboxylate (700 mg, 1.30 mmol, Intermediate PO), 5-chloro-4-ethyl-2-methylpyridine (202 mg, 1.30 mmol, Intermediate NA), XPhos Pd G3 (110 mg, 129.77 μmol), and K₂CO₃ (538 mg, 3.89 mmol) in dioxane (10 mL) and H₂O (2 mL) was degassed and purged with N₂ three times. Then the mixture was stirred at 80° C. for 12 h under N₂ atmosphere. On completion, the mixture was extracted with EtOAc (90 mL). The combined organic extracts were washed with saturated brine (20 mL) and then dried over Na₂SO₄, filtered and concentrated under vacuum. The residue was purified by column chromatography (SiO₂, DCM/MeOH=50/1 to 20/1) to give the title compound (460 mg, 67% yield) as a brown solid. LC-MS (ESI⁺) m/z 533.2 (M+H)⁺.

### Step 2—4-(4-Ethyl-6-methyl-3-pyridyl)-7-fluoro-6-[1-(3-thiazol-2-ylpropanoyl)-3,6-dihydro-2H-pyridin-5-yl]-1H-indole-2-carboxylic acid

To a solution of methyl 4-(4-ethyl-6-methyl-3-pyridyl)-7-fluoro-6-[1-(3-thiazol-2-ylpropanoyl)-3,6-dihydro-2H-pyridin-5-yl]-1H-indole-2-carboxylate (460 mg, 863 μmol) in MeOH (2 mL), H₂O (1 mL) and THF (5 mL) was added LiOH·H₂O (181 mg, 4.32 mmol). The mixture was then stirred at 20° C. for 12 h. On completion, the reaction mixture was quenched with HCl (10 mL 2 M), and then extracted with DCM (150 mL). The combined organic layers were dried over Na₂SO₄, filtered and concentrated under reduced pressure to give the title compound (430 mg, 96% yield) as a brown solid. LC-MS (ESI⁺) m/z 519.2 (M+H)⁺.

### N-([1,2,4]triazolo[4,3-a]pyridin-3-ylmethyl)-5-(3-methoxy-4-(2-oxopyrrolidin-1-yl)phenyl)-N4-dimethylthiophene-2-carboxamide (Intermediate PR)

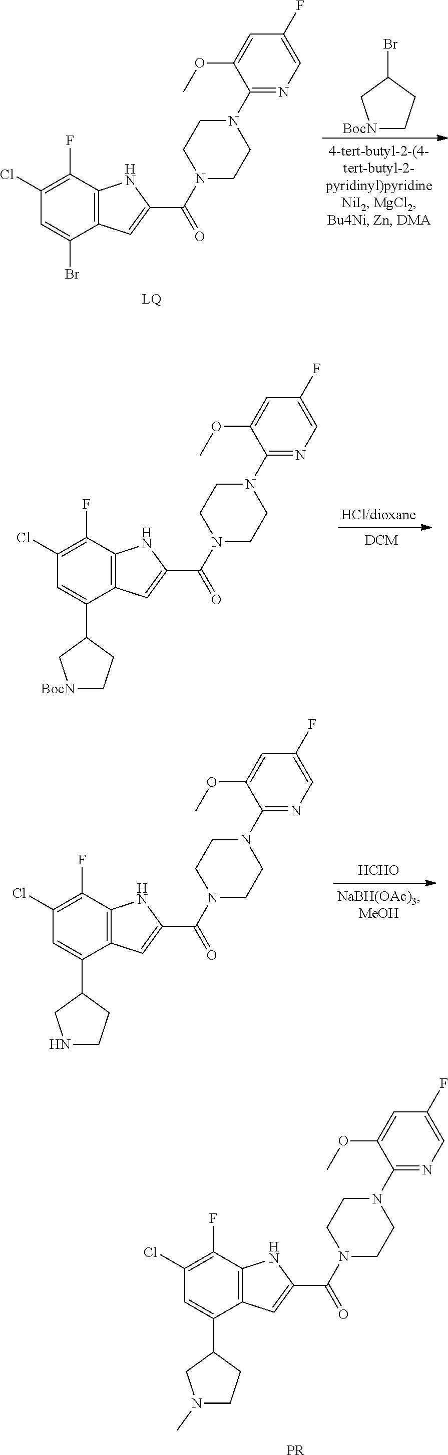

### Step 1—Tert-butyl 3-[6-chloro-7-fluoro-2-[4-(5-fluoro-3-methoxy-2-pyridyl)piperazine-1-carbonyl]-1H-indol-4-yl]pyrrolidine-1-carboxylate

To a solution of (4-bromo-6-chloro-7-fluoro-1H-indol-2-yl)-[4-(5-fluoro-3-methoxy-2-pyridyl)piperazin-1-yl]methanone (400 mg, 824 μmol, Intermediate LQ) in DMA (2.00 mL)) was added tert-butyl 3-bromopyrrolidine-1-carboxylate (308.99 mg, 1.24 mmol, CAS #939793-16-5), diiodonickel (25.74 mg, 82.35 μmol), dichloromagnesium (78.41 mg, 823.5 μmol), tetrabutylammonium iodide (304.19 mg, 823.54 μmol), 4-tert-butyl-2-(4-tert-butyl-2-pyridyl)pyridine (22.10 mg, 82.35 μmol), and Zinc (215.40 mg, 3.29 mmol). The reaction was then stirred at 80° C. for 12 hr under N₂ atmosphere. Upon completion, the mixture was filtered and concentrated under reduced pressure to give a residue. The crude product was purified by reversed-phase HPLC (0.1% FA condition) to give the title compound (180 mg, 21% yield) as a white solid. LC-MS (ESI⁺) m/z 576.3 (M+H)⁺; ¹H NMR (400 MHz, DMSO-d6) δ (ppm)=12.57-11.94 (m, 1H), 7.80 (d, J=2.4 Hz, 1H), 7.42-7.35 (m, 1H), 7.07 (s, 1H), 7.04-6.98 (m, 1H), 3.86 (s, 3H), 3.84 (s, 2H), 3.82 (br s, 2H), 3.78-3.71 (m, 2H), 3.48 (d, J=5.6 Hz, 2H), 3.40-3.36 (m, 2H), 3.36-3.34 (m, 2H), 3.21-3.08 (m, 1H), 2.25-2.06 (m, 2H), 1.41 (m, 9H).

### Step 2—(6-Chloro-7-fluoro-4-(pyrrolidin-3-yl)-1H-indol-2-yl)(4-(5-fluoro-3-methoxypyridin-2-yl)piperazin-1-yl)methanone

To a solution of tert-butyl 3-[6-chloro-7-fluoro-2-[4-(5-fluoro-3-methoxy-2-pyridyl)piperazine-1-carbonyl]-1H-indol-4-yl]pyrrolidine-1-carboxylate (300 mg, 521 μmol) in HCl/dioxane (3 mL) was stirred at 25° C. for 0.5 hr. Upon completion, the mixture was concentrated under reduced pressure to give the title compound (300 mg) as a yellow solid. LC-MS (ESI⁺) m/z 476.1 (M+H)⁺.

### Step 3—N-([1,2,4]triazolo[4,3-a]pyridin-3-ylmethyl)-5-(3-methoxy-4-(2-oxopyrrolidin-1-yl)phenyl)-N,4-dimethylthiophene-2-carboxamide

To a solution of (6-chloro-7-fluoro-4-pyrrolidin-3-yl-1H-indol-2-yl)-[4-(5-fluoro-3-methoxy-2-pyridyl)piperazin-1-yl]methanone (300 mg, 630 μmol) in THF (2.00 mL) and DMSO (2.00 mL) was added HCHO (358.08 mg, 4.41 mmol) and NaOAc (155.13 mg, 1.89 mmol). The mixture was then stirred at 25° C. for 30 min. Then added NaBH(OAc)₃ (400.80 mg, 1.89 mmol) was stirred at 0° C. for 6 min. Then the mixture was stirred at 25° C. for 1.5 hr. Upon completion, the mixture was filtered to obtain the crude reaction solution. The crude product was purified by reversed-phase HPLC(Neutral) to give the title compound (102 mg, 31% yield) as a white solid. LC-MS (ESI⁺) m/z 490.2 (M+H)⁺.

### Tert-butyl 3-(6-chloro-7-fluoro-2-(4-(5-fluoro-3-methoxypyridin-2-yl)piperazine-1-carbonyl)-1H-indol-4-yl)azetidine-1-carboxylate (Intermediate PS)

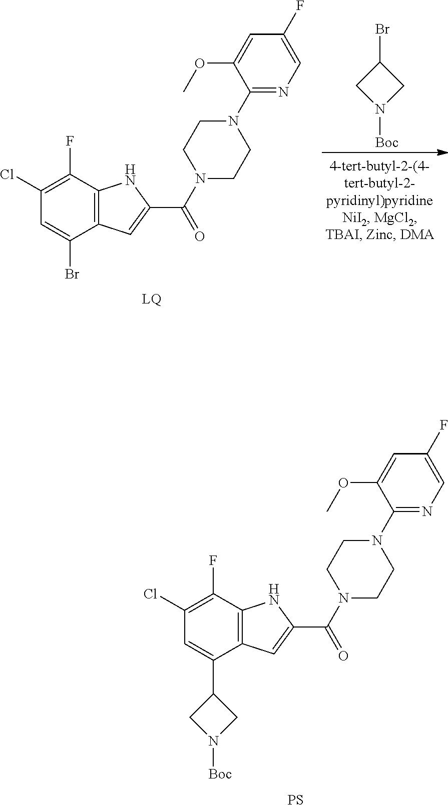 [SMILES: COc1cc(F)cnc1N1CCN(C(=O)c2cc3c(Br)cc(Cl)c(F)c3[nH]2)CC1 ; CC(C)(C)OC(=O)N1CC(Br)C1 ; COc1cc(F)cnc1N1CCN(C(=O)c2cc3c(C4CN(C(=O)OC(C)(C)C)C4)cc(Cl)c(F)c3[nH]2)CC1 ; "PS"]

To a solution of (4-bromo-6-chloro-7-fluoro-1H-indol-2-yl)-[4-(5-fluoro-3-methoxy-2-pyridyl)piperazin-1-yl]methanone (500.00 mg, 1.03 mmol, Intermediate LQ), tert-butyl 3-bromoazetidine-1-carboxylate (364.58 mg, 1.54 mmol), diiodonickel (64.34 mg, 205.9 μmol, 11.04 μL), dichloromagnesium (98.01 mg, 1.03 mmol, 42.25 μL), tetrabutylammonium iodide (380.24 mg, 1.03 mmol), 4-tert-butyl-2-(4-tert-butyl-2-pyridyl)pyridine (27.63 mg, 102.9 μmol) in DMA (5 mL) was added Zinc (201.94 mg, 3.09 mmol) under N₂. The reaction was then stirred at 60° C. for 5 h under N₂ atmosphere. Upon completion, the mixture was filtered and the filtrate was concentrated under reduced pressure to give a mixture. The mixture was diluted with H₂O (10 mL) and extracted with EtOAc (10 mL×2) and was concentrated under reduced pressure to give a residue. The residue was purified by flash silica gel chromatography (ISCO®; 20 g SepaFlash® Silica Flash Column, Eluent of 0˜70% EtOAc/Petroleum ether gradient @80 mL/min) to give the title compound (120.00 mg, 15% yield) as a light yellow solid. ¹H NMR (400 MHz, DMSO-d₆) δ=12.52-12.07 (m, 1H), 7.80 (d, J=2.4 Hz, 1H), 7.38 (dd, J=2.4, 10.8 Hz, 1H), 7.12 (d, J=6.0 Hz, 1H), 6.89-6.79 (m, 1H), 4.32 (d, J=7.2 Hz, 2H), 4.21-4.05 (m, 2H), 4.04-3.93 (m, 2H), 3.86 (s, 3H), 3.82 (s, 4H), 3.17 (d, J=5.2 Hz, 3H), 1.42-1.32 (m, 9H).

### 6-(1-(Tert-butoxycarbonyl)-1,2,5,6-tetrahydropyridin-3-yl)-7-fluoro-4-(4,4,5,5-tetramethyl-1,3,2-dioxaborolan-2-yl)-1H-indole-2-carboxylic acid (Intermediate PT)

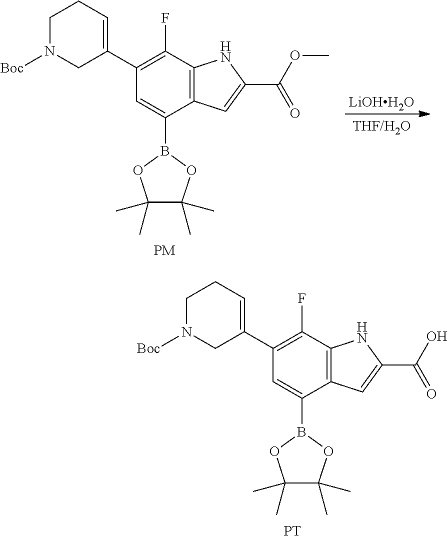

To a solution of methyl 6-(1-tert-butoxycarbonyl-3,6-dihydro-2H-pyridin-5-yl)-7-fluoro-4-(4,4,5,5-tetramethyl-1,3,2-dioxaborolan-2-yl)-1H-indole-2-carboxylate (0.50 g, 999.27 μmol, Intermediate PM) in THF (3 mL) and H₂O (3 mL) was added LiOH·H₂O (209.66 mg, 5.00 mmol). The mixture was then stirred at 20° C. for 0.5 h. Upon completion, the mixture was adjusted to pH=5 with 2M HCl. The residue was then extracted with EtOAc (2 mL×3). The combined organic layers were dried over Na₂SO₄, filtered and concentrated under reduced pressure to give the title compound (480.00 mg, 89% yield) as a white solid. LC-MS (ESI+) m/z 387.2 (M−99)⁺; ¹H NMR (400 MHz, DMSO-d₆) δ=12.32 (s, 1H), 7.46-7.28 (m, 2H), 6.04 (s, 1H), 4.30-4.09 (m, 2H), 3.50 (t, J=5.6 Hz, 2H), 2.28 (s, 2H), 1.54-1.27 (m, 21H).

### Tert-butyl 3-(7-fluoro-2-(4-(5-fluoro-3-methoxypyridin-2-yl)piperazine-1-carbonyl)-4-(4,4,5,5-tetramethyl-1,3,2-dioxaborolan-2-yl)-1H-indol-6-yl)-5,6-dihydropyridine-1(2H)-carboxylate (Intermediate PU)

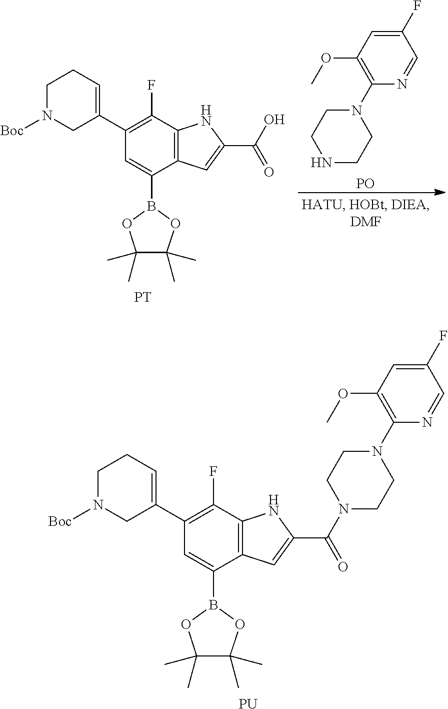

To a solution of 6-(1-(tert-butoxycarbonyl)-1,2,5,6-tetrahydropyridin-3-yl)-7-fluoro-4-(4,4,5,5-tetramethyl-1,3,2-dioxaborolan-2-yl)-1H-indole-2-carboxylic acid (480.00 mg, 986.96 μmol, Intermediate PT) and 1-(5-fluoro-3-methoxy-2-pyridyl)piperazine (250.18 mg, 1.18 mmol, Intermediate BO), DIEA (765.35 mg, 5.92 mmol, 1.03 mL), and HOBt (266.72 mg, 1.97 mmol) in DMF (6 mL) was added HATU (450.33 mg, 1.18 mmol). Then the mixture was stirred at 25° C. for 0.5 h. Upon completion, the mixture was filtered and the filter cake was dried to give the title compound (500.00 mg, 69% yield) as a white solid. LC-MS (ESI⁺) m/z 680.4 (M+H)⁺; ¹H NMR (400 MHz, DMSO-d₆) δ=12.14 (s, 1H), 7.95 (s, 1H), 7.80 (d, J=2.4 Hz, 1H), 7.43-7.31 (m, 2H), 6.98 (s, 1H), 6.03 (s, 1H), 4.18 (s, 2H), 3.85 (s, 3H), 3.78 (s, 4H), 3.51 (t, J=5.6 Hz, 2H), 3.30-3.26 (m, 4H), 2.28 (d, J=2.8 Hz, 2H), 1.43 (s, 9H), 1.33 (s, 11H).

### Succinic acid (CAS #110-15-6) (Intermediate PV)

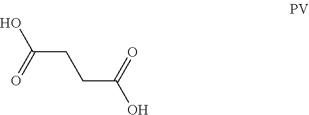

### 2-Ethyl-6-methyl-3-(4,4,5,5-tetramethyl-1,3,2-dioxaborolan-2-yl)pyridine (Intermediate PW)

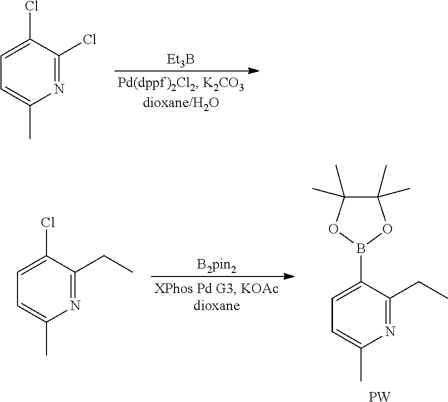

### Step 1—3-Chloro-2-ethyl-6-methylpyridine

To a solution of 2,3-dichloro-6-methylpyridine (10 g, 62 mmol, CAS #54957-86-7) and triethylborane (1 M, 61 mL) in dioxane (120 mL) and H₂O (24 mL) was added K₂CO₃ (25.5 g, 185 mmol) and Pd(dppf)Cl₂·CH₂Cl₂ (5.04 g, 6.17 mmol) at 25° C. under nitrogen atmosphere. Then the mixture was stirred at 65° C. for 12 hrs under nitrogen atmosphere. On completion, the reaction mixture was quenched with water (100 mL) and extracted with ethyl acetate (3×100 mL). The combined organic layers were washed with brine (200 mL) and dried over anhydrous sodium sulfate, filtered and the filtrate was concentrated in vacuo to give the crude residue. The residue was purified by column chromatography (SiO₂, Petroleum ether/Ethyl acetate=20/1 to 10/1) to give the title compound (6.6 g, 69% yield) as a yellow oil. LC-MS (ESI⁺) m/z 156.1 (M+H)⁺; ¹H NMR (400 MHz, DMSO-d₆) δ=7.70 (d, J=8.4 Hz, 1H), 7.14-7.09 (m, 1H), 2.82 (q, J=7.2 Hz, 2H), 2.43 (s, 3H), 1.19 (t, J=7.6 Hz, 3H).

### Step 2—2-Ethyl-6-methyl-3-(4,4,5,5-tetramethyl-1,3,2-dioxaborolan-2-yl)pyridine

To a solution of 4,4,5,5-tetramethyl-2-(4,4,5,5-tetramethyl-1,3,2-dioxaborolan-2-yl)-1,3,2-dioxaborolane (7.34 g, 28.9 mmol) and 3-chloro-2-ethyl-6-methylpyridine (3 g, 20 mmol) in dioxane (30 mL) was added XPhos Pd G3 (1.63 g, 1.93 mmol) and AcOK (5.68 g, 57.8 mmol) at 25° C. under nitrogen atmosphere. Then the mixture was stirred at 80° C. for 2 hrs under nitrogen atmosphere. On completion, the reaction mixture was concentrated in vacuo to give the crude residue. The residue was purified by column chromatography (SiO₂, Petroleum ether/Ethyl acetate=10/1 to 0/1) to give the title compound (1 g, 21% yield) as a white solid. LC-MS (ESI⁺) m/z 248.1 (M+H)⁺; ¹H NMR (400 MHz, DMSO-d₆) δ=7.83 (d, J=7.6 Hz, 1H), 7.05 (d, J=7.6 Hz, 1H), 2.93 (q, J=7.6 Hz, 2H), 2.44 (s, 2H), 1.17-1.14 (m, 12H), 1.08 (s, 3H).

### Methyl 2-amino-4-bromobenzo[d]thiazole-6-carboxylate (Intermediate PX)

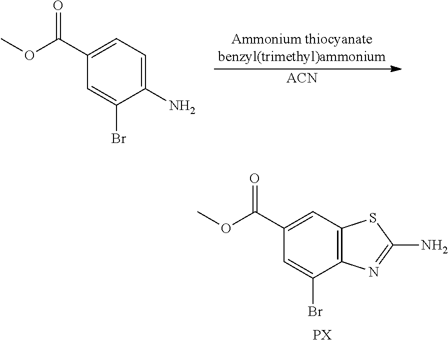

To a solution of methyl 4-amino-3-bromobenzoate (10 g, 43 mmol, CAS #106896-49-5) in anhydrous ACN (180 mL) was added ammonia thiocyanic acid (4.96 g, 65.2 mmol, 4.96 mL) and benzyl(trimethyl)ammonium (16.9 g, 43.4 mmol) in ACN (45 mL) at 25° C. under nitrogen atmosphere. Then the reaction was stirred at 25° C. for 12 hrs under nitrogen atmosphere. On completion, the reaction mixture was quenched withy water (50 mL) and extracted with ethyl acetate (3×50 mL). The combined organic layers were washed with brine (50 mL) and dried over anhydrous sodium sulfate, filtered and the filtrate was concentrated in vacuo to give the crude residue. The residue was purified by column chromatography (SiO₂, Petroleum ether/Ethyl acetate=5/1 to 3/1) to give the title compound (7 g, 34% yield,) as a white solid. LC-MS (ESI⁺) m/z 288.9 (M+H)⁺.

### Methyl 2-bromo-4-(2-ethyl-6-methylpyridin-3-yl)benzo[d]thiazole-6-carboxylate (Intermediate PY)

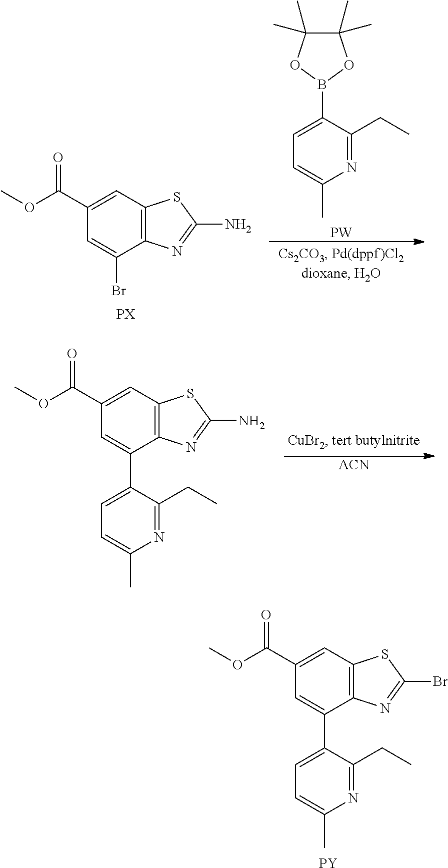

### Step 1—Methyl 2-amino-4-(2-ethyl-6-methylpyridin-3-yl)benzo[d]thiazole-6-carboxylate

To a solution of 2-ethyl-6-methyl-3-(4,4,5,5-tetramethyl-1,3,2-dioxaborolan-2-yl)pyridine (826 mg, 3.34 mmol, Intermediate PW) and methyl 2-amino-4-bromobenzo[d]thiazole-6-carboxylate (800 mg, 1.67 mmol, Intermediate PX) in dioxane (10 mL) and H₂O (2 mL) was added Pd(dppf)Cl₂·CH₂Cl₂ (136 mg, 167 μmol) and Cs₂CO₃ (1.63 g, 5.02 mmol) at 25° C. under nitrogen atmosphere. Then the mixture was stirred at 100° C. for 12 hrs under nitrogen atmosphere. On completion, the reaction mixture was concentrated in vacuo to give the crude residue. The crude residue was purified by column chromatography (SiO₂, Petroleum ether/Ethyl acetate=5/1 to 0/1) to give the title compound (400 mg, 53% yield) as a yellow solid. LC-MS (ESI⁺) m/z 328.1 (M+H)⁺.

### Step 2—Methyl 2-bromo-4-(2-ethyl-6-methylpyridin-3-yl)benzo[d]thiazole-6-carboxylate

To a solution of methyl 2-amino-4-(2-ethyl-6-methylpyridin-3-yl)benzo[d]thiazole-6-carboxylate (400 mg, 1.22 mmol) in anhydrous ACN (4 mL) was added t-BuONO (151 mg, 1.47 mmol, 174 μL) and CuBr₂ (409 mg, 1.83 mmol, 85.8 μL) at 0° C. Then the reaction was stirred at 25° C. for 2 hrs. On completion, the reaction mixture was concentrated in vacuo to give the crude residue. The residue was purified by column chromatography (SiO₂, Petroleum ether/Ethyl acetate=5/1 to 0/1) to give the title compound (400 mg, 64% yield) as a yellow solid. LC-MS (ESI⁺) m/z 392.0 (M+H)⁺.

### 2-(1-(3-(1H-1,2,3-triazol-1-yl)propanoyl)-1,2,5,6-tetrahydropyridin-3-yl)-4-(2-ethyl-6-methylpyridin-3-yl)benzo[d]thiazole-6-carboxylic acid (Intermediate PZ)

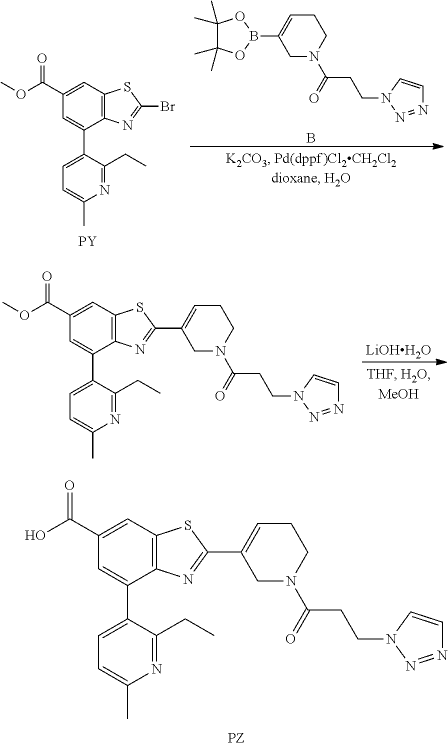

### Step 1—Methyl 2-(1-(3-(1H-1,2,3-triazol-1-yl)propanoyl)-1,2,5,6-tetrahydropyridin-3-yl)-4-(2-ethyl-6-methylpyridin-3-yl)benzo[d]thiazole-6-carboxylate

To a solution of 1-(5-(4,4,5,5-tetramethyl-1,3,2-dioxaborolan-2-yl)-3,6-dihydropyridin-1(2H)-yl)-3-(1H-1,2,3-triazol-1-yl)propan-1-one (339 mg, 1.02 mmol, Intermediate B) and methyl 2-bromo-4-(2-ethyl-6-methylpyridin-3-yl)benzo[d]thiazole-6-carboxylate (400 mg, 1.02 mmol, Intermediate PY) in dioxane (3 mL) and H₂O (0.6 mL) was added K₂CO₃ (423 mg, 3.07 mmol) and Pd(dppf)Cl₂·CH₂Cl₂ (166 mg, 204 μmol) at 25° C. under nitrogen atmosphere. Then the mixture was stirred at 80° C. for 2 hrs under nitrogen atmosphere. On completion, the reaction mixture was concentrated in vacuo to give the crude residue. The crude residue was purified by column chromatography (SiO₂, Petroleum ether/Ethyl acetate=10/1 to 0/1) to give the title compound (500 mg, 41% yield) as a yellow solid. LC-MS (ESI⁺) m/z 517.2 (M+H)⁺.

### Step 2—2-(1-(3-(1H-1,2,3-triazol-1-yl)propanoyl)-1,2,5,6-tetrahydropyridin-3-yl)-4-(2-ethyl-6-methylpyridin-3-yl)benzo[d]thiazole-6-carboxylic acid

To a solution of methyl 2-(1-(3-(1H-1,2,3-triazol-1-yl)propanoyl)-1,2,5,6-tetrahydropyridin-3-yl)-4-(2-ethyl-6-methylpyridin-3-yl)benzo[d]thiazole-6-carboxylate (500 mg, 416 μmol) in anhydrous THF (4 mL), MeOH (1 mL), and H₂O (1 mL) was added LiOH·H₂O (87.3 mg, 2.08 mmol) at 25° C., then the reaction was stirred at 25° C. for 1 hr. On completion, the reaction mixture was added HCl (1N) until the pH 4, then diluted with water (8 mL) and extracted with ethyl acetate (3×8 mL). The combined organic layers were dried over anhydrous sodium sulfate, filtered and the filtrate was concentrated in vacuo to give the title compound (200 mg) as a yellow solid. LC-MS (ESI⁺) m/z 503.6 (M+H)⁺.

### (2-Fluorophenyl)boronic acid (CAS #1993-03-9) (Intermediate QA)

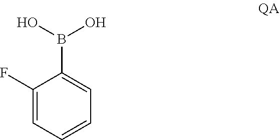

### (2,5-Difluorophenyl)boronic acid (CAS #193353-34-3) (Intermediate OB)

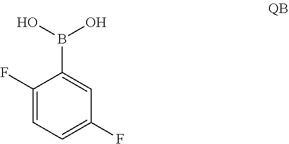 [SMILES: OB(O)c1cc(F)ccc1F]

### 5-bromo-1-methyl-pyrazole (CAS #361476-01-9) (Intermediate QC)

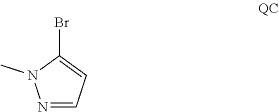

### 1-[5-[2-(1-Ethyl-4,6-dihydropyrrolo[3,4-c]pyrazole-5-carbonyl)-7-fluoro-4-(4,4,5,5-tetramethyl-1,3,2-dioxaborolan-2-yl)-1H-indol-6-yl]-3,6-dihydro-2H-pyridin-1-yl]-3-(triazol-1-yl)propan-1-one (Intermediate OD)

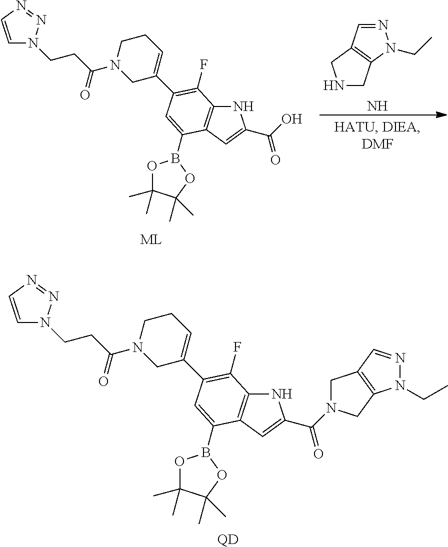

To a solution of 7-fluoro-4-(4,4,5,5-tetramethyl-1,3,2-dioxaborolan-2-yl)-6-[1-[3-(triazol-1-yl)propanoyl]-3,6-dihydro-2H-pyridin-5-yl]-1H-indole-2-carboxylic acid (700 mg, 1.37 mmol, Intermediate ML) in DMF (5 mL) was added 1-ethyl-5,6-dihydro-4H-pyrrolo[3,4-c]pyrazole (226 mg, 1.65 mmol, Intermediate NH) and DIEA (533 mg, 4.12 mmol). Then HATU (627 mg, 1.65 mmol) was added and the mixture was stirred at 20° C. for 2 h. On completion, the reaction mixture was diluted with EtOAc (30 mL) and filtered to give the title compound (800 mg, 93% yield) as a yellow solid. LC-MS (ESI⁺) m/z 629.2 (M+H)⁺.

### 5-Chloro-2-cyclopropyl-4-methyl-pyridine (Intermediate QE)

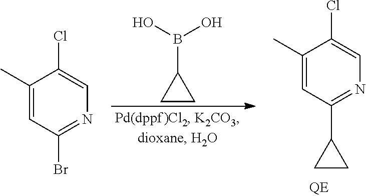

A mixture of 2-bromo-5-chloro-4-methyl-pyridine (1 g, 4.84 mmol, CAS #885267-40-3), cyclopropylboronic acid (1.66 g, 19.4 mmol), Pd(dppf)Cl₂ (354 mg, 484 μmol), K₂CO₃ (2.01 g, 14.5 mmol) in dioxane (10 mL) and H₂O (2 mL) was degassed and purged with N₂ three times. Then the mixture was stirred at 80° C. for 12 h under N₂ atmosphere. On completion, the reaction mixture was extracted with EtOAc (150 mL). The combined organic layers were dried over Na₂SO₄, filtered and concentrated under vacuum to give a residue. The residue was purified by prep-TLC (SiO₂, PE/EtOAc=10/1) to give the title compound (200 mg, 25% yield) as a yellow oil. LC-MS (ESI⁺) m/z 168.1 (M+H)⁺.

### 5-Chloro-2-ethyl-4-methylpyridine (Intermediate OF)

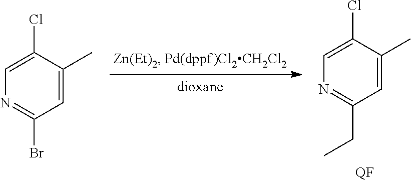

A mixture of 2-bromo-5-chloro-4-methylpyridine (5 g, 20 mmol), ZnEt₂ (1 M, 12.1 mL) and Pd(dppf)Cl₂·CH₂Cl₂ (1.98 g, 2.42 mmol) in dioxane (100 mL) was degassed and purged with N₂ three times. Then the mixture was stirred at 80° C. for 3 hrs under N₂ atmosphere. On completion, the reaction mixture was quenched with H₂O (5 mL) at 0° C., and then extracted with ethyl acetate (5 mL×2). The combined organic layers were washed with brine (15 mL), dried over anhydrous sodium sulfate, filtered and concentrated under reduced pressure to give a residue. The residue was purified by column chromatography (SiO₂, Petroleum ether:Ethyl acetate=0/1 to 10/1) to give the title compound (740 mg, 19% yield) as a yellow oil. LC-MS (ESI+) m/z 156.1 (M+H)⁺; ¹H NMR (400 MHz, CDCl₃-d) δ=8.38 (s, 1H), 7.01 (s, 1H), 2.74 (q, J=7.6 Hz, 2H), 2.33 (s, 3H), 1.26 (t, J=7.6 Hz, 3H).

### 1-(3-(2-(4-cyclopropylpiperazine-1-carbonyl)-7-fluoro-4-(4,4,5,5-tetramethyl-1,3,2-dioxaborolan-2-yl)-1H-indol-6-yl)-5,6-dihydropyridin-1(2H)-yl)-3-(1H-1,2,3-triazol-1-yl)propan-1-one (Intermediate OG)

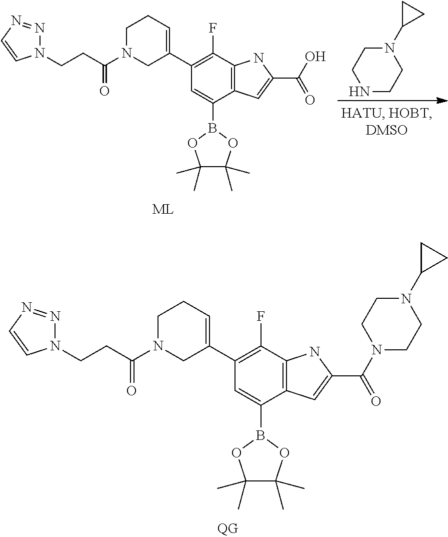

To a solution of 6-(1-(3-(1H-1,2,3-triazol-1-yl)propanoyl)-1,2,5,6-tetrahydropyridin-3-yl)-7-fluoro-4-(4,4,5,5-tetramethyl-1,3,2-dioxaborolan-2-yl)-1H-indole-2-carboxylic acid (900 mg, 1.77 mmol, Intermediate ML) and 1-cyclopropylpiperazine (223 mg, 1.77 mmol, CAS #20327-23-5) in DMSO (20 mL) was added HATU (1.01 g, 2.65 mmol), HOBt (358 mg, 2.65 mmol) and DIEA (685 mg, 5.3 mmol). The mixture was then stirred at 25° C. for 1 hr. Upon completion, the mixture was diluted with water (10 mL) and extracted with ethyl acetate (50 mL×3), dried over sodium sulfate, filtered and concentrated under reduced pressure. The residue was purified by column chromatography (SiO₂, DCM/THF=10/1 to 3/1) to give the title compound (1 g, 83% yield) as a white solid. LC-MS (ESI⁺) m/z 618.3 (M+H)⁺; ¹H NMR (400 MHz, CDCl₃-d) δ=9.51-9.27 (m, 1H), 7.77 (s, 1H), 7.68-7.63 (m, 1H), 7.53-7.45 (m, 1H), 7.20 (s, 1H), 6.21-6.00 (m, 1H), 4.80 (q, J=6.4 Hz, 2H), 4.44 (s, 1H), 4.32-4.16 (m, 1H), 3.98-3.86 (m, 3H), 3.82-3.66 (m, 3H), 3.56 (t, J=5.4 Hz, 1H), 3.10-2.99 (m, 2H), 2.77-2.71 (m, 4H), 2.62 (s, 8H), 2.37-2.31 (m, 2H), 1.91-1.63 (m, 3H), 1.38 (s, 12H), 0.56-0.45 (m, 4H).

### 3-Chloro-6-ethyl-2-methyl-pyridine (Intermediate QH)

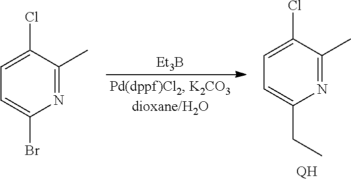

A mixture of 6-bromo-3-chloro-2-methyl-pyridine (4.8 g, 23 mmol, CAS #944317-27-5), Pd(dppf)Cl₂·CH₂Cl₂ (1.90 g, 2.3 mmol), K₂CO₃ (9.64 g, 69.7 mmol) and Et₃B (1 M, 23.2 mL) in dioxane (50 mL) and H₂O (10 mL) was degassed and purged with N₂ three times. Then the mixture was stirred at 80° C. for 2 hrs under N₂ atmosphere. Upon completion, the mixture was quenched with H₂O (10 mL) at 0° C., and then diluted with EA (50 mL) and extracted with EA (100 mL×3). The combined organic layers were washed with NaCl (50 mL×3), dried over Na₂SO₄, filtered and concentrated under reduced pressure to give a residue. The residue was purified by column chromatography (SiO₂, Petroleum ether/Ethyl acetate=1/0 to 50/1) to give the title compound (1.1 g, 30% yield) as a white solid. LC-MS (ESI+) m/z 156.1 (M+H)⁺.

### 3-Chloro-6-cyclopropyl-2-methyl-pyridine (Intermediate QI)

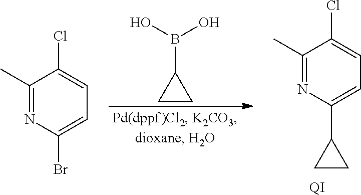

A mixture of 6-bromo-3-chloro-2-methyl-pyridine (1 g, 5 mmol, CAS #944317-27-5), cyclopropylboronic acid (2.08 g, 24.2 mmol, CAS #411235-57-9), Pd(dppf)Cl₂ (396 mg, 541 μmol) and K₂CO₃ (1.34 g, 9.69 mmol) in dioxane (10 mL) and H₂O (2 mL) was degassed and purged with N₂ three times. Then the mixture was stirred at 100° C. for 12 hrs under N₂ 32 atmosphere. Upon completion, the mixture was diluted with H₂O (30 mL) and extracted with EtOAc (20 mL×2). The combined organic layers were dried over Na₂SO₄, filtered and concentrated under reduced pressure to give a residue. The crude product was purified by reversed-phase HPLC (FA condition) to give the title compound (100 mg, 11% yield) as a yellow gum. LC-MS (ESI⁺) m/z 168.1/170.1 (M+1/M+2+1); ¹H NMR (400 MHz, DMSO-d₆) δ=7.65 (d, J=8.4 Hz, 1H), 7.13 (d, J=8.0 Hz, 1H), 2.44 (s, 3H), 2.12-2.00 (m, 1H), 1.00-0.81 (m, 4H).

### 5-Chloro-2-(difluoromethyl)-4-ethylpyridine (Intermediate QJ)

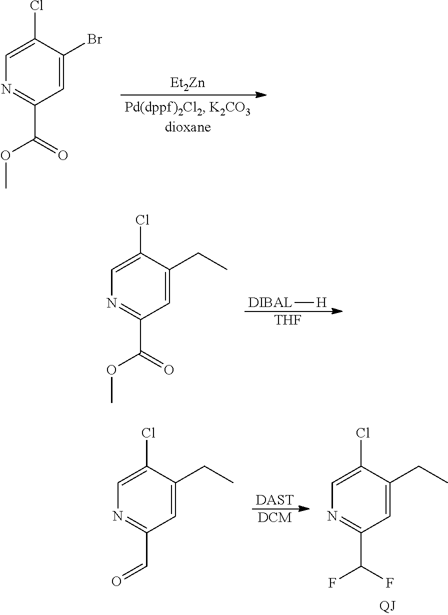

### Step 1—Methyl 5-chloro-4-ethylpicolinate

To a solution of methyl 4-bromo-5-chloro-pyridine-2-carboxylate (1 g, 4 mmol, CAS #1256834-36-2) in dioxane (15 mL) was added Pd(dppf)Cl₂ (146.06 mg, 199.62 μmol) K₂CO₃ (1.66 g, 12.0 mmol) and diethylzinc (1 M, 4.79 mL). The mixture was then stirred at 80° C. for 2 hr under N₂ atmosphere. Upon completion, the mixture was quenched with H₂O (20 mL) and extracted with EtOAc (20 mL×3). The organic layers was washed with brine (30 mL×2), dried over anhydrous sodium sulfate, filtered and concentrated under reduced pressure to give a residue. The crude was purified by column chromatography (SiO₂, PE/EtOAc=10/1 to 5/1) to give the title compound (160 mg, 18% yield) as a white solid. LC-MS (ESI⁺) m/z 200.1 (M+H)⁺; ¹H NMR (400 MHz, DMSO-d₆) δ=8.68 (s, 1H), 8.04 (s, 1H), 3.88 (s, 3H), 2.80 (q, J=7.6 Hz, 2H), 1.21 (t, J=7.6 Hz, 3H).

### Step 2—5-Chloro-4-ethylpicolinaldehyde

To a solution of methyl 5-chloro-4-ethyl-pyridine-2-carboxylate (160 mg, 802 μmol) in THF (10 mL) was added DIBAL-H (1 M, 1.20 mL). The mixture was then stirred at −78° C. for 1 hr under N₂ atmosphere. Upon completion, to the mixture was added water (0.1 mL) slowly followed by the addition of ethyl acetate (15 mL) and 15% aqueous sodium hydroxide (0.1 mL). Then water (0.3 mL) was added, and the mixture was warmed to rt and stirred for 15 min. Then Na₂SO₄ (50 mg) was added to the mixture, which was then was filtered and washed thoroughly with EtOAc (50 mL) to give the title compound (106 mg, 78% yield) as a yellow oil. LC-MS (ESI⁺) m/z 170.1 (M+H)⁺.

### Step 3—5-Chloro-2-(difluoromethyl)-4-ethylpyridine

To a solution of 5-chloro-4-ethyl-pyridine-2-carbaldehyde (106 mg, 625 μmol) in DCM (10 mL) was added DAST (201.48 mg, 1.25 mmol). The mixture was then stirred at −20° C. for 1 h. Upon completion, the mixture was diluted with sat. aq NH₄Cl (20 mL) and extracted with DCM (20 mL×3). The organic layers was washed with brine (40 mL×2), dried over anhydrous sodium sulfate, filtered and concentrated under reduced pressure to give a residue. The crude was purified by column chromatography (SiO₂, PE/EtOAc=100/1 to 10/1) to give the title compound (80 mg, 67% yield) as a colorless oil. LC-MS (ESI⁺) m/z 192.1 (M+H)⁺; ¹H NMR (400 MHz, DMSO-d₆) δ=9.00-8.65 (m, 1H), 7.89-7.69 (m, 1H), 7.18-6.79 (m, 1H), 2.88-2.74 (m, 2H), 1.33-1.17 (m, 3H).

### 5-Chloro-4-ethyl-2-methyl-pyrimidine (Intermediate QK)

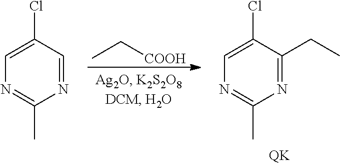

To a solution of 5-chloro-2-methyl-pyrimidine (800 mg, 6.22 mmol, CAS #54198-89-9) in DCM (10 mL) and H₂O (2 mL) was added K₂S₂O₈ (1.68 g, 6.22 mmol, 1.25 mL) and propionic acid (415 mg, 5.60 mmol). Then Ag₂O (288 mg, 1.24 mmol) was added under N₂ atmosphere and the mixture was stirred at 20° C. for 12 h. On completion, the reaction mixture was quenched by addition of sodium thiosulfate solution, and then diluted with water (20 mL) and extracted with DCM (150 mL). The combined organic layers were washed with brine (30 mL), dried over Na₂SO₄, filtered and concentrated under reduced pressure to give a residue. The residue was purified by prep-TLC (SiO₂, PE/EtOAc=10/1) to give the title compound (300 mg, 31% yield) as a colorless oil. LC-MS (ESI⁺) m/z 157.1 (M+H)⁺.

### 2-Bromothiazole (CAS #3034-53-5) (Intermediate OL)

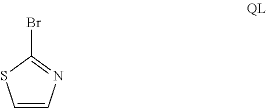

### 1-Ethyl-5-(4,45,5-tetramethyl-1,3,2-dioxaborolan-2-yl)pyrazole (CAS #1007110-53-3) (Intermediate QM)

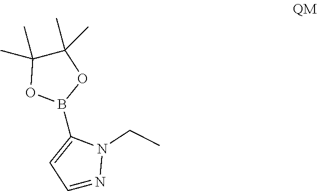 [SMILES: CCn1nccc1B1OC(C)(C)C(C)(C)O1]

### 1-(3-(4-Chloro-2-(1-ethyl-1,4,5,6-tetrahydropyrrolo[3,4-c]pyrazole-5-carbonyl)-7-fluoro-1H-indol-6-yl)-5,6-dihydropyridin-1(2H)-yl)-3-(1H-1,2,3-triazol-1-yl)propan-1-one (Intermediate QN)

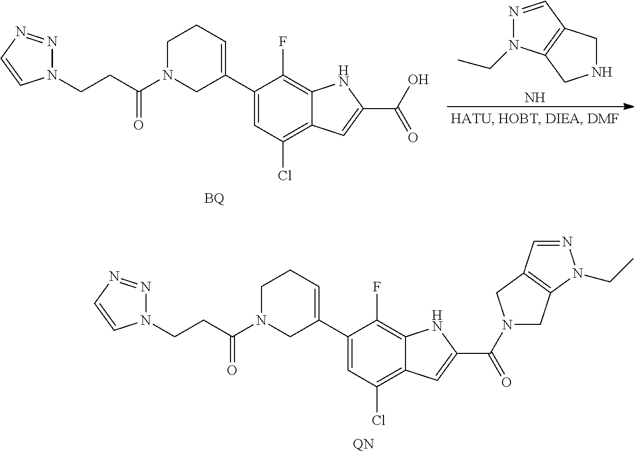

To a solution of 4-chloro-7-fluoro-6-[1-[3-(triazol-1-yl)propanoyl]-3,6-dihydro-2H-pyridin-5-yl]-1H-indole-2-carboxylic acid (200.00 mg, 478.67 μmol, Intermediate BQ), 1-ethyl-5,6-dihydro-4H-pyrrolo[3,4-c]pyrazole (65.67 mg, 478.67 μmol, Intermediate NH), HOBt (129.36 mg, 957.35 gmol), DIEA (371.19 mg, 2.87 mmol, 500.26 μL) in DMF (5 mL) was added HATU (273.01 mg, 718.01 gmol). The reaction mixture then was stirred at 20° C. for 0.25 h. Upon completion, the mixture was poured into H₂O (10 mL) and the mixture was filtered to get the filter cake. The crude product was triturated with H₂O (6 mL) at 20° C. for 10 min to give the title compound (260.00 mg, 96% yield) as an off-yellow solid. ¹H NMR (400 MHz, DMSO-d₆) δ=12.97-12.43 (m, 1H), 8.16-8.07 (m, 1H), 7.95 (s, 1H), 7.73-7.65 (m, 1H), 7.60-7.44 (m, 1H), 7.24-7.04 (m, 2H), 6.23-6.08 (m, 1H), 4.69-4.59 (m, 2H), 4.30 (d, J=18.0 Hz, 2H), 3.94-3.84 (m, 2H), 3.70-3.54 (m, 3H), 3.17-3.03 (m, 2H), 2.73 (s, 2H), 2.39-2.21 (m, 2H), 1.31-1.09 (m, 3H).

### 7-Bromo-2-(1-(tert-butoxycarbonyl)-1,2,5,6-tetrahydropyridin-3-yl)-4-fluoro-1H-indole-5-carboxylic acid (Intermediate QO)

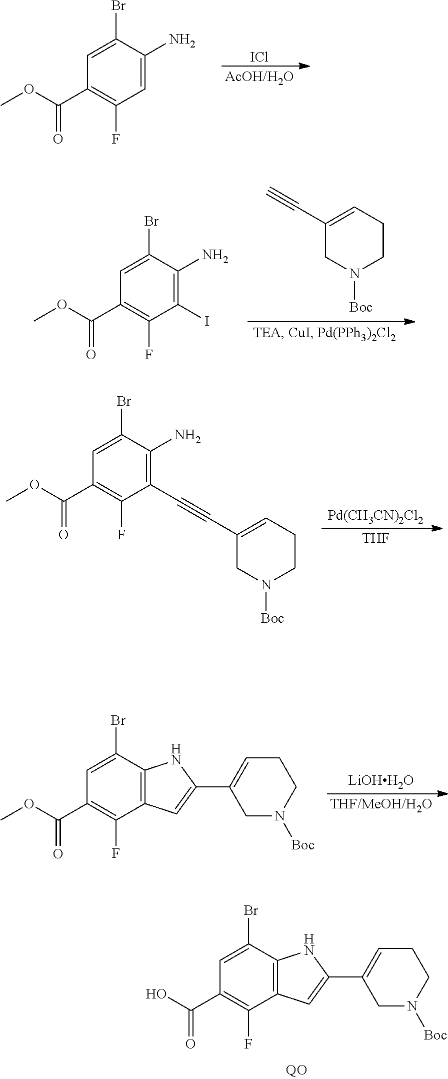

### Step 1—Methyl 4-amino-5-bromo-2-fluoro-3-iodobenzoate

To a solution of methyl 4-amino-5-bromo-2-fluoro-benzoate (2 g, 8 mmol, CAS #1427372-46-0) in HOAc (15 mL) and H₂O (15 mL) was added ClI (1.96 g, 12.1 mmol). The mixture was then stirred at 70° C. for 3 hrs. On completion, the reaction mixture was filtered and the filter cake was dried in vacuo to give the title compound (2.5 g) as a white solid. LC-MS (ESI⁺) m/z 373.8 (M+H)⁺; ¹H NMR (400 MHz, DMSO-d₆) δ=7.91 (d, J=7.6 Hz, 1H), 6.29 (s, 2H), 3.79 (s, 3H).

### Step 2—Tert-butyl3-((2-amino-3-bromo-6-fluoro-5-(methoxycarbonyl)phenyl)ethynyl)-5,6-dihydropyridine-1(2H)-carboxylate

A mixture of methyl 4-amino-5-bromo-2-fluoro-3-iodo-benzoate (2.4 g, 6.4 mmol), tert-butyl 5-ethynyl-3,6-dihydro-2H-pyridine-1-carboxylate (2.66 g, 12.8 mmol), Pd(PPh₃)₂Cl₂ (450 mg, 641 μmol), CuI (122 mg, 641 μmol) and TEA (3.25 g, 32.1 mmol) in DMF (25 mL) was degassed and purged with N₂ three times. Then the mixture was stirred at 50° C. for 2 hrs under N₂ atmosphere. On completion, the reaction mixture was concentrated in vacuo to give the crude residue. The residue was purified by column chromatography (SiO₂, Petroleum ether:Ethyl acetate=15/1 to 10/1) to give the title compound (2 g, 63% yield) as a brown solid. LC-MS (ESI⁺) m/z 475.0 (M+Na)⁺; ¹H NMR (400 MHz, DMSO-d₆) δ=7.85 (br d, J=7.2 Hz, 1H), 6.64-6.28 (m, 3H), 4.01 (br s, 2H), 3.78 (s, 3H), 3.44 (br s, 2H), 2.24 (br s, 2H), 1.47-1.39 (m, 9H).

### Step 3—Methyl 7-bromo-2-(1-(tert-butoxycarbonyl)-1,2,5,6-tetrahydropyridin-3-yl)-4-fluoro-1H-indole-5-carboxylate

A mixture of tert-butyl 5-[2-(2-amino-3-bromo-6-fluoro-5-methoxycarbonyl-phenyl)ethynyl]-3,6-dihydro-2H-pyridine-1-carboxylate (2 g, 4 mmol), Pd(CH₃CN)₂Cl₂ (114 mg, 441 μmol) in THF (25 mL) was degassed and purged with N₂ three times. Then the mixture was stirred at 80° C. for 1 hr under N₂ atmosphere. On completion, the reaction mixture was concentrated under reduced pressure to give a residue. The residue was purified by column chromatography (SiO₂, Petroleum ether:Ethyl acetate=6/1 to 4/1) to give the title compound (1.4 g, 65% yield) as a white solid. ¹H NMR (400 MHz, DMSO-d₆) δ=11.74 (br s, 1H), 7.73 (br d, J=6.0 Hz, 1H), 6.92-6.70 (m, 2H), 4.27 (br s, 2H), 3.86 (s, 3H), 3.50 (br s, 2H), 2.32 (br d, J=1.2 Hz, 2H), 1.45 (s, 9H).

### Step 4—7-Bromo-2-(1-(tert-butoxycarbonyl)-1,2,5,6-tetrahydropyridin-3-yl)-4-fluoro-1H-indole-5-carboxylic acid

To a solution of methyl 7-bromo-2-(1-tert-butoxycarbonyl-3,6-dihydro-2H-pyridin-5-yl)-4-fluoro-1H-indole-5-carboxylate (1.4 g, 3.1 mmol) in MeOH (4 mL), THF (12 mL), and H₂O (4 mL) was added LiOH H₂O (648 mg, 15.4 mmol). The mixture was then stirred at 50° C. for 12 hrs. On completion, the reaction mixture was added HCl (1N) until the pH was 5, then diluted with water (10 mL) and extracted with dichloromethane (3×30 mL). The combined organic layers were dried over anhydrous sodium sulfate, filtered and the filtrate was concentrated in vacuo to give the title compound (1.3 g) as an off-white solid. LC-MS (ESI⁺) m/z 439.0 (M+H)⁺.

### Tert-butyl 3-(5-(1-ethyl-1,4,5,6-tetrahydropyrrolo[3,4-c]pyrazole-5-carbonyl)-4-fluoro-7-(4,4,5,5-tetramethyl-1,3,2-dioxaborolan-2-yl)-1H-indol-2-yl)-5,6-dihydropyridine-1(2H)-carboxylate (Intermediate QP)

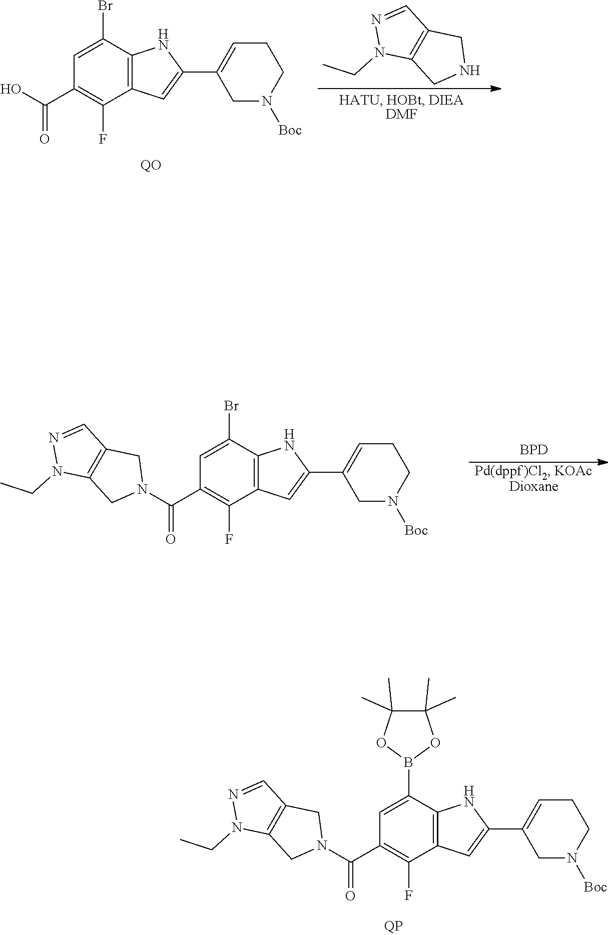

### Step 1—Tert-butyl3-(7-bromo-5-(1-ethyl-1,4,5,6-tetrahydropyrrolo[3,4-c]pyrazole-5-carbonyl)-4-fluoro-1H-indol-2-yl)-5,6-dihydropyridine-1(2H)-carboxylate

To a solution of 7-bromo-2-(1-tert-butoxycarbonyl-3,6-dihydro-2H-pyridin-5-yl)-4-fluoro-1H-indole-5-carboxylic acid (0.9 g, 2.1 mmol, Intermediate QO) in DMF (6 mL) was added HATU (856 mg, 2.25 mmol), HOBt (553 mg, 4.10 mmol) and DIEA (1.32 g, 10.2 mmol). Then 1-ethyl-5,6-dihydro-4H-pyrrolo[3,4-c]pyrazole (772 mg, 3.07 mmol, TFA, CAS #2281013-61-2) was added and the mixture was stirred at 20° C. for 2 hrs. On completion, the reaction mixture was quenched with water (10 mL) at 25° C., and then extracted with ethyl acetate (3×20 mL). The combined organic layers were dried over anhydrous sodium sulfate, filtered and the filtrate was concentrated in vacuo to give the crude residue. The residue was purified by column chromatography (SiO₂, Petroleum ether:Ethyl acetate=5/1 to 1/1) to give the title compound (0.9 g, 70% yield) as a white solid. LC-MS (ESI⁺) m/z 558.2 (M+H)⁺.

### Step 2—Tert-butyl3-(5-(1-ethyl-1,4,5,6-tetrahydropyrrolo[3,4-c]pyrazole-5-carbonyl)-4-fluoro-7-(4,4,5,5-tetramethyl-1,3,2-dioxaborolan-2-yl)-1H-indol-2-yl)-5,6-dihydropyridine-1(2H)-carboxylate

A mixture of tert-butyl 5-[7-bromo-5-(1-ethyl-4,6-dihydropyrrolo[3,4-c]pyrazole-5-carbonyl)-4-fluoro-1H-indol-2-yl]-3,6-dihydro-2H-pyridine-1-carboxylate (0.9 g, 1.6 mmol), 4,4,5,5-tetramethyl-2-(4,4,5,5-tetramethyl-1,3,2-dioxaborolan-2-yl)-1,3,2-dioxaborolane (1.02 g, 4.03 mmol, CAS #73183-34-3), Pd(dppf)Cl₂ CH₂Cl₂ (197 mg, 241 μmol), and KOAc (474 mg, 4.83 mmol) in dioxane (10 mL) was degassed and purged with N₂ three times. Then the mixture was stirred at 80° C. for 5 hrs under N₂ atmosphere. On completion, the reaction mixture was concentrated under reduced pressure to give a residue. The residue was purified by column chromatography (SiO₂, Petroleum ether:Ethyl acetate: DCM=2/2/1) to give the title compound (490 mg, 45% yield) as a brown solid. LC-MS (ESI⁺) m/z 606.4 (M+H)⁺; ¹H NMR (400 MHz, DMSO-d₆) δ=9.92 (br s, 1H), 7.49-7.10 (m, 2H), 6.80-6.41 (m, 2H), 4.78 (br s, 1H), 4.56 (br d, J=20.0 Hz, 2H), 4.31 (br d, J=7.6 Hz, 3H), 4.12 (br d, J=6.8 Hz, 1H), 4.01-3.91 (m, 1H), 3.53 (br s, 2H), 2.35 (br s, 2H), 1.46 (br s, 9H), 1.37 (br s, 12H), 1.08 (s, 3H).

### Tert-butyl 3-(5-(1-ethyl-1,4,5,6-tetrahydropyrrolo[3,4-c]pyrazole-5-carbonyl)-7-(2-ethylpyridin-3-yl)-4-fluoro-1H-indol-2-yl)-5,6-dihydropyridine-1(2H)-carboxylate (Intermediate QQ)

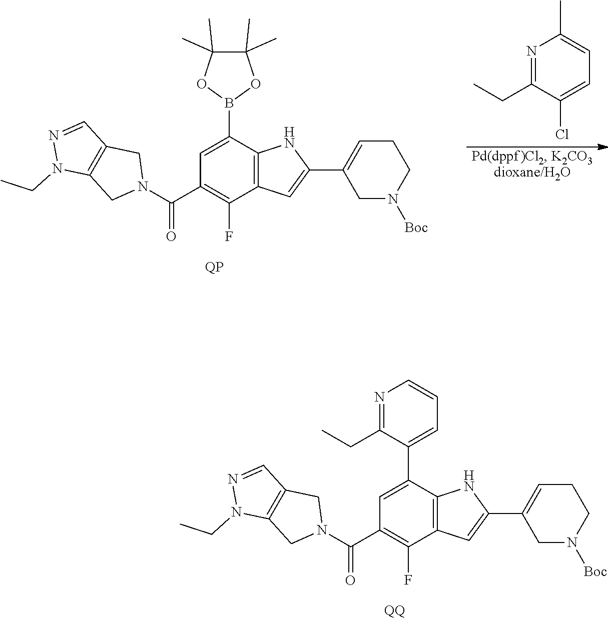

A mixture of tert-butyl 5-[5-(1-ethyl-4,6-dihydropyrrolo[3,4-c]pyrazole-5-carbonyl)-4-fluoro-7-(4,4,5,5-tetramethyl-1,3,2-dioxaborolan-2-yl)-1H-indol-2-yl]-3,6-dihydro-2H-pyridine-1-carboxylate (460 mg, 759 μmol, Intermediate QP), 3-chloro-2-ethyl-6-methyl-pyridine (141 mg, 911 μmol, CAS #1309256-58-3), XPhos Pd G3 (64.3 mg, 75.9 μmol), and K₂CO₃ (314 mg, 2.28 mmol) in dioxane (2 mL), H₂O (0.5 mL) was degassed and purged with N₂ three times. Then the mixture was stirred at 80° C. for 2 hrs under N₂ atmosphere. On completion, the reaction mixture was concentrated under reduced pressure to give a residue. The residue was purified by column chromatography (SiO₂, Petroleum ether: Ethyl acetate=3/1 to 1/1, DCM:EA=1/1) to give the title compound (350 g, 70% yield) as a brown solid. LC-MS (ESI⁺) m/z 599.3 (M+H)⁺.

### (1—(Cyclopropylmethyl)-7-(2-ethyl-6-methylpyridin-3-yl)-4-fluoro-2-(1,2,5,6-tetrahydropyridin-3-yl)-1H-indol-5-yl)(1-ethylpyrrolo[3,4-c]pyrazol-5(1H,4H,6H)-yl)methanone (Intermediate OR)

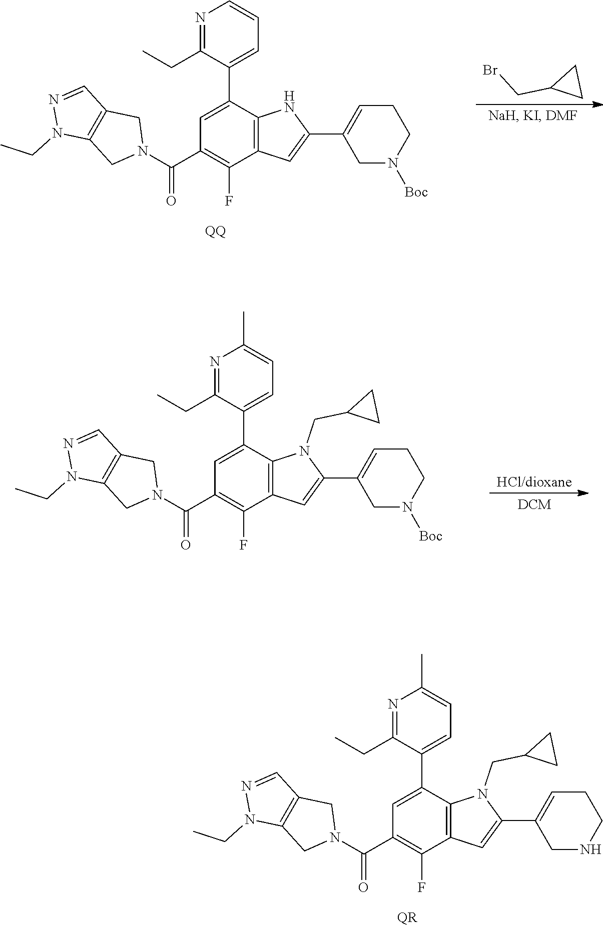

### Step 1—Tert-butyl3-(1-(cyclopropylmethyl)-5-(1-ethyl-1,4,5,6-tetrahydropyrrolo[3,4-c]pyrazole-5-carbonyl)-7-(2-ethyl-6-methylpyridin-3-yl)-4-fluoro-1H-indol-2-yl)-5,6-dihydropyridine-1(2H)-carboxylate

To a solution of tert-butyl 5-[5-(1-ethyl-4,6-dihydropyrrolo[3,4-c]pyrazole-5-carbonyl)-7-(2-ethyl-6-methyl-3-pyridyl)-4-fluoro-1H-indol-2-yl]-3,6-dihydro-2H-pyridine-1-carboxylate (300 mg, 501 μmol, Intermediate QQ) in DMF (5 mL) was added NaH (40.1 mg, 1.00 mmol, 60% dispersion in mineral oil) at 0° C., and the mixture was stirred at 0° C. for 0.5 hrs. Then bromomethylcyclopropane (101 mg, 751 μmol, CAS #7051-34-5), and KI (16.6 mg, 100 μmol) was added and the mixture was stirred at 25° C. for 1 hr. On completion, the reaction mixture was added NH₄Cl, then diluted with water (5 mL) and extracted with ethyl acetate (3×20 mL). The combined organic layers were dried over anhydrous sodium sulfate, filtered and the filtrate was concentrated in vacuo to give the crude residue. The residue was purified by column chromatography (SiO₂, Petroleum ether:Ethyl acetate=1/1, Dichloromethan:Ethyl acetate=1/1) to give the title compound (150 mg, 37% yield) as a gray solid. LC-MS (ESI⁺) m/z 653.4 (M+H)⁺

### Step 2—(1—(Cyclopropylmethyl)-7-(2-ethyl-6-methylpyridin-3-yl)-4-fluoro-2-(1,2,5,6-tetrahydropyridin-3-yl)-1H-indol-5-yl)(1-ethylpyrrolo[3,4-c]pyrazol-5(1H,4H,6H)-yl)methanone

To a solution of tert-butyl 5-[1-(cyclopropylmethyl)-5-(1-ethyl-4,6-dihydropyrrolo[3,4-c]pyrazole-5-carbonyl)-7-(2-ethyl-6-methyl-3-pyridyl)-4-fluoro-indol-2-yl]-3,6-dihydro-2H-pyridine-1-carboxylate (150 mg, 229 μmol) in DCM (4 mL) was added HCl/dioxane (4 M, 0.5 mL). The mixture was then stirred at 25° C. for 2 hrs. On completion, the reaction mixture was concentrated under reduced pressure to give the title compound (150 mg) as a white solid. LC-MS (ESI⁺) m/z 553.4 (M+H)⁺; ¹H NMR (400 MHz, DMSO-d₆) δ=9.69-9.45 (m, 2H), 8.48-8.31 (m, 1H), 7.70 (br d, J=7.6 Hz, 1H), 7.17-6.93 (m, 2H), 6.69 (s, 1H), 6.07-5.92 (m, 1H), 5.58 (s, 1H), 4.61 (br s, 1H), 4.47-4.37 (m, 3H), 3.98-3.90 (m, 3H), 3.84-3.73 (m, 5H), 2.71 (br s, 3H), 1.18 (brt, J=7.2 Hz, 2H), 1.06 (brt, J=7.2 Hz, 2H), 0.94-0.88 (m, 3H), 0.41-0.32 (m, 1H), 0.07-−0.05 (m, 3H), −0.14-−0.24 (m, 2H).

### 3-Methoxy-2-methyl-3-oxopropanoic acid (CAS #3097-74-3) (Intermediate OS)

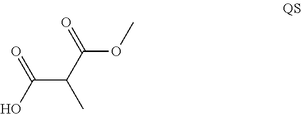

### (1S,2S)-cyclopropane-1,2-dicarboxylic acid (CAS #14590-54-6) (Intermediate OT)

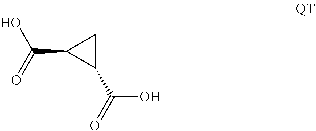

### Tert-butyl 3-(7-(6-ethyl-2-methylpyridin-3-yl)-5-(4-(5-fluoro-3-methoxypyridin-2-yl)piperazine-1-carbonyl)-1H-indol-2-yl)-5,6-dihydropyridine-1(2H)-carboxylate (Intermediate OU)

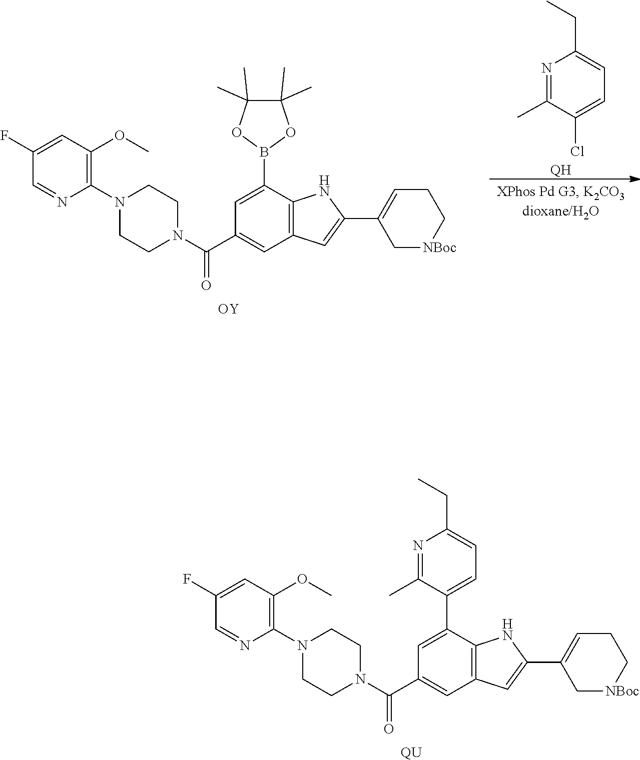

A mixture of tert-butyl 5-[5-[4-(5-fluoro-3-methoxy-2-pyridyl)piperazine-1-carbonyl]-7-(4,4,5,5-tetramethyl-1,3,2-dioxaborolan-2-yl)-1H-indol-2-yl]-3,6-dihydro-2H-pyridine-1-carboxylate (1.7 g, 2.6 mmol, Intermediate OY), 3-chloro-6-ethyl-2-methyl-pyridine (479 mg, 3.08 mmol, Intermediate QH), XPhos Pd G3 (217.51 mg, 256.96 μmol), K₂CO₃ (1.07 g, 7.71 mmol) in dioxane (20 mL) and H₂O (4 mL) was degassed and purged with N₂ three times. Then the mixture was stirred at 80° C. for 1 hr under N₂ atmosphere. The reaction mixture was filtered and concentrated under reduced pressure to give a residue. The residue was purified by column chromatography (SiO₂, Petroleum ether/Ethyl acetate=50/1 to 3/2) to give the title compound (1.31 g, 71% yield) as a yellow solid. LC-MS (ESI+) m/z 655.4 (M+H)⁺; ¹H NMR (400 MHz, DMSO-d₆) δ=10.84 (s, 1H), 7.78 (d, J=2.4 Hz, 1H), 7.64 (d, J=1.2 Hz, 1H), 7.57 (d, J=7.6 Hz, 1H), 7.35 (dd, J=2.4, 10.4 Hz, 1H), 7.19 (d, J=7.6 Hz, 1H), 6.93 (d, J=1.6 Hz, 1H), 6.65-6.55 (m, 2H), 4.24 (s, 2H), 3.83 (s, 3H), 3.66 (s, 4H), 3.47 (t, J=5.6 Hz, 2H), 3.24 (s, 4H), 2.81 (q, J=7.6 Hz, 2H), 2.25 (s, 3H), 2.25-2.20 (m, 2H), 1.44 (s, 9H), 1.31 (t, J=7.6 Hz, 3H).

### (1—(Cyclopropylmethyl)-7-(6-ethyl-2-methylpyridin-3-yl)-2-(1,2,5,6-tetrahydropyridin-3-yl)-1H-indol-5-yl)(4-(5-fluoro-3-methoxypyridin-2-yl)piperazin-1-yl)methanone (Intermediate QV)

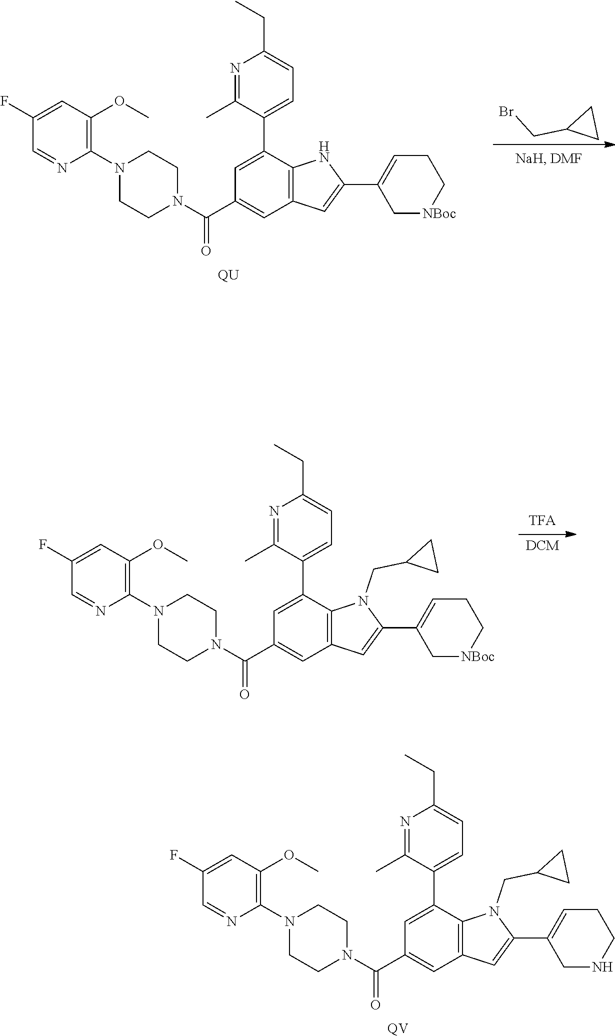

### Step 1—Tert-butyl 3-(1-(cyclopropylmethyl)-7-(6-ethyl-2-methylpyridin-3-yl)-5-(4-(5-fluoro-3-methoxypyridin-2-yl)piperazine-1-carbonyl)-1H-indol-2-yl)-5,6-dihydropyridine-1(2H)-carboxylate

To a solution of tert-butyl 5-[7-(6-ethyl-2-methyl-3-pyridyl)-5-[4-(5-fluoro-3-methoxy-2-pyridyl)piperazine-1-carbonyl]-1H-indol-2-yl]-3,6-dihydro-2H-pyridine-1-carboxylate (1 g, 2 mmol, Intermediate QU) in DMF (10 mL) was added NaH (91.6 mg, 2.29 mmol, 60% dispersion in mineral oil) at 0° C. and the mixture was stirred for 0.5 hr, followed addition of bromomethylcyclopropane (309 mg, 2.29 mmol, CAS #7051-34-5). The mixture was then stirred at 0-25° C. for 3.5 hrs. Upon completion, the mixture was quenched with NH₄Cl (20 mL) at 0° C., and then extracted with EA (8 mL×3). The combined organic layers were washed with aqueous NaCl (8 mL×2), dried over Na₂SO₄, filtered and concentrated under reduced pressure to give a residue. The residue was purified by column chromatography (SiO₂, Petroleum ether/Ethyl acetate=1/0 to 3/2) to give the title compound (1.1 g, 83% yield) as a yellow gum. LC-MS (ESI+) m/z 709.4 (M+H)⁺; ¹H NMR (400 MHz, DMSO-d₆) δ=7.78 (d, J=2.0 Hz, 1H), 7.74-7.66 (m, 2H), 7.38-7.32 (m, 1H), 7.25-7.18 (m, 1H), 6.94 (s, 1H), 6.64 (s, 1H), 6.06-5.96 (m, 1H), 4.24-4.11 (m, 1H), 4.05-3.97 (m, 1H), 3.84 (s, 3H), 3.67 (d, J=1.2 Hz, 4H), 3.26-3.24 (m, 4H), 2.54 (s, 3H), 2.54 (s, 2H), 2.26 (s, 1H), 2.20 (s, 3H), 1.41 (s, 9H), 1.28 (s, 2H), 1.25 (d, J=8.4 Hz, 2H), 0.87-0.80 (m, 1H), 0.46 (s, 1H), 0.14-0.05 (m, 2H), −0.22-−0.31 (m, 2H).

### Step 2—(1—(Cyclopropylmethyl)-7-(6-ethyl-2-methylpyridin-3-yl)-2-(1,2,5,6-tetrahydropyridin-3-yl)-1H-indol-5-yl)(4-(5-fluoro-3-methoxypyridin-2-yl)piperazin-1-yl)methanone

To a solution of tert-butyl 5-[1-(cyclopropylmethyl)-7-(6-ethyl-2-methyl-3-pyridyl)-5-[4-(5-fluoro-3-methoxy-2-pyridyl)piperazine-1-carbonyl]indol-2-yl]-3,6-dihydro-2H-pyridine-1-carboxylate (1.1 g, 1.6 mmol) in DCM (10 mL) was added TFA (4.61 g, 40.3 mmol). The mixture was then stirred at 25° C. for 0.5 hr. Upon completion, the mixture was concentrated under reduced pressure to give the title compound (1.1 g, TFA) as a brown gum. LC-MS (ESI+) m/z 609.4 (M+H)⁺.

### (1R,2S)-2-methoxycarbonylcyclopropanecarboxylic acid (CAS #31420-47-0) (Intermediate OW)

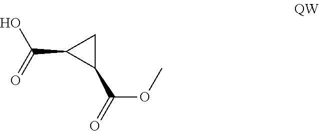

### Ethyl 3-chloro-3-oxo-propanoate (CAS #36239-09-5) (Intermediate OX)

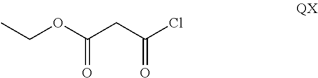

### Methyl 2-chloro-2-oxoacetate (CAS #5781-53-3) (Intermediate QY)

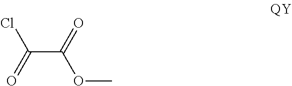

### Tert-butyl 3-(7-(2-ethylpyridin-3-yl)-5-(4-(5-fluoro-3-methoxypyridin-2-yl)piperazine-1-carbonyl)-1H-indol-2-yl)-5,6-dihydropyridine-1(2H)-carboxylate (Intermediate QZ)

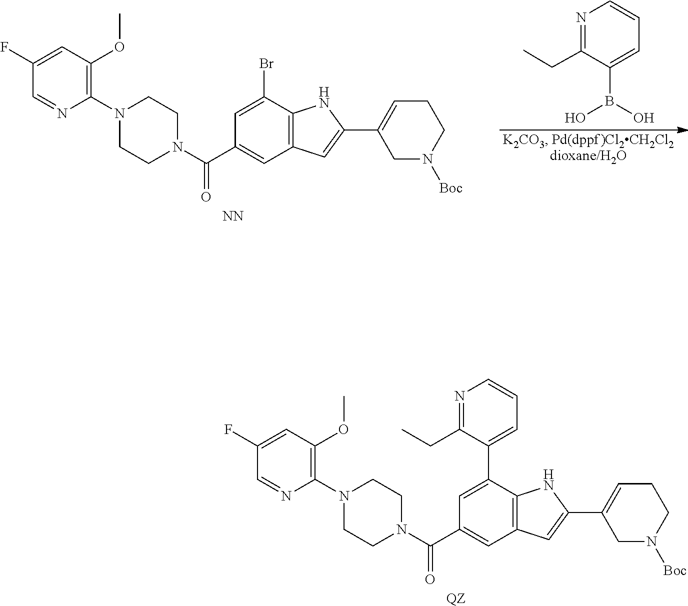

A mixture of tert-butyl 3-(7-bromo-5-(4-(5-fluoro-3-methoxypyridin-2-yl)piperazine-1-carbonyl)-1H-indol-2-yl)-5,6-dihydropyridine-1(2H)-carboxylate (600 mg, 976 μmol, Intermediate NN), (2-ethylpyridin-3-yl)boronic acid (147 mg, 976 μmol, CAS #1310384-02-1), K₂CO₃ (405 mg, 2.93 mmol) and Pd(dppf)Cl₂·CH₂Cl₂ (79.7 mg, 97.6 μmol) in dioxane (4 mL) and H₂O (0.5 mL) was degassed and purged with N₂ three times. Then the mixture was stirred at 80° C. for 12 hrs under N₂ atmosphere. On completion, the reaction mixture was concentrated in vacuo to give the crude residue. The residue was purified by column chromatography (SiO₂, Petroleum ether:Ethyl acetate=1/0 to 0/1) to give the title compound (400 mg, 58% yield) as a yellow solid. LC-MS (ESI+) m/z 641.2 (M+H)⁺; ¹H NMR (400 MHz, DMSO-d6) δ=10.85 (s, 1H), 8.61 (dd, J=1.6, 4.8 Hz, 1H), 7.78 (d, J=2.4 Hz, 1H), 7.67-7.61 (m, 2H), 7.37-7.31 (m, 2H), 6.93 (d, J=1.2 Hz, 1H), 6.62-6.55 (m, 2H), 4.23 (br s, 2H), 3.83 (s, 3H), 3.66 (br s, 4H), 3.46 (br t, J=5.2 Hz, 2H), 3.24 (br s, 4H), 2.64-2.54 (m, 2H), 2.23 (br s, 2H), 1.43 (s, 9H), 1.06 (t, J=7.6 Hz, 3H).

### (1—(Cyclopropylmethyl)-7-(2-ethylpyridin-3-yl)-2-(1,2,5,6-tetrahydropyridin-3-yl)-1H-indol-5-yl)(4-(5-fluoro-3-methoxypyridin-2-yl)piperazin-1-yl)methanone (Intermediate RA)

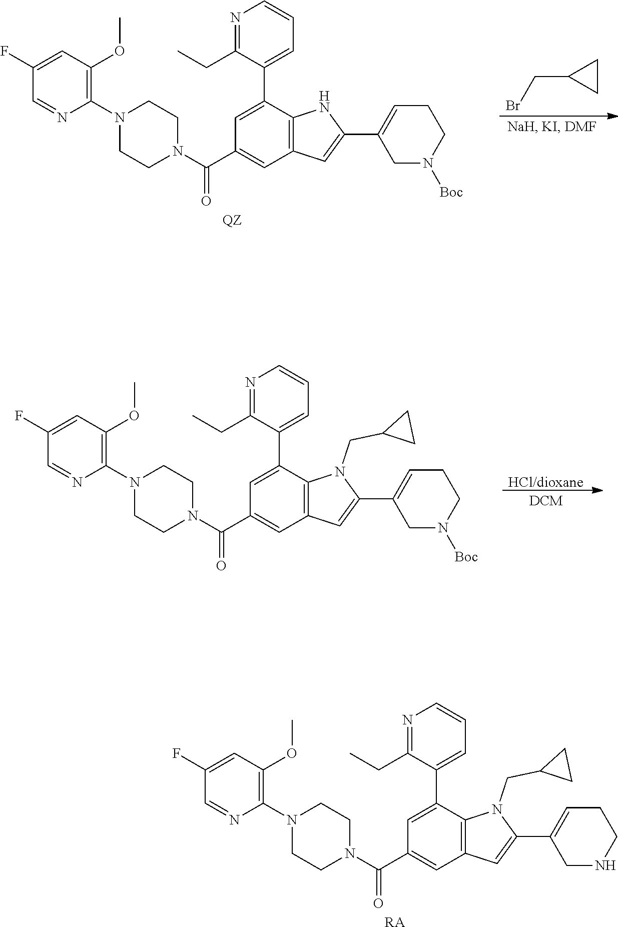

### Step 1—Tert-butyl 3-(1-(cyclopropylmethyl)-7-(2-ethylpyridin-3-yl)-5-(4-(5-fluoro-3-methoxypyridin-2-yl)piperazine-1-carbonyl)-1H-indol-2-yl)-5,6-dihydropyridine-1(2H)-carboxylate

To a solution of tert-butyl 3-(7-(2-ethylpyridin-3-yl)-5-(4-(5-fluoro-3-methoxypyridin-2-yl)piperazine-1-carbonyl)-1H-indol-2-yl)-5,6-dihydropyridine-1(2H)-carboxylate (400 mg, 624 μmol, Intermediate QZ) in DMF (5 mL) was added NaH (49.9 mg, 1.25 mmol, 60% dispersion in mineral) at 0° C. for 1 hr. Then added KI (10.4 mg, 62.4 μmol) and (bromomethyl)cyclopropane (126 mg, 936 μmol, 89.40 μL) to the mixture and stirred at 25° C. for 11 hrs. On completion, the reaction mixture was quenched with ice water (10 mL) at 0° C. and extracted with ethyl acetate (3×20 mL). The combined organic layers were washed with brine (10 mL) and dried over anhydrous sodium sulfate, filtered and the filtrate was concentrated in vacuo to give the crude residue. The residue was purified by column chromatography (SiO₂, Petroleum ether:Ethyl acetate=10/1 to 1/0) to give the title compound (220 mg, 41% yield) as a yellow oil. LC-MS (ESI+) m/z 695.4 (M+H)⁺; ¹H NMR (400 MHz, DMSO-d6) δ=8.63 (br d, J=3.2 Hz, 1H), 7.84-7.75 (m, 2H), 7.75-7.69 (m, 1H), 7.39-7.33 (m, 2H), 6.95 (s, 1H), 6.65 (s, 1H), 5.98 (br s, 1H), 4.19 (br d, J=18.4 Hz, 1H), 3.84 (s, 3H), 3.63-3.63 (m, 1H), 3.67 (br d, J=1.2 Hz, 3H), 3.56-3.42 (m, 3H), 3.25 (br s, 4H), 2.26 (br s, 2H), 1.41 (br s, 9H), 1.09-1.04 (m, 3H), 0.47 (br s, 1H), 0.15-0.06 (m, 2H), −0.23 (br s, 2H).

### Step 2—(1—(Cyclopropylmethyl)-7-(2-ethylpyridin-3-yl)-2-(1,2,5,6-tetrahydropyridin-3-yl)-1H-indol-5-yl)(4-(5-fluoro-3-methoxypyridin-2-yl)piperazin-1-yl)methanone

To a solution of tert-butyl 3-(1-(cyclopropylmethyl)-7-(2-ethylpyridin-3-yl)-5-(4-(5-fluoro-3-methoxypyridin-2-yl)piperazine-1-carbonyl)-1H-indol-2-yl)-5,6-dihydropyridine-1(2H)-carboxylate (220 mg, 317 μmol) in DCM (20 mL) was added HCl/dioxane (8 M, 5.50 mL). The mixture was then stirred at 25° C. for 0.5 hr. On completion, the reaction mixture was concentrated in vacuo to give the title compound (220 mg, HCl) as a yellow solid. LC-MS (ESI+) m/z 595.3 (M+H)⁺.

### 5-Bromo-4-ethyl-1,2,3,6-tetrahydropyridine (Intermediate R^{B})

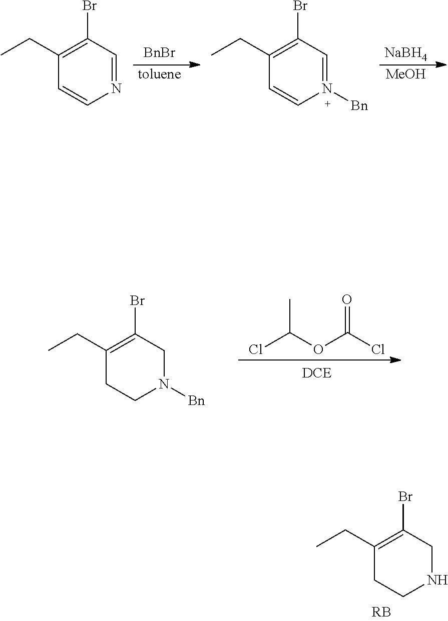

### Step 1—1-Benzyl-3-bromo-4-ethylpyridin-1-ium

To a solution of 3-bromo-4-ethyl-pyridine (1 g, 5 mmol, CAS #38749-76-7) in toluene (10 mL) was added bromomethylbenzene (1.01 g, 5.91 mmol, CAS #100-39-0). The mixture was then stirred at 110° C. for 20 hrs. Upon completion, the mixture was filtered and concentrated under reduced pressure to give the title compound (1.5 g, 81% yield) as an off red solid. ¹H NMR (400 MHz, CDCl₃-d) δ=9.75 (dd, J=1.2, 6.4 Hz, 1H), 9.60 (d, J=1.2 Hz, 1H), 7.86 (d, J=6.4 Hz, 1H), 7.76-7.69 (m, 2H), 7.40-7.37 (m, 3H), 6.38 (s, 2H), 2.89 (q, J=7.6 Hz, 2H), 1.29 (t, J=7.6 Hz, 3H).

### Step 2—1-Benzyl-5-bromo-4-ethyl-1,2,3,6-tetrahydropyridine

To a solution of 1-benzyl-3-bromo-4-ethyl-pyridin-1-ium (1.4 g, 5.1 mmol) in MeOH (15 mL) was added NaBH₄ (955 mg, 25.2 mmol) at 0° C. slowly. The mixture was then stirred at 0-25° C. for 2 hrs. Upon completion, the mixture was quenched with H₂O (50 mL) at 0° C., and then extracted with EA (50 mL×3). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=1/0 to 10/1) to give the title compound (700 mg, 45% yield) as a yellow oil. LC-MS (ESI⁺) m/z 280.0 (M+H)⁺; ¹H NMR (400 MHz, CDCl₃-d) δ=7.42-7.27 (m, 5H), 3.63 (s, 2H), 3.26 (s, 2H), 2.66 (s, 2H), 2.31-2.16 (m, 4H), 1.01 (t, J=7.6 Hz, 3H).

### Step 3—5-Bromo-4-ethyl-1,2,3,6-tetrahydropyridine

To a solution of 1-benzyl-5-bromo-4-ethyl-3,6-dihydro-2H-pyridine (400 mg, 1.43 mmol) in DCE (6 mL) was added 1-chloroethyl carbonochloridate (224 mg, 1.57 mmol, CAS #50893-53-3) at 0° C. slowly. The mixture was then stirred at 0-84° C. for 2 hrs. Upon completion, the mixture was filtered and concentrated under reduced pressure to give a residue. The residue was purified by column chromatography (SiO₂, DCM:MeOH=10:1) to give the title compound (170 mg, 63% yield) as a white solid. ¹H NMR (400 MHz, CDCl₃-d) δ=3.78 (s, 2H), 3.25 (t, J=5.8 Hz, 2H), 2.53-2.43 (m, 2H), 2.25-2.17 (m, 2H), 0.97 (t, J=7.6 Hz, 3H).

### Methyl 6-chloro-4-(difluoromethyl)-7-fluoro-1H-indole-2-carboxylate (Intermediate RC)

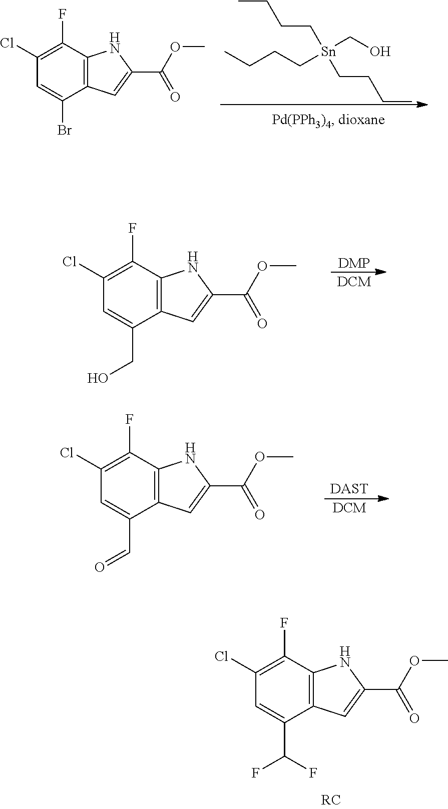

### Step 1—Methyl 6-chloro-7-fluoro-4-(hydroxymethyl)-1H-indole-2-carboxylate

A mixture of methyl 4-bromo-6-chloro-7-fluoro-1H-indole-2-carboxylate (1 g, 3 mmol, synthesized via Steps 1-5 of Intermediate C), (tributylstannyl)methanol (1.68 g, 5.22 mmol, CAS #27490-33-1), palladium triphenylphosphane (120 mg, 326 μmol) in dioxane (20 mL) was degassed and purged with N₂ three times. Then the mixture was stirred at 100° C. for 12 hrs under N₂ atmosphere. On completion, the reaction mixture was filtered and concentrated under reduced pressure to give a residue. The crude residue was purified by prep-HPLC (neutral condition) to give the title compound (0.5 g, 43% yield) as a white solid. LC-MS (ESI⁺) m/z 256.0 (M−H)⁺.

### Step 2—Methyl 6-chloro-7-fluoro-4-formyl-1H-indole-2-carboxylate

A mixture of methyl 6-chloro-7-fluoro-4-(hydroxymethyl)-1H-indole-2-carboxylate (0.3 g, 1.2 mmol) in DCM (3 mL) at 0° C. was added DMP (987 mg, 2.33 mmol, 721 μL) degassed and purged with N₂ three times. Then the mixture was stirred at 25° C. for 3 hrs under N₂ atmosphere. On completion, the mixture was quenched by addition of Na₂SO₃ (5 mL) at 0° C., and then extracted with DCM (10 mL×3). The combined organic layers were dried over Na₂SO₄, filtered and concentrated under reduced pressure to give a residue. Then DMF (5 mL) was added to the reaction mixture, then the crude product was triturated with H₂O at 0° C. for 15 min to give the title compound (0.2 g) as a white solid. ¹H NMR (400 MHz, DMSO-d₆) δ=13.25 (br s, 1H), 10.14 (s, 1H), 7.99 (d, J=6.4 Hz, 1H), 7.76 (d, J=2.8 Hz, 1H), 3.92 (s, 3H).

### Step 3—Methyl 6-chloro-4-(difluoromethyl)-7-fluoro-1H-indole-2-carboxylate

A mixture of methyl 6-chloro-7-fluoro-4-formyl-1H-indole-2-carboxylate (0.2 g, 780 μmol) in DCM (1 mL) at 0° C. was added DAST (756 mg, 4.69 mmol, 620 μL), and then the mixture was stirred at 0° C. for 96 hours under N₂ atmosphere. On completion, the reaction mixture was quenched by addition of NaHCO₃ (10 mL) at 0° C., and then extracted with DCM (10 mL×3). The combined organic layers were dried over Na₂SO₄, filtered and concentrated under reduced pressure to give a residue and purified by prep-HPLC (neutral condition) to give the title compound (0.11 g, 50% yield) as a white solid. LC-MS (ESI⁺) m/z 275.8 (M+H)⁺.

### 6-(1-(3-(1H-1,2,3-triazol-1-yl)propanoyl)-1,2,5,6-tetrahydropyridin-3-yl)-4-(difluoromethyl)-7-fluoro-1H-indole-2-carboxylic acid (Intermediate RD)

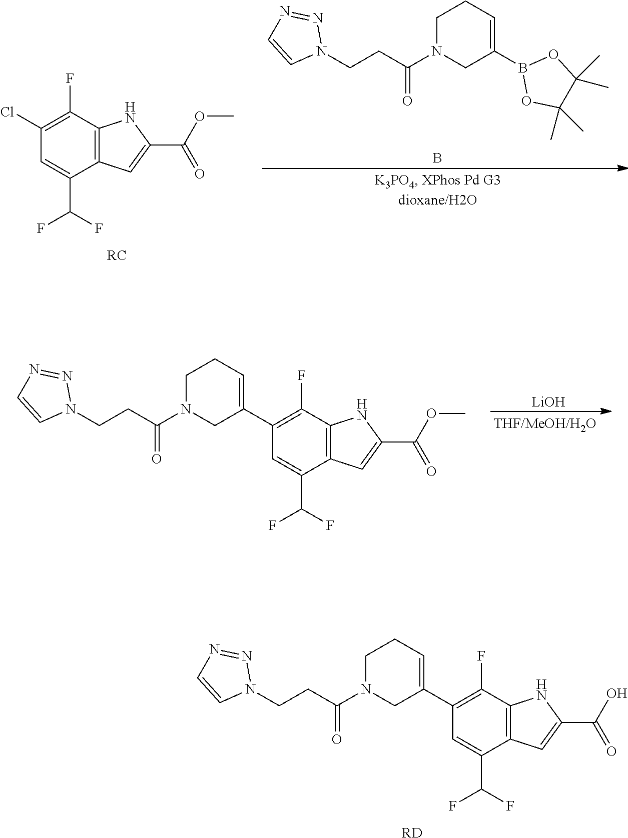

### Step 1—Methyl 6-(1-(3-(1H-1,2,3-triazol-1-yl)propanoyl)-1,2,5,6-tetrahydropyridin-3-yl)-4-(difluoromethyl)-7-fluoro-1H-indole-2-carboxylate

A mixture of 1-(3-(4,4,5,5-tetramethyl-1,3,2-dioxaborolan-2-yl)-5,6-dihydropyridin-1(2H)-yl)-3-(1H-1,2,3-triazol-1-yl)propan-1-one (143 mg, 432 μmol, Intermediate B), methyl 6-chloro-4-(difluoromethyl)-7-fluoro-1H-indole-2-carboxylate (100 mg, 360 μmol, Intermediate RC), XPhos Pd G3 (30.4 mg, 36 μmol), K₃PO₄ (229 mg, 1.08 mmol) in dioxane (2 mL) and H₂O (0.5 mL) was degassed and purged with N₂ three times. Then the mixture was stirred at 80° C. for 2 hrs under N₂ atmosphere. On completion, the reaction mixture was triturated with H₂O at 0° C. for 15 min to give the title compound (0.15 g) as a yellow solid. LC-MS (ESI⁺) m/z 895.5 (2M+H)⁺.

### Step 2—6-(1-(3-(1H-1,2,3-triazol-1-yl)propanoyl)-1,2,5,6-tetrahydropyridin-3-yl)-4-(difluoromethyl)-7-fluoro-1H-indole-2-carboxylic acid

To a solution of methyl 6-(1-(3-(1H-1,2,3-triazol-1-yl)propanoyl)-1,2,5,6-tetrahydropyridin-3-yl)-4-(difluoromethyl)-7-fluoro-1H-indole-2-carboxylate (100 mg, 223 μmol) in THE (2 mL), MeOH (0.5 mL) and H₂O (0.5 mL) was added LiOH (23.4 mg, 558 μmol). Then the mixture was stirred at 25° C. for 12 hrs. On completion, the reaction mixture was filtered and concentrated under reduced pressure to give a residue and purified by prep-HPLC (neutral condition) to give the title compound (30 mg, 24% yield) as a white solid. LC-MS (ESI⁺) m/z 442.0 (M+H)⁺.

### Tert-butyl 3-(7-(3-ethylpyridin-4-yl)-5-(4-(5-fluoro-3-methoxypyridin-2-yl)piperazine-1-carbonyl)-1H-indol-2-yl)-5,6-dihydropyridine-1(2H)-carboxylate (Intermediate RE)

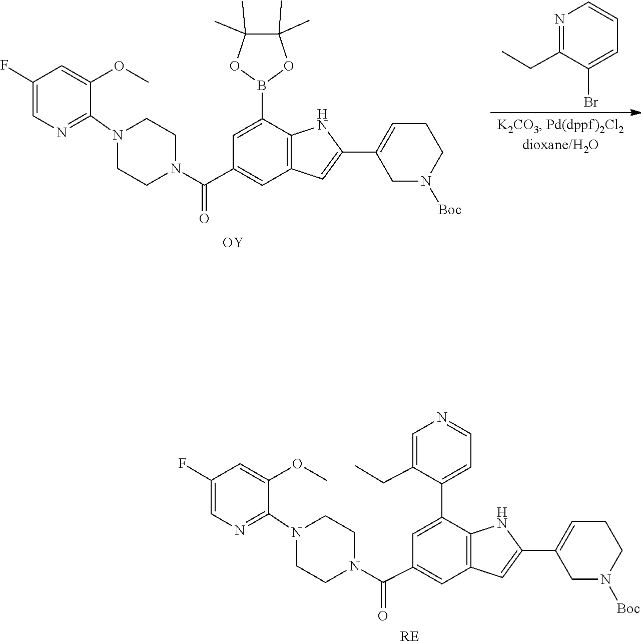

A mixture of 4-bromo-3-ethylpyridine (90.8 mg, 408 μmol, HCl, CAS #10168-60-2), tert-butyl 3-(5-(4-(5-fluoro-3-methoxypyridin-2-yl)piperazine-1-carbonyl)-7-(4,4,5,5-tetramethyl-1,3,2-dioxaborolan-2-yl)-1H-indol-2-yl)-5,6-dihydropyridine-1(2H)-carboxylate (180 mg, 272 μmol, Intermediate OY), Pd(dppf)Cl₂·CH₂Cl₂ (22.2 mg, 27.2 μmol), and K₂CO₃ (112 mg, 816 μmol) in dioxane (0.5 mL) and H₂O (0.1 mL) was degassed and purged with N₂ three times. Then the mixture was stirred at 80° C. for 2 hrs under N₂ atmosphere. On completion, the reaction mixture was quenched with 10% sodium sulfite solution (20 mL) and extracted with dichloromethane (3×20 mL). The combined organic layers were washed with brine (25 mL) and dried over anhydrous sodium sulfate, filtered and the filtrate was concentrated in vacuo to give the residue. The residue was purified by column chromatography (SiO₂, Petroleum ether:Ethyl acetate=100/1 to 0/1) to give the title compound (120 mg) as a yellow solid. LC-MS (ESI⁺) m/z 641.4 (M+H)⁺.

### (1—(Cyclopropylmethyl)-7-(3-ethylpyridin-4-yl)-2-(1,2,5,6-tetrahydropyridin-3-yl)-1H-indol-5-yl)(4-(5-fluoro-3-methoxypyridin-2-yl)piperazin-1-yl)methanone (Intermediate RF)

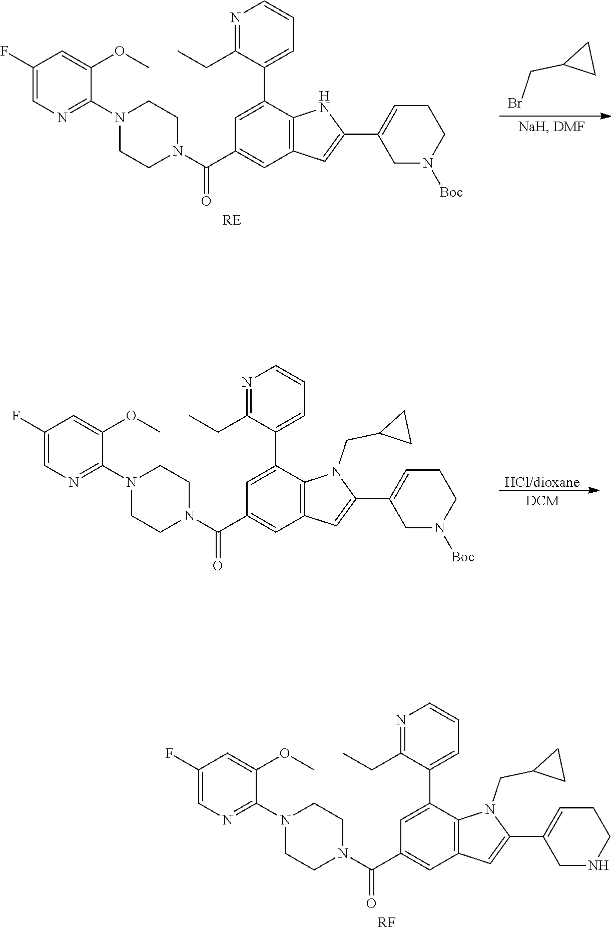

### Step 1—Tert-butyl 3-(1-(cyclopropylmethyl)-7-(3-ethylpyridin-4-yl)-5-(4-(5-fluoro-3-methoxypyridin-2-yl)piperazine-1-carbonyl)-1H-indol-2-yl)-5,6-dihydropyridine-1(2H)-carboxylate

To a solution of tert-butyl 3-(7-(3-ethylpyridin-4-yl)-5-(4-(5-fluoro-3-methoxypyridin-2-yl)piperazine-1-carbonyl)-1H-indol-2-yl)-5,6-dihydropyridine-1(2H)-carboxylate (80.0 mg, 124 μmol, Intermediate RE) in DMF (2 mL) was added NaH (7.49 mg, 187 μmol, 60% dispersion in mineral oil) and (bromomethyl)cyclopropane (20.2 mg, 149 μmol) under N₂. The mixture was then stirred at 0° C. for 2 hrs. On completion, the reaction mixture was quenched with water (20 mL) and extracted with ethyl acetate (3×20 mL). The combined organic layers were washed with brine (20 mL) and dried over anhydrous sodium sulfate, filtered and the filtrate was concentrated in vacuo to give the crude residue. The crude residue was purified by reverse-phase HPLC (0.8 g/L ammonium bicarbonate) to give the title compound (80.0 mg) as a yellow solid. LC-MS (ESI⁺) m/z 695.4 (M+H)⁺.

### Step 2—(1-Cyclopropylmethyl)-7-(3-ethylpyridin-4-yl)-2-(1,2,5,6-tetrahydropyridin-3-yl)-1H-indol-5-yl)(4-(5-fluoro-3-methoxypyridin-2-yl)piperazin-1-yl)methanone

A mixture of tert-butyl 3-(1-(cyclopropylmethyl)-7-(3-ethylpyridin-4-yl)-5-(4-(5-fluoro-3-methoxypyridin-2-yl)piperazine-1-carbonyl)-1H-indol-2-yl)-5,6-dihydropyridine-1(2H)-carboxylate (80.0 mg, 115 μmol) in HCl/dioxane (2 mL) and DCM (2 mL) was degassed and purged with N₂ three times. Then the mixture was stirred at 25° C. for 2 hrs. On completion, the reaction mixture was concentrated in vacuo to give the title compound (60.0 mg) as a yellow solid.

### Tert-butyl 3-(7-bromo-5-(4-(5-fluoro-3-methoxy-6-methylpyridin-2-yl) piperazine-1-carbonyl)-1H-indol-2-yl)-5,6-dihydropyridine-1(2H)-carboxylate (Intermediate RG)

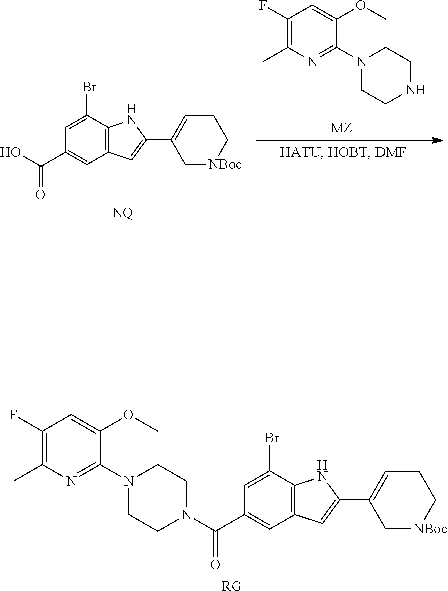

To a solution of 7-bromo-2-(1-tert-butoxycarbonyl-3,6-dihydro-2H-pyridin-5-yl)-1-(cyclopropylmethyl)indole-5-carboxylic acid (1.2 g, 2.5 mmol, Intermediate NQ) in DMF (16 mL) was added HATU (1.15 g, 3.03 mmol), HOBt (682 mg, 5.05 mmol), DIEA (1.30 g, 10.10 mmol, 1.76 mL), and 1-(5-fluoro-3-methoxy-6-methyl-2-pyridyl)piperazine (792 mg, 3.03 mmol, Intermediate MZ). The mixture was then stirred at 25° C. for 0.2 hr. On completion, the reaction mixture was quenched with H₂O (10 mL), and then filtered and concentrated under reduced pressure to give a residue. The crude product was purified by re-crystallization from PE (50 mL) at 25° C. to give the title compound (1 g, 57% yield) as a yellow solid. LC-MS (ESI+) m/z 628.2 (M+H)⁺; ¹H NMR (400 MHz, DMSO-d₆) δ=11.25 (s, 1H), 7.63 (s, 1H), 7.36 (d, J=1.2 Hz, 1H), 7.30 (d, J=10.8 Hz, 1H), 6.63 (s, 1H), 4.26 (d, J=1.2 Hz, 2H), 3.81 (s, 3H), 3.64 (s, 3H), 3.51 (t, J=5.2 Hz, 2H), 3.24 (s, 4H), 2.33 (d, J=5.2 Hz, 2H), 2.27 (d, J=2.8 Hz, 3H), 1.45 (s, 11H)

### Tert-butyl 3-(5-(4-(5-fluoro-3-methoxy-6-methylpyridin-2-yl) piperazine-1-carbonyl)-7-(4,4,55-tetramethyl-1,3,2-dioxaborolan-2-yl)-1H-indol-2-yl)-5,6-dihydropyridine-1(2H)-carboxylate (Intermediate RH)

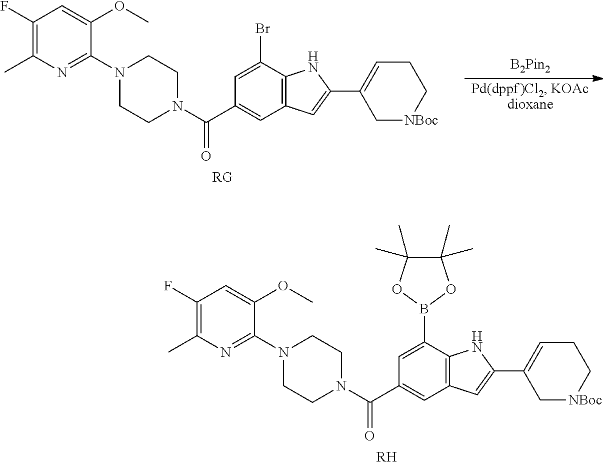

A mixture of tert-butyl 5-[7-bromo-5-[4-(5-fluoro-3-methoxy-6-methyl-2-pyridyl)piperazine-1-carbonyl]-1H-indol-2-yl]-3,6-dihydro-2H-pyridine-1-carboxylate (1 g, 2 mmol, Intermediate RG), KOAc (468 mg, 4.77 mmol), 4,4,5,5-tetramethyl-2-(4,4,5,5-tetramethyl-1,3,2-dioxaborolan-2-yl)-1,3,2-dioxaborolane (2.02 g, 7.96 mmol) and Pd(dppf)Cl₂ (116 mg, 159 μmol) in dioxane (13 mL) was degassed and purged with N₂ three times. Then the mixture was stirred at 80° C. for 4 hrs under N₂ atmosphere. On completion, the reaction mixture was filtered and concentrated under reduced pressure to give a residue. The residue was purified by column chromatography (SiO₂, Petroleum ether/Ethyl acetate=10/1 to 2/1) to give the title compound (600 mg, 25% yield) as a yellow oil. LC-MS (ESI⁺) m/z 676.4 (M+H)⁺.

### Tert-butyl 3-(7-(2-ethyl-6-methylpyridin-3-yl)-5-(4-(5-fluoro-3-methoxy-6-methylpyridin-2-yl) piperazine-1-carbonyl)-1H-indol-2-yl)-5,6-dihydropyridine-1(2H)-carboxylate (Intermediate RI)

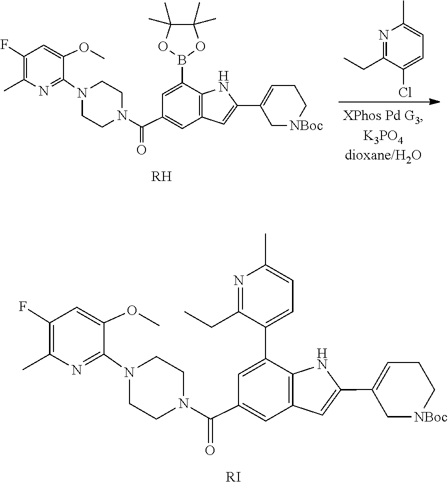

A mixture of tert-butyl 5-[5-[4-(5-fluoro-3-methoxy-6-methyl-2-pyridyl)piperazine-1-carbonyl]-7-(4,4,5,5-tetramethyl-1,3,2-dioxaborolan-2-yl)-1H-indol-2-yl]-3,6-dihydro-2H-pyridine-1-carboxylate (600 mg, 888 μmol, Intermediate RH), 3-chloro-2-ethyl-6-methyl-pyridine (165 mg, 1.07 mmol, CAS #1309256-58-3), XPhos Pd G3 (75.1 mg, 88.8 μmol), and K₂CO₃ (368 mg, 2.66 mmol) in dioxane (1 mL), H₂O (0.2 mL) was degassed and purged with N₂ three times. Then the mixture was stirred at 80° C. for 1 hr under N₂ atmosphere. On completion, filtered and concentrated under reduced pressure to give a residue. The residue was purified by column chromatography (SiO₂, Petroleum ether/Ethyl acetate=10/1 to 2/1) to give the title compound (420 mg, 58% yield) as a yellow solid. LC-MS (ESI+) m/z 669.3 (M+3H)⁺; ¹H NMR (400 MHz, DMSO-d₆) δ=10.80 (s, 1H), 7.63 (d, J=1.2 Hz, 1H), 7.51 (d, J=7.6 Hz, 1H), 7.28 (d, J=10.4 Hz, 1H), 7.18 (d, J=7.6 Hz, 1H), 6.91 (d, J=1.6 Hz, 1H), 6.59 (d, J=18.4 Hz, 2H), 4.23 (s, 2H), 3.80 (s, 3H), 3.65 (d, J=4.4 Hz, 4H), 3.46 (t, J=6.0 Hz, 2H), 3.23 (s, 4H), 2.55 (s, 4H), 2.25 (d, J=3.2 Hz, 4H), 1.47-1.42 (m, 10H), 1.17 (t, J=7.2 Hz, 1H), 1.07 (s, 3H).

### (1—(Cyclopropylmethyl)-7-(2-ethyl-6-methylpyridin-3-yl)-2-(1,2,5,6-tetrahydropyridin-3-yl)-1H-indol-5-yl)(4-(5-fluoro-3-methoxy-6-methylpyridin-2-yl)piperazin-1-yl)methanone (Intermediate RJ)

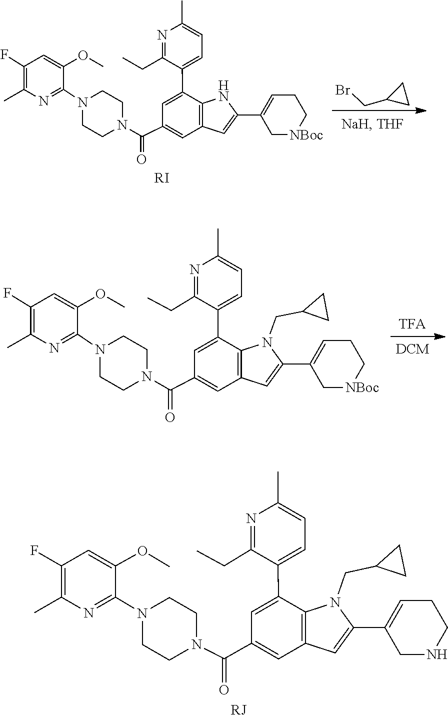

### Step 1—Tert-butyl 3-(1-(cyclopropylmethyl)-7-(2-ethyl-6-methylpyridin-3-yl)-5-(4-(5-fluoro-3-methoxy-6-methylpyridin-2-yl)piperazine-1-carbonyl)-1H-indol-2-yl)-5,6-dihydropyridine-1(2H)-carboxylate

To a solution of tert-butyl 5-[7-(2-ethyl-6-methyl-3-pyridyl)-5-[4-(5-fluoro-3-methoxy-6-methyl-2-pyridyl)piperazine-1-carbonyl]-1H-indol-2-yl]-3,6-dihydro-2H-pyridine-1-carboxylate (400 mg, 598 μmol, Intermediate RI) in THF (4 mL) was added NaH (35.8 mg, 897 μmol, 60% dispersion in mineral oil) at 0° C. for 0.5 hr. Then bromomethylcyclopropane (121 mg, 897 μmol, 85.6 μL) was added and the mixture was stirred at 25° C. for 11.5 hrs. On completion, the reaction mixture was quenched by addition of NH₄Cl (5 mL) at 0° C., and then diluted with H₂O (30 mL) and extracted with EA (30 mL×3). The combined organic layers were dried over Na₂SO₄, filtered and concentrated under reduced pressure to give a residue. The residue was purified by column chromatography (SiO₂, Petroleum ether/Ethyl acetate=4/1 to 0/1) to give the title compound (200 mg, 37% yield) as a white solid. LC-MS (ESI⁺) m/z 723.6 (M+H)⁺.

### Step 2—(1—(Cyclopropylmethyl)-7-(2-ethyl-6-methylpyridin-3-yl)-2-(1,2,5,6-tetrahydropyridin-3-yl)-1H-indol-5-yl)(4-(5-fluoro-3-methoxy-6-methylpyridin-2-yl)piperazin-1-yl)methanone

To a solution of tert-butyl 5-[1-(cyclopropylmethyl)-7-(2-ethyl-6-methyl-3-pyridyl)-5-[4-(5-fluoro-3-methoxy-6-methyl-2-pyridyl)piperazine-1-carbonyl]indol-2-yl]-3,6-dihydro-2H-pyridine-1-carboxylate (150 mg, 207 μmol) in TFA (1 mL). The mixture was then stirred at 25° C. for 1 hr. On completion, the reaction mixture was filtered and concentrated under reduced pressure to give the title compound (130 mg, 91% yield) as a white solid. LC-MS (ESI⁺) m/z 623.3 (M+H)⁺.

### 7-Bromo-2-(1-(tert-butoxycarbonyl)-1,2,5,6-tetrahydropyridin-3-yl)-1-ethyl-1H-indole-5-carboxylic acid (Intermediate RK)

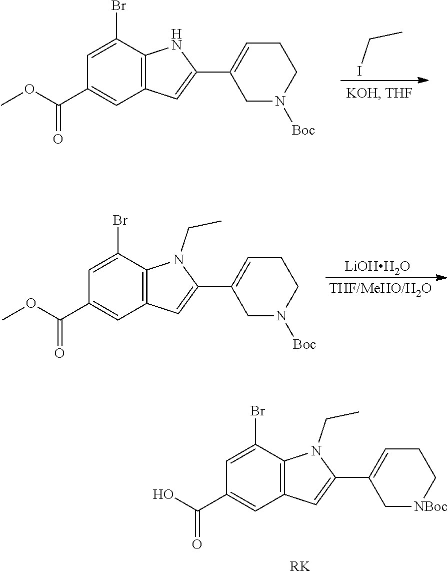

### Step 1—Methyl 7-bromo-2-(1-(tert-butoxycarbonyl)-1,2,5,6-tetrahydropyridin-3-yl)-1-ethyl-1H-indole-5-carboxylate

To a solution of methyl 7-bromo-2-(1-tert-butoxycarbonyl-3,6-dihydro-2H-pyridin-5-yl)-1H-indole-5-carboxylate (1 g, 2 mmol, synthesized via Steps 1-2 from Intermediate NM), iodoethane (1.07 g, 6.89 mmol, 551 μL) in THE (15 mL) was added KOH (193 mg, 3.45 mmol) at 0° C. The mixture was then stirred at 25° C. for 12 hrs. On completion, the reaction mixture was filtered and filtrate was concentrated under reduced pressure to give a residue. The residue was purified by column chromatography (SiO₂, Petroleum ether/Ethyl acetate=8/1 to 1/1) to give the title compound (900 mg, 65% yield) as a yellow solid. LC-MS (ESI+) m/z 407.0 (M−55)⁺; ¹H NMR (400 MHz, DMSO-d6) δ=8.22 (d, J=1.2 Hz, 1H), 7.89 (d, J=1.6 Hz, 1H), 6.68 (s, 1H), 6.12 (s, 1H), 4.54 (d, J=7.2 Hz, 2H), 4.07 (s, 2H), 3.86 (s, 3H), 3.53 (t, J=5.6 Hz, 2H), 2.31 (d, J=3.2 Hz, 2H), 1.43 (s, 9H), 1.24 (s, 3H).

### Step 2—7-Bromo-2-(1-(tert-butoxycarbonyl)-1,2,5,6-tetrahydropyridin-3-yl)-1-ethyl-1H-indole-5-carboxylic acid

To a solution of methyl 7-bromo-2-(1-tert-butoxycarbonyl-3,6-dihydro-2H-pyridin-5-yl)-1-ethyl-indole-5-carboxylate (900 mg, 1.94 mmol) in THF (4 mL), MeOH (1 mL), and H₂O (1 mL) was added LiOH·H₂O (407 mg, 9.71 mmol). The mixture was then stirred at 40° C. for 12 hrs. On completion, the reaction mixture was quenched by addition of 1M HCl (10 mL) at 0° C., and then diluted with H₂O (30 mL) and extracted with EA (30 mL×3). The combined organic layers were dried over Na₂SO₄, filtered and concentrated under reduced pressure to give the title compound (800 mg, 87% yield) as a white solid. LC-MS (ESI⁺) m/z 349.0 (M−99)⁺.

### (7-Bromo-1-ethyl-2-(1,2,5,6-tetrahydropyridin-3-yl)-1H-indol-5-yl)(4-(5-fluoro-3-methoxypyridin-2-yl)piperazin-1-yl)methanone (Intermediate RL)

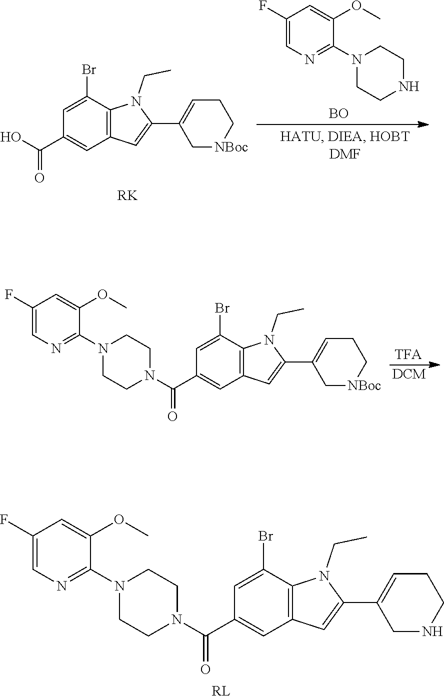

### Step 1—Tert-butyl 3-(7-bromo-1-ethyl-5-(4-(5-fluoro-3-methoxypyridin-2-yl)piperazine-1-carbonyl)-1H-indol-2-yl)-5,6-dihydropyridine-1(2H)-carboxylate

To a solution of 7-bromo-2-(1-tert-butoxycarbonyl-3,6-dihydro-2H-pyridin-5-yl)-1-ethyl-indole-5-carboxylic acid (400 mg, 890 μmol, Intermediate RK) in DMF (7 mL) was added HATU (406 mg, 1.07 mmol), HOBt (240 mg, 1.78 mmol), DIEA (460 mg, 3.56 mmol, 620 μL), and 1-(5-fluoro-3-methoxy-2-pyridyl)piperazine (225 mg, 1.07 mmol, Intermediate BO). The mixture was then stirred at 25° C. for 0.2 hr. On completion, the reaction mixture was quenched by addition of H₂O (10 mL), and then filtered and the filtrate was concentrated under reduced pressure to give a residue. The crude product was purified by re-crystallization from PE (50 mL) at 0° C. to the title compound (500 mg, 78% yield) as a yellow solid. LC-MS (ESI+) m/z 644.0 (M+H)⁺; ¹H NMR (400 MHz, DMSO-d6) δ=7.79 (d, J=2.4 Hz, 1H), 7.65 (d, J=1.2 Hz, 1H), 7.44-7.33 (m, 2H), 6.57 (s, 1H), 6.11 (s, 1H), 5.75 (s, 1H), 4.53 (d, J=7.2 Hz, 2H), 4.15-4.04 (m, 2H), 3.84 (s, 3H), 3.74-3.58 (m, 3H), 3.53 (t, J=5.2 Hz, 2H), 3.24 (s, 3H), 2.91-2.63 (m, 1H), 2.32 (d, J=2.0 Hz, 2H), 1.43 (s, 9H), 1.22 (t, J=6.8 Hz, 3H).

### Step 2—(7-Bromo-1-ethyl-2-(1,2,5,6-tetrahydropyridin-3-yl)-1H-indol-5-yl)(4-(5-fluoro-3-methoxypyridin-2-yl)piperazin-1-yl)methanone

A solution of tert-butyl 5-[7-bromo-1-ethyl-5-[4-(5-fluoro-3-methoxy-2-pyridyl) piperazine-1-carbonyl]indol-2-yl]-3, 6-dihydro-2H-pyridine-1-carboxylate (500 mg, 778 μmol) in TFA (4 mL) was stirred at 25° C. for 1 hr. On completion, the reaction mixture was concentrated under reduced pressure to give the title compound (420 mg, 81% yield, TFA) as a white solid. LC-MS (ESI⁺) m/z 542.2 (M+H)⁺.

### 1-(3-(7-Bromo-1-ethyl-5-(4-(5-fluoro-3-methoxypyridin-2-yl)piperazine-1-carbonyl)-1H-indol-2-yl)-5,6-dihydropyridin-1(2H)-yl)-3-(1H-1,2,3-triazol-1-yl)propan-1-one (Intermediate RM)

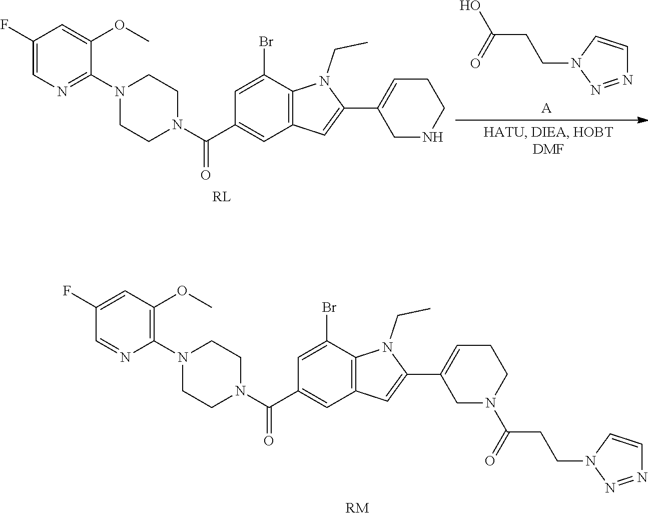

To a solution of [7-bromo-1-ethyl-2-(1,2,3,6-tetrahydropyridin-5-yl)indol-5-yl]-[4-(5-fluoro-3-methoxy-2-pyridyl)piperazin-1-yl]methanone (400 mg, 737 μmol, Intermediate RL) in DMF (5 mL) was added HATU (336 mg, 884 μmol), HOBt (199 mg, 1.47 mmol), DIEA (381 mg, 2.95 mmol, 513 μL), and 3-(triazol-1-yl)propanoic acid (124 mg, 884 μmol, Intermediate A). The mixture was then stirred at 25° C. for 0.2 hr. On completion, the reaction mixture was filtered and concentrated under reduced pressure to give a residue. The crude product was purified by reverse-phase HPLC (0.1% FA condition) to give the title compound (350 mg, 62% yield, FA) as a white solid. LC-MS (ESI⁺) m/z 665.2 (M+H)⁺; ¹H NMR (400 MHz, DMSO-d₆) δ=8.11 (d, J=7.2 Hz, 1H), 7.84-7.61 (m, 3H), 7.45-7.29 (m, 2H), 6.69-6.52 (m, 1H), 6.19-6.05 (m, 1H), 4.69-4.41 (m, 4H), 4.20 (d, J=7.6 Hz, 2H), 3.84 (s, 3H), 3.65 (td, J=5.6, 14.4 Hz, 5H), 3.24 (s, 4H), 3.10 (td, J=6.8, 10.0 Hz, 2H), 2.48-2.44 (m, 1H), 2.40-2.27 (m, 2H), 1.26-1.11 (m, 5H).

### 6-(1-(3-(1H-pyrazol-1-yl)propanoyl)-1,2,5,6-tetrahydropyridin-3-yl)-7-fluoro-4-(4,4,5,5-tetramethyl-1,3,2-dioxaborolan-2-yl)-1H-indole-2-carboxylic acid (Intermediate RN)

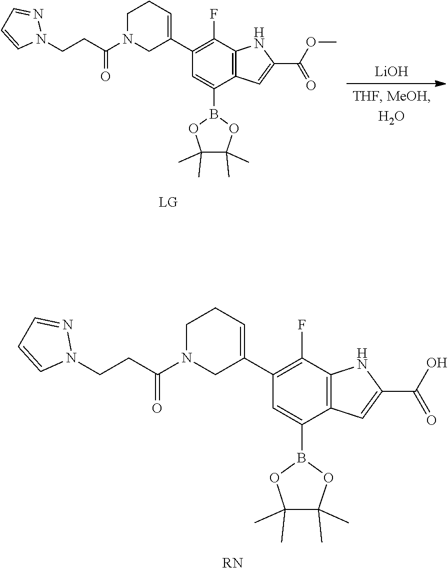 [SMILES: COC(=O)c1cc2c(B3OC(C)(C)C(C)(C)O3)cc(C3=CCCN(C(=O)CCn4cccn4)C3)c(F)c2[nH]1 ; "LiOH" ; "LG" ; CC1(C)OB(c2cc(C3=CCCN(C(=O)CCn4cccn4)C3)c(F)c3[nH]c(C(=O)O)cc23)OC1(C)C ; "RN"]

To a solution of methyl 7-fluoro-6-[1-(3-pyrazol-1-ylpropanoyl)-3,6-dihydro-2H-pyridin-5-yl]-4-(4,4,5,5-tetramethyl-1,3,2-dioxaborolan-2-yl)-1H-indole-2-carboxylate (500 mg, 957 μmol, Intermediate LG) in THF (3 mL), MeOH (1 mL) and H₂O (1 mL) was added LiOH·H₂O (160 mg, 3.83 mmol). The mixture was then stirred at 25° C. for 0.5 hr. Upon completion, the mixture was diluted with H₂O (10 mL) and extracted with EA (5 mL×3). The combined organic layers were dried over Na₂SO₄, filtered and concentrated under reduced pressure to give the title compound (370 mg) as a yellow gum. LC-MS (ESI⁺) m/z 509.2 (M+H)⁺.

### 1-(3-(7-Fluoro-2-(4-(5-fluoro-3-methoxy-6-methylpyridin-2-yl)piperazine-1-carbonyl)-4-(4,4,5,5-tetramethyl-1,3,2-dioxaborolan-2-yl)-1H-indol-6-yl)-5,6-dihydropyridin-1(2H)-yl)-3-(1H-pyrazol-1-yl)propan-1-one (Intermediate RO)

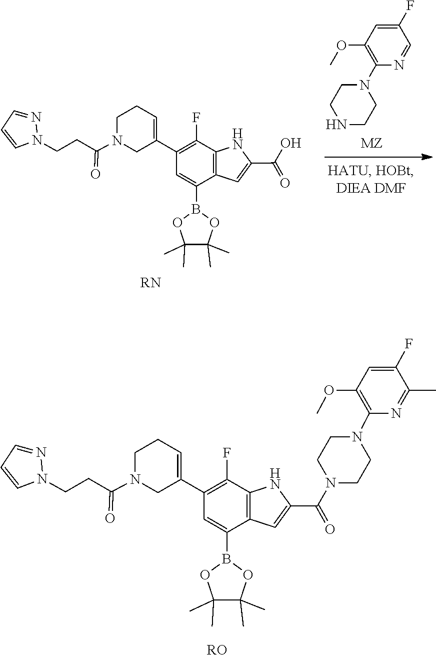

To a solution of 7-fluoro-6-[1-(3-pyrazol-1-ylpropanoyl)-3,6-dihydro-2H-pyridin-5-yl]-4-(4,4,5,5-tetramethyl-1,3,2-dioxaborolan-2-yl)-1H-indole-2-carboxylic acid (300 mg, 590 μmol, Intermediate RN) in DMF (5 mL) was added DIEA (305 mg, 2.36 mmol), 1-(5-fluoro-3-methoxy-6-methyl-2-pyridyl)piperazine (154 mg, 590 μmol, HCl, Intermediate MZ), HATU (269 mg, 708 μmol) and HOBt (159 mg, 1.18 mmol). The mixture was then stirred at 25° C. for 10 mins. Upon completion, the mixture was diluted with H₂O (20 mL) and extracted with EA (5 mL×3). The combined organic layers were washed with aqueous NaCl (5 mL×3), dried over Na₂SO₄, filtered and concentrated under reduced pressure to give a residue. The residue was purified by column chromatography (SiO₂, Petroleum ether/Ethyl acetate=50/1 to 0/1) to afford the title compound (300 mg, 56% yield) as a white solid. LC-MS (ESI⁺) m/z 716.4 (M+H)⁺; ¹H NMR (400 MHz, DMSO-d₆) δ=12.17-12.03 (m, 1H), 7.74-7.69 (m, 1H), 7.44-7.38 (m, 1H), 7.36-7.27 (m, 2H), 6.99 (s, 1H), 6.23-6.17 (m, 1H), 6.04 (s, 1H), 4.42-4.21 (m, 5H), 3.81 (s, 3H), 3.77 (s, 4H), 3.65-3.56 (m, 2H), 3.30 (s, 4H), 3.00-2.93 (m, 2H), 2.33 (s, 1H), 2.26 (d, J=2.8 Hz, 3H), 1.33 (s, 12H).

### 5-Chloro-2,4-diethylpyridine (Intermediate RP)

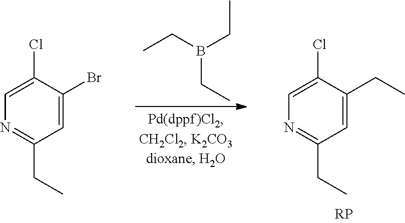

To a solution of 4-bromo-5-chloro-2-ethyl-pyridine (210 mg, 952 μmol, CAS #2770349-40-9) in dioxane (4 mL) and H₂O (0.2 mL) were added Et₃B (93.3 mg, 952 μmol), Pd(dppf)Cl₂ (69.6 mg, 95.2 μmol) and K₂CO₃ (263 mg, 1.90 mmol). The mixture was then stirred at 80° C. for 2 hrs. Upon completion, the mixture was concentrated under reduced pressure to give a residue. The residue was purified by flash silica gel chromatography (ISCO®; 4 g SepaFlash® Silica Flash Column, Eluent of 0˜30% Ethyl acetate/Petroleum ether gradient @40 mL/min) to afford the title compound (110 mg, 57% yield) as a colorless oil. LC-MS (ESI⁺) m/z 170.1 (M+H)⁺; ¹H NMR (400 MHz, CDCl₃-d) δ=8.41 (s, 1H), 7.03 (s, 1H), 2.82-2.69 (m, 4H), 1.27 (td, J=7.6, 18.4 Hz, 6H).

### 1-(3-(7-Fluoro-2-(4-(4-fluoro-2-methoxyphenyl)piperazine-1-carbonyl)-4-(4,4,5,5-tetramethyl-1,3,2-dioxaborolan-2-yl)-1H-indol-6-yl)-5,6-dihydropyridin-1(2H)-yl)-3-(1H-1,2,3-triazol-1-yl)propan-1-one (Intermediate RO)

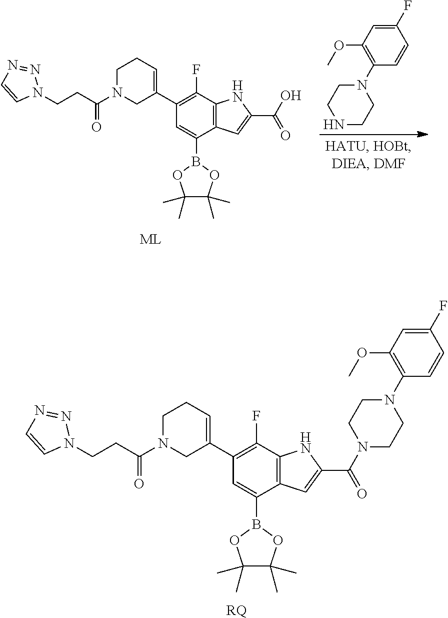

To a solution of 7-fluoro-4-(4,4,5,5-tetramethyl-1,3,2-dioxaborolan-2-yl)-6-[1-[3-(triazol-1-yl)propanoyl]-3,6-dihydro-2H-pyridin-5-yl]-1H-indole-2-carboxylic acid (150.00 mg, 265.05 μmol, Intermediate ML), 1-(4-fluoro-2-methoxy-phenyl)piperazine (98.09 mg, 397.58 μmol, HCl, CAS #186386-96-9), HOBt (71.63 mg, 530.10 μmol), and DIEA (205.53 mg, 1.59 mmol, 277.00 μL) in DMF (3 mL) was added HATU (151.17 mg, 397.58 μmol). The reaction mixture was stirred at 20° C. for 0.25 h. Upon completion, the mixture was poured into H₂O (10 mL) and the mixture was filtered to get the filter cake. The crude product was triturated with H₂O (6 mL) at 20° C. for 10 min to give the title compound (115.00 mg, 62% yield) as a white solid. LC-MS (ESI⁺)/z 702.4 (M+H)⁺.

### 1-Methyl-4,5,6,7-tetrahydropyrazolo[4,3-c]pyridine (CAS #100501-58-4) (Intermediate RR)

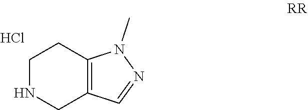

### 1-Methyl-4,5,6,7-tetrahydropyrazolo[3,4-c]pyridine (CAS #1228878-69-0) (Intermediate RS)

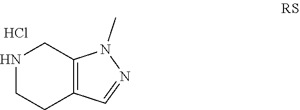

### Tert-butyl 3-(2-(1-ethyl-1,4,5,6-tetrahydropyrrolo[3,4-c]pyrazole-5-carbonyl)-7-fluoro-4-(4,4,5,5-tetramethyl-1,3,2-dioxaborolan-2-yl)-1H-indol-6-yl)-5,6-dihydropyridine-1(2H)-carboxylate (Intermediate RT)

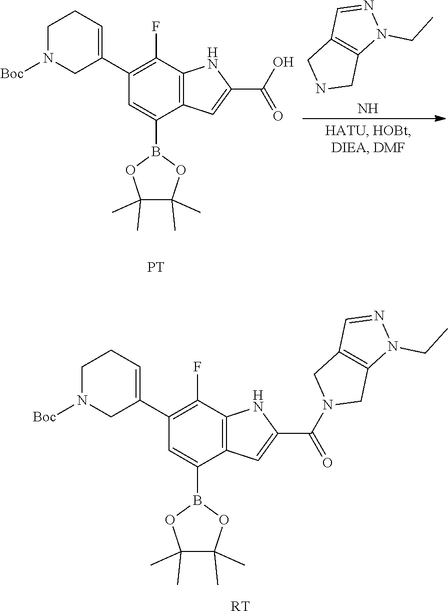

To a solution of 6-(1-tert-butoxycarbonyl-3,6-dihydro-2H-pyridin-5-yl)-7-fluoro-4-(4,4,5,5-tetramethyl-1,3,2-dioxaborolan-2-yl)-1H-indole-2-carboxylic acid (0.85 g, 1.40 mmol, Intermediate PT) and 1-ethyl-5,6-dihydro-4H-pyrrolo[3,4-c]pyrazole (291.34 mg, 1.68 mmol, HCl, Intermediate NH), HOBt (377.85 mg, 2.80 mmol), and DIEA (1.08 g, 8.39 mmol, 1.46 mL) in DMF (13 mL) was added HATU (637.97 mg, 1.68 mmol). The mixture was then stirred at 20° C. for 0.5 h. Upon completion, the mixture was filtered to give a residue. The crude product was triturated with EtOAc (10 mL×2) at 20° C. for 10 min to give the title compound (500.00 mg, 56% yield) as a white solid. LC-MS (ESI⁺) m/z 606.4 (M+H)⁺; ¹H NMR (400 MHz, CDCl₃-d) δ=9.54 (s, 1H), 7.57 (d, J=7.2 Hz, 1H), 7.49-7.34 (m, 2H), 6.12 (s, 1H), 5.26-4.97 (m, 2H), 4.96-4.76 (m, 2H), 4.35-4.13 (m, 4H), 3.60 (t, J=5.6 Hz, 2H), 2.35 (d, J=2.0 Hz, 2H), 1.56-1.47 (m, 12H), 1.45-1.37 (m, 12H).

### (4-(4-Ethylpyridin-3-yl)-7-fluoro-6-(1,2,5,6-tetrahydropyridin-3-yl)-1H-indol-2-yl)(1-ethylpyrrolo[3,4-c]pyrazol-5(1H,4H,6H)-yl)methanone (Intermediate RU)

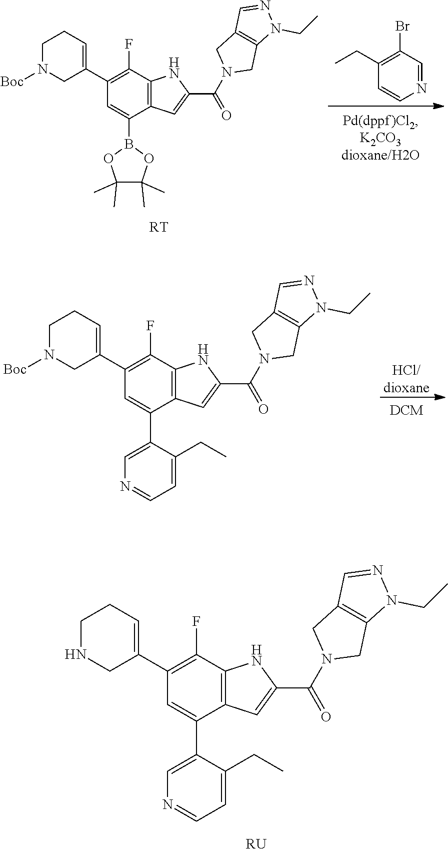

### Step 1—Tert-butyl 3-(2-(1-ethyl-1,4,5,6-tetrahydropyrrolo[3,4-c]pyrazole-5-carbonyl)-4-(4-ethylpyridin-3-yl)-7-fluoro-1H-indol-6-yl)-5,6-dihydropyridine-1(2H)-carboxylate

To a solution of tert-butyl 3-(2-(1-ethyl-1,4,5,6-tetrahydropyrrolo[3,4-c]pyrazole-5-carbonyl)-7-fluoro-4-(4,4,5,5-tetramethyl-1,3,2-dioxaborolan-2-yl)-1H-indol-6-yl)-5,6-dihydropyridine-1(2H)-carboxylate (250.00 mg, 412.88 mol, Intermediate RT) and 3-bromo-4-ethyl-pyridine (76.82 mg, 412.9 μmol, CAS #38749-76-7) in H₂O (0.6 mL) and dioxane (3 mL) were added K₂CO₃ (171.19 mg, 1.24 mmol) and Pd(dppf)Cl₂ (30.21 mg, 41.29 μmol). The mixture was then stirred at 80° C. for 1 h. Upon completion, the mixture was diluted with H₂O (2 mL) and extracted with EtOAc (5 mL×2). The combined organic layers were dried over Na₂SO₄, filtered and concentrated under reduced pressure to give a residue. The residue was purified by flash silica gel chromatography (ISCO®; 4 g SepaFlash® Silica Flash Column, Eluent of 0-100% EtOAc/Petroleum ether gradient @60 mL/min) to give the title compound (240.00 mg, 89% yield) as a brown solid. LC-MS (ESI⁺) m/z 585.3 (M+H)⁺.

### Step 2—(4-(4-Ethylpyridin-3-yl)-7-fluoro-6-(1,2,5,6-tetrahydropyridin-3-yl)-1H-indol-2-yl)(1-ethylpyrrolo[3,4-c]pyrazol-5(1H,4H,6H)-yl)methanone

To a solution of tert-butyl 3-(2-(1-ethyl-1,4,5,6-tetrahydropyrrolo[3,4-c]pyrazole-5-carbonyl)-4-(4-ethylpyridin-3-yl)-7-fluoro-1H-indol-6-yl)-5,6-dihydropyridine-1(2H)-carboxylate (240.00 mg, 410.48 μmol) in DCM (2 mL) was added HCl/dioxane (2 M, 4 mL). The mixture was then stirred at 20° C. for 1 h. Upon completion, the mixture was concentrated under reduced pressure to give the title compound (197.00 mg, 89% yield) as a white solid. LC-MS (ESI⁺) m/z 485.2 (M+H)⁺; ¹H NMR (400 MHz, CDCl₃-d) δ=9.86-9.50 (m, 1H), 8.60 (d, J=4.8 Hz, 1H), 8.57-8.50 (m, 1H), 7.38-7.31 (m, 1H), 7.29 (s, 1H), 6.91 (d, J=6.0 Hz, 1H), 6.65-6.50 (m, 1H), 6.16 (s, 1H), 4.91-4.75 (m, 4H), 4.22-4.08 (m, 2H), 3.78 (s, 1H), 3.08 (t, J=6.0 Hz, 2H), 2.57 (q, J=7.6 Hz, 2H), 2.32 (d, J=3.2 Hz, 2H), 1.53-1.46 (m, 3H), 1.15-1.05 (m, 3H).

### Ethyl 3-(4,4,5,5-tetramethyl-1,3,2-dioxaborolan-2-yl)cyclohex-3-ene-1-carboxylate (Intermediate RV)

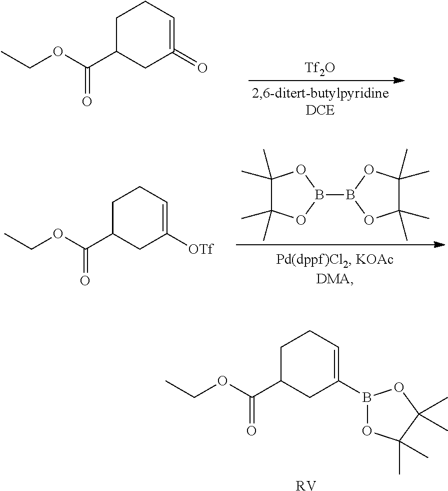

### Step 1—Ethyl 3-(trifluoromethylsulfonyloxy)cyclohex-3-ene-1-carboxylate

To a solution of ethyl 3-oxocyclohexanecarboxylate (1.1 g, 6.5 mmol, CAS #33668-25-6) and 2,6-ditert-butylpyridine (1.61 g, 8.40 mmol) in DCE (10 mL) was added Tf₂O (2.01 g, 7.11 mmol, 1.17 mL) dropwise under 0° C. The mixture was stirred at 0° C. for 0.5 h. Then the mixture was stirred at 20° C. for 1.5 h. Upon completion, the mixture was quenched by addition of H₂O (10 mL), and then extracted with EtOAc (10 mL×3). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 0˜9% EtOAc/Petroleum ether gradient @60 mL/min) to give the title compound (1.65 g, 85% yield) as a colorless oil. ¹H NMR (400 MHz, DMSO-d₆) δ=5.86-5.71 (m, 1H), 4.17 (q, J=7.2 Hz, 2H), 2.81-2.70 (m, 1H), 2.69-2.59 (m, 1H), 2.57-2.47 (m, 1H), 2.38-2.18 (m, 2H), 2.09-1.94 (m, 1H), 1.80-1.58 (m, 1H), 1.31-1.23 (m, 3H).

### Step 2—Ethyl 3-(4,4,5,5-tetramethyl-1,3,2-dioxaborolan-2-yl)cyclohex-3-ene-1-carboxylate

A mixture of ethyl 3-(trifluoromethylsulfonyloxy)cyclohex-3-ene-1-carboxylate (0.5 g, 1.7 mmol), 4,4,5,5-tetramethyl-2-(4,4,5,5-tetramethyl-1,3,2-dioxaborolan-2-yl)-1,3,2-dioxaborolane (504 mg, 1.99 mmol), KOAc (325 mg, 3.31 mmol), and Pd(dppf)Cl₂ (61 mg, 83 μmol) in DMA (10 mL) was degassed and purged with N₂ three times. Then the mixture was stirred at 90° C. for 12 h under N₂ atmosphere. Upon completion, the mixture was filtered and the filtrate was concentrated under reduced pressure to give the residue. The residue was purified by flash silica gel chromatography (ISCO®; 12 g SepaFlash® Silica Flash Column, Eluent of 0˜17% EtOAc/Petroleum ether gradient @60 mL/min) to give the title compound (310 mg, 67% yield) as a colorless oil. ¹H NMR (400 MHz, DMSO-d₆) δ=6.60-6.50 (m, 1H), 4.21-4.07 (m, 2H), 2.57-2.40 (m, 2H), 2.32-2.11 (m, 3H), 2.02-1.93 (m, 1H), 1.74-1.57 (m, 1H), 1.33-1.19 (m, 10H).

### Methyl 3-chloro-3-oxopropanoate (CAS #37517-81-0) (Intermediate R^{w})

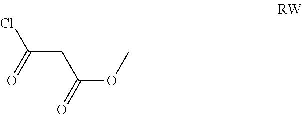

### Tert-butyl 3-(7-(2-methoxypyridin-3-yl)-5-(1-methyl-1,4,5,6-tetrahydropyrrolo[3,4-c]pyrazole-5-carbonyl)-1H-indol-2-yl)-5,6-dihydropyridine-1(2H)-carboxylate (Intermediate RX)

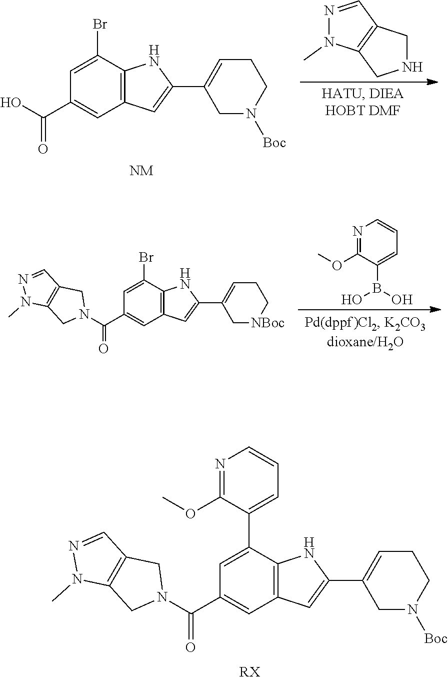

### Step 1—Tert-butyl 3-(7-bromo-5-(1-methyl-1,4,5,6-tetrahydropyrrolo[3,4-c]pyrazole-5-carbonyl)-1H-indol-2-yl)-5,6-dihydropyridine-1(2H)-carboxylate

To a solution of 7-bromo-2-(1-tert-butoxycarbonyl-3,6-dihydro-2H-pyridin-5-yl)-1H-indole-5-carboxylic acid (500 mg, 1.19 mmol, Intermediate NM) in DMF (7 mL) was added HATU (541 mg, 1.42 mmol), HOBt (320 mg, 2.37 mmol), DIEA (613 mg, 4.75 mmol, 826 μL,), and 1-methyl-5,6-dihydro-4H-pyrrolo[3,4-c]pyrazole (175 mg, 1.42 mmol, CAS #762233-62-5). The mixture was then stirred at 25° C. for 0.2 hr. On completion, the resulting solid was filtered, washed with H₂O (40 mL) and PE (40 mL) and dried under reduced pressure to give the title compound (550 mg, 84% yield) as a yellow solid. LC-MS (ESI+) m/z 527.9 (M+H)⁺; ¹H NMR (400 MHz, DMSO-d6) δ=11.28 (s, 1H), 7.81 (d, J=9.6 Hz, 1H), 7.49 (d, J=7.2 Hz, 1H), 7.30-7.09 (m, 1H), 6.83 (s, 1H), 6.61 (s, 1H), 4.72 (d, J=14.8 Hz, 2H), 4.57 (d, J=12.8 Hz, 2H), 4.26 (s, 2H), 3.80 (s, 1H), 3.68 (s, 1H), 3.50 (t, J=5.2 Hz, 2H), 2.90-2.72 (m, 1H), 2.32 (s, 2H), 1.44 (s, 9H).

### Step 2—Tert-butyl 3-(7-(2-methoxypyridin-3-yl)-5-(1-methyl-L4,5,6-tetrahydropyrrolo[3,4-c]pyrazole-5-carbonyl)-1H-indol-2-yl)-5,6-dihydropyridine-1(2H)-carboxylate

A mixture of tert-butyl 3-(7-bromo-5-(1-methyl-1,4,5,6-tetrahydropyrrolo[3,4-c]pyrazole-5-carbonyl)-1H-indol-2-yl)-5,6-dihydropyridine-1(2H)-carboxylate (0.6 g, 1.14 mmol), (2-methoxypyridin-3-yl)boronic acid (261 mg, 1.71 mmol, CAS #163105-90-6), Pd(dppf)Cl₂ (93.0 mg, 113 mol), and K₂CO₃ (475 mg, 3.42 mmol) in dioxane (4 mL) and H₂O (1 mL) was degassed and purged with N₂ three times. Then the mixture was stirred at 80° C. for 2 hrs under N₂ atmosphere. On completion, the reaction mixture was triturated with H₂O at 0° C. for 15 min to give the title compound (0.5 g) as a white solid. LC-MS (ESI⁺) m/z 555.5 (M+H)⁺.

### (1—(Cyclopropylmethyl)-7-(2-methoxypyridin-3-yl)-2-(1,2,5,6-tetrahydropyridin-3-yl)-1H-indol-5-yl)(1-methylpyrrolo[3,4-c]pyrazol-5(1H,4H,6H)-yl)methanone (Intermediate RY)

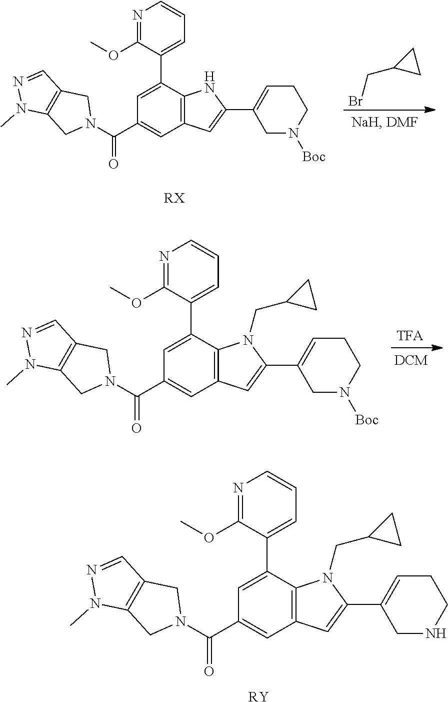

### Step 1—Tert-butyl 3-(1-(cyclopropylmethyl)-7-(2-methoxypyridin-3-yl)-5-(1-methyl-1,4,5,6-tetrahydropyrrolo[3,4-c]pyrazole-5-carbonyl)-1H-indol-2-yl)-5,6-dihydropyridine-1(2H′)-carboxylate

A mixture of tert-butyl 3-(7-(2-methoxypyridin-3-yl)-5-(1-methyl-1,4,5,6-tetrahydropyrrolo[3,4-c]pyrazole-5-carbonyl)-1H-indol-2-yl)-5,6-dihydropyridine-1(2H)-carboxylate (0.46 g, 829 μmol, Intermediate RX) in DMF (5 mL) at 0° C. added NaH (49.7 mg, 1.24 mmol, 60% dispersion in mineral oil) for 1 hr under N₂ atmosphere. Then bromomethylcyclopropane (167 mg, 1.24 mmol, 118 μL) was added and the mixture was stirred at 25° C. for 11 hrs under N₂ atmosphere. On completion, the reaction mixture was triturated with aqueous NH₄Cl at 0° C. for 15 min to give the title compound (0.46 g) as a white solid. LC-MS (ESI⁺) m/z 609.3 (M+H)⁺.

### Step 2—(1—(Cyclopropylmethyl)-7-(2-methoxypyridin-3-yl)-2-(1,2,5,6-tetrahydropyridin-3-yl)-1H-indol-5-yl)(1-methylpyrrolo[3,4-c]pyrazol-5(1H,4H,6H)-yl)methanone

To a solution of tert-butyl 3-(1-(cyclopropylmethyl)-7-(2-methoxypyridin-3-yl)-5-(1-methyl-1,4,5,6-tetrahydropyrrolo[3,4-c]pyrazole-5-carbonyl)-1H-indol-2-yl)-5,6-dihydropyridine-1(2H)-carboxylate (0.46 g, 755 μmol) in DCM (5 mL) was added TFA (86.1 mg, 755 μmol, 56.1 μL). The mixture was then stirred at 25° C. for 2 hrs. On completion, the reaction mixture was filtered and concentrated under reduced pressure to give a residue to give the title compound (0.35 g, TFA) as a red solid. LC-MS (ESI⁺) m/z 509.3 (M+H)⁺.

### Tert-butyl 3-(7-(4-ethyl-6-methylpyridin-3-yl)-5-(4-(5-fluoro-3-methoxypyridin-2-yl)piperazine-1-carbonyl)-1H-indol-2-yl)-5,6-dihydropyridine-1(2H)-carboxylate (Intermediate RZ)

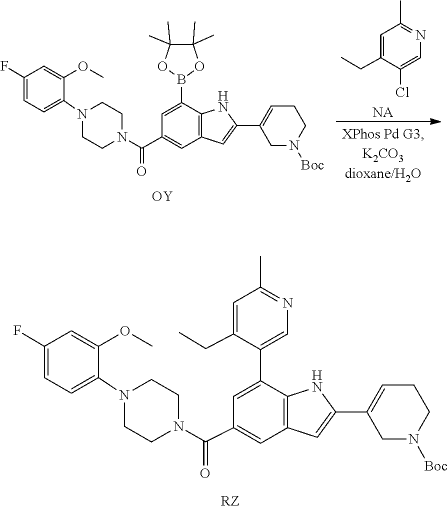

A mixture of tert-butyl 3-(5-(4-(5-fluoro-3-methoxypyridin-2-yl)piperazine-1-carbonyl)-7-(4,4,5,5-tetramethyl-1,3,2-dioxaborolan-2-yl)-1H-indol-2-yl)-5,6-dihydropyridine-1(2H)-carboxylate (0.3 g, 450 μmol, Intermediate OY), 5-chloro-4-ethyl-2-methylpyridine (122 mg, 589 μmol, Intermediate NA), XPhos Pd G3 (38.3 mg, 45.3 μmol), and K₃PO₄ (288 mg, 1.36 mmol) in dioxane (4 mL) and H₂O (1 mL) was degassed and purged with N₂ three times. Then the mixture was stirred at 80° C. for 2 hrs under N₂ atmosphere. On completion, the reaction mixture was filtered and concentrated under reduced pressure to give a residue. The crude residue was purified by column chromatography (SiO₂, Petroleum ether/Ethyl acetate=1/0 to 1/1) to give the title compound (0.14 g, 35% yield) as a white solid. LC-MS (ESI⁺) m/z 655.5 (M+H)⁺.

### (1—(Cyclopropylmethyl)-7-(4-ethyl-6-methylpyridin-3-yl)-2-(1,2,5,6-tetrahydropyridin-3-yl)-1H-indol-5-yl)(4-(5-fluoro-3-methoxypyridin-2-yl)piperazin-1-yl)methanone (Intermediate SA)

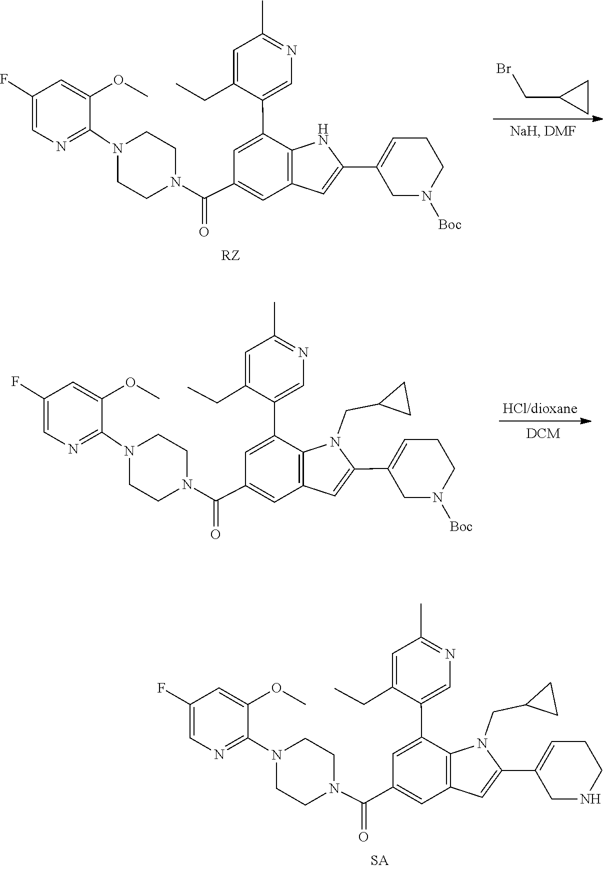 [SMILES: CCc1cc(C)ncc1-c1cc(C(=O)N2CCN(c3ncc(F)cc3OC)CC2)cc2cc(C3=CCCN(C(=O)OC(C)(C)C)C3)[nH]c12 ; BrCC1CC1 ; CCc1cc(C)ncc1-c1cc(C(=O)N2CCN(c3ncc(F)cc3OC)CC2)cc2cc(C3=CCCN(C(=O)OC(C)(C)C)C3)n(CC3CC3)c12 ; CCc1cc(C)ncc1-c1cc(C(=O)N2CCN(c3ncc(F)cc3OC)CC2)cc2cc(C3=CCCNC3)n(CC3CC3)c12]

### Step 1—Tert-butyl 3-(1-(cyclopropylmethyl)-7-(4-ethyl-6-methylpyridin-3-yl)-5-(4-(5-fluoro-3-methoxypyridin-2-yl)piperazine-1-carbonyl)-1H-indol-2-yl)-5,6-dihydropyridine-1(2H)-carboxylate

A mixture of tert-butyl 3-(7-(4-ethyl-6-methylpyridin-3-yl)-5-(4-(5-fluoro-3-methoxypyridin-2-yl)piperazine-1-carbonyl)-1H-indol-2-yl)-5,6-dihydropyridine-1(2H)-carboxylate (0.13 g, 198 μmol, Intermediate RZ) in DMF (2 mL) at 0° C. was added NaH (11.9 mg, 297 μmol, 60% dispersion in mineral oil) at 0° C. for 1 hr under N₂ atmosphere. Then bromomethylcyclopropane (53.6 mg, 397 μmol, 37.9 μL) was added and the mixture was stirred at 25° C. for 11 hrs under N₂ atmosphere. On completion, the reaction mixture was triturated with aqueous NH₄Cl at 0° C. for 15 min to give the title compound (0.12 g) as a white solid. LC-MS (ESI⁺) m/z 709.6 (M+H)⁺.

### Step 2—(1—(Cyclopropylmethyl)-7-(4-ethyl-6-methylpyridin-3-yl)-2-(1,2,5,6-tetrahydropyridin-3-yl)-1H-indol-5-yl)(4-(5-fluoro-3-methoxypyridin-2-yl)piperazin-1-yl)methanone

To a solution of tert-butyl 3-(1-(cyclopropylmethyl)-7-(4-ethyl-6-methylpyridin-3-yl)-5-(4-(5-fluoro-3-methoxypyridin-2-yl)piperazine-1-carbonyl)-1H-indol-2-yl)-5,6-dihydropyridine-1(2H)-carboxylate (0.12 g, 169 μmol) in DCM (2 mL) was added TFA (19.3 mg, 169 μmol, 12.57 μL). The mixture was then stirred at 25° C. for 2 hrs. On completion, the reaction mixture was filtered and concentrated under reduced pressure to give the title compound (0.1 g, TFA) as a red solid. LC-MS (ESI⁺) m/z 609.3 (M+H)⁺.

### 3-(Thiazol-2-yl)propanoic acid (CAS #14163-65-5) (Intermediate SB)

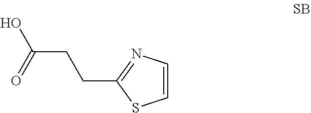

### 3-(1H-pyrazol-1-yl)propanoic acid (CAS #144163-65-5) (Intermediate SC)

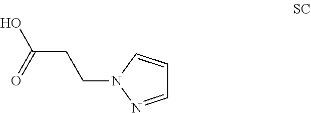

### Tert-butyl3-(7-bromo-5-(4-(4-fluoro-2-methoxyphenyl)piperazine-1-carbonyl)-1H-indol-2-yl)-5,6-dihydropyridine-1(2H)-carboxylate (Intermediate SD)

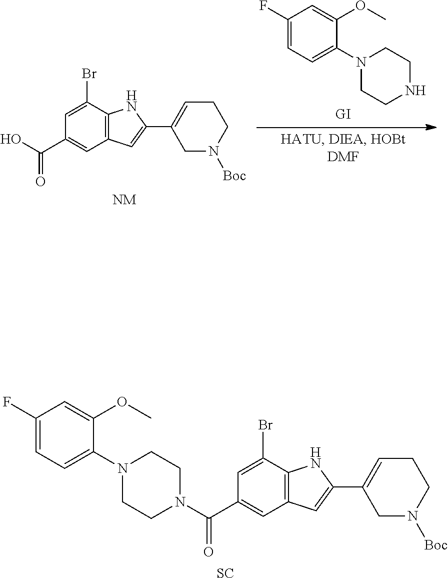

To a solution of 7-bromo-2-(1-(tert-butoxycarbonyl)-1,2,5,6-tetrahydropyridin-3-yl)-1H-indole-5-carboxylic acid (500 mg, 1.19 mmol, Intermediate NM) in DMF (2 mL) was added HATU (586 mg, 1.54 mmol), HOBt (240 mg, 1.78 mmol), DIEA (613 mg, 4.75 mmol) and 1-(4-fluoro-2-methoxyphenyl)piperazine (374 mg, 1.78 mmol, CAS #102392-11-0, Intermediate GI). Then the reaction was stirred at 25° C. for 10 min. On completion, the reaction mixture was quenched with water (20 mL) and extracted with ethyl acetate (3×20 mL). The combined organic layers were washed with brine (30 mL) and dried over anhydrous sodium sulfate, filtered and the filtrate was concentrated in vacuo to give the title compound (780 mg) as a white solid. LC-MS (ESI⁺) m/z 613.2 (M+H)⁺.

### Tert-butyl 3-(5-(4-(4-fluoro-2-methoxyphenyl)piperazine-1-carbonyl)-7-(4,4,5,5-tetramethyl-1,3,2-dioxaborolan-2-yl)-1H-indol-2-yl)-5,6-dihydropyridine-1(2H)-carboxylate (Intermediate SE)

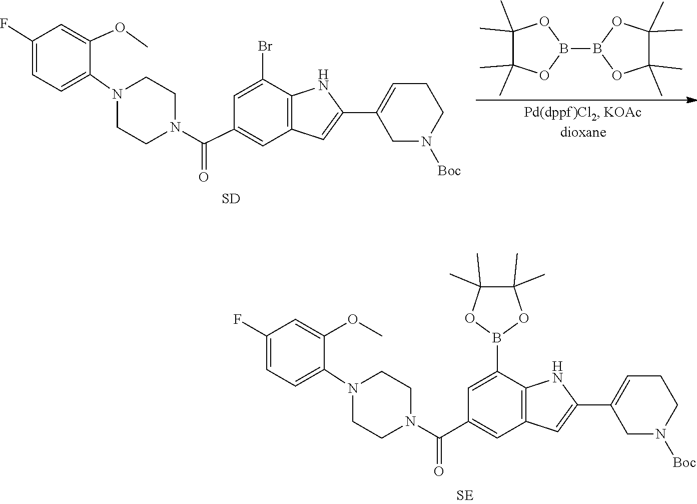

A mixture of tert-butyl 3-(7-bromo-5-(4-(4-fluoro-2-methoxyphenyl)piperazine-1-carbonyl)-1H-indol-2-yl)-5,6-dihydropyridine-1(2H)-carboxylate (450 mg, 733 μmol, Intermediate SD), 4,4,4′,4′,5,5,5′,5′-octamethyl-2,2′-bi(1,3,2-dioxaborolane) (186 mg, 733 μmol), KOAc (215 mg, 2.20 mmol), and Pd(dppf)Cl₂·CH₂Cl₂ (59.9 mg, 73.3 μmol) in dioxane (5 mL) was degassed and purged with N₂ three times. Then the mixture was stirred at 80° C. for 2 hrs under N₂ atmosphere. On completion, the reaction mixture was quenched with water (20 mL) and extracted with ethyl acetate (3×20 mL). The combined organic layers were washed withy brine (20 mL) and dried over anhydrous sodium sulfate, filtered and the filtrate was concentrated in vacuo to give the crude residue. The crude residue was purified by reversed-phase HPLC (0.8 g/L ammonium bicarbonate) to give the title compound (300 mg) as a yellow solid. ¹H NMR (400 MHz, DMSO-d₆) δ=9.65 (s, 1H), 7.76 (s, 1H), 7.48 (d, J=1.2 Hz, 1H), 6.91-6.84 (m, 2H), 6.68 (dt, J=2.8, 8.4 Hz, 1H), 6.57 (d, J=1.2 Hz, 1H), 6.44 (s, 1H), 4.33-4.20 (m, 2H), 3.81-3.78 (m, 3H), 3.66 (s, 3H), 3.51 (d, J=5.2 Hz, 2H), 3.28 (d, J=6.0 Hz, 2H), 2.91 (s, 4H), 2.47-2.44 (m, 2H), 2.33 (s, 2H), 1.45 (s, 9H), 1.38 (s, 12H).

### Tert-butyl 3-(7-(2-ethyl-6-methylpyridin-3-yl)-5-(4-(4-fluoro-2-methoxyphenyl)piperazine-1-carbonyl)-1H-indol-2-yl)-5,6-dihydropyridine-1(2H)-carboxylate (Intermediate SF)

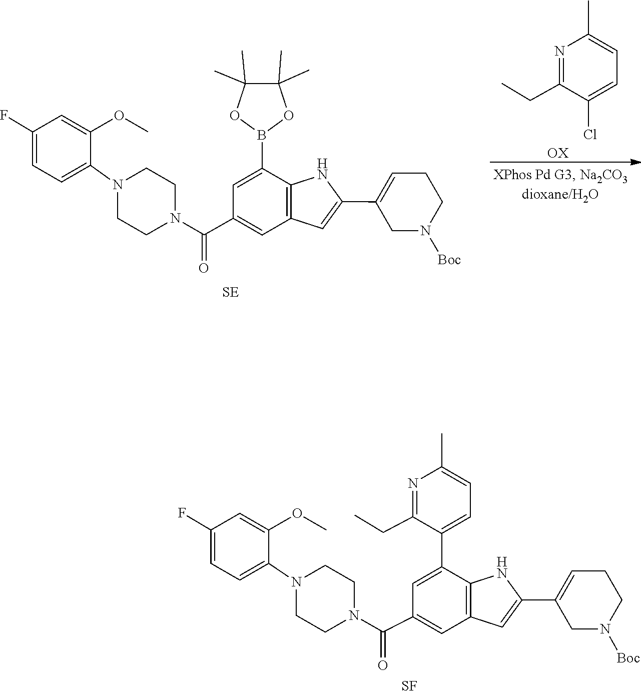

A mixture of 3-chloro-2-ethyl-6-methylpyridine (40.0 mg, 257 μmol, Intermediate OX), tert-butyl 3-(5-(4-(4-fluoro-2-methoxyphenyl)piperazine-1-carbonyl)-7-(4,4,5,5-tetramethyl-1,3,2-dioxaborolan-2-yl)-1H-indol-2-yl)-5,6-dihydropyridine-1(2H)-carboxylate (169 mg, 257 μmol, Intermediate SE), XPhos Pd G3 (21.7 mg, 25.7 μmol), and K₃PO₄ (163 mg, 771 μmol) in dioxane (1 mL) and H₂O (0.1 mL) was degassed and purged with N₂ three times. Then the mixture was stirred at 80° C. for 2 hrs under N₂ atmosphere. On completion, the reaction mixture was quenched with water (20 mL) and extracted with ethyl acetate (3×20 mL). The combined organic layers were washed with brine (20 mL) and dried over anhydrous sodium sulfate, filtered and the filtrate was concentrated in vacuo to give the title compound (130 mg) as a yellow solid. LC-MS (ESI⁺) m/z 654.3 (M+H)⁺.

### (1—(Cyclopropylmethyl)-7-(2-ethyl-6-methylpyridin-3-yl)-2-(1,2,5,6-tetrahydropyridin-3-yl)-1H-indol-5-yl)(4-(4-fluoro-2-methoxyphenyl)piperazin-1-yl)methanone (Intermediate SG)

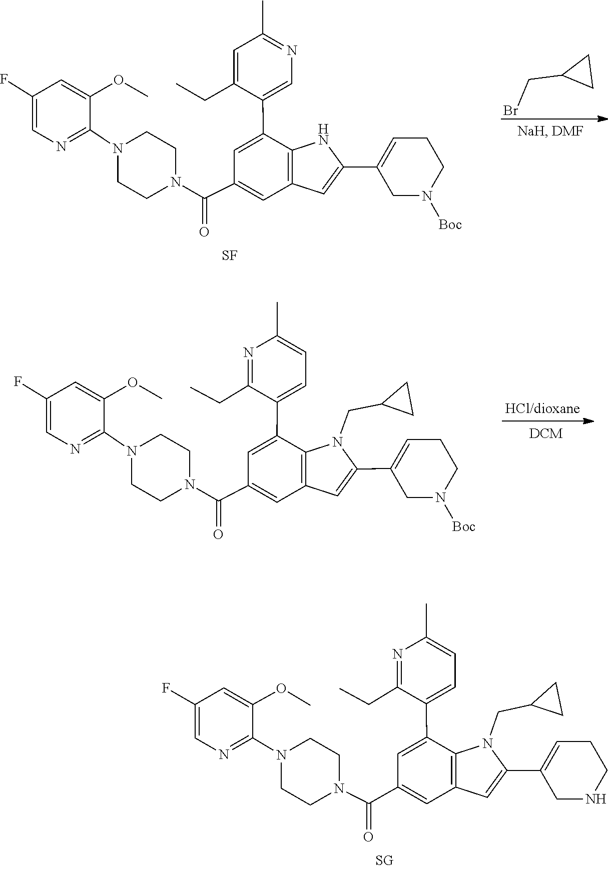

### Step 1—Tert-butyl 3-(1-(cyclopropylmethyl)-7-(2-ethyl-6-methylpyridin-3-yl)-5-(4-(4-fluoro-2-methoxyphenyl)piperazine-1-carbonyl)-1H-indol-2-yl)-5,6-dihydropyridine-1(2H)-carboxylate

To a solution of tert-butyl 3-(7-(2-ethyl-6-methylpyridin-3-yl)-5-(4-(4-fluoro-2-methoxyphenyl)piperazine-1-carbonyl)-1H-indol-2-yl)-5,6-dihydropyridine-1(2H)-carboxylate (130 mg, 198 μmol, Intermediate SF) in DMF (2 mL) was added NaH (11.9 mg, 298 μmol, 60% dispersion in mineral oil) and (bromomethyl)cyclopropane (32.2 mg, 238 μmol). The mixture was stirred at 0° C. for 2 hrs under N₂. On completion, the crude residue was purified by reverse-phase HPLC (0.8 g/L ammonium bicarbonate) to give the title compound (130 mg) as a yellow solid. ¹H NMR (400 MHz, DMSO-d₆) δ=7.72-7.64 (m, 2H), 7.25-7.19 (m, 1H), 6.93-6.81 (m, 3H), 6.71-6.61 (m, 2H), 6.01-5.94 (m, 1H), 3.79 (s, 3H), 3.72-3.57 (m, 4H), 2.95-2.89 (m, 4H), 2.46-2.40 (m, 3H), 2.26 (d, J=2.0 Hz, 2H), 1.41 (s, 9H), 1.23 (s, 2H), 1.08-0.99 (m, 4H), 0.16-0.06 (m, 3H), −0.19-−0.27 (m, 2H).

### Step 2—(1—(Cyclopropylmethyl)-7-(2-ethyl-6-methylpyridin-3-yl)-2-(1,2,5,6-tetrahydropyridin-3-yl)-1H-indol-5-yl)(4-(4-fluoro-2-methoxyphenyl)piperazin-1-yl)methanone

A mixture of tert-butyl 3-(1-(cyclopropylmethyl)-7-(2-ethyl-6-methylpyridin-3-yl)-5-(4-(4-fluoro-2-methoxyphenyl)piperazine-1-carbonyl)-1H-indol-2-yl)-5,6-dihydropyridine-1(2H)-carboxylate (80.0 mg, 113 μmol) in HCl (2 mL) and DCM (2 mL) was degassed and purged with N₂ three times. Then the mixture was stirred at 25° C. for 2 hrs. On completion, the mixture was concentrated in vacuo to give the title compound (50.0 mg) as a yellow solid. LC-MS (ESI⁺) m/z 608.4 (M+H)⁺.

### Tert-butyl 3-(7-bromo-5-(1-ethyl-1,4,5,6-tetrahydropyrrolo[3,4-c]pyrazole-5-carbonyl)-1H-indol-2-yl)-5,6-dihydropyridine-1(2H)-carboxylate (Intermediate SH)

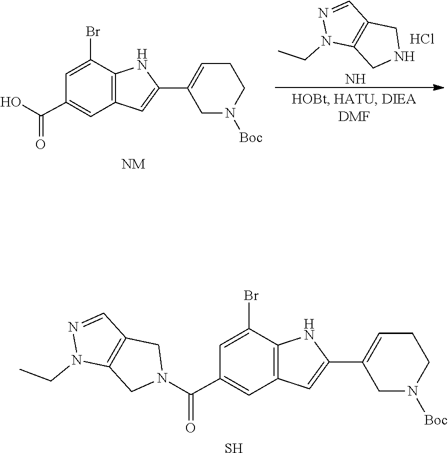

A mixture of 7-bromo-2-(1-(tert-butoxycarbonyl)-1,2,5,6-tetrahydropyridin-3-yl)-1H-indole-5-carboxylic acid (1.00 g, 2.37 mmol, Intermediate NM), 1-ethyl-1,4,5,6-tetrahydropyrrolo[3,4-c]pyrazole hydrochloride (618 mg, 3.56 mmol, HCl, Intermediate NH), HATU (1.08 g, 2.85 mmol), DIEA (1.84 g, 14.2 mmol) and HOBt (481 mg, 3.56 mmol) in DMF (10 mL) was stirred at 25° C. for 2 hrs. On completion, the crude product was triturated with water at 25° C. for 2 hrs, then the mixture was filtered and the filter cake was dried in vacuo to give the title compound (1.05 g) as a yellow solid. ¹H NMR (400 MHz, DMSO-d₆) δ=11.27 (s, 1H), 7.82 (s, 1H), 7.50 (d, J=2.8 Hz, 1H), 7.32-7.14 (m, 1H), 6.83 (s, 1H), 6.62 (s, 1H), 4.75 (d, J=13.2 Hz, 2H), 4.56 (d, J=14.4 Hz, 2H), 4.26 (s, 2H), 4.11 (q, J=7.2 Hz, 1H), 4.01-3.93 (m, 1H), 3.50 (t, J=5.2 Hz, 2H), 2.31 (d, J=2.0 Hz, 2H), 1.44 (s, 9H), 1.36 (s, 1H), 1.27-1.22 (m, 2H).

### Tert-butyl 3-(5-(1-ethyl-1,4,5,6-tetrahydropyrrolo[3,4-c]pyrazole-5-carbonyl)-7-(2-methoxyphenyl)-1H-indol-2-yl)-5,6-dihydropyridine-1(2H)-carboxylate (Intermediate SI)

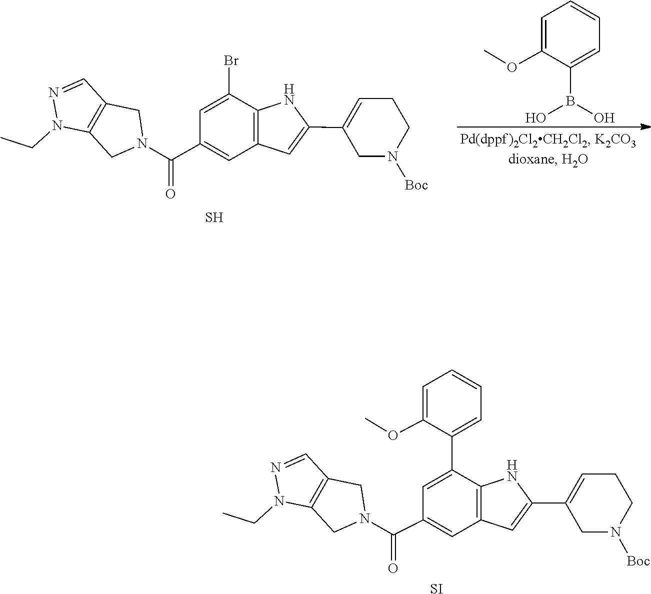

A mixture of tert-butyl 3-(7-bromo-5-(1-ethyl-1,4,5,6-tetrahydropyrrolo[3,4-c]pyrazole-5-carbonyl)-1H-indol-2-yl)-5,6-dihydropyridine-1(2H)-carboxylate (300 mg, 555 μmol, Intermediate SH), (2-methoxyphenyl)boronic acid (101 mg, 666 μmol, CAS #5720-06-9), K₂CO₃ (230 mg, 1.67 mmol), Pd(dppf)Cl₂·CH₂Cl₂ (45.3 mg, 55.5 μmol) in dioxane (3 mL) and H₂O (1 mL) was degassed and purged with N₂ three times. Then the mixture was stirred at 80° C. for 2 hrs under N₂ atmosphere. On completion, the crude product was triturated with water at 25° C. for 1 hr, then the mixture was filtered and the filter cake was dried in vacuo to give the title compound (240 mg) as a yellow solid. LC-MS (ESI⁺) m/z 568.3 (M+H)⁺.

### (1—(Cyclopropylmethyl)-7-(2-methoxyphenyl)-2-(1,2,5,6-tetrahydropyridin-3-yl)-1H-indol-5-yl)(1-ethylpyrrolo[3,4-c]pyrazol-5(1H,4H,6H)-yl)methanone (Intermediate SJ)

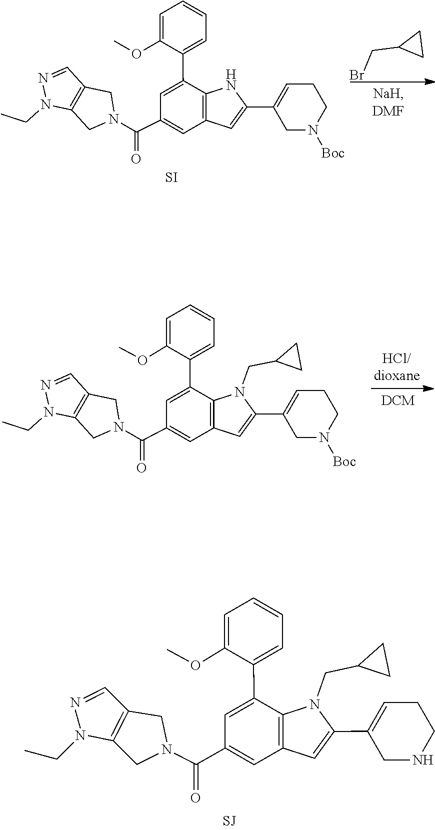

### Step 1—Tert-butyl 3-(1-(cyclopropylmethyl)-5-(1-ethyl-1,4,5,6-tetrahydropyrrolo[3,4-c]pyrazole-5-carbonyl)-7-(2-methoxyphenyl)-1H-indol-2-yl)-5,6-dihydropyridine-1(2H)-carboxylate

To a solution of tert-butyl 3-(5-(1-ethyl-1,4,5,6-tetrahydropyrrolo[3,4-c]pyrazole-5-carbonyl)-7-(2-methoxyphenyl)-1H-indol-2-yl)-5,6-dihydropyridine-1(2H)-carboxylate (150 mg, 264 μmol, Intermediate SI) in DMF (2 mL) was added NaH (15.8 mg, 396 μmol, 60% dispersion in mineral oil) and (bromomethyl)cyclopropane (53.5 mg, 396 μmol, CAS #7051-34-5). The mixture was stirred at 0° C. for 2 hrs. On completion, the reaction mixture was purified by reverse-phase HPLC (0.8 g/L ammonium bicarbonate) to give the title compound (160 mg) as a yellow solid. LC-MS (ESI⁺) m/z 622.3 (M+H)⁺

### Step 2—(1—(Cyclopropylmethyl)-7-(2-methoxyphenyl)-2-(1,2,5,6-tetrahydropyridin-3-yl)-1H-indol-5-yl)(1-ethylpyrrolo[3,4-c]pyrazol-5(1H,4H,6H)-yl)methanone

A mixture of tert-butyl 3-(1-(cyclopropylmethyl)-5-(1-ethyl-1,4,5,6-tetrahydropyrrolo[3,4-c]pyrazole-5-carbonyl)-7-(2-methoxyphenyl)-1H-indol-2-yl)-5,6-dihydropyridine-1(2H)-carboxylate (160 mg, 257 μmol) in DCM (2 mL) and HCl (1 mL) was stirred at 25° C. for 2 hrs. On completion, the mixture was concentrated in vacuo to give the title compound (120 mg) as a yellow solid. ¹H NMR (400 MHz, DMSO-d₆) δ=9.57-9.40 (m, 2H), 7.86-7.78 (m, 1H), 7.49-7.44 (m, 1H), 7.37-7.23 (m, 2H), 7.20-7.12 (m, 2H), 7.12-7.00 (m, 3H), 6.67 (s, 1H), 6.12-6.04 (m, 1H), 4.78 (d, J=11.6 Hz, 2H), 4.61-4.56 (m, 2H), 3.84 (d, J=16.4 Hz, 2H), 3.71 (s, 3H), 3.64 (dd, J=6.8, 15.2 Hz, 2H), 3.24 (d, J=4.0 Hz, 2H), 1.35-1.25 (m, 3H), 0.66-0.57 (m, 1H), 0.09-0.00 (m, 2H), −0.16-−0.27 (m, 2H).

### Tert-butyl 3-(5-(1-ethyl-1,4,5,6-tetrahydropyrrolo[3,4-c]pyrazole-5-carbonyl)-7-(4,4,5,5-tetramethyl-1,3,2-dioxaborolan-2-yl)-1H-indol-2-yl)-5,6-dihydropyridine-1(2H)-carboxylate (Intermediate SK)

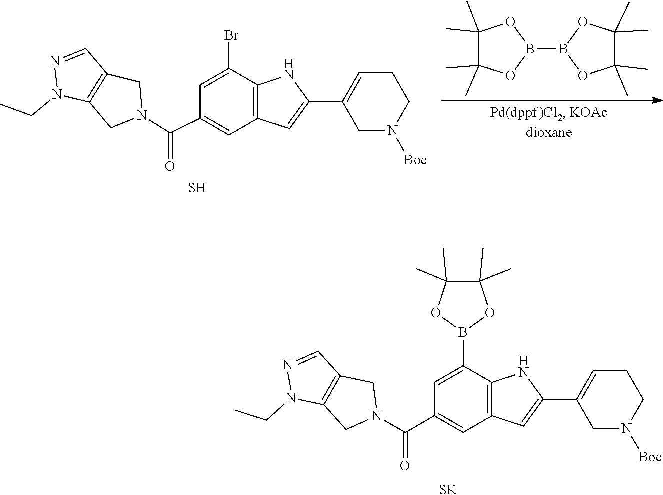

A mixture of tert-butyl 3-(7-bromo-5-(1-ethyl-1,4,5,6-tetrahydropyrrolo[3,4-c]pyrazole-5-carbonyl)-1H-indol-2-yl)-5,6-dihydropyridine-1(2H)-carboxylate (600 mg, 1.11 mmol, Intermediate SH), 4,4,4′,4′,5,5,5′,5′-octamethyl-2,2′-bi(1,3,2-dioxaborolane) (845 mg, 3.33 mmol), KOAc (326 mg, 3.33 mmol), and Pd(dppf)Cl₂·CH₂Cl₂ (90.6 mg, 111 μmol) in dioxane (6 mL) and was degassed and purged with N₂ three times. Then the mixture was stirred at 80° C. for 2 hrs under N₂ atmosphere. On completion, the reaction mixture was quenched with 10% sodium sulfite solution (20 mL) and extracted with dichloromethane (3×10 mL). The combined organic layers were washed with brine (25 mL) and dried over anhydrous sodium sulfate, filtered and the filtrate was concentrated in vacuo to give the residue. The crude residue was purified by reversed-phase HPLC (0.8 g/L ammonium bicarbonate) to give the title compound (260 mg) as a yellow solid. ¹H NMR (400 MHz, DMSO-d₆) δ=9.75-9.58 (m, 1H), 7.99-7.92 (m, 1H), 7.61 (d, J=6.8 Hz, 1H), 7.32-7.14 (m, 1H), 6.58 (s, 1H), 6.46 (s, 1H), 4.80-4.67 (m, 2H), 4.61-4.48 (m, 2H), 4.28 (s, 2H), 4.15-3.96 (m, 2H), 3.52 (s, 2H), 2.34 (s, 2H), 1.45 (s, 9H), 1.38 (s, 13H), 1.24 (t, J=7.2 Hz, 2H), 1.16 (s, 3H).

### Tert-butyl 3-(5-(1-ethyl-1,4,5,6-tetrahydropyrrolo[3,4-c]pyrazole-5-carbonyl)-7-(2-ethyl-6-methylpyridin-3-yl)-1H-indol-2-yl)-5,6-dihydropyridine-1(2H)-carboxylate (Intermediate SL)

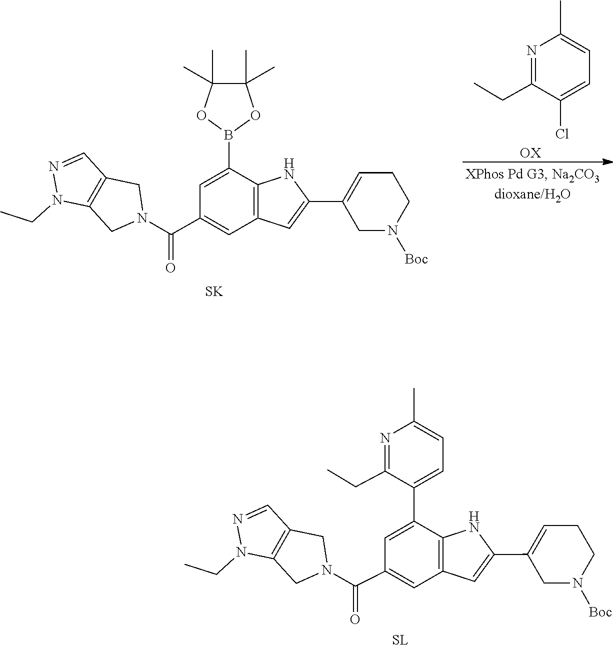

A mixture of 3-chloro-2-ethyl-6-methylpyridine (200 mg, 1.18 mmol, Intermediate OX), tert-butyl 3-(5-(1-ethyl-1,4,5,6-tetrahydropyrrolo[3,4-c]pyrazole-5-carbonyl)-7-(4,4,5,5-tetramethyl-1,3,2-dioxaborolan-2-yl)-1H-indol-2-yl)-5,6-dihydropyridine-1(2H)-carboxylate (678 mg, 1.18 mmol, Intermediate SK), K₃PO₄ (720 mg, 3.48 mmol), and XPhos Pd G3 (97.8 mg, 118 μmol) in dioxane (2 mL) and H₂O (0.4 mL) was degassed and purged with N₂ three times. Then the mixture was stirred at 80° C. for 2 hrs under N₂ atmosphere. On completion, the reaction mixture was quenched with water (20 mL) and extracted with ethyl acetate (3×20 mL). The combined organic layers were washed with brine (30 mL) and dried over anhydrous sodium sulfate, filtered and the filtrate was concentrated in vacuo to give the title compound (180 mg) as a yellow solid. LC-MS (ESI⁺) m/z 581.3 (M+H)⁺.

### (1—(Cyclopropylmethyl)-7-(2-ethyl-6-methylpyridin-3-yl)-2-(1,2,5,6-tetrahydropyridin-3-yl)-1H-indol-5-yl)(1-ethylpyrrolo[3,4-c]pyrazol-5(1H,4H,6H)-yl)methanone (Intermediate SM)

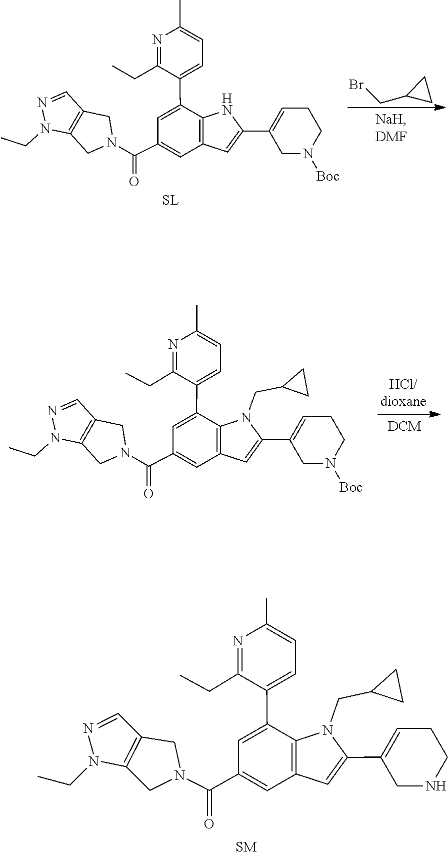

### Step 1—Tert-butyl 3-(1-(cyclopropylmethyl)-5-(1-ethyl-1,4,5,6-tetrahydropyrrolo[3,4-c]pyrazole-5-carbonyl)-7-(2-ethyl-6-methylpyridin-3-yl)-1H-indol-2-yl)-5,6-dihydropyridine-1(2H)-carboxylate

To a solution of tert-butyl 3-(5-(1-ethyl-1,4,5,6-tetrahydropyrrolo[3,4-c]pyrazole-5-carbonyl)-7-(2-ethyl-6-methylpyridin-3-yl)-1H-indol-2-yl)-5,6-dihydropyridine-1(2H)-carboxylate (180 mg, 309 μmol, Intermediate SL) in DMF (2 mL) was added NaH (18.6 mg, 464 μmol, 60% dispersion in mineral oil), and (bromomethyl)cyclopropane (62.7 mg, 464 μmol). The mixture was stirred at 0° C. for 2 hr under N₂. On completion, the crude residue was purified by reverse-phase HPLC (0.8 g/L ammonium bicarbonate) to give the title compound (180 mg, 92% yield) as a yellow solid. ¹H NMR (400 MHz, DMSO-d6) δ=7.87 (s, 1H), 7.75-7.63 (m, 1H), 7.30-7.17 (m, 2H), 7.08 (s, 1H), 6.68-6.62 (m, 1H), 6.02-5.95 (m, 1H), 4.85-4.74 (m, 2H), 4.66-4.53 (m, 2H), 4.26-4.16 (m, 1H), 4.14-4.06 (m, 1H), 4.06-3.92 (m, 2H), 3.57-3.48 (m, 2H), 2.56-2.53 (m, 7H), 2.45 (d, J=8.5 Hz, 2H), 2.26 (s, 2H), 1.41 (s, 9H), 1.35 (t, J=7.2 Hz, 2H), 1.26 (t, J=7.5 Hz, 2H), 1.10-1.00 (m, 3H), 0.57-0.39 (m, 1H), 0.19-0.02 (m, 2H), −0.12-−0.27 (m, 2H).

### Step 2—(1—(Cyclopropylmethyl)-7-(2-ethyl-6-methylpyridin-3-yl)-2-(1,2,5,6-tetrahydropyridin-3-yl)-1H-indol-5-yl)(1-ethylpyrrolo[3,4-c]pyrazol-5(1H,4H,6H)-yl)methanone

A mixture of tert-butyl 3-(1-(cyclopropylmethyl)-5-(1-ethyl-1,4,5,6-tetrahydropyrrolo[3,4-c]pyrazole-5-carbonyl)-7-(2-ethyl-6-methylpyridin-3-yl)-1H-indol-2-yl)-5,6-dihydropyridine-1(2H)-carboxylate (180 mg, 283 μmol) in HCl (2 mL) and DCM (2 mL) was degassed and purged with N₂ three times. Then the mixture was stirred at 25° C. for 2 hrs. On completion, the crude residue was concentrated in vacuo to give the title compound (160 mg) as a yellow solid. LC-MS (ESI⁺) m/z 535.4 (M+H)⁺.

### Methyl 4-(1-tert-butoxycarbonylpyrrol-2-yl)-6-chloro-7-fluoro-1H-indole-2-carboxylate (Intermediate SN)

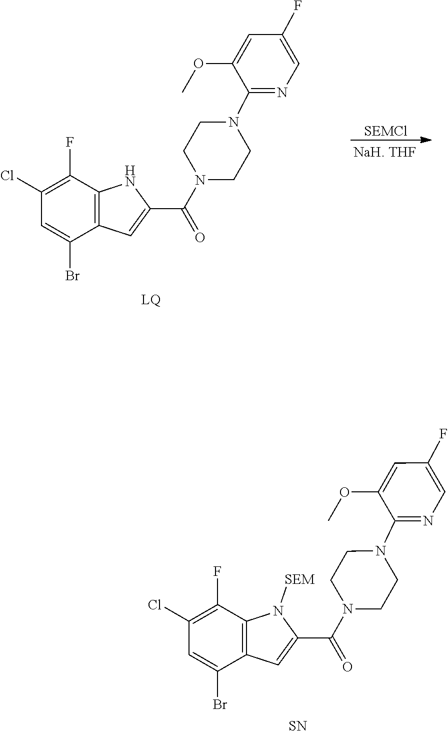 [SMILES: COc1cc(F)cnc1N1CCN(C(=O)c2cc3c(Br)cc(Cl)c(F)c3[nH]2)CC1 ; "SEMCl" ; COc1cc(F)cnc1N1CCN(C(=O)c2cc3c(Br)cc(Cl)c(F)c3n2COCC[Si](C)(C)C)CC1 ; "SN"]

To a solution of (4-bromo-6-chloro-7-fluoro-1H-indol-2-yl)-[4-(5-fluoro-3-methoxy-2-pyridyl)piperazin-1-yl]methanone (1 g, 2.06 mmol, Intermediate LQ) in THF (15 mL) was added NaH (123.52 mg, 3.09 mmol, 60% dispersion in mineral oil) at 0° C. The mixture was then stirred at 0° C. for 0.5 h. Then SEM-Cl (446.23 mg, 2.68 mmol, 473.70 μL) was added to the mixture at 0° C. Then the mixture was stirred at 20° C. for 1.5 h. Upon completion, the mixture was quenched with saturated NH₄Cl (20 mL), and then extracted with EA (15 mL×3). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 0˜19% Ethylacetate/Petroleum ether gradient @700 mL/min) to give the title compound (1.1 g, 87% yield) as a colorless oil. LC-MS (ESI⁺) m/z 615.0 (M+H)⁺.

### Tert-butyl 2-[6-chloro-7-fluoro-2-[4-(5-fluoro-3-methoxy-2-pyridyl)piperazine-1-carbonyl]-1-(2-trimethylsilylethoxymethyl)indol-4-yl]pyrrolidine-1-carboxylate (Intermediate SO)

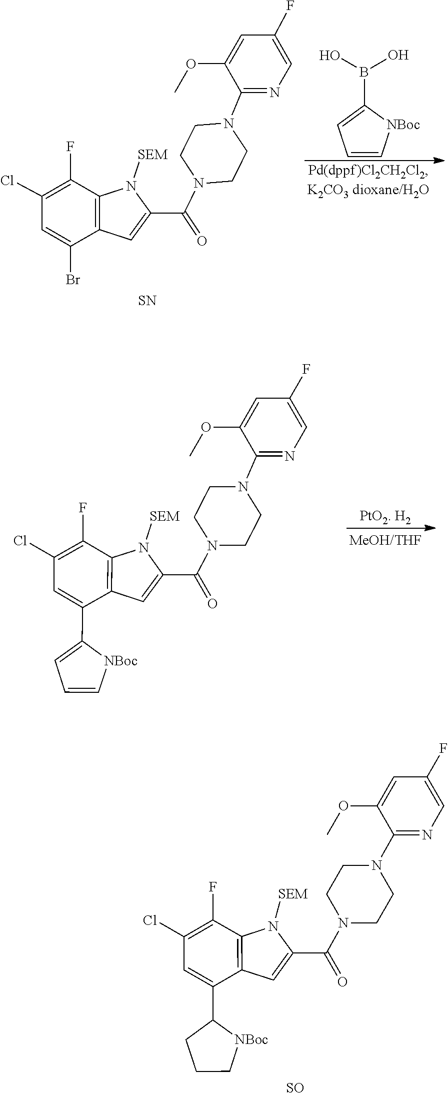

### Step 1—Tert-butyl 2-[6-chloro-7-fluoro-2-[4-(5-fluoro-3-methoxy-2-pyridyl)piperazine-1-carbonyl]-1-(2-trimethylsilylethoxymethyl)indol-4-yl]pyrrole-1-carboxylate

To a solution of [4-bromo-6-chloro-7-fluoro-1-(2-trimethylsilylethoxymethyl)indol-2-yl]-[4-(5-fluoro-3-methoxy-2-pyridyl)piperazin-1-yl]methanone (1 g, 2 mmol, Intermediate SN) and (1-tert-butoxycarbonylpyrrol-2-yl)boronic acid (343 mg, 1.62 mmol, CAS #135884-31-0) in dioxane (15 mL) and H₂O (1.5 mL) was added Pd(dppf)Cl₂ CH₂Cl₂ (133 mg, 162.35 μmol) and K₂CO₃ (673 mg, 4.87 mmol). The mixture was then stirred at 80° C. for 4 h. Upon completion, the aqueous phase was separated and the organic layer was filtered and concentrated under reduced pressure to give a residue. The residue was purified by flash silica gel chromatography (ISCO®; 20 g SepaFlash® Silica Flash Column, Eluent of 0˜19% EytOAc/Petroleum ether gradient @65 mL/min) to give the title compound (950 mg, 78% yield) as a white solid. LC-MS (ESI⁺) m/z 702.4 (M+H)⁺.

### Step 2—Tert-butyl 2-[6-chloro-7-fluoro-2-[4-(5-fluoro-3-methoxy-2-pyridyl)piperazine-1-carbonyl]-1-(2-trimethylsilylethoxymethyl)indol-4-yl]pyrrolidine-1-carboxylate

To a mixture of PtO₂ (150 mg, 661 μmol) in MeOH (10 mL) and was added tert-butyl 2-[6-chloro-7-fluoro-2-[4-(5-fluoro-3-methoxy-2-pyridyl)piperazine-1-carbonyl]-1-(2-trimethylsilylethoxymethyl)indol-4-yl]pyrrole-1-carboxylate (950 mg, 1.35 mmol) in THF (10 mL) under N₂. The suspension was degassed under vacuum and purged with H₂ several times. The mixture was stirred under H₂ (40 Psi) at 40° C. for 48 h. Upon completion, the mixture was filtered and the filtrate was concentrated under reduced pressure to give the residue. The residue was purified by flash silica gel chromatography (ISCO®; 12 g SepaFlash® Silica Flash Column, Eluent of 0˜32% Ethyl acetate/Petroleum ether gradient @60 mL/min) to give the title compound (650 mg, 64% yield) as a colorless gum. LC-MS (ESI⁺) m/z 706.3 (M+H)⁺; ¹H NMR (400 MHz, DMSO-d₆) δ=7.93 (d, J=2.0 Hz, 1H), 7.52 (dd, J=2.0, 10.4 Hz, 1H), 7.18-6.87 (m, 2H), 5.81 (s, 2H), 5.38-5.13 (m, 1H), 3.99 (s, 3H), 3.97-3.72 (m, 5H), 3.68-3.51 (m, 3H), 3.46 (s, 2H), 2.65 (s, 3H), 2.56-2.40 (m, 1H), 2.06-1.80 (m, 3H), 1.54 (s, 3H), 1.18-1.06 (m, 5H), 0.91 (t, J=7.6 Hz, 2H), 0.00 (s, 9H).

### 5-Bromo-4-methoxy-2-methylpyridine (CAS #886372-61-8) (Intermediate SP)

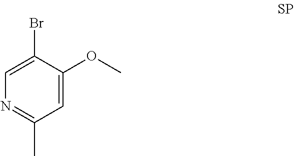

### 3-Bromopyridine-4-carbonitrile (CAS #13958-98-0) (Intermediate SQ)

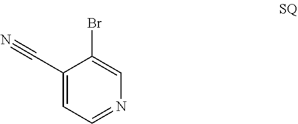

### 3-Bromopyridazine (CAS #88491-61-6) (Intermediate SR)

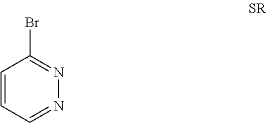

### 2-Bromo-1-methyl-1H-imidazole (CAS #16681-59-7) (Intermediate SS)

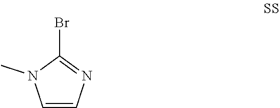

### Methyl 7-bromo-2-(1-(tert-butoxycarbonyl)-1,2,5,6-tetrahydropyridin-3-yl)-3-fluoro-1H-indole-5-carboxylate (Intermediate SV)

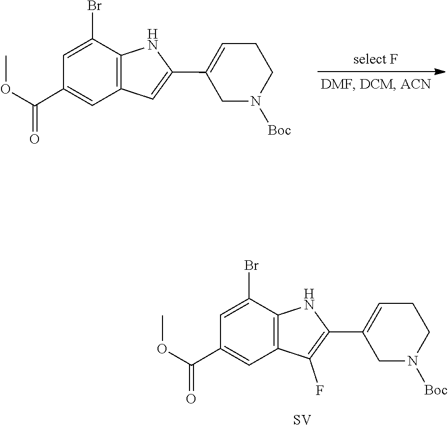 [SMILES: COC(=O)c1cc(Br)c2[nH]c(C3=CCCN(C(=O)OC(C)(C)C)C3)cc2c1 ; COC(=O)c1cc(Br)c2[nH]c(C3=CCCN(C(=O)OC(C)(C)C)C3)c(F)c2c1 ; "SV"]

To a solution of methyl 7-bromo-2-(1-(tert-butoxycarbonyl)-1,2,5,6-tetrahydropyridin-3-yl)-1H-indole-5-carboxylate (6 g, 14 mmol, synthesized via Steps 1-2 of Intermediate NM) in DCM (60 mL) and ACN (60 mL) was added dropwise 1-(chloromethyl)-4-fluoro-1,4-diazoniabicyclo[2.2.2]octane ditetrafluoroborate (2.44 g, 6.89 mmol, CAS #140681-55-6) in DMF (10 mL) at 0° C. and the mixture was stirred at 0° C. for 10 min. On completion, the reaction mixture was quenched with water (200 mL) and extracted with dichloromethane (3×100 mL). The combined organic layers were washed with brine (2×100 mL) and dried over anhydrous sodium sulfate, filtered and the filtrate was concentrated in vacuo to give the crude residue. The crude residue was purified by reversed-phase HPLC (0.8 g/L ammonium bicarbonate) to give the title compound (1.8 g, 24% yield) as a yellow solid. LC-MS (ESI⁺) m/z 453.0 (M+H)⁺; ¹H NMR (400 MHz, CDCl³-d) δ=8.27 (s, 1H), 8.06 (d, J=0.8 Hz, 1H), 7.97 (s, 1H), 6.32 (s, 1H), 4.43 (d, J=1.6 Hz, 2H), 3.94 (s, 3H), 3.60 (t, J=5.6 Hz, 2H), 2.40 (s, 2H), 1.51 (s, 9H).

### Methyl 2-(1-(tert-butoxycarbonyl)-1,2,5,6-tetrahydropyridin-3-yl)-3-fluoro-7-(4,4,5,5-tetramethyl-1,3,2-dioxaborolan-2-yl)-1H-indole-5-carboxylate (Intermediate SW)

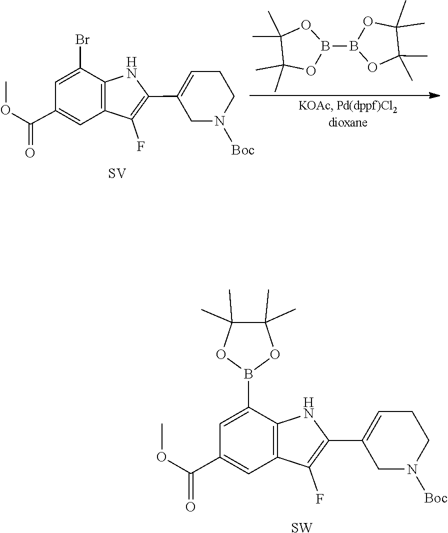

A mixture of methyl 7-bromo-2-(1-(tert-butoxycarbonyl)-1,2,5,6-tetrahydropyridin-3-yl)-3-fluoro-1H-indole-5-carboxylate (1.8 g, 4.0 mmol, Intermediate SV), 4,4,4′,4′,5,5,5′,5′-octamethyl-2,2′-bi(1,3,2-dioxaborolane) (3.03 g, 11.9 mmol), KOAc (1.17 g, 11.9 mmol), and Pd(dppf)Cl₂ (324 mg, 397 μmol) in dioxane (20 mL) was degassed and purged with N₂ three times. Then the mixture was stirred at 80° C. for 12 hrs under N₂ atmosphere. On completion, the reaction mixture was concentrated in vacuo to give the residue. The residue was purified by column chromatography (SiO₂, Petroleum ether/Ethyl acetate=1/0 to 3/1) to give the title compound (1.3 g, 56% yield) as a yellow solid. LC-MS (ESI⁺) m/z 501.1 (M+H)⁺.

### 2-(1-(Tert-butoxycarbonyl)-1,2,5,6-tetrahydropyridin-3-yl)-7-(2-ethyl-6-methylpyridin-3-yl)-3-fluoro-1H-indole-5-carboxylic acid (Intermediate SX)

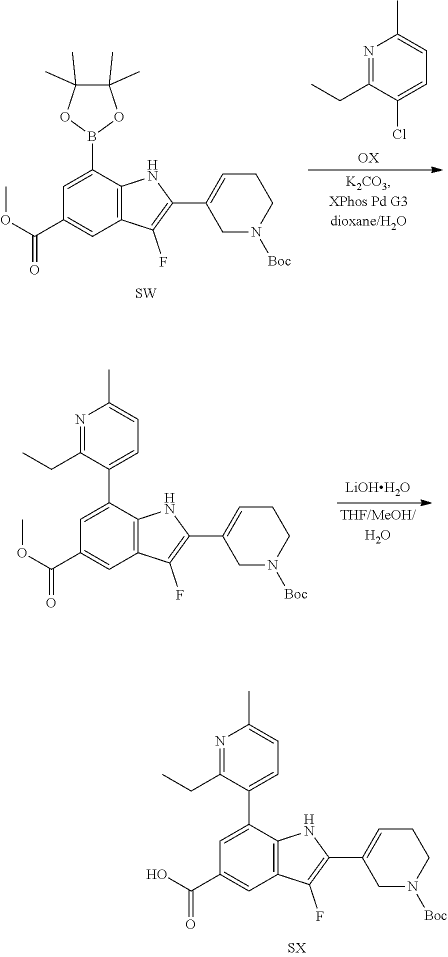

### Step 1—Methyl 2-(1-(tert-butoxycarbonyl)-1,2,5,6-tetrahydropyridin-3-yl)-7-(2-ethyl-6-methylpyridin-3-yl)-3-fluoro-1H-indole-5-carboxylate

A mixture of methyl 2-(1-(tert-butoxycarbonyl)-1,2,5,6-tetrahydropyridin-3-yl)-3-fluoro-7-(4,4,5,5-tetramethyl-1,3,2-dioxaborolan-2-yl)-1H-indole-5-carboxylate (800 mg, 1.60 mmol, Intermediate SW), 3-chloro-2-ethyl-6-methylpyridine (249 mg, 1.60 mmol, Intermediate OX), XPhos Pd G3 (135 mg, 159 μmol), and K₂CO₃ (663 mg, 4.80 mmol) in dioxane (10 mL) and H₂O (2 mL) was degassed and purged with N₂ three times. Then the mixture was stirred at 80° C. for 2 hrs under N₂ atmosphere. On completion, the reaction mixture was concentrated in vacuo to give the residue. The residue was purified by column chromatography (SiO₂, Petroleum ether/Ethyl acetate=1/0 to 1/1) to give the title compound (600 mg, 64% yield) as a yellow solid. LC-MS (ESI⁺) m/z 494.3 (M+H)⁺.

### Step 2—2-(1-(Tert-butoxycarbonyl)-1,2,5,6-tetrahydropyridin-3-yl)-7-(2-ethyl-6-methylpyridin-3-yl)-3-fluoro-1H-indole-5-carboxylic acid

To a solution of methyl 2-(1-(tert-butoxycarbonyl)-1,2,5,6-tetrahydropyridin-3-yl)-7-(2-ethyl-6-methylpyridin-3-yl)-3-fluoro-1H-indole-5-carboxylate (600 mg, 1.22 mmol) in THF (8 mL), MeOH (2 mL) and H₂O (2 mL) was added LiOH·H₂O (510 mg, 12.2 mmol). The mixture was then stirred at 40° C. for 12 hrs. On completion, the reaction mixture was quenched with HCl (1M) until pH=2-3, and extracted with ethyl acetate (3×30 mL). The combined organic layers were washed with brine (2×20 mL) and dried over anhydrous sodium sulfate, filtered and the filtrate was concentrated in vacuo to give the title compound (550 mg) as a yellow solid. LC-MS (ESI⁺) m/z 480.1 (M+H)⁺; ¹H NMR (400 MHz, DMSO-d₆) δ=12.93-12.43 (m, 1H), 11.08-10.72 (m, 1H), 8.45-7.87 (m, 2H), 7.78-7.40 (m, 2H), 6.55-6.37 (m, 1H), 4.33 (s, 2H), 3.46 (s, 4H), 2.91-2.70 (m, 3H), 2.25 (s, 2H), 1.42 (s, 9H), 1.07-1.00 (m, 3H).

### Tert-butyl 3-(5-(4-cyclopropylpiperazine-1-carbonyl)-7-(2-ethyl-6-methylpyridin-3-yl)-3-fluoro-1H-indol-2-yl)-5,6-dihydropyridine-1(2H)-carboxylate (Intermediate SY)

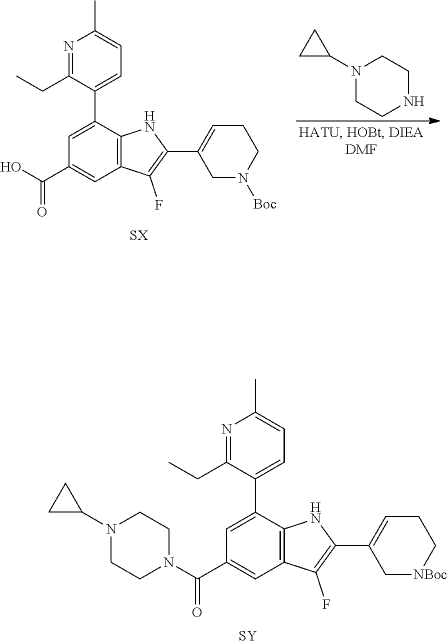

To a solution of 2-(1-(tert-butoxycarbonyl)-1,2,5,6-tetrahydropyridin-3-yl)-7-(2-ethyl-6-methylpyridin-3-yl)-3-fluoro-1H-indole-5-carboxylic acid (300 mg, 625 μmol, Intermediate SX) in DMF (3 mL) was added HATU (285 mg, 750 μmol), HOBt (127 mg, 938 μmol) and DIEA (323 mg, 2.50 mmol). After addition, the mixture was stirred at 25° C. for 10 min, and then 1-cyclopropylpiperazine (78.9 mg, 625 μmol, CAS #20327-23-5) was added and the mixture was stirred at 25° C. for 20 min. On completion, the reaction mixture was diluted with water (20 mL) and extracted with ethyl acetate (3×10 mL). The combined organic layers were washed with brine (2×10 mL) and dried over anhydrous sodium sulfate, filtered and the filtrate was concentrated in vacuo to give the title compound (350 mg) as a brown solid. LC-MS (ESI⁺) m/z 588.4 (M+H)⁺.

### Tert-butyl (1-(cyclopropylmethyl)-7-(2-ethyl-6-methylpyridin-3-yl)-3-fluoro-2-(1,2,5,6-tetrahydropyridin-3-yl)-1H-indol-5-yl)(4-cyclopropylpiperazin-1-yl)methanone (Intermediate SZ)

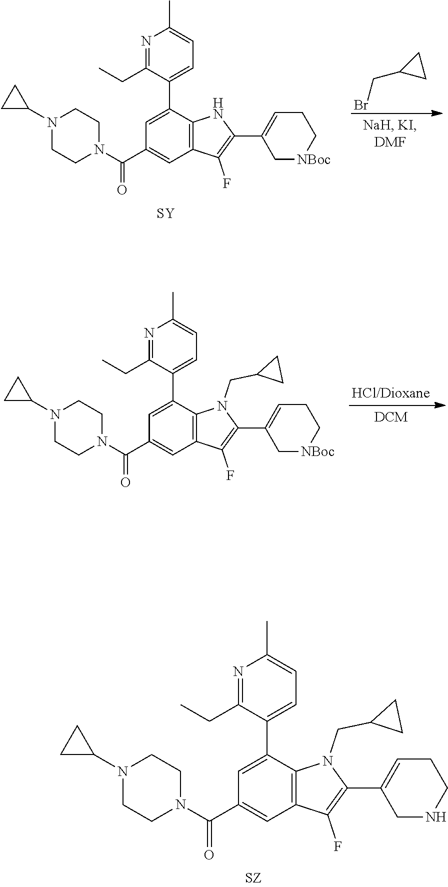

### Step 1—Tert-butyl 3-(1-(cyclopropylmethyl)-5-(4-cyclopropylpiperazine-1-carbonyl)-7-(2-ethyl-6-methylpyridin-3-yl)-3-fluoro-1H-indol-2-yl)-5,6-dihydropyridine-1(2H)-carboxylate

To a solution of tert-butyl 3-(5-(4-cyclopropylpiperazine-1-carbonyl)-7-(2-ethyl-6-methylpyridin-3-yl)-3-fluoro-1H-indol-2-yl)-5,6-dihydropyridine-1(2H)-carboxylate (300 mg, 510 μmol, Intermediate SY) in DMF (3 mL) was added NaH (40.8 mg, 1.02 mmol) at 0° C. After addition, the mixture was stirred at this temperature for 1 hr, and then (bromomethyl)cyclopropane (103 mg, 766 μmol, CAS #7051-34-5) and KI (8.47 mg, 51.0 μmol) was added at 0° C. The resulting mixture was stirred at 25° C. for 2 hrs. On completion, the reaction mixture was quenched with NH₄Cl (10 mL) and extracted with ethyl acetate (3×10 mL). The combined organic layers were washed with brine (2×5 mL) and dried over anhydrous sodium sulfate, filtered and the filtrate was concentrated in vacuo to give the title compound (350 mg) as a brown solid. LC-MS (ESI⁺) m/z 642.4 (M+H)⁺.

### Step 2—Tert-butyl (1-(cyclopropylmethyl)-7-(2-ethyl-6-methylpyridin-3-yl)-3-fluoro-2-(1,2,5,6-tetrahydropyridin-3-yl)-1H-indol-5-yl)(4-cyclopropylpiperazin-1-yl)methanone

To a solution of tert-butyl 3-(1-(cyclopropylmethyl)-5-(4-cyclopropylpiperazine-1-carbonyl)-7-(2-ethyl-6-methylpyridin-3-yl)-3-fluoro-1H-indol-2-yl)-5,6-dihydropyridine-1(2H)-carboxylate (300 mg, 467 μmol) in DCM (2 mL) was added HCl/dioxane (2 M, 2 mL). The mixture was then stirred at 25° C. for 1 hr. On completion, the reaction mixture was concentrated in vacuo to give the title compound (250 mg) as a brown solid. LC-MS (ESI⁺) m/z 542.4 (M+H)⁺.

### Tert-butyl 3-(5-(1-ethyl-1,4,5,6-tetrahydropyrrolo[3,4-c]pyrazole-5-carbonyl)-7-(1-ethyl-1H-pyrazol-5-yl)-1H-indol-2-yl)-5,6-dihydropyridine-1(2H)-carboxylate (Intermediate TA)

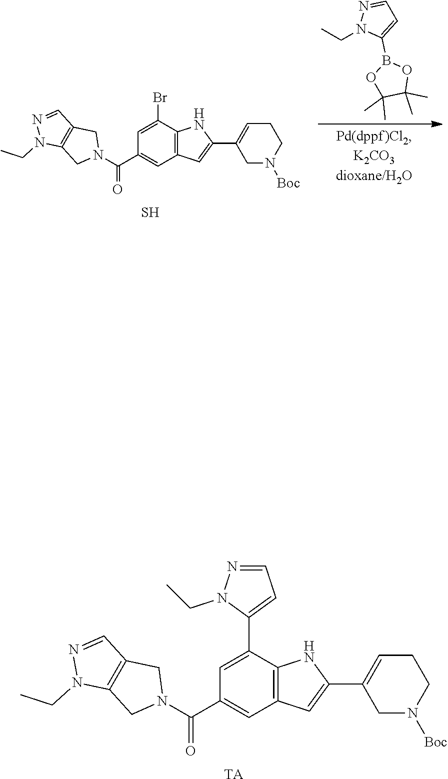

To a solution of tert-butyl 3-(7-bromo-5-(1-ethyl-1,4,5,6-tetrahydropyrrolo[3,4-c]pyrazole-5-carbonyl)-1H-indol-2-yl)-5,6-dihydropyridine-1(2H)-carboxylate (600.00 mg, 777.13 μmol, Intermediate SH) and 1-ethyl-5-(4,4,5,5-tetramethyl-1,3,2-dioxaborolan-2-yl)pyrazole (172.59 mg, 777.13 μmol, Intermediate QM) in dioxane (5 mL) and H₂O (1 mL) were added Pd(dppf)Cl₂ (56.86 mg, 77.71 μmol) and K₂CO₃ (322.21 mg, 2.33 mmol). The mixture was then stirred at 80° C. for 1 h. Upon completion, the mixture was diluted with H₂O (5 mL) and extracted with EtOAc (5 mL×2). The combined organic layers were dried over Na₂SO₄, filtered and concentrated under reduced pressure to give a residue. The residue was purified by flash silica gel chromatography (ISCO®; 4 g SepaFlash® Silica Flash Column, Eluent of 0˜100% EtOAc/Petroleum ether gradient @100 mL/min) to give the title compound (279.00 mg, 61% yield) as a yellow solid. LC-MS (ESI+) m/z 556.3 (M+H)⁺; ¹H NMR (400 MHz, DMSO-d₆) δ=11.02 (s, 1H), 7.89 (s, 1H), 7.60 (s, 1H), 7.41-7.07 (m, 2H), 6.70 (s, 1H), 6.60 (s, 1H), 6.42 (s, 1H), 4.78 (s, 2H), 4.58 (d, J=6.4 Hz, 2H), 4.25 (s, 2H), 4.13-3.92 (m, 4H), 3.48 (t, J=5.6 Hz, 2H), 2.26 (s, 2H), 1.44 (s, 9H), 1.38-1.25 (m, 3H), 1.24-1.15 (m, 3H).

### (1—(Cyclopropylmethyl)-7-(1-ethyl-1H-pyrazol-5-yl)-2-(1,2,5,6-tetrahydropyridin-3-yl)-1H-indol-5-yl)(1-ethylpyrrolo[3,4-c]pyrazol-5(1H,4H,6H)-yl)methanone (Intermediate TB)

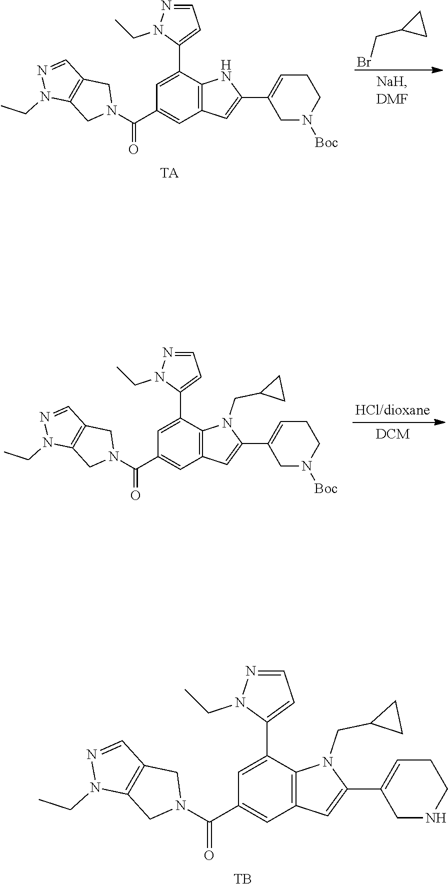

### Step 1—Tert-butyl 3-(1-(cyclopropylmethyl)-5-(1-ethyl-1,4,5,6-tetrahydropyrrolo[3,4-c]pyrazole-5-carbonyl)-7-(1-ethyl-1H-pyrazol-5-yl)-1H-indol-2-yl)-5,6-dihydropyridine-1(2H)-carboxylate

To a solution of tert-butyl 3-(5-(1-ethyl-1,4,5,6-tetrahydropyrrolo[3,4-c]pyrazole-5-carbonyl)-7-(1-ethyl-1H-pyrazol-5-yl)-1H-indol-2-yl)-5,6-dihydropyridine-1(2H)-carboxylate (279.00 mg, 502.10 μmol, Intermediate TA) in DMF (5 mL) was added dropwise NaH (24.10 mg, 602.5 μmol, 60% dispersion in mineral oil) at 20° C. for 1 h. After addition bromomethylcyclopropane (74.56 mg, 552.3 μmol, 52.73 μL, CAS #7051-34-5) was added dropwise at 20° C. The resulting mixture was stirred at 20° C. for 11 h. The reaction mixture was quenched with NH₄Cl (3 mL) at 0° C., and then diluted with H₂O (3 mL) and extracted with EtOAc (10 mL×2). The combined organic layers were dried over Na₂SO₄, filtered and concentrated under reduced pressure to give a residue. The residue was purified by flash silica gel chromatography (ISCO®; 4 g SepaFlash® Silica Flash Column, Eluent of 0-100% EtOAc/Petroleum ether gradient @100 mL/min) to give the title compound (250.00 mg, 57% yield) as a yellow solid. LC-MS (ESI⁺) m/z 610.8 (M+H)⁺.

### Step 2—(1—(Cyclopropylmethyl)-7-(1-ethyl-1H-pyrazol-5-yl)-2-(1,2,5,6-tetrahydropyridin-3-yl)-1H-indol-5-yl)(1-ethylpyrrolo[3,4-c]pyrazol-5(1H,4H,6H)-yl)methanone

To a solution of tert-butyl 3-(1-(cyclopropylmethyl)-5-(1-ethyl-1,4,5,6-tetrahydropyrrolo[3,4-c]pyrazole-5-carbonyl)-7-(1-ethyl-1H-pyrazol-5-yl)-1H-indol-2-yl)-5,6-dihydropyridine-1(2H)-carboxylate (250.00 mg, 410.00 μmol) in DCM (2 mL) was added HCl/dioxane (2 M, 2 mL). The mixture was then stirred at 25° C. for 1 h. Upon completion, the mixture was concentrated under reduced pressure to give a residue. The residue was extracted with EtOAc (5 mL×2) and washed with sodium hydrogen carbonate (5 mL). The combined organic layers were dried over Na₂SO₄, filtered and concentrated under reduced pressure to give the title compound (200.00 mg, 88% yield) as a yellow gum. LC-MS (ESI⁺) m/z 510.3 (M+H)⁺; ¹H NMR (400 MHz, DMSO-d₆) δ=7.92 (s, 1H), 7.62 (s, 1H), 7.36-7.10 (m, 2H), 6.61 (s, 1H), 6.47 (d, J=1.6 Hz, 1H), 5.96 (s, 1H), 4.78 (d, J=4.0 Hz, 2H), 4.58 (s, 2H), 4.11 (d, J=7.2 Hz, 1H), 3.99 (d, J=7.2 Hz, 1H), 3.95-3.84 (m, 1H), 3.78 (d, J=7.6 Hz, 2H), 3.57 (s, 2H), 2.84 (s, 2H), 2.15 (s, 2H), 1.35 (t, J=7.2 Hz, 2H), 1.31-1.17 (m, 6H), 0.66-0.50 (m, 1H), 0.15 (d, J=1.2 Hz, 2H), 0.00-−0.21 (m, 2H).

### Acetyl chloride (CAS #75-36-5) (Intermediate TC)

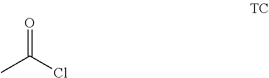

### Ethyl 2-chloro-2-oxo-acetate (CAS #4755-77-5) (Intermediate TD)

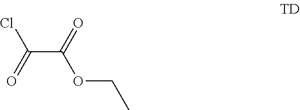

### Tert-butyl 5-[7-(3-ethyl-4-pyridyl)-5-[4-(5-fluoro-3-methoxy-2-pyridyl)piperazine-1-carbonyl]-1H-indol-2-yl]-3,6-dihydro-2H-pyridine-1-carboxylate (Intermediate TE)

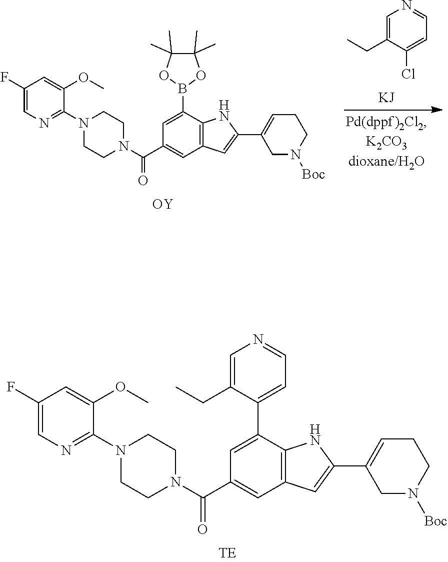

A mixture of tert-butyl 5-[5-[4-(5-fluoro-3-methoxy-2-pyridyl)piperazine-1-carbonyl]-7-(4,4,5,5-tetramethyl-1,3,2-dioxaborolan-2-yl)-1H-indol-2-yl]-3,6-dihydro-2H-pyridine-1-carboxylate (300 mg, 453.47 μmol, Intermediate OY), 4-chloro-3-ethyl-pyridine (96 mg, 680.20 μmol, Intermediate KJ), XPhos Pd G3 (38 mg, 45.35 μmol), K₂CO₃ (188 mg, 1.36 mmol) in dioxane (5 mL) and H₂O (1 mL) was degassed and purged with N₂ three times. Then the mixture was stirred at 100° C. for 1 h under N₂ atmosphere. Upon completion, the aqueous phase was removed and the organic phase was filtered and the filtrate was concentrated under reduced pressure to give the residue. The residue was purified by flash silica gel chromatography (ISCO®; 4 g SepaFlash® Silica Flash Column, Eluent of 0˜85% EtOAc/Petroleum ether gradient @40 mL/min) to give the title compound (290 mg, 77% yield) as light yellow oil. LC-MS (ESI⁺) m/z 641.4 (M+H)⁺; ¹H NMR (400 MHz, DMSO-d₆) δ=10.92-10.72 (m, 1H), 8.60 (s, 1H), 8.49 (d, J=5.2 Hz, 1H), 7.78 (d, J=2.4 Hz, 1H), 7.67 (s, 1H), 7.39-7.31 (m, 1H), 7.27 (d, J=4.8 Hz, 1H), 6.95 (s, 1H), 6.64-6.55 (m, 2H), 4.29-4.19 (m, 2H), 3.83 (s, 3H), 3.71-3.55 (m, 4H), 3.51-3.43 (m, 4H), 3.26-3.21 (m, 4H), 2.27-2.16 (m, 2H), 1.43 (s, 9H), 0.95 (t, J=7.6 Hz, 3H).

### [1—(Cyclopropylmethyl)-7-(3-ethyl-4-pyridyl)-2-(1,2,3,6-tetrahydropyridin-5-yl)indol-5-yl]-[4-(5-fluoro-3-methoxy-2-pyridyl)piperazin-1-yl]methanone (Intermediate TF)

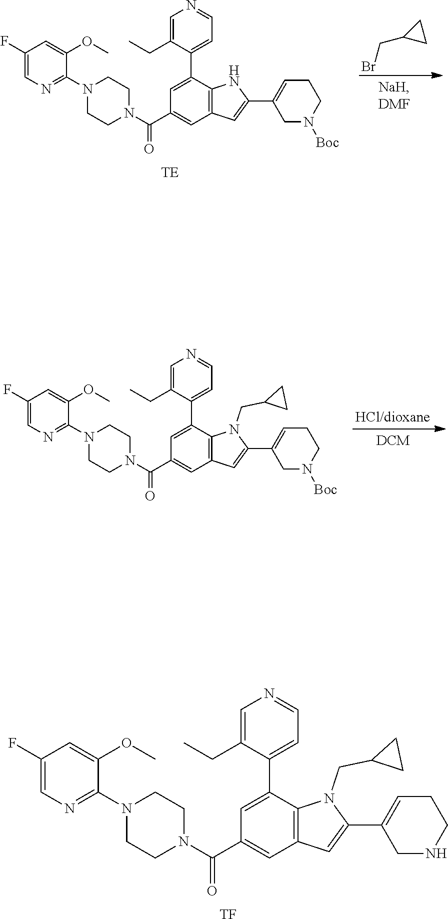

### Step 1—Tert-butyl 5-[1-(cyclopropylmethyl)-7-(3-ethyl-4-pyridyl)-5-[4-(5-fluoro-3-methoxy-2-pyridyl)piperazine-1-carbonyl]indol-2-yl]-3,6-dihydro-2H-pyridine-1-carboxylate

To a solution of tert-butyl 5-[7-(3-ethyl-4-pyridyl)-5-[4-(5-fluoro-3-methoxy-2-pyridyl)piperazine-1-carbonyl]-1H-indol-2-yl]-3,6-dihydro-2H-pyridine-1-carboxylate (290 mg, 453 gmol, Intermediate TE) in DMF (5 mL) was added NaH (22 mg, 543.12 μmol, 60% dispersion in mineral oil) and the mixture was stirred at 20° C. for 0.5 h. Then bromomethylcyclopropane (91.65 mg, 678.9 μmol, 64.82 μL) was added and the mixture was stirred at 20° C. for 12 h. Upon completion, the mixture was quenched with saturated NH₄Cl (10 mL) at 0° C., and then extracted with EtOAc (10 mL×3). The combined organic layers were dried over Na₂SO₄, filtered and concentrated under reduced pressure to give a residue. The residue was purified by flash silica gel chromatography (ISCO®; 4 g SepaFlash® Silica Flash Column, Eluent of 0˜75% EtOAc/Petroleum ether gradient @45 mL/min) to give the title compound (270 mg, 86% yield) as yellow oil. LC-MS (ESI⁺) m/z 695.4 (M+H)⁺.

### Step 2—[1—(Cyclopropylmethyl)-7-(3-ethyl-4-pyridyl)-2-(1,2,3,6-tetrahydropyridin-5-yl)indol-5-yl]-[4-(5-fluoro-3-methoxy-2-pyridyl)piperazin-1-yl]methanone

To a solution of tert-butyl 5-[1-(cyclopropylmethyl)-7-(3-ethyl-4-pyridyl)-5-[4-(5-fluoro-3-methoxy-2-pyridyl)piperazine-1-carbonyl]indol-2-yl]-3,6-dihydro-2H-pyridine-1-carboxylate (262 mg, 377 μmol) in DCM (3 mL) was added HCl/dioxane (2 M, 3 mL) and the mixture was stirred at 20° C. for 12 h. Upon completion, the pH value was adjusted to 8 by adding saturated NaHCO₃ solution. Then the mixture was extracted with DCM (5 mL×3). The combined organic layers were dried over Na₂SO₄, filtered and concentrated under reduced pressure to give the title compound (195 mg, 87% yield) as gray oil. LC-MS (ESI⁺) m/z 595.4 (M+H)⁺.

### Tert-butyl 3-(5-(1-ethyl-1,4,5,6-tetrahydropyrrolo[3,4-c]pyrazole-5-carbonyl)-7-(6-ethyl-2-methylpyridin-3-yl)-1H-indol-2-yl)-5,6-dihydropyridine-1(2H)-carboxylate (Intermediate TG)

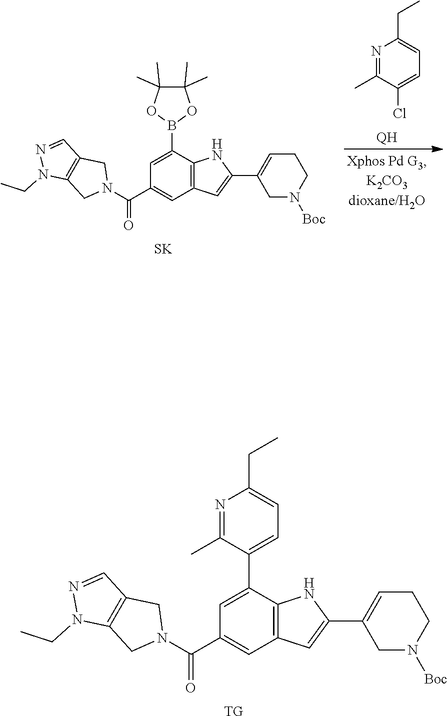

A mixture of tert-butyl 3-(5-(1-ethyl-1,4,5,6-tetrahydropyrrolo[3,4-c]pyrazole-5-carbonyl)-7-(4,4,5,5-tetramethyl-1,3,2-dioxaborolan-2-yl)-1H-indol-2-yl)-5,6-dihydropyridine-1(2H)-carboxylate (2.30 g, 3.91 mmol, Intermediate SK), 3-chloro-6-ethyl 2-methyl-pyridine (609.24 mg, 3.91 mmol, Intermediate QH), XPhos Pd G₃ (331.37 mg, 391.48 μmol), K₂CO₃ (1.62 g, 11.74 mmol) and H₂O (5 mL) in dioxane (15 mL) was degassed and purged with N₂ three times. Then the mixture was stirred at 80° C. for 2 h under N₂. Upon completion, the reaction was filtered and the filtrate was concentrated by vacuum distillation to obtain the crude product. The residue was purified by column chromatography (SiO₂, Petroleum ether/EtOAc=100/1 to 1/1) to give the title compound (2.00 g, 85% yield) as a yellow solid. LC-MS (ESI⁺) m/z 581.2 (M+H)⁺.

### (1—(Cyclopropylmethyl)-7-(6-ethyl-2-methylpyridin-3-yl)-2-(1,2,5,6-tetrahydropyridin-3-yl)-1H-indol-5-yl)(1-ethylpyrrolo[3,4-c]pyrazol-5(1H,4H,6H)-yl)methanone (Intermediate TH)

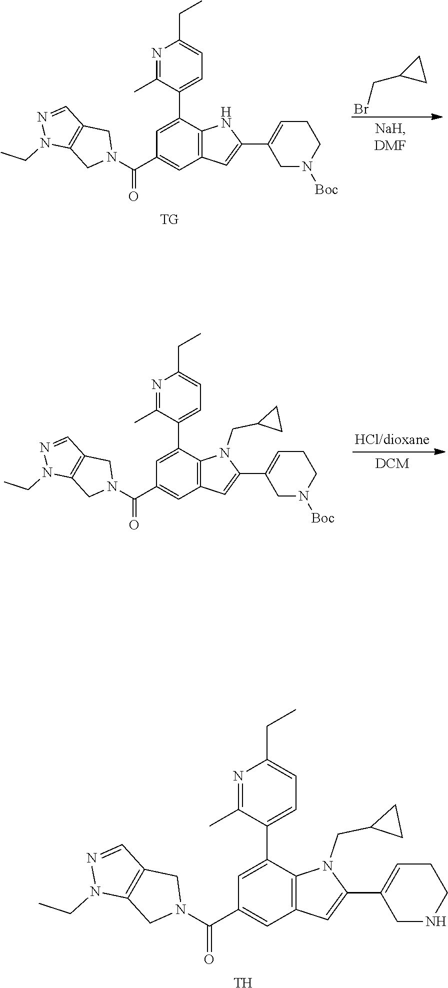 [SMILES: CCc1ccc(-c2cc(C(=O)N3Cc4cnn(CC)c4C3)cc3cc(C4=CCCN(C(=O)OC(C)(C)C)C4)[nH]c23)c(C)n1 ; BrCC1CC1 ; "TG" ; CCc1ccc(-c2cc(C(=O)N3Cc4cnn(CC)c4C3)cc3cc(C4=CCCN(C(=O)OC(C)(C)C)C4)n(CC4CC4)c23)c(C)n1 ; CCc1ccc(-c2cc(C(=O)N3Cc4cnn(CC)c4C3)cc3cc(C4=CCCNC4)n(CC4CC4)c23)c(C)n1 ; "TH"]

### Step 1—Tert-butyl 3-(1-(cyclopropylmethyl)-5-(1-ethyl-1,4,5,6-tetrahydropyrrolo[3,4-c]pyrazole-5-carbonyl)-7-(6-ethyl-2-methylpyridin-3-yl)-1H-indol-2-yl)-5,6-dihydropyridine-1(2H)-carboxylate

To a solution of tert-butyl 3-(5-(1-ethyl-1,4,5,6-tetrahydropyrrolo[3,4-c]pyrazole-5-carbonyl)-7-(6-ethyl-2-methylpyridin-3-yl)-1H-indol-2-yl)-5,6-dihydropyridine-1(2H)-carboxylate (510.00 mg, 878.22 μmol, Intermediate TG) in DMF (5 mL) was added dropwise NaH (52.69 mg, 1.32 mmol, 60% dispersion mineral oil) at 20° C. for 1 h. Then, bromomethylcyclopropane (177.84 mg, 1.32 mmol, 125.77 μL, CAS #7051-34-5) was added dropwise at 20° C. and the mixture was stirred at 20° C. for 11 h. Upon completion, the mixture was quenched with NH₄Cl (3 mL) at 0° C., and then diluted with H₂O (3 mL) and extracted with EtOAc (10 mL×2). The combined organic layers were dried over Na₂SO₄, filtered and concentrated under reduced pressure to give the title compound (530.00 mg) was obtained as a yellow gum. LC-MS (ESI⁺) m/z 635.4 (M+H)⁺.

### Step 2—(1—(Cyclopropylmethyl)-7-(6-ethyl-2-methylpyridin-3-yl)-2-(1,2,5,6-tetrahydropyridin-3-yl)-1H-indol-5-yl)(1-ethylpyrrolo[3,4-c]pyrazol-5(1H,4H,6H)-yl)methanone

To a solution of tert-butyl 3-(1-(cyclopropylmethyl)-5-(1-ethyl-1,4,5,6-tetrahydropyrrolo[3,4-c]pyrazole-5-carbonyl)-7-(6-ethyl-2-methylpyridin-3-yl)-1H-indol-2-yl)-5,6-dihydropyridine-1(2H)-carboxylate (530.00 mg, 834.90 μmol) in DCM (3 mL) was added HCl/dioxane (2 M, 3.18 mL). The mixture was then stirred at 20° C. for 1 h. Upon completion, the mixture was concentrated under reduced pressure to give a residue. The residue was extracted with EtOAc (5 mL×2) and washed with sodium hydrogen carbonate (5 mL). The combined organic layers were dried over Na₂SO₄, filtered and concentrated under reduced pressure to give the title compound (446.00 mg) as a yellow solid. LC-MS (ESI⁺) m/z 535.4 (M+H)⁺; ¹H NMR (400 MHz, DMSO-d₆) δ=8.02-7.78 (m, 1H), 7.76-7.64 (m, 1H), 7.35-7.14 (m, 2H), 7.13-6.97 (m, 1H), 6.72-6.40 (m, 1H), 5.95 (s, 1H), 4.78 (d, J=9.2 Hz, 2H), 4.58 (s, 2H), 4.13-3.97 (m, 2H), 3.68-3.47 (m, 2H), 2.94-2.79 (m, 4H), 2.79-2.69 (m, 2H), 2.21 (d, J=6.4 Hz, 3H), 2.15 (s, 2H), 2.08-2.07 (m, 1H), 1.39-1.24 (m, 6H), 0.52-0.43 (m, 1H), 0.18-0.00 (m, 2H), −0.21 (s, 2H).

### Cyclohexen-1-ylboronic acid (CAS #89490-05-1) (Intermediate TI)

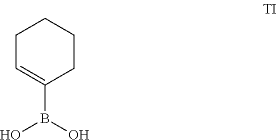

### (7-Bromo-2-(1,2,5,6-tetrahydropyridin-3-yl)-1H-indol-5-yl)(1-ethyl-4,6-dihydropyrrolo[3,4-c]pyrazol-5(1H)-yl)methanone (Intermediate TJ)

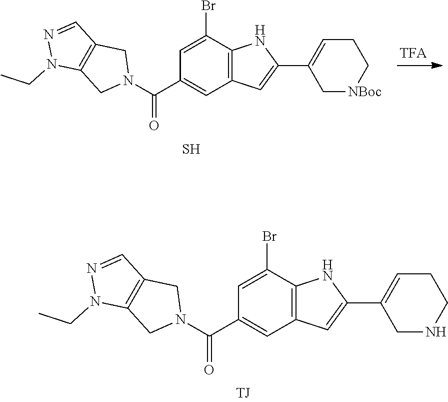

To a solution of tert-butyl 5-[7-bromo-5-(1-ethyl-4,6-dihydropyrrolo[3,4-c]pyrazole-5-carbonyl)-1H-indol-2-yl]-3,6-dihydro-2H-pyridine-1-carboxylate (1 g, 1.85 mmol, Intermediate SH) in TFA (10 mL) and the mixture was stirred at 25° C. for 1 hr. Upon completion, the reaction mixture was filtered and concentrated under reduced pressure to give the title compound (900 mg, 71% yield, TF) as a deep yellow oil. LC-MS (ESI⁺) m/z 280.0 (M+H)⁺.

### 1-(3-(7-Bromo-5-(1-ethyl-1,4,5,6-tetrahydropyrrolo[3,4-c]pyrazole-5-carbonyl)-1H-indol-2-yl)-5,6-dihydropyridin-1(2H)-yl)-3-(1H-1,2,3-triazol-1-yl)propan-1-one (Intermediate TK)

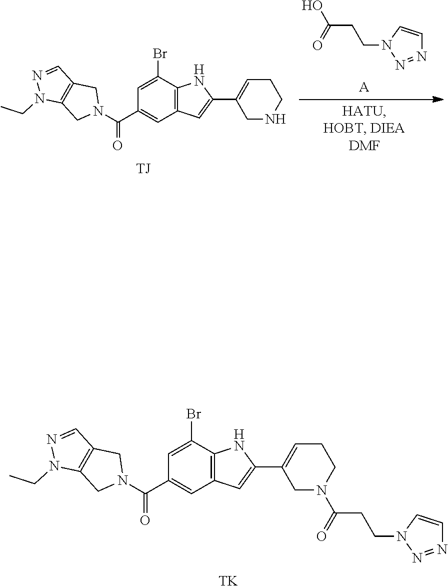

To a solution of [7-bromo-2-(1,2,3,6-tetrahydropyridin-5-yl)-1H-indol-5-yl]-(1-ethyl-4,6-dihydropyrrolo[3,4-c]pyrazol-5-yl)methanone (900 mg, 2.04 mmol, Intermediate TJ) and 3-(triazol-1-yl)propanoic acid (288 mg, 2.04 mmol, Intermediate A) in DMF (10 mL) was added HATU (932 mg, 2.45 mmol) and HOBt (552 mg, 4.09 mmol) and DIEA (1.06 g, 8.18 mmol). The mixture was then stirred at 25° C. for 10 mins. Upon completion, the mixture was quenched with H₂O (10 mL) and extracted with EA (20 mL×3). The combined organic layers were washed with brine (20 mL×3), dried over Na₂SO₄, filtered and concentrated under reduced pressure to give the title compound (890 mg, 64% yield) as a yellow solid. LC-MS (ESI⁺) m/z 563.2 (M+H)⁺; ¹H NMR (400 MHz, DMSO-d6) δ=11.46-11.04 (m, 1H), 8.30-8.06 (m, 1H), 7.99-7.95 (m, 3H), 7.85-7.72 (m, 1H), 7.58-7.44 (m, 1H), 7.42 (s, 1H), 6.87-6.62 (m, 1H), 4.79-4.55 (m, 3H), 4.39 (d, J=8.8 Hz, 1H), 4.15-3.99 (m, 2H), 3.70-3.59 (m, 2H), 3.20-3.14 (m, 4H), 2.90 (s, 4H), 1.27-1.25 (m, 3H).

### 1-(3-(7-Bromo-1-(cyclopropylmethyl)-5-(1-ethyl-1,4,5,6-tetrahydropyrrolo[3,4-c]pyrazole-5-carbonyl)-1H-indol-2-yl)-5,6-dihydropyridin-1(2H)-yl)-3-(1H-1,2,3-triazol-1-yl)propan-1-one (Intermediate TL)

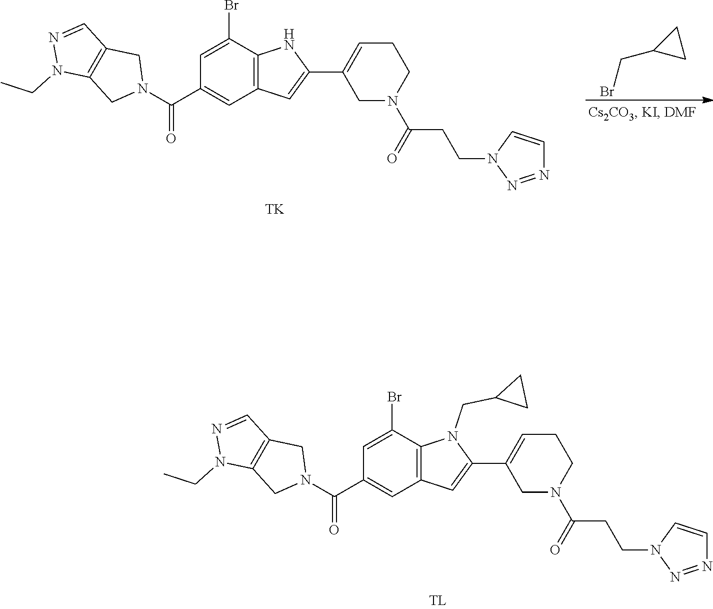 [SMILES: CCn1ncc2c1CN(C(=O)c1cc(Br)c3[nH]c(C4=CCCN(C(=O)CCn5ccnn5)C4)cc3c1)C2 ; BrCC1CC1 ; "TK" ; CCn1ncc2c1CN(C(=O)c1cc(Br)c3c(c1)cc(C1=CCCN(C(=O)CCn4ccnn4)C1)n3CC1CC1)C2]

To a solution of 1-[5-[7-bromo-5-(1-ethyl-4,6-dihydropyrrolo[3,4-c]pyrazole-5-carbonyl)-1H-indol-2-yl]-3,6-dihydro-2H-pyridin-1-yl]-3-(triazol-1-yl)propan-1-one (890 mg, 1.58 mmol, Intermediate TK) and bromomethylcyclopropane (319 mg, 2.37 mmol) in DMF (10 mL) was added Cs₂CO₃ (1.54 g, 4.74 mmol) and KI (26.2 mg, 157 μmol). The mixture was then stirred at 25° C. for 12 hrs. Upon completion, the mixture was quenched with MeOH (5 mL) and extracted with EA (20 mL×2). The combined organic layers were washed with brine (20 mL×2), dried over Na₂SO₄, filtered and concentrated under reduced pressure to give a residue. The residue was purified by column chromatography (SiO₂, DCM: MeOH=10:1) to give the title compound (970 mg, 62% yield) as a white solid. LC-MS (ESI⁺) m/z 617.2 (M+H)⁺.

### Cyclopropylboronic Acid (CAS #411235-57-9) (Intermediate TM)

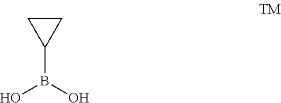

### (7-(6-Ethyl-2-methylpyridin-3-yl)-1-isobutyl-2-(1,2,5,6-tetrahydropyridin-3-yl)-1H-indol-5-yl)(1-ethylpyrrolo[3,4-c]pyrazol-5(1H,4H,6H)-yl)methanone (Intermediate TN)

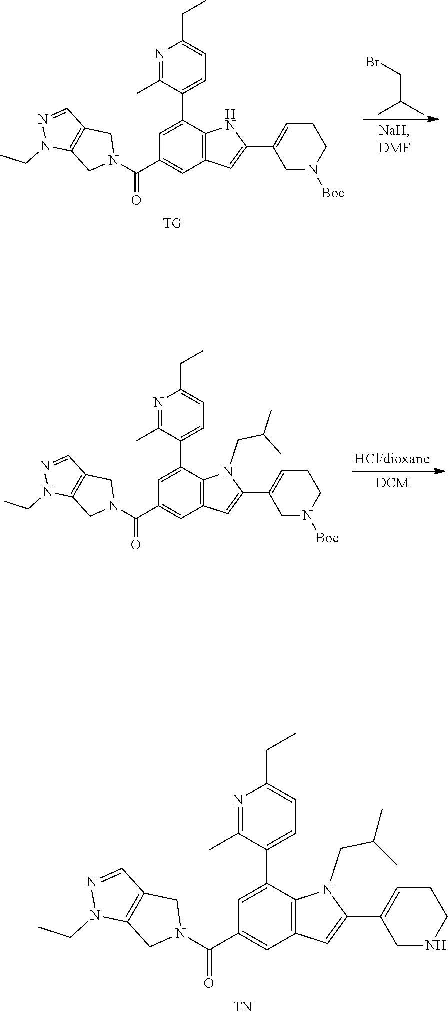

### Step 1—tert-butyl 3-(5-(1-ethyl-1,4,5,6-tetrahydropyrrolo[3,4-c]pyrazole-5-carbonyl)-7-(6-ethyl-2-methylpyridin-3-yl)-1-isobutyl-1H-indol-2-yl)-5,6-dihydropyridine-1(2H)-carboxylate

To a solution of tert-butyl 5-[5-(1-ethyl-4,6-dihydropyrrolo[3,4-c]pyrazole-5-carbonyl)-7-(6-ethyl-2-methyl-3-pyridyl)-1H-indol-2-yl]-3,6-dihydro-2H-pyridine-1-carboxylate (200.00 mg, 344.40 μmol, Intermediate TG) in DMF (3 mL) was added dropwise NaH (20.66 mg, 516.6 μmol, 60% dispersion in mineral oil) at 20° C. for 1 h. Next, -bromo-2-methyl-propane (70.78 mg, 516.6 μmol, 56.18 μL) was added dropwise at 20° C. and the mixture was stirred at 25° C. for 12 h. Upon completion, the mixture was quenched with NH₄Cl (3 mL) at 0° C., and then diluted with H₂O (3 mL) and extracted with EtOAc (10 mL×2). The combined organic layers were dried over Na₂SO₄, filtered and concentrated under reduced pressure to give a residue. The residue was purified by flash silica gel chromatography (ISCO®; 4 g SepaFlash® Silica Flash Column, Eluent of 0-80% EtOAc/Petroleum ether gradient @50 mL/min) to give the title compound (180.00 mg, 49% yield) as a yellow solid. LC-MS (ESI+) m/z 637.5 (M+H)⁺.

### Step 2—(7-(6-ethyl-2-methylpyridin-3-yl)-1-isobutyl-2-(1,2,5,6-tetrahydropyridin-3-yl)-1H-indol-5-yl)(1-ethylpyrrolo[3,4-c]pyrazol-5(1H,4H,6H)-yl)methanone

To a solution of tert-butyl 3-(5-(1-ethyl-1,4,5,6-tetrahydropyrrolo[3,4-c]pyrazole-5-carbonyl)-7-(6-ethyl-2-methylpyridin-3-yl)-1-isobutyl-1H-indol-2-yl)-5,6-dihydropyridine-1(2H)-carboxylate (180.00 mg, 169.59 μmol) in DCM (2 mL) was added HCl/dioxane (2 M, 1.44 mL) and the mixture was stirred at 20° C. for 1 h. Upon completion, the mixture was concentrated under reduced pressure to give a residue. The residue was extracted with EtOAc (5 mL×2) and washed with sodium hydrogen carbonate (5 mL). The combined organic layers were dried over Na₂SO₄, filtered and concentrated under reduced pressure to give the title compound (90.00 mg, 69% yield) as a white solid. LC-MS (ESI+) m/z 537.3 (M+H)⁺; ¹H NMR (400 MHz, DMSO-d₆) δ=7.90-7.76 (m, 1H), 7.70 (d, J=7.6 Hz, 1H), 7.30-7.16 (m, 2H), 7.09 (s, 1H), 6.55 (s, 1H), 5.95 (s, 1H), 4.78 (d, J=12.0 Hz, 2H), 4.57 (s, 2H), 4.19-3.94 (m, 4H), 3.57 (s, 4H), 2.79 (d, J=5.6 Hz, 2H), 2.17 (d, J=9.2 Hz, 3H), 1.35-1.22 (m, 7H), 0.90 (s, 3H), 0.24 (d, J=18.4 Hz, 6H).

### (1-Ethyl-7-(2-ethyl-6-methylpyridin-3-yl)-2-(1,2,5,6-tetrahydropyridin-3-yl)-1H-indol-5-yl)(4-(5-fluoro-3-methoxypyridin-2-yl)piperazin-1-yl)methanone (Intermediate TO)

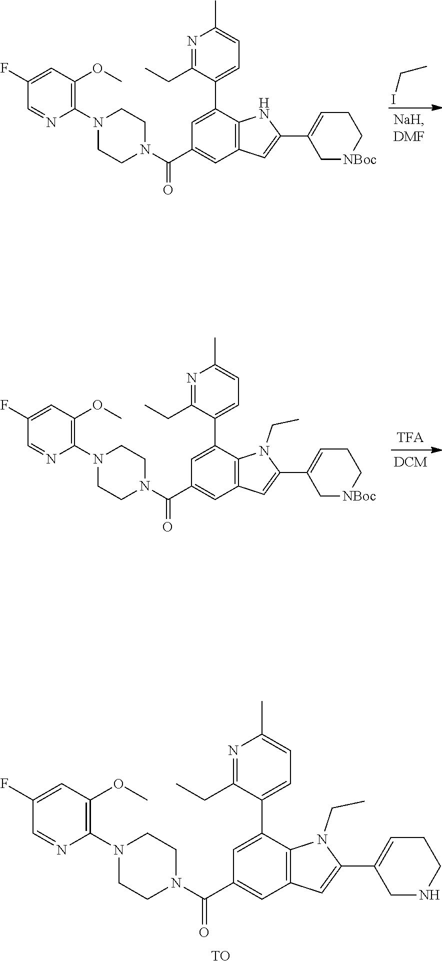

### Step 1—Tert-butyl 3-(1-ethyl-7-(2-ethyl-6-methylpyridin-3-yl)-5-(4-(5-fluoro-3-methoxypyridin-2-yl)piperazine-1-carbonyl)-1H-indol-2-yl)-5,6-dihydropyridine-1(2H)-carboxylate

To a solution of tert-butyl 5-[7-(2-ethyl-6-methyl-3-pyridyl)-5-[4-(5-fluoro-3-methoxy-2-pyridyl)piperazine-1-carbonyl]-1H-indol-2-yl]-3,6-dihydro-2H-pyridine-1-carboxylate (270 mg, 412.36 μmol, Intermediate OZ) in DMF (3 mL) was added NaH (24.7 mg, 618 μmol, 60% dispersion in mineral oil) at 0° C. and the mixture was stirred for 0.5 hr. Then, iodoethane (96.4 mg, 618 μmol) was added and the mixture was stirred at 0-25° C. for 3.5 hrs. Upon completion, the mixture was quenched with NH₄Cl (10 mL) at 0° C., and then extracted with EA (5 mL×3). The combined organic layers were washed with aqueous NaCl (8 mL×2), dried over Na₂SO₄, filtered and concentrated under reduced pressure to give a residue. The residue was purified by column chromatography (SiO₂, Petroleum ether/Ethyl acetate=1/0 to 3/2) to give the title compound (270 mg, 74% yield) as a yellow solid. LC-MS (ESI+) m/z 683.4 (M+H)⁺; ¹H NMR (400 MHz, DMSO-d₆) δ=7.78 (s, 1H), 7.71-7.65 (m, 2H), 7.35 (d, J=10.8 Hz, 1H), 7.22-7.17 (m, 1H), 6.88 (s, 1H), 6.61 (s, 1H), 6.03 (s, 1H), 4.19-4.07 (m, 1H), 4.03-3.96 (m, 1H), 3.83 (s, 3H), 3.66 (s, 4H), 3.49-3.38 (m, 4H), 3.24 (s, 4H), 2.43 (d, J=7.2 Hz, 4H), 2.27 (d, J=1.6 Hz, 3H), 1.41 (s, 9H), 1.04 (t, J=7.2 Hz, 3H), 0.67-0.61 (m, 3H).

### Step 2—(1-Ethyl-7-(2-ethyl-6-methylpyridin-3-yl)-2-(1,2,5,6-tetrahydropyridin-3-yl)-1H-indol-5-yl)(4-(5-fluoro-3-methoxypyridin-2-yl)piperazin-1-yl)methanone

To a solution of tert-butyl 5-[1-ethyl-7-(2-ethyl-6-methyl-3-pyridyl)-5-[4-(5-fluoro-3-methoxy-2-pyridyl)piperazine-1-carbonyl]indol-2-yl]-3,6-dihydro-2H-pyridine-1-carboxylate (270 mg, 395 μmol) in DCM (3 mL) was added TFA (1.54 g, 13.5 mmol). The mixture was then stirred at 25° C. for 0.5 hr. Upon completion, the mixture was concentrated under reduced pressure to give the title compound (270 mg, TFA) as a brown gum. LC-MS (ESI+) m/z 583.4 (M+H)⁺.

### 6-(1-(Tert-butoxycarbonyl)-5,5-difluoropiperidin-3-yl)-4-chloro-7-fluoro-1-((2-(trimethylsilyl)ethoxy)methyl)-1H-indole-2-carboxylic acid (Intermediate TP)

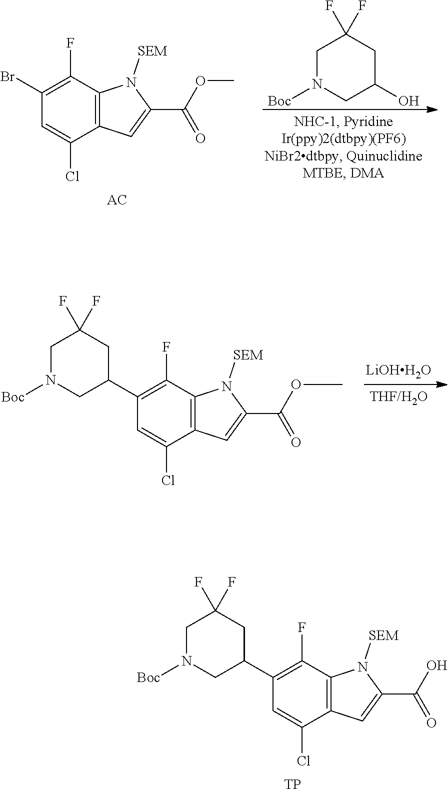

### Step 1—Methyl 6-(1-(tert-butoxycarbonyl)-5,5-difluoropiperidin-3-yl)-4-chloro-7-fluoro-1-((2-(trimethylsilyl)ethoxy)methyl)-1H-indole-2-carboxylate

To a 40 mL vial equipped with a stir bar was added tert-butyl 3,3-difluoro-5-hydroxy-piperidine-1-carboxylate (522.80 mg, 2.20 mmol, CAS #1258638-32-2), NHC-1 (796.32 mg, 2.01 mmol, CAS #1207294-92-5) in MTBE (12 mL) was added pyridine (159.37 mg, 2.01 mmol, 162.62 μL) in MTBE (1 mL) dropwise. The vial was sealed and placed under a glovebox and stirred at 25° C. for 10 min, then filtered to get the filter liquor. Next, Ir(ppy)₂(dtbpy)(PF6) (17.26 mg, 18.89 μmol), NiBr2·dtbpy (30.66 mg, 62.96 μmol), Quinuclidine (245.00 mg, 2.20 mmol) and methyl 6-bromo-4-chloro-7-fluoro-1-(2-trimethylsilylethoxymethyl)indole-2-carboxylate (550 mg, 1.26 mmol, Intermediate AC) in DMA (13 mL) was added to the above filter liquor. The reaction was then stirred and irradiated with a 10 W [455 nm] blue LED lamp (3 cm away), with cooling water to keep the reaction temperature at 25° C. for 14 h. Upon completion, the mixture was quenched with H₂O (10 mL) at 20° C., and then diluted with EtOAc (10 mL) and extracted with EtOAc (10 mL×3). The combined organic layers were washed with brine (10 mL×3), dried over Na₂SO₄, filtered and the filtrate was concentrated under reduced pressure to give the residue. The residue was purified by column chromatography (SiO₂, Petroleum ether/EtOAc=10/0 to 20/1) to give the title compound (300.00 mg, 27% yield) as a yellow oil. LC-MS (ESI⁺)/z 599.3 (M+Na)⁺.

### Step 2—6-(1-(Tert-butoxycarbonyl)-5,5-difluoropiperidin-3-yl)-4-chloro-7-fluoro-1-((2-(trimethylsilyl)ethoxy)methyl)-1H-indole-2-carboxylic acid

To a solution of methyl 6-(1-tert-butoxycarbonyl-5,5-difluoro-3-piperidyl)-4-chloro-7-fluoro-1-(2-trimethylsilylethoxymethyl)indole-2-carboxylate (50.00 mg, 86.64 μmol) in THF (1 mL) and H₂O (1 mL) was added LiOH·H₂O (18.18 mg, 433.20 μmol). The mixture was then stirred at 20° C. for 2 h. Upon completion, the mixture was quenched with HCl (0.1 mL) at 20° C., and then diluted with EtOAc (1 mL) and extracted with EtOAc (1 mL×3). The combined organic layers were washed with brine (1 mL×3), dried over Na₂SO₄, filtered and the filtrate was concentrated under reduced pressure to give the title compound (48.00 mg, 98% yield) as a brown solid. LC-MS (ESI⁺)/z 585.1 (M+Na)⁺.

### Tert-butyl 5-[4-chloro-7-fluoro-2-(1-methyl-4,6-dihydropyrrolo[3,4-c]pyrazole-5-carbonyl)-1-(2-trimethylsilylethoxymethyl)indol-6-yl]-3,3-difluoro-piperidine-1-carboxylate (Intermediate TQ)

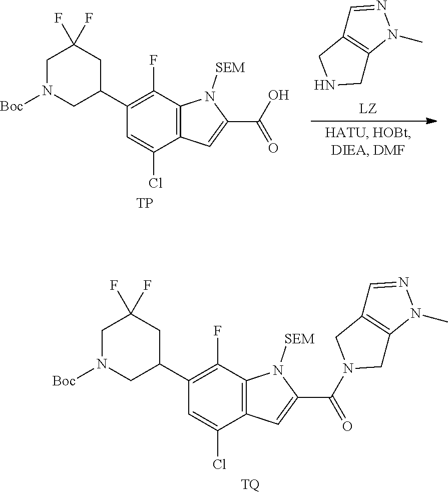

To a solution of 6-(1-tert-butoxycarbonyl-5,5-difluoro-3-piperidyl)-4-chloro-7-fluoro-1-(2-trimethylsilylethoxymethyl)indole-2-carboxylic acid (180.00 mg, 319.67 μmol, Intermediate TP), 1-methyl-5,6-dihydro-4H-pyrrolo[3,4-c]pyrazole (61.23 mg, 383.60 μmol, HCl, Intermediate LZ), HOBt (86.39 mg, 639.34 μmol), and DIEA (247.89 mg, 1.92 mmol, 334.08 μL) in DMF (2 mL) was added HATU (145.86 mg, 383.60 μmol). The reaction mixture was then stirred at 20° C. for 0.25 h. Upon completion, the mixture was poured into H₂O (10 mL) and the mixture was filtered to get the filter cake. The filter cake was triturated with H₂O (6 mL) at 20° C. for 10 min to give the title compound (180.00 mg, 78% yield) as a white solid. LC-MS (ESI⁺)/z 668.3 (M+H)⁺.

### [6-(5,5-Difluoro-3-piperidyl)-7-fluoro-4-(2-methoxyphenyl)-1H-indol-2-yl]-(1-methyl-4,6-dihydropyrrolo[3,4-c]pyrazol-5-yl)methanone (Intermediate TR)

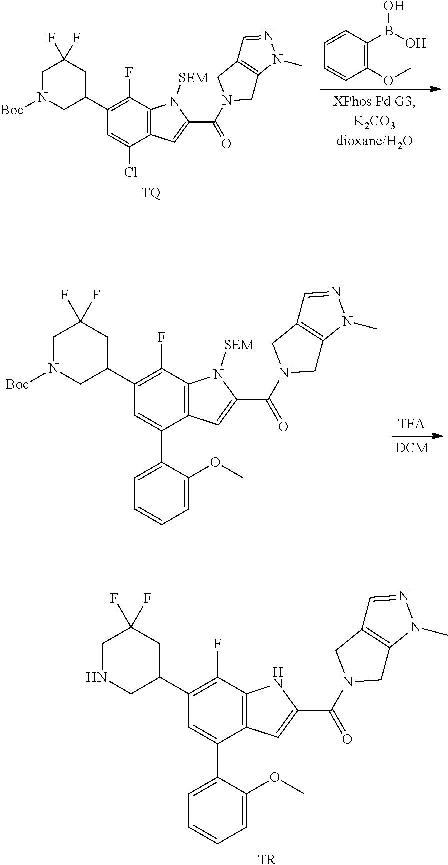

### Step 1—Tert-butyl 3,3-difluoro-5-[7-fluoro-4-(2-methoxyphenyl)-2-(1-methyl-4,6-dihydropyrrolo[3,4-c]pyrazole-5-carbonyl)-1-(2-trimethylsilylethoxymethyl)indol-6-yl]piperidine-1-carboxylate

To a solution of tert-butyl 5-[4-chloro-7-fluoro-2-(1-methyl-4,6-dihydropyrrolo[3,4-c]pyrazole-5-carbonyl)-1-(2-trimethylsilylethoxymethyl)indol-6-yl]-3,3-difluoro-piperidine-1-carboxylate (180.00 mg, 250.52 μmol, Intermediate TQ) and (2-methoxyphenyl)boronic acid (57.10 mg, 375.8 μmol, CAS #5720-06-9), and K₂CO₃ (103.87 mg, 751.55 μmol) in dioxane (3 mL) and H₂O (0.6 mL) was added XPhos Pd G₃ (21.20 mg, 25.05 μmol) under N₂. The mixture was then stirred at 80° C. for 12 h under N₂ atmosphere. Upon completion, the mixture was quenched with H₂O (3 mL) at 20° C., and extracted with DCM (5 mL×3). The combined organic layers were dried over Na₂SO₄, filtered and the filtrate was concentrated under reduced pressure to give a residue. The residue was purified by flash silica gel chromatography (ISCO®; 12 g SepaFlash® Silica Flash Column, Eluent of 0˜20% EtOAc/Petroleum ether gradient @70 mL/min) to give the title compound (115.00 mg, 59% yield) as a yellow solid. H NMR (400 MHz, CDCl₃-d) δ=7.48-7.31 (m, 2H), 7.13-6.96 (m, 3H), 6.75-6.58 (m, 1H), 5.95-5.80 (m, 2H), 4.87-4.58 (m, 4H), 3.96-3.66 (m, 7H), 3.61-3.43 (m, 4H), 1.62-1.44 (m, 14H), 0.91-0.68 (m, 2H), −0.09-−0.17 (m, 9H).

### Step 2—[6-(5,5-Difluoro-3-piperidyl)-7-fluoro-4-(2-methoxyphenyl)-1H-indol-2-yl]-(1-methyl-4,6-dihydropyrrolo[3,4-c]pyrazol-5-yl)methanone

A mixture of tert-butyl 3,3-difluoro-5-[7-fluoro-4-(2-methoxyphenyl)-2-(1-methyl-4,6-dihydropyrrolo[3,4-c]pyrazole-5-carbonyl)-1-(2-trimethylsilylethoxymethyl)indol-6-yl]piperidine-1-carboxylate (115.00 mg, 147.66 μmol) and TFA (1.54 g, 13.5 mmol, 1 mL) in DCM (2 mL) was stirred at 20° C. for 12 h. Upon completion, the mixture was quenched with H₂O (8 mL) and the mixture's value of pH was adjusted to 7 by adding aqueous NaHCO₃. The mixture was then extracted with DCM (5 mL×3). The combined organic layers were dried over Na₂SO₄, filtered and the filtrate was concentrated under reduced pressure to give the title compound (75.00 mg, 99% yield) as a light yellow gum. LC-MS (ESI⁺)/z 510.2 (M+H)⁺.

### Tert-butyl 5-(4-chloro-7-fluoro-2-(4-(5-fluoro-3-methoxypyridin-2-yl)piperazine-1-carbonyl)-1-((2-(trimethylsilyl)ethoxy)methyl)-1H-indol-6-yl)-3,3-difluoropiperidine-1-carboxylate (Intermediate TS)

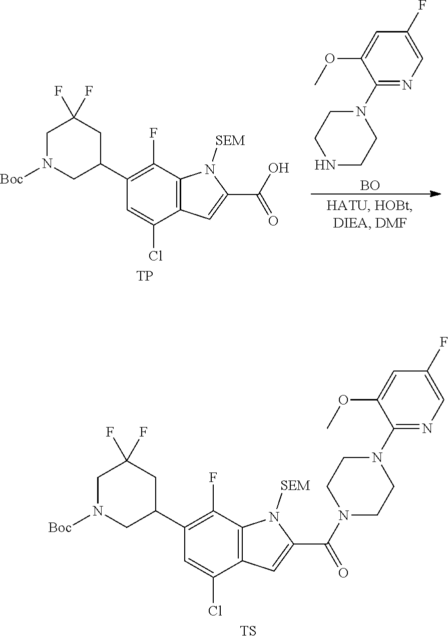

To a solution of 6-(1-(tert-butoxycarbonyl)-5,5-difluoropiperidin-3-yl)-4-chloro-7-fluoro-1-((2-(trimethylsilyl)ethoxy)methyl)-1H-indole-2-carboxylic acid (48.00 mg, 85.25 μmol, Intermediate TP) and 1-(5-fluoro-3-methoxypyridin-2-yl)piperazine (21.61 mg, 102.3 μmol, Intermediate BO) in DMF (1 mL) was added HATU (38.90 mg, 102.29 μmol), HOBt (23.04 mg, 170.49 μmol) and DIEA (66.10 mg, 511.47 μmol, 89.09 μL). The mixture was then stirred at 25° C. for 0.5 h. Upon completion, the mixture was quenched with H₂O (2 mL) at 20° C., and then diluted with EtOAc (2 mL) and extracted with EtOAc (2 mL×3). The combined organic layers were washed with brine (2 mL×3), dried over Na₂SO₄, filtered and the filtrate was concentrated under reduced pressure to give the title compound (60.00 mg, 82% yield) as yellow solid. ¹H NMR (400 MHz, CDCl₃-d) δ=8.02 (s, 2H), 7.76 (d, J=2.4 Hz, 1H), 6.97-6.89 (m, 2H), 5.77 (s, 2H), 4.04-3.72 (m, 9H), 3.52 (t, J=8.0 Hz, 2H), 3.43-3.35 (m, 4H), 2.81 (s, 2H), 1.54-1.44 (m, 11H), 0.89-0.85 (m, 2H), −0.06 (s, 9H).

### (6-(5,5-Difluoropiperidin-3-yl)-7-fluoro-4-(4-methoxypyridin-3-yl)-1H-indol-2-yl)(4-(5-fluoro-3-methoxypyridin-2-yl)piperazin-1-yl)methanone (Intermediate TT)

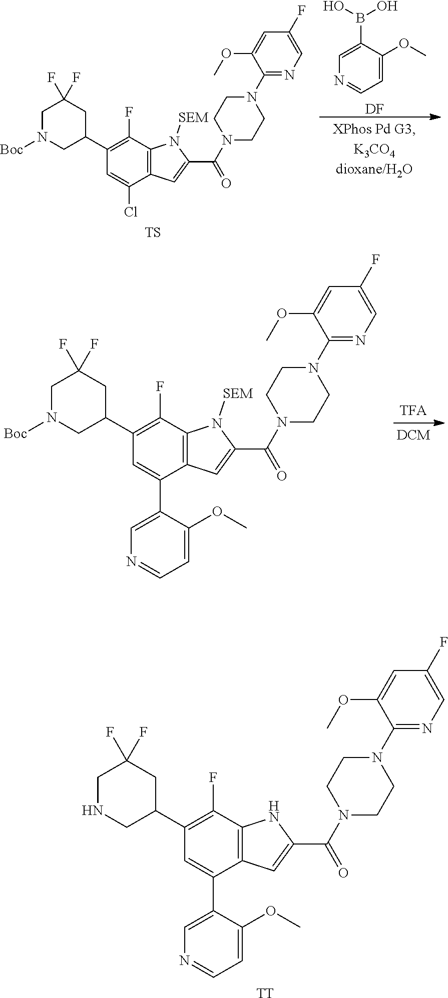

### Step 1—Tert-butyl 3,3-difluoro-5-(7-fluoro-2-(4-(5-fluoro-3-methoxypyridin-yl)piperazine-1-carbonyl)-4-(4-methoxypyridin-3-yl)-1-((2-(trimethylsilyl)ethoxy)methyl)-1H-indol-6-yl)piperidine-1-carboxylate

To a solution of tert-butyl 5-(4-chloro-7-fluoro-2-(4-(5-fluoro-3-methoxypyridin-2-yl)piperazine-1-carbonyl)-1-((2-(trimethylsilyl)ethoxy)methyl)-1H-indol-6-yl)-3,3-difluoropiperidine-1-carboxylate (60.00 mg, 79.33 μmol, Intermediate TS) and (4-methoxypyridin-3-yl)boronic acid (18.20 mg, 119.00 μmol, Intermediate DF) in dioxane (2 mL) and H₂O (0.5 mL) were added XPhos Pd G₃ (6.72 mg, 7.93 μmol) and K₂CO₃ (32.89 mg, 238.00 μmol). The mixture was then stirred at 80° C. for 2 h. Upon completion, the mixture was diluted with H₂O (3 mL) and extracted with EtOAc (3 mL×3). The combined organic layers were washed with brine (3 mL×3), dried over Na₂SO₄, filtered and the filtrate was concentrated under reduced pressure to give a residue. The residue was purified by prep-TLC (SiO₂, DCM: MeOH=10:1) to give the title compound (50.00 mg, 71% yield) as a white solid. LC-MS (ESI⁺)/z 829.4 (M+H)⁺.

### Step 2—(6-(5,5-Difluoropiperidin-3-yl)-7-fluoro-4-(4-methoxypyridin-3-yl)-1H-indol-2-yl)(4-(5-fluoro-3-methoxypyridin-2-yl)piperazin-1-yl)methanone

To a solution of tert-butyl 3,3-difluoro-5-[7-fluoro-2-[4-(5-fluoro-3-methoxy-2-pyridyl)piperazine-1-carbonyl]-4-(4-methoxy-3-pyridyl)-1-(2-trimethylsilylethoxymethyl)indol-6-yl]piperidine-1-carboxylate (40.00 mg, 48.25 μmol) in DCM (0.4 mL) was added TFA (5.50 mg, 48.25 gmol, 3.58 μL). The mixture was then stirred at 20° C. for 24 h. Upon completion, the mixture was quenched with NaHCO₃ (1 mL) at 25° C., and then diluted with DCM (1 mL) and extracted with DCM (1 mL×3). The combined organic layers were washed with brine (1 mL×3), dried over Na₂SO₄, filtered and the filtrate was concentrated under reduced pressure to give a residue. The residue was purified by prep-TLC (SiO₂, DCM:MeOH=5:1) to give the title compound (20.00 mg, 69% yield) as a yellow oil. LC-MS (ESI⁺)/z 599.2 (M+H)⁺.

### (4-(4-Ethylpyridin-3-yl)-7-fluoro-6-(1,2,5,6-tetrahydropyridin-3-yl)-1H-indol-2-yl)(4-(5-fluoro-3-methoxypyridin-2-yl)piperazin-1-yl)methanone (Intermediate TU)

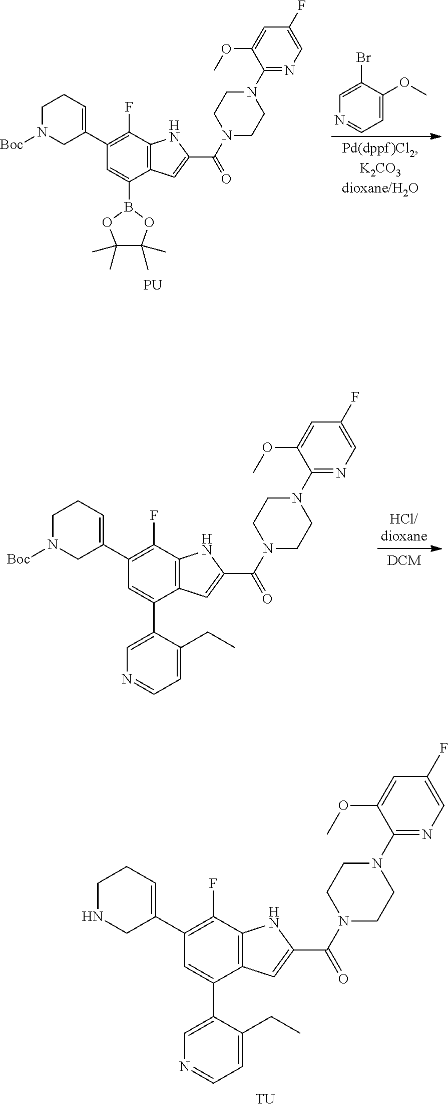

### Step 1—Tert-butyl 3-(4-(4-ethylpyridin-3-yl)-7-fluoro-2-(4-(5-fluoro-3-methoxypyridin-2-yl)piperazine-1-carbonyl)-1H-indol-6-yl)-5,6-dihydropyridine-1(2H)-carboxylate

To a solution of tert-butyl 5-[7-fluoro-2-[4-(5-fluoro-3-methoxy-2-pyridyl)piperazine-1-carbonyl]-4-(4,4,5,5-tetramethyl-1,3,2-dioxaborolan-2-yl)-1H-indol-6-yl]-3,6-dihydro-2H-pyridine-1-carboxylate (1.36 g, 2.00 mmol, Intermediate PU), 3-bromo-4-ethyl-pyridine (409.57 mg, 2.20 mmol, CAS #38749-76-7), K₂CO₃ (829.80 mg, 6.00 mmol) in dioxane (15 mL) and H₂O (3 mL) was added Pd(dppf)Cl₂ (146.43 mg, 200.13 μmol) under N₂. The reaction mixture was then stirred at 80° C. for 1 h. Upon completion, the mixture was quenched with H₂O (15 mL) at 20° C., and then extracted with DCM (15 mL×3). The combined organic layers were dried over Na₂SO₄, filtered and the filtrate was concentrated under reduced pressure to give a residue. The residue was purified by flash silica gel chromatography (ISCO®; 20 g SepaFlash® Silica Flash Column, Eluent of 0˜30% EtOAc/Petroleum ether gradient @80 mL/min) to give the title compound (930.00 mg, 67% yield) as a yellow solid. ¹H NMR (400 MHz, DMSO-d₆) δ 12.32 (s, 1H), 8.52 (d, J=4.8 Hz, 1H), 8.41 (s, 1H), 7.77 (d, J=2.0 Hz, 1H), 7.42 (d, J=5.2 Hz, 1H), 7.35 (dd, J=2.4, 10.4 Hz, 1H), 6.88 (d, J=6.0 Hz, 1H), 6.35 (s, 1H), 6.15 (s, 1H), 4.24 (s, 2H), 3.83 (s, 3H), 3.76 (s, 4H), 3.50 (t, J=5.2 Hz, 2H), 3.23 (s, 4H), 2.54 (s, 2H), 2.28 (d, J=2.8 Hz, 2H), 1.42 (s, 9H), 1.01 (t, J=7.2 Hz, 3H).

### Step 2—(4-(4-Ethylpyridin-3-yl)-7-fluoro-6-(1,2,5,6-tetrahydropyridin-3-yl)-1H-indol-2-yl)(4-(5-fluoro-3-methoxypyridin-2-yl)piperazin-1-yl)methanone

A solution of tert-butyl 5-[4-(4-ethyl-3-pyridyl)-7-fluoro-2-[4-(5-fluoro-3-methoxy-2-pyridyl)piperazine-1-carbonyl]-1H-indol-6-yl]-3,6-dihydro-2H-pyridine-1-carboxylate (930.00 mg, 1.41 mmol) and HCl/dioxane (2 M, 10 mL) in DCM (10 mL) was stirred at 20° C. for 12 h. Upon completion, the mixture was concentrated under reduced pressure to give the title compound (800.00 mg, 95% yield, HCl) as a yellow solid. ¹H NMR (400 MHz, DMSO-d₆) δ 12.55 (s, 1H), 9.69 (s, 2H), 8.92 (d, J=6.0 Hz, 1H), 8.83 (s, 1H), 8.14 (d, J=6.0 Hz, 1H), 7.78 (d, J=2.0 Hz, 1H), 7.37 (dd, J=2.0, 10.0 Hz, 1H), 7.13 (d, J=6.0 Hz, 1H), 6.59 (s, 1H), 6.31 (s, 1H), 4.15-4.11 (m, 1H), 3.98 (s, 2H), 3.83 (s, 3H), 3.76 (s, 4H), 3.25 (s, 6H), 2.77-2.64 (m, 2H), 1.08 (t, J=7.6 Hz, 3H).

### (S)-methyl 6-(1-(3-(1H-1,2,3-triazol-1-yl)propanoyl)-1,2,5,6-tetrahydropyridin-3-yl)-7-fluoro-4-(1-methylpyrrolidin-3-yl)-1H-indole-2-carboxylate (Intermediate TV) & (R)-methyl 6-(1-(3-(1H-1,2,3-triazol-1-yl)propanoyl)-1,2,5,6-tetrahydropyridin-3-yl)-7-fluoro-4-(1-methylpyrrolidin-3-yl)-1H-indole-2-carboxylate (Intermediate TW)

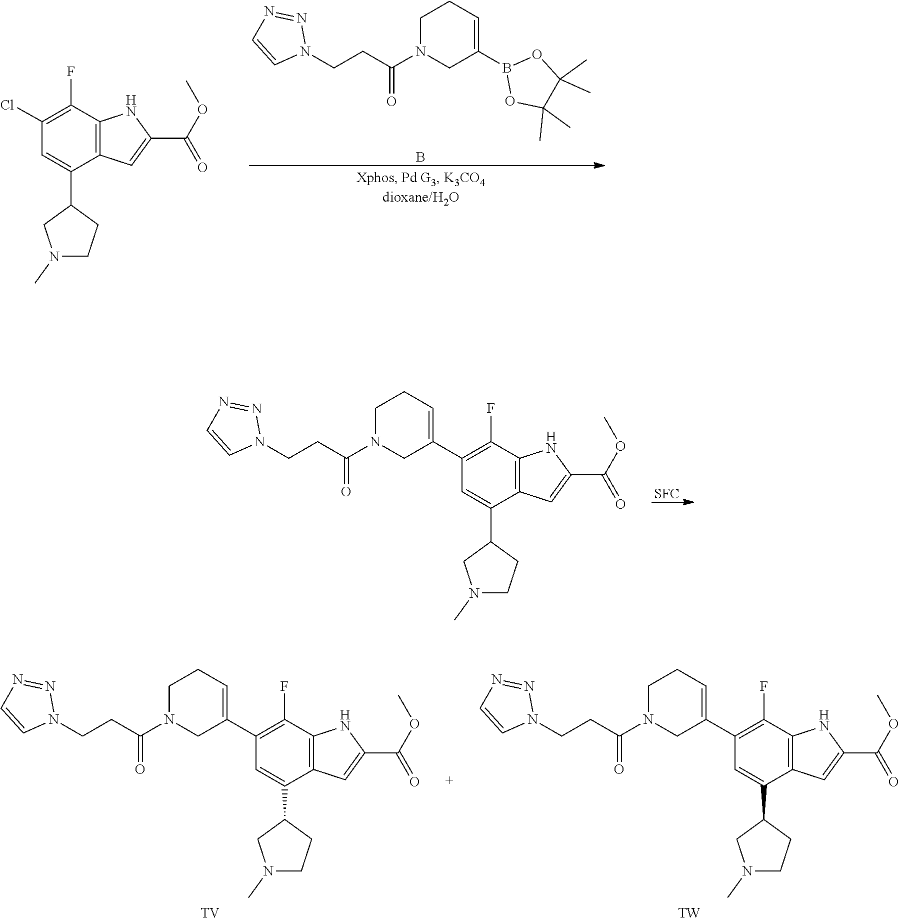

### Step 1—Methyl 6-(1-(3-(1H-1,2,3-triazol-1-yl)propanoyl)-1,2,5,6-tetrahydropyridin-3-yl)-7-fluoro-4-(1-methylpyrrolidin-3-yl)-1H-indole-2-carboxylate

A mixture of methyl 6-chloro-7-fluoro-4-(1-methylpyrrolidin-3-yl)-1H-indole-2-carboxylate (336 mg, 1.08 mmol, synthesized via Steps 1-4 of Intermediate GR), 1-(3-(4,4,5,5-tetramethyl-1,3,2-dioxaborolan-2-yl)-5,6-dihydropyridin-1(2H)-yl)-3-(1H-1,2,3-triazol-1-yl)propan-1-one (359 mg, 1.08 mmol, Intermediate B), XPhos Pd G₃ (91.5 mg, 108 μmol), and Na₂CO₃ (343.8 mg, 3.24 mmol) in dioxane (8 mL) and H₂O (0.8 mL) was degassed and purged with N₂ three times. Then the mixture was stirred at 80° C. for 2 hr under N₂ atmosphere. The mixture was diluted with water (50 mL) and extracted with ethyl acetate (50 mL×2), dried over sodium sulfate, filtered and concentrated under reduced pressure to give a residue. The residue was purified by silica gel column chromatography (petroleum ether/ethyl acetate=20/1) to give the title compound (465 mg, 83% yield) as a white solid. LC-MS (ESI⁺) m/z 481.2 (M+H)⁺.

### Step 2—(S)-methyl 6-(1-(3-(1H-1,2,3-triazol-1-yl)propanoyl)-1,2,5,6-tetrahydropyridin-3-yl)-7-fluoro-4-(1-methylpyrrolidin-3-yl)-1H-indole-2-carboxylate & (R)-methyl 6-(1-(3-(1H-1,2,3-triazol-1-yl)propanoyl)-1,2,5,6-tetrahydropyridin-3-yl)-7-fluoro-4-(1-methylpyrrolidin-3-yl)-1H-indole-2-carboxylate

Methyl 6-(1-(3-(1H-1,2,3-triazol-1-yl)propanoyl)-1,2,5,6-tetrahydropyridin-3-yl)-7-fluoro-4-(1-methylpyrrolidin-3-yl)-1H-indole-2-carboxylate was separated by SFC (column: DAICEL CHIRALPAK AD(250 mm*30 mm, 10 um);mobile phase: [CO₂-ACN/i-PrOH(0.1% NH₃H₂O)];B %:60%, isocratic elution mode) to give the first eluting peak (S)-methyl 6-(1-(3-(1H-1,2,3-triazol-1-yl)propanoyl)-1,2,5,6-tetrahydropyridin-3-yl)-7-fluoro-4-(1-methylpyrrolidin-3-yl)-1H-indole-2-carboxylate (200 mg, 39% yield) as a white solid (LC-MS (ESI⁺) m/z 481.1 (M+H)⁺) and the second eluting peak (R)-methyl 6-(1-(3-(1H-1,2,3-triazol-1-yl)propanoyl)-1,2,5,6-tetrahydropyridin-3-yl)-7-fluoro-4-(1-methylpyrrolidin-3-yl)-1H-indole-2-carboxylate (200 mg, 38% yield) as a white solid (LC-MS (ESI⁺) m/z 481.2 (M+H)⁺). The absolute configuration of the enantiomers was assigned arbitrarily.

### (S)-6-(1-(3-(1H-1,2,3-triazol-1-yl)propanoyl)-1,2,5,6-tetrahydropyridin-3-yl)-7-fluoro-4-(1-methylpyrrolidin-3-yl)-1H-indole-2-carboxylic acid (Intermediate TX)

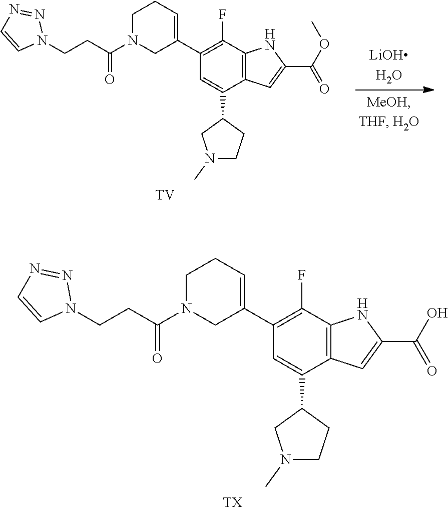

A mixture of methyl 7-fluoro-4-[(3S)-1-methylpyrrolidin-3-yl]-6-[1-[3-(triazol-1-yl)propanoyl]-3,6-dihydro-2H-pyridin-5-yl]-1H-indole-2-carboxylate (198 mg, 412 μmol, Intermediate TV) and LiOH·H₂O (519 mg, 1.24 mmol) in THF (4 mL), MeOH (1 mL) and H₂O (1 mL) was degassed and purged with N₂ three times then the mixture was stirred at 25° C. for 12 hrs under N₂ atmosphere. Upon completion, the mixture was diluted with water (10 mL) and extracted with ethyl acetate (50 mL×3), dried over sodium sulfate, filtered and concentrated under reduced pressure to give the title compound (175 mg, 61% yield) as a white solid. LC-MS (ESI⁺) m/z 467.1 (M+H)⁺.

### Example I-331 (Method 1): Synthesis of 1-(5-(7-fluoro-4-(2-methoxyphenyl)-2-(4-(3-(trifluoromethoxy)pyridin-2-yl)piperazine-1-carbonyl)-1H-indol-6-yl)-3,6-dihydropyridin-1(2H)-yl)-3-(1H-1,2,3-triazol-1-yl)propan-1-one

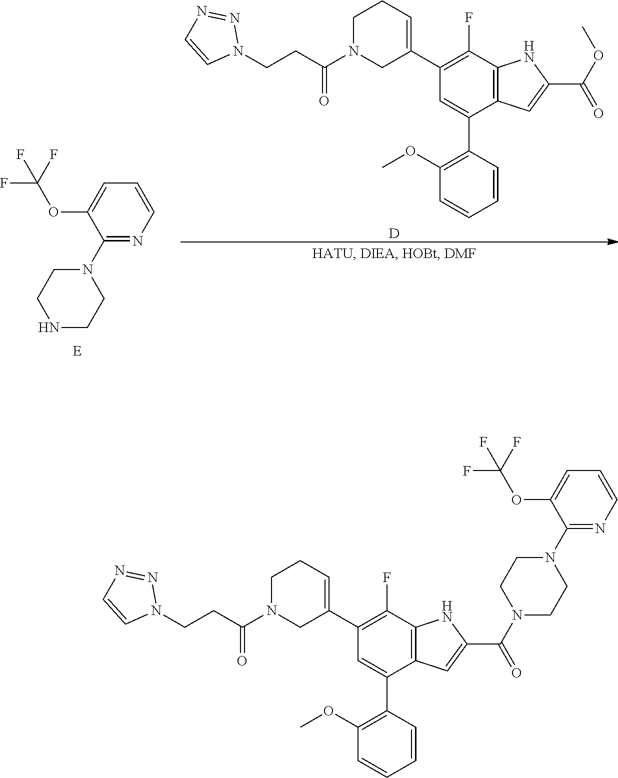

To a solution of 6-(1-(3-(1H-1,2,3-triazol-1-yl)propanoyl)-1,2,5,6-tetrahydropyridin-3-yl)-7-fluoro-4-(2-methoxyphenyl)-1H-indole-2-carboxylic acid (70 mg, 143 μmol, Intermediate D) in DMF (1 mL) was added HATU (65.3 mg, 172 μmol), DIEA (92.4 mg, 715 μmol, 125 μL) and HOBt (29.0 mg, 215 μmol) at 25° C. Then 1-(3-(trifluoromethoxy)pyridin-2-yl)piperazine (82.3 mg, 215 μmol, HCl, Intermediate E) was added at 25° C., and the mixture was stirred at 25° C. for 20 min. On completion, the reaction mixture was concentrated in vacuo to get the crude residue. The crude product was purified by reversed-phase HPLC (0.1% FA condition) to give the title compound (58.3 mg, 53% yield, FA) as a yellow solid. LC-MS (ESI⁺) m/z 719.1 (M+H)⁺; ¹H NMR (400 MHz, DMSO-d₆) δ=12.12 (d, J=7.6 Hz, 1H), 8.22 (d, J=4.4 Hz, 1H), 8.14-8.07 (m, 1H), 7.73-7.65 (m, 2H), 7.41-7.33 (m, 2H), 7.15 (d, J=8.4 Hz, 1H), 7.07-6.99 (m, 2H), 6.98-6.89 (m, 1H), 6.46 (s, 1H), 6.17-6.09 (m, 1H), 4.65-4.59 (m, 2H), 4.39-4.30 (m, 2H), 3.80 (s, 4H), 3.73 (s, 3H), 3.65 (t, J=5.2 Hz, 1H), 3.61-3.58 (m, 1H), 3.43 (s, 4H), 3.13-3.06 (m, 2H), 2.37-2.25 (m, 2H).

### Tert-butyl 5-[7-bromo-5-(4-cyclopropylpiperazine-1-carbonyl)-1H-indol-2-yl]-3,6-dihydro-2H-pyridine-1-carboxylate (Intermediate TY)

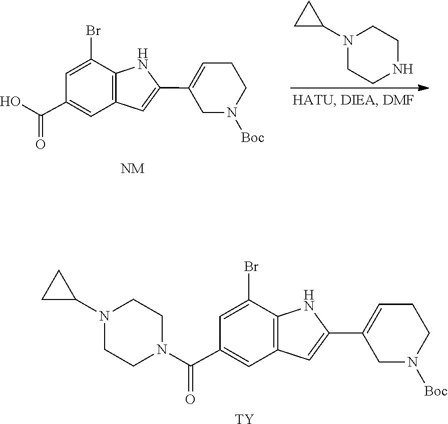

To a solution of 7-bromo-2-(1-tert-butoxycarbonyl-3,6-dihydro-2H-pyridin-5-yl)-1H-indole-5-carboxylic acid (1 g, 2.4 mmol, Intermediate NM) in DMF (10 mL) was added DIEA (920 mg, 7.12 mmol, 1.24 mL) and HATU (1.35 g, 3.56 mmol) and the reaction was stirred at 25° C. for 0.2 hr. Then 1-cyclopropylpiperazine (299 mg, 2.37 mmol, CAS #139256-79-4) was added to above mixture and the mixture was stirred at 25° C. for 0.5 hr. On completion, the reaction mixture was diluted with water (40 mL) and extracted with EA (20 mL×2). The combined organic layers were washed with saturated brine (50 mL). The organic layer was dried over Na₂SO₄, filtered and concentrated in vacuo to give the title compound (1.2 g, 93% purity) as yellow solid. LC-MS (ESI⁺) m/z 529.2 (M+H)⁺; ¹H NMR (400 MHz, DMSO-d₆) δ 11.25 (s, 1H), 7.57 (s, 1H), 7.31 (d, J=1.2 Hz, 1H), 6.82 (s, 1H), 6.66-6.54 (m, 1H), 4.25 (s, 2H), 3.50 (t, J=5.6 Hz, 6H), 2.90 (s, 1H), 2.74 (s, 1H), 2.70 (s, 1H), 2.32 (s, 2H), 1.70-1.63 (m, 1H), 1.45 (s, 9H), 1.29-1.23 (m, 1H), 0.47-0.41 (m, 2H), 0.34 (d, J=2.8 Hz, 2H).

### Tert-butyl 5-[1-(cyclopropylmethyl)-5-(4-cyclopropylpiperazine-1-carbonyl)-7-(4,4,5,5-tetramethyl-1,3,2-dioxaborolan-2-yl)indol-2-yl]-3,6-dihydro-2H-pyridine-1-carboxylate (Intermediate TZ)

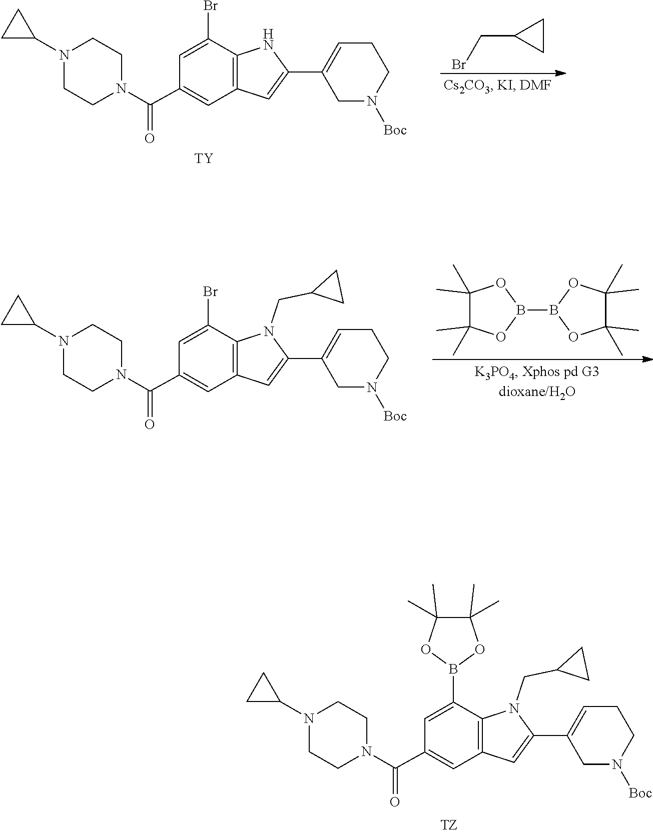

### Step 1—Tert-butyl 5-[7-bromo-1-(cyclopropylmethyl)-5-(4-cyclopropylpiperazine-1-carbonyl)indol-2-yl]-3,6-dihydro-2H-pyridine-1-carboxylate

To a solution of tert-butyl 5-[7-bromo-5-(4-cyclopropylpiperazine-1-carbonyl)-1H-indol-2-yl]-3,6-dihydro-2H-pyridine-1-carboxylate (1.1 g, 2.1 mmol, Intermediate TY) and bromomethylcyclopropane (420 mg, 3.12 mmol) in DMF (10 mL) was added Cs₂CO₃ (2.03 g, 6.23 mmol) and KI (34.4 mg, 207 μmol), and the reaction was stirred at 25° C. for 3 hrs. On completion, the reaction mixture was diluted with EA (50 mL) and washed with water (50 mL×4), and the organic layer was dried over Na₂SO₄, filtered and concentrated in vacuo. The crude product was purified by column chromatography (SiO₂, PE:EA=50:1 to 0:1, Rf=0.5) to give the title compound (1.1 g) as yellow solid. LC-MS (ESI⁺) m/z 584.9 (M+H)⁺; ¹H NMR (400 MHz, DMSO-d₆) δ 7.64-7.58 (m, 1H), 7.37 (d, J=0.8 Hz, 1H), 6.64-6.51 (m, 1H), 6.06 (br s, 1H), 4.45 (br d, J=6.8 Hz, 2H), 4.08 (br s, 2H), 3.51 (br t, J=5.2 Hz, 6H), 2.54 (s, 4H), 2.30 (br s, 2H), 1.66 (tt, J=3.2, 6.4 Hz, 1H), 1.43 (s, 9H), 1.11-0.99 (m, 1H), 0.45-0.40 (m, 2H), 0.39-0.30 (m, 4H), 0.18 (q, J=5.0 Hz, 2H).

### Step 2—Tert-butyl 5-[1-(cyclopropylmethyl)-5-(4-cyclopropylpiperazine-1-carbonyl)-7-(4,4,5,5-tetramethyl-1,3,2-dioxaborolan-2-yl)indol-2-yl]-3,6-dihydro-2H-pyridine-1-carboxylate

To a solution of tert-butyl 5-[7-bromo-1-(cyclopropylmethyl)-5-(4-cyclopropylpiperazine-1-carbonyl) indol-2-yl]-3,6-dihydro-2H-pyridine-1-carboxylate (900 mg, 1.54 mmol) and 4,4,5,5-tetramethyl-2-(4,4,5,5-tetramethyl-1,3,2-dioxaborolan-2-yl)-1,3,2-dioxaborolane (1.96 g, 7.71 mmol, CAS #73183-34-3) in dioxane (9 mL) and H₂O (1.8 mL) was added XPhos Pd G₃ (130 mg, 154 μmol) and K₃PO₄ (982 mg, 4.63 mmol). Then the reaction was stirred at 80° C. for 1 hr under N₂. On completion, the reaction mixture was diluted with water (40 mL) and extracted with EA (20 mL×2). The combined organic layers were washed with saturated brine (50 mL), and the organic layer was dried over Na₂SO₄, filtered and concentrated in vacuo. The crude product was purified by column chromatography (SiO₂, PE:EA=10:1 to 1:1, Rf=0.5) to give the title compound (750 mg) as yellow solid. LC-MS (ESI⁺) m/z 631.4 (M+H)⁺.

### [1—(Cyclopropylmethyl)-7-(2-ethyl-6-methyl-3-pyridyl)-2-(1,2,3,6-tetrahydropyridin-5-yl)indol-5-yl]-(4-cyclopropylpiperazin-1-yl)methanone (Intermediate UA)

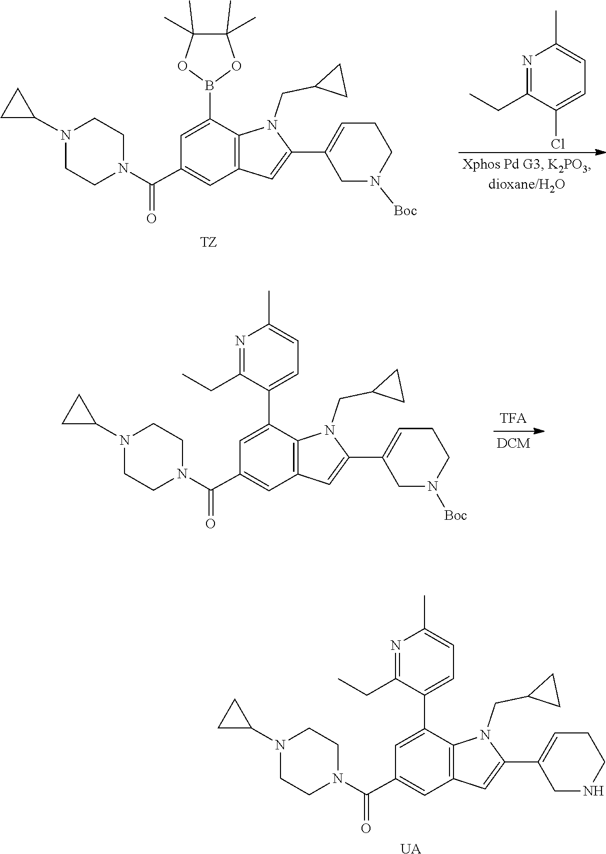

### Step 1—Tert-butyl 5-[1-(cyclopropylmethyl)-5-(4-cyclopropylpiperazine-1-carbonyl)-7-(2-ethyl-6-methyl-3-pyridyl)indol-2-yl]-3,6-dihydro-2H-pyridine-1-carboxylate

To a solution of tert-butyl 5-[1-(cyclopropylmethyl)-5-(4-cyclopropylpiperazine-1-carbonyl)-7-(4,4,5,5-tetramethyl-1,3,2-dioxaborolan-2-yl)indol-2-yl]-3,6-dihydro-2H-pyridine-1-carboxylate (700 mg, 1.11 mmol, Intermediate TZ) and 3-chloro-2-ethyl-6-methyl-pyridine (172 mg, 1.11 mmol) in dioxane (9 mL) and H₂O (1.8 mL) was added XPhos Pd G₃ (93.9 mg, 111 μmol) and K₂CO₃ (460 mg, 3.33 mmol), and the reaction was stirred at 80° C. for 3 hrs under N₂. On completion, the reaction mixture was diluted with water (100 mL) and extracted with EA (50 mL×2). The combined organic layers were washed with saturated brine (50 mL), dried over Na₂SO₄, filtered and concentrated in vacuo. The crude product was purified by column chromatography (SiO₂, PE:EA=10:1 to 0:1, PE: EA=0:1, Rf=0.3) and purified by reverse phase (0.1% FA) to give the title compound (102 mg) as yellow solid. LC-MS (ESI⁺) m/z 624.5 (M+H)⁺; ¹H NMR (400 MHz, DMSO-d₆) δ 7.72-7.62 (m, 2H), 7.21 (d, J=7.8 Hz, 1H), 6.86 (d, J=1.6 Hz, 1H), 6.63 (s, 1H), 5.97 (s, 1H), 3.50-3.48 (m, 2H), 3.46-3.41 (m, 12H), 2.55 (s, 5H), 2.25 (d, J=1.6 Hz, 2H), 1.41 (s, 10H), 1.05-1.00 (m, 3H), 0.52-0.45 (m, 1H), 0.42 (d, J=4.4 Hz, 2H), 0.32 (d, J=3.2 Hz, 2H), 0.10 (dd, J=5.6, 7.6 Hz, 2H), −0.22 (d, J=4.4 Hz, 2H).

### Step 2—[1—(Cyclopropylmethyl)-7-(2-ethyl-6-methyl-3-pyridyl)-2-(12,3,6-tetrahydropyridin-5-yl)indol-5-yl]-(4-cyclopropylpiperazin-1-yl)methanone

A solution of tert-butyl 5-[1-(cyclopropylmethyl)-5-(4-cyclopropylpiperazine-1-carbonyl)-7-(2-ethyl-6-methyl-3-pyridyl)indol-2-yl]-3,6-dihydro-2H-pyridine-1-carboxylate (90 mg, 144 mol) in DCM (1 mL) and TFA (0.2 mL) was stirred at 25° C. for 0.2 hr. On completion, the reaction was concentrated in vacuo to give the title compound (90 mg, TFA) as yellow oil. LC-MS (ESI⁺) m/z 524.3 (M+H)⁺.

### Methyl 2-bromo-7-chlorobenzo[b]thiophene-5-carboxylate (Intermediate UB)

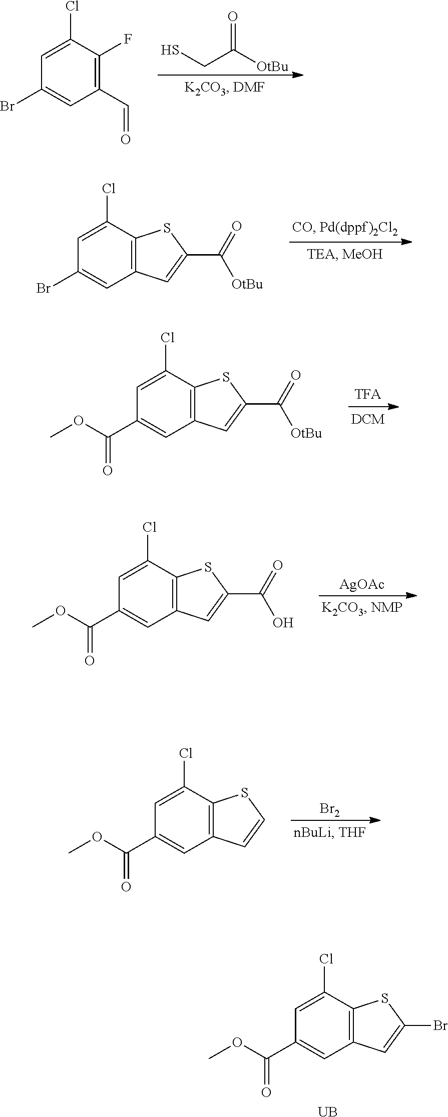

### Step 1—Tert-butyl 5-bromo-7-chlorobenzo[b]thiophene-2-carboxylate

To a solution of 5-bromo-3-chloro-2-fluoro-benzaldehyde (10 g, 40 mmol, CAS #1280786-80-2) and tert-butyl 2-sulfanylacetate (6.24 g, 42.1 mmol, CAS #20291-99-0) in DMF (100 mL) was added K₂CO₃ (11.64 g, 84.2 mmol) at 20° C. The mixture was then stirred at 80° C. for 2 hrs. On completion, the reaction mixture was diluted with water (500 mL) and extracted with ethyl acetate (3×200 mL). The combined organic layers were washed with brine (100 mL) and dried over anhydrous sodium sulfate, filtered and the filtrate was concentrated in vacuo to give the title compound (13 g) as a green solid. ¹H NMR (400 MHz, CDCl₃-d) δ=7.89-7.88 (m, 2H), 7.55 (d, J=1.5 Hz, 1H), 1.62 (s, 9H).

### Step 2—2-Tert-butyl 5-methyl 7-chlorobenzo[b]thiophene-2,5-dicarboxylate

To a solution of tert-butyl 5-bromo-7-chloro-benzothiophene-2-carboxylate (11 g, 32 mmol,) in MeOH (50 mL) was added TEA (12.81 g, 126.6 mmol, 17.62 mL) and Pd(dppf)₂Cl₂ (2.32 g, 3.16 mmol). The suspension was degassed and purged with CO three times. Then the mixture was stirred under CO (50 Psi) at 80° C. for 12 hrs. On completion, the reaction mixture was concentrated under reduced pressure to remove solvent. The residue was purified by column chromatography (SiO₂, Petroleum ether: Ethyl acetate=1/0 to 10/1) to give the title compound (9 g, 87% yield) as a white solid. ¹H NMR (400 MHz, CDCl₃-d) δ=8.54-8.36 (m, 1H), 8.14-7.93 (m, 2H), 4.00-3.94 (m, 3H), 1.64-1.61 (m, 9H).

### Step 3—7-Chloro-5-(methoxycarbonyl)benzo[b]thiophene-2-carboxylic acid

To a solution of 02-tert-butyl 05-methyl 7-chlorobenzothiophene-2,5-dicarboxylate (9 g, 30 mmol) in DCM (30 mL) was added TFA (23.03 g, 201.9 mmol, 15.00 mL). The mixture was then stirred at 20° C. for 2 hrs. On completion, the mixture was filtered to give the title compound (7 g, TFA) as a white solid. ¹H NMR (400 MHz, DMSO-d₆) δ=8.55 (d, J=1.4 Hz, 1H), 8.29 (s, 1H), 7.96 (d, J=1.4 Hz, 1H), 3.90 (s, 3H),

### Step 4—Methyl 7-chlorobenzo[b]thiophene-5-carboxylate

A mixture of 7-chloro-5-methoxycarbonyl-benzothiophene-2-carboxylic acid (4 g, 10 mmol, TFA) AgOAc (2.08 g, 12.5 mmol, 639 μL), and K₂CO₃ (1.72 g, 12.5 mmol) in NMP (50 mL) was degassed and purged with N₂ three times at 20° C. Then the mixture was stirred at 120° C. for 15 hrs under N₂ atmosphere. On completion, the reaction mixture was diluted with water (200 mL) and extracted with ethyl acetate (3×100 mL). The combined organic layers were washed with brine (3×100 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 20/1) to give the title compound (2 g, 85% yield) as a white solid. ¹H NMR (400 MHz, CDCl₃-d) δ=8.44 (d, J=1.1 Hz, 1H), 8.01 (d, J=0.8 Hz, 1H), 7.58 (d, J=5.4 Hz, 1H), 7.47 (d, J=5.5 Hz, 1H), 3.98-3.97 (m, 3H).

### Step 5—Methyl 2-bromo-7-chlorobenzo[b]thiophene-5-carboxylate

To a solution of methyl 7-chlorobenzothiophene-5-carboxylate (1.8 g, 7.9 mmol) in THF (50 mL) was added dropwise LDA (2 M, 4.76 mL) at −78° C. After addition, the mixture was stirred at this temperature for 1 hr, and then Br₂ (1.90 g, 11.9 mmol, 614 μL) was added dropwise at −78° C. The resulting mixture was stirred at −78-20° C. for 1 hr. On completion, the reaction mixture was quenched by addition NH₄Cl(aq) (50 mL) at 0° C. and extracted with EA (50 mL×3). The combined organic layers were washed with brine (50 mL×2), dried over Na₂SO₄, filtered and concentrated under reduced pressure to give a residue. The residue was purified by column chromatography (SiO₂, Petroleum ether:Ethyl acetate=1/0 to 30/1) to give the title compound (330 mg, 14% yield) as an off-white solid. ¹H NMR (400 MHz, CDCl₃-d) δ=8.30 (d, J=1.0 Hz, 1H), 8.04-7.95 (m, 1H), 7.50-7.42 (m, 1H), 3.97 (s, 3H).

### Ethyl 2-(1-(3-(1H-1,2,3-triazol-1-yl)propanoyl)-1,2,5,6-tetrahydropyridin-3-yl)-7-chlorobenzo[b]thiophene-5-carboxylic acid (Intermediate UC)

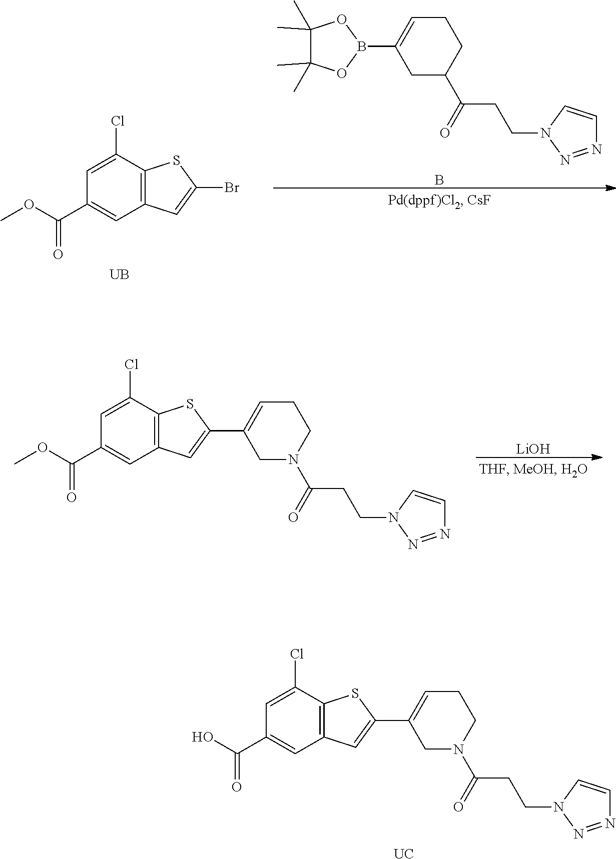

### Step 1—Methyl 2-(1-(3-(1H-1,2,3-triazol-1-yl)propanoyl)-1,2,5,6-tetrahydropyridin-3-yl)-7-chlorobenzo[b]thiophene-5-carboxylate

A mixture of methyl 2-bromo-7-chloro-benzothiophene-5-carboxylate (300 mg, 982 μmol, Intermediate UB), 1-[5-(4,4,5,5-tetramethyl-1,3,2-dioxaborolan-2-yl)-3,6-dihydro-2H-pyridin-1-yl]-3-(triazol-1-yl)propan-1-one (326 mg, 982 μmol, Intermediate B), Pd(dppf)₂Cl₂ (71.8 mg, 98.2 μmol), and CsF (447 mg, 2.95 mmol) in dioxane (5 mL) and H₂O (1 mL) was degassed and purged with N₂ three times at 20° C. Then the mixture was stirred at 80° C. for 1 hr under N₂ atmosphere. On completion, the reaction mixture was concentrated under reduced pressure to remove solvent. The residue was purified by column chromatography (SiO₂, Petroleum ether:Ethyl acetate=20/1 to 0/1) to give the title compound (350 mg, 66% yield) as a yellow solid. LC-MS (ESI⁺) m/z 431.0 (M+H)⁺; ¹H NMR (400 MHz, CHLOROFORM-d) δ=8.41-8.34 (m, 1H), 8.10-8.02 (m, 1H), 7.85-7.81 (m, 1H), 7.78-7.74 (m, 1H), 7.36 (br s, 1H), 6.60-6.48 (m, 1H), 4.91-4.87 (m, 2H), 4.61-4.38 (m, 2H), 4.05 (s, 3H), 3.88-3.62 (m, 2H), 3.25-3.14 (m, 2H), 2.53-2.43 (m, 2H).

### Step 2—Ethyl 2-(1-(3-(1H-1,2,3-triazol-1-yl)propanoyl)-1,2,5,6-tetrahydropyridin-3-yl)-7-chlorobenzo[b]thiophene-5-carboxylic acid

To a solution of methyl 7-chloro-2-[1-[3-(triazol-1-yl)propanoyl]-3,6-dihydro-2H-pyridin-5-yl]benzothiophene-5-carboxylate (280 mg, 650 μmol) in THF (4 mL) and MeOH (1 mL) and H₂O (1 mL) was added LiOH (62.3 mg, 2.60 mmol). Then the mixture was stirred at 40° C. for 1 hr. On completion, the reaction mixture was concentrated under reduced pressure to remove solvent. Then HCl (1N) was added until pH=4, then diluted with water (10 mL) and extracted with ethyl acetate (3×20 mL). The combined organic layers were dried over anhydrous sodium sulfate, filtered and the filtrate was concentrated in vacuo to give the title compound (200 mg, 74% yield) as a yellow solid. LC-MS (ESI⁺) m/z 416.9 (M+H)⁺.

### 1-(3-(7-Chloro-5-(4-(5-fluoro-3-methoxypyridin-2-yl)piperazine-1-carbonyl)benzo[b]thiophen-2-yl)-5,6-dihydropyridin-1(2H)-yl)-3-(1H-1,2,3-triazol-1-yl)propan-1-one (Intermediate UD)

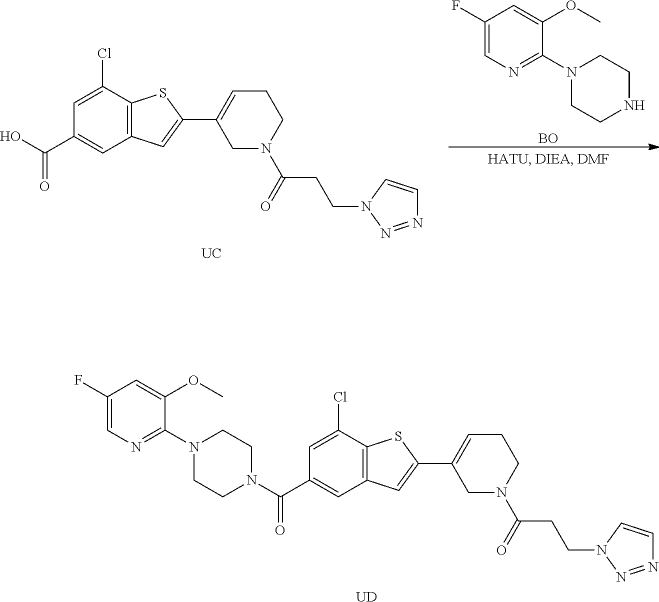 [SMILES: O=C(O)c1cc(Cl)c2sc(C3=CCCN(C(=O)CCn4ccnn4)C3)cc2c1 ; COc1cc(F)cnc1N1CCNCC1 ; "BO" ; "UC" ; COc1cc(F)cnc1N1CCN(C(=O)c2cc(Cl)c3sc(C4=CCCN(C(=O)CCn5ccnn5)C4)cc3c2)CC1 ; "UD"]

To a solution of 7-chloro-2-[1-[3-(triazol-1-yl)propanoyl]-3,6-dihydro-2H-pyridin-5-yl]benzothiophene-5-carboxylic acid (200 mg, 480 μmol, Intermediate UC) in DMF (2 mL) was added dropwise HATU (219 mg, 576 μmol), HOBt (97.2 mg, 720 μmol) and DIEA (310 mg, 2.40 mmol, 418 μL) at 25° C. After addition, the mixture was stirred at 0° C. for 10 mins, and then 1-(5-fluoro-3-methoxy-2-pyridyl)piperazine (119 mg, 480 μmol, HCl, Intermediate BO) was added dropwise at 0° C. The resulting mixture was stirred at 25° C. for 20 mins. On completion, the crude product was triturated with water at 25° C. for 10 mins, then the mixture was filtered and the filter cake was dried in vacuo to give the title compound (220 mg, 75% yield as a brown solid. LC-MS (ESI⁺) m/z 610.1 (M+H)⁺.

### Tert-butyl 5-(1-(cyclopropylmethyl)-5-(4-(5-fluoro-3-methoxypyridin-2-yl)piperazine-1-carbonyl)-7-(4,4,5,5-tetramethyl-1,3,2-dioxaborolan-2-yl)-1H-indol-2-yl)-3,6-dihydropyridine-1(2H)-carboxylate (Intermediate UE)

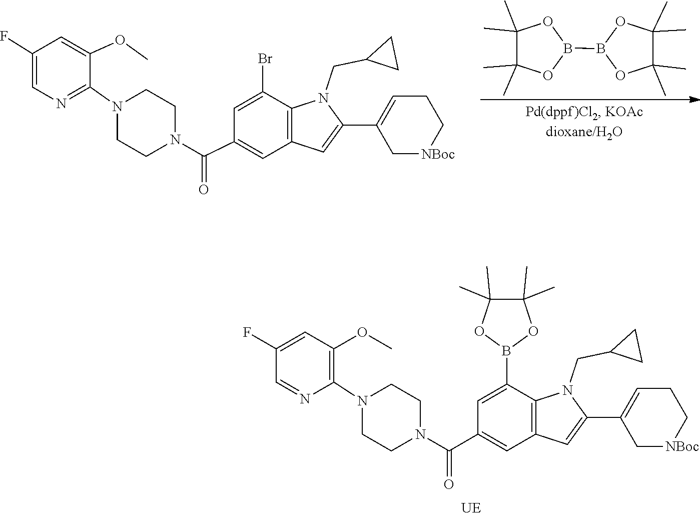

A solution of tert-butyl 5-[7-bromo-1-(cyclopropylmethyl)-5-[4-(5-fluoro-3-methoxy-2-pyridyl) piperazine-1-carbonyl]indol-2-yl]-3,6-dihydro-2H-pyridine-1-carboxylate (1.50 g, 2.24 mmol, synthesized via Step 1 of Intermediate OS), 4,4,5,5-tetramethyl-2-(4,4,5,5-tetramethyl-1,3,2-dioxaborolan-2-yl)-1,3,2-dioxaborolane (2.85 g, 11.2 mmol), Pd(dppf)Cl₂ (164 mg, 224 μmol) and KOAc (660 mg, 6.73 mmol) in dioxane (15 mL) was stirred at 80° C. for 2 hrs under N₂ atmosphere. On completion, the reaction mixture was partitioned between H₂O (100 mL) and EA (100 mL). The organic phase was separated, washed with EA (100 mL×3). The combined organic layers were washed with brine (100 mL), dried over anhydrous sodium sulfate, filtered and the filtrate was concentrated in vacuo to give the crude product. The crude product was purified by silica gel chromatography (SiO₂, PE:EA=10:1 to 5:1) to give the title compound (750 mg, 44% yield) as a yellow solid. LC-MS (ESI⁺) m/z 716.2 (M+H)⁺.

### (1—(Cyclopropylmethyl)-7-(2-ethyl-6-methylpyridin-3-yl)-2-(1,2,5,6-tetrahydropyridin-3-yl)-1H-indol-5-yl)(4-(5-fluoro-3-methoxypyridin-2-yl)piperazin-1-yl)methanone (Intermediate UF)

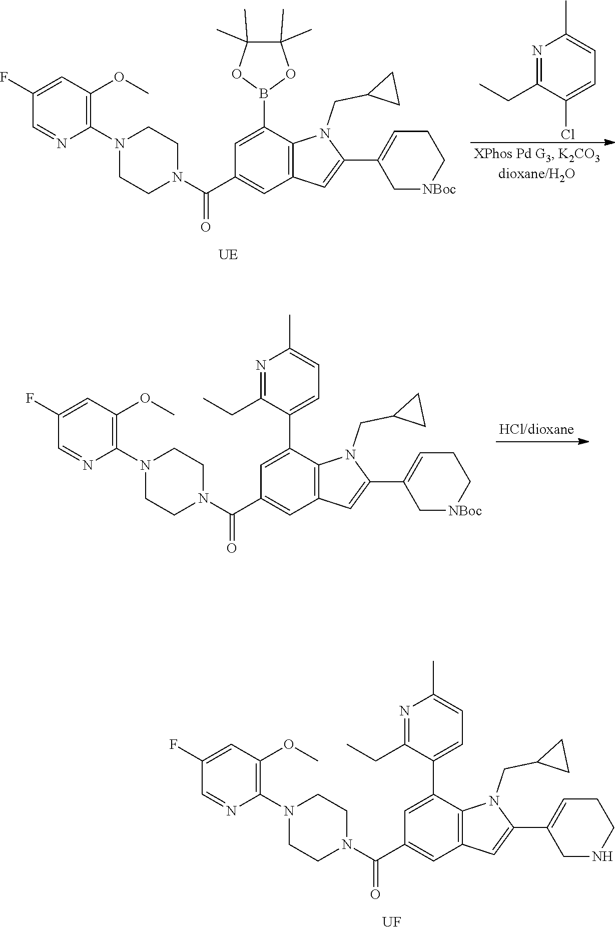

### Step 1—Tert-butyl 3-(1-(cyclopropylmethyl)-7-(2-ethyl-6-methylpyridin-3-yl)-5-(4-(5-fluoro-3-methoxypyridin-2-yl)piperazine-1-carbonyl)-1H-indol-2-yl)-5,6-dihydropyridine-1(2H)-carboxylate

To a solution of tert-butyl 5-[1-(cyclopropylmethyl)-5-[4-(5-fluoro-3-methoxy-2-pyridyl)piperazine-1-carbonyl]-7-(4,4,5,5-tetramethyl-1,3,2-dioxaborolan-2-yl)indol-2-yl]-3,6-dihydro-2H-pyridine-1-carboxylate (300 mg, 419 μmol, Intermediate UE), 3-chloro-2-ethyl-6-methyl-pyridine (97.8 mg, 628 μmol) and XPhos Pd G3 (35.4 mg, 41.9 μmol) in dioxane (3 mL) and H₂O (0.6 mL) was added K₂CO₃ (173 mg, 1.26 mmol). The mixture was then stirred at 80° C. for 2 hrs under N₂ atmosphere. On completion, the reaction mixture was partitioned between H₂O (10 mL) and EA (20 mL). The organic phase was separated, washed with EA (20 mL×3). The combined organic layers were washed with brine (20 mL), dried over anhydrous sodium sulfate, filtered and the filtrate was concentrated in vacuo to give the crude. The crude product was purified by reversed-phase HPLC (0.1% FA condition) to give the title compound (210 mg, 64% yield) as a yellow solid. LC-MS (ESI⁺) m/z 709.4 (M+H)⁺.

### Step 2—(1—(Cyclopropylmethyl)-7-(2-ethyl-6-methylpyridin-3-yl)-2-(1,2,5,6-tetrahydropyridin-3-yl)-1H-indol-5-yl)(4-(5-fluoro-3-methoxypyridin-2-yl)piperazin-1-yl)methanone

A solution of tert-butyl 5-[1-(cyclopropylmethyl)-7-(2-ethyl-6-methyl-3-pyridyl)-5-[4-(5-fluoro-3-methoxy-2-pyridyl)piperazine-1-carbonyl]indol-2-yl]-3,6-dihydro-2H-pyridine-1-carboxylate (210 mg, 296 μmol) in HCl/dioxane (2 M, 2.10 mL) was stirred at 20° C. for 2 hrs. On completion, the reaction mixture was filtered and concentrated under reduced pressure to give the title compound (180 mg, 94% yield, HCl) as a yellow solid. LC-MS (ESI⁺) m/z 609.5 (M+H)⁺.

### (1—(Cyclopropylmethyl)-7-(2-ethyl-6-methylpyridin-3-yl)-3-fluoro-2-(1,2,5,6-tetrahydropyridin-3-yl)-1H-indol-5-yl)(4-(5-fluoro-3-methoxypyridin-2-yl)piperazin-1-yl)methanone (Intermediate UG)

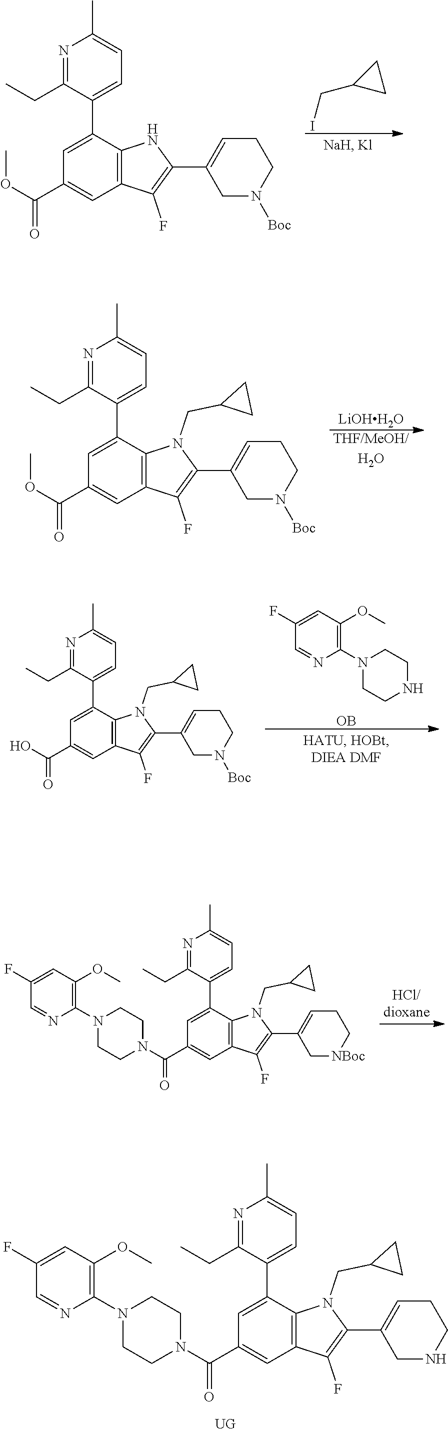

### Step 1—Methyl 2-(1-(tert-butoxycarbonyl)-1,2,5,6-tetrahydropyridin-3-yl)-1-(cyclopropylmethyl)-7-(2-ethyl-6-methylpyridin-3-yl)-3-fluoro-1H-indole-5-carboxylate

To a solution of methyl 2-(1-tert-butoxycarbonyl-3,6-dihydro-2H-pyridin-5-yl)-7-(2-ethyl-6-methyl-3-pyridyl)-3-fluoro-1H-indole-5-carboxylate (170 mg, 227.32 μmol, synthesized via Step 1 of Intermediate SX) in DMF (1 mL) was added NaH (13.64 mg, 340.99 μmol) and the mixture was stirred at 0° C. for 0.5 hr. Then added iodomethylcyclopropane (53.79 mg, 295.52 μmol, CAS #33574-02-6) and the mixture was stirred at 0-25° C. for 11.5 hr. Upon completion, water (10 mL) was added to the mixture and ethyl acetate (10×3 mL) was used for extraction, then the organic phase drying was concentrated under reduced pressure. The crude product was purified by reversed-phase HPLC(0.1% FA condition) to give the title compound (85 mg, 62% yield) as a white solid. LC-MS (ESI⁺) m/z 548.9 (M+H)⁺.

### Step 2—2-(1-(Tert-butoxycarbonyl)-1,2,5,6-tetrahydropyridin-3-yl)-1-(cyclopropylmethyl)-7-(2-ethyl-6-methylpyridin-3-yl)-3-fluoro-1H-indole-5-carboxylic acid

To a solution of methyl 2-(1-tert-butoxycarbonyl-3,6-dihydro-2H-pyridin-5-yl)-1-(cyclopropylmethyl)-7-(2-ethyl-6-methyl-3-pyridyl)-3-fluoro-indole-5-carboxylate (85 mg, 155.21 μmol) in THF (0.8 mL) and MeOH (0.2 mL) was added LiOH·H₂O (19.54 mg, 465.62 μmol) and H₂O (0.2 mL) and the mixture was stirred at 40° C. for 12 hr. Upon completion, the mixture was concentrated under reduced pressure to afford the title compound (85 mg) as a yellow solid. LC-MS (ESI⁺) m/z 534.9 (M+H)⁺.

### Step 3—Tert-butyl 3-(1-(cyclopropylmethyl)-7-(2-ethyl-6-methylpyridin-3-yl)-3-fluoro-5-(4-(5-fluoro-3-methoxypyridin-2-yl)piperazine-1-carbonyl)-1H-indol-2-yl)-5,6-dihydropyridine-1(2H)-carboxylate

To a solution of 2-(1-tert-butoxycarbonyl-3,6-dihydro-2H-pyridin-5-yl)-1-(cyclopropylmethyl)-7-(2-ethyl-6-methyl-3-pyridyl)-3-fluoro-indole-5-carboxylic acid (100 mg, 187.39 mol) in DMF (1 mL) was added HATU (106.88 mg, 281.09 μmol), HOBt (50.64 mg, 374.79 μmol) and DIEA (121.10 mg, 936.97 μmol) and the mixture was stirred at 25° C. for 6 min. Then 1-(5-fluoro-3-methoxy-2-pyridyl)piperazine (47.50 mg, 224.9 μmol, Intermediate BO) was added and the mixture was stirred at 25° C. for 24 min. On completion, the mixture was filtered. The crude product was purified by reversed-phase HPLC(0.1% FA condition) to give the title compound (60 mg, 41% yield) as a white solid. LC-MS (ESI⁺) m/z 727.5 (M+H)⁺.

### Step 4—(1-(cyclopropylmethyl)-7-(2-ethyl-6-methylpyridin-3-yl)-3-fluoro-2-(1,2,5,6-tetrahydropyridin-3-yl)-1H-indol-5-yl)(4-(5-fluoro-3-methoxypyridin-2-yl)piperazin-1-yl)methanone

A solution of tert-butyl 5-[1-(cyclopropylmethyl)-7-(2-ethyl-6-methyl-3-pyridyl)-3-fluoro-5-[4-(5-fluoro-3-methoxy-2-pyridyl)piperazine-1-carbonyl]indol-2-yl]-3,6-dihydro-2H-pyridine-1-carboxylate (100 mg, 137.58 μmol) in HCl/dioxane (2 M, 2 mL) was stirred at 25° C. for 10 min. On completion, the mixture was concentrated under reduced pressure to obtain the title compound (100 mg) as a yellow solid. LC-MS (ESI⁺) m/z 627.3 (M+H)⁺.

### (1—(Cyclopropylmethyl)-7-(2-ethyl-6-methylpyridin-3-yl)-3-fluoro-2-(1,2,5,6-tetrahydropyridin-3-yl)-1H-indol-5-yl)(1-methylpyrrolo[3,4-c]pyrazol-5(1H,4H,6H)-yl)methanone (Intermediate UH)

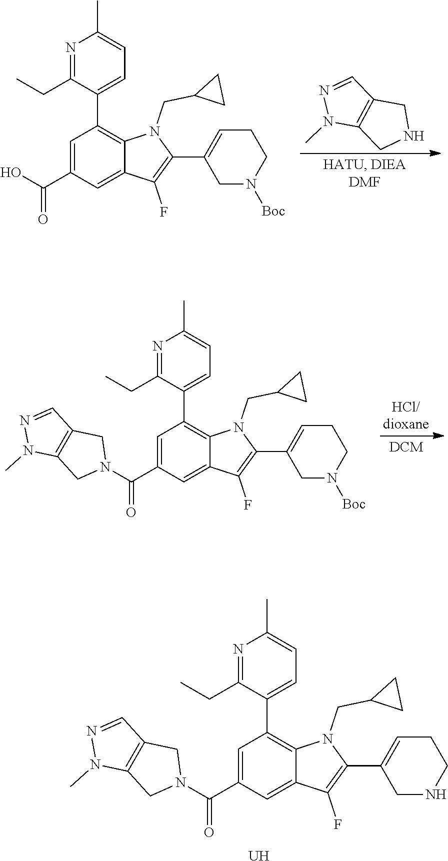

### Step 1—Tert-butyl 3-(1-(cyclopropylmethyl)-7-(2-ethyl-6-methylpyridin-3-yl)-3-fluoro-5-(1-methyl-1,4,5,6-tetrahydropyrrolo[3,4-c]pyrazole-5-carbonyl)-1H-indol-2-yl)-5,6-dihydropyridine-1(2H)-carboxylate

A mixture of 2-(1-(tert-butoxycarbonyl)-1,2,5,6-tetrahydropyridin-3-yl)-1-(cyclopropylmethyl)-7-(2-ethyl-6-methylpyridin-3-yl)-3-fluoro-1H-indole-5-carboxylic acid (120 mg, 224 mol, synthesized via Steps 1-2 of Intermediate UG), 1-methyl-1,4,5,6-tetrahydropyrrolo[3,4-c]pyrazole (53.8 mg, 337 μmol, HCl, CAS #762233-62-5), HATU (102 mg, 269 μmol), and DIEA (145 mg, 1.12 mmol) in DMF (1 mL) was stirred at 25° C. for 2 hrs. On completion, the crude residue was purified by reversed-phase HPLC (0.8 g/L ammonium bicarbonate) to give the title compound (130 mg) as a yellow solid. LC-MS (ESI⁺) m/z 639.4 (M+H)⁺.

### Step 2—(1—(Cyclopropylmethyl)-7-(2-ethyl-6-methylpyridin-3-yl)-3-fluoro-2-(1,2,5,6-tetrahydropyridin-3-yl)-1H-indol-5-yl)(1-methylpyrrolo[3,4-c]pyrazol-5(1H,4H,6H)-yl)methanone

A mixture of tert-butyl 3-(1-(cyclopropylmethyl)-7-(2-ethyl-6-methylpyridin-3-yl)-3-fluoro-5-(1-methyl-1,4,5,6-tetrahydropyrrolo[3,4-c]pyrazole-5-carbonyl)-1H-indol-2-yl)-5,6-dihydropyridine-1(2H)-carboxylate (130 mg, 203 μmol) in DCM (1 mL) and HCl/dioxane (1 mL) was stirred at 25° C. for 2 hrs. On completion, the mixture was concentrated in vacuo give the title compound (120 mg) as a yellow solid.

### (S)-methyl 7-bromo-2-(1-(tert-butoxycarbonyl)piperidin-3-yl)-3-fluoro-1H-indole-5-carboxylate (Intermediate UI) & (R)-methyl 7-bromo-2-(1-(tert-butoxycarbonyl)piperidin-3-yl)-3-fluoro-1H-indole-5-carboxylate (Intermediate UJ)

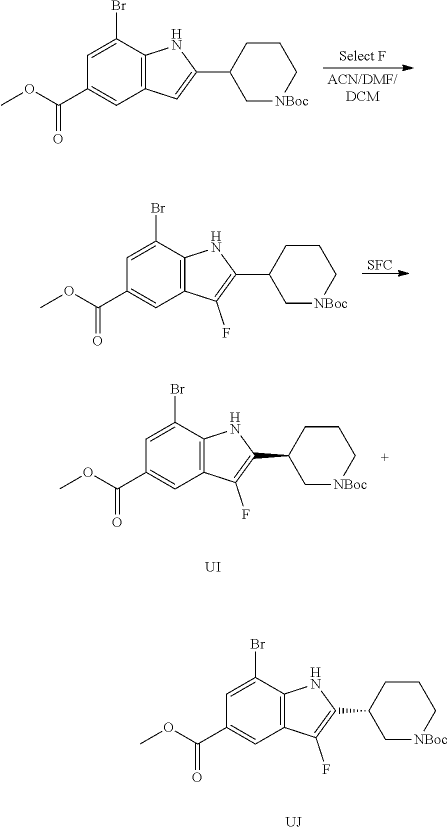

### Step 1—Methyl 7-bromo-2-(1-(tert-butoxycarbonyl)piperidin-3-yl)-3-fluoro-1H-indole-5-carboxylate

To a solution of methyl 7-bromo-2-(1-(tert-butoxycarbonyl)piperidin-3-yl)-1H-indole-5-carboxylate (40 g, 91.5 mmol, synthesized via Steps 1-3 of Intermediate NT) in ACN (400 mL) and DCM (200 mL) was added 1-(chloromethyl)-4-fluoro-1,4-diazoniabicyclo[2.2.2]octane;ditetrafluoroborate (32.4 g, 91.5 mmol) in DMF (160 mL). Then the mixture was stirred at 0° C. for 10 mins. On completion, the reaction mixture was concentrated in vacuo and diluted with water (1000 mL) and extracted with EA (3×200 mL). The combined organic layers were washed with brine (600 mL) and dried over anhydrous sodium sulfate, filtered and the filtrate was concentrated in vacuo to give the crude residue. The residue was purified by reversed-phase HPLC (0.1% FA condition) to give the title compound (14 g, 33% yield) as a yellow solid. LC-MS (ESI⁺) m/z 399.0 (M−55)⁺.

### Step 2—(S)-methyl 7-bromo-2-(1-(tert-butoxycarbonyl)piperidin-3-yl)-3-fluoro-1H-indole-5-carboxylate & (R)-methyl 7-bromo-2-(1-(tert-butoxycarbonyl)piperidin-3-yl)-3-fluoro-1H-indole-5-carboxylate

Methyl 7-bromo-2-(1-(tert-butoxycarbonyl)piperidin-3-yl)-3-fluoro-1H-indole-5-carboxylate (14 g, 30.8 mmol) was purified by SFC (column: DAICEL CHIRALPAK IG(250 mm*50 mm,10 um);mobile phase: [CO₂-MeOH(0.1% NH₃H₂O)]; gradient:30%-% B over 4.5 min) to give the first peak as (S)-methyl 7-bromo-2-(1-(tert-butoxycarbonyl)piperidin-3-yl)-3-fluoro-1H-indole-5-carboxylate (7 g, 48% yield) as a white solid and the second peak as (R)-methyl 7-bromo-2-(1-(tert-butoxycarbonyl)piperidin-3-yl)-3-fluoro-1H-indole-5-carboxylate (6.2 g, 43% yield) as a white solid. LC-MS (ESI⁺) m/z 398.9 (M−55)⁺. The absolute stereochemistry of the enantiomers was assigned arbitrarily.

### (S)-methyl 7-bromo-2-(1-(tert-butoxycarbonyl)piperidin-3-yl)-1-(cyclopropylmethyl)-3-fluoro-1H-indole-5-carboxylate (Intermediate UK)

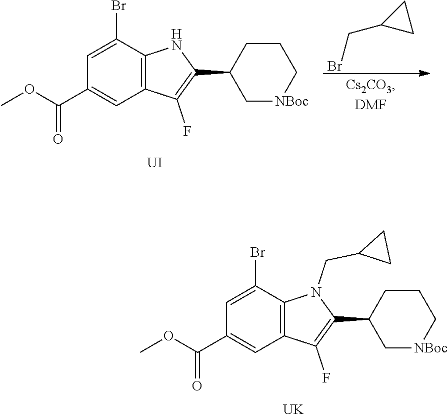

A mixture of (S)-methyl 7-bromo-2-(1-(tert-butoxycarbonyl)piperidin-3-yl)-3-fluoro-1H-indole-5-carboxylate (5.7 g, 13 mmol, Intermediate UI) in DMF (60 mL) was added Cs₂CO₃ (12.2 g, 37.6 mmol) and (bromomethyl)cyclopropane (3.38 g, 25.0 mmol). The mixture was stirred at 25° C. for 12 hrs. On completion, the reaction mixture was diluted with water (250 mL) and extracted with EA (3×40 mL). The combined organic layers were washed with brine (200 mL) and dried over anhydrous sodium sulfate, filtered and the filtrate was concentrated in vacuo to give the crude residue. The residue was purified by column chromatography (SiO₂, Petroleum ether:Ethyl acetate=0/1 to 50/1) to give the title compound (5.3 g, 81% yield) as a yellow solid. LC-MS (ESI⁺) m/z 453.9 (M−55)⁺; ¹H NMR (400 MHz, DMSO-d6) δ=8.09 (s, 1H), 7.91 (s, 1H), 4.79-4.40 (m, 2H), 4.17-3.97 (m, 3H), 3.87 (s, 3H), 3.17-3.07 (m, 1H), 2.08-1.96 (m, 2H), 1.95-1.83 (m, 1H), 1.73 (d, J=12.0 Hz, 1H), 1.60-1.50 (m, 1H), 1.40 (s, 9H), 1.18 (d, J=6.8 Hz, 1H), 0.52-0.36 (m, 4H).

### (S)-methyl 2-(1-(tert-butoxycarbonyl)piperidin-3-yl)-1-(cyclopropylmethyl)-3-fluoro-7-(4,4,5,5-tetramethyl-1,3,2-dioxaborolan-2-yl)-1H-indole-5-carboxylate (Intermediate UL)

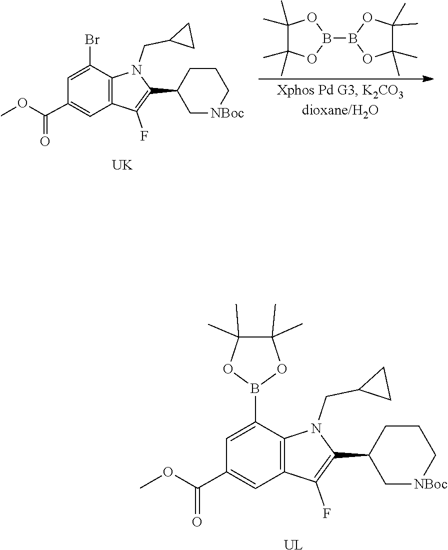

A mixture of (S)-methyl 7-bromo-2-(1-(tert-butoxycarbonyl)piperidin-3-yl)-1-(cyclopropylmethyl)-3-fluoro-1H-indole-5-carboxylate (4 g, 8 mmol, Intermediate UK) and 4,4,5,5-tetramethyl-2-(4,4,5,5-tetramethyl-1,3,2-dioxaborolan-2-yl)-1,3,2-dioxaborolane (7.98 g, 31.4 mmol) in dioxane (40 mL) and H₂O (4 mL) was added XPhos Pd G3 (665 mg, 785 μmol) and K₂CO₃ (3.26 g, 23.6 mmol). The mixture was then stirred at 80° C. for 1 hr. On completion, the reaction mixture was filtrated and concentrated in vacuo to give the crude residue. The crude residue was purified by column chromatography (SiO2, Petroleum ether:Ethyl acetate=100/1 to 10/1) to give the title compound (3.6 g, 61% yield) as a yellow solid. LC-MS (ESI+) m/z 557.1 (M+H)⁺.

### (S)-2-(1-(tert-butoxycarbonyl)piperidin-3-yl)-1-(cyclopropylmethyl)-7-(2-ethyl-6-methylpyridin-3-yl)-3-fluoro-1H-indole-5-carboxylic acid (Intermediate UM)

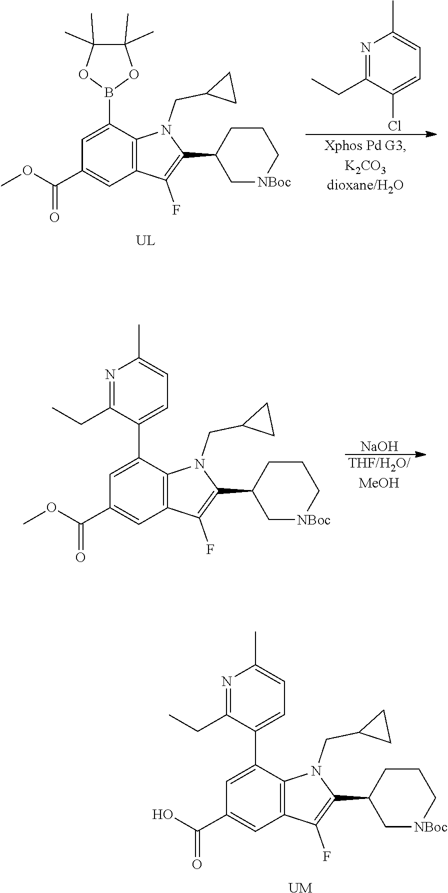

### Step 1—(S)-methyl 2-(1-(tert-butoxycarbonyl)piperidin-3-yl)-1-(cyclopropylmethyl)-7-(2-ethyl-6-methylpyridin-3-yl)-3-fluoro-1H-indole-5-carboxylate

A mixture of methyl 2-[(3S)-1-tert-butoxycarbonyl-3-piperidyl]-1-(cyclopropylmethyl)-3-fluoro-7-(4,4,5,5-tetramethyl-1,3,2-dioxaborolan-2-yl)indole-5-carboxylate (2.3 g, 4.1 mmol, Intermediate UL), 3-chloro-2-ethyl-6-methyl-pyridine (643 mg, 4.13 mmol), XPhos Pd G3 (349 mg, 413 μmol), and K₃PO₄ (2.63 g, 12.4 mmol) in H₂O (5 mL) and dioxane (20 mL) was degassed and purged with N₂ three times. Then the mixture was stirred at 80° C. for 1 hr under N₂ atmosphere. On completion, the reaction mixture was filtered and concentrated under reduced pressure to give a residue. The crude product was purified by reversed-phase HPLC(0.1% FA condition) to afford the title compound (1.2 g, 40% yield) as a white solid. LC-MS (ESI+) m/z 550.4 (M+H)⁺.

### Step 2—(S)-2-(1-(tert-butoxycarbonyl)piperidin-3-yl)-1-(cyclopropylmethyl)-7-(2-ethyl-6-methylpyridin-3-yl)-3-fluoro-1H-indole-5-carboxylic acid

To a solution of methyl 2-[(3S)-1-tert-butoxycarbonyl-3-piperidyl]-1-(cyclopropylmethyl)-7-(2-ethyl-6-methyl-3-pyridyl)-3-fluoro-indole-5-carboxylate (1.2 g, 2.2 mmol) in THF (8 mL), MeOH (2 mL) and H₂O (2 mL) was added NaOH (611 mg, 15.2 mmol). The mixture was then stirred at 25° C. for 12 hrs. On completion, the reaction mixture was quenched with 2 N HCl (10 mL) at 25° C., and then diluted with H₂O (10 mL) and extracted with EA (8 mL×3). The combined organic layers were washed with aqueous NaCl (10 mL×2), dried over Na₂SO₄, filtered and concentrated under reduced pressure to give a residue. The crude product was triturated with PE (20 mL) at 25° C. for 30 mins to afford the title compound (1.2 g, 82% yield) as a yellow solid. LC-MS (ESI+) m/z 536.4 (M+H)⁺.

### (S)-(1-(cyclopropylmethyl)-7-(2-ethyl-6-methylpyridin-3-yl)-3-fluoro-2-(piperidin-3-yl)-1H-indol-5-yl)(1-methylpyrrolo[3,4-c]pyrazol-5(1H,4H,6H)-yl)methanone (Intermediate UN)

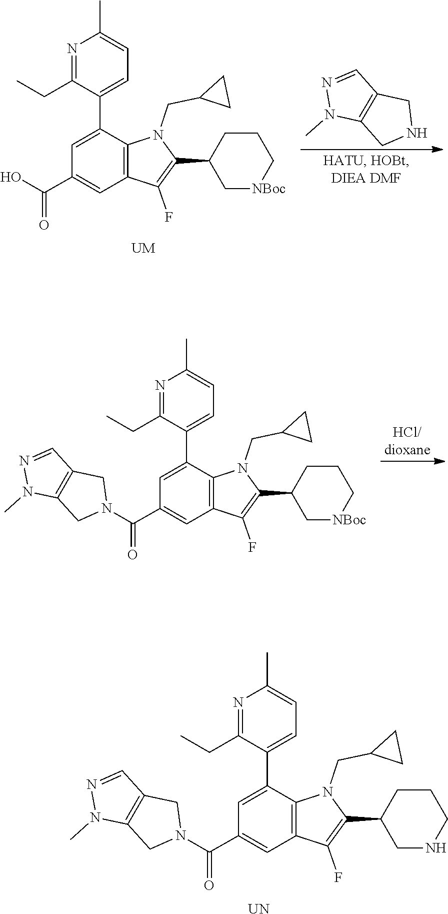

### Step 1—(S)-tert-butyl 3-(1-(cyclopropylmethyl)-7-(2-ethyl-6-methylpyridin-3-yl)-3-fluoro-5-(1-methyl-1,4,5,6-tetrahydropyrrolo[3,4-c]pyrazole-5-carbonyl)-1H-indol-2-yl)piperidine-1-carboxylate

To a solution of 2-[(3S)-1-tert-butoxycarbonyl-3-piperidyl]-1-(cyclopropylmethyl)-7-(2-ethyl-6-methyl-3-pyridyl)-3-fluoro-indole-5-carboxylic acid (600 mg, 1.12 mmol, Intermediate UM) in DMF (10 mL) was added HOBt (302 mg, 2.24 mmol), HATU (638 mg, 1.68 mmol), DIEA (723 mg, 5.60 mmol) and 1-methyl-5,6-dihydro-4H-pyrrolo[3,4-c]pyrazole hydrochloride (178 mg, 1.12 mmol). The mixture was then stirred at 25° C. for 10 mins. On completion, the reaction mixture was diluted with H₂O (30 mL) and extracted with EA (10 mL×3). The combined organic layers were washed with aqueous NaCl (10 mL×2), dried over Na₂SO₄, filtered and concentrated under reduced pressure to give a residue. The crude product was triturated with PE (20 mL) at 25° C. for 30 minutes to afford the title compound (800 mg, 93% yield) as a yellow gum. LC-MS (ESI+) m/z 641.5 (M+H)⁺.

### Step 2—(S)-(1-(cyclopropylmethyl)-7-(2-ethyl-6-methylpyridin-3-yl)-3-fluoro-2-(piperidin-3-yl)-1H-indol-5-yl)(1-methylpyrrolo[3,4-c]pyrazol-5(1H,4H,6H)-yl)methanone

A solution of tert-butyl (3S)-3-[1-(cyclopropylmethyl)-7-(2-ethyl-6-methyl-3-pyridyl)-3-fluoro-5-(1-methyl-4,6-dihydropyrrolo[3,4-c]pyrazole-5-carbonyl)indol-2-yl]piperidine-1-carboxylate (800 mg, 1.25 mmol) in HCl/dioxane (2 M, 8 mL) was stirred at 25° C. for 1 hr. On completion, the reaction mixture was filtered and concentrated under reduced pressure to give a residue. The crude product was triturated with EA at 25° C. for 10 mins to afford the title compound (800 mg, 88% yield, HCl) as a yellow gum. LC-MS (ESI+) m/z 541.3 (M+H)⁺.

### (R)-methyl 7-bromo-2-(1-(tert-butoxycarbonyl)piperidin-3-yl)-1-(cyclopropylmethyl)-3-fluoro-1H-indole-5-carboxylate (Intermediate UO)

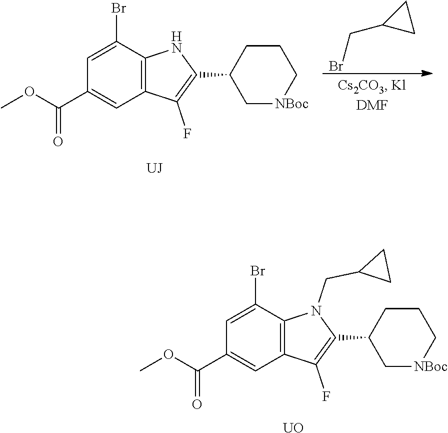

To a solution of (R)-methyl 7-bromo-2-(1-(tert-butoxycarbonyl)piperidin-3-yl)-3-fluoro-1H-indole-5-carboxylate (5 g, 10 mmol, Intermediate UJ), (bromomethyl)cyclopropane (2.97 g, 21.9 mmol) in DMF (50 mL) was added Cs₂CO₃ (10.7 g, 32.9 mmol) and KI (911 mg, 5.49 mmol). The mixture was then stirred at 25° C. for 12 hrs. On completion, the reaction mixture was quenched with water (100 mL) and extracted with ethyl acetate (3×100 mL). The combined organic layers were washed with brine (2×100 mL) and dried over anhydrous sodium sulfate, filtered and the filtrate was concentrated in vacuo to give the title compound (5.6 g) as a yellow solid. LC-MS (ESI⁺) m/z 452.9 (M−55)⁺.

### (R)-methyl 2-(1-(tert-butoxycarbonyl)piperidin-3-yl)-1-(cyclopropylmethyl)-3-fluoro-7-(4,4,5,5-tetramethyl-1,3,2-dioxaborolan-2-yl)-1H-indole-5-carboxylate (Intermediate UP)

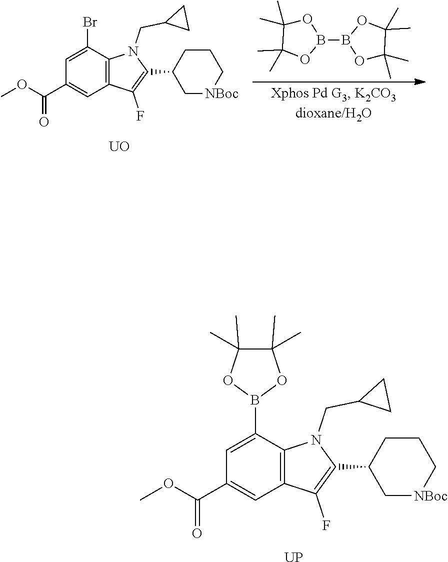

A mixture of (R)-methyl 7-bromo-2-(1-(tert-butoxycarbonyl)piperidin-3-yl)-1-(cyclopropylmethyl)-3-fluoro-1H-indole-5-carboxylate (5.5 g, 11 mmol, Intermediate UO), 4,4,4′,4′,5,5,5′,5′-octamethyl-2,2′-bi(1,3,2-dioxaborolane) (13.71 g, 54.0 mmol), XPhos Pd G3 (914 mg, 1.08 mmol), and K₂CO₃ (4.48 g, 32.4 mmol) in dioxane (100 mL) and H₂O (10 mL) was degassed and purged with N₂ three times. Then the mixture was stirred at 80° C. for 1 hr under N₂ atmosphere. On completion, the reaction mixture was concentrated in vacuo to give the residue. The residue was purified by column chromatography (SiO₂, Petroleum ether/Ethyl acetate=1/0 to 20/1) to give the title compound (5 g, 69% yield) as a yellow solid. LC-MS (ESI⁺) m/z 557.3 (M+H)⁺.

### (R)-2-(1-(tert-butoxycarbonyl)piperidin-3-yl)-1-(cyclopropylmethyl)-7-(2-ethyl-6-methylpyridin-3-yl)-3-fluoro-1H-indole-5-carboxylic acid (Intermediate UQ)

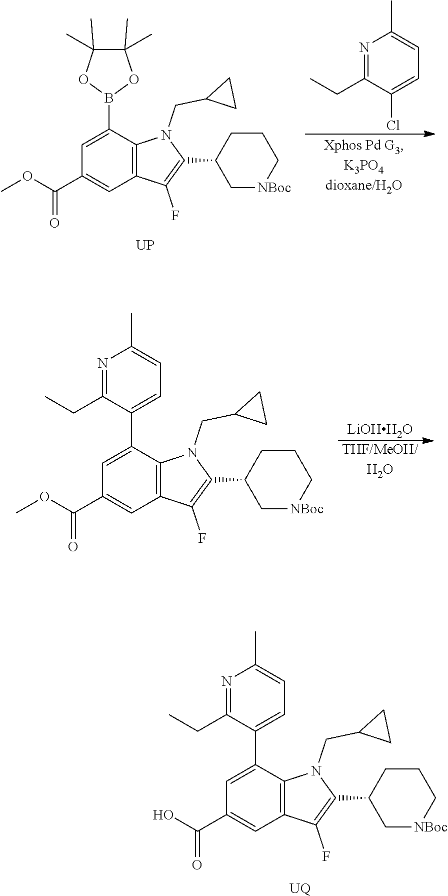

### Step 1—(R)-methyl 2-(1-(tert-butoxycarbonyl)piperidin-3-yl)-1-(cyclopropylmethyl)-7-(2-ethyl-6-methylpyridin-3-yl)-3-fluoro-1H-indole-5-carboxylate

A mixture of (R)-methyl 2-(1-(tert-butoxycarbonyl)piperidin-3-yl)-1-(cyclopropylmethyl)-3-fluoro-7-(4,4,5,5-tetramethyl-1,3,2-dioxaborolan-2-yl)-1H-indole-5-carboxylate (3 g, 5.39 mmol, Intermediate UP), 3-chloro-2-ethyl-6-methylpyridine (839 mg, 5.39 mmol), XPhos Pd G3 (456 mg, 539 μmol), and K₃PO₄ (3.43 g, 16.2 mmol) in dioxane (30 mL) and H₂O (6 mL) was degassed and purged with N₂ three times. Then the mixture was stirred at 80° C. for 1 hr under N₂ atmosphere. On completion, the reaction mixture was concentrated in vacuo to give the residue. The residue was purified by column chromatography (SiO₂, Petroleum ether/Ethyl acetate=1/0 to 5/1) to give the title compound (2.2 g, 53% yield) as a yellow solid. LC-MS (ESI⁺) m/z 550.8 (M+H)⁺.

### Step 2—(R)-2-(1-(tert-butoxycarbonyl)piperidin-3-yl)-1-(cyclopropylmethyl)-7-(2-ethyl-6-methylpyridin-3-yl)-3-fluoro-1H-indole-5-carboxylic acid

To a solution of (R)-methyl 2-(1-(tert-butoxycarbonyl)piperidin-3-yl)-1-(cyclopropylmethyl)-7-(2-ethyl-6-methylpyridin-3-yl)-3-fluoro-1H-indole-5-carboxylate (2.2 g, 4.0 mmol) in THF (20 mL), MeOH (5 mL) and H₂O (5 mL) was added LiOH·H₂O (1.68 g, 40.0 mmol). The mixture was then stirred at 40° C. for 12 hrs. On completion, the reaction mixture was quenched with HCl (2 M) until the pH=2-3, and extracted with ethyl acetate (3×50 mL). The combined organic layers were washed with brine (2×30 mL) and dried over anhydrous sodium sulfate, filtered and the filtrate was concentrated in vacuo to give the title compound (2 g) as a brown solid. LC-MS (ESI⁺) m/z 536.7 (M+H)⁺.

### (R)-(1-(cyclopropylmethyl)-7-(2-ethyl-6-methylpyridin-3-yl)-3-fluoro-2-(piperidin-3-yl)-1H-indol-5-yl)(1-methylpyrrolo[3,4-c]pyrazol-5(1H,4H,6H)-yl)methanone (Intermediate UR)

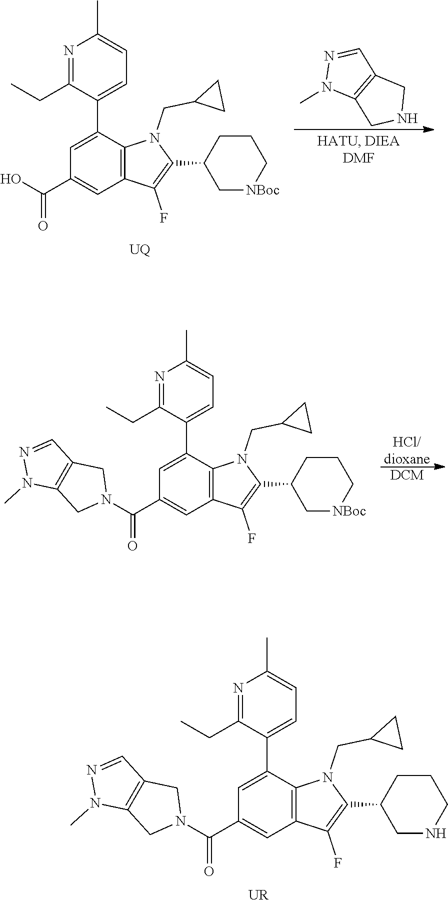

### Step 1—(R)-tert-butyl 3-(1-(cyclopropylmethyl)-7-(2-ethyl-6-methylpyridin-3-yl)-3-fluoro-5-(1-methyl-1,4,5,6-tetrahydropyrrolo[3,4-c]pyrazole-5-carbonyl)-1H-indol-2-yl)piperidine-1-carboxylate

To a solution of (R)-2-(1-(tert-butoxycarbonyl)piperidin-3-yl)-1-(cyclopropylmethyl)-7-(2-ethyl-6-methylpyridin-3-yl)-3-fluoro-1H-indole-5-carboxylic acid (900 mg, 1.68 mmol, Intermediate UQ) in DMF (15 mL) was added HATU (958 mg, 2.52 mmol) and DIEA (1.09 g, 8.40 mmol). After addition, the mixture was stirred at this 25° C. for 10 min, and then 1-methyl-1,4,5,6-tetrahydropyrrolo[3,4-c]pyrazole (402 mg, 2.52 mmol) was added. The resulting mixture was stirred at 40° C. for 20 min. On completion, the reaction mixture was diluted with water (50 mL) and extracted with ethyl acetate (3×50 mL). The combined organic layers were washed with brine (2×30 mL) and dried over anhydrous sodium sulfate, filtered and the filtrate was concentrated in vacuo to give the title compound (1 g) as a yellow solid. LC-MS (ESI⁺) m/z 641.3 (M+H)⁺.

### Step 2—(R)-(1-(cyclopropylmethyl)-7-(2-ethyl-6-methylpyridin-3-yl)-3-fluoro-2-(piperidin-3-yl)-1H-indol-5-yl)(1-methylpyrrolo[3,4-c]pyrazol-5(1H,4H,6H)-yl)methanone

A solution of (R)-tert-butyl 3-(1-(cyclopropylmethyl)-7-(2-ethyl-6-methylpyridin-3-yl)-3-fluoro-5-(1-methyl-1,4,5,6-tetrahydropyrrolo[3,4-c]pyrazole-5-carbonyl)-1H-indol-2-yl)piperidine-1-carboxylate (1 g, 2 mmol) in DCM (5 mL) was added HCl/dioxane (2 M, 5 mL) was stirred at 25° C. for 1 hr. On completion, the reaction mixture was concentrated in vacuo to give the title compound (800 mg) as a yellow solid. LC-MS (ESI⁺) m/z 541.4 (M+H)⁺.

### (R)-2-(1-(tert-butoxycarbonyl)piperidin-3-yl)-1-(cyclopropylmethyl)-7-(3-ethylpyridin-4-yl)-3-fluoro-1H-indole-5-carboxylic acid (Intermediate US)

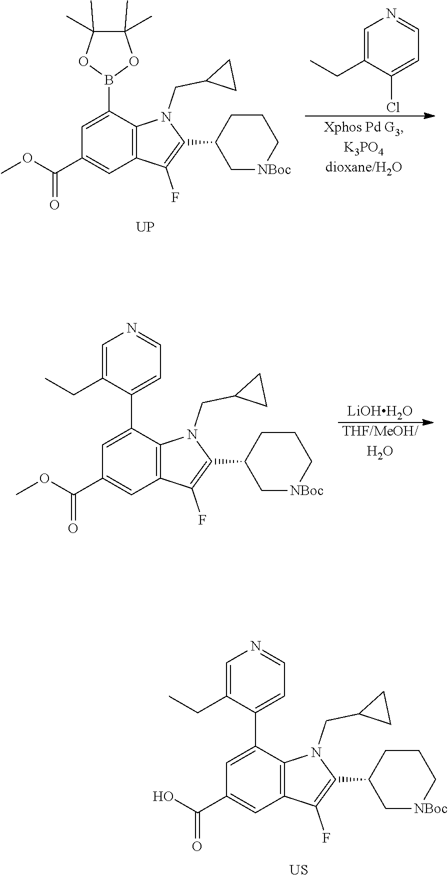

### Step 1—(R)-methyl 2-(1-(tert-butoxycarbonyl)piperidin-3-yl)-1-(cyclopropylmethyl)-7-(3-ethylpyridin-4-yl)-3-fluoro-1H-indole-5-carboxylate

A mixture of (R)-methyl 2-(1-(tert-butoxycarbonyl)piperidin-3-yl)-1-(cyclopropylmethyl)-3-fluoro-7-(4,4,5,5-tetramethyl-1,3,2-dioxaborolan-2-yl)-1H-indole-5-carboxylate (1 g, 1.80 mmol, Intermediate UP), 4-chloro-3-ethylpyridine (254 mg, 1.80 mmol, CAS #860411-22-9), XPhos Pd G3 (152 mg, 179 μmol), and K₃PO₄ (1.14 g, 5.39 mmol) in dioxane (10 mL) and H₂O (2 mL) was degassed and purged with N₂ three times. Then the mixture was stirred at 60° C. for 1 hr under N₂ atmosphere. On completion, the reaction mixture was concentrated in vacuo to give the residue. The residue was purified by column chromatography (SiO₂, Petroleum ether/Ethyl acetate=1/0 to 4/1) to give the title compound (720 mg, 72% yield) as a yellow solid. LC-MS (ESI⁺) m/z 536.7 (M+H)⁺.

### Step 2—(R)-2-(1-(tert-butoxycarbonyl)piperidin-3-yl)-1-(cyclopropylmethyl)-7-(3-ethylpyridin-4-yl)-3-fluoro-1H-indole-5-carboxylic acid

To a solution of (R)-methyl 2-(1-(tert-butoxycarbonyl)piperidin-3-yl)-1-(cyclopropylmethyl)-7-(3-ethylpyridin-4-yl)-3-fluoro-1H-indole-5-carboxylate (720 mg, 1.34 mmol) in THF (8 mL), MeOH (2 mL) and H₂O (2 mL) was added LiOH·H₂O (564 mg, 13.4 mmol). The mixture was then stirred at 40° C. for 12 hrs. On completion, the reaction mixture was quenched with HCl (2 M) until the pH=2-3, and extracted with ethyl acetate (3×30 mL). The combined organic layers were washed with brine (2×20 mL) and dried over anhydrous sodium sulfate, filtered and the filtrate was concentrated in vacuo to give the title compound (650 mg) as a yellow solid. LC-MS (ESI⁺) m/z 522.7 (M+H)⁺.

### (R)-(1-(cyclopropylmethyl)-7-(3-ethylpyridin-4-yl)-3-fluoro-2-(piperidin-3-yl)-1H-indol-5-yl)(1-methylpyrrolo[3,4-c]pyrazol-5(1H,4H,6H)-yl)methanone (Intermediate UT)

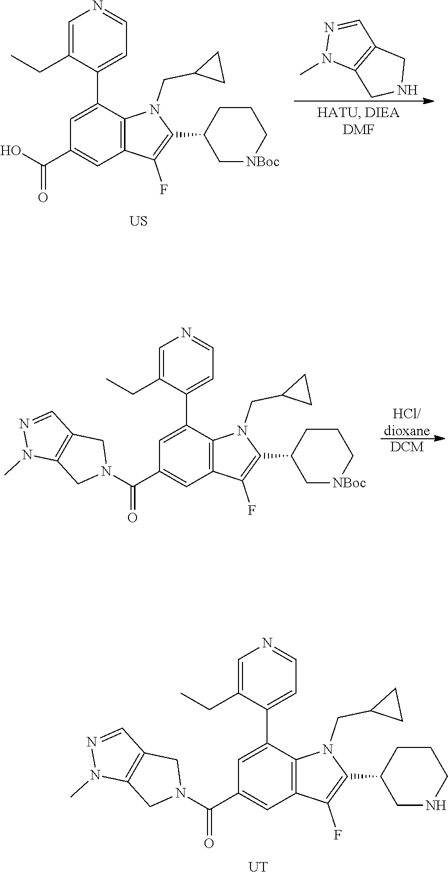

### Step 1—(R)-tert-butyl 3-(1-(cyclopropylmethyl)-7-(3-ethylpyridin-4-yl)-3-fluoro-5-(1-methyl-1,4,5,6-tetrahydropyrrolo[3,4-c]pyrazole-5-carbonyl)-1H-indol-2-yl)piperidine-1-carboxylate

To a solution of (R)-2-(1-(tert-butoxycarbonyl)piperidin-3-yl)-1-(cyclopropylmethyl)-7-(3-ethylpyridin-4-yl)-3-fluoro-1H-indole-5-carboxylic acid (600 mg, 1.15 mmol, Intermediate US) in DMF (8 mL) was added HATU (569 mg, 1.50 mmol) and DIEA (743 mg, 5.75 mmol). After addition, the mixture was stirred at 25° C. for 10 min, and then 1-methyl-1,4,5,6-tetrahydropyrrolo[3,4-c]pyrazole (202 mg, 1.27 mmol) was added. The resulting mixture was stirred at 25° C. for 20 min. On completion, the reaction mixture was diluted with water (20 mL) and extracted with ethyl acetate (3×10 mL). The combined organic layers were washed with brine (2×10 mL) and dried over anhydrous sodium sulfate, filtered and the filtrate was concentrated in vacuo to give the title compound (700 mg) as a yellow solid. LC-MS (ESI⁺) m/z 627.4 (M+H)⁺.

### Step 2—(R)-(1-(cyclopropylmethyl)-7-(3-ethylpyridin-4-yl)-3-fluoro-2-(piperidin-3-yl)-1H-indol-5-yl)(1-methylpyrrolo[3,4-c]pyrazol-5(1H,4H,6H)-yl)methanone

To a solution of (R)-tert-butyl 3-(1-(cyclopropylmethyl)-7-(3-ethylpyridin-4-yl)-3-fluoro-5-(1-methyl-1,4,5,6-tetrahydropyrrolo[3,4-c]pyrazole-5-carbonyl)-1H-indol-2-yl)piperidine-1-carboxylate (700 mg, 1.12 mmol) in DCM (5 mL) was added HCl/dioxane (2 M, 5 mL) and the mixture was stirred at 25° C. for 1 hr. On completion, the reaction mixture was concentrated in vacuo to give the title compound (600 mg, crude) as a yellow solid. LC-MS (ESI⁺) m/z 527.2 (M+H)⁺.

### (7-(6-Ethyl-2-methylpyridin-3-yl)-1-isobutyl-2-(1,2,5,6-tetrahydropyridin-3-yl)-1H-indol-5-yl)(4-(5-fluoro-3-methoxypyridin-2-yl)piperazin-1-yl)methanone (Intermediate UU)

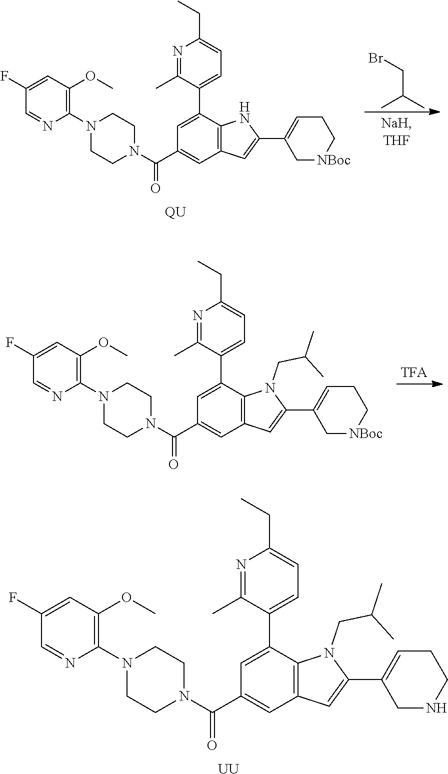

### Step 1—Tert-butyl 3-(7-(6-ethyl-2-methylpyridin-3-yl)-5-(4-(5-fluoro-3-methoxypyridin-2-yl)piperazine-1-carbonyl)-1-isobutyl-1H-indol-2-yl)-5,6-dihydropyridine-coup1(2H)-carboxylate

To a solution of tert-butyl 5-[7-(6-ethyl-2-methyl-3-pyridyl)-5-[4-(5-fluoro-3-methoxy-2-pyridyl)piperazine-1-carbonyl]-1H-indol-2-yl]-3,6-dihydro-2H-pyridine-1-carboxylate (270 mg, 412 gmol, Intermediate QU) in THF (4 mL) was added NaH (24.7 mg, 618 μmol, 60% dispersion in mineral oil) at 25° C. for 1 hr. Next, 1-bromo-2-methyl-propane (84.7 mg, 618 μmol, 67.2 μL) was added and the mixture was stirred at 25° C. for 11 hrs. On completion, the reaction mixture was quenched with NH₄Cl (10 mL) at 5° C., and then concentrated under reduced pressure to give a residue. The crude product was purified by reversed-phase HPLC (0.1% FA condition) to give the title compound (180 mg, 56% yield, FA) as a white solid. LC-MS (ESI+) m/z 711.4 (M+H)⁺; ¹H NMR (400 MHz, DMSO-d6) δ=7.82-7.68 (m, 3H), 7.35 (dd, J=2.4, 10.4 Hz, 1H), 7.24 (d, J=7.8 Hz, 1H), 6.96 (d, J=1.6 Hz, 1H), 6.61 (s, 1H), 6.03 (s, 1H), 3.83 (s, 3H), 3.70-3.53 (m, 11H), 3.24 (s, 5H), 2.79 (q, J=7.2 Hz, 3H), 2.28 (d, J=3.2 Hz, 2H), 1.41 (s, 9H), 1.26 (t, J=7.6 Hz, 4H), 0.21 (dd, J=6.4, 16.0 Hz, 6H).

### Step 2—(7-(6-Ethyl-2-methylpyridin-3-yl)-1-isobutyl-2-(1,2,5,6-tetrahydropyridin-3-yl)-1H-indol-5-yl)(4-(5-fluoro-3-methoxypyridin-2-yl)piperazin-1-yl)methanone

A solution of tert-butyl 5-[7-(6-ethyl-2-methyl-3-pyridyl)-5-[4-(5-fluoro-3-methoxy-2-pyridyl)piperazine-1-carbonyl]-1-isobutyl-indol-2-yl]-3,6-dihydro-2H-pyridine-1-carboxylate (180 mg, 253 μmol) in TFA (1 mL) was stirred at 25° C. for 0.5 hr. On completion, the reaction mixture was concentrated under reduced pressure to give the title compound (160 mg, 80% yield, TFA) as a yellow oil. LC-MS (ESI+) m/z 611.3 (M+H)⁺.

### [7-(2-Ethyl-6-methyl-3-pyridyl)-2-(1,2,3,6-tetrahydropyridin-5-yl)-1H-indol-5-yl]-[4-(5-fluoro-3-methoxy-2-pyridyl)piperazin-1-yl]methanone (Intermediate UV)

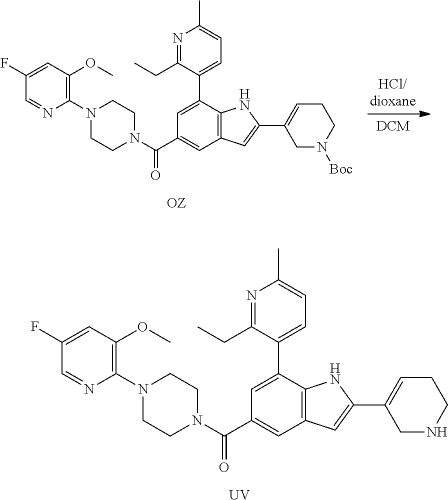

A solution of tert-butyl 5-[7-(2-ethyl-6-methyl-3-pyridyl)-5-[4-(5-fluoro-3-methoxy-2-pyridyl) piperazine-1-carbonyl]-1H-indol-2-yl]-3,6-dihydro-2H-pyridine-1-carboxylate (300 mg, 458 gmol, Intermediate OZ) in HCl/dioxane (2 mL) was stirred at 25° C. for 0.2 hr. On completion, The reaction was concentrated in vacuo to give the title compound (270 mg, quant. yield, HCl) as yellow solid. LC-MS (ESI⁺) m/z 555.3 (M+H)⁺; ¹H NMR (400 MHz, DMSO-d₆) δ 11.14 (s, 1H), 8.42-8.27 (m, 1H), 7.87 (d, J=7.6 Hz, 1H), 7.77 (d, J=8.4 Hz, 2H), 7.41-7.34 (m, 1H), 7.14 (s, 1H), 6.73 (s, 1H), 6.61 (s, 1H), 4.46-4.42 (m, 4H), 3.98 (s, 2H), 3.84 (s, 3H), 3.76-3.60 (m, 6H), 3.25 (s, 4H), 2.90 (s, 3H), 1.59 (s, 1H), 1.03 (t, J=7.2 Hz, 3H).

### 1-[5-[7-(2-Ethyl-6-methyl-3-pyridyl)-5-[4-(5-fluoro-3-methoxy-2-pyridyl)piperazine-1-carbonyl]-1H-indol-2-yl]-3,6-dihydro-2H-pyridin-1-yl]ethanone (Intermediate UW)

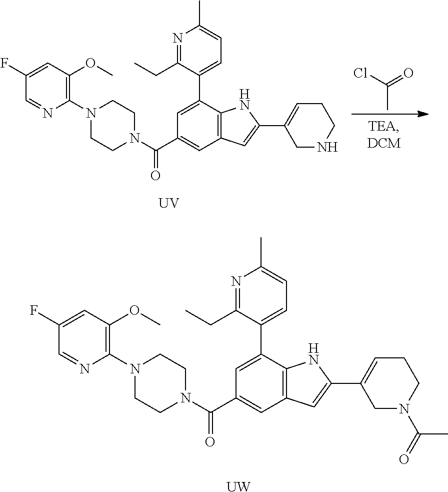

To a solution of [7-(2-ethyl-6-methyl-3-pyridyl)-2-(1,2,3,6-tetrahydropyridin-5-yl)-1H-indol-5-yl]-[4-(5-fluoro-3-methoxy-2-pyridyl)piperazin-1-yl]methanone (270 mg, 456 μmol, HCl, Intermediate UV) in DCM (5 mL) was added TEA (231 mg, 2.28 mmol, 317 μL) and acetyl chloride (53.7 mg, 685 μmol, 48.7 μL) at 0° C., then the reaction was stirred at 25° C. for 2 hrs. On completion, the reaction was quenched with MeOH (0.5 mL) and concentrated in vacuo. The crude product was purified by column chromatography (SiO₂, DCM:MeOH=30:1 to 10:1, Rf=0.5) to give the title compound (270 mg, 99% yield) as yellow solid. LC-MS (ESI⁺) m/z 597.4 (M+H)⁺; ¹H NMR (400 MHz, DMSO-d₆) δ 10.88 (d, J=5.6 Hz, 1H), 7.84 (d, J=2.0 Hz, 1H), 7.69 (s, 1H), 7.57 (d, J=7.6 Hz, 1H), 7.41 (d, J=8.8 Hz, 1H), 7.24 (d, J=7.6 Hz, 1H), 6.97 (s, 1H), 6.66 (s, 1H), 4.40 (s, 2H), 3.89 (s, 3H), 3.81-3.57 (m, 8H), 3.30 (s, 4H), 2.61 (s, 3H), 2.37 (s, 2H), 2.21-2.08 (m, 4H), 1.09 (t, J=7.2 Hz, 3H).

### Tert-butyl 3-(bromomethyl)pyrrolidine-1-carboxylate (CAS #1067230-65-2) (Intermediate UX)

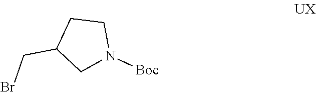 [SMILES: CC(C)(C)OC(=O)N1CCC(CBr)C1]

### 2-Bromo-1-ethyl-imidazole (CAS #663171-12-8) (Intermediate UY)

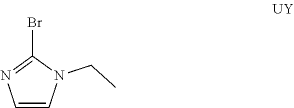

### Methyl 4-bromo-2-(1,2,3,6-tetrahydropyridin-5-yl)benzothiophene-6-carboxylate (Intermediate UZ)

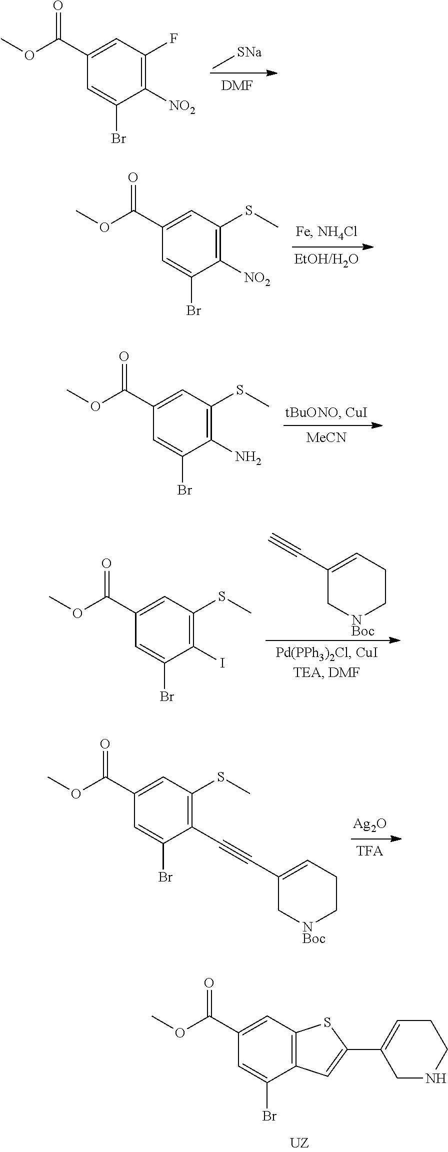

### Step 1—Methyl 3-bromo-5-methylsulfanyl-4-nitro-benzoate

To a solution of methyl 3-bromo-5-fluoro-4-nitro-benzoate (16 g, 60 mmol, CAS #1123171-93-6) in DMF (160 mL) was degassed and purged with N₂ three times. Then methylsulfanylsodium (19.21 g, 57.55 mmol, 17.46 mL, CAS #5188-07-8) was added to the mixture dropwise at 25° C. and the mixture was stirred at 25° C. for 3 hrs under N₂ atmosphere. On completion, the reaction mixture was diluted with H₂O (300 mL) and extracted with EtOAc (150 mL×2). The combined organic layers were washed with NaClO (50 mL), dried over Na₂SO₄, filtered and concentrated under reduced pressure to give a residue. The residue was purified by flash silica gel chromatography (ISCO®; 120 g SepaFlash® Silica Flash Column, Eluent of 0-10% EtOAc/PE gradient @100 mL/min) to give the title compound (2 g, 11% yield) as a yellow solid. ¹H NMR (400 MHz, DMSO-d₆) δ=8.10 (d, J=1.2 Hz, 1H), 8.02 (d, J=1.2 Hz, 1H), 3.91 (s, 3H), 2.65 (s, 3H).

### Step 2—Methyl 4-amino-3-bromo-5-methylsulfanyl-benzoate

To a solution of methyl 3-bromo-5-methylsulfanyl-4-nitro-benzoate (2 g, 7 mmol) in EtOH (20 mL) and H₂O (4 mL) was added Fe (1.82 g, 32.7 mmol) and NH₄Cl (3.49 g, 65.3 mmol). Then the mixture was stirred at 80° C. for 1 hr. On completion, the reaction mixture was filtered and the filtrate was concentrated under reduced pressure to give the title compound (1.8 g, 95% yield) as a yellow gum. LC-MS (ESI⁺) m/z 275.8/277.9 (M+1/M+2+1)⁺; ¹H NMR (400 MHz, DMSO-d₆) δ=7.86 (d, J=2.0 Hz, 1H), 7.78 (d, J=2.0 Hz, 1H), 6.04 (s, 2H), 3.78 (s, 3H), 2.40 (s, 3H).

### Step 3—Methyl 3-bromo-4-iodo-5-methylsulfanyl-benzoate

To a solution of tert-butyl nitrite (1.01 g, 9.78 mmol, 1.16 mL) and CuI (3.72 g, 19.6 mmol) in ACN (20 mL) was added a solution of methyl 4-amino-3-bromo-5-methylsulfanyl-benzoate (1.8 g, 6.5 mmol) in ACN (10 mL). The mixture was then stirred at 25° C. for 12 hrs. On completion, the reaction mixture was diluted with H₂O (30 mL) and extracted with EtOAc (20 mL×2). The combined organic layers were dried over Na₂SO₄, filtered and concentrated under reduced pressure to give a residue. The residue was purified by flash silica gel chromatography (ISCO®; 20 g SepaFlash® Silica Flash Column, Eluent of 0˜20% EtOAc/PE gradient @100 mL/min) to give the title compound (1.5 g, 48% yield) as a yellow solid. LC-MS (ESI⁺) m/z 386.7/388.8 (M+1/M+2+1); ¹H NMR (400 MHz, DMSO-d₆) δ=7.90 (d, J=1.6 Hz, 1H), 7.53 (d, J=1.6 Hz, 1H), 3.87 (s, 3H), 2.53 (s, 3H).

### Step 4—tert-butyl 5-[2-(2-bromo-4-methoxycarbonyl-6-methylsulfanyl-phenyl)ethynyl]-3,6-dihydro-2H-pyridine-1-carboxylate

A mixture of methyl 3-bromo-4-iodo-5-methylsulfanyl-benzoate (1.5 g, 3.9 mmol), tert-butyl 5-ethynyl-3,6-dihydro-2H-pyridine-1-carboxylate (803 mg, 3.88 mmol), Pd(PPh₃)₂Cl₂ (272.03 mg, 387.57 μmol), CuI (147.62 mg, 775.13 μmol) and TEA (1.18 g, 11.63 mmol, 1.62 mL) in DMF (20 mL) was degassed and purged with N₂ three times. Then the mixture was stirred at 90° C. for 2 hrs under N₂ atmosphere. On completion, the reaction mixture was diluted with H₂O (30 mL) and extracted with EtOAc (20 mL×2). The combined organic layers were washed with brine (50 mL), dried over Na₂SO₄, filtered and concentrated under reduced pressure to give a residue. The residue was purified by flash silica gel chromatography (ISCO®; 20 g SepaFlash® Silica Flash Column, Eluent of 0˜20% EtOAc/PE gradient @80 mL/min) and further purified by prep-TLC (SiO₂, EtOAc/PE=1/5) to give the title compound (560 mg, 30% yield) as a yellow gum. LC-MS (ESI⁺) m/z 409.9/411.9 (M+1/M+2+1)⁺.

### Step 5—Methyl 4-bromo-2-(1,2,3,6-tetrahydropyridin-5-yl)benzothiophene-6-carboxylate

To a solution of tert-butyl 5-[2-(2-bromo-4-methoxycarbonyl-6-methylsulfanyl-phenyl)ethynyl]-3,6-dihydro-2H-pyridine-1-carboxylate (650 mg, 1.39 mmol) in TFA (2 mL) was added Ag₂O (16 mg, 69.68 μmol). The mixture was stirred at 20° C. for 0.5 hr. On completion, the reaction mixture was filtered and the filtrate was concentrated under reduced pressure to give the title compound (450 mg, 87% yield) as a yellow gum.

### Methyl 4-(4,4,5,5-tetramethyl-1,3,2-dioxaborolan-2-yl)-2-[1-[3-(triazol-1-yl)propanoyl]-3,6-dihydro-2H-pyridin-5-yl]benzothiophene-6-carboxylate (Intermediate VA)

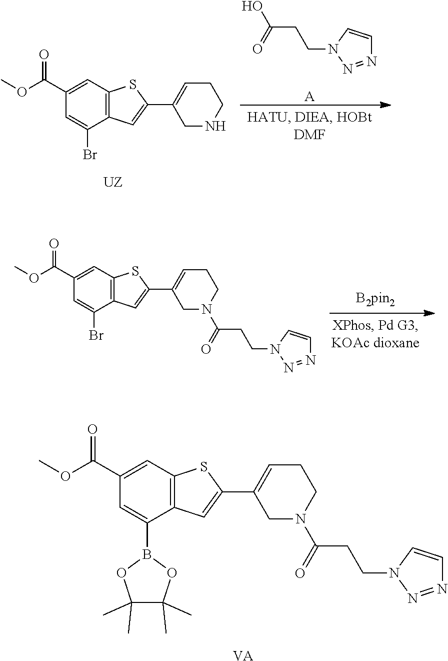

### Step 1—Methyl 4-bromo-2-[1-[3-(triazol-1-yl)propanoyl]-3,6-dihydro-2H-pyridin-5-yl]benzothiophene-6-carboxylate

To a solution of methyl 4-bromo-2-(1,2,3,6-tetrahydropyridin-5-yl)benzothiophene-6-carboxylate (400 mg, 1.14 mmol, Intermediate UZ), 3-(triazol-1-yl)propanoic acid (160 mg, 1.14 mmol, Intermediate A), HOBt (307 mg, 2.27 mmol) and DIEA (881 mg, 6.81 mmol, 1.19 mL) in DMF (4 mL) was added and HATU (518 mg, 1.36 mmol). The mixture was then stirred at 20° C. for 0.5 hr. On completion, the reaction mixture was diluted with H₂O (30 mL) and extracted with EtOAc (20 mL×2). The combined organic layers were washed with brine (50 mL), dried over Na₂SO₄, filtered and concentrated under reduced pressure to give a residue. The residue was purified by flash silica gel chromatography (ISCO®; 20 g SepaFlash® Silica Flash Column, Eluent of 0˜100% EtOAc/PE gradient @100 mL/min) to give the title compound (380 mg, 67% yield) as a yellow gum. LC-MS (ESI⁺) m/z 474.9/476.9 (M+1/M+2+1)⁺; ¹H NMR (400 MHz, DMSO-d₆) δ=8.59 (br d, J=2.4 Hz, 1H), 8.19-7.92 (m, 2H), 7.69 (br s, 1H), 7.55-7.30 (m, 1H), 6.62-6.46 (m, 1H), 4.64 (br s, 2H), 4.55-4.39 (m, 2H), 3.94-3.83 (m, 3H), 3.60 (br d, J=5.6 Hz, 2H), 3.20-3.14 (m, 2H), 2.40-2.28 (m, 2H).

### Step 2—Methyl 4-(4,4,5,5-tetramethyl-1,3,2-dioxaborolan-2-yl)-2-[1-[3-(triazol-1-yl)propanoyl]-3,6-dihydro-2H-pyridin-5-yl]benzothiophene-6-carboxylate

A mixture of methyl 4-bromo-2-[1-[3-(triazol-1-yl)propanoyl]-3,6-dihydro-2H-pyridin-5-yl]benzothiophene-6-carboxylate (350 mg, 736 μmol), 4,4,5,5-tetramethyl-2-(4,4,5,5-tetramethyl-1,3,2-dioxaborolan-2-yl)-1,3,2-dioxaborolane (374 mg, 1.47 mmol), KOAc (217 mg, 2.21 mmol) and XPhos Pd G₃ (62.32 mg, 73.63 μmol) in dioxane (20 mL) was degassed and purged with N₂ three times. Then the mixture was stirred at 80° C. for 12 hrs under N₂ atmosphere. On completion, the reaction mixture was diluted with H₂O (30 mL) and extracted with EtOAc (20 mL×2). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 0˜100% EtOAc/PE gradient @100 mL/min) to give the title compound (250 mg, 59% yield) as a yellow gum. LC-MS (ESI⁺) m/z 523.1 (M+1)⁺.

### 4-(2-Ethyl-6-methyl-3-pyridyl)-2-[1-[3-(triazol-1-yl)propanoyl]-3,6-dihydro-2H-pyridin-5-yl]benzothiophene-6-carboxylic acid (Intermediate VB)

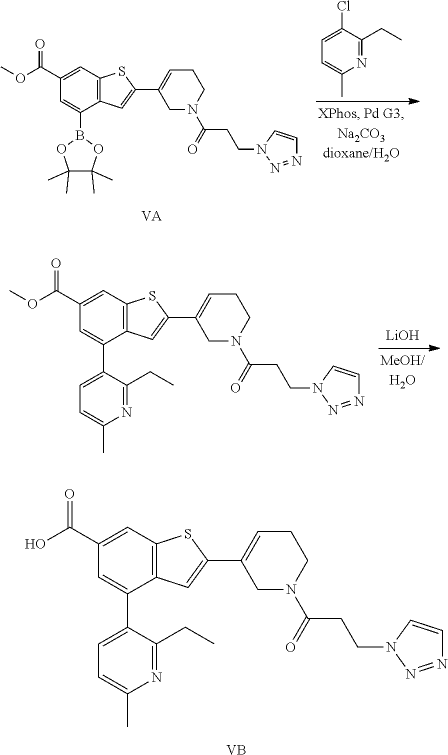

### Step 1—Methyl 4-(2-ethyl-6-methyl-3-pyridyl)-2-[1-[3-(triazol-1-yl)propanoyl]-3,6-dihydro-2H-pyridin-5-yl]benzothiophene-6-carboxylate

A mixture of 3-chloro-2-ethyl-6-methyl-pyridine (104 mg, 669.96 μmol), methyl 4-(4,4,5,5-tetramethyl-1,3,2-dioxaborolan-2-yl)-2-[1-[3-(triazol-1-yl)propanoyl]-3,6-dihydro-2H-pyridin-5-yl]benzothiophene-6-carboxylate (350 mg, 669.96 μmol, Intermediate VA), K₂CO₃ (277.78 mg, 2.01 mmol) and XPhos Pd G₃ (57 mg, 67.00 μmol) in dioxane (5 mL) and H₂O (1 mL) was degassed and purged with N₂ three times. Then the mixture was stirred at 80° C. for 1 hr under N₂ atmosphere. The reaction mixture was filtered and the filtrate was concentrated under reduced pressure to give a residue. The residue was purified by flash silica gel chromatography (ISCO®; 12 g SepaFlash® Silica Flash Column, Eluent of 0˜10% MeOH/DCM gradient @100 mL/min) to give the title compound (200 mg, 52% yield) as a brown gum. LC-MS (ESI⁺) m/z 516.1 (M+1)⁺.

### Step 2—4-(2-Ethyl-6-methyl-3-pyridyl)-2-[1-[3-(triazol-1-yl)propanoyl]-3,6-dihydro-2H-pyridin-5-yl]benzothiophene-6-carboxylic acid

To a solution of methyl 4-(2-ethyl-6-methyl-3-pyridyl)-2-[1-[3-(triazol-1-yl)propanoyl]-3,6-dihydro-2H-pyridin-5-yl]benzothiophene-6-carboxylate (200 mg, 387.88 μmol) in THF (2 mL) and H₂O (2 mL) was added LiOH H₂O (81 mg, 1.94 mmol). The mixture was stirred at 20° C. for 12 hrs. On completion, the mixture was acidified with HCl(2M) to adjust the pH=5. During this period, brown precipitate was formed, which was filtered to give the title compound. Then the filtrate was concentrated under reduced pressure to give additional title compound (180 mg, 83% yield) as a yellow gum. LC-MS (ESI⁺) m/z 502.1 (M+1)⁺.

### (R)-6-(1-(3-(1H-1,2,3-triazol-1-yl)propanoyl)-1,2,5,6-tetrahydropyridin-3-yl)-7-fluoro-4-(1-methylpyrrolidin-3-yl)-1H-indole-2-carboxylic acid (Intermediate VC)

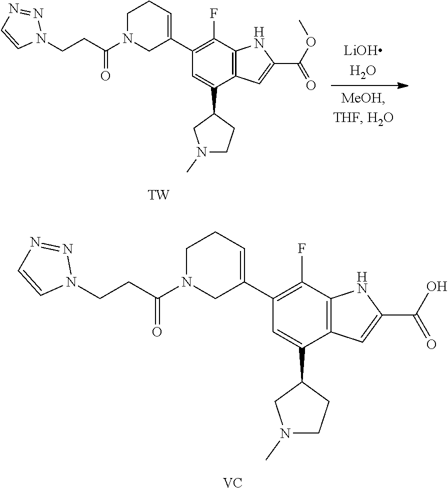 [SMILES: COC(=O)c1cc2c([C@H]3CCN(C)C3)cc(C3=CCCN(C(=O)CCn4ccnn4)C3)c(F)c2[nH]1 ; "TW" ; CN1CC[C@H](c2cc(C3=CCCN(C(=O)CCn4ccnn4)C3)c(F)c3[nH]c(C(=O)O)cc23)C1 ; "VC"]

A mixture of methyl 7-fluoro-4-[(3R)-1-methylpyrrolidin-3-yl]-6-[1-[3-(triazol-1-yl)propanoyl]-3,6-dihydro-2H-pyridin-5-yl]-1H-indole-2-carboxylate (154 mg, 320 μmol, Intermediate TW) and LiOH·H₂O (40.35 mg, 961.43 μmol) in THF (4 mL), MeOH (1 mL) and H₂O (1 mL) was degassed and purged with N₂ three times. Then the mixture was stirred at 25° C. for 12 hrs under N₂ atmosphere. On completion, the mixture was filtered and concentrated under reduced pressure to give the title compound (140 mg, 74% yield) as a white solid. LC-MS (ESI⁺) m/z 467.1 (M+H)⁺.

### Methyl 4-(1-tert-butoxycarbonyl-4-ethyl-pyrrolidin-3-yl)-6-chloro-7-fluoro-1H-indole-2-carboxylate (Intermediate VD)

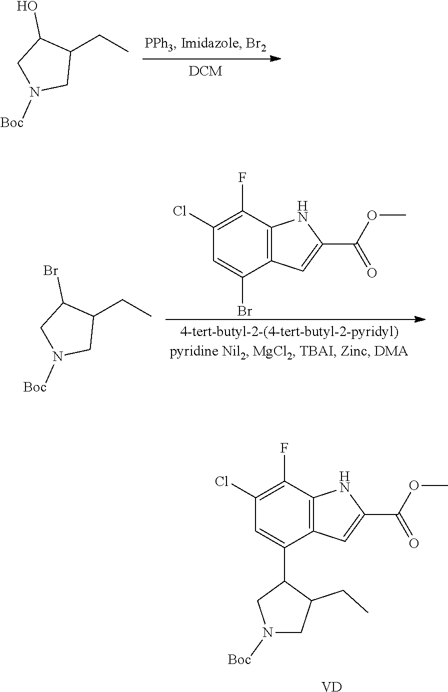 [SMILES: CCC1CN(C(=O)OC(C)(C)C)CC1O ; CCC1CN(C(=O)OC(C)(C)C)CC1Br ; COC(=O)c1cc2c(Br)cc(Cl)c(F)c2[nH]1 ; CCC1CN(C(=O)OC(C)(C)C)CC1c1cc(Cl)c(F)c2[nH]c(C(=O)OC)cc12 ; "VD"]

### Step 1—Tert-butyl 3-bromo-4-ethyl-pyrrolidine-1-carboxylate

To a solution of tert-butyl 3-ethyl-4-hydroxy-pyrrolidine-1-carboxylate (2.2 g, 10 mmol, synthesized via Step 1 of Intermediate OO), Imidazole (904 mg, 13.28 mmol) and PPh₃ (2.68 g, 10.2 mmol) in DCM (20 mL) was added Br₂ (2.12 g, 13.3 mmol, 684.39 μL) dropwise at 25° C. The mixture was then stirred at 25° C. for 12 hrs. On completion, the reaction mixture was diluted with H₂O (50 mL) and extracted with DCM (30 mL×2). The combined organic layers were dried over Na₂SO₄, filtered and concentrated under reduced pressure to give a residue. The residue was purified by flash silica gel chromatography (ISCO®; 40 g SepaFlash® Silica Flash Column, Eluent of 0˜30% Ethylacetate/Petroleum ether gradient @100 mL/min) to give the title compound (3.65 g, 61% yield) as a yellow gum. LC-MS (ESI⁺) m/z 279.0 (M+1)⁺; ¹H NMR (400 MHz, DMSO-d₆) δ=4.76 (t, J=3.6 Hz, 1H), 3.93-3.79 (m, 1H), 3.75-3.69 (m, 1H), 3.47-3.40 (m, 2H), 2.96-2.86 (m, 1H), 2.17-2.07 (m, 1H), 1.55-1.44 (m, 1H), 1.40 (d, J=4.8 Hz, 9H), 0.93-0.83 (m, 3H).

### Step 2—Methyl 4-(1-tert-butoxycarbonyl-4-ethyl-pyrrolidin-3-yl)-6-chloro-7-fluoro-1H-indole-2-carboxylate

A mixture of methyl 4-bromo-6-chloro-7-fluoro-1H-indole-2-carboxylate (4 g, 13.05 mmol, synthesized via Steps 1-5 of Intermediate C), tert-butyl 3-bromo-4-ethyl-pyrrolidine-1-carboxylate (3.63 g, 13.05 mmol), diiodonickel (816 mg, 2.61 mmol, 139.90 μL), dichloromagnesium (1.24 g, 13.05 mmol, 535.56 μL), 4-tert-butyl-2-(4-tert-butyl-2-pyridyl)pyridine (350 mg, 1.30 mmol) and TBAI (4.82 g, 13.05 mmol) in DMA (40 mL) was degassed and purged with N₂ three times and Zinc (2.88 g, 44.04 mmol) was added to the mixture under N₂ atmosphere. Then the mixture was stirred at 60° C. for 5 hrs under N₂ atmosphere. On completion, the reaction mixture was filtered and concentrated under reduced pressure to give a mixture. The reaction mixture was diluted with H₂O (50 mL) and extracted with EtOAc (30 mL×2). The combined organic layers were washed with brine (50 mL), dried over Na₂SO₄, filtered and concentrated under reduced pressure to give a residue. The residue was purified by flash silica gel chromatography (ISCO®; 40 g SepaFlash® Silica Flash Column, Eluent of 0˜30% EtOAc/PE gradient @100 mL/min) to give the title compound (900 mg) as a yellow gum. LC-MS (ESI⁺) m/z 369.0 (M−56+1)⁺.

### 4-(1-Tert-butoxycarbonyl-4-ethyl-pyrrolidin-3-yl)-7-fluoro-6-[1-[3-(triazol-1-yl)propanoyl]-3,6-dihydro-2H-pyridin-5-yl]-1H-indole-2-carboxylic acid (Intermediate VE)

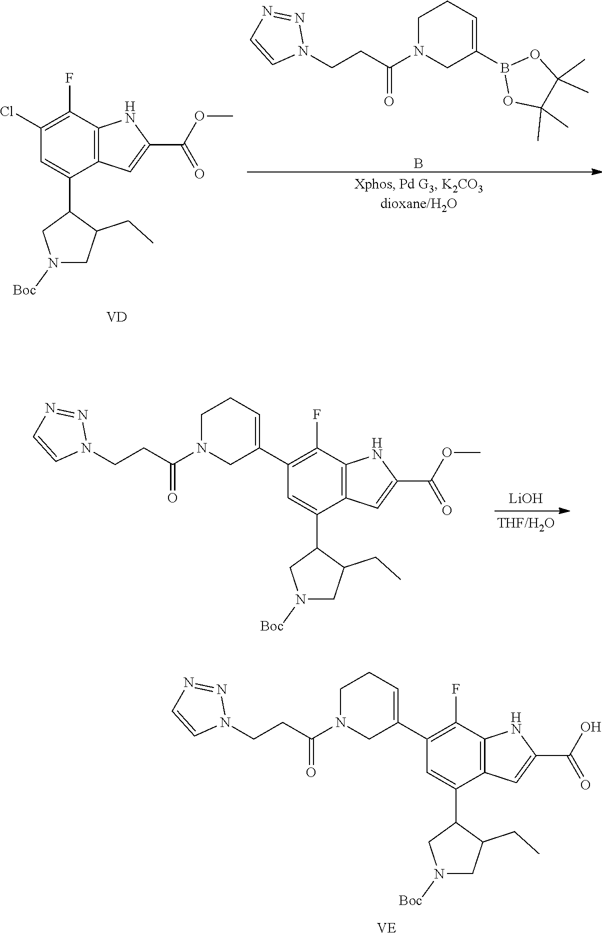

### Step 1—Methyl 4-(1-tert-butoxycarbonyl-4-ethyl-pyrrolidin-3-yl)-7-fluoro-6-[1-[3-(triazol-1-yl)propanoyl]-3,6-dihydro-2H-pyridin-5-yl]-1H-indole-2-carboxylate

A mixture of methyl 4-(1-tert-butoxycarbonyl-4-ethyl-pyrrolidin-3-yl)-6-chloro-7-fluoro-1H-indole-2-carboxylate (900 mg, 2.12 mmol, Intermediate VD), 1-[5-(4,4,5,5-tetramethyl-1,3,2-dioxaborolan-2-yl)-3,6-dihydro-2H-pyridin-1-yl]-3-(triazol-1-yl)propan-1-one (704 mg, 2.12 mmol, Intermediate B), K₂CO₃ (878 mg, 6.35 mmol) and XPhos Pd G₃ (179 mg, 211.82 μmol) in dioxane (10 mL) and H₂O (2 mL) was degassed and purged with N₂ three times. Then the mixture was stirred at 80° C. for 2 hrs under N₂ atmosphere. On completion, the reaction mixture was diluted with H₂O (50 mL) and extracted with EtOAc (30 mL×2). The combined organic layers were dried over Na₂SO₄, filtered and concentrated under reduced pressure to give a residue. The residue was purified by flash silica gel chromatography (ISCO®; 20 g SepaFlash® Silica Flash Column, Eluent of 0˜50% EtOAc/PE gradient @100 mL/min) to give the title compound (900 mg, 64% yield) as a yellow gum. LC-MS (ESI⁺) m/z 595.3 (M+1); ¹H NMR (400 MHz, DMSO-d₆) δ=12.45 (br s, 1H), 8.12-8.04 (m, 1H), 7.70-7.62 (m, 1H), 7.33 (br s, 1H), 6.93 (br dd, J=5.2, 17.2 Hz, 1H), 6.09 (br d, J=3.6 Hz, 1H), 4.59 (q, J=6.4 Hz, 2H), 4.35-4.24 (m, 2H), 3.85 (s, 3H), 3.67 (br d, J=7.2 Hz, 1H), 3.63-3.53 (m, 2H), 3.41-3.35 (m, 2H), 3.10-3.04 (m, 2H), 3.00-2.90 (m, 1H), 2.42-2.35 (m, 1H), 2.32-2.21 (m, 2H), 1.38 (br d, J=16.8 Hz, 9H), 1.32-1.21 (m, 2H), 0.78-0.68 (m, 3H).

### Step 2—4-(1-Tert-butoxycarbonyl-4-ethyl-pyrrolidin-3-yl)-7-fluoro-6-[1-[3-(triazol-1-yl)propanoyl]-3,6-dihydro-2H-pyridin-5-yl]-1H-indole-2-carboxylic acid

To a solution of methyl 4-(1-tert-butoxycarbonyl-4-ethyl-pyrrolidin-3-yl)-7-fluoro-6-[1-[3-(triazol-1-yl)propanoyl]-3,6-dihydro-2H-pyridin-5-yl]-1H-indole-2-carboxylate (900 mg, 1.51 mmol) in THF (4 mL) and H₂O (4 mL) was added LiOH H₂O (317.52 mg, 7.57 mmol). The mixture was stirred at 20° C. for 12 hrs. On completion, the mixture was acidified with HCl (2M) to adjust pH=5. During this period, brown precipitate was formed, which filtered and dried under reduced pressure to give the title compound (830 mg, 85% yield) as a brown solid. LC-MS (ESI⁺) m/z 518.3 (M+1)⁺.

### 2-(1-(Tert-butoxycarbonyl)-1,2,5,6-tetrahydropyridin-3-yl)-7-(6-ethyl-2-methylpyridin-3-yl)-1-isobutyl-1H-indole-5-carboxylic acid (Intermediate VF)

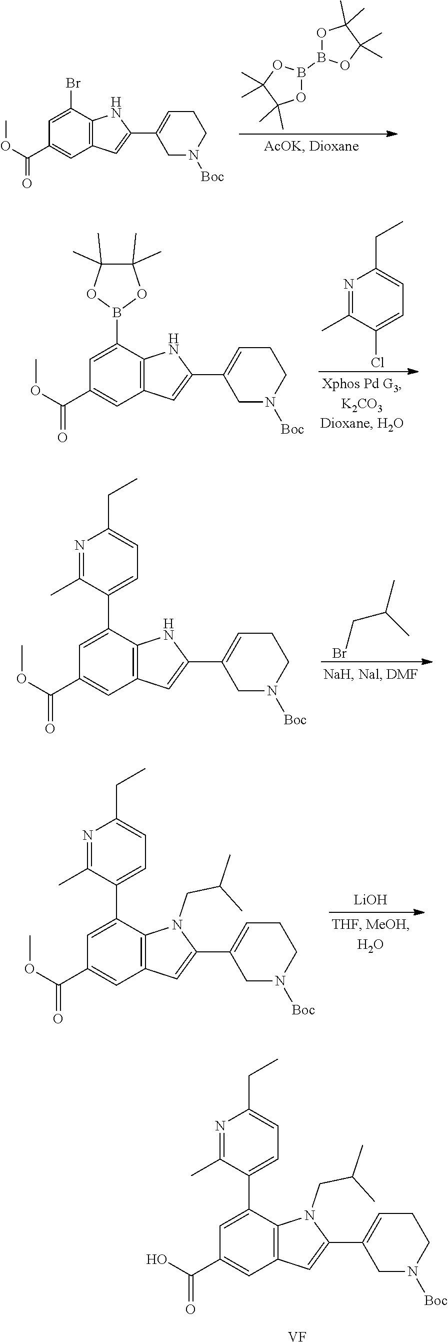

### Step 1—Methyl 2-(1-(tert-butoxycarbonyl)-1,2,5,6-tetrahydropyridin-3-yl)-7-(4,4,5,5-tetramethyl-1,3,2-dioxaborolan-2-yl)-1H-indole-5-carboxylate

A mixture of methyl 7-bromo-2-(1-tert-butoxycarbonyl-3,6-dihydro-2H-pyridin-5-yl)-1H-indole-5-carboxylate (1 g, 2.30 mmol, synthesized via Steps 1-2 from Intermediate NM), 4,4,5,5-tetramethyl-2-(4,4,5,5-tetramethyl-1,3,2-dioxaborolan-2-yl)-1,3,2-dioxaborolane (700 mg, 2.76 mmol), Pd(dppf)Cl₂ (168 mg, 230 μmol), and AcOK (676 mg, 6.89 mmol) in dioxane (10 mL) was degassed and purged with N₂ three times at 20° C. Then the mixture was stirred at 80° C. for 1 hr under N₂ atmosphere. On completion, the reaction mixture was concentrated under reduced pressure to remove solvent. The residue was purified by column chromatography (SiO₂, Petroleum ether:Ethyl acetate=0/1 to 10/1) to give the compound (800 mg, 72% yield) as a yellow solid. LC-MS (ESI⁺) m/z 483.3 (M+H)⁺.

### Step 2—Methyl 2-(1-(tert-butoxycarbonyl)-1,2,5,6-tetrahydropyridin-3-yl)-7-(6-ethyl-2-methylpyridin-3-yl)-1H-indole-5-carboxylate

A mixture of methyl 2-(1-tert-butoxycarbonyl-3,6-dihydro-2H-pyridin-5-yl)-7-(4,4,5,5-tetramethyl-1,3,2-dioxaborolan-2-yl)-1H-indole-5-carboxylate (800 mg, 1.66 mmol), 3-chloro-6-ethyl-2-methyl-pyridine (258 mg, 1.66 mmol, Intermediate QH), XPhos Pd G₃ (140 mg, 166 μmol), and K₂CO₃ (688 mg, 4.98 mmol) in dioxane (10 mL) and H₂O (2 mL) was degassed and purged with N₂ three times at 20° C. Then the mixture was stirred at 80° C. for 1 hr under N₂ atmosphere. On completion, the reaction mixture was concentrated under reduced pressure to remove solvent. The residue was purified by column chromatography (SiO₂, Petroleum ether:Ethyl acetate=0/1 to 10/1) to give the title compound (600 mg, 76% yield) as a yellow solid. LC-MS (ESI⁺) m/z 476.7 (M+H)⁺; ¹H NMR (400 MHz, DMSO-d6) δ=11.07-10.98 (m, 1H), 8.27-8.19 (m, 1H), 7.58-7.52 (m, 1H), 7.45 (d, J=1.5 Hz, 1H), 7.22-7.17 (m, 1H), 6.70-6.61 (m, 2H), 4.23 (s, 2H), 3.86-3.80 (m, 3H), 3.48-3.44 (m, 2H), 2.85-2.78 (m, 1H), 2.26-2.22 (m, 2H), 2.22-2.18 (m, 3H), 1.45-1.40 (m, 9H), 1.34-1.29 (m, 3H).

### Step 3—Methyl 2-(1-(tert-butoxycarbonyl)-1,2,5,6-tetrahydropyridin-3-yl)-7-(6-ethyl-2-methylpyridin-3-yl)-1-isobutyl-1H-indole-5-carboxylate

To a solution of methyl 2-(1-tert-butoxycarbonyl-3,6-dihydro-2H-pyridin-5-yl)-7-(6-ethyl-2-methyl-3-pyridyl)-1H-indole-5-carboxylate (600 mg, 1.26 mmol) and NaI (18.9 mg, 126 μmol) in DMF (5 mL) was added NaH (101 mg, 2.52 mmol, 60% dispersion in mineral oil) under N₂ atmosphere at 0° C., and the mixture was stirred at 0° C. for 1 hr under N₂ atmosphere. Then 1-bromo-2-methyl-propane (190 mg, 1.39 mmol, 151 mL) was added dropwise at 0° C. The resulting mixture was stirred at 20° C. for 11 hrs. On completion, the reaction mixture was quenched with water (20 mL) and extracted with ethyl acetate (3×20 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=20/1 to 1/1) to give the title compound (120 mg, 18% yield) as a yellow solid. LC-MS (ESI⁺) m/z 532.3 (M+H)⁺; ¹H NMR (400 MHz, DMSO-d6) δ=8.26 (s, 1H), 7.77-7.67 (m, 1H), 7.52-7.40 (m, 1H), 7.30-7.19 (m, 1H), 6.78-6.66 (m, 1H), 6.11-5.97 (m, 1H), 3.84 (s, 2H), 3.66-3.52 (m, 2H), 3.25-3.14 (m, 3H), 2.87-2.76 (m, 2H), 2.28 (s, 2H), 2.19-2.09 (m, 3H), 1.41 (s, 9H), 1.35-1.21 (m, 6H), 0.29-0.13 (m, 6H).

### Step 4—2-(1-(Tert-butoxycarbonyl)-1,2,5,6-tetrahydropyridin-3-yl)-7-(6-ethyl-2-methylpyridin-3-yl)-1-isobutyl-1H-indole-5-carboxylic acid

To a solution of methyl 2-(1-tert-butoxycarbonyl-3,6-dihydro-2H-pyridin-5-yl)-7-(6-ethyl-2-methyl-3-pyridyl)-1-isobutyl-indole-5-carboxylate (120 mg, 226 μmol) in MeOH (1 mL), THF (1 mL) and H₂O (1 mL) was added LiOH (21.6 mg, 903 μmol) and the mixture was stirred at 40° C. for 1 hr. On completion, the reaction mixture was diluted with water (10 mL) and extracted with ethyl acetate (3×10 mL). The combined organic layers were washed with brine (10 mL) and dried over anhydrous sodium sulfate, filtered and the filtrate was concentrated in vacuo to give the title compound (100 mg, 86% yield) as a yellow solid. LC-MS (ESI⁺) m/z 518.8 (M+H)⁺.

### (7-(6-Ethyl-2-methylpyridin-3-yl)-1-isobutyl-2-(1,2,5,6-tetrahydropyridin-3-yl)-1H-indol-5-yl)(1-methylpyrrolo[3,4-c]pyrazol-5(1H,4H,6H)-yl)methanone (Intermediate VG)

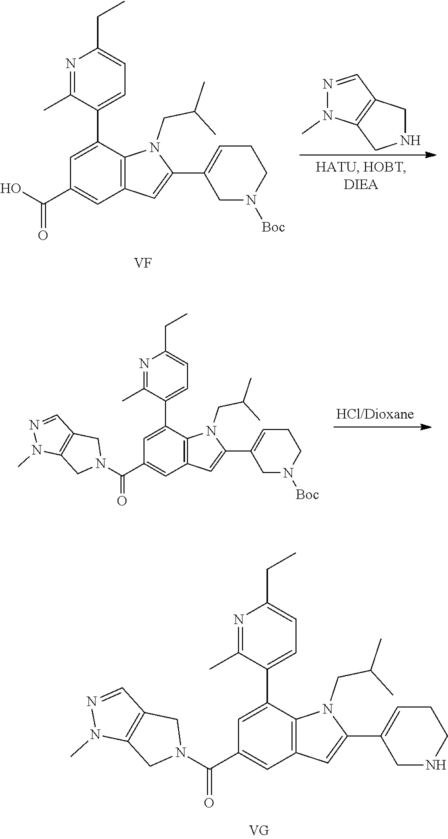

### Step 1—Tert-butyl 3-(7-(6-ethyl-2-methylpyridin-3-yl)-1-isobutyl-5-(1-methyl-1,4,5,6-tetrahydropyrrolo[3,4-c]pyrazole-5-carbonyl)-1H-indol-2-yl)-5,6-dihydropyridine-1(2H)-carboxylate

To a solution of 2-(1-tert-butoxycarbonyl-3,6-dihydro-2H-pyridin-5-yl)-7-(6-ethyl-2-methyl-3-pyridyl)-1-isobutyl-indole-5-carboxylic acid (100 mg, 193 μmol, Intermediate VF) in DMF (1 mL) was added dropwise HATU (110 mg, 290 μmol), HOBt (52.2 mg, 386 μmol) and DIEA (125 mg, 966 μmol, 168 μL) at 25° C. After addition, the mixture was stirred at 0° C. for 10 mins, and then 1-methyl-5,6-dihydro-4H-pyrrolo[3,4-c]pyrazole (46.3 mg, 290 μmol, HCl, CAS #762233-62-5) was added dropwise at 0° C. Then the resulting mixture was stirred at 25° C. for 20 mins. On completion, the reaction mixture was diluted with water (10 mL) and extracted with ethyl acetate (3×10 mL). The combined organic layers were washed with brine (10 mL) and dried over anhydrous sodium sulfate, filtered and the filtrate was concentrated in vacuo to give the title compound (100 mg) as a yellow solid. LC-MS (ESI⁺) m/z 623.4 (M+H)⁺.

### Step 2—(7-(6-Ethyl-2-methylpyridin-3-yl)-1-isobutyl-2-(1,2,5,6-tetrahydropyridin-3-yl)-1H-indol-5-yl)(1-methylpyrrolo[3,4-c]pyrazol-5(1H,4H,6H)-yl)methanone

To a solution of tert-butyl 5-[7-(6-ethyl-2-methyl-3-pyridyl)-1-isobutyl-5-(1-methyl-4,6-dihydropyrrolo[3,4-c]pyrazole-5-carbonyl)indol-2-yl]-3,6-dihydro-2H-pyridine-1-carboxylate (100 mg, 161 μmol) in DCM (10 mL) was added HCl/dioxane (8 M, 2 mL). The mixture was stirred at 20° C. for 1 hr. On completion, the reaction mixture was concentrated under reduced pressure to remove solvent and give the title compound (100 mg, HCl) as a yellow solid. LC-MS (ESI⁺) m/z 523.2 (M+H)⁺.

### (1—(Cyclopropylmethyl)-7-(2-ethyl-6-methylpyridin-3-yl)-3-fluoro-2-(1,2,5,6-tetrahydropyridin-3-yl)-1H-indol-5-yl)(1-ethylpyrrolo[3,4-c]pyrazol-5(1H,4H,6H)-yl)methanone (Intermediate VH)

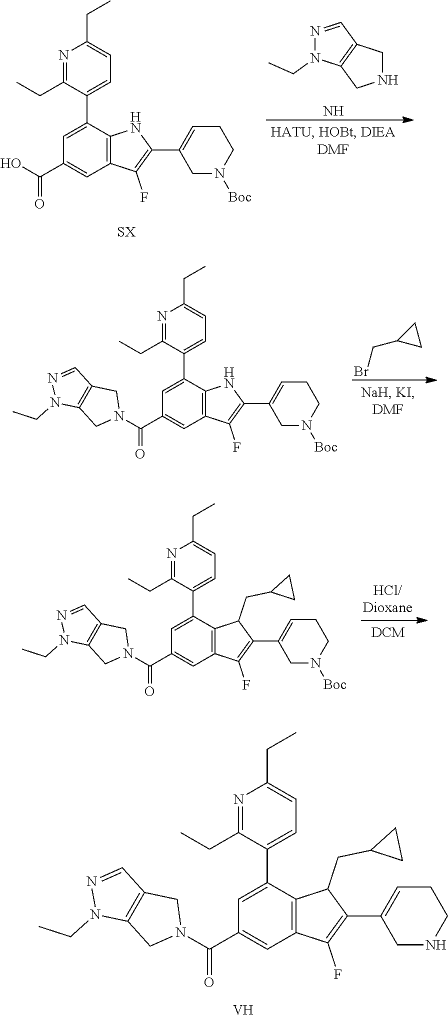

### Step 1—Tert-butyl 3-(5-(1-ethyl-1,4,5,6-tetrahydropyrrolo[3,4-c]pyrazole-5-carbonyl)-7-(2-ethyl-6-methylpyridin-3-yl)-3-fluoro-1H-indol-2-yl)-5,6-dihydropyridine-1(2H)-carboxylate

To a solution of 2-(1-(tert-butoxycarbonyl)-1,2,5,6-tetrahydropyridin-3-yl)-7-(2-ethyl-6-methylpyridin-3-yl)-3-fluoro-1H-indole-5-carboxylic acid (290 mg, 605 μmol, Intermediate SX) in DMF (3 mL) was added HATU (276 mg, 726 μmol), HOBt (123 mg, 907 μmol) and DIEA (391 mg, 3.02 mmol). After addition, the mixture was stirred at 25° C. for 10 min, and then 1-ethyl-1,4,5,6-tetrahydropyrrolo[3,4-c]pyrazole (124 mg, 907 μmol, Intermediate NH) was added. The resulting mixture was stirred at 25° C. for 20 min. On completion, the reaction mixture was diluted with water (20 mL) and extracted with ethyl acetate (3×10 mL). The combined organic layers were washed with brine (2×10 mL) and dried over anhydrous sodium sulfate, filtered and the filtrate was concentrated in vacuo to give the title compound (360 mg) as a yellow solid. LC-MS (ESI⁺) m/z 599.4 (M+H)⁺.

### Step 2—Tert-butyl 3-(1-(cyclopropylmethyl)-5-(1-ethyl-1,4,5,6-tetrahydropyrrolo[3,4-c]pyrazole-5-carbonyl)-7-(2-ethyl-6-methylpyridin-3-yl)-3-fluoro-1H-indol-2-yl)-5,6-dihydropyridine-1(2H)-carboxylate

To a solution of tert-butyl 3-(5-(1-ethyl-1,4,5,6-tetrahydropyrrolo[3,4-c]pyrazole-5-carbonyl)-7-(2-ethyl-6-methylpyridin-3-yl)-3-fluoro-1H-indol-2-yl)-5,6-dihydropyridine-1(2H)-carboxylate (360 mg, 601 μmol) in DMF (4 mL) was added NaH (48.1 mg, 1.20 mmol) at 0° C., and the mixture was stirred at this temperature for 1 hr. Then (bromomethyl)cyclopropane (122 mg, 902 μmol, CAS #7051-34-5) and KI (9.98 mg, 60.1 μmol) was added at 0° C. The resulting mixture was stirred at 25° C. for 2 hrs. On completion, the reaction mixture was quenched with NH₄Cl (10 mL) and extracted with ethyl acetate (3×10 mL). The combined organic layers were washed with brine (2×5 mL) and dried over anhydrous sodium sulfate, filtered and the filtrate was concentrated in vacuo to give the title compound (350 mg) as a brown solid. LC-MS (ESI⁺) m/z 653.3 (M+H)⁺.

### Step 3—(1-Cyclopropylmethyl)-7-(2-ethyl-6-methylpyridin-3-yl)-3-fluoro-2-(1,2,5,6-tetrahydropyridin-3-yl)-1H-indol-5-yl)(1-ethylpyrrolo[3,4-c]pyrazol-5(1H,4H,6H)-yl)methanone

To a solution of tert-butyl 3-(1-(cyclopropylmethyl)-5-(1-ethyl-1,4,5,6-tetrahydropyrrolo[3,4-c]pyrazole-5-carbonyl)-7-(2-ethyl-6-methylpyridin-3-yl)-3-fluoro-1H-indol-2-yl)-5,6-dihydropyridine-1(2H)-carboxylate (350 mg, 536 μmol) in DCM (2 mL) was added HCl/dioxane (2 M, 2 mL). The mixture was then stirred at 25° C. for 2 hrs. On completion, the reaction mixture was concentrated in vacuo to give the title compound (300 mg) as a yellow solid. LC-MS (ESI⁺) m/z 553.2 (M+H)⁺.

TABLE 4
Compounds synthesized via Method 1, coupling of the corresponding amines and carboxylic acids

| | | | LCMS | |
|---|---|---|---|---|
| | | | (ESI+) | |
| | | | m/z | |
| I-#^{a} | Amine | Acid | (M + H)⁺ | ¹H NMR (400 MHz, DMSO-d₆) δ |
| I-372 ^{b, c, d} | BU | BT | 473.2 | 8.36-8.14 (m, 1H), 7.76 (s, 1H), 7.68-7.46 (m, 2H), 7.35 (s, |
| | | | | 1H), 6.87-6.89 (m, 1H), 6.62-6.46 (m, 1H), 6.38 (d, J = 6.4 |
| | | | | Hz, 1H), 6.34-6.20 (m, 1H), 5.59-5.24 (m, 1H), 5.00-4.69 |
| | | | | (m, 2H), 4.67-4.12 (m, 4H), 3.80-3.61 (m, 2H), 3.15 (s, 6H), |
| | | | | 2.88-2.78 (m, 2H), 2.43-2.29 (m, 2H) |
| I-378 ^{e} | CF | D | 678.3 | 2.2-2.3 (m, 1 H), 2.3 (br s, 1 H), 3.0-3.1 (m, 4 H), 3.1-3.1 |
| | | | | (m, 2 H), 3.6 (br t, J = 5.2 Hz, 1 H), 3.7 (br s, 1 H), 3.7 (br s, 3 |
| | | | | H), 3.8-3.8 (m, 4 H), 4.3-4.4 (m, 2 H), 4.6-4.7 (m, 2 H), 5.9- |
| | | | | 5.9 (m, 2 H), 6.1-6.2 (m, 1 H), 6.3-6.4 (m, 1 H), 6.4-6.4 |
| | | | | (m, 1 H), 6.7-6.7 (m, 1 H), 6.8-6.8 (m, 1 H), 6.9-7.0 (m, 1 |
| | | | | H), 7.0-7.1 (m, 1 H), 7.1-7.2 (m, 1 H), 7.3-7.4 (m, 1 H), 7.4- |
| | | | | 7.4 (m, 1 H), 7.7-7.7 (m, 1 H), 8.1-8.1 (m, 1 H), 12.1-12.2 |
| | | | | (m, 1 H) |
| I-326 ^{b} | Q | D | 665.3 | 12.19-12.07 (m, 1H), 8.14-8.08 (m, 1H), 7.81-7.76 (m, 1H), |
| | | | | 7.72-7.64 (m, 1H), 7.41-7.33 (m, 2H), 7.29-7.24 (m, 1H), |
| | | | | 7.17-7.13 (m, 1H), 7.07-7.03 (m, 1H), 6.98-6.89 (m, 2H), |
| | | | | 6.45-6.41 (m, 1H), 6.17-6.09 (m, 1H), 4.65-4.59 (m, 2H), |
| | | | | 4.38-4.30 (m, 2H), 3.80 (s, 7H), 3.74-3.71 (m, 3H), 3.65 (br |
| | | | | t, J = 5.6 Hz, 1H), 3.60 (br t, J = 5.6 Hz, 1H), 3.42-3.33 (m, |
| | | | | 4H), 3.12-3.07 (m, 2H), 2.37-2.33 (m, 1H), 2.27 (br s, 1H) |
| I-327 | R | D | 663.4 | 12.02 (s, 1H), 8.17 (s, 2H), 7.73 (s, 1H), 7.61 (dd, J = 1.6, 7.2 |
| | | | | Hz, 1H), 7.45-7.33 (m, 2H), 7.19-7.12 (m, 1H), 7.08-7.01 |
| | | | | (m, 2H), 6.98-6.89 (m, 1H), 6.47-6.39 (m, 1H), 6.20 (s, 1H), |
| | | | | 4.68-4.59 (m, 2H), 4.41-4.28 (m, 2H), 3.85-3.79 (m, 4H), |
| | | | | 3.76-3.70 (m, 3H), 3.67-3.57 (m, 2H), 3.11-3.02 (m, 6H), |
| | | | | 2.71-2.59 (m, 3H), 2.36-2.26 (m, 2H), 1.23-1.17 (m, 3H) |
| I-328 | S | D | 660.1 | 12.69-11.69 (m, 1H), 8.47-8.36 (m, 1H), 8.15-8.07 (m, 2H), |
| | | | | 7.72-7.63 (m, 1H), 7.43-7.34 (m, 2H), 7.15 (br d, J = 8.0 Hz, |
| | | | | 1H), 7.07-7.02 (m, 1H), 6.98-6.89 (m, 2H), 6.48 (d, J = 2.8 |
| | | | | Hz, 1H), 6.16-6.08 (m, 1H), 4.65-4.60 (m, 2H), 4.38-4.31 |
| | | | | (m, 2H), 3.89-3.81 (m, 4H), 3.73 (br d, J = 3.6 Hz, 3H), 3.69 |
| | | | | (br s, 4H), 3.64-3.57 (m, 2H), 3.12-3.07 (m, 2H), 2.35-2.26 |
| | | | | (m, 2H) |
| I-329 | T | D | 703.3 | 12.16-12.10 (m, 1H), 8.54 (d, J = 4.4 Hz, 1H), 8.14-8.07 (m, |
| | | | | 2H), 7.71-7.65 (m, 1H), 7.42-7.32 (m, 2H), 7.27-7.22 (m, |
| | | | | 1H), 7.15 (d, J = 8.0 Hz, 1H), 7.05 (t, J = 7.6 Hz, 1H), 6.97- |
| | | | | 6.88 (m, 1H), 6.45 (s, 1H), 6.16-6.10 (m, 1H), 4.66-4.58 (m, |
| | | | | 2H), 4.39-4.30 (m, 2H), 3.81 (s, 4H), 3.73 (s, 3H), 3.67-3.57 |
| | | | | (m, 2H), 3.22 (s, 4H), 3.12-3.06 (m, 2H), 2.36-2.25 (m, 2H) |
| I-382 | CL | D | 664.5 | 12.19-12.07 (m, 1H), 8.16-8.01 (m, 2H), 7.73-7.63 (m, 1H), |
| | | | | 7.44-7.32 (m, 3H), 7.24 (s, 1H), 7.15 (br d, J = 8.0 Hz, 1H), |
| | | | | 7.09-7.01 (m, 1H), 6.99-6.89 (m, 1H), 6.39-6.35 (m, 1H), |
| | | | | 6.17-6.09 (m, 1H), 4.66-4.59 (m, 2H), 4.56-4.28 (m, 4H), |
| | | | | 3.88-3.79 (m, 3H), 3.76-3.68 (m, 3H), 3.67-3.56 (m, 2H), |
| | | | | 3.43-3.35 (m, 2H), 3.13-3.06 (m, 2H), 2.38-2.27 (m, 2H), |
| | | | | 1.84-1.68 (m, 4H), 0.98-0.89 (m, 1H) |
| I-330 ^{b} | U | D | 709.4 | 12.24-11.98 (m, 1H), 8.15-8.07 (m, 1H), 7.79 (dd, J = 1.2, |
| | | | | 4.8 Hz, 1H), 7.73-7.63 (m, 1H), 7.42-7.36 (m, 1H), 7.35 (dd, |
| | | | | J = 1.6, 7.4 Hz, 1H), 7.26 (dd, J = 1.2, 8.0 Hz, 1H), 7.15 (br d, |
| | | | | J = 8.4 Hz, 1H), 7.04 (t, J = 7.2 Hz, 1H), 6.97-6.86 (m, 2H), |
| | | | | 6.45-6.40 (m, 1H), 6.18-6.09 (m, 1H), 4.65-4.59 (m, 2H), |
| | | | | 4.38-4.30 (m, 2H), 4.13-4.09 (m, 2H), 3.79 (br s, 4H), 3.74- |
| | | | | 3.72 (m, 3H), 3.70-3.67 (m, 2H), 3.66-3.58 (m, 2H), 3.38 (br |
| | | | | s, 4H), 3.30 (s, 3H), 3.09 (q, J = 6.8 Hz, 2H), 2.37-2.26 (m, 2H) |
| I-332 | F | D | 693.6 | 12.16-12.06 (m, 1H), 8.15-8.07 (m, 1H), 7.80-7.76 (m, 1H), |
| | | | | 7.71-7.65 (m, 1H), 7.42-7.33 (m, 2H), 7.25 (d, J = 7.2 Hz, |
| | | | | 1H), 7.17-7.13 (m, 1H), 7.05 (t, J = 7.2 Hz, 1H), 6.98-6.94 |
| | | | | (m, 1H), 6.92-6.85 (m, 2H), 6.43 (br s, 1H), 6.17-6.10 (m, |
| | | | | 1H), 4.66-4.59 (m, 3H), 4.39-4.30 (m, 2H), 3.79 (br s, 4H), |
| | | | | 3.74-3.72 (m, 3H), 3.67-3.58 (m, 2H), 3.35 (br d, J = 2.4 Hz, |
| | | | | 4H), 3.09 (q, J = 6.4 Hz, 2H), 2.36-2.27 (m, 2H), 1.27 (d, J = |
| | | | | 6.0 Hz, 6H) |
| I-333 | H | D | 677.1 | 12.29-11.93 (m, 1H), 8.15-8.08 (m, 2H), 7.70-7.66 (m, 2H), |
| | | | | 7.42-7.33 (m, 2H), 7.15 (d, J = 8.4 Hz, 1H), 7.09-7.02 (m, |
| | | | | 2H), 6.97-6.89 (m, 1H), 6.45-6.42 (m, 1H), 6.16-6.10 (m, |
| | | | | 1H), 4.65-4.59 (m, 2H), 4.39-4.30 (m, 2H), 3.83 (s, 4H), |
| | | | | 3.73-3.71 (m, 3H), 3.65 (t, J = 5.6 Hz, 1H), 3.60 (t, J = 5.6 |
| | | | | Hz, 1H), 3.25 (d, J = 7.2 Hz, 1H), 3.09 (q, J = 7.2 Hz, 2H), |
| | | | | 3.01 (s, 4H), 2.37-2.26 (m, 2H), 1.18 (d, J = 6.8 Hz, 6H) |
| I-334 ^{b} | I | D | 727.4 | 2.2-2.4 (m, 2 H), 3.0-3.1 (m, 2 H), 3.4-3.4 (m, 4 H), 3.6- |
| | | | | 3.7 (m, 6 H), 3.7-3.7 (m, 3 H), 4.3-4.4 (m, 2 H), 4.6-4.7 |
| | | | | (m, 2 H), 6.1-6.2 (m, 1 H), 6.4-6.4 (m, 1 H), 6.9-7.0 (m, 4 |
| | | | | H), 7.0-7.1 (m, 1 H), 7.1-7.1 (m, 2 H), 7.3-7.3 (m, 1 H), |
| | | | | 7.3-7.4 (m, 4 H), 7.6-7.7 (m, 1 H), 8.0-8.1 (m, 2 H), 11.8- |
| | | | | 12.3 (m, 1 H) |
| I-335 | J | D | 679 | 1.3 (t, J = 6.4 Hz, 3 H), 2.3-2.4 (m, 2 H), 3.1 (br s, 2 H), 3.3- |
| | | | | 3.4 (m, 4 H), 3.6-3.6 (m, 1 H), 3.6-3.7 (m, 1 H), 3.7-3.7 (m, |
| | | | | 3 H), 3.8-3.8 (m, 4 H), 4.0-4.1 (m, 2 H), 4.3-4.4 (m, 2 H), |
| | | | | 4.6-4.7 (m, 2 H), 6.1-6.2 (m, 1 H), 6.4-6.5 (m, 1 H), 6.9- |
| | | | | 7.0 (m, 2 H), 7.0-7.1 (m, 1 H), 7.1-7.2 (m, 1 H), 7.2-7.3 (m, |
| | | | | 1 H), 7.3-7.4 (m, 1 H), 7.4-7.4 (m, 1 H), 7.7-7.7 (m, 1 H), |
| | | | | 7.8-7.8 (m, 1 H), 8.1-8.1 (m, 1 H), 12.1-12.1 (m, 1 H) |
| I-336 | G | D | 691.2 | 12.16-12.08 (m, 1H), 8.14-8.08 (m, 1H), 7.81 (br d, J = 3.6 |
| | | | | Hz, 1H), 7.71-7.65 (m, 1H), 7.53 (br d, J = 7.6 Hz, 1H), 7.41- |
| | | | | 7.33 (m, 2H), 7.15 (br d, J = 8.4 Hz, 1H), 7.05 (br t, J = 7.6 Hz, |
| | | | | 1H), 6.97-6.89 (m, 2H), 6.42 (br s, 1H), 6.16-6.10 (m, 1H), |
| | | | | 4.65-4.59 (m, 2H), 4.38-4.30 (m, 2H), 3.88 (br s, 1H), 3.77 |
| | | | | (br s, 4H), 3.73 (br s, 3H), 3.66-3.58 (m, 2H), 3.30-3.28 (m, |
| | | | | 4H), 3.12-3.07 (m, 2H), 2.37-2.26 (m, 2H), 0.82-0.76 (m, |
| | | | | 2H), 0.70 (br s, 2H) |
| I-343 ^{b} | G | AA | 679.3 | 12.35-12.25 (m, 1H), 8.19-8.05 (m, 1H), 7.87-7.80 (m, |
| | | | | 1H), 7.74-7.67 (m, 1H), 7.63-7.58 (m, 1H), 7.56-7.52 (m, |
| | | | | 1H), 7.50-7.46 (m, 1H), 7.40-7.32 (m, 2H), 7.13-7.00 (m, |
| | | | | 1H), 6.98-6.92 (m, 1H), 6.63-6.51 (m, 1H), 6.22-6.13 (m, |
| | | | | 1H), 4.72-4.59 (m, 2H), 4.42-4.31 (m, 2H), 3.91-3.86 (m, |
| | | | | 1H), 3.80-3.74 (m, 4H), 3.69-3.60 (m, 2H), 3.29-3.22 (m, |
| | | | | 2H), 3.18-3.05 (m, 3H), 2.38-2.28 (m, 2H), 0.83-0.70 (m, 4H) |
| I-344 ^{b} | G | AB | 675.3 | 12.31-12.11 (m, 1H), 8.18-8.06 (m, 1H), 7.83-7.77 (m, |
| | | | | 1H), 7.72-7.64 (m, 1H), 7.58-7.47 (m, 1H), 7.37-7.26 (m, |
| | | | | 4H), 6.94-6.89 (m, 1H), 6.85-6.78 (m, 1H), 6.36-6.28 (m, |
| | | | | 1H), 6.22-6.08 (m, 1H), 4.66-4.57 (m, 2H), 4.42-4.29 (m, |
| | | | | 2H), 3.91-3.83 (m, 1H), 3.78-3.68 (m, 4H), 3.66-3.57 (m, |
| | | | | 2H), 3.29-3.23 (m, 4H), 3.12-3.07 (m, 2H), 2.36-2.25 (m, |
| | | | | 2H), 2.15 (s, 3H), 0.85-0.65 (m, 4H) |
| I-345 | G | AG | 690.4 | 12.11 (d, J = 8.4 Hz, 1H), 7.83-7.78 (m, 1H), 7.75-7.68 (m, |
| | | | | 1H), 7.56 (d, J = 8.0 Hz, 1H), 7.45-7.31 (m, 3H), 7.15 (d, J = |
| | | | | 8.0 Hz, 1H), 7.05 (t, J = 7.2 Hz, 1H), 6.98-6.87 (m, 2H), 6.45- |
| | | | | 6.40 (m, 1H), 6.22-6.16 (m, 1H), 6.15-6.06 (m, 1H), 4.41- |
| | | | | 4.32 (m, 3H), 4.28 (s, 1H), 3.93-3.85 (m, 1H), 3.83-3.68 (m, |
| | | | | 8H), 3.68-3.61 (m, 1H), 3.60-3.53 (m, 2H), 2.97 (q, J = 7.2 |
| | | | | Hz, 2H), 2.37-2.22 (m, 2H), 0.85-0.75 (m, 2H), 0.74-0.63 |
| | | | | (m, 2H) |
| I-347 | AL | D | 665.4 | 12.16-12.11 (m, 1H), 8.14-8.08 (m, 1H), 7.77 (dd, J = 1.6, |
| | | | | 4.8 Hz, 1H), 7.74-7.63 (m, 1H), 7.39 (t, J = 7.6 Hz, 1H), 7.35 |
| | | | | (dd, J = 1.6, 7.2 Hz, 1H), 7.21 (dd, J = 1.2, 7.6 Hz, 1H), 7.15 |
| | | | | (d, J = 8.4 Hz, 1H), 7.05 (t, J = 7.2 Hz, 1H), 6.97-6.89 (m, |
| | | | | 2H), 6.44 (s, 1H), 6.13 (d, J = 11.2 Hz, 1H), 4.65-4.59 (m, |
| | | | | 2H), 4.38-4.30 (m, 2H), 3.88 (s, 3H), 3.82 (s, 4H), 3.74-3.72 |
| | | | | (m, 3H), 3.65 (t, J = 5.6 Hz, 1H), 3.60 (t, J = 5.6 Hz, 1H), 3.12- |
| | | | | 3.08 (m, 2H), 3.07 (s, 4H), 2.36-2.26 (m, 2H) |
| I-349 | AU | D | 664.4 | 2.35-2.50 (m, 2 H) 3.09 (br s, 4 H) 3.20 (br d, J = 6.8 Hz, 2 H) |
| | | | | 3.65-3.82 (m, 2 H) 3.84 (d, J = 3.6 Hz, 3 H) 3.87-3.97 (m, 7 |
| | | | | H) 4.36-4.52 (m, 2 H) 4.63-4.80 (m, 2 H) 6.06-6.29 (m, 1 |
| | | | | H) 6.55 (br s, 1 H) 6.97-7.10 (m, 5 H) 7.16 (t, J = 7.2 Hz, 1 H) |
| | | | | 7.27 (br d, J = 8.4 Hz, 1 H) 7.44-7.48 (m, 1 H) 7.48-7.57 (m, |
| | | | | 1 H) 7.68-7.84 (m, 1 H) 8.18-8.25 (m, 1 H) 12.24 (br d, |
| | | | | J = 6.8 Hz, 1 H) |
| I-350 | AV | D | 694.3 | 12.13 (d, J = 7.2 Hz, 1H), 8.14-8.08 (m, 1H), 7.75-7.60 (m, |
| | | | | 1H), 7.42-7.33 (m, 2H), 7.15 (d, J = 8.0 Hz, 1H), 7.07-7.00 |
| | | | | (m, 2H), 6.97-6.89 (m, 1H), 6.61 (d, J = 8.4 Hz, 2H), 6.40 (s, |
| | | | | 1H), 6.16-6.09 (m, 1H), 4.65-4.59 (m, 2H), 4.38-4.29 (m, |
| | | | | 2H), 3.73 (s, 6H), 3.72 (d, J = 3.2 Hz, 4H), 3.65 (t, J = 5.6 Hz, |
| | | | | 1H), 3.60 (t, J = 5.6 Hz, 1H), 3.33 (s, 3H), 3.09 (q, J = 6.8 Hz, |
| | | | | 2H), 3.02 (s, 4H), 2.36-2.24 (m, 2H) |
| I-351 | AW | D | 679.4 | 12.14-12.09 (m, 1H), 8.13-8.08 (m, 1H), 7.72-7.65 (m, 1H), |
| | | | | 7.39 (t, J = 8.0 Hz, 1H), 7.35 (dd, J = 1.2, 7.6 Hz, 1H), 7.15 (d, |
| | | | | J = 8.0 Hz, 2H), 7.07-7.03 (m, 1H), 6.97-6.89 (m, 1H), 6.74 |
| | | | | (d, J = 8.0 Hz, 1H), 6.43 (s, 1H), 6.13 (d, J = 11.2 Hz, 1H), |
| | | | | 4.65-4.59 (m, 2H), 4.38-4.30 (m, 2H), 3.80-3.75 (m, 8H), |
| | | | | 3.74-3.72 (m, 3H), 3.65 (t, J = 5.6 Hz, 1H), 3.59 (t, J = 5.6 |
| | | | | Hz, 2H), 3.32 (s, 2H), 3.12-3.10 (m, 1H), 3.09-3.06 (m, 1H), |
| | | | | 2.35 (s, 2H), 2.27 (s, 3H) |
| I-352 | AX | D | 695.3 | 12.15-12.09 (m, 1H), 8.14-8.10 (m, 1H), 7.72-7.66 (m, 1H), |
| | | | | 7.54 (d, J = 2.4 Hz, 1H), 7.43-7.38 (m, 1H), 7.35 (dd, J = 1.6, |
| | | | | 7.2 Hz, 1H), 7.16 (br d, J = 8.4 Hz, 1H), 7.06 (t, J = 7.2 Hz, |
| | | | | 1H), 6.98 (d, J = 2.4 Hz, 1H), 6.97-6.89 (m, 1H), 6.43 (br s, |
| | | | | 1H), 6.18-6.10 (m, 1H), 4.67-4.59 (m, 2H), 4.41-4.30 (m, |
| | | | | 2H), 3.80 (d, J = 12.4 Hz, 10H), 3.75-3.72 (m, 3H), 3.66 (br t, |
| | | | | J = 5.6 Hz, 1H), 3.60 (br t, J = 5.6 Hz, 1H), 3.18 (br s, 4H), |
| | | | | 3.10 (q, J = 6.8 Hz, 2H), 2.35 (br d, J = 2.4 Hz, 1H), 2.28 (br s, 1H) |
| I-353 | AY | D | 694.4 | 12.12 (br d, J = 6.4 Hz, 1H), 8.22-8.02 (m, 1H), 7.77-7.58 |
| | | | | (m, 1H), 7.47-7.26 (m, 2H), 7.24-7.12 (m, 2H), 7.06 (t, J = |
| | | | | 7.2 Hz, 1H), 7.00-6.86 (m, 1H), 6.59 (d, J = 2.4 Hz, 1H), 6.41 |
| | | | | (br s, 1H), 6.20-6.06 (m, 1H), 5.39 (br d, J = 5.2 Hz, 1H), |
| | | | | 4.70-4.57 (m, 2H), 4.44-4.27 (m, 2H), 3.86-3.75 (m, 7H), |
| | | | | 3.74-3.71 (m, 3H), 3.68-3.58 (m, 2H), 3.13-3.04 (m, 6H), |
| | | | | 2.67 (d, J = 4.8 Hz, 3H), 2.55-2.46 (m, 12H), 2.40-2.22 (m, 2H) |
| I-354 | AZ | D | 705.4 | 12.22-12.05 (m, 1H), 8.12 (d, J = 10.8 Hz, 1H), 7.75-7.56 |
| | | | | (m, 2H), 7.44-7.33 (m, 2H), 7.22-7.12 (m, 1H), 7.06 (t, J = |
| | | | | 7.6 Hz, 1H), 6.98-6.87 (m, 2H), 6.43 (br d, J = 2.0 Hz, 1H), |
| | | | | 6.18-6.09 (m, 1H), 4.69-4.59 (m, 2H), 4.43-4.30 (m, 2H), |
| | | | | 3.80 (s, 6H), 3.73 (br d, J = 3.6 Hz, 3H), 3.68-3.60 (m, 2H), |
| | | | | 3.25 (br s, 4H), 3.14-3.07 (m, 2H), 2.39-2.21 (m, 3H), 1.91- |
| | | | | 1.81 (m, 1H), 0.95-0.85 (m, 2H), 0.71-0.64 (m, 2H) |
| I-364 | G | AH | 693.3 | 12.07-11.95 (m, 1H), 8.12 (d, J = 11.2 Hz, 1H), 7.86-7.76 |
| | | | | (m, 1H), 7.72 (d, J = 10.0 Hz, 1H), 7.57-7.49 (m, 1H), 7.43- |
| | | | | 7.31 (m, 2H), 7.16 (d, J = 8.0 Hz, 1H), 7.04 (t, J = 7.2 Hz, 1H), |
| | | | | 6.98-6.91 (m, 2H), 6.40 (s, 1H), 4.65-4.57 (m, 2H), 4.48 (t, J = |
| | | | | 10.0 Hz, 1H), 3.94-3.85 (m, 2H), 3.76 (s, 4H), 3.72 (s, 3H), |
| | | | | 3.28-3.10 (m, 4H), 3.10-2.92 (m, 4H), 2.80-2.61 (m, 1H), |
| | | | | 1.99-1.84 (m, 2H), 1.76 (d, J = 13.2 Hz, 1H), 1.53-1.24 (m, |
| | | | | 1H), 0.83-0.76 (m, 2H), 0.74-0.67 (m, 2H) |
| I-365 | AM | D | 665.3 | 12.18-12.11 (m, 1H), 8.16-8.09 (m, 2H), 8.04 (d, J = 5.2 Hz, |
| | | | | 1H), 7.72-7.66 (m, 1H), 7.43-7.37 (m, 1H), 7.35 (dd, J = 1.6, |
| | | | | 7.6 Hz, 1H), 7.16 (br d, J = 8.0 Hz, 1H), 7.08-7.02 (m, 1H), |
| | | | | 6.99-6.90 (m, 1H), 6.84 (d, J = 5.2 Hz, 1H), 6.45 (br d, J = 2.4 |
| | | | | Hz, 1H), 6.18-6.08 (m, 1H), 4.68-4.59 (m, 2H), 4.42-4.29 |
| | | | | (m, 2H), 3.87 (s, 3H), 3.82 (br s, 4H), 3.76-3.73 (m, 3H), 3.66 |
| | | | | (br t, J = 5.6 Hz, 1H), 3.60 (br t, J = 5.6 Hz, 1H), 3.20 (br s, |
| | | | | 4H), 3.10 (q, J = 7.2 Hz, 2H), 2.38-2.25 (m, 2H) |
| I-366 | Q | BJ | 666.3 | 12.28-12.19 (m, 1H), 8.50 (s, 1H), 8.33-8.29 (m, 1H), 8.11 |
| | | | | (br d, J = 11.2 Hz, 1H), 7.84-7.75 (m, 1H), 7.71-7.65 (m, |
| | | | | 1H), 7.43-7.38 (m, 1H), 7.29-7.23 (m, 1H), 7.09-6.99 (m, |
| | | | | 1H), 6.92 (dd, J = 4.8, 8.0 Hz, 1H), 6.55-6.50 (m, 1H), 6.18- |
| | | | | 6.10 (m, 1H), 4.65-4.59 (m, 2H), 4.38-4.30 (m, 2H), 3.88- |
| | | | | 3.73 (m, 12H), 3.67-3.54 (m, 3H), 3.13-3.04 (m, 3H), 2.37- |
| | | | | 2.28 (m, 2H) |
| I-367 | BK | D | 682.2 | 12.13 (d, J = 7.2 Hz, 1H), 8.17-8.04 (m, 1H), 7.74-7.60 (m, |
| | | | | 1H), 7.38 (d, J = 7.6 Hz, 1H), 7.34 (dd, J = 1.6, 7.6 Hz, 1H), |
| | | | | 7.15 (d, J = 8.4 Hz, 1H), 7.10-7.02 (m, 2H), 6.97-6.89 (m, |
| | | | | 1H), 6.82 (d, J = 8.4 Hz, 1H), 6.76 (dd, J = 8.8, 10.4 Hz, 1H), |
| | | | | 6.41 (s, 1H), 6.19-6.07 (m, 1H), 4.66-4.59 (m, 2H), 4.39- |
| | | | | 4.30 (m, 2H), 3.79 (s, 3H), 3.75 (s, 3H), 3.73-3.71 (m, 3H), |
| | | | | 3.65 (t, J = 5.6Hz, 1H), 3.60 (t, J = 5.6 Hz, 1H), 3.12-3.05 (m, |
| | | | | 6H), 2.39-2.22 (m, 3H) |
| I-368 | BL | BM | 666.5 | 12.23 (s, 1H), 8.51 (d, J = 5.6 Hz, 1H), 8.42 (s, 1H), 8.15- |
| | | | | 8.07 (m, 1H), 7.80 (dd, J = 1.2, 4.8 Hz, 1H), 7.73-7.65 (m, |
| | | | | 1H), 7.31-7.18 (m, 2H), 7.05-6.89 (m, 2H), 6.49 (d, J = 1.6 |
| | | | | Hz, 1H), 6.21-6.12 (m, 1H), 4.68-4.58 (m, 2H), 4.41-4.29 |
| | | | | (m, 2H), 3.92-3.75 (m, 12H), 3.74-3.50 (m, 3H), 3.24-3.04 |
| | | | | (m, 3H), 2.38-2.25 (m, 2H) |
| I-369 | BN | D | 699.3 | 12.12 (br d, J = 6.4 Hz, 1H), 8.13-8.09 (m, 1H), 7.82 (d, J = |
| | | | | 2.0 Hz, 1H), 7.70-7.66 (m, 1H), 7.41-7.37 (m, 2H), 7.35 (br |
| | | | | d, J = 7.6 Hz, 1H), 7.15 (br d, J = 8.4 Hz, 1H), 7.05 (t, J = 7.2 |
| | | | | Hz, 1H), 6.97-6.89 (m, 1H), 6.43 (br s, 1H), 6.15-6.10 (m, |
| | | | | 1H), 4.62 (br d, J = 8.4 Hz, 2H), 4.38-4.30 (m, 2H), 3.84 (s, |
| | | | | 3H), 3.78 (br s, 6H), 3.72 (d, J = 3.6 Hz, 4H), 3.65 (br t, J = 5.6 |
| | | | | Hz, 1H), 3.61-3.58 (m, 1H), 3.12-3.07 (m, 2H), 2.37-2.31 |
| | | | | (m, 2H), 2.27 (br s, 1H) |
| I-370 | BO | D | 683.4 | 12.17-12.04 (m, 1H), 8.14-8.07 (m, 1H), 7.78 (d, J = 2.4 Hz, |
| | | | | 1H), 7.71-7.65 (m, 1H), 7.42-7.32 (m, 3H), 7.15 (d, J = 8.4 |
| | | | | Hz, 1H), 7.08-7.02 (m, 1H), 6.98-6.87 (m, 1H), 6.45-6.40 |
| | | | | (m, 1H), 6.17-6.08 (m, 1H), 4.67-4.57 (m, 2H), 4.41-4.27 |
| | | | | (m, 2H), 3.83 (s, 3H), 3.79 (s, 4H), 3.74-3.70 (m, 3H), 3.67- |
| | | | | 3.56 (m, 2H), 3.25 (s, 4H), 3.09 (q, J = 7.2 Hz, 2H), 2.38-2.23 |
| | | | | (m, 2H) |
| I-387 | CR | D | 699.2 | 12.14 (br s, 1H), 8.16-8.06 (m, 1H), 7.92 (d, J = 5.2 Hz, 1H), |
| | | | | 7.76-7.63 (m, 1H), 7.45-7.33 (m, 2H), 7.15 (br d, J = 8.4 Hz, |
| | | | | 1H), 7.08-7.01 (m, 2H), 6.99-6.89 (m, 1H), 6.45 (br s, 1H), |
| | | | | 6.18-6.08 (m, 1H), 4.66-4.59 (m, 2H), 4.39-4.28 (m, 2H), |
| | | | | 3.83 (br s, 4H), 3.76-3.72 (m, 6H), 3.66-3.58 (m, 2H), 3.46 |
| | | | | (br s, 4H), 3.09 (q, J = 6.4 Hz, 2H), 2.37-2.27 (m, 2H) |
| I-388 | CS | D | 683.3 | 12.13 (d, J = 7.2 Hz, 1H), 8.14-8.07 (m, 1H), 7.93 (dd, J = |
| | | | | 5.6, 8.0 Hz, 1H), 7.73-7.63 (m, 1H), 7.43-7.32 (m, 2H), 7.15 |
| | | | | (d, J = 8.4 Hz, 1H), 7.05 (t, J = 7.2 Hz, 1H), 6.97-6.87 (m, |
| | | | | 2H), 6.44 (s, 1H), 6.18-6.08 (m, 1H), 4.67-4.57 (m, 2H), |
| | | | | 4.39-4.30 (m, 2H), 3.80 (s, 7H), 3.73 (d, J = 3.6 Hz, 3H), 3.67- |
| | | | | 3.58 (m, 2H), 3.46 (s, 4H), 3.09 (q, J = 6.8 Hz, 2H), 2.37- |
| | | | | 2.25 (m, 2H) |
| I-389 | Q | AA | 653.2 | 12.30 (d, J = 5.2 Hz, 1H), 8.21-8.03 (m, 1H), 7.85-7.77 (m, |
| | | | | 1H), 7.74-7.65 (m, 1H), 7.63-7.57 (m, 1H), 7.51-7.43 (m, |
| | | | | 1H), 7.39 (s, 2H), 7.27 (d, J = 8.0 Hz, 1H), 7.15-6.99 (m, 1H), |
| | | | | 6.94-6.86 (m, 1H), 6.63-6.48 (m, 1H), 6.24-6.05 (m, 1H), |
| | | | | 4.67-4.59 (m, 2H), 4.41-4.33 (m, 2H), 3.81 (s, 3H), 3.80- |
| | | | | 3.70 (m, 4H), 3.68-3.59 (m, 2H), 3.37-3.34 (m, 2H), 3.10 (q, |
| | | | | J = 7.2 Hz, 2H), 2.50-2.45 (m, 2H), 2.38-2.27 (m, 2H) |
| I-391 | CU | D | 683.4 | 12.21-11.99 (m, 1H), 8.13-8.08 (m, 1H), 7.98 (dd, J = 1.2, |
| | | | | 4.8 Hz, 1H), 7.73-7.64 (m, 1H), 7.45 (d, J = 8.4 Hz, 1H), 7.42- |
| | | | | 7.37 (m, 1H), 7.35 (dd, J = 1.6, 7.6 Hz, 1H), 7.15 (br d, J = |
| | | | | 8.0 Hz, 1H), 7.05 (t, J = 7.2 Hz, 1H), 6.97-6.89 (m, 2H), 6.45- |
| | | | | 6.42 (m, 1H), 6.17-6.10 (m, 1H), 5.95 (s, 1H), 5.81 (s, 1H), |
| | | | | 4.65-4.59 (m, 2H), 4.38-4.30 (m, 2H), 3.80 (br s, 4H), 3.72- |
| | | | | 3.72 (m, 1H), 3.74-3.71 (m, 3H), 3.67-3.58 (m, 2H), 3.35 |
| | | | | (br d, J = 3.2 Hz, 4H), 3.09 (q, J = 6.8 Hz, 2H), 2.37-2.26 (m, 2H) |
| I-392 | CV | D | 664.6 | 12.13 (d, J = 7.6 Hz, 1H), 8.13-8.09 (m, 1H), 7.76-7.62 (m, |
| | | | | 1H), 7.42-7.37 (m, 1H), 7.34 (dd, J = 1.6, 7.6 Hz, 1H), 7.17- |
| | | | | 7.10 (m, 2H), 7.05 (t, J = 7.2 Hz, 1H), 6.97-6.89 (m, 1H), |
| | | | | 6.53 (dd, J = 1.6, 8.4 Hz, 1H), 6.47 (t, J = 2.0 Hz, 1H), 6.44 (s, |
| | | | | 1H), 6.39 (dd, J = 2.0, 8.0 Hz, 1H), 6.16-6.10 (m, 1H), 4.65- |
| | | | | 4.59 (m, 2H), 4.39-4.31 (m, 2H), 3.80 (s, 4H), 3.74 (d, J = 3.2 |
| | | | | Hz, 3H), 3.71 (s, 3H), 3.65 (t, J = 5.6 Hz, 1H), 3.60 (t, J = 5.6 |
| | | | | Hz, 1H), 3.19 (s, 4H), 3.12-3.07 (m, 2H), 2.36-2.26 (m, 2H) |
| I-393 | CW | D | 659.2 | 12.18-12.13 (m, 1H), 8.13-8.09 (m, 1H), 7.73 (dd, J = 1.6, |
| | | | | 7.6 Hz, 1H), 7.71-7.64 (m, 1H), 7.64-7.59 (m, 1H), 7.42- |
| | | | | 7.37 (m, 1H), 7.35 (dd, J = 1.6, 7.6 Hz, 1H), 7.19 (d, J = 8.4 |
| | | | | Hz, 1H), 7.16-7.11 (m, 2H), 7.05 (dt, J = 0.8, 7.2 Hz, 1H), |
| | | | | 6.98-6.90 (m, 1H), 6.47 (s, 1H), 6.16-6.10 (m, 1H), 4.65- |
| | | | | 4.59 (m, 2H), 4.38-4.31 (m, 2H), 3.87 (s, 4H), 3.74-3.72 (m, |
| | | | | 3H), 3.65 (t, J = 6.0 Hz, 1H), 3.60 (t, J = 5.6 Hz, 1H), 3.19 (s, |
| | | | | 4H), 3.12-3.07 (m, 2H), 2.36-2.25 (m, 2H) |
| I-407 | AU | BJ | 665.3 | 12.25 (s, 1H), 8.50 (s, 1H), 8.30 (d, J = 4.8 Hz, 1H), 8.14- |
| | | | | 8.06 (m, 1H), 7.73-7.62 (m, 1H), 7.43-7.38 (m, 1H), 7.01 |
| | | | | (d, J = 6.4 Hz, 1H), 6.99-6.93 (m, 2H), 6.92-6.85 (m, 2H), |
| | | | | 6.53 (d, J = 2.8 Hz, 1H), 6.21-6.07 (m, 1H), 4.62 (q, J = 7.2 |
| | | | | Hz, 2H), 4.39-4.28 (m, 2H), 3.86 (d, J = 3.2 Hz, 3H), 3.84- |
| | | | | 3.77 (m, 7H), 3.67-3.58 (m, 2H), 3.13-3.06 (m, 2H), 2.99 (s, |
| | | | | 4H), 2.37-2.26 (m, 2H) |
| I-408 | Q | DN | 585.2 | 12.24 (br s, 1H), 8.51 (s, 1H), 8.30 (dd, J = 2.4, 4.8 Hz, 1H), |
| | | | | 7.79 (dd, J = 1.2, 4.8 Hz, 1H), 7.41 (d, J = 4.8 Hz, 1H), 7.27 (d, |
| | | | | J = 7.2 Hz, 1H), 7.10-6.96 (m, 1H), 6.92 (dd, J = 4.8, 7.6 Hz, |
| | | | | 1H), 6.53 (d, J = 3.2 Hz, 1H), 6.22-6.07 (m, 1H), 4.33 (br d, J = |
| | | | | 8.8 Hz, 2H), 3.86 (s, 3H), 3.80 (s, 7H), 3.66-3.57 (m, 2H), |
| | | | | 3.34 (br s, 4H), 2.40-2.25 (m, 2H), 2.08 (d, J = 7.2 Hz, 3H) |
| I-413 | DY | D | 731.4 | 12.11 (br d, J = 7.2 Hz, 1H), 8.16-8.06 (m, 1H), 7.81-7.60 |
| | | | | (m, 2H), 7.44-7.32 (m, 2H), 7.22-7.13 (m, 2H), 7.05 (t, J = |
| | | | | 7.2 Hz, 1H), 6.99-6.87 (m, 1H), 6.42 (br s, 1H), 6.19-6.09 |
| | | | | (m, 1H), 4.71-4.58 (m, 2H), 4.44-4.29 (m, 2H), 3.91 (tt, J = |
| | | | | 3.2, 6.0 Hz, 1H), 3.80-3.70 (m, 7H), 3.69-3.55 (m, 2H), 3.21 |
| | | | | (br s, 4H), 3.10 (q, J = 6.8 Hz, 2H), 2.41-2.25 (m, 2H), 1.94- |
| | | | | 1.83 (m, 1H), 0.96-0.89 (m, 2H), 0.84-0.76 (m, 2H), 0.73- |
| | | | | 0.63 (m, 4H) |
| I-414 | EB | D | 756.2 | 12.11-12.01 (m, 1H), 8.14-8.06 (m, 1H), 7.73-7.60 (m, 1H), |
| | | | | 7.42-7.37 (m, 1H), 7.33-7.27 (m, 3H), 7.13 (br t, J = 8.4 Hz, |
| | | | | 2H), 7.07-6.99 (m, 2H), 6.90-6.81 (m, 4H), 6.67-6.62 (m, |
| | | | | 1H), 6.28 (br s, 1H), 6.16-6.09 (m, 1H), 4.66-4.60 (m, 2H), |
| | | | | 4.39-4.30 (m, 2H), 3.80 (s, 3H), 3.69-3.63 (m, 4H), 3.59 (br |
| | | | | t, J = 5.6 Hz, 1H), 3.42 (br s, 4H), 3.11-3.06 (m, 2H), 2.93 (br |
| | | | | s, 4H), 2.35-2.25 (m, 2H) |
| I-415 | EC | D | 679.3 | 12.12 (d, J = 7.2 Hz, 1H), 8.13-8.08 (m, 1H), 7.82 (d, J = 4.8 |
| | | | | Hz, 1H), 7.75-7.59 (m, 1H), 7.42-7.34 (m, 2H), 7.15 (d, J = |
| | | | | 8.0 Hz, 1H), 7.05 (t, J = 7.6 Hz, 1H), 6.98-6.89 (m, 1H), 6.79 |
| | | | | (d, J = 4.8 Hz, 1H), 6.44 (s, 1H), 6.16-6.10 (m, 1H), 4.65- |
| | | | | 4.59 (m, 2H), 4.38-4.31 (m, 2H), 3.83 (s, 4H), 3.73 (d, J = 3.2 |
| | | | | Hz, 3H), 3.70 (s, 3H), 3.65 (t, J = 5.6 Hz, 1H), 3.60 (t, J = 5.6 |
| | | | | Hz, 1H), 3.36 (s, 4H), 3.09 (q, J = 6.8 Hz, 2H), 2.36-2.26 (m, |
| | | | | 2H), 2.19 (s, 3H) |
| I-416 | ED | D | 708.2 | 12.12 (br d, J = 6.4 Hz, 1H), 8.15-8.07 (m, 1H), 7.77-7.61 |
| | | | | (m, 1H), 7.45-7.29 (m, 3H), 7.16 (br d, J = 8.4 Hz, 1H), 7.06 |
| | | | | (t, J = 7.2 Hz, 1H), 6.99-6.89 (m, 1H), 6.78 (d, J = 2.4 Hz, |
| | | | | 1H), 6.42 (br s, 1H), 6.19-6.07 (m, 1H), 4.72-4.57 (m, 2H), |
| | | | | 4.46-4.27 (m, 2H), 3.86-3.72 (m, 10H), 3.69-3.57 (m, 2H), |
| | | | | 3.18-3.05 (m, 6H), 2.85 (s, 6H), 2.41-2.24 (m, 2H) |
| I-417 | EE | D | 686.2 | 12.09 (br d, J = 9.6 Hz, 1H), 8.18-8.10 (m, 1H), 8.04 (d, J = |
| | | | | 4.0 Hz, 1H), 7.80-7.55 (m, 2H), 7.51-7.27 (m, 5H), 7.19 (br |
| | | | | d, J = 7.6 Hz, 1H), 7.12-7.05 (m, 1H), 7.03-6.89 (m, 1H), |
| | | | | 6.77 (br s, 1H), 6.15 (br d, J = 11.2 Hz, 1H), 4.89 (br s, 2H), |
| | | | | 4.72 (s, 2H), 4.67-4.59 (m, 2H), 4.46-4.29 (m, 2H), 3.92 (s, |
| | | | | 3H), 3.78 (d, J = 3.2 Hz, 3H), 3.70-3.57 (m, 2H), 3.11 (q, J = |
| | | | | 6.4 Hz, 2H), 2.43-2.25 (m, 2H) |
| I-418 | EF | D | 652.4 | 12.18-12.08 (m, 1H), 8.13-8.09 (m, 1H), 7.70-7.66 (m, 1H), |
| | | | | 7.42-7.37 (m, 1H), 7.35 (br d, J = 6.4 Hz, 1H), 7.17-7.13 (m, |
| | | | | 2H), 7.11 (br d, J = 8.0 Hz, 1H), 7.07 (br d, J = 2.4 Hz, 1H), |
| | | | | 7.05-7.03 (m, 1H), 7.00 (br d, J = 5.6 Hz, 1H), 6.97-6.89 (m, |
| | | | | 1H), 6.44 (br s, 1H), 6.13 (br d, J = 9.2 Hz, 1H), 4.64-4.59 |
| | | | | (m, 2H), 4.38-4.31 (m, 2H), 3.84 (br s, 4H), 3.74-3.72 (m, |
| | | | | 3H), 3.65 (br t, J = 5.2 Hz, 1H), 3.61-3.58 (m, 1H), 3.12- |
| | | | | 3.08 (m, 2H), 3.05 (br s, 4H), 2.35 (br s, 1H), 2.27 (br d, J = |
| | | | | 1.6 Hz, 1H) |
| I-419 | EG | D | 634.3 | 12.14 (d, J = 7.2 Hz, 1H), 8.13-8.09 (m, 1H), 7.71-7.65 (m, |
| | | | | 1H), 7.42-7.37 (m, 1H), 7.35 (dd, J = 1.6, 7.6 Hz, 1H), 7.25- |
| | | | | 7.20 (m, 2H), 7.15 (d, J = 8.4 Hz, 1H), 7.05 (t, J = 7.2 Hz, 1H), |
| | | | | 6.97-6.90 (m, 3H), 6.81 (t, J = 7.2 Hz, 1H), 6.44 (s, 1H), 6.15- |
| | | | | 6.10 (m, 1H), 4.65-4.60 (m, 2H), 4.38-4.31 (m, 2H), 3.82 |
| | | | | (s, 4H), 3.74 (d, J = 3.2 Hz, 3H), 3.65 (t, J = 5.6 Hz, 1H), 3.60 |
| | | | | (t, J = 5.6 Hz, 1H), 3.19 (s, 4H), 3.12-3.07 (m, 2H), 2.36- |
| | | | | 2.26 (m, 2H) |
| I-420 ^{b} | EH | D | 718.2 | 12.16-12.11 (m, 1H), 8.14-8.08 (m, 1H), 7.75-7.61 (m, 1H), |
| | | | | 7.42-7.37 (m, 1H), 7.36-7.28 (m, 3H), 7.19-7.14 (m, 2H), |
| | | | | 7.13-7.08 (m, 1H), 7.07-7.02 (m, 1H), 6.97-6.89 (m, 1H), |
| | | | | 6.49-6.43 (m, 1H), 6.16-6.10 (m, 1H), 4.65-4.59 (m, 2H), |
| | | | | 4.39-4.30 (m, 2H), 3.81 (s, 4H), 3.74-3.71 (m, 3H), 3.67- |
| | | | | 3.58 (m, 2H), 3.09 (d, J = 7.2 Hz, 2H), 3.03 (s, 4H), 2.36-2.26 |
| | | | | (m, 2H) |
| I-421 | EI | D | 648.4 | 12.20-12.09 (m, 1H), 8.13-8.09 (m, 1H), 7.71-7.66 (m, 1H), |
| | | | | 7.42-7.37 (m, 1H), 7.35 (dd, J = 1.2, 7.2 Hz, 1H), 7.18-7.16 |
| | | | | (m, 1H), 7.14-7.12 (m, 1H), 7.07-7.02 (m, 2H), 7.00 (d, J = |
| | | | | 6.8 Hz, 1H), 6.98-6.95 (m, 1H), 6.90 (d, J = 6.0 Hz, 1H), 6.44 |
| | | | | (d, J = 2.8 Hz, 1H), 6.16-6.11 (m, 1H), 4.63 (br d, J = 7.2 Hz, |
| | | | | 2H), 4.38-4.32 (m, 2H), 3.83 (br s, 4H), 3.74-3.72 (m, 3H), |
| | | | | 3.66 (br t, J = 5.6 Hz, 1H), 3.60 (br t, J = 5.6 Hz, 1H), 3.10 (br |
| | | | | d, J = 7.2 Hz, 2H), 2.87 (br s, 4H), 2.38-2.31 (m, 2H), 2.28 (s, 3H) |
| I-422 | EJ | D | 663.3 | 12.10 (br d, J = 7.6 Hz, 1H), 8.17-8.03 (m, 1H), 7.75-7.62 |
| | | | | (m, 1H), 7.41-7.31 (m, 2H), 7.21-7.12 (m, 3H), 7.04 (t, J = |
| | | | | 7.6 Hz, 1H), 6.98-6.86 (m, 3H), 6.39 (br s, 1H), 6.17-6.08 |
| | | | | (m, 1H), 4.65-4.59 (m, 2H), 4.56-4.13 (m, 4H), 3.78 (s, 3H), |
| | | | | 3.71 (d, J = 3.2 Hz, 3H), 3.67-3.58 (m, 2H), 3.20 (br t, J = |
| | | | | 12.0 Hz, 2H), 3.09 (q, J = 6.8 Hz, 3H), 2.37-2.24 (m, 2H), |
| | | | | 1.77 (br d, J = 9.6 Hz, 2H), 1.64-1.52 (m, 2H) |
| I-423 | EK | D | 668.2 | 12.13 (d, J = 7.2 Hz, 1H), 8.14-8.07 (m, 1H), 7.93-7.48 (m, |
| | | | | 1H), 7.43 (dd, J = 1.2, 8.0 Hz, 1H), 7.38 (d, J = 8.0 Hz, 1H), |
| | | | | 7.35 (dd, J = 1.6, 7.6 Hz, 1H), 7.32-7.28 (m, 1H), 7.19-7.14 |
| | | | | (m, 2H), 7.09-7.06 (m, 1H), 7.06-7.03 (m, 1H), 6.97-6.89 |
| | | | | (m, 1H), 6.45 (s, 1H), 6.16-6.10 (m, 1H), 4.65-4.59 (m, 2H), |
| | | | | 4.39-4.30 (m, 2H), 3.84 (s, 4H), 3.74-3.72 (m, 3H), 3.65 (t, J = |
| | | | | 5.6 Hz, 1H), 3.60 (t, J = 5.6 Hz, 1H), 3.09 (q, J = 6.8 Hz, |
| | | | | 2H), 3.01 (s, 4H), 2.36-2.26 (m, 2H) |
| I-424 | EX | D | 708.3 | 12.37-11.98 (m, 1H), 8.31-8.07 (m, 1H), 7.71 (t, J = 11.6 |
| | | | | Hz, 1H), 7.45-7.29 (m, 2H), 7.21-7.02 (m, 4H), 7.00-6.87 |
| | | | | (m, 1H), 6.87-6.69 (m, 1H), 6.51-6.35 (m, 1H), 6.22-6.09 |
| | | | | (m, 1H), 5.48 (d, J = 2.8 Hz, 4H), 4.73-4.57 (m, 2H), 4.47- |
| | | | | 4.27 (m, 2H), 3.89 (s, 2H), 3.72 (s, 3H), 3.69-3.50 (m, 2H), |
| | | | | 3.09 (d, J = 7.2 Hz, 3H), 3.04 (s, 2H), 2.47-2.21 (m, 2H), 0.92- |
| | | | | 0.61 (m, 4H) |
| I-425 | FJ | D | 662.4 | 12.17-12.11 (m, 1H), 8.14-8.10 (m, 2H), 7.71-7.67 (m, 1H), |
| | | | | 7.46 (br d, J = 8.0 Hz, 1H), 7.43-7.39 (m, 1H), 7.38-7.36 (m, |
| | | | | 1H), 7.28-7.25 (m, 1H), 7.17 (br d, J = 8.0 Hz, 1H), 7.08- |
| | | | | 7.04 (m, 1H), 6.98-6.91 (m, 1H), 6.63-6.53 (m, 1H), 6.49 (br |
| | | | | s, 1H), 6.16-6.12 (m, 1H), 4.65-4.61 (m, 2H), 4.35-4.35 (m, |
| | | | | 1H), 4.39 (br s, 3H), 3.86 (br s, 2H), 3.82 (s, 3H), 3.75-3.74 |
| | | | | (m, 3H), 3.66 (br t, J = 5.2 Hz, 1H), 3.61 (s, 1H), 3.10 (br d, J = |
| | | | | 6.4 Hz, 2H), 2.67 (br s, 2H), 2.36 (br d, J = 3.2 Hz, 1H), 2.29 |
| | | | | (br s, 1H) |
| I-426 | AU | FN | 701.2 | 12.21-12.08 (m, 1H), 8.23-8.17 (m, 1H), 8.14-8.04 (m, 2H), |
| | | | | 7.81-7.47 (m, 2H), 7.12-7.03 (m, 1H), 7.00-6.93 (m, 2H), |
| | | | | 6.92-6.86 (m, 2H), 6.60 (s, 1H), 6.46-6.40 (m, 1H), 6.18- |
| | | | | 6.09 (m, 1H), 4.63 (q, J = 7.2 Hz, 2H), 4.39-4.30 (m, 2H), |
| | | | | 3.83 (s, 4H), 3.80 (s, 3H), 3.68-3.59 (m, 2H), 3.13-3.06 (m, |
| | | | | 2H), 3.00 (s, 4H), 2.38-2.27 (m, 2H) |
| I-427 | FU | D | 700.3 | 12.18-12.10 (m, 1H), 8.15-8.08 (m, 1H), 7.74-7.65 (m, 1H), |
| | | | | 7.42-7.38 (m, 1H), 7.35 (dd, J = 1.6, 7.6 Hz, 1H), 7.32-7.08 |
| | | | | (m, 3H), 7.07-7.01 (m, 1H), 6.99-6.90 (m, 1H), 6.82 (dd, J = |
| | | | | 2.0, 8.4 Hz, 1H), 6.71 (s, 1H), 6.62-6.56 (m, 1H), 6.47-6.43 |
| | | | | (m, 1H), 6.18-6.11 (m, 1H), 4.66-4.60 (m, 2H), 4.40-4.31 |
| | | | | (m, 2H), 3.81 (br s, 4H), 3.75 (d, J = 3.2 Hz, 3H), 3.68-3.59 |
| | | | | (m, 2H), 3.26 (br d, J = 4.4 Hz, 4H), 3.10 (q, J = 6.8 Hz, 2H), |
| | | | | 2.37-2.27 (m, 2H) |
| I-428 | BO | BM | 684.2 | 12.19 (br d, J = 8.0 Hz, 1H), 8.50 (br d, J = 5.6 Hz, 1H), 8.41 |
| | | | | (s, 1H), 8.13-8.07 (m, 1H), 7.78 (d, J = 2.4 Hz, 1H), 7.72- |
| | | | | 7.63 (m, 1H), 7.36 (dd, J = 2.4, 10.4 Hz, 1H), 7.21 (br d, J = |
| | | | | 5.2 Hz, 1H), 7.04-6.93 (m, 1H), 6.51-6.43 (m, 1H), 6.18- |
| | | | | 6.09 (m, 1H), 4.65-4.59 (m, 2H), 4.39-4.30 (m, 2H), 3.84 (s, |
| | | | | 3H), 3.82 (br d, J = 2.8 Hz, 4H), 3.80 (br s, 3H), 3.66-3.58 |
| | | | | (m, 2H), 3.26 (br s, 4H), 3.09 (q, J = 6.4 Hz, 2H), 2.36-2.26 |
| | | | | (m, 2H) |
| I-429 | FX | BM | 667.3 | 12.24-12.16 (m, 1H), 8.50 (d, J = 5.6 Hz, 1H), 8.42 (s, 1H), |
| | | | | 8.27-8.21 (m, 1H), 8.13-8.05 (m, 2H), 7.68 (d, J = 12.0 Hz, |
| | | | | 1H), 7.21 (d, J = 5.6 Hz, 1H), 7.05-6.92 (m, 1H), 6.50 (br s, |
| | | | | 1H), 6.18-6.11 (m, 1H), 4.62 (q, J = 7.2 Hz, 2H), 4.39-4.31 |
| | | | | (m, 2H), 3.84 (s, 3H), 3.83 (d, J = 2.8 Hz, 3H), 3.76 (br d, J = |
| | | | | 10.0 Hz, 8H), 3.65 (br t, J = 5.6 Hz, 1H), 3.60 (br t, J = 5.6 Hz, |
| | | | | 1H), 3.10 (q, J = 6.4 Hz, 2H), 2.36-2.25 (m, 2H) |
| I-430 | GH | D | 719.1 | 12.14 (br d, J = 7.2 Hz, 1H), 8.13-8.09 (m, 1H), 7.74-7.67 |
| | | | | (m, 2H), 7.41-7.37 (m, 1H), 7.35 (dd, J = 1.2, 7.6 Hz, 1H), |
| | | | | 7.15 (br d, J = 8.4 Hz, 1H), 7.05 (t, J = 7.6 Hz, 1H), 6.97-6.90 |
| | | | | (m, 1H), 6.79 (d, J = 8.4 Hz, 1H), 6.47 (br s, 1H), 6.43 (d, J = |
| | | | | 7.6 Hz, 1H), 6.16-6.11 (m, 1H), 4.64-4.59 (m, 2H), 4.38- |
| | | | | 4.31 (m, 2H), 3.78 (br s, 4H), 3.74 (d, J = 3.2 Hz, 3H), 3.60 (br |
| | | | | s, 4H), 3.09 (br d, J = 6.8 Hz, 2H), 2.54 (s, 2H), 2.35 (br s, 1H), |
| | | | | 2.27 (br dd, J = 1.2, 2.4 Hz, 1H) |
| I-431 | GI | D | 682.4 | 12.13 (d, J = 1.6 Hz, 1H), 8.14-8.09 (m, 1H), 7.75-7.62 (m, |
| | | | | 1H), 7.42-7.37 (m, 1H), 7.34 (d, J = 7.6 Hz, 1H), 7.15 (d, J = |
| | | | | 8.4 Hz, 1H), 7.05 (t, J = 7.2 Hz, 1H), 6.97-6.90 (m, 1H), 6.89 |
| | | | | (s, 1H), 6.87 (dd, J = 2.0, 8.8 Hz, 1H), 6.68 (dt, J = 2.4, 8.6 Hz, |
| | | | | 1H), 6.42 (s, 1H), 6.15-6.10 (m, 1H), 4.65-4.59 (m, 2H), |
| | | | | 4.38-4.31 (m, 2H), 3.79 (s, 6H), 3.72 (d, J = 3.2 Hz, 3H), 3.67- |
| | | | | 3.63 (m, 1H), 3.60 (t, J = 5.6 Hz, 1H), 3.12-3.07 (m, 2H), |
| | | | | 2.93 (s, 4H), 2.34 (d, J = 3.2 Hz, 2H), 2.27 (d, J = 2.0 Hz, 1H) |
| I-432 | GJ | D | 700.3 | 12.14 (d, J = 7.2 Hz, 1H), 8.13-8.08 (m, 1H), 7.74-7.63 (m, |
| | | | | 1H), 7.41-7.36 (m, 1H), 7.34 (d, J = 7.6 Hz, 1H), 7.14 (d, J = |
| | | | | 8.0 Hz, 1H), 7.04 (t, J = 7.2 Hz, 1H), 6.97-6.89 (m, 1H), 6.81- |
| | | | | 6.74 (m, 2H), 6.40 (s, 1H), 6.18-6.07 (m, 1H), 4.65-4.59 |
| | | | | (m, 2H), 4.38-4.30 (m, 2H), 3.80 (s, 3H), 3.74 (s, 3H), 3.71 |
| | | | | (d, J = 3.2 Hz, 3H), 3.68-3.56 (m, 3H), 3.09 (d, J = 6.8 Hz, |
| | | | | 2H), 3.02 (s, 4H), 2.34 (s, 1H), 2.27 (s, 1H) |
| I-433 | AW | D | 680.4 | 12.26-12.15 (m, 1H), 8.55-8.38 (m, 2H), 8.18-8.05 (m, 1H), |
| | | | | 7.75-7.62 (m, 1H), 7.27-7.12 (m, 2H), 7.06-6.91 (m, 1H), |
| | | | | 6.74 (d, J = 8.0 Hz, 1H), 6.48 (d, J = 2.4 Hz, 1H), 6.23-6.11 |
| | | | | (m, 1H), 4.66-4.56 (m, 2H), 4.40-4.30 (m, 2H), 3.82 (d, J = |
| | | | | 3.2 Hz, 3H), 3.78 (s, 4H), 3.76 (s, 3H), 3.65 (t, J = 5.6 Hz, 1H), |
| | | | | 3.60 (t, J = 5.6 Hz, 1H), 3.32 (s, 5H), 3.09 (q, J = 6.8 Hz, 2H), |
| | | | | 2.35 (s, 1H), 2.28 (s, 3H) |
| I-434 | GM | BM | 681.2 | 12.25 (d, J = 8.0 Hz, 1H), 8.61-8.45 (m, 2H), 8.15-8.09 (m, |
| | | | | 1H), 7.74-7.57 (m, 2H), 7.30 (s, 1H), 7.11 (dd, J = 2.4, 7.6 |
| | | | | Hz, 1H), 6.99 (d, J = 6.0 Hz, 1H), 6.82-6.63 (m, 1H), 6.61- |
| | | | | 6.52 (m, 1H), 6.16 (s, 1H), 4.68-4.60 (m, 2H), 4.47-4.41 (m, |
| | | | | 1H), 4.39-4.32 (m, 2H), 3.96 (s, 3H), 3.89 (d, J = 4.4 Hz, 4H), |
| | | | | 3.68-3.60 (m, 2H), 3.14-3.08 (m, 2H), 2.64 (s, 2H), 2.55 (s, |
| | | | | 2H), 2.37-2.29 (m, 2H) |
| I-435 | GN | BJ | 734.3 | 12.24 (s, 1H), 8.50 (s, 1H), 8.30 (d, J = 4.8 Hz, 1H), 8.17-8.05 |
| | | | | (m, 2H), 7.74-7.57 (m, 1H), 7.47-7.43 (m, 1H), 7.43-7.38 |
| | | | | (m, 1H), 7.10-6.97 (m, 1H), 6.54 (s, 1H), 6.19-6.11 (m, 1H), |
| | | | | 4.62 (q, J = 7.2 Hz, 2H), 4.41-4.30 (m, 2H), 3.88 (s, 3H), 3.86 |
| | | | | (d, J = 3.2 Hz, 3H), 3.80 (s, 4H), 3.68-3.59 (m, 2H), 3.56 (s, |
| | | | | 4H), 3.14-3.06 (m, 2H), 2.37-2.26 (m, 2H) |
| I-436 | AX | BJ | 695.3 | 12.26-12.17 (m, 1H), 8.55-8.47 (m, 1H), 8.32-8.27 (m, 1H), |
| | | | | 8.13-8.07 (m, 1H), 7.71-7.63 (m, 1H), 7.54 (d, J = 2.4 Hz, |
| | | | | 1H), 7.41 (dd, J = 2.0, 4.4 Hz, 1H), 7.09-6.99 (m, 1H), 6.97 |
| | | | | (d, J = 2.4 Hz, 1H), 6.53-6.50 (m, 1H), 6.18-6.11 (m, 1H), |
| | | | | 4.62 (q, J = 7.2 Hz, 2H), 4.38-4.30 (m, 2H), 3.86 (d, J = 3.2 |
| | | | | Hz, 3H), 3.79 (d, J = 12.8 Hz, 9H), 3.67-3.59 (m, 2H), 3.23 |
| | | | | (d, J = 9.2 Hz, 1H), 3.18 (s, 4H), 3.09 (d, J = 8.4 Hz, 2H), 2.36- |
| | | | | 2.26 (m, 2H) |
| I-437 | AX | BM | 696.2 | 12.47-11.97 (m, 1H), 8.50 (d, J = 5.8 Hz, 1H), 8.41 (s, 1H), |
| | | | | 8.13-8.08 (m, 1H), 7.71-7.64 (m, 1H), 7.53 (d, J = 2.4 Hz, |
| | | | | 1H), 7.21 (d, J = 5.6 Hz, 1H), 7.03-6.93 (m, 2H), 6.47 (d, J = |
| | | | | 3.2 Hz, 1H), 6.20-6.12 (m, 1H), 4.62 (q, J = 7.2 Hz, 2H), 4.39- |
| | | | | 4.31 (m, 2H), 3.84-3.79 (m, 8H), 3.77 (s, 6H), 3.67-3.58 |
| | | | | (m, 2H), 3.17 (br s, 3H), 3.09 (br d, J = 6.4 Hz, 2H), 2.36- |
| | | | | 2.26 (m, 2H) |
| I-438 | AZ | BM | 706.3 | 12.29-12.04 (m, 1H), 8.50 (d, J = 5.6 Hz, 1H), 8.41 (s, 1H), |
| | | | | 8.13-8.07 (m, 1H), 7.74-7.65 (m, 1H), 7.62 (d, J = 2.0 Hz, |
| | | | | 1H), 7.17 (s, 1H), 7.04-6.92 (m, 1H), 6.91-6.87 (m, 1H), |
| | | | | 6.46 (br s, 1H), 6.20-6.10 (m, 1H), 4.65-4.59 (m, 2H), 4.39- |
| | | | | 4.30 (m, 2H), 3.82 (d, J = 3.2 Hz, 3H), 3.79 (s, 7H), 3.66-3.58 |
| | | | | (m, 2H), 3.25 (br s, 4H), 3.09 (q, J = 6.4 Hz, 2H), 2.36-2.25 |
| | | | | (m, 2H), 1.91-1.81 (m, 1H), 0.94-0.87 (m, 2H), 0.73-0.65 |
| | | | | (m, 2H) |
| I-439 | GP | D | 664.4 | 12.12 (d, J = 1.6 Hz, 1H), 8.13-8.08 (m, 1H), 7.72-7.63 (m, |
| | | | | 1H), 7.55 (dd, J = 1.6, 4.8 Hz, 1H), 7.41-7.34 (m, 2H), 7.15 |
| | | | | (d, J = 8.0 Hz, 1H), 7.05 (t, J = 7.2 Hz, 1H), 6.97-6.90 (m, |
| | | | | 2H), 6.81 (dd, J = 1.2, 8.0 Hz, 1H), 6.41 (s, 1H), 6.16-6.10 |
| | | | | (m, 1H), 5.14-5.09 (m, 1H), 4.65-4.59 (m, 2H), 4.38-4.31 |
| | | | | (m, 2H), 3.86 (s, 4H), 3.72 (d, J = 3.6 Hz, 3H), 3.65 (t, J = 5.2 |
| | | | | Hz, 1H), 3.60 (t, J = 5.6 Hz, 1H), 3.09 (q, J = 6.8 Hz, 2H), 2.98 |
| | | | | (s, 4H), 2.73 (d, J = 5.2 Hz, 3H), 2.36-2.26 (m, 2H) |
| I-440 | GU | BM | 666.3 | 12.23 (d, J = 6.8 Hz, 1H), 8.50 (d, J = 6.0 Hz, 1H), 8.42 (s, |
| | | | | 1H), 8.11 (d, J = 11.2 Hz, 1H), 7.72-7.64 (m, 1H), 7.47 (t, J = |
| | | | | 8.0 Hz, 1H), 7.21 (d, J = 6.0 Hz, 1H), 7.04-6.95 (m, 1H), 6.50 |
| | | | | (s, 1H), 6.33 (d, J = 8.0 Hz, 1H), 6.18-6.13 (m, 1H), 6.07 (d, J = |
| | | | | 8.0 Hz, 1H), 4.65-4.60 (m, 2H), 4.39-4.31 (m, 2H), 3.84 |
| | | | | (s, 2H), 3.83 (s, 2H), 3.77 (s, 4H), 3.68-3.61 (m, 2H), 3.60- |
| | | | | 3.54 (m, 6H), 3.12-3.08 (m, 2H), 2.35 (s, 1H) |
| I-441 | FX | BM | 667.3 | 12.26-12.15 (m, 1H), 8.51 (d, J = 5.6 Hz, 1H), 8.42 (s, 1H), |
| | | | | 8.15-8.07 (m, 1H), 7.78 (d, J = 2.8 Hz, 1H), 7.73-7.66 (m, |
| | | | | 1H), 7.65 (d, J = 3.2 Hz, 1H), 7.22 (d, J = 5.6 Hz, 1H), 7.05- |
| | | | | 6.94 (m, 1H), 6.50 (s, 1H), 6.15 (s, 1H), 4.63 (q, J = 7.6 Hz, |
| | | | | 2H), 4.40-4.31 (m, 2H), 3.92 (s, 3H), 3.83 (d, J = 3.2 Hz, 4H), |
| | | | | 3.81 (d, J = 2.0 Hz, 2H), 3.68-3.60 (m, 2H), 3.51 (s, 3H), 3.14- |
| | | | | 3.08 (m, 2H), 2.42 (s, 1H), 2.39-2.32 (m, 2H), 2.31-2.25 |
| | | | | (m, 1H) |
| I-442 | GV | BM | 681.2 | 12.30-12.15 (m, 1H), 8.51 (br d, J = 4.8 Hz, 1H), 8.43 (s, 1H), |
| | | | | 8.15-8.08 (m, 2H), 7.72-7.64 (m, 1H), 7.55-7.46 (m, 1H), |
| | | | | 7.21 (br d, J = 5.2 Hz, 1H), 7.04-6.94 (m, 1H), 6.62-6.44 (m, |
| | | | | 2H), 6.19-6.11 (m, 1H), 4.66-4.59 (m, 2H), 4.43-4.28 (m, |
| | | | | 4H), 3.84 (br s, 9H), 3.70-3.63 (m, 1H), 3.60 (br s, 1H), 3.10 |
| | | | | (br d, J = 5.2 Hz, 1H), 2.64 (br s, 2H), 2.36-2.27 (m, 2H) |
| I-443 | GW | BM | 663.1 | 12.26-12.18 (m, 1H), 8.51 (br d, J = 5.0 Hz, 1H), 8.44-8.41 |
| | | | | (m, 1H), 8.14-8.06 (m, 1H), 7.71-7.63 (m, 2H), 7.24-7.18 |
| | | | | (m, 1H), 7.09 (br d, J = 7.4 Hz, 1H), 7.04-6.95 (m, 1H), 6.84- |
| | | | | 6.72 (m, 1H), 6.69 (br d, J = 7.8 Hz, 1H), 6.53 (br s, 1H), 6.20- |
| | | | | 6.12 (m, 1H), 4.62 (br d, J = 7.0 Hz, 2H), 4.46-4.31 (m, 4H), |
| | | | | 3.85 (br s, 9H), 3.66-3.58 (m, 2H), 3.09 (br s, 1H), 2.65 (br s, |
| | | | | 2H), 2.33-2.27 (m, 2H) |
| I-444 | GX | BM | 684.3 | 12.28 (d, J = 8.8 Hz, 1H), 8.63 (s, 1H), 8.59-8.53 (m, 1H), |
| | | | | 8.14-8.09 (m, 1H), 7.78-7.60 (m, 1H), 7.49 (dd, J = 8.8, 10.4 |
| | | | | Hz, 1H), 7.43-7.35 (m, 1H), 7.09-7.01 (m, 1H), 6.56 (s, 1H), |
| | | | | 6.29 (dd, J = 1.6, 8.8 Hz, 1H), 6.20-6.15 (m, 1H), 4.63 (q, J = |
| | | | | 6.8 Hz, 2H), 4.39-4.32 (m, 2H), 3.91 (d, J = 5.6 Hz, 2H), 3.87 |
| | | | | (s, 3H), 3.79 (s, 4H), 3.67-3.60 (m, 2H), 3.52 (s, 5H), 3.13- |
| | | | | 3.07 (m, 2H), 2.37-2.28 (m, 2H) |
| I-445 | GN | BM | 734.2 | 12.28-12.12 (m, 1H), 8.51 (d, J = 5.6 Hz, 1H), 8.42 (s, 1H), |
| | | | | 8.18-8.06 (m, 2H), 7.74-7.62 (m, 1H), 7.46 (d, J = 1.6 Hz, |
| | | | | 1H), 7.22 (d, J = 5.6 Hz, 1H), 7.07-6.91 (m, 1H), 6.50 (br s, |
| | | | | 1H), 6.23-6.10 (m, 1H), 4.63 (q, J = 7.2 Hz, 2H), 4.40-4.31 |
| | | | | (m, 2H), 3.89 (s, 3H), 3.83 (br d, J = 3.2 Hz, 3H), 3.81 (br s, |
| | | | | 4H), 3.68-3.55 (m, 6H), 3.14-3.08 (m, 2H), 2.38-2.27 (m, 2H) |
| I-446 | GN | GY | 784.3 | 12.46-12.09 (m, 1H), 8.89-8.83 (m, 1H), 8.27 (d, J = 8.0 Hz, |
| | | | | 1H), 8.13-8.05 (m, 3H), 7.81 (t, J = 7.2 Hz, 1H), 7.70 (s, 1H), |
| | | | | 7.68-7.65 (m, 1H), 7.43 (s, 1H), 7.25-7.12 (m, 1H), 6.61 (d, J = |
| | | | | 3.2 Hz, 1H), 6.21 (s, 1H), 4.66-4.60 (m, 2H), 4.43-4.35 |
| | | | | (m, 2H), 3.86 (s, 3H), 3.79 (s, 4H), 3.67-3.60 (m, 2H), 3.53 (s, |
| | | | | 7H), 3.13-3.08 (m, 2H), 2.38-2.28 (m, 2H) |
| I-447 | G | GY | 742.3 | 12.45-12.28 (m, 1H), 8.89-8.82 (m, 1H), 8.31-8.23 (m, 1H), |
| | | | | 8.15-8.03 (m, 2H), 7.85-7.76 (m, 2H), 7.71-7.63 (m, 2H), |
| | | | | 7.51 (dd, J = 1.2, 8.0 Hz, 1H), 7.24-7.13 (m, 1H), 6.91 (dd, J = |
| | | | | 4.8, 8.0 Hz, 1H), 6.62-6.56 (m, 1H), 6.25-6.19 (m, 1H), |
| | | | | 4.62 (q, J = 6.8 Hz, 2H), 4.44-4.36 (m, 2H), 3.86 (tt, J = 2.8, |
| | | | | 5.6 Hz, 1H), 3.76 (s, 4H), 3.68-3.60 (m, 2H), 3.53 (s, 3H), |
| | | | | 3.28-3.25 (m, 4H), 3.14-3.08 (m, 2H), 2.37-2.28 (m, 2H), |
| | | | | 0.81-0.74 (m, 2H), 0.72-0.65 (m, 2H) |
| I-448 | AU | GY | 715.3 | 12.50-12.19 (m, 1H), 8.85 (s, 1H), 8.27 (d, J = 8.4 Hz, 1H), |
| | | | | 8.15-8.01 (m, 2H), 7.87-7.77 (m, 1H), 7.74-7.60 (m, 2H), |
| | | | | 7.25-7.14 (m, 1H), 6.97-6.91 (m, 2H), 6.86 (s, 2H), 6.60 (d, J = |
| | | | | 2.8 Hz, 1H), 6.26-6.18 (m, 1H), 4.62 (q, J = 6.8 Hz, 2H), |
| | | | | 4.43-4.35 (m, 2H), 3.80 (s, 4H), 3.76 (s, 3H), 3.67-3.60 (m, |
| | | | | 2H), 3.53 (s, 3H), 3.13-3.08 (m, 2H), 2.96 (s, 4H), 2.38-2.28 |
| | | | | (m, 2H) |
| I-449 | P | GY | 649.3 | 12.44-12.27 (m, 1H), 8.85 (s, 1H), 8.27 (d, J = 8.0 Hz, 1H), |
| | | | | 8.13-8.04 (m, 2H), 7.82 (t, J = 7.2 Hz, 1H), 7.72-7.63 (m, |
| | | | | 2H), 7.24-7.14 (m, 1H), 6.52 (s, 1H), 6.25-6.18 (m, 1H), |
| | | | | 4.65-4.58 (m, 2H), 4.44-4.35 (m, 2H), 3.70-3.61 (m, 2H), |
| | | | | 3.60 (d, J = 2.4 Hz, 4H), 3.52 (s, 3H), 3.36-3.33 (m, 4H), 3.13- |
| | | | | 3.07 (m, 2H), 2.37-2.27 (m, 2H), 1.66-1.58 (m, 1H), 0.44- |
| | | | | 0.37 (m, 2H), 0.31 (d, J = 2.8 Hz, 2H) |
| I-450 | HD | BM | 699.3 | 12.25-12.18 (m, 1H), 8.52-8.49 (m, 1H), 8.41 (s, 1H), 8.13- |
| | | | | 8.08 (m, 1H), 7.70-7.66 (m, 1H), 7.21 (br d, J = 5.2 Hz, 1H), |
| | | | | 7.00 (br s, 1H), 6.97-6.88 (m, 3H), 6.47 (br s, 1H), 6.15 (br |
| | | | | dd, J = 3.2, 13.2 Hz, 1H), 4.65-4.60 (m, 2H), 4.39-4.31 (m, |
| | | | | 2H), 3.81 (br s, 11H), 3.65 (br s, 1H), 3.60 (br s, 1H), 3.09 (br |
| | | | | d, J = 5.6 Hz, 2H), 2.97 (br s, 3H), 2.34 (br d, J = 4.4 Hz, 1H), |
| | | | | 2.29-2.24 (m, 1H) |
| I-451 ^{f} | FJ | GY | 713.4 | 9.60-9.40 (m, 1H), 8.90 (d, J = 4.8 Hz, 1H), 8.32 (d, J = 8.4 |
| | | | | Hz, 1H), 8.22-8.06 (m, 2H), 7.82-7.75 (m, 2H), 7.69-7.59 |
| | | | | (m, 2H), 7.20-7.14 (m, 3H), 6.79-6.72 (m, 1H), 6.70-6.32 |
| | | | | (m, 1H), 6.27-6.16 (m, 1H), 4.83-4.78 (m, 2H), 4.53 (s, 2H), |
| | | | | 4.39-4.33 (m, 1H), 4.09-4.00 (m, 2H), 3.83 (d, J = 0.8 Hz, |
| | | | | 3H), 3.82-3.78 (m, 1H), 3.63-3.60 (m, 1H), 3.57 (d, J = 7.2 |
| | | | | Hz, 3H), 3.09 (t, J = 6.4 Hz, 2H), 2.80 (s, 2H), 2.40 (s, 2H), |
| | | | | 1.26 (s, 1H) |
| I-452 | FJ | BM | 663.3 | 12.24 (s, 1H), 8.52 (d, J = 5.6 Hz, 1H), 8.44 (s, 1H), 8.15-8.09 |
| | | | | (m, 2H), 7.72-7.65 (m, 1H), 7.46 (d, J = 8.4 Hz, 1H), 7.27 |
| | | | | (dd, J = 4.4, 8.4 Hz, 1H), 7.22 (d, J = 5.6 Hz, 1H), 6.97 (s, 1H), |
| | | | | 6.53 (s, 2H), 6.15 (s, 1H), 4.66-4.60 (m, 2H), 4.41-4.32 (m, |
| | | | | 3H), 3.88-3.80 (m, 9H), 3.66-3.59 (m, 2H), 3.14-3.08 (m, |
| | | | | 2H), 2.67 (d, J = 2.0 Hz, 2H), 2.38-2.28 (m, 2H) |
| I-453 | HF | BM | 694.2 | 12.25-12.15 (m, 1H), 8.51 (d, J = 5.6 Hz, 1H), 8.42 (s, 1H), |
| | | | | 8.18-8.08 (m, 1H), 7.73-7.62 (m, 2H), 7.22 (d, J = 5.6 Hz, |
| | | | | 1H), 7.16 (d, J = 1.6 Hz, 1H), 7.08-6.94 (m, 1H), 6.48 (s, 1H), |
| | | | | 6.20-6.11 (m, 1H), 4.68-4.60 (m, 2H), 4.41-4.32 (m, 2H), |
| | | | | 3.83 (d, J = 3.2 Hz, 3H), 3.81 (s, 3H), 3.78 (s, 4H), 3.67-3.59 |
| | | | | (m, 2H), 3.27 (s, 4H), 3.13-3.09 (m, 2H), 2.55-2.53 (m, 2H), |
| | | | | 2.36-2.26 (m, 2H), 1.19-1.15 (m, 3H) |
| I-454 | HG | BM | 708.2 | 12.21 (d, J = 1.2 Hz, 1H), 8.51 (d, J = 5.6 Hz, 1H), 8.42 (s, |
| | | | | 1H), 8.14-8.05 (m, 1H), 7.72-7.63 (m, 2H), 7.21 (d, J = 5.6 |
| | | | | Hz, 1H), 7.16 (d, J = 1.6 Hz, 1H), 6.96 (d, J = 6.0 Hz, 1H), |
| | | | | 6.48 (s, 1H), 6.14 (s, 1H), 4.66-4.59 (m, 2H), 4.41-4.29 (m, |
| | | | | 2H), 3.83 (s, 3H), 3.82 (s, 3H), 3.79 (s, 3H), 3.68-3.59 (m, |
| | | | | 2H), 3.27-3.26 (m, 4H), 3.13-3.09 (m, 2H), 2.91-2.62 (m, |
| | | | | 2H), 2.36-2.27 (m, 2H), 1.21 (d, J = 6.8 Hz, 6H) |
| I-455 | HH | BM | 710.3 | 12.20 (d, J = 2.4 Hz, 1H), 8.51 (d, J = 4.8 Hz, 1H), 8.42 (s, |
| | | | | 1H), 8.15-8.09 (m, 1H), 7.74-7.64 (m, 1H), 7.25-7.19 (m, |
| | | | | 1H), 7.08-7.04 (m, 1H), 7.02-6.94 (m, 1H), 6.47 (s, 1H), |
| | | | | 6.19-6.12 (m, 1H), 4.67-4.60 (m, 2H), 4.42-4.32 (m, 2H), |
| | | | | 3.83 (s, 6H), 3.80-3.77 (m, 7H), 3.66 (t, J = 5.6 Hz, 1H), 3.63- |
| | | | | 3.60 (m, 1H), 3.16 (s, 4H), 3.13-3.09 (m, 2H), 2.34 (d, J = |
| | | | | 6.4 Hz, 2H), 2.20 (s, 3H), 1.24 (s, 2H) |
| I-456 | G | BM | 692.2 | 12.42-11.86 (m, 1H), 8.50 (d, J = 5.6 Hz, 1H), 8.41 (s, 1H), |
| | | | | 8.14-8.08 (m, 1H), 7.81 (d, J = 4.4 Hz, 1H), 7.71-7.64 (m, |
| | | | | 1H), 7.53 (d, J = 8.0 Hz, 1H), 7.21 (br d, J = 5.6 Hz, 1H), 7.03- |
| | | | | 6.91 (m, 2H), 6.47 (br s, 1H), 6.18-6.12 (m, 1H), 4.65-4.59 |
| | | | | (m, 2H), 4.37 (br s, 1H), 4.32 (br s, 1H), 3.88 (td, J = 3.2 5.6 |
| | | | | Hz, 1H), 3.82 (d, J = 2.8 Hz, 3H), 3.77 (br s, 4H), 3.66-3.58 |
| | | | | (m, 2H), 3.29-3.26 (m, 4H), 3.09 (q, J = 6.4 Hz, 2H), 2.36- |
| | | | | 2.27 (m, 2H), 0.80 (br d, J = 5.6 Hz, 2H), 0.70 (br s, 2H) |
| I-457 | BN | BJ | 700.4 | 12.26 (br d, J = 8.4 Hz, 1H), 8.51 (s, 1H), 8.31 (d, J = 4.8 Hz, |
| | | | | 1H), 8.13-8.09 (m, 1H), 7.82 (d, J = 1.6 Hz, 1H), 7.70-7.66 |
| | | | | (m, 1H), 7.43-7.39 (m, 2H), 7.09-7.00 (m, 1H), 6.53 (br d, J = |
| | | | | 2.0 Hz, 1H), 6.17-6.12 (m, 1H), 4.64-4.60 (m, 2H), 4.37- |
| | | | | 4.30 (m, 2H), 3.86 (d, J = 3.2 Hz, 3H), 3.84 (s, 3H), 3.79 (br d, |
| | | | | J = 2.8 Hz, 4H), 3.65 (br t, J = 5.2 Hz, 1H), 3.61-3.58 (m, |
| | | | | 1H), 3.12-3.08 (m, 2H), 2.43 (br s, 4H), 2.35 (br d, J = 3.6 Hz, |
| | | | | 1H), 2.30-2.25 (m, 1H) |
| I-458 | BN | BM | 700.3 | 12.21 (br d, J = 6.0 Hz, 1H), 8.51 (d, J = 5.6 Hz, 1H), 8.42 (s, |
| | | | | 1H), 8.13-8.09 (m, 1H), 7.82 (d, J = 2.0 Hz, 1H), 7.71-7.67 |
| | | | | (m, 1H), 7.40 (d, J = 2.0 Hz, 1H), 7.21 (d, J = 5.6 Hz, 1H), 7.04- |
| | | | | 6.94 (m, 1H), 6.48 (br s, 1H), 6.19-6.13 (m, 1H), 4.65-4.60 |
| | | | | (m, 2H), 4.39-4.31 (m, 2H), 3.84 (s, 3H), 3.82 (d, J = 3.1 Hz, |
| | | | | 3H), 3.79 (br s, 4H), 3.67-3.63 (m, 1H), 3.60 (br t, J = 5.6 Hz, |
| | | | | 1H), 3.37-3.36 (m, 2H), 3.10 (q, J = 6.8 Hz, 2H), 2.54 (s, 2H), |
| | | | | 2.36-2.33 (m, 1H), 2.31-2.25 (m, 1H) |
| I-459 | BN | HJ | 717.8 | 12.14 (br d, J = 8.0 Hz, 1H), 8.15-8.07 (m, 1H), 7.82 (d, J = |
| | | | | 2.0 Hz, 1H), 7.74-7.63 (m, 1H), 7.40-7.33 (m, 2H), 7.05 (br |
| | | | | d, J = 11.2 Hz, 1H), 6.95-6.84 (m, 2H), 6.44 (br s, 1H), 6.18- |
| | | | | 6.07 (m, 1H), 4.62 (q, J = 7.2 Hz, 2H), 4.37-4.28 (m, 2H), |
| | | | | 3.84 (s, 3H), 3.82-3.71 (m, 8H), 3.66-3.58 (m, 2H), 3.31- |
| | | | | 3.27 (m, 3H), 3.09 (q, J = 7.2 Hz, 2H), 2.36-2.25 (m, 2H), |
| | | | | 1.75 (s, 1H) |
| I-460 | BN | HM | 700.6 | 12.23-12.17 (m, 1H), 8.24-8.22 (m, 1H), 8.13-8.09 (m, 1H), |
| | | | | 7.83-7.81 (m, 1H), 7.81-7.78 (m, 1H), 7.70-7.66 (m, 1H), |
| | | | | 7.39 (d, J = 2.4 Hz, 1H), 7.11 (dd, J = 5.2, 7.2 Hz, 1H), 7.05- |
| | | | | 6.96 (m, 1H), 6.50 (d, J = 2.4 Hz, 1H), 6.18-6.10 (m, 1H), |
| | | | | 4.59 (s, 3H), 4.38-4.30 (m, 2H), 3.84 (s, 6H), 3.79 (br s, 4H), |
| | | | | 3.70-3.56 (m, 3H), 3.09 (q, J = 7.2 Hz, 3H), 2.40-2.31 (m, |
| | | | | 2H), 2.30-2.24 (m, 1H) |
| I-461 | BN | HK | 735.4 | 12.25-12.09 (m, 1H), 8.19-8.05 (m, 1H), 7.82 (d, J = 2.0 Hz, |
| | | | | 1H), 7.76-7.62 (m, 1H), 7.49-7.42 (m, 1H), 7.40 (d, J = 2.0 |
| | | | | Hz, 1H), 7.29 (br dd, J = 7.2, 12.8 Hz, 1H), 7.01-6.89 (m, |
| | | | | 1H), 6.48 (br s, 1H), 6.20-6.09 (m, 1H), 4.62 (q, J = 6.8 Hz, |
| | | | | 2H), 4.38-4.29 (m, 2H), 3.92-3.68 (m, 13H), 3.67-3.57 (m, |
| | | | | 3H), 3.12-3.06 (m, 2H), 2.36-2.25 (m, 2H) |
| I-462 | BN | HL | 735.3 | 12.35-12.25 (m, 1H), 8.16-8.07 (m, 1H), 7.87-7.66 (m, 2H), |
| | | | | 7.44-7.37 (m, 2H), 7.17-7.00 (m, 2H), 6.58 (s, 1H), 6.22- |
| | | | | 6.13 (m, 1H), 4.63 (q, J = 6.8 Hz, 2H), 4.40-4.32 (m, 2H), |
| | | | | 3.85 (s, 3H), 3.79 (s, 4H), 3.68-3.59 (m, 3H), 3.54 (d, J = 3.2 |
| | | | | Hz, 3H), 3.31-3.26 (m, 2H), 3.10 (q, J = 7.2 Hz, 2H), 2.42- |
| | | | | 2.32 (m, 2H), 2.29 (d, J = 2.4 Hz, 1H) |
| I-463 | HN | BM | 733.2 | 12.36-12.31 (m, 1H), 8.75 (d, J = 5.2 Hz, 1H), 8.71-8.66 (m, |
| | | | | 1H), 8.13-8.08 (m, 1H), 7.74-7.65 (m, 1H), 7.59-7.53 (m, |
| | | | | 1H), 7.24 (d, J = 8.0 Hz, 1H), 7.19 (d, J = 1.6 Hz, 1H), 7.13- |
| | | | | 7.03 (m, 2H), 6.61 (s, 1H), 6.20-6.14 (m, 1H), 4.65-4.60 (m, |
| | | | | 2H), 4.39-4.31 (m, 2H), 4.00-3.97 (m, 3H), 3.87 (s, 3H), |
| | | | | 3.83 (s, 4H), 3.66 (t, J = 5.2 Hz, 1H), 3.60 (t, J = 5.6 Hz, 1H), |
| | | | | 3.13-3.07 (m, 6H), 2.37-2.27 (m, 2H) |
| I-464 | LZ | D | 595.2 | 12.12 (d, J = 1.6 Hz, 1H), 8.17-8.07 (m, 1H), 7.76-7.63 (m, |
| | | | | 1H), 7.46-7.37 (m, 2H), 7.30-7.22 (m, 1H), 7.18 (d, J = 8.4 |
| | | | | Hz, 1H), 7.07 (t, J = 7.6 Hz, 1H), 7.01-6.91 (m, 1H), 6.76 (d, |
| | | | | J = 8.8 Hz, 1H), 6.14 (d, J = 1.6 Hz, 1H), 5.02 (s, 1H), 4.81 (d, |
| | | | | J = 18.8 Hz, 2H), 4.67-4.60 (m, 3H), 4.41-4.32 (m, 2H), 3.81 |
| | | | | (s, 3H), 3.77-3.74 (m, 3H), 3.68-3.59 (m, 2H), 3.14-3.08 |
| | | | | (m, 2H), 2.36-2.28 (m, 2H) |
| I-465 | HQ | D | 665.3 | 12.38-11.96 (m, 1H), 8.22-8.09 (m, 2H), 8.04 (s, 1H), 7.74- |
| | | | | 7.63 (m, 1H), 7.47-7.32 (m, 2H), 7.16 (d, J = 8.4 Hz, 1H), |
| | | | | 7.10-7.02 (m, 1H), 7.00 (d, J = 5.6 Hz, 1H), 6.97-6.89 (m, |
| | | | | 1H), 6.50-6.39 (m, 1H), 6.21-6.05 (m, 1H), 4.69-4.54 (m, |
| | | | | 2H), 4.42-4.29 (m, 2H), 3.88 (s, 3H), 3.84 (s, 3H), 3.76 (d, J = |
| | | | | 3.6 Hz, 4H), 3.68 (t, J = 5.8 Hz, 1H), 3.60 (s, 1H), 3.14-3.09 |
| | | | | (m, 2H), 3.08 (s, 4H), 2.37-2.26 (m, 2H) |
| I-466 | HS | BM | 694.3 | 12.20 (d, J = 7.6 Hz, 1H), 8.50 (d, J = 5.6 Hz, 1H), 8.42 (s, |
| | | | | 1H), 8.14-8.08 (m, 1H), 7.72-7.65 (m, 1H), 7.21 (d, J = 6.0 |
| | | | | Hz, 1H), 7.17 (d, J = 8.0 Hz, 1H), 7.03-6.94 (m, 1H), 6.74 (d, |
| | | | | J = 8.0 Hz, 1H), 6.48 (s, 1H), 6.18-6.13 (m, 1H), 4.65-4.59 |
| | | | | (m, 2H), 4.39-4.31 (m, 2H), 3.82 (d, J = 3.2 Hz, 3H), 3.78 (s, |
| | | | | 4H), 3.76 (s, 3H), 3.65 (t, J = 5.6 Hz, 1H), 3.60 (t, J = 5.6 Hz, |
| | | | | 1H), 3.35-3.34 (m, 2H), 3.10 (q, J = 6.4 Hz, 2H), 2.60-2.51 |
| | | | | (m, 4H), 2.36-2.26 (m, 2H), 1.16 (t, J = 7.6 Hz, 3H) |
| I-467 | HT | BM | 667.2 | 12.27-12.14 (m, 1H), 8.64 (d, J = 5.6 Hz, 1H), 8.50 (d, J = 5.6 |
| | | | | Hz, 1H), 8.42 (s, 1H), 8.13-8.09 (m, 1H), 7.71-7.66 (m, 1H), |
| | | | | 7.21 (d, J = 5.6 Hz, 1H), 7.09 (d, J = 5.6 Hz, 1H), 7.04-6.95 |
| | | | | (m, 1H), 6.49 (br s, 1H), 6.16 (br d, J = 12.0 Hz, 1H), 4.62 (br |
| | | | | d, J = 7.6 Hz, 2H), 4.39-4.31 (m, 2H), 3.88 (s, 3H), 3.83 (br d, |
| | | | | J = 2.8 Hz, 8H), 3.66-3.59 (m, 2H), 3.44 (br s, 3H), 3.09 (br d, |
| | | | | J = 6.4 Hz, 2H), 2.35 (br d, J = 2.4 Hz, 1H), 2.28 (br s, 1H) |
| I-468 ^{f} | BO | GY | 734.3 | 9.55-9.42 (m, 1H), 8.88 (d, J = 6.0 Hz, 1H), 8.39-8.14 (m, |
| | | | | 2H), 7.82 (d, J = 6.4 Hz, 1H), 7.76 (d, J = 2.4 Hz, 1H), 7.72 (d, |
| | | | | J = 2.0 Hz, 1H), 7.67 (d, J = 4.4 Hz, 2H), 7.17-7.11 (m, 1H), |
| | | | | 6.92-6.83 (m, 1H), 6.64 (s, 1H), 6.21 (d, J = 18.0 Hz, 1H), |
| | | | | 4.84-4.77 (m, 2H), 4.53-4.32 (m, 2H), 4.00 (s, 4H), 3.87 (s, |
| | | | | 3H), 3.80 (t, J = 6.0 Hz, 1H), 3.60 (d, J = 6.4 Hz, 4H), 3.37 (s, |
| | | | | 4H), 3.09 (t, J = 6.0 Hz, 2H), 2.40 (s, 2H) |
| I-469 ^{f} | GX | GY | 734.3 | 9.57-9.44 (m, 1H), 8.89 (d, J = 6.0 Hz, 1H), 8.33 (d, J = 8.4 |
| | | | | Hz, 1H), 8.28-8.16 (m, 1H), 7.83 (t, J = 7.2 Hz, 1H), 7.78- |
| | | | | 7.72 (m, 1H), 7.70-7.62 (m, 2H), 7.22 (s, 1H), 7.16 (dd, J = |
| | | | | 2.8, 6.0 Hz, 1H), 6.67 (d, J = 7.6 Hz, 1H), 6.21 (d, J = 18.4 Hz, |
| | | | | 1H), 6.05 (d, J = 8.4 Hz, 1H), 4.83-4.78 (m, 2H), 4.52 (s, 1H), |
| | | | | 4.34 (s, 1H), 3.99 (s, 4H), 3.94 (s, 3H), 3.80 (t, J = 5.6 Hz, 1H), |
| | | | | 3.61 (d, J = 7.2 Hz, 4H), 3.55 (s, 4H), 3.09 (t, J = 6.0 Hz, 2H), |
| | | | | 2.40 (s, 2H) |
| I-470 | HU | HV | 681.3 | 12.23 (d, J = 9.6 Hz, 1H), 8.47 (d, J = 5.6 Hz, 1H), 8.43 (s, |
| | | | | 2H), 8.13-8.08 (m, 2H), 7.74-7.63 (m, 1H), 7.19 (d, J = 5.6 |
| | | | | Hz, 1H), 7.07-6.99 (m, 1H), 6.54 (s, 1H), 6.16 (d, J = 11.2 |
| | | | | Hz, 1H), 4.62 (q, J = 7.2 Hz, 2H), 4.39-4.32 (m, 2H), 4.19- |
| | | | | 4.13 (m, 2H), 3.96 (s, 3H), 3.82 (s, 4H), 3.68-3.57 (m, 4H), |
| | | | | 3.10 (s, 2H), 3.09 (s, 2H), 2.36-2.26 (m, 2H), 1.23-1.17 (m, 3H) |
| I-471 | AU | HZ | 663.3 | 12.35 (s, 1H), 8.54 (d, J = 5.2 Hz, 1H), 8.42 (s, 1H), 8.14-8.08 |
| | | | | (m, 1H), 7.74-7.66 (m, 1H), 7.43 (d, J = 5.2 Hz, 1H), 6.99- |
| | | | | 6.90 (m, 3H), 6.89-6.86 (m, 2H), 6.36 (s, 1H), 6.16 (s, 1H), |
| | | | | 4.67-4.60 (m, 2H), 4.41-4.33 (m, 2H), 3.79 (s, 7H), 3.67- |
| | | | | 3.59 (m, 2H), 3.27 (s, 2H), 3.13-3.08 (m, 2H), 2.97 (s, 4H), |
| | | | | 2.37-2.27 (m, 2H), 1.02 (t, J = 7.6 Hz, 3H) |
| I-472 | BN | HZ | 698.3 | 12.33 (s, 1H), 8.54 (d, J = 5.2 Hz, 1H), 8.42 (s, 1H), 8.14-8.08 |
| | | | | (m, 1H), 7.82 (d, J = 2.0 Hz, 1H), 7.71-7.65 (m, 1H), 7.43 (d, |
| | | | | J = 5.2 Hz, 1H), 7.40 (d, J = 2.0 Hz, 1H), 6.99-6.86 (m, 1H), |
| | | | | 6.36 (s, 1H), 6.16 (s, 1H), 4.62 (q, J = 6.8 Hz, 2H), 4.40-4.32 |
| | | | | (m, 2H), 3.84 (s, 3H), 3.76 (s, 4H), 3.68-3.59 (m, 2H), 3.31- |
| | | | | 3.28 (m, 6H), 3.13-3.08 (m, 2H), 2.36-2.28 (m, 2H), 1.02 (t, |
| | | | | J = 7.6 Hz, 3H) |
| I-473 | P | HZ | 597.3 | 12.33 (s, 1H), 8.53 (d, J = 4.0 Hz, 1H), 8.44-8.36 (m, 1H), |
| | | | | 8.17-8.05 (m, 1H), 7.75-7.64 (m, 1H), 7.43 (d, J = 4.4 Hz, |
| | | | | 1H), 7.02-6.83 (m, 1H), 6.28 (s, 1H), 6.17 (d, J = 1.6 Hz, 1H), |
| | | | | 4.67-4.56 (m, 2H), 4.41-4.31 (m, 2H), 3.69-3.55 (m, 6H), |
| | | | | 3.30 (s, 4H), 3.13-3.05 (m, 2H), 2.68 (dd, J = 1.6, 3.6 Hz, |
| | | | | 1H), 2.41-2.25 (m, 3H), 1.64 (dd, J = 2.0, 6.0 Hz, 1H), 1.06- |
| | | | | 0.97 (m, 3H), 0.47-0.30 (m, 4H) |
| I-474 ^{f} | IA | BM | 690.3 | 9.45 (s, 1H), 8.61-8.50 (m, 2H), 7.76 (d, J = 3.6 Hz, 1H), |
| | | | | 7.67 (d, J = 3.2 Hz, 1H), 7.25 (d, J = 1.6 Hz, 1H), 7.09 (d, J = |
| | | | | 1.6 Hz, 1H), 7.05-7.00 (m, 2H), 6.89 (d, J = 8.4 Hz, 1H), 6.60- |
| | | | | 6.55 (m, 1H), 6.16 (d, J = 16.8 Hz, 1H), 4.83-4.78 (m, 2H), |
| | | | | 4.48 (s, 1H), 4.30 (d, J = 1.6 Hz, 1H), 4.04 (s, 4H), 3.91 (d, J = |
| | | | | 10.4 Hz, 6H), 3.79 (t, J = 6.0 Hz, 1H), 3.60 (t, J = 6.0 Hz, 1H), |
| | | | | 3.21 (s, 4H), 3.11-3.05 (m, 2H), 2.38 (s, 2H) |
| I-475 ^{f} | IB | BM | 683.2 | 9.43 (s, 1H), 8.62-8.47 (m, 2H), 7.79-7.63 (m, 2H), 7.03- |
| | | | | 6.96 (m, 3H), 6.69 (t, J = 8.0 Hz, 1H), 6.59-6.54 (m, 2H), |
| | | | | 6.16 (d, J = 17.2 Hz, 1H), 4.80 (q, J = 6.0 Hz, 2H), 4.49 (s, |
| | | | | 1H), 4.30 (d, J = 1.2 Hz, 1H), 4.04 (s, 4H), 3.89 (d, J = 2.0 Hz, |
| | | | | 6H), 3.79 (t, J = 6.0 Hz, 1H), 3.59 (t, J = 6.0 Hz, 1H), 3.15 (s, |
| | | | | 4H), 3.07 (q, J = 6.4 Hz, 2H), 2.38 (s, 2H) |
| I-476 | BO | HZ | 682.3 | 12.40-12.25 (m, 1H), 8.54 (d, J = 4.8 Hz, 1H), 8.42 (s, 1H), |
| | | | | 8.15-8.08 (m, 1H), 7.79 (d, J = 2.4 Hz, 1H), 7.72-7.65 (m, |
| | | | | 1H), 7.43 (d, J = 5.2 Hz, 1H), 7.36 (dd, J = 2.4, 10.4 Hz, 1H), |
| | | | | 6.89 (dd, J = 2.0, 4.0 Hz, 1H), 6.35 (s, 1H), 6.17 (d, J = 5.2 Hz, |
| | | | | 1H), 4.62 (q, J = 6.8 Hz, 2H), 4.41-4.33 (m, 2H), 3.84 (s, 3H), |
| | | | | 3.77 (s, 4H), 3.67-3.58 (m, 2H), 3.25 (d, J = 1.2 Hz, 4H), 3.10 |
| | | | | (d, J = 5.2 Hz, 2H), 2.39-2.26 (m, 4H), 1.02 (t, J = 7.6 Hz, 3H) |
| I-477 | G | ID | 717.5 | 12.17-11.97 (m, 1H), 8.11 (d, J = 10.8 Hz, 1H), 7.82 (dd, J = |
| | | | | 1.2, 4.8 Hz, 1H), 7.72-7.65 (m, 1H), 7.55-7.51 (m, 1H), 7.47- |
| | | | | 7.38 (m, 2H), 7.37-7.33 (m, 1H), 7.07 (t, J = 6.8 Hz, 1H), |
| | | | | 6.95-6.86 (m, 2H), 6.41 (t, J = 3.2 Hz, 1H), 6.16-6.09 (m, |
| | | | | 1H), 4.65-4.59 (m, 2H), 4.38-4.30 (m, 2H), 3.88 (td, J = 2.8, |
| | | | | 5.6 Hz, 1H), 3.79 (br s, 5H), 3.65 (br t, J = 5.6 Hz, 1H), 3.59 |
| | | | | (br t, J = 5.6 Hz, 1H), 3.30-3.27 (m, 4H), 3.09 (q, J = 7.2 Hz, |
| | | | | 2H), 2.34 (br s, 1H), 2.30-2.24 (m, 1H), 0.85-0.78 (m, 2H), |
| | | | | 0.76-0.69 (m, 4H), 0.58-0.53 (m, 2H) |
| I-478 | MA | HV | 681.3 | 12.20 (d, J = 9.2 Hz, 1H), 8.50-8.38 (m, 2H), 8.15-8.07 (m, |
| | | | | 1H), 7.77 (d, J = 2.8 Hz, 1H), 7.73-7.61 (m, 2H), 7.19 (d, J = |
| | | | | 6.0 Hz, 1H), 7.09-6.97 (m, 1H), 6.54 (d, J = 2.0 Hz, 1H), 6.21- |
| | | | | 6.11 (m, 1H), 4.66-4.58 (m, 2H), 4.40-4.30 (m, 2H), 4.20- |
| | | | | 4.12 (m, 2H), 3.91 (s, 3H), 3.80 (s, 4H), 3.67-3.58 (m, 2H), |
| | | | | 3.49 (s, 4H), 3.13-3.06 (m, 2H), 2.37-2.26 (m, 2H), 1.24- |
| | | | | 1.16 (m, 3H) |
| I-479 | G | HZ | 690.2 | 12.33 (s, 1H), 8.54 (d, J = 4.8 Hz, 1H), 8.42 (s, 1H), 8.15-8.07 |
| | | | | (m, 1H), 7.81 (d, J = 4.8 Hz, 1H), 7.73-7.66 (m, 1H), 7.56- |
| | | | | 7.51 (m, 1H), 7.43 (d, J = 5.2 Hz, 1H), 6.97-6.88 (m, 2H), |
| | | | | 6.35 (s, 1H), 6.17 (s, 1H), 4.67-4.60 (m, 2H), 4.41-4.33 (m, |
| | | | | 2H), 3.88 (d, J = 2.8 Hz, 1H), 3.75 (s, 4H), 3.67-3.59 (m, 2H), |
| | | | | 3.29-3.26 (m, 4H), 3.11-3.07 (m, 2H), 2.39-2.29 (m, 4H), |
| | | | | 1.02 (t, J = 7.6 Hz, 3H), 0.80 (d, J = 6.4 Hz, 2H), 0.70 (s, 2H) |
| I-480 | GU | HZ | 664.3 | 12.40-12.29 (m, 1H), 8.54 (d, J = 4.8 Hz, 1H), 8.43 (s, 1H), |
| | | | | 8.16-8.05 (m, 1H), 7.75-7.62 (m, 1H), 7.50-7.41 (m, 2H), |
| | | | | 6.90 (d, J = 6.4 Hz, 1H), 6.40-6.29 (m, 2H), 6.17 (s, 1H), 6.07 |
| | | | | (d, J = 8.0 Hz, 1H), 4.63 (q, J = 6.8 Hz, 2H), 4.41-4.32 (m, |
| | | | | 2H), 3.80-3.72 (m, 7H), 3.67-3.59 (m, 2H), 3.56 (s, 4H), |
| | | | | 3.12-3.08 (m, 2H), 2.38-2.31 (m, 4H), 1.03 (t, J = 7.6 Hz, 3H) |
| I-481 | II | BM | 705.2 | 12.21 (br d, J = 6.4 Hz, 1H), 8.51 (d, J = 5.6 Hz, 1H), 8.42 (d, J = |
| | | | | 1.6 Hz, 1H), 8.13-8.09 (m, 1H), 7.71-7.67 (m, 1H), 7.21 |
| | | | | (d, J = 6.0 Hz, 1H), 7.03-6.95 (m, 1H), 6.77 (d, J = 8.0 Hz, |
| | | | | 1H), 6.66 (d, J = 1.6 Hz, 1H), 6.58 (dd, J = 1.6, 8.4 Hz, 1H), |
| | | | | 6.47 (br s, 1H), 6.18-6.13 (m, 1H), 4.63 (br d, J = 8.0 Hz, |
| | | | | 2H), 4.39-4.32 (m, 2H), 3.83 (d, J = 3.2 Hz, 3H), 3.80 (br s, |
| | | | | 4H), 3.78 (s, 3H), 3.66 (br t, J = 5.6 Hz, 1H), 3.60 (s, 1H), 3.10 |
| | | | | (br d, J = 6.4 Hz, 2H), 2.93 (br s, 4H), 2.35 (br d, J = 5.6 Hz, |
| | | | | 1H), 2.28 (br d, J = 1.6 Hz, 1H), 1.88-1.83 (m, 1H), 0.90- |
| | | | | 0.86 (m, 2H), 0.62 (dd, J = 2.0, 4.8 Hz, 2H) |
| I-482 | GI | BM | 683.1 | 12.21 (d, J = 7.6 Hz, 1H), 8.50 (d, J = 5.6 Hz, 1H), 8.41 (s, |
| | | | | 1H), 8.14-8.07 (m, 1H), 7.71-7.65 (m, 1H), 7.21 (d, J = 6.0 |
| | | | | Hz, 1H), 7.05-6.93 (m, 1H), 6.92-6.83 (m, 2H), 6.68 (dt, J = |
| | | | | 2.8, 8.4 Hz, 1H), 6.47 (s, 1H), 6.19-6.10 (m, 1H), 4.66-4.58 |
| | | | | (m, 2H), 4.40-4.29 (m, 2H), 3.82 (d, J = 3.2 Hz, 4H), 3.80 (s, |
| | | | | 6H), 3.65 (t, J = 5.6 Hz, 1H), 3.60 (t, J = 5.6 Hz, 1H), 3.09 (q, |
| | | | | J = 6.4 Hz, 2H), 2.93 (s, 4H), 2.38-2.33 (m, 1H), 2.27 (s, 1H) |
| I-483 | KM | HV | 681.3 | 12.22 (br d, J = 9.2 Hz, 1H), 8.56 (d, J = 5.2 Hz, 1H), 8.47 (d, J = |
| | | | | 5.6 Hz, 2H), 8.13-8.08 (m, 1H), 7.68 (d, J = 11.6 Hz, 1H), |
| | | | | 7.19 (d, J = 5.6 Hz, 1H), 7.07-6.99 (m, 1H), 6.90 (d, J = 5.6 |
| | | | | Hz, 1H), 6.55 (br s, 1H), 6.18-6.13 (m, 1H), 4.65-4.60 (m, |
| | | | | 2H), 4.39-4.32 (m, 2H), 4.18-4.14 (m, 2H), 4.02 (s, 3H), |
| | | | | 3.81 (br s, 5H), 3.65-3.60 (m, 2H), 3.32 (br s, 3H), 3.09 (br d, |
| | | | | J = 7.2 Hz, 2H), 2.35 (br d, J = 0.8 Hz, 1H), 2.28 (br s, 1H), |
| | | | | 1.23-1.17 (m, 3H) |
| I-484 | AX | LA | 694.6 | 12.40-12.28 (m, 1H), 8.60 (s, 1H), 8.47 (d, J = 4.8 Hz, 1H), |
| | | | | 8.14-8.02 (m, 1H), 7.73-7.63 (m, 1H), 7.53 (d, J = 2.4 Hz, |
| | | | | 1H), 7.28 (br d, J = 4.8 Hz, 1H), 6.98-6.87 (m, 2H), 6.37 (br |
| | | | | d, J = 2.4 Hz, 1H), 6.20-6.12 (m, 1H), 4.62 (q, J = 7.2 Hz, |
| | | | | 2H), 4.39-4.30 (m, 2H), 3.80 (s, 3H), 3.77 (s, 7H), 3.15 (br s, |
| | | | | 4H), 3.12-3.08 (m, 2H), 2.56 (br s, 3H), 2.37-2.31 (m, 2H), |
| | | | | 2.27 (br s, 1H), 0.97 (br t, J = 7.4 Hz, 3H) |
| I-485 | LE | BM | 700.2 | 12.32-12.15 (m, 1 H), 12.06 (s, 1 H), 8.53 (d, J = 5.6 Hz, 1 |
| | | | | H), 8.45 (s, 1 H), 8.11 (d, J = 11.2 Hz, 1 H), 7.94 (s, 1 H), 7.79 |
| | | | | (t, J = 2.8 Hz, 1 H), 7.68 (d, J = 12.0 Hz, 1 H), 7.24 (d, J = 5.6 |
| | | | | Hz, 1 H), 7.08-6.91 (m, 1 H), 6.52 (br dd, J = 1.6, 3.2 Hz, 2 |
| | | | | H), 6.21-6.09 (m, 1 H), 4.62 (q, J = 7.2 Hz, 2 H), 4.42-4.29 |
| | | | | (m, 2 H), 3.89 (s, 4 H), 3.84 (d, J = 3.6 Hz, 3 H), 3.67-3.57 |
| | | | | (m, 2 H), 3.15-3.03 (m, 6 H), 2.36-2.26 (m, 2 H) |
| I-486 | HU | LH | 680.3 | 12.22 (d, J = 8.0 Hz, 1H), 8.47 (d, J = 5.6 Hz, 1H), 8.42 (s, |
| | | | | 2H), 8.10 (s, 1H), 7.77-7.67 (m, 1H), 7.48-7.31 (m, 1H), |
| | | | | 7.19 (d, J = 5.6 Hz, 1H), 7.06-6.98 (m, 1H), 6.54 (d, J = 3.2 |
| | | | | Hz, 1H), 6.22-6.13 (m, 2H), 4.39-4.35 (m, 3H), 4.34-4.29 |
| | | | | (m, 1H), 4.20-4.14 (m, 2H), 3.96 (s, 3H), 3.82 (s, 4H), 3.65 (t, |
| | | | | J = 5.6 Hz, 1H), 3.58 (t, J = 5.6 Hz, 1H), 3.11 (s, 4H), 3.00- |
| | | | | 2.95 (m, 2H), 2.34-2.25 (m, 2H), 1.23-1.17 (m, 3H) |
| I-487 | Q | LI | 667.3 | 12.10 (br s, 1H), 8.50 (d, J = 5.6 Hz, 1H), 8.40 (s, 1H), 7.79 |
| | | | | (dd, J = 1.2, 4.8 Hz, 1H), 7.71 (dd, J = 2.0, 10.0 Hz, 1H), 7.41 |
| | | | | (dd, J = 1.2, 15.6 Hz, 1H), 7.26 (dd, J = 1.2, 8.0 Hz, 1H), 7.20 |
| | | | | (d, J = 6.0 Hz, 1H), 7.00 (d, J = 5.6 Hz, 1H), 6.91 (dd, J = 4.8, |
| | | | | 8.0 Hz, 1H), 6.45 (br s, 1H), 6.20 (td, J = 2.0, 10.0 Hz, 1H), |
| | | | | 4.53-4.45 (m, 1H), 4.38-4.32 (m, 2H), 3.88 (br d, J = 14.0 |
| | | | | Hz, 1H), 3.85-3.70 (m, 11H), 3.30-3.24 (m, 2H), 3.24-3.00 |
| | | | | (m, 2H), 2.99-2.93 (m, 1H), 2.93-2.90 (m, 1H), 2.89-2.72 |
| | | | | (m, 1H), 2.65-2.53 (m, 1H), 1.95-1.83 (m, 2H), 1.79-1.71 |
| | | | | (m, 1H), 1.56-1.39 (m, 1H) |
| I-488 ^{g} | BO | LJ | 343.8 | 12.23-11.99 (m, 1H), 8.51 (d, J = 5.6 Hz, 1H), 8.40 (s, 1H), |
| | | | | 8.11 (d, J = 11.2 Hz, 1H), 7.79 (d, J = 2.4 Hz, 1H), 7.70 (d, J = |
| | | | | 10.2 Hz, 1H), 7.37 (dd, J = 2.4, 10.2 Hz, 1H), 7.21 (d, J = 6.0 |
| | | | | Hz, 1H), 7.02 (t, J = 4.8 Hz, 1H), 6.46 (br s, 1H), 4.62 (q, J = |
| | | | | 6.8 Hz, 2H), 4.53-4.46 (m, 1H), 3.91 (br d, J = 13.6 Hz, 1H), |
| | | | | 3.85-3.79 (m, 10H), 3.26 (br s, 3H), 3.12-2.98 (m, 4H), 2.90- |
| | | | | 2.72 (m, 1H), 2.65-2.53 (m, 1H), 1.97-1.87 (m, 2H), 1.78 |
| | | | | (br d, J = 12.8 Hz, 1H), 1.59-1.42 (m, 1H) |
| I-489 | Q | LJ | 668.2 | 12.18-12.01 (m, 1H), 8.51 (d, J = 5.6 Hz, 1H), 8.41 (s, 1H), |
| | | | | 8.11 (d, J = 11.2 Hz, 1H), 7.80 (d, J = 4.8 Hz, 1H), 7.70 (d, J = |
| | | | | 10.4 Hz, 1H), 7.27 (d, J = 8.0 Hz, 1H), 7.21 (d, J = 5.6 Hz, |
| | | | | 1H), 7.02 (t, J = 4.8 Hz, 1H), 6.92 (dd, J = 4.8, 8.0 Hz, 1H), |
| | | | | 6.46 (br s, 1H), 4.62 (q, J = 6.4 Hz, 2H), 4.54-4.45 (m, 1H), |
| | | | | 3.94-3.88 (m, 1H), 3.84-3.77 (m, 10H), 3.36-3.33 (m, 3H), |
| | | | | 3.30-3.20 (m, 1H), 3.14-2.94 (m, 4H), 2.82-2.60 (m, 1H), |
| | | | | 1.98-1.85 (m, 2H), 1.78 (br d, J = 13.6 Hz, 1H), 1.61-1.36 |
| | | | | (m, 1H) |
| I-490 | LM | D | 731.1 | 12.13 (br d, J = 8.0 Hz, 1H), 8.13-8.09 (m, 1H), 7.70 (s, 1H), |
| | | | | 7.55 (s, 1H), 7.40 (br d, J = 4.0 Hz, 1H), 7.39-7.37 (m, 1H), |
| | | | | 7.37-7.34 (m, 1H), 7.15 (br d, J = 8.4 Hz, 1H), 7.05 (br t, J = |
| | | | | 7.2 Hz, 1H), 6.98-6.90 (m, 1H), 6.49 (d, J = 8.4 Hz, 1H), 6.44 |
| | | | | (br s, 1H), 6.13 (br d, J = 11.2 Hz, 1H), 4.62 (br d, J = 7.2 Hz, |
| | | | | 2H), 4.38 (br s, 2H), 3.78 (s, 4H), 3.74-3.72 (m, 3H), 3.65 (br |
| | | | | d, J = 4.0 Hz, 1H), 3.60 (br s, 1H), 3.44 (br s, 4H), 3.12-3.07 |
| | | | | (m, 2H), 2.54 (s, 3H), 2.35 (br s, 1H), 2.27 (br s, 1H) |
| I-491 | LP | BM | 700.2 | 12.20 (br d, J = 7.6 Hz, 1H), 8.50 (d, J = 5.6 Hz, 1H), 8.42 (s, |
| | | | | 1H), 8.14-8.08 (m, 1H), 7.74-7.65 (m, 1H), 7.32 (d, J = 8.4 |
| | | | | Hz, 1H), 7.21 (br d, J = 5.6 Hz, 1H), 7.03-6.92 (m, 2H), 6.49 |
| | | | | (br s, 1H), 6.18-6.12 (m, 1H), 4.62 (q, J = 7.6 Hz, 2H), 4.39- |
| | | | | 4.31 (m, 2H), 3.84-3.78 (m, 10H), 3.67-3.58 (m, 2H), 3.39 |
| | | | | (br s, 4H), 3.10 (q, J = 6.4 Hz, 2H), 2.36-2.27 (m, 2H) |
| I-492 | LU | BM | 701.6 | 12.38-12.18 (m, 1 H), 8.71-8.48 (m, 2 H), 8.13-8.07 (m, 1 |
| | | | | H), 7.71-7.65 (m, 1 H), 7.44-7.36 (m, 1 H), 7.10-6.96 (m, 2 |
| | | | | H), 6.78 (dt, J = 5.6, 9.2 Hz, 1 H), 6.55 (br s, 1 H), 6.20-6.12 |
| | | | | (m, 1 H), 4.66-4.58 (m, 2 H), 4.40-4.28 (m, 2 H), 3.95-3.88 |
| | | | | (m, 6 H), 3.82 (s, 4 H), 3.70-3.53 (m, 2 H), 3.13-3.06 (m, 2 |
| | | | | H), 3.04-2.95 (m, 4 H), 2.35-2.34 (m, 1 H), 2.30-2.23 (m, 1H) |
| I-493 | LV | BM | 661.2 | 12.24 (br d, J = 6.8 Hz, 1H), 8.52 (br d, J = 5.6 Hz, 1H), 8.43 |
| | | | | (s, 1H), 8.31 (dd, J = 1.2, 4.4 Hz, 1H), 8.17-8.01 (m, 1H), |
| | | | | 7.77-7.54 (m, 3H), 7.30-7.15 (m, 1H), 7.08-6.88 (m, 1H), |
| | | | | 6.62-6.41 (m, 1H), 6.26-6.01 (m, 1H), 4.62 (q, J = 7.2 Hz, |
| | | | | 2H), 4.41-4.28 (m, 2H), 3.88 (br s, 4H), 3.84 (d, J = 3.6 Hz, |
| | | | | 3H), 3.67-3.56 (m, 2H), 3.29-3.26 (m, 4H), 3.12-3.07 (m, |
| | | | | 2H), 2.36-2.26 (m, 2H) |
| I-494 | LE | AG | 698.2 | 12.17 (d, J = 9.2 Hz, 1 H) 12.08 (s, 1 H) 7.94 (s, 1 H) 7.80 (s, 1 |
| | | | | H) 7.68-7.75 (m, 1 H) 7.29-7.46 (m, 3 H) 7.15 (br d, J = 8.4 |
| | | | | Hz, 1 H) 7.05 (t, J = 7.2 Hz, 1 H) 6.86-7.00 (m, 1 H) 6.50 (d, |
| | | | | J = 16.8 Hz, 2 H) 6.15-6.23 (m, 1 H) 6.11 (s, 1 H) 4.32-4.40 |
| | | | | (m, 3 H) 4.29 (s, 1 H) 3.89 (s, 4 H) 3.73 (s, 3 H) 3.61-3.67 (m, |
| | | | | 1 H) 3.58-3.57 (m, 1 H) 3.09 (s, 4 H) 2.97 (m, 2 H) 2.19-2.39 |
| | | | | (m, 2 H) |
| I-495 | LW | D | 675.2 | 12.13 (d, J = 5.6 Hz, 1H), 8.12-8.07 (m, 1H), 8.05 (dd, J = |
| | | | | 1.6, 4.8 Hz, 1H), 7.72-7.63 (m, 1H), 7.42-7.36 (m, 1H), 7.34 |
| | | | | (d, J = 7.2 Hz, 1H), 7.21 (dd, J = 1.6, 7.6 Hz, 1H), 7.15 (d, J = |
| | | | | 8.0 Hz, 1H), 7.05 (t, J = 7.2 Hz, 1H), 6.98-6.92 (m, 1H), 6.91 |
| | | | | (s, 1H), 6.43 (s, 1H), 6.16-6.09 (m, 1H), 4.68-4.57 (m, 2H), |
| | | | | 4.40-4.28 (m, 2H), 3.84 (s, 4H), 3.72 (d, J = 3.2 Hz, 3H), 3.63 |
| | | | | (d, J = 16.8 Hz, 2H), 3.22 (s, 4H), 3.09 (q, J = 6.8 Hz, 2H), |
| | | | | 2.37-2.24 (m, 2H), 2.07-1.98 (m, 1H), 1.05-0.98 (m, 2H), |
| | | | | 0.71 (d, J = 4.0 Hz, 2H) |
| I-496 | LZ | BM | 596.2 | 12.23 (d, J = 2.8 Hz, 1H), 8.60-8.36 (m, 2H), 8.11 (d, J = 11.2 |
| | | | | Hz, 1H), 7.76-7.59 (m, 1H), 7.37-7.15 (m, 2H), 7.12-6.88 |
| | | | | (m, 1H), 6.86-6.71 (m, 1H), 6.17 (d, J = 7.2 Hz, 1H), 5.03 (s, |
| | | | | 1H), 4.88-4.75 (m, 2H), 4.62 (s, 3H), 4.44-4.27 (m, 2H), |
| | | | | 3.87 (d, J = 17.6 Hz, 3H), 3.82-3.72 (m, 3H), 3.69-3.56 (m, |
| | | | | 2H), 3.15-3.06 (m, 2H), 2.40-2.23 (m, 2H) |
| I-497 | Q | MF | 682.3 | 1.03 (m, 3 H) 2.04-2.11 (m, 3 H) 2.24-2.41 (m, 2 H) 2.53 (br |
| | | | | d, J = 7.6 Hz, 2 H) 3.07 (br s, 4 H) 3.58-3.64 (m, 2 H) 3.79 (br |
| | | | | s, 4 H) 3.85-3.88 (m, 3 H) 4.31-4.38 (m, 2 H) 6.14-6.23 (m, |
| | | | | 1 H) 6.39 (d, J = 2.4 Hz, 1 H) 6.89-7.01 (m, 1 H) 7.18 (m, 1 H) |
| | | | | 7.53 (br d, J = 4.8 Hz, 1 H) 7.72 (d, J = 2.4 Hz, 1 H) 8.09-8.17 |
| | | | | (m, 1 H) 8.48 (s, 1 H) 8.56-8.61 (m, 1 H) 12.37 (s, 1 H) |
| I-498 | MS | MX | 596.3 | 12.33-12.14 (m, 1H), 8.54 (d, J = 5.2 Hz, 1H), 8.46-8.40 (m, |
| | | | | 1H), 8.10 (d, J = 8.0 Hz, 1H), 7.69 (d, J = 9.6 Hz, 1H), 7.47- |
| | | | | 7.42 (m, 1H), 7.28-7.19 (m, 1H), 6.99-6.92 (m, 1H), 6.65 (s, |
| | | | | 1H), 4.96-4.76 (m, 3H), 4.64-4.58 (m, 3H), 4.51-4.45 (m, |
| | | | | 1H), 3.94-3.86 (m, 1H), 3.80 (s, 2H), 3.72 (s, 1H), 3.29-2.94 |
| | | | | (m, 6H), 2.80-2.60 (m, 1H), 1.94-1.84 (m, 2H), 1.76 (d, J = |
| | | | | 13.2 Hz, 1H), 1.58-1.42 (m, 1H), 1.06-0.99 (m, 3H) |
| I-499 | BO | BQ | 611.3 | 12.52 (br d, J = 3.6 Hz, 1 H) 8.15 (s, 1 H) 8.11 (d, J = 8.0 Hz, 1 |
| | | | | H) 7.80 (d, J = 2.4 Hz, 1 H) 7.69 (d, J = 8.4 Hz, 1 H) 7.38 (m, 1 |
| | | | | H) 7.04-7.18 (m, 1 H) 6.80 (br s, 1 H) 6.07-6.19 (m, 1 H) |
| | | | | 4.62 (m, 2 H) 4.30 (br d, J = 18.8 Hz, 2 H) 3.85 (s, 3 H) 3.82 (br |
| | | | | s, 4 H) 3.55-3.67 (m, 2 H) 3.29 (br s, 4 H) 3.05-3.14 (m, 2 |
| | | | | H) 2.20-2.39 (m, 2 H) |
| I-500 | MZ | BM | 698.2 | 12.24-12.16 (m, 1H), 8.50 (d, J = 5.6 Hz, 1H), 8.41 (s, 1H), |
| | | | | 8.15-8.06 (m, 1H), 7.71-7.65 (m, 1H), 7.29 (d, J = 10.8 Hz, |
| | | | | 1H), 7.21 (d, J = 6.0 Hz, 1H), 7.04-6.93 (m, 1H), 6.47 (s, 1H), |
| | | | | 6.19-6.12 (m, 1H), 4.62 (q, J = 7.2 Hz, 2H), 4.39-4.31 (m, |
| | | | | 2H), 3.82 (d, J = 3.2 Hz, 3H), 3.80 (s, 3H), 3.79-3.75 (m, 4H), |
| | | | | 3.66-3.58 (m, 2H), 3.24 (s, 4H), 3.09 (q, J = 6.4 Hz, 2H), 2.35 |
| | | | | (s, 1H), 2.31-2.26 (m, 1H), 2.25 (d, J = 2.8 Hz, 3H) |
| I-501 | NB | D | 648.3 | 12.16-12.06 (m, 1H), 8.21-8.00 (m, 1H), 7.73-7.61 (m, 1H), |
| | | | | 7.39 (t, J = 7.6 Hz, 1H), 7.36-7.31 (m, 1H), 7.15 (d, J = 8.0 |
| | | | | Hz, 1H), 7.04 (t, J = 7.2 Hz, 1H), 6.98-6.85 (m, 1H), 6.37 (d, |
| | | | | J = 1.6 Hz, 1H), 6.18-6.05 (m, 1H), 4.69-4.54 (m, 2H), 4.43- |
| | | | | 4.24 (m, 2H), 3.73-3.70 (m, 3H), 3.66 (d, J = 5.6 Hz, 4H), |
| | | | | 3.59 (t, J = 5.6 Hz, 2H), 3.13-3.05 (m, 2H), 2.74-2.62 (m, |
| | | | | 3H), 2.34 (s, 6H), 2.27 (d, J = 3.2 Hz, 2H) |
| I-502 | LZ | NG | 596.2 | 11.91 (br s, 1H), 8.52 (s, 1H), 8.33 (d, J = 4.4 Hz, 1H), 8.04 (s, |
| | | | | 1H), 7.65 (s, 1H), 7.42 (d, J = 4.4 Hz, 1H), 7.24 (br s, 1H), |
| | | | | 7.05 (br d, J = 4.0 Hz, 1H), 6.81 (d, J = 2.4 Hz, 1H), 6.17 (br s, |
| | | | | 1H), 5.10-4.68 (m, 4H), 4.67-4.61 (m, 2H), 4.37 (br s, 2H), |
| | | | | 3.90 (s, 3H), 3.79 (br s, 3H), 3.64 (br s, 2H), 3.11 (br s, 2H), |
| | | | | 2.35 (br d, J = 0.8 Hz, 2H) |
| I-503 | LZ | AG | 594.3 | 11.74 (br s, 1H), 7.72-7.63 (m, 1H), 7.44-7.36 (m, 3H), 7.23 |
| | | | | (s, 1H), 7.17 (br d, J = 8.4 Hz, 1H), 7.07 (br t, J = 7.6 Hz, 1H), |
| | | | | 6.94 (br d, J = 5.6 Hz, 1H), 6.73 (br s, 1H), 6.20-6.12 (m, 2H), |
| | | | | 4.92-4.68 (m, 4H), 4.42-4.33 (m, 4H), 3.77 (br s, 6H), 3.65- |
| | | | | 3.59 (m, 2H), 2.97 (br s, 2H), 2.33 (br s, 2H) |
| I-504 ^{g} | BO | MF | 350.8 | 2.23-2.41 (m, 2 H) 2.86-3.00 (m, 2 H) 3.19-3.30 (m, 6 H) |
| | | | | 3.65 (m, 2 H) 3.80 (br s, 4 H) 3.82 (s, 3 H) 3.84 (s, 3 H) 4.38 |
| | | | | (br s, 2 H) 6.15 (br d, J = 9.6 Hz, 1 H) 6.47 (d, J = 3.2 Hz, 1 H) |
| | | | | 6.93-7.06 (m, 1 H) 7.21 (d, J = 5.6 Hz, 1 H) 7.36 (m, 1 H) 7.50- |
| | | | | 7.58 (m, 1 H) 7.61-7.71 (m, 1 H) 7.79 (d, J = 2.4 Hz, 1 H) |
| | | | | 8.39-8.44 (m, 1 H) 8.50 (d, J = 5.6 Hz, 1 H) 12.10-12.32 (m, 1 H) |
| I-505 | NI | D | 609.4 | 1.36 (t, J = 7.2 Hz, 3 H) 2.24-2.39 (m, 2 H) 3.04-3.16 (m, 2 |
| | | | | H) 3.56-3.69 (m, 2 H) 3.76 (br d, J = 3.6 Hz, 3 H) 4.13 (m, 2 |
| | | | | H) 4.29-4.43 (m, 2 H) 4.55-4.70 (m, 4 H) 4.86 (br d, J = 18.4 |
| | | | | Hz, 2 H) 6.10-6.19 (m, 1 H) 6.72-6.81 (m, 1 H) 6.89-7.00 |
| | | | | (m, 1 H) 7.04-7.10 (m, 1 H) 7.17 (br d, J = 8.4 Hz, 1 H) 7.35- |
| | | | | 7.46 (m, 2 H) 7.53-7.63 (m, 1 H) 7.66-7.71 (m, 1 H) 8.08- |
| | | | | 8.13 (m, 1 H) 12.01-12.16 (m, 1 H) |
| I-506 | NP | A | 719.4 | 8.10 (s, 1H), 7.80-7.76 (m, 1H), 7.68 (s, 1H), 7.66-7.60 (m, |
| | | | | 1H), 7.49-7.43 (m, 1H), 7.37-7.33 (m, 2H), 7.15 (d, J = 8.6 |
| | | | | Hz, 1H), 7.08 (t, J = 7.2 Hz, 1H), 6.93 (d, J = 1.6 Hz, 1H), 6.76- |
| | | | | 6.58 (m, 1H), 6.05-5.95 (m, 1H), 4.63 (t, J = 6.8 Hz, 2H), |
| | | | | 4.33-4.21 (m, 1H), 4.19-4.11 (m, 1H), 3.84 (s, 3H), 3.71- |
| | | | | 3.70 (m, 3H), 3.70-3.62 (m, 5H), 3.61-3.51 (m, 3H), 3.25 (s, |
| | | | | 4H), 3.13-3.07 (m, 2H), 2.33-2.24 (m, 2H), 0.61-0.50 (m, |
| | | | | 1H), 0.10-0.01 (m, 2H), −0.26-−0.33 (m, 2H) |
| I-507 | NH | D | 609.3 | 1.28-1.38 (m, 3 H) 2.23-2.40 (m, 2 H) 3.09 (m, 2 H) 3.56- |
| | | | | 3.69 (m, 2 H) 3.78 (br d, J = 17.6 Hz, 3 H) 4.01-4.16 (m, 2 H) |
| | | | | 4.30-4.40 (m, 2 H) 4.58-4.68 (m, 3 H) 4.81 (br s, 2 H) 5.05 |
| | | | | (br s, 1 H) 6.14 (br d, J = 9.6 Hz, 1 H) 6.77 (br d, J = 17.0 Hz, 1 |
| | | | | H) 6.89-6.99 (m, 1 H) 7.07 (t, J = 7.2 Hz, 1 H) 7.17 (br d, J = 8.0 |
| | | | | Hz, 1 H) 7.24-7.31 (m, 1 H) 7.35-7.46 (m, 2 H) 7.65-7.72 |
| | | | | (m, 1 H) 8.06-8.16 (m, 1 H) 12.05-12.15 (m, 1 H) |
| I-508 ^{h} | NZ | D | 654 | 12.11 (d, J = 6.8 Hz, 1H), 11.42 (s, 1H), 8.19-8.03 (m, 1H), |
| | | | | 7.77-7.58 (m, 1H), 7.51-7.28 (m, 3H), 7.15 (d, J = 8.4 Hz, |
| | | | | 1H), 7.05 (t, J = 7.6 Hz, 1H), 6.99-6.87 (m, 1H), 6.41 (s, 1H), |
| | | | | 6.19-6.03 (m, 1H), 4.69-4.52 (m, 2H), 4.42-4.25 (m, 2H), |
| | | | | 3.81-3.73 (m, 4H), 3.73-3.70 (m, 3H), 3.68-3.65 (m, 1H), |
| | | | | 3.64 (s, 3H), 3.60 (t, J = 5.6 Hz, 1H), 3.16-3.11 (m, 2H), 3.11- |
| | | | | 3.02 (m, 4H), 2.39-2.23 (m, 2H) |
| I-509 | OK | D | 609.2 | 12.08 (d, J = 9.2 Hz, 1H), 8.15-8.06 (m, 1H), 7.74-7.60 (m, |
| | | | | 1H), 7.40 (t, J = 7.2 Hz, 2H), 7.18 (d, J = 8.0 Hz, 1H), 7.08 (t, J = |
| | | | | 7.6 Hz, 1H), 6.93 (s, 1H), 6.76 (d, J = 1.2 Hz, 1H), 6.13 (s, |
| | | | | 1H), 4.82 (d, J = 17.6 Hz, 2H), 4.67-4.57 (m, 4H), 4.40-4.30 |
| | | | | (m, 2H), 3.80-3.75 (m, 3H), 3.72 (s, 3H), 3.68-3.58 (m, 2H), |
| | | | | 3.14-3.07 (m, 2H), 2.38-2.33 (m, 2H), 2.28-2.20 (m, 3H) |
| I-510 | OC | D | 701.4 | 12.13 (d, J = 1.2 Hz, 1H), 8.23-8.04 (m, 2H), 7.74-7.63 (m, |
| | | | | 1H), 7.45-7.29 (m, 2H), 7.16 (d, J = 8.4 Hz, 1H), 7.06 (t, J = |
| | | | | 7.6 Hz, 1H), 6.91 (d, J = 6.0 Hz, 1H), 6.45 (d, J = 2.8 Hz, 1H), |
| | | | | 6.25-6.02 (m, 1H), 4.70-4.55 (m, 2H), 4.45-4.25 (m, 2H), 3.90 |
| | | | | (s, 3H), 3.82 (s, 4H), 3.73 (d, J = 3.6 Hz, 3H), 3.66 (t, J = 5.6 |
| | | | | Hz, 1H), 3.60 (t, J = 5.6 Hz, 1H), 3.36 (d, J = 7.2 Hz, 4H), |
| | | | | 3.15-3.05 (m, 2H), 2.37-2.25 (m, 2H) |
| I-511 | OI | D | 595.3 | 12.23-11.91 (m, 1H), 8.16-8.06 (m, 1H), 7.73-7.64 (m, 1H), |
| | | | | 7.46-7.30 (m, 2H), 7.15 (d, J = 7.6 Hz, 1H), 7.05 (t, J = 7.2 |
| | | | | Hz, 1H), 6.98-6.84 (m, 1H), 6.41 (s, 1H), 6.18-6.06 (m, 1H), |
| | | | | 5.54-5.29 (m, 1H), 4.68-4.56 (m, 2H), 4.43-4.27 (m, 2H), |
| | | | | 4.24-4.01 (m, 2H), 3.75-3.67 (m, 5H), 3.65-3.57 (m, 2H), |
| | | | | 3.14-3.04 (m, 2H), 2.38-2.23 (m, 2H), 2.00-1.91 (m, 2H), |
| | | | | 1.44-1.35 (m, 1H), 0.59-0.52 (m, 2H), 0.48-0.41 (m, 2H) |
| I-512 | TR | A | 633.2 | 12.11 (s, 1H), 8.10 (d, J = 9.6 Hz, 1H), 7.69 (d, J = 9.6 Hz, |
| | | | | 1H), 7.47-7.32 (m, 2H), 7.30-7.21 (m, 1H), 7.17 (d, J = 8.0 |
| | | | | Hz, 1H), 7.12-6.99 (m, 2H), 6.75 (d, J = 9.6 Hz, 1H), 5.01 (s, |
| | | | | 1H), 4.86-3.93 (m, 8H), 3.80 (s, 3H), 3.77-3.72 (m, 3H), |
| | | | | 3.71-3.56 (m, 1H), 3.43-3.40 (m, 1H), 3.15-3.11 (m, 1H), |
| | | | | 3.06-2.90 (m, 1H), 2.71-2.63 (m, 1H), 2.38-2.29 (m, 1H) |
| I-513 | TT | A | 722.3 | 12.20 (d, J = 2.8 Hz, 1H), 8.51 (d, J = 5.6 Hz, 1H), 8.41 (s, |
| | | | | 1H), 8.33 (s, 1H), 8.15-8.04 (m, 1H), 7.79 (d, J = 2.4 Hz, 1H), |
| | | | | 7.69 (d, J = 8.4 Hz, 1H), 7.36 (dd, J = 2.4, 10.4 Hz, 1H), 7.21 |
| | | | | (d, J = 6.0 Hz, 1H), 7.11 (d, J = 6.0 Hz, 1H), 6.47 (s, 1H), 4.62 |
| | | | | (q, J = 7.2 Hz, 3H), 4.33-3.97 (m, 1H), 3.91-3.72 (m, 11H), |
| | | | | 3.69-3.53 (m, 1H), 3.41 (d, J = 10.0 Hz, 1H), 3.25 (s, 4H), |
| | | | | 3.12 (t, J = 7.2 Hz, 1H), 3.05-2.89 (m, 1H), 2.69-2.60 (m, |
| | | | | 1H), 2.41-2.30 (m, 1H) |
| I-514 ^{f} | AW | PE | 691.4 | 9.36 (s, 1H), 8.41 (s, 1H), 7.54-7.45 (m, 2H), 7.02-6.95 (m, |
| | | | | 1H), 6.89-6.81 (m, 1H), 6.71 (d, J = 7.2 Hz, 1H), 6.43-6.33 |
| | | | | (m, 1H), 6.24-6.18 (m, 1H), 6.16 (s, 1H), 4.56-4.52 (m, 2H), |
| | | | | 4.48 (s, 1H), 4.28 (s, 1H), 4.02-3.93 (m, 4H), 3.85 (s, 3H), |
| | | | | 3.78 (t, J = 4.8 Hz, 1H), 3.57 (t, J = 5.6 Hz, 1H), 3.51-3.43 |
| | | | | (m, 4H), 3.04-2.98 (m, 2H), 2.86-2.62 (m, 3H), 2.60-2.50 |
| | | | | (m, 2H), 2.38 (s, 3H), 2.35 (d, J = 3.2 Hz, 2H), 1.10 (d, J = 4.4 |
| | | | | Hz, 3H) |
| I-515 | PG | PE | 690.5 | 12.30 (S, 1H), 8.27 (s, 1H), 7.73-7.68 (m, 1H), 7.45-7.33 (m, |
| | | | | 1H), 7.28 (s, 1H), 6.83 (s, 1H), 6.83-6.80 (m, 1H), 6.78-6.74 |
| | | | | (m, 1H), 6.69 (s, 1H), 6.35 (d, J = 2.4 Hz, 1H), 6.21-6.16 (m, |
| | | | | 1H), 6.14 (s, 1H), 4.36 (s, 3H), 4.35-4.24 (m, 2H), 3.77 (s, |
| | | | | 4H), 3.74 (s, 3H), 3.65-3.56 (m, 2H), 3.00-2.93 (m, 6H), |
| | | | | 2.48-2.41 (m, 4H), 2.35-2.26 (m, 2H), 2.20 (s, 3H), 0.99 (t, J = |
| | | | | 7.6 Hz, 3H) |
| I-516 | RS | D | 609.4 | 12.17 (s, 1H), 8.18-8.04 (m, 1H), 7.77-7.65 (m, 1H), 7.46- |
| | | | | 7.33 (m, 2H), 7.23 (s, 1H), 7.16 (d, J = 8.0 Hz, 1H), 7.05 (t, J = |
| | | | | 7.6 Hz, 1H), 7.00-6.88 (m, 1H), 6.49 (s, 1H), 6.22-5.99 (m, |
| | | | | 1H), 4.81 (s, 2H), 4.69-4.56 (m, 2H), 4.43-4.28 (m, 2H), |
| | | | | 3.86-3.79(m, 2H), 3.77-3.72 (m, 4H), 3.71-3.67 (m, 2H), |
| | | | | 3.67-3.63 (m, 1H), 3.60 (br t, J = 5.6 Hz, 1H), 3.09 (q, J = 6.8 |
| | | | | Hz, 2H), 2.60 (t, J = 4.8 Hz, 2H), 2.37-2.24 (m, 2H) |
| I-517 | PB | A | 732.5 | 8.13-8.06 (m, 1H), 7.78 (d, J = 2.4 Hz, 1H), 7.73-7.62 (m, |
| | | | | 3H), 7.36 (dd, J = 2.4, 10.4 Hz, 1H), 7.21 (d, J = 8.0 Hz, 1H), |
| | | | | 6.91 (d, J = 1.6 Hz, 1H), 6.82-6.62 (m, 1H), 6.05-5.93 (m, |
| | | | | 1H), 4.62 (t, J = 6.8 Hz, 2H), 4.39-4.27 (m, 1H), 4.16-4.03 |
| | | | | (m, 1H), 3.83 (s, 3H), 3.69-3.60 (m, 4H), 3.59-3.49 (m, 2H), |
| | | | | 3.45-3.36 (m, 2H), 3.24 (d, J = 0.8 Hz, 4H), 3.09 (t, J = 6.8 |
| | | | | Hz, 2H), 2.55 (s, 3H), 2.42 (d, J = 7.6 Hz, 2H), 2.31 (d, J = 4.0 |
| | | | | Hz, 2H), 1.03 (t, J = 7.2 Hz, 3H), 0.55-0.41 (m, 1H), 0.14- |
| | | | | 0.05 (m, 2H), −0.24 (d, J = 5.2 Hz, 2H) |
| I-518 | RR | D | 609.3 | 12.13 (s, 1H), 8.18-8.05 (m, 1H), 7.74-7.63 (m, 1H), 7.47- |
| | | | | 7.31 (m, 2H), 7.27-7.19 (m, 1H), 7.16 (d, J = 8.4 Hz, 1H), |
| | | | | 7.05 (t, J = 7.2 Hz, 1H), 6.99-6.87 (m, 1H), 6.52-6.42 (m, |
| | | | | 1H), 6.25-6.01 (m, 1H), 4.74-4.56 (m, 4H), 4.41-4.26 (m, |
| | | | | 2H), 3.96-3.85 (m, 2H), 3.77-3.74 (m, 3H), 3.67 (s, 3H), |
| | | | | 3.65-3.57 (m, 2H), 3.14-3.04 (m, 2H), 2.83-2.72 (m, 2H), |
| | | | | 2.39-2.25 (m, 2H) |
| I-519 | MA | PI | 679.2 | 12.31 (br s, 1H), 8.27 (s, 1H), 8.14-8.06 (m, 1H), 7.76 (d, J = |
| | | | | 2.8 Hz, 1H), 7.72-7.66 (m, 1H), 7.64 (d, J = 2.8 Hz, 1H), 7.28 |
| | | | | (s, 1H), 6.97-6.79 (m, 1H), 6.37 (d, J = 2.0 Hz, 1H), 6.20- |
| | | | | 6.10 (m, 1H), 4.62 (q, J = 7.2 Hz, 2H), 4.41-4.29 (m, 2H), |
| | | | | 3.90 (s, 3H), 3.77 (br s, 4H), 3.67-3.57 (m, 2H), 3.48 (br s, |
| | | | | 4H), 3.09 (q, J = 6.4 Hz, 2H), 2.52-2.51 (m, 3H), 2.48-2.42 |
| | | | | (m, 2H), 2.36-2.25 (m, 2H), 0.99 (t, J = 7.6 Hz, 3H) |
| I-520 | PJ | PI | 693.3 | 12.30 (br s, 1H), 8.27 (s, 1H), 8.15-8.06 (m, 1H), 7.71-7.65 |
| | | | | (m, 1H), 7.63 (s, 1H), 7.28 (s, 1H), 6.96-6.79 (m, 1H), 6.35 |
| | | | | (br s, 1H), 6.16 (br d, J = 13.2 Hz, 1H), 4.62 (q, J = 7.2 Hz, |
| | | | | 2H), 4.40-4.28 (m, 2H), 3.89 (s, 3H), 3.76 (br s, 4H), 3.67- |
| | | | | 3.57 (m, 2H), 3.37 (br s, 4H), 3.09 (q, J = 6.4 Hz, 2H), 2.54- |
| | | | | 2.51 (m, 3H), 2.48-2.42 (m, 2H), 2.34 (br d, J = 3.6 Hz, 2H), |
| | | | | 2.28 (s, 3H), 0.99 (t, J = 7.6 Hz, 3H) |
| I-521 | PL | A | 721.3 | 8.11 (d, J = 2.4 Hz, 1H), 7.78 (d, J = 2.4 Hz, 1H), 7.70-7.62 |
| | | | | (m, 2H), 7.46 (t, J = 7.6 Hz, 1H), 7.38-7.33 (m, 2H), 7.16 (d, J = |
| | | | | 8.4 Hz, 1H), 7.09 (t, J = 7.6 Hz, 1H), 6.94 (s, 1H), 6.73-6.55 |
| | | | | (m, 1H), 6.02-5.94 (m, 1H), 4.63 (t, J = 6.8 Hz, 2H), 4.35- |
| | | | | 4.24 (m, 1H), 4.10 (d, J = 17.2 Hz, 1H), 3.84 (s, 3H), 3.72 (s, |
| | | | | 3H), 3.68 (s, 4H), 3.62-3.58 (m, 1H), 3.56-3.53 (m, 1H), |
| | | | | 3.51-3.41 (m, 2H), 3.24 (s, 4H), 3.14-3.08 (m, 2H), 2.35- |
| | | | | 2.27 (m, 2H), 1.37 (d, J = 6.4 Hz, 1H), 0.25-0.20 (m, 3H), |
| | | | | 0.15 (t, J = 6.4 Hz, 3H) |
| I-522 | PB | SC | 731.7 | 7.78 (d, J = 2.0 Hz, 1H), 7.71-7.66 (m, 3H), 7.41 (br s, 1H), |
| | | | | 7.35 (dd, J = 2.4, 10.4 Hz, 1H), 7.21 (br d, J = 7.6 Hz, 1H), |
| | | | | 6.91 (s, 1H), 6.80-6.65 (m, 1H), 6.19 (s, 1H), 6.02-5.95 (m, |
| | | | | 1H), 4.38-4.34 (m, 2H), 4.11-4.04 (m, 1H), 3.83 (s, 3H), |
| | | | | 3.66 (br s, 4H), 3.57 (br s, 1H), 3.53-3.50 (m, 1H), 3.41 (br d, |
| | | | | J = 4.8 Hz, 1H), 3.24 (br s, 4H), 2.97 (br t, J = 6.4 Hz, 2H), |
| | | | | 2.55 (s, 3H), 2.47-2.38 (m, 4H), 2.26 (br d, J = 14.4 Hz, 2H), |
| | | | | 1.03 (t, J = 7.2 Hz, 3H), 0.51-0.44 (m, 1H), 0.08 (br dd, J = |
| | | | | 4.4, 8.0 Hz, 2H), −0.21-−0.27 (m, 2H) |
| I-523 | PB | SB | 748.7 | 7.78 (d, J = 2.4 Hz, 1H), 7.70-7.66 (m, 3H), 7.54 (d, J = 3.2 |
| | | | | Hz, 1H), 7.36 (dd, J = 2.4, 10.4 Hz, 1H), 7.22 (d, J = 7.6 Hz, |
| | | | | 1H), 6.92 (s, 1H), 6.81-6.65 (m, 1H), 6.05-5.97 (m, 1H), |
| | | | | 4.37 (br d, J = 17.6 Hz, 1H), 4.16-4.07 (m, 1H), 3.84 (s, 3H), |
| | | | | 3.66 (br d, J = 4.4 Hz, 4H), 3.58 (br d, J = 4.0 Hz, 1H), 3.54- |
| | | | | 3.48 (m, 1H), 3.41 (br d, J = 6.4 Hz, 1H), 3.25 (br s, 4H), 2.95- |
| | | | | 2.89 (m, 2H), 2.55 (s, 3H), 2.47-2.38 (m, 4H), 2.33 (br s, 1H), |
| | | | | 2.29-2.22 (m, 1H), 1.03 (t, J = 7.6 Hz, 3H), 0.53-0.46 (m, |
| | | | | 1H), 0.08 (br dd, J = 5.6, 7.6 Hz, 2H), −0.20-−0.26 (m, 2H) |
| I-524 | PA | A | 734.4 | 8.14-8.05 (m, 1H), 7.78 (d, J = 2.4 Hz, 1H), 7.72-7.64 (m, |
| | | | | 3H), 7.36 (dd, J = 2.4, 10.4 Hz, 1H), 7.22 (d, J = 8.0 Hz, 1H), |
| | | | | 6.92 (d, J = 1.6 Hz, 1H), 6.78 (s, 1H), 6.62 (s, 1H), 6.07-5.94 |
| | | | | (m, 1H), 4.63 (t, J = 6.8 Hz, 2H), 4.42-4.28 (m, 1H), 4.10- |
| | | | | 4.00 (m, 1H), 3.83 (s, 3H), 3.72-3.59 (m, 5H), 3.56-3.52 (m, |
| | | | | 1H), 3.23 (br s, 5H), 3.15-3.06 (m, 2H), 2.54 (s, 3H), 2.46 (br |
| | | | | d, J = 3.6 Hz, 2H), 2.43-2.38 (m, 1H), 2.28 (br d, J = 10.4 Hz, |
| | | | | 1H), 1.38-1.28 (m, 1H), 1.02 (t, J = 7.6 Hz, 3H), 0.27-0.18 |
| | | | | (m, 6H) |
| I-525 | BO | PP | 712.4 | 12.29 (s, 1H), 8.27 (s, 1H), 7.78 (d, J = 2.4 Hz, 1H), 7.68-7.61 |
| | | | | (m, 1H), 7.56-7.50 (m, 1H), 7.35 (dd, J = 2.4, 10.4 Hz, 1H), |
| | | | | 7.28 (s, 1H), 6.96-6.81 (m, 1H), 6.35 (d, J = 2.4 Hz, 1H), 6.20- |
| | | | | 6.11 (m, 1H), 4.37 (s, 2H), 3.83 (s, 3H), 3.76 (s, 4H), 3.67- |
| | | | | 3.61 (m, 2H), 3.24 (d, J = 6.8 Hz, 6H), 2.95-2.88 (m, 2H), |
| | | | | 2.47-2.42 (m, 4H), 2.36 (s, 2H), 2.27 (d, J = 3.6 Hz, 1H), 0.99 |
| | | | | (t, J = 7.6 Hz, 3H) |
| I-526 | AW | PP | 708.4 | 12.29 (s, 1H), 8.28 (s, 1H), 7.71-7.60 (m, 1H), 7.57-7.49 (m, |
| | | | | 1H), 7.28 (s, 1H), 7.15 (d, J = 8.0 Hz, 1H), 6.96-6.81 (m, 1H), |
| | | | | 6.73 (d, J = 8.0 Hz, 1H), 6.36 (d, J = 3.2 Hz, 1H), 6.21-6.11 |
| | | | | (m, 1H), 4.38 (s, 2H), 3.82-3.75 (m, 4H), 3.75 (s, 3H), 3.67- |
| | | | | 3.61 (m, 2H), 3.31-3.26 (m, 4H), 3.26-3.20 (m, 2H), 2.92 (q, |
| | | | | J = 7.2 Hz, 2H), 2.51 (s, 3H), 2.48-2.42 (m, 2H), 2.42-2.29 |
| | | | | (m, 2H), 2.27 (s, 3H), 0.99 (t, J = 7.6 Hz, 3H) |
| I-527 | PB | PV | 709.3 | 13.23-11.13 (m, 1H), 7.78 (d, J = 2.4 Hz, 1H), 7.71-7.66 (m, |
| | | | | 2H), 7.36 (dd, J = 2.4, 10.4 Hz, 1H), 7.21 (d, J = 7.6 Hz, 1H), |
| | | | | 6.91 (s, 1H), 6.82-6.63 (m, 1H), 6.05-5.96 (m, 1H), 4.35 (d, J = |
| | | | | 17.2 Hz, 1H), 4.16-4.04 (m, 1H), 3.83 (s, 3H), 3.75-3.70 |
| | | | | (m, 1H), 3.65 (d, J = 6.4 Hz, 3H), 3.60-3.52 (m, 2H), 3.49- |
| | | | | 3.38 (m, 2H), 3.24 (s, 4H), 2.70-2.60 (m, 2H), 2.55 (s, 3H), |
| | | | | 2.47-2.42 (m, 4H), 2.36-2.23 (m, 2H), 1.03 (t, J = 7.6 Hz, |
| | | | | 3H), 0.54-0.44 (m, 1H), 0.15-0.06 (m, 2H), −0.18-−0.26 (m, 2H) |
| I-528 | PJ | PP | 709.3 | 12.30 (s, 1H), 8.27 (s, 1H), 7.70-7.61 (m, 2H), 7.57-7.50 (m, |
| | | | | 1H), 7.28 (s, 1H), 6.96-6.80 (m, 1H), 6.36 (d, J = 3.2 Hz, 1H), |
| | | | | 6.21-6.09 (m, 1H), 4.38 (s, 2H), 3.89 (s, 3H), 3.77 (s, 4H), |
| | | | | 3.67-3.61 (m, 2H), 3.37 (s, 3H), 3.37-3.34 (m, 2H), 3.24 (q, J = |
| | | | | 7.2 Hz, 2H), 2.92 (q, J = 7.2 Hz, 2H), 2.49-2.41 (m, 4H), |
| | | | | 2.35 (d, J = 14.4 Hz, 2H), 2.28 (s, 3H), 0.99 (t, J = 7.6 Hz, 3H) |
| I-529 | NH | PI | 622.4 | 12.30 (s, 1H), 8.35-8.21 (m, 1H), 8.14-8.05 (m, 1H), 7.83- |
| | | | | 7.51 (m, 1H), 7.33-7.20 (m, 2H), 6.97-6.82 (m, 1H), 6.71- |
| | | | | 6.62 (m, 1H), 6.21-6.12 (m, 1H), 5.00 (s, 1H), 4.79 (s, 3H), |
| | | | | 4.64-4.48 (m, 3H), 4.40-4.30 (m, 2H), 4.15-4.00 (m, 2H), |
| | | | | 3.66-3.57 (m, 2H), 3.12-3.05 (m, 2H), 2.53 (s, 3H), 2.46- |
| | | | | 2.22 (m, 3H), 1.38-1.27 (m, 3H), 1.08-0.97 (m, 3H) |
| I-530 | SJ | A | 645.3 | 8.10 (s, 1H), 7.86-7.76 (m, 1H), 7.69 (s, 1H), 7.46 (t, J = 7.6 |
| | | | | Hz, 1H), 7.35 (d, J = 7.2 Hz, 1H), 7.29-7.13 (m, 2H), 7.11- |
| | | | | 7.04 (m, 2H), 6.79-6.58 (m, 1H), 6.06-5.94 (m, 1H), 4.79 (d, |
| | | | | J = 12.8 Hz, 2H), 4.63 (t, J = 6.8 Hz, 2H), 4.58 (s, 2H), 4.28 (t, |
| | | | | J = 18.0 Hz, 1H), 4.18-3.96 (m, 3H), 3.71 (s, 3H), 3.69-3.52 |
| | | | | (m, 4H), 3.14-3.06 (m, 2H), 2.35-2.23 (m, 2H), 1.37-1.24 |
| | | | | (m, 3H), 0.55 (d, J = 5.6 Hz, 1H), 0.13-0.01 (m, 2H), −0.29 (s, 2H) |
| I-531 | LZ | PI | 608.2 | 12.31 (s, 1H), 8.35-8.23 (m, 1H), 8.14-8.07 (m, 1H), 7.76- |
| | | | | 7.62 (m, 1H), 7.36-7.19 (m, 2H), 6.97-6.81 (m, 1H), 6.66 (s, |
| | | | | 1H), 6.16 (s, 1H), 4.96 (s, 1H), 4.78 (d, J = 13.6 Hz, 3H), 4.62 |
| | | | | (d, J = 7.6 Hz, 3H), 4.38 (s, 2H), 3.82-3.70 (m, 3H), 3.66- |
| | | | | 3.57 (m, 2H), 3.13-3.06 (m, 2H), 2.53 (s, 3H), 2.35 (d, J = 2.8 |
| | | | | Hz, 3H), 1.06-0.96 (m, 3H) |
| I-532 | BO | RD | 627.2 | 13.30-11.44 (m, 1H), 8.47 (s, 1H), 8.17-8.07 (m, 1H), 7.81 |
| | | | | (d, J = 2.4 Hz, 1H), 7.73-7.65 (m, 1H), 7.41-7.37 (m, 1H), |
| | | | | 7.32-7.27 (m, 1H), 6.95-6.91 (m, 1H), 6.17-6.11 (m, 1H), |
| | | | | 4.63 (q, J = 7.2 Hz, 2H), 4.37-4.30 (m, 2H), 3.86 (s, 3H), 3.83 |
| | | | | (br s, 4H), 3.66 (br t, J = 6.0 Hz, 1H), 3.61 (s, 1H), 3.32-3.29 |
| | | | | (m, 4H), 3.13-3.07 (m, 2H), 2.38-2.28 (m, 2H) |
| I-533 | SA | A | 732.3 | 8.40 (d, J = 2.8 Hz, 1H), 8.11-8.09 (m, 1H), 7.78 (d, J = 2.4 |
| | | | | Hz, 1H), 7.71-7.68 (m, 2H), 7.35 (dd, J = 2.4, 10.4 Hz, 1H), |
| | | | | 7.30 (s, 1H), 6.93 (d, J = 1.6 Hz, 1H), 6.82-6.65 (m, 1H), 6.03- |
| | | | | 5.96 (m, 1H), 4.64-4.60 (m, 2H), 4.39-4.29 (m, 1H), 4.12- |
| | | | | 4.05 (m, 1H), 3.84 (s, 3H), 3.69-3.62 (m, 4H), 3.55-3.51 (m, |
| | | | | 1H), 3.44 (br dd, J = 3.2, 4.4 Hz, 1H), 3.25 (br d, J = 3.6 Hz, |
| | | | | 4H), 3.09 (br t, J = 6.8 Hz, 2H), 2.55 (s, 3H), 2.40-2.21 (m, |
| | | | | 6H), 0.99 (t, J = 7.6 Hz, 3H), 0.52-0.44 (m, 1H), 0.12-0.06 |
| | | | | (m, 2H), −0.22-−0.30 (m, 2H) |
| I-534 | SG | A | 731.4 | 8.10 (s, 1H), 7.75-7.58 (m, 3H), 7.21 (d, J = 7.6 Hz, 1H), 6.93- |
| | | | | 6.84 (m, 3H), 6.81-6.62 (m, 2H), 6.05-5.95 (m, 1H), 4.62 |
| | | | | (t, J = 6.8 Hz, 2H), 4.41-4.27 (m, 1H), 4.09 (d, J = 17.6 Hz, |
| | | | | 1H), 3.79 (s, 3H), 3.78-3.64 (m, 4H), 3.60-3.50 (m, 2H), |
| | | | | 3.44-3.39 (m, 1H), 3.13-3.04 (m, 2H), 2.98-2.85 (m, 4H), |
| | | | | 2.55 (s, 3H), 2.46-2.34 (m, 3H), 2.33-2.24 (m, 2H), 1.03 (t, J = |
| | | | | 7.6 Hz, 3H), 0.54-0.41 (m, 1H), 0.13-0.02 (m, 2H), −0.19-−0.31 |
| | | | | (m, 2H) |
| I-535 | SM | A | 658.3 | 8.15-8.01 (m, 1H), 7.89-7.81 (m, 1H), 7.71-7.60 (m, 2H), |
| | | | | 7.31-7.14 (m, 2H), 7.08 (d, J = 2.4 Hz, 1H), 6.84-6.62 (m, |
| | | | | 1H), 6.05-5.96 (m, 1H), 4.77 (s, 2H), 4.63 (t, J = 6.8 Hz, 2H), |
| | | | | 4.58 (s, 2H), 4.34 (t, J = 16.4 Hz, 1H), 4.14-4.06 (m, 2H), |
| | | | | 3.99 (q, J = 6.8 Hz, 1H), 3.63-3.52 (m, 2H), 3.42 (s, 1H), 3.17- |
| | | | | 3.05 (m, 2H), 2.55 (s, 3H), 2.39 (s, 3H), 2.34-2.23 (m, 2H), |
| | | | | 1.38-1.23 (m, 3H), 1.04 (q, J = 6.8 Hz, 3H), 0.55-0.43 (m, |
| | | | | 1H), 0.10 (d, J = 6.0 Hz, 2H), −0.24 (s, 2H) |
| I-536 | P | PI | 611.4 | 12.29 (s, 1H), 8.26 (s, 1H), 8.15-8.06 (m, 1H), 7.80-7.43 (m, |
| | | | | 1H), 7.28 (s, 1H), 6.97-6.80 (m, 1H), 6.27 (d, J = 2.8 Hz, 1H), |
| | | | | 6.22-6.09 (m, 1H), 4.61 (q, J = 7.2 Hz, 2H), 4.41-4.30 (m, |
| | | | | 2H), 3.64 (t, J = 5.6 Hz, 1H), 3.58 (d, J = 5.2 Hz, 5H), 3.32- |
| | | | | 3.26 (m, 2H), 3.09 (q, J = 6.8 Hz, 2H), 2.57-2.52 (m, 4H), |
| | | | | 2.48-2.40 (m, 3H), 2.34 (d, J = 3.2 Hz, 1H), 2.26 (d, J = 3.2 |
| | | | | Hz, 1H), 1.63 (d, J = 3.2 Hz, 1H), 0.99 (t, J = 7.6 Hz, 3H), 0.46- |
| | | | | 0.37 (m, 2H), 0.35-0.29 (m, 2H) |
| I-537 | NH | PE | 621.3 | 12.43-12.13 (m, 1H), 8.43-8.21 (m, 1H), 7.79-7.65 (m, 1H), |
| | | | | 7.45-7.33 (m, 1H), 7.33-7.20 (m, 2H), 6.94-6.81 (m, 1H), |
| | | | | 6.73-6.61 (m, 1H), 6.18 (br d, J = 14.4 Hz, 2H), 5.02-4.58 |
| | | | | (m, 4H), 4.40-4.29 (m, 4H), 4.14-3.98 (m, 2H), 3.67-3.54 |
| | | | | (m, 2H), 2.97 (q, J = 6.8 Hz, 2H), 2.53 (d, J = 4.4 Hz, 5H), 2.34- |
| | | | | 2.25 (m, 2H), 1.37-1.25 (m, 3H), 1.06-0.98 (m, 3H) |
| I-538 | NH | PP | 638.2 | 12.34-12.26 (m, 1H), 8.34-8.26 (m, 1H), 7.70-7.59 (m, 1H), |
| | | | | 7.59-7.48 (m, 1H), 7.30 (s, 1H), 7.29-7.22 (m, 1H), 6.96- |
| | | | | 6.83 (m, 1H), 6.72-6.62 (m, 1H), 6.21-6.12 (m, 1H), 5.07- |
| | | | | 4.93 (m, 1H), 4.79 (s, 2H), 4.59 (s, 1H), 4.38 (s, 2H), 4.14- |
| | | | | 4.08 (m, 1H), 4.03 (q, J = 7.2 Hz, 1H), 3.67-3.62 (m, 2H), |
| | | | | 3.27-3.21 (m, 2H), 2.95-2.89 (m, 2H), 2.53 (s, 3H), 2.48- |
| | | | | 2.44 (m, 2H), 2.38-2.25 (m, 2H), 1.37-1.28 (m, 3H), 1.02 |
| | | | | (td, J = 7.6, 14.8 Hz, 3H) |
| I-539 ⁱ | PB | QS | 709.5 | 7.78 (d, J = 2.4 Hz, 1H), 7.68 (br d, J = 7.2 Hz, 2H), 7.35 (dd, J = |
| | | | | 2.4, 10.4 Hz, 1H), 7.21 (d, J = 8.0 Hz, 1H), 6.91 (s, 1H), 6.80- |
| | | | | 6.63 (m, 1H), 6.03-5.95 (m, 1H), 4.52-4.02 (m, 2H), 3.83 |
| | | | | (s, 3H), 3.81-3.72 (m, 2H), 3.70-3.54 (m, 6H), 3.24 (br s, |
| | | | | 5H), 2.55 (s, 3H), 2.40-2.22 (m, 3H), 1.24-1.13 (m, 4H), |
| | | | | 1.03 (t, J = 7.5 Hz, 3H), 0.54-0.43 (m, 1H), 0.09 (br s, |
| | | | | 2H), −0.18-−0.27 (m, 2H) |
| I-540 ^{h} | OB | BM | 653.3 | 12.32-12.11 (m, 1H), 11.85-11.63 (m, 1H), 8.50 (d, J = 5.6 |
| | | | | Hz, 1H), 8.41 (s, 1H), 8.35-8.30 (m, 1H), 8.16-8.05 (m, 1H), |
| | | | | 7.72-7.65 (m, 1H), 7.33 (s, 1H), 7.21 (d, J = 6.0 Hz, 1H), 7.07- |
| | | | | 6.91 (m, 1H), 6.45 (br s, 1H), 6.15 (br d, J = 11.2 Hz, 1H), |
| | | | | 4.67-4.58 (m, 2H), 4.41-4.30 (m, 2H), 3.82 (d, J = 2.8 Hz, |
| | | | | 3H), 3.77 (br s, 4H), 3.65-3.59 (m, 2H), 3.12-3.07 (m, 2H), |
| | | | | 3.00 (br s, 4H), 2.38-2.26 (m, 4H), 1.13 (t, J = 7.6 Hz, 3H) |
| I-541 | OJ | D | 595.5 | 12.09 (d, J = 7.2 Hz, 1H), 8.13-8.08 (m, 1H), 7.71-7.66 (m, |
| | | | | 1H), 7.55 (s, 1H), 7.43-7.37 (m, 2H), 7.17 (d, J = 8.0 Hz, |
| | | | | 1H), 7.09-7.04 (m, 1H), 6.99-6.90 (m, 1H), 6.76 (d, J = 13.2 |
| | | | | Hz, 1H), 6.17-6.11 (m, 1H), 4.95 (s, 1H), 4.79 (s, 1H), 4.73 |
| | | | | (s, 1H), 4.65-4.60 (m, 2H), 4.56 (s, 1H), 4.39-4.32 (m, 2H), |
| | | | | 3.79 (d, J = 2.8 Hz, 1H), 3.76-3.75 (m, 1H), 3.67 (s, 3H), 3.60 |
| | | | | (s, 3H), 3.12-3.07 (m, 2H), 2.36-2.26 (m, 2H) |
| I-542 | RF | A | 718.5 | 8.61 (s, 1H), 8.53 (d, J = 5.2 Hz, 1H), 8.18-7.91 (m, 1H), 7.82- |
| | | | | 7.61 (m, 3H), 7.44 (d, J = 4.4 Hz, 1H), 7.39-7.31 (m, 1H), |
| | | | | 6.98-6.89 (m, 1H), 6.86-6.64 (m, 1H), 6.06-5.95 (m, 1H), |
| | | | | 4.62 (t, J = 6.4 Hz, 2H), 4.41-4.28 (m, 1H), 4.08 (d, J = 18.4 |
| | | | | Hz, 1H), 3.83 (s, 3H), 3.65 (dd, J = 2.0, 3.2 Hz, 2H), 3.57- |
| | | | | 3.50 (m, 2H), 3.48-3.39 (m, 4H), 3.25 (s, 4H), 3.09 (t, J = 6.4 |
| | | | | Hz, 2H), 2.34-2.18 (m, 4H), 1.00-0.90 (m, 3H), 0.56-0.42 |
| | | | | (m, 1H), 0.16-0.04 (m, 2H), −0.15-−0.29 (m, 2H) |
| I-543 | BO | PZ | 696.4 | 8.24 (s, 1H), 8.11-8.05 (m, 1H), 7.78 (br s, 1H), 7.67 (s, 1H), |
| | | | | 7.58 (br d, J = 7.6 Hz, 1H), 7.39-7.34 (m, 2H), 7.21-7.16 (m, |
| | | | | 1H), 6.97-6.91 (m, 1H), 4.62-4.57 (m, 2H), 4.40-4.34 (m, |
| | | | | 2H), 3.84 (s, 4H), 3.64-3.54 (m, 4H), 3.11-2.98 (m, 3H), |
| | | | | 2.55 (br s, 5H), 2.45-2.31 (m, 6H), 1.06 (br t, J = 7.6 Hz, 3H) |
| I-544 | TB | A | 633.3 | 8.11 (s, 1H), 8.03-7.89 (m, 1H), 7.80-7.51 (m, 2H), 7.38- |
| | | | | 7.13 (m, 2H), 6.98-6.63 (m, 1H), 6.49 (d, J = 1.6 Hz, 1H), |
| | | | | 6.17-5.94 (m, 1H), 4.78 (s, 2H), 4.63 (t, J = 6.8 Hz, 2H), 4.59 |
| | | | | (s, 2H), 4.35 (s, 1H), 4.21-4.08 (m, 2H), 3.99 (d, J = 7.2 Hz, |
| | | | | 1H), 3.96-3.67 (m, 4H), 3.59 (d, J = 17.6 Hz, 2H), 3.14-3.01 |
| | | | | (m, 2H), 2.38-2.26 (m, 2H), 1.37 (s, 6H), 0.58 (d, J = 6.8 Hz, |
| | | | | 1H), 0.15 (s, 2H), −0.08-−0.27 (m, 2H) |
| I-545 | QV | QT | 721.4 | 12.94-11.95 (m, 1H), 7.78 (d, J = 2.0 Hz, 1H), 7.75-7.66 (m, |
| | | | | 2H), 7.40-7.31 (m, 1H), 7.22 (d, J = 7.6 Hz, 1H), 6.94 (s, |
| | | | | 1H), 6.81-6.62 (m, 1H), 6.06 (d, J = 16.4 Hz, 1H), 4.54-4.07 |
| | | | | (m, 2H), 3.83 (s, 3H), 3.67 (s, 3H), 3.66-3.58 (m, 3H), 3.38 |
| | | | | (d, J = 5.6 Hz, 1H), 3.25 (s, 4H), 2.85-2.76 (m, 2H), 2.46- |
| | | | | 2.43 (m, 1H), 2.39 (s, 2H), 2.26 (d, J = 2.4 Hz, 1H), 2.20 (s, |
| | | | | 3H), 1.96-1.82 (m, 1H), 1.30-1.26 (m, 3H), 1.20 (d, J = 6.4 |
| | | | | Hz, 2H), 0.54-0.42 (m, 1H), 0.09 (d, J = 6.0 Hz, 2H), −0.21-−0.35 |
| | | | | (m, 2H) |
| I-546 | RJ | A | 746.5 | 8.10 (d, J = 2.4 Hz, 1H), 7.71-7.65 (m, 3H), 7.29 (d, J = 10.4 |
| | | | | Hz, 1H), 7.21 (d, J = 7.8 Hz, 1H), 6.91 (d, J = 1.6 Hz, 1H), 6.82- |
| | | | | 6.62 (m, 1H), 6.04-5.93 (m, 1H), 4.62 (t, J = 6.4 Hz, 2H), |
| | | | | 4.40-4.28 (m, 1H), 4.09 (d, J = 16.8 Hz, 1H), 3.80 (s, 3H), |
| | | | | 3.72-3.49 (m, 7H), 3.45-3.35 (m, 2H), 3.23 (s, 3H), 3.14- |
| | | | | 3.06 (m, 2H), 2.55 (s, 3H), 2.47-2.40 (m, 2H), 2.32 (dd, J = |
| | | | | 2.4, 4.0 Hz, 2H), 2.25 (d, J = 2.8 Hz, 3H), 1.03 (t, J = 7.6 Hz, |
| | | | | 3H), 0.55-0.46 (m, 1H), 0.12-0.05 (m, 2H), −0.24 (td, J = 5.2, |
| | | | | 9.6 Hz, 2H) |
| I-547 | NH | TX | 586.3 | 12.32-11.83 (m, 1H), 8.23-8.00 (m, 1H), 7.83-7.57 (m, 1H), |
| | | | | 7.44-7.35 (m, 1H), 7.35-7.25 (m, 1H), 6.94-6.85 (m, 1H), |
| | | | | 6.14-6.01 (m, 1H), 5.19-5.06 (m, 1H), 4.99-4.90 (m, 1H), |
| | | | | 4.89-4.79 (m, 1H), 4.71-4.56 (m, 3H), 4.39-4.26 (m, 2H), |
| | | | | 4.19-4.07 (m, 2H), 3.83-3.72 (m, 1H), 3.68-3.55 (m, 2H), |
| | | | | 3.14-3.03 (m, 2H), 2.94-2.85 (m, 1H), 2.79-2.60 (m, 4H), |
| | | | | 2.37 (s, 5H), 2.02-1.85 (m, 1H), 1.39 (t, J = 7.2 Hz, 3H) |
| I-548 | SZ | A | 665.4 | 8.13-8.06 (m, 1H), 7.72-7.64 (m, 2H), 7.60 (d, J = 1.6 Hz, |
| | | | | 1H), 7.22 (d, J = 8.0 Hz, 1H), 6.95 (d, J = 1.2 Hz, 1H), 6.14- |
| | | | | 6.02 (m, 1H), 4.65-4.58 (m, 2H), 4.44-4.24 (m, 1H), 4.17- |
| | | | | 4.05 (m, 1H), 3.73-3.61 (m, 1H), 3.58-3.38 (m, 7H), 3.35- |
| | | | | 3.28 (m, 4H), 3.12-3.02 (m, 2H), 2.55 (s, 3H), 2.47-2.39 (m, |
| | | | | 2H), 2.37-2.26 (m, 2H), 1.70-1.61 (m, 1H), 1.03 (t, J = 7.2 |
| | | | | Hz, 3H), 0.49-0.39 (m, 3H), 0.36-0.29 (m, 2H), 0.18-0.07 |
| | | | | (m, 2H), −0.19-−0.28 (m, 2H) |
| I-549 | QR | A | 676.3 | 8.33 (d, J = 3.2 Hz, 1H), 7.98-7.85 (m, 2H), 7.56-7.36 (m, |
| | | | | 2H), 7.22-6.90 (m, 2H), 6.28 (br d, J = 13.6 Hz, 1H), 4.99 (s, |
| | | | | 1H), 4.90-4.76 (m, 4H), 4.68-4.53 (m, 2H), 4.33 (q, J = 7.2 |
| | | | | Hz, 2H), 4.21 (q, J = 7.2 Hz, 1H), 4.04-3.74 (m, 3H), 3.64 (br |
| | | | | dd, J = 6.4, 14.9 Hz, 2H), 3.45-3.29 (m, 2H), 2.77 (s, 3H), |
| | | | | 2.70-2.65 (m, 1H), 2.59-2.44 (m, 2H), 1.61-1.43 (m, 3H), |
| | | | | 1.36-1.21 (m, 3H), 0.73 (br d, J = 5.2 Hz, 1H), 0.46-0.26 (m, |
| | | | | 2H), 0.10-−0.09 (m, 2H) |
| I-550 ⁱ | QV | QW | 721.5 | 7.78 (d, J = 1.6 Hz, 1H), 7.73-7.67 (m, 2H), 7.39-7.33 (m, |
| | | | | 1H), 7.26-7.20 (m, 1H), 6.94 (s, 1H), 6.79-6.63 (m, 1H), |
| | | | | 6.09-5.98 (m, 1H), 4.37-4.21 (m, 1H), 4.13-3.95 (m, 1H), |
| | | | | 3.83 (s, 3H), 3.76-3.56 (m, 9H), 3.25-3.23 (m, 4H), 2.80 (d, J = |
| | | | | 7.6 Hz, 2H), 2.20 (s, 5H), 2.01-1.92 (m, 1H), 1.37-1.32 |
| | | | | (m, 1H), 1.28 (t, J = 7.6 Hz, 3H), 1.14-1.06 (m, 1H), 0.53- |
| | | | | 0.42 (m, 1H), 0.14-0.04 (m, 2H), −0.21-−0.30 (m, 2H) |

^{a}The coupling was run anywhere from 0-25° C. for 20 min to 2 hr. Final products purified under standard conditions including reverse phase and prep HPLC under a variety of solvent conditions.
^{b} No HOBt employed in the coupling.
^{c} MeOH was used as the solvent.
^{d} The product of the coupling was further deprotected with HCl in dioxane in DCM at rt for 2 hr.
^{e} HOAt used in place of HOBt.
^{f} CDCl₃ was used as the ¹HNMR solvent.
^{g} LCMS reported as (M/2 + H)⁺.
^{h} The product of the coupling was further deprotected by TFA in DCM at rt for 1 hr.
ⁱ The product of the coupling was hydrolyzed with NaOH or LiOH at rt THF, MeOH and water for 1 hr.

TABLE 5
Compounds synthesized via Method 1, coupling of the corresponding amines and carboxylic acids

| | LCMS (ESI+) | |
|---|---|---|
| I-# | m/z (M + H)⁺ | ¹H NMR (400 MHz, DMSO-d₆) δ |
| I-815 | 410.2 | 7.83-7.73 (m, 1H), 7.70-7.63 (m, 2H), 7.62-7.52 (m, 2H), 7.40 (d, J = 5.9 Hz, |
| | | 1H), 6.38-6.27 (m, 1H), 4.41 (br s, 2H), 3.63 (br t, J = 5.0 Hz, 2H), 3.30-3.20 |
| | | (m, 5H), 3.11-2.89 (m, 5H), 2.36-2.31 (m, 1H), 2.26 (br s, 1H) |
| I-816 | 410.1 | 8.16-8.01 (m, 2H), 7.88 (d, J = 8.4 Hz, 1H), 7.78 (s, 1H), 7.69 (d, J = 1.2 Hz, |
| | | 1H), 7.63-7.51 (m, 1H), 6.57-6.31 (m, 1H), 4.64 (t, J = 7.2 Hz, 2H), 4.48- |
| | | 4.34 (m, 2H), 3.60 (td, J = 5.6, 16.8 Hz, 2H), 3.26-2.96 (m, 8H), 2.39-2.25 (m, 2H) |
| I-817 | 422.3 | 8.05 (s, 1H), 7.75 (s, 1H), 7.66 (s, 1H), 7.63-7.57 (m, 1H), 7.56-7.47 (m, 1H), |
| | | 7.33 (s, 1H), 6.28 (s, 1H), 4.66 (t, J = 6.8 Hz, 2H), 4.39 (s, 2H), 3.61 (s, 2H), |
| | | 3.50 (t, J = 7.2 Hz, 2H), 3.13 (s, 3H), 3.12-3.09 (m, 2H), 2.33 (s, 2H), 1.65 (sxt, |
| | | J = 7.2 Hz, 2H), 0.89 (t, J = 7.6 Hz, 3H) |
| I-818 | 408.2 | 8.00 (s, 1H), 7.74 (s, 1H), 7.65 (s, 1H), 7.62-7.57 (m, 1H), 7.54-7.48 (m, 1H), |
| | | 7.35 (s, 1H), 6.25 (s, 1H), 4.62 (dd, J = 7.6, 13.6 Hz, 1H), 4.46-4.30 (m, 3H), |
| | | 3.61 (s, 3H), 3.15 (s, 6H), 2.32-2.20 (m, 2H), 1.10 (d, J = 6.8 Hz, 3H) |
| I-819 | 394.1 | 12.08 (s, 1H), 8.45 (dd, J = 2.0, 18.8 Hz, 1H), 8.15-7.98 (m, 2H), 7.70 (d, J = |
| | | 3.2 Hz, 1H), 6.79 (dd, J = 2.0, 8.4 Hz, 1H), 6.30 (d, J = 14.4 Hz, 1H), 4.64 (t, J = |
| | | 6.8 Hz, 2H), 4.39 (d, J = 8.8 Hz, 2H), 3.60 (td, J = 5.6, 17.6 Hz, 2H), 3.27-3.19 |
| | | (m, 3H), 3.17-3.11 (m, 2H), 3.10-2.97 (m, 3H), 2.36-2.22 (m, 2H) |
| I-820 | 405.1 | 9.06 (t, J = 3.6 Hz, 1H), 7.82-7.72 (m, 1H), 7.69-7.59 (m, 3H), 7.58-7.50 (m, |
| | | 1H), 7.39 (d, J = 7.2 Hz, 1H), 6.31 (d, J = 13.2 Hz, 1H), 4.41 (d, J = 12.0 Hz, |
| | | 2H), 3.69-3.55 (m, 2H), 3.25 (d, J = 1.2 Hz, 3H), 3.18 (t, J = 7.5 Hz, 2H), 3.03 |
| | | (s, 3H), 2.98 (d, J = 7.6 Hz, 2H), 2.37-2.24 (m, 2H) |
| I-821 | 409.3 | 11.54-11.50 (m, 1H), 7.69-7.67 (m, 1H), 7.60-7.55 (m, 2H), 7.50-7.40 (m, |
| | | 1H), 7.25-7.15 (m, 1H), 6.82 (s, 1H), 6.35-6.20 (m, 1H), 4.39 (s, 2H), 3.70- |
| | | 3.55 (m, 2H), 3.40-3.25 (m, 6H), 3.20-3.05 (m, 2H), 3.00-2.85 (m, 2H), 2.40- |
| | | 2.20 (m, 2H) |
| I-822 | 457.3 | 11.26 (s, 1H), 8.35-8.20 (m, 1H), 7.63-7.55 (m, 1H), 7.45 (s, 1H), 7.30-7.10 |
| | | (m, 5H), 6.82 (s, 1 H), 6.35-6.20 (m, 1H), 5.48-5.38 (m, 1H), 4.95-4.70 (m, |
| | | 1H), 4.55-4.35 (m, 2H), 4.35-4.20 (m, 1H), 3.85-3.50 (m, 2H), 3.25-3.10 |
| | | (m, 6H), 3.08-3.00 (m, 2H), 2.45-2.35 (m, 2H) |
| I-823 | 568.3 | 8.37-8.24 (m, 1H), 7.90-7.67 (m, 1H), 7.66-7.38 (m, 2H), 7.28-7.10 (m, |
| | | 4H), 6.40-6.32 (m, 1H), 5.59-5.26 (m, 1H), 5.03-4.59 (m, 1H), 4.57-4.41 |
| | | (m, 2H), 4.39-4.14 (m, 1H), 3.91-3.37 (m, 5H), 3.30-3.23 (m, 3H), 3.15- |
| | | 2.98 (m, 3H), 2.87-2.79 (m, 2H), 2.73-2.64 (m, 2H), 2.45-2.07 (m, 4H) |
| I-824 | 411 | 9.45 (s, 1H), 7.82-7.72 (m, 1H), 7.66-7.62 (m, 1H), 7.61-7.51 (m, 1H), 7.39 |
| | | (d, J = 6.9 Hz, 1H), 6.32 (br d, J = 11.4 Hz, 1H), 4.41 (br s, 2H), 3.67-3.57 (m, |
| | | 2H), 3.26 (br s, 6H), 3.06-2.96 (m, 4H), 2.38-2.22 (m, 2H) |
| I-825 | 419.1 | 11.68-11.46 (m, 1H), 7.63-7.52 (m, 2H), 7.49-7.34 (m, 2H), 7.21-7.11 (m, |
| | | 1H), 6.85 (s, 1H), 6.48-6.35 (m, 1H), 6.34-6.26 (m, 1H), 6.24 (s, 1H), 5.96 |
| | | (q, J = 6.8 Hz, 1H), 4.71-4.06 (m, 2H), 3.78-3.51 (m, 2H), 3.32-2.84 (m, 6H), |
| | | 2.37-2.03 (m, 2H), 1.45 (d, J = 6.8 Hz, 3H) |
| I-826 | 392.2 | 11.58-11.43 (m, 1H), 7.73 (d, J = 2.4 Hz, 1H), 7.58 (d, J = 8.4 Hz, 1H), 7.47- |
| | | 7.35 (m, 2H), 7.26-7.15 (m, 1H), 6.86 (d, J = 2.0 Hz, 1H), 6.26 (d, J = 4.0 Hz, |
| | | 1H), 6.22-6.17 (m, 1H), 4.46-4.26 (m, 4H), 3.65-3.53 (m, 2H), 3.32-3.04 |
| | | (m, 6H), 3.03-2.96 (m, 2H), 2.37-2.17 (m, 2H) |
| I-827 | 407.1 | 11.61-11.27 (m, 1H), 7.82 (s, 1H), 7.62-7.48 (m, 1H), 7.46-7.33 (m, 1H), |
| | | 7.25-7.09 (m, 1H), 6.86 (d, J = 2.0 Hz, 1H), 6.27 (br d, J = 4.0 Hz, 1H), 4.55 (br |
| | | t, J = 6.8 Hz, 2H), 4.43-4.29 (m, 2H), 3.69-3.51 (m, 2H), 3.30-2.96 (m, 8H), |
| | | 2.40-2.24 (m, 2H), 2.23-2.16 (m, 3H) |
| I-828 | 410.1 | 11.52 (br d, J = 6.4 Hz, 1H), 9.48-9.42 (m, 1H), 7.58 (d, J = 8.4 Hz, 1H), 7.36 |
| | | (s, 1H), 7.26-7.15 (m, 1H), 6.92-6.81 (m, 1H), 6.28 (br s, 1H), 4.43-4.34 (m, |
| | | 2H), 3.64-3.55 (m, 2H), 3.41-3.35 (m, 3H), 3.31-3.04 (m, 6H), 3.02-2.96 |
| | | (m, 1H), 2.37-2.33 (m, 1H), 2.28-2.22 (m, 1H), 2.11 (s, 1H), 2.09 (s, 1H) |
| I-829 | 393.4 | 11.62-11.46 (m, 1H), 8.86-8.67 (m, 1H), 7.58 (d, J = 8.4 Hz, 1H), 7.47-7.36 |
| | | (m, 1H), 7.25-7.14 (m, 1H), 6.86 (s, 1H), 6.57-6.45 (m, 1H), 6.28 (br d, J = 4.0 |
| | | Hz, 1H), 4.39 (br s, 2H), 3.66-3.56 (m, 2H), 3.30-2.98 (m, 6H), 2.95-2.89 (m, |
| | | 2H), 2.88-2.79 (m, 2H), 2.35 (br s, 2H) |
| I-830 | 396.1 | 8.13-8.07 (m, 1H), 7.72-7.66 (m, 1H), 7.65-7.58 (m, 2H), 7.42-7.33 (m, |
| | | 2H), 4.60 (q, J = 6.8 Hz, 2H), 4.51-4.44 (m, 1H), 3.92-3.82 (m, 1H), 3.28- |
| | | 3.20 (m, 3H), 3.12-2.99 (m, 6H), 2.81-2.60 (m, 2H), 1.93 (br d, J = 10.6 Hz, |
| | | 1H), 1.75 (br d, J = 10.5 Hz, 2H), 1.54-1.38 (m, 1H) |
| I-831 | 393.1 | 11.57-11.49 (m, 1H), 8.81-8.76 (m, 1H), 7.59 (d, J = 8.4 Hz, 1H), 7.46-7.39 |
| | | (m, 1H), 7.25-7.18 (m, 1H), 6.87 (s, 1H), 6.60-6.49 (m, 1H), 6.32-6.27 (m, |
| | | 1H), 4.40 (s, 2H), 3.66-3.60 (m, 2H), 3.24-2.99 (m, 6H), 2.95-2.90 (m, 2H), |
| | | 2.88-2.81 (m, 2H), 2.39-2.33 (m, 2H) |
| I-832 | 500.3 | 7.99-7.94 (m, 1H), 7.71 (d, J = 1.3 Hz, 1H), 7.63 (s, 1H), 7.62-7.55 (m, 1H), |
| | | 7.52-7.46 (m, 1H), 7.40-7.36 (m, 1H), 7.34-7.31 (m, 1H), 7.28 (d, J = 7.5 Hz, |
| | | 1H), 7.25-7.19 (m, 1H), 7.16 (t, J = 8.0 Hz, 1H), 6.20-6.09 (m, 1H), 5.25- |
| | | 5.18 (m, 1H), 5.03-4.93 (m, 1H), 4.93-4.79 (m, 1H), 4.67-4.54 (m, 1H), 4.49- |
| | | 4.32 (m, 4H), 4.12 (d, J = 17.2 Hz, 1H), 3.74-3.47 (m, 2H), 3.25 (s, 3H), 3.03 |
| | | (s, 3H), 2.24-2.06 (m, 1H), 1.78-1.63 (m, 1H) |
| I-833 | 500.2 | 7.99-7.94 (m, 1H), 7.71 (d, J = 1.5 Hz, 1H), 7.64-7.55 (m, 2H), 7.52-7.47 (m, |
| | | 1H), 7.40-7.36 (m, 1H), 7.34-7.31 (m, 1H), 7.28 (d, J = 7.2 Hz, 1H), 7.25- |
| | | 7.19 (m, 1H), 7.19-7.13 (m, 1H), 6.20-6.09 (m, 1H), 5.27-5.17 (m, 1H), 5.05- |
| | | 4.79 (m, 2H), 4.67-4.56 (m, 1H), 4.49-4.34 (m, 4H), 4.12 (d, J = 16.8 Hz, |
| | | 1H), 3.76-3.69 (m, 1H), 3.63-3.47 (m, 2H), 3.25 (s, 3H), 3.13-2.92 (m, 3H), |
| | | 2.22-2.05 (m, 1H), 1.76-1.65 (m, 1H) |
| I-834 | 419.1 | 11.54-11.45 (m, 1H), 7.55-7.48 (m, 2H), 7.43-7.31 (m, 2H), 7.14-7.08 (m, |
| | | 1H), 6.84-6.76 (m, 1H), 6.42-6.30 (m, 1H), 6.29-6.21 (m, 1H), 6.21-6.13 |
| | | (m, 1H), 5.90 (q, J = 6.8 Hz, 1H), 4.21 (d, J = 17.6 Hz, 2H), 3.68-3.51 (m, 2H), |
| | | 3.23 (s, 6H), 2.21 (d, J = 12.4 Hz, 1H), 2.14-1.99 (m, 1H), 1.42-1.36 (m, 3H) |
| I-835 | 495.2 | 7.96 (s, 1H), 7.81-7.75 (m, 1H), 7.74-7.70 (m, 1H), 7.68 (d, J = 8.0 Hz, 1H), |
| | | 7.65-7.60 (m, 2H), 7.60-7.54 (m, 1H), 7.52 (d, J = 10.0 Hz, 1H), 7.50-7.43 |
| | | (m, 1H), 7.41-7.33 (m, 1H), 6.22-6.07 (m, 1H), 5.07-4.92 (m, 2H), 4.80- |
| | | 4.67 (m, 1H), 4.49-4.34 (m, 2H), 4.25-4.16 (m, 1H), 3.82-3.73 (m, 1H), 3.69- |
| | | 3.59 (m, 1H), 3.58-3.43 (m, 1H), 3.25 (s, 3H), 3.02 (s, 3H), 2.19 (s, 1H), 1.81- |
| | | 1.65 (m, 1H) |
| I-837 | 464.2 | 12.05 (br s, 1H), 7.42-7.38 (m, 1H), 7.35 (br d, J = 7.2 Hz, 1H), 7.15 (d, J = 8.4 |
| | | Hz, 1H), 7.05 (t, J = 7.2 Hz, 1H), 6.95-6.89 (m, 1H), 6.46 (br s, 1H), 6.12 (br s, |
| | | 1H), 4.40-4.34 (m, 2H), 3.73 (s, 3H), 3.69-3.65 (m, 2H), 3.21-3.00 (m, 6H), |
| | | 2.99-2.93 (m, 1H), 2.37 (br s, 1H), 2.26 (br s, 1H), 1.06-1.00 (m, 6H) |
| I-838 | 564 | 8.33-8.22 (m, 1H), 7.87-7.66 (m, 1H), 7.57-7.36 (m, 4H), 7.25-7.14 (m, |
| | | 5H), 7.12-7.03 (m, 1H), 6.38-6.30 (m, 1H), 5.55-5.34 (m, 1H), 4.94-4.69 |
| | | (m, 1H), 4.54 (br d, J = 13.6 Hz, 1H), 4.47-4.17 (m, 2H), 3.78-3.68 (m, 4H), |
| | | 3.44 (br s, 1H), 3.24-3.15 (m, 3H), 3.13 (br s, 5H), 2.26 (br s, 2H) |
| I-839 | 564 | 8.33-8.22 (m, 1H), 7.87-7.66 (m, 1H), 7.57-7.36 (m, 4H), 7.25-7.14 (m, |
| | | 5H), 7.12-7.03 (m, 1H), 6.38-6.30 (m, 1H), 5.55-5.34 (m, 1H), 4.94-4.69 |
| | | (m, 1H), 4.54 (br d, J = 13.6 Hz, 1H), 4.47-4.17 (m, 2H), 3.78-3.68 (m, 4H), |
| | | 3.44 (br s, 1H), 3.24-3.15 (m, 3H), 3.13 (br s, 5H), 2.26 (br s, 2H) |
| I-840 | 494.2 | 11.81 (br s, 1H), 8.50-8.45 (m, 1H), 7.74-7.65 (m, 1H), 7.41 (br d, J = 16.4 |
| | | Hz, 1H), 6.90-6.87 (m, 1H), 6.81-6.77 (m, 1H), 6.23-6.16 (m, 1H), 4.50- |
| | | 4.40 (m, 1H), 4.38-4.32 (m, 2H), 3.90-3.79 (m, 1H), 3.19 (br d, J = 12.4 Hz, |
| | | 2H), 3.11 (br d, J = 4.8 Hz, 1H), 3.06 (br d, J = 11.2 Hz, 2H), 2.94-2.85 (m, |
| | | 4H), 2.77-2.65 (m, 1H), 1.87-1.80 (m, 6H), 1.77-1.72 (m, 2H), 1.59-1.40 |
| | | (m, 6H), 1.40-1.23 (m, 2H) |
| I-841 | 572.7 | 12.13-12.07 (m, 1H), 8.15-8.10 (m, 1H), 8.09 (s, 1H), 7.72-7.65 (m, 1H), |
| | | 7.39 (br t, J = 7.6 Hz, 1H), 7.33 (br d, J = 7.2 Hz, 1H), 7.15 (br d, J = 8.4 Hz, |
| | | 1H), 7.04 (t, J = 7.2 Hz, 1H), 6.97-6.87 (m, 1H), 6.38 (br s, 1H), 6.16-6.09 (m, |
| | | 1H), 4.62 (q, J = 7.2 Hz, 2H), 4.38-4.30 (m, 2H), 3.71 (d, J = 3.2 Hz, 3H), 3.66 |
| | | (br d, J = 10.0 Hz, 4H), 3.59 (br t, J = 5.2 Hz, 2H), 3.09 (q, J = 7.2 Hz, 2H), 2.43- |
| | | 2.32 (m, 5H), 2.24 (br s, 4H) |
| I-842 | 543.3 | 12.25-11.67 (m, 1H), 8.14-8.07 (m, 1H), 7.71-7.65 (m, 1H), 7.43-7.33 (m, |
| | | 2H), 7.15 (br d, J = 8.0 Hz, 1H), 7.05 (t, J = 7.6 Hz, 1H), 6.94 (d, J = 6.0 Hz, |
| | | 1H), 6.89 (d, J = 6.0 Hz, 1H), 6.56 (br s, 1H), 6.16-6.09 (m, 1H), 4.65-4.58 |
| | | (m, 2H), 4.37-4.28 (m, 2H), 3.72 (d, J = 3.6 Hz, 3H), 3.71-3.64 (m, 3H), 3.59 |
| | | (br t, J = 5.6 Hz, 1H), 3.52 (br t, J = 6.0 Hz, 2H), 3.12-3.07 (m, 2H), 2.67 (br s, |
| | | 1H), 2.33 (br d, J = 1.6 Hz, 2H), 2.27 (br s, 1H), 1.90 (br d, J = 6.4 Hz, 1H), 1.84 |
| | | (br d, J = 6.0 Hz, 1H) |
| I-843 | 618.4 | 12.16-12.03 (m, 1H), 8.14-8.08 (m, 1H), 7.73-7.64 (m, 1H), 7.42-7.37 (m, |
| | | 1H), 7.33 (dd, J = 1.6, 7.6 Hz, 1H), 7.15 (br d, J = 8.4 Hz, 1H), 7.07-7.01 (m, |
| | | 1H), 6.97-6.87 (m, 1H), 6.37 (br s, 1H), 6.16-6.09 (m, 1H), 4.65-4.59 (m, |
| | | 2H), 4.54 (t, J = 5.6 Hz, 1H), 4.42 (t, J = 6.0 Hz, 1H), 4.38-4.28 (m, 2H), 3.73- |
| | | 3.70 (m, 3H), 3.67-3.57 (m, 5H), 3.30 (s, 3H), 3.09 (q, J = 7.2 Hz, 2H), 2.42- |
| | | 2.34 (m, 5H), 2.27 (br s, 1H), 1.88-1.76 (m, 2H) |
| I-844 | 451.2 | |
| I-845 | 539.2 | |
| I-846 | 630.9 | 12.12 (br d, J = 9.2 Hz, 1H), 8.17-8.04 (m, 1H), 7.71-7.64 (m, 1H), 7.44-7.31 |
| | | (m, 2H), 7.15 (br d, J = 8.4 Hz, 1H), 7.04 (t, J = 7.2 Hz, 1H), 6.98-6.87 (m, |
| | | 1H), 6.40 (br s, 1H), 6.18-6.08 (m, 1H), 4.66-4.58 (m, 2H), 4.38-4.30 (m, |
| | | 2H), 3.76-3.52 (m, 10H), 3.15-3.02 (m, 3H), 2.70-2.62 (m, 2H), 2.42-2.22 |
| | | (m, 5H), 1.61-1.12 (m, 3H), 0.87 (t, J = 7.2 Hz, 3H) |
| I-847 | 626.6 | 12.09-11.96 (m, 1H), 8.13-8.09 (m, 1H), 7.73-7.63 (m, 1H), 7.43-7.31 (m, |
| | | 2H), 7.15 (br d, J = 8.8 Hz, 1H), 7.05 (t, J = 7.2 Hz, 1H), 6.97-6.87 (m, 1H), |
| | | 6.57 (br s, 1H), 6.17-6.08 (m, 1H), 4.65-4.57 (m, 2H), 4.42-4.29 (m, 2H), |
| | | 3.99-3.80 (m, 2H), 3.75-3.57 (m, 7H), 3.09 (q, J = 6.8 Hz, 3H), 2.65 (br d, J = |
| | | 16.0 Hz, 2H), 2.36-2.08 (m, 4H), 1.88-1.73 (m, 1H), 1.66-1.26 (m, 7H) |
| I-848 | 592.4 | 13.05 (br s, 1H), 8.15-8.09 (m, 1H), 8.04 (br s, 1H), 7.78 (br d, J = 7.2 Hz, 1H), |
| | | 7.73-7.65 (m, 1H), 7.42-7.37 (m, 1H), 7.34 (dd, J = 1.6, 7.6 Hz, 1H), 7.16- |
| | | 6.93 (m, 4H), 6.81 (t, J = 2.4 Hz, 1H), 6.16 (br s, 1H), 4.66-4.60 (m, 2H), 4.40 |
| | | (br s, 2H), 4.24 (s, 2H), 3.73-3.70 (m, 3H), 3.69-3.65 (m, 1H), 3.63-3.60 (m, |
| | | 1H), 3.18-3.10 (m, 4H), 2.39-2.28 (m, 2H) |
| I-849 | 650.6 | 12.14 (s, 1H), 8.27 (d, J = 4.8 Hz, 2H), 8.14-8.07 (m, 1H), 7.72-7.63 (m, 1H), |
| | | 7.42-7.31 (m, 2H), 7.21-7.10 (m, 2H), 7.04 (t, J = 7.2 Hz, 1H), 6.98-6.87 (m, |
| | | 1H), 6.56 (t, J = 4.8 Hz, 1H), 6.36 (br s, 1H), 6.17-6.07 (m, 1H), 4.62 (br d, J = |
| | | 8.8 Hz, 2H), 4.40-4.19 (m, 4H), 4.06-3.97 (m, 1H), 3.72 (d, J = 3.2 Hz, 3H), |
| | | 3.65 (s, 1H), 3.59 (br t, J = 5.6 Hz, 1H), 3.09 (br d, J = 7.2 Hz, 3H), 2.38-2.18 |
| | | (m, 3H), 1.91 (br s, 2H), 1.50-1.38 (m, 2H) |
| I-850 | 667.6 | 12.09-11.89 (m, 1H), 8.18-8.07 (m, 1H), 7.79 (br s, 1H), 7.74-7.63 (m, 1H), |
| | | 7.41 (br t, J = 7.6 Hz, 1H), 7.33 (br d, J = 7.2 Hz, 1H), 7.16 (br d, J = 7.8 Hz, |
| | | 1H), 7.06 (br t, J = 7.6 Hz, 1H), 6.96-6.86 (m, 1H), 6.78-6.62 (m, 1H), 6.31 (br |
| | | s, 1H), 6.17-6.05 (m, 1H), 4.67-4.56 (m, 2H), 4.38-4.28 (m, 2H), 3.81 (br s, |
| | | 3H), 3.73 (br s, 4H), 3.67-3.63 (m, 2H), 3.61-3.61 (m, 1H), 3.60 (br d, J = 5.2 |
| | | Hz, 1H), 3.13-3.06 (m, 2H), 2.56 (br s, 2H), 2.36-2.24 (m, 3H), 1.93-1.72 (m, 2H) |
| I-851 | 595.5 | 12.57-12.51 (m, 1H), 12.17-12.11 (m, 1H), 8.13-8.08 (m, 1H), 7.71-7.65 |
| | | (m, 1H), 7.43-7.34 (m, 2H), 7.16 (br d, J = 8.4 Hz, 1H), 7.05 (t, J = 7.2 Hz, |
| | | 1H), 6.99-6.89 (m, 1H), 6.47 (br s, 1H), 6.18-6.08 (m, 1H), 4.80-4.57 (m, |
| | | 4H), 4.41-4.29 (m, 2H), 3.90 (br s, 2H), 3.76-3.72 (m, 3H), 3.68-3.56 (m, |
| | | 2H), 3.09 (q, J = 6.8 Hz, 2H), 2.75 (br t, J = 5.2 Hz, 2H), 2.37-2.25 (m, 2H) |
| I-852 | 726.1 | 12.18 (br d, J = 7.8 Hz, 1H), 8.17-8.07 (m, 1H), 7.71 (br s, 3H), 7.43 (br s, 3H), |
| | | 7.39-7.30 (m, 1H), 7.17 (br d, J = 8.3 Hz, 1H), 7.11-7.02 (m, 1H), 7.01-6.89 |
| | | (m, 1H), 6.51 (br s, 1H), 6.13 (br s, 1H), 4.63 (br d, J = 6.4 Hz, 3H), 4.47-4.19 |
| | | (m, 5H), 3.90-3.53 (m, 7H), 3.19-2.72 (m, 6H), 2.42-2.22 (m, 2H) |
| I-853 | 500.1 | 9.55-9.46 (m, 1H), 8.12 (d, J = 2.9 Hz, 1H), 7.75-7.67 (m, 2H), 7.54-7.38 (m, |
| | | 2H), 7.31-7.24 (m, 1H), 6.83-6.72 (m, 2H), 6.35-6.26 (m, 1H), 4.68-4.60 |
| | | (m, 2H), 4.45-4.37 (m, 2H), 3.61 (td, J = 5.8, 18.0 Hz, 2H), 3.26-2.91 (m, 8H), |
| | | 2.37-2.23 (m, 5H) |
| I-854 | 525.2 | |
| I-855 | 542.2 | |
| I-856 | 512.2 | 8.12 (d, J = 3.5 Hz, 1H), 7.82-7.68 (m, 2H), 7.47-7.33 (m, 2H), 7.16-7.10 (m, |
| | | 2H), 6.42-6.28 (m, 1H), 4.68-4.60 (m, 2H), 4.42 (br d, J = 11.7 Hz, 2H), 3.66- |
| | | 3.55 (m, 2H), 3.23-2.93 (m, 8H), 2.38-2.21 (m, 8H), 2.13-2.05 (m, 3H) |
| I-857 | 587.2 | |
| I-858 | 649.3 | |
| I-859 | 674.2 | |
| I-860 | 648.3 | |
| I-861 | 573.2 | |
| I-862 | 635.2 | 12.26-12.02 (m, 1H), 8.16-8.09 (m, 2H), 7.73-7.66 (m, 1H), 7.60-7.52 (m, |
| | | 1H), 7.44-7.33 (m, 2H), 7.16 (br d, J = 8.5 Hz, 1H), 7.06 (t, J = 7.4 Hz, 1H), |
| | | 6.85 (d, J = 8.5 Hz, 2H), 6.68 (dd, J = 5.1, 6.9 Hz, 1H), 6.46 (br s, 1H), 6.19- |
| | | 6.10 (m, 1H), 4.63 (br d, J = 7.7 Hz, 2H), 4.42-4.30 (m, 2H), 3.84-3.72 (m, |
| | | 7H), 3.59 (br d, J = 3.6 Hz, 6H), 3.10 (br d, J = 6.7 Hz, 2H), 2.36 (br d, J = 1.4 |
| | | Hz, 2H) |
| I-863 | 636.2 | |
| I-864 | 600.2 | |
| I-865 | 603.2 | |
| I-866 | 629.2 | 12.22-12.00 (m, 1H), 8.15-8.08 (m, 1H), 7.72-7.66 (m, 1H), 7.44-7.32 (m, |
| | | 2H), 7.16 (br d, J = 8.1 Hz, 1H), 7.05 (t, J = 7.5 Hz, 1H), 6.99-6.88 (m, 1H), |
| | | 6.42 (br s, 1H), 6.18-6.09 (m, 1H), 4.67-4.58 (m, 2H), 4.41-4.29 (m, 2H), |
| | | 3.76-3.57 (m, 9H), 3.21-3.06 (m, 6H), 2.76 (s, 6H), 2.40-2.24 (m, 2H) |
| I-867 | 650.2 | 12.22-12.06 (m, 1H), 8.89 (s, 1H), 8.17-8.07 (m, 1H), 7.75-7.64 (m, 1H), |
| | | 7.45-7.33 (m, 2H), 7.16 (br d, J = 8.0 Hz, 1H), 7.06 (t, J = 7.3 Hz, 1H), 7.00- |
| | | 6.88 (m, 1H), 6.81 (d, J = 8.8 Hz, 2H), 6.66 (d, J = 8.9 Hz, 2H), 6.43 (br d, J = |
| | | 2.0 Hz, 1H), 6.19-6.09 (m, 1H), 4.68-4.59 (m, 2H), 4.42-4.30 (m, 2H), 3.85- |
| | | 3.57 (m, 9H), 3.15-2.96 (m, 6H), 2.35 (br d, J = 2.3 Hz, 2H |
| I-868 | 682.2 | |
| I-869 | 525.2 | |
| I-870 | 603.2 | |
| I-871 | 550.2 | 8.93 (br d, J = 1.5 Hz, 1H), 8.96-8.91 (m, 1H), 8.82-8.75 (m, 1H), 8.10 (s, |
| | | 1H), 7.92 (s, 1H), 7.73-7.64 (m, 3H), 7.62-7.57 (m, 1H), 7.30 (d, J = 13.0 Hz, |
| | | 1H), 6.98-6.89 (m, 1H), 6.71-6.63 (m, 2H), 6.12-6.00 (m, 1H), 5.00 (br d, J = |
| | | 5.6 Hz, 2H), 4.61 (br d, J = 1.4 Hz, 1H), 4.30-4.19 (m, 2H), 3.59-3.48 (m, 2H), |
| | | 3.25 (br d, J = 1.5 Hz, 2H), 3.14-2.95 (m, 6H), 2.23 (br d, J = 2.4 Hz, 2H) |
| I-872 | 517.2 | |
| I-873 | 513.2 | 8.15-8.09 (m, 1H), 7.75-7.67 (m, 1H), 7.34-7.23 (m, 1H), 7.01-6.88 (m, |
| | | 1H), 6.73 (s, 1H), 6.29-6.18 (m, 1H), 6.08-5.98 (m, 1H), 4.67-4.59 (m, 2H), |
| | | 4.39-4.28 (m, 2H), 3.64-3.54 (m, 2H), 3.42-3.36 (m, 2H), 3.18 (br s, 6H), |
| | | 2.74-2.60 (m, 3H), 2.46-2.21 (m, 5H), 1.36-1.15 (m, 1H) |
| I-874 | 477.2 | |
| I-875 | 660.1 | |
| I-876 | 633.2 | |
| I-877 | 567.1 | |
| I-878 | 539.2 | |
| I-879 | 437.1 | |
| I-880 | 438.1 | |
| I-881 | 454.1 | |
| I-882 | 460.2 | 12.24 (br s, 1H), 8.09 (s, 1H), 7.91-7.83 (m, 2H), 7.49-7.43 (m, 1H), 7.14 (d, |
| | | J = 6.1 Hz, 1H), 6.97-6.82 (m, 1H), 6.20 (br s, 1H), 4.41-4.27 (m, 2H), 4.11 (s, |
| | | 3H), 3.68-3.58 (m, 2H), 3.25-3.01 (m, 6H), 2.40 (br d, J = 3.9 Hz, 2H), 2.12- |
| | | 2.06 (m, 3H) |
| I-883 | 460.1 | 12.70 (s, 1H), 12.34-12.11 (m, 1H), 8.04-7.99 (m, 1H), 7.93 (s, 1H), 7.71- |
| | | 7.63 (m, 1H), 7.58-7.51 (m, 1H), 7.13-7.03 (m, 1H), 6.88 (br s, 1H), 6.19 (br |
| | | d, J = 1.6 Hz, 1H), 4.43-4.33 (m, 2H), 3.69-3.59 (m, 2H), 3.30-2.92 (m, 6H), |
| | | 2.56-2.54 (m, 3H), 2.39 (br d, J = 1.7 Hz, 2H), 2.09 (d, J = 7.0 Hz, 3H) |
| I-884 | 445.1 | |
| I-885 | 421.1 | |
| I-886 | 436.1 | |
| I-887 | 458.1 | |
| I-888 | 490.1 | |
| I-889 | 461 | 13.15 (br s, 1H), 12.34-12.02 (m, 1H), 8.15 (s, 1H), 8.03 (s, 1H), 7.66 (s, 2H), |
| | | 7.13-7.03 (m, 1H), 6.89 (d, J = 2.7 Hz, 1H), 6.24-6.16 (m, 1H), 4.41-4.35 (m, |
| | | 2H), 3.68-3.59 (m, 2H), 3.24-3.02 (m, 6H), 2.43-2.25 (m, 2H), 2.09 (d, J = |
| | | 6.7 Hz, 3H) |
| I-890 | 420.1 | |
| I-891 | 440.1 | |
| I-892 | 461.1 | |
| I-893 | 568.1 | |
| I-894 | 517.2 | |
| I-895 | 524.1 | |
| I-896 | 520.1 | |
| I-897 | 460.2 | |
| I-898 | 612.4 | 12.05-11.97 (m, 1H), 8.13-8.08 (m, 1H), 7.71-7.65 (m, 1H), 7.42-7.34 (m, |
| | | 2H), 7.15 (br d, J = 8.0 Hz, 1H), 7.04 (t, J = 7.2 Hz, 1H), 6.98-6.88 (m, 1H), |
| | | 6.57 (br s, 1H), 6.15-6.10 (m, 1H), 4.65-4.59 (m, 2H), 4.38-4.31 (m, 2H), |
| | | 3.98-3.76 (m, 2H), 3.73 (br s, 3H), 3.65 (br t, J = 5.6 Hz, 1H), 3.59 (br t, J = 5.6 |
| | | Hz, 2H), 3.50-3.41 (m, 1H), 3.13-3.07 (m, 3H), 2.34 (br d, J = 2.4 Hz, 1H), |
| | | 2.26 (br d, J = 14.0 Hz, 4H), 2.16-2.07 (m, 1H), 1.91-1.78 (m, 1H), 1.72-1.65 |
| | | (m, 1H), 0.50-0.37 (m, 2H), 0.36-0.29 (m, 2H) |
| I-899 | 553.2 | 8.11 (d, J = 2.4 Hz, 1H), 7.69 (s, 1H), 7.62-7.55 (m, 1H), 7.49-7.42 (m, 1H), |
| | | 7.34-7.28 (m, 1H), 7.16-7.04 (m, 2H), 6.90 (d, J = 1.6 Hz, 1H), 6.75-6.54 (m, |
| | | 1H), 6.03-5.93 (m, 1H), 4.62 (t, J = 6.8 Hz, 2H), 4.32-4.10 (m, 2H), 3.70 (s, |
| | | 3H), 3.67-3.46 (m, 4H), 3.32 (s, 3H), 3.13-3.06 (m, 2H), 2.34-2.21 (m, 2H), |
| | | 0.11-−0.01 (m, 2H), −0.38 (s, 2H) |
| I-900 | 665.5 | 2.3-2.4 (m, 2 H), 2.6-2.6 (m, 2 H), 3.1-3.1 (m, 2 H), 3.5-3.6 (m, 5 H), 3.6 |
| | | (br s, 1 H), 3.7-3.7 (m, 2 H), 3.8 (s, 6 H), 4.3-4.4 (m, 2 H), 4.6-4.6 (m, 2 H), |
| | | 6.1-6.2 (m, 1 H), 6.3-6.3 (m, 2 H), 6.4-6.5 (m, 1 H), 6.9-7.0 (m, 1 H), 7.0- |
| | | 7.1 (m, 1 H), 7.1-7.2 (m, 1 H), 7.3-7.4 (m, 1 H), 7.4-7.4 (m, 1 H), 7.6-7.7 |
| | | (m, 1 H), 7.9-8.0 (m, 1 H), 8.1-8.1 (m, 1 H), 12.1-12.1 (m, 1 H) |
| I-901 | 635.3 | 12.22-12.12 (m, 1H), 8.16-8.09 (m, 1H), 7.73-7.65 (m, 1H), 7.45-7.34 (m, |
| | | 2H), 7.21-7.15 (m, 2H), 7.12-7.03 (m, 1H), 7.00-6.90 (m, 1H), 6.85 (br d, J = |
| | | 6.8 Hz, 1H), 6.79 (d, J = 7.6 Hz, 1H), 6.50 (br s, 1H), 6.19-6.10 (m, 1H), 4.81- |
| | | 4.59 (m, 4H), 4.42-4.28 (m, 2H), 3.93-3.82 (m, 3H), 3.76 (d, J = 3.2 Hz, 5H), |
| | | 3.66 (br t, J = 5.6 Hz, 1H), 3.60 (br t, J = 5.6 Hz, 1H), 3.10 (q, J = 6.8 Hz, 2H), |
| | | 2.87 (br t, J = 5.2 Hz, 2H), 2.39-2.25 (m, 2H) |
| I-902 | 478.2 | 12.26-12.12 (m, 1H), 7.95-7.86 (m, 1H), 7.57-7.47 (m, 1H), 7.44-7.35 (m, |
| | | 1H), 7.20-7.02 (m, 2H), 6.99-6.91 (m, 1H), 6.26-6.16 (m, 1H), 4.42 (d, J = |
| | | 6.8 Hz, 2H), 3.94 (s, 7H), 3.77-3.67 (m, 2H), 3.49 (s, 4H), 2.52-2.35 (m, 2H), |
| | | 2.24-2.14 (m, 3H) |
| I-903 | 745.3 | |
| I-904 | 712.3 | 12.17 (br d, J = 7.1 Hz, 1H), 8.48 (d, J = 5.0 Hz, 1H), 8.20-8.07 (m, 3H), 7.73- |
| | | 7.67 (m, 1H), 7.56-7.48 (m, 3H), 7.44-7.34 (m, 2H), 7.27 (br d, J = 5.1 Hz, |
| | | 1H), 7.17 (br d, J = 8.1 Hz, 1H), 7.10-7.04 (m, 1H), 7.01-6.90 (m, 1H), 6.49 |
| | | (br s, 1H), 6.15 (br d, J = 10.5 Hz, 1H), 4.71-4.57 (m, 2H), 4.43-4.29 (m, 2H), |
| | | 3.93 (br s, 4H), 3.82 (br s, 4H), 3.76 (br s, 3H), 3.69-3.58 (m, 2H), 3.16-3.05 |
| | | (m, 2H), 2.36 (br s, 2H) |
| I-905 | 715.3 | |
| I-906 | 765.3 | |
| I-907 | 735.3 | |
| I-908 | 652.3 | |
| I-909 | 689.3 | |
| I-910 | 722.3 | |
| I-911 | 648.5 | 12.16-12.13 (m, 1H), 8.13-8.08 (m, 1H), 7.70-7.65 (m, 1H), 7.42-7.36 (m, |
| | | 2H), 7.32 (br d, J = 7.2 Hz, 5H), 7.12-7.01 (m, 2H), 6.95-6.85 (m, 1H), 6.41- |
| | | 6.36 (m, 1H), 6.15-6.08 (m, 1H), 4.64-4.58 (m, 2H), 4.40-4.13 (m, 4H), 3.64 |
| | | (br d, J = 6.0 Hz, 1H), 3.59 (br s, 1H), 3.42 (br s, 3H), 3.12-3.07 (m, 2H), 3.06 |
| | | (br s, 1H), 2.95 (br d, J = 10.4 Hz, 2H), 2.33 (br s, 2H), 2.29-2.12 (m, 2H), 1.95 |
| | | (s, 3H) |
| I-912 | 691.3 | 12.02 (br d, J = 9.2 Hz, 1H), 8.14-8.06 (m, 1H), 7.72-7.61 (m, 2H), 7.42-7.32 |
| | | (m, 2H), 7.17-7.08 (m, 2H), 7.04 (t, J = 7.2 Hz, 1H), 6.98-6.87 (m, 1H), 6.63 |
| | | (dd, J = 4.8, 7.6 Hz, 1H), 6.58 (br s, 1H), 6.16-6.08 (m, 1H), 4.67-4.55 (m, |
| | | 2H), 4.40-4.28 (m, 2H), 3.99 (br s, 1H), 3.90-3.81 (m, 1H), 3.77-3.68 (m, |
| | | 8H), 3.66-3.57 (m, 3H), 3.48 (br dd, J = 11.2, 20.0 Hz, 4H), 3.09 (q, J = 7.2 Hz, |
| | | 2H), 3.03-2.89 (m, 2H), 2.40-2.22 (m, 2H) |
| I-913 | 678.3 | 12.14 (br d, J = 7.6 Hz, 1H), 8.17-8.05 (m, 1H), 7.73-7.64 (m, 1H), 7.43-7.36 |
| | | (m, 1H), 7.35 (br d, J = 7.6 Hz, 1H), 7.15 (br d, J = 8.0 Hz, 1H), 7.03 (td, J = 7.6, |
| | | 12.8 Hz, 2H), 6.97-6.88 (m, 1H), 6.80 (t, J = 7.2 Hz, 2H), 6.40 (br s, 1H), 6.19- |
| | | 6.09 (m, 1H), 4.66-4.59 (m, 2H), 4.46-4.12 (m, 4H), 3.74-3.70 (m, 6H), 3.67- |
| | | 3.58 (m, 2H), 3.24 (br s, 4H), 3.12-3.07 (m, 2H), 2.77 (br s, 2H), 2.35 (br d, J = |
| | | 1.6 Hz, 1H), 2.29 (s, 4H) |
| I-914 | 690.3 | |
| I-915 | 688 | |
| I-916 | 735.3 | |
| I-917 | 704.3 | |
| I-918 | 687.3 | |
| I-919 | 689.3 | |
| I-920 | 690.3 | |
| I-921 | 688.3 | |
| I-922 | 724.3 | |
| I-923 | 700.3 | |
| I-924 | 703.3 | |
| I-925 | 702 | |
| I-926 | 704 | 12.17 (br s, 1H), 8.18-8.02 (m, 2H), 7.83 (d, J = 2.3 Hz, 1H), 7.75-7.63 (m, |
| | | 1H), 7.47-7.32 (m, 2H), 7.16 (br d, J = 8.3 Hz, 1H), 7.06 (t, J = 7.4 Hz, 1H), |
| | | 7.00-6.89 (m, 1H), 6.61 (d, J = 2.1 Hz, 1H), 6.47 (d, J = 2.6 Hz, 1H), 6.35 (d, |
| | | J = 1.5 Hz, 1H), 6.19-6.08 (m, 1H), 4.68-4.58 (m, 2H), 4.43-4.27 (m, 2H), 3.90 |
| | | (br s, 4H), 3.78 (s, 3H), 3.76-3.72 (m, 3H), 3.68-3.58 (m, 2H), 3.20 (br s, 4H), |
| | | 3.10 (q, J = 6.9 Hz, 2H), 2.35 (br d, J = 2.2 Hz, 2H) |
| I-927 | 701.3 | |
| I-928 | 702.3 | |
| I-929 | 704.3 | |
| I-930 | 700.3 | |
| I-931 | 706.3 | 12.16 (br s, 1H), 8.17-8.06 (m, 1H), 7.80 (d, J = 8.9 Hz, 2H), 7.74-7.65 (m, |
| | | 1H), 7.46-7.31 (m, 2H), 7.17-6.91 (m, 5H), 6.48 (br d, J = 2.3 Hz, 1H), 6.22- |
| | | 6.07 (m, 1H), 4.70-4.56 (m, 2H), 4.40-4.30 (m, 2H), 4.24 (q, J = 7.1 Hz, 2H), |
| | | 3.83 (br s, 4H), 3.77-3.71 (m, 3H), 3.68-3.57 (m, 2H), 3.43 (br s, 4H), 3.10 (q, |
| | | J = 6.9 Hz, 2H), 2.40-2.23 (m, 2H), 1.29 (t, J = 7.1 Hz, 3H) |
| I-932 | 720.3 | |
| I-933 | 638.3 | |
| I-934 | 653.5 | |
| I-935 | 703.4 | |
| I-936 | 674.3 | |
| I-937 | 703.2 | |
| I-938 | 661.3 | 12.37-12.18 (m, 1H), 8.18-8.01 (m, 1H), 7.90-7.76 (m, 1H), 7.72-7.60 (m, |
| | | 3H), 7.57-7.45 (m, 3H), 7.44-7.37 (m, 1H), 7.12-7.01 (m, 1H), 6.98-6.87 |
| | | (m, 1H), 6.82 (br s, 1H), 6.17 (br s, 1H), 4.68-4.58 (m, 2H), 4.42-4.31 (m, |
| | | 2H), 3.88 (td, J = 3.0, 6.0 Hz, 1H), 3.82-3.70 (m, 4H), 3.67-3.64 (m, 1H), 3.63- |
| | | 3.58 (m, 2H), 3.19-3.05 (m, 3H), 2.43-2.19 (m, 3H), 0.85-0.65 (m, 4H) |
| I-939 | 679.3 | 12.11 (d, J = 6.8 Hz, 1H), 8.12-8.08 (m, 1H), 7.71-7.66 (m, 1H), 7.62 (s, 1H), |
| | | 7.41-7.34 (m, 2H), 7.17-7.12 (m, 2H), 7.05 (t, J = 7.2 Hz, 1H), 6.97-6.89 (m, |
| | | 1H), 6.42 (s, 1H), 6.15-6.10 (m, 1H), 4.65-4.60 (m, 2H), 4.38-4.31 (m, 2H), |
| | | 3.79 (s, 7H), 3.72 (d, J = 3.2 Hz, 3H), 3.65 (t, J = 5.2 Hz, 1H), 3.60 (t, J = 5.2 |
| | | Hz, 1H), 3.25 (s, 4H), 3.12-3.07 (m, 2H), 2.36-2.26 (m, 2H), 2.20 (s, 3H) |
| I-940 | 690.3 | 12.17-12.08 (m, 1H), 8.25-8.17 (m, 1H), 8.15-8.08 (m, 1H), 7.72 (s, 1H), |
| | | 7.60 (d, J = 1.6 Hz, 1H), 7.43-7.34 (m, 2H), 7.16 (br d, J = 8.8 Hz, 1H), 7.09- |
| | | 7.02 (m, 1H), 6.98-6.88 (m, 1H), 6.46 (br s, 1H), 6.18-6.09 (m, 1H), 4.60 (br s, |
| | | 2H), 4.42-4.31 (m, 2H), 3.85 (s, 3H), 3.79 (br s, 4H), 3.73 (br d, J = 3.2 Hz, |
| | | 3H), 3.66 (br s, 4H), 3.60 (br s, 1H), 3.13-3.08 (m, 2H), 2.38-2.25 (m, 3H) |
| I-941 | 733.4 | 12.24-12.08 (m, 1H), 8.20-8.01 (m, 2H), 7.76-7.59 (m, 1H), 7.47-7.34 (m, |
| | | 3H), 7.19-7.12 (m, 1H), 7.06 (t, J = 7.2 Hz, 1H), 7.00-6.90 (m, 1H), 6.47- |
| | | 6.41 (m, 1H), 6.17-6.09 (m, 1H), 4.68-4.59 (m, 2H), 4.42-4.32 (m, 2H), 3.89 |
| | | (s, 3H), 3.80 (br d, J = 1.2 Hz, 5H), 3.74 (d, J = 3.6 Hz, 3H), 3.66 (br t, J = 6.0 |
| | | Hz, 1H), 3.60-3.54 (m, 4H), 3.15-3.09 (m, 2H), 2.38-2.27 (m, 3H) |
| I-942 | 702.5 | 12.24-12.13 (m, 1H), 8.17-8.08 (m, 1H), 7.73-7.65 (m, 1H), 7.47-7.33 (m, |
| | | 4H), 7.20-7.03 (m, 4H), 7.01-6.88 (m, 1H), 6.50-6.44 (m, 1H), 6.19-6.11 |
| | | (m, 1H), 4.68-4.58 (m, 2H), 4.42-4.29 (m, 2H), 4.11 (q, J = 7.2 Hz, 2H), 3.89 |
| | | (br s, 4H), 3.78-3.72 (m, 3H), 3.70-3.58 (m, 2H), 3.27 (br s, 4H), 3.10 (q, J = |
| | | 6.8 Hz, 2H), 2.40-2.25 (m, 2H), 1.35 (t, J = 7.2 Hz, 3H) |
| I-943 | 728.3 | 12.25-12.07 (m, 1H), 8.19-8.07 (m, 1H), 7.75-7.62 (m, 1H), 7.53-7.30 (m, |
| | | 4H), 7.20-7.03 (m, 4H), 7.01-6.90 (m, 1H), 6.47 (br s, 1H), 6.19-6.10 (m, |
| | | 1H), 4.69-4.59 (m, 2H), 4.42-4.30 (m, 2H), 3.99 (d, J = 6.8 Hz, 2H), 3.88 (br |
| | | s, 4H), 3.77-3.70 (m, 3H), 3.69-3.57 (m, 2H), 3.25 (br s, 4H), 3.10 (q, J = 6.8 |
| | | Hz, 2H), 2.41-2.24 (m, 2H), 1.32-1.15 (m, 1H), 0.54-0.36 (m, 4H). |
| I-944 | 701.3 | |
| I-945 | 702.3 | |
| I-946 | 676.3 | |
| I-947 | 751.3 | |
| I-948 | 752.3 | |
| I-949 | 696.3 | |
| I-950 | 689.3 | 12.17 (br s, 1H), 8.44-8.24 (m, 2H), 8.19-7.96 (m, 1H), 7.77-7.62 (m, 1H), |
| | | 7.45-7.31 (m, 2H), 7.16 (br d, J = 8.2 Hz, 1H), 7.06 (t, J = 7.4 Hz, 1H), 7.00- |
| | | 6.89 (m, 1H), 6.75 (s, 1H), 6.48 (br s, 1H), 6.20-6.08 (m, 1H), 4.67-4.57 (m, |
| | | 2H), 4.42-4.28 (m, 2H), 3.90 (br s, 4H), 3.77-3.72 (m, 3H), 3.70-3.52 (m, |
| | | 6H), 3.10 (q, J = 6.7 Hz, 2H), 2.42-2.21 (m, 5H) |
| I-951 | 676.3 | |
| I-952 | 701 | |
| I-953 | 703.3 | |
| I-954 | 686.3 | |
| I-955 | 732.3 | |
| I-956 | 714.3 | |
| I-957 | 714.3 | 12.18 (br d, J = 7.1 Hz, 1H), 8.14-8.05 (m, 2H), 7.79 (d, J = 8.1 Hz, 1H), 7.73- |
| | | 7.67 (m, 1H), 7.48-7.33 (m, 4H), 7.16 (br d, J = 8.2 Hz, 1H), 7.09-7.02 (m, |
| | | 2H), 7.01-6.89 (m, 1H), 6.75 (d, J = 2.2 Hz, 1H), 6.48 (br s, 1H), 6.17-6.11 |
| | | (m, 1H), 4.69-4.58 (m, 2H), 4.39 (br s, 1H), 4.36-4.30 (m, 1H), 4.26-3.86 (m, |
| | | 4H), 3.85 (s, 3H), 3.76-3.71 (m, 3H), 3.69-3.57 (m, 2H), 3.20-2.91 (m, 6H), |
| | | 2.39-2.24 (m, 2H) |
| I-958 | 693.3 | |
| I-959 | 688.3 | |
| I-960 | 664.3 | |
| I-961 | 774.4 | 0.67 (br s, 2 H) 0.79 (q, J = 6.0 Hz, 2 H) 1.51 (br d, J = 4.8 Hz, 2 H) 1.62 (br d, |
| | | J = 3.2 Hz, 4 H) 2.19-2.39 (m, 2 H) 2.99-3.13 (m, 10 H) 3.56-3.61 (m, 1 H) |
| | | 3.62-3.68 (m, 1 H) 3.65 (br t, J = 5.6 Hz, 1 H) 3.68-3.79 (m, 7 H) 3.91 (dt, |
| | | J = 5.6, 3.2 Hz, 1 H) 4.19-4.46 (m, 2 H) 4.57-4.73 (m, 2 H) 6.06-6.18 (m, 1 H) |
| | | 6.40 (br s, 1 H) 6.89 (d, J = 6.4 Hz, 1 H) 6.95 (br d, J = 6.0 Hz, 1 H) 7.04 (t, J = 7.2 |
| | | Hz, 1 H) 7.13-7.24 (m, 2 H) 7.31-7.41 (m, 2 H) 7.47 (d, J = 2.4 Hz, 1 H) 7.59- |
| | | 7.74 (m, 1 H) 8.01-8.22 (m, 1 H) 12.09 (br d, J = 6.4 Hz, 1 H) |
| I-962 | 719 | |
| I-963 | 674 | |
| I-964 | 756.2 | |
| I-965 | 700.1 | |
| I-966 | 674.3 | |
| I-967 | 701.3 | 12.24-12.08 (m, 1H), 8.11 (d, J = 9.2 Hz, 1H), 7.69 (d, J = 10.8 Hz, 1H), 7.42- |
| | | 7.32 (m, 4H), 7.18-7.12 (m, 1H), 7.08-7.00 (m, 2H), 6.99-6.89 (m, 2H), |
| | | 6.46 (s, 1H), 6.13 (dd, J = 2.0, 7.2 Hz, 1H), 5.96 (s, 1H), 4.67-4.59 (m, 2H), |
| | | 4.40-4.31 (m, 2H), 4.11 (dd, J = 1.6, 4.0 Hz, 2H), 3.87 (s, 4H), 3.73 (s, 3H), |
| | | 3.68-3.57 (m, 3H), 3.12-3.07 (m, 2H), 2.99 (s, 3H), 2.38-2.25 (m, 2H), 1.27 |
| | | (s, 3H) |
| I-968 | 702.3 | 12.36-11.70 (m, 1H), 8.26-7.86 (m, 1H), 7.72-7.63 (m, 3H), 7.61-7.56 (m, |
| | | 1H), 7.41-7.32 (m, 3H), 7.14 (d, J = 8.4 Hz, 1H), 7.04 (t, J = 7.2 Hz, 1H), 6.98- |
| | | 6.87 (m, 1H), 6.46 (d, J = 2.4 Hz, 1H), 6.17-6.08 (m, 1H), 4.66-4.58 (m, 2H), |
| | | 4.39-4.29 (m, 2H), 3.80 (s, 4H), 3.71 (d, J = 3.6 Hz, 3H), 3.67-3.58 (m, 2H), |
| | | 3.09 (q, J = 6.8 Hz, 2H), 2.90 (s, 4H), 2.36-2.25 (m, 2H) |
| I-969 | 662.4 | 12.13 (br s, 1H), 8.13-8.08 (m, 1H), 7.71-7.65 (m, 1H), 7.42-7.36 (m, 1H), |
| | | 7.36-7.33 (m, 1H), 7.22 (d, J = 7.2 Hz, 1H), 7.17-7.13 (m, 2H), 7.09-7.02 (m, |
| | | 3H), 6.97-6.89 (m, 1H), 6.44 (d, J = 2.8 Hz, 1H), 6.16-6.10 (m, 1H), 4.65- |
| | | 4.59 (m, 2H), 4.38-4.30 (m, 2H), 3.82 (br s, 4H), 3.73-3.71 (m, 3H), 3.65 (br t, |
| | | J = 5.6 Hz, 1H), 3.60 (br t, J = 5.6 Hz, 1H), 3.12-3.07 (m, 2H), 2.84 (br s, 4H), |
| | | 2.71-2.66 (m, 2H), 2.36-2.26 (m, 2H), 1.19 (t, J = 7.6 Hz, 3H) |
| I-970 | 694.4 | 12.16-12.07 (m, 1H), 8.17-8.01 (m, 1H), 7.81-7.54 (m, 1H), 7.41-7.33 (m, |
| | | 2H), 7.17-7.13 (m, 1H), 7.07-7.02 (m, 1H), 6.98-6.90 (m, 2H), 6.73-6.69 |
| | | (m, 1H), 6.59-6.54 (m, 1H), 6.46-6.42 (m, 1H), 6.17-6.10 (m, 1H), 4.65- |
| | | 4.59 (m, 2H), 4.39-4.31 (m, 2H), 3.83 (s, 4H), 3.76-3.71 (m, 9H), 3.66-3.58 |
| | | (m, 2H), 3.09 (d, J = 7.2 Hz, 2H), 3.03 (s, 4H), 2.36-2.26 (m, 2H) |
| I-971 | 665.3 | |
| I-972 | 703.3 | |
| I-973 | 697.3 | 12.29-12.18 (m, 1H), 8.15-8.08 (m, 1H), 7.78 (dd, J = 1.2, 4.8 Hz, 1H), 7.73- |
| | | 7.66 (m, 1H), 7.36-7.29 (m, 1H), 7.29-7.24 (m, 2H), 7.23-7.18 (m, 1H), 7.06- |
| | | 6.96 (m, 1H), 6.90 (dd, J = 4.8, 7.8 Hz, 1H), 6.51 (br d, J = 3.2 Hz, 1H), 6.21- |
| | | 6.12 (m, 1H), 4.63 (q, J = 7.2 Hz, 3H), 4.42-4.31 (m, 2H), 4.27-4.12 (m, 1H), |
| | | 3.85 (br d, J = 10.8 Hz, 2H), 3.81 (s, 3H), 3.66 (br t, J = 5.6 Hz, 1H), 3.63-3.59 |
| | | (m, 4H), 3.53-3.40 (m, 1H), 3.14-3.05 (m, 2H), 2.86 (br dd, J = 3.2, 12.4 Hz, |
| | | 1H), 2.79-2.70 (m, 1H), 2.37 (br s, 1H), 2.29 (br s, 1H), 1.34 (br d, J = 6.4 Hz, 3H) |
| I-974 | 592.3 | |
| I-975 | 616.2 | |
| I-976 | 637.2 | |
| I-977 | 673.3 | 12.18 (br s, 1H), 8.17-8.05 (m, 1H), 7.75-7.66 (m, 1H), 7.62-7.50 (m, 2H), |
| | | 7.49-7.32 (m, 3H), 7.17 (br d, J = 8.2 Hz, 1H), 7.06 (t, J = 7.5 Hz, 1H), 7.01- |
| | | 6.88 (m, 1H), 6.54 (br s, 1H), 6.20-6.08 (m, 1H), 5.03-4.83 (m, 2H), 4.70- |
| | | 4.56 (m, 2H), 4.43-4.28 (m, 2H), 3.92 (br s, 2H), 3.76 (d, J = 3.4 Hz, 3H), 3.69- |
| | | 3.55 (m, 2H), 3.10 (q, J = 6.7 Hz, 2H), 3.00 (br t, J = 5.6 Hz, 2H), 2.40-2.25 (m, 2H) |
| I-978 | 619.3 | |
| I-979 | 596.2 | |
| I-980 | 650.3 | |
| I-981 | 690.3 | |
| I-982 | 609.2 | |
| I-983 | 586.2 | |
| I-984 | 627.3 | |
| I-985 | 559.2 | |
| I-986 | 608.3 | |
| I-987 | 674.3 | |
| I-988 | 616.3 | |
| I-989 | 636.3 | |
| I-990 | 601.6 | 11.96 (d, J = 8.0 Hz, 1H), 8.14-8.08 (m, 1H), 7.72-7.63 (m, 1H), 7.05 (d, J = |
| | | 8.0 Hz, 1H), 6.85-6.78 (m, 1H), 6.38 (s, 1H), 6.29 (s, 1H), 6.21 (d, J = 7.2 Hz, |
| | | 1H), 6.11-6.06 (m, 1H), 5.84-5.78 (m, 1H), 4.64-4.59 (m, 2H), 4.36-4.28 |
| | | (m, 2H), 3.65 (d, J = 4.4 Hz, 6H), 3.59 (t, J = 5.6 Hz, 2H), 3.09 (q, J = 7.2 Hz, |
| | | 2H), 2.73 (d, J = 4.8 Hz, 3H), 2.32 (s, 6H), 2.26 (d, J = 2.4 Hz, 1H), 2.18 (s, 3H) |
| I-991 | 692.3 | 12.13 (br s, 1H), 8.18-8.04 (m, 1H), 7.75-7.62 (m, 1H), 7.45-7.30 (m, 3H), |
| | | 7.25-7.12 (m, 2H), 7.11-6.87 (m, 4H), 6.45 (br s, 1H), 6.21-6.02 (m, 1H), |
| | | 4.70-4.53 (m, 2H), 4.43-4.27 (m, 2H), 3.84-3.57 (m, 14H), 3.27-3.15 (m, |
| | | 2H), 3.10 (q, J = 7.1 Hz, 2H), 2.40-2.23 (m, 2H) |
| I-992 | 622.3 | |
| I-993 | 569.2 | |
| I-994 | 600.3 | |
| I-995 | 650.3 | |
| I-996 | 586.3 | |
| I-997 | 614.5 | |
| I-998 | 615.3 | |
| I-999 | 639.3 | |
| I-1000 | 615.3 | |
| I-1001 | 611.3 | |
| I-1002 | 648.3 | |
| I-1003 | 651.3 | |
| I-1004 | 695.3 | |
| I-1005 | 663.3 | 2.31-12.11 (m, 1H), 8.21-8.12 (m, 1H), 7.79-7.70 (m, 1H), 7.52 (s, 1H), 7.48- |
| | | 7.38 (m, 2H), 7.21 (br d, J = 8.3 Hz, 1H), 7.14-7.07 (m, 1H), 7.04-6.92 (m, |
| | | 1H), 6.50 (d, J = 2.5 Hz, 1H), 6.19 (br d, J = 10.5 Hz, 1H), 4.73-4.63 (m, 2H), |
| | | 4.47-4.33 (m, 2H), 3.93 (s, 3H), 3.86 (br d, J = 1.2 Hz, 4H), 3.80-3.76 (m, |
| | | 3H), 3.74-3.61 (m, 2H), 3.24 (br s, 4H), 3.15 (q, J = 7.2 Hz, 2H), 2.40 (br s, 2H) |
| I-1006 | 573.3 | |
| I-1007 | 573.3 | |
| I-1008 | 649.3 | |
| I-1009 | 677.2 | 12.19-12.12 (m, 1H), 8.14-8.08 (m, 1H), 7.74 (dd, J = 1.9, 13.2 Hz, 1H), 7.70- |
| | | 7.66 (m, 1H), 7.59 (dd, J = 1.7, 8.3 Hz, 1H), 7.43-7.36 (m, 1H), 7.34 (dd, J = |
| | | 1.6, 7.4 Hz, 1H), 7.19-7.11 (m, 2H), 7.07-7.01 (m, 1H), 6.98-6.87 (m, 1H), |
| | | 6.45 (br d, J = 2.1 Hz, 1H), 6.18-6.08 (m, 1H), 4.68-4.57 (m, 2H), 4.40-4.28 |
| | | (m, 2H), 3.84 (br d, J = 1.8 Hz, 4H), 3.76-3.71 (m, 3H), 3.68-3.56 (m, 2H), |
| | | 3.24 (br s, 4H), 3.09 (q, J = 7.3 Hz, 2H), 2.36-2.25 (m, 2H) |
| I-1010 | 607.3 | |
| I-1011 | 555.2 | |
| I-1012 | 620.3 | |
| I-1013 | 664.3 | |
| I-1014 | 637.3 | |
| I-1015 | 675.3 | |
| I-1016 | 689.3 | |
| I-1017 | 675.3 | |
| I-1018 | 675.3 | |
| I-1019 | 688.3 | |
| I-1020 | 665.3 | 12.15 (br s, 1H), 8.16-8.05 (m, 1H), 7.74-7.63 (m, 1H), 7.43-7.37 (m, 1H), |
| | | 7.37-7.31 (m, 2H), 7.15 (br d, J = 8.4 Hz, 1H), 7.08-7.02 (m, 1H), 6.98-6.88 |
| | | (m, 1H), 6.45 (d, J = 2.8 Hz, 1H), 6.13 (d, J = 9.0 Hz, 2H), 5.93-5.79 (m, 1H), |
| | | 4.67-4.55 (m, 2H), 4.42-4.28 (m, 2H), 3.74-3.70 (m, 3H), 3.65 (br t, J = 5.7 |
| | | Hz, 1H), 3.60 (br t, J = 5.7 Hz, 1H), 3.43 (s, 3H), 3.32 (s, 4H), 3.09 (q, J = 7.0 |
| | | Hz, 2H), 3.05-2.82 (m, 4H), 2.41-2.24 (m, 2H) |
| I-1021 | 665.4 | |
| I-1022 | 665.3 | |
| I-1023 | 679.3 | |
| I-1024 | 655.3 | |
| I-1025 | 638.3 | |
| I-1026 | 638.3 | |
| I-1027 | 596.2 | |
| I-1028 | 609.3 | |
| I-1029 | 596.2 | 12.18 (br d, J = 6.2 Hz, 1H), 8.81-8.54 (m, 1H), 8.20-8.05 (m, 1H), 7.78-7.61 |
| | | (m, 1H), 7.48-7.33 (m, 2H), 7.16 (br d, J = 8.5 Hz, 1H), 7.06 (t, J = 7.5 Hz, 1H), |
| | | 7.00-6.87 (m, 1H), 6.53 (d, J = 2.9 Hz, 1H), 6.14 (br d, J = 11.0 Hz, 1H), 5.77 |
| | | (s, 1H), 4.87-4.70 (m, 2H), 4.69-4.52 (m, 2H), 4.42-4.29 (m, 2H), 3.93 (br s, |
| | | 2H), 3.79-3.71 (m, 3H), 3.68-3.55 (m, 2H), 3.10 (q, J = 7.1 Hz, 2H), 2.91 (br t, |
| | | J = 5.5 Hz, 2H), 2.42-2.21 (m, 2H) |
| I-1030 | 665.3 | |
| I-1031 | 557.2 | |
| I-1032 | 678.3 | |
| I-1033 | 599.3 | |
| I-1034 | 678.3 | |
| I-1035 | 691.4 | 12.02 (d, J = 8.8 Hz, 1H), 8.15-8.06 (m, 1H), 7.71-7.63 (m, 2H), 7.42-7.32 |
| | | (m, 2H), 7.14 (d, J = 8.4 Hz, 1H), 7.10 (dd, J = 1.2, 7.6 Hz, 1H), 7.04 (t, J = 7.2 |
| | | Hz, 1H), 6.97-6.87 (m, 1H), 6.63 (dd, J = 4.8, 7.6 Hz, 1H), 6.58 (s, 1H), 6.18- |
| | | 6.05 (m, 1H), 4.66-4.58 (m, 2H), 4.38-4.29 (m, 2H), 3.99 (d, J = 1.6 Hz, 1H), |
| | | 3.89-3.81 (m, 1H), 3.72 (s, 2H), 3.71 (s, 4H), 3.66-3.57 (m, 3H), 3.55-3.41 |
| | | (m, 4H), 3.09 (q, J = 7.2 Hz, 2H), 3.01-2.91 (m, 2H), 2.38-2.22 (m, 3H) |
| I-1036 | 695.3 | 12.22 (d, J = 8.8 Hz, 1H), 8.47 (d, J = 5.6 Hz, 1H), 8.42 (s, 1H), 8.14-8.08 (m, |
| | | 1H), 7.97 (s, 1H), 7.74-7.64 (m, 1H), 7.19 (d, J = 6.0 Hz, 1H), 7.07-6.99 (m, |
| | | 1H), 6.53 (s, 1H), 6.16 (d, J = 10.4 Hz, 1H), 4.62 (q, J = 7.6 Hz, 2H), 4.39-4.32 |
| | | (m, 2H), 4.19-4.14 (m, 2H), 3.93 (s, 3H), 3.81 (s, 2H), 3.69-3.56 (m, 4H), 3.10 |
| | | (s, 2H), 3.04 (s, 2H), 2.44 (s, 3H), 2.36-2.26 (m, 2H), 1.23-1.17 (m, 3H), 1.01 |
| | | (d, J = 6.4 Hz, 2H) |
| I-1037 | 750.1 | 9.54-9.38 (m, 1H), 8.89 (d, J = 5.2 Hz, 1H), 8.32 (d, J = 8.4 Hz, 1H), 8.15 (d, J = |
| | | 8.4 Hz, 1H), 7.85-7.71 (m, 3H), 7.70-7.58 (m, 2H), 7.16 (dd, J = 4.1, 5.9 Hz, |
| | | 1H), 7.05-7.00 (m, 1H), 6.72-6.62 (m, 1H), 6.26-6.16 (m, 1H), 4.81 (dt, J = |
| | | 2.0, 6.0 Hz, 2H), 4.52 (s, 1H), 4.34 (s, 1H), 4.07-3.92 (m, 4H), 3.86 (s, 3H), |
| | | 3.80 (t, J = 6.0 Hz, 1H), 3.63-3.56 (m, 4H), 3.43 (s, 4H), 3.09 (t, J = 6.0 Hz, |
| | | 2H), 2.40-2.38 (m, 1H), 1.28-1.24 (m, 1H) |
| I-1038 | 716.5 | 9.56 (d, J = 6.0 Hz, 1H), 8.90 (d, J = 5.6 Hz, 1H), 8.37-8.26 (m, 1H), 8.18 (d, J = |
| | | 8.4 Hz, 1H), 7.90-7.71 (m, 2H), 7.70-7.55 (m, 2H), 7.42 (t, J = 8.0 Hz, 1H), |
| | | 7.17 (d, J = 5.6 Hz, 1H), 6.69 (d, J = 7.2 Hz, 1H), 6.25-6.09 (m, 3H), 4.85-4.76 |
| | | (m, 2H), 4.54-4.31 (m, 2H), 3.99 (s, 4H), 3.85 (s, 3H), 3.80 (t, J = 6.0 Hz, 1H), |
| | | 3.64 (d, J = 4.4 Hz, 4H), 3.62-3.55 (m, 4H), 3.09 (t, J = 6.0 Hz, 2H), 2.40 (s, |
| | | 1H), 1.26 (s, 1H). |
| I-1039 | 742.0 | 12.01 (br d, J = 7.2 Hz, 1H), 8.13-8.09 (m, 1H), 7.82 (d, J = 2.0 Hz, 1H), 7.70- |
| | | 7.66 (m, 1H), 7.39 (d, J = 2.0 Hz, 1H), 7.15 (d, J = 8.4 Hz, 1H), 6.89-6.82 (m, |
| | | 1H), 6.45 (br s, 1H), 6.42 (s, 1H), 6.39 (dd, J = 2.4, 8.4 Hz, 1H), 6.13-6.08 (m, |
| | | 1H), 4.64-4.60 (m, 2H), 4.36-4.29 (m, 2H), 3.84 (s, 3H), 3.80 (br s, 4H), 3.72- |
| | | 3.71 (m, 3H), 3.65 (br t, J = 5.6 Hz, 1H), 3.59 (s, 1H), 3.36-3.34 (m, 4H), 3.09 |
| | | (br d, J = 7.6 Hz, 2H), 2.97 (s, 6H), 2.34 (br d, J = 3.2 Hz, 1H), 2.27 (br s, 1H) |
| I-1040 | 732.2 | 12.33 (d, J = 4.8 Hz, 1H), 8.54 (d, J = 4.4 Hz, 1H), 8.42 (s, 1H), 8.15-8.06 (m, |
| | | 2H), 7.73-7.63 (m, 1H), 7.47-7.42 (m, 2H), 7.00-6.85 (m, 1H), 6.37 (s, 1H), |
| | | 6.17 (s, 1H), 4.63 (q, J = 6.8 Hz, 2H), 4.43-4.32 (m, 2H), 3.89 (s, 3H), 3.81- |
| | | 3.74 (m, 4H), 3.67-3.59 (m, 2H), 3.55 (d, J = 1.2 Hz, 4H), 3.13-3.08 (m, 2H), |
| | | 2.40-2.32 (m, 4H), 1.02 (t, J = 7.2 Hz, 3H) |
| I-1041 | 695.3 | 12.21 (d, J = 8.8 Hz, 1H), 8.49-8.41 (m, 2H), 8.17-8.05 (m, 1H), 7.75-7.60 |
| | | (m, 2H), 7.19 (d, J = 5.6 Hz, 1H), 7.08-6.97 (m, 1H), 6.54 (s, 1H), 6.21-6.11 |
| | | (m, 1H), 4.63 (q, J = 7.2 Hz, 2H), 4.40-4.32 (m, 2H), 4.20-4.14 (m, 2H), 3.90 |
| | | (s, 3H), 3.80 (s, 4H), 3.68-3.59 (m, 2H), 3.39 (s, 4H), 3.14-3.08 (m, 2H), 2.36 |
| | | (d, J = 3.2 Hz, 2H), 2.29 (s, 3H), 1.25-1.18 (m, 3H) |
| I-1042 | 660.3 | 12.18 (br d, J = 7.6 Hz, 1H), 8.54 (br d, J = 3.6 Hz, 1H), 8.17-8.06 (m, 1H), |
| | | 7.77 (dt, J = 2.0, 7.6 Hz, 1H), 7.72-7.64 (m, 1H), 7.56 (br d, J = 7.2 Hz, 1H), |
| | | 7.45-7.33 (m, 2H), 7.29-7.20 (m, 1H), 7.20-7.11 (m, 1H), 7.05 (br t, J = 7.2 |
| | | Hz, 1H), 6.99-6.87 (m, 1H), 6.59 (br s, 1H), 6.51 (br s, 1H), 6.14 (br d, J = 10.8 |
| | | Hz, 1H), 4.89-4.51 (m, 4H), 4.48-4.20 (m, 3H), 3.74 (br d, J = 3.2 Hz, 3H), |
| | | 3.69-3.58 (m, 2H), 3.19-2.97 (m, 4H), 2.37-2.26 (m, 2H), 1.49-0.83 (m, 5H) |
| I-1043 | 555.2 | 12.23 (br d, J = 3.2 Hz, 1H), 9.01 (s, 1H), 8.59 (s, 1H), 8.25-7.98 (m, 1H), 7.80- |
| | | 7.51 (m, 1H), 7.17-6.98 (m, 1H), 6.75 (br s, 1H), 6.28-6.11 (m, 1H), 4.62 (q, |
| | | J = 7.2 Hz, 2H), 4.45-4.29 (m, 2H), 3.68-3.55 (m, 2H), 3.15-3.05 (m, 3H), |
| | | 3.02 (s, 3H), 2.40-2.25 (m, 2H), 1.93-1.83 (m, 1H), 1.13 (br s, 2H), 1.02-0.89 |
| | | (m, 2H), 0.79-0.65 (m, 2H), 0.59 (br s, 2H) |
| I-1044 | 629.5 | 12.61-11.91 (m, 1H), 8.54 (d, J = 5.2 Hz, 1H), 8.43 (s, 1H), 7.79 (d, J = 2.4 Hz, |
| | | 1H), 7.49-7.30 (m, 2H), 7.02-6.84 (m, 1H), 6.37 (d, J = 2.8 Hz, 1H), 6.18 (br s, |
| | | 1H), 4.48-4.34 (m, 2H), 3.84 (s, 3H), 3.78 (br s, 4H), 3.71-3.63 (m, 2H), 3.24 (br |
| | | s, 4H), 3.02-2.90 (m, 1H), 2.47-2.38 (m, 2H), 2.37-2.22 (m, 2H), 1.08-0.99 (m, 9H) |
| I-1045 | 552.2 | 7.96 (s, 1H), 7.63 (s, 1H), 7.38-7.12 (m, 2H), 6.90-6.65 (m, 1H), 6.49 (d, J = |
| | | 1.2 Hz, 1H), 6.05 (s, 1H), 4.79 (d, J = 3.6 Hz, 2H), 4.59 (s, 2H), 4.47-4.26 (m, |
| | | 1H), 4.17-4.05 (m, 2H), 3.99 (d, J = 7.2 Hz, 1H), 3.94-3.64 (m, 4H), 3.63- |
| | | 3.37 (m, 2H), 2.37 (d, J = 3.2 Hz, 1H), 2.27 (s, 1H), 2.12-2.06 (m, 3H), 1.38- |
| | | 1.20 (m, 6H), 0.66-0.53 (m, 1H), 0.16 (s, 2H), −0.05-−0.26 (m, 2H) |
| I-1046 | 577.3 | 7.87 (d, J = 4.8 Hz, 1H), 7.71 (d, J = 7.6 Hz, 1H), 7.36-7.14 (m, 2H), 7.11 (d, J = |
| | | 1.2 Hz, 1H), 6.85-6.58 (m, 1H), 6.13-5.97 (m, 1H), 4.78 (d, J = 10.0 Hz, |
| | | 2H), 4.58 (s, 2H), 4.33 (d, J = 19.2 Hz, 1H), 4.10 (q, J = 7.6 Hz, 2H), 4.00 (d, J = |
| | | 7.2 Hz, 1H), 3.78-3.46 (m, 4H), 2.81 (q, J = 7.6 Hz, 2H), 2.35 (d, J = 2.4 Hz, |
| | | 2H), 2.22 (d, J = 6.4 Hz, 3H), 2.12-2.05 (m, 3H), 1.38-1.21 (m, 6H), 0.54- |
| | | 0.39 (m, 1H), 0.17-0.04 (m, 2H), −0.26 (s, 2H) |
| I-1047 | 551.3 | 7.86 (s, 1H), 7.71 (d, J = 7.6 Hz, 1H), 7.30-7.15 (m, 2H), 7.08 (s, 1H), 6.76- |
| | | 6.59 (m, 1H), 6.15-6.02 (m, 1H), 4.77 (s, 2H), 4.57 (s, 2H), 4.32-4.23 (m, |
| | | 1H), 4.18-4.07 (m, 2H), 4.00 (d, J = 6.8 Hz, 1H), 3.84-3.75 (m, 1H), 3.67- |
| | | 3.56 (m, 2H), 3.46 (s, 1H), 2.81 (d, J = 7.6 Hz, 2H), 2.36 (s, 2H), 2.23 (d, J = |
| | | 5.6 Hz, 3H), 2.08 (s, 3H), 1.38-1.24 (m, 6H), 0.69-0.57 (m, 3H) |

TABLE 6
Compounds synthesized via Method 1, coupling of
the corresponding amines and carboxylic acids

| | | | LCMS | |
|---|---|---|---|---|
| | | | (ESI+) | |
| | | | m/z | |
| I-# | Amine | Acid | (M + H)⁺ | ¹H NMR (400 MHz, DMSO-d₆) δ |
| I-1219 | VH | A | 676.4 | 8.10 (s, 1H), 7.84 (d, J = 2.8 Hz, 1H), 7.68 (s, 2H), 7.31- |
| | | | | 7.12 (m, 3H), 6.16-6.02 (m, 1H), 4.78 (s, 2H), 4.66-4.53 |
| | | | | (m, 4H), 4.46-4.26 (m, 1H), 4.16-4.06 (m, 2H), 4.00 (q, J = |
| | | | | 7.2 Hz, 1H), 3.77-3.62 (m, 1H), 3.60-3.38 (m, 3H), 3.13- |
| | | | | 3.02 (m, 2H), 2.55 (s, 3H), 2.48-2.39 (m, 2H), 2.38-2.27 |
| | | | | (m, 2H), 1.38-1.23 (m, 3H), 1.09-1.02 (m, 3H), 0.54- |
| | | | | 0.41 (m, 1H), 0.18-0.07 (m, 2H), −0.24 (s, 2H) |
| I-1210 | NH | VC | 586.2 | 12.31-11.76 (m, 1H), 8.34-8.01 (m, 1H), 7.81-7.59 (m, |
| | | | | 1H), 7.48-7.36 (m, 1H), 7.35-7.28 (m, 1H), 6.99-6.81 |
| | | | | (m, 1H), 6.14-5.93 (m, 1H), 5.20-5.09 (m, 1H), 5.04- |
| | | | | 4.90 (m, 1H), 4.88-4.76 (m, 1H), 4.73-4.51 (m, 3H), 4.41- |
| | | | | 4.24 (m, 2H), 4.22-4.05 (m, 2H), 3.85-3.73 (m, 1H), |
| | | | | 3.70-3.57 (m, 2H), 3.16-3.03 (m, 2H), 2.93-2.74 (m, |
| | | | | 2H), 2.69-2.62 (m, 2H), 2.59-2.56 (m, 1H), 2.36 (s, 4H), |
| | | | | 2.28 (d, J = 2.8 Hz, 1H), 2.01-1.84 (m, 1H), 1.48-1.28 (m, 3H) |
| I-1212 | LZ | VB | 607.2 | 8.34-8.25 (m, 1H), 8.11-8.04 (m, 1H), 8.02-7.63 (m, |
| | | | | 2H), 7.63-7.33 (m, 1H), 7.18 (s, 1H), 7.28 (s, 1H), 7.04- |
| | | | | 6.83 (m, 1H), 6.52-6.39 (m, 1H), 4.80-4.72 (m, 2H), 4.64- |
| | | | | 4.56 (m, 4H), 4.30 (br s, 2H), 3.80 (s, 3H), 3.55 (brt, J = |
| | | | | 5.6 Hz, 2H), 3.08 (br t, J = 6.4 Hz, 2H), 2.67 (br s, 3H), 2.61 |
| | | | | (br s, 2H), 2.38-2.24 (m, 2H), 1.10-1.00 (m, 3H) |
| I-1213 | BO | VB | 695.2 | 8.13-8.01 (m, 2H), 7.78 (d, J = 2.0 Hz, 1H), 7.71-7.63 (m, |
| | | | | 1H), 7.56 (br d, J = 7.6 Hz, 1H), 7.36 (dd, J = 2.0, 10.4 Hz, |
| | | | | 1H), 7.26-7.17 (m, 2H), 6.95-6.72 (m, 1H), 6.48-6.37 |
| | | | | (m, 1H), 4.59 (br t, J = 6.8 Hz, 2H), 4.29 (br s, 2H), 3.83 (s, |
| | | | | 3H), 3.60 (br s, 4H), 3.55 (br d, J = 5.2 Hz, 2H), 3.30-3.15 |
| | | | | (m, 4H), 3.07 (br t, J = 6.8 Hz, 2H), 2.55 (s, 3H), 2.54-2.51 |
| | | | | (m, 2H), 2.38-2.22 (m, 2H), 1.07-0.99 (m, 3H) |
| I-1214 | NH | VB | 621.1 | 8.24 (br s, 1H), 8.12-8.04 (m, 1H), 7.71-7.64 (m, 1H), |
| | | | | 7.63-7.51 (m, 1H), 7.43-7.34 (m, 1H), 7.33-7.10 (m, |
| | | | | 2H), 6.99-6.74 (m, 1H), 6.50-6.38 (m, 1H), 4.78 (br s, |
| | | | | 2H), 4.65-4.52 (m, 4H), 4.30 (br s, 2H), 4.14-3.96 (m, |
| | | | | 2H), 3.58-3.52 (m, 2H), 3.08 (br t, J = 6.8 Hz, 2H), 2.57 |
| | | | | (br s, 3H), 2.55 (br s, 2H), 2.40-2.22 (m, 2H), 1.35 (t, J = |
| | | | | 7.2 Hz, 2H), 1.11-0.99 (m, 3H) |
| I-1224 | UT | A | 650.4 | 8.75 (s, 1H), 8.66 (d, J = 4.8 Hz, 1H), 8.15 (s, 1H), 7.94 (d, |
| | | | | J = 0.8 Hz, 1H), 7.78 (s, 1H), 7.52 (d, J = 4.8 Hz, 1H), 7.32 |
| | | | | (s, 1H), 7.24-7.21 (m, 1H), 4.87 (s, 2H), 4.79-4.66 (m, |
| | | | | 5H), 4.20-4.00 (m, 1H), 3.88 (s, 3H), 3.81-3.70 (m, 1H), |
| | | | | 3.65-3.52 (m, 1H), 3.19-3.10 (m, 3H), 3.02-2.89 (m, |
| | | | | 1H), 2.67 (s, 1H), 2.61-2.42 (m, 2H), 2.25-1.88 (m, 3H), |
| | | | | 1.72-1.57 (m, 1H), 1.14 (t, J = 7.6 Hz, 3H), 0.82-0.71 (m, |
| | | | | 1H), 0.46-0.32 (m, 2H), 0.00 (d, J = 3.6 Hz, 2H) |
| I-1225 | UN | A | 664.4 | 8.11-8.03 (m, 1H), 7.78 (s, 1H), 7.70-7.61 (m, 2H), 7.27- |
| | | | | 7.13 (m, 2H), 7.06 (d, J = 5.2 Hz, 1H), 4.73 (s, 2H), 4.62- |
| | | | | 4.55 (m, 4H), 4.54-4.45 (m, 1H), 3.92 (d, J = 13.6 Hz, 1H), |
| | | | | 3.80-3.68 (m, 3H), 3.65-3.53 (m, 1H), 3.46 (s, 1H), 3.07- |
| | | | | 3.01 (m, 2H), 2.99-2.92 (m, 1H), 2.79-2.71 (m, 1H), 2.55 |
| | | | | (s, 2H), 2.54 (s, 3H), 2.43-2.36 (m, 2H), 2.05-1.99 (m, |
| | | | | 1H), 1.80-1.72 (m, 1H), 1.54-1.38 (m, 1H), 1.02 (dd, J = |
| | | | | 6.0, 7.2 Hz, 3H), 0.67-0.55 (m, 1H), 0.28-0.14 (m, |
| | | | | 2H), −0.15 (d, J = 4.4 Hz, 2H) |
| I-1226 | UR | A | 664.4 | 8.13-8.01 (m, 1H), 7.79 (s, 1H), 7.74-7.60 (m, 2H), 7.29- |
| | | | | 7.14 (m, 2H), 7.06 (d, J = 5.2 Hz, 1H), 4.73 (s, 2H), 4.64- |
| | | | | 4.55 (m, 4H), 4.51 (d, J = 8.0 Hz, 1H), 4.01-3.87 (m, 1H), |
| | | | | 3.80-3.67 (m, 3H), 3.66-3.53 (m, 1H), 3.12-2.86 (m, |
| | | | | 4H), 2.82-2.72 (m, 1H), 2.58-2.53 (m, 4H), 2.46-2.34 |
| | | | | (m, 2H), 2.10-1.85 (m, 2H), 1.82-1.71 (m, 1H), 1.57- |
| | | | | 1.37 (m, 1H), 1.08-0.98 (m, 3H), 0.68-0.53 (m, 1H), 0.30- |
| | | | | 0.12 (m, 2H), −0.15 (s, 2H) |
| I-1229 | UH | A | 662.3 | 8.10 (s, 1H), 7.84 (s, 1H), 7.77-7.63 (m, 2H), 7.27-7.10 |
| | | | | (m, 3H), 6.15-6.04 (m, 1H), 4.74 (s, 2H), 4.65-4.54 (m, |
| | | | | 4H), 4.46-4.27 (m, 1H), 4.16-4.05 (m, 1H), 3.80-3.68 |
| | | | | (m, 3H), 3.68-3.58 (m, 1H), 3.57-3.50 (m, 1H), 3.43 (dd, |
| | | | | J = 7.6, 15.2 Hz, 2H), 3.12-3.03 (m, 2H), 2.54 (d, J = 4.0 |
| | | | | Hz, 3H), 2.48-2.40 (m, 2H), 2.38-2.27 (m, 2H), 1.05 (q, J = |
| | | | | 7.2 Hz, 3H), 0.52-0.40 (m, 1H), 0.19-0.07 (m, 2H), −0.24 |
| | | | | (s, 2H) |
| I-1215 | NH | PZ | 622.3 | 8.40-8.37 (m, 1H), 8.12-8.05 (m, 1H), 7.68 (s, 1H), 7.59 |
| | | | | (dd, J = 5.2, 7.6 Hz, 1H), 7.53 (dd, J = 1.6, 5.6 Hz, 1H), |
| | | | | 7.31-7.14 (m, 2H), 6.99-6.92 (m, 1H), 4.78 (br d, J = 6.4 |
| | | | | Hz, 2H), 4.61-4.55 (m, 4H), 4.41-4.34 (m, 2H), 4.15- |
| | | | | 4.08 (m, 1H), 4.03-3.96 (m, 1H), 3.63-3.55 (m, 2H), 3.10- |
| | | | | 2.99 (m, 2H), 2.55 (s, 3H), 2.43 (br d, J = 1.2 Hz, 2H), |
| | | | | 2.36-2.32 (m, 1H), 1.36 (t, J = 7.2 Hz, 2H), 1.26 (t, J = 7.2 |
| | | | | Hz, 2H), 1.11-1.04 (m, 3H) |
| I-1227 | UA | A | 647.4 | 8.16-8.10 (m, 1H), 7.72-7.60 (m, 3H), 7.21 (d, J = 7.8 Hz, |
| | | | | 1H), 6.86 (s, 1H), 6.81-6.60 (m, 1H), 6.03-5.96 (m, 1H), |
| | | | | 4.62 (t, J = 6.8 Hz, 2H), 4.39-4.25 (m, 1H), 4.09 (d, J = |
| | | | | 17.6 Hz, 1H), 3.78-3.58 (m, 2H), 3.54-3.46 (m, 4H), 3.42 |
| | | | | (d, J = 6.4 Hz, 2H), 3.14-3.04 (m, 2H), 2.67 (s, 4H), 2.55 |
| | | | | (s, 3H), 2.47-2.38 (m, 2H), 2.33-2.19 (m, 2H), 1.68-1.62 |
| | | | | (m, 1H), 1.02 (t, J = 7.6 Hz, 3H), 0.51-0.45 (m, 1H), 0.42 |
| | | | | (d, J = 4.8 Hz, 2H), 0.32 (d, J = 2.0 Hz, 2H), 0.13-0.04 (m, |
| | | | | 2H), −0.18-−0.31 (m, 2H) |

TABLE 7
Compounds synthesized via Method 1, coupling of the corresponding amines and carboxylic acids

| | LCMS (ESI+) | |
|---|---|---|
| I-# | m/z (M + H)⁺ | ¹H NMR (400 MHz, DMSO-d₆) δ |
| I-1206 | 675.3 | |
| I-1207 | 675.3 | |
| I-1245 | 680.3 | 7.79 (s, 1H), 7.68-7.61 (m, 2H), 7.56-7.49 (m, 1H), 7.26-7.15 (m, 2H), 7.06 |
| | | (s, 1H), 4.73 (s, 2H), 4.61-4.49 (m, 3H), 4.04-3.90 (m, 1H), 3.80-3.68 (m, |
| | | 3H), 3.67-3.53 (m, 1H), 3.48-3.41 (m, 1H), 3.24-3.20 (m, 2H), 3.13-3.02 |
| | | (m, 1H), 2.86 (t, J = 7.2 Hz, 2H), 2.80 (d, J = 13.6 Hz, 1H), 2.55 (s, 2H), 2.54 (s, |
| | | 3H), 2.43-2.36 (m, 2H), 2.07 (s, 1H), 1.77 (d, J = 12.4 Hz, 1H), 1.57-1.43 (m, |
| | | 1H), 1.02 (d, J = 7.2 Hz, 3H), 0.68-0.57 (m, 1H), 0.27-0.12 (m, 2H), −0.14-−0.21 |
| | | (m, 2H) |
| I-1244 | 680.4 | 7.79 (s, 1H), 7.71-7.59 (m, 2H), 7.56-7.49 (m, 1H), 7.27-7.14 (m, 2H), 7.06 |
| | | (s, 1H), 4.73 (s, 2H), 4.65-4.48 (m, 3H), 4.07-3.92 (m, 1H), 3.81-3.68 (m, |
| | | 3H), 3.67-3.53 (m, 1H), 3.44 (d, J = 11.2 Hz, 1H), 3.21 (q, J = 7.2 Hz, 2H), |
| | | 3.13-2.98 (m, 1H), 2.89-2.74 (m, 3H), 2.54 (s, 4H), 2.45-2.33 (m, 2H), 2.11- |
| | | 1.71 (m, 3H), 1.61-1.39 (m, 1H), 1.09-0.96 (m, 3H), 0.68-0.55 (m, 1H), 0.30- |
| | | 0.12 (m, 2H), −0.16 (s, 2H) |
| I-1243 | 683.3 | 7.68-7.59 (m, 2H), 7.58-7.49 (m, 2H), 7.21 (d, J = 7.6 Hz, 1H), 6.85 (s, 1H), |
| | | 4.63-4.47 (m, 1H), 4.04-3.91 (m, 1H), 3.69-3.52 (m, 2H), 3.51-3.38 (m, |
| | | 6H), 3.21 (d, J = 7.2 Hz, 2H), 3.08 (t, J = 12.8 Hz, 1H), 2.96-2.71 (m, 4H), 2.67- |
| | | 2.57 (m, 1H), 2.54 (s, 3H), 2.47-2.27 (m, 3H), 2.06-1.84 (m, 2H), 1.80-1.70 |
| | | (m, 1H), 1.67-1.62 (m, 1H), 1.56-1.41 (m, 1H), 1.03-0.97 (m, 3H), 0.66- |
| | | 0.56 (m, 1H), 0.41 (d, J = 4.8 Hz, 2H), 0.32 (d, J = 2.4 Hz, 2H), 0.25-0.11 (m, |
| | | 2H), −0.10-−0.20 (m, 2H) |
| I-1242 | 667.3 | 8.27-8.18 (m, 1H), 7.84-7.75 (m, 2H), 7.70 (s, 1H), 7.40-7.33 (m, 1H), 7.03- |
| | | 6.97 (m, 1H), 4.77-4.63 (m, 3H), 4.12-3.99 (m, 1H), 3.87-3.66 (m, 2H), 3.66- |
| | | 3.50 (m, 6H), 3.22-3.16 (m, 2H), 3.10-2.77 (m, 3H), 2.70 (br s, 3H), 2.55- |
| | | 2.48 (m, 3H), 2.23-2.09 (m, 2H), 1.92-1.78 (m, 1H), 1.71-1.59 (m, 1H), 1.47- |
| | | 1.31 (m, 1H), 1.18-1.12 (m, 3H), 0.80-0.68 (m, 1H), 0.57 (br d, J = 4.8 Hz, |
| | | 2H), 0.47 (br s, 2H), 0.41-0.30 (m, 2H), 0.03-−0.05 (m, 2H) |
| I-1239 | 667.3 | 8.27-8.18 (m, 1H), 7.84-7.75 (m, 2H), 7.70 (s, 1H), 7.40-7.33 (m, 1H), 7.03- |
| | | 6.97 (m, 1H), 4.77-4.63 (m, 3H), 4.12-3.99 (m, 1H), 3.87-3.66 (m, 2H), 3.66- |
| | | 3.50 (m, 6H), 3.22-3.16 (m, 2H), 3.10-2.77 (m, 3H), 2.70 (br s, 3H), 2.55- |
| | | 2.48 (m, 3H), 2.23-2.09 (m, 2H), 1.92-1.78 (m, 1H), 1.71-1.59 (m, 1H), 1.47- |
| | | 1.31 (m, 1H), 1.18-1.12 (m, 3H), 0.80-0.68 (m, 1H), 0.57 (br d, J = 4.8 Hz, |
| | | 2H), 0.47 (br s, 2H), 0.41-0.30 (m, 2H), 0.03-−0.05 (m, 2H) |
| I-1237 | 679.5 | 12.38-12.20 (m, 1H), 8.32 (s, 1H), 8.12 (s, 1H), 7.78 (d, J = 2.5 Hz, 1H), 7.70 |
| | | (s, 1H), 7.40-7.33 (m, 3H), 6.94-6.81 (m, 1H), 6.39 (d, J = 1.8 Hz, 1H), 4.68- |
| | | 4.54 (m, 5H), 3.83 (s, 3H), 3.77 (s, 4H), 3.66-3.58 (m, 2H), 3.24 (s, 4H), 3.21 |
| | | (d, J = 5.1 Hz, 2H), 3.13-3.08 (m, 2H), 2.40-2.28 (m, 3H), 1.25-1.07 (m, 1H), |
| | | 1.01 (t, J = 7.6 Hz, 3H) |
| I-1234 | 449.1 | 12.02-11.95 (m, 1H), 7.97-7.86 (m, 1H), 7.80-7.70 (m, 2H), 7.43-7.29 (m, |
| | | 4H), 7.00-6.89 (m, 1H), 6.88-6.83 (m, 1H), 6.10-6.04 (m, 1H), 4.39-4.33 |
| | | (m, 2H), 3.88-3.83 (m, 2H), 3.68 (q, J = 5.7 Hz, 2H), 3.28-3.01 (m, 6H), 2.28 |
| | | (s, 2H) |
| I-1233 | 463.2 | 12.04-11.88 (m, 1H), 7.91-7.84 (m, 1H), 7.81-7.75 (m, 2H), 7.39 (dd, J = |
| | | 4.4, 8.3 Hz, 1H), 7.34 (d, J = 8.4 Hz, 1H), 7.30 (br d, J = 8.0 Hz, 1H), 7.28-7.23 |
| | | (m, 1H), 7.01-6.94 (m, 1H), 6.87-6.85 (m, 1H), 6.10-6.04 (m, 1H), 4.34- |
| | | 4.28 (m, 2H), 3.66-3.62 (m, 1H), 3.61-3.59 (m, 1H), 3.45 (br d, J = 8.0 Hz, |
| | | 3H), 3.11-3.01 (m, 3H), 2.89 (br d, J = 7.2 Hz, 2H), 2.75 (br t, J = 8.0 Hz, 2H), |
| | | 2.33-2.31 (m, 1H), 2.30-2.23 (m, 1H) |
| I-1232 | 463.1 | 12.02-11.92 (m, 1H), 7.95-7.84 (m, 1H), 7.80-7.72 (m, 1H), 7.72-7.62 (m, |
| | | 1H), 7.44-7.27 (m, 4H), 7.02-6.94 (m, 1H), 6.86 (d, J = 1.6 Hz, 1H), 6.12- |
| | | 6.02 (m, 1H), 4.36-4.27 (m, 2H), 3.68-3.58 (m, 2H), 3.27-3.02 (m, 6H), 2.89 |
| | | (q, J = 7.2 Hz, 2H), 2.79-2.71 (m, 2H), 2.35-2.24 (m, 2H) |
| I-1230 | 453.2 | 12.23-11.62 (m, 1H), 8.18-8.11 (m, 1H), 7.85-7.77 (m, 1H), 7.51 (s, 1H), |
| | | 7.39 (d, J = 8.4 Hz, 1H), 7.05-6.91 (m, 2H), 6.86 (d, J = 3.2 Hz, 1H), 6.17- |
| | | 5.97 (m, 1H), 4.38-4.26 (m, 4H), 3.64 (t, J = 6.0 Hz, 1H), 3.58 (t, J = 5.6 Hz, |
| | | 1H), 3.25-2.95 (m, 8H), 2.35-2.24 (m, 2H) |

### Example I-337 (Method 2): Synthesis of 6-(1-(3-(1H-1,2,3-triazol-1-yl)propanoyl)-1,2,5,6-tetrahydropyridin-3-yl)-7-fluoro-4-(2-methoxyphenyl)-N,N-dimethylbenzo[b]thiophene-2-carboxamide

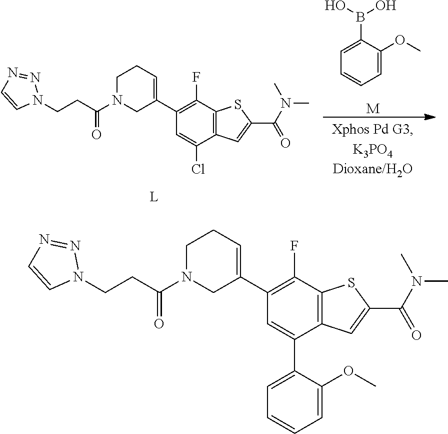

A mixture of 6-(1-(3-(1H-1,2,3-triazol-1-yl)propanoyl)-1,2,5,6-tetrahydropyridin-3-yl)-4-chloro-7-fluoro-N,N-dimethylbenzo[b]thiophene-2-carboxamide (100 mg, 216 μmol, Intermediate L), (2-methoxyphenyl)boronic acid (36.2 mg, 238 μmol, Intermediate M), XPhos Pd G3 (18.3 mg, 21.7 μmol) and K₃PO₄ (138 mg, 649 μmol) in dioxane (2 mL) and H₂O (0.5 mL) was degassed and purged with N₂ three times. Then the mixture was stirred at 80° C. for 1 hr under N₂ atmosphere. On completion, the reaction mixture was concentrated to give a crude residue. The residue was then purified by reversed-phase HPLC (0.8 g/L ammonium bicarbonate) to give the title compound (30.0 mg, 25% yield) as a yellow solid. LC-MS (ESI⁺) m/z 534.2 (M+H)⁺; ¹H NMR (400 MHz, DMSO-d6) δ=8.10 (d, J=11.6 Hz, 1H), 7.68 (d, J=12.0 Hz, 1H), 7.46 (t, J=8.0 Hz, 1H), 7.36-7.25 (m, 3H), 7.18 (br d, J=8.4 Hz, 1H), 7.11-7.05 (m, 1H), 6.28-6.22 (m, 1H), 4.65-4.58 (m, 2H), 4.41-4.34 (m, 2H), 3.71 (d, J=3.2 Hz, 3H), 3.67-3.58 (m, 2H), 3.19-2.99 (m, 8H), 2.37-2.27 (m, 2H).

TABLE 8
Compounds synthesized via Method 2, the cross coupling
of the corresponding chlorides and boronic acids

| | | | LCMS | |
|---|---|---|---|---|
| | | | (ESI+) | |
| | | Boronic | m/z | |
| I-#^{a} | Chloride | Acid | (M + H)⁺ | ¹H NMR (400 MHz, DMSO-d₆) δ |
| I-377^{b} | CE | BZ | 592.3 | 2.0 (s, 1 H), 2.3-2.4 (m, 2 H), 2.7 (br s, 3 H), 3.0-3.1 |
| | | | | (m, 3 H), 3.3-3.3 (m, 6 H), 3.6-3.7 (m, 3 H), 4.2-4.4 |
| | | | | (m, 1 H), 4.4-4.5 (m, 2 H), 4.7-4.9 (m, 1 H), 5.3 (br s, |
| | | | | 1 H), 5.8 (br d, J = 5.2 Hz, 1 H), 6.2-6.3 (m, 3 H), 6.5 |
| | | | | (br s, 1 H), 6.9-7.0 (m, 1 H), 7.0-7.1 (m, 1 H), 7.1- |
| | | | | 7.3 (m, 5 H), 8.2-8.3 (m, 1 H), 8.4 (br s, 1 H), 11.5 (br |
| | | | | d, J = 5.6 Hz, 1 H) |
| I-379 | CI | CG | 437.3 | 2.1 (br d, J = 7.2 Hz, 2 H), 2.2-2.4 (m, 3 H), 3.0-3.1 |
| | | | | (m, 3 H), 3.2 (br s, 3 H), 3.6-3.6 (m, 2 H), 3.9 (s, 3 H), |
| | | | | 4.3-4.4 (m, 2 H), 6.1-6.2 (m, 1 H), 6.5-6.6 (m, 1 H), |
| | | | | 7.0-7.1 (m, 1 H), 7.4-7.4 (m, 1 H), 8.3-8.4 (m, 2 H), |
| | | | | 8.5-8.5 (m, 1 H), 11.6-12.6 (m, 1 H) |
| I-338 | N | B | 559.5 | 12.26-12.15 (m, 1H), 8.27-8.19 (m, 1H), 7.97-7.89 |
| | | | | (m, 1H), 7.86-7.77 (m, 1H), 7.54-7.47 (m, 1H), 7.44- |
| | | | | 7.36 (m, 1H), 7.20-7.01 (m, 2H), 6.96 (s, 1H), 6.27- |
| | | | | 6.14 (m, 1H), 4.74 (q, J = 6.8 Hz, 2H), 4.48-4.37 (m, |
| | | | | 2H), 4.00-3.90 (m, 7H), 3.79-3.69 (m, 2H), 3.49 (s, |
| | | | | 4H), 3.25-3.17 (m, 2H), 2.51-2.34 (m, 2H) |
| I-346 | AK | M | 682.3 | 8.11 (d, J = 11.6 Hz, 1H), 7.80 (dd, J = 1.2, 4.8 Hz, |
| | | | | 1H), 7.69 (d, J = 11.6 Hz, 1H), 7.50-7.44 (m, 1H), |
| | | | | 7.38-7.35 (m, 1H), 7.29-7.26 (m, 3H), 7.20 (d, J = |
| | | | | 8.4 Hz, 1H), 7.10 (t, J = 7.2 Hz, 1H), 6.93 (dd, J = 4.8, |
| | | | | 8.0 Hz, 1H), 6.30-6.24 (m, 1H), 4.65-4.59 (m, 2H), |
| | | | | 4.37 (br d, J = 15.6 Hz, 2H), 3.81 (s, 3H), 3.77 (br s, |
| | | | | 4H), 3.74 (d, J = 2.8 Hz, 3H), 3.63 (td, J = 5.6, 18.8 Hz, |
| | | | | 2H), 3.35 (br s, 4H), 3.10 (q, J = 7.2 Hz, 2H), 2.39- |
| | | | | 2.28 (m, 2H) |
| I-348 | AT | M | 667.4 | 12.03 (br s, 1H), 8.11 (d, J = 11.2 Hz, 1H), 7.80 (d, J = |
| | | | | 4.4 Hz, 1H), 7.70 (d, J = 10.0 Hz, 1H), 7.44-7.36 (m, |
| | | | | 1H), 7.36-7.31 (m, 1H), 7.29-7.24 (m, 1H), 7.18- |
| | | | | 7.13 (m, 1H), 7.09-7.01 (m, 1H), 6.99-6.95 (m, 1H), |
| | | | | 6.94-6.88 (m, 1H), 6.42 (br s, 1H), 4.69-4.57 (m, |
| | | | | 2H), 4.56-4.41 (m, 1H), 3.98-3.85 (m, 1H), 3.85- |
| | | | | 3.76 (m, 7H), 3.73 (s, 3H), 3.33 (s, 4H), 3.29-3.19 (m, |
| | | | | 1H), 3.12-2.98 (m, 3H), 2.80-2.56 (m, 1H), 2.06- |
| | | | | 1.68 (m, 3H), 1.62-1.30 (m, 1H) |
| I-371 | BR | BP | 671.2 | 12.37 (br d, J = 4.8 Hz, 1H), 12.42 (s, 1H), 8.15-8.08 |
| | | | | (m, 1H), 7.84-7.76 (m, 1H), 7.51-7.39 (m, 2H), 7.37- |
| | | | | 7.31 (m, 1H), 7.29-7.24 (m, 1H), 7.05 (s, 1H), 6.92 |
| | | | | (dd, J = 4.8, 7.6 Hz, 1H), 6.65 (br s, 1H), 6.22-6.13 |
| | | | | (m, 1H), 4.65-4.59 (m, 2H), 4.40-4.32 (m, 2H), 3.84- |
| | | | | 3.78 (m, 7H), 3.59 (br s, 2H), 3.35-3.34 (m, 4H), |
| | | | | 3.14-3.09 (m, 2H), 2.38-2.28 (m, 2H) |
| I-390 | BR | CT | 703.3 | 2.24-2.41 (m, 2 H) 2.41-2.52 (m, 11 H) 2.52-2.56 |
| | | | | (m, 2 H) 3.03-3.19 (m, 3 H) 3.51-3.67 (m, 3 H) 3.76 |
| | | | | (br s, 4 H) 3.79-3.86 (m, 3 H) 4.25-4.44 (m, 2 H) |
| | | | | 4.57-4.70 (m, 2 H) 6.08-6.18 (m, 1 H) 6.24-6.38 (m, |
| | | | | 1 H) 6.78-6.85 (m, 1 H) 6.81-6.89 (m, 1 H) 6.92 (dd, |
| | | | | J = 7.6, 4.8 Hz, 1 H) 7.22-7.31 (m, 1 H) 7.48-7.58 (m, |
| | | | | 1 H) 7.61-7.71 (m, 2 H) 7.75-7.82 (m, 2 H) 7.86- |
| | | | | 7.94 (m, 1 H) 8.06-8.15 (m, 1 H) 12.32 (br s, 1 H) |
| I-394 | BR | CX | 683.4 | 12.12 (br d, J = 6.4 Hz, 1H), 8.15-8.08 (m, 1H), 7.80 |
| | | | | (d, J = 3.6 Hz, 1H), 7.74-7.66 (m, 1H), 7.38 (t, J = 7.6 |
| | | | | Hz, 1H), 7.30-7.24 (m, 1H), 7.06 (br d, J = 11.6 Hz, |
| | | | | 1H), 6.98-6.84 (m, 3H), 6.48-6.42 (m, 1H), 6.19- |
| | | | | 6.08 (m, 1H), 4.63 (q, J = 7.2 Hz, 2H), 4.37 (br s, 1H), |
| | | | | 4.34-4.27 (m, 1H), 3.81 (s, 6H), 3.75 (d, J = 3.2 Hz, |
| | | | | 4H), 3.66 (br t, J = 5.2 Hz, 1H), 3.60 (br t, J = 5.6 Hz, |
| | | | | 1H), 3.39-3.34 (m, 4H), 3.10 (q, J = 7.2 Hz, 2H), 2.37- |
| | | | | 2.33 (m, 1H), 2.28 (br d, J = 2.4 Hz, 1H) |
| I-395 | BR | CY | 683.4 | 12.22-12.09 (m, 1H), 8.14-8.05 (m, 1H), 7.79 (dd, J = |
| | | | | 1.2, 4.8 Hz, 1H), 7.72-7.62 (m, 1H), 7.29-7.23 (m, |
| | | | | 1H), 7.21 (d, J = 8.4 Hz, 2H), 7.17-7.11 (m, 1H), 7.02- |
| | | | | 6.85 (m, 2H), 6.47 (s, 1H), 6.20-6.09 (m, 1H), 4.62 |
| | | | | (q, J = 7.2 Hz, 2H), 4.41-4.25 (m, 2H), 3.80 (s, 7H), |
| | | | | 3.71 (d, J = 2.4 Hz, 3H), 3.67-3.58 (m, 2H), 3.36- |
| | | | | 3.33 (m, 4H), 3.09 (q, J = 6.8 Hz, 2H), 2.37-2.25 (m, 2H) |
| I-396 | BR | CZ | 715.4 | 12.33-12.20 (m, 1H), 8.19 (d, J = 7.6 Hz, 1H), 8.14- |
| | | | | 8.08 (m, 1H), 8.02-7.97 (m, 1H), 7.79 (d, J = 8.4 Hz, |
| | | | | 1H), 7.77 (dd, J = 1.2, 4.8 Hz, 1H), 7.71-7.65 (m, |
| | | | | 1H), 7.63-7.56 (m, 3H), 7.25 (dd, J = 1.2, 8.0 Hz, |
| | | | | 1H), 7.21-7.12 (m, 1H), 6.90 (dd, J = 4.8, 8.0 Hz, |
| | | | | 1H), 6.57 (s, 1H), 6.23-6.13 (m, 1H), 4.62 (q, J = 7.2 |
| | | | | Hz, 2H), 4.44-4.34 (m, 2H), 3.78 (s, 7H), 3.69-3.59 |
| | | | | (m, 2H), 3.46-3.42 (m, 3H), 3.31-3.21 (m, 4H), 3.10 |
| | | | | (q, J = 6.8 Hz, 2H), 2.38-2.25 (m, 2H) |
| I-397 | BR | DA | 685.3 | 12.37-12.28 (m, 1H), 8.13-8.07 (m, 1H), 8.01 (t, J = |
| | | | | 8.0 Hz, 2H), 7.78-7.74 (m, 1H), 7.71-7.68 (m, 1H), |
| | | | | 7.68-7.63 (m, 1H), 7.61 (d, J = 8.0 Hz, 1H), 7.57 (d, J = |
| | | | | 6.8 Hz, 1H), 7.53 (d, J = 7.6 Hz, 1H), 7.47-7.41 (m, |
| | | | | 1H), 7.26-7.22 (m, 1H), 7.10-7.00 (m, 1H), 6.89 (dd, |
| | | | | J = 4.8, 8.0 Hz, 1H), 6.24-6.16 (m, 2H), 4.65-4.57 |
| | | | | (m, 2H), 4.43-4.34 (m, 2H), 3.77 (s, 3H), 3.71 (s, 4H), |
| | | | | 3.67-3.63 (m, 1H), 3.60 (t, J = 5.6 Hz, 1H), 3.26 (s, |
| | | | | 4H), 3.12-3.07 (m, 2H), 2.36-2.26 (m, 2H) |
| I-398 | BR | DB | 693.3 | 12.18-12.09 (m, 1H), 8.15-8.09 (m, 1H), 7.80 (dd, J = |
| | | | | 1.2, 4.8 Hz, 1H), 7.72-7.66 (m, 1H), 7.27 (dd, J = |
| | | | | 1.2, 8.0 Hz, 1H), 7.01-6.93 (m, 1H), 6.93-6.90 (m, |
| | | | | 4H), 6.56 (d, J = 2.8 Hz, 1H), 6.18-6.09 (m, 1H), 4.68- |
| | | | | 4.58 (m, 2H), 4.37 (br s, 1H), 4.34-4.27 (m, 3H), |
| | | | | 4.21 (br s, 2H), 3.81 (s, 7H), 3.66 (br t, J = 5.6 Hz, 1H), |
| | | | | 3.60 (br t, J = 5.6 Hz, 1H), 3.40-3.34 (m, 4H), 3.10 (q, |
| | | | | J = 6.8 Hz, 2H), 2.38-2.34 (m, 1H), 2.28 (br s, 1H) |
| I-399^{c} | BR | DC | 683.2 | 8.08-8.00 (m, 1H), 7.75 (dd, J = 1.2, 4.8 Hz, 1H), 7.70- |
| | | | | 7.63 (m, 1H), 7.34-7.12 (m, 4H), 7.02-6.88 (m, |
| | | | | 2H), 6.51 (d, J = 2.8 Hz, 1H), 6.12 (br s, 1H), 4.61 (q, J = |
| | | | | 6.8 Hz, 2H), 4.38-4.26 (m, 2H), 3.77 (s, 7H), 3.62 |
| | | | | (br t, J = 5.6 Hz, 1H), 3.56 (br s, 4H), 3.29 (br s, 4H), |
| | | | | 3.12-3.01 (m, 2H), 2.36-2.19 (m, 2H) |
| I-400 | DG | DF | 635.3 | 12.22 (d, J = 6.8 Hz, 1H), 8.51 (d, J = 5.6 Hz, 1H), 8.41 |
| | | | | (s, 1H), 8.10 (d, J = 11.2 Hz, 1H), 7.99-7.34 (m, 1H), |
| | | | | 7.25-7.20 (m, 3H), 7.03-6.94 (m, 3H), 6.81 (t, J = 7.2 |
| | | | | Hz, 1H), 6.48 (s, 1H), 6.18-6.13 (m, 1H), 4.62 (q, J = |
| | | | | 7.2 Hz, 2H), 4.38-4.31 (m, 2H), 3.83 (d, J = 3.2 Hz, |
| | | | | 7H), 3.68-3.63 (m, 1H), 3.60 (t, J = 5.6 Hz, 1H), 3.19 |
| | | | | (s, 4H), 3.09 (q, J = 6.4 Hz, 2H), 2.36-2.27 (m, 2H) |
| I-402^{c, e} | DD | DE | 716.3 | 12.37 (br s, 1H), 8.87 (s, 1H), 8.28 (d, J = 7.6 Hz, 1H), |
| | | | | 8.14-8.06 (m, 2H), 7.82 (t, J = 7.6 Hz, 1H), 7.78 (dd, J = |
| | | | | 1.2, 4.8 Hz, 1H), 7.70 (s, 1H), 7.69-7.66 (m, 1H), |
| | | | | 7.25 (dd, J = 1.2, 8.0 Hz, 1H), 7.17 (br d, J = 6.0 Hz, |
| | | | | 1H), 6.91 (dd, J = 4.8, 8.0 Hz, 1H), 6.61 (br d, J = 3.2 |
| | | | | Hz, 1H), 6.28-6.19 (m, 1H), 4.63 (q, J = 7.2 Hz, 2H), |
| | | | | 4.47-4.34 (m, 2H), 3.79 (s, 8H), 3.67 (br t, J = 5.6 Hz, |
| | | | | 1H), 3.62 (br t, J = 5.6 Hz, 1H), 3.54 (d, J = 1.6 Hz, |
| | | | | 3H), 3.32-3.28 (m, 3H), 3.15-3.08 (m, 2H), 2.38 (br |
| | | | | s, 1H), 2.30 (br s, 1H) |
| I-403^{c, d} | DH | DJ | 693.3 | 12.29-12.12 (m, 1H), 8.46-8.38 (m, 2H), 8.18-8.00 |
| | | | | (m, 1H), 7.77-7.54 (m, 1H), 7.21-7.17 (m, 1H), 7.04- |
| | | | | 6.94 (m, 3H), 6.90-6.85 (m, 2H), 6.56-6.51 (m, |
| | | | | 1H), 6.20-6.11 (m, 1H), 4.85-4.78 (m, 1H), 4.62 (q, J = |
| | | | | 6.8 Hz, 2H), 4.40-4.31 (m, 2H), 3.81 (s, 4H), 3.78 |
| | | | | (s, 3H), 3.66-3.58 (m, 2H), 3.09 (q, J = 6.4 Hz, 2H), |
| | | | | 2.98 (s, 4H), 2.36-2.26 (m, 2H), 1.27-1.19 (m, 6H) |
| I-404^{c, d} | DK | DJ | 707.3 | 12.18 (d, J = 5.2 Hz, 1H), 8.57-8.25 (m, 2H), 8.15- |
| | | | | 8.01 (m, 1H), 7.74-7.57 (m, 1H), 7.29 (d, J = 5.6 Hz, |
| | | | | 1H), 7.04-6.93 (m, 3H), 6.90-6.85 (m, 2H), 6.55- |
| | | | | 6.49 (m, 1H), 6.20-6.10 (m, 1H), 4.62 (q, J = 6.8 Hz, |
| | | | | 2H), 4.39-4.29 (m, 2H), 3.82 (s, 4H), 3.79 (s, 3H), |
| | | | | 3.67-3.57 (m, 2H), 3.12-3.06 (m, 2H), 2.98 (s, 4H), |
| | | | | 2.36-2.26 (m, 2H), 1.29 (s, 9H) |
| I-405^{c, d} | DL | DJ | 683.2 | 12.36-12.15 (m, 1H), 8.62-8.49 (m, 2H), 8.22-7.91 |
| | | | | (m, 1H), 7.81-7.58 (m, 1H), 7.38 (d, J = 5.6 Hz, 1H), |
| | | | | 7.08-6.95 (m, 3H), 6.90-6.84 (m, 2H), 6.50 (d, J = |
| | | | | 2.0 Hz, 1H), 6.21-6.12 (m, 1H), 6.04-5.87 (m, 2H), |
| | | | | 4.62 (q, J = 7.2 Hz, 2H), 4.40-4.31 (m, 2H), 3.81 (s, |
| | | | | 4H), 3.78 (s, 3H), 3.66-3.58 (m, 2H), 3.10 (q, J = 6.4 |
| | | | | Hz, 2H), 2.98 (s, 4H), 2.36-2.25 (m, 2H) |
| I-406^{c, d} | DM | DJ | 679.3 | 12.32-12.16 (m, 1H), 8.50-8.41 (m, 2H), 8.10 (d, J = |
| | | | | 11.2 Hz, 1H), 7.73-7.58 (m, 1H), 7.18 (d, J = 5.4 Hz, |
| | | | | 1H), 7.06-6.94 (m, 3H), 6.90-6.84 (m, 2H), 6.52 (s, |
| | | | | 1H), 6.20-6.10 (m, 1H), 4.65-4.57 (m, 2H), 4.39- |
| | | | | 4.31 (m, 2H), 4.19-4.13 (m, 2H), 3.81 (s, 4H), 3.78 |
| | | | | (s, 3H), 3.65-3.58 (m, 2H), 3.12-3.07 (m, 2H), 2.98 |
| | | | | (s, 4H), 2.36-2.28 (m, 2H), 1.23-1.16 (m, 3H) |
| I-409 | DP | DF | 584.2 | 12.27-12.09 (m, 1H), 8.50 (d, J = 6.0 Hz, 1H), 8.43- |
| | | | | 8.38 (m, 1H), 7.25-7.15 (m, 1H), 6.99-6.85 (m, 5H), |
| | | | | 6.48 (d, J = 2.8 Hz, 1H), 6.20-6.08 (m, 1H), 4.38- |
| | | | | 4.31 (m, 2H), 3.83 (s, 6H), 3.79 (s, 3H), 3.64-3.58 (m, |
| | | | | 2H), 2.98 (br s, 4H), 2.41-2.32 (m, 2H), 2.30-2.23 |
| | | | | (m, 1H), 2.08 (d, J = 6.0 Hz, 3H) |
| I-410 | BR | DQ | 666.4 | 12.20 (d, J = 5.6 Hz, 1H), 8.25-8.22 (m, 1H), 8.13- |
| | | | | 8.08 (m, 1H), 7.82-7.78 (m, 2H), 7.71-7.65 (m, 1H), |
| | | | | 7.27 (dd, J = 0.8, 8.0 Hz, 1H), 7.12 (dd, J = 5.2, 7.2 Hz, |
| | | | | 1H), 7.05-6.97 (m, 1H), 6.92 (dd, J = 4.8, 8.0 Hz, |
| | | | | 1H), 6.51 (s, 1H), 6.17-6.11 (m, 1H), 4.62 (q, J = 7.2 |
| | | | | Hz, 2H), 4.39-4.31 (m, 2H), 3.84 (d, J = 4.0 Hz, 4H), |
| | | | | 3.80 (s, 6H), 3.65 (t, J = 5.6 Hz, 1H), 3.60 (t, J = 5.6 |
| | | | | Hz, 1H), 3.36-3.32 (m, 4H), 3.09 (q, J = 7.2 Hz, 2H), |
| | | | | 2.36-2.27 (m, 2H) |
| I-411 | BR | DR | 699.3 | 12.27 (d, J = 2.8 Hz, 1H), 8.15-8.08 (m, 1H), 7.79 |
| | | | | (dd, J = 1.2, 4.8 Hz, 1H), 7.72-7.64 (m, 1H), 7.58- |
| | | | | 7.49 (m, 1H), 7.41 (ddd, J = 1.8, 3.6, 5.6 Hz, 1H), 7.28- |
| | | | | 7.22 (m, 2H), 7.10-7.01 (m, 1H), 6.91 (dd, J = 4.8, |
| | | | | 8.0 Hz, 1H), 6.54-6.48 (m, 1H), 6.20-6.11 (m, 1H), |
| | | | | 4.62 (q, J = 7.2 Hz, 2H), 4.40-4.32 (m, 2H), 3.80 (s, |
| | | | | 8H), 3.67-3.58 (m, 2H), 3.38-3.34 (m, 6H), 3.09 (q, |
| | | | | J = 7.6 Hz, 2H), 2.37-2.26 (m, 2H) |
| I-412 | BR | DS | 679.3 | 12.14-12.09 (m, 1H), 8.15-8.05 (m, 1H), 7.79 (dd, J = |
| | | | | 1.2, 4.8 Hz, 1H), 7.76-7.59 (m, 1H), 7.39-7.34 (m, |
| | | | | 2H), 7.26 (dd, J = 1.2, 8.0 Hz, 1H), 7.14 (d, J = 8.0 Hz, |
| | | | | 1H), 7.06-6.96 (m, 2H), 6.92 (dd, J = 4.8, 8.0 Hz, 1H), |
| | | | | 6.49 (d, J = 2.4 Hz, 1H), 6.14 (d, J = 9.6 Hz, 1H), 4.65- |
| | | | | 4.59 (m, 2H), 4.39-4.31 (m, 2H), 4.06-4.00 (m, 2H), |
| | | | | 3.80 (s, 7H), 3.65 (t, J = 5.6 Hz, 1H), 3.60 (t, J = 5.6 |
| | | | | Hz, 1H), 3.35-3.32 (m, 4H), 3.12-3.06 (m, 2H), 2.37- |
| | | | | 2.26 (m, 2H), 1.20-1.12 (m, 3H) |
| I-551^{b} | CE | BZ | 562.4 | 2.2-2.4 (m, 2 H), 2.7 (br s, 3 H), 3.1 (br d, J = 19.6 Hz, |
| | | | | 7 H), 3.7 (br s, 4 H), 4.2-4.4 (m, 1 H), 4.4-4.6 (m, 2 |
| | | | | H), 4.8 (br d, J = 15.2 Hz, 1 H), 5.4 (br s, 1 H), 5.8 (br s, |
| | | | | 1 H), 6.3 (br s, 1 H), 6.7 (br s, 2 H), 6.9 (br s, 1 H), 7.1- |
| | | | | 7.3 (m, 5 H), 7.1-7.2 (m, 1 H), 7.3 (br s, 1 H), 7.4-7.5 |
| | | | | (m, 2 H), 8.3 (br d, J = 13.2 Hz, 1 H), 8.4 (br s, 1 H), |
| | | | | 11.6 (br s, 1 H) |
| I-552^{c, d} | DV | K | 453.0 | 2.07 (d, J = 7.6 Hz, 3 H) 2.24-2.40 (m, 2 H) 2.95- |
| | | | | 3.22 (m, 6 H) 3.57-3.65 (m, 2 H) 3.72 (s, 3 H) 4.35 (br |
| | | | | s, 2 H) 6.25 (br s, 1 H) 7.05-7.12 (m, 1 H) 7.18 (d, J = |
| | | | | 8.4 Hz, 1 H) 7.24-7.37 (m, 3 H) 7.46 (t, J = 7.2 Hz, 1 H) |
| I-553 | DX | DW | 584.4 | 12.17-12.10 (m, 1H), 7.82-7.77 (m, 1H), 7.42-7.38 |
| | | | | (m, 1H), 7.38-7.34 (m, 1H), 7.28-7.24 (m, 1H), 7.18- |
| | | | | 7.13 (m, 1H), 7.09-7.03 (m, 1H), 6.97-6.89 (m, |
| | | | | 2H), 6.48-6.40 (m, 1H), 6.18-6.09 (m, 1H), 4.36- |
| | | | | 4.31 (m, 2H), 3.84-3.76 (m, 8H), 3.73 (s, 3H), 3.65- |
| | | | | 3.59 (m, 2H), 2.52-2.51 (m, 3H), 2.37 (br d, J = 3.2 |
| | | | | Hz, 1H), 2.31-2.22 (m, 1H), 2.08 (s, 2H), 2.07 (s, 1H) |
| I-554 | EA | DZ | 454.1 | 8.53 (s, 1H), 8.38-8.28 (m, 1H), 7.45-7.33 (m, 3H), |
| | | | | 6.31-6.24 (m, 1H), 4.35 (s, 2H), 3.85 (s, 3H), 3.65- |
| | | | | 3.56 (m, 2H), 3.18 (s, 3H), 3.01 (s, 3H), 2.39 (d, J = 3.6 |
| | | | | Hz, 1H), 2.27 (d, J = 3.6 Hz, 1H), 2.07 (d, J = 8.0 Hz, 3H) |
| I-555 | EA | DF | 454.2 | 8.56 (s, 1H), 8.48-8.32 (m, 1H), 7.52-7.28 (m, 2H), |
| | | | | 7.24 (d, J = 5.6 Hz, 1H), 6.32-6.23 (m, 1H), 4.36 (s, |
| | | | | 2H), 3.81 (s, 3H), 3.65-3.57 (m, 2H), 3.21-2.94 (m, |
| | | | | 6H), 2.39 (d, J = 3.6 Hz, 1H), 2.34-2.23 (m, 1H), 2.08 |
| | | | | (d, J = 6.4 Hz, 3H) |
| I-556^{c, d} | EY | DE | 692.5 | 12.19 (d, J = 8.0 Hz, 1H), 8.53 (d, J = 5.6 Hz, 1H), 8.44 |
| | | | | (s, 1H), 8.11 (d, J = 11.2 Hz, 1H), 7.79 (d, J = 4.0 Hz, |
| | | | | 1H), 7.72-7.64 (m, 1H), 7.50-7.43 (m, 1H), 7.27 (d, J = |
| | | | | 8.0 Hz, 1H), 7.02-6.88 (m, 2H), 6.47 (s, 1H), 6.21- |
| | | | | 6.08 (m, 1H), 4.62 (q, J = 7.2 Hz, 2H), 4.40-4.28 (m, |
| | | | | 2H), 4.01-3.94 (m, 1H), 3.80 (s, 7H), 3.65 (t, J = 5.6 |
| | | | | Hz, 1H), 3.60 (t, J = 5.6 Hz, 1H), 3.37-3.33 (m, 4H), |
| | | | | 3.09 (q, J = 6.8 Hz, 2H), 2.37-2.26 (m, 2H), 0.80 (t, J = |
| | | | | 6.4 Hz, 2H), 0.63 (d, J = 8.4 Hz, 2H) |
| I-557 | FA | B | 657.3 | 12.05-12.01 (m, 1H), 8.14-8.09 (m, 1H), 7.81 (dd, J = |
| | | | | 1.2, 4.8 Hz, 1H), 7.78-7.58 (m, 1H), 7.28 (dd, J = |
| | | | | 1.2, 8.0 Hz, 1H), 6.93 (dd, J = 4.8, 8.0 Hz, 1H), 6.72 (s, |
| | | | | 1H), 6.34-6.28 (m, 1H), 6.06 (d, J = 4.0 Hz, 1H), 4.62 |
| | | | | (q, J = 6.4 Hz, 2H), 4.34-4.26 (m, 2H), 3.82 (s, 4H), |
| | | | | 3.64 (t, J = 5.6 Hz, 1H), 3.58 (t, J = 5.6 Hz, 2H), 3.36 |
| | | | | (s, 6H), 3.12-3.04 (m, 7H), 2.55 (s, 4H), 2.33 (s, 1H), |
| | | | | 2.26 (s, 3H) |
| I-558 | BR | FC | 719.3 | 12.31 (s, 1H), 8.13-8.07 (m, 1H), 7.79 (d, J = 4.4 Hz, |
| | | | | 1H), 7.70-7.62 (m, 2H), 7.54 (quin, J = 6.4 Hz, 3H), |
| | | | | 7.27 (d, J = 8.0 Hz, 1H), 7.05-6.96 (m, 1H), 6.92 (dd, |
| | | | | J = 4.8, 8.0 Hz, 1H), 6.49 (d, J = 2.4 Hz, 1H), 6.17- |
| | | | | 6.11 (m, 1H), 4.62 (q, J = 6.8 Hz, 2H), 4.38-4.31 (m, |
| | | | | 2H), 3.82-3.75 (m, 8H), 3.65 (t, J = 5.6 Hz, 1H), 3.60 |
| | | | | (t, J = 5.6 Hz, 1H), 3.32 (s, 3H), 3.12-3.06 (m, 2H), |
| | | | | 2.37-2.27 (m, 2H) |
| I-559 | BR | FD | 701.3 | 12.17 (d, J = 5.6 Hz, 1H), 8.16-8.04 (m, 1H), 7.79 (d, |
| | | | | J = 4.4 Hz, 1H), 7.73-7.64 (m, 1H), 7.49-7.39 (m, |
| | | | | 1H), 7.33-7.23 (m, 2H), 7.00-6.88 (m, 2H), 6.48 (s, |
| | | | | 1H), 6.20-6.07 (m, 1H), 4.62 (q, J = 6.9 Hz, 2H), 4.41- |
| | | | | 4.27 (m, 2H), 3.80 (s, 6H), 3.73 (s, 3H), 3.65 (t, J = |
| | | | | 5.2 Hz, 1H), 3.59 (t, J = 5.6 Hz, 1H), 3.36-3.32 (m, |
| | | | | 5H), 3.09 (q, J = 7.2 Hz, 2H), 2.36-2.25 (m, 2H) |
| I-560 | BR | FE | 679.4 | 12.22-12.15 (m, 1H), 8.14-8.08 (m, 1H), 7.79 (d, J = |
| | | | | 4.4 Hz, 1H), 7.72-7.66 (m, 1H), 7.26 (br dd, J = 3.6, |
| | | | | 6.8 Hz, 3H), 7.16-7.09 (m, 1H), 7.08-7.00 (m, 1H), |
| | | | | 6.91 (dd, J = 4.8, 7.6 Hz, 1H), 6.51 (br s, 1H), 6.18- |
| | | | | 6.10 (m, 1H), 4.62 (q, J = 7.2 Hz, 2H), 4.40-4.30 (m, |
| | | | | 2H), 3.80 (s, 7H), 3.68-3.58 (m, 2H), 3.32 (s, 4H), |
| | | | | 3.23 (d, J = 2.4 Hz, 3H), 3.09 (q, J = 7.2 Hz, 2H), 2.35 |
| | | | | (br d, J = 2.4 Hz, 1H), 2.31 (s, 3H), 2.28 (br s, 1H) |
| I-561^{c, d} | BR | FG | 664.2 | 12.42-12.22 (m, 1H), 8.53 (d, J = 5.2 Hz, 1H), 8.41 (s, |
| | | | | 1H), 8.14-8.01 (m, 1H), 7.81-7.73 (m, 1H), 7.72- |
| | | | | 7.55 (m, 1H), 7.42 (d, J = 5.2 Hz, 1H), 7.26 (dd, J = |
| | | | | 1.2, 8.0 Hz, 1H), 6.98-6.86 (m, 2H), 6.37-6.31 (m, |
| | | | | 1H), 6.21-6.12 (m, 1H), 4.62 (q, J = 6.8 Hz, 2H), 4.42- |
| | | | | 4.31 (m, 2H), 3.80 (s, 3H), 3.77 (s, 4H), 3.67-3.58 |
| | | | | (m, 2H), 3.33-3.31 (m, 6H), 3.12-3.06 (m, 2H), 2.36- |
| | | | | 2.24 (m, 2H), 1.01 (t, J = 7.8 Hz, 3H) |
| I-562 | BR | FH | 663.3 | 12.24 (s, 1H), 8.14-8.06 (m, 1H), 7.78 (dd, J = 1.2, 4.8 |
| | | | | Hz, 1H), 7.71-7.65 (m, 1H), 7.41-7.33 (m, 2H), 7.30- |
| | | | | 7.22 (m, 3H), 6.94-6.77 (m, 2H), 6.29 (s, 1H), 6.19- |
| | | | | 6.10 (m, 1H), 4.62 (q, J = 7.2 Hz, 2H), 4.40-4.30 (m, |
| | | | | 2H), 3.79 (s, 3H), 3.76 (s, 4H), 3.66-3.57 (m, 2H), |
| | | | | 3.31-3.27 (m, 6H), 3.09 (d, J = 5.6 Hz, 2H), 2.36- |
| | | | | 2.24 (m, 2H), 0.97 (t, J = 7.6 Hz, 3H) |
| I-563 | FI | DF | 683.2 | 12.22 (d, J = 7.2 Hz, 1H), 8.50 (d, J = 5.6 Hz, 1H), 8.42 |
| | | | | (s, 1H), 8.13-8.07 (m, 1H), 7.86-7.52 (m, 1H), 7.21 |
| | | | | (d, J = 5.6 Hz, 1H), 7.05-7.01 (m, 1H), 7.00-6.95 (m, |
| | | | | 1H), 6.91-6.85 (m, 1H), 6.77 (d, J = 8.0 Hz, 1H), 6.49 |
| | | | | (s, 1H), 6.16 (d, J = 11.2 Hz, 1H), 4.62 (q, J = 7.2 Hz, |
| | | | | 2H), 4.39-4.31 (m, 2H), 3.86-3.82 (m, 10H), 3.65 (t, |
| | | | | J = 5.2 Hz, 1H), 3.60 (t, J = 5.2 Hz, 1H), 3.12-3.06 |
| | | | | (m, 6H), 2.36-2.26 (m, 2H) |
| I-564 | FP | FO | 680.3 | 12.29-12.09 (m, 1H), 8.50 (d, J = 5.6 Hz, 1H), 8.42 (d, |
| | | | | J = 1.6 Hz, 1H), 8.14-8.04 (m, 1H), 7.79-7.76 (m, |
| | | | | 1H), 7.72-7.64 (m, 1H), 7.25-7.20 (m, 2H), 7.03- |
| | | | | 6.94 (m, 1H), 6.88 (dd, J = 4.8, 8.0 Hz, 1H), 6.54-6.46 |
| | | | | (m, 1H), 6.19-6.12 (m, 1H), 4.66-4.59 (m, 2H), 4.40- |
| | | | | 4.30 (m, 2H), 4.07-4.01 (m, 2H), 3.86 (br d, J = 8.4 |
| | | | | Hz, 1H), 3.82 (d, J = 3.2 Hz, 2H), 3.80 (br s, 3H), 3.66- |
| | | | | 3.58 (m, 2H), 3.38-3.36 (m, 3H), 3.09 (q, J = 6.8 Hz, |
| | | | | 2H), 2.54 (s, 2H), 2.36-2.33 (m, 1H), 2.28 (br s, 1H), |
| | | | | 1.35 (t, J = 6.8 Hz, 3H) |
| I-565 | DI | DF | 665.3 | 12.41 (br s, 1H), 8.90 (br d, J = 6.8 Hz, 1H), 8.86-8.81 |
| | | | | (m, 1H), 8.16-8.09 (m, 1H), 7.79 (d, J = 6.8 Hz, 1H), |
| | | | | 7.74-7.66 (m, 1H), 7.20-7.09 (m, 3H), 7.06-7.02 (m, |
| | | | | 1H), 6.96-6.91 (m, 1H), 6.70 (br s, 1H), 6.19 (br d, J = |
| | | | | 10.4 Hz, 1H), 4.66-4.61 (m, 2H), 4.38-4.31 (m, 2H), |
| | | | | 4.08 (s, 3H), 3.91 (br s, 4H), 3.82 (s, 3H), 3.67-3.59 |
| | | | | (m, 2H), 3.20-3.06 (m, 6H), 2.38-2.25 (m, 2H) |
| I-566 | DI | CY | 682.3 | 12.18 (br d, J = 1.6 Hz, 1H), 8.15-8.07 (m, 1H), 7.74- |
| | | | | 7.63 (m, 1H), 7.21 (br d, J = 8.4 Hz, 2H), 7.17-7.12 |
| | | | | (m, 1H), 7.00-6.92 (m, 3H), 6.91-6.85 (m, 2H), 6.46 |
| | | | | (br s, 1H), 6.13 (br s, 1H), 4.62 (br d, J = 6.8 Hz, 2H), |
| | | | | 4.39-4.30 (m, 2H), 3.81 (br s, 4H), 3.78 (br s, 3H), |
| | | | | 3.67-3.58 (m, 2H), 3.71 (br d, J = 1.6 Hz, 5H), 3.12- |
| | | | | 3.07 (m, 2H), 2.98 (br s, 3H), 2.37-2.26 (m, 2H) |
| I-567 | DI | DC | 682.3 | 12.34-12.21 (m, 1H), 8.15-8.07 (m, 1H), 7.73-7.63 |
| | | | | (m, 1H), 7.35-7.29 (m, 1H), 7.25-7.18 (m, 2H), 7.05- |
| | | | | 6.94 (m, 3H), 6.90-6.85 (m, 2H), 6.53 (br d, J = 2.8 |
| | | | | Hz, 1H), 6.21-6.12 (m, 1H), 4.62 (q, J = 7.2 Hz, 2H), |
| | | | | 4.40-4.32 (m, 2H), 3.80 (br d, J = 1.6 Hz, 4H), 3.78 (s, |
| | | | | 3H), 3.68-3.57 (m, 6H), 3.12-3.06 (m, 2H), 2.97 (br |
| | | | | s, 3H), 2.37-2.26 (m, 2H) |
| I-568 | FS | FQ | 650.3 | 12.31-12.21 (m, 1H), 8.51 (d, J = 5.6 Hz, 1H), 8.45- |
| | | | | 8.41 (m, 1H), 8.15-8.07 (m, 2H), 7.73-7.64 (m, 1H), |
| | | | | 7.57-7.51 (m, 1H), 7.21 (d, J = 5.6 Hz, 1H), 7.04- |
| | | | | 6.95 (m, 1H), 6.76 (d, J = 8.8 Hz, 1H), 6.63 (dd, J = 5.0, |
| | | | | 6.8 Hz, 1H), 6.53-6.46 (m, 1H), 6.19-6.12 (m, 1H), |
| | | | | 4.66-4.56 (m, 3H), 4.40-4.30 (m, 3H), 4.08-4.02 (m, |
| | | | | 1H), 3.88-3.79 (m, 4H), 3.65 (br t, J = 5.6 Hz, 1H), |
| | | | | 3.61-3.59 (m, 1H), 3.29 (br s, 2H), 3.14-3.07 (m, |
| | | | | 3H), 2.37-2.26 (m, 2H), 1.09-0.96 (m, 3H) |
| I-569 ^{f} | FT | DE | 653.4 | 12.13-11.94 (m, 1H), 8.14-8.07 (m, 1H), 7.84-7.77 |
| | | | | (m, 1H), 7.76-7.57 (m, 1H), 7.28 (d, J = 7.6 Hz, 1H), |
| | | | | 6.94 (s, 1H), 6.78-6.67 (m, 1H), 6.02-6.02 (m, 1H), |
| | | | | 6.52-5.82 (m, 1H), 4.62 (q, J = 6.4 Hz, 2H), 4.35- |
| | | | | 4.27 (m, 2H), 3.82 (s, 6H), 3.65-3.56 (m, 2H), 3.37- |
| | | | | 3.35 (m, 3H), 3.11-2.85 (m, 3H), 2.33 (s, 2H), 2.25 (s, |
| | | | | 2H), 2.10 (s, 3H), 1.71 (s, 3H), 1.43 (s, 3H), 0.80 (d, J = |
| | | | | 6.8 Hz, 1H) |
| I-570^{c, d} | FV | DE | 680.3 | 12.17 (br d, J = 7.2 Hz, 1H), 8.27 (s, 1H), 8.18-8.09 |
| | | | | (m, 1H), 7.79 (d, J = 4.4 Hz, 1H), 7.72-7.65 (m, 1H), |
| | | | | 7.27 (d, J = 8.0 Hz, 1H), 7.08 (s, 1H), 7.00-6.89 (m, |
| | | | | 2H), 6.47 (br s, 1H), 6.18-6.10 (m, 1H), 4.62 (q, J = |
| | | | | 7.2 Hz, 2H), 4.40-4.29 (m, 2H), 3.80 (s, 10H), 3.66- |
| | | | | 3.58 (m, 2H), 3.43-3.35 (m, 4H), 3.09 (q, J = 6.4 Hz, |
| | | | | 2H), 2.51 (br s, 3H), 2.37-2.25 (m, 2H) |
| I-571^{c, d} | FW | DE | 680.5 | 11.97 (d, J = 7.6 Hz, 1H), 8.15-8.07 (m, 1H), 7.83- |
| | | | | 7.77 (m, 1H), 7.72-7.64 (m, 1H), 7.27 (d, J = 7.2 Hz, |
| | | | | 1H), 6.99 (d, J = 8.0 Hz, 1H), 6.92 (dd, J = 4.8, 8.0 Hz, |
| | | | | 1H), 6.86-6.78 (m, 1H), 6.45 (s, 1H), 6.35 (s, 1H), |
| | | | | 6.25 (d, J = 7.6 Hz, 1H), 6.13-6.05 (m, 1H), 5.24 (s, |
| | | | | 2H), 4.65-4.59 (m, 2H), 4.37-4.28 (m, 2H), 3.80 (s, |
| | | | | 7H), 3.66-3.57 (m, 5H), 3.37-3.34 (m, 4H), 3.12- |
| | | | | 3.06 (m, 2H), 2.35-2.25 (m, 2H) |
| I-572^{c, d} | FY | DE | 653.3 | 12.12-12.00 (m, 1H), 8.32-8.22 (m, 1H), 8.14-8.08 |
| | | | | (m, 1H), 7.80 (d, J = 3.6 Hz, 1H), 7.73-7.65 (m, 1H), |
| | | | | 7.27 (dd, J = 1.2, 8.0 Hz, 1H), 7.00-6.85 (m, 3H), 6.11 |
| | | | | (br s, 1H), 4.62 (q, J = 7.2 Hz, 2H), 4.39-4.31 (m, 2H), |
| | | | | 3.81 (s, 8H), 3.65 (br d, J = 5.6 Hz, 4H), 3.12-3.08 (m, |
| | | | | 2H), 2.35 (br d, J = 3.2 Hz, 1H), 2.29 (s, 4H), 2.23 (s, 3H) |
| I-573 | BR | FZ | 660.3 | 12.43 (d, J = 4.4 Hz, 1H), 8.15-8.08 (m, 1H), 8.01 (d, |
| | | | | J = 7.6 Hz, 1H), 7.87-7.79 (m, 2H), 7.77-7.74 (m, |
| | | | | 1H), 7.72-7.67 (m, 1H), 7.63 (t, J = 7.6 Hz, 1H), 7.27 |
| | | | | (d, J = 8.0 Hz, 1H), 7.21-7.10 (m, 1H), 6.92 (dd, J = |
| | | | | 4.8, 7.6 Hz, 1H), 6.64 (s, 1H), 6.23-6.16 (m, 1H), 4.63 |
| | | | | (q, J = 7.2 Hz, 2H), 4.42-4.34 (m, 2H), 3.81 (s, 7H), |
| | | | | 3.67 (t, J = 5.2 Hz, 1H), 3.61 (t, J = 5.6 Hz, 1H), 3.29 |
| | | | | (s, 4H), 3.13-3.07 (m, 2H), 2.38-2.29 (m, 2H) |
| I-574 | BR | GA | 653.5 | 12.17 (s, 1H), 8.14-8.08 (m, 1H), 7.96-7.92 (m, 1H), |
| | | | | 7.80 (dd, J = 1.2, 4.8 Hz, 1H), 7.71-7.66 (m, 1H), 7.27 |
| | | | | (dd, J = 1.2, 8.0 Hz, 1H), 6.94-6.84 (m, 2H), 6.74 (s, |
| | | | | 1H), 6.16-6.07 (m, 1H), 4.62 (q, J = 7.2 Hz, 2H), 4.38- |
| | | | | 4.30 (m, 2H), 3.82 (d, J = 8.4 Hz, 10H), 3.66 (t, J = |
| | | | | 6.0 Hz, 1H), 3.60 (t, J = 5.6 Hz, 1H), 3.35 (s, 4H), 3.12- |
| | | | | 3.06 (m, 2H), 2.36 (s, 1H), 2.26 (s, 1H), 2.23 (s, 3H) |
| I-575^{c, d} | GB | DE | 661.4 | 12.44 (d, J = 6.4 Hz, 1H), 9.15 (d, J = 2.0 Hz, 1H), 8.40- |
| | | | | 8.36 (m, 1H), 8.25-8.21 (m, 1H), 8.13-8.09 (m, |
| | | | | 1H), 7.81 (dd, J = 1.2, 4.8 Hz, 1H), 7.71-7.68 (m, 1H), |
| | | | | 7.68-7.65 (m, 1H), 7.36 (dd, J = 2.8, 7.6 Hz, 1H), 7.28 |
| | | | | (dd, J = 1.2, 8.0 Hz, 1H), 6.93 (dd, J = 4.8, 8.0 Hz, 1H), |
| | | | | 6.21 (s, 1H), 4.66-4.64 (m, 1H), 4.63-4.60 (m, 1H), |
| | | | | 4.41-4.35 (m, 2H), 3.85-3.83 (m, 2H), 3.81 (s, 3H), |
| | | | | 3.67 (t, J = 5.6 Hz, 1H), 3.62 (t, J = 5.6 Hz, 1H), 3.37 |
| | | | | (s, 6H), 3.14-3.09 (m, 2H), 2.39-2.29 (m, 2H) |
| I-576 | BR | GC | 690.2 | 12.22 (d, J = 6.4 Hz, 1H), 8.14-8.07 (m, 1H), 7.79 (dd, |
| | | | | J = 1.2, 4.8 Hz, 1H), 7.71-7.61 (m, 2H), 7.57-7.50 |
| | | | | (m, 2H), 7.27 (dd, J = 1.2, 8.0 Hz, 1H), 7.04-6.94 (m, |
| | | | | 1H), 6.92 (dd, J = 4.8, 8.0 Hz, 1H), 6.48 (s, 1H), 6.17- |
| | | | | 6.11 (m, 1H), 4.65-4.59 (m, 2H), 4.38-4.29 (m, 2H), |
| | | | | 3.82-3.77 (m, 10H), 3.65 (t, J = 5.6 Hz, 1H), 3.59 (t, J = |
| | | | | 5.6 Hz, 1H), 3.35-3.32 (m, 4H), 3.12-3.06 (m, 2H), |
| | | | | 2.37-2.26 (m, 2H) |
| I-577^{c, d} | GD | DE | 716.5 | 12.40-12.21 (m, 1H), 9.03-8.84 (m, 1H), 8.59 (d, J = |
| | | | | 8.4 Hz, 1H), 8.13-8.05 (m, 1H), 7.93-7.87 (m, 1H), |
| | | | | 7.86-7.81 (m, 1H), 7.80-7.73 (m, 1H), 7.72-7.58 (m, |
| | | | | 2H), 7.30-7.09 (m, 2H), 6.90 (dd, J = 4.8, 7.6 Hz, 1H), |
| | | | | 6.59 (s, 1H), 6.23-6.12 (m, 1H), 4.66-4.57 (m, 2H), |
| | | | | 4.45-4.32 (m, 2H), 3.78 (s, 7H), 3.70-3.55 (m, 3H), |
| | | | | 3.52-3.43 (m, 3H), 3.15-3.04 (m, 2H), 2.39-2.24 (m, 2H) |
| I-578 | GE | B | 674.4 | 12.53 (br d, J = 3.6 Hz, 1H), 8.15-8.05 (m, 1H), 7.78 |
| | | | | (d, J = 4.8 Hz, 1H), 7.74-7.66 (m, 3H), 7.38-7.33 (m, |
| | | | | 1H), 7.25 (d, J = 8.0 Hz, 1H), 7.18 (br d, J = 7.2 Hz, |
| | | | | 1H), 7.15-7.10 (m, 2H), 6.91 (dd, J = 4.8, 8.0 Hz, 1H), |
| | | | | 6.74 (d, J = 3.2 Hz, 1H), 6.49 (br s, 1H), 6.26-6.17 (m, |
| | | | | 1H), 4.65-4.59 (m, 2H), 4.43-4.34 (m, 2H), 3.79 (s, |
| | | | | 3H), 3.78-3.74 (m, 4H), 3.59 (br s, 2H), 3.30-3.25 |
| | | | | (m, 4H), 3.10 (q, J = 6.4 Hz, 2H), 2.37-2.27 (m, 2H) |
| I-579 | FB | B | 641.4 | 12.04-11.97 (m, 1H), 8.14-8.09 (m, 1H), 7.82 (dd, J = |
| | | | | 1.2, 4.8 Hz, 1H), 7.72-7.67 (m, 1H), 7.29 (dd, J = |
| | | | | 1.2, 8.0 Hz, 1H), 6.96-6.90 (m, 2H), 6.82-6.73 (m, |
| | | | | 1H), 6.09-6.03 (m, 1H), 4.63 (q, J = 6.4 Hz, 2H), 4.35- |
| | | | | 4.27 (m, 2H), 3.83 (s, 7H), 3.67-3.58 (m, 2H), 3.38 |
| | | | | (br s, 4H), 3.13-3.06 (m, 2H), 2.96-2.89 (m, 1H), |
| | | | | 2.36-2.26 (m, 2H), 1.90-1.79 (m, 4H), 1.73 (br s, |
| | | | | 1H), 1.58-1.44 (m, 4H), 1.34-1.25 (m, 1H) |
| I-580^{c, d} | GG | DE | 694.3 | 12.16 (d, J = 5.2 Hz, 1H), 8.15-8.09 (m, 1H), 7.78 (d, |
| | | | | J = 5.6 Hz, 1H), 7.72-7.63 (m, 1H), 7.54 (d, J = 7.6 |
| | | | | Hz, 1H), 7.38 (d, J = 8.0 Hz, 1H), 7.08 (dd, J = 5.6, 7.6 |
| | | | | Hz, 2H), 6.97-6.87 (m, 2H), 6.48 (s, 1H), 6.16-6.10 |
| | | | | (m, 1H), 4.68-4.58 (m, 4H), 4.37-4.30 (m, 2H), 3.90- |
| | | | | 3.83 (m, 7H), 3.75 (d, J = 3.4 Hz, 3H), 3.65 (t, J = 5.2 |
| | | | | Hz, 1H), 3.59 (d, J = 4.0 Hz, 4H), 3.09 (q, J = 7.6 Hz, |
| | | | | 2H), 2.92 (s, 3H), 2.37-2.26 (m, 2H) |
| I-581^{c, d} | GK | DE | 661.3 | 12.42-12.32 (m, 1H), 8.96 (d, J = 4.8 Hz, 1H), 8.48 (s, |
| | | | | 1H), 8.14-8.09 (m, 1H), 7.80 (br d, J = 3.6 Hz, 2H), |
| | | | | 7.71-7.62 (m, 2H), 7.39 (br s, 1H), 7.27 (dd, J = 1.2, |
| | | | | 8.0 Hz, 1H), 6.92 (dd, J = 4.8, 8.0 Hz, 1H), 6.25-6.20 |
| | | | | (m, 1H), 4.66-4.61 (m, 2H), 4.42-4.36 (m, 2H), 3.81 |
| | | | | (s, 8H), 3.68 (br t, J = 4.8 Hz, 1H), 3.62 (br s, 1H), 3.38- |
| | | | | 3.35 (m, 3H), 3.13-3.07 (m, 2H), 2.38 (br d, J = 1.6 |
| | | | | Hz, 1H), 2.34-2.28 (m, 1H) |
| I-582^{c, d} | GL | DE | 653.4 | 12.41 (br s, 1H), 8.15-8.08 (m, 1H), 7.82-7.76 (m, |
| | | | | 1H), 7.73-7.64 (m, 1H), 7.58 (d, J = 1.6 Hz, 1H), 7.27 |
| | | | | (d, J = 8.0 Hz, 1H), 7.07-6.97 (m, 1H), 6.92 (dd, J = |
| | | | | 4.8, 7.8 Hz, 1H), 6.57 (d, J = 2.8 Hz, 1H), 6.44 (s, 1H), |
| | | | | 6.18 (br d, J = 13.2 Hz, 1H), 4.62 (q, J = 6.8 Hz, 2H), |
| | | | | 4.39-4.32 (m, 2H), 4.07 (q, J = 7.2 Hz, 2H), 3.80 (s, |
| | | | | 8H), 3.67-3.59 (m, 2H), 3.36-3.34 (m, 3H), 3.10 (q, J = |
| | | | | 6.4 Hz, 2H), 2.37-2.27 (m, 2H), 1.26 (t, J = 7.2 Hz, 3H) |
| I-583^{c, d} | GO | DE | 723.2 | 12.11-12.07 (m, 1H), 8.14-8.10 (m, 1H), 7.80 (br d, J = |
| | | | | 4.8 Hz, 1H), 7.71-7.67 (m, 1H), 7.27 (br d, J = 8.0 |
| | | | | Hz, 1H), 6.94-6.85 (m, 3H), 6.68 (d, J = 8.4 Hz, 1H), |
| | | | | 6.56 (br s, 1H), 6.15-6.09 (m, 1H), 4.66-4.60 (m, |
| | | | | 2H), 4.38-4.31 (m, 2H), 4.27-4.18 (m, 4H), 3.81 (br |
| | | | | s, 12H), 3.67-3.60 (m, 2H), 3.36-3.35 (m, 2H), 3.13- |
| | | | | 3.08 (m, 2H), 2.37-2.34 (m, 1H), 2.31-2.24 (m, 1H) |
| I-584 | EW | D | 612.0 | |
| I-585 | GS | B | 642.2 | 12.08 (br s, 1H), 8.24 (s, 1H), 8.14-8.09 (m, 1H), 7.81 |
| | | | | (br d, J = 4.8 Hz, 1H), 7.72-7.66 (m, 1H), 7.28 (br d, J = |
| | | | | 8.0 Hz, 1H), 7.03 (br s, 1H), 6.95-6.85 (m, 2H), 6.06 |
| | | | | (br s, 1H), 4.65-4.60 (m, 2H), 4.36-4.24 (m, 3H), |
| | | | | 3.78-3.71 (m, 2H), 3.67-3.63 (m, 1H), 3.59 (br s, |
| | | | | 1H), 3.38 (br s, 4H), 3.12-3.06 (m, 2H), 3.01 (br t, J = |
| | | | | 8.4 Hz, 1H), 2.87 (br d, J = 4.4 Hz, 1H), 2.81-2.73 (m, |
| | | | | 2H), 2.54 (s, 1H), 2.52-2.50 (m, 4H), 2.43 (s, 3H), |
| | | | | 2.34 (br s, 2H), 2.27 (br s, 1H), 2.00-1.92 (m, 1H) |
| I-586 | GT | B | 653.3 | CHLOROFORM-d) δ = 9.63-9.50 (m, 1H), 7.89 (d, J = |
| | | | | 4.8 Hz, 1H), 7.76 (d, J = 2.8 Hz, 1H), 7.67 (d, J = 3.2 |
| | | | | Hz, 1H), 7.10 (d, J = 8.0 Hz, 1H), 6.97 (t, J = 4.8 Hz, |
| | | | | 1H), 6.91 (dd, J = 5.2, 7.8 Hz, 1H), 6.66 (s, 1H), 6.16 |
| | | | | (d, J = 17.2 Hz, 1H), 6.06 (d, J = 4.0 Hz, 1H), 4.82- |
| | | | | 4.78 (m, 2H), 4.46 (s, 1H), 4.28 (s, 1H), 4.03 (s, 4H), |
| | | | | 3.89 (s, 3H), 3.77 (t, J = 5.6 Hz, 1H), 3.59-3.56 (m, |
| | | | | 1H), 3.51 (s, 4H), 3.06 (q, J = 6.0 Hz, 2H), 2.38-2.36 |
| | | | | (m, 1H), 2.34 (s, 4H), 2.22 (d, J = 4.8 Hz, 3H) |
| I-587 | GZ | DF | 599.3 | 12.18 (d, J = 3.2 Hz, 1H), 8.50 (d, J = 5.6 Hz, 1H), 8.40 |
| | | | | (d, J = 1.6 Hz, 1H), 8.10 (d, J = 11.2 Hz, 1H), 7.68 (d, J = |
| | | | | 12.0 Hz, 1H), 7.21 (d, J = 5.6 Hz, 1H), 7.02-6.93 |
| | | | | (m, 1H), 6.40 (s, 1H), 6.17-6.12 (m, 1H), 4.65-4.59 |
| | | | | (m, 2H), 4.38-4.30 (m, 2H), 3.81 (d, J = 3.2 Hz, 3H), |
| | | | | 3.64-3.58 (m, 6H), 3.09 (q, J = 6.4 Hz, 2H), 2.55 (r s, |
| | | | | 4H), 2.35-2.26 (m, 2H), 1.64 (tt, J = 3.2, 6.4 Hz, 1H), |
| | | | | 0.45-0.41 (m, 2H), 0.33 (d, J = 2.8 Hz, 2H) |
| I-588 | GZ | HA | 616.4 | 12.12 (d, J = 7.2 Hz, 1H), 8.14-8.07 (m, 1H), 7.73- |
| | | | | 7.58 (m, 1H), 7.35 (t, J = 7.6 Hz, 1H), 7.05 (d, J = 11.2 |
| | | | | Hz, 1H), 6.95-6.84 (m, 2H), 6.37 (s, 1H), 6.12 (d, J = |
| | | | | 11.2 Hz, 1H), 4.62 (q, J = 7.2 Hz, 2H), 4.37-4.29 (m, |
| | | | | 2H), 3.74 (d, J = 2.4 Hz, 3H), 3.66-3.58 (m, 6H), 3.09 |
| | | | | (q, J = 7.2 Hz, 2H), 2.54 (s, 4H), 2.36-2.25 (m, 2H), |
| | | | | 1.82-1.47 (m, 1H), 0.59-0.18 (m, 4H) |
| I-589 | HC | HA | 683.3 | 12.16 (s, 1H), 8.14-8.07 (m, 1H), 7.72-7.66 (m, 1H), |
| | | | | 7.48 (t, J = 8.0 Hz, 1H), 7.37 (d, J = 7.6 Hz, 1H), 7.06 |
| | | | | (d, J = 11.2 Hz, 1H), 6.97-6.86 (m, 2H), 6.47 (s, 1H), |
| | | | | 6.34 (d, J = 8.0 Hz, 1H), 6.13 (s, 1H), 6.08 (d, J = 8.0 |
| | | | | Hz, 1H), 4.66-4.61 (m, 2H), 4.39-4.31 (m, 2H), 3.80- |
| | | | | 3.75 (m, 10H), 3.67-3.60 (m, 2H), 3.57 (s, 4H), 3.13- |
| | | | | 3.07 (m, 2H), 2.35 (d, J = 1.2 Hz, 2H) |
| I-590 | HC | FD | 701.3 | 12.06 (d, J = 5.6 Hz, 1H), 8.02-7.93 (m, 1H), 7.60- |
| | | | | 7.52 (m, 1H), 7.37-7.30 (m, 2H), 7.18 (dd, J = 7.2, |
| | | | | 13.2 Hz, 1H), 6.88-6.78 (m, 1H), 6.38 (s, 1H), 6.21 (d, |
| | | | | J = 8.4 Hz, 1H), 6.07-5.99 (m, 1H), 5.95 (d, J = 8.0 |
| | | | | Hz, 1H), 4.54-4.48 (m, 2H), 4.26-4.18 (m, 2H), 3.65 |
| | | | | (s, 7H), 3.62 (s, 3H), 3.54-3.48 (m, 2H), 3.46 (d, J = |
| | | | | 9.6 Hz, 4H), 3.01-2.94 (m, 2H), 2.23-2.14 (m, 2H) |
| I-591 | HC | HB | 701.3 | 12.32 (s, 1H), 8.18-8.05 (m, 1H), 7.75-7.62 (m, 1H), |
| | | | | 7.47 (t, J = 8.0 Hz, 1H), 7.44-7.37 (m, 1H), 7.18-7.12 |
| | | | | (m, 1H), 7.02 (d, J = 6.4 Hz, 1H), 6.61 (s, 1H), 6.33 (d, |
| | | | | J = 8.0 Hz, 1H), 6.22-6.14 (m, 1H), 6.07 (d, J = 8.0 |
| | | | | Hz, 1H), 4.67-4.61 (m, 2H), 4.41-4.32 (m, 2H), 3.78 |
| | | | | (s, 6H), 3.68-3.61 (m, 2H), 3.56 (d, J = 3.2 Hz, 8H), |
| | | | | 3.14-3.08 (m, 2H), 2.36 (d, J = 3.2 Hz, 2H) |
| I-592 | HE | B | 658.4 | 11.95 (s, 1H), 8.14-8.08 (m, 1H), 7.81 (dd, J = 1.2, 4.8 |
| | | | | Hz, 1H), 7.75-7.64 (m, 1H), 7.28 (dd, J = 1.2, 8.0 Hz, |
| | | | | 1H), 6.93 (dd, J = 4.8, 8.0 Hz, 1H), 6.72 (s, 1H), 6.23- |
| | | | | 6.16 (m, 1H), 6.05 (s, 1H), 4.65-4.59 (m, 2H), 4.33- |
| | | | | 4.25 (m, 2H), 3.82 (s, 9H), 3.77-3.74 (m, 2H), 3.64 (t, |
| | | | | J = 5.6 Hz, 1H), 3.58 (t, J = 5.6 Hz, 1H), 3.52 (s, 4H), |
| | | | | 3.37 (s, 4H), 3.11-3.04 (m, 2H), 2.34-2.24 (m, 2H), |
| | | | | 2.00 (quin, J = 5.6 Hz, 2H) |
| I-593 | HI | CG | 692.5 | 12.24 (br d, J = 6.1 Hz, 1H), 8.50 (s, 1H), 8.32-8.28 |
| | | | | (m, 1H), 8.14-8.08 (m, 1H), 7.81 (dd, J = 1.4, 4.8 Hz, |
| | | | | 1H), 7.72-7.66 (m, 1H), 7.53 (dd, J = 1.3, 7.9 Hz, 1H), |
| | | | | 7.42-7.37 (m, 1H), 7.09-6.99 (m, 1H), 6.93 (dd, J = |
| | | | | 4.8, 7.9 Hz, 1H), 6.52 (br s, 1H), 6.18-6.11 (m, 1H), |
| | | | | 4.62 (q, J = 7.5 Hz, 2H), 4.38-4.30 (m, 2H), 3.91- |
| | | | | 3.82 (m, 5H), 3.77 (br d, J = 1.3 Hz, 4H), 3.66-3.58 |
| | | | | (m, 2H), 3.29-3.27 (m, 3H), 3.12-3.06 (m, 2H), 2.36- |
| | | | | 2.33 (m, 1H), 2.28 (br s, 1H), 0.83-0.76 (m, 2H), |
| | | | | 0.70 (br s, 2H) |
| I-594 | HI | HA | 709.5 | 12.13 (br d, J = 6.4 Hz, 1H), 8.14-8.07 (m, 1H), 7.81 |
| | | | | (dd, J = 1.3, 4.8 Hz, 1H), 7.71-7.65 (m, 1H), 7.53 (dd, |
| | | | | J = 1.3, 8.0 Hz, 1H), 7.37 (t, J = 7.6 Hz, 1H), 7.05 (br d, |
| | | | | J = 11.3 Hz, 1H), 6.95-6.84 (m, 3H), 6.43 (br s, 1H), |
| | | | | 6.15-6.09 (m, 1H), 4.62 (q, J = 7.0 Hz, 2H), 4.38- |
| | | | | 4.30 (m, 2H), 3.88 (tt, J = 2.9, 5.9 Hz, 1H), 3.81-3.70 |
| | | | | (m, 8H), 3.66-3.58 (m, 2H), 3.29-3.27 (m, 3H), 3.09 |
| | | | | (q, J = 7.1 Hz, 2H), 2.34 (br d, J = 3.3 Hz, 1H), 2.27 (br |
| | | | | s, 1H), 0.82-0.76 (m, 2H), 0.70 (br s, 2H) |
| I-595 | HI | DQ | 692.3 | 12.20 (br d, J = 6.8 Hz, 1H), 8.23 (br d, J = 5.0 Hz, |
| | | | | 1H), 8.13-8.07 (m, 1H), 7.83-7.78 (m, 2H), 7.71- |
| | | | | 7.65 (m, 1H), 7.53 (dd, J = 1.4, 7.9 Hz, 1H), 7.12 (dd, J = |
| | | | | 5.1, 7.1 Hz, 1H), 7.05-6.97 (m, 1H), 6.93 (dd, J = |
| | | | | 4.9, 7.9 Hz, 1H), 6.50 (br s, 1H), 6.17-6.11 (m, 1H), |
| | | | | 4.65-4.59 (m, 2H), 4.38-4.30 (m, 2H), 3.91-3.81 (m, |
| | | | | 5H), 3.77 (br s, 4H), 3.66-3.58 (m, 2H), 3.29-3.28 |
| | | | | (m, 3H), 3.09 (br d, J = 7.3 Hz, 2H), 2.34 (br s, 1H), |
| | | | | 2.28 (br d, J = 2.4 Hz, 1H), 0.80 (br d, J = 5.6 Hz, 2H), |
| | | | | 0.70 (br s, 2H) |
| I-596 | HI | FD | 727.4 | 12.17 (br d, J = 5.6 Hz, 1H), 8.13-8.08 (m, 1H), 7.83- |
| | | | | 7.79 (m, 1H), 7.71-7.66 (m, 1H), 7.53 (dd, J = 1.0, 7.9 |
| | | | | Hz, 1H), 7.47-7.41 (m, 1H), 7.30 (br dd, J = 6.9, 13.1 |
| | | | | Hz, 1H), 6.99-6.90 (m, 2H), 6.47 (br s, 1H), 6.17- |
| | | | | 6.11 (m, 1H), 4.62 (q, J = 7.0 Hz, 2H), 4.38-4.29 (m, |
| | | | | 2H), 3.91-3.86 (m, 1H), 3.77 (br s, 4H), 3.73 (br d, J = |
| | | | | 2.3 Hz, 4H), 3.65 (br t, J = 5.5 Hz, 1H), 3.61-3.58 (m, |
| | | | | 1H), 3.30-3.28 (m, 3H), 3.09 (br d, J = 7.4 Hz, 2H), |
| | | | | 2.34 (br d, J = 3.0 Hz, 1H), 2.27 (br d, J = 3.1 Hz, 1H), |
| | | | | 0.82-0.77 (m, 2H), 0.70 (br s, 2H) |
| I-597 | HI | HB | 727.3 | 12.31-12.23 (m, 1H), 8.13-8.08 (m, 1H), 7.81 (dd, J = |
| | | | | 1.3, 4.8 Hz, 1H), 7.71-7.65 (m, 1H), 7.53 (dd, J = |
| | | | | 1.4, 7.9 Hz, 1H), 7.42-7.36 (m, 1H), 7.14 (dt, J = 2.0, |
| | | | | 5.5 Hz, 1H), 7.08-7.00 (m, 1H), 6.93 (dd, J = 4.9, 7.9 |
| | | | | Hz, 1H), 6.57 (br s, 1H), 6.19-6.14 (m, 1H), 4.62 (q, J = |
| | | | | 6.8 Hz, 2H), 4.39-4.31 (m, 2H), 3.88 (tt, J = 2.9, 5.8 |
| | | | | Hz, 1H), 3.76 (br s, 4H), 3.66-3.59 (m, 2H), 3.56- |
| | | | | 3.48 (m, 4H), 3.28-3.24 (m, 3H), 3.09 (br d, J = 6.9 |
| | | | | Hz, 2H), 2.35 (br d, J = 1.5 Hz, 1H), 2.27 (br d, J = 1.5 |
| | | | | Hz, 1H), 0.82-0.77 (m, 2H), 0.72-0.68 (m, 2H) |
| I-598 | DI | HA | 682.3 | 12.22-12.10 (m, 1H), 8.21-8.04 (m, 1H), 7.75-7.62 |
| | | | | (m, 1H), 7.42-7.33 (m, 1H), 7.05-7.03 (m, 1H), 7.02- |
| | | | | 6.96 (m, 2H), 6.97-6.85 (m, 4H), 6.44 (br s, 1H), |
| | | | | 6.15-6.10 (m, 1H), 4.66-4.60 (m, 2H), 4.40-4.29 |
| | | | | (m, 2H), 3.92-3.77 (m, 6H), 3.76-3.73 (m, 3H), 3.66- |
| | | | | 3.60 (m, 2H), 3.11-3.06 (m, 2H), 2.99 (br s, 4H), |
| | | | | 2.02-2.00 (m, 1H) |
| I-599 | DI | DQ | 665.2 | 12.32-12.11 (m, 1H), 8.28-8.20 (m, 1H), 8.16-8.07 |
| | | | | (m, 1H), 7.91-7.75 (m, 1H), 7.75-7.63 (m, 1H), 7.19- |
| | | | | 7.09 (m, 1H), 7.06-6.83 (m, 5H), 6.51 (br s, 1H), |
| | | | | 6.19-6.10 (m, 1H), 4.63 (q, J = 7.2 Hz, 2H), 4.43- |
| | | | | 4.29 (m, 2H), 3.90-3.77 (m, 10H), 3.69-3.59 (m, 2H), |
| | | | | 3.16-3.05 (m, 3H), 2.99 (br s, 3H), 2.35-2.29 (m, 2H) |
| I-600 | DI | FD | 700.2 | 12.42-12.21 (m, 1H), 8.26 (s, 1H), 7.88-7.74 (m, |
| | | | | 1H), 7.61-7.49 (m, 1H), 7.46-7.35 (m, 1H), 7.10- |
| | | | | 6.96 (m, 5H), 6.58 (br s, 1H), 6.33-6.19 (m, 1H), 4.77- |
| | | | | 4.69 (m, 2H), 4.48-4.40 (m, 2H), 3.95-3.87 (m, |
| | | | | 7H), 3.83 (d, J = 2.4 Hz, 3H), 3.77-3.60 (m, 3H), 3.25- |
| | | | | 3.15 (m, 2H), 3.08 (br s, 3H), 2.47-2.36 (m, 2H) |
| I-601 | DI | HB | 700.1 | 12.35-12.21 (m, 1H), 8.18-8.09 (m, 1H), 7.81-7.65 |
| | | | | (m, 1H), 7.43-7.38 (m, 1H), 7.18-7.09 (m, 1H), 7.05- |
| | | | | 6.93 (m, 3H), 6.90-6.82 (m, 2H), 6.58 (s, 1H), 6.16- |
| | | | | 6.08 (m, 1H), 4.68-4.63 (m, 2H), 4.62-4.33 (m, 2H), |
| | | | | 3.83-3.75 (m, 7H), 3.71-3.40 (m, 5H), 3.15-3.11 |
| | | | | (m, 2H), 3.10-3.08 (m, 4H), 2.37-2.35 (m, 2H) |
| I-602 | GZ | DQ | 599.6 | 12.18 (s, 1H), 8.23 (d, J = 4.4 Hz, 1H), 8.15-8.06 (m, |
| | | | | 1H), 7.79 (r d, J = 7.2 Hz, 1H), 7.74-7.62 (m, 1H), |
| | | | | 7.11 (dd, J = 5.6, 6.4 Hz, 1H), 7.05-6.96 (m, 1H), 6.43 |
| | | | | (s, 1H), 6.18-6.11 (m, 1H), 4.62 (q, J = 7.6 Hz, 2H), |
| | | | | 4.38-4.29 (m, 2H), 3.83 (d, J = 4.0 Hz, 3H), 3.66- |
| | | | | 3.58 (m, 6H), 3.12-3.06 (m, 2H), 2.55 (s, 4H), 2.36- |
| | | | | 2.26 (m, 2H), 1.67-1.61 (m, 1H), 0.45-0.31 (m, 4H) |
| I-603 | GZ | HB | 634.4 | 12.27 (d, J = 5.2 Hz, 1H), 8.14-8.06 (m, 1H), 7.89- |
| | | | | 7.53 (m, 1H), 7.39 (ddd, J = 3.2, 8.4, 11.6 Hz, 1H), 7.13 |
| | | | | (dd, J = 3.6, 5.2 Hz, 1H), 7.07-6.99 (m, 1H), 6.50 (s, |
| | | | | 1H), 6.20-6.13 (m, 1H), 4.62 (q, J = 6.8 Hz, 2H), 4.39- |
| | | | | 4.30 (m, 2H), 3.67-3.58 (m, 6H), 3.53 (d, J = 2.4 Hz, |
| | | | | 3H), 3.09 (q, J = 7.2 Hz, 2H), 2.55 (s, 4H), 2.37-2.26 |
| | | | | (m, 2H), 1.68-1.61 (m, 1H), 0.45-0.30 (m, 4H) |
| I-604 | GZ | FD | 634.4 | 12.16 (d, J = 5.2 Hz, 1H), 8.15-8.07 (m, 1H), 7.82- |
| | | | | 7.53 (m, 1H), 7.47-7.40 (m, 1H), 7.29 (dd, J = 6.8, |
| | | | | 12.8 Hz, 1H), 6.98-6.89 (m, 1H), 6.40 (s, 1H), 6.16- |
| | | | | 6.10 (m, 1H), 4.62 (q, J = 6.8 Hz, 2H), 4.37-4.29 (m, |
| | | | | 2H), 3.72 (s, 3H), 3.65-3.58 (m, 6H), 3.12-3.06 (m, |
| | | | | 2H), 2.55 (s, 4H), 2.35-2.25 (m, 2H), 1.68-1.62 (m, |
| | | | | 1H), 0.45-0.31 (m, 4H) |
| I-605 | GZ | CG | 599.2 | 12.24 (d, J = 6.4 Hz, 1H), 8.50 (s, 1H), 8.30 (d, J = 4.4 |
| | | | | Hz, 1H), 8.13-8.07 (m, 1H), 7.80-7.63 (m, 1H), 7.40 |
| | | | | (d, J = 4.4 Hz, 1H), 7.08-6.98 (m, 1H), 6.45 (s, 1H), |
| | | | | 6.18-6.11 (m, 1H), 4.62 (q, J = 7.2 Hz, 2H), 4.38- |
| | | | | 4.29 (m, 2H), 3.85 (d, J = 3.2 Hz, 3H), 3.67-3.58 (m, |
| | | | | 6H), 3.12-3.06 (m, 2H), 2.55 (s, 4H), 2.36-2.25 (m, |
| | | | | 2H), 1.64 (dd, J = 3.2, 6.4 Hz, 1H), 0.47-0.28 (m, 4H) |
| I-606 | HC | DQ | 666.3 | 12.23 (d, J = 6.0 Hz, 1H), 8.24 (dd, J = 1.6, 4.8 Hz, |
| | | | | 1H), 8.15-8.10 (m, 1H), 7.81 (td, J = 2.4, 7.2 Hz, 1H), |
| | | | | 7.72-7.64 (m, 1H), 7.47 (t, J = 8.0 Hz, 1H), 7.12 (dd, J = |
| | | | | 4.8, 7.2 Hz, 1H), 7.07-6.98 (m, 1H), 6.53 (s, 1H), |
| | | | | 6.33 (d, J = 8.0 Hz, 1H), 6.19-6.12 (m, 1H), 6.07 (d, J = |
| | | | | 8.0 Hz, 1H), 4.67-4.60 (m, 2H), 4.40-4.31 (m, 2H), |
| | | | | 3.87-3.84 (m, 3H), 3.78 (s, 7H), 3.66 (t, J = 6.0 Hz, |
| | | | | 1H), 3.63-3.59 (m, 2H), 3.57 (s, 3H), 3.13-3.08 (m, |
| | | | | 2H), 2.38-2.28 (m, 2H) |
| I-607^{d} | HO | DE | 678.3 | 12.45-12.19 (m, 1H), 8.60-8.47 (m, 1H), 8.39 (s, |
| | | | | 1H), 8.15-8.05 (m, 1H), 7.83-7.61 (m, 2H), 7.49 (t, J = |
| | | | | 5.2 Hz, 1H), 7.32-7.18 (m, 1H), 6.97-6.80 (m, 2H), |
| | | | | 6.37-6.10 (m, 2H), 4.62 (d, J = 6.4 Hz, 2H), 4.41- |
| | | | | 4.31 (m, 2H), 3.81-3.73 (m, 6H), 3.64-3.58 (m, 2H), |
| | | | | 3.10 (d, J = 6.4 Hz, 6H), 2.89-2.81 (m, 2H), 2.35- |
| | | | | 2.27 (m, 4H), 1.13-1.04 (m, 4H) |
| I-608 | HP | DE | 664.3 | 12.36 (d, J = 4.8 Hz, 1H), 8.66-8.55 (m, 1H), 8.52- |
| | | | | 8.44 (m, 1H), 8.17-8.05 (m, 1H), 7.82-7.74 (m, 1H), |
| | | | | 7.74-7.62 (m, 1H), 7.31-7.22 (m, 2H), 7.00-6.87 (m, |
| | | | | 2H), 6.42-6.35 (m, 1H), 6.22-6.13 (m, 1H), 4.66- |
| | | | | 4.59 (m, 2H), 4.41-4.30 (m, 2H), 3.80 (s, 3H), 3.77 (s, |
| | | | | 4H), 3.67-3.58 (m, 2H), 3.10 (q, J = 6.8 Hz, 2H), 2.70- |
| | | | | 2.52 (m, 3H), 2.50-2.45 (m, 3H), 2.37-2.24 (m, |
| | | | | 2H), 0.98 (t, J = 7.6 Hz, 3H) |
| I-609 | HI | HW | 734.4 | 12.04-11.94 (m, 1H), 8.15-8.07 (m, 1H), 7.81 (dd, J = |
| | | | | 1.2, 4.8 Hz, 1H), 7.74-7.62 (m, 1H), 7.53 (dd, J = |
| | | | | 1.2, 7.6 Hz, 1H), 7.15 (d, J = 8.4 Hz, 1H), 6.93 (dd, J = |
| | | | | 4.8, 8.0 Hz, 1H), 6.89-6.81 (m, 1H), 6.43 (d, J = 8.4 |
| | | | | Hz, 2H), 6.41-6.38 (m, 1H), 6.14-6.07 (m, 1H), 4.65- |
| | | | | 4.59 (m, 2H), 4.37-4.29 (m, 2H), 3.88 (dt, J = 3.2, |
| | | | | 5.6 Hz, 1H), 3.78 (s, 4H), 3.73-3.70 (m, 3H), 3.65 (t, J = |
| | | | | 6.4 Hz, 1H), 3.59 (t, J = 5.6 Hz, 1H), 3.29-3.27 (m, |
| | | | | 4H), 3.12-3.07 (m, 2H), 2.97 (s, 6H), 2.34-2.27 (m, |
| | | | | 2H), 0.81-0.69 (m, 4H) |
| I-610 | DI | HW | 707.4 | 12.08-11.96 (m, 1H), 8.18-8.05 (m, 1H), 7.87-7.48 |
| | | | | (m, 1H), 7.15 (d, J = 8.4 Hz, 1H), 7.00-6.93 (m, 2H), |
| | | | | 6.91-6.87 (m, 2H), 6.86-6.81 (m, 1H), 6.46-6.37 (m, |
| | | | | 3H), 6.14-6.08 (m, 1H), 4.65-4.58 (m, 2H), 4.37- |
| | | | | 4.29 (m, 2H), 3.82 (s, 4H), 3.78 (s, 3H), 3.73-3.70 (m, |
| | | | | 3H), 3.65 (t, J = 5.6 Hz, 1H), 3.59 (t, J = 5.6 Hz, 1H), |
| | | | | 3.09 (q, J = 7.2 Hz, 2H), 2.97 (s, 10H), 2.35-2.25 (m, 2H) |
| I-611 | GZ | HW | 641.4 | 11.99 (d, J = 6.4 Hz, 1H), 8.14-8.07 (m, 1H), 7.75- |
| | | | | 7.60 (m, 1H), 7.14 (d, J = 8.4 Hz, 1H), 6.89-6.79 (m, |
| | | | | 1H), 6.43-6.36 (m, 3H), 6.13-6.06 (m, 1H), 4.65- |
| | | | | 4.59 (m, 2H), 4.37-4.28 (m, 2H), 3.71 (d, J = 3.6 Hz, |
| | | | | 3H), 3.65-3.57 (m, 6H), 3.09 (q, J = 7.2 Hz, 2H), 2.97 |
| | | | | (s, 6H), 2.55 (s, 4H), 2.35-2.25 (m, 2H), 1.64 (dt, J = |
| | | | | 3.2, 6.4 Hz, 1H), 0.45-0.32 (m, 4H) |
| I-612 | HC | CG | 666.3 | 12.27 (d, J = 2.0 Hz, 1H), 8.52 (s, 1H), 8.31 (d, J = 4.4 |
| | | | | Hz, 1H), 8.16-8.09 (m, 1H), 7.73-7.65 (m, 1H), 7.48 |
| | | | | (t, J = 8.0 Hz, 1H), 7.42 (d, J = 4.8 Hz, 1H), 7.13-7.01 |
| | | | | (m, 1H), 6.56 (s, 1H), 6.34 (d, J = 8.0 Hz, 1H), 6.19- |
| | | | | 6.13 (m, 1H), 6.08 (d, J = 8.0 Hz, 1H), 4.63 (q, J = 7.2 |
| | | | | Hz, 2H), 4.39-4.31 (m, 2H), 3.88 (d, J = 3.2 Hz, 3H), |
| | | | | 3.78 (s, 7H), 3.69-3.61 (m, 2H), 3.58 (s, 4H), 3.16- |
| | | | | 3.08 (m, 2H), 2.39-2.29 (m, 2H) |
| I-613 | IC | HA | 701.3 | 12.12 (d, J = 6.0 Hz, 1H), 8.14-8.07 (m, 1H), 7.79 (d, |
| | | | | J = 2.4 Hz, 1H), 7.73-7.64 (m, 1H), 7.39-7.34 (m, |
| | | | | 2H), 7.05 (d, J = 11.6 Hz, 1H), 6.95-6.88 (m, 1H), |
| | | | | 6.87-6.84 (m, 1H), 6.43 (s, 1H), 6.16-6.10 (m, 1H), |
| | | | | 4.62 (q, J = 7.2 Hz, 2H), 4.38-4.30 (m, 2H), 3.84 (s, |
| | | | | 3H), 3.79 (s, 4H), 3.74 (d, J = 3.2 Hz, 3H), 3.65 (t, J = |
| | | | | 5.6 Hz, 1H), 3.59 (t, J = 5.6 Hz, 1H), 3.25 (s, 4H), 3.09 |
| | | | | (q, J = 7.2 Hz, 2H), 2.36-2.26 (m, 2H) |
| I-614^{c, d} | IG | IE | 702.2 | 12.32-11.83 (m, 1H), 8.28 (d, J = 2.0 Hz, 1H), 8.17- |
| | | | | 8.06 (m, 1H), 7.79 (d, J = 2.4 Hz, 1H), 7.74-7.60 (m, |
| | | | | 2H), 7.37 (dd, J = 2.4, 10.4 Hz, 1H), 7.26-7.15 (m, |
| | | | | 1H), 6.67 (d, J = 2.8 Hz, 1H), 6.17-6.06 (m, 1H), 4.66- |
| | | | | 4.58 (m, 2H), 4.39-4.30 (m, 2H), 3.86-3.82 (m, |
| | | | | 6H), 3.80 (s, 4H), 3.67-3.58 (m, 2H), 3.26 (s, 4H), |
| | | | | 3.09 (q, J = 7.2 Hz, 2H), 2.37-2.26 (m, 2H) |
| I-615 | IC | IO | 669.2 | |
| I-616 | IC | IP | 697.2 | |
| I-617 | IC | IQ | 752.3 | |
| I-618 | IC | IR | 741.3 | 12.10-12.03 (m, 1H), 8.17-8.07 (m, 1H), 7.83-7.76 |
| | | | | (m, 1H), 7.73-7.65 (m, 1H), 7.41-7.34 (m, 1H), 7.27 |
| | | | | (dd, J = 3.6, 8.4 Hz, 1H), 6.99-6.90 (m, 1H), 6.67- |
| | | | | 6.58 (m, 2H), 6.49 (d, J = 2.0 Hz, 1H), 6.16-6.07 (m, |
| | | | | 1H), 4.66-4.59 (m, 2H), 4.39-4.30 (m, 2H), 4.08- |
| | | | | 4.01 (m, 3H), 3.85 (s, 3H), 3.80 (s, 4H), 3.68-3.59 (m, |
| | | | | 2H), 3.25 (s, 4H), 3.13-3.07 (m, 2H), 2.37-2.27 (m, |
| | | | | 2H), 1.37 (t, J = 6.8 Hz, 3H), 1.31 (t, J = 6.8 Hz, 2H), |
| | | | | 1.18-1.14 (m, 2H) |
| I-619^{d} | IJ | IM | 583.3 | 12.32 (s, 1H), 8.53 (d, J = 5.2 Hz, 1H), 8.44-8.38 (m, |
| | | | | 1H), 7.81-7.75 (m, 1H), 7.42 (d, J = 5.2 Hz, 1H), 7.29- |
| | | | | 7.23 (m, 1H), 6.98-6.87 (m, 2H), 6.35 (s, 1H), 6.21- |
| | | | | 6.14 (m, 1H), 4.35 (d, J = 6.4 Hz, 2H), 3.80 (s, 3H), |
| | | | | 3.77 (s, 4H), 3.63-3.58 (m, 2H), 3.41-3.33 (m, 2H), |
| | | | | 2.47-2.40 (m, 2H), 2.37 (d, J = 3.2 Hz, 2H), 2.34- |
| | | | | 2.17 (m, 2H), 2.08 (d, J = 4.4 Hz, 3H), 1.06-0.97 (m, 3H) |
| I-620 | IC | IS | 747.3 | |
| I-621 | IC | IN | 733.2 | |
| I-622 | IC | IT | 747.3 | |
| I-623 | IC | IU | 721.3 | 12.36-12.24 (m, 1H), 8.17-8.06 (m, 1H), 7.85-7.73 |
| | | | | (m, 3H), 7.73-7.66 (m, 1H), 7.42 (dd, J = 3.6, 8.0 Hz, |
| | | | | 1H), 7.37 (dd, J = 2.0, 10.0 Hz, 1H), 7.22-7.10 (m, |
| | | | | 1H), 6.94-6.82 (m, 1H), 6.25-6.12 (m, 1H), 4.64 (q, J = |
| | | | | 6.8 Hz, 2H), 4.45-4.34 (m, 2H), 4.02 (s, 3H), 3.87- |
| | | | | 3.77 (m, 7H), 3.70-3.61 (m, 2H), 3.28 (d, J = 2.0 Hz, |
| | | | | 4H), 3.14-3.08 (m, 2H), 2.44-2.37 (m, 3H), 2.36- |
| | | | | 2.25 (m, 2H) |
| I-624 | IC | IV | 707.3 | |
| I-625 | IC | IW | 733.3 | |
| I-626 | IC | IX | 730.3 | |
| I-627 | IC | IY | 704.3 | 12.39-12.28 (m, 1H), 9.00 (d, J = 1.9 Hz, 1H), 8.64- |
| | | | | 8.55 (m, 1H), 8.22-8.15 (m, 1H), 8.14-8.08 (m, 1H), |
| | | | | 7.99-7.93 (m, 1H), 7.81-7.72 (m, 3H), 7.71-7.62 (m, |
| | | | | 2H), 7.51 (dd, J = 4.8, 8.0 Hz, 1H), 7.37 (dd, J = 2.4, |
| | | | | 10.4 Hz, 1H), 7.25-7.15 (m, 1H), 6.91 (s, 1H), 6.25- |
| | | | | 6.17 (m, 1H), 4.63 (q, J = 7.2 Hz, 2H), 4.44-4.34 (m, |
| | | | | 2H), 3.88-3.79 (m, 7H), 3.69-3.60 (m, 2H), 3.27 (s, |
| | | | | 4H), 3.11 (q, J = 6.8 Hz, 2H), 2.38-2.28 (m, 2H) |
| I-628 | IC | IZ | 694.1 | |
| I-629 | IC | JB | 730.2 | 12.39-12.28 (m, 1H), 9.00 (d, J = 1.9 Hz, 1H), 8.64- |
| | | | | 8.55 (m, 1H), 8.22-8.15 (m, 1H), 8.14-8.08 (m, 1H), |
| | | | | 7.99-7.93 (m, 1H), 7.81-7.72 (m, 3H), 7.71-7.62 (m, |
| | | | | 2H), 7.51 (dd, J = 4.8, 8.0 Hz, 1H), 7.37 (dd, J = 2.4, |
| | | | | 10.4 Hz, 1H), 7.25-7.15 (m, 1H), 6.91 (s, 1H), 6.25- |
| | | | | 6.17 (m, 1H), 4.63 (q, J = 7.2 Hz, 2H), 4.44-4.34 (m, |
| | | | | 2H), 3.88-3.79 (m, 7H), 3.69-3.60 (m, 2H), 3.27 (s, |
| | | | | 4H), 3.11 (q, J = 6.8 Hz, 2H), 2.38-2.28 (m, 2H) |
| I-630 | IC | JC | 718.2 | 12.43 (s, 1H), 8.14-8.08 (m, 1H), 8.02 (d, J = 8.4 Hz, |
| | | | | 1H), 7.78-7.72 (m, 2H), 7.72-7.66 (m, 2H), 7.50- |
| | | | | 7.43 (m, 2H), 7.37-7.32 (m, 1H), 7.18-7.06 (m, 1H), |
| | | | | 6.38-6.31 (m, 1H), 6.24-6.17 (m, 1H), 4.67-4.58 (m, |
| | | | | 2H), 4.43-4.34 (m, 2H), 3.81 (s, 3H), 3.72 (s, 4H), |
| | | | | 3.67-3.58 (m, 2H), 3.25-3.14 (m, 4H), 3.11 (t, J = 7.6 |
| | | | | Hz, 2H), 2.72 (s, 3H), 2.37-2.26 (m, 2H) |
| I-631 | IC | JD | 736.3 | |
| I-632 | IC | JA | 682.2 | |
| I-633 | IC | JE | 718.2 | |
| I-634 | IC | JF | 692.3 | |
| I-635 | IC | JG | 718.2 | |
| I-636 | IC | JH | 718.2 | |
| I-637 | IC | JI | 694.2 | |
| I-638 | IC | JJ | 711.3 | |
| I-639 | IC | JK | 645.3 | |
| I-640 | IC | JL | 687.3 | |
| I-641 | IC | JM | 723.3 | |
| I-642 | IC | JN | 733.3 | |
| I-643 | IC | JO | 715.3 | |
| I-644 | IC | JP | 673.2 | |
| I-645 | IC | JQ | 693.3 | |
| I-646 | IC | JR | 682.3 | |
| I-647 | IC | JS | 730.2 | |
| I-648 | IC | JT | 692.2 | |
| I-649 | IC | JU | 718.2 | |
| I-650 | IC | JV | 736.2 | |
| I-651 | IC | JW | 720.2 | |
| I-652 | IC | JX | 712.3 | |
| I-653 | IC | JY | 720.3 | |
| I-654 | IC | JZ | 726.2 | |
| I-655 | IC | KA | 712.3 | |
| I-656 | IC | KB | 702.3 | |
| I-657 | IC | KC | 700.3 | |
| I-658 | IC | KD | 691.2 | 12.36-12.14 (m, 1H), 8.18-8.05 (m, 1H), 7.83-7.65 |
| | | | | (m, 2H), 7.45-7.29 (m, 4H), 7.03-6.89 (m, 1H), 6.52 |
| | | | | (s, 1H), 6.23-6.07 (m, 1H), 5.29 (d, J = 6.0 Hz, 1H), |
| | | | | 5.17-5.08 (m, 1H), 4.63 (q, J = 7.6 Hz, 2H), 4.43- |
| | | | | 4.30 (m, 2H), 3.87-3.75 (m, 7H), 3.69-3.57 (m, 2H), |
| | | | | 3.25 (s, 4H), 3.10 (q, J = 6.8 Hz, 2H), 2.81-2.67 (m, |
| | | | | 2H), 2.39-2.25 (m, 3H), 1.83-1.67 (m, 1H) |
| I-659 | IC | KE | 719.3 | |
| I-660 | IC | KF | 731.3 | |
| I-661 | IC | KG | 727.3 | |
| I-662 | KH | FH | 680.3 | 12.24 (d, J = 7.6 Hz, 1H), 7.78 (d, J = 2.4 Hz, 1H), 7.74- |
| | | | | 7.68 (m, 1H), 7.43-7.33 (m, 4H), 7.30-7.22 (m, |
| | | | | 2H), 6.90-6.78 (m, 1H), 6.28 (s, 1H), 6.21-6.10 (m, |
| | | | | 2H), 4.38-4.34 (m, 3H), 4.33-4.27 (m, 1H), 3.83 (s, |
| | | | | 3H), 3.75 (s, 4H), 3.65-3.55 (m, 2H), 3.36 (d, J = 1.6 |
| | | | | Hz, 3H), 3.22 (s, 4H), 2.96 (q, J = 7.2 Hz, 2H), 2.30- |
| | | | | 2.22 (m, 1H), 0.97 (t, J = 7.6 Hz, 3H) |
| I-663 | KH | DZ | 683.2 | 12.25 (d, J = 7.6 Hz, 1H), 8.50 (s, 1H), 8.30 (d, J = 4.8 |
| | | | | Hz, 1H), 7.79 (d, J = 2.4 Hz, 1H), 7.71 (dd, J = 2.0, |
| | | | | 11.2 Hz, 1H), 7.43-7.35 (m, 3H), 7.09-6.99 (m, 1H), |
| | | | | 6.52 (s, 1H), 6.22-6.12 (m, 2H), 4.39-4.36 (m, 2H), |
| | | | | 4.35-4.28 (m, 2H), 3.86 (d, J = 3.6 Hz, 3H), 3.84 (s, |
| | | | | 3H), 3.79 (s, 4H), 3.64 (t, J = 5.6 Hz, 1H), 3.58 (t, J = |
| | | | | 5.6 Hz, 1H), 3.26 (s, 4H), 3.00-2.97 (m, 1H), 2.97- |
| | | | | 2.93 (m, 1H), 2.33 (d, J = 2.0 Hz, 1H), 2.26 (d, J = 2.8 |
| | | | | Hz, 1H) |
| I-664 | KH | FQ | 683.3 | 12.26-12.15 (m, 1H), 8.50 (d, J = 5.6 Hz, 1H), 8.41 (s, |
| | | | | 1H), 7.79 (d, J = 2.4 Hz, 1H), 7.74-7.68 (m, 1H), 7.45- |
| | | | | 7.33 (m, 2H), 7.21 (d, J = 5.6 Hz, 1H), 7.04-6.92 (m, |
| | | | | 1H), 6.47 (s, 1H), 6.22-6.11 (m, 2H), 4.39-4.34 (m, |
| | | | | 3H), 4.34-4.28 (m, 1H), 3.84 (s, 3H), 3.83-3.77 (m, |
| | | | | 7H), 3.64 (t, J = 5.6 Hz, 1H), 3.57 (t, J = 5.6 Hz, 1H), |
| | | | | 3.25 (s, 4H), 2.97 (q, J = 7.2 Hz, 2H), 2.34-2.24 (m, 2H) |
| I-665 | KH | FH | 681.3 | 12.25 (br d, J = 5.6 Hz, 1H), 8.13-8.09 (m, 1H), 7.78 |
| | | | | (d, J = 2.4 Hz, 1H), 7.71-7.66 (m, 1H), 7.39-7.35 (m, |
| | | | | 3H), 7.30-7.24 (m, 2H), 6.91-6.81 (m, 1H), 6.29 (br |
| | | | | s, 1H), 6.18-6.13 (m, 1H), 4.62 (d, J = 7.2 Hz, 2H), |
| | | | | 4.39-4.32 (m, 2H), 3.84 (s, 3H), 3.76 (br s, 4H), 3.65 |
| | | | | (br t, J = 5.6 Hz, 1H), 3.60 (br t, J = 5.6 Hz, 1H), 3.23 |
| | | | | (br s, 4H), 3.12-3.08 (m, 2H), 2.48 (br s, 2H), 2.35 (br |
| | | | | d, J = 3.2 Hz, 2H), 0.98 (t, J = 7.6 Hz, 3H) |
| I-666 | KH | CG | 684.3 | 12.26 (br s, 1H), 8.51 (s, 1H), 8.31 (d, J = 4.8 Hz, 1H), |
| | | | | 8.14-8.09 (m, 1H), 7.79 (d, J = 2.4 Hz, 1H), 7.72- |
| | | | | 7.67 (m, 1H), 7.42-7.36 (m, 2H), 7.09-7.01 (m, 1H), |
| | | | | 6.53 (br s, 1H), 6.18-6.12 (m, 1H), 4.66-4.60 (m, |
| | | | | 2H), 4.39-4.31 (m, 2H), 3.86 (d, J = 3.2 Hz, 3H), 3.84 |
| | | | | (s, 3H), 3.80 (br s, 4H), 3.66 (br t, J = 5.6 Hz, 1H), 3.62- |
| | | | | 3.59 (m, 1H), 3.26 (br s, 4H), 3.10 (br d, J = 8.4 Hz, |
| | | | | 2H), 2.37-2.27 (m, 2H) |
| I-667 | KH | FV | 698.3 | 12.20-12.15 (m, 1H), 8.26 (s, 1H), 8.14-8.08 (m, |
| | | | | 1H), 7.79 (d, J = 2.4 Hz, 1H), 7.75-7.61 (m, 1H), 7.36 |
| | | | | (dd, J = 2.4, 10.3 Hz, 1H), 7.08 (s, 1H), 6.99-6.90 (m, |
| | | | | 1H), 6.46 (d, J = 2.0 Hz, 1H), 6.17-6.11 (m, 1H), 4.62 |
| | | | | (q, J = 7.6 Hz, 2H), 4.38-4.30 (m, 2H), 3.84 (s, 3H), |
| | | | | 3.83-3.81 (m, 1H), 3.80 (s, 3H), 3.79 (s, 2H), 3.78- |
| | | | | 3.76 (m, 1H), 3.65 (t, J = 5.6 Hz, 1H), 3.60 (t, J = 6.0 |
| | | | | Hz, 1H), 3.25 (s, 4H), 3.09 (q, J = 6.8 Hz, 2H), 2.52- |
| | | | | 2.51 (m, 3H), 2.36-2.26 (m, 2H) |
| I-668 | KH | HO | 696.1 | 12.38-12.33 (m, 1H), 8.57-8.54 (m, 1H), 8.38 (s, |
| | | | | 1H), 8.12-8.08 (m, 1H), 7.78 (d, J = 2.0 Hz, 1H), 7.70- |
| | | | | 7.66 (m, 1H), 7.49 (br d, J = 5.2 Hz, 1H), 7.36 (dd, J = |
| | | | | 2.0, 10.4 Hz, 1H), 6.96-6.86 (m, 1H), 6.33 (br s, 1H), |
| | | | | 6.17 (br d, J = 12.4 Hz, 1H), 4.61 (br d, J = 6.8 Hz, |
| | | | | 2H), 4.39-4.32 (m, 2H), 3.83 (s, 3H), 3.76 (br s, 4H), |
| | | | | 3.65 (br d, J = 5.6 Hz, 1H), 3.59 (br s, 1H), 3.23 (br s, |
| | | | | 4H), 3.11-3.07 (m, 2H), 2.88-2.83 (m, 1H), 2.34 (br |
| | | | | s, 1H), 2.27 (br s, 1H), 1.13-1.05 (m, 6H) |
| I-669 | KK | KI | 695.3 | 12.41-12.29 (m, 1H), 8.56 (d, J = 5.2 Hz, 1H), 8.38 (s, |
| | | | | 1H), 7.78 (d, J = 2.4 Hz, 1H), 7.74-7.67 (m, 1H), 7.50 |
| | | | | (d, J = 5.2 Hz, 1H), 7.44-7.33 (m, 2H), 6.96-6.85 (m, |
| | | | | 1H), 6.33 (d, J = 2.0 Hz, 1H), 6.23-6.09 (m, 2H), 4.39- |
| | | | | 4.29 (m, 4H), 3.83 (s, 3H), 3.76 (s, 4H), 3.66-3.55 |
| | | | | (m, 3H), 3.23 (s, 4H), 2.97 (q, J = 6.8 Hz, 2H), 2.88- |
| | | | | 2.82 (m, 1H), 2.26 (d, J = 4.0 Hz, 1H), 1.12 (d, J = 3.6 |
| | | | | Hz, 3H), 1.05 (d, J = 6.0 Hz, 3H) |
| I-670 | IC | KN | 731.2 | |
| I-671 | IC | KO | 719.2 | |
| I-672 | IC | KP | 718.2 | |
| I-673 | IC | KQ | 667.2 | |
| I-674 | IC | KR | 693.2 | |
| I-675 | IC | KS | 617.2 | |
| I-676 | IC | KT | 685.2 | |
| I-677 | IC | KU | 645.3 | |
| I-678 | IC | KV | 725.2 | |
| I-679 | IC | KW | 713.2 | 12.27-12.09 (m, 1H), 8.17-8.03 (m, 1H), 7.83-7.74 |
| | | | | (m, 1H), 7.73-7.63 (m, 1H), 7.39 (s, 2H), 7.38-7.23 |
| | | | | (m, 3H), 6.97-6.87 (m, 1H), 6.36 (s, 1H), 6.21-6.09 |
| | | | | (m, 1H), 4.67-4.58 (m, 2H), 4.41-4.30 (m, 2H), 3.87- |
| | | | | 3.74 (m, 7H), 3.67-3.57 (m, 2H), 3.24 (s, 4H), 3.10 |
| | | | | (q, J = 6.8 Hz, 2H), 2.90-2.81 (m, 2H), 2.38-2.26 (m, |
| | | | | 2H), 1.18-1.07 (m, 3H) |
| I-680 | IC | KX | 739.2 | |
| I-681 | IC | KY | 736.2 | |
| I-682 | IC | KZ | 720.2 | |
| I-683 | FG | LC | 693.3 | 8.54 (d, J = 5.2 Hz, 1H), 8.46-8.36 (m, 2H), 7.73 (d, J = |
| | | | | 2.0 Hz, 1H), 7.54 (d, J = 2.4 Hz, 1H), 7.46-7.34 (m, |
| | | | | 2H), 6.98-6.86 (m, 2H), 6.35 (d, J = 3.0 Hz, 1H), 6.24- |
| | | | | 6.12 (m, 2H), 4.39-4.29 (m, 4H), 3.81 (s, 3H), 3.78 |
| | | | | (s, 7H), 3.66-3.58 (m, 2H), 3.46 (d, J = 10.8 Hz, 2H), |
| | | | | 3.16 (s, 4H), 3.00-2.95 (m, 2H), 2.34 (s, 2H), 1.02 (t, J = |
| | | | | 7.6 Hz, 3H) |
| I-684 | KJ | LC | 693.3 | 8.61 (s, 1H), 8.48 (d, J = 4.8 Hz, 1H), 8.41 (s, 1H), 7.73 |
| | | | | (d, J = 2.0 Hz, 1H), 7.54 (d, J = 2.4 Hz, 1H), 7.45-7.35 |
| | | | | (m, 1H), 7.29 (d, J = 4.8 Hz, 1H), 6.98-6.87 (m, 2H), |
| | | | | 6.38 (d, J = 2.8 Hz, 1H), 6.22-6.14 (m, 2H), 4.38- |
| | | | | 4.30 (m, 4H), 3.81 (s, 3H), 3.78 (s, 7H), 3.64-3.58 (m, |
| | | | | 2H), 3.16 (s, 4H), 2.97 (d, J = 8.0 Hz, 2H), 2.56 (d, J = |
| | | | | 7.6 Hz, 2H), 2.34 (s, 2H), 0.98 (t, J = 7.6 Hz, 3H) |
| I-685 | LD | KI | 695.3 | 12.47-12.31 (m, 1H), 8.76-8.67 (m, 1H), 8.48-8.44 |
| | | | | (m, 1H), 7.78 (d, J = 2.4 Hz, 1H), 7.74-7.68 (m, 1H), |
| | | | | 7.43-7.37 (m, 1H), 7.35 (s, 1H), 7.24 (br d, J = 4.4 Hz, |
| | | | | 1H), 6.93-6.83 (m, 1H), 6.37-6.34 (m, 1H), 6.21- |
| | | | | 6.12 (m, 2H), 4.36 (br t, J = 6.8 Hz, 3H), 4.31 (br d, J = |
| | | | | 15.2 Hz, 1H), 3.83 (s, 3H), 3.76 (br s, 4H), 3.63 (br s, |
| | | | | 1H), 3.57 (br t, J = 5.6 Hz, 1H), 3.23 (br s, 4H), 3.00- |
| | | | | 2.94 (m, 2H), 2.93-2.87 (m, 1H), 2.33 (br s, 1H), 2.30- |
| | | | | 2.21 (m, 1H), 1.15 (br s, 6H) |
| I-686 | FG | KI | 681.3 | 12.41-12.27 (m, 1H), 8.53 (d, J = 5.2 Hz, 1H), 8.40 (br |
| | | | | s, 1H), 7.78 (d, J = 2.4 Hz, 1H), 7.73-7.67 (m, 1H), |
| | | | | 7.44-7.37 (m, 2H), 7.37-7.34 (m, 1H), 6.97-6.87 (m, |
| | | | | 1H), 6.34 (br s, 1H), 6.21-6.13 (m, 2H), 4.36 (br t, J = |
| | | | | 6.8 Hz, 3H), 4.33 (br s, 1H), 3.83 (s, 3H), 3.76 (br s, |
| | | | | 4H), 3.64 (br t, J = 5.6 Hz, 1H), 3.58-3.56 (m, 1H), |
| | | | | 3.23 (br s, 5H), 2.97 (q, J = 6.8 Hz, 2H), 2.49-2.45 (m, |
| | | | | 1H), 2.33 (br s, 1H), 2.26 (br s, 1H), 1.01 (t, J = 7.6 Hz, 3H) |
| I-687 | KH | M | 682.1 | 12.15-12.08 (m, 1H), 7.78 (d, J = 2.4 Hz, 1H), 7.74- |
| | | | | 7.69 (m, 1H), 7.43-7.33 (m, 4H), 7.15 (d, J = 8.4 Hz, |
| | | | | 1H), 7.05 (t, J = 7.6 Hz, 1H), 6.96-6.88 (m, 1H), 6.43 |
| | | | | (s, 1H), 6.22-6.16 (m, 1H), 6.14-6.09 (m, 1H), 4.39- |
| | | | | 4.35 (m, 3H), 4.34-4.27 (m, 1H), 3.83 (s, 3H), 3.79 (s, |
| | | | | 4H), 3.73-3.71 (m, 3H), 3.66-3.56 (m, 2H), 3.25 (s, |
| | | | | 4H), 3.00-2.97 (m, 1H), 2.97-2.93 (m, 1H), 2.32 (d, J = |
| | | | | 1.6 Hz, 2H) |
| I-688 | KJ | KI | 681.2 | 12.45-12.25 (m, 1H), 8.60 (s, 1H), 8.47 (d, J = 4.8 Hz, |
| | | | | 1H), 7.78 (d, J = 2.4 Hz, 1H), 7.75-7.66 (m, 1H), 7.45- |
| | | | | 7.32 (m, 2H), 7.28 (d, J = 4.8 Hz, 1H), 6.99-6.85 (m, |
| | | | | 1H), 6.37 (s, 1H), 6.22-6.11 (m, 2H), 4.38-4.28 (m, |
| | | | | 4H), 3.83 (s, 3H), 3.76 (s, 4H), 3.65-3.55 (m, 2H), |
| | | | | 3.23 (s, 4H), 3.00-2.93 (m, 2H), 2.58-2.53 (m, 2H), |
| | | | | 2.34-2.24 (m, 2H), 0.97 (t, J = 7.6 Hz, 3H) |
| I-689 | IF | IG | 682.3 | 12.35 (br s, 1H), 8.60 (s, 1H), 8.47 (d, J = 5.2 Hz, 1H), |
| | | | | 8.12-8.09 (m, 1H), 7.78 (d, J = 2.0 Hz, 1H), 7.70- |
| | | | | 7.66 (m, 1H), 7.36 (dd, J = 2.4, 10.4 Hz, 1H), 7.29- |
| | | | | 7.26 (m, 1H), 6.98-6.88 (m, 1H), 6.39-6.36 (m, 1H), |
| | | | | 6.19-6.13 (m, 1H), 4.61 (br d, J = 6.8 Hz, 2H), 4.38- |
| | | | | 4.31 (m, 2H), 3.83 (s, 3H), 3.76 (br s, 4H), 3.64 (br t, J = |
| | | | | 5.2 Hz, 1H), 3.59 (br s, 1H), 3.23 (br s, 4H), 3.09 (br |
| | | | | d, J = 7.2 Hz, 2H), 2.40-2.38 (m, 2H), 2.33 (br d, J = |
| | | | | 1.6 Hz, 1H), 2.28-2.25 (m, 1H), 0.97 (t, J = 7.6 Hz, 3H) |
| I-690 | LF | DE | 715.2 | 12.53-12.28 (m, 1H), 8.49 (s, 1H), 8.17-8.03 (m, |
| | | | | 1H), 7.80 (dd, J = 1.2, 4.8 Hz, 1H), 7.74-7.63 (m, 1H), |
| | | | | 7.52-7.42 (m, 2H), 7.38-7.32 (m, 1H), 7.27 (dd, J = |
| | | | | 1.2, 8.0 Hz, 1H), 7.22-7.10 (m, 1H), 6.92 (dd, J = 4.8, |
| | | | | 8.0 Hz, 1H), 6.81-6.70 (m, 1H), 6.23-6.13 (m, 1H), |
| | | | | 4.68-4.57 (m, 2H), 4.42-4.29 (m, 2H), 3.82 (br s, |
| | | | | 4H), 3.81 (s, 3H), 3.69-3.59 (m, 2H), 3.39-3.34 (m, |
| | | | | 4H), 3.15-3.04 (m, 2H), 2.41-2.27 (m, 1H), 2.38- |
| | | | | 2.27 (m, 1H) |
| I-691 | IC | LK | 752.2 | |
| I-692 | LN | LO | 653.4 | 12.57 (br d, J = 3.6 Hz, 1H), 8.13 (d, J = 5.6 Hz, 1H), |
| | | | | 8.09 (s, 1H), 7.79 (dd, J = 1.2, 4.8 Hz, 1H), 7.72-7.64 |
| | | | | (m, 1H), 7.40-7.31 (m, 1H), 7.27 (dd, J = 1.2, 8.0 Hz, |
| | | | | 1H), 7.16-6.97 (m, 2H), 6.92 (dd, J = 4.8, 8.0 Hz, 1H), |
| | | | | 6.44 (s, 1H), 6.20 (d, J = 11.6 Hz, 1H), 4.62 (q, J = 6.4 |
| | | | | Hz, 2H), 4.43-4.28 (m, 2H), 3.80 (s, 3H), 3.79-3.72 |
| | | | | (m, 4H), 3.67-3.56 (m, 2H), 3.40-3.33 (m, 4H), 3.13- |
| | | | | 3.06 (m, 2H), 2.57-2.52 (m, 2H), 2.40-2.19 (m, |
| | | | | 2H), 1.10 (t, J = 6.8 Hz, 3H) |
| I-693 | LR | B | 619.2 | |
| I-694 | LS | B | 617.2 | |
| I-695 | LT | B | 661.3 | |
| I-696 | LX | CH | 603.3 | 12.37 (s, 1H), 8.94-8.88 (m, 1H), 8.83 (d, J = 3.2 Hz, |
| | | | | 1H), 7.82-7.75 (m, 2H), 7.39 (dd, J = 2.4, 10.4 Hz, |
| | | | | 1H), 7.21-7.11 (m, 1H), 6.68 (s, 1H), 6.18 (s, 1H), |
| | | | | 4.37-4.30 (m, 2H), 4.07 (s, 3H), 3.84 (s, 3H), 3.80 (s, |
| | | | | 4H), 3.65-3.57 (m, 2H), 3.27 (s, 4H), 2.39-2.25 (m, |
| | | | | 2H), 2.12-2.05 (m, 3H) |
| I-697 | LY | CH | 603.2 | 12.45 (s, 1H), 8.79 (s, 1H), 8.66-8.59 (m, 1H), 8.13- |
| | | | | 8.06 (m, 1H), 7.79 (d, J = 2.4 Hz, 1H), 7.38 (dd, J = |
| | | | | 2.0, 10.4 Hz, 1H), 7.29-7.19 (m, 1H), 6.69 (s, 1H), |
| | | | | 6.18 (s, 1H), 4.35 (s, 2H), 4.00 (s, 3H), 3.84 (s, 3H), |
| | | | | 3.80 (s, 4H), 3.62 (td, J = 5.6, 11.2 Hz, 2H), 3.27 (s, |
| | | | | 4H), 2.40-2.25 (m, 2H), 2.12-2.04 (m, 3H) |
| I-698 | MC | B | 710.5 | 8.43 (s, 1H), 8.14-8.10 (m, 1H), 7.82 (dd, J = 1.6, 4.8 |
| | | | | Hz, 1H), 7.72-7.66 (m, 1H), 7.29 (dd, J = 1.6, 8.0 Hz, |
| | | | | 1H), 6.96-6.89 (m, 2H), 6.85-6.78 (m, 1H), 6.06 (br |
| | | | | s, 1H), 4.63 (q, J = 6.4 Hz, 2H), 4.35-4.27 (m, 2H), |
| | | | | 3.83 (s, 6H), 3.68-3.58 (m, 3H), 3.38 (br s, 5H), 3.13- |
| | | | | 3.07 (m, 2H), 2.93 (br s, 5H), 2.36-2.25 (m, 2H), 1.82 |
| | | | | (br d, J = 12.8 Hz, 2H), 1.70 (br s, 6H), 1.45 (br s, 2H), |
| | | | | 1.39-1.30 (m, 2H) |
| I-699 | MG | MJ | 601.3 | 1.03 (m, 3 H) 2.04-2.11 (m, 3 H) 2.24-2.41 (m, 2 H) |
| | | | | 2.53 (br d, J = 7.6 Hz, 2 H) 3.07 (br s, 4 H) 3.58-3.64 |
| | | | | (m, 2 H) 3.79 (br s, 4 H) 3.85-3.88 (m, 3 H) 4.31- |
| | | | | 4.38 (m, 2 H) 6.14-6.23 (m, 1 H) 6.39 (d, J = 2.4 Hz, 1 |
| | | | | H) 6.89-7.01 (m, 1 H) 7.18 (m, 1 H) 7.53 (br d, J = 4.8 |
| | | | | Hz, 1 H) 7.72 (d, J = 2.4 Hz, 1 H) 8.09-8.17 (m, 1 H) |
| | | | | 8.48 (s, 1 H) 8.56-8.61 (m, 1 H) 12.37 (s, 1 H) |
| I-700 | MK | MM | 593.1 | 11.89 (s, 1H), 8.04 (s, 1H), 7.65 (s, 1H), 7.44-7.34 (m, |
| | | | | 2H), 7.32-7.25 (m, 2H), 7.22 (s, 1H), 6.84 (s, 1H), |
| | | | | 6.57 (s, 1H), 6.16 (s, 1H), 4.82 (s, 2H), 4.70 (s, 2H), |
| | | | | 4.67-4.60 (m, 2H), 4.37 (s, 2H), 3.77 (s, 3H), 3.63 (s, |
| | | | | 2H), 3.08 (t, J = 6.8 Hz, 2H), 2.57-2.52 (m, 2H), 2.34 |
| | | | | (d, J = 3.2 Hz, 2H), 1.01 (t, J = 7.4 Hz, 3H) |
| I-701 | MN | FO | 515.2 | 12.21 (d, J = 1.6 Hz, 1H), 8.56-8.42 (m, 2H), 7.31- |
| | | | | 7.19 (m, 2H), 7.08-6.94 (m, 1H), 6.83-6.76 (m, 1H), |
| | | | | 6.22-6.11 (m, 1H), 5.03 (s, 1H), 4.87-4.82 (m, 1H), |
| | | | | 4.79 (s, 1H), 4.63 (s, 1H), 4.35 (d, J = 9.2 Hz, 2H), 3.92- |
| | | | | 3.80 (m, 5H), 3.75 (s, 1H), 3.65-3.59 (m, 2H), 2.41- |
| | | | | 2.26 (m, 2H), 2.09 (d, J = 6.0 Hz, 3H) |
| I-702 | FG | MO | 513.2 | 12.39-12.26 (m, 1H), 8.59 (s, 1H), 8.46-8.42 (m, |
| | | | | 1H), 7.45 (d, J = 4.4 Hz, 1H), 7.29-7.20 (m, 1H), 6.90 |
| | | | | (d, J = 6.4 Hz, 1H), 6.70-6.62 (m, 1H), 6.24 (s, 1H), |
| | | | | 5.01-4.92 (m, 1H), 4.81-4.76 (m, 2H), 4.68-4.53 (m, |
| | | | | 1H), 4.37 (s, 2H), 3.82-3.71 (m, 3H), 3.65-3.58 (m, |
| | | | | 2H), 2.41-2.36 (m, 3H), 2.09 (d, J = 4.4 Hz, 3H), 1.25- |
| | | | | 1.01 (m, 4H) |
| I-703 | MP | IG | 696.3 | 12.37 (br d, J = 4.0 Hz, 1 H) 8.71 (s, 1 H) 8.46 (d, J = 4.8 |
| | | | | Hz, 1 H) 8.06-8.14 (m, 1 H) 7.78 (d, J = 2.4 Hz, 1 H) |
| | | | | 7.65-7.72 (m, 1 H) 7.36 (m, 1 H) 7.20-7.27 (m, 1 H) |
| | | | | 6.81-6.99 (m, 1 H) 6.35 (br s, 1 H) 6.09-6.24 (m, 1 |
| | | | | H) 4.62 (m, 2 H) 4.29-4.42 (m, 2 H) 3.83 (s, 3 H) 3.76 |
| | | | | (br s, 4 H) 3.56-3.68 (m, 2 H) 3.23 (br s, 4 H) 3.09 (m, |
| | | | | 2 H) 2.84-2.95 (m, 1 H) 2.22-2.39 (m, 2 H) 1.15 (br |
| | | | | s, 6 H) |
| I-704 | FG | MM | 594.3 | 12.26-11.72 (m, 1H), 8.54 (d, J = 5.2 Hz, 1H), 8.44 (s, |
| | | | | 1H), 8.21 (br s, 1H), 8.04 (s, 1H), 7.64 (s, 1H), 7.42 (br |
| | | | | d, J = 5.2 Hz, 1H), 7.22 (s, 1H), 6.90 (br s, 1H), 6.62 |
| | | | | (br d, J = 2.4 Hz, 1H), 6.18 (br s, 1H), 4.99-4.68 (m, |
| | | | | 4H), 4.66-4.61 (m, 2H), 4.37 (br s, 2H), 3.77 (br s, |
| | | | | 3H), 3.63 (br s, 2H), 3.07 (br s, 2H), 2.60-2.51 (m, |
| | | | | 2H), 2.34 (br s, 2H), 1.05 (br t, J = 7.2 Hz, 3H) |
| I-705 | KJ | MO | 513.2 | (CDCl₃) 9.76-9.67 (m, 1H), 8.72-8.53 (m, 2H), 7.31 |
| | | | | (s, 1H), 6.94-6.90 (m, 1H), 6.63-6.52 (m, 1H), 6.19 |
| | | | | (d, J = 1.6 Hz, 1H), 4.86 (s, 3H), 4.82-4.77 (m, 1H), |
| | | | | 4.50 (s, 1H), 4.38 (s, 1H), 3.89 (s, 3H), 3.80 (t, J = 4.4 |
| | | | | Hz, 1H), 3.66 (s, 1H), 2.68-2.55 (m, 2H), 2.47-2.36 |
| | | | | (m, 2H), 2.19 (s, 3H), 1.08 (d, J = 6.4 Hz, 3H) |
| I-706 | KJ | MR | 601.3 | 12.35 (s, 1H), 8.60 (s, 1H), 8.47 (d, J = 4.8 Hz, 1H), |
| | | | | 7.78 (s, 1H), 7.36 (dd, J = 2.4, 10.4 Hz, 1H), 7.28 (d, J = |
| | | | | 4.8 Hz, 1H), 7.01-6.85 (m, 1H), 6.38 (d, J = 2.8 Hz, |
| | | | | 1H), 6.22-6.12 (m, 1H), 4.40-4.29 (m, 2H), 3.83 (s, |
| | | | | 3H), 3.80-3.70 (m, 4H), 3.65-3.56 (m, 2H), 3.28- |
| | | | | 3.18 (m, 4H), 2.60-2.51 (m, 2H), 2.42-2.22 (m, 2H), |
| | | | | 2.07 (d, J = 5.6 Hz, 3H), 0.98 (d, J = 6.8 Hz, 3H) |
| I-707 | NA | IG | 696.3 | 12.29 (br d, J = 4.4 Hz, 1H), 8.34 (s, 1H), 8.13-8.08 |
| | | | | (m, 1H), 7.78 (d, J = 2.4 Hz, 1H), 7.75-7.62 (m, 1H), |
| | | | | 7.36 (dd, J = 2.4, 10.4 Hz, 1H), 7.25 (s, 1H), 6.97-6.86 |
| | | | | (m, 1H), 6.40 (s, 1H), 6.19-6.14 (m, 1H), 4.62 (q, J = |
| | | | | 7.2 Hz, 2H), 4.39-4.31 (m, 2H), 3.83 (s, 3H), 3.77 (s, |
| | | | | 4H), 3.64 (t, J = 6.0 Hz, 1H), 3.59 (t, J = 6.0 Hz, 1H), |
| | | | | 3.24 (s, 4H), 3.12-3.07 (m, 2H), 2.81-2.75 (m, 2H), |
| | | | | 2.35-2.25 (m, 2H), 2.15 (s, 3H), 1.28 (t, J = 7.6 Hz, 3H) |
| I-708 | NC | NF | 617.4 | 12.33 (s, 1 H) 8.54 (d, J = 5.2 Hz, 1 H) 8.42 (s, 1 H) 7.78 |
| | | | | (d, J = 2.4 Hz, 1 H) 7.44 (d, J = 5.2 Hz, 1 H) 7.36 (m, 1 |
| | | | | H) 6.89 (d, J = 6.0 Hz, 1 H) 6.35 (br s, 1 H) 6.17 (br s, 1 |
| | | | | H) 4.30 (br s, 2 H) 3.83 (s, 3 H) 3.76 (br s, 4 H) 3.64 (s, |
| | | | | 3 H) 3.55 (br t, J = 5.8 Hz, 2 H) 3.24 (br s, 4 H) 2.44- |
| | | | | 2.49 (m, 2 H) 2.30 (br d, J = 3.2 Hz, 2 H) 1.01 (t, J = 7.6 |
| | | | | Hz, 3 H) |
| I-709 | MN | CG | 515.2 | 12.25 (br s, 1H), 8.53 (d, J = 3.1 Hz, 1H), 8.33-8.31 |
| | | | | (m, 1H), 7.46-7.42 (m, 1H), 7.23 (s, 1H), 7.10-7.01 |
| | | | | (m, 1H), 6.85 (br s, 1H), 6.19-6.13 (m, 1H), 5.03 (s, |
| | | | | 1H), 4.85 (s, 1H), 4.78 (s, 1H), 4.63 (s, 1H), 4.36-4.32 |
| | | | | (m, 2H), 3.94-3.80 (m, 5H), 3.75 (s, 1H), 3.64-3.59 |
| | | | | (m, 2H), 2.38 (br d, J = 1.0 Hz, 1H), 2.27 (br d, J = 2.0 |
| | | | | Hz, 1H), 2.09-2.06 (m, 3H) |
| I-710 | NC | NE | 645.5 | 12.31 (s, 1H), 8.53 (d, J = 5.2 Hz, 1H), 8.41 (s, 1H), |
| | | | | 7.78 (d, J = 2.4 Hz, 1H), 7.42 (d, J = 5.2 Hz, 1H), 7.35 |
| | | | | (dd, J = 2.4, 10.4 Hz, 1H), 6.98-6.86 (m, 1H), 6.35 (br |
| | | | | s, 1H), 6.21-6.12 (m, 1H), 4.48-4.39 (m, 1H), 4.38- |
| | | | | 4.32 (m, 2H), 3.83 (s, 3H), 3.80-3.71 (m, 4H), 3.67- |
| | | | | 3.56 (m, 2H), 3.47-3.35 (m, 2H), 3.27-3.16 (m, 4H), |
| | | | | 2.42 (br d, J = 7.6 Hz, 2H), 2.38-2.23 (m, 2H), 1.74- |
| | | | | 1.59 (m, 2H), 1.43-1.13 (m, 2H), 1.06-0.97 (m, 3H) |
| I-711 | MT | MV | 598.3 | 12.29 (s, 1H), 8.92 (d, J = 5.2 Hz, 1H), 8.87-8.79 (m, |
| | | | | 1H), 8.12 (d, J = 9.6 Hz, 1H), 7.81 (dd, J = 3.6, 6.8 Hz, |
| | | | | 1H), 7.70 (d, J = 9.6 Hz, 1H), 7.33-7.22 (m, 1H), 7.22- |
| | | | | 7.16 (m, 1H), 6.96 (d, J = 4.4 Hz, 1H), 5.04 (s, 2H), |
| | | | | 4.85 (s, 1H), 4.79 (s, 1H), 4.62 (q, J = 6.0 Hz, 3H), 4.50 |
| | | | | (d, J = 12.0 Hz, 1H), 4.15 (s, 1H), 4.09 (s, 2H), 3.92 (d, |
| | | | | J = 12.4 Hz, 1H), 3.81 (s, 1H), 3.76 (s, 1H), 3.32-3.10 |
| | | | | (m, 1H), 3.08-2.99 (m, 3H), 2.80-2.55 (m, 1H), 1.96- |
| | | | | 1.84 (m, 2H), 1.78 (d, J = 12.8 Hz, 1H), 1.58-1.41 |
| | | | | (m, 1H) |
| I-712 | KK | MM | 608.3 | 12.31-11.79 (m, 1H), 8.58 (d, J = 5.2 Hz, 1H), 8.41 (s, |
| | | | | 1H), 8.04 (s, 1H), 7.64 (s, 1H), 7.48 (d, J = 5.2 Hz, 1H), |
| | | | | 7.22 (s, 1H), 6.89 (br s, 1H), 6.60 (d, J = 2.8 Hz, 1H), |
| | | | | 6.18 (br s, 1H), 5.06-4.67 (m, 4H), 4.65 (br t, J = 6.8 |
| | | | | Hz, 2H), 4.38 (br s, 2H), 3.77 (br s, 3H), 3.64 (br s, |
| | | | | 2H), 3.12-3.10 (m, 2H), 2.95-2.89 (m, 1H), 2.39- |
| | | | | 2.29 (m, 2H), 1.13 (br d, J = 5.6 Hz, 6H) |
| I-713 | IC | DQ | 684.2 | 12.19 (d, J = 4.4 Hz, 1H), 8.23 (d, J = 4.8 Hz, 1H), 8.14- |
| | | | | 8.04 (m, 1H), 7.82-7.76 (m, 2H), 7.72-7.64 (m, |
| | | | | 1H), 7.36 (dd, J = 2.4, 10.4 Hz, 1H), 7.12 (dd, J = 5.2, |
| | | | | 7.2 Hz, 1H), 7.05-6.97 (m, 1H), 6.50 (s, 1H), 6.18- |
| | | | | 6.11 (m, 1H), 4.62 (q, J = 7.2 Hz, 2H), 4.39-4.30 (m, |
| | | | | 2H), 3.85-3.82 (m, 6H), 3.79 (s, 4H), 3.65 (t, J = 5.6 |
| | | | | Hz, 1H), 3.60 (t, J = 5.6 Hz, 1H), 3.26 (s, 4H), 3.09 (q, |
| | | | | J = 7.2 Hz, 2H), 2.37-2.27 (m, 2H) |
| I-714 | NJ | DZ | 529.1 | 12.24 (br d, J = 2.4 Hz, 1H), 8.53 (br s, 1H), 8.32 (br d, |
| | | | | J = 4.4 Hz, 1H), 7.43 (br d, J = 5.2 Hz, 1H), 7.34-7.20 |
| | | | | (m, 1H), 7.01 (s, 1H), 6.86 (br d, J = 13.2 Hz, 1H), 6.16 |
| | | | | (br s, 1H), 5.07 (s, 1H), 4.83 (br d, J = 14.0 Hz, 2H), |
| | | | | 4.67-4.55 (m, 1H), 4.42-4.26 (m, 2H), 4.17-4.01 (m, |
| | | | | 2H), 3.91 (d, J = 18.4 Hz, 3H), 3.69-3.52 (m, 2H), |
| | | | | 2.43-2.21 (m, 2H), 2.14-2.01 (m, 3H), 1.34 (td, J = |
| | | | | 7.2, 14.8 Hz, 3H) |
| I-715 | NL | NK | 529.1 | 12.13-12.25 (m, 1 H), 8.52 (d, J = 5.6 Hz, 1 H), 8.42- |
| | | | | 8.48 (m, 1 H), 8.30 (s, 1 H), 7.21-7.32 (m, 2 H), 6.93- |
| | | | | 7.05 (m, 1 H), 6.82 (br d, J = 17.2 Hz, 1 H), 6.17 (br s, 1 |
| | | | | H), 5.07 (s, 1 H), 4.82 (br d, J = 11.6 Hz, 2 H), 4.62 (s, 1 |
| | | | | H), 4.34 (br d, J = 9.2 Hz, 2 H), 4.01-4.16 (m, 2 H), |
| | | | | 3.87 (d, J = 16.0 Hz, 3 H), 3.55-3.66 (m, 2 H), 2.38 (br |
| | | | | s, 1 H), 2.27 (br s, 1 H), 2.08 (d, J = 6.0 Hz, 3 H), 1.26- |
| | | | | 1.42 (m, 3 H) |
| I-716 | NS | DQ | 632.2 | 8.31 (d, J = 4.4 Hz, 1H), 8.11 (s, 1H), 7.87-7.81 (m, |
| | | | | 2H), 7.69 (s, 1H), 7.27-7.11 (m, 3H), 6.81-6.60 (m, |
| | | | | 1H), 6.05-5.98 (m, 1H), 4.75 (d, J = 10.0 Hz, 2H), |
| | | | | 4.63 (t, J = 6.8 Hz, 2H), 4.59 (s, 2H), 4.34-4.11 (m, |
| | | | | 2H), 3.82 (s, 3H), 3.80 (s, 1H), 3.70 (s, 2H), 3.67-3.56 |
| | | | | (m, 2H), 3.56-3.44 (m, 2H), 3.15-3.08 (m, 2H), 2.35- |
| | | | | 2.25 (m, 2H), 0.52 (s, 1H), 0.13-0.06 (m, 2H), −0.25 |
| | | | | (d, J = 1.6 Hz, 2H) |
| I-717 | NS | FO | 632.2 | 8.58 (d, J = 5.6 Hz, 1H), 8.45 (d, J = 5.6 Hz, 1H), 8.16 |
| | | | | (s, 1H), 8.11 (s, 1H), 7.86 (d, J = 6.4 Hz, 1H), 7.69 (s, |
| | | | | 1H), 7.29-7.09 (m, 3H), 6.81-6.60 (m, 1H), 6.08- |
| | | | | 5.95 (m, 1H), 4.75 (d, J = 10.4 Hz, 2H), 4.66-4.57 (m, |
| | | | | 4H), 4.34-4.11 (m, 2H), 3.81 (s, 4H), 3.70 (s, 2H), |
| | | | | 3.65-3.49 (m, 4H), 3.14-3.07 (m, 2H), 2.35-2.24 (m, |
| | | | | 2H), 0.51 (dd, J = 2.4, 3.2 Hz, 1H), 0.14-0.05 (m, |
| | | | | 2H), −0.25 (s, 2H) |
| I-718 | NS | IZ | 642.2 | 8.52 (d, J = 4.4 Hz, 1H), 8.11 (s, 1H), 7.91-7.87 (m, |
| | | | | 1H), 7.79-7.75 (m, 1H), 7.69 (s, 1H), 7.30-7.15 (m, |
| | | | | 3H), 6.83-6.63 (m, 1H), 6.05-5.97 (m, 1H), 4.76 (d, J = |
| | | | | 13.1 Hz, 2H), 4.65-4.59 (m, 4H), 4.37-4.10 (m, |
| | | | | 2H), 3.80-3.69 (m, 3H), 3.64-3.48 (m, 4H), 3.10 (t, J = |
| | | | | 6.8 Hz, 2H), 2.34-2.25 (m, 2H), 1.68-1.61 (m, 1H), |
| | | | | 1.04 (d, J = 3.2 Hz, 1H), 0.96 (s, 1H), 0.82-0.74 (m, |
| | | | | 2H), 0.55 (d, J = 6.4 Hz, 1H), 0.08 (d, J = 5.2 Hz, |
| | | | | 2H), −0.19-−0.28 (m, 2H) |
| I-719 | NX | FO | 668.2 | 10.77 (br s, 1H), 8.54 (br s, 1H), 8.42 (br d, J = 5.6 Hz, |
| | | | | 1H), 8.08 (d, J = 11.6 Hz, 1H), 7.79 (d, J = 2.4 Hz, 1H), |
| | | | | 7.69 (d, J = 4.4 Hz, 1H), 7.60 (d, J = 4.8 Hz, 1H), 7.36 |
| | | | | (dd, J = 2.4, 10.4 Hz, 1H), 7.22 (dd, J = 5.6, 10.4 Hz, |
| | | | | 1H), 7.03 (dd, J = 1.2, 7.6 Hz, 1H), 6.34 (dd, J = 1.2, |
| | | | | 8.8 Hz, 1H), 4.63-4.56 (m, 2H), 4.50 (br d, J = 11.2 |
| | | | | Hz, 1H), 4.42-4.37 (m, 1H), 3.83-3.82 (m, 1H), 3.85- |
| | | | | 3.81 (m, 4H), 3.79 (s, 2H), 3.68 (br s, 4H), 3.24 (br s, |
| | | | | 4H), 3.20-3.10 (m, 1H), 3.07-3.00 (m, 2H), 2.89- |
| | | | | 2.82 (m, 1H), 2.70-2.54 (m, 1H), 2.13-2.04 (m, 1H), |
| | | | | 1.80-1.71 (m, 2H), 1.49-1.35 (m, 1H) |
| I-720 | NX | IZ | 678.3 | 10.97-10.88 (m, 1H), 8.56-8.47 (m, 1H), 8.08 (d, J = |
| | | | | 12.0 Hz, 1H), 7.79 (d, J = 2.4 Hz, 1H), 7.72-7.60 (m, |
| | | | | 3H), 7.40-7.33 (m, 1H), 7.30-7.24 (m, 1H), 7.00 (s, |
| | | | | 1H), 6.43-6.35 (m, 1H), 4.64-4.56 (m, 2H), 4.55- |
| | | | | 4.31 (m, 1H), 4.09-3.85 (m, 1H), 3.84 (s, 3H), 3.68 (s, |
| | | | | 4H), 3.25 (s, 4H), 3.14-2.92 (m, 3H), 2.88-2.77 (m, |
| | | | | 1H), 2.71-2.53 (m, 1H), 2.14-2.03 (m, 1H), 1.81- |
| | | | | 1.67 (m, 3H), 1.50-1.33 (m, 1H), 1.15-0.94 (m, 2H), |
| | | | | 0.80-0.70 (m, 2H) |
| I-721 | NY | DF | 580.2 | 10.78 (s, 1H), 8.55 (dd, J = 2.4, 5.6 Hz, 1H), 8.46-8.41 |
| | | | | (m, 1H), 8.08 (d, J = 11.2 Hz, 1H), 7.79-7.74 (m, 1H), |
| | | | | 7.69 (d, J = 5.2 Hz, 1H), 7.26-7.15 (m, 3H), 6.35 (d, J = |
| | | | | 8.0 Hz, 1H), 4.74 (s, 2H), 4.63 (s, 4H), 4.53-4.00 |
| | | | | (m, 2H), 3.83 (s, 2H), 3.79 (s, 3H), 3.68 (s, 1H), 3.19 |
| | | | | (dd, J = 7.2, 16.8 Hz, 1H), 3.09-3.02 (m, 2H), 2.99- |
| | | | | 2.86 (m, 1H), 2.79-2.58 (m, 1H), 2.14-2.05 (m, 1H), |
| | | | | 1.80-1.70 (m, 2H), 1.51-1.37 (m, 1H) |
| I-722 | NY | IZ | 590.1 | 10.99-10.88 (m, 1H), 8.56-8.43 (m, 1H), 8.14-8.00 |
| | | | | (m, 1H), 7.82-7.76 (m, 1H), 7.72-7.62 (m, 2H), 7.33- |
| | | | | 7.10 (m, 3H), 6.44-6.35 (m, 1H), 4.74 (s, 2H), 4.62- |
| | | | | 4.56 (m, 4H), 4.53-3.82 (m, 2H), 3.80-3.65 (m, 3H), |
| | | | | 3.17 (s, 1H), 3.10-2.78 (m, 4H), 2.70-2.59 (m, 1H), |
| | | | | 2.08 (t, J = 12.0 Hz, 1H), 1.80-1.67 (m, 3H), 1.51- |
| | | | | 1.34 (m, 1H), 1.13-0.92 (m, 2H), 0.75 (s, 2H) |
| I-723 | FK | MR | 604.4 | 12.23-12.12 (m, 1H), 8.62-8.52 (m, 1H), 7.95-7.74 |
| | | | | (m, 2H), 7.41-7.18 (m, 2H), 6.79-6.71 (m, 1H), 6.18- |
| | | | | 6.07 (m, 1H), 4.39-4.30 (m, 2H), 4.01-3.93 (m, |
| | | | | 3H), 3.89-3.79 (m, 7H), 3.67-3.58 (m, 2H), 3.28 (s, |
| | | | | 4H), 2.41-2.26 (m, 2H), 2.14-2.04 (m, 3H) |
| I-724 | OA | DE | 694.2 | 12.26 (d, J = 6.4 Hz, 1H), 8.26 (s, 1H), 8.19 (s, 1H), |
| | | | | 8.15-8.06 (m, 1H), 7.79 (d, J = 4.8 Hz, 1H), 7.72- |
| | | | | 7.64 (m, 1H), 7.26 (d, J = 7.6 Hz, 1H), 7.09-6.98 (m, |
| | | | | 1H), 6.91 (dd, J = 4.8, 7.6 Hz, 1H), 6.53 (s, 1H), 6.21- |
| | | | | 6.11 (m, 1H), 4.62 (q, J = 6.8 Hz, 2H), 4.43-4.29 (m, |
| | | | | 2H), 3.80 (s, 6H), 3.78-3.76 (m, 1H), 3.68-3.58 (m, |
| | | | | 2H), 3.33 (s, 4H), 3.29 (s, 3H), 3.09 (q, J = 6.8 Hz, 2H), |
| | | | | 2.49-2.49 (m, 3H), 2.40-2.25 (m, 2H), 2.23 (s, 3H) |
| I-725 | OF | B | 674.4 | 12.05-12.02 (m, 1H), 8.13-8.09 (m, 1H), 7.81 (d, J = |
| | | | | 2.4 Hz, 1H), 7.73-7.64 (m, 1H), 7.38 (dd, J = 2.4, 10.4 |
| | | | | Hz, 1H), 6.91 (d, J = 1.2 Hz, 1H), 6.82-6.75 (m, 1H), |
| | | | | 6.06 (s, 1H), 4.62 (q, J = 6.4 Hz, 2H), 4.34-4.27 (m, |
| | | | | 2H), 3.86 (s, 3H), 3.83 (d, J = 2.4 Hz, 4H), 3.66-3.56 |
| | | | | (m, 3H), 3.13-3.04 (m, 3H), 2.93-2.85 (m, 4H), 2.36- |
| | | | | 2.31 (m, 2H), 2.28-2.25 (m, 1H), 2.23 (s, 3H), 2.12- |
| | | | | 2.07 (m, 2H), 1.80 (d, J = 3.2 Hz, 4H) |
| I-726 | OT | DQ | 720.4 | 8.33-8.27 (m, 1H), 8.13-8.08 (m, 1H), 7.85-7.77 (m, |
| | | | | 2H), 7.68 (br d, J = 1.6 Hz, 1H), 7.58-7.52 (m, 1H), |
| | | | | 7.38-7.33 (m, 1H), 7.15 (br d, J = 3.6 Hz, 1H), 6.97 |
| | | | | (br s, 1H), 6.79-6.61 (m, 1H), 6.05-5.95 (m, 1H), |
| | | | | 4.63 (br s, 2H), 4.29-4.21 (m, 1H), 4.17-4.11 (m, |
| | | | | 1H), 3.82 (br d, J = 8.4 Hz, 6H), 3.71-3.63 (m, 5H), |
| | | | | 3.62-3.48 (m, 4H), 3.25 (br d, J = 2.0 Hz, 3H), 3.10 |
| | | | | (br d, J = 2.4 Hz, 2H), 2.34-2.25 (m, 2H), 0.58-0.47 |
| | | | | (m, 1H), 0.12-0.04 (m, 2H), −0.22-−0.30 (m, 2H) |
| I-727 | NS | OU | 630.2 | 8.62-8.54 (m, 2H), 8.13-8.09 (m, 1H), 7.91-7.86 (m, |
| | | | | 1H), 7.70-7.67 (m, 1H), 7.45 (d, J = 2.4 Hz, 1H), 7.28- |
| | | | | 7.09 (m, 2H), 6.85-6.65 (m, 1H), 6.06-5.96 (m, |
| | | | | 1H), 4.78-4.71 (m, 2H), 4.66-4.57 (m, 4H), 4.35 (t, J = |
| | | | | 17.6 Hz, 1H), 4.09 (d, J = 17.6 Hz, 1H), 3.80-3.69 |
| | | | | (m, 3H), 3.65-3.51 (m, 2H), 3.51-3.43 (m, 1H), 3.42- |
| | | | | 3.36 (m, 1H), 3.16-3.07 (m, 2H), 2.44-2.38 (m, |
| | | | | 1H), 2.35-2.23 (m, 3H), 1.02 (q, J = 7.2 Hz, 3H), 0.47 |
| | | | | (d, J = 5.6 Hz, 1H), 0.15-0.07 (m, 2H),-0.26 (d, J = |
| | | | | 4.0 Hz, 2H) |
| I-728 | OT | IZ | 730.6 | 8.77 (d, J = 3.2 Hz, 1H), 8.39-8.32 (m, 1H), 8.05- |
| | | | | 8.00 (m, 2H), 7.98-7.92 (m, 2H), 7.61 (dd, J = 2.4, |
| | | | | 10.3 Hz, 1H), 7.52 (dd, J = 4.8, 7.6 Hz, 1H), 7.27 (d, J = |
| | | | | 1.6 Hz, 1H), 7.07-6.89 (m, 1H), 6.30-6.20 (m, 1H), |
| | | | | 4.88 (t, J = 6.4 Hz, 2H), 4.61-4.49 (m, 1H), 4.41-4.34 |
| | | | | (m, 1H), 4.09 (s, 3H), 3.93 (s, 4H), 3.87-3.75 (m, 4H), |
| | | | | 3.51 (s, 4H), 3.38-3.32 (m, 2H), 2.58-2.49 (m, 2H), |
| | | | | 1.92-1.84 (m, 1H), 1.30-1.18 (m, 2H), 1.07-0.97 (m, |
| | | | | 2H), 0.85-0.74 (m, 1H), 0.36-0.29 (m, 2H), 0.06-−0.03 |
| | | | | (m, 2H) |
| I-729 | OT | CG | 720.4 | 8.52 (s, 1H), 8.34 (d, J = 4.4 Hz, 1H), 8.11 (s, 1H), 7.79 |
| | | | | (d, J = 2.4 Hz, 1H), 7.71-7.68 (m, 2H), 7.46-7.43 (m, |
| | | | | 1H), 7.36 (dd, J = 2.4, 10.4 Hz, 1H), 6.98 (d, J = 1.2 |
| | | | | Hz, 1H), 6.79-6.63 (m, 1H), 6.05-5.98 (m, 1H), 4.63 |
| | | | | (t, J = 6.8 Hz, 2H), 4.28 (br t, J = 17.4 Hz, 1H), 4.18- |
| | | | | 4.12 (m, 1H), 3.84 (s, 6H), 3.67 (br d, J = 8.6 Hz, 4H), |
| | | | | 3.62-3.50 (m, 4H), 3.25 (br s, 4H), 3.10 (br t, J = 6.8 |
| | | | | Hz, 2H), 2.33 (br s, 1H), 2.29-2.25 (m, 1H), 0.55- |
| | | | | 0.47 (m, 1H), 0.11-0.04 (m, 2H), −0.26 (br s, 2H) |
| I-730 | OQ | IG | 700.3 | 12.13 (d, J = 2.0 Hz, 1H), 8.27 (s, 1H), 8.14-8.08 (m, |
| | | | | 1H), 7.81 (d, J = 2.4 Hz, 1H), 7.73-7.67 (m, 1H), 7.38 |
| | | | | (dd, J = 2.4, 10.4 Hz, 1H), 6.79-6.66 (m, 1H), 6.51 (s, |
| | | | | 1H), 6.10 (d, J = 7.2 Hz, 1H), 4.63 (q, J = 6.4 Hz, 2H), |
| | | | | 4.35-4.28 (m, 2H), 3.86 (s, 3H), 3.80 (s, 4H), 3.65- |
| | | | | 3.58 (m, 2H), 3.28-3.27 (m, 2H), 3.10 (d, J = 6.4 Hz, |
| | | | | 2H), 3.00 (s, 2H), 2.58 (d, J = 6.4 Hz, 4H), 2.34 (d, J = |
| | | | | 2.0 Hz, 2H), 2.26 (s, 3H), 2.26-2.23 (m, 2H), 1.81 (q, J = |
| | | | | 7.2 Hz, 2H), 0.83 (t, J = 7.6 Hz, 3H) |
| I-731 | OP | IG | 686.4 | 12.28-11.89 (m, 1H), 8.33 (s, 1H), 8.15-8.06 (m, |
| | | | | 1H), 7.80 (d, J = 2.4 Hz, 1H), 7.73-7.65 (m, 1H), 7.38 |
| | | | | (dd, J = 2.4, 10.4 Hz, 1H), 6.74-6.62 (m, 1H), 6.61- |
| | | | | 6.55 (m, 1H), 6.14-6.02 (m, 1H), 4.62 (q, J = 6.4 Hz, |
| | | | | 2H), 4.39-4.24 (m, 2H), 3.85 (s, 3H), 3.82-3.76 (m, |
| | | | | 4H), 3.67-3.57 (m, 2H), 3.39 (br s, 4H), 3.12-3.07 |
| | | | | (m, 2H), 3.03-2.97 (m, 2H), 2.33-2.26 (m, 6H), 1.81- |
| | | | | 1.76 (m, 2H), 0.84-0.79 (m, 3H) |
| I-732 | OT | OV | 718.6 | 8.60 (d, J = 5.2 Hz, 1H), 8.56 (d, J = 3.2 Hz, 1H), 8.12- |
| | | | | 8.09 (m, 1H), 7.78 (d, J = 2.4 Hz, 1H), 7.73-7.68 (m, |
| | | | | 2H), 7.44 (d, J = 5.2 Hz, 1H), 7.36 (dd, J = 2.4, 10.4 |
| | | | | Hz, 1H), 6.96 (d, J = 1.2 Hz, 1H), 6.83-6.67 (m, 1H), |
| | | | | 6.03-5.96 (m, 1H), 4.62 (br t, J = 6.4 Hz, 2H), 4.39- |
| | | | | 4.29 (m, 1H), 4.12-4.05 (m, 1H), 3.83 (s, 3H), 3.71- |
| | | | | 3.38 (m, 10H), 3.24 (br s, 4H), 3.09 (br t, J = 6.8 Hz, |
| | | | | 2H), 2.30 (br s, 1H), 2.25 (br s, 1H), 1.00 (t, J = 7.2 Hz, |
| | | | | 3H), 0.11-0.08 (m, 1H), −0.06 (s, 2H), −0.22-−0.28 |
| | | | | (m, 2H) |
| I-733 | OT | OW | 718.4 | 8.65-8.62 (m, 1H), 8.12-8.10 (m, 1H), 7.84-7.80 (m, |
| | | | | 1H), 7.79 (d, J = 2.4 Hz, 1H), 7.72-7.68 (m, 2H), 7.38- |
| | | | | 7.35 (m, 2H), 6.95 (d, J = 1.6 Hz, 1H), 6.83-6.66 (m, |
| | | | | 1H), 6.04-5.96 (m, 1H), 4.63 (br t, J = 6.8 Hz, 2H), |
| | | | | 4.39-4.30 (m, 1H), 4.09 (br d, J = 16.0 Hz, 1H), 3.84 |
| | | | | (s, 3H), 3.81-3.72 (m, 1H), 3.66 (br d, J = 5.2 Hz, 4H), |
| | | | | 3.62-3.35 (m, 5H), 3.24 (br s, 4H), 3.09 (br t, J = 6.8 |
| | | | | Hz, 2H), 2.33-2.31 (m, 1H), 2.25 (br d, J = 3.2 Hz, |
| | | | | 1H), 1.06 (t, J = 7.6 Hz, 3H), 0.51-0.44 (m, 1H), 0.12- |
| | | | | 0.05 (m, 2H), −0.22-−0.29 (m, 2H) |
| I-734 | RM | M | 693.5 | 8.10 (s, 1H), 7.78 (d, J = 2.4 Hz, 1H), 7.69 (s, 1H), 7.65- |
| | | | | 7.60 (m, 1H), 7.50-7.42 (m, 1H), 7.39-7.31 (m, |
| | | | | 2H), 7.15 (d, J = 8.4 Hz, 1H), 7.07 (t, J = 7.2 Hz, 1H), |
| | | | | 6.89 (s, 1H), 6.70-6.52 (m, 1H), 6.00 (s, 1H), 4.62 (t, J = |
| | | | | 6.8 Hz, 2H), 4.19 (d, J = 8.8 Hz, 2H), 3.84 (s, 3H), |
| | | | | 3.80-3.57 (m, 11H), 3.24 (s, 4H), 3.09 (t, J = 6.8 Hz, |
| | | | | 2H), 2.33 (s, 2H), 0.64 (d, J = 8.8 Hz, 3H) |
| I-735 | PD | IG | 696.4 | 12.44-12.20 (m, 1H), 8.15-8.07 (m, 1H), 7.78 (d, J = |
| | | | | 2.4 Hz, 1H), 7.71-7.65 (m, 1H), 7.53 (dd, J = 3.2, 8.0 |
| | | | | Hz, 1H), 7.36 (dd, J = 2.4, 10.4 Hz, 1H), 7.16 (d, J = |
| | | | | 7.6 Hz, 1H), 6.95-6.80 (m, 1H), 6.35 (s, 1H), 6.22- |
| | | | | 6.10 (m, 1H), 4.61 (q, J = 7.2 Hz, 2H), 4.40-4.27 (m, |
| | | | | 2H), 3.83 (s, 3H), 3.80-3.73 (m, 4H), 3.64 (t, J = 5.6 |
| | | | | Hz, 1H), 3.59 (t, J = 5.6 Hz, 1H), 3.26-3.21 (m, 3H), |
| | | | | 3.09 (q, J = 6.8 Hz, 2H), 2.61-2.54 (m, 3H), 2.21- |
| | | | | 2.51 (m, 3H), 2.40-2.24 (m, 2H), 1.05 (t, J = 7.6 Hz, 3H) |
| I-736 | NS | M | 631.4 | 8.13-8.08 (m, 1H), 7.81 (d, J = 6.4 Hz, 1H), 7.69 (s, |
| | | | | 1H), 7.46 (t, J = 8.0 Hz, 1H), 7.35 (d, J = 6.4 Hz, 1H), |
| | | | | 7.26 (s, 1H), 7.29-7.12 (m, 1H), 7.11-7.03 (m, 2H), |
| | | | | 6.60 (s, 1H), 5.98 (s, 1H), 4.75 (d, J = 10.8 Hz, 2H), |
| | | | | 4.66-4.57 (m, 4H), 4.28 (t, J = 18.4 Hz, 1H), 4.19- |
| | | | | 4.09 (m, 1H), 3.79 (s, 1H), 3.71 (s, 5H), 3.67-3.48 (m, |
| | | | | 4H), 3.14-3.04 (m, 2H), 2.34-2.24 (m, 2H), 0.55 (dd, |
| | | | | J = 6.0, 9.6 Hz, 1H), 0.12-0.00 (m, 2H), −0.29 (d, J = |
| | | | | 3.6 Hz, 2H) |
| I-737 | PD | PC | 710.4 | 12.43-12.15 (m, 1H), 8.27 (s, 1H), 8.16-8.06 (m, |
| | | | | 1H), 7.85-7.50 (m, 1H), 7.31-7.27 (m, 2H), 6.94- |
| | | | | 6.83 (m, 1H), 6.35 (s, 1H), 6.19-6.13 (m, 1H), 4.62 (q, |
| | | | | J = 7.0 Hz, 2H), 4.39-4.31 (m, 2H), 3.79 (s, 3H), 3.75 |
| | | | | (s, 4H), 3.64 (t, J = 5.3 Hz, 1H), 3.59 (t, J = 5.8 Hz, |
| | | | | 1H), 3.35 (s, 2H), 3.22 (s, 4H), 3.12-3.07 (m, 2H), |
| | | | | 2.48-2.43 (m, 2H), 2.42-2.31 (m, 2H), 2.31-2.26 (m, |
| | | | | 1H), 2.24 (d, J = 2.8 Hz, 3H), 0.99 (t, J = 7.6 Hz, 3H) |
| I-738 | PD | RO | 709.4 | 12.33-12.21 (m, 1H), 8.27 (s, 1H), 7.73-7.68 (m, |
| | | | | 1H), 7.45-7.33 (m, 1H), 7.31-7.26 (m, 2H), 6.92- |
| | | | | 6.83 (m, 1H), 6.35 (s, 1H), 6.21-6.13 (m, 2H), 4.40- |
| | | | | 4.25 (m, 5H), 3.79 (s, 3H), 3.75 (s, 4H), 3.64 (t, J = 5.6 |
| | | | | Hz, 1H), 3.57 (t, J = 5.6 Hz, 1H), 3.22 (s, 4H), 2.97 (q, |
| | | | | J = 7.2 Hz, 2H), 2.45 (s, 4H), 2.33 (s, 1H), 2.24 (d, J = |
| | | | | 2.8 Hz, 4H), 0.99 (t, J = 7.6 Hz, 3H) |
| I-739 | PD | RQ | 695.5 | 12.30 (s, 1H), 8.27 (s, 1H), 8.17-8.05 (m, 1H), 7.76- |
| | | | | 7.61 (m, 1H), 7.28 (s, 1H), 6.94-6.82 (m, 3H), 6.67 |
| | | | | (dt, J = 3.2, 8.4 Hz, 1H), 6.34 (s, 1H), 6.22-6.09 (m, |
| | | | | 1H), 4.62 (q, J = 7.2 Hz, 2H), 4.46-4.26 (m, 2H), 3.87- |
| | | | | 3.72 (m, 7H), 3.70-3.57 (m, 2H), 3.09 (q, J = 6.0 Hz, |
| | | | | 2H), 2.91 (s, 4H), 2.69-2.60 (m, 1H), 2.48-2.42 (m, |
| | | | | 3H), 2.40-2.20 (m, 3H), 0.99 (t, J = 7.6 Hz, 3H) |
| I-740 | PF | PC | 724.2 | 12.31 (s, 1H), 8.24 (s, 1H), 8.13-8.07 (m, 1H), 7.73- |
| | | | | 7.63 (m, 1H), 7.35 (s, 1H), 7.29 (d, J = 10.8 Hz, 1H), |
| | | | | 6.92-6.81 (m, 1H), 6.33 (d, J = 2.4 Hz, 1H), 6.20- |
| | | | | 6.12 (m, 1H), 4.64-4.59 (m, 2H), 4.40-4.31 (m, 2H), |
| | | | | 3.79 (s, 3H), 3.76 (s, 4H), 3.64 (t, J = 5.6 Hz, 1H), |
| | | | | 3.59 (t, J = 5.2 Hz, 2H), 3.22 (s, 4H), 3.11-3.06 (m, |
| | | | | 2H), 2.86-2.80 (m, 1H), 2.52 (s, 2H), 2.34 (d, J = 2.0 |
| | | | | Hz, 2H), 2.24 (d, J = 2.8 Hz, 3H), 1.10 (s, 3H), 1.05 (s, 3H) |
| I-741 | RP | RO | 723.5 | 12.29 (s, 1H), 8.31 (s, 1H), 7.76-7.66 (m, 1H), 7.45- |
| | | | | 7.34 (m, 1H), 7.31-7.27 (m, 2H), 6.93-6.84 (m, 1H), |
| | | | | 6.35 (s, 1H), 6.21-6.13 (m, 2H), 4.38-4.34 (m, 3H), |
| | | | | 4.33-4.28 (m, 1H), 3.79 (s, 3H), 3.76 (s, 4H), 3.63 (t, J = |
| | | | | 5.6 Hz, 1H), 3.59-3.55 (m, 1H), 3.22 (s, 4H), 3.00- |
| | | | | 2.95 (m, 2H), 2.80 (q, J = 7.6 Hz, 2H), 2.48-2.44 (m, |
| | | | | 2H), 2.33 (s, 2H), 2.24 (d, J = 2.4 Hz, 3H), 1.29 (t, J = |
| | | | | 7.6 Hz, 3H), 1.00 (t, J = 7.6 Hz, 3H) |
| I-742 | PD | IG | 696.4 | 12.30 (s, 1H), 8.40-8.20 (m, 1H), 8.17-8.04 (m, 1H), |
| | | | | 7.77 (s, 1H), 7.72-7.61 (m, 1H), 7.44-7.32 (m, 1H), |
| | | | | 7.28 (s, 1H), 6.97-6.80 (m, 1H), 6.34 (s, 1H), 6.22- |
| | | | | 6.09 (m, 1H), 4.74-4.54 (m, 2H), 4.44-4.25 (m, 2H), |
| | | | | 3.96-3.69 (m, 7H), 3.65-3.55 (m, 2H), 3.29-3.17 (m, |
| | | | | 6H), 3.14-3.02 (m, 2H), 2.47 (s, 3H), 2.40-2.21 (m, |
| | | | | 2H), 1.15-0.92 (m, 3H) |
| I-743 | RB | IG | 686.4 | 12.11 (s, 1H), 8.38 (s, 1H), 8.15-8.08 (m, 1H), 7.81 (d, |
| | | | | J = 2.4 Hz, 1H), 7.73-7.65 (m, 1H), 7.38 (dd, J = 2.4, |
| | | | | 10.4 Hz, 1H), 6.73-6.59 (m, 2H), 6.15-6.05 (m, 1H), |
| | | | | 4.68-4.60 (m, 2H), 4.38-4.28 (m, 2H), 3.86 (s, 3H), |
| | | | | 3.84-3.79 (m, 4H), 3.69-3.52 (m, 4H), 3.17-3.04 (m, |
| | | | | 4H), 2.98 (d, J = 6.4 Hz, 2H), 2.34-2.26 (m, 2H), 2.13 |
| | | | | (d, J = 1.2 Hz, 2H), 1.80 (q, J = 7.2 Hz, 2H), 1.34-1.18 |
| | | | | (m, 2H), 0.86-0.82 (m, 3H) |
| I-744 | IC | QA | 671.4 | 12.30 (d, J = 6.0 Hz, 1H), 8.17-8.03 (m, 1H), 7.78 (d, J = |
| | | | | 2.4 Hz, 1H), 7.72-7.65 (m, 1H), 7.64-7.55 (m, 1H), |
| | | | | 7.52-7.43 (m, 1H), 7.41-7.29 (m, 3H), 7.10-6.95 (m, |
| | | | | 1H), 6.57 (s, 1H), 6.24-6.09 (m, 1H), 4.70-4.53 (m, |
| | | | | 2H), 4.41-4.27 (m, 2H), 3.83 (s, 3H), 3.79 (d, J = 2.4 |
| | | | | Hz, 4H), 3.69-3.56 (m, 2H), 3.25 (s, 4H), 3.14-3.05 |
| | | | | (m, 2H), 2.42-2.21 (m, 2H) |
| I-745 | IC | FZ | 678.3 | 12.39 (br s, 1H), 8.19-8.06 (m, 1H), 8.04-7.94 (m, |
| | | | | 1H), 7.90-7.50 (m, 5H), 7.36 (dd, J = 2.4, 10.0 Hz, |
| | | | | 1H), 7.22-7.04 (m, 1H), 6.62 (d, J = 2.8 Hz, 1H), 6.28- |
| | | | | 6.10 (m, 1H), 4.75-4.53 (m, 2H), 4.47-4.27 (m, |
| | | | | 2H), 3.84 (s, 3H), 3.80 (br s, 4H), 3.69-3.56 (m, 2H), |
| | | | | 3.27 (br d, J = 14.0 Hz, 4H), 3.14-3.03 (m, 2H), 2.38- |
| | | | | 2.26 (m, 2H) |
| I-746 | IC | QB | 689.3 | 12.34 (s, 1H), 8.22-8.00 (m, 1H), 7.78 (d, J = 2.4 Hz, |
| | | | | 1H), 7.72-7.64 (m, 1H), 7.51-7.25 (m, 4H), 7.14- |
| | | | | 6.99 (m, 1H), 6.62 (d, J = 2.0 Hz, 1H), 6.25-6.07 (m, |
| | | | | 1H), 4.62 (q, J = 7.2 Hz, 2H), 4.47-4.26 (m, 2H), 3.84 |
| | | | | (s, 3H), 3.79 (s, 4H), 3.69-3.55 (m, 2H), 3.25 (s, 4H), |
| | | | | 3.15-3.05 (m, 2H), 2.39-2.21 (m, 2H) |
| I-747 | QN | QB | 615.3 | 12.51-12.03 (m, 1H), 8.15-8.07 (m, 1H), 7.72-7.65 |
| | | | | (m, 1H), 7.50-7.40 (m, 2H), 7.37-7.24 (m, 2H), 7.14- |
| | | | | 7.03 (m, 1H), 6.93 (s, 1H), 6.18 (d, J = 8.0 Hz, 1H), |
| | | | | 5.04 (s, 1H), 4.83 (d, J = 16.4 Hz, 2H), 4.62 (q, J = 7.2 |
| | | | | Hz, 3H), 4.40-4.31 (m, 2H), 4.14-4.01 (m, 2H), 3.68- |
| | | | | 3.59 (m, 2H), 3.14-3.07 (m, 2H), 2.36 (d, J = 2.0 Hz, |
| | | | | 1H), 2.28 (d, J = 1.6 Hz, 1H), 1.38-1.29 (m, 3H) |
| I-748 | QN | FZ | 604.3 | 12.43 (s, 1H), 8.15-8.08 (m, 1H), 8.01 (d, J = 7.6 Hz, |
| | | | | 1H), 7.88-7.81 (m, 1H), 7.81-7.74 (m, 1H), 7.71- |
| | | | | 7.59 (m, 2H), 7.33-7.24 (m, 1H), 7.21-7.10 (m, 1H), |
| | | | | 6.95 (dd, J = 2.1, 6.8 Hz, 1H), 6.18 (s, 1H), 5.04 (s, |
| | | | | 1H), 4.83 (d, J = 14.0 Hz, 2H), 4.67-4.58 (m, 3H), |
| | | | | 4.42-4.31 (m, 2H), 4.15-4.01 (m, 2H), 3.68-3.57 (m, |
| | | | | 2H), 3.14-3.07 (m, 2H), 2.37 (d, J = 1.2 Hz, 1H), 2.29 |
| | | | | (d, J = 2.4 Hz, 1H), 1.39-1.29 (m, 3H) |
| I-749 | SQ | QD | 605.4 | 12.59-12.42 (m, 1H), 9.08-8.97 (m, 1H), 8.89 (d, J = |
| | | | | 5.2 Hz, 1H), 8.16-8.07 (m, 1H), 8.07-8.03 (m, 1H), |
| | | | | 7.72-7.64 (m, 1H), 7.32-7.18 (m, 1H), 7.06 (s, 1H), |
| | | | | 6.26-6.17 (m, 1H), 5.10-4.99 (m, 1H), 4.88-4.82 (m, |
| | | | | 2H), 4.68-4.56 (m, 3H), 4.43-4.30 (m, 2H), 4.15- |
| | | | | 3.98 (m, 2H), 3.68-3.56 (m, 2H), 3.13-3.03 (m, 2H), |
| | | | | 2.37 (s, 1H), 2.30 (d, J = 2.0 Hz, 1H), 1.40-1.27 (m, 3H) |
| I-750 | SQ | IG | 679.4 | 12.64-12.28 (m, 1H), 9.01 (s, 1H), 8.86 (d, J = 5.2 Hz, |
| | | | | 1H), 8.16-8.07 (m, 1H), 8.03 (d, J = 4.8 Hz, 1H), 7.79 |
| | | | | (d, J = 2.4 Hz, 1H), 7.72-7.64 (m, 1H), 7.37 (dd, J = |
| | | | | 2.4, 10.4 Hz, 1H), 7.31-7.15 (m, 1H), 6.74 (s, 1H), |
| | | | | 6.30-6.14 (m, 1H), 4.62 (q, J = 6.8 Hz, 2H), 4.44- |
| | | | | 4.29 (m, 2H), 3.84 (s, 3H), 3.81 (s, 4H), 3.68-3.59 (m, |
| | | | | 2H), 3.29-3.21 (m, 4H), 3.13-3.04 (m, 2H), 2.38- |
| | | | | 2.27 (m, 2H) |
| I-751 | SR | QD | 581.3 | 9.25 (s, 1H), 8.35-8.23 (m, 1H), 8.18-8.05 (m, 1H), |
| | | | | 7.82 (d, J = 4.4 Hz, 1H), 7.69 (d, J = 11.6 Hz, 1H), 7.63- |
| | | | | 7.53 (m, 2H), 7.31 (s, 1H), 6.23 (s, 1H), 5.09 (s, 1H), |
| | | | | 4.93 (s, 1H), 4.85 (s, 1H), 4.68-4.59 (m, 3H), 4.44- |
| | | | | 4.36 (m, 2H), 4.16-4.03 (m, 2H), 3.70-3.60 (m, 2H), |
| | | | | 3.11 (d, J = 7.2 Hz, 2H), 2.38 (d, J = 1.6 Hz, 1H), 2.33- |
| | | | | 2.29 (m, 1H), 1.40-1.31 (m, 3H) |
| I-752 | QC | QD | 583.4 | 12.45-12.35 (m, 1H), 8.11 (d, J = 11.2 Hz, 1H), 7.71- |
| | | | | 7.65 (m, 1H), 7.57 (s, 1H), 7.32-7.24 (m, 1H), 7.14- |
| | | | | 7.05 (m, 1H), 6.97-6.92 (m, 1H), 6.53 (s, 1H), 6.22- |
| | | | | 6.15 (m, 1H), 5.08 (s, 1H), 4.89 (s, 1H), 4.82 (s, 1H), |
| | | | | 4.65-4.59 (m, 3H), 4.40-4.32 (m, 2H), 4.15-4.05 (m, |
| | | | | 2H), 3.84-3.80 (m, 3H), 3.67-3.59 (m, 2H), 3.12- |
| | | | | 3.08 (m, 2H), 2.36 (d, J = 1.2 Hz, 1H), 2.31-2.26 (m, |
| | | | | 1H), 1.38-1.31 (m, 3H) |
| I-753 | SS | QD | 583.3 | 12.40-12.15 (m, 1H), 8.16-8.05 (m, 1H), 7.74-7.64 |
| | | | | (m, 1H), 7.36-7.23 (m, 2H), 7.22-7.12 (m, 2H), 7.11- |
| | | | | 7.05 (m, 1H), 6.26-6.11 (m, 1H), 5.06 (s, 1H), 4.85 |
| | | | | (d, J = 19.6 Hz, 2H), 4.67-4.56 (m, 3H), 4.41-4.33 |
| | | | | (m, 2H), 4.15-4.05 (m, 2H), 3.75-3.69 (m, 3H), 3.67 |
| | | | | (t, J = 5.6 Hz, 1H), 3.61 (t, J = 5.6 Hz, 1H), 3.11 (q, J = |
| | | | | 6.4 Hz, 2H), 2.37 (s, 1H), 2.29 (d, J = 3.6 Hz, 1H), 1.39- |
| | | | | 1.30 (m, 3H) |
| I-754 | ST | QD | 622.2 | 12.39-12.22 (m, 1H), 8.42-8.30 (m, 1H), 8.13-8.05 |
| | | | | (m, 1H), 7.71-7.62 (m, 1H), 7.31-7.22 (m, 2H), 6.98- |
| | | | | 6.85 (m, 1H), 6.71 (s, 1H), 6.21-6.14 (m, 1H), 4.82 |
| | | | | (s, 2H), 4.66-4.59 (m, 3H), 4.40-4.32 (m, 2H), 4.14- |
| | | | | 4.02 (m, 2H), 3.67-3.58 (m, 2H), 3.12-3.07 (m, 2H), |
| | | | | 2.79 (q, J = 7.6 Hz, 2H), 2.33 (dd, J = 2.4, 4.4 Hz, 2H), |
| | | | | 2.27 (s, 1H), 2.20-2.16 (m, 3H), 1.35 (t, J = 7.2 Hz, |
| | | | | 2H), 1.30 (t, J = 7.6 Hz, 4H) |
| I-755 | QL | QD | 586.2 | 12.48 (d, J = 3.6 Hz, 1H), 8.15-8.08 (m, 1H), 8.05- |
| | | | | 8.00 (m, 1H), 7.85 (d, J = 3.2 Hz, 1H), 7.76-7.66 (m, |
| | | | | 2H), 7.64-7.57 (m, 1H), 7.32 (s, 1H), 6.18 (s, 1H), |
| | | | | 5.13 (s, 1H), 4.97 (s, 1H), 4.85 (s, 1H), 4.67-4.59 (m, |
| | | | | 3H), 4.41-4.33 (m, 2H), 4.17-4.07 (m, 2H), 3.69- |
| | | | | 3.60 (m, 2H), 3.14-3.07 (m, 2H), 2.40-2.28 (m, 2H), |
| | | | | 1.40-1.33 (m, 3H) |
| I-756 | QN | QM | 597.3 | 12.40 (s, 1H), 8.19-8.00 (m, 1H), 7.76-7.64 (m, 1H), |
| | | | | 7.60 (s, 1H), 7.33-7.24 (m, 1H), 7.10-6.97 (m, 1H), |
| | | | | 6.91-6.83 (m, 1H), 6.48 (s, 1H), 6.23-6.12 (m, 1H), |
| | | | | 5.16-4.77 (m, 3H), 4.62 (q, J = 7.2 Hz, 3H), 4.41- |
| | | | | 4.30 (m, 2H), 4.17-4.02 (m, 4H), 3.74-3.53 (m, 2H), |
| | | | | 3.10 (q, J = 6.4 Hz, 2H), 2.37-2.26 (m, 2H), 1.40- |
| | | | | 1.23 (m, 6H) |
| I-757 | QF | PC | 710.4 | 12.28 (s, 1H), 8.34 (s, 1H), 8.12-8.08 (m, 1H), 7.71- |
| | | | | 7.65 (m, 1H), 7.29 (d, J = 10.8 Hz, 1H), 7.25 (s, 1H), |
| | | | | 6.96-6.86 (m, 1H), 6.40 (s, 1H), 6.20-6.14 (m, 1H), |
| | | | | 4.62 (d, J = 7.2 Hz, 2H), 4.39-4.32 (m, 2H), 3.79 (s, |
| | | | | 3H), 3.76 (s, 4H), 3.65 (t, J = 6.0 Hz, 1H), 3.61-3.58 |
| | | | | (m, 1H), 3.23 (s, 4H), 3.09 (d, J = 4.0 Hz, 2H), 2.78 |
| | | | | (d, J = 7.6 Hz, 2H), 2.38-2.31 (m, 2H), 2.24 (d, J = 2.8 |
| | | | | Hz, 3H), 2.15 (s, 3H), 1.28 (t, J = 7.6 Hz, 3H) |
| I-758 | SP | QG | 613.3 | 12.23-12.06 (m, 1H), 8.25 (s, 1H), 8.14-8.13 (m, |
| | | | | 1H), 8.12 (s, 1H), 7.73-7.61 (m, 1H), 7.10-7.05 (m, |
| | | | | 1H), 6.99-6.88 (m, 1H), 6.44-6.32 (m, 1H), 6.19- |
| | | | | 6.06 (m, 1H), 4.69-4.58 (m, 2H), 4.38-4.28 (m, 2H), |
| | | | | 3.86-3.70 (m, 4H), 3.67-3.56 (m, 7H), 3.13-3.03 (m, |
| | | | | 3H), 2.55 (s, 4H), 2.37-2.25 (m, 2H), 1.68-1.59 (m, |
| | | | | 1H), 0.48-0.27 (m, 4H) |
| I-759 | QF | QG | 611.3 | 12.34-12.20 (m, 1H), 8.33 (s, 1H), 8.14-8.06 (m, |
| | | | | 1H), 7.74-7.61 (m, 1H), 7.25 (s, 1H), 7.00-6.83 (m, |
| | | | | 1H), 6.32 (d, J = 2.0 Hz, 1H), 6.23-6.13 (m, 1H), 4.62 |
| | | | | (q, J = 7.2 Hz, 2H), 4.39-4.31 (m, 2H), 3.64 (br d, J = |
| | | | | 6.0 Hz, 1H), 3.59 (br s, 5H), 3.12-3.07 (m, 2H), 2.78 |
| | | | | (q, J = 7.6 Hz, 2H), 2.53 (br s, 3H), 2.38-2.30 (m, 2H), |
| | | | | 2.27 (br d, J = 2.0 Hz, 1H), 2.14 (s, 3H), 1.67-1.60 (m, |
| | | | | 1H), 1.28 (t, J = 7.6 Hz, 3H), 0.44-0.39 (m, 2H), 0.32 |
| | | | | (br d, J = 3.2 Hz, 2H) |
| I-760 | QH | PC | 710.2 | 12.27 (s, 1H), 8.14-8.06 (m, 1H), 7.71-7.66 (m, 1H), |
| | | | | 7.62-7.58 (m, 1H), 7.29 (d, J = 10.8 Hz, 1H), 7.18 (d, J = |
| | | | | 8.0 Hz, 1H), 6.97-6.86 (m, 1H), 6.40 (s, 1H), 6.16 (d, |
| | | | | J = 12.4 Hz, 1H), 4.62 (d, J = 7.2 Hz, 2H), 4.38- |
| | | | | 4.31 (m, 2H), 3.79 (s, 3H), 3.77 (d, J = 2.4 Hz, 4H), |
| | | | | 3.64 (t, J = 5.2 Hz, 1H), 3.59 (s, 1H), 3.23 (s, 4H), |
| | | | | 3.09 (d, J = 6.0 Hz, 2H), 2.78 (d, J = 7.6 Hz, 2H), 2.36 |
| | | | | (s, 2H), 2.32 (d, J = 2.8 Hz, 3H), 2.25 (d, J = 2.8 Hz, |
| | | | | 3H), 1.28 (t, J = 7.6 Hz, 3H) |
| I-761 | QH | QD | 622.4 | 12.29 (br s, 1H), 8.12-8.08 (m, 1H), 7.70-7.61 (m, |
| | | | | 2H), 7.30-7.23 (m, 1H), 7.20 (d, J = 7.6 Hz, 1H), 6.97- |
| | | | | 6.87 (m, 1H), 6.74-6.70 (m, 1H), 6.20-6.15 (m, |
| | | | | 1H), 5.01 (br s, 1H), 4.81 (br d, J = 8.4 Hz, 3H), 4.65- |
| | | | | 4.59 (m, 3H), 4.39-4.32 (m, 2H), 4.13-4.02 (m, 2H), |
| | | | | 3.66-3.58 (m, 2H), 3.09 (br d, J = 5.2 Hz, 2H), 2.80 (d, |
| | | | | J = 7.6 Hz, 2H), 2.36-2.32 (m, 5H), 1.33-1.27 (m, 5H) |
| I-762 | QI | QD | 634.3 | 12.55-11.96 (m, 1H), 8.15-8.06 (m, 1H), 7.74-7.64 |
| | | | | (m, 1H), 7.61-7.51 (m, 1H), 7.32-7.23 (m, 1H), 7.19 |
| | | | | (d, J = 7.6 Hz, 1H), 6.97-6.84 (m, 1H), 6.78-6.64 (m, |
| | | | | 1H), 6.23-6.11 (m, 1H), 5.01 (br s, 1H), 4.83-4.79 |
| | | | | (m, 2H), 4.68-4.55 (m, 3H), 4.42-4.28 (m, 2H), 4.16- |
| | | | | 3.99 (m, 2H), 3.68-3.54 (m, 2H), 3.09 (q, J = 6.8 Hz, |
| | | | | 2H), 2.35-2.26 (m, 3H), 2.18-2.09 (m, 1H), 1.39- |
| | | | | 1.27 (m, 3H), 0.97 (br d, J = 7.2 Hz, 4H) |
| I-763 | QJ | PC | 746.4 | 12.41-12.23 (m, 1H), 8.53 (s, 1H), 8.16-8.03 (m, |
| | | | | 1H), 7.73 (s, 1H), 7.71-7.64 (m, 1H), 7.28 (d, J = 10.4 |
| | | | | Hz, 1H), 7.16-6.84 (m, 2H), 6.41 (s, 1H), 6.24-6.12 |
| | | | | (m, 1H), 4.62 (q, J = 6.4 Hz, 2H), 4.41-4.31 (m, 2H), |
| | | | | 3.79 (s, 3H), 3.78-3.69 (m, 4H), 3.66-3.57 (m, 2H), |
| | | | | 3.23 (s, 4H), 3.12-3.07 (m, 2H), 2.61-2.56 (m, 2H), |
| | | | | 2.35 (s, 2H), 2.24 (d, J = 2.4 Hz, 3H), 1.03 (t, J = 7.2 |
| | | | | Hz, 3H) |
| I-764 | QK | QD | 623.3 | 12.36 (d, J = 2.0 Hz, 1H), 8.49 (d, J = 2.8 Hz, 1H), 8.15- |
| | | | | 8.05 (m, 1H), 7.74-7.61 (m, 1H), 7.33-7.19 (m, |
| | | | | 1H), 7.03-6.88 (m, 1H), 6.79 (s, 1H), 6.18 (d, J = 14.0 |
| | | | | Hz, 1H), 5.10-4.94 (m, 1H), 4.84-4.78 (m, 2H), 4.66- |
| | | | | 4.58 (m, 3H), 4.41-4.30 (m, 2H), 4.11 (d, J = 7.2 Hz, |
| | | | | 2H), 3.67-3.56 (m, 2H), 3.15-3.02 (m, 2H), 2.70- |
| | | | | 2.67 (m, 3H), 2.64-2.59 (m, 2H), 2.34 (d, J = 6.0 Hz, |
| | | | | 2H), 1.39-1.28 (m, 3H), 1.16-1.05 (m, 3H) |
| I-765 | TL | TI | 619.3 | 8.12 (s, 1H), 7.74-7.65 (m, 2H), 7.30-7.14 (m, 1H), |
| | | | | 7.02 (s, 1H), 6.58 (s, 1H), 6.07 (s, 1H), 5.82 (s, 1H), |
| | | | | 4.78-4.72 (m, 2H), 4.64 (t, J = 7.2 Hz, 2H), 4.56 (d, J = |
| | | | | 13.6 Hz, 2H), 4.29-4.18 (m, 2H), 4.14-4.07 (m, |
| | | | | 2H), 3.99 (d, J = 6.4 Hz, 1H), 3.61 (d, J = 4.4 Hz, 1H), |
| | | | | 3.15-3.08 (m, 2H), 2.37-2.29 (m, 5H), 2.21 (s, 2H), |
| | | | | 1.82-1.67 (m, 4H), 1.36 (s, 1H), 1.33-1.16 (m, 3H), |
| | | | | 0.86-0.74 (m, 1H), 0.31-0.21 (m, 2H), 0.07-0.00 (m, 2H) |
| I-766 | QE | QD | 634.3 | 12.42-12.16 (m, 1H), 8.36-8.24 (m, 1H), 8.15-8.06 |
| | | | | (m, 1H), 7.68 (d, J = 10.8 Hz, 1H), 7.35-7.19 (m, 2H), |
| | | | | 6.99-6.81 (m, 1H), 6.76-6.66 (m, 1H), 6.25-6.11 (m, |
| | | | | 1H), 5.08-4.95 (m, 1H), 4.80 (d, J = 6.8 Hz, 2H), 4.62 |
| | | | | (d, J = 7.6 Hz, 3H), 4.42-4.30 (m, 2H), 4.16-3.99 (m, |
| | | | | 2H), 3.68-3.54 (m, 2H), 3.10 (d, J = 0.8 Hz, 2H), 2.35 |
| | | | | (s, 2H), 2.15 (d, J = 7.2 Hz, 3H), 2.12 (s, 1H), 1.40- |
| | | | | 1.28 (m, 3H), 0.97 (d, J = 6.4 Hz, 4H) |
| I-767 | QJ | QD | 658.4 | 12.36 (s, 1H), 8.59-8.53 (m, 1H), 8.14-8.07 (m, 1H), |
| | | | | 7.75 (s, 1H), 7.71-7.65 (m, 1H), 7.30-7.14 (m, 1H), |
| | | | | 7.03 (s, 1H), 6.98-6.87 (m, 1H), 6.72 (s, 1H), 6.24- |
| | | | | 6.15 (m, 1H), 5.00 (s, 1H), 4.79 (s, 2H), 4.68-4.55 (m, |
| | | | | 3H), 4.43-4.28 (m, 2H), 4.15-3.99 (m, 2H), 3.68- |
| | | | | 3.57 (m, 2H), 3.09 (d, J = 6.0 Hz, 2H), 2.60 (d, J = 1.4 |
| | | | | Hz, 2H), 2.38-2.24 (m, 3H), 1.40-1.25 (m, 3H), 1.06 |
| | | | | (td, J = 7.6, 15.2 Hz, 3H) |
| I-551 | TL | TM | 579.4 | 8.34 (s, 1H), 8.12 (s, 1H), 7.72-7.61 (m, 2H), 7.31- |
| | | | | 7.13 (m, 1H), 7.09 (s, 1H), 6.54 (s, 1H), 6.13-6.01 (m, |
| | | | | 1H), 4.78-4.69 (m, 2H), 4.67-4.62 (m, 2H), 4.59- |
| | | | | 4.47 (m, 4H), 4.24 (d, J = 8.0 Hz, 2H), 4.13-4.06 (m, |
| | | | | 1H), 3.98 (d, J = 6.4 Hz, 1H), 3.68-3.59 (m, 2H), 3.15- |
| | | | | 3.10 (m, 2H), 2.43-2.35 (m, 2H), 2.30 (d, J = 3.6 Hz, |
| | | | | 1H), 1.37-1.34 (m, 1H), 1.24 (t, J = 6.4 Hz, 2H), 1.08- |
| | | | | 0.93 (m, 3H), 0.89-0.83 (m, 2H), 0.40-0.31 (m, 2H), |
| | | | | 0.22-0.11 (m, 2H) |

^{a}The coupling was run at 80° C. for 1-6 hr. Final products purified under standard conditions including reverse phase HPLC and prep-TLC under a variety of solvent conditions. Boronic acid or ester could be employed for the coupling. Other catalysts, such as Pd(dppf)Cl₂, and bases, such as K₂CO₃ or Na₂CO₃ could be employed in the coupling.
^{b}The product of the coupling was further deprotected with TFA in DCM at rt for 2 hr.
^{c}Pd(dppf)Cl₂ used as the catalyst in place of Xphos for the coupling.
^{d}Bromide was used in place of chloride for the coupling.
^{e}Iodide was used in place of chloride for the coupling.
^{f} Pd(PPh₃)₄ with Cs₂CO₃ used as catalyst and base. OTf used in place of chloride.
^{g} Cu(OAc)₂ and pyridine used for the coupling in DMF which was run at 60° C. for 5 hrs.
^{h} The product of the coupling was further deprotected with HCl in DCM at rt for 1 hr.
ⁱ The product of the coupling was further deprotected with TBAF in THF at rt for 2 hr. Then the final compound was purified by prep-TLC.
^{j} The product of the coupling was further deprotected with TFA in DCM at rt for 1 hr.

TABLE 9
Compounds synthesized via Method 2, coupling of the corresponding halogens and boronic acids

| | LCMS | |
|---|---|---|
| | (ESI+) m/z | |
| I-# | (M + H)⁺ | ¹H NMR (400 MHz, DMSO-d₆) δ |
| I-1048 | 477.1 | 8.36 (s, 1H), 8.12 (s, 1H), 7.70 (d, J = 2.8 Hz, 1H), 7.62 (br d, J = 20.0 Hz, |
| | | 1H), 7.44-7.21 (m, 2H), 6.36-6.22 (m, 1H), 4.63 (t, J = 6.8 Hz, 2H), 4.37 |
| | | (br d, J = 11.6 Hz, 2H), 3.62 (br s, 2H), 3.57 (br s, 2H), 3.24 (br d, J = 12.0 |
| | | Hz, 6H), 3.16 (br d, J = 6.4 Hz, 1H), 3.10 (br s, 1H), 3.04 (br s, 2H), 2.88 (br |
| | | d, J = 10.0 Hz, 2H), 2.33 (br s, 2H), 1.92 (br s, 4H) |
| I-1049 | 518.2 | 9.07 (d, J = 3.0 Hz, 1H), 8.28 (s, 1H), 8.12 (d, J = 3.8 Hz, 1H), 7.99 (dd, J = |
| | | 1.5, 11.5 Hz, 1H), 7.83 (dd, J = 1.4, 19.1 Hz, 1H), 7.69 (d, J = 3.8 Hz, 1H), |
| | | 7.55 (d, J = 9.5 Hz, 1H), 6.49-6.39 (m, 1H), 4.64 (t, J = 6.8 Hz, 2H), 4.43 (br |
| | | d, J = 10.5 Hz, 2H), 3.83 (s, 3H), 3.66-3.55 (m, 2H), 3.30 (br s, 3H), 3.17- |
| | | 3.11 (m, 2H), 3.05 (br s, 3H), 2.37-2.34 (m, 1H), 2.30-2.24 (m, 1H) |
| I-1050 | 532.5 | 11.72-11.49 (m, 1H), 8.16-8.09 (m, 1H), 7.74-7.62 (m, 1H), 7.44-7.34 |
| | | (m, 1H), 7.25-7.16 (m, 1H), 7.12-7.02 (m, 1H), 6.75-6.65 (m, 1H), 6.61 |
| | | (br d, J = 9.6 Hz, 1H), 6.47 (br s, 1H), 6.35-6.24 (m, 1H), 6.10 (br d, J = 5.5 |
| | | Hz, 1H), 4.69-4.58 (m, 2H), 4.44-4.33 (m, 2H), 3.69-3.53 (m, 2H), 3.21- |
| | | 3.01 (m, 7H), 2.73 (br d, J = 4.8 Hz, 3H), 2.41-2.30 (m, 2H), 2.29-2.19 (m, |
| | | 1H) |
| I-1051 | 528.3 | 11.57-11.37 (m, 1H), 8.16-8.00 (m, 1H), 7.72-7.59 (m, 1H), 7.34-7.24 |
| | | (m, 1H), 7.08 (d, J = 8.0 Hz, 1H), 7.05-6.99 (m, 1H), 6.50 (d, J = 1.6 Hz, |
| | | 1H), 6.30 (s, 1H), 6.22 (d, J = 6.4 Hz, 2H), 5.81-5.85 (m, 1H), 4.69-4.59 |
| | | (m, 2H), 4.45-4.28 (m, 2H), 3.70-3.65 (m, 3H), 3.65-3.54 (m, 3H), 3.20- |
| | | 3.05 (m, 6H), 2.74 (d, J = 4.8 Hz, 3H), 2.37-2.21 (m, 3H) |
| I-1052 | 412.2 | 8.11 (d, J = 7.6 Hz, 1H), 7.69 (d, J = 8.4 Hz, 1H), 7.57-7.49 (m, 2H), 7.48- |
| | | 7.38 (m, 1H), 6.17-6.04 (m, 1H), 4.64-4.59 (m, 2H), 4.32-4.25 (m, 2H), |
| | | 3.66-3.58 (m, 2H), 3.25 (s, 3H), 3.12-3.07 (m, 2H), 3.06-2.99 (m, 3H), |
| | | 2.34 (d, J = 3.2 Hz, 1H), 2.31-2.22 (m, 1H) |
| I-1053 | 512.3 | 8.17-8.07 (m, 2H), 7.72-7.65 (m, 1H), 7.32-7.24 (m, 2H), 7.23-7.17 (m, |
| | | 2H), 6.42-6.35 (m, 1H), 6.30-6.24 (m, 1H), 4.63 (q, J = 6.8 Hz, 2H), 4.48- |
| | | 4.37 (m, 2H), 3.61 (td, J = 5.6, 18.2 Hz, 2H), 3.23 (s, 3H), 3.12 (td, J = 6.8, |
| | | 12.8 Hz, 2H), 2.94 (s, 3H), 2.93-2.86 (m, 3H), 2.37-2.25 (m, 2H), 2.11 (s, |
| | | 3H), 1.59-1.52 (m, 9H), 1.44 (s, 9H) |
| I-1054 | 499.2 | 2.24-2.34 (m, 2 H) 3.11 (d, J = 7.00 Hz, 2 H) 3.56-3.63 (m, 2 H) 3.70-3.72 |
| | | (m, 3 H) 4.04 (s, 6 H) 4.36-4.41 (m, 2 H) 4.62-4.66 (m, 2 H) 6.27 (s, 1 H) |
| | | 6.44-6.47 (m, 1 H) 7.05 (t, J = 7.36 Hz, 1 H) 7.09-7.16 (m, 2 H) 7.34-7.36 |
| | | (m, 1 H) 7.38-7.41 (m, 2 H) 7.70-7.73 (m, 1 H) 8.13-8.15 (m, 1 H) 11.54- |
| | | 11.60 (m, 1 H) |
| I-1055 | 517.3 | 11.62-11.54 (m, 1H), 8.15-8.10 (m, 1H), 7.73-7.67 (m, 1H), 7.43-7.34 |
| | | (m, 2H), 7.12-7.02 (m, 2H), 6.88 (dt, J = 2.4, 8.4 Hz, 1H), 6.49-6.42 (m, |
| | | 1H), 6.28 (br s, 1H), 4.68-4.60 (m, 2H), 4.44-4.35 (m, 2H), 3.77-3.71 (m, |
| | | 3H), 3.66-3.55 (m, 2H), 3.28-2.98 (m, 8H), 2.34 (br s, 2H) |
| I-1056 | 529.4 | 8.13-8.10 (m, 1H), 7.69 (d, J = 4.8 Hz, 1H), 7.60 (d, J = 1.6 Hz, 1H), 7.44- |
| | | 7.39 (m, 1H), 7.38-7.36 (m, 1H), 7.17-7.13 (m, 1H), 6.31-6.26 (m, 2H), |
| | | 6.25-6.22 (m, 1H), 6.03-5.82 (m, 1H), 4.66-4.61 (m, 2H), 4.39 (br d, J = |
| | | 11.2 Hz, 2H), 3.67 (d, J = 5.2 Hz, 3H), 3.62 (br t, J = 5.6 Hz, 1H), 3.57 (br t, |
| | | J = 5.6 Hz, 1H), 3.22 (br s, 3H), 3.17-3.14 (m, 1H), 3.10 (s, 1H), 2.99 (br s, |
| | | 3H), 2.75 (s, 3H), 2.34-2.22 (m, 2H) |
| I-1057 | 513.3 | 11.60-11.48 (m, 1H), 8.16-8.05 (m, 1H), 7.77-7.60 (m, 1H), 7.42-7.35 |
| | | (m, 1H), 7.21-7.15 (m, 2H), 7.12-7.03 (m, 2H), 6.47-6.41 (m, 1H), 6.31- |
| | | 6.24 (m, 1H), 4.69-4.59 (m, 2H), 4.44-4.35 (m, 2H), 3.72-3.66 (m, 3H), |
| | | 3.66-3.56 (m, 2H), 3.26-3.00 (m, 9H), 2.32-2.29 (m, 3H), 2.26 (d, J = 2.8 |
| | | Hz, 1H) |
| I-1058 | 533.2 | 11.65-11.58 (m, 1H), 8.16-8.09 (m, 1H), 7.67 (s, 1H), 7.47-7.40 (m, 2H), |
| | | 7.38-7.35 (m, 1H), 7.19 (d, J = 8.8 Hz, 1H), 7.15-7.11 (m, 1H), 6.47-6.44 |
| | | (m, 1H), 6.31 (s, 1H), 4.67-4.62 (m, 2H), 4.43-4.37 (m, 2H), 3.74-3.72 (m, |
| | | 3H), 3.65-3.57 (m, 2H), 3.24-3.04 (m, 9H), 2.30-2.23 (m, 1H) |
| I-1059 | 546.4 | 11.92-11.85 (m, 1H), 8.14-8.08 (m, 1H), 7.72-7.65 (m, 1H), 7.06 (d, J = |
| | | 8.4 Hz, 1H), 6.88-6.78 (m, 1H), 6.49 (s, 1H), 6.30 (s, 1H), 6.22 (dd, J = 1.6, |
| | | 8.4 Hz, 1H), 6.14-6.05 (m, 1H), 5.83-5.77 (m, 1H), 4.67-4.57 (m, 2H), |
| | | 4.39-4.29 (m, 2H), 3.68-3.66 (m, 3H), 3.60 (s, 2H), 3.23-3.01 (m, 8H), |
| | | 2.74 (d, J = 4.8 Hz, 3H), 2.36-2.33 (m, 1H), 2.27 (d, J = 2.0 Hz, 1H) |
| I-1060 | 500.3 | 11.82-11.60 (m, 1H), 8.52 (s, 1H), 8.32 (d, J = 4.8 Hz, 1H), 8.09 (s, 1H), |
| | | 7.73-7.65 (m, 1H), 7.50-7.44 (m, 1H), 7.42 (br d, J = 4.8 Hz, 1H), 7.23- |
| | | 7.17 (m, 1H), 6.56-6.52 (m, 1H), 6.34-6.28 (m, 1H), 4.72-4.58 (m, 2H), |
| | | 4.47-4.29 (m, 2H), 3.91-3.82 (m, 3H), 3.69-3.55 (m, 2H), 3.21-3.01 (m, |
| | | 6H), 2.68 (br s, 1H), 2.39-2.32 (m, 2H), 2.30-2.22 (m, 1H) |
| I-1061 | 517.3 | 11.73-11.66 (m, 1H), 8.20-8.09 (m, 1H), 7.73-7.67 (m, 1H), 7.50-7.43 |
| | | (m, 2H), 7.33-7.29 (m, 1H), 7.26 (br d, J = 8.0 Hz, 1H), 7.17-7.09 (m, 1H), |
| | | 6.45-6.41 (m, 1H), 6.35-6.30 (m, 1H), 4.68-4.62 (m, 2H), 4.45-4.37 (m, |
| | | 2H), 3.65-3.57 (m, 2H), 3.22-3.02 (m, 8H), 2.40-2.29 (m, 5H) |
| I-1062 | 533.3 | 11.73 (s, 1H), 8.15-8.10 (m, 1H), 7.72-7.67 (m, 1H), 7.53-7.44 (m, 2H), |
| | | 7.13 (s, 1H), 7.03 (br d, J = 6.4 Hz, 2H), 6.47 (s, 1H), 6.32 (br s, 1H), 4.67- |
| | | 4.61 (m, 2H), 4.45-4.37 (m, 2H), 3.80 (s, 3H), 3.59 (s, 2H), 3.11 (br s, 8H), |
| | | 2.34 (br d, J = 1.6 Hz, 2H) |
| I-1063 | 608.3 | 8.31-8.18 (m, 1H), 7.77-7.54 (m, 1H), 7.50-7.30 (m, 2H), 7.21-7.11 (m, |
| | | 1H), 6.86 (t, J = 7.6 Hz, 1H), 6.56-6.44 (m, 1H), 6.42-6.18 (m, 4H), 5.91 (d, |
| | | J = 4.4 Hz, 1H), 5.55-5.34 (m, 1H), 5.03-4.87 (m, 2H), 4.80-4.48 (m, 2H), |
| | | 4.44-4.24 (m, 2H), 3.68 (d, J = 5.2 Hz, 3H), 3.62 (d, J = 4.4 Hz, 1H), 3.30 (s, |
| | | 1H), 3.23 (s, 3H), 3.07-2.92 (m, 3H), 2.83 (s, 1H), 2.75 (s, 4H), 2.41-2.35 |
| | | (m, 1H), 2.29-2.19 (m, 1H) |
| I-1064 | 538.3 | 11.52-11.45 (m, 1H), 10.97 (s, 1H), 8.14-8.10 (m, 1H), 7.72-7.66 (m, 1H), |
| | | 7.45 (s, 1H), 7.39-7.34 (m, 1H), 7.24 (t, J = 2.4 Hz, 1H), 7.13-7.09 (m, 1H), |
| | | 7.06 (s, 1H), 6.47-6.44 (m, 1H), 6.38 (s, 1H), 6.28 (d, J = 4.0 Hz, 1H), 4.63 |
| | | (q, J = 6.8 Hz, 2H), 4.43-4.37 (m, 2H), 3.72-3.70 (m, 3H), 3.65-3.56 (m, |
| | | 2H), 3.20-2.99 (m, 8H), 2.37-2.28 (m, 2H) |
| I-1065 | 518.3 | 12.16-12.10 (m, 1H), 8.55-8.46 (m, 1H), 8.45-8.38 (m, 1H), 8.17-8.06 (m, |
| | | 1H), 7.74-7.65 (m, 1H), 7.26-7.17 (m, 1H), 7.04-6.91 (m, 1H), 6.54-6.45 |
| | | (s, 1H), 6.20-6.11 (m, 1H), 4.68-4.57 (m, 2H), 4.40-4.27 (m, 2H), 3.81 (s, |
| | | 3H), 3.70-3.54 (m, 1H), 3.24-3.96 (m, 9H), 2.39-2.24 (m, 1H) |
| I-1066 | 542.3 | 11.66-11.58 (m, 1H), 11.28 (s, 1H), 8.14-8.09 (m, 1H), 7.72-7.66 (m, 1H), |
| | | 7.63-7.58 (m, 2H), 7.46-7.41 (m, 2H), 7.15-7.09 (m, 1H), 6.49 (s, 1H), |
| | | 6.45-6.40 (m, 1H), 6.34-6.28 (m, 1H), 4.63 (q, J = 6.4 Hz, 2H), 4.44-4.38 |
| | | (m, 2H), 3.65-3.56 (m, 2H), 3.13 (td, J = 6.8, 19.2 Hz, 8H), 2.35-2.25 (m, |
| | | 2H) |
| I-1067 | 501.3 | 8.55 (s, 1H), 8.35 (d, J = 4.8 Hz, 1H), 8.15 (s, 1H), 8.11 (d, J = 4.0 Hz, 1H), |
| | | 7.87-7.78 (m, 1H), 7.69 (d, J = 4.8 Hz, 1H), 7.61-7.53 (m, 1H), 7.52-7.48 |
| | | (m, 1H), 7.45 (d, J = 10.4 Hz, 1H), 6.35 (br d, J = 13.2 Hz, 1H), 4.64 (br t, J = |
| | | 6.8 Hz, 2H), 4.41 (br d, J = 12.0 Hz, 2H), 3.87 (d, J = 3.2 Hz, 3H), 3.60 (td, J = |
| | | 5.6, 18.0 Hz, 2H), 3.25-3.08 (m, 6H), 2.99 (br s, 2H), 2.36-2.24 (m, 2H) |
| I-1068 | 565.2 | 12.25 (br s, 1H), 8.16-8.06 (m, 2H), 7.80-7.66 (m, 3H), 7.50 (br d, J = 7.6 |
| | | Hz, 1H), 7.09-6.98 (m, 1H), 6.36-6.25 (m, 1H), 6.21-6.11 (m, 1H), 4.64- |
| | | 4.58 (m, 2H), 4.44-4.27 (m, 2H), 3.65-3.55 (m, 2H), 3.30 (br s, 2H), 3.08 |
| | | (br dd, J = 6.0, 12.1 Hz, 4H), 3.04-2.99 (m, 2H), 2.74 (s, 3H), 2.33 (br s, 2H) |
| I-1069 | 436.5 | 12.03 (br s, 1H), 7.43-7.36 (m, 1H), 7.34 (br d, J = 7.6 Hz, 1H), 7.15 (d, J = |
| | | 8.4 Hz, 1H), 7.07-7.02 (m, 1H), 6.96-6.86 (m, 1H), 6.45 (br s, 1H), 6.16- |
| | | 6.08 (m, 1H), 4.35-4.30 (m, 2H), 3.72 (s, 3H), 3.61 (td, J = 5.6, 11.6 Hz, |
| | | 2H), 3.22-2.98 (m, 6H), 2.37 (br d, J = 2.4 Hz, 1H), 2.26 (br s, 1H), 2.07 (d, |
| | | J = 7.2 Hz, 3H). |
| I-1070 | 488.2 | |
| I-1071 | 509.1 | |
| I-1072 | 524.2 | |
| I-1073 | 554.1 | |
| I-1074 | 504.1 | |
| I-1075 | 524.2 | |
| I-1076 | 518.1 | |
| I-1077 | 474.1 | |
| I-1078 | 528.2 | |
| I-1079 | 509.1 | |
| I-1080 | 510.1 | |
| I-1081 | 556.1 | |
| I-1082 | 501.1 | |
| I-1083 | 502.1 | |
| I-1084 | 485.1 | |
| I-1085 | 524.2 | |
| I-1086 | 484.1 | |
| I-1087 | 569 | |
| I-1088 | 560.2 | |
| I-1089 | 522.1 | |
| I-1090 | 515.3 | 8.36 (s, 1H), 8.26 (s, 1H), 8.15 (d, J = 3.2 Hz), 7.72-7.50 (m, 1H), 7.68 (d, |
| | | J = 4.0 Hz), 7.48-7.35 (m, 2H), 6.40-6.32 (m, 1H), 4.63 (t, J = 2.4 Hz, 1H), |
| | | 4.44-4.32 (m, 1H), 3.75 (s, 3H), 3.64-3.55 (m, 2H), 3.19-3.07 (m, 6H), |
| | | 3.02-2.94 (m, 3H), 2.33-2.23 (m, 1H), 2.02 (s, 3H) |
| I-1091 | 418.4 | 11.61-11.46 (m, 1H), 7.47-7.31 (m, 3H), 7.19-7.01 (m, 3H), 6.53-6.43 |
| | | (m, 1H), 6.28 (br d, J = 1.2 Hz, 1H), 4.38 (br s, 2H), 3.72 (s, 3H), 3.63-3.53 |
| | | (m, 2H), 3.26-2.94 (m, 6H), 2.37-2.22 (m, 2H), 2.15-2.04 (m, 3H) |
| I-1092 | 465.4 | 11.92-11.82 (m, 1H), 7.09-7.03 (m, 1H), 6.87-6.78 (m, 1H), 6.56-6.44 |
| | | (m, 1H), 6.32-6.26 (m, 1H), 6.24-6.18 (m, 1H), 6.13-6.06 (m, 1H), 5.86- |
| | | 5.76 (m, 1H), 4.36-4.26 (m, 2H), 3.70-3.65 (m, 3H), 3.63-3.56 (m, 2H), |
| | | 3.30-3.07 (m, 6H), 2.74 (d, J = 4.8 Hz, 3H), 2.33 (br s, 2H), 2.07 (d, J = 7.6 |
| | | Hz, 3H) |
| I-1093 | 449.3 | 12.01 (br s, 1H), 7.33-7.23 (m, 2H), 7.09 (d, J = 8.0 Hz, 1H), 7.06-6.97 (m, |
| | | 2H), 6.50 (br s, 1H), 6.18-6.11 (m, 1H), 4.37-4.32 (m, 2H), 3.65-3.59 (m, |
| | | 2H), 3.23-2.99 (m, 6H), 2.45-2.42 (m, 6H), 2.38 (br s, 1H), 2.27 (br d, J = |
| | | 1.6 Hz, 1H), 2.08 (d, J = 4.4 Hz, 3H) |
| I-1094 | 437.2 | 12.11 (s, 1H), 8.50 (d, J = 5.6 Hz, 1H), 8.43-8.39 (m, 1H), 7.20 (d, J = 6.0 |
| | | Hz, 1H), 7.02-6.92 (m, 1H), 6.49 (s, 1H), 6.19-6.12 (m, 1H), 4.36-4.31 (m, |
| | | 2H), 3.82 (s, 3H), 3.61 (td, J = 5.6, 11.4 Hz, 2H), 3.21-3.00 (m, 6H), 2.37 (s, |
| | | 1H), 2.26 (d, J = 2.4 Hz, 1H), 2.07 (d, J = 6.4 Hz, 3H) |
| I-1095 | 464.3 | 12.04 (br s, 1H), 6.98-6.89 (m, 4H), 6.57 (br s, 1H), 6.17-6.09 (m, 1H), 4.35- |
| | | 4.30 (m, 2H), 4.28 (dd, J = 2.4, 5.2 Hz, 2H), 4.22-4.17 (m, 2H), 3.65-3.57 |
| | | (m, 2H), 3.25-2.97 (m, 6H), 2.39-2.25 (m, 2H), 2.07 (d, J = 6.8 Hz, 3H). |
| I-1096 | 447.1 | 8.12 (br d, J = 3.1 Hz, 1H), 7.70 (br d, J = 4.3 Hz, 1H), 7.63-7.43 (m, 2H), |
| | | 7.19 (br d, J = 8.5 Hz, 1H), 6.85-6.34 (m, 1H), 6.21-5.83 (m, 1H), 4.63 (br |
| | | d, J = 5.3 Hz, 2H), 4.30-3.97 (m, 4H), 3.74-3.51 (m, 2H), 3.17-3.06 (m, |
| | | 2H), 2.98 (s, 6H), 2.38-2.26 (m, 2H), 1.23-0.97 (m, 1H), 0.52-0.10 (m, |
| | | 4H) |
| I-1097 | 448.1 | 12.25 (br s, 1H), 7.67-7.48 (m, 4H), 6.92-6.75 (m, 1H), 6.44 (br d, J = 3.2 |
| | | Hz, 1H), 6.19-5.98 (m, 1H), 4.38-4.12 (m, 2H), 3.70-3.44 (m, 2H), 3.23- |
| | | 2.94 (m, 6H), 2.41-2.22 (m, 2H), 2.07 (d, J = 5.6 Hz, 3H), 2.02-1.94 (m, |
| | | 3H) |
| I-1098 | 457.3 | 12.47-12.09 (m, 1H), 9.39 (s, 1H), 8.67-8.46 (m, 1H), 8.34-8.07 (m, 1H), |
| | | 7.74-7.71 (m, 2H), 7.71-7.66 (m, 1H), 7.20-6.96 (m, 1H), 6.29 (d, J = 3.2 |
| | | Hz, 1H), 6.24-6.17 (m, 1H), 4.38 (br d, J = 8.5 Hz, 2H), 3.64-3.58 (m, 2H), |
| | | 3.01 (td, J = 2.4, 3.6 Hz, 6H), 2.37 (br d, J = 3.2 Hz, 1H), 2.29-2.23 (m, 1H), |
| | | 2.07 (d, J = 6.0 Hz, 3H) |
| I-1099 | 449.2 | 12.08 (br s, 1H), 7.55-7.53 (m, 1H), 7.52 (br s, 2H), 7.47-7.42 (m, 2H), |
| | | 7.23-7.17 (m, 1H), 7.02-6.93 (m, 1H), 6.63-6.59 (m, 1H), 6.19-6.13 (m, |
| | | 1H), 4.36-4.31 (m, 2H), 3.64-3.58 (m, 2H), 3.17 (br s, 3H), 3.10-2.99 (m, |
| | | 3H), 2.39-2.26 (m, 2H), 2.08 (d, J = 2.8 Hz, 3H) |
| I-1100 | 542.2 | 11.46 (s, 1H), 8.67 (dd, J = 2.0, 6.0 Hz, 1H), 8.37 (s, 1H), 8.11 (d, J = 11.6 |
| | | Hz, 1H), 7.73-7.68 (m, 1H), 7.68-7.61 (m, 1H), 7.59 (s, 1H), 7.31 (s, 1H), |
| | | 6.21 (s, 1H), 4.63 (q, J = 6.8 Hz, 2H), 4.37 (d, J = 18.8 Hz, 2H), 3.73-3.58 |
| | | (m, 2H), 3.31 (s, 3H), 3.16-3.09 (m, 2H), 3.08-2.97 (m, 3H), 2.37 (d, J = |
| | | 2.8 Hz, 1H), 2.31 (s, 3H), 2.23 (s, 1H) |
| I-1101 | 541.4 | 12.20 (d, J = 4.4 Hz, 1H), 11.38 (s, 1H), 8.50-8.44 (m, 1H), 8.15-8.09 (m, |
| | | 2H), 7.72-7.66 (m, 1H), 7.30 (s, 1H), 7.14-7.04 (m, 1H), 6.88 (s, 1H), 6.20 |
| | | (s, 1H), 4.68-4.60 (m, 2H), 4.44-4.34 (m, 2H), 3.70-3.59 (m, 2H), 3.19 (d, |
| | | J = 2.0 Hz, 2H), 3.14-3.08 (m, 3H), 3.08-3.00 (m, 2H), 2.47-2.21 (m, 6H) |
| I-1103 | 500.2 | 8.11 (d, J = 3.2 Hz, 1H), 7.78-7.67 (m, 2H), 7.50-7.40 (m, 4H), 7.18 (d, J = |
| | | 8.4 Hz, 1H), 7.08 (dt, J = 0.8, 7.6 Hz, 1H), 6.39-6.27 (m, 1H), 4.63 (dt, J = |
| | | 2.8, 6.8 Hz, 2H), 4.41 (dd, J = 1.2, 12.0 Hz, 2H), 3.72 (d, J = 3.6 Hz, 3H), |
| | | 3.60 (td, J = 5.6, 18.4 Hz, 2H), 3.21-3.08 (m, 5H), 2.98 (s, 3H), 2.36-2.23 |
| | | (m, 2H) |
| I-1104 | 437.2 | 7.55-7.49 (m, 1H), 7.49-7.43 (m, 1H), 7.43-7.40 (m, 1H), 7.40-7.29 (m, |
| | | 1H), 7.18 (d, J = 8.4 Hz, 1H), 7.08 (dt, J = 1.6, 7.3 Hz, 1H), 6.18-6.12 (m, |
| | | 1H), 4.32 (br s, 2H), 3.73 (s, 3H), 3.64-3.57 (m, 2H), 3.20 (br s, 3H), 2.99 (br |
| | | s, 3H), 2.37 (br d, J = 3.2 Hz, 1H), 2.38-2.24 (m, 1H), 2.07 (d, J = 7.6 Hz, |
| | | 3H) |
| I-1105 | 454.9 | 7.56-7.52 (m, 1H), 7.52-7.46 (m, 1H), 7.44-7.30 (m, 1H), 7.05 (d, J = 8.4 |
| | | Hz, 1H), 6.98 (t, J = 8.8 Hz, 1H), 6.20-6.05 (m, 1H), 4.31 (s, 2H), 3.74 (s, |
| | | 3H), 3.65-3.53 (m, 2H), 3.23-2.90 (m, 6H), 2.41-2.21 (m, 2H), 2.07 (d, J = |
| | | 7.2 Hz, 3H) |
| I-1106 | 601.2 | 7.79 (dd, J = 1.2, 4.8 Hz, 1H), 7.49-7.44 (m, 1H), 7.38-7.25 (m, 4H), 7.19 |
| | | (d, J = 8.4 Hz, 1H), 7.12-7.06 (m, 1H), 6.92 (dd, J = 4.8, 8.0 Hz, 1H), 6.26 |
| | | (d, J = 1.6 Hz, 1H), 4.36 (s, 2H), 3.80 (s, 3H), 3.75 (d, J = 4.8 Hz, 4H), 3.74 |
| | | (s, 3H), 3.65-3.58 (m, 2H), 3.38-3.33 (m, 4H), 2.41-2.25 (m, 2H), 2.08 (d, |
| | | J = 7.2 Hz, 3H) |
| I-1107 | 701.3 | 12.17 (d, J = 7.2 Hz, 1H), 8.18-8.08 (m, 1H), 7.72-7.65 (m, 1H), 7.47 (d, J = |
| | | 8.8 Hz, 1H), 7.37 (s, 1H), 7.05 (s, 1H), 6.97-6.85 (m, 2H), 6.46 (s, 1H), |
| | | 6.29 (d, J = 8.4 Hz, 1H), 6.17-6.10 (m, 1H), 4.63 (d, J = 7.6 Hz, 2H), 4.38- |
| | | 4.29 (m, 2H), 3.87 (s, 3H), 3.82-3.77 (m, 4H), 3.77-3.75 (m, 3H), 3.66- |
| | | 3.59 (m, 2H), 3.51 (s, 4H), 3.10 (d, J = 7.6 Hz, 2H), 2.34 (dd, J = 2.4, 6.4 Hz, |
| | | 2H) |
| I-1108 | 681.2 | 12.21 (d, J = 8.8 Hz, 1H), 8.47 (d, J = 5.6 Hz, 1H), 8.44 (s, 1H), 8.28 (s, 1H), |
| | | 8.22-8.12 (m, 1H), 8.11-8.08 (m, 1H), 7.72-7.67 (m, 1H), 7.20 (d, J = 5.6 |
| | | Hz, 1H), 7.09-6.98 (m, 1H), 6.56 (d, J = 2.0 Hz, 1H), 6.22-6.11 (m, 1H), |
| | | 4.66-4.59 (m, 2H), 4.41-4.31 (m, 2H), 4.21-4.13 (m, 2H), 3.85 (s, 3H), |
| | | 3.76 (d, J = 12.8 Hz, 8H), 3.66 (t, J = 5.2 Hz, 1H), 3.61 (t, J = 5.6 Hz, 1H), |
| | | 3.14-3.07 (m, 2H), 2.37-2.28 (m, 2H), 1.25-1.17 (m, 3H) |
| I-1109 | 708.4 | 11.99 (d, J = 6.4 Hz, 1H), 8.14-8.07 (m, 1H), 7.75-7.60 (m, 1H), 7.14 (d, J = |
| | | 8.4 Hz, 1H), 6.89-6.79 (m, 1H), 6.43-6.36 (m, 3H), 6.13-6.06 (m, 1H), |
| | | 4.65-4.59 (m, 2H), 4.37-4.28 (m, 2H), 3.71 (d, J = 3.6 Hz, 3H), 3.65-3.57 |
| | | (m, 6H), 3.09 (q, J = 7.2 Hz, 2H), 2.97 (s, 6H), 2.55 (s, 4H), 2.35-2.25 (m, |
| | | 2H), 1.64 (dt, J = 3.2, 6.4 Hz, 1H), 0.45-0.32 (m, 4H) |
| I-1110^{j} | 643.2 | |
| I-1111 | 753.3 | |
| I-1112 | 741.3 | |
| I-1113 | 719.3 | |
| I-1114 | 747.3 | |
| I-1115 | 733.4 | |
| I-1116 | 657.3 | |
| I-1117 | 735.3 | |
| I-1118 | 730.4 | |
| I-1119 | 704.2 | |
| I-1120 | 730.2 | |
| I-1121 | 766.3 | |
| I-1122 | 752.2 | |
| I-1123 | 752.2 | |
| I-1124 | 736.3 | |
| I-1125 | 729.3 | |
| I-1126 | 659.2 | |
| I-1127 | 725.4 | |
| I-1128 | 729.3 | |
| I-1129 | 709.3 | |
| I-1130 | 709.3 | |
| I-1131 | 698.4 | |
| I-1132 | 748.2 | |
| I-1133 | 700.3 | |
| I-1134 | 643.3 | |
| I-1135 | 657.3 | |
| I-1136 | 760.2 | |
| I-1137 | 753.3 | |
| I-1138 | 767.3 | 12.35-12.18 (m, 1H), 8.46-8.43 (m, 1H), 8.16-8.07 (m, 1H), 7.92-7.85 |
| | | (m, 1H), 7.80 (d, J = 2.4 Hz, 1H), 7.76-7.64 (m, 1H), 7.38 (dd, J = 2.4, 10.4 |
| | | Hz, 1H), 7.07-6.94 (m, 2H), 6.82 (d, J = 2.8 Hz, 1H), 6.18 (d, J = 8.8 Hz, |
| | | 1H), 4.63 (q, J = 6.8 Hz, 2H), 4.41-4.32 (m, 2H), 4.22 (d, J = 11.6 Hz, 2H), |
| | | 3.89-3.79 (m, 7H), 3.68-3.59 (m, 4H), 3.27 (s, 4H), 3.10 (q, J = 6.8 Hz, |
| | | 2H), 2.45 (dd, J = 11.2, 12.4 Hz, 2H), 2.39-2.26 (m, 2H), 1.19 (d, J = 6.0 Hz, |
| | | 6H) |
| I-1139 | 692.3 | |
| I-1140 | 718.3 | |
| I-1141 | 682.3 | 12.28-12.04 (m, 1H), 8.19-8.07 (m, 1H), 7.86-7.78 (m, 1H), 7.73-7.66 |
| | | (m, 1H), 7.39 (dd, J = 2.4, 10.4 Hz, 1H), 6.97-6.78 (m, 2H), 6.37-6.00 (m, |
| | | 2H), 4.63 (q, J = 6.8 Hz, 2H), 4.37-4.25 (m, 2H), 3.86 (s, 3H), 3.81 (s, 4H), |
| | | 3.59 (t, J = 5.6 Hz, 2H), 3.28 (s, 4H), 3.18 (d, J = 6.0 Hz, 1H), 3.13-3.06 (m, |
| | | 2H), 2.56 (s, 2H), 2.34 (s, 1H), 2.26 (d, J = 3.2 Hz, 1H), 2.16-1.93 (m, 3H), |
| | | 1.88-1.65 (m, 1H) |
| I-1142 | 720.3 | |
| I-1143 | 768.3 | |
| I-1144 | 768.3 | |
| I-1145 | 751.3 | |
| I-1146 | 723.6 | |
| I-1147 | 713.3 | |
| I-1148 | 725.3 | |
| I-1149 | 641.2 | 12.41-12.27 (m, 1H), 8.53 (d, J = 5.2 Hz, 1H), 8.40 (br s, 1H), 7.78 (d, J = |
| | | 2.4 Hz, 1H), 7.73-7.67 (m, 1H), 7.44-7.37 (m, 2H), 7.37-7.34 (m, 1H), |
| | | 6.97-6.87 (m, 1H), 6.34 (br s, 1H), 6.21-6.13 (m, 2H), 4.36 (br t, J = 6.8 Hz, |
| | | 3H), 4.33 (br s, 1H), 3.83 (s, 3H), 3.76 (br s, 4H), 3.64 (br t, J = 5.6 Hz, 1H), |
| | | 3.58-3.56 (m, 1H), 3.23 (br s, 5H), 2.97 (q, J = 6.8 Hz, 2H), 2.49-2.45 (m, |
| | | 1H), 2.33 (br s, 1H), 2.26 (br s, 1H), 1.01 (t, J = 7.6 Hz, 3H) |
| I-1150 | 668.2 | 12.15-12.08 (m, 1H), 8.18 (s, 1H), 8.13 (s, 1H), 7.85-7.78 (m, 1H), 7.74- |
| | | 7.63 (m, 1H), 7.28 (d, J = 7.2 Hz, 1H), 6.93 (dd, J = 4.8, 8.0 Hz, 1H), 6.84 (d, |
| | | J = 6.0 Hz, 1H), 6.87-6.75 (m, 1H), 6.09 (br s, 1H), 5.86 (br s, 1H), 4.63 (q, J = |
| | | 6.4 Hz, 2H), 4.37-4.26 (m, 2H), 3.87 (br s, 2H), 3.82 (s, 3H), 3.82-3.74 |
| | | (m, 2H), 3.69-3.55 (m, 4H), 3.15-3.06 (m, 4H), 3.02 (br d, J = 4.8 Hz, 2H), |
| | | 2.93 (br d, J = 16.6 Hz, 1H), 2.69 (br dd, J = 3.6, 10.8 Hz, 1H), 2.40-2.34 (m, |
| | | 2H), 2.31 (s, 3H), 2.27 (br s, 1H), 0.86 (d, J = 6.8 Hz, 3H) |
| I-1151 | 683.2 | |
| I-1152 | 730.5 | |
| I-1153 | 742.3 | |
| I-1154 | 752.5 | |
| I-1155 | 659.2 | |
| I-1156 | 659.2 | |
| I-1157 | 693.2 | |
| I-1158 | 693.3 | |
| I-1159 | 683.2 | |
| I-1160 | 703.2 | |
| I-1161 | 691.3 | 12.37-12.21 (m, 1H), 8.17-8.07 (m, 1H), 7.85-7.76 (m, 1H), 7.74-7.65 |
| | | (m, 1H), 7.55 (s, 1H), 7.49 (dd, J = 7.6, 11.2 Hz, 2H), 7.39 (dd, J = 2.4, |
| | | 10.4Hz, 1H), 7.32-7.22 (m, 3H), 7.21-7.16 (m, 1H), 6.24-6.12 (m, 1H), |
| | | 4.69-4.58 (m, 2H), 4.44-4.30 (m, 2H), 4.03 (s, 2H), 3.86 (s, 7H), 3.71-3.60 |
| | | (m, 2H), 3.31-3.28 (m, 3H), 3.25-3.17 (m, 1H), 3.12 (q, J = 7.2 Hz, 2H), |
| | | 2.40-2.28 (m, 2H) |
| I-1162 | 651.5 | |
| I-1163 | 685.3 | |
| I-1164 | 724.3 | |
| I-1165 | 724.3 | |
| I-1166 | 753.4 | |
| I-1167 | 751.2 | |
| I-1168 | 667.4 | 12.10-12.04 (m, 1H), 8.15-8.08 (m, 1H), 7.81 (dd, J = 1.2, 4.8 Hz, 1H), |
| | | 7.72-7.65 (m, 1H), 7.28 (dd, J = 1.2, 8.0 Hz, 1H), 6.93 (dd, J = 4.8, 8.0 Hz, |
| | | 1H), 6.80-6.52 (m, 2H), 6.09 (br d, J = 4.0 Hz, 1H), 5.88-5.85 (m, 1H), 4.65- |
| | | 4.60 (m, 2H), 4.35-4.28 (m, 2H), 3.86-3.78 (m, 8H), 3.66-3.58 (m, 2H), |
| | | 3.36 (br s, 6H), 3.12-3.06 (m, 2H), 2.75-2.65 (m, 1H), 2.38-2.31 (m, 2H), |
| | | 2.28-2.12 (m, 4H), 1.86-1.60 (m, 6H), 1.28-1.13 (m, 1H), 0.85-0.80 (m, |
| | | 2H), 0.74-0.68 (m, 2H) |
| I-1169 | 637.3 | 12.35 (s, 1H), 8.53 (d, J = 5.2 Hz, 1H), 8.42 (s, 1H), 7.78 (d, J = 2.4 Hz, 1H), |
| | | 7.43 (d, J = 5.2 Hz, 1H), 7.36 (dd, J = 2.4, 10.4 Hz, 1H), 6.98-6.67 (m, 2H), |
| | | 6.39-6.32 (m, 1H), 6.26-6.15 (m, 1H), 4.44 (s, 2H), 3.83 (s, 3H), 3.79-3.74 |
| | | (m, 4H), 3.73-3.68 (m, 2H), 3.26-3.20 (m, 6H), 2.41-2.33 (m, 2H), 1.06- |
| | | 0.96 (m, 3H) |
| I-1170 | 659.6 | 12.68-11.85 (m, 1H), 8.53 (d, J = 5.2 Hz, 1H), 8.44-8.37 (m, 1H), 7.78 (d, J = |
| | | 1.6 Hz, 1H), 7.47-7.40 (m, 1H), 7.38-7.29 (m, 1H), 7.01-6.82 (m, 1H), |
| | | 6.38-6.30 (m, 1H), 6.26-6.10 (m, 1H), 4.37 (s, 2H), 3.83 (s, 3H), 3.76 (s, |
| | | 4H), 3.63 (t, J = 5.6 Hz, 2H), 3.23 (s, 6H), 2.65-2.59 (m, 2H), 2.44-2.31 (m, |
| | | 4H), 1.05-0.97 (m, 3H) |
| I-1171 | 665.3 | 11.53 (s, 1H), 8.18-8.04 (m, 1H), 7.70-7.63 (m, 2H), 7.40-7.35 (m, 1H), |
| | | 7.30-7.24 (m, 2H), 7.13 (d, J = 8.4 Hz, 1H), 7.03 (t, J = 7.2 Hz, 1H), 6.90- |
| | | 6.80 (m, 2H), 6.11-6.05 (m, 1H), 6.03-5.99 (m, 1H), 4.67 (s, 2H), 4.65- |
| | | 4.59 (m, 2H), 4.37-4.27 (m, 2H), 3.95 (s, 2H), 3.78 (s, 3H), 3.72-3.67 (m, |
| | | 3H), 3.67-3.54 (m, 5H), 3.08 (q, J = 7.2 Hz, 2H), 2.42-2.20 (m, 3H) |
| I-1172 | 685.4 | 12.20-11.91 (m, 1H), 8.21-7.98 (m, 1H), 7.81 (d, J = 2.4 Hz, 1H), 7.73- |
| | | 7.65 (m, 1H), 7.38 (dd, J = 2.4, 10.4 Hz, 1H), 6.73-6.60 (m, 1H), 6.52 (s, |
| | | 1H), 6.07 (s, 1H), 4.63 (q, J = 6.8 Hz, 2H), 4.39-4.26 (m, 2H), 3.86 (s, 3H), |
| | | 3.80 (s, 4H), 3.69-3.55 (m, 2H), 3.28 (s, 4H), 3.15-3.02 (m, 2H), 2.40-2.27 |
| | | (m, 2H), 2.24 (d, J = 15.6 Hz, 2H), 2.13 (s, 2H), 1.81-1.66 (m, 6H), 0.86- |
| | | 0.79 (m, 3H) |
| I-1173 | 723.6 | 7.78 (d, J = 2.4 Hz, 1H), 7.73-7.65 (m, 2H), 7.39-7.33 (m, 1H), 7.24-7.19 |
| | | (m, 1H), 6.94-6.88 (m, 1H), 6.78 (s, 1H), 6.06-5.96 (m, 1H), 4.39-4.28 (m, |
| | | 1H), 4.13-4.05 (m, 3H), 3.85-3.82 (m, 3H), 3.69-3.51 (m, 8H), 3.42 (d, J = |
| | | 5.6 Hz, 1H), 3.40-3.35 (m, 1H), 3.24 (s, 4H), 2.56-2.54 (m, 3H), 2.40 (s, |
| | | 2H), 2.36-2.24 (m, 2H), 1.18 (q, J = 7.2 Hz, 3H), 1.03 (t, J = 7.6 Hz, 3H), |
| | | 0.57-0.44 (m, 1H), 0.15-0.04 (m, 2H), −0.16-−0.27 (m, 2H) |
| I-1174 | 687.4 | 12.17-11.90 (m, 1H), 8.21-8.05 (m, 1H), 7.81 (d, J = 2.4 Hz, 1H), 7.74-7.63 |
| | | (m, 1H), 7.49-7.27 (m, 1H), 6.88 (s, 1H), 6.75-6.56 (m, 1H), 6.15-5.94 (m, |
| | | 1H), 4.72-4.57 (m, 2H), 4.42-4.25 (m, 2H), 3.87 (s, 3H), 3.84 (s, 3H), 3.71- |
| | | 3.54 (m, 3H), 3.16-3.03 (m, 3H), 2.34 (d, J = 2.4 Hz, 2H), 2.31-2.23 (m, 1H), |
| | | 2.02-1.92 (m, 1H), 1.89-1.78 (m, 3H), 1.75-1.58 (m, 2H), 1.57-1.36 (m, 4H), |
| | | 1.36-1.20 (m, 2H), 1.01-0.72 (m, 2H), 0.58 (t, J = 7.2 Hz, 3H) |
| I-1175 | 687.4 | 12.20-11.80 (m, 1H), 8.19-8.04 (m, 1H), 7.81 (d, J = 2.4 Hz, 1H), 7.75-7.65 |
| | | (m, 1H), 7.44-7.32 (m, 1H), 6.88 (s, 1H), 6.73-6.60 (m, 1H), 6.19-5.87 (m, |
| | | 1H), 4.70-4.59 (m, 2H), 4.38-4.23 (m, 2H), 3.87 (s, 3H), 3.83 (d, J = 5.2 Hz, |
| | | 3H), 3.71-3.51 (m, 3H), 3.15-3.04 (m, 3H), 2.34 (d, J = 1.6 Hz, 2H), 2.27 (d, |
| | | J = 2.4 Hz, 1H), 2.02-1.93 (m, 1H), 1.91-1.80 (m, 3H), 1.78-1.64 (m, 2H), 1.62- |
| | | 1.42 (m, 4H), 1.36-1.20 (m, 2H), 0.96-0.76 (m, 2H), 0.58 (t, J = 7.2 Hz, 3H) |
| I-1176 | 516.1 | 12.41 (s, 1H), 7.60 (t, J = 1.6 Hz, 1H), 7.36-7.24 (m, 1H), 7.11-6.97 (m, |
| | | 1H), 6.93-6.81 (m, 1H), 6.49 (s, 1H), 6.31-6.13 (m, 1H), 5.16-4.49 (m, |
| | | 4H), 4.38-4.30 (m, J = 4.4 Hz, 2H), 4.23-3.95 (m, 4H), 3.73-3.47 (m, 2H), |
| | | 2.39-2.25 (m, 2H), 2.08 (d, J = 4.8 Hz, 3H), 1.38-1.24 (m, 6H) |

^{j}LCMS reported as (M − 18)⁺.

TABLE 10
Compounds synthesized via Method 2, the cross coupling
of the corresponding chlorides and boronic acids

| | | | LCMS | |
|---|---|---|---|---|
| | | | (ESI+) | |
| | | Boronic | m/z | |
| I-# | Chloride | Acid | (M + H)+ | ¹H NMR (400 MHz, DMSO-d₆) δ |
| I-1220^{d, k} | UY | QD | 597.3 | 12.42-12.24 (m, 1H), 8.18-8.07 (m, 1H), 7.74- |
| | | | | 7.62 (m, 1H), 7.45-7.37 (m, 1H), 7.33-7.24 (m, |
| | | | | 1H), 7.19-6.94 (m, 3H), 6.33-5.98 (m, 1H), |
| | | | | 5.06 (br s, 1H), 4.84 (br d, J = 14.8 Hz, 2H), 4.73- |
| | | | | 4.52 (m, 3H), 4.43-4.28 (m, 2H), 4.17-3.98 |
| | | | | (m, 4H), 3.67-3.55 (m, 2H), 3.14-3.07 (m, 2H), |
| | | | | 2.40-2.25 (m, 2H), 1.45-1.12 (m, 6H) |
| I-1211 | UD | PW | 695.1 | 8.14-8.08 (m, 1H), 7.92-7.84 (m, 1H), 7.80- |
| | | | | 7.76 (m, 1H), 7.72-7.63 (m, 1H), 7.63-7.55 (m, |
| | | | | 2H), 7.39-7.33 (m, 1H), 7.26-7.20 (m, 2H), |
| | | | | 6.39-6.31 (m, 1H), 4.66-4.60 (m, 2H), 4.46- |
| | | | | 4.39 (m, 2H), 3.85-3.83 (m, 3H), 3.80-3.66 (m, |
| | | | | 2H), 3.63-3.51 (m, 4H), 3.27 (br s, 5H), 3.18- |
| | | | | 3.13 (m, 1H), 3.11-3.07 (m, 1H), 2.55-2.53 (m, |
| | | | | 4H), 2.23 (br s, 2H), 1.06-1.01 (m, 3H) |

^{k}Instead of XPhos, CataCXium ® A PD G3 was used as the catalyst.

TABLE 11
Compounds synthesized via Method 2, coupling of
the corresponding halogens and boronic acids

| | LCMS | |
|---|---|---|
| | (ESI+) | |
| | m/z | |
| I-# | (M + H)⁺ | ¹H NMR (400 MHz, DMSO-d₆) δ |
| | 717.3 | 12.13 (br d, J = 6.4 Hz, 1H), 8.05-7.93 (m, 1H), 7.71-7.62 (m, 1H), |
| | | 7.58-7.50 (m, 1H), 7.45-7.40 (m, 1H), 7.32-7.20 (m, 2H), 7.17-7.07 |
| | | (m, 2H), 6.97-6.83 (m, 1H), 6.80-6.73 (m, 1H), 6.69 (d, J = 1.6 Hz, |
| | | 1H), 6.07-5.95 (m, 1H), 4.56-4.44 (m, 2H), 4.27-4.16 (m, 2H), 4.01- |
| | | 3.91 (m, 2H), 3.87 (br t, J = 5.2 Hz, 2H), 3.74 (br s, 2H), 3.56-3.44 (m, |
| | | 2H), 2.96 (q, J = 7.2 Hz, 2H), 2.53 (br t, J = 7.2 Hz, 2H), 2.25-2.13 (m, |
| | | 2H), 1.64-1.55 (m, 4H), 1.39 (br s, 2H), 1.29 (br s, 4H), 1.19-−0.02 |
| | | (m, 2H) |
| I-1231 | 558.2 | 12.24 (d, J = 4.4 Hz, 1H), 8.53 (d, J = 5.2 Hz, 1H), 8.40 (s, 1H), 7.85- |
| | | 7.72 (m, 1H), 7.45-7.32 (m, 2H), 7.20-7.10 (m, 1H), 6.97-6.84 (m, |
| | | 1H), 6.61-6.53 (m, 1H), 6.35 (s, 1H), 6.24-6.11 (m, 1H), 4.43-4.33 |
| | | (m, 4H), 3.65 (t, J = 5.6 Hz, 1H), 3.60 (t, J = 5.6 Hz, 2H), 3.15-2.98 (m, |
| | | 8H), 2.38-2.31 (m, 2H), 2.31-2.23 (m, 1H), 1.00 (t, J = 7.6 Hz, 3H) |

^{l}The product of the coupling was further hydrolyzed with LiOH in THE at rt for 1 hr.

### Example I-356 (Method 3): Synthesis of 3-(4-(6-(1-(3-(1H-1,2,3-triazol-1-yl)propanoyl)-1,2,5,6-tetrahydropyridin-3-yl)-7-fluoro-4-(2-methoxyphenyl)-1H-indole-2-carbonyl)piperazin-1-yl)-4-methoxybenzonitrile

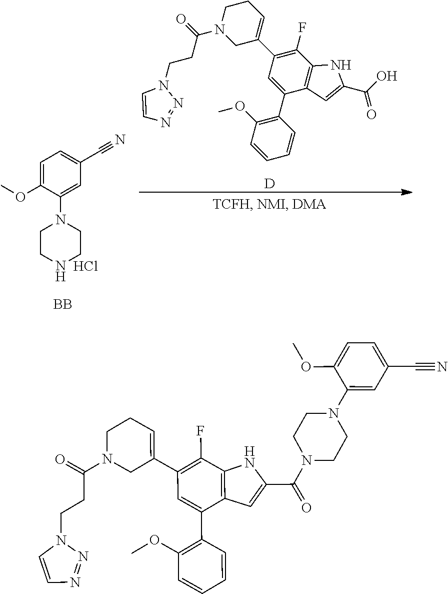

To a mixture of 7-fluoro-4-(2-methoxyphenyl)-6-[1-[3-(triazol-1-yl)propanoyl]-3,6-dihydro-2H-pyridin-5-yl]-1H-indole-2-carboxylic acid (50 mg, 1.0 eq, Intermediate D) in DMA (2 mL) was added NMI (25 mg, 3.0 eq) and TCFH (1.1 eq). Then 4-methoxy-3-(piperazin-1-yl)benzonitrile (26 mg, 1.2 eq, Intermediate BB) was added to the mixture at 25° C. and the mixture was stirred at 25° C. for 12 hrs. On completion, the reaction mixture was concentrated under reduced pressure to give a residue. The residue was purified by prep-HPLC (FA condition, neutral condition) to give the title compound. LC-MS (ESI⁺) m/z 689.3; ¹H NMR (400 MHz, DMSO-d6) δ=12.15 (br s, 1H), 8.15-8.08 (m, 1H), 7.73-7.65 (m, 1H), 7.49 (dd, J=1.6, 8.4 Hz, 1H), 7.44-7.32 (m, 2H), 7.28 (d, J=1.5 Hz, 1H), 7.19-7.10 (m, 2H), 7.06 (t, J=7.3 Hz, 1H), 7.00-6.88 (m, 1H), 6.44 (br s, 1H), 6.19-6.09 (m, 1H), 4.67-4.58 (m, 2H), 4.40-4.30 (m, 2H), 3.88 (s, 3H), 3.82 (br s, 4H), 3.77-3.71 (m, 3H), 3.66 (br t, J=5.8 Hz, 1H), 3.60 (br t, J=5.6 Hz, 1H), 3.16-2.97 (m, 6H), 2.40-2.22 (m, 2H).

TABLE 12
Compounds synthesized via Method 3, coupling of
the corresponding amines and carboxylic acids.

| | | | LCMS | |
|---|---|---|---|---|
| | | | (ESI+) | |
| | | | m/z | |
| I-# | Amine | Acid | (M + H)⁺ | ¹H NMR (400 MHz, DMSO-d₆) δ |
| I-380 | CJ | D | 612.3 | |
| I-381 | CK | D | 650.2 | |
| I-324 | O | D | 669.2 | |
| I-325 | P | D | 598.2 | |
| I-339 | V | D | 700.3 | 12.17 (br d, J = 6.5 Hz, 1H), 8.16-8.07 (m, 1H), 7.88 (d, J = 8.0 |
| | | | | Hz, 1H), 7.78 (br d, J = 7.9 Hz, 1H), 7.73-7.61 (m, 2H), 7.60- |
| | | | | 7.54 (m, 1H), 7.44-7.34 (m, 2H), 7.16 (br d, J = 8.3 Hz, 1H), |
| | | | | 7.06 (t, J = 7.3 Hz, 1H), 7.01-6.90 (m, 1H), 6.49 (br s, 1H), 6.15 |
| | | | | (br d, J = 10.9 Hz, 1H), 4.63 (q, J = 7.3 Hz, 2H), 4.42-4.29 (m, |
| | | | | 2H), 3.91 (br s, 4H), 3.75 (d, J = 3.0 Hz, 3H), 3.70-3.57 (m, |
| | | | | 2H), 3.41 (br s, 4H), 3.15-3.06 (m, 2H), 2.69 (s, 3H), 2.41-2.23 |
| | | | | (m, 2H) |
| I-340 | W | D | 748.3 | 12.16 (br d, J = 7.0 Hz, 1H), 8.17-8.05 (m, 1H), 7.75-7.64 (m, |
| | | | | 1H), 7.48-7.29 (m, 2H), 7.17 (br d, J = 8.3 Hz, 1H), 7.06 (t, J = |
| | | | | 7.3 Hz, 1H), 7.00-6.86 (m, 2H), 6.74 (br d, J = 13.4 Hz, 1H), |
| | | | | 6.46 (br s, 1H), 6.19-6.08 (m, 1H), 4.80-4.56 (m, 3H), 4.44- |
| | | | | 4.25 (m, 2H), 3.85 (br s, 4H), 3.75 (d, J = 3.0 Hz, 3H), 3.69-3.58 |
| | | | | (m, 2H), 3.21 (br s, 4H), 3.14-3.06 (m, 2H), 2.51-2.48 (m, 3H), |
| | | | | 2.39-2.24 (m, 2H), 1.53 (br d, J = 6.9 Hz, 6H) |
| I-341 | X | D | 702.3 | |
| I-342 | Y | D | 714.3 | |
| I-355 | BA | D | 686.3 | |
| I-357 | BC | D | 700.3 | |
| I-358 | BD | D | 701.3 | 12.14 (br s, 1H), 8.18-8.05 (m, 2H), 7.72-7.66 (m, 1H), 7.51 |
| | | | | (d, J = 7.8 Hz, 1H), 7.44-7.33 (m, 2H), 7.18-7.14 (m, 1H), |
| | | | | 7.08-6.89 (m, 3H), 6.46 (br s, 1H), 6.19-6.08 (m, 1H), 4.67- |
| | | | | 4.58 (m, 2H), 4.41-4.30 (m, 2H), 3.81 (br s, 4H), 3.73 (d, J = 3.3 |
| | | | | Hz, 3H), 3.68-3.58 (m, 2H), 3.38 (br s, 5H), 3.10 (br d, J = 6.8 |
| | | | | Hz, 2H), 2.41-2.23 (m, 2H) |
| I-359 | BE | D | 665.3 | |
| I-360 | BF | D | 723.3 | |
| I-383 | CN | D | 674.4 | |
| I-361 | BG | D | 653.3 | |
| I-362 | BH | D | 686.3 | |
| I-363 | BI | D | 685.3 | |
| I-384 | CO | D | 677.3 | |
| I-385 | CP | D | 663.3 | |
| I-386 | CQ | D | 720.3 | |
| I-401 | CM | D | 699.3 | 12.22-12.03 (m, 2H), 8.18-8.06 (m, 1H), 7.95 (s, 1H), 7.80 (d, J = |
| | | | | 3.3 Hz, 1H), 7.74-7.62 (m, 1H), 7.46-7.32 (m, 2H), 7.16 (br |
| | | | | d, J = 8.1 Hz, 1H), 7.06 (t, J = 7.4 Hz, 1H), 7.00-6.88 (m, 1H), |
| | | | | 6.60-6.43 (m, 2H), 6.21-6.10 (m, 1H), 4.70-4.54 (m, 2H), 4.45- |
| | | | | 4.28 (m, 2H), 3.90 (br s, 4H), 3.78-3.69 (m, 3H), 3.68-3.58 |
| | | | | (m, 2H), 3.10 (q, J = 6.6 Hz, 6H), 2.40-2.24 (m, 2H) |
| I-769 | EL | D | 688.3 | 12.15 (br d, J = 7.4 Hz, 1H), 8.22-8.01 (m, 1H), 7.77-7.59 (m, |
| | | | | 1H), 7.46-7.33 (m, 2H), 7.29-7.21 (m, 1H), 7.18-7.11 (m, 1H), |
| | | | | 7.05 (t, J = 7.5 Hz, 1H), 6.99-6.86 (m, 2H), 6.45 (br s, 1H), 6.20- |
| | | | | 6.07 (m, 1H), 4.67-4.59 (m, 2H), 4.41-4.30 (m, 2H), 3.84 (br |
| | | | | s, 3H), 3.73 (d, J = 3.5 Hz, 3H), 3.67-3.59 (m, 2H), 3.30 (br s, |
| | | | | 1H), 3.13-3.02 (m, 6H), 2.38-2.24 (m, 2H) |
| I-770 | EM | D | 737.3 | |
| I-771 | EN | D | 698.3 | |
| I-772 | EU | D | 604.3 | |
| I-773 | EV | D | 614.4 | |
| I-774 | EO | D | 635.3 | 12.19-12.11 (m, 1H), 8.32 (d, J = 2.9 Hz, 1H), 8.13-8.07 (m, |
| | | | | 1H), 8.02 (d, J = 3.9 Hz, 1H), 7.72-7.65 (m, 1H), 7.43-7.37 (m, |
| | | | | 1H), 7.36-7.30 (m, 2H), 7.23 (dd, J = 4.5, 8.5 Hz, 1H), 7.16 (d, |
| | | | | J = 8.1 Hz, 1H), 7.05 (t, J = 7.3 Hz, 1H), 6.99-6.88 (m, 1H), 6.48- |
| | | | | 6.41 (m, 1H), 6.18-6.09 (m, 1H), 4.67-4.57 (m, 2H), 4.41-4.29 |
| | | | | (m, 2H), 3.83 (br s, 4H), 3.77-3.72 (m, 3H), 3.68-3.56 (m, 2H), |
| | | | | 3.27 (br s, 4H), 3.09 (q, J = 6.8 Hz, 2H), 2.37-2.25 (m, 2H) |
| I-775 | EP | D | 677.3 | |
| I-776 | EQ | D | 665.3 | |
| I-777 | ER | D | 655.3 | |
| I-778 | ES | D | 653.4 | |
| I-779 | ET | D | 669.3 | |

TABLE 13
Compounds synthesized via Method 3, coupling of
the corresponding amines and carboxylic acids

| | LCMS | |
|---|---|---|
| | (ESI+) | |
| | m/z | |
| I-# | (M + H)+ | ¹H NMR (400 MHz, DMSO-d₆) δ |
| I-1177 | 437.2 | 8.21 (s, 1H), 8.12 (s, 1H), 7.72-7.61 (m, 2H), 7.55 (s, 1H), 7.43-7.34 (m, 1H), |
| | | 6.28 (br s, 1H), 4.64 (t, J = 6.8 Hz, 2H), 4.40 (br d, J = 2.0 Hz, 2H), 4.01 (d, J = |
| | | 6.0 Hz, 2H), 3.60 (br d, J = 7.2 Hz, 2H), 3.26 (br s, 3H), 3.15 (br s, 2H), 3.03 (br |
| | | s, 3H), 2.38-2.35 (m, 3H), 2.34-2.25 (m, 2H) |
| I-1178 | 491.4 | 8.17 (s, 1H), 8.12 (d, J = 2.0 Hz, 1H), 7.69 (d, J = 2.8 Hz, 1H), 7.68-7.63 (m, |
| | | 1H), 7.50-7.43 (m, 1H), 7.42-7.34 (m, 1H), 6.27 (br s, 1H), 4.63 (t, J = 6.8 Hz, |
| | | 2H), 4.38 (br d, J = 12.4 Hz, 2H), 3.74 (s, 2H), 3.57 (s, 2H), 3.28-3.27 (m, 3H), |
| | | 3.10 (s, 2H), 3.03 (br s, 3H), 2.39 (br s, 4H), 2.26 (br d, J = 4.0 Hz, 2H), 1.51- |
| | | 1.44 (m, 4H), 1.37 (br d, J = 2.8 Hz, 2H) |
| I-1179 | 492.4 | 8.45-8.24 (m, 1H), 8.12 (s, 1H), 7.70 (s, 1H), 7.69-7.64 (m, 1H), 7.52-7.44 |
| | | (m, 1H), 7.42-7.37 (m, 1H), 6.41-6.23 (m, 1H), 4.64 (t, J = 6.8 Hz, 2H), 4.43- |
| | | 4.32 (m, 2H), 3.80 (br s, 3H), 3.57 (br s, 4H), 3.27 (br s, 4H), 3.18-3.08 (m, |
| | | 3H), 3.03 (br s, 3H), 2.87 (br s, 3H), 2.33 (br d, J = 1.6 Hz, 2H) |
| I-1180 | 478.3 | 8.29 (s, 1H), 8.12 (s, 1H), 7.70 (d, J = 2.8 Hz, 1H), 7.65 (br d, J = 17.2 Hz, 1H), |
| | | 7.41 (s, 1H), 7.40-7.36 (m, 1H), 6.31-6.24 (m, 1H), 4.63 (t, J = 6.8 Hz, 2H), |
| | | 4.38 (br s, 2H), 3.87 (br d, J = 3.2 Hz, 2H), 3.45 (br s, 1H), 3.26 (br s, 5H), 3.17- |
| | | 3.07 (m, 4H), 3.03 (br s, 3H), 2.95 (br s, 2H), 2.33 (br d, J = 1.6 Hz, 2H) |
| I-1181 | 652.4 | |
| I-1182 | 680.4 | |
| I-1183 | 642.3 | |
| I-1184 | 642.4 | |
| I-1185 | 669.3 | |

### Example I-373: Synthesis of 5-(1-(3-(1H-1,2,3-triazol-1-yl)propanoyl)-1,2,5,6-tetrahydropyridin-3-yl)-7-ethynyl-N,N-dimethylbenzofuran-2-carboxamide

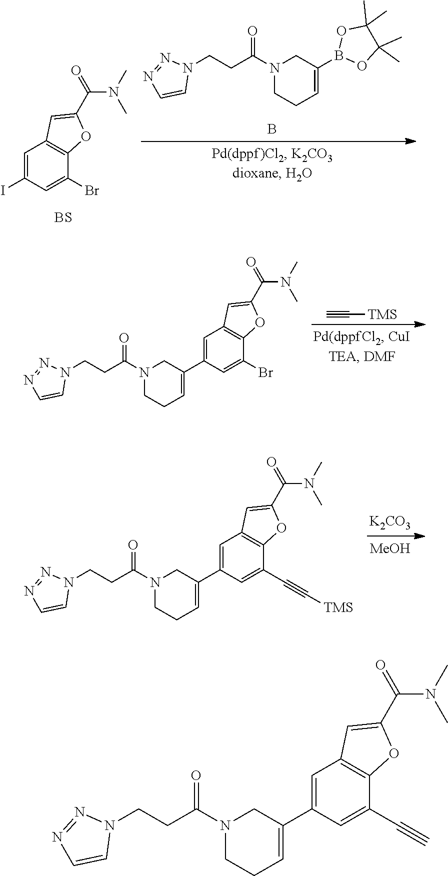

### Step 1—5-(1-(3-(1H-1,2,3-triazol-1-yl)propanoyl)-1,2,5,6-tetrahydropyridin-3-yl)-7-bromo-N,N-dimethylbenzofuran-2-carboxamide

To a solution of 7-bromo-5-iodo-N,N-dimethylbenzofuran-2-carboxamide (1.8 g, 4.6 mmol, Intermediate BS), 1-(5-(4,4,5,5-tetramethyl-1,3,2-dioxaborolan-2-yl)-3,6-dihydropyridin-1(2H)-yl)-3-(1H-1,2,3-triazol-1-yl)propan-1-one (1.97 g, 5.94 mmol, Intermediate B) in dioxane (20 mL) and H₂O (5 mL) was added Pd(dppf)Cl₂ (334 mg, 457 μmol) and K₂CO₃ (1.89 g, 13.7 mmol). The mixture was then stirred at 70° C. for 3 h under N₂. On completion, the reaction mixture was poured into ice water (25 mL) and extracted with ethyl acetate (25 mL×3). The combined organic layers were washed with brine (25 mL×3), dried over sodium sulfate, filtered and concentrated under reduced pressure to give a residue. The residue was purified by flash silica gel chromatography (ISCO®; 40 g SepaFlash® Silica Flash Column, Eluent of 0˜40% Ethyl acetate/Petroleum ether gradient @80 mL/min) to afford the title compound (800 mg, 37% yield) as a brown solid. LC-MS (ESI⁺) m/z 474.10 (M+H)⁺.

### Step 2—5-(1-(3-(1H-1,2,3-triazol-1-yl)propanoyl)-1,2,5,6-tetrahydropyridin-3-yl)-N,N-dimethyl-7-((trimethylsilyl)ethynyl)benzofuran-2-carboxamide

To a solution of 5-(1-(3-(1H-1,2,3-triazol-1-yl)propanoyl)-1,2,5,6-tetrahydropyridin-3-yl)-7-bromo-N,N-dimethylbenzofuran-2-carboxamide (200 mg, 400 μmol), ethynyl(trimethyl)silane (832 mg, 8.47 mmol, 1.17 mL, CAS #1066-54-2) in DMF (2 mL) was added Pd(dppf)Cl₂ (31.0 mg, 42.3 μmol) and CuI (8.06 mg, 42.3 μmol), and TEA (214 mg, 2.12 mmol, 295 μL). The mixture was then stirred at 60° C. for 10 h under N₂. On completion, the reaction mixture was poured into ice NH₄C1 (sat. aq. 5 mL) and extracted with ethyl acetate (5 mL×3). The combined organic layers were washed with brine (5 mL×3), dried over sodium sulfate, filtered and concentrated under reduced pressure to give a residue. The residue was purified by flash silica gel chromatography (ISCO®; 4 g SepaFlash® Silica Flash Column, Eluent of 0˜60% Ethyl acetate/Petroleum ether gradient @80 mL/min) to afford the title compound (160 mg, 77% yield) as a yellow solid. LC-MS (ESI⁺) m/z 490.3 (M+H)⁺.

### Step 3—5-(1-(3-(1H-1,2,3-triazol-1-yl)propanoyl)-1,2,5,6-tetrahydropyridin-3-yl)-7-ethynyl-N,N-dimethylbenzofuran-2-carboxamide

To a solution of 5-(1-(3-(1H-1,2,3-triazol-1-yl)propanoyl)-1,2,5,6-tetrahydropyridin-3-yl)-N,N-dimethyl-7-((trimethylsilyl)ethynyl)benzofuran-2-carboxamide (80 mg, 163 μmol) in MeOH (1 mL) was added K₂CO₃ (45.2 mg, 327 μmol). The mixture was then stirred at rt for 1 h. On completion, the residue was purified by prep-HPLC (column: Welch ultimate C18 150*25 mm*7 um; mobile phase: [water(FA)-ACN];B %: 20%-50%,15 min) to afford the title compound (25.39 mg, 37% yield) as a yellow solid. LC-MS (ESI⁺) m/z 418.10 (M+H)⁺; ¹H NMR (400 MHz, DMSO-d₆) δ 8.11 (s, 1H), 7.87-7.79 (m, 1H), 7.72-7.61 (m, 2H), 7.43 (d, J=7.6 Hz, 1H), 6.40-6.29 (m, 1H), 4.66-4.61 (m, 3H), 4.37 (br d, J=8.0 Hz, 2H), 3.59 (td, J=5.6, 16.4 Hz, 2H), 3.25 (br s, 3H), 3.16 (br t, J=6.8 Hz, 1H), 3.10 (br t, J=6.8 Hz, 1H), 3.03 (br s, 3H), 2.34-2.23 (m, 2H).

### Example I-374: Synthesis of 4-fluoro-5-(1-(2-formyl-1,2,3,4-tetrahydroisoquinoline-3-carbonyl)-1,2,5,6-tetrahydropyridin-3-yl)-N,N-dimethylbenzofuran-2-carboxamide

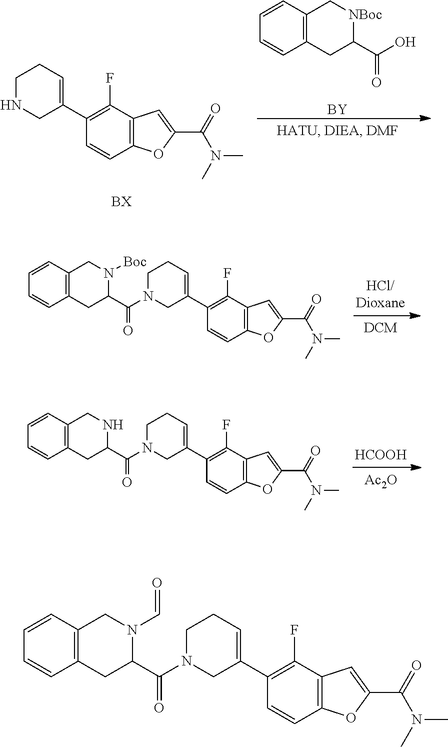

### Step 1—Tert-butyl 3-(5-(2-(dimethylcarbamoyl)-4-fluorobenzofuran-5-yl)-1,2,3,6-tetrahydropyridine-1-carbonyl)-3,4-dihydroisoquinoline-2(1H)-carboxylate

To a solution of 2-(tert-butoxycarbonyl)-1,2,3,4-tetrahydroisoquinoline-3-carboxylic acid (1.6 g, 5.8 mmol, CAS #151838-62-9, Intermediate BY) in DMF (20 mL) was added HATU (2.85 g, 7.50 mmol), DIEA (2.98 g, 23.1 mmol) and 4-fluoro-N,N-dimethyl-5-(1,2,5,6-tetrahydropyridin-3-yl)benzofuran-2-carboxamide (1.66 g, 5.77 mmol, Intermediate BX). The mixture was then stirred at 25° C. for 2 hrs. On completion, the reaction mixture was quenched with water (60 mL) and extracted with ethyl acetate (3×30 mL). The combined organic layers were washed with brine (2×30 mL) and dried over anhydrous sodium sulfate, filtered and the filtrate was concentrated in vacuo to give the crude residue. The residue was purified by column chromatography (SiO₂, Petroleum ether/Ethyl acetate=1/0 to 1/1) to give the title compound (3 g, 94% yield) as a yellow solid. LC-MS (ESI⁺) m/z 548.3 (M+H)⁺; ¹H NMR (400 MHz, DMSO-d₆) δ=7.96 (s, 1H), 7.61-7.36 (m, 3H), 7.28-7.12 (m, 4H), 6.12 (s, 1H), 4.69 (t, J=13.6 Hz, 1H), 4.46-4.29 (m, 2H), 3.34 (s, 1H), 3.25 (s, 3H), 3.07-3.00 (m, 3H), 2.89 (s, 3H), 2.74 (s, 3H), 1.46-1.28 (m, 9H).

### Step 2—4-Fluoro-N,N-dimethyl-5-(1-(1,2,3,4-tetrahydroisoquinoline-3-carbonyl)-1,2,5,6-tetrahydropyridin-3-yl)benzofuran-2-carboxamide

To a solution of tert-butyl 3-(5-(2-(dimethylcarbamoyl)-4-fluorobenzofuran-5-yl)-1,2,3,6-tetrahydropyridine-1-carbonyl)-3,4-dihydroisoquinoline-2(1H)-carboxylate (1 g, 2 mmol) in DCM (10 mL) was added HCl/dioxane (4 M, 2 mL). The mixture was then stirred at 25° C. for 2 hrs. On completion, the reaction mixture was concentrated in vacuo to give the title compound (1 g) as a yellow solid. LC-MS (ESI⁺) m/z 448.2 (M+H)⁺.

### Step 3—4-Fluoro-5-(1-(2-formyl-12,3,4-tetrahydroisoquinoline-3-carbonyl)-12,5,6-tetrahydropyridin-3-yl)-N,N-dimethylbenzofuran-2-carboxamide

To a solution of 4-fluoro-N,N-dimethyl-5-(1-(1,2,3,4-tetrahydroisoquinoline-3-carbonyl)-1,2,5,6-tetrahydropyridin-3-yl)benzofuran-2-carboxamide (1 g, 2 mmol) in Ac₂O (6 mL) and HCOOH (6 mL). The mixture was then stirred at 25° C. for 12 hrs. On completion, the reaction mixture was diluted with water (50 mL) and extracted with ethyl acetate (3×30 mL). The combined organic layers were washed with brine (2×30 mL) and dried over anhydrous sodium sulfate, filtered and the filtrate was concentrated in vacuo to give the crude residue. The crude residue was purified by reversed-phase HPLC (0.1% FA condition) to give the title compound (600 mg, 50% yield) as a white solid. LC-MS (ESI⁺) m/z 476.1 (M+H)⁺; ¹H NMR (400 MHz, DMSO-d₆) δ=8.26 (d, J=10.4 Hz, 1H), 7.54-7.48 (m, 1H), 7.46-7.39 (m, 2H), 7.21-7.15 (m, 4H), 6.13 (s, 1H), 5.39-5.27 (m, 1H), 4.92-4.69 (m, 1H), 4.53-4.45 (m, 1H), 4.43-4.23 (m, 2H), 3.77-3.66 (m, 2H), 3.15 (s, 6H), 3.05 (s, 2H), 2.43-2.32 (m, 2H).

### Example I-375 & I-376: Syntheses of ®-4-fluoro-5-(1-(2-formyl-1,2,3,4-tetrahydroisoquinoline-3-carbonyl)-1,2,5,6-tetrahydropyridin-3-yl)-N,N-dimethylbenzofuran-2-carboxamide (I-376) & (S)-4-fluoro-5-(1-(2-formyl-1,2,3,4-tetrahydroisoquinoline-3-carbonyl)-1,2,5,6-tetrahydropyridin-3-yl)-N,N-dimethylbenzofuran-2-carboxamide (I-375)

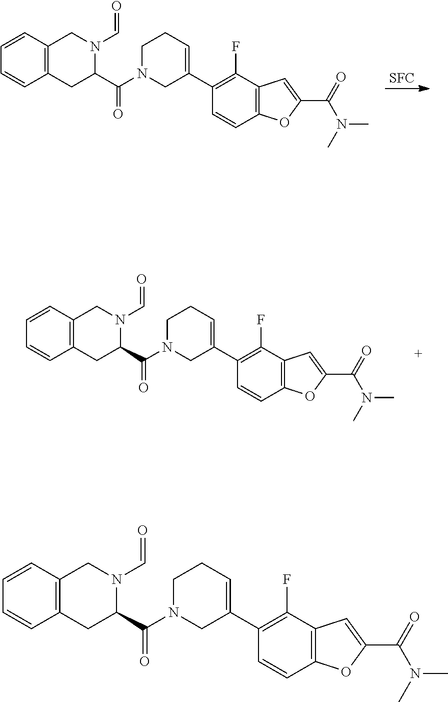

4-Fluoro-5-(1-(2-formyl-1,2,3,4-tetrahydroisoquinoline-3-carbonyl)-1,2,5,6-tetrahydropyridin-3-yl)-N,N-dimethylbenzofuran-2-carboxamide (300 mg, 0.63 mmol, Example I-374) was separated by SFC (column: DAICEL CHIRALCEL OJ (250 mm*30 mm,10 um);mobile phase: [CO2-MeOH];B %:25%, isocratic elution mode) to give the first eluting isomer (R)-4-fluoro-5-(1-(2-formyl-1,2,3,4-tetrahydroisoquinoline-3-carbonyl)-1,2,5,6-tetrahydropyridin-3-yl)-N,N-dimethylbenzofuran-2-carboxamide (100 mg, LC-MS (ESI⁺) m/z 476.1 (M+H)⁺; ¹H NMR (400 MHz, DMSO-d₆) δ=8.33-8.17 (m, 1H), 7.63-7.36 (m, 3H), 7.25-7.12 (m, 4H), 6.12 (s, 1H), 5.41-5.27 (m, 1H), 4.92-4.67 (m, 1H), 4.52-4.36 (m, 2H), 4.32-4.06 (m, 1H), 3.83-3.54 (m, 2H), 3.24 (s, 3H), 3.13-2.95 (m, 5H), 2.43-2.14 (m, 2H) and the second eluting isomer (S)-4-fluoro-5-(1-(2-formyl-1,2,3,4-tetrahydroisoquinoline-3-carbonyl)-1,2,5,6-tetrahydropyridin-3-yl)-N,N-dimethylbenzofuran-2-carboxamide (100 mg, LC-MS (ESI⁺) m/z 476.1 (M+H)⁺; ¹H NMR (400 MHz, DMSO-d₆) δ=8.33-8.13 (m, 1H), 7.64-7.35 (m, 3H), 7.29-7.08 (m, 4H), 6.12 (s, 1H), 5.43-5.27 (m, 1H), 4.92-4.67 (m, 1H), 4.51-4.36 (m, 2H), 4.33-4.06 (m, 1H), 3.84-3.51 (m, 2H), 3.24 (s, 3H), 3.14-2.94 (m, 5H), 2.46-2.20 (m, 2H)) as a white solids. The absolute stereochemistry of the enantiomers was assigned arbitrarily.

### Example I-377 (Method 4): Synthesis of 1-[5-[4-(5-ethyl-1-methyl-3,6-dihydro-2H-pyridin-4-yl)-7-fluoro-2-[4-(5-fluoro-3-methoxy-2-pyridyl)piperazine-1-carbonyl]-1H-indol-6-yl]-3,6-dihydro-2H-pyridin-1-yl]-3-(triazol-1-yl)propan-1-one (I-783)

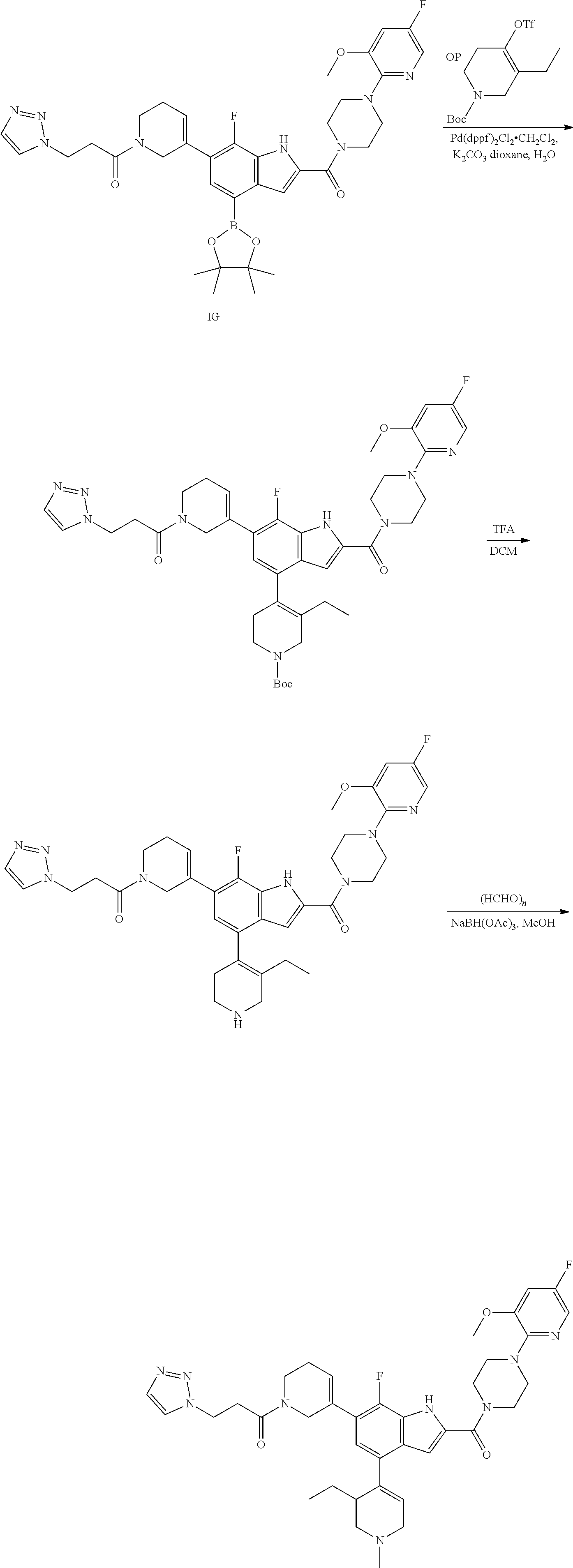

### Step 1—Tert-butyl 5-ethyl-4-[7-fluoro-2-[4-(5-fluoro-3-methoxy-2-pyridyl)piperazine-1-carbonyl]-6-[1-[3-(triazol-1-yl)propanoyl]-3,6-dihydro-2H-pyridin-5-yl]-1H-indol-4-yl]-3,6-dihydro-2H-pyridine-1-carboxylate

A mixture of tert-butyl 5-ethyl-4-(trifluoromethylsulfonyloxy)-3,6-dihydro-2H-pyridine-1-carboxylate (2.05 g, 5.69 mmol, Intermediate OP), 1-[5-[7-fluoro-2-[4-(5-fluoro-3-methoxy-2-pyridyl)piperazine-1-carbonyl]-4-(4,4,5,5-tetramethyl-1,3,2-dioxaborolan-2-yl)-1H-indol-6-yl]-3,6-dihydro-2H-pyridin-1-yl]-3-(triazol-1-yl)propan-1-one (200 mg, 285 μmol, Intermediate IG), Pd(dppf)Cl₂·CH₂Cl₂ (23 mg, 29 μmol) and K₂CO₃ (118 mg, 854 μmol) in dioxane (10 mL) and H₂O (2 mL) was degassed and purged with N₂ three times. Then the mixture was stirred at 80° C. for 1 hr under N₂ atmosphere. The reaction mixture was filtered and the filtrate was concentrated under reduced pressure to give a residue. The residue was purified by prep-HPLC (FA) to give the title compound (150 mg, 60% yield) as a brown solid. LC-MS (ESI⁺) m/z 786.6 (M+1)⁺.

### Step 2—1-[5-[4-(5-ethyl-1,2,3,6-tetrahydropyridin-4-yl)-7-fluoro-2-[4-(5-fluoro-3-methoxy-2-pyridyl)piperazine-1-carbonyl]-1H-indol-6-yl]-3,6-dihydro-2H-pyridin-1-yl]-3-(triazol-1-yl)propan-1-one

To a solution of tert-butyl 5-ethyl-4-[7-fluoro-2-[4-(5-fluoro-3-methoxy-2-pyridyl)piperazine-1-carbonyl]-6-[1-[3-(triazol-1-yl)propanoyl]-3,6-dihydro-2H-pyridin-5-yl]-1H-indol-4-yl]-3,6-dihydro-2H-pyridine-1-carboxylate (150 mg, 191 μmol) in DCM (0.5 mL) was added TFA (766 mg, 6.73 mmol, 0.50 mL). The mixture was then stirred at 20° C. for 0.5 hr. ON completion, the reaction mixture was concentrated under reduced pressure to give a residue. The residue was purified by SFC (NH₃H₂O condition) to give the title compound (20 mg, 15% yield,) as a white gum. LC-MS (ESI⁺) m/z 686.4 (M+1)⁺.

### Step 3—1-[5-[4-(5-Ethyl-1-methyl-3,6-dihydro-2H-pyridin-4-yl)-7-fluoro-2-[4-(5-fluoro-3-methoxy-2-pyridyl)piperazine-1-carbonyl]-1H-indol-6-yl]-3,6-dihydro-2H-pyridin-1-yl]-3-(triazol-1-yl)propan-1-one

To a solution of 1-[5-[4-(5-ethyl-1,2,3,6-tetrahydropyridin-4-yl)-7-fluoro-2-[4-(5-fluoro-3-methoxy-2-pyridyl)piperazine-1-carbonyl]-1H-indol-6-yl]-3,6-dihydro-2H-pyridin-1-yl]-3-(triazol-1-yl)propan-1-one (20 mg, 30 μmol) and NaBH(OAc)₃ (12 mg, 58 μmol) in MeOH (1 mL) was added (HCHO)n (20 mg). The mixture was then stirred at 20° C. for 1 hr. On completion, the mixture was filtered and the filtrate was concentrated under reduced pressure to give a residue. The residue was purified by prep-HPLC (FA condition) to give the title compound (20.93 mg, 97% yield) as a white solid. LC-MS (ESI⁺) m/z 700.3 (M+1)⁺; ¹H NMR (400 MHz, DMSO-d₆) δ=12.22-11.98 (m, 1H), 8.30-8.20 (m, 1H), 8.17-8.03 (m, 1H), 7.80 (d, J=2.4 Hz, 1H), 7.74-7.62 (m, 1H), 7.37 (dd, J=2.4, 10.4 Hz, 1H), 6.75-6.63 (m, 1H), 6.51 (br s, 1H), 6.13-6.02 (m, 1H), 4.62 (q, J=6.8 Hz, 2H), 4.38-4.22 (m, 2H), 3.85 (s, 3H), 3.80 (br s, 4H), 3.66-3.58 (m, 2H), 3.27 (br s, 4H), 3.09 (q, J=7.2 Hz, 2H), 2.96 (br s, 2H), 2.57 (brt, J=5.2 Hz, 2H), 2.34 (br s, 2H), 2.32 (s, 3H), 2.26 (br s, 2H), 1.79 (q, J=7.6 Hz, 2H), 0.82 (brt, J=7.2 Hz, 3H).

TABLE 14
Compounds synthesized via Method 4, coupling of the corresponding boronic
acid/esters and halogens in Step 1, followed by Steps 2 and 3.

| | | | LCMS | |
|---|---|---|---|---|
| | | | (ESI+) | |
| | | Boronic | m/z | |
| I-#^{a} | Halogen | Acid | (M + H)⁺ | ¹H NMR (400 MHz, DMSO-d₆) δ |
| I-780 | OF | OG | 674.4 | 12.04-12.00 (m, 1H), 8.13-8.10 (m, 1H), 7.81 (d, J = 2.4 |
| | | | | Hz, 1H), 7.71-7.67 (m, 1H), 7.38 (dd, J = 2.4, 10.4 Hz, |
| | | | | 1H), 7.03 (br s, 1H), 6.96-6.91 (m, 1H), 6.06 (br s, 1H), |
| | | | | 4.62 (d, J = 6.8 Hz, 2H), 4.34-4.28 (m, 2H), 3.86 (s, 3H), |
| | | | | 3.82 (br s, 3H), 3.66-3.58 (m, 2H), 3.29 (br s, 2H), 3.29- |
| | | | | 3.27 (m, 2H), 3.14 (br d, J = 6.8 Hz, 1H), 3.11-3.06 (m, |
| | | | | 2H), 2.98 (br d, J = 11.2 Hz, 1H), 2.33 (br d, J = 1.6 Hz, |
| | | | | 2H), 2.29-2.21 (m, 1H), 2.10-2.05 (m, 1H), 1.91 (s, 3H), |
| | | | | 1.76-1.72 (m, 1H), 1.68-1.61 (m, 4H), 1.42-1.33 (m, 1H) |
| I-781 | OF | OM | 702.3 | 12.15-12.01 (m, 1H), 8.15 (d, J = 12.8 Hz, 1H), 7.81 (d, J = |
| | | | | 2.4 Hz, 1H), 7.71-7.67 (m, 1H), 7.43-7.36 (m, 1H), 6.89 |
| | | | | (s, 1H), 6.70-6.62 (m, 1H), 6.06 (d, J = 1.6 Hz, 1H), 4.66- |
| | | | | 4.60 (m, 2H), 4.36-4.27 (m, 2H), 3.89-3.79 (m, 7H), 3.69- |
| | | | | 3.59 (m, 2H), 3.24-3.16 (m, 2H), 3.12-3.05 (m, 2H), |
| | | | | 3.00-2.88 (m, 2H), 2.38-2.33 (m, 2H), 2.28 (d, J = 9.6 Hz, |
| | | | | 1H), 2.24 (s, 3H), 2.20-2.03 (m, 2H), 1.86-1.75 (m, 1H), |
| | | | | 1.59-1.52 (m, 1H), 0.84-0.71 (m, 1H), 0.65-0.55 (m, 3H) |
| I-782 | OO | IG | 686.4 | 12.35-12.12 (m, 1H), 8.21 (s, 1H), 8.15-8.06 (m, 1H), |
| | | | | 7.80 (d, J = 2.4 Hz, 1H), 7.73-7.64 (m, 1H), 7.38 (dd, J = |
| | | | | 2.4, 10.4 Hz, 1H), 6.84-6.72 (m, 1H), 6.62 (br s, 1H), 6.16- |
| | | | | 6.04 (m, 1H), 4.66-4.59 (m, 2H), 4.35-4.27 (m, 2H), |
| | | | | 3.85 (s, 3H), 3.82 (br s, 6H), 3.64 (br d, J = 4.0 Hz, 2H), |
| | | | | 3.61-3.57 (m, 2H), 3.28 (br s, 4H), 3.12-3.06 (m, 2H), |
| | | | | 2.49 (d, J = 1.6 Hz, 3H), 2.37-2.25 (m, 2H), 2.19-2.06 (m, |
| | | | | 2H), 1.00-0.93 (m, 3H) |
| I-784 | OF | OG | 688.6 | 12.01 (s, 1H), 8.15-8.06 (m, 1H), 7.80 (d, J = 2.4 Hz, 1H), |
| | | | | 7.72-7.64 (m, 1H), 7.38 (dd, J = 2.4, 10.4 Hz, 1H), 7.13- |
| | | | | 7.03 (m, 1H), 6.96-6.90 (m, 1H), 6.06 (s, 1H), 4.62 (q, J = |
| | | | | 6.8 Hz, 2H), 4.40-4.22 (m, 2H), 3.86 (s, 3H), 3.82 (s, 4H), |
| | | | | 3.68-3.55 (m, 2H), 3.42-3.36 (m, 1H), 3.29 (s, 2H), 3.19- |
| | | | | 3.02 (m, 4H), 2.43 (dd, J = 7.6, 12.8 Hz, 1H), 2.33 (td, J = |
| | | | | 2.0, 4.0 Hz, 2H), 2.26 (d, J = 2.4 Hz, 1H), 2.10-1.99 (m, |
| | | | | 1H), 1.98-1.87 (m, 1H), 1.77-1.63 (m, 4H), 1.62-1.53 |
| | | | | (m, 1H), 1.45-1.32 (m, 1H), 0.83 (td, J = 3.6, 6.8 Hz, 3H) |
| I-785 | PS | B | 646.3 | 12.10 (s, 1H), 8.21-7.96 (m, 1H), 7.86-7.77 (m, 1H), 7.74- |
| | | | | 7.62 (m, 1H), 7.48-7.30 (m, 1H), 6.96-6.79 (m, 2H), |
| | | | | 6.08 (s, 1H), 4.67-4.58 (m, 2H), 4.37-4.25 (m, 2H), 4.01- |
| | | | | 3.94 (m, 1H), 3.91-3.73 (m, 10H), 3.71-3.55 (m, 3H), |
| | | | | 3.22-3.02 (m, 4H), 2.38-2.24 (m, 5H), 2.04-1.85 (m, 2H) |

TABLE 15
Compounds synthesized via Method 4, coupling of the corresponding boronic
acid/esters and halogens in Step 1, followed by Steps 2 and 3.

| | | | LCMS | |
|---|---|---|---|---|
| | | | (ESI+) | |
| | | Boronic | m/z | |
| I-# | Halogen | Acid | (M + H)⁺ | ¹H NMR (400 MHz, DMSO-d₆) δ |
| I-1221^{b} | UW | UX | 694.4 | 11.64-10.66 (m, 1H), 8.73-8.54 (m, 1H), 7.88 (dd, J = |
| | | | | 8.4, 18.8 Hz, 1H), 7.82 (s, 1H), 7.78 (d, J = 2.4 Hz, |
| | | | | 1H), 7.42-7.32 (m, 1H), 7.13-7.06 (m, 1H), 6.93- |
| | | | | 6.69 (m, 1H), 6.14-5.98 (m, 1H), 4.55-4.37 (m, |
| | | | | 1H), 4.16-4.09 (m, 1H), 4.07-3.98 (m, 2H), 3.94 (s, |
| | | | | 2H), 3.83 (s, 3H), 3.70 (s, 2H), 3.67-3.57 (m, 4H), |
| | | | | 3.51-3.39 (m, 2H), 3.29-3.19 (m, 4H), 2.91-2.86 |
| | | | | (m, 3H), 2.85-2.78 (m, 1H), 2.74-2.67 (m, 1H), |
| | | | | 2.64-2.61 (m, 1H), 2.59 (dd, J = 4.0, 7.2 Hz, 3H), |
| | | | | 2.43-2.27 (m, 2H), 2.14-2.07 (m, 3H), 1.44-1.15 |
| | | | | (m, 2H), 1.03 (t, J = 7.6 Hz, 3H) |

^{a}Step 1 coupling of boronic acids/esters with bromide, chloride, and triflates under standard conditions. Step 2 TFA in DCM was run from 0-25° C. for 1-2 h. Step 3 methylation with HCHO and NaBH(OAc)₃ in THF or MeOH at rt for 0.5-2 hr. The final compounds were purified by reverse phase HPLC, prep-HPLC, or SFC.
^{b}Instead of cross coupling in Step 1, an amine and bromide were coupled using NaH in DMF for 0-40 c. for 12 hrs. Step 2 used HCl/Dioxane at rt for 0.5 hr for the deprotection. Step 3 used NaBH₃CN with formaldehyde in MeOH at rt for 1 hr for the reductive amination.

TABLE 16
Compounds synthesized via Method 5, coupling of the corresponding amines
and acyl chlorides in Step 1, followed by hydrolysis in Steps 2.

| | | | LCMS | |
|---|---|---|---|---|
| | | | (ESI+) | |
| | | Acyl | m/z | |
| I-# | Amine | Chloride | (M + H)⁺ | ¹H NMR (400 MHz, DMSO-d₆) δ |
| I-1218^{b} | VH | TC | 595.3 | 7.84 (s, 1H), 7.69 (d, J = 7.6 Hz, 1H), 7.29-7.12 (m, 3H), |
| | | | | 6.16-6.02 (m, 1H), 4.78 (s, 2H), 4.62-4.51 (m, 2H), 4.43- |
| | | | | 4.28 (m, 1H), 4.17-4.05 (m, 2H), 4.00 (q, J = 7.2 Hz, 1H), |
| | | | | 3.73-3.60 (m, 1H), 3.58-3.36 (m, 3H), 2.55 (s, 3H), 2.48- |
| | | | | 2.37 (m, 3H), 2.35-2.24 (m, 1H), 2.10-2.04 (m, 3H), 1.38- |
| | | | | 1.23 (m, 3H), 1.05 (q, J = 7.2 Hz, 3H), 0.54-0.43 (m, 1H), |
| | | | | 0.20-0.08 (m, 2H), −0.22 (d, J = 4.0 Hz, 2H) |
| I-1223 | VG | QX | 609.3 | 7.90-7.83 (m, 1H), 7.74-7.69 (m, 1H), 7.28-7.14 (m, |
| | | | | 2H), 7.13-7.09 (m, 1H), 6.77-6.61 (m, 1H), 6.08-6.02 |
| | | | | (m, 1H), 4.80-4.72 (m, 2H), 4.62-4.56 (m, 2H), 4.45- |
| | | | | 4.32 (m, 1H), 4.12-4.01 (m, 1H), 3.79 (s, 2H), 3.70 (s, 2H), |
| | | | | 3.66-3.57 (m, 3H), 3.54-3.50 (m, 2H), 2.80 (q, J = 7.2 Hz, |
| | | | | 2H), 2.33 (d, J = 1.6 Hz, 2H), 2.30-2.23 (m, 1H), 2.21 (d, J = |
| | | | | 3.6 Hz, 3H), 1.26 (t, J = 7.6 Hz, 3H), 0.27-0.18 (m, 6H) |
| I-1217^{b} | UG | TC | 669.3 | 7.78 (d, J = 2.4 Hz, 1H), 7.69 (d, J = 8.0 Hz, 1H), 7.66 (s, |
| | | | | 1H), 7.36 (dd, J = 2.4, 10.4 Hz, 1H), 7.22 (d, J = 8.0 Hz, |
| | | | | 1H), 7.00 (s, 1H), 6.13-6.04 (m, 1H), 4.42-4.25 (m, 1H), |
| | | | | 4.16-4.05 (m, 1H), 3.83 (s, 3H), 3.70-3.60 (m, 4H), 3.59- |
| | | | | 3.48 (m, 2H), 3.46-3.40 (m, 1H), 3.24 (br s, 4H), 2.55 (s, |
| | | | | 3H), 2.48-2.34 (m, 4H), 2.27 (br d, J = 3.6 Hz, 1H), 2.08 (s, |
| | | | | 2H), 2.05 (s, 1H), 1.04 (t, J = 7.2 Hz, 3H), 0.52-0.44 (m, |
| | | | | 1H), 0.20-0.08 (m, 2H), −0.17-−0.25 (m, 2H) |
| I-1222 | UU | RW | 697.4 | 7.78 (d, J = 2.1 Hz, 1H), 7.74-7.67 (m, 2H), 7.35 (dd, J = |
| | | | | 2.4, 10.4 Hz, 1H), 7.24 (d, J = 7.6 Hz, 1H), 6.96 (s, 1H), |
| | | | | 6.78-6.60 (m, 1H), 6.04 (s, 1H), 4.37 (d, J = 19.2 Hz, 1H), |
| | | | | 4.15-4.02 (m, 1H), 3.83 (s, 3H), 3.72-3.54 (m, 6H), 3.50- |
| | | | | 3.42 (m, 3H), 3.25 (s, 5H), 2.80 (q, J = 7.2 Hz, 2H), 2.40- |
| | | | | 2.26 (m, 2H), 2.20 (s, 3H), 1.26 (t, J = 7.2 Hz, 4H), 0.29- |
| | | | | 0.15 (m, 6H) |
| I-1216 | UG | RW | 713.5 | 13.55-11.80 (m, 1H), 7.78 (d, J = 2.4 Hz, 1H), 7.71-7.67 |
| | | | | (m, 1H), 7.66 (d, J = 1.6 Hz, 1H), 7.39-7.33 (m, 1H), 7.22 |
| | | | | (d, J = 7.6 Hz, 1H), 7.00 (d, J = 1.6 Hz, 1H), 6.16-6.06 (m, |
| | | | | 1H), 4.44-4.25 (m, 1H), 4.15-4.06 (m, 1H), 3.83 (s, 3H), |
| | | | | 3.76-3.65 (m, 3H), 3.64-3.58 (m, 2H), 3.57-3.49 (m, |
| | | | | 3H), 3.47-3.41 (m, 2H), 3.24 ( s, 4H), 2.55 (s, 3H), 2.47- |
| | | | | 2.40 (m, 2H), 2.39-2.28 (m, 2H), 1.04 (t, J = 7.6 Hz, 3H), |
| | | | | 0.53-0.44 (m, 1H), 0.18-0.07 (m, 2H), −0.18-−0.25 (m, 2H) |

^{a}Step 1 acylation was run for 2 hrs from 0-25° C. under standard conditions. Step 2 hydrolysis was run 0.5-2 hr at rt. Hydrolysis could also be achieved with NaOH in THF and H₂O. Purification of final compounds under standard techniques such as column chromatography and reverse-phase HPLC under a variety of conditions.
^{b}Only acylation, no hydrolysis step performed.

TABLE 17
Compounds synthesized via Method 5, coupling of the amines and
acyl chlorides in Step 1, followed by hydrolysis in Step 2.

| | LCMS | |
|---|---|---|
| | (ESI+) | |
| | m/z | |
| I-# | (M + H)+ | ¹H NMR (400 MHz, DMSO-d₆) δ |
| I-1241 | 701.3 | 7.78 (d, J = 2.4 Hz, 1H), 7.67 (t, J = 8.0 Hz, 1H), 7.61 (d, J = 1.6 Hz, 1H), 7.35 (dd, |
| | | J = 2.4, 10.4 Hz, 1H), 7.22 (d, J = 7.6 Hz, 1H), 6.92 (d, J = 6.0 Hz, 1H), 4.45-4.27 |
| | | (m, 1H), 3.83 (s, 3H), 3.63 (d, J = 10.4 Hz, 4H), 3.24 (s, 4H), 3.03-2.84 (m, 3H), |
| | | 2.80-2.70 (m, 1H), 2.55 (s, 2H), 2.54 (s, 3H), 2.39-2.32 (m, 2H), 2.15-2.01 (m, |
| | | 2H), 1.86-1.78 (m, 1H), 1.59-1.49 (m, 1H), 1.03-0.97 (m, 3H), 0.66-0.58 (m, |
| | | 1H), 0.27-0.13 (m, 2H), −0.16 (dt, J = 4.4, 8.4 Hz, 2H) |

^{a}Step 1 coupling of boronic acids/esters with bromide, chloride, and triflates under standard conditions. Step 2 TFA in DCM was run from 0-25° C. for 1-2 h. Step 3 methylation with HCHO and NaBH(Oac)₃ in THF or MeOH at rt for 0.5-2 hr. The final compounds were purified by reverse phase HPLC, prep-HPLC, or SFC.

### Example I-378 (Method 5): Synthesis of 3-(3-(1—(Cyclopropylmethyl)-7-(2-ethyl-6-methylpyridin-3-yl)-5-(4-(5-fluoro-3-methoxypyridin-2-yl)piperazine-1-carbonyl)-1H-indol-2-yl)-5,6-dihydropyridin-1(2H)-yl)-3-oxopropanoic acid (I-789)

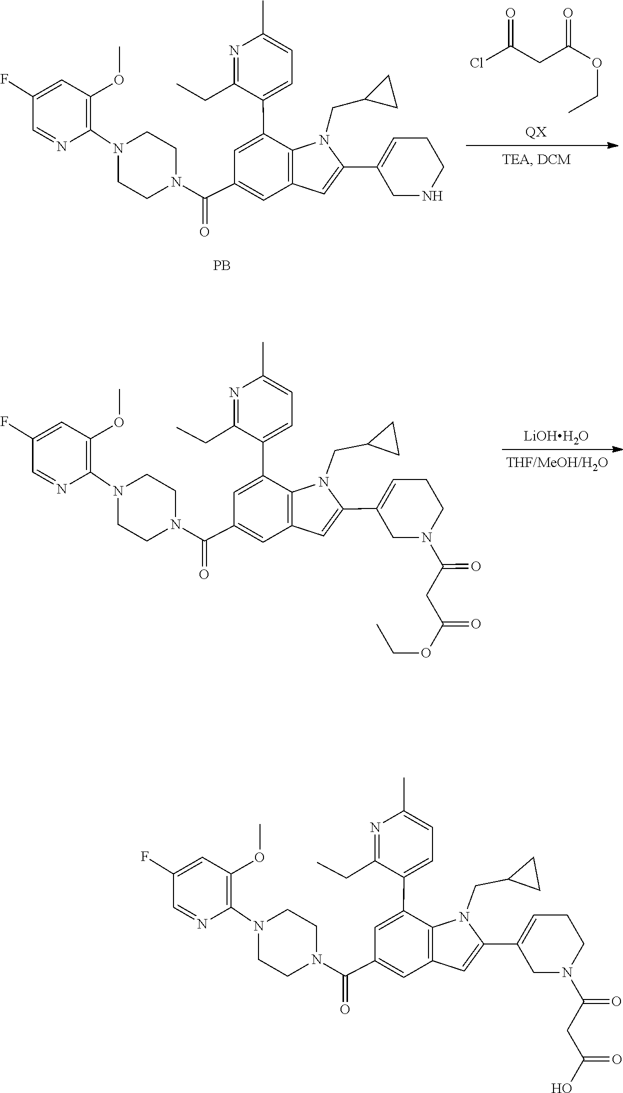

### Step 1—Ethyl 3-(3-(1-(cyclopropylmethyl-7-(2-ethyl-6-methylpyridin-3-yl)-5-(4-(5-fluoro-3-methoxypyridin-2-yl)piperazine-1-carbonyl)-1H-indol-2-yl)-5,6-dihydropyridin-1(2H)-yl)-3-oxopropanoate

To a solution of (1-(cyclopropylmethyl)-7-(2-ethyl-6-methylpyridin-3-yl)-2-(1,2,5,6-tetrahydropyridin-3-yl)-1H-indol-5-yl)(4-(5-fluoro-3-methoxypyridin-2-yl)piperazin-1-yl)methanone (120 mg, 197 μmol, Intermediate PB) in DCM (4 mL) was added TEA (59.8 mg, 591 μmol, 82.31 μL) and ethyl 3-chloro-3-oxopropanoate (35.6 mg, 237 μmol, 29.78 μL, CAS #36239-09-5, Intermediate QX). The mixture was then stirred at 25° C. for 2 hrs. On completion, the reaction mixture was concentrated in vacuo to give the crude residue. The residue was purified by column chromatography (SiO₂, Petroleum ether: Ethyl acetate=1/1 to 0/1) to give the title compound (55 mg, 31% yield) as a white solid. LC-MS (ESI+) m/z 723.4 (M+H)⁺.

### Step 2—3-(3-(1—(Cyclopropylmethyl)-7-(2-ethyl-6-methylpyridin-3-yl)-5-(4-(5-fluoro-3-methoxypyridin-2-yl)piperazine-1-carbonyl)-1H-indol-2-yl)-5,6-dihydropyridin-1(2H)-yl)-3-oxopropanoic acid

To a solution of ethyl 3-(3-(1-(cyclopropylmethyl)-7-(2-ethyl-6-methylpyridin-3-yl)-5-(4-(5-fluoro-3-methoxypyridin-2-yl)piperazine-1-carbonyl)-1H-indol-2-yl)-5,6-dihydropyridin-1(2H)-yl)-3-oxopropanoate (55 mg, 76 μmol) in THF (4 mL), MeOH (1 mL) and H₂O (1 mL) was added LiOH·H₂O (9.58 mg, 228 μmol). The mixture was then stirred at 25° C. for 2 hrs. On completion, HCl was added to the mixture until the pH was 6.0 and then concentrated in vacuo to give the crude residue. The residue was purified by reverse-phase HPLC (0.1% FA condition) to give the title compound (30 mg, 52% yield) as a white solid. LC-MS (ESI+) m/z 695.3 (M+H)⁺; ¹H NMR (400 MHz, DMSO-d6) δ=7.78 (d, J=2.4 Hz, 1H), 7.71-7.62 (m, 2H), 7.36 (dd, J=2.4, 10.4 Hz, 1H), 7.21 (d, =7.6 Hz, 1H), 6.91 (d, =1.2 Hz, 1H), 6.79-6.64 (m, 1H), 6.06-5.95 (m, 1H), 4.40-4.28 (m, 1H), 4.14-4.05 (m, 1H), 3.83 (s, 3), 3.64 (br d, J=14.4 Hz, 4H), 3.49 (br s, 1H), 3.47 (br s, 2H), 3.42 (br d, J=5.6 Hz, 2H), 3.39 (br s, 2H), 3.24 (br s, 4H), 2.55 (s, 3H), 2.46-2.42 (m, 2H), 2.36-2.23 (m, 2H), 1.03 (t, J=7.6 Hz, 3H), 0.57-0.42 (m, 1H), 0.14-0.03 (m, 2H), −0.18-−0.29 (m, 2H).

TABLE 18
Compounds synthesized via Method 5, coupling of the corresponding amines
and acyl chlorides in Step 1, followed by hydrolysis in Steps 2.

| | | | LCMS | |
|---|---|---|---|---|
| | | | (ESI+) | |
| | | Acyl | m/z | |
| I-#^{a} | Amine | Chloride | (M + H)⁺ | ¹H NMR (400 MHz, DMSO-d₆) δ |
| I-786 | ON | QX | 645.5 | 12.52-12.16 (m, 1H), 8.53 (d, J = 5.2 Hz, 1H), 8.42 (s, |
| | | | | 1H), 7.78 (d, J = 2.4 Hz, 1H), 7.43 (d, J = 5.2 Hz, 1H), |
| | | | | 7.36 (dd, J = 2.4, 10.4 Hz, 1H), 7.00-6.87 (m, 1H), 6.35 |
| | | | | (d, J = 2.8 Hz, 1H), 6.17 (s, 1H), 4.43-4.30 (m, 2H), |
| | | | | 3.83 (s, 3H), 3.76 (s, 4H), 3.68-3.55 (m, 2H), 3.48 (s, |
| | | | | 2H), 3.23 (s, 6H), 2.40-2.25 (m, 2H), 1.05-0.96 (m, 3H) |
| I-787 | RU | QX | 571.3 | 12.35 (s, 1H), 8.55 (d, J = 5.2 Hz, 1H), 8.49-8.32 (m, |
| | | | | 1H), 7.45 (s, 1H), 7.35-7.17 (m, 1H), 6.90 (d, J = 4.4 |
| | | | | Hz, 1H), 6.78-6.57 (m, 1H), 6.29-6.11 (m, 1H), 4.99 |
| | | | | (s, 1H), 4.79 (s, 2H), 4.60 (s, 1H), 4.47-4.27 (m, 2H), |
| | | | | 4.20-3.94 (m, 2H), 3.69-3.54 (m, 2H), 3.47 (s, 2H), |
| | | | | 2.39-2.24 (m, 4H), 1.38-1.27 (m, 3H), 1.04 (td, J = |
| | | | | 7.6, 14.4 Hz, 3H) |
| I-788 | PH | QX | 585.3 | 12.31 (s, 1H), 8.53-8.16 (m, 1H), 7.46-7.13 (m, 2H), |
| | | | | 6.86 (s, 1H), 6.77-6.61 (m, 1H), 6.18 (d, J = 2.4 Hz, |
| | | | | 1H), 5.00 (d, J = 1.6 Hz, 1H), 4.79 (s, 2H), 4.60 (s, 1H), |
| | | | | 4.42-4.27 (m, 2H), 4.19-3.96 (m, 2H), 3.67-3.56 (m, |
| | | | | 2H), 3.46 (s, 2H), 2.53 (s, 4H), 2.41-2.24 (m, 3H), 1.39- |
| | | | | 1.26 (m, 3H), 1.12-0.93 (m, 3H) |
| I-790 | TU | TD | 631.2 | 12.36 (s, 1H), 8.54 (d, J = 5.2 Hz, 1H), 8.48-8.31 (m, |
| | | | | 1H), 7.78 (d, J = 2.4 Hz, 1H), 7.43 (d, J = 5.2 Hz, 1H), |
| | | | | 7.36 (dd, J = 2.4, 10.4 Hz, 1H), 6.97-6.83 (m, 1H), 6.36 |
| | | | | (s, 1H), 6.23-6.10 (m, 1H), 4.47-4.27 (m, 2H), 3.83 (s, |
| | | | | 3H), 3.76 (s, 4H), 3.68 (t, J = 5.6 Hz, 1H), 3.56 (t, J = |
| | | | | 5.6 Hz, 2H), 3.23 (s, 5H), 2.42-2.25 (m, 2H), 1.01 (t, J = |
| | | | | 7.6 Hz, 3H) |
| I-791 | RY | RW | 595.3 | 8.30 (br d, J = 3.6 Hz, 1H), 7.87-7.81 (m, 2H), 7.27- |
| | | | | 7.15 (m, 2H), 7.11 (br s, 1H), 6.69-6.62 (m, 1H), 5.99 |
| | | | | (br s, 1H), 4.76 (br d, J = 14.4 Hz, 2H), 4.59 (br s, 2H), |
| | | | | 4.43-4.04 (m, 3H), 3.83 (s, 3H), 3.79 (br s, 1H), 3.70 |
| | | | | (br s, 2H), 3.61-3.47 (m, 4H), 3.06 (br s, 2H), 2.35 (br |
| | | | | s, 1H), 2.23 (br d, J = 2.0 Hz, 1H), 0.57-0.50 (m, 1H), |
| | | | | 0.10 (br d, J = 7.2 Hz, 2H), −0.23 (br s, 2H) |
| I-792 | RJ | RW | 709.4 | 7.71-7.64 (m, 2H), 7.28 (d, J = 10.4 Hz, 1H), 7.21 (d, J = |
| | | | | 8.0 Hz, 1H), 6.91 (s, 1H), 6.79-6.64 (m, 1H), 5.99 (s, |
| | | | | 1H), 4.35 (d, J = 17.6 Hz, 1H), 4.10 (s, 1H), 3.79 (s, 3H), |
| | | | | 3.71-3.50 (m, 7H), 3.44-3.39 (m, 4H), 3.23 (s, 4H), |
| | | | | 2.55 (s, 3H), 2.47-2.39 (m, 2H), 2.33 (d, J = 2.0 Hz, |
| | | | | 1H), 2.24 (d, J = 2.8 Hz, 3H), 1.03 (t, J = 7.2 Hz, 3H), |
| | | | | 0.50 (d, J = 6.0 Hz, 1H), 0.09 (dd, J = 5.2, 8.0 Hz, |
| | | | | 2H), −0.17-−0.27 (m, 2H) |
| I-793 ^{b} | QV | TC | 651.4 | 7.78 (d, J = 2.0 Hz, 1H), 7.73-7.67 (m, 2H), 7.35 (d, J = |
| | | | | 10.0 Hz, 1H), 7.23 (d, J = 8.0 Hz, 1H), 6.94 (s, 1H), 6.80- |
| | | | | 6.65 (m, 1H), 6.09-6.01 (m, 1H), 4.39-4.26 (m, 1H), |
| | | | | 4.16-4.04 (m, 1H), 3.84 (s, 3H), 3.70-3.63 (m, 4H), |
| | | | | 3.61-3.53 (m, 2H), 3.25 (s, 4H), 2.81 (q, J = 7.2 Hz, |
| | | | | 2H), 2.34 (dd, J = 2.4, 5.2 Hz, 2H), 2.24 (d, J = 4.4 Hz, |
| | | | | 2H), 2.20 (s, 3H), 2.08 (d, J = 4.8 Hz, 3H), 1.28 (t, J = |
| | | | | 7.6 Hz, 3H), 0.47 (dd, J = 2.4, 8.0 Hz, 1H), 0.13-0.05 |
| | | | | (m, 2H), −0.27 (d, J = 4.0 Hz, 2H) |
| I-794 | TO | RW | 669.4 | 7.78 (d, J = 2.4 Hz, 1H), 7.71-7.66 (m, 2H), 7.35 (dd, J = |
| | | | | 2.4, 10.4 Hz, 1H), 7.20 (d, J = 7.6 Hz, 1H), 6.89 (s, |
| | | | | 1H), 6.72-6.62 (m, 1H), 6.05 (d, J = 8.0 Hz, 1H), 4.33- |
| | | | | 4.22 (m, 1H), 4.17-4.09 (m, 1H), 3.83 (s, 3H), 3.82- |
| | | | | 3.71 (m, 2H), 3.65 (s, 4H), 3.60-3.53 (m, 2H), 3.50 (d, J = |
| | | | | 3.6 Hz, 2H), 3.47-3.41 (m, 2H), 3.23 (s, 2H), 2.54 (s, |
| | | | | 3H), 2.47-2.40 (m, 2H), 2.37-2.25 (m, 2H), 1.04 (t, J = |
| | | | | 7.6 Hz, 3H), 0.64 (q, J = 6.4 Hz, 3H) |
| I-795 ^{b} | RY | RW | 609.3 | 8.31 (br d, J = 3.6 Hz, 1H), 7.88-7.82 (m, 2H), 7.27- |
| | | | | 7.14 (m, 2H), 7.12 (br s, 1H), 6.77-6.63 (m, 1H), 6.08- |
| | | | | 6.00 (m, 1H), 4.76 (br d, J = 9.6 Hz, 2H), 4.59 (br s, 2H), |
| | | | | 4.29 (br t, J = 18.4 Hz, 1H), 4.19-4.11 (m, 1H), 3.83 (br |
| | | | | s, 3H), 3.80 (br s, 1H), 3.70 (br s, 3H), 3.66-3.63 (m, |
| | | | | 4H), 3.60-3.47 (m, 4H), 2.37 (br s, 2H), 0.58-0.49 (m, |
| | | | | 1H), 0.10 (br d, J = 7.6 Hz, 2H), −0.23 (br s, 2H) |
| I-796 | TF | TD | 667.3 | 8.70-8.46 (m, 2H), 7.78 (d, J = 2.4 Hz, 1H), 7.73 (s, |
| | | | | 1H), 7.45 (d, J = 4.4 Hz, 1H), 7.36 (dd, J = 2.4, 10.4 Hz, |
| | | | | 1H), 6.95 (s, 1H), 6.79-6.62 (m, 1H), 6.15-5.94 (m, |
| | | | | 1H), 4.46-4.21 (m, 1H), 4.18-4.02 (m, 1H), 3.83 (s, |
| | | | | 3H), 3.74-3.61 (m, 4H), 3.57-3.51 (m, 2H), 3.43-3.39 |
| | | | | (m, 2H), 3.36-3.31 (m, 4H), 2.48-2.26 (m, 4H), 0.96 |
| | | | | (t, J = 7.6 Hz, 3H), 0.59-0.43 (m, 1H), 0.18-0.02 (m, |
| | | | | 2H), −0.16-−0.30 (m, 2H) |
| I-797 | RA | QY | 667.5 | 8.63 (dd, J = 1.6, 4.8 Hz, 1H), 7.85-7.80 (m, 1H), 7.78 |
| | | | | (d, J = 2.4 Hz, 1H), 7.72 (d, J = 1.6 Hz, 1H), 7.39-7.33 |
| | | | | (m, 2H), 6.96 (d, J = 1.6 Hz, 1H), 6.76-6.60 (m, 1H), |
| | | | | 6.09-5.97 (m, 1H), 4.38-4.22 (m, 1H), 4.15-4.04 (m, |
| | | | | 1H), 3.83 (s, 3H), 3.71-3.62 (m, 4H), 3.56-3.50 (m, |
| | | | | 2H), 3.24 (br s, 6H), 2.56-2.53 (m, 1H), 2.47-2.41 (m, |
| | | | | 1H), 2.34 (br dd, J = 2.4, 8.4 Hz, 2H), 1.09-1.03 (m, |
| | | | | 3H), 0.53-0.44 (m, 1H), 0.14-0.05 (m, 2H), −0.20-−0.28 |
| | | | | (m, 2H) |
| I-798 | TH | TD | 607.5 | 7.88 (d, J = 5.6 Hz, 1H), 7.73 (d, J = 7.6 Hz, 1H), 7.35- |
| | | | | 7.15 (m, 2H), 7.12 (s, 1H), 6.80-6.56 (m, 1H), 6.17- |
| | | | | 5.98 (m, 1H), 4.78 (d, J = 10.0 Hz, 2H), 4.58 (s, 2H), |
| | | | | 4.44-3.91 (m, 4H), 3.81-3.49 (m, 4H), 2.81 (q, J = 7.6 |
| | | | | Hz, 2H), 2.42-2.30 (m, 2H), 2.28-2.14 (m, 3H), 1.40- |
| | | | | 1.20 (m, 6H), 0.48 (d, J = 5.6 Hz, 1H), 0.24-0.00 (m, |
| | | | | 2H), −0.26 (s, 2H) |
| I-799 ^{b} | TH | TD | 635.4 | 7.88 (d, J = 5.2 Hz, 1H), 7.76-7.64 (m, 1H), 7.41-7.14 |
| | | | | (m, 2H), 7.12 (d, J = 1.6 Hz, 1H), 6.79-6.57 (m, 1H), |
| | | | | 6.07 (s, 1H), 4.78 (d, J = 9.6 Hz, 2H), 4.58 (s, 2H), 4.41- |
| | | | | 3.96 (m, 6H), 3.79-3.42 (m, 3H), 3.28-3.15 (m, 1H), |
| | | | | 2.81 (q, J = 7.6 Hz, 2H), 2.41-2.32 (m, 2H), 2.28-2.10 |
| | | | | (m, 3H), 1.40-1.27 (m, 6H), 1.26-1.02 (m, 3H), 0.48 |
| | | | | (d, J = 4.4 Hz, 1H), 0.23-−0.03 (m, 2H), −0.16-−0.38 |
| | | | | (m, 2H) |
| I-800 | PA | RW | 697.3 | 7.78 (d, J = 2.4 Hz, 1H), 7.71-7.65 (m, 2H), 7.35 (dd, J = |
| | | | | 2.4, 10.4 Hz, 1H), 7.22 (d, J = 7.6 Hz, 1H), 6.93 (d, J = |
| | | | | 1.6 Hz, 1H), 6.78-6.57 (m, 1H), 6.05-5.97 (m, 1H), |
| | | | | 4.43-4.31 (m, 1H), 4.10-4.00 (m, 1H), 3.83 (s, 3H), |
| | | | | 3.76-3.53 (m, 7H), 3.53-3.49 (m, 2H), 3.24 (s, 8H), |
| | | | | 2.47-2.42 (m, 1H), 2.39 (d, J = 14.8 Hz, 2H), 2.34- |
| | | | | 2.25 (m, 1H), 1.38-1.28 (m, 1H), 1.03 (t, J = 7.6 Hz, |
| | | | | 3H), 0.28-0.19 (m, 6H) |
| I-801 | TN | RW | 623.3 | 12.92-12.46 (m, 1H), 7.99-7.78 (m, 1H), 7.71 (d, J = |
| | | | | 8.0 Hz, 1H), 7.39-7.15 (m, 2H), 7.12 (s, 1H), 6.80- |
| | | | | 6.52 (m, 1H), 6.05 (s, 1H), 4.79 (d, J = 14.8 Hz, 2H), |
| | | | | 4.58 (s, 2H), 4.47-4.22 (m, 1H), 4.10 (d, J = 6.8 Hz, |
| | | | | 2H), 4.04-3.96 (m, 1H), 3.88-3.61 (m, 2H), 3.60-3.41 |
| | | | | (m, 4H), 2.82-2.77 (m, 2H), 2.37 (d, J = 2.4 Hz, 1H), |
| | | | | 2.31-2.24 (m, 1H), 2.21 (d, J = 6.4 Hz, 3H), 1.38-1.23 |
| | | | | (m, 7H), 0.32-0.10 (m, 6H) |

^{a}Step 1 acylation was run for 2 hrs from 0-25° C. under standard conditions. Step 2 hydrolysis was run 0.5-2 hr at rt. Hydrolysis could also be achieved with NaOH in THF and H₂O. Purification of final compounds under standard techniques such as column chromatography and reverse-phase HPLC under a variety of conditions.
^{b} Only acylation, no hydrolysis step performed.

Compounds made via further methodology:

TABLE 19
Compounds synthesized via further methodology.

| | LCMS | |
|---|---|---|
| | (ESI+) | |
| | m/z | |
| I-# | (M + H)+ | ¹H NMR (400 MHz, DMSO-d₆) δ |
| I-1186 | 519.3 | 8.13-8.09 (m, 1H), 7.73-7.64 (m, 1H), 7.39-7.27 (m, 1H), 6.94-6.89 (m, |
| | | 1H), 6.31-6.25 (m, 1H), 4.65-4.61 (m, 2H), 4.37 (br s, 2H), 3.67-3.64 (m, |
| | | 4H), 3.59 (br d, J = 18.8 Hz, 2H), 3.27-3.23 (m, 5H), 3.12 (s, 2H), 3.06-2.99 |
| | | (m, 3H), 2.45 (br s, 2H), 2.32-2.21 (m, 2H), 2.06 (s, 3H) |
| I-1187 | 413.1 | 11.96-11.80 (m, 1H), 8.10 (d, J = 10.8 Hz, 1H), 7.73-7.62 (m, 1H), 7.38 (d, J = |
| | | 8.4 Hz, 1H), 7.05-6.93 (m, 1H), 6.84 (s, 1H), 4.64-4.56 (m, 2H), 4.52- |
| | | 4.41 (m, 1H), 3.94-3.82 (m, 1H), 3.24-3.14 (m, 3H), 3.09-2.97 (m, 6H), |
| | | 2.95-2.88 (m, 1H), 2.75-2.65 (m, 1H), 1.89-1.73 (m, 3H), 1.56-1.38 (m, 1H) |
| I-1188 | 395.3 | 11.46 (br s, 1H), 8.11 (d, J = 9.2 Hz, 1H), 7.70 (d, J = 6.0 Hz, 1H), 7.55 (d, J = |
| | | 8.4 Hz, 1H), 7.29 (d, J = 11.6 Hz, 1H), 6.99 (dd, J = 8.0, 19.2 Hz, 1H), 6.83 (s, |
| | | 1H), 4.61 (q, J = 7.2 Hz, 2H), 4.51-4.43 (m, 1H), 3.94-3.80 (m, 1H), 3.24- |
| | | 2.93 (m, 8H), 2.78-2.64 (m, 1H), 2.64-2.54 (m, 2H), 2.00-1.89 (m, 1H), |
| | | 1.81-1.68 (m, 2H), 1.55-1.36 (m, 1H) |
| I-1189 | 395.3 | 11.46 (br s, 1H), 8.11 (d, J = 9.2 Hz, 1H), 7.70 (d, J = 6.0 Hz, 1H), 7.55 (d, J = |
| | | 8.4 Hz, 1H), 7.29 (d, J = 11.6 Hz, 1H), 6.99 (dd, J = 8.0, 19.2 Hz, 1H), 6.83 (s, |
| | | 1H), 4.61 (q, J = 7.2 Hz, 2H), 4.51-4.43 (m, 1H), 3.94-3.80 (m, 1H), 3.24- |
| | | 2.93 (m, 8H), 2.78-2.64 (m, 1H), 2.64-2.54 (m, 2H), 2.00-1.89 (m, 1H), |
| | | 1.81-1.68 (m, 2H), 1.55-1.36 (m, 1H) |
| I-1190 | 465.3 | 7.47 (s, 1H), 7.44-7.38 (m, 2H), 7.34 (d, J = 6.4 Hz, 1H), 7.10-7.02 (m, 2H), |
| | | 6.12 (s, 1H), 4.48-4.39 (m, 2H), 3.83-3.80 (m, 1H), 3.80 (d, J = 3.2 Hz, 3H), |
| | | 3.70 (t, J = 5.2 Hz, 1H), 3.27 (s, 3H), 3.11 (s, 3H), 2.92-2.84 (m, 1H), 2.44- |
| | | 2.35 (m, 2H), 1.18 (t, J = 7.6 Hz, 6H) |
| I-1191 | 530.5 | 11.43-11.31 (m, 1H), 8.04 (s, 1H), 7.74-7.64 (m, 1H), 7.22-7.11 (m, 1H), |
| | | 7.09-7.03 (m, 1H), 6.90-6.78 (m, 1H), 6.54 (s, 1H), 6.30 (s, 1H), 6.24-6.15 |
| | | (m, 1H), 5.85-5.73 (m, 1H), 4.64-4.56 (m, 2H), 4.55-4.42 (m, 1H), 3.93- |
| | | 3.83 (m, 1H), 3.68-3.62 (m, 3H), 3.29 (br s, 1H), 3.21-2.98 (m, 8H), 2.74 (d, |
| | | J = 5.2 Hz, 3H), 2.61 (br t, J = 11.2 Hz, 2H), 2.02-1.92 (m, 1H), 1.81-1.70 |
| | | (m, 2H), 1.57-1.40 (m, 1H) |
| I-1192 | 476.1 | 8.33-8.17 (m, 1H), 7.63-7.36 (m, 3H), 7.25-7.12 (m, 4H), 6.12 (s, 1H), 5.41- |
| | | 5.27 (m, 1H), 4.92-4.67 (m, 1H), 4.52-4.36 (m, 2H), 4.32-4.06 (m, 1H), |
| | | 3.83-3.54 (m, 2H), 3.24 (s, 3H), 3.13-2.95 (m, 5H), 2.43-2.14 (m, 2H |
| I-1193 | 519.3 | 11.93 (s, 1H), 8.10 (d, J = 11.2 Hz, 1H), 7.69 (d, J = 10.4 Hz, 1H), 7.42-7.35 |
| | | (m, 1H), 7.32 (d, J = 7.2 Hz, 1H), 7.14 (d, J = 8.4 Hz, 1H), 7.04 (t, J = 7.2 Hz, |
| | | 1H), 6.94 (t, J = 5.2 Hz, 1H), 6.43 (s, 1H), 4.61 (q, J = 6.8 Hz, 2H), 4.49 (d, J = |
| | | 8.0 Hz, 1H), 3.90 (d, J = 12.8 Hz, 1H), 3.71 (s, 3H), 3.22-2.98 (m, 10H), 2.77- |
| | | 2.57 (m, 1H), 1.96-1.82 (m, 2H), 1.76 (d, J = 13.2 Hz, 1H), 1.56-1.39 (m, 1H) |
| I-1194 | 530.5 | 11.42-11.34 (m, 1H), 8.16-8.04 (m, 1H), 7.74-7.63 (m, 1H), 7.23-7.12 (m, |
| | | 1H), 7.08-7.04 (m, 1H), 6.91-6.76 (m, 1H), 6.50-6.46 (m, 1H), 6.31-6.28 |
| | | (m, 1H), 6.23-6.19 (m, 1H), 5.79 (br d, J = 3.6 Hz, 1H), 4.63-4.57 (m, 2H), |
| | | 4.54-4.42 (m, 1H), 3.92-3.83 (m, 1H), 3.65 (d, J = 6.8 Hz, 3H), 3.27-3.24 |
| | | (m, 1H), 3.18-2.99 (m, 8H), 2.75-2.73 (m, 3H), 2.68-2.61 (m, 2H), 1.99- |
| | | 1.92 (m, 1H), 1.82-1.72 (m, 2H), 1.55-1.38 (m, 1H) |
| I-1195 | 519.2 | 12.07-11.79 (m, 1H), 8.10 (d, J = 11.6 Hz, 1H), 7.69 (d, J = 10.4 Hz, 1H), |
| | | 7.42-7.35 (m, 1H), 7.32 (br d, J = 7.6 Hz, 1H), 7.14 (d, J = 8.0 Hz, 1H), 7.04 |
| | | (t, J = 7.6 Hz, 1H), 6.94 (t, J = 5.6 Hz, 1H), 5.76 (s, 1H), 4.61 (q, J = 6.4 Hz, |
| | | 2H), 4.48 (br t, J = 8.8 Hz, 1H), 3.93-3.87 (m, 1H), 3.71 (s, 3H), 3.32 (s, 6H), |
| | | 3.08-3.01 (m, 5H), 1.97-1.82 (m, 2H), 1.77 (br d, J = 11.2 Hz, 1H), 1.61- |
| | | 1.37 (m, 1H) |
| I-1196 | 578.4 | |
| I-1197 | 578.4 | 8.35-8.19 (m, 2H), 7.89-7.76 (m, 1H), 7.73-7.65 (m, 1H), 7.57-7.50 (m, |
| | | 1H), 7.46-7.42 (m, 1H), 7.42-7.38 (m, 1H), 7.29-7.21 (m, 5H), 7.20-7.12 |
| | | (m, 6H), 7.11-6.99 (m, 2H), 6.35 (br s, 1H), 5.55-5.44 (m, 1H), 5.41-5.34 |
| | | (m, 1H), 4.93-4.81 (m, 1H), 4.81-4.71 (m, 1H), 4.62-4.50 (m, 2H), 4.47 (br |
| | | d, J = 12 Hz, 2H), 4.43-4.35 (m, 1H), 4.34-4.19 (m, 1H), 3.70 (s, 2H), 3.70- |
| | | 3.66 (m, 4H), 3.22 (br s, 6H), 3.11-2.94 (m, 8H), 2.35-2.23 (m, 7H) |
| I-1198 | 679.3 | |
| I-1199 | 696.3 | |
| I-1200 | 657.3 | |
| I-1201 | 654.3 | |
| I-1202 | 648.4 | 1.17-1.33 (m, 2H), 2.24-2.31 (m, 2H), 2.44 (br s, 3H), 2.83 (s, 1H), 2.90 (s, |
| | | 1H), 2.93-3.03 (m, 2H), 3.45-3.53 (m, 3H), 3.70 (d, J = 2.13 Hz, 3H), 3.83 |
| | | (br s, 3H), 4.22 (br d, J = 1.76 Hz, 1H), 4.42 (br d, J = 1.76 Hz, 1H), 4.66-4.80 |
| | | (m, 2H), 6.01-6.11 (m, 1H), 6.45-6.53 (m, 1H), 6.89-7.04 (m, 3H), 7.18- |
| | | 7.25 (m, 1H), 7.26 (s, 2H), 7.28-7.37 (m, 2H), 7.59 (dd, J = 2.89, 0.63 Hz, |
| | | 1H), 7.71 (dd, J = 4.02, 0.63 Hz, 1H), 7.96 (s, 1H), 9.75-9.91 (m, 1H) |

TABLE 20
Compounds synthesized via further methodology.

| | LCMS | |
|---|---|---|
| | (ESI+) | |
| | m/z | |
| I-# | (M + H)+ | ¹H NMR (400 MHz, DMSO-d₆) δ |
| I-1236 | 466.1 | 11.88 (s, 1H), 7.89 (br s, 1H), 7.81 (d, J = 8.4 Hz, 2H), 7.38 (d, J = 8.4 Hz, 1H), |
| | | 7.31 (d, J = 8.4 Hz, 2H), 7.30-7.26 (m, 1H), 7.18-7.14 (m, 1H), 7.04-6.98 |
| | | (m, 1H), 6.84 (d, J = 2.8 Hz, 1H), 4.29 (br d, J = 5.6 Hz, 2H), 4.07 (br d, J = |
| | | 12.4 Hz, 2H), 3.29-3.16 (m, 3H), 3.12-2.98 (m, 4H), 2.94-2.86 (m, 1H), 2.82- |
| | | 2.73 (m, 1H), 1.89 (br d, J = 10.4 Hz, 1H), 1.83-1.69 (m, 2H), 1.57-1.46 (m, 1H) |
| I-1235 | 466.2 | 11.89 (s, 1H), 7.90 (br s, 1H), 7.82 (d, J = 8.4 Hz, 2H), 7.39 (d, J = 8.4 Hz, 1H), |
| | | 7.36-7.25 (m, 3H), 7.17 (br t, J = 5.6 Hz, 1H), 7.02 (dd, J = 6.0, 8.1 Hz, 1H), |
| | | 6.85 (d, J = 2.8 Hz, 1H), 4.29 (br d, J = 5.6 Hz, 2H), 4.08 (br d, J = 12.5 Hz, |
| | | 2H), 3.29-2.97 (m, 7H), 2.96-2.85 (m, 1H), 2.78 (br t, J = 12.0 Hz, 1H), 1.94- |
| | | 1.85 (m, 1H), 1.83-1.68 (m, 2H), 1.60-1.45 (m, 1H) |

### Example I-379: Synthesis of Methyl 5-(3-(5-(2-(dimethylcarbamoyl)benzofuran-5-yl)-1,2,3,6-tetrahydropyridine-1-carbonyl)-2-formyl-1,2,3,4-tetrahydroisoquinolin-5-yl)pent-4-ynoate (I-802)

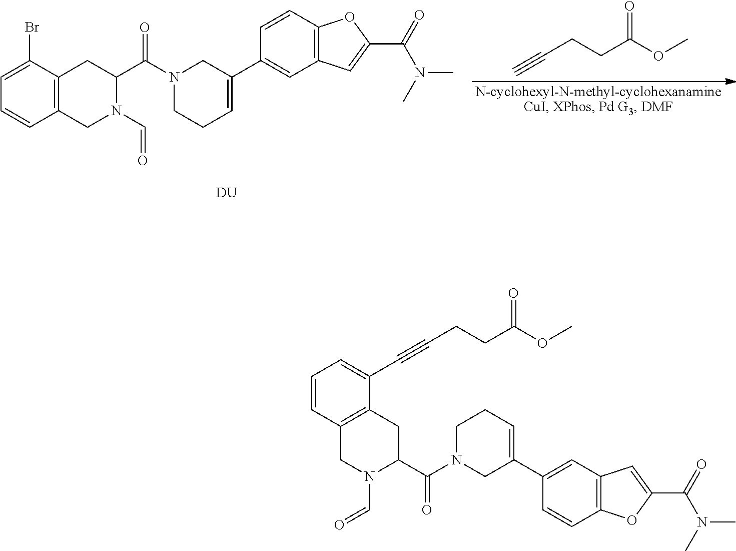

A mixture of 5-(1-(5-bromo-2-formyl-1,2,3,4-tetrahydroisoquinoline-3-carbonyl)-1,2,5,6-tetrahydropyridin-3-yl)-N,N-dimethylbenzofuran-2-carboxamide (100 mg, 186 umol, Intermediate DU), methyl pent-4-ynoate (104 mg, 932 umol, CAS #21565-82-2), copper iodide (35 mg, 186 umol), N-cyclohexyl-N-methyl-cyclohexanamine (182 mg, 932 umol, 198 uL) and methanesulfonato(2-dicyclohexylphosphino-2,4,6-tri-1-propyl-1,1-biphenyl)(2-amino-1,1-biphenyl-2-yl)palladium(II) (16 mg, 18.6 umol) in dimethylformamide (1.0 mL) was degassed and purged with nitrogen three times. Then the mixture was stirred at 90° C. for 12 h under nitrogen atmosphere. On completion, the mixture was diluted with water (10 mL) and extracted with ethyl acetate (10 mL×2). The combined organic layers were washed with brine (10 mL) and dried with sodium sulfate solid and filtered. Then the filtrate was concentrated under reduced pressure to dryness. The residue was purified by prep-TLC (silica gel, petroleum ether:ethyl acetate=2:1) to afford an impurity product (40 mg, 42% purity). The impurity product was purified by prep-HPLC (column: Phenomenex luna C18 150*25 mm*10 um;mobile phase: [water(FA)-ACN];B %: 32%-62%,10 min). The aqueous solution was lyophilized in vacuo to give the title compound (4.48 mg, 4% yield) as a yellow solid. LC-MS (ESI⁺) m/z 568.4 (M+H)⁺; ¹H NMR (400 MHz, DMSO-d₆) δ=8.37-8.25 (m, 1H), 7.91-7.71 (m, 1H), 7.71-7.46 (m, 2H), 7.42-7.31 (m, 1H), 7.30-7.02 (m, 3H), 6.33 (d, J=1.6 Hz, 1H), 5.64-5.43 (m, 1H), 4.93-4.69 (m, 1H), 4.65-4.13 (m, 3H), 3.73-3.49 (m, 4H), 3.27 (s, 6H), 3.18-2.92 (m, 4H), 2.76-2.62 (m, 2H), 2.57 (d, J=6.4 Hz, 1H), 2.43-2.33 (m, 1H), 2.29-2.12 (m, 1H).

### Examples I-380, I-381, and I-382: Syntheses of 5-(1-(2-formyl-1,2,3,4-tetrahydroisoquinoline-3-carbonyl)-1,2,5,6-tetrahydropyridin-3-yl)-7-(2-methoxyphenyl)-N,N-dimethylbenzofuran-2-carboxamide (I-803), (R)-5-(1-(2-formyl-1,2,3,4-tetrahydroisoquinoline-3-carbonyl)-1,2,5,6-tetrahydropyridin-3-yl)-7-(2-methoxyphenyl)-N,N-dimethylbenzofuran-2-carboxamide (I-804) and (S)-5-(1-(2-formyl-1,2,3,4-tetrahydroisoquinoline-3-carbonyl)-1,2,5,6-tetrahydropyridin-3-yl)-7-(2-methoxyphenyl)-N,N-dimethylbenzofuran-2-carboxamide (I-805)

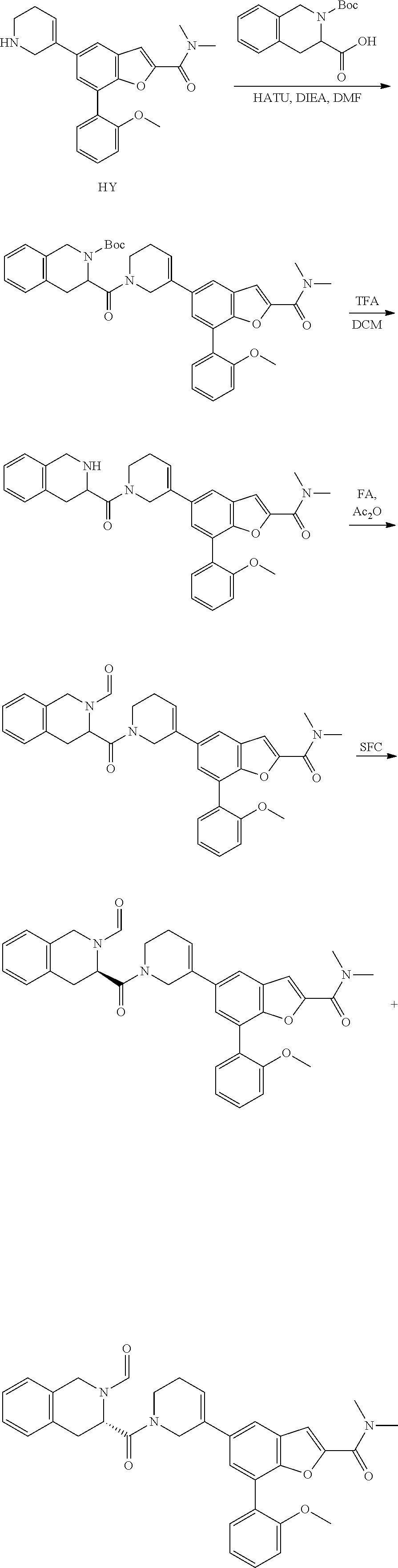

### Step 1—tert-butyl 3-(5-(2-(dimethylcarbamoyl)-7-(2-methoxyphenyl)benzofuran-5-yl)-1,2,3,6-tetrahydropyridine-1-carbonyl)-3,4-dihydroisoquinoline-2(1H)-carboxylate

To a solution of 2-(tert-butoxycarbonyl)-1,2,3,4-tetrahydroisoquinoline-3-carboxylic acid (221 mg, 796 μmol, CAS #151838-62-9) in DMF (5 mL) was added HATU (393 mg, 1.04 mmol), DIEA (514 mg, 3.98 mmol, 694 μL), and 7-(2-methoxyphenyl)-N,N-dimethyl-5-(1,2,5,6-tetrahydropyridin-3-yl)benzofuran-2-carboxamide (300 mg, 796 μmol, Intermediate HY). Then the mixture was stirred at 25° C. for 0.5 hrs. On completion, the reaction mixture was diluted with water (10 mL) and extracted with dichloromethane (2×10 mL). The combined organic layers were washed with brine (20 mL) and dried over anhydrous sodium sulfate, filtered and the filtrate was concentrated in vacuo to give the title compound (500 mg, 99% yield) as yellow solid. LC-MS (ESI⁺) m/z 636.3 (M+H)⁺.

### Step 2—Tert-butyl 3-(5-(2-(dimethylcarbamoyl)-7-(2-methoxyphenyl)benzofuran-5-yl)-1,2,3,6-tetrahydropyridine-1-carbonyl)-3,4-dihydroisoquinoline-2(1H)-carboxylate

To a solution of 7-(2-methoxyphenyl)-N,N-dimethyl-5-(1-(1,2,3,4-tetrahydroisoquinoline-3-carbonyl)-1,2,5,6-tetrahydropyridin-3-yl)benzofuran-2-carboxamide (500 mg, 786 μmol) in DCM (5 mL) was added TFA (89.6 mg, 786 μmol, 58.4 μL). The mixture was stirred at 25° C. for 2 hr. On completion, the reaction mixture was concentrated in vacuo to give the title compound (520 mg, TFA) as yellow oil. LC-MS (ESI⁺) m/z 536.3 (M+H)⁺.

### Step 3—5-(1-(2-formyl-1,2,3,4-tetrahydroisoquinoline-3-carbonyl)-1,2,5,6-tetrahydropyridin-3-yl)-7-(2-methoxyphenyl)-N,N-dimethylbenzofuran-2-carboxamide

To a solution of 7-(2-methoxyphenyl)-N,N-dimethyl-5-(1-(1,2,3,4-tetrahydroisoquinoline-3-carbonyl)-1,2,5,6-tetrahydropyridin-3-yl)benzofuran-2-carboxamide (520 mg, 800 μmol, TFA) in HCOOH (10 mL) and Ac₂O (10 mL). The mixture was then stirred at 25° C. for 4 hrs. On completion, NaHCO₃ was added to the reaction mixture until pH=5 and the mixture was diluted with water (20 mL) and extracted with ethyl acetate (2×20 mL). The combined organic layers were dried over anhydrous sodium sulfate, filtered and the filtrate was concentrated in vacuo to give the crude residue. The crude residue was purified by reversed-phase HPLC (0.1% FA condition) to give the title compound (350 mg, 76% yield) as a white solid. LC-MS (ESI⁺) m/z 564.3 (M+H)⁺; ¹H NMR (400 MHz, DMSO-d₆) δ=8.34-8.23 (m, 1H), 7.87-7.66 (m, 1H), 7.56-7.39 (m, 4H), 7.24-7.14 (m, 5H), 7.11-7.04 (m, 1H), 6.39-6.31 (m, 1H), 5.55-5.32 (m, 1H), 4.90-4.69 (m, 1H), 4.59-4.49 (m, 1H), 4.49-4.24 (m, 2H), 3.73 (br d, J=7.2 Hz, 4H), 3.65-3.53 (m, 1H), 3.20 (br s, 3H), 3.10-2.93 (m, 5H), 2.41-2.28 (m, 2H).

### Step 4—(R)-5-(1-(2-formyl-1,2,3,4-tetrahydroisoquinoline-3-carbonyl)-1,2,5,6-tetrahydropyridin-3-yl)-7-(2-methoxyphenyl)-N,N-dimethylbenzofuran-2-carboxamide and (S)-5-(1-(2-formyl-1,2,3,4-tetrahydroisoquinoline-3-carbonyl)-1,2,5,6-tetrahydropyridin-3-yl)-7-(2-methoxyphenyl)-N,N-dimethylbenzofuran-2-carboxamide

5-(1-(2-Formyl-1,2,3,4-tetrahydroisoquinoline-3-carbonyl)-1,2,5,6-tetrahydropyridin-3-yl)-7-(2-methoxyphenyl)-N,N-dimethylbenzofuran-2-carboxamide was purified by SFC (column: DAICEL CHIRALPAK AD-H(250 mm×30 mm,5 um); mobile phase: [CO₂-i-PrOH/ACN];B %:50%, isocratic elution mode) to give the first eluting peak (R)-5-(1-(2-formyl-1,2,3,4-tetrahydroisoquinoline-3-carbonyl)-1,2,5,6-tetrahydropyridin-3-yl)-7-(2-methoxyphenyl)-N,N-dimethylbenzofuran-2-carboxamide (90 mg, 30% yield) as a white solid (LC-MS (ESI⁺) m/z 564.0 (M+H)⁺; ¹H NMR (400 MHz, DMSO-d₆) δ=8.33-8.22 (m, 1H), 7.87-7.66 (m, 1H), 7.57-7.36 (m, 4H), 7.25-7.14 (m, 5H), 7.12-7.03 (m, 1H), 6.38-6.30 (m, 1H), 5.55-5.34 (m, 1H), 4.94-4.69 (m, 1H), 4.54 (br d, J=13.6 Hz, 1H), 4.47-4.17 (m, 2H), 3.78-3.68 (m, 4H), 3.44 (br s, 1H), 3.24-3.15 (m, 3H), 3.13 (br s, 5H), 2.26 (br s, 2H)) and the second eluting peak as (S)-5-(1-(2-formyl-1,2,3,4-tetrahydroisoquinoline-3-carbonyl)-1,2,5,6-tetrahydropyridin-3-yl)-7-(2-methoxyphenyl)-N,N-dimethylbenzofuran-2-carboxamide (80 mg, 25.60% yield, 96% purity) as a white solid (LC-MS (ESI⁺) m/z 564.0 (M+H)⁺; ¹H NMR (400 MHz, DMSO-d₆) δ=8.33-8.22 (m, 1H), 7.87-7.66 (m, 1H), 7.57-7.36 (m, 4H), 7.25-7.14 (m, 5H), 7.12-7.03 (m, 1H), 6.38-6.30 (m, 1H), 5.55-5.34 (m, 1H), 4.94-4.69 (m, 1H), 4.54 (br d, J=13.6 Hz, 1H), 4.47-4.17 (m, 2H), 3.78-3.68 (m, 4H), 3.44 (br s, 1H), 3.24-3.15 (m, 3H), 3.13 (br s, 5H), 2.26 (br s, 2H)). The absolute stereochemistry of the enantiomers was assigned arbitrarily.

### Example I-383: Synthesis of 1-(5-(4-bromo-7-fluoro-2-(4-(5-fluoro-3-methoxypyridin-2-yl)piperazine-1-carbonyl)-1H-indol-6-yl)-3,6-dihydropyridin-1(2H)-yl)-3-(1H-1,2,3-triazol-1-yl)propan-1-one (I-806)

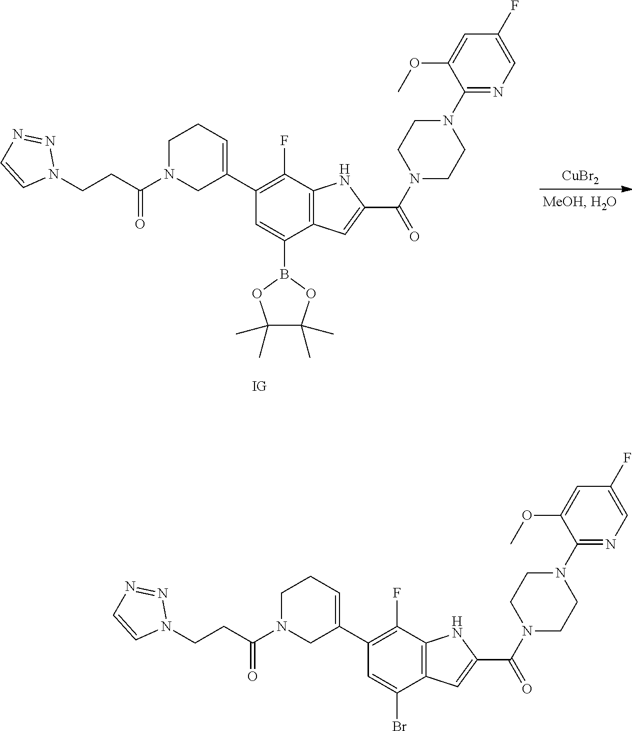 [SMILES: COc1cc(F)cnc1N1CCN(C(=O)c2cc3c(B4OC(C)(C)C(C)(C)O4)cc(C4=CCCN(C(=O)CCn5ccnn5)C4)c(F)c3[nH]2)CC1 ; "CuBr2" ; COc1cc(F)cnc1N1CCN(C(=O)c2cc3c(Br)cc(C4=CCCN(C(=O)CCn5ccnn5)C4)c(F)c3[nH]2)CC1]

A mixture of 1-[5-[7-fluoro-2-[4-(5-fluoro-3-methoxy-2-pyridyl)piperazine-1-carbonyl]-4-(4,4,5,5-tetramethyl-1,3,2-dioxaborolan-2-yl)-1H-indol-6-yl]-3,6-dihydro-2H-pyridin-1-yl]-3-(triazol-1-yl)propan-1-one (160 mg, 227 μmol, Intermediate IG), CuBr₂ (153 mg, 683 μmol) and H₂O (1.00 g, 55.5 mmol, 1 mL) in MeOH (2.00 mL) was degassed and purged with N₂ three times. Then the mixture was stirred at 55° C. for 18 hrs under N₂ atmosphere. On completion, the reaction mixture was filtered and concentrated under reduced pressure. The crude product was purified by reversed-phase HPLC(column: CD20-Waters Xbridge BEH C18 250×25×10 um; mobile phase: [water(NH₄HCO₃)-ACN]; gradient:35%-65% B over 15 min) to give the title compound (37.2 mg, 24% yield) as a white solid. LC-MS (ESI⁺) m/z 657.1 (M+H)⁺; ¹H NMR (400 MHz, DMSO-d₆) δ=12.63-12.50 (m, 1H), 8.14-8.08 (m, 1H), 7.80 (d, J=2.4 Hz, 1H), 7.72-7.66 (m, 1H), 7.38 (dd, J=2.4, 10.4 Hz, 1H), 7.30-7.19 (m, 1H), 6.71 (s, 1H), 6.15-6.10 (m, 1H), 4.62 (q, J=6.8 Hz, 2H), 4.33-4.26 (m, 2H), 3.85 (s, 4H), 3.82 (s, 3H), 3.63 (t, J=5.6 Hz, 1H), 3.58 (t, J=5.6 Hz, 1H), 3.29 (s, 4H), 3.09 (d, J=5.6 Hz, 2H), 2.33 (s, 2H).

### Example I-384: Synthesis of 1-(6-(1-(3-(1H-1,2,3-triazol-1-yl)propanoyl)-1,2,5,6-tetrahydropyridin-3-yl)-7-fluoro-2-(4-(3-methoxypyridin-2-yl)piperazine-1-carbonyl)-1H-indol-4-yl)pyridin-2(1H)-one (I-807)

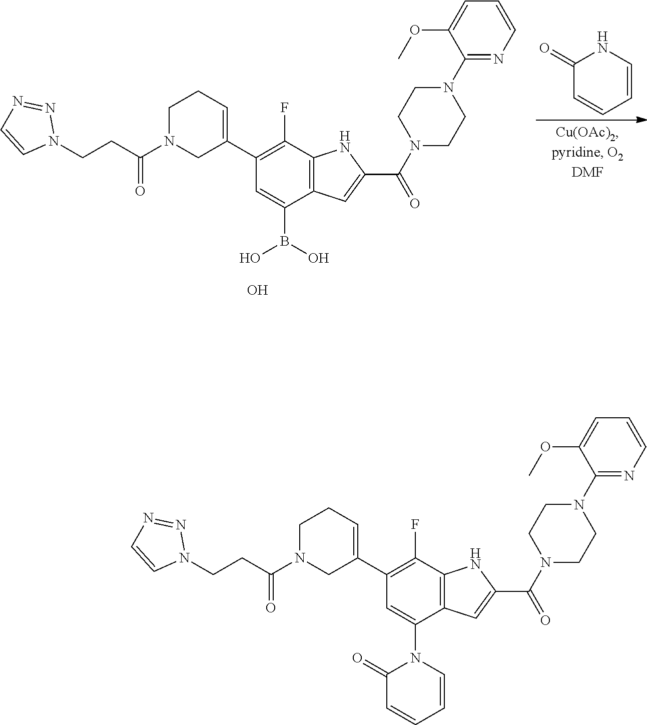

A mixture of [7-fluoro-2-[4-(3-methoxy-2-pyridyl)piperazine-1-carbonyl]-6-[1-[3-(triazol-1-yl)propanoyl]-3,6-dihydro-2H-pyridin-5-yl]-1H-indol-4-yl]boronic acid (200 mg, 331 μmol, Intermediate OH), 1H-pyridin-2-one (34.7 mg, 365 μmol, CAS #142-08-5), Cu(OAc)₂H₂O (46.4 mg, 232 μmol) and pyridine (54.8 mg, 693 μmol) in DMF (3 mL) was degassed and purged with O₂ three times. Then the mixture was stirred at 50° C. for 1 hr under O₂ atmosphere (15 psi). On completion, the mixture was diluted with H₂O (8 mL) and extracted with EA (3 mL×4). The combined organic layers were washed with aqueous NaCl (4 mL×2), dried over Na₂SO₄, filtered and concentrated under reduced pressure to give a residue. The crude product was purified by reversed-phase HPLC (column: CD09-Phenomenex Gemini C18 150*30*5 um; mobile phase: [water(NH₄HCO₃)-ACN];gradient:22%-42% B over 10 min) to afford the title compound (8.14 mg, 4% yield) as a white solid. LC-MS (ESI⁺) m/z 652.4 (M+H)⁺; ¹H NMR (400 MHz, DMSO-d₆) δ=12.52-12.39 (m, 1H), 8.13-8.09 (m, 1H), 7.81-7.77 (m, 1H), 7.71-7.65 (m, 2H), 7.58-7.53 (m, 1H), 7.27 (dd, J=1.2, 8.0 Hz, 1H), 7.12-6.99 (m, 1H), 6.92 (dd, J=4.8, 8.0 Hz, 1H), 6.52 (d, J=9.6 Hz, 1H), 6.43 (s, 1H), 6.34 (t, J=6.4 Hz, 1H), 6.21-6.14 (m, 1H), 4.62 (q, J=7.2 Hz, 2H), 4.38-4.30 (m, 2H), 3.80 (s, 3H), 3.78 (s, 4H), 3.66-3.58 (m, 2H), 3.33-3.33 (m, 4H), 3.12-3.07 (m, 2H), 2.35 (d, J=2.0 Hz, 1H), 2.31-2.24 (m, 1H).

### Examples I-385 & I-386: Syntheses of (S)-1-(5-(7-fluoro-2-(4-(5-fluoro-3-methoxypyridin-2-yl)piperazine-1-carbonyl)-4-(1-methylpyrrolidin-3-yl)-1H-indol-6-yl)-3,6-dihydropyridin-1(2H)-yl)-3-(1H-1,2,3-triazol-1-yl)propan-1-one (I-811) and (R)-1-(3-(7-fluoro-2-(4-(5-fluoro-3-methoxypyridin-2-yl)piperazine-1-carbonyl)-4-(1-methylpyrrolidin-3-yl)-1H-indol-6-yl)-5,6-dihydropyridin-1(2H)-yl)-3-(1H-1,2,3-triazol-1-yl)propan-1-one (I-812)

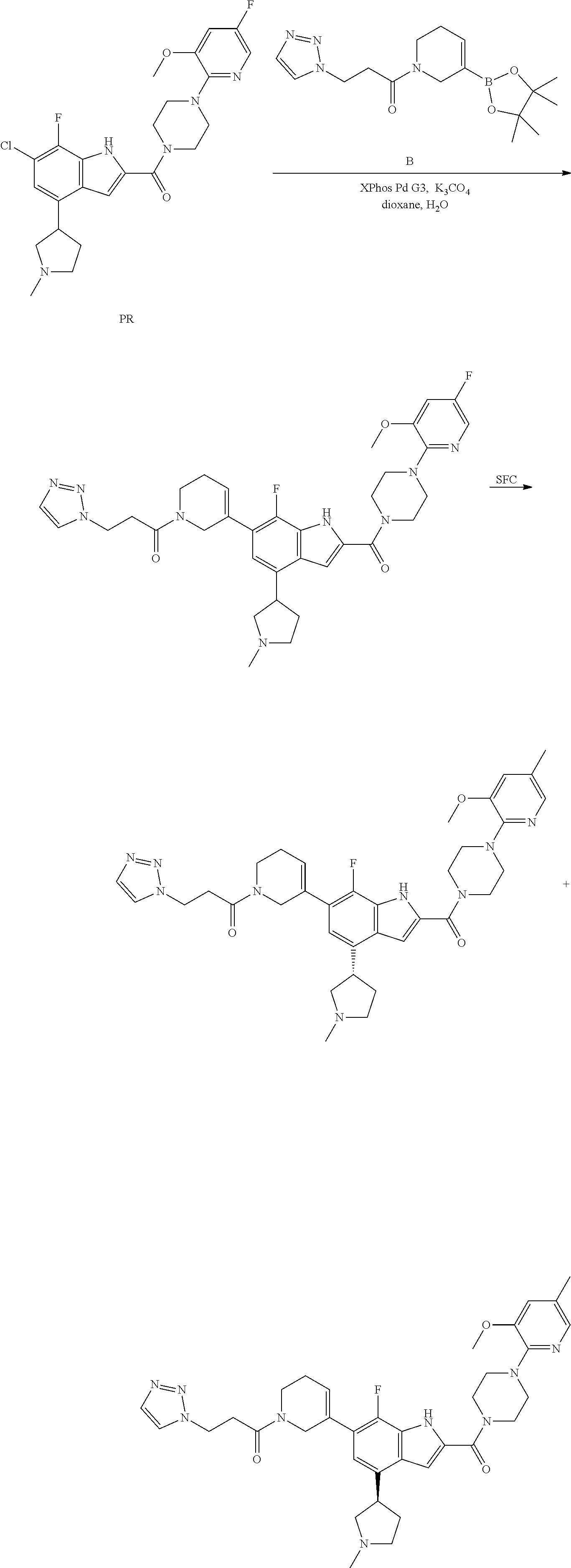

### Step 1—1-(3-(7-Fluoro-2-(4-(5-fluoro-3-methoxypyridin-2-yl)piperazine-1-carbonyl)-4-(1-methylpyrrolidin-3-yl)-1H-indol-6-yl)-5,6-dihydropyridin-1(2H)-yl)-3-(1H-1,2,3-triazol-1-yl)propan-1-one

A mixture of [6-chloro-7-fluoro-4-(1-methylpyrrolidin-3-yl)-1H-indol-2-yl]-[4-(5-fluoro-3-methoxy-2-pyridyl)piperazin-1-yl]methanone (100 mg, 204 μmol, Intermediate PR), 1-[5-(4,4,5,5-tetramethyl-1,3,2-dioxaborolan-2-yl)-3,6-dihydro-2H-pyridin-1-yl]-3-(triazol-1-yl)propan-1-one (67.80 mg, 204.10 μmol, Intermediate B), XPhos Pd G3 (17.28 mg, 20.41 μmol), and K₂CO₃ (84.63 mg, 612.3 mol) in dioxane (1.00 mL) and H₂O (0.30 mL) was degassed and purged with N₂ three times. Then the mixture was stirred at 80° C. for 1 hr under N₂ atmosphere. Upon completion, the mixture was filtered to obtain crude reaction solution. The crude product was purified by reversed-phase HPLC(0.1% FA condition) to give the title compound (130 mg, 90% yield) as a white solid. LC-MS (ESI⁺) m/z 660.3 (M+H)⁺.

### Step 2—(S)-1-(5-(7-fluoro-2-(4-(5-fluoro-3-methoxypyridin-2-yl)piperazine-1-carbonyl)-4-(1-methylpyrrolidin-3-yl)-1H-indol-6-yl)-3,6-dihydropyridin-1(2H)-yl)-3-(1H-1,2,3-triazol-1-yl)propan-1-one and (R)-1-(3-(7-fluoro-2-(4-(5-fluoro-3-methoxypyridin-2-yl)piperazine-1-carbonyl)-4-(1-methylpyrrolidin-3-yl)-1H-indol-6-yl)-5,6-dihydropyridin-1(2H)-yl)-3-(1H-1,2,3-triazol-1-yl)propan-1-one

1-(3-(7-Fluoro-2-(4-(5-fluoro-3-methoxypyridin-2-yl)piperazine-1-carbonyl)-4-(1-methylpyrrolidin-3-yl)-1H-indol-6-yl)-5,6-dihydropyridin-1(2H)-yl)-3-(1H-1,2,3-triazol-1-yl)propan-1-one (100 mg, 152 μmol) was separated by SFC (column: DAICEL CHIRALPAK IK(250 mm*30 mm,10 um);mobile phase: [CO₂-ACN/MeOH(0.1% NH₃H2O)];B %:60%, isocratic elution mode, RT=Peak 1: 3.674 min, Peak 2: 6.013). Then each peak was purified by reversed-phase HPLC (0.1% FA condition) to give the first eluting peak (S)-1-(5-(7-fluoro-2-(4-(5-fluoro-3-methoxypyridin-2-yl)piperazine-1-carbonyl)-4-(1-methylpyrrolidin-3-yl)-1H-indol-6-yl)-3,6-dihydropyridin-1(2H)-yl)-3-(1H-1,2,3-triazol-1-yl)propan-1-one (19.34 mg, 18% yield) as a white solid (LC-MS (ESI⁺) m/z 660.3 (M+H)⁺; ¹H NMR (400 MHz, DMSO-d6) δ (ppm)=12.06 (s, 1H), 8.15-8.07 (m, 1H), 7.80 (d, J=2.0 Hz, 1H), 7.74-7.63 (m, 1H), 7.41-7.35 (m, 1H), 7.03 (s, 1H), 6.92-6.82 (m, 1H), 6.05 (s, 1H), 4.66-4.60 (m, 2H), 4.34-4.26 (m, 2H), 3.86 (s, 3H), 3.83 (s, 4H), 3.70 (d, J=7.2 Hz, 2H), 3.32-3.30 (m, 2H), 3.30-3.27 (m, 2H), 3.15-3.03 (m, 3H), 2.93 (t, J=7.6 Hz, 1H), 2.82-2.77 (m, 1H), 2.74-2.66 (m, 2H), 2.38 (s, 3H), 2.34 (s, 2H), 2.29-2.25 (m, 1H), 1.99-1.88 (m, 1H)) and the second eluting peak (R)-1-(3-(7-fluoro-2-(4-(5-fluoro-3-methoxypyridin-2-yl)piperazine-1-carbonyl)-4-(1-methylpyrrolidin-3-yl)-1H-indol-6-yl)-5,6-dihydropyridin-1(2H)-yl)-3-(1H-1,2,3-triazol-1-yl)propan-1-one (31.09 mg, 28% yield) as a white solid (LC-MS (ESI⁺) m/z 660.3 (M+H)⁺; ¹H NMR (400 MHz, DMSO-d6) δ (ppm)=12.06 (s, 1H), 8.15-8.08 (m, 1H), 7.80 (s, 1H), 7.72-7.65 (m, 1H), 7.40-7.35 (m, 1H), 7.03 (s, 1H), 6.90-6.83 (m, 1H), 6.05 (s, 1H), 4.62 (d, J=6.4 Hz, 2H), 4.33-4.27 (m, 2H), 3.86 (s, 3H), 3.83 (s, 4H), 3.70 (d, J=6.0 Hz, 2H), 3.33-3.30 (m, 2H), 3.30-3.28 (m, 2H), 3.13-3.05 (m, 3H), 2.93 (s, 1H), 2.80 (d, J=4.8 Hz, 1H), 2.74-2.67 (m, 2H), 2.38 (s, 3H), 2.33 (s, 2H), 2.27 (s, 1H), 1.97-1.88 (m, 1H)). The absolute stereochemistry of the enantiomers was assigned arbitrarily.

### Example I-387: Synthesis of 3-(3-(4-(4-ethyl-6-methylpyridin-3-yl)-7-fluoro-2-(4-(5-fluoro-3-methoxypyridin-2-yl)piperazine-1-carbonyl)-1H-indol-6-yl)-5,6-dihydropyridin-1(2H)-yl)-2-methyl-3-oxopropanoic acid (I-808)

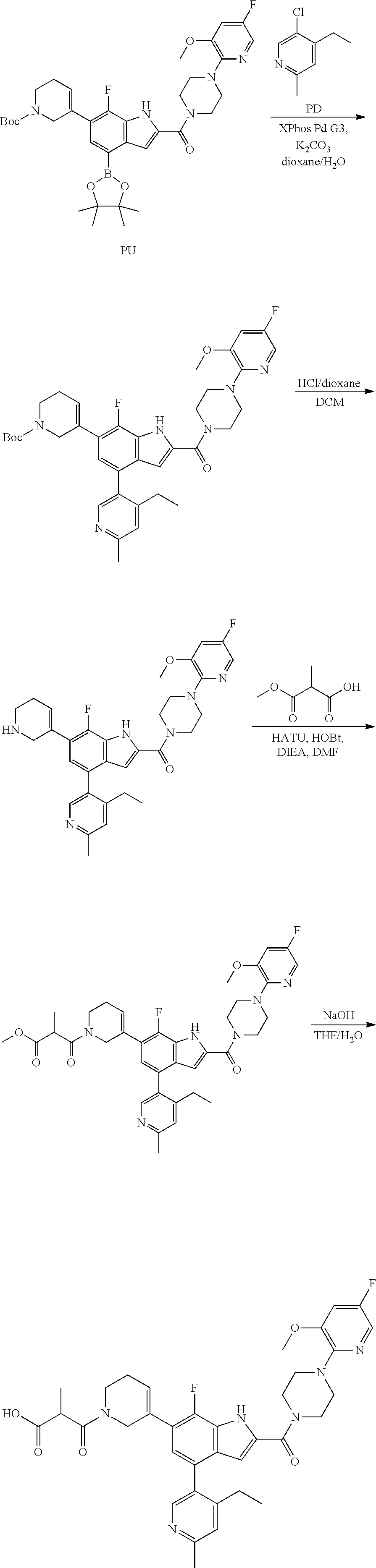

### Step 1—tert-butyl 3-(4-(4-ethyl-6-methylpyridin-3-yl)-7-fluoro-2-(4-(5-fluoro-3-methoxypyridin-2-yl)piperazine-1-carbonyl)-1H-indol-6-yl)-5,6-dihydropyridine-1(2H)-carboxylate

To a solution of tert-butyl 3-(7-fluoro-2-(4-(5-fluoro-3-methoxypyridin-2-yl)piperazine-1-carbonyl)-4-(4,4,5,5-tetramethyl-1,3,2-dioxaborolan-2-yl)-1H-indol-6-yl)-5,6-dihydropyridine-1(2H)-carboxylate (500.00 mg, 735.77 μmol, Intermediate PU) and 5-chloro-4-ethyl-2-methyl-pyridine (114.50 mg, 735.77 μmol, Intermediate PD) in dioxane (5 mL) and H₂O (1 mL) were added K₂CO₃ (305.06 mg, 2.21 mmol) and XPhos Pd G₃ (62.28 mg, 73.58 μmol). The mixture was then stirred at 100° C. for 1 h. The residue was diluted with H₂O (2 mL) and extracted with EtOAc (5 mL×2). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 0˜100% EtOAc/Petroleum ether gradient @60 mL/min) and prep-TLC (SiO₂, EtOAc/Petroleum ether=1:1) to give the title compound (300.00 mg, 42% yield) as a white solid. LC-MS (ESI⁺) m/z 673.3 (M+H)⁺.

### Step 2—(4-(4-Ethyl-6-methylpyridin-3-yl)-7-fluoro-6-(1,2,5,6-tetrahydropyridin-3-yl)-1H-indol-2-yl)(4-(5-fluoro-3-methoxypyridin-2-yl)piperazin-1-yl)methanone

To a solution of tert-butyl 3-(4-(4-ethyl-6-methylpyridin-3-yl)-7-fluoro-2-(4-(5-fluoro-3-methoxypyridin-2-yl)piperazine-1-carbonyl)-1H-indol-6-yl)-5,6-dihydropyridine-1(2H)-carboxylate (300.00 mg, 445.92 μmol) in DCM (2 mL) was added HCl/dioxane (2 M, 4 mL). The mixture was stirred at 20° C. for 1 h. Upon completion, the mixture was concentrated under reduced pressure to give a residue. The residue was purified by prep-HPLC (column: CD01-Phenomenex luna C18 150*25*10 um; mobile phase: [water (FA)-ACN]; gradient: 5%-35% B over 10 min) to give the title compound (120.00 mg, 46% yield) as a white solid. LC-MS (ESI⁺) m/z 573.3 (M+H)⁺; ¹H NMR (400 MHz, DMSO-d₆) δ=12.32-12.11 (m, 1H), 8.30 (s, 1H), 8.26 (s, 1H), 7.78 (d, J=2.4 Hz, 1H), 7.36 (dd, J=2.4, 10.0 Hz, 1H), 7.28 (s, 1H), 6.82 (d, J=6.0 Hz, 1H), 6.35 (d, J=3.2 Hz, 1H), 6.11 (s, 1H), 3.83 (s, 3H), 3.76 (s, 4H), 3.63 (s, 2H), 3.23 (s, 6H), 2.92 (t, J=5.6 Hz, 2H), 2.49-2.42 (m, 3H), 2.22 (s, 2H), 0.99 (t, J=7.6 Hz, 3H).

### Step 3—Methyl 3-(3-(4-(4-ethyl-6-methylpyridin-3-yl)-7-fluoro-2-(4-(5-fluoro-3-methoxypyridin-2-yl)piperazine-1-carbonyl)-1H-indol-6-yl)-5,6-dihydropyridin-1(2H)-yl)-2-methyl-3-oxopropanoate

To a solution of (4-(4-ethyl-6-methylpyridin-3-yl)-7-fluoro-6-(1,2,5,6-tetrahydropyridin-3-yl)-1H-indol-2-yl)(4-(5-fluoro-3-methoxypyridin-2-yl)piperazin-1-yl)methanone (110.00 mg, 192.09 μmol) and 3-methoxy-2-methyl-3-oxo-propanoic acid (30.45 mg, 230.5 μmol, CAS #3097-74-3), DIEA (148.95 mg, 1.15 mmol, 200.75 μL), and HOBt (51.91 mg, 384.18 μmol) in DMF (3 mL) was added HATU (87.65 mg, 230.5 μmol). The mixture was then stirred at 20° C. for 0.5 h. Upon completion, the mixture was diluted with H₂O (2 mL) and extracted with EtOAc (5 mL×2). The combined organic layers were dried over Na₂SO₄, filtered and concentrated under reduced pressure to give a residue. The residue was purified by prep-TLC (SiO₂, Petroleum ether: EtOAc=0:1) to give the title compound (100.00 mg, 72% yield) as a white solid. LC-MS (ESI⁺) m/z 687.7 (M+H)⁺.

### Step 4—3-(3-(4-(4-Ethyl-6-methylpyridin-3-yl)-7-fluoro-2-(4-(5-fluoro-3-methoxypyridin-2-yl)piperazine-1-carbonyl)-1H-indol-6-yl)-5,6-dihydropyridin-1(2H)-yl)-2-methyl-3-oxopropanoic acid

To a solution of methyl 3-(3-(4-(4-ethyl-6-methylpyridin-3-yl)-7-fluoro-2-(4-(5-fluoro-3-methoxypyridin-2-yl)piperazine-1-carbonyl)-1H-indol-6-yl)-5,6-dihydropyridin-1(2H)-yl)-2-methyl-3-oxopropanoate (100.00 mg, 145.61 μmol) in THF (1 mL) was added NaOH (3 M, 242.69 μL). The mixture was stirred at 20° C. for 1 h. Upon completion, the mixture was adjusted to pH=5 with 2M HCl. The residue was extracted with EtOAc (2 mL×3). The combined organic layers were dried over Na₂SO₄, filtered and concentrated under reduced pressure to give a residue. The residue was purified by prep-HPLC (column: CD02-Waters Xbridge BEH C18 150*25*10 um; mobile phase: [water (NH₄HCO₃)-ACN]; gradient: 16%-46% B over 10 min) to give the title compound (31.17 mg, 32% yield) as a white solid. LC-MS (ESI⁺) m/z 673.3 (M+H)⁺; ¹H NMR (400 MHz, DMSO-d₆) δ=12.52-11.96 (m, 1H), 8.28 (s, 1H), 7.78 (d, J=2.4 Hz, 1H), 7.35 (dd, J=2.4, 10.4 Hz, 1H), 7.28 (s, 1H), 7.00-6.75 (m, 1H), 6.35 (d, J=3.2 Hz, 1H), 6.16 (s, 1H), 4.40 (d, J=18.0 Hz, 2H), 3.83 (s, 4H), 3.76 (s, 4H), 3.71-3.61 (m, 2H), 3.24 (s, 4H), 2.51 (s, 3H), 2.45 (s, 2H), 2.36 (d, J=2.4 Hz, 1H), 2.31-2.22 (m, 1H), 1.25-1.13 (m, 3H), 1.00 (t, J=7.6 Hz, 3H).

### Examples I-388 & I-389: Syntheses of (1S)-3-[4-(4-ethyl-3-pyridyl)-7-fluoro-2-[4-(5-fluoro-3-methoxy-2-pyridyl)piperazine-1-carbonyl]-1H-indol-6-yl]cyclohex-3-ene-1-carboxylic acid (I-809) and (1R)-3-[4-(4-ethyl-3-pyridyl)-7-fluoro-2-[4-(5-fluoro-3-methoxy-2-pyridyl)piperazine-1-carbonyl]-1H-indol-6-yl]cyclohex-3-ene-1-carboxylic acid (I-810)

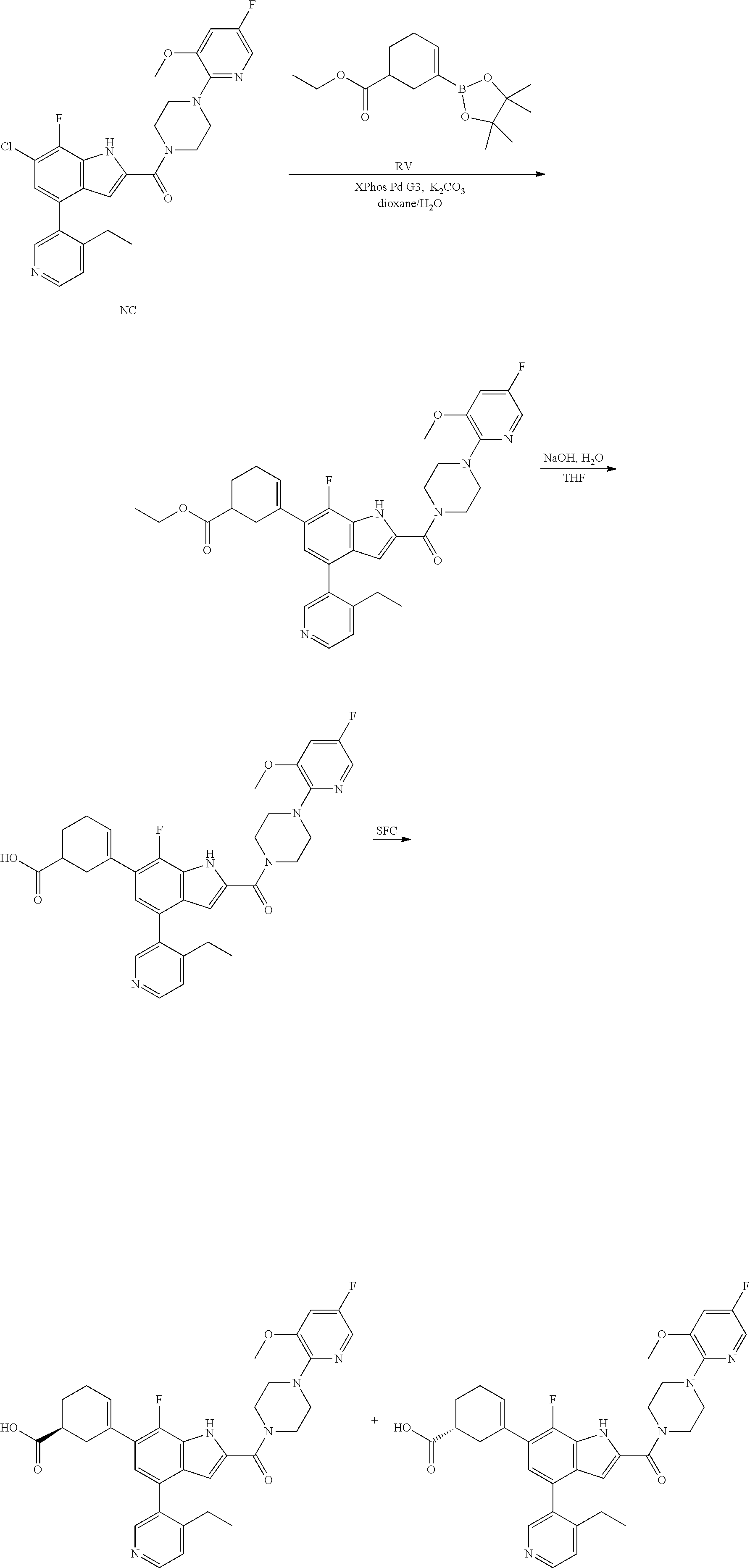

### Step 1—Ethyl 3-[4-(4-ethyl-3-pyridyl)-7-fluoro-2-[4-(5-fluoro-3-methoxy-2-pyridyl)piperazine-1-carbonyl]-1H-indol-6-yl]cyclohex-3-ene-1-carboxylate

A mixture of ethyl 3-(4,4,5,5-tetramethyl-1,3,2-dioxaborolan-2-yl)cyclohex-3-ene-1-carboxylate (287 mg, 1.03 mmol, Intermediate RV), [6-chloro-4-(4-ethyl-3-pyridyl)-7-fluoro-1H-indol-2-yl]-[4-(5-fluoro-3-methoxy-2-pyridyl)piperazin-1-yl]methanone (350 mg, 684 μmol, Intermediate NC), XPhos Pd G3 (58 mg, 68 μmol), and K₂CO₃ (189 mg, 1.37 mmol) in dioxane (8 mL) and H₂O (1 mL) was degassed and purged with N₂ three times. Then the mixture was stirred at 80° C. for 1 h under N₂ atmosphere. Upon completion, the mixture was filtered and the filtrate was concentrated under reduced pressure to give the residue. The residue was purified by flash silica gel chromatography (ISCO®; 12 g SepaFlash® Silica Flash Column, Eluent of 0˜55% EtOAc/Petroleum ether gradient @70 mL/min) to give the title compound (180 mg, 30% yield) as a yellow oil. LC-MS (ESI⁺) m/z 630.3 (M+H)⁺; ¹H NMR (400 MHz, DMSO-d₆) δ=12.40-12.10 (m, 1H), 7.77 (d, J=2.4 Hz, 1H), 7.51-7.41 (m, 2H), 7.35 (dd, J=2.4, 10.4 Hz, 1H), 6.86 (d, J=6.4 Hz, 1H), 6.38-6.29 (s, 1H), 6.00 (s, 1H), 4.09 (q, J=6.8 Hz, 2H), 3.83 (s, 3H), 3.80-3.69 (m, 4H), 3.27-3.19 (m, 4H), 2.80-2.69 (m, 1H), 2.68-2.58 (m, 2H), 2.45-2.23 (m, 5H), 2.00 (br s, 1H), 1.97 (br s, 1H), 1.75-1.62 (m, 1H), 1.04-0.98 (m, 6H).

### Step 2—3-[4-(4-Ethyl-3-pyridyl)-7-fluoro-2-[4-(5-fluoro-3-methoxy-2-pyridyl)piperazine-1-carbonyl]-1H-indol-6-yl]cyclohex-3-ene-1-carboxylic acid

To a solution of ethyl 3-[4-(4-ethyl-3-pyridyl)-7-fluoro-2-[4-(5-fluoro-3-methoxy-2-pyridyl)piperazine-1-carbonyl]-1H-indol-6-yl]cyclohex-3-ene-1-carboxylate (180 mg, 286 μmol) in THF (3 mL) was added NaOH (2 M, 3 mL). The mixture was then stirred at 20° C. for 48 h. Upon completion, the pH was adjusted to 5 by addition of 2M HCl, and then extracted with EtOAc (5 mL×5). The combined organic layers were concentrated under reduced pressure to give a residue. The residue was purified by prep-TLC (SiO₂, DCM:MeOH=10:1) to give the title compound (105 mg, 58% yield) as a white solid. LC-MS (ESI⁺) m/z 602.2 (M+H)⁺.

### Step 3—(1S)-3-[4-(4-ethyl-3-pyridyl)-7-fluoro-2-[4-(5-fluoro-3-methoxy-2-pyridyl)piperazine-1-carbonyl]-1H-indol-6-yl]cyclohex-3-ene-1-carboxylic acid

3-[4-(4-ethyl-3-pyridyl)-7-fluoro-2-[4-(5-fluoro-3-methoxy-2-pyridyl)piperazine-1-carbonyl]-1H-indol-6-yl]cyclohex-3-ene-1-carboxylic acid (0.1 g, 166.21 μmol) was separated by SFC (column: DAICEL CHIRALPAK AD(250 mm*30 mm,10 um);mobile phase: [CO₂-ACN/i-PrOH(0.1% NH₃H2O)];B %:45%, isocratic elution mode) to give the first eluting isomer as (1S)-3-[4-(4-ethyl-3-pyridyl)-7-fluoro-2-[4-(5-fluoro-3-methoxy-2-pyridyl)piperazine-1-carbonyl]-1H-indol-6-yl]cyclohex-3-ene-1-carboxylic acid (14.46 mg, 14% yield) as a white solid (LC-MS (ESI⁺) m/z 602.3 (M+H)⁺; ¹H NMR (400 MHz, DMSO-d₆) δ=12.25 (s, 1H), 8.52 (d, J=4.8 Hz, 1H), 8.40 (s, 1H), 7.78 (d, J=2.4 Hz, 1H), 7.42 (d, J=5.2 Hz, 1H), 7.35 (dd, J=2.4, 10.4 Hz, 1H), 6.86 (d, J=6.0 Hz, 1H), 6.38-6.29 (m, 1H), 6.07-5.96 (m, 1H), 3.83 (s, 3H), 3.80-3.71 (m, 4H), 3.28-3.11 (m, 5H), 2.68-2.53 (m, 4H), 2.35-2.20 (m, 2H), 2.05-1.92 (m, 1H), 1.76-1.51 (m, 1H), 1.00 (t, J=7.6 Hz, 3H)) and the second eluting isomer (1R)-3-[4-(4-ethyl-3-pyridyl)-7-fluoro-2-[4-(5-fluoro-3-methoxy-2-pyridyl)piperazine-1-carbonyl]-1H-indol-6-yl]cyclohex-3-ene-1-carboxylic acid (19.43 mg, 19% yield) as a white solid (LC-MS (ESI⁺) m/z 602.3 (M+H)⁺; 12.43-12.08 (m, 1H), 8.52 (d, J=4.8 Hz, 1H), 8.40 (s, 1H), 7.77 (d, J=2.4 Hz, 1H), 7.41 (d, J=5.2 Hz, 1H), 7.35 (dd, J=2.4, 10.4 Hz, 1H), 6.85 (d, J=6.0 Hz, 1H), 6.33 (d, J=2.8 Hz, 1H), 6.04-5.94 (m, 1H), 3.83 (s, 3H), 3.79-3.72 (m, 4H), 3.25-3.16 (m, 5H), 2.68-2.52 (m, 4H), 2.35-2.22 (m, 2H), 2.04-1.92 (m, 1H), 1.70-1.56 (m, 1H), 1.00 (t, J=7.6 Hz, 3H)). The absolute configuration of the enantiomers was assigned arbitrarily.

### Examples I-390 & I-391: Syntheses of 1-[5-[7-fluoro-2-[4-(5-fluoro-3-methoxy-2-pyridyl)piperazine-1-carbonyl]-4-[(2S)-1-methylpyrrolidin-2-yl]-1H-indol-6-yl]-3,6-dihydro-2H-pyridin-1-yl]-3-(triazol-1-yl)propan-1-one (I-813) and 1-[5-[7-fluoro-2-[4-(5-fluoro-3-methoxy-2-pyridyl)piperazine-1-carbonyl]-4-[(2R)-1-methylpyrrolidin-2-yl]-1H-indol-6-yl]-3,6-dihydro-2H-pyridin-1-yl]-3-(triazol-1-yl)propan-1-one (I-814)

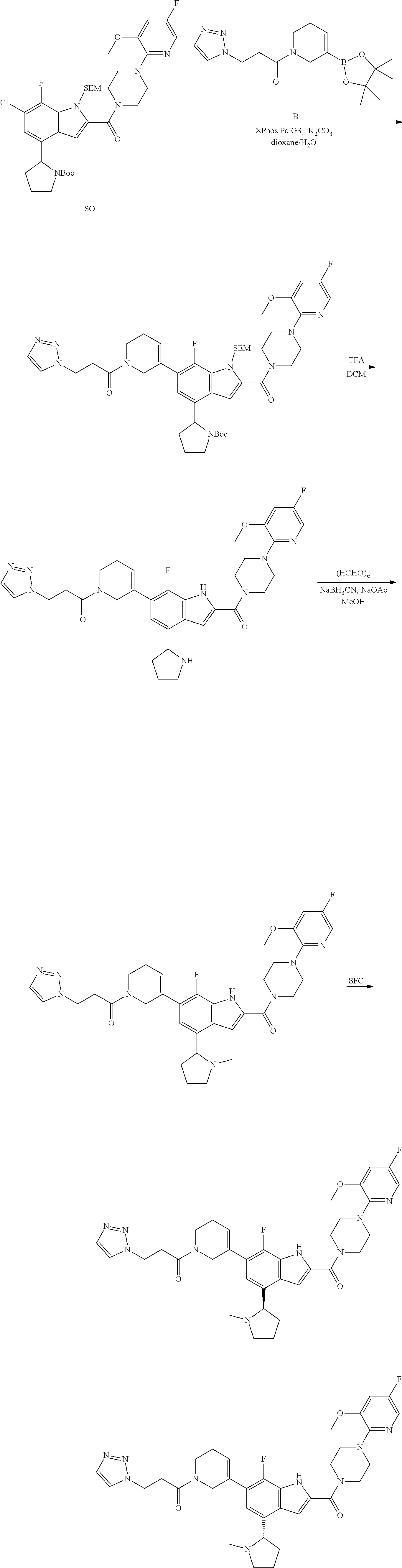

### Step 1—Tert-butyl 2-[7-fluoro-2-[4-(5-fluoro-3-methoxy-2-pyridyl)piperazine-1-carbonyl]-6-[1-[3-(triazol-1-yl)propanoyl]-3,6-dihydro-2H-pyridin-5-yl]-1-(2-trimethylsilylethoxymethyl)indol-4-yl]pyrrolidine-1-carboxylate

To a solution of tert-butyl 2-[6-chloro-7-fluoro-2-[4-(5-fluoro-3-methoxy-2-pyridyl)piperazine-1-carbonyl]-1-(2-trimethylsilylethoxymethyl)indol-4-yl]pyrrolidine-1-carboxylate (650 mg, 920 μmol, Intermediate SO) and 1-[5-(4,4,5,5-tetramethyl-1,3,2-dioxaborolan-2-yl)-3,6-dihydro-2H-pyridin-1-yl]-3-(triazol-1-yl)propan-1-one (306 mg, 920 μmol, Intermediate B) in dioxane (10 mL) and H₂O (1 mL) was added XPhos Pd G3 (78 mg, 92 μmol) and K₂CO₃ (382 mg, 2.76 mmol). The mixture was then stirred at 80° C. for 4 h. Upon completion, the aqueous phase was separated and the organic phase was filtered and the filtrate was concentrated under reduced pressure to give the residue. The residue was purified by flash silica gel chromatography (ISCO®; 20 g SepaFlash® Silica Flash Column, Eluent of 0˜95% EtOAc/Petroleum ether gradient @70 mL/min) to give the title compound (420 mg, 50% yield) as a yellow oil. LC-MS (ESI⁺) m/z 876.5 (M+H)+

### Step 2—1-[5-[7-Fluoro-2-[4-(5-fluoro-3-methoxy-2-pyridyl)piperazine-1-carbonyl]-4-pyrrolidin-2-yl-1H-indol-6-yl]-3,6-dihydro-2H-pyridin-1-yl]-3-(triazol-1-yl)propan-1-one

To a solution of tert-butyl 2-[7-fluoro-2-[4-(5-fluoro-3-methoxy-2-pyridyl)piperazine-1-carbonyl]-6-[1-[3-(triazol-1-yl)propanoyl]-3,6-dihydro-2H-pyridin-5-yl]-1-(2-trimethylsilylethoxymethyl)indol-4-yl]pyrrolidine-1-carboxylate (320 mg, 365 μmol) in DCM (9 mL) was added TFA (3.84 g, 33.7 mmol, 2.5 mL). Then the mixture was stirred at 20° C. for 1 h. Upon completion, the mixture was concentrated under reduced pressure to give the title compound (300 mg, TFA) as a green oil. LC-MS (ESI⁺) m/z 646.4 (M+H)⁺.

### Step 3—1-[5-[7-Fluoro-2-[4-(5-fluoro-3-methoxy-2-pyridyl)piperazine-1-carbonyl]-4-(1-methylpyrrolidin-2-yl)-1H-indol-6-yl]-3,6-dihydro-2H-pyridin-1-yl]-3-(triazol-1-yl)propan-1-one

To a solution of 1-[5-[7-fluoro-2-[4-(5-fluoro-3-methoxy-2-pyridyl)piperazine-1-carbonyl]-4-pyrrolidin-2-yl-1H-indol-6-yl]-3,6-dihydro-2H-pyridin-1-yl]-3-(triazol-1-yl)propan-1-one (110 mg, 170 μmol) in MeOH (5 mL) were added (HCHO)˜(80 mg, 170 μmol, CAS #30525-89-4), NaOAc (27.95 mg, 340.7 μmol) and NaBH₃CN (16 mg, 260 μmol). The mixture was then stirred at 20° C. for 4 h. Upon completion, the mixture was concentrated under reduced pressure to give the residue. The residue was purified by prep-TLC (SiO₂, DCM:MeOH=10:1) to give the title compound (105 mg, 84% yield) as a white solid. LC-MS (ESI⁺) m/z 660.4 (M+H)⁺.

### Step 4—1-[5-[7-fluoro-2-[4-(5-fluoro-3-methoxy-2-pyridyl)piperazine-1-carbonyl]-4-[(2S)-1-methylpyrrolidin-2-yl]-1H-indol-6-yl]-3,6-dihydro-2H-pyridin-1-yl]-3-(triazol-1-yl)propan-1-one and 1-[5-[7-fluoro-2-[4-(5-fluoro-3-methoxy-2-pyridyl)piperazine-1-carbonyl]-4-[(2R)-1-methylpyrrolidin-2-yl]-1H-indol-6-yl]-3,6-dihydro-2H-pyridin-1-yl]-3-(triazol-1-yl)propan-1-one

1-[5-[7-fluoro-2-[4-(5-fluoro-3-methoxy-2-pyridyl)piperazine-1-carbonyl]-4-(1-methylpyrrolidin-2-yl)-1H-indol-6-yl]-3,6-dihydro-2H-pyridin-1-yl]-3-(triazol-1-yl)propan-1-one (105 mg, 159 μmol) was separated by SFC (column: DAICEL CHIRALCEL OD(250 mm*30 mm,10 um);mobile phase: [CO₂-MeOH(0.1% NH₃H₂O)];B %:40%, isocratic elution mode) to give the first eluting isomer as 1-[5-[7-fluoro-2-[4-(5-fluoro-3-methoxy-2-pyridyl)piperazine-1-carbonyl]-4-[(2S)-1-methylpyrrolidin-2-yl]-1H-indol-6-yl]-3,6-dihydro-2H-pyridin-1-yl]-3-(triazol-1-yl)propan-1-one (21.06 mg, 20% yield) as an off-white solid (LC-MS (ESI⁺) m/z 660.4 (M+H)⁺; ¹H NMR (400 MHz, DMSO-d₆) δ=12.31-11.86 (m, 1H), 8.27-8.05 (m, 1H), 7.81 (d, J=2.0 Hz, 1H), 7.75-7.64 (m, 1H), 7.38 (dd, J=2.4, 10.4 Hz, 1H), 7.10-6.86 (m, 2H), 6.18-5.99 (m, 1H), 4.62 (q, J=6.4 Hz, 2H), 4.40-4.23 (m, 2H), 3.86 (s, 3H), 3.85-3.78 (m, 4H), 3.65 (t, J=5.2 Hz, 1H), 3.60 (t, J=5.2 Hz, 1H), 3.50-3.38 (m, 1H), 3.31-3.26 (m, 4H), 3.24-3.15 (m, 1H), 3.14-3.03 (m, 2H), 2.39-2.24 (m, 4H), 2.22-2.03 (m, 3H), 1.97-1.57 (m, 3H) and the second eluting isomer as 1-[5-[7-fluoro-2-[4-(5-fluoro-3-methoxy-2-pyridyl)piperazine-1-carbonyl]-4-[(2R)-1-methylpyrrolidin-2-yl]-1H-indol-6-yl]-3,6-dihydro-2H-pyridin-1-yl]-3-(triazol-1-yl)propan-1-one (22.63 mg, 21% yield) as an off-white solid (LC-MS (ESI⁺) m/z 660.4 (M+H)⁺; ¹H NMR (400 MHz, DMSO-d₆) δ=12.05 (s, 1H), 8.16-8.07 (m, 1H), 7.80 (d, J=2.4 Hz, 1H), 7.73-7.66 (m, 1H), 7.38 (dd, J=2.4, 10.4 Hz, 1H), 7.02-6.93 (m, 2H), 6.11-6.00 (m, 1H), 4.62 (q, J=6.8 Hz, 2H), 4.39-4.25 (m, 2H), 3.86 (s, 3H), 3.85-3.76 (m, 4H), 3.65 (t, J=5.6 Hz, 1H), 3.59 (t, J=5.6 Hz, 1H), 3.49-3.39 (m, 1H), 3.30-3.25 (mz, 4H), 3.24-3.15 (m, 1H), 3.13-3.02 (m, 2H), 2.38-2.22 (m, 4H), 2.18-2.05 (m, 3H), 1.94-1.76 (m, 2H), 1.73-1.60 (m, 1H)). The absolute stereochemistry was assigned arbitrarily.

### Example I-392: Synthesis of 2-(3-(1-(cyclopropylmethyl)-7-(2-ethyl-6-methylpyridin-3-yl)-5-(4-(5-fluoro-3-methoxypyridin-2-yl)piperazine-1-carbonyl)-1H-indol-2-yl)-5,6-dihydropyridin-1(2H)-yl)-2-oxoacetic acid (I-1228)

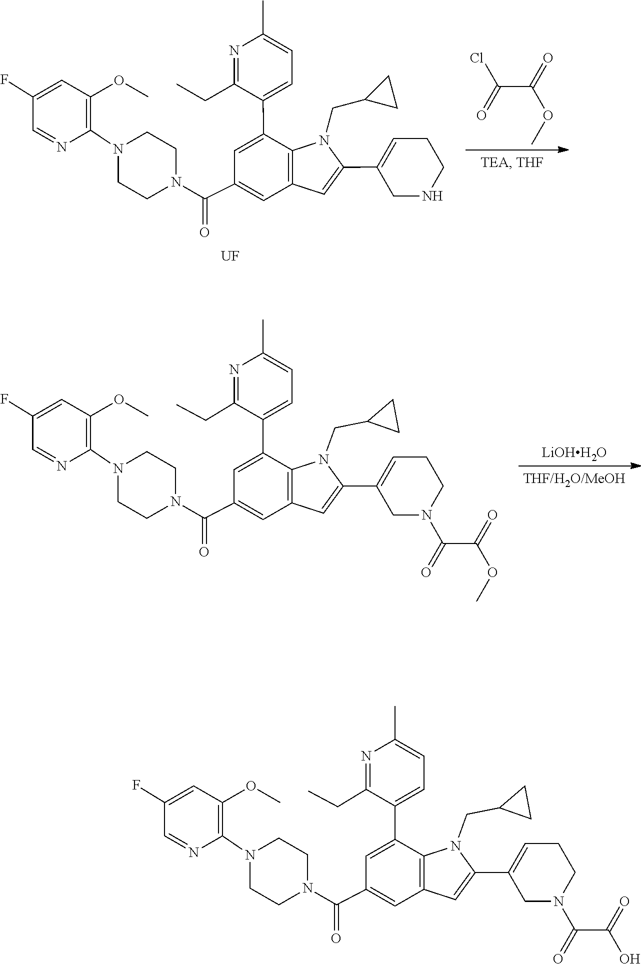

### Step 1—Methyl 2-(3-(1-(cyclopropylmethyl)-7-(2-ethyl-6-methylpyridin-3-yl)-5-(4-(5-fluoro-3-methoxypyridin-2-yl)piperazine-1-carbonyl)-1H-indol-2-yl)-5,6-dihydropyridin-1(2H)-yl)-2-oxoacetate

To a solution of [1-(cyclopropylmethyl)-7-(2-ethyl-6-methyl-3-pyridyl)-2-(1,2,3,6-tetrahydropyridin-5-yl)indol-5-yl]-[4-(5-fluoro-3-methoxy-2-pyridyl)piperazin-1-yl]methanone (180 mg, 278 μmol, HCl, Intermediate UF) and TEA (84.7 mg, 836 μmol) in DCM (2 mL) was added methyl 2-chloro-2-oxo-acetate (51.2 mg, 418 μmol, CAS #5781-53-3) and stirred at 0° C. for 1 hr. On completion, the reaction mixture was partitioned between H₂O (10 mL) and EA (20 mL). The organic phase was separated, washed with EA (20 mL×3). The combined organic layers were washed with brine (20 mL), dried over anhydrous sodium sulfate, filtered and the filtrate was concentrated in vacuo to give the title compound (130 mg, 67% yield) as a yellow solid. LC-MS (ESI⁺) m/z 695.3 (M+H)⁺.

### Step 2—2-(3-(1-(cyclopropylmethyl)-7-(2-ethyl-6-methylpyridin-3-yl)-5-(4-(5-fluoro-3-methoxypyridin-2-yl)piperazine-1-carbonyl)-1H-indol-2-yl)-5,6-dihydropyridin-1(2H)-yl)-2-oxoacetic acid

To a solution of methyl 2-[5-[1-(cyclopropylmethyl)-7-(2-ethyl-6-methyl-3-pyridyl)-5-[4-(5-fluoro-3-methoxy-2-pyridyl)piperazine-1-carbonyl]indol-2-yl]-3,6-dihydro-2H-pyridin-1-yl]-2-oxo-acetate (110 mg, 158 μmol) in THF (2 mL), H₂O (0.4 mL) and MeOH (0.4 mL) was added LiOH·H₂O (13.3 mg, 316 μmol). The mixture was then stirred at 40° C. for 0.5 hrs. On completion, to the reaction mixture was added FA until the pH=6 and the mixture was concentrated in vacuo to give a residue. The residue was purified by prep-HPLC (column: CD01-Phenomenex luna C18 150*25*10 um;mobile phase: [water(FA)-ACN];gradient:16%-46% B over 10 min) to give the title compound (48.2 mg, 41% yield, FA) as a white solid. LC-MS (ESI⁺) m/z 681.5 (M+H)⁺; ¹H NMR (400 MHz, DMSO-d6) δ=7.78 (d, J=2.0 Hz, 1H), 7.72-7.67 (m, 2H), 7.36 (dd, J=2.4, 10.4 Hz, 1H), 7.22 (d, J=8.0 Hz, 1H), 6.92 (s, 1H), 6.74-6.61 (m, 1H), 6.06-5.98 (m, 1H), 4.38-4.22 (m, 1H), 4.15-4.04 (m, 1H), 3.83 (s, 3H), 3.69-3.62 (m, 4H), 3.54-3.51 (m, 2H), 3.24 (s, 6H), 2.55 (s, 3H), 2.47-2.38 (m, 2H), 2.37-2.30 (m, 2H), 1.05-1.00 (m, 3H), 0.54-0.45 (m, 1H), 0.15-0.05 (m, 2H), −0.18-−0.27 (m, 2H).

### Examples I-393 and I-394: Syntheses of 1-[5-[4-(4-ethyl-1-methyl-pyrrolidin-3-yl)-7-fluoro-2-[4-(5-fluoro-3-methoxy-2-pyridyl)piperazine-1-carbonyl]-1H-indol-6-yl]-3,6-dihydro-2H-pyridin-1-yl]-3-(triazol-1-yl)propan-1-one (I-1208) and 1-[5-[4-(4-ethyl-1-methyl-pyrrolidin-3-yl)-7-fluoro-2-[4-(5-fluoro-3-methoxy-2-pyridyl)piperazine-1-carbonyl]-1H-indol-6-yl]-3,6-dihydro-2H-pyridin-1-yl]-3-(triazol-1-yl)propan-1-one (I-1209)

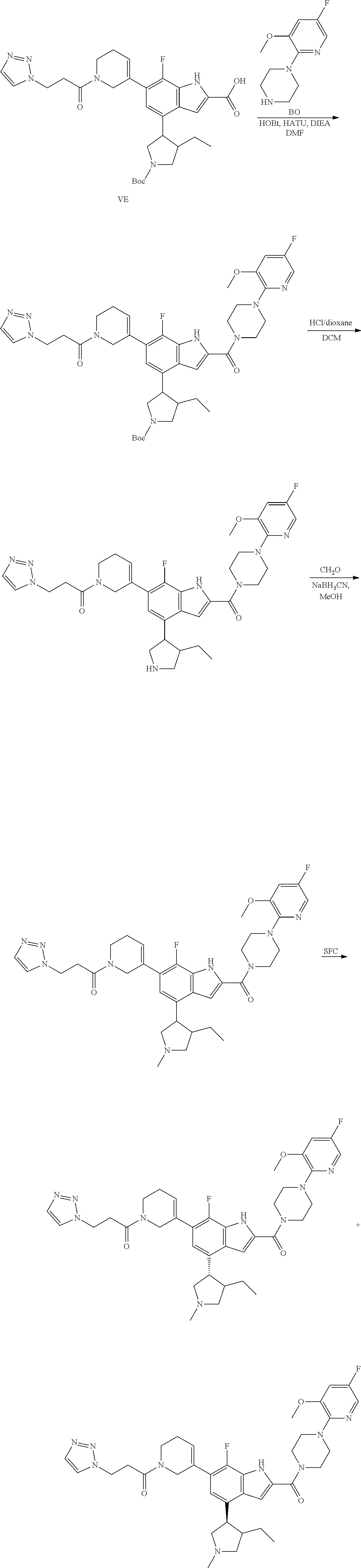

### Step 1—Tert-butyl 3-ethyl-4-[7-fluoro-2-[4-(5-fluoro-3-methoxy-2-pyridyl)piperazine-1-carbonyl]-6-[1-[3-(triazol-1-yl)propanoyl]-3,6-dihydro-2H-pyridin-5-yl]-1H-indol-4-yl]pyrrolidine-1-carboxylate

To a solution of 4-(1-tert-butoxycarbonyl-4-ethyl-pyrrolidin-3-yl)-7-fluoro-6-[1-[3-(triazol-1-yl)propanoyl]-3,6-dihydro-2H-pyridin-5-yl]-1H-indole-2-carboxylic acid (830 mg, 1.43 mmol, Intermediate VE), 1-(5-fluoro-3-methoxy-2-pyridyl)piperazine (354 mg, 1.43 mmol, HCl, Intermediate BO), HOBt (386 mg, 2.86 mmol) and DIEA (1.11 g, 8.58 mmol, 1.49 mL) in DMF (10 mL) was added and HATU (652 mg, 1.72 mmol). Then the mixture was stirred at 20° C. for 0.5 hr. On completion, the reaction mixture was diluted with H₂O (5 mL) and extracted with EtOAc (10 mL×2). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 0˜100% EtOAc/PE gradient @100 mL/min) to give the title compound (900 mg, 73% yield) as a white solid. LC-MS (ESI⁺) m/z 774.4 (M+1)⁺; ¹H NMR (400 MHz, DMSO-d₆) δ=12.19-12.03 (m, 1H), 8.16-8.05 (m, 1H), 7.80 (d, J=2.0 Hz, 1H), 7.73-7.63 (m, 1H), 7.45-7.32 (m, 1H), 7.02-6.88 (m, 2H), 6.09 (br d, J=2.8 Hz, 1H), 4.65-4.60 (m, 2H), 4.35-4.26 (m, 2H), 3.86 (s, 3H), 3.83-3.78 (m, 2H), 3.77-3.56 (m, 6H), 3.30-3.28 (m, 2H), 3.12-3.07 (m, 2H), 3.02-2.94 (m, 1H), 2.39-2.20 (m, 4H), 1.40 (br d, J=14.8 Hz, 12H), 1.29-1.23 (m, 3H), 0.79-0.73 (m, 3H).

### Step 2—1-[5-[4-(4-ethylpyrrolidin-3-yl)-7-fluoro-2-[4-(5-fluoro-3-methoxy-2-pyridyl)piperazine-1-carbonyl]-1H-indol-6-yl]-3,6-dihydro-2H-pyridin-1-yl]-3-(triazol-1-yl)propan-1-one

To a solution of tert-butyl 3-ethyl-4-[7-fluoro-2-[4-(5-fluoro-3-methoxy-2-pyridyl)piperazine-1-carbonyl]-6-[1-[3-(triazol-1-yl)propanoyl]-3,6-dihydro-2H-pyridin-5-yl]-1H-indol-4-yl]pyrrolidine-1-carboxylate (400 mg, 517 μmol) in DCM (1 mL) was added HCl/dioxane (2 M, 3.00 mL). The mixture was then stirred at 20° C. for 1 hr. On completion, the reaction mixture was concentrated under reduced pressure to give a residue. The residue was purified by prep-HPLC (FA condition) to give the title compound (350 mg, 91% yield) as a white solid. LC-MS (ESI⁺) m/z 674.3 (M+1)⁺.

### Step 3—1-[5-[4-(4-ethyl-1-methyl-pyrrolidin-3-yl)-7-fluoro-2-[4-(5-fluoro-3-methoxy-2-pyridyl)piperazine-1-carbonyl]-1H-indol-6-yl]-3,6-dihydro-2H-pyridin-1-yl]-3-(triazol-1-yl)propan-1-one

To a solution of 1-[5-[4-(4-ethylpyrrolidin-3-yl)-7-fluoro-2-[4-(5-fluoro-3-methoxy-2-pyridyl)piperazine-1-carbonyl]-1H-indol-6-yl]-3,6-dihydro-2H-pyridin-1-yl]-3-(triazol-1-yl)propan-1-one (150 mg, 223 μmol) and (HCHO)ₙ (222.63 μmol) in MeOH (2 mL) was added NaBH₃CN (28 mg, 450 mol). The mixture was then stirred at 20° C. for 1 hr. On completion, the reaction mixture was filtered and the filtrate was concentrated under reduced pressure to give a residue. The residue was purified by prep-HPLC (FA condition) to give the title compound (70 mg, 41% yield) as a white solid. LC-MS (ESI⁺) m/z 688.4 (M+1)⁺.

### Step 4—1-[5-[4-(4-ethyl-1-methyl-pyrrolidin-3-yl)-7-fluoro-2-[4-(5-fluoro-3-methoxy-2-pyridyl)piperazine-1-carbonyl]-1H-indol-6-yl]-3,6-dihydro-2H-pyridin-1-yl]-3-(triazol-1-yl)propan-1-one and 1-[5-[4-(4-ethyl-1-methyl-pyrrolidin-3-yl)-7-fluoro-2-[4-(5-fluoro-3-methoxy-2-pyridyl)piperazine-1-carbonyl]-1H-indol-6-yl]-3,6-dihydro-2H-pyridin-1-yl]-3-(triazol-1-yl)propan-1-one

1-[5-[4-(4-Ethyl-1-methyl-pyrrolidin-3-yl)-7-fluoro-2-[4-(5-fluoro-3-methoxy-2-pyridyl)piperazine-1-carbonyl]-1H-indol-6-yl]-3,6-dihydro-2H-pyridin-1-yl]-3-(triazol-1-yl)propan-1-one (80 mg, 120 μmol) was purified by SFC (column: DAICEL CHIRALPAK IK(250 mm*50 mm,10 um);mobile phase: [Hexane-ETOH:ACN(4:1)(0.1IPAM)];B %:45%, isocratic elution mode) to give the first eluting isomer as 1-[5-[4-(4-ethyl-1-methyl-pyrrolidin-3-yl)-7-fluoro-2-[4-(5-fluoro-3-methoxy-2-pyridyl)piperazine-1-carbonyl]-1H-indol-6-yl]-3,6-dihydro-2H-pyridin-1-yl]-3-(triazol-1-yl)propan-1-one (28.33 mg, 34% yield) as a white solid (. LC-MS (ESI⁺) m/z 688.4 (M+1); ¹H NMR (400 MHz, DMSO-d₆) δ=12.03 (br s, 1H), 8.16-8.06 (m, 1H), 7.80 (d, J=2.4 Hz, 1H), 7.72-7.67 (m, 1H), 7.38 (dd, J=2.4, 10.4 Hz, 1H), 7.11 (br s, 1H), 6.93-6.82 (m, 1H), 6.05 (br s, 1H), 4.62 (q, J=6.4 Hz, 2H), 4.36-4.26 (m, 2H), 3.86 (s, 3H), 3.82 (br s, 4H), 3.66-3.57 (m, 2H), 3.22 (br s, 2H), 3.10-3.11 (m, 2H), 3.01 (br s, 1H), 2.78 (br d, J=7.2 Hz, 2H), 2.32 (br s, 5H), 2.27-2.18 (m, 4H), 1.74-1.55 (m, 1H), 1.47-1.36 (m, 2H), 0.77 (t, J=7.2 Hz, 3H)) and the second eluting isomer as 1-[5-[4-(4-ethyl-1-methyl-pyrrolidin-3-yl)-7-fluoro-2-[4-(5-fluoro-3-methoxy-2-pyridyl)piperazine-1-carbonyl]-1H-indol-6-yl]-3,6-dihydro-2H-pyridin-1-yl]-3-(triazol-1-yl)propan-1-one (29 mg, 34% yield) as a white solid (688.4 (M+1)⁺; ¹H NMR (400 MHz, DMSO-d₆) δ=12.03 (br s, 1H1), 8.17-8.06 (m, 1H), 7.80 (d, J=2.4 Hz, 1H), 7.71-7.66 (m, 1H), 7.38 (dd, J=2.4, 10.4 Hz, 1H), 7.11 (br s, 1H), 6.92-6.81 (m, 1H), 6.05 (br s, 1H), 4.62 (q, J=6.4 Hz, 2H), 4.34-4.26 (m, 2H), 3.86 (s, 3H), 3.82 (br s, 4H), 3.64-3.58 (m, 2H), 3.22 (br s, 2H), 3.12-3.07 (m, 2H), 3.03-2.99 (m, 1H), 2.77 (br d, J=7.2 Hz, 2H), 2.36-2.29 (m, 5H), 2.28-2.13 (m, 4H), 1.71-1.59 (m, 1H), 1.48-1.38 (m, 2H), 0.77 (t, J=7.2 Hz, 3H)). The absolute stereochemistry of the enantiomers was assigned arbitrarily.

### Example 1. Homogenous Time-Resolved Fluorescence (HTRF) Binding Assay

HTRF binding assays were performed using 0.15 nM biotinylated truncated STAT6 (123-632)-avi purified from E. coli, 1× Streptavidin-terbium (CisBio) prepared by mixing SA-Tb in PPI detection buffer (CisBio), 20 nM proprietary fluorescein-labeled probe, and test compounds in assay buffer consisting of 50 mM HEPES-Na pH 7.5, 100 mM NaCl, 1 mM EDTA, 2 mM DTT, 0.1% Tween-20 with a final volume of 20 uL. Compound stocks were dissolved at 10 mM in 100% DMSO and 11 point titration with 3 fold serial dilution was performed in white, opaque 384 well microplates. Reaction plates were incubated at room temperature for 30 minutes. Plates were centrifuged at low rpm for 5 mins, and the ratio of fluorescence intensities were measured at emission wavelengths for fluorescein acceptor (520 nm) and terbium donor (495 nm) on Envision Plate reader. % Inhibition was calculated from the 520/495 ratio generated by using proprietary positive control compound for 100% inhibition and DMSO only reactions for 0% inhibition. Data was processed and dose response curves were generated using GraphPad Prism to determine the concentration required for inhibiting 50% of the HTRF signal (IC₅₀).

The STAT6 HTRF results are shown in Table 21. The letter codes for IC₅₀ (μM) include: A (<0.1 μM); B (0.1-1 μM); C (>1-10 μM); D (>10-100 μM); and E (>100 μM or not tested).

TABLE 21
STAT6 HTRF Results

| | STAT6 HTRF: |
|---|---|
| | Average IC₅₀ |
| I-# | (μM) |
| I-1 | E |
| I-2 | E |
| I-3 | E |
| I-4 | E |
| I-5 | D |
| I-8 | A |
| I-9 | D |
| I-10 | E |
| I-11 | D |
| I-12 | B |
| I-13 | D |
| I-14 | B |
| I-15 | D |
| I-16 | E |
| I-17 | E |
| I-18 | E |
| I-19 | E |
| I-20 | E |
| I-21 | D |
| I-22 | A |
| I-23 | B |
| I-24 | D |
| I-25 | E |
| I-26 | D |
| I-27 | D |
| I-28 | D |
| I-29 | C |
| I-30 | D |
| I-31 | D |
| I-32 | D |
| I-33 | D |
| I-34 | D |
| I-35 | C |
| I-36 | D |
| I-37 | C |
| I-38 | D |
| I-39 | D |
| I-40 | D |
| I-41 | D |
| I-42 | D |
| I-43 | D |
| I-44 | D |
| I-45 | D |
| I-46 | D |
| I-47 | D |
| I-48 | D |
| I-49 | D |
| I-50 | C |
| I-51 | D |
| I-52 | D |
| I-53 | B |
| I-54 | D |
| I-55 | C |
| I-56 | D |
| I-57 | D |
| I-58 | D |
| I-59 | D |
| I-60 | D |
| I-61 | D |
| I-62 | D |
| I-63 | D |
| I-64 | D |
| I-65 | D |
| I-66 | D |
| I-67 | D |
| I-68 | D |
| I-69 | D |
| I-70 | D |
| I-71 | D |
| I-72 | D |
| I-73 | D |
| I-74 | D |
| I-75 | D |
| I-76 | D |
| I-77 | D |
| I-78 | D |
| I-79 | D |
| I-80 | D |
| I-81 | D |
| I-82 | D |
| I-83 | C |
| I-84 | C |
| I-85 | D |
| I-86 | D |
| I-87 | B |
| I-88 | D |
| I-89 | D |
| I-90 | D |
| I-91 | C |
| I-92 | D |
| I-93 | D |
| I-94 | D |
| I-95 | D |
| I-96 | D |
| I-97 | D |
| I-98 | D |
| I-99 | D |
| I-100 | D |
| I-101 | D |
| I-102 | D |
| I-103 | D |
| I-104 | D |
| I-105 | D |
| I-106 | C |
| I-107 | D |
| I-108 | D |
| I-109 | D |
| I-110 | C |
| I-111 | D |
| I-112 | E |
| I-113 | A |
| I-114 | A |
| I-115 | B |
| I-116 | A |
| I-117 | A |
| I-118 | A |
| I-119 | D |
| I-120 | E |
| I-122 | C |
| I-123 | B |
| I-124 | C |
| I-125 | C |
| I-126 | C |
| I-127 | B |
| I-128 | B |
| I-129 | D |
| I-130 | D |
| I-132 | D |
| I-133 | C |
| I-134 | D |
| I-135 | D |
| I-136 | D |
| I-137 | D |
| I-138 | C |
| I-139 | D |
| I-140 | D |
| I-141 | D |
| I-142 | C |
| I-143 | D |
| I-144 | B |
| I-145 | C |
| I-146 | D |
| I-147 | B |
| I-148 | D |
| I-149 | D |
| I-150 | D |
| I-151 | D |
| I-152 | C |
| I-153 | D |
| I-154 | D |
| I-155 | D |
| I-156 | D |
| I-157 | C |
| I-158 | C |
| I-159 | D |
| I-160 | C |
| I-161 | D |
| I-162 | C |
| I-163 | D |
| I-165 | D |
| I-166 | A |
| I-167 | A |
| I-168 | D |
| I-169 | D |
| I-170 | D |
| I-171 | C |
| I-172 | C |
| I-173 | D |
| I-174 | D |
| I-175 | B |
| I-176 | D |
| I-177 | D |
| I-178 | D |
| I-179 | D |
| I-180 | D |
| I-181 | D |
| I-182 | D |
| I-184 | A |
| I-185 | B |
| I-186 | B |
| I-187 | D |
| I-188 | D |
| I-189 | D |
| I-190 | A |
| I-191 | D |
| I-192 | D |
| I-193 | D |
| I-194 | C |
| I-195 | D |
| I-196 | A |
| I-197 | C |
| I-198 | C |
| I-199 | D |
| I-200 | C |
| I-201 | C |
| I-202 | B |
| I-203 | B |
| I-204 | A |
| I-205 | A |
| I-206 | D |
| I-207 | A |
| I-208 | D |
| I-209 | D |
| I-210 | D |
| I-212 | C |
| I-213 | D |
| I-214 | D |
| I-215 | A |
| I-216 | D |
| I-217 | D |
| I-219 | D |
| I-220 | B |
| I-221 | D |
| I-222 | B |
| I-224 | B |
| I-225 | C |
| I-226 | D |
| I-227 | B |
| I-228 | B |
| I-229 | D |
| I-231 | D |
| I-232 | D |
| I-234 | C |
| I-235 | B |
| I-236 | C |
| I-237 | C |
| I-239 | D |
| I-241 | C |
| I-243 | C |
| I-244 | B |
| I-245 | D |
| I-246 | D |
| I-247 | D |
| I-248 | D |
| I-249 | D |
| I-251 | D |
| I-252 | C |
| I-254 | D |
| I-255 | D |
| I-256 | D |
| I-257 | D |
| I-259 | D |
| I-261 | D |
| I-263 | D |
| I-264 | D |
| I-265 | D |
| I-266 | D |
| I-267 | C |
| I-268 | C |
| I-269 | B |
| I-270 | D |
| I-271 | D |
| I-272 | D |
| I-273 | D |
| I-274 | D |
| I-275 | D |
| I-276 | C |
| I-277 | C |
| I-278 | D |
| I-279 | D |
| I-280 | D |
| I-281 | D |
| I-282 | D |
| I-283 | C |
| I-284 | D |
| I-285 | D |
| I-286 | D |
| I-287 | C |
| I-289 | D |
| I-290 | D |
| I-291 | D |
| I-292 | D |
| I-293 | D |
| I-294 | D |
| I-295 | D |
| I-296 | D |
| I-297 | D |
| I-298 | D |
| I-299 | C |
| I-300 | D |
| I-301 | C |
| I-302 | D |
| I-304 | A |
| I-305 | C |
| I-306 | D |
| I-307 | D |
| I-308 | C |
| I-309 | D |
| I-310 | C |
| I-311 | D |
| I-312 | D |
| I-313 | C |
| I-315 | D |
| I-316 | C |
| I-317 | C |
| I-318 | C |
| I-319 | C |
| I-321 | D |
| I-322 | D |
| I-323 | A |
| I-324 | A |
| I-325 | A |
| I-326 | A |
| I-327 | A |
| I-328 | A |
| I-329 | A |
| I-330 | A |
| I-331 | A |
| I-332 | A |
| I-333 | A |
| I-334 | A |
| I-335 | A |
| I-336 | A |
| I-337 | A |
| I-338 | A |
| I-339 | A |
| I-340 | A |
| I-341 | A |
| I-342 | A |
| I-343 | A |
| I-344 | A |
| I-345 | A |
| I-346 | A |
| I-347 | A |
| I-348 | A |
| I-349 | A |
| I-350 | A |
| I-351 | A |
| I-352 | A |
| I-353 | A |
| I-354 | B |
| I-355 | A |
| I-356 | A |
| I 357 | A |
| I-358 | A |
| I-359 | A |
| I-360 | A |
| I-361 | A |
| I-362 | A |
| I-363 | A |
| I-364 | A |
| I-365 | A |
| I-366 | A |
| I-367 | A |
| I-368 | A |
| I-369 | A |
| I-370 | A |
| I-371 | A |
| I-372 | A |
| I-373 | A |
| I-374 | A |
| I-375 | A |
| I-376 | C |
| I-377 | B |
| I-378 | A |
| I-379 | B |
| I-380 | A |
| I-381 | A |
| I-382 | A |
| I-383 | A |
| I-384 | A |
| I-385 | A |
| I-386 | A |
| I-387 | A |
| I-388 | A |
| I-389 | A |
| I-390 | A |
| I-391 | A |
| I-392 | A |
| I-393 | A |
| I-394 | A |
| I-395 | A |
| I-396 | A |
| I-397 | A |
| I-398 | A |
| I-399 | A |
| I-400 | A |
| I-401 | A |
| I-402 | A |
| I-403 | A |
| I-404 | A |
| I-405 | A |
| I-406 | A |
| I-407 | A |
| I-408 | A |
| I-409 | B |
| I-410 | A |
| I-411 | A |
| I-412 | A |
| I-413 | B |
| I-414 | B |
| I-415 | A |
| I-416 | A |
| I-417 | A |
| I-418 | A |
| I-419 | A |
| I-420 | B |
| I-421 | A |
| I-422 | A |
| I-423 | A |
| I-424 | A |
| I-425 | A |
| I-426 | A |
| I-427 | A |
| I-428 | A |
| I-429 | A |
| I-430 | B |
| I-431 | A |
| I-432 | A |
| I-433 | E |
| I-434 | A |
| I-435 | A |
| I-436 | A |
| I-437 | A |
| I-438 | A |
| I-439 | A |
| I-440 | A |
| I-441 | A |
| I-442 | A |
| I-443 | A |
| I-444 | A |
| I-445 | A |
| I-446 | C |
| I-447 | A |
| I-448 | A |
| I-449 | A |
| I-450 | A |
| I-451 | A |
| I-452 | E |
| I-453 | A |
| I-454 | A |
| I-455 | A |
| I-456 | A |
| I-457 | A |
| I-458 | A |
| I-459 | A |
| I-460 | A |
| I-461 | B |
| I-462 | B |
| I-463 | A |
| I-464 | A |
| I-465 | E |
| I-466 | A |
| I-467 | A |
| I-468 | A |
| I-469 | B |
| I-470 | A |
| I-471 | A |
| I-472 | A |
| I-473 | A |
| I-474 | A |
| I-475 | A |
| I-476 | A |
| I-477 | A |
| I-478 | A |
| I-479 | A |
| I-480 | A |
| I-481 | A |
| I-482 | A |
| I-483 | A |
| I-484 | E |
| I-485 | A |
| I-486 | A |
| I-487 | A |
| I-488 | A |
| I-489 | A |
| I-490 | E |
| I-491 | E |
| I-492 | E |
| I-493 | E |
| I-494 | E |
| I-495 | E |
| I-496 | E |
| I-497 | D |
| I-498 | D |
| I-499 | D |
| I-500 | E |
| I-501 | D |
| I-502 | D |
| I-503 | D |
| I-504 | D |
| I-505 | D |
| I-506 | D |
| I-507 | D |
| I-508 | A |
| I-509 | A |
| I-510 | A |
| I-511 | A |
| I-512 | A |
| I-513 | A |
| I-514 | A |
| I-515 | A |
| I-516 | A |
| I-517 | A |
| I-518 | A |
| I-519 | A |
| I-520 | A |
| I-521 | A |
| I-522 | A |
| I-523 | A |
| I-524 | A |
| I-525 | A |
| I-526 | A |
| I-527 | A |
| I-528 | B |
| I-529 | A |
| I-530 | A |
| I-531 | A |
| I-532 | A |
| I-533 | A |
| I-534 | E |
| I-535 | E |
| I-536 | A |
| I-537 | A |
| I-538 | A |
| I-539 | A |
| I-540 | A |
| I-541 | A |
| I-542 | E |
| I-543 | A |
| I-544 | A |
| I-545 | A |
| I-546 | E |
| I-547 | E |
| I-548 | E |
| I-549 | E |
| I-550 | E |
| I-551 | A |
| I-552 | B |
| I-553 | B |
| I-554 | B |
| I-555 | B |
| I-556 | A |
| I-557 | A |
| I-558 | A |
| I-559 | A |
| I-560 | A |
| I-561 | A |
| I-562 | A |
| I-563 | A |
| I-564 | A |
| I-565 | A |
| I-566 | A |
| I-567 | A |
| I-568 | A |
| I-569 | A |
| I-570 | A |
| I-571 | A |
| I-572 | A |
| I-573 | A |
| I-574 | A |
| I-575 | A |
| I-576 | A |
| I-577 | A |
| I-578 | A |
| I-579 | A |
| I-580 | A |
| I-581 | A |
| I-582 | A |
| I-583 | E |
| I-584 | A |
| I-585 | A |
| I-586 | A |
| I-587 | A |
| I-588 | A |
| I-589 | A |
| I-590 | A |
| I-591 | A |
| I-592 | A |
| I-593 | A |
| I-594 | A |
| I-595 | A |
| I-596 | A |
| I-597 | A |
| I-598 | A |
| I-599 | A |
| I-600 | A |
| I-601 | A |
| I-602 | A |
| I-603 | A |
| I-604 | A |
| I-605 | A |
| I-606 | A |
| I-607 | E |
| I-608 | E |
| I-609 | A |
| I-610 | A |
| I-611 | A |
| I-612 | A |
| I-613 | A |
| I-614 | A |
| I-615 | A |
| I-616 | E |
| I-617 | A |
| I-618 | A |
| I-619 | B |
| I-620 | A |
| I-621 | A |
| I-622 | A |
| I-623 | A |
| I-624 | A |
| I-625 | A |
| I-626 | A |
| I-627 | A |
| I-628 | A |
| I-629 | A |
| I-630 | A |
| I-631 | A |
| I-632 | A |
| I-633 | A |
| I-634 | A |
| I-635 | A |
| I-636 | A |
| I-637 | A |
| I-638 | A |
| I-639 | A |
| I-640 | A |
| I-641 | A |
| I-642 | B |
| I-643 | A |
| I-644 | A |
| I-645 | A |
| I-646 | A |
| I-647 | A |
| I-648 | A |
| I-649 | A |
| I-650 | A |
| I-651 | A |
| I-652 | A |
| I-653 | A |
| I-654 | A |
| I-655 | A |
| I-656 | A |
| I-657 | A |
| I-658 | A |
| I-659 | A |
| I-660 | A |
| I-661 | A |
| I-662 | A |
| I-663 | A |
| I-664 | A |
| I-665 | A |
| I-666 | E |
| I-667 | E |
| I-668 | E |
| I-669 | A |
| I-670 | A |
| I-671 | B |
| I-672 | A |
| I-673 | A |
| I-674 | B |
| I-675 | A |
| I-676 | B |
| I-677 | A |
| I-678 | A |
| I-679 | A |
| I-680 | A |
| I-681 | A |
| I-682 | A |
| I-683 | A |
| I-684 | A |
| I-685 | A |
| I-686 | A |
| I-687 | A |
| I-688 | A |
| I-689 | A |
| I-690 | A |
| I-691 | A |
| I-692 | E |
| I-693 | E |
| I-694 | E |
| I-695 | E |
| I-696 | E |
| I-697 | E |
| I-698 | D |
| I-699 | D |
| I-700 | D |
| I-701 | D |
| I-702 | D |
| I-703 | D |
| I-704 | D |
| I-705 | D |
| I-706 | D |
| I-707 | D |
| I-708 | D |
| I-709 | D |
| I-710 | D |
| I-711 | D |
| I-712 | D |
| I-713 | D |
| I-714 | D |
| I-715 | D |
| I-716 | D |
| I-717 | D |
| I-718 | D |
| I-719 | D |
| I-720 | D |
| I-721 | D |
| I-722 | D |
| I-723 | D |
| I-724 | A |
| I-725 | A |
| I-726 | A |
| I-727 | A |
| I-728 | E |
| I-729 | A |
| I-730 | A |
| I-731 | A |
| I-732 | E |
| I-733 | A |
| I-734 | A |
| I-735 | A |
| I-736 | A |
| I-737 | A |
| I-738 | A |
| I-739 | A |
| I-740 | A |
| I-741 | B |
| I-742 | A |
| I-743 | A |
| I-744 | A |
| I-745 | A |
| I-746 | A |
| I-747 | A |
| I-748 | A |
| I-749 | A |
| I-750 | E |
| I-751 | A |
| I-752 | A |
| I-753 | A |
| I-754 | A |
| I-755 | A |
| I-756 | A |
| I-757 | A |
| I-758 | A |
| I-759 | A |
| I-760 | E |
| I-761 | E |
| I-762 | E |
| I-763 | A |
| I-764 | A |
| I-765 | A |
| I-766 | E |
| I-767 | E |
| I-768 | E |
| I-769 | A |
| I-770 | C |
| I-771 | A |
| I-772 | E |
| I-773 | A |
| I-774 | A |
| I-775 | A |
| I-776 | A |
| I-777 | A |
| I-778 | A |
| I-779 | A |
| I-780 | A |
| I-781 | A |
| I-782 | A |
| I-783 | A |
| I-784 | A |
| I-785 | A |
| I-786 | A |
| I-787 | A |
| I-788 | A |
| I-789 | A |
| I-790 | A |
| I-791 | A |
| I-792 | A |
| I-793 | A |
| I-794 | A |
| I-795 | E |
| I-796 | E |
| I-797 | E |
| I-798 | E |
| I-799 | E |
| I-800 | E |
| I-801 | E |
| I-802 | B |
| I-803 | D |
| I-804 | B |
| I-805 | D |
| I-806 | D |
| I-807 | A |
| I-808 | B |
| I-809 | E |
| I-810 | E |
| I-811 | E |
| I-812 | E |
| I-813 | E |
| I-814 | E |
| I-815 | B |
| I-816 | B |
| I-817 | B |
| I-818 | B |
| I-819 | B |
| I-820 | B |
| I-821 | B |
| I-822 | B |
| I-823 | A |
| I-824 | B |
| I-825 | B |
| I-826 | B |
| I-827 | B |
| I-828 | B |
| I-829 | B |
| I-830 | B |
| I-831 | B |
| I-832 | C |
| I-833 | B |
| I-834 | B |
| I-835 | B |
| I-837 | B |
| I-838 | A |
| I-839 | A |
| I-840 | A |
| I-841 | A |
| I-842 | A |
| I-843 | A |
| I-844 | B |
| I-845 | A |
| I-846 | A |
| I-847 | B |
| I-848 | A |
| I-849 | A |
| I-850 | A |
| I-851 | A |
| I-852 | B |
| I-853 | A |
| I-854 | A |
| I-855 | A |
| I-856 | A |
| I-857 | B |
| I-858 | A |
| I-859 | A |
| I-860 | B |
| I-861 | A |
| I-862 | A |
| I-863 | A |
| I-864 | A |
| I-865 | A |
| I-866 | A |
| I-867 | A |
| I-868 | B |
| I-869 | A |
| I-870 | A |
| I-871 | A |
| I-872 | B |
| I-873 | A |
| I-874 | B |
| I-875 | A |
| I-876 | B |
| I-877 | B |
| I-878 | A |
| I-879 | A |
| I-880 | B |
| I-881 | B |
| I-882 | B |
| I-883 | B |
| I-884 | B |
| I-885 | B |
| I-886 | B |
| I-887 | B |
| I-888 | B |
| I-889 | A |
| I-890 | B |
| I-891 | B |
| I-892 | A |
| I-893 | A |
| I-894 | B |
| I-895 | A |
| I-896 | B |
| I-897 | B |
| I-898 | A |
| I-899 | A |
| I-900 | A |
| I-901 | A |
| I-902 | C |
| I-903 | B |
| I-904 | B |
| I-905 | A |
| I-906 | A |
| I-907 | A |
| I-908 | A |
| I-909 | A |
| I-910 | A |
| I-911 | B |
| I-912 | B |
| I-913 | B |
| I-914 | A |
| I-915 | A |
| I-916 | B |
| I-917 | A |
| I-918 | A |
| I-919 | A |
| I-920 | A |
| I-921 | B |
| I-922 | B |
| I-923 | A |
| I-924 | A |
| I-925 | B |
| I-926 | A |
| I-927 | B |
| I-928 | A |
| I-929 | A |
| I-930 | B |
| I-931 | B |
| I-932 | B |
| I-933 | A |
| I-934 | A |
| I-935 | A |
| I-936 | A |
| I-937 | B |
| I-938 | A |
| I-939 | A |
| I-940 | A |
| I-941 | A |
| I-942 | A |
| I-943 | A |
| I-944 | A |
| I-945 | B |
| I-946 | A |
| I-947 | C |
| I-948 | A |
| I-949 | A |
| I-950 | A |
| I-951 | A |
| I-952 | A |
| I-953 | A |
| I-954 | A |
| I-955 | A |
| I-956 | C |
| I-957 | B |
| I-958 | B |
| I-959 | A |
| I-960 | A |
| I-961 | A |
| I-962 | A |
| I-963 | A |
| I-964 | B |
| I-965 | B |
| I-966 | A |
| I-967 | B |
| I-968 | B |
| I-969 | B |
| I-970 | A |
| I-971 | A |
| I-972 | A |
| I-973 | B |
| I-974 | A |
| I-975 | A |
| I-976 | A |
| I-977 | A |
| I-978 | B |
| I-979 | B |
| I-980 | A |
| I-981 | B |
| I-982 | E |
| I-983 | A |
| I-984 | A |
| I-985 | A |
| I-986 | A |
| I-987 | C |
| I-988 | A |
| I-989 | A |
| I-990 | A |
| I-991 | A |
| I-992 | A |
| I-993 | B |
| I-994 | A |
| I-995 | A |
| I-996 | A |
| I-997 | A |
| I-998 | A |
| I-999 | A |
| I-1000 | A |
| I-1001 | A |
| I-1002 | A |
| I-1003 | A |
| I-1004 | A |
| I-1005 | A |
| I-1006 | B |
| I-1007 | A |
| I-1008 | A |
| I-1009 | A |
| I-1010 | A |
| I-1011 | A |
| I-1012 | A |
| I-1013 | A |
| I-1014 | A |
| I-1015 | A |
| I-1016 | A |
| I-1017 | A |
| I-1018 | A |
| I-1019 | A |
| I-1020 | A |
| I-1021 | A |
| I-1022 | A |
| I-1023 | A |
| I-1024 | A |
| I-1025 | A |
| I-1026 | A |
| I-1027 | B |
| I-1028 | C |
| I-1029 | A |
| I-1030 | A |
| I-1031 | A |
| I-1032 | B |
| I-1033 | A |
| I-1034 | A |
| I-1035 | B |
| I-1036 | A |
| I-1037 | B |
| I-1038 | B |
| I-1039 | A |
| I-1040 | A |
| I-1041 | A |
| I-1042 | D |
| I-1043 | E |
| I-1044 | C |
| I-1045 | B |
| I-1046 | B |
| I-1047 | B |
| I-1048 | A |
| I-1049 | B |
| I-1050 | A |
| I-1051 | E |
| I-1052 | A |
| I-1053 | A |
| I-1054 | A |
| I-1055 | A |
| I-1056 | A |
| I-1057 | A |
| I-1058 | A |
| I-1059 | A |
| I-1060 | A |
| I-1061 | A |
| I-1062 | A |
| I-1063 | A |
| I-1064 | A |
| I-1065 | A |
| I-1066 | A |
| I-1067 | A |
| I-1068 | A |
| I-1069 | A |
| I-1070 | A |
| I-1071 | A |
| I-1072 | A |
| I-1073 | A |
| I-1074 | A |
| I-1075 | A |
| I-1076 | A |
| I-1077 | A |
| I-1078 | A |
| I-1079 | A |
| I-1080 | A |
| I-1081 | A |
| I-1082 | A |
| I-1083 | A |
| I-1084 | A |
| I-1085 | A |
| I-1086 | A |
| I-1087 | A |
| I-1088 | A |
| I-1089 | A |
| I-1090 | A |
| I-1091 | B |
| I-1092 | A |
| I-1093 | B |
| I-1094 | A |
| I-1095 | A |
| I-1096 | A |
| I-1097 | E |
| I-1098 | A |
| I-1099 | B |
| I-1100 | A |
| I-1101 | A |
| I-1103 | A |
| I-1104 | B |
| I-1105 | B |
| I-1106 | C |
| I-1107 | B |
| I-1108 | A |
| I-1109 | A |
| I-1110 | A |
| I-1111 | A |
| I-1112 | A |
| I-1113 | B |
| I-1114 | A |
| I-1115 | A |
| I-1116 | A |
| I-1117 | A |
| I-1118 | A |
| I-1119 | A |
| I-1120 | A |
| I-1121 | A |
| I-1122 | A |
| I-1123 | A |
| I-1124 | B |
| I-1125 | A |
| I-1126 | A |
| I-1127 | B |
| I-1128 | A |
| I-1129 | A |
| I-1130 | B |
| I-1131 | A |
| I-1132 | A |
| I-1133 | A |
| I-1134 | A |
| I-1135 | A |
| I-1136 | A |
| I-1137 | A |
| I-1138 | A |
| I-1139 | A |
| I-1140 | A |
| I-1141 | A |
| I-1142 | A |
| I-1143 | A |
| I-1144 | A |
| I-1145 | A |
| I-1146 | A |
| I-1147 | A |
| I-1148 | A |
| I-1149 | A |
| I-1150 | A |
| I-1151 | A |
| I-1152 | A |
| I-1153 | A |
| I-1154 | A |
| I-1155 | A |
| I-1156 | A |
| I-1157 | A |
| I-1158 | A |
| I-1159 | B |
| I-1160 | A |
| I-1161 | B |
| I-1162 | C |
| I-1163 | B |
| I-1164 | B |
| I-1165 | A |
| I-1166 | A |
| I-1167 | A |
| I-1168 | B |
| I-1169 | B |
| I-1170 | B |
| I-1171 | B |
| I-1172 | B |
| I-1173 | B |
| I-1174 | C |
| I-1175 | B |
| I-1176 | A |
| I-1177 | B |
| I-1178 | B |
| I-1179 | A |
| I-1180 | A |
| I-1181 | A |
| I-1182 | A |
| I-1183 | A |
| I-1184 | A |
| I-1185 | A |
| I-1186 | A |
| I-1187 | A |
| I-1188 | B |
| I-1189 | B |
| I-1190 | B |
| I-1191 | C |
| I-1192 | C |
| I-1193 | A |
| I-1194 | A |
| I-1195 | E |
| I-1196 | A |
| I-1197 | B |
| I-1198 | A |
| I-1199 | B |
| I-1200 | A |
| I-1201 | A |
| I-1202 | A |
| I-1203 | C |
| I-1204 | C |
| I-1205 | C |
| I-1206 | A |
| I-1207 | A |
| I-1208 | A |
| I-1209 | A |
| I-1210 | A |
| I-1211 | A |
| I-1212 | A |
| I-1213 | E |
| I-1214 | A |
| I-1215 | A |
| I-1216 | E |
| I-1217 | A |
| I-1218 | A |
| I-1219 | E |
| I-1220 | A |
| I-1221 | A |
| I-1222 | A |
| I-1223 | A |
| I-1224 | C |
| I-1225 | C |
| I-1226 | C |
| I-1227 | A |
| I-1228 | A |
| I-1229 | E |
| I-1230 | C |
| I-1231 | A |
| I-1232 | B |
| I-1233 | A |
| I-1234 | B |
| I-1235 | C |
| I-1236 | A |
| I-1237 | B |
| I-1238 | C |
| I-1239 | C |
| I-1241 | C |
| I-1242 | C |
| I-1243 | C |
| I-1244 | C |
| I-1245 | C |
| I-1247 | B |
| I-1248 | C |
| I-1249 | C |
| I-1250 | A |
| I-1251 | D |
| I-1252 | D |
| I-1253 | D |
| I-1254 | D |
| I-1255 | D |
| I-1256 | D |
| I-1257 | D |
| I-1258 | D |
| I-1259 | D |
| I-1261 | D |
| I-1262 | D |
| I-1263 | D |
| I-1264 | D |
| I-1265 | D |
| I-1266 | D |
| I-1267 | D |
| I-1268 | D |
| I-1269 | D |
| I-1270 | D |
| I-1274 | D |
| I-1275 | D |
| I-1276 | D |
| I-1277 | D |
| I-1278 | D |
| I-1279 | D |
| I-1280 | D |
| I-1281 | D |
| I-1282 | D |
| I-1283 | D |
| I-1284 | D |
| I-1285 | D |
| I-1286 | D |
| I-1287 | D |
| I-1288 | D |
| I-1289 | D |
| I-1290 | D |
| I-1291 | D |
| I-1292 | D |
| I-1299 | D |
| I-1300 | D |
| I-1301 | D |
| I-1302 | D |
| I-1303 | D |
| I-1304 | D |
| I-1305 | D |
| I-1306 | D |
| I-1307 | D |
| I-1308 | D |
| I-1309 | D |
| I-1310 | D |
| I-1311 | D |
| I-1312 | D |
| I-1313 | D |
| I-1314 | D |
| I-1315 | D |
| I-1316 | D |
| I-1317 | D |
| I-1318 | D |
| I-1319 | D |
| I-1320 | D |
| I-1321 | D |
| I-1322 | D |
| I-1324 | C |
| I-1326 | A |
| I-1327 | B |
| I-1328 | B |
| I-1329 | C |
| I-1330 | A |
| I-1331 | B |
| I-1332 | A |
| I-1333 | C |
| I-1334 | D |
| I-1335 | C |
| I-1336 | C |
| I-1337 | C |
| I-1338 | C |
| I-1339 | C |
| I-1340 | B |
| I-1341 | A |
| I-1342 | A |
| I-1343 | A |
| I-1344 | C |
| I-1345 | C |
| I-1346 | A |
| I-1347 | B |
| I-1348 | B |
| I-1349 | B |
| I-1350 | A |
| I-1351 | A |
| I-1352 | B |
| I-1353 | B |
| I-1354 | B |
| I-1355 | A |
| I-1356 | D |
| I-1357 | A |
| I-1358 | A |
| I-1359 | B |
| I-1360 | A |
| I-1361 | A |
| I-1362 | A |
| I-1363 | A |
| I-1364 | B |
| I-1365 | A |
| I-1366 | A |
| I-1367 | B |
| I-1368 | A |
| I-1369 | A |
| I-1370 | A |
| I-1371 | A |
| I-1372 | A |
| I-1373 | A |
| I-1374 | A |
| I-1375 | B |
| I-1376 | A |
| I-1377 | B |
| I-1378 | A |
| I-1379 | A |
| I-1380 | A |
| I-1381 | A |
| I-1382 | B |
| I-1383 | B |
| I-1384 | A |
| I-1385 | A |
| I-1386 | A |
| I-1387 | B |
| I-1388 | A |
| I-1389 | B |
| I-1390 | A |
| I-1391 | A |
| I-1392 | A |
| I-1393 | B |
| I-1394 | A |
| I-1395 | A |
| I-1396 | B |
| I-1397 | A |
| I-1398 | A |
| I-1399 | B |
| I-1400 | A |
| I-1401 | A |
| I-1402 | B |
| I-1403 | A |
| I-1404 | A |
| I-1405 | A |
| I-1406 | A |
| I-1407 | A |
| I-1408 | A |
| I-1409 | B |
| I-1410 | B |
| I-1411 | B |
| I-1412 | A |
| I-1413 | A |
| I-1414 | B |
| I-1415 | A |
| I-1416 | A |
| I-1417 | B |
| I-1418 | A |
| I-1419 | D |
| I-1420 | B |
| I-1421 | A |
| I-1422 | C |
| I-1423 | A |
| I-1424 | A |
| I-1425 | A |
| I-1426 | A |
| I-1427 | A |
| I-1428 | A |
| I-1429 | A |
| I-1430 | A |
| I-1431 | A |
| I-1432 | B |
| I-1433 | A |
| I-1434 | B |
| I-1435 | A |
| I-1436 | B |
| I-1437 | A |
| I-1438 | A |
| I-1439 | B |
| I-1440 | A |
| I-1441 | A |
| I-1442 | A |
| I-1443 | A |
| I-1444 | A |
| I-1445 | A |
| I-1446 | A |
| I-1447 | A |
| I-1448 | A |
| I-1449 | A |
| I-1450 | A |
| I-1451 | A |
| I-1452 | B |
| I-1453 | A |
| I-1454 | C |
| I-1455 | A |
| I-1456 | A |
| I-1457 | B |
| I-1458 | C |
| I-1459 | A |
| I-1460 | A |
| I-1461 | A |
| I-1462 | A |
| I-1463 | A |
| I-1464 | B |
| I-1465 | A |
| I-1466 | A |
| I-1467 | A |
| I-1468 | A |
| I-1469 | A |
| I-1470 | A |
| I-1471 | A |
| I-1472 | B |
| I-1473 | B |
| I-1474 | A |
| I-1475 | B |
| I-1476 | C |
| I-1477 | A |
| I-1478 | A |
| I-1479 | B |
| I-1480 | A |
| I-1481 | A |
| I-1482 | B |
| I-1483 | A |
| I-1484 | A |
| I-1485 | A |
| I-1486 | A |
| I-1487 | A |
| I-1488 | C |
| I-1489 | A |
| I-1490 | A |
| I-1491 | A |
| I-1492 | A |
| I-1493 | B |
| I-1494 | B |
| I-1495 | B |
| I-1496 | B |
| I-1497 | B |
| I-1498 | B |
| I-1499 | A |
| I-1500 | B |
| I-1501 | B |
| I-1502 | B |
| I-1503 | B |
| I-1504 | B |
| I-1505 | B |
| I-1506 | B |
| I-1507 | B |
| I-1508 | A |
| I-1509 | B |
| I-1510 | A |
| I-1511 | A |
| I-1512 | C |
| I-1513 | B |
| I-1514 | B |
| I-1515 | B |
| I-1516 | B |
| I-1517 | B |
| I-1518 | B |
| I-1519 | A |
| I-1520 | A |
| I-1521 | A |
| I-1522 | B |
| I-1523 | C |
| I-1524 | C |
| I-1525 | B |
| I-1526 | C |
| I-1527 | A |
| I-1528 | A |
| I-1529 | C |
| I-1530 | A |
| I-1531 | A |
| I-1532 | B |
| I-1533 | B |
| I-1534 | A |
| I-1535 | B |
| I-1536 | A |
| I-1537 | C |
| I-1538 | A |
| I-1539 | B |
| I-1540 | A |
| I-1541 | C |
| I-1542 | C |
| I-1543 | C |
| I-1544 | A |
| I-1545 | B |
| I-1546 | A |
| I-1547 | B |
| I-1548 | A |
| I-1549 | C |
| I-1550 | B |
| I-1551 | A |
| I-1552 | A |
| I-1553 | B |
| I-1554 | C |
| I-1555 | B |
| I-1556 | C |
| I-1557 | C |
| I-1558 | A |
| I-1559 | B |
| I-1560 | A |
| I-1561 | B |
| I-1562 | B |
| I-1563 | A |
| I-1564 | B |
| I-1565 | A |
| I-1566 | B |
| I-1567 | A |
| I-1568 | B |
| I-1569 | A |
| I-1570 | C |
| I-1571 | A |
| I-1572 | B |
| I-1573 | C |
| I-1574 | A |
| I-1575 | C |
| I-1576 | A |
| I-1577 | C |
| I-1578 | A |
| I-1579 | B |
| I-1580 | B |
| I-1581 | A |
| I-1582 | B |
| I-1584 | B |
| I-1585 | A |
| I-1586 | A |
| I-1587 | B |
| I-1588 | C |
| I-1589 | B |
| I-1590 | B |
| I-1591 | B |
| I-1592 | C |
| I-1593 | B |
| I-1594 | B |
| I-1595 | B |
| I-1596 | B |
| I-1597 | C |
| I-1598 | A |
| I-1600 | B |
| I-1601 | C |
| I-1602 | C |
| I-1603 | B |
| I-1604 | C |
| I-1605 | C |
| I-1606 | C |
| I-1607 | A |
| I-1608 | B |
| I-1609 | A |
| I-1610 | C |
| I-1611 | C |
| I-1612 | B |
| I-1613 | B |
| I-1614 | A |
| I-1615 | B |
| I-1616 | A |
| I-1617 | C |
| I-1618 | C |
| I-1619 | C |
| I-1620 | C |
| I-1621 | A |
| I-1622 | A |
| I-1623 | A |
| I-1624 | A |
| I-1626 | A |
| I-1627 | A |
| I-1628 | C |
| I-1629 | C |
| I-1630 | A |
| I-1631 | A |
| I-1632 | A |
| I-1633 | C |
| I-1634 | B |
| I-1635 | C |
| I-1636 | B |
| I-1637 | A |
| I-1638 | A |
| I-1639 | A |
| I-1640 | A |
| I-1641 | A |
| I-1642 | A |
| I-1643 | A |
| I-1644 | C |
| I-1645 | A |
| I-1646 | A |
| I-1647 | A |
| I-1648 | C |
| I-1649 | A |
| I-1650 | C |
| I-1651 | C |
| I-1652 | C |
| I-1653 | A |
| I-1654 | A |
| I-1655 | A |
| I-1656 | B |
| I-1657 | A |
| I-1658 | B |
| I-1659 | B |
| I-1660 | C |
| I-1661 | C |
| I-1662 | C |
| I-1663 | C |
| I-1664 | A |
| I-1665 | A |
| I-1666 | A |
| I-1667 | C |
| I-1668 | A |
| I-1669 | A |
| I-1670 | B |
| I-1671 | A |
| I-1672 | A |
| I-1673 | B |
| I-1674 | D |
| I-1675 | A |
| I-1676 | A |
| I-1677 | A |
| I-1678 | A |
| I-1679 | A |
| I-1680 | A |
| I-1681 | C |
| I-1682 | B |
| I-1683 | C |
| I-1684 | C |
| I-1685 | A |

### Example 2. Phosphorylated STAT6 (pSTAT6) Flow Cytometry Assay in Human PBMC

Phosphorylation of STAT6 in human PBMC was quantitatively measured using flow cytometry technology. Human PBMC were seeded in 96-well plates with a density of 0.3 to 0.5 million cells per well in 100 μl fresh media. Compounds were then added to the assay plates with a final top concentration of up to 30 μM in a 1:3 dilution series with total of 9 doses. The assay plates were incubated for 1 hour at 37° C. under 5% CO₂. The cells were then stimulated with 1 ng/ml of IL-4 (R&D Systems, Cat #6507-IL) for 15 minutes, followed by fixation for 30 minutes at 4° C. Cells were spun down at 350 g for 5 minutes. The supernatant was removed and cells were washed once with FACS buffer. Cells were permeabilized with 90% MeOH/H₂O for 30 minutes at 4° C., followed by one wash with FACS buffer. Non-specific binding were blocked with human Fc for 1 hour at 4 C, followed by one wash with FACS buffer. AF488 conjugated pSTAT6 antibody (BD Biosciences, Cat #612600, 1:100 dilution) and PE conjugated CD19 antibody (BD Biosciences, Cat #341103, 1:50 dilution) diluted in 100 μl FACS buffer was added to each well with overnight incubation at 4° C. Cells were then washed once with FACS buffer and resuspended in 200 μl of FACS buffer for flow cytometry to quantify pSTAT6 in CD19+ B cells. The data was analyzed by GraphPad Prism and the dose dependent pSTAT6 inhibition was fit using the four-parameter inhibitor vs. response nonlinear regression.

The STAT6 IL4 pSTAT6 results are shown in Table 22. The letter codes for IC₅₀ (μM) include: A (<0.1 μM); B (0.1-1 μM); C (>1-10 μM); D (>10-100 μM); and E (>100 μM or not tested).

TABLE 22
STAT6 IL4 pSTAT6 Results

| | STAT6 IL4 |
|---|---|
| | pSTAT6 assay |
| | in PBMC: |
| | Average IC₅₀ |
| I-# | (μM) |
| I-1 | E |
| I-2 | E |
| I-3 | E |
| I-4 | E |
| I-5 | D |
| I-8 | B |
| I-9 | E |
| I-10 | E |
| I-11 | D |
| I-12 | D |
| I-13 | B |
| I-14 | D |
| I-15 | D |
| I-16 | E |
| I-17 | E |
| I-18 | E |
| I-19 | E |
| I-20 | E |
| I-21 | E |
| I-22 | B |
| I-23 | E |
| I-24 | D |
| I-25 | E |
| I-26 | E |
| I-27 | E |
| I-28 | D |
| I-29 | D |
| I-30 | D |
| I-31 | E |
| I-32 | D |
| I-33 | D |
| I-34 | D |
| I-35 | D |
| I-36 | D |
| I-37 | D |
| I-38 | E |
| I-39 | D |
| I-40 | D |
| I-41 | D |
| I-42 | D |
| I-43 | E |
| I-44 | D |
| I-45 | D |
| I-46 | D |
| I-47 | D |
| I-48 | D |
| I-49 | D |
| I-50 | D |
| I-51 | D |
| I-52 | D |
| I-53 | C |
| I-54 | D |
| I-55 | D |
| I-56 | D |
| I-57 | D |
| I-58 | E |
| I-59 | C |
| I-60 | E |
| I-61 | E |
| I-62 | D |
| I-63 | E |
| I-64 | E |
| I-65 | E |
| I-66 | C |
| I-67 | D |
| I-68 | D |
| I-69 | E |
| I-70 | E |
| I-71 | D |
| I-72 | E |
| I-73 | D |
| I-74 | D |
| I-75 | D |
| I-76 | E |
| I-77 | D |
| I-78 | E |
| I-79 | D |
| I-80 | E |
| I-81 | E |
| I-82 | D |
| I-83 | D |
| I-84 | E |
| I-85 | E |
| I-86 | E |
| I-87 | D |
| I-88 | D |
| I-89 | E |
| I-90 | D |
| I-91 | D |
| I-92 | D |
| I-93 | D |
| I-94 | D |
| I-95 | E |
| I-96 | D |
| I-97 | D |
| I-98 | D |
| I-99 | E |
| I-100 | D |
| I-101 | E |
| I-102 | D |
| I-103 | E |
| I-104 | E |
| I-105 | D |
| I-106 | C |
| I-107 | B |
| I-108 | E |
| I-109 | D |
| I-110 | E |
| I-111 | E |
| I-112 | E |
| I-113 | A |
| I-114 | B |
| I-115 | C |
| I-116 | B |
| I-117 | A |
| I-118 | A |
| I-119 | E |
| I-120 | E |
| I-122 | E |
| I-123 | D |
| I-124 | E |
| I-125 | E |
| I-126 | E |
| I-127 | D |
| I-128 | D |
| I-129 | E |
| I-130 | E |
| I-132 | E |
| I-133 | E |
| I-134 | E |
| I-135 | E |
| I-136 | E |
| I-137 | E |
| I-138 | E |
| I-139 | E |
| I-140 | E |
| I-141 | E |
| I-142 | E |
| I-143 | E |
| I-144 | E |
| I-145 | E |
| I-146 | E |
| I-147 | E |
| I-148 | E |
| I-149 | E |
| I-150 | E |
| I-151 | E |
| I-152 | E |
| I-153 | E |
| I-154 | E |
| I-155 | E |
| I-156 | E |
| I-157 | E |
| I-158 | E |
| I-159 | E |
| I-160 | E |
| I-161 | E |
| I-162 | E |
| I-163 | E |
| I-165 | E |
| I-166 | E |
| I-167 | E |
| I-168 | E |
| I-169 | E |
| I-170 | E |
| I-171 | D |
| I-172 | E |
| I-173 | E |
| I-174 | E |
| I-175 | C |
| I-176 | E |
| I-177 | E |
| I-178 | E |
| I-179 | E |
| I-180 | E |
| I-181 | E |
| I-182 | E |
| I-184 | D |
| I-185 | D |
| I-186 | E |
| I-187 | E |
| I-188 | E |
| I-189 | E |
| I-190 | D |
| I-191 | E |
| I-192 | E |
| I-193 | E |
| I-194 | E |
| I-195 | E |
| I-196 | D |
| I-197 | D |
| I-198 | E |
| I-199 | E |
| I-200 | E |
| I-201 | E |
| I-202 | B |
| I-203 | D |
| I-204 | E |
| I-205 | B |
| I-206 | E |
| I-207 | E |
| I-208 | E |
| I-209 | E |
| I-210 | E |
| I-212 | D |
| I-213 | E |
| I-214 | E |
| I-215 | D |
| I-216 | E |
| I-217 | E |
| I-219 | E |
| I-220 | E |
| I-221 | E |
| I-222 | D |
| I-224 | D |
| I-225 | E |
| I-226 | E |
| I-227 | D |
| I-228 | D |
| I-229 | E |
| I-231 | E |
| I-232 | E |
| I-234 | E |
| I-235 | E |
| I-236 | E |
| I-237 | E |
| I-239 | E |
| I-241 | E |
| I-243 | E |
| I-244 | E |
| I-245 | E |
| I-246 | E |
| I-247 | E |
| I-248 | E |
| I-249 | E |
| I-251 | E |
| I-252 | E |
| I-254 | E |
| I-255 | E |
| I-256 | E |
| I-257 | E |
| I-259 | E |
| I-261 | E |
| I-263 | E |
| I-264 | E |
| I-265 | E |
| I-266 | E |
| I-267 | E |
| I-268 | E |
| I-269 | D |
| I-270 | E |
| I-271 | E |
| I-272 | E |
| I-273 | E |
| I-274 | E |
| I-275 | E |
| I-276 | E |
| I-277 | E |
| I-278 | E |
| I-279 | E |
| I-280 | E |
| I-281 | E |
| I-282 | E |
| I-283 | E |
| I-284 | E |
| I-285 | E |
| I-286 | E |
| I-287 | E |
| I-289 | E |
| I-290 | E |
| I-291 | E |
| I-292 | E |
| I-293 | E |
| I-294 | E |
| I-295 | E |
| I-296 | E |
| I-297 | E |
| I-298 | E |
| I-299 | E |
| I-300 | E |
| I-301 | E |
| I-302 | E |
| I-304 | A |
| I-305 | E |
| I-306 | E |
| I-307 | E |
| I-308 | E |
| I-309 | E |
| I-310 | E |
| I-311 | E |
| I-312 | E |
| I-313 | E |
| I-315 | E |
| I-316 | E |
| I-317 | E |
| I-318 | E |
| I-319 | E |
| I-321 | D |
| I-322 | D |
| I-323 | B |

### Example 3. Phosphorylated STAT6 (pSTAT6) Flow Cytometry Assay in Human A549 Cells

Phosphorylation of STAT6 in human A549 cells was quantitatively measured using flow cytometry technology.

The STAT6 L4 pSTAT6 results are shown in Table 23. The letter codes for IC₅₀ (μM) include: A (<0.1 μM); B (0.1-1 μM); C (>1-10 μM); D (>10-100 μM); and E (>100 μM or not tested).

TABLE 23
STAT6 IL4 pSTAT6 Results.

| | STAT6 IL4 |
|---|---|
| | pSTAT6 assay |
| | in A549 cells: |
| | Average IC₅₀ |
| I-# | (μM) |
| I-324 | A |
| I-325 | A |
| I-326 | D |
| I-327 | A |
| I-328 | A |
| I-329 | A |
| I-330 | A |
| I-331 | A |
| I-332 | A |
| I-333 | A |
| I-334 | A |
| I-335 | A |
| I-336 | A |
| I-337 | A |
| I-338 | A |
| I-339 | A |
| I-340 | A |
| I 341 | A |
| I-342 | A |
| I-343 | A |
| I-344 | A |
| I-345 | A |
| I-346 | A |
| I-347 | A |
| I-348 | A |
| I-349 | A |
| I-350 | A |
| I-351 | A |
| I-352 | A |
| I-353 | A |
| I-354 | A |
| I-355 | A |
| I-356 | A |
| I-357 | A |
| I-358 | A |
| I-359 | A |
| I-360 | A |
| I-361 | A |
| I-362 | A |
| I-363 | A |
| I-364 | A |
| I-365 | A |
| I-366 | A |
| I-367 | A |
| I_368 | A |
| I-369 | A |
| I-370 | A |
| I-371 | A |
| I-372 | B |
| I-373 | B |
| I-374 | A |
| I-375 | B |
| I-376 | E |
| I-377 | E |
| I-378 | E |
| I-379 | A |
| I-380 | E |
| I-381 | E |
| I-382 | E |
| I-383 | B |
| I-384 | A |
| I-385 | A |
| I-386 | A |
| I-387 | A |
| I-388 | A |
| I-389 | A |
| I-390 | A |
| I-391 | A |
| I-392 | A |
| I-393 | A |
| I-394 | E |
| I-395 | E |
| I-396 | E |
| I-397 | E |
| I-398 | E |
| I-399 | E |
| I-400 | E |
| I-401 | E |
| I-402 | E |
| I-403 | E |
| I-404 | E |
| I-405 | E |
| I-406 | E |
| I-407 | E |
| I-408 | E |
| I-409 | E |
| I-410 | E |
| I-411 | E |
| I-412 | E |
| I-413 | B |
| I-414 | B |
| I-415 | A |
| I-416 | A |
| I-417 | A |
| I-418 | A |
| I-419 | A |
| I-420 | B |
| I-421 | A |
| I-422 | A |
| I-423 | A |
| I-424 | A |
| I-425 | A |
| I-426 | A |
| I-427 | A |
| I-428 | A |
| I-429 | A |
| I-430 | B |
| I-431 | A |
| I-432 | A |
| I-433 | E |
| I-434 | A |
| I-435 | A |
| I-436 | A |
| I-437 | A |
| I-438 | A |
| I-439 | A |
| I-440 | A |
| I-441 | A |
| I-442 | A |
| I-443 | A |
| I-444 | A |
| I-445 | A |
| I-446 | C |
| I-447 | A |
| I-448 | A |
| I-449 | A |
| I-450 | A |
| I-451 | A |
| I-452 | E |
| I-453 | A |
| I-454 | A |
| I-455 | A |
| I-456 | A |
| I-457 | A |
| I-458 | A |
| I-459 | A |
| I-460 | A |
| I-461 | B |
| I-462 | B |
| I-463 | A |
| I-464 | A |
| I-465 | E |
| I-466 | A |
| I-467 | A |
| I-468 | A |
| I-469 | B |
| I-470 | A |
| I-471 | A |
| I-472 | A |
| I-473 | A |
| I-474 | A |
| I-475 | A |
| I-476 | A |
| I-477 | A |
| I-478 | A |
| I-479 | A |
| I-480 | A |
| I-481 | A |
| I-482 | A |
| I-483 | A |
| I-484 | E |
| I-485 | A |
| I-486 | A |
| I-487 | A |
| I-488 | A |
| I-489 | A |
| I-490 | E |
| I-491 | E |
| I-492 | E |
| I-493 | E |
| I-494 | E |
| I-495 | E |
| I-496 | E |
| I-497 | D |
| I-498 | D |
| I-499 | D |
| I-500 | E |
| I-501 | D |
| I-502 | D |
| I-503 | D |
| I-504 | D |
| I-505 | D |
| I-506 | D |
| I-507 | D |
| I-508 | A |
| I-509 | A |
| I-510 | A |
| I-511 | A |
| I-512 | A |
| I-513 | A |
| I-514 | A |
| I-515 | A |
| I-516 | A |
| I-517 | A |
| I-518 | A |
| I-519 | A |
| I-520 | A |
| I-521 | A |
| I-522 | A |
| I-523 | A |
| I-524 | A |
| I-525 | A |
| I-526 | A |
| I-527 | A |
| I-528 | B |
| I-529 | A |
| I-530 | A |
| I-531 | A |
| I-532 | A |
| I-533 | A |
| I-534 | E |
| I-535 | E |
| I-536 | A |
| I-537 | A |
| I-538 | A |
| I-539 | A |
| I-540 | A |
| I-541 | A |
| I-542 | E |
| I-543 | A |
| I-544 | A |
| I-545 | A |
| I-546 | E |
| I-547 | E |
| I-548 | E |
| I-549 | E |
| I-550 | E |
| I-551 | A |
| I-552 | B |
| I-553 | B |
| I-554 | B |
| I-555 | B |
| I-556 | A |
| I-557 | A |
| I-558 | A |
| I-559 | A |
| I-560 | A |
| I-561 | A |
| I-562 | A |
| I-563 | A |
| I-564 | A |
| I-565 | A |
| I-566 | A |
| I-567 | A |
| I-568 | A |
| I-569 | A |
| I-570 | A |
| I-571 | A |
| I-572 | A |
| I-573 | A |
| I-574 | A |
| I-575 | A |
| I-576 | A |
| I-577 | A |
| I-578 | A |
| I-579 | A |
| I-580 | A |
| I-581 | A |
| I-582 | A |
| I-583 | E |
| I-584 | A |
| I-585 | A |
| I-586 | A |
| I-587 | A |
| I-588 | A |
| I-589 | A |
| I-590 | A |
| I-591 | A |
| I-592 | A |
| I-593 | A |
| I-594 | A |
| I-595 | A |
| I-596 | A |
| I-597 | A |
| I-598 | A |
| I-599 | A |
| I-600 | A |
| I-601 | A |
| I-602 | A |
| I-603 | A |
| I-604 | A |
| I-605 | A |
| I-606 | A |
| I-607 | E |
| I-608 | E |
| I-609 | A |
| I-610 | A |
| I-611 | A |
| I-612 | A |
| I-613 | A |
| I-614 | A |
| I-615 | A |
| I-616 | E |
| I-617 | A |
| I-618 | A |
| I-619 | B |
| I-620 | A |
| I-621 | A |
| I-622 | A |
| I-623 | A |
| I-624 | A |
| I-625 | A |
| I-626 | A |
| I-627 | A |
| I-628 | A |
| I-629 | A |
| I-630 | A |
| I-631 | A |
| I-632 | A |
| I-633 | A |
| I-634 | A |
| I-635 | A |
| I-636 | A |
| I-637 | A |
| I-638 | A |
| I-639 | A |
| I-640 | A |
| I-641 | A |
| I-642 | B |
| I-643 | A |
| I-644 | A |
| I-645 | A |
| I-646 | A |
| I-647 | A |
| I-648 | A |
| I-649 | A |
| I-650 | A |
| I-651 | A |
| I-652 | A |
| I-653 | A |
| I-654 | A |
| I-655 | A |
| I-656 | A |
| I-657 | A |
| I-658 | A |
| I-659 | A |
| I-660 | A |
| I-661 | A |
| I-662 | A |
| I-663 | A |
| I-664 | A |
| I-665 | A |
| I-666 | E |
| I-667 | E |
| I-668 | E |
| I-669 | A |
| I-670 | A |
| I-671 | B |
| I-672 | A |
| I-673 | A |
| I-674 | B |
| I-675 | A |
| I-676 | B |
| I-677 | A |
| I-678 | A |
| I-679 | A |
| I-680 | A |
| I-681 | A |
| I-682 | A |
| I-683 | A |
| I-684 | A |
| I-685 | A |
| I-686 | A |
| I-687 | A |
| I-688 | A |
| I-689 | A |
| I-690 | A |
| I-691 | A |
| I-692 | E |
| I-693 | E |
| I-694 | E |
| I-695 | E |
| I-696 | E |
| I-697 | E |
| I-698 | D |
| I-699 | D |
| I-700 | D |
| I-701 | D |
| I-702 | D |
| I-703 | D |
| I-704 | D |
| I-705 | D |
| I-706 | D |
| I-707 | D |
| I-708 | D |
| I-709 | D |
| I-710 | D |
| I-711 | D |
| I-712 | D |
| I-713 | D |
| I-714 | D |
| I-715 | D |
| I-716 | D |
| I-717 | D |
| I-718 | D |
| I-719 | D |
| I-720 | D |
| I-721 | D |
| I-722 | D |
| I-723 | D |
| I-724 | A |
| I-725 | A |
| I-726 | A |
| I-727 | A |
| I-728 | E |
| I-729 | A |
| I-730 | A |
| I-731 | A |
| I-732 | E |
| I-733 | A |
| I-734 | A |
| I-735 | A |
| I-736 | A |
| I-737 | A |
| I-738 | A |
| I-739 | A |
| I-740 | A |
| I-741 | B |
| I-742 | A |
| I-743 | A |
| I-744 | A |
| I-745 | A |
| I-746 | A |
| I-747 | A |
| I-748 | A |
| I-749 | A |
| I-750 | E |
| I-751 | A |
| I-752 | A |
| I-753 | A |
| I-754 | A |
| I-755 | A |
| I-756 | A |
| I-757 | A |
| I-758 | A |
| I-759 | A |
| I-760 | E |
| I-761 | E |
| I-762 | E |
| I-763 | A |
| I-764 | A |
| I-765 | A |
| I-766 | E |
| I-767 | E |
| I-768 | E |
| I-769 | A |
| I-770 | C |
| I-771 | A |
| I-772 | E |
| I-773 | A |
| I-774 | A |
| I-775 | A |
| I-776 | A |
| I-777 | A |
| I-778 | A |
| I-779 | A |
| I-780 | A |
| I-781 | A |
| I-782 | A |
| I-783 | A |
| I-784 | A |
| I-785 | A |
| I-786 | A |
| I-787 | A |
| I-788 | A |
| I-789 | A |
| I-790 | A |
| I-791 | A |
| I-792 | A |
| I-793 | A |
| I-794 | A |
| I-795 | E |
| I-796 | E |
| I-797 | E |
| I-798 | E |
| I-799 | E |
| I-800 | E |
| I-801 | E |
| I-802 | B |
| I-803 | D |
| I-804 | B |
| I-805 | D |
| I-806 | D |
| I-807 | A |
| I-808 | B |
| I-809 | E |
| I-810 | E |
| I-811 | E |
| I-812 | E |
| I-813 | E |
| I-814 | E |
| I-815 | B |
| I-816 | B |
| I-817 | B |
| I-818 | B |
| I-819 | B |
| I-820 | B |
| I-821 | B |
| I-822 | B |
| I-823 | A |
| I-824 | B |
| I-825 | B |
| I-826 | B |
| I-827 | B |
| I-828 | B |
| I-829 | B |
| I-830 | B |
| I-831 | B |
| I-832 | C |
| I-833 | B |
| I-834 | B |
| I-835 | B |
| I-837 | B |
| I-838 | A |
| I-839 | A |
| I-840 | A |
| I-841 | A |
| I-842 | A |
| I-843 | A |
| I-844 | B |
| I-845 | A |
| I-846 | A |
| I-847 | B |
| I-848 | A |
| I-849 | A |
| I-850 | A |
| I-851 | A |
| I-852 | B |
| I-853 | A |
| I-854 | A |
| I-855 | A |
| I-856 | A |
| I-857 | B |
| I-858 | A |
| I-859 | A |
| I-860 | B |
| I-861 | A |
| I-862 | A |
| I-863 | A |
| I-864 | A |
| I-865 | A |
| I-866 | A |
| I-867 | A |
| I-868 | B |
| I-869 | A |
| I-870 | A |
| I-871 | A |
| I-872 | B |
| I-873 | A |
| I-874 | B |
| I-875 | A |
| I-876 | B |
| I-877 | B |
| I-878 | A |
| I-879 | A |
| I-880 | B |
| I-881 | B |
| I-882 | B |
| I-883 | B |
| I-884 | B |
| I-885 | B |
| I-886 | B |
| I-887 | B |
| I-888 | B |
| I-889 | A |
| I-890 | B |
| I-891 | B |
| I-892 | A |
| I-893 | A |
| I-894 | B |
| I-895 | A |
| I-896 | B |
| I-897 | B |
| I-898 | A |
| I-899 | A |
| I-900 | A |
| I-901 | A |
| I-902 | C |
| I-903 | B |
| I-904 | B |
| I-905 | A |
| I-906 | A |
| I-907 | A |
| I-908 | A |
| I-909 | A |
| I-910 | A |
| I-911 | B |
| I-912 | B |
| I-913 | B |
| I-914 | A |
| I-915 | A |
| I-916 | B |
| I-917 | A |
| I-918 | A |
| I-919 | A |
| I-920 | A |
| I-921 | B |
| I-922 | B |
| I-923 | A |
| I-924 | A |
| I-925 | B |
| I-926 | A |
| I-927 | B |
| I-928 | A |
| I-929 | A |
| I-930 | B |
| I-931 | B |
| I-932 | B |
| I-933 | A |
| I-934 | A |
| I-935 | A |
| I-936 | A |
| I-937 | B |
| I-938 | A |
| I-939 | A |
| I-940 | A |
| I-941 | A |
| I-942 | A |
| I-943 | A |
| I-944 | A |
| I-945 | B |
| I-946 | A |
| I-947 | C |
| I-948 | A |
| I-949 | A |
| I-950 | A |
| I-951 | A |
| I-952 | A |
| I-953 | A |
| I-954 | A |
| I-955 | A |
| I-956 | C |
| I-957 | B |
| I-958 | B |
| I-959 | A |
| I-960 | A |
| I-961 | A |
| I-962 | A |
| I-963 | A |
| I-964 | B |
| I-965 | B |
| I-966 | A |
| I-967 | B |
| I-968 | B |
| I-969 | B |
| I-970 | A |
| I-971 | A |
| I-972 | A |
| I-973 | B |
| I-974 | A |
| I-975 | A |
| I-976 | A |
| I-977 | A |
| I-978 | B |
| I-979 | B |
| I-980 | A |
| I-981 | B |
| I-982 | E |
| I-983 | A |
| I-984 | A |
| I-985 | A |
| I-986 | A |
| I-987 | C |
| I-988 | A |
| I-989 | A |
| I-990 | A |
| I-991 | A |
| I-992 | A |
| I-993 | B |
| I-994 | A |
| I-995 | A |
| I-996 | A |
| I-997 | A |
| I-998 | A |
| I-999 | A |
| I-1000 | A |
| I-1001 | A |
| I-1002 | A |
| I-1003 | A |
| I-1004 | A |
| I-1005 | A |
| I-1006 | B |
| I-1007 | A |
| I-1008 | A |
| I-1009 | A |
| I-1010 | A |
| I-1011 | A |
| I-1012 | A |
| I-1013 | A |
| I-1014 | A |
| I-1015 | A |
| I-1016 | A |
| I-1017 | A |
| I-1018 | A |
| I-1019 | A |
| I-1020 | A |
| I-1021 | A |
| I-1022 | A |
| I-1023 | A |
| I-1024 | A |
| I-1025 | A |
| I-1026 | A |
| I-1027 | B |
| I-1028 | C |
| I-1029 | A |
| I-1030 | A |
| I-1031 | A |
| I-1032 | B |
| I-1033 | A |
| I-1034 | A |
| I-1035 | B |
| I-1036 | A |
| I-1037 | B |
| I-1038 | B |
| I-1039 | A |
| I-1040 | A |
| I-1041 | A |
| I-1042 | D |
| I-1043 | E |
| I-1044 | C |
| I-1045 | B |
| I-1046 | B |
| I-1047 | B |
| I-1048 | A |
| I-1049 | B |
| I-1050 | A |
| I-1051 | E |
| I-1052 | A |
| I-1053 | A |
| I-1054 | A |
| I-1055 | A |
| I-1056 | A |
| I-1057 | A |
| I-1058 | A |
| I-1059 | A |
| I-1060 | A |
| I-1061 | A |
| I-1062 | A |
| I-1063 | A |
| I-1064 | A |
| I-1065 | A |
| I-1066 | A |
| I-1067 | A |
| I-1068 | A |
| I-1069 | A |
| I-1070 | A |
| I-1071 | A |
| I-1072 | A |
| I-1073 | A |
| I-1074 | A |
| I-1075 | A |
| I-1076 | A |
| I-1077 | A |
| I-1078 | A |
| I-1079 | A |
| I-1080 | A |
| I-1081 | A |
| I-1082 | A |
| I-1083 | A |
| I-1084 | A |
| I-1085 | A |
| I-1086 | A |
| I-1087 | A |
| I-1088 | A |
| I-1089 | A |
| I-1090 | A |
| I-1091 | B |
| I-1092 | A |
| I-1093 | B |
| I-1094 | A |
| I-1095 | A |
| I-1096 | A |
| I-1097 | E |
| I-1098 | A |
| I-1099 | B |
| I-1100 | A |
| I-1101 | A |
| I-1103 | A |
| I-1104 | B |
| I-1105 | B |
| I-1106 | C |
| I-1107 | B |
| I-1108 | A |
| I-1109 | A |
| I-1110 | A |
| I-1111 | A |
| I-1112 | A |
| I-1113 | B |
| I-1114 | A |
| I-1115 | A |
| I-1116 | A |
| I-1117 | A |
| I-1118 | A |
| I-1119 | A |
| I-1120 | A |
| I-1121 | A |
| I-1122 | A |
| I-1123 | A |
| I-1124 | B |
| I-1125 | A |
| I-1126 | A |
| I-1127 | B |
| I-1128 | A |
| I-1129 | A |
| I-1130 | B |
| I-1131 | A |
| I-1132 | A |
| I-1133 | A |
| I-1134 | A |
| I-1135 | A |
| I-1136 | A |
| I-1137 | A |
| I-1138 | A |
| I-1139 | A |
| I-1140 | A |
| I-1141 | A |
| I-1142 | A |
| I-1143 | A |
| I-1144 | A |
| I-1145 | A |
| I-1146 | A |
| I-1147 | A |
| I-1148 | A |
| I-1149 | A |
| I-1150 | A |
| I-1151 | A |
| I-1152 | A |
| I-1153 | A |
| I-1154 | A |
| I-1155 | A |
| I-1156 | A |
| I-1157 | A |
| I-1158 | A |
| I-1159 | B |
| I-1160 | A |
| I-1161 | B |
| I-1162 | C |
| I-1163 | B |
| I-1164 | B |
| I-1165 | A |
| I-1166 | A |
| I-1167 | A |
| I-1168 | B |
| I-1169 | B |
| I-1170 | B |
| I-1171 | B |
| I-1172 | B |
| I-1173 | B |
| I-1174 | C |
| I-1175 | B |
| I-1176 | A |
| I-1177 | B |
| I-1178 | B |
| I-1179 | A |
| I-1180 | A |
| I-1181 | A |
| I-1182 | A |
| I-1183 | A |
| I-1184 | A |
| I-1185 | A |
| I-1186 | A |
| I-1187 | A |
| I-1188 | B |
| I-1189 | B |
| I-1190 | B |
| I-1191 | C |
| I-1192 | C |
| I-1193 | A |
| I-1194 | A |
| I-1195 | E |
| I-1196 | A |
| I-1197 | B |
| I-1198 | A |
| I-1199 | B |
| I-1200 | A |
| I-1201 | A |
| I-1202 | A |
| I-1203 | C |
| I-1204 | C |
| I-1205 | C |

LENGTHY TABLES
The patent application contains a lengthy table section. A copy of the table is available in electronic form from the USPTO web site (https://seqdata.uspto.gov/?pageRequest=docDetail&DocID=US20250214977A1). An electronic copy of the table will also be available from the USPTO upon request and payment of the fee set forth in 37 CFR 1.19(b)(3).

1. An inhibitor compound of formula I′: 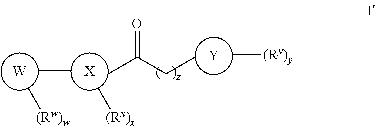
or a pharmaceutically acceptable salt thereof, wherein:
Ring W and its R^{w} substituents is 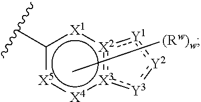
each of X¹, X⁴, and X⁵ is independently CH, CR^{w}, or N;
each of X² and X³ is independently C or N,
wherein at most one of X¹, X², X³, X⁴, and X⁵ is N;
each of Y¹, Y², and Y³ is independently CH, CR^{w}, CH₂, CH(R^{w}), C(R^{w})₂, NH, NR^{w}, N, O, or S,
wherein at least one of Y² and Y³ is CH, CR^{w}, CH₂, CH(R^{w}), or C(R^{w})₂;
each [symbol] represents a single bond or a double bond;
Ring X and its R^{x} substituents is 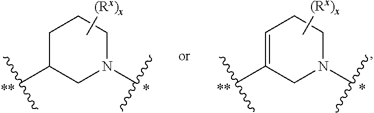
wherein 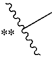
represents a bond to Ring W, and 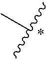
represents a bond to the rest of the molecule;
Ring Y is a ring selected from phenyl, 3-8 membered saturated or partially unsaturated monocyclic carbocyclyl or heterocyclyl with 1-3 heteroatoms independently selected from nitrogen, oxygen, and sulfur, and 5-6 membered monocyclic heteroaryl with 1-4 heteroatoms independently selected from nitrogen, oxygen and sulfur;
R^{w} is selected from hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R^{B} is independently -L^{B}-Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, a 5-10 membered monocyclic or bicyclic arylenyl or heteroarylenyl having 1-4 heteroatoms independently selected from nitrogen, oxygen, and sulfur; or a 5-15 membered saturated or partially saturated monocyclic, bicyclic, or tricyclic carbocyclylenyl, heterocyclylenyl, arylenyl, or heteroarylenyl having 1-4 heteroatoms independently selected from nitrogen, oxygen, and sulfur;
each Cy^{B2} is independently an optionally substituted ring selected from phenyl, a 3-10 membered saturated or partially unsaturated monocyclic or bicyclic carbocyclic or heterocyclic ring having 1-2 heteroatoms independently selected from nitrogen, oxygen, and sulfur, a 5-10 membered monocyclic or bicyclic aryl or heteroaryl with 1-4 heteroatoms independently selected from nitrogen, oxygen and sulfur; or a 5-15 membered saturated or partially saturated monocyclic, bicyclic, or tricyclic carbocyclyl, heterocyclyl, aryl, or heteroaryl having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom or adjacent atoms are optionally taken together with their intervening atoms to form a 3-7 membered saturated or partially unsaturated ring having 0-3 heteroatoms, in addition to the atom or adjacent atoms to which they are attached, independently selected from nitrogen, oxygen, and sulfur; and
each of w, x, y, and z are independently 0, 1, 2, 3, or 4, and wherein:
(i) when Ring X and its R^{x} substituents is 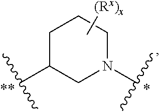
X¹ is N, and Y¹ is NH, NR^{w}, or N, then Y² and Y³ are not NH, NR^{w}, or N;
(ii) when Ring X and its R^{x} substituents is 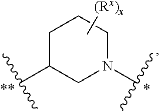
each of X¹, X⁴, and X⁵ is CH or CR^{w}, each of X² and X³ is C, and each of Y² and Y³ is CH, CR^{w}, CH₂, CH(R^{w}), or C(R^{w})₂, then Y¹ is not CH, CR^{w}, CH₂, CH(R^{w}), or C(R^{w})₂;
(iii) when Ring X and its R^{x} substituents is 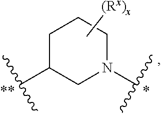
is N, and Y¹ is S, then Ring Y is not phenyl or 3-8 membered saturated or partially unsaturated monocyclic carbocyclyl or heterocyclyl with 1-3 heteroatoms independently selected from nitrogen, oxygen, and sulfur;
(iv) when Ring X and its R^{x} substituents is 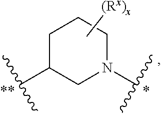
X¹ is N, and Y¹ is NH, NR^{w}, or N, then Y³ is not S;
(v) when Ring X and its R^{x} substituents is 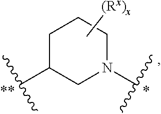
X¹ is N, and Y¹ is O, then Y² is not NH, NR^{w}, or N;
(vi) when Ring X and its R^{x} substituents is 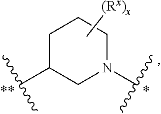
X¹ is N, Y¹ is NH, NR^{w}, or N, and Y² and Y³ are CH, CR^{w}, CH₂, CH(R^{w}), or C(R^{w})₂, then z is not 0 or 1;
(vii) when Ring X and its R^{x} substituents is 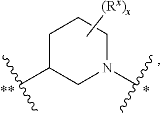
each of X¹, X⁴, and X⁵ is CH or CR^{w}, each of X² and X³ is C, and Y² is CH, CR^{w}, CH₂, CH(R^{w}), or C(R^{w})₂, then only one of Y² and Y³ is O;
(viii) when Ring X and its R^{x} substituents is 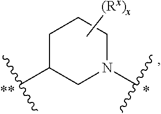
each of X¹, X⁴, and X⁵ is CH or CR^{w}, each of X² and X³ is C, Y¹ is O, NH, NR^{w}, or N and Y² and Y³ are CH, CR^{w}, CH₂, CH(R^{w}), or C(R^{w})₂, then z is not 0 or 1; and
(ix) when Ring X and its R^{x} substituents is 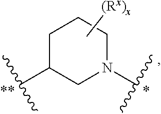
X⁵ is N, and Y¹ is NH, NR^{w}, or N, then Y² is not NH, NR^{w}, or N, and
wherein the inhibitor compound is not of structure A-B-C defined by the combination of the building blocks A, B, and C within the table below:

| A | B | C |
|---|---|---|
| 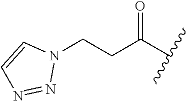 [SMILES: *C(=O)CCn1ccnn1] | 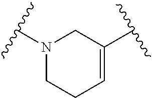 [SMILES: *C1=CCCN(*)C1] | 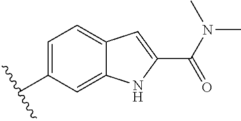 [SMILES: *c1ccc2cc(C(=O)N(C)C)[nH]c2c1] |
| 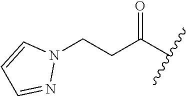 [SMILES: *C(=O)CCn1cccn1] | | 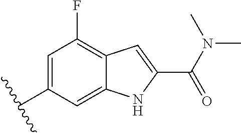 [SMILES: *c1cc(F)c2cc(C(=O)N(C)C)[nH]c2c1] |
| 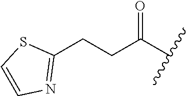 [SMILES: *C(=O)CCc1nccs1] | | 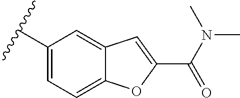 [SMILES: *c1ccc2oc(C(=O)N(C)C)cc2c1] |
| 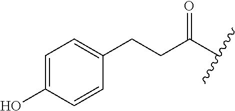 [SMILES: *C(=O)CCc1ccc(O)cc1] | | 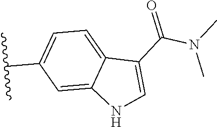 [SMILES: *c1ccc2c(C(=O)N(C)C)c[nH]c2c1] |
| 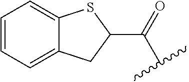 [SMILES: *C(=O)C1Cc2ccccc2S1] | | |

2-4. (canceled)

5. The inhibitor compound of claim 1, wherein Ring Y is

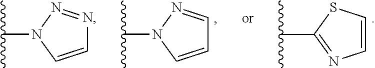

6-8. (canceled)

9. The inhibitor compound of claim 1, wherein the inhibitor compound is of formula I-a-1b or I-a-2b: 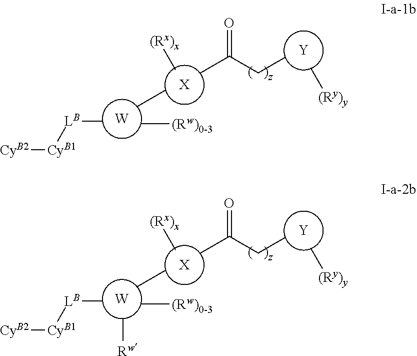
or a pharmaceutically acceptable salt thereof, wherein:
R^{w′} is hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, or —NRS(O)₂R.

10. (canceled)

11. The inhibitor compound of claim 1, wherein the inhibitor compound is of formula I-b-5, I-b-6, I-c-5, I-c-6, I-d-5, I-d-6, I-e-5, or I-e-6: 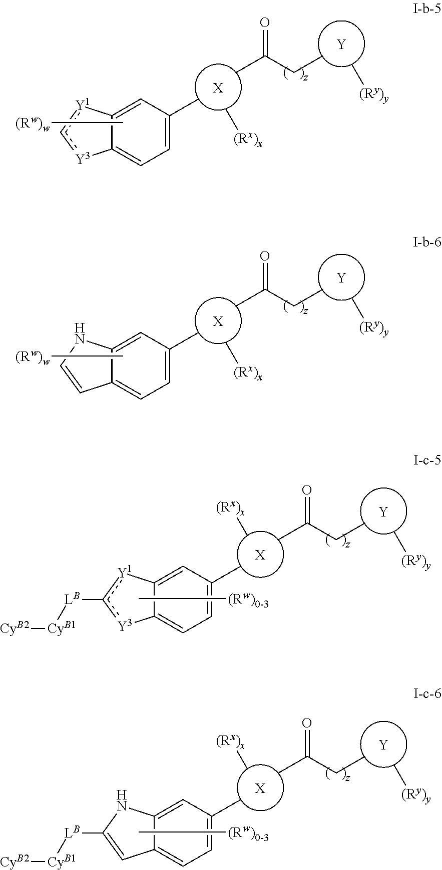 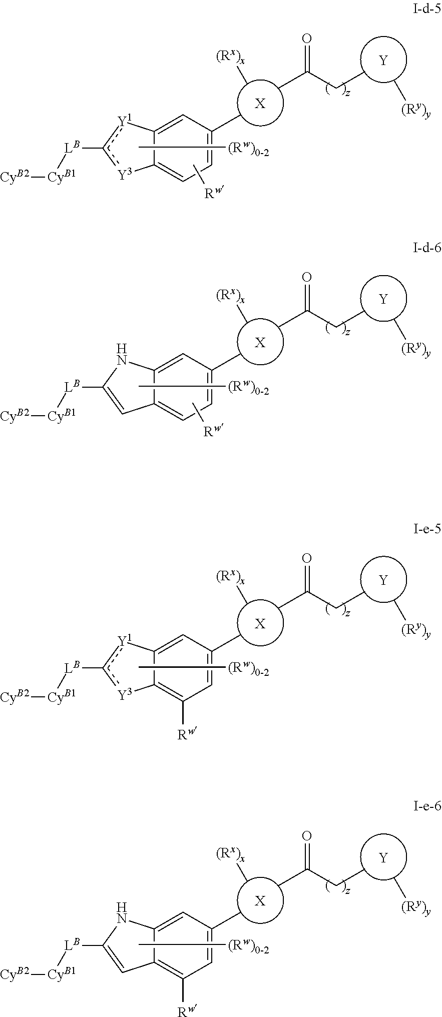
or a pharmaceutically acceptable salt thereof, wherein:
R^{w′} is hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, or —NRS(O)₂R.

12-17. (canceled)

18. The inhibitor compound of claim 1, wherein:
(i) Ring W and its R^{w} substituents is 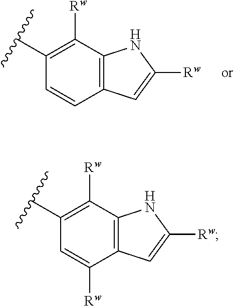
Ring Y is a 5-6 membered monocyclic heteroaryl with 1-4 heteroatoms independently selected from nitrogen, oxygen and sulfur;
z is 2;
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-7 membered saturated or partially unsaturated carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur; and
each Cy^{B2} is independently an optionally substituted ring selected from phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur; or
(ii) Ring W and its R^{w} substituents is 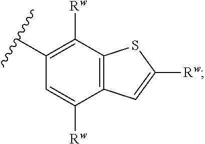
Ring Y is a 5-6 membered monocyclic heteroaryl with 1-4 heteroatoms independently selected from nitrogen, oxygen and sulfur;
z is 2;
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-7 membered saturated or partially unsaturated carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur; and
each Cy^{B2} is independently an optionally substituted ring selected from phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur; or
(iii) Ring W and its R^{w} substituents is 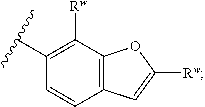
Ring Y is a 5-6 membered monocyclic heteroaryl with 1-4 heteroatoms independently selected from nitrogen, oxygen and sulfur;
z is 2;
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-7 membered saturated or partially unsaturated carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur; and
each Cy^{B2} is independently an optionally substituted ring selected from phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur.

19. (canceled)

20. The inhibitor compound of claim 1, wherein the inhibitor compound is selected from Table 1B, or a pharmaceutically acceptable salt thereof.

21. A compound selected from Table 2B, or a pharmaceutically acceptable salt thereof.

22. A pharmaceutical composition comprising a inhibitor compound of claim 1, or a pharmaceutically acceptable salt thereof, and a pharmaceutically acceptable carrier, adjuvant, or vehicle.

23. A method of inhibiting STAT6, the method comprising contacting STAT6 with an inhibitor compound of claim 1, or a pharmaceutically acceptable salt thereof, or a pharmaceutical composition thereof.

24. The method of claim 23, wherein the contacting occurs in a patient or a biological sample.

25. A method of inhibiting STAT6, the method comprising contacting STAT6 with an inhibitor compound of formula I: 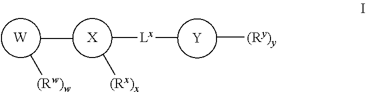
or a pharmaceutically acceptable salt thereof, wherein:
Ring W and its R^{w} substituents is 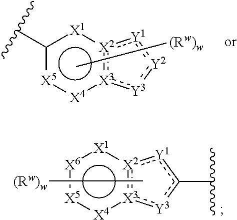
each of X¹, X⁴, X⁵ and X⁶ is independently CH, CR^{w}, or N;
each of X² and X³ is independently C or N,
wherein at most one of X¹, X², X³, X⁴, X⁵ and X⁶ is N;
each of Y¹, Y², and Y³ is independently CH, CR^{w}, CH₂, CH(R^{w}), C(R^{w})₂, NH, NR^{w}, N, O, or S,
wherein at least one of Y² and Y³ is CH, CR^{w}, CH₂, CH(R^{w}), or C(R^{w})₂;
each [symbol] represents a single bond or a double bond;
Ring X and its R^{x} substituents is 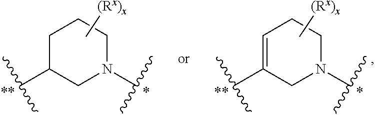
wherein 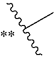
represents a bond to Ring W, and 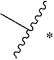
represents a bond to L^{x};
Ring Y is a ring selected from phenyl, naphthyl, 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-10 membered monocyclic or bicyclic heteroaryl with 1-4 heteroatoms independently selected from nitrogen, oxygen and sulfur;
L^{x} is 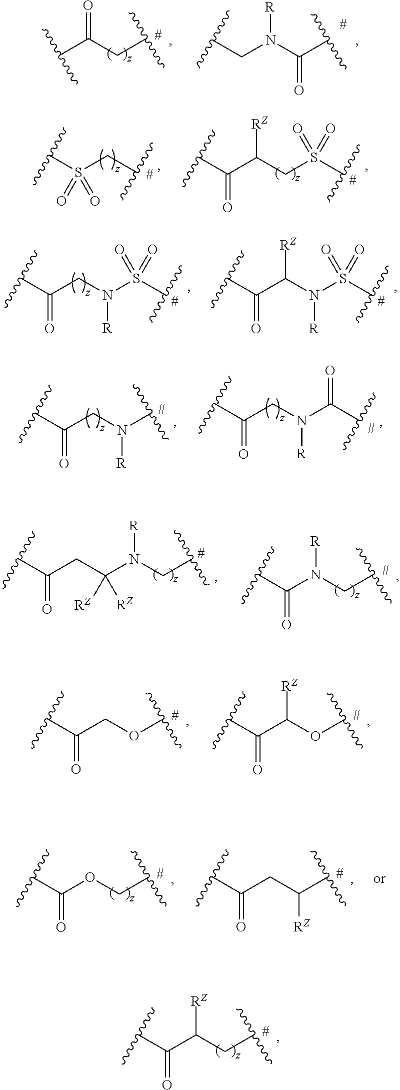
wherein 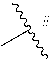
represents a bond to Ring Y;
R^{w} is selected from hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
R^{x} and R^{y} are independently selected from hydrogen, R^{A}, halogen, —CN, —NO₂, —OR, —SR, NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
R^{Z} is R^{A}, —OR, or NR₂;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R^{B} is independently -L^{B}-Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂—, —S(O)(NR)—, or —CR═CR—;
each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, a 5-10 membered monocyclic or bicyclic arylenyl or heteroarylenyl having 1-4 heteroatoms independently selected from nitrogen, oxygen, and sulfur; or a 5-15 membered saturated or partially saturated monocyclic, bicyclic, or tricyclic carbocyclylenyl, heterocyclylenyl, arylenyl, or heteroarylenyl having 1-4 heteroatoms independently selected from nitrogen, oxygen, and sulfur;
each Cy^{B2} is independently an optionally substituted ring selected from phenyl, a 3-10 membered saturated or partially unsaturated monocyclic or bicyclic carbocyclic or heterocyclic ring having 1-2 heteroatoms independently selected from nitrogen, oxygen, and sulfur, a 5-10 membered monocyclic or bicyclic aryl or heteroaryl with 1-4 heteroatoms independently selected from nitrogen, oxygen and sulfur; or a 5-15 membered saturated or partially saturated monocyclic, bicyclic, or tricyclic carbocyclyl, heterocyclyl, aryl, or heteroaryl having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom or adjacent atoms are optionally taken together with their intervening atoms to form a 3-7 membered saturated or partially unsaturated ring having 0-3 heteroatoms, in addition to the atom or adjacent atoms to which they are attached, independently selected from nitrogen, oxygen, and sulfur; and
each of w, x, y, and z are independently 0, 1, 2, 3, or 4.

26. The method of claim 25, wherein the inhibitor compound is of formula I′: 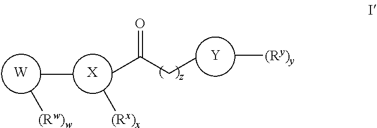
or a pharmaceutically acceptable salt thereof, wherein:
Ring W and its R^{w} substituents is 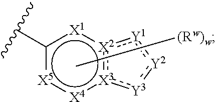
and
Ring Y is a ring selected from phenyl, 3-8 membered saturated or partially unsaturated monocyclic carbocyclyl or heterocyclyl with 1-3 heteroatoms independently selected from nitrogen, oxygen, and sulfur, and 5-6 membered monocyclic heteroaryl with 1-4 heteroatoms independently selected from nitrogen, oxygen and sulfur.

27. The method of claim 25, wherein the inhibitor compound is of formula II: 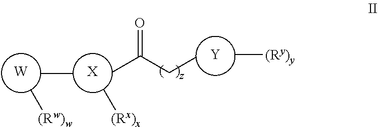
or a pharmaceutically acceptable salt thereof, wherein:
Ring W and its R^{w} substituents is 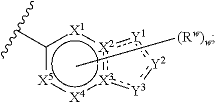
and
Ring Y is a ring selected from naphthyl, 5-10 membered saturated or partially unsaturated bicyclic or bridged bicyclic carbocyclyl or heterocyclyl with 1-3 heteroatoms independently selected from nitrogen, oxygen, and sulfur, and 8-10 membered bicyclic heteroaryl with 1-4 heteroatoms independently selected from nitrogen, oxygen and sulfur.

28. The method of claim 25, wherein the inhibitor compound is of formula III: 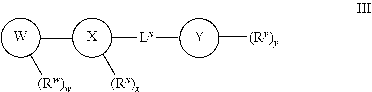
or a pharmaceutically acceptable salt thereof, wherein:
Ring W and its R^{w} substituents is 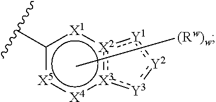
Ring Y is a ring selected from phenyl, 3-8 membered saturated or partially unsaturated monocyclic carbocyclyl or heterocyclyl with 1-3 heteroatoms independently selected from nitrogen, oxygen, and sulfur, 5-6 membered monocyclic heteroaryl with 1-4 heteroatoms independently selected from nitrogen, oxygen and sulfur, naphthyl, 5-10 membered saturated or partially unsaturated bicyclic or bridged bicyclic carbocyclyl or heterocyclyl with 1-3 heteroatoms independently selected from nitrogen, oxygen, and sulfur, and 8-10 membered bicyclic heteroaryl with 1-4 heteroatoms independently selected from nitrogen, oxygen and sulfur; and
L^{x} is 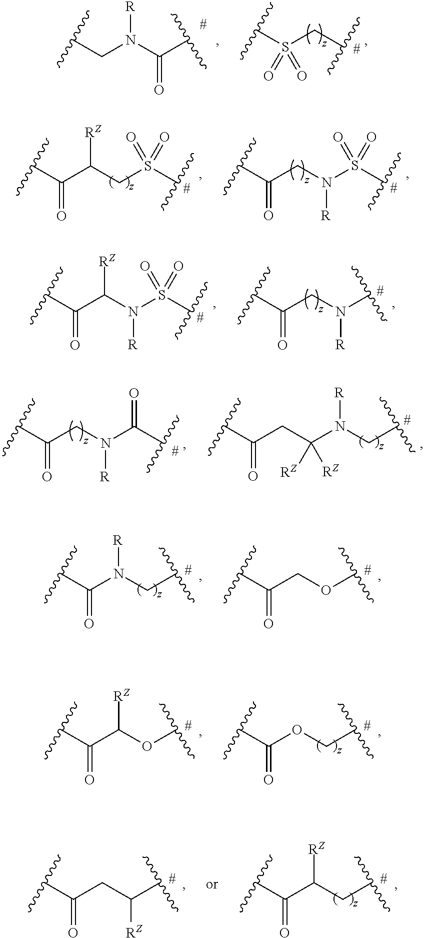
wherein 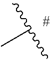
represents a bond to Ring Y.

29-30. (canceled)

31. The method of claim 25, wherein the inhibitor compound is of formula IV: 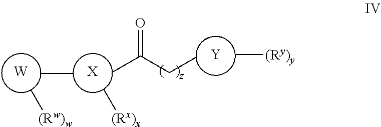
or a pharmaceutically acceptable salt thereof, wherein:
Ring W and its R^{w} substituents is 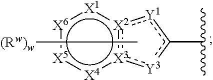
and
Ring Y is a ring selected from phenyl, 3-8 membered saturated or partially unsaturated monocyclic carbocyclyl or heterocyclyl with 1-3 heteroatoms independently selected from nitrogen, oxygen, and sulfur, and 5-6 membered monocyclic heteroaryl with 1-4 heteroatoms independently selected from nitrogen, oxygen and sulfur.

32-38. (canceled)

39. The method of claim 25, wherein the inhibitor compound is of formula I-a-1b, I-a-2a, I-a-1b, or I-a-2b: 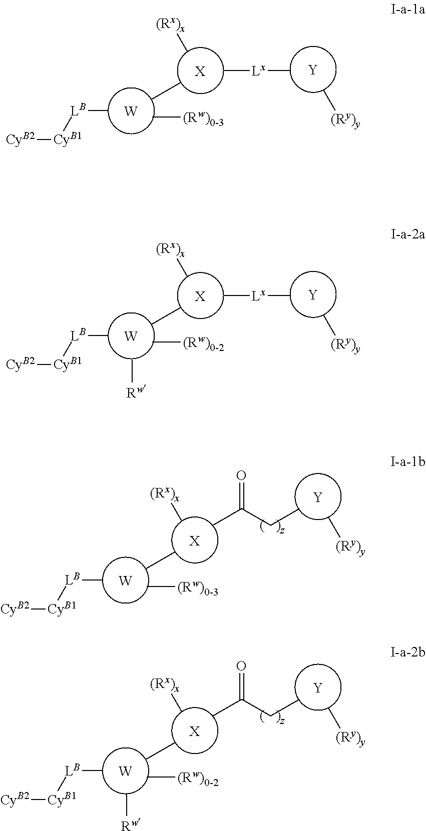
or a pharmaceutically acceptable salt thereof, wherein:
R^{w′} is hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

40. (canceled)

41. The method of claim 25, wherein the inhibitor compound is of formula I-b-1 through I-b-6, I-c-1 through I-c-6, formula I-d-1 through I-d-6, or formula I-e-1 through I-e-6 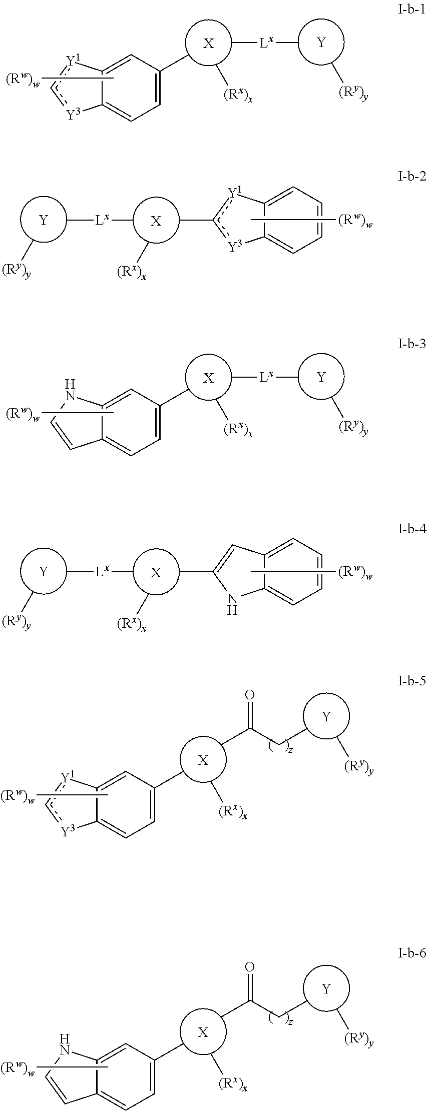 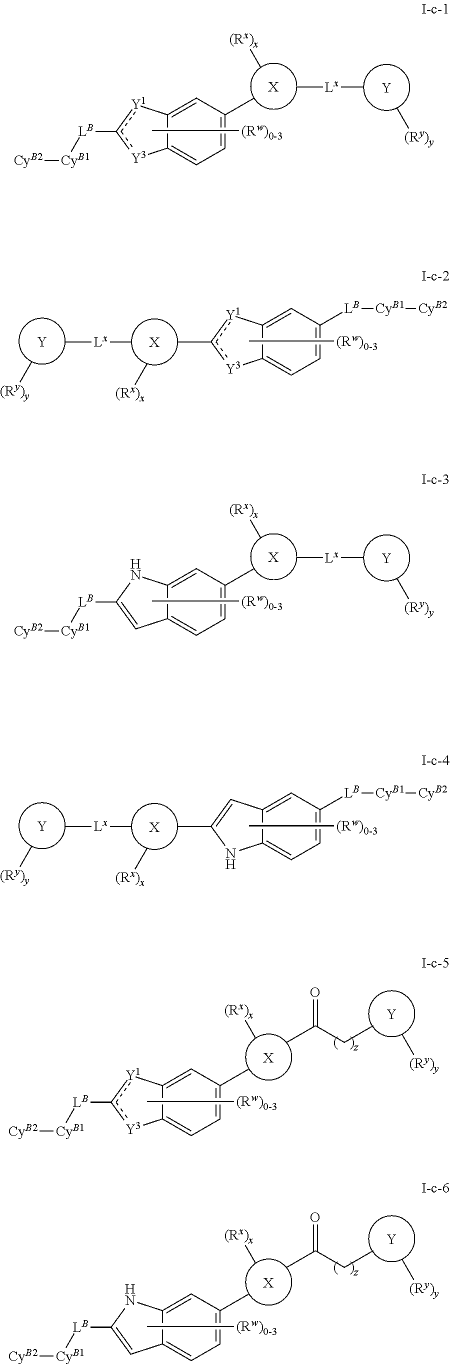 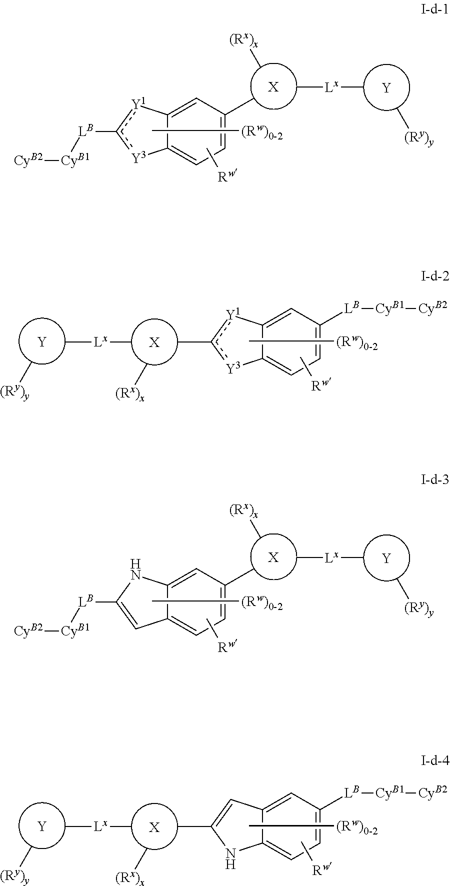 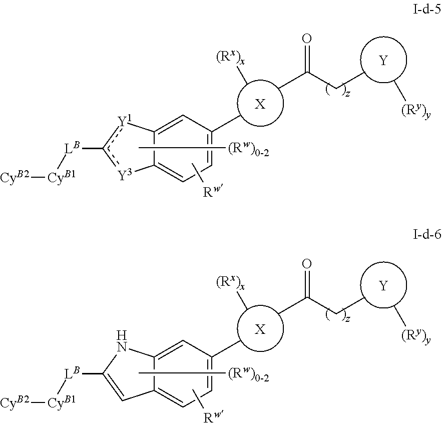 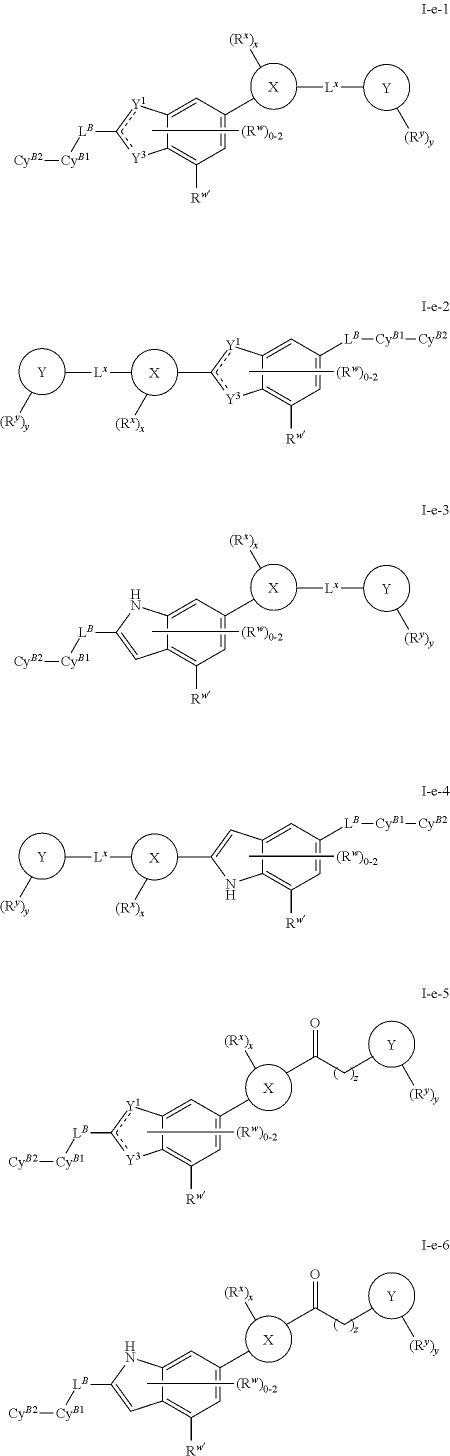
or a pharmaceutically acceptable salt thereof, wherein:
R^{w′} is hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

42-47. (canceled)

48. The method of claim 26, wherein:
(i) Ring W and its R^{w} substituents is 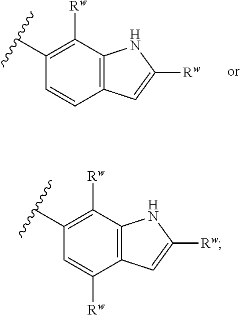
Ring Y is a 5-6 membered monocyclic heteroaryl with 1-4 heteroatoms independently selected from nitrogen, oxygen and sulfur;
z is 2;
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-7 membered saturated or partially unsaturated carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur; and
each Cy^{B2} is independently an optionally substituted ring selected from phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur; or
(ii) Ring W and its R^{w} substituents is 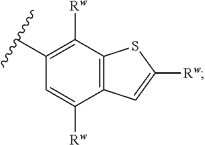
Ring Y is a 5-6 membered monocyclic heteroaryl with 1-4 heteroatoms independently selected from nitrogen, oxygen and sulfur;
z is 2;
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-7 membered saturated or partially unsaturated carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur; and
each Cy^{B2} is independently an optionally substituted ring selected from phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur; or
(iii) Ring W and its R^{w} substituents is 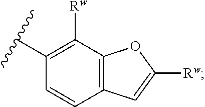
Ring Y is a 5-6 membered monocyclic heteroaryl with 1-4 heteroatoms independently selected from nitrogen, oxygen and sulfur;
z is 2;
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-7 membered saturated or partially unsaturated carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur; and
each Cy^{B2} is independently an optionally substituted ring selected from phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur.

49. The method of claim 25, wherein the inhibitor compound is selected from Table 1A or Table 1B, or a pharmaceutically acceptable salt thereof.

50. The method of claim 25, wherein the contacting occurs in a patient or a biological sample.
